(19) United States
(12) Patent Application Publication
(10) Pub. No.: US 2010/0291579 A1
(43) Pub. Date: Nov. 18, 2010

(54) Novel genes encoding proteins having prognostic, diagnostic, preventive, therapeutic, and other uses

(75) Inventors: Christopher C. Fraser, Lexington, MA (US); Sean Anthony McCarthy, San Diego, CA (US); John D. Sharp, Arlington, MA (US); Thomas M. Barnes, Brookline, MA (US); Nicholas C. Wrighton, Winchester, MA (US); Charles R. Mackay, Vaucluse (AU); Kevin R. Leiby, San Francisco, CA (US); Douglas A. Holtzman, Seattle, WA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 12/803,671

(22) Filed: Jul. 1, 2010

Related U.S. Application Data
(63) Continuation of application No. 12/319,689, filed on Jan. 9, 2009, now abandoned.
Application No. 12/319,689 is a continuation of application No. 11/265,762, filed on Nov. 2, 2005, now abandoned.
Application No. 11/265,762 is a continuation of application No. 09/796,753, filed on Mar. 1, 2001, now abandoned.
Application No. 09/796,753 is a continuation-in-part of application No. 09/183,175, filed on Oct. 30, 1998, now abandoned.
Application No. 09/183,175 is a continuation-in-part of application No. 09/599,596, filed on Jun. 22, 2000, now abandoned.
Application No. 09/599,596 is a division of application No. 09/223,546, filed on Dec. 30, 1998, now abandoned.
Application No. 09/223,546 is a continuation-in-part of application No. 09/471,179, filed on Dec. 23, 1999, now abandoned.
Application No. 09/471,179 is a continuation-in-part of application No. 09/223,546, filed on Dec. 30, 1998, now abandoned.
Application No. 09/796,753 is a continuation-in-part of application No. 09/474,072, filed on Dec. 29, 1999, now abandoned.
Application No. 09/474,072 is a continuation-in-part of application No. 09/224,246, filed on Dec. 30, 1998, now abandoned.
Application No. 09/796,753 is a continuation-in-part of application No. 09/474,071, filed on Dec. 29, 1999, now abandoned.
Application No. 09/474,071 is a continuation-in-part of application No. 09/223,094, filed on Dec. 30, 1998, now abandoned.
Application No. 09/796,753 is a continuation-in-part of application No. 09/514,010, filed on Feb. 25, 2000, now abandoned.
Application No. 09/514,010 is a continuation-in-part of application No. 09/259,388, filed on Feb. 26, 1999, now abandoned.
Application No. 09/796,753 is a continuation-in-part of application No. 09/516,745, filed on Mar. 1, 2000, now abandoned.
Application No. 09/796,753 is a continuation-in-part of application No. 09/597,993, filed on Jun. 19, 2000, now abandoned.
Application No. 09/597,993 is a continuation-in-part of application No. 09/336,536, filed on Jun. 18, 1999, now Pat. No. 6,406,884.
Application No. 09/796,753 is a continuation-in-part of application No. 09/630,334, filed on Jul. 31, 2000, now abandoned.
Application No. 09/630,334 is a continuation-in-part of application No. 09/365,164, filed on Jul. 30, 1999, now abandoned.
Application No. 09/796,753 is a continuation-in-part of application No. 09/665,666, filed on Sep. 20, 2000, now abandoned.
Application No. 09/665,666 is a continuation-in-part of application No. 09/399,723, filed on Sep. 20, 1999, now abandoned.
Application No. 09/796,753 is a continuation-in-part of application No. 09/677,751, filed on Oct. 2, 2000, now abandoned.
Application No. 09/677,751 is a continuation-in-part of application No. 09/409,634, filed on Sep. 30, 1999, now abandoned.
Application No. 09/796,753 is a continuation-in-part of application No. 09/572,002, filed on May 15, 2000, now abandoned.
Application No. 09/572,002 is a continuation-in-part of application No. 09/312,359, filed on May 14, 1999, now abandoned.
Application No. 09/796,753 is a continuation-in-part of application No. 09/606,565, filed on Jun. 29, 2000, now abandoned.
Application No. 09/606,565 is a continuation-in-part of application No. 09/342,687, filed on Jun. 29, 1999, now abandoned.
Application No. 09/796,753 is a continuation-in-part of application No. 09/606,317, filed on Jun. 29, 2000, now abandoned.
Application No. 09/606,317 is a continuation-in-part of application No. 09/345,464, filed on Jun. 30, 1999, now abandoned.
(60) Provisional application No. 60/122,458, filed on Mar. 1, 1999.

(51) Int. Cl. C12Q 1/68; G01N 33/53; C12P 21/06; C12N 5/10; C12N 5/02; C12N 15/63; C07K 14/435; C07K 16/18; C07K 1/00; C07H 21/04

(52) U.S. Cl. 435/6; 435/7.1; 435/69.1; 435/325; 435/375; 435/320.1; 530/350; 530/387.9; 530/402; 536/23.5

(57) ABSTRACT

The invention provides isolated nucleic acid molecules and polypeptide molecules. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides and antibodies. Diagnostic, screening and therapeutic methods utilizing compositions of the invention are also provided.

### CROSS REFERENCE TO RELATED APPLICATIONS

This application is a continuation-in-part of U.S. patent application Ser. No. 09/183,175, filed Oct. 30, 1998.

This application is a continuation-in-part of U.S. patent application Ser. No. 09/599,596, filed Jun. 22, 2000, which is a divisional of U.S. patent application Ser. No. 09/223,546, filed Dec. 30, 1998, and a continuation-in-part of U.S. patent application Ser. No. 09/471,179, filed Dec. 23, 1999, which is a continuation-in-part of U.S. patent application Ser. No. 09/223,546, filed Dec. 30, 1998.

This application is a continuation-in-part of U.S. patent application Ser. No. 09/474,072, filed Dec. 29, 1999, which is a continuation-in-part of U.S. patent application Ser. No. 09/224,246, filed Dec. 30, 1998.

This application is a continuation-in-part of U.S. patent application Ser. No. 09/474,071, filed Dec. 29, 1999, which is a continuation-in-part of U.S. patent application Ser. No. 09/223,094, filed Dec. 30, 1998.

This application is a continuation-in-part of U.S. patent application Ser. No. 09/514,010, filed Feb. 25, 2000, which is a continuation-in-part of U.S. patent application Ser. No. 09/259,388, filed Feb. 26, 1999.

This application is a continuation-in-part of U.S. patent application Ser. No. 09/516,745, filed Mar. 1, 2000, which claims the benefit of priority of U.S. Provisional Patent Application Ser. No. 60/122,458, filed Mar. 1, 1999.

This application is a continuation-in-part of U.S. patent application Ser. No. 09/597,993, filed Jun. 19, 2000, which is a continuation-in-part of U.S. patent application Ser. No. 09/336,536, filed Jun. 18, 1999.

This application is a continuation-in-part of U.S. patent application Ser. No. 09/630,334, filed Jul. 31, 2000, which is a continuation-in-part of U.S. patent application Ser. No. 09/365,164, filed Jul. 30, 1999.

This application is a continuation-in-part of U.S. patent Application Ser. No. 09/665,666, filed Sep. 20, 2000, which is a continuation-in-part of U.S. patent application Ser. No. 09/399,723, filed Sep. 20, 1999.

This application is a continuation-in-part of U.S. patent Application Ser. No. 09/677,751, filed Sep. 30, 2000, which is a continuation-in-part of U.S. patent application Ser. No. 09/409,634, filed Sep. 30, 1999.

This application is a continuation-in-part of U.S. patent application Ser. No. 09/572,002, filed May 14, 2000, which is a continuation-in-part of U.S. patent application Ser. No. 09/312,359, filed May 14, 1999.

This application is a continuation-in-part of U.S. patent application Ser. No. 09/606,565, filed Jun. 29, 2000, which is a continuation-in-part of U.S. patent application Ser. No. 09/342,687, filed Jun. 29, 1999.

This application is a continuation-in-part of U.S. patent application Ser. No. 09/606,317, filed Jun. 29, 2000, which is a continuation-in-part of U.S. patent application Ser. No. 09/345,464, filed Jun. 30, 1999.

Each of the applications cross-referenced in this section are incorporated into this disclosure by reference in its entirety.

### BACKGROUND OF THE INVENTION

Many secreted proteins, for example, cytokines and cytokine receptors, play a vital role in the regulation of cell growth, cell differentiation, and a variety of specific cellular responses. A number of medically useful proteins, including erythropoietin, granulocyte-macrophage colony stimulating factor, human growth hormone, and various interleukins, are secreted proteins. Thus, an important goal in the design and development of new therapies is the identification and characterization of secreted and transmembrane proteins and the genes which encode them

Many secreted proteins are receptors which bind a ligand and transduce an intracellular signal, leading to a variety of cellular responses. The identification and characterization of such a receptor enables one to identify both the ligands which bind to the receptor and the intracellular molecules and signal transduction pathways associated with the receptor, permitting one to identify or design modulators of receptor activity, e.g., receptor agonists or antagonists and modulators of signal transduction.

### SUMMARY OF THE INVENTION

The present invention is based, at least in part, on the discovery of cDNA molecules which encode the INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 proteins, all of which are either wholly secreted or transmembrane polypeptides.

The TANGO 214 proteins share significant homology to the human HtrA protein, the human homologue of the E. coli HtrA (high temperature requirement) gene product, a critical component of the bacterial response to stress. Because of their homology to the human HtrA protein, TANGO 214 proteins (and the nucleic acids that encode them) are referred to herein as HtrA-2 proteins (and nucleic acid molecules).

The TANGO 253 proteins are Clq domain-containing polypeptides that exhibit homology to a human adipocyte complement-related protein precursor.

The TANGO 257 proteins are homologous to the human extracellular molecule olfactomedin, a molecule important in the maintenance, growth and differentiation of chemosensory cilia of olfactory neurons.

The INTERCEPT 258 proteins are Ig domain-containing polypeptides that exhibit homology to an antigen (A33) expressed in colonic and small bowel epithelium, a protein that may represent a cancer cell marker.

The TANGO 339 proteins are transmembrane 4 domain-containing polypeptides that exhibit homology to human CD9 antigen, a cell surface antigen associated with platelet activation and aggregation.

The TANGO 358, and TANGO 365 proteins are transmembrane proteins.

The TANGO 368 proteins are secreted proteins encoded by sequences with homology to genomic sequences of the human T-cell receptor gamma V1 gene region.

The TANGO 383 proteins are transmembrane polypeptides with homology to retinopathy proteins.

The MANGO 346, MANGO 349, and TANGO 369 proteins are secreted proteins.

The INTERCEPT 307 are transmembrane proteins that are related to the prostate cancer upregulated PB39 gene product.

The MANGO 511 proteins are related to the leukocyte Ig-like receptors (LIRs) which bind MHC class I.

The TANGO 361 proteins are Trypsin domain-containing polypeptides that exhibit homology to human serine proteases which belong to the trypsin-like protease family.

The TANGO 499 proteins are GDNF-like domain-containing polypeptides that exhibit homology to human Persephin, Artemin, Neurturin and GDNF, cell surface antigens associated with embryogenesis and development.

The TANGO 315 proteins are transmembrane polypeptides related to CD33 polypeptides and the Ob binding protein.

The TANGO 330 proteins are transmembrane and secreted polypeptides and are related to roundabout polypeptides.

The TANGO 437 proteins are transmembrane polypeptides containing ion transport, cell cycle protein and putative permease domains.

The TANGO 480 proteins are transmembrane polypeptides containing NADH-Ubiquinone/plastoquinone (complex 1) domains.

The INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 proteins, fragments, derivatives, and variants thereof of the present invention are collectively referred to herein as “polypeptides of the invention” or “proteins of the invention.”

Nucleic acid molecules encoding the polypeptides or proteins of the invention are collectively referred to as “nucleic acids of the invention.” The nucleic acids and polypeptides of the present invention are useful as modulating agents in regulating a variety of cellular processes. Accordingly, in one aspect, this invention provides isolated nucleic acid molecules encoding a polypeptide of the invention or a biologically active portion thereof. The present invention also provides nucleic acid molecules which are suitable for use as primers or hybridization probes for the detection of nucleic acids encoding a polypeptide of the invention.

The present invention is based, at least in part, on the discovery of human cDNA molecules which encode proteins which are herein designated INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499. These proteins, fragments thereof, derivatives thereof, and variants thereof are collectively referred to herein as the polypeptides of the invention or the proteins of the invention. Nucleic acid molecules encoding polypeptides of the invention are collectively referred to as nucleic acids of the invention.

The nucleic acids and polypeptides of the present invention are useful as modulating agents for regulating a variety of cellular processes. Accordingly, in one aspect, the present invention provides isolated nucleic acid molecules encoding a polypeptide of the invention or a biologically active portion thereof. The present invention also provides nucleic acid molecules which are suitable as primers or hybridization probes for the detection of nucleic acids encoding a polypeptide of the invention.

The invention includes fragments of any of the nucleic acids described herein wherein the fragment retains a biological or structural function by which the full-length nucleic acid is characterized (e.g., an activity, an encoded protein, or a binding capacity). The invention furthermore includes fragments of any of the nucleic acids described herein wherein the fragment has a nucleotide sequence sufficiently (e.g., 50%, 60%, 70%, 80%, 85%, 90%, 95%, 98%, or 99% or greater) identical to the nucleotide sequence of the corresponding full-length nucleic acid that it retains a biological or structural function by which the full-length nucleic acid is characterized (e.g., an activity, an encoded protein, or a binding capacity).

The invention includes fragments of any of the polypeptides described herein wherein the fragment retains a biological or structural function by which the full-length polypeptide is characterized (e.g., an activity or a binding capacity). The invention furthermore includes fragments of any of the polypeptides described herein wherein the fragment has an amino acid sequence sufficiently (e.g., 50%, 60%, 70%, 80%, 85%, 90%, 95%, 98%, or 99% or greater) identical to the amino acid sequence of the corresponding full-length polypeptide that it retains a biological or structural function by which the full-length polypeptide is characterized (e.g., an activity or a binding capacity).

The invention also features nucleic acid molecules which are at least 40% (or 50%, 60%, 70%, 80%, 90%, 95%, or 98%) identical to the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, the TANGO 136 nucleotide sequence of the cDNA insert of a clone deposited on Sep. 11, 1998 with the ATCC® as accession no. 98880, the TANGO 128, TANGO 140, TANGO 197 and TANGO 214 nucleotide sequences of cDNA inserts of clones deposited on Nov. 20, 1998 with the ATCC® as accession no. 98999, the TANGO 212 nucleotide sequence of the cDNA insert of a clone deposited on Sep. 10, 1998 with the ATCC® as accession no. 202171, the TANGO 213 nucleotide sequence of the cDNA insert of a clone deposited on Oct. 30, 1998 with the ATCC®& as accession no. 98965, the TANGO 224 nucleotide sequence of the cDNA insert of a clone deposited on Oct. 30, 1998 with the ATCC® as accession no. 98966, the TANGO 176 nucleotide sequence of the cDNA insert of a clone deposited on Jan. 7, 1999 with the ATCC® as accession no. 207042, the TANGO 221 nucleotide sequence of the cDNA insert of a clone deposited on Jan. 7, 1999 with the ATCC® as accession no. 207044, the TANGO 222 nucleotide sequence of the cDNA insert of a clone deposited on Jan. 7, 1999 with the ATCC® as accession no. 207043, the TANGO 201 and TANGO 223 nucleotide sequence of the cDNA insert of a clone deposited on Jan. 22, 1999 with the ATCC® as accession no. 207081, the TANGO 216, TANGO 261, TANGO 262, TANGO 266 and TANGO 267 nucleotide sequence of the cDNA insert of a clone deposited on Mar. 26, 1999 with the ATCC® as accession no. 207176, the TANGO 253, TANGO 257, and INTERCEPT 258 nucleotide sequences of cDNA inserts of clones deposited on Apr. 21, 1999 with the ATCC® as accession no. 207222, the TANGO 253 nucleotide sequence of the cDNA insert of a clone deposited on Apr. 21, 1999 with the ATCC® as accession no. 207215, the TANGO 257 nucleotide sequence of the cDNA insert of a clone deposited on Apr. 21, 1999 with the ATCC® as accession no. 207217, the INTERCEPT 258, TANGO 206 and TANGO 209 nucleotide sequences of cDNA inserts of clones deposited on Apr. 21, 1999 with the ATCC® as accession no. 207221, the TANGO 204 nucleotide sequence of the cDNA insert of a clone deposited on Apr. 21, 1999 with the ATCC® as accession no. 207192, the TANGO 204 nucleotide sequence of the cDNA insert of a clone deposited on Apr. 21, 1999 with the ATCC® as accession no. 207189, the TANGO 206, TANGO 209, MANGO 245, TANGO 244 and TANGO 246 nucleotide sequence of the cDNA insert of a clone deposited on Apr. 21, 1999 with the ATCC® as accession no. 207223, the TANGO 275 nucleotide sequence of the cDNA insert of a clone deposited on Apr. 21, 1999 with the ATCC® as accession no. 207220, the INTERCEPT 340, MANGO 347 and TANGO 272 nucleotide sequences of cDNA inserts of clones deposited on Jun. 18, 1999 with the ATCC® as accession no. PTA-250, the MANGO 003 nucleotide sequence of the cDNA insert of a clone deposited on Mar. 27, 1999 with the ATCC® as accession no. 207178, the TANGO 295 nucleotide sequence of the cDNA insert of a clone deposited on Jun. 18, 1999 with the ATCC® as accession no. PTA-249, the TANGO 339 and TANGO 358 nucleotide sequences of cDNA inserts of clones deposited on Jun. 29, 1999 with the ATCC® as accession no. PTA-292, the MANGO 346, TANGO 365 and TANGO 368 nucleotide sequence of the cDNA insert of a clone deposited on Jun. 29, 1999 with the ATCC® as accession no. PTA-291, the MANGO 349, TANGO 369 and TANGO 383 nucleotide sequence of the cDNA insert of a clone deposited on Jun. 29, 1999 with the ATCC® as accession no. PTA-295, the INTERCEPT 307 and TANGO 499, form 1, variant 1 nucleotide sequences of cDNA inserts of clones deposited on Jun. 29, 1999 with the ATCC® as accession no. PTA-455, the TANGO 361 nucleotide sequence of the cDNA insert of a clone deposited on Jun. 29, 1999 with the ATCC® as accession no. PTA-438, the TANGO 499, from 2, variant 3 nucleotide sequence of the cDNA insert of a clone deposited on Aug. 5, 1999 with the ATCC® as accession no. PTA-454, the MANGO 511 nucleotide sequence of the cDNA insert of a clone deposited on Jul. 23, 1999 with the ATCC® as accession no. PTA-425, the TANGO 315, TANGO 437, TANGO 330 and TANGO 480 nucleotide sequences of cDNA inserts of clones deposited on Oct. 1, 1999 with the ATCC® as accession no. PTA-816, or a complement thereof.

These deposited nucleotide sequences are hereafter individually and collectively referred to as “the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816.”

The invention features nucleic acid molecules which include a fragment of at least (25, 40, 60, 80, 100, 150, 200, 250, 300, 350, 400, 450, 500, 600, 700, 800, 900, 1000, 1200, 1400, 1600, 1800, 2000, 2200, 2400, 2600, 2800, 3000, 3500, 4000, 4500, 5000, or more) consecutive nucleotide residues of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-30291, PTA-295, PTA-455, PTA-438, PTA-454, PTA-425, and PTA-816, or a complement thereof.

The invention also features nucleic acid molecules which include a nucleotide sequence encoding a protein having an amino acid sequence that is at least 50% (or 60%, 70%, 80%, 90%, 95%, or 98%) identical to the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the nucleotide sequence of any of the clones deposited as ATCC® Accession numbers 98880, 98999, 202171, 98965, 98966, 98899, 207042, 207044, 5207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816 or a complement thereof.

In certain embodiments, the nucleic acid molecules have the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 15207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816.

Also within the invention are nucleic acid molecules which encode a fragment of a polypeptide having the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, the fragment including at least 10 (12, 15, 20, 25, 30, 40, 50, 75, 100, 125, 150, 200, 250, 300, 400, 500, 750, 1000 or more) consecutive amino acid residues of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164.

The invention includes nucleic acid molecules which encode a naturally occurring allelic variant of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, wherein the nucleic acid molecule hybridizes under stringent conditions to a nucleic acid molecule having a nucleic acid sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof.

Also within the invention are isolated polypeptides or proteins having an amino acid sequence that is at least about 50%, preferably 60%, 75%, 90%, 95%, or 98% identical to the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164.

Also within the invention are isolated polypeptides or proteins which are encoded by a nucleic acid molecule having a nucleotide sequence that is at least about 40%, preferably 50%, 60%, 75%, 85%, or 95% identical the nucleic acid sequence encoding any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, and isolated polypeptides or proteins which are encoded by a nucleic acid molecule consisting of the nucleotide sequence which hybridizes under stringent hybridization conditions to a nucleic acid molecule having the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816.

Also within the invention are polypeptides which are naturally occurring allelic variants of a polypeptide that includes the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 5104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, wherein the polypeptide is encoded by a nucleic acid molecule which hybridizes under stringent conditions to a nucleic acid molecule having the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof.

The invention also features nucleic acid molecules that hybridize under stringent conditions to a nucleic acid molecule having the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, and the nucleotide sequence of any of the clones deposited as ATCC® Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof. In some embodiments, the isolated nucleic acid molecules encode a cytoplasmic, transmembrane, extracellular, or other domain of a polypeptide of the invention. In other embodiments, the invention provides an isolated nucleic acid molecule which is antisense to the coding strand of a nucleic acid of the invention.

The invention features nucleic acid molecules of at least 570, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800 or 2835 nucleotides of the nucleotide sequence of the cDNA, the nucleotide sequence of the TANGO 128 cDNA clone of ATCC® Accession No. 98999, or a complement thereof. The invention also features nucleic acid molecules comprising at least 25, 50, 100, 150, 200, 250, 300, 350, 400, 450, 500, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200 or 2230 nucleotides of nucleic acids 1 to 2233 of SEQ ID NO:5, or a complement thereof.

The invention features nucleic acid molecules which include a fragment of at least 15, 25, 50, 75, 100, 150, 200, 250, 300, 350, 400, 450, 500, 550, 600, 650, 700, 750, 800, 850, 900, 950, 1000 or 1030 nucleotides of the nucleotide sequence of the human TANGO 128 open reading frame (ORF) of SEQ ID NO:5, or a complement thereof.

The invention features nucleic acid molecules of at least 250, 275, 300, 325, 350, 375, 400, 425, 450, 475, 500, 525, 550, 575, 600, 625, 650, 675, 700, 725, 750 or 760 nucleotides of the nucleotide sequence of SEQ ID NO:21, the nucleotide sequence of a mouse TANGO 128 cDNA, or a complement thereof. The invention features nucleic acid molecules comprising at least 25 30, 35, 40, 45, 50, 55, 60, 65, 70 or 77 nucleotides of nucleic acids 1 to 78 of mouse TANGO 128 cDNA, or a complement thereof. The invention features nucleic acid molecules comprising at least 25 30, 35, 40, 45, 50, 55 or 60 nucleotides of nucleic acids 257 to 318 of SEQ ID NO:21, or a complement thereof.

The invention features nucleic acid molecules comprising at least 225, 250, 275, 300, 325, 350, 375, 400, 425, 450, 475, 500, 525 or 550 nucleotides of the nucleotide sequence of the open reading frame of SEQ ID NO:21, or a complement thereof. The invention also features nucleic acid molecules comprising at least 25, 30, 35, 40, 45, 50, 55 or 60 nucleotides of nucleic acids 46 to 107 of the open reading frame of SEQ ID NO:21, or a complement thereof.

The invention features nucleic acid molecules of at least 425, 450, 475, 500, 525, 550, 600, 650, 700, 750, 800, 850, 900, 950, 1000, 1100, 1200, 1300, 1400, 1500 or 1540 nucleotides of the nucleotide sequence of SEQ ID NO:7, the nucleotide sequence of SEQ ID NO:7, the nucleotide sequence of the TANGO 140-1 cDNA clone of ATCC®& Accession No. 98999, or a complement thereof. The invention also features nucleic acid molecules comprising at least 25, 50, 100, 150, 200, 250, 300, 350 400, 450, 500 or 540 nucleotides of nucleic acids 1 to 545 of SEQ ID NO:7, or a complement thereof. The invention also features nucleic acid molecules comprising at least 25, 50, 100, 150, 200, 250, 300, 350, 400, 450, 500, 550 or 580 nucleotides of nucleic acids 980 to 1550 of SEQ ID NO:7, or a complement thereof.

The invention features nucleic acid molecules of at least 425, 450, 475, 500, 525, 550, 600, 650, 700, 750, 800, 850, 900, 950, 1000, 1100, 1200, 1300, 1400, 1500, 1650, 1700, 1750, 1800, 1850, 1900, 1950, 2000, 2050, 2100, 2150, 2200, 2300, 2350, 2400, 2450, 2500, 2550, 2600, 2650, 2700, 2750, 2800, 2850, 2900, 2950, 3000, 3050, 3100, 3150, 3200, 3250, 3300, 3350 or 3385 nucleotides of the nucleotide sequence of SEQ ID NO:9, the nucleotide sequence of SEQ ID NO:9, the nucleotide sequence of the TANGO 140-2 cDNA clone of ATCC® Accession No. 98999, or a complement thereof. The invention also features nucleic acid molecules comprising at least 25, 50, 100, 150, 200, 250, 300, 350 400, 450, 500 or 540 nucleotides of nucleic acids 1 to 545 of SEQ ID NO:9, or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1100, 1200, 1300, 1400, 1500, 1650, 1700, 1750, 1800, 1850, 1900, 1950, 2000, 2050, 2100, 2150, 2200, 2300, 2350 or 2400 nucleotides of nucleic acids 980 to 3385 of SEQ ID NO:9, or a complement thereof.

The invention features nucleic acid molecules comprising at least 75, 100, 150, 200, 250, 300, 350, 400, 450, 500, 550, 600 or 615 nucleotides of the nucleotide sequence of SEQ ID NOs:7 or 9, or a complement thereof. The invention features nucleic acid molecules comprising at least 25, 75, 100, 150, 200, 250, 300, 350, 400, 450, 500 or 545 nucleotides of nucleic acids 1 to 545 of human TANGO 140-1 or 140-2 ORFs of SEQ ID NOs:7 or 9, or a complement thereof.

The invention features nucleic acid molecules of at least 520, 550, 600, 650, 700, 750, 800, 850, 900, 950, 1000, 1100, 1200, 1300, 1400, 1500, 1600, 1700, 1800, 1900, 2000, 2100, 2200, 2250 or 2270 nucleotides of the nucleotide sequence of SEQ ID NO: 11, the nucleotide sequence of SEQ ID NO:11, the TANGO 197 cDNA clone of ATCC® Accession No. 98999, or a complement thereof. The invention also features nucleic acid molecules comprising at least 25, 50, 100, 150, 200, 250, 300, 350, 400, 450, 500, 550, 600, 650, 700, 750 or 785 nucleotides of nucleic acids 1 to 789 of SEQ ID NO:11, or a complement thereof. The invention also features nucleic acid molecules comprising at least 25, 50, 100, 150, 200, 250, 300, 350, 400, 450 or 500 nucleotides of nucleic acids 1164 to 1669 of SEQ ID NO:11, or a complement thereof. The invention also features nucleic acid molecules comprising at least 25, 50 or 80 nucleotides of nucleic acids 2190 to 2272 of SEQ ID NO:11, or a complement thereof.

The invention features nucleic acid molecules which include a fragment of at least 380, 400, 450, 500, 550, 600, 650, 700, 750, 800, 850, 900, 950, 1000, 1100, 1200, 1300, 1400, 1500, 1600, 1700, 1750 or 1770 nucleotides of the nucleotide sequence of the TANGO 197 ORF of SEQ ID NO: 1, or a complement thereof. The invention also features nucleic acid molecules comprising at least 25, 50, 100, 150, 200, 250, 300, 350, 400, 450, 500, 550 or 575 nucleotides of nucleic acids 1 to 576 of the TANGO 197 ORF of SEQ ID NO: 11, or a complement thereof.

The invention features nucleic acid molecules of at least 515, 550, 600, 650, 700, 750, 800, 850, 900, 950, 1000, 1100, 1200, 1300, 1400, 1500, 1600, 1700, 1800, 1900, 2000, 2100, 2200, 2250, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3500, 3550, 3600, 3650, 3700, 3750, 3800, 3850, 3900, 3950, 4000, 4050, 4100, 4150, 4200, 4250, 4300, 4350, 4400 or 4415 nucleotides of the nucleotide sequence of SEQ ID NO:23, the nucleotide sequence of SEQ ID NO:23, the nucleotide sequence of a mouse TANGO 197 cDNA, or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, 2550, 2600, 2650, 2700, 2750, 2800, 2850, 2900, 2950, 3000, 3050, 3100 or 3135 nucleotides of nucleic acids 1 to 3138 of SEQ ID NO:23, or a complement thereof. The invention also features nucleic acid molecules comprising at least 25, 50, 100, 150, 200, 250, 300 or 320 nucleotides of nucleic acids 4094 to 4417 of SEQ ID NO:23, or a complement thereof.

The invention features nucleic acid molecules which include a fragment of at least 390, 400, 450, 500, 550, 600, 650, 700, 750, 800, 850, 900, 950, 1000, 1100 or 1140 nucleotides of the nucleotide sequence of the mouse TANGO 197 ORF of SEQ ID NO:23, or a complement thereof.

The invention features nucleic acid molecules of at least 545, 600, 650, 700, 750, 800, 850, 900, 950, 1000, 1100, 1200, 1300, 1400, 1500, 1600, 1700, 1800, 1900, 2000, 2100, 2200, 2250, 2250, 2300, 2350, 2400 or 2435 nucleotides of the nucleotide sequence of SEQ ID NO: 13, the nucleotide sequence of SEQ ID NO: 13, the nucleotide sequence of the TANGO 212 cDNA clone of ATCC® Accession No. 202171 or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1050, 1100, 1150, 1200, 1250 or 1270 nucleotides of nucleic acids 1 to 1273 of SEQ ID NO:13, or a complement thereof. The invention also features nucleic acid molecules comprising at least 25, 50, 100, 150, 200, 250, 300 or 320 nucleotides of nucleic acids 4094 to 4417 of SEQ ID NO:13, or a complement thereof.

The invention features nucleic acid molecules which include a fragment of at least 240, 275, 300, 350, 400, 450, 500, 550, 600, 650, 700, 750, 800, 850, 900, 950, 1000, 1100, 1150, 1200, 1250, 1300, 1350, 1400, 1450, 1500, 1550, 1600 or 1660 nucleotides of the nucleotide sequence of the TANGO 212 ORF of SEQ ID NO:13, or a complement thereof.

The invention also features nucleic acid molecules comprising at least 25, 50, 100, 150, 200, 250, 300, 350, 400, 450, 500, 550, 600, 650, 700, 750, 800, 850 or 900 nucleotides of nucleic acids 1 to 905 of the TANGO 212 ORF of SEQ ID NO:13, or a complement thereof.

The invention features nucleic acid molecules of at least 785, 800, 850, 900, 950, 1000, 1050, 1100, 1150 or 1180 nucleotides of the nucleotide sequence of SEQ ID NO:25, the nucleotide sequence of SEQ ID NO:25, the nucleotide sequence of a mouse TANGO 212 cDNA, or a complement thereof. The invention also features nucleic acid molecules comprising at least 25, 50, 100, 150 or 190 nucleotides of nucleic acids 983 to 1180 of SEQ ID NO:25, or a complement thereof.

The invention features nucleic acid molecules which include a fragment of at least 570, 600, 650, 700, 750, 800, 850, 900, 950 or 998 nucleotides of the nucleotide sequence of the TANGO 212 ORF of SEQ ID NO:25, or a complement thereof. The invention also features nucleic acid molecules comprising at least 25, 50, 100, 150 or 180 nucleotides of nucleic acids 804 to 999 of the TANGO 212 ORF of SEQ ID NO:25, or a complement thereof.

The invention features nucleic acid molecules of at least 530, 600, 650, 700, 750, 800, 850, 900, 950, 1000, 1100, 1200, 1300, 1400 or 1495 nucleotides of the nucleotide sequence of SEQ ID NO: 15, the nucleotide sequence of the TANGO 213 cDNA clone of ATCC® Accession No. 98965, or a complement thereof. The invention also features nucleic acid molecules comprising at least 25, 50, 100, 150, 200, 250, 300 or 360 nucleotides of nucleic acids 1 to 361 of SEQ ID NO: 15, or a complement thereof. The invention also features nucleic acid molecules comprising at least 25, 40, 50 or 60 nucleotides of nucleic acids 759 to 822 of SEQ ID NO: 15, or a complement thereof.

The invention features nucleic acid molecules which include a fragment of at least 250, 275, 300, 350, 400, 450, 500, 550, 600, 650, 700, 750, 800 or 810 nucleotides of the nucleotide sequence of the TANGO 213 ORF of SEQ ID NO: 15, or a complement thereof.

The invention also features nucleic acid molecules comprising at least 25, 50, 100, 150, 200, 250 or 300 nucleotides of nucleic acids 1 to 304 of the TANGO 213 ORF of SEQ ID NO: 15, or a complement thereof. The invention also features nucleic acid molecules comprising at least 25, 40, 50 or 60 nucleotides of nucleic acids 701 to 764 of the TANGO 213 ORF of SEQ ID NO: 15, or a complement thereof.

The invention features nucleic acid molecules of at least 530, 600, 650, 700, 750, 800, 850, 900, 950, 1000, 1100, 1200, 1300, 1400, 1500, 1600, 1700, 1800, 1900, 2000, 2100 or 2150 nucleotides of the nucleotide sequence of SEQ ID NO:27, the nucleotide sequence of a mouse TANGO 213 cDNA, or a complement thereof. The invention also features nucleic acid molecules comprising at least 25, 50, 100, 150, 200, 250, 300, 350, 400, 450, 500, 550, 600, 650, 700, 750, 800, 850, 900, 950 or 1000 nucleotides of nucleic acids 1 to 1018 of SEQ ID NO:27, or a complement-thereof. The invention also features nucleic acid molecules comprising at least 25, 50, 100, 150, 200, 250, 300, 350, 400, 450, 500, 550, 600, 650, 700, 750, 800, 850, 900 or 920 nucleotides of nucleic acids 1227 to 2154 of SEQ ID NO:27, or a complement thereof.

The invention features nucleic acid molecules which include a fragment of at least 25, 50, 100, 150, 200, 250, 275, 300, 350, 400, 450, 500, 550 or 575 nucleotides of the nucleotide sequence of mouse TANGO 213 ORF of SEQ ID NO:27, or a complement thereof.

The invention features nucleic acid molecules of at least 570, 600, 650, 700, 750, 800, 850, 900, 950, 1000, 1100, 1200, 1300, 1400, 1500, 1600, 1700, 1800, 1900, 2000, 2100, 2150, 2200, 2250, 2300, 2350, 2400, 2450, 2500, 2550, 2600, 2650 or 2680 nucleotides of the nucleotide sequence of SEQ ID NO: 17, the nucleotide sequence of a human TANGO 224 cDNA form 1 or form 2 respectively, the nucleotide sequence of the TANGO 213 cDNA clone of ATCC® Accession Number 98966, or a complement thereof.

The invention also features nucleic acid molecules comprising at least 25, 50, 100, 150, 200, 250 or 270 nucleotides of nucleic acids 1 to 272 of SEQ ID NO:17, or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1050, 1100, 1150, 1200, 1250, 41300, 1350, 1400, 1450, 1500 or 1530 nucleotides of nucleic acids 573 to 2106 of SEQ ID NO:17, or a complement thereof.

The invention features nucleic acid molecules which include a fragment of at least 300, 350, 400, 450, 500, 550, 600, 650, 700, 750, 800, 850, 900, 950, 1000, 1050, 1100, 1150, 1200, 1250, 1300 or 1360 nucleotides of the nucleotide sequence of human TANGO 224 form 1 ORF of SEQ ID NO: 17, or a complement thereof. The invention also features nucleic acid molecules comprising at least 25, 40, 50, 100, 150 or 200 nucleotides of nucleic acids 1 to 204 of human TANGO 224 form 1 ORF of SEQ ID NO: 17, or a complement thereof. The invention also features nucleic acid molecules comprising at least 25, 40, 50, 100, 150, 200, 250, 300, 350, 400, 450, 500, 550, 600, 650, 700, 750, 800, 850, 900 or 930 nucleotides of nucleic acids 507 to 1440 of human TANGO 224 form 1 ORF of SEQ ID NO:17, or a complement thereof.

The invention features nucleic acid molecules which include a fragment of at least 570, 600, 650, 700, 750, 800, 850, 900, 950, 1000, 1100, 1200, 1300, 1400, 1500, 1600, 1700, 1800, 1900, 2000, 2100, 2150, 2200, 2250, 2300, 2350, 2400, 2450, 2500, 2550, 2600, 2650 or 2680 nucleotides of the nucleotide sequence of human TANGO 224 form 2 ORF of SEQ D NO: 19, or a complement thereof. The invention also features nucleic acid molecules comprising at least 25, 40, 50, 100, 150 or 200 nucleotides of nucleic acids 1 to 204 of human TANGO 224 form 2 ORF of SEQ ID NO:19, or a complement thereof. The invention also features nucleic acid molecules comprising at least 25, 40, 50, 100, 150, 200, 5250, 300, 350, 400, 450, 500, 550, 600, 650, 700, 750, 800, 850, 900, 950, 1000, 1050, 1100, 1150, 1200, 1250, 1300, 1350, 1400, 1450, 1500 or 1530 nucleotides of nucleic acids 507 to 2038 of human TANGO 224 form 2 ORF of SEQ ID NO: 19, or a complement thereof.

The invention features nucleic acid molecules of at least 510, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, or 2570 nucleotides of the nucleotide sequence of SEQ ID NO:31, the nucleotide sequence of a human HtrA-2 cDNA, the nucleotide sequence of the human HtrA-2 cDNA clone of ATCC® Accession No. 98899, or a complement thereof. The invention also features nucleic acid molecules comprising at least 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, or 910 nucleotides of nucleic acids 1 to 925 of SEQ ID NO:31, or a complement thereof.

The invention features nucleic acid molecules of at least 380, 400, 450, 500, 550, 600, 650, 700, 750, 800, 850, 900, 950, 1000, 1050, 1100, 1150, 1200, 1250, 1300, 1350, 1400, 1450, 1500, 1550, 1575, or 1595 nucleotides of the nucleotide sequence of SEQ ID NO:33, the nucleotide sequence of a mouse HtrA-2 cDNA, or a complement thereof. The invention also features nucleic acid molecules comprising at least 25, 50, 75, 100, 125, 150, 175, 200, 225, 250, 275, or 280 nucleotides of nucleic acids 1 to 285 of SEQ ID NO:33, or a complement thereof.

The invention features nucleic acid molecules of at least 525, 550, 600, 650, 700, 750, 800, 850, 900, 950, 1000, 1025, 1050, or 1070 nucleotides of the nucleotide sequence of SEQ ID NO:35, the nucleotide sequence of the human TANGO 221 cDNA clone of ATCC® Accession No. 207044, or a complement thereof. The invention also features nucleic acid molecules comprising at least 25, 50, 75, 100, 125, 150, 175, 200, 225, 250, 275, 300, 325, 350, 375, 400, 425, 450, 475, 500, or 510 nucleotides of nucleic acids 1 to 515 of SEQ ID NO:35, or a complement thereof.

The invention features nucleic acid molecules of at least 210, 225, 250, 275, 300, 325, 350, 375, 400, 425, 450, 475, 500, 525, 550, 575, 600, 625, 650, 675, 700, 725, 750, or 761 nucleotides of the nucleotide sequence of SEQ ID NO:37, the nucleotide sequence of a human TANGO 222 cDNA, the nucleotide sequence of the TANGO 222 cDNA clone of ATCC® Accession No. 207043, or a complement thereof. The invention also features nucleic acid molecules comprising at least 15, 20, 25, 30, or 35 nucleotides of nucleic acids 1 to 40 of SEQ ID NO:37, or a complement thereof.

The invention features nucleic acid molecules of at least 680, 700, 750, 800, 850, 900, 950, 1000, 1050, 1100, 1150, 1200, 1250, 1300, 1350, 1400, 1450, 1500, 1550, 1600, 1650, 1675, or 1695 nucleotides of the nucleotide sequence of SEQ ID NO:39, the nucleotide sequence of the human TANGO 176 cDNA clone of ATCC® Accession No. 207042, or a complement thereof. The invention also features nucleic acid molecules comprising at least 25, 50, 75, 100, 125, 150, 175, 200, 225, 250, 275, 300, 325, 350, 375, 400, 425, 450, 475, 500, 525, 550, 575, 600, 625, or 640 nucleotides of nucleic acids 1 to 645 of SEQ ID NO:39, or a complement thereof.

The invention features nucleic acid molecules which include a fragment of at least 810, 850, 900, 950, 1000, 1050, 1100, 1150, 1200, 1250, 1300, 1350, 1400, 1450, 1460, or 1470 nucleotides of the nucleotide sequence of a mouse TANGO 176 ORF, or a complement thereof.

The invention features nucleic acid molecules of at least 625, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, or 3677 nucleotides of the nucleotide sequence of SEQ ID NO:51, the nucleotide sequence of the human TANGO 216 cDNA clone of ATCC® Accession No. 207176, or a complement thereof. The invention also features nucleic acid molecules comprising at least 25, 50, 100, 150, 200, 250, 300, 350, 400, 450, 500, 600, 650, 700, 750, 800, 850, 900, 950, 1000, or 1040 nucleotides of nucleic acids 1695 to 2737 of SEQ ID NO:51, or a complement thereof, wherein such nucleic acid molecules encode polypeptides or proteins that exhibit at least one structural and/or functional feature of a polypeptide of the invention.

The invention features nucleic acid molecules of at least 675, 700, 725, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500 or 3501 nucleotides of the nucleotide sequence of SEQ ID NO:53, the nucleotide sequence of a mouse TANGO 216 cDNA, or a complement thereof. The invention features nucleic acid molecules comprising at least 85, 100, 125, 150, 175, 200, 225, 250, 275, 300, 325, 350, 375, 400, 425, 450, 475, 500, 525, 550, 575, 600, 625, 650, 675, 700, 725, 775, 800, 825, 850, 875, 900, 925, 950, 975, 1000, 1025, 1050, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400 nucleotides of nucleic acids 1 to 2417 of SEQ ID NO:53, or a complement thereof.

The invention features nucleic acid molecules of at least 525, 550, 600, 650, 700, 750, 800, 850, 900, 950, or 969 nucleotides of the nucleotide sequence of SEQ ID NO:55, the nucleotide sequence of a human TANGO 261 cDNA, the nucleotide sequence of the human TANGO 261 cDNA clone of ATCC® Accession No. 207176, or a complement thereof. The invention also features nucleic acid molecules comprising at least 280, 300, 320, 340, 360, 380, 400, 420, 440, 450 nucleotides of nucleic acids 1 to 453 of SEQ ID NO:55, or a complement thereof.

The invention features nucleic acid molecules of at least 560, 575, 600, 625, 650, 675, 700, 725, 750, 800, 850, 900, 950, 1000, 1100, 1200, 1300, 1400, 1500, 1600, 1700, or 1713 nucleotides of the nucleotide sequence of SEQ ID NO:57, the nucleotide sequence of a mouse TANGO 261 cDNA, or a complement thereof. The invention features nucleic acid molecules comprising at least 25 or 30 nucleotides of nucleic acids 1 to 33 of SEQ ID NO:57, or a complement thereof. The invention features nucleic acid molecules comprising at least 25, 30, 35, 40, 45, 50, 55, 60, 65, 70, 75, 80, 85, 90, 95, 100, 105, 110, 115, 120, 125, 130, 135, 140, 145, 150, 155, 160, 165, or 170 nucleotides of nucleic acids 550 to 725 of SEQ ID NO:57, or a complement thereof. The invention features nucleic acid molecules comprising at least 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 230, 235, 240, 245, 250, 255, 260, 265, 270, 275, 280, 285, 290, 295, or 300 nucleotides of nucleic acids 1404 to 1713 of SEQ ID NO:57, or a complement thereof.

The invention features nucleic acid molecules comprising at least 420, 425, 450, 475, 500, 525, 550, 600, or 650 nucleotides of the nucleotide sequence of the open reading frame of SEQ ID NO:57, or a complement thereof. The invention also features nucleic acid molecules comprising at least 25, 30, 35, 40, 45, 50, 55 or 60 nucleotides of nucleic acids 1 to 132, or of nucleic acids 549 to 651, of the open reading frame of SEQ ID NO:57, or a complement thereof.

The invention features nucleic acid molecules which include a fragment of at least 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, or 1682 nucleotides of the nucleotide sequence of SEQ ID NO:59, the nucleotide sequence of the human TANGO 262 cDNA clone of ATCC® Accession No. 207176, or a complement thereof. The invention features nucleic acid molecules comprising at least 25, 50, 100, 150, 200, 250, 300, 350, 400, or 440 nucleotides of nucleic acids 1 to 441 of SEQ ID NO:59, or a complement thereof. The invention also features nucleic acid molecules comprising at least 25, 50, 100, 150, 200, 250, 300, 350, 400, 450, 500, 525, or 530 nucleotides of nucleic acids 795 to 1329 of SEQ ID NO:59, the nucleotide sequence of the human TANGO 262 cDNA clone of ATCC® Accession No. 207176, or a complement thereof.

The invention features nucleic acid molecules of at least 355, 340, 350, 375, 400, 425, 450, 475, 500, 525, 550, 575, 600, 625, 650, or 677 nucleotides of the nucleotide sequence of the open reading frame of SEQ ID NO:59, the nucleotide sequence of a human TANGO 262 cDNA, or a complement thereof. The invention also features nucleic acid molecules comprising at least 25, 30, 40, 50, 60, 70, 80, 90, 100, 105, 110 or 115 nucleotides of nucleic acids 1 to 120 of the open reading frame of SEQ ID NO:59, or a complement thereof. The invention also features nucleic acid molecules comprising at least 25, 50, 75, 100, 125, 150, 175, or 200 nucleotides of nucleic acids 474 to 678 of the open reading frame of SEQ ID NO:59, or a complement thereof.

The invention features nucleic acid molecules of at least 340, 350, 400, 450, 500, 550, 600, 650, 700, 750, 800, 850, 900, 950, 1000, 1050, 1100, 1150, 1200, 1250, 1300, 1350, 1400 or 1422 nucleotides of the nucleotide sequence of SEQ ID NO:63, the nucleotide sequence of the human TANGO 266 cDNA clone of ATCC® Accession No. 207176, or a complement thereof. The invention also features nucleic acid molecules comprising at least 25, 50, 100, 125, 150, 175, 200, 225, 250, 275, 300, 325, 350, 375, 400, 425, 450, 475, 500, or 510 nucleotides of nucleic acids 1 to 520 of SEQ ID NO:63, or a complement thereof.

The invention features nucleic acid molecules of at least 590, 600, 650, 700, 750, 800, 850, 900, 950, 1000, 1100, 1200, 1300, 1400, 1500, 1600, 1700, 1800, 1900, 2000, 2100, 2200, 2250, 2300, 2350, 2400, 2450, 2500, 2550, 2600, 2650, 2700, 2750, 2800, 2850, 2900, or 2925 nucleotides of the nucleotide sequence of SEQ ID NO:63, the nucleotide sequence of the human TANGO 266 cDNA clone of ATCC® Accession No. 207176, or a complement thereof. The invention also features nucleic acid molecules comprising at least 25, 50, 100, 125, 150, 175, 200, 225, 250, 275, 300, 325, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1375, 1400, 1425, 1450, 1475, 1500, 1525, 1550, 1575, 1600, 1625, 1650, 1675, 1700, 1725, 1750, 1775, 1800, 1825, 1850, 1875, 1900, or 1925 nucleotides of nucleic acids 1 to 1940 of SEQ ID NO:63, or a complement thereof.

The invention features nucleic acid molecules which include a fragment of at least 590, 600, 650, 700, 750, 800, 850, 900, 950, 1000, 1100, 1200, 1300, 1400, 1500, 1600, 1700, 1800, 1900, 2000, 2100, 2200, 2250, 2300, or 2333 nucleotides of the nucleotide sequence of the open reading frame of human TANGO 266 of SEQ ID NO:63, or a complement thereof. The invention also features nucleic acid molecules comprising at least 25, 50, 100, 125, 150, 175, 200, 225, 250, 275, 300, 325, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1375, 1400, 1425, 1450, 1475, 1500, 1525, 1550, 1575, 1600, 1625, 1650, 1675, 1700, 1725, 1750, or 1775 nucleotides of nucleic acids 1 to 1780 of the open reading frame of human TANGO 266 of SEQ ID NO:63, or a complement thereof.

The invention features nucleic acid molecules of at least 480, 500, 550, 600, 650, 700, 750, 800, 850, 1000, 1100, 1200, 1300, 1400, 1500, 1600, 1700, 1800, 1900, 2000, 2100, 2200, 2300, 2400, 2500, 2600 or 2700 contiguous nucleotides of the nucleotide sequence of SEQ ID NO: 125, the nucleotide sequence of an EpT339 cDNA of ATCC® Accession Number PTA-292, or a complement thereof. The invention also features nucleic acid molecules comprising at least 20, 50, 100, 150, 200, 250, 300, 400, 450, 500, 550, 600, 650, 700, 750, 800, 850, 1000, 1100, 1200, 1300, 1400, 1500, 1600, 1700, 1800, 1900, 2000, 2100 contiguous nucleotides of nucleic acids 1 to 2102 of SEQ ID NO: 125, or a complement thereof.

The invention also features nucleic acid molecules that hybridize under stringent conditions to a nucleic acid molecule having the nucleotide sequence of cDNA or ORF of TANGO 339, or an EpT339 cDNA of ATCC® Accession Number PTA-292, or a complement thereof. In one embodiment, the nucleic acid molecules are at least 480, 500, 550, 600, 650, 700, 750, 800, 1000, 1100, 1200, 1300, 1400, 1500, 1600, 1600, 1700, 1800, 1900, 2000, 2100, 2200, 2300, 2400, 2500, 2600 or 2700 contiguous nucleotides in length and hybridize under stringent conditions to a nucleic acid molecule comprising the nucleotide sequence of TANGO 339, an EpT339 cDNA of ATCC® Accession Number PTA-292, or a complement thereof. In another embodiment, the nucleic acid molecules are at least 20, 50, 100, 150, 200, 250, 300, 350, 400, 450, 500, 550, 600, 650, 700, 750, 800, 900 or 1000 contiguous nucleotides in length and hybridize under stringent conditions to a nucleic acid molecule comprising the nucleotide sequence of TANGO 339 or nucleic acids 1 to 2100 of SEQ ID NO:125, or a complement thereof.

The invention also features nucleic acid molecules that hybridize under stringent conditions to a nucleic acid molecule having the nucleotide sequence of TANGO 383, or an EpT383 cDNA of ATCC® Accession Number PTA-295, or a complement thereof. In one embodiment, the nucleic acid molecules are at least 20, 50, 100, 150, 200, 250, 300, 350, 400, 450, 500, 550, or 600 contiguous nucleotides in length and hybridize under stringent conditions to a nucleic acid molecule comprising the nucleotides of SEQ ID NO: 135, or an EpT383 cDNA of ATCCE Accession Number PTA-295, or a complement thereof. Preferably, such nucleic acids hybridize under these conditions to at least a portion of nucleotides 1 to 250 and/or 800 to 1386 of SEQ ID NO:135.

The invention features nucleic acid molecules which are at least 80%, 85%, 90%, 95%, or 98% identical to the nucleotide sequence of SEQ ID NO: 147, the nucleotide sequence of the cDNA insert of an EpT499 clone deposited Aug. 5, 1999 with the ATCC® as Accession Number PTA-455, or a complement thereof. The invention features nucleic acid molecules which are at least 75%, 80%, 85%, 90%, 95%, or 98% identical to the nucleotide sequence of SEQ ID NO:147, or a complement thereof. The invention features nucleic acid molecules which are at least 30%, 35%, 40%, 45%, 50% 55%, 65%, 75% 85%, 95%, or 98% identical to the nucleotides 301 to 480 of SEQ ID NO: 147, or a complement thereof.

The invention features nucleic acid molecules which are at least 80%, 85%, 90%, 95%, or 98% identical to the nucleotide sequence of SEQ ID NO: 149, the nucleotide sequence of the cDNA insert of an EpT499 clone deposited Aug. 5, 1999 with the ATCC® as Accession Number PTA-454, or a complement thereof. The invention features nucleic acid molecules which are at least 75%, 80%, 85%, 90%, 95%, or 98% identical to the nucleotide sequence of SEQ ID NO: 149 or a complement thereof. The invention features nucleic acid molecules which are at least 30%, 35%, 40%, 45%, 50% 55%, 65%, 75%, 85%, 95%, or 98% identical to the nucleotides 240 to 344 of SEQ ID NO: 149 or a complement thereof.

The invention features nucleic acid molecules of at least 550, 600, 650, 700, 750, 800, 850, 1000, 1100, 1200, 1300, 1400, 1500, 1600, 1700, 1800, 1900, 2000 or 2020 contiguous nucleotides of the nucleotide sequence of SEQ ID NO: 141, the nucleotide sequence of an INT307 cDNA of ATCC® Accession Number PTA-455, or a complement thereof. The invention features nucleic acid molecules comprising at least 25, 50, 100, 150, 200, 250, 300, 350, 400, 450, 500, 550, 600 or 645 contiguous nucleotides of nucleic acids 1 to 649 of SEQ ID NO:141, or a complement thereof. The invention features nucleic acid molecules comprising at least 25, 50, 100, 150, 200, 250 or 300 contiguous nucleotides of nucleic acids 1120 to 1430 of SEQ ID NO:141, or a complement thereof.

The invention features nucleic acid molecules comprising at least 475, 500, 550, 600, 650, 700, 750, 800, 850, 900, 950, 1000, 1050 or 1085 contiguous nucleotides of nucleic acids 1 to 1086 of the open reading frame of SEQ ID NO: 141, or a complement thereof. The invention also features nucleic acid molecules comprising at least 25, 50, 100, 150, 200, 250, 300, 350, 400, 450, 500, 550 or 600 contiguous nucleotides of nucleic acids 1 to 604 of the open reading frame of SEQ ID NO: 141, or a complement thereof.

The invention features nucleic acid molecules of at least 350, 400, 450, 500, 550, 600, 650, 700, 750, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 contiguous nucleotides of the nucleotide sequence of SEQ ID NO: 145, the nucleotide sequence of an EpT361 cDNA of ATCC® Accession Number PTA-438, or a complement thereof. The invention also features nucleic acid molecules comprising at least 25, 50, 100, 150, 200, 250, 300, 400, 450, 500, 550, 600, 650, 700, 750, 800, 850, 1000, 1050, 1150 or 1170 contiguous nucleotides of nucleic acids 1 to 1176 of SEQ ID NO: 145, or a complement thereof. The invention features nucleic acid molecules comprising at least 25, 50, 100, 150 or 165 contiguous nucleotides of nucleic acids 1653 to 1821 of SEQ ID NO: 145, or a complement thereof. The invention features nucleic acid molecules comprising at least 25, 50, 100, 150, 200, 250, 300, 400, 450, 500, 550, 600, 650, 700, 750, 800, 850, 1000, 1100, 1200, 1300, 1400 or 1450 contiguous nucleotides of nucleic acids 2035 to 3506 of SEQ ID NO: 145, or a complement thereof. The invention also features nucleic acid molecules comprising at least 25, 50, 100, 150, 200, 250, 300, 400, 450, 500, 550, 600, 650, 700, 750, 800, 850, 1000, 1100, 1200, 1300, 1400, 1450 or 1490 contiguous nucleotides of nucleic acids 3564 to 5058 of SEQ ID NO:145, or a complement thereof.

The invention features nucleic acid molecules which include a fragment of at least 135, 150, 200, 250, 300, 400, 450, 500, 550, 600, 650, 700, 750, 800, 850, 900, 950, 1000, 1050, 1100, 1150, 1200 or 1250 contiguous nucleotides of the nucleotide sequence of the open reading frame of SEQ ID NO: 145, or a complement thereof. The invention features nucleic acid molecules which include a fragment of at least 25, 50, 100, 150, 200, 250, 300, 400, 450, 500, 550, 600, 650, 700, 750, 800, 850, 900, 950, 1000, 1050, 1100 or 1130 contiguous nucleotides of nucleic acids 1 to 1136 of the open reading frame of SEQ ID NO:145, or a complement thereof.

The invention features nucleic acid molecules of at least 500, 525, 550, 600, 650, 700, 750, 800, 850, 1000, or 1100 contiguous nucleotides of the nucleotide sequence of SEQ ID NO:147, the nucleotide sequence of an EpT499 cDNA of ATCC® Accession Number PTA-455, or a complement thereof. The invention also features nucleic acid molecules comprising at least 25, 50, 100, 150, or 174 contiguous nucleotides of nucleic acids 385 to 559 of SEQ ID NO: 147, or a complement thereof. The invention also features nucleic acid molecules comprising at least 25 contiguous nucleotides of nucleic acids 1072 to 1106 of SEQ ID NO:147, or a complement thereof.

The invention features nucleic acid molecules which include a fragment of at least 285, 300, 350, 400, 450, 500, 550, 600, 650, 700, 750 or 760 contiguous nucleotides of the nucleotide sequence of the open reading frame of SEQ ID NO: 147, or a complement thereof. The invention features nucleic acid molecules which include a fragment of at least 25, 50, 100, 150 or 175 contiguous nucleotides of nucleic acids 301 to 480 of the open reading frame of SEQ ID NO:147, or a complement thereof.

The invention features nucleic acid molecules of at least 500, 525, 550, 600, 650, 700, 750, 800, 850, 900, 950, 1000, 1050 or 1075 contiguous nucleotides of the nucleotide sequence of SEQ ID NO: 149, the nucleotide sequence of an EpT499 cDNA of ATCC® Accession Number PTA-454, or a complement thereof. The invention also features nucleic acid molecules comprising at least 25, 30, 50, 100, 150 or 175 contiguous nucleotides of nucleic acids 310 to 488 of SEQ ID NO: 149, or a complement thereof.

The invention also features nucleic acid molecules that hybridize under stringent conditions to a nucleic acid molecule having the nucleotide sequence of SEQ ID NO: 141 or an INT307 cDNA of ATCC® Accession Number PTA-455, or a complement thereof. In one embodiment, the nucleic acid molecules are at least 550, 600, 650, 700, 750, 800, 850, 1000, 1100, 1200, 1300, 1400, 1500, 1600, 1700, 1800, 1900, 2000 or 2020 contiguous nucleotides in length and hybridize under stringent conditions to a nucleic acid molecule comprising the nucleotide sequence of SEQ ID NO: 141, an INT307 cDNA of ATCC® Accession Number PTA-455, or a complement thereof. In another embodiment, the nucleic acid molecules are at least 25, 50, 100, 150, 200, 250, 300, 350, 400, 450, 500, 550, 600 or 645 contiguous nucleotides in length and hybridize under stringent conditions to a nucleic acid molecule comprising nucleic acids 1 to 649 of SEQ ID NO: 141, or a complement thereof. In another embodiment, the nucleic acid molecules are at least 25, 50, 100, 150, 200, 250 or 300 contiguous nucleotides in length and hybridize under stringent conditions to a nucleic acid molecule comprising nucleic acids 1120 to 1430 of SEQ ID NO: 141, or a complement thereof.

In another embodiment, the nucleic acid molecules are at least 475, 500, 550, 600, 650, 700, 750, 800, 850, 1000, 1050 or 1085 contiguous nucleotides in length and hybridize under stringent conditions to a nucleic acid molecule comprising the nucleotide sequence of the open reading frame of SEQ ID NO:141, or a complement thereof. In another embodiment, the nucleic acid molecules are at least 25, 50, 100, 150, 200, 250, 300, 350, 400, 450, 500, 550 or 600 contiguous nucleotides in length and hybridize under stringent conditions to a nucleic acid molecule comprising nucleic acids 1 to 604 of the open reading frame of SEQ ID NO: 141, or a complement thereof.

The invention also features nucleic acid molecules that hybridize under stringent conditions to a nucleic acid molecule having the nucleotide sequence of SEQ ID NO:145 or an EpT361 cDNA of ATCC® Accession Number PTA-438, or a complement thereof. In one embodiment, the nucleic acid molecules are at least 350, 400, 450, 500, 550, 600, 650, 700, 750, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 contiguous nucleotides in length and hybridize under stringent conditions to a nucleic acid molecule comprising the nucleotide sequence of TANGO 361, an EpT361 cDNA of ATCC® Accession Number PTA-438, or a complement thereof. In another embodiment, the nucleic acid molecules are at least 25, 50, 100, 150, 200, 250, 300, 350, 400, 450, 500, 550, 600, 650, 700, 750, 800, 900, 1000, 1100, 1150 or 1170 contiguous nucleotides in length and hybridize under stringent conditions to a nucleic acid molecule comprising nucleic acids 1 to 1176 of SEQ ID NO:145, or a complement thereof. In another embodiment, the nucleic acid molecules are at least 25, 50, 100, 150 or 165 contiguous nucleotides in length and hybridize under stringent conditions to a nucleic acid molecule comprising nucleic acids 1653 to 1821 of SEQ ID NO:145, or a complement thereof. In another embodiment, the nucleic acid molecules are at least 25, 50, 100, 150, 200, 250, 300, 350, 400, 450, 500, 550, 600, 650, 700, 750, 800, 900, 1000, 1100, 1200, 1300, 1400 or 1450 contiguous nucleotides in length and hybridize under stringent conditions to a nucleic acid molecule comprising nucleic acids 2035 to 3506 of SEQ ID NO:145, or a complement thereof. In another embodiment, the nucleic acid molecules are at least 25, 50, 100, 150, 200, 250, 300, 350, 400, 450, 500, 550, 600, 650, 700, 750, 800, 900, 1000, 1100, 1200, 1300, 1400, 1450 or 1490 contiguous nucleotides in length and hybridize under stringent conditions to a nucleic acid molecule comprising nucleic acids 3564 to 5058 of SEQ ID NO: 145, or a complement thereof.

In another embodiment, the nucleic acid molecules are at least 135, 150, 200, 250, 300, 350, 400, 450, 500, 550, 600, 650, 700, 750, 800, 900, 1000, 1100, 1200 or 1250 contiguous nucleotides in length and hybridize under stringent conditions to a nucleic acid molecule comprising the nucleotide sequence of the open reading frame of SEQ ID NO: 145, or a complement thereof. In yet another embodiment, the nucleic acid molecules are at least 25, 50, 100, 150, 200, 250, 300, 350, 400, 450, 500, 550, 600, 650, 700, 750, 800, 900, 1000, 1100 or 1130 contiguous nucleotides in length and hybridize under stringent conditions to a nucleic acid molecule comprising nucleic acids 1 to 1136 of the open reading frame of SEQ ID NO:145, or a complement thereof.

The invention also features nucleic acid molecules that hybridize under stringent conditions to a nucleic acid molecule having the nucleotide sequence of SEQ ID NO:147, or an EpT499 form 1, variant 1 cDNA of ATCC® Accession Number PTA-455, or a complement thereof. In one embodiment, the nucleic acid molecules are at least 500, 550, 600, 650, 700, 750, 800, 850, 1000 or 1100 contiguous nucleotides in length and hybridize under stringent conditions to a nucleic acid molecule comprising the nucleotide sequence of SEQ ID NO: 147, an EpT499 cDNA of ATCC® Accession Number PTA-455, or a complement thereof. In another embodiment, the nucleic acid molecules are at least 25, 50, 100, 150 or 175 contiguous nucleotides in length and hybridize under stringent conditions to a nucleic acid molecule comprising nucleic acids 385 to 563 of SEQ ID NO:147 or a complement thereof. In another embodiment, the nucleic acid molecules are at least 20 or contiguous nucleotides in length and hybridize under stringent conditions to a nucleic acid molecule comprising nucleic acids 1072 to 1106 of SEQ ID NO: 147, or a complement thereof.

In another embodiment, the nucleic acid molecules are at least 285, 300, 350, 400, 450, 500, 550, 600, 650, 700, 750 or 760 contiguous nucleotides in length and hybridize under stringent conditions to a nucleic acid molecule comprising the nucleotide sequence of the open reading frame of SEQ ID NO: 147, or a complement thereof. In yet another embodiment, the nucleic acid molecules are at least 25, 50, 100, 150 or 175 contiguous nucleotides in length and hybridize under stringent conditions to a nucleic acid molecule comprising nucleic acids 301 to 480 of the open reading frame of SEQ ID NO: 147, or a complement thereof.

The invention also features nucleic acid molecules that hybridize under stringent conditions to a nucleic acid molecule having the nucleotide sequence of SEQ ID NO: 149, or an EpT499 form 2, variant 3 cDNA of ATCC® Accession Number PTA-454, or a complement thereof. In one embodiment, the nucleic acid molecules are at least 500, 550, 600, 650, 700, 750, 800, 850, 1000, 1050 or 1075 contiguous nucleotides in length and hybridize under stringent conditions to a nucleic acid molecule comprising the nucleotide sequence of SEQ ID NO: 149, an EpT499 cDNA of ATCC® Accession Number PTA-454, or a complement thereof.

In another embodiment, the nucleic acid molecules are at least 275, 300, 350, 400, 450, 500, 550, 600, 650 or 675 contiguous nucleotides in length and hybridize under stringent conditions to a nucleic acid molecule comprising the nucleotide sequence of the open reading frame of SEQ ID NO: 149, or a complement thereof. In another embodiment, the nucleic acid molecules are at least 25, 50, 100, 150 or 175 contiguous nucleotides in length and hybridize under stringent conditions to a nucleic acid molecule comprising nucleic acids 240 to 344 of the open reading frame of SEQ ID NO: 149, or a complement thereof.

The invention features nucleic acid molecules of at least 700, 750, 800, 850, 1000, 1050, 1100, 1150, 1200, 1250, 1300, 1350, 1400 or 1450 contiguous nucleotides of the nucleotide sequence of SEQ ID NO:151, the nucleotide sequence of an EpT315 cDNA of ATCC® PTA-816, or a complement thereof. The invention features nucleic acid molecules comprising at least 25 30, 35, 40 or 45 contiguous nucleotides of nucleic acids 682 to 730 of SEQ ID NO:151, or a complement thereof.

The invention features nucleic acid molecules comprising at least 480, 500, 550, 600, 650, 700, 750, 800, 850 or 880 contiguous nucleotides of the nucleotide sequence of the open reading frame of SEQ ID NO: 151, or a complement thereof. The invention also features nucleic acid molecules comprising at least 25, 30, 35, 40 or 45 contiguous nucleotides of nucleic acids 682 to 730 of the open reading frame of SEQ ID NO: 151, or a complement thereof.

The invention features nucleic acid molecules comprising at least 480, 500, 550, 600, 650, 700, 750, 800 or 820 contiguous nucleotides of the nucleotide sequence of the open reading frame of SEQ ID NO: 153, or a complement thereof. The invention also features nucleic acid molecules comprising at least 25, 30, 35, 40 or 45 contiguous nucleotides of nucleic acids 625 to 673 of the open reading frame of SEQ ID NO: 153, or a complement thereof.

The invention features nucleic acid molecules of at least 626, 650, 700, 750, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000 or 3042 contiguous nucleotides of the nucleotide sequence of SEQ ID NO: 155, the nucleotide sequence of a clone 330a cDNA of ATCC® PTA-816, or a complement thereof. The invention features nucleic acid molecules comprising at least 25, 50, 100, 150, 200, 250, 300, 350, 400, 450, 500, 550, 600, 650, 700 or 720 contiguous nucleotides of nucleic acids 1090 to 1811 of SEQ ID NO: 155, or a complement thereof. The invention features nucleic acid molecules comprising at least 25, 50, 100, 150, 200, 250, or 260 contiguous nucleotides of nucleic acids 2782 to 3042 of SEQ ID NO:155, or a complement thereof.

The invention features nucleic acid molecules comprising at least 626, 650, 700, 750, 800, 850 or 880 contiguous nucleotides of the nucleotide sequence of the open reading frame of SEQ ID NO: 155, or a complement thereof. The invention features nucleic acid molecules comprising at least 25, 50, 100, 150, 200, 250, 300, 350, 400, 450, 500, 550, 600, 650, 700 or 720 contiguous nucleotides of nucleic acids 1088 to 1809 of the open reading frame of SEQ ID NO: 155, or a complement thereof.

The invention features nucleic acid molecules of at least 751, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800 or 3807 contiguous nucleotides of the nucleotide sequence of SEQ ID NO: 157, the nucleotide sequence of a clone 330b cDNA of ATCC® PTA-816, or a complement thereof. The invention features nucleic acid molecules comprising at least 25, 50, 100, 150, or 169 contiguous nucleotides of nucleic acids 1 to 150 of SEQ ID NO: 157, or a complement thereof. The invention features nucleic acid molecules comprising at least 25, 50, 100, 150, 5200, 250, 250, 300, 350, 400, 450, 500, 550, 600, 650, 700, 750, 800, 850, 900, 950, 1000 or 1034 contiguous nucleotides of nucleic acids 1090 to 2142 of SEQ ID NO:157, or a complement thereof. The invention features nucleic acid molecules comprising at least 25, 50, 100, 150 or 199 contiguous nucleotides of nucleic acids 2523 to 2723 of SEQ ID NO:157.

The invention features nucleic acid molecules comprising at least 751, 800, 850 or 880 contiguous nucleotides of the nucleotide sequence of the open reading frame of SEQ ID NO: 157, or a complement thereof. The invention features nucleic acid molecules comprising at least 25, 50, 100, 150, or 160 contiguous nucleotides of nucleic acids 1 to 140 of the open reading frame of SEQ ID NO: 157, or a complement thereof. The invention features nucleic acid molecules comprising at least 25, 50, 100, 150, 200, 250, 300, 350, 400 or 440 contiguous nucleotides of nucleic acids 1080 to 1439 of the open reading frame of SEQ ID NO:157, or a complement thereof.

The invention features nucleic acid molecules of at least 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300 or 4336 contiguous nucleotides of the nucleotide sequence of SEQ ID NO: 159, the nucleotide sequence of a clone 437 cDNA or a complement thereof. The invention also features nucleic acid molecules comprising at least 25, 50, 100, 150, 200, 250, 300, 350 or 380 contiguous nucleotides of nucleic acids 1 to 385 of SEQ ID NO: 159, or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1050, 1100, 1150 or 1200 contiguous nucleotides of nucleic acids 776 to 1976 of SEQ ID NO: 159, or a complement thereof. The invention also features nucleic acid molecules comprising at least 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400 or 1445 contiguous nucleotides of nucleic acids 2889 to 4336 of SEQ ID NO:159, or a complement thereof.

The invention features nucleic acid molecules which include a fragment of at least 390, 400, 450, 500, 550, 600, 650, 700, 750, 800, 850, 900, 950, 1000, 1100, 1200, 1300, 1400, 1500, 1600, 1700, 1750 or 1770 contiguous nucleotides of the nucleotide sequence of the open reading frame of SEQ ID NO:159, or a complement thereof. The invention also features nucleic acid molecules comprising at least 25, 50, 100, 150, 200, 250, 300, 350 or 385 contiguous nucleotides of nucleic acids 1 to 385 of the open reading frame of SEQ ID NO:159, or a complement thereof. The invention also features nucleic acid molecules comprising at least 25, 50, 100, 150, 200, 250, 300, 350, 400, 450, 500, 550, 600, 650, 700, 750, 800, 850, 900, 950 or 997 contiguous nucleotides of nucleic acids 776 to 1773 of the open reading frame of SEQ ID NO:159, or a complement thereof.

The invention features nucleic acid molecules of at least 565, 600, 650, 700, 750, 800, 850, 900, 950, 1000, 1100, 1200, 1300, 1400, 1500, 1600, 1700, 1800, or 1912 contiguous nucleotides of the nucleotide sequence of SEQ ID NO:161, the nucleotide sequence of a clone 480 cDNA or a complement thereof. The invention also features nucleic acid molecules comprising at least 25, 50, 100, 150, 200, 250, 300, 350, 400, 450, 500, 550, 600, 650, 700, 750, 800, or 835 contiguous nucleotides of nucleic acids 1 to 835 of SEQ ID NO:161, or a complement thereof. The invention also features nucleic acid molecules comprising at least 25, 50, 100 or 112 contiguous nucleotides of nucleic acids 1231 to 1344 of SEQ ID NO:161, or a complement thereof.

The invention features nucleic acid molecules of at least 25, 50, 75, 100, 150, 200, 250, 300, 350, 400, 450, 500, 550 or 579 contiguous nucleotides of the nucleotide sequence of the open reading frame of SEQ ID NO:161, the nucleotide sequence of a clone 480 cDNA or a complement thereof.

The invention also features nucleic acid molecules that hybridize under stringent conditions to a nucleic acid molecule having the nucleotide sequence of TANGO 315 or an EpT315 cDNA of ATCC® deposit number PTA-816, or a complement thereof. In one embodiment, the nucleic acid molecules are at least 700, 750, 800, 850, 1000, 1050, 1100, 1150, 1200, 1250, 1300, 1350, 1400 or 1450 contiguous nucleotides in length and hybridize under stringent conditions to a nucleic acid molecule comprising the nucleotide sequence of SEQ ID NO: 151, an EpT315 cDNA of ATCC® deposit number PTA-816, or a complement thereof. In another embodiment, the nucleic acid molecules are at least 25, 30, 35, 40 or 45 contiguous nucleotides in length and hybridize under stringent conditions to a nucleic acid molecule comprising nucleic acids 682 to 730 of SEQ ID NO: 151, or a complement thereof.

In another embodiment, the nucleic acid molecules are at least 480, 500, 550, 600, 650, 700, 750, 800, 850 or 880 contiguous nucleotides in length and hybridize under stringent conditions to a nucleic acid molecule comprising the nucleotide sequence of the open reading frame of SEQ ID NO: 151, or a complement thereof. In another embodiment, the nucleic acid molecules are at least 25, 30, 35, 40, 50, 100, 150 or 195 contiguous nucleotides in length and hybridize under stringent conditions to a nucleic acid molecule comprising nucleic acids 682 to 730 of the open reading frame of SEQ ID NO: 151, or a complement thereof.

In another embodiment, the nucleic acid molecules are at least 480, 500, 550, 600, 650, 700, 750, 800, 850 or 860 contiguous nucleotides in length and hybridize under stringent conditions to a nucleic acid molecule comprising the nucleotide sequence of the open reading frame of SEQ ID NO:153, or a complement thereof. In another embodiment, the nucleic acid molecules are at least 25, 30, 35, 40 or 45 contiguous nucleotides in length and hybridize under stringent conditions to a nucleic acid molecule comprising nucleic acids 625 to 673 of the open reading frame of SEQ ID NO: 153, or a complement thereof.

The invention also features nucleic acid molecules that hybridize under stringent conditions to a nucleic acid molecule having the nucleotide sequence of the cDNA of TANGO 330 or a Clone 330a cDNA of ATCC® deposit number PTA-816, or a complement thereof. In one embodiment, the nucleic acid molecules are at least 626, 650, 700, 750, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000 or 3042 contiguous nucleotides in length and hybridize under stringent conditions to a nucleic acid molecule comprising the nucleotide sequence of SEQ ID NO:155, a clone 330a cDNA of ATCC® deposit number PTA-816, or a complement thereof. In another embodiment, the nucleic acid molecules are at least 25, 50, 100, 150, 200, 250, 300, 350, 400, 450, 500, 550, 600, 650, 700 or 720 contiguous nucleotides in length and hybridize under stringent conditions to a nucleic acid molecule comprising nucleic acids 1090 to 1811 of SEQ ID NO: 155, or a complement thereof. In another embodiment, the nucleic acid molecules are at least 25, 50, 100, 150, 200 or 260 contiguous nucleotides in length and hybridize under stringent conditions to a nucleic acid molecule comprising nucleic acids 2782 to 3042 of SEQ ID NO: 155, or a complement thereof.

In another embodiment, the nucleic acid molecules are at least 626, 650, 700, 750, 800, 850, 1000, 1050, 1100, 1200, 1300, 1400, 1500, 1600, 1700, 1800, 1900, 2000, 2100, 2200, 2300, 2400, 2500, 2600, 2700 or 2802 contiguous nucleotides in length and hybridize under stringent conditions to a nucleic acid molecule comprising the nucleotide sequence of the open reading frame of SEQ ID NO:155, or a complement thereof. In another embodiment, the nucleic acid molecules are at least 25, 50, 100, 150, 200, 250, 300, 350, 400, 450, 500, 550, 600, 650, 700 or 720 contiguous nucleotides in length and hybridize under stringent conditions to a nucleic acid molecule comprising nucleic acids 1088 to 1809 of the open reading frame of SEQ ID NO: 155, or a complement thereof.

The invention also features nucleic acid molecules that hybridize under stringent conditions to a nucleic acid molecule having the nucleotide sequence of SEQ ID NO: 157, a clone 330b cDNA of ATCC® deposit number PTA-816, or a complement thereof. In one embodiment, the nucleic acid molecules are at least 751, 800, 850, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800 or 3807 contiguous nucleotides in length and hybridize under stringent conditions to a nucleic acid molecule comprising the nucleotide sequence of SEQ ID NO: 157, a clone 330b cDNA of ATCC® deposit number PTA-816, or a complement thereof. In another embodiment, the nucleic acid molecules are at least 25, 50, 100, 150 or 169 contiguous nucleotides in length and hybridize under stringent conditions to a nucleic acid molecule comprising nucleic acids 1 to 150 of SEQ ID NO:157, or a complement thereof. In another embodiment, the nucleic acid molecules are at least 25, 50, 100, 150, 200, 250, 300, 350, 400, 450, 500, 550, 600, 650, 700, 750, 800, 850, 900, 950, 1000 or 1034 contiguous nucleotides in length and hybridize under stringent conditions to a nucleic acid molecule comprising nucleic acids 1090 to 2142 of SEQ ID NO:157, or a complement thereof. In another embodiment, the nucleic acid molecules are at least 25, 50, 100, 150 or 199 contiguous nucleotides in length and hybridize under stringent conditions to a nucleic acid molecule comprising nucleic acids 2523 to 2723 of SEQ ID NO: 157, or a complement thereof.

In another embodiment, the nucleic acid molecules are at least 751, 800, 850, 1000, 1050, 1100, 1200, 1300, 1400 or 1440 contiguous nucleotides in length and hybridize under stringent conditions to a nucleic acid molecule comprising the nucleotide sequence of the open reading frame of SEQ ID NO: 157, or a complement thereof. In another embodiment, the nucleic acid molecules are at least 25, 50, 100, 150 or 160 contiguous nucleotides in length and hybridize under stringent conditions to a nucleic acid molecule comprising nucleic acids 1 to 140 of the open reading frame of SEQ ID NO: 157, or a complement thereof. In another embodiment, the nucleic acid molecules are at least 25, 50, 100, 150, 200, 250, 300, 350, 400, or 440 contiguous nucleotides in length and hybridize under stringent conditions to a nucleic acid molecule comprising nucleic acids 1080 to 1439 of the open reading frame of SEQ ID NO:157, or a complement thereof.

In another embodiment, the nucleic acid molecules are at least 25, 50, 100, 150, 200, 250, 300, 350 or 380 contiguous nucleotides in length and hybridize under stringent conditions to a nucleic acid molecule comprising nucleic acids 1 to 385 of TANGO 437, or a complement thereof. In another embodiment, the nucleic acid molecules are at least 25, 50, 100, 150, 200, 250, 300, 350, 400, 450, 500, 550, 600, 650, 700, 750, 800, 850, 900, 950, 1000, 1050, 1100, 1150 or 1200 contiguous nucleotides in length and hybridize under stringent conditions to a nucleic acid molecule comprising nucleic acids 776 to 1976 of SEQ ID NO: 159, or a complement thereof. In another embodiment, the nucleic acid molecules are at least 25, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400 or 1445 contiguous nucleotides in length and hybridize under stringent conditions to a nucleic acid molecule comprising nucleic acids 2887 to 4336 of SEQ ID NO:159, or a complement thereof.

The invention also features nucleic acid molecules that hybridize under stringent conditions to a nucleic acid molecule having the nucleotide sequence of TANGO 437-form 2, or a complement thereof. In one embodiment, the nucleic acid molecules are at least 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, or 3700 contiguous nucleotides in length and hybridize under stringent conditions to a nucleic acid molecule comprising the nucleotide sequence of TANGO 437-form 2, or a complement thereof. In another embodiment, the nucleic acid molecules are at least 390, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2210, or 2220 contiguous nucleotides in length and hybridize under stringent conditions to a nucleic acid molecule comprising the nucleotide sequence of the ORF of TANGO 437-form 2, or a complement thereof.

In another embodiment, the nucleic acid molecules are at least 390, 400, 450, 500, 550, 600, 650, 700, 750, 800, 850, 900, 950, 1000, 1100, 1200, 1300, 1400, 1500, 1600, 1700, 1750 or 1770 contiguous nucleotides in length and hybridize under stringent conditions to a nucleic acid molecule comprising the nucleotide sequence of the open reading frame of SEQ ID NO:159, or a complement thereof. In another embodiment, the nucleic acid molecules are at least 25, 50, 100, 150, 200, 250, 300 or 340 contiguous nucleotides in length and hybridize under stringent conditions to a nucleic acid molecule comprising nucleic acids 1 to 385 of the open reading frame of SEQ ID NO: 159, or a complement thereof. In another embodiment, the nucleic acid molecules are at least 25, 50, 100, 150, 200, 250, 300, 350, 400, 450, 500, 550, 600, 650, 700, 750, 800, 850, 900, 950 or 990 contiguous nucleotides in length and hybridize under stringent conditions to a nucleic acid molecule comprising nucleic acids 776 to 1773 of the open reading frame of SEQ ID NO: 159, or a complement thereof.

In another embodiment, the nucleic acid molecules are at least 25, 50, 100, 150, 200, 250, 300, 350, 400, 450, 500, 550, 600, 650, 700, 750, 800, or 830 contiguous nucleotides of in length and hybridize under stringent conditions to a nucleic acid molecule comprising nucleic acids 1 to 835 of SEQ ID NO:161, or a complement thereof. In another embodiment, the nucleic acid molecules are at least 25, 50, 100, or 113 contiguous nucleotides in length and hybridize under stringent conditions to a nucleic acid molecule comprising nucleic acids 1231 to 1344 of SEQ ID NO: 161, or a complement thereof.

In preferred embodiments, the isolated nucleic acid molecules encode a cytoplasmic, transmembrane, or extracellular domain of a polypeptide of the invention.

In one embodiment, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention, or modulators thereof. In another embodiment, the invention provides host cells containing such a vector or engineered to contain and/or express a nucleic acid molecule of the invention. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention, or modulators thereof. Preferred proteins and polypeptides possess at least one biological activity possessed by the corresponding naturally-occurring human polypeptide. An activity, a biological activity, or a functional activity of a polypeptide or nucleic acid of the invention refers to an activity exerted by a protein, polypeptide or nucleic acid molecule of the invention on a responsive cell as determined in vivo or in vitro, according to standard techniques. Such activities can be a direct activity, such as an association with or an enzymatic activity on a second protein, or an indirect activity, such as a cellular signaling activity mediated by interaction of the protein with a second protein.

Another aspect of this invention features isolated or recombinant proteins and polypeptides of the invention, or modulators thereof. Preferred proteins and polypeptides possess at least one biological activity possessed by the corresponding naturally-occurring human polypeptide. An activity, a biological activity, and a functional activity of a polypeptide of the invention refers to an activity exerted by a protein or polypeptide of the invention on a responsive cell as determined in vivo, or in vitro, according to standard techniques. Such activities can be a direct activity, such as an association with or an enzymatic activity on a second protein or an indirect activity, such as a cellular signaling activity mediated by interaction of the protein with a second protein. Thus, such activities include, e.g., (1) the ability to form protein-protein interactions with proteins in the signaling pathway of the naturally-occurring polypeptide; (2) the ability to bind a ligand of the naturally-occurring polypeptide; (3) the ability to bind to an intracelluar target of the naturally-occurring polypeptide.

Further activities of polypeptides of the invention include the ability to modulate (this term, as used herein, includes, but is not limited to, “stabilize”, promote, inhibit or disrupt, protein-protein interactions (e.g., homophilic and/or heterophilic)), protein-ligand interactions, e.g., in receptor-ligand recognition, development, differentiation, maturation, proliferation and/or activity of cells function, survival, morphology, proliferation and/or differentiation of cells of tissues in which it is expressed. Additional activities include but are not limited to: (1) the ability to modulate cell surface recognition; (2) the ability to transduce an extracellular signal (e.g., by interacting with a ligand and/or a cell-surface receptor); (3) the ability to modulate a signal transduction pathway; and (4) the ability to modulate intracellular signaling cascades (e.g., signal transduction cascades).

Other activities of polypeptides of the invention may include, e.g., (1) the ability to modulate cellular proliferation; (2) the ability to modulate cellular differentiation; (3) the ability to modulate chemotaxis and/or migration; and (4) the ability to modulate cell death.

For HtrA-2 (TANGO 214) or modulators thereof, additional biological activities include, e.g., (1) the ability to modulate growth factor function, e.g., that of insulin-like growth factor (IGF), e.g., IGF-I, IGF-II, by, for example, modulating the availability of growth factors and/or their receptors; (2) the ability to modulate (e.g., inhibit) the activity of a proteolytic enzyme, e.g., a serine protease; and (3) the ability to modulate the function, migration, proliferation (e.g., suppress cell growth), and/or differentiation of cells, e.g., cells in tissues in which it is expressed (see description of expression data below) and, in particular, bone cells such as osteoblasts and osteoclasts, and cartilage cells such as chondrocytes.

Other activities of HtrA-2 or modulators thereof include: (1) the ability to act as a proteolytic enzyme cleaving either itself (e.g., autocatalysis, e.g., autocatalysis between its own Kazal and serine protease domains) or other substrates; (2) the ability to bind to an inhibitor of proteolytic enzyme activity, e.g., an inhibitor of a serine protease, e.g., α₁-antitrypsin; (3) the ability to modulate the activity of proteins (e.g., TGF-beta family members) in the activin/inhibin growth factor system; and (4) the ability to perform one or more of the functions of human HtrA described, for example, in Hu et. al. (1998) J. Biol. Chem. 273(51):34406-34412, the contents of which are incorporated herein by reference.

Other activities of HtrA-2 or modulators thereof include: (1) the ability to modulate the function of a normal or mutated presenilin protein (e.g., presenilin-1 (PS-1) or presenilin-2 (PS-2)); and (2) the ability to perform a function of the human serine protease PSP-1, described in EP 828 003, the contents of which are incorporated herein by reference.

Still other activities of HtrA-2 or modulators thereof include: (1) the ability to modulate protein degradation, e.g., degradation of denatured and/or misfolded proteins; (2) the ability to act as a chaperone protein, e.g., to renature misfolded proteins and help to restore their function; (3) the ability to interact with (e.g., bind to) the normal or mutated gene product of a human presenilin gene (e.g., human presenilin 1 (PS-1), e.g., mutant PS-1 TM16TM2 loop domain as described in PCT Publication Number WO 98/01549, published Jan. 15, 1998); (4) the ability to interact with (e.g., bind to) a protein expressed in brain; (5) the ability to modulate a neurological function; (6) the ability to interact with (e.g., bind to) a protein containing the following consensus sequence: Xaa-Ser/Thr-Xaa-Val-COO—, where Xaa is any amino acid, Ser is Serine, Thr is Threonine, Val is Valine (which can be substituted with other hydrophobic residues), and COO— is the protein C terminus; and (7) the ability to modulate production and secretion of prostaglandin.

For TANGO 221 or modulators thereof, additional biological activities include, e.g., the ability to interact with a TANGO 221 receptor. Other activities include the ability to modulate function, survival, morphology, proliferation and/or differentiation of cells of tissues in which it is expressed (e.g., cells of adipose tissue, breast tissue, and fetal liver and spleen tissues). With regard to adipose tissue, examples of biological activities of TANGO 221 include the ability to modulate synthesis, storage, and release of lipids, and to modulate the conversion of stored chemical energy into heat.

For TANGO 222 or modulators thereof, additional biological activities include, e.g., the ability to interact with a TANGO 222 receptor. Other activities include: (1) the ability to modulate function, survival, morphology, proliferation and/or differentiation of cells of tissues in which it is expressed (e.g., cells of adipose tissue). In adipose tissue, for example, TANGO 222 biological activities include the ability to modulate synthesis, storage, and release of lipids, and to modulate the conversion of stored chemical energy into heat.

For TANGO 176 or modulators thereof, additional biological activities include, e.g., (1) the ability to interact with a TANGO 176 receptor; (2) the ability to act as a serine carboxypeptidase, e.g., act as a serine carboxypeptidase at an acidic lysosomal pH (e.g., between pH 2 and pH 6); (3) the ability to act as a deamidase, e.g., act as a deamidase at a neutral pH (e.g., between pH 7 and pH 7.5); and (4) the ability to perform a function of cathepsin A. Other activities include the ability to modulate function, survival, morphology, proliferation and/or differentiation of cells of tissues in which it is expressed (e.g., cells of the pituitary gland).

For TANGO 201 or modulators thereof, additional biological activities include, e.g., the ability to interact with a TANGO 201 receptor. Other activities include (1) the ability to modulate function, survival, morphology, proliferation and/or differentiation of cells of tissues in which it is expressed (e.g., pancreas, adrenal medulla, thyroid, adrenal cortex, testis, stomach, heart, brain, placenta, lung, liver, kidney, skeletal muscle, or small intestine); and (2) the ability to function in the amplification of cellular oncogenes.

For TANGO 223 or modulators thereof, additional biological activities include, e.g., the ability to interact with a TANGO 223 receptor. Other activities include the ability to modulate function, survival, morphology, proliferation and/or differentiation of cells of tissues in which it is expressed (e.g., heart, brain, liver, kidney, testis, prostate, ovary, colon, peripheral blood leukocytes, and the small intestine).

For TANGO 253 or modulators thereof, additional biological activities include, e.g., (1) the ability to modulate the development, differentiation, maturation, proliferation and/or activity of cells of the central nervous system such as neurons, glial cells (e.g., astrocytes and oligodendrocytes), and Schwann cells; (2) the ability to modulate the development of central nervous system; (3) the ability to modulate the development, differentiation, maturation, proliferation and/or activity of renal cells; (4) the ability to modulate the development, differentiation, maturation, proliferation and/or activity of testicle cells, such as germ cells, leydig cells and Sertoli cells; (5) the ability to modulate the development, differentiation, maturation, proliferation and/or activity of ovarian cells; (6) ability to modulate cell-cell interactions and/or cell-extracellular matrix interactions; (7) the ability to modulate the host immune response, e.g., by modulating one or more elements in the serum complement cascade; (8) the ability to modulate the proliferation, differentiation and/or activity of cells that form blood vessels and coronary tissue (e.g., coronary smooth muscle cells and/or blood vessel endothelial cells); and (9) the ability to modulate adipocyte function.

For TANGO 257 or modulators thereof, additional biological activities include, e.g., (1) the ability to modulate the development, differentiation, proliferation and/or activity of neuronal cells, e.g., olfactory neurons (2) the ability to modulate the development, differentiation, proliferation and/or activity of pulmonary system cells, e.g., lung cell types; (4) the ability to modulate the development, differentiation, maturation, proliferation and/or activity of bone cells such as osteocytes, osteoblasts and osteoclasts (e.g., the ability promote the development of osteocytes); (5) the ability to modulate the development of bone structures such as the skull, the basisphenoid bone, the upper and lower incisor teeth, the vertebral column, the sternum, the scapula, and the femur during embryogenesis; (6) the ability to modulate the development, differentiation, maturation, proliferation and/or activity of renal cells; (7) the ability to modulate the development, differentiation, maturation, proliferation and/or activity of intestinal cells such as M cells; (8) the ability to modulate cell-cell interactions and/or cell-extracellular matrix interactions, e.g., neuronal cell-extracellular matrix interactions; and (9) the ability to modulate the development, differentiation, proliferation and/or activity of cells that form blood vessels and coronary tissue, e.g., coronary smooth muscle cells and/or blood vessel endothelial cells.

For INTERCEPT 258 or modulators thereof, additional biological activities include, e.g., (1) the ability to modulate the host immune response; (2) the ability to modulate the development, differentiation, maturation, proliferation and/or activity of pulmonary system cells such as bronchial cells; (3) the ability to modulate the development, differentiation, maturation, proliferation and/or activity of renal cells; (4) the ability to modulate the development, differentiation, maturation, proliferation and/or activity of cardiac cells such cardiac myocytes; (5) the ability to modulate the development of brown fat (e.g., the promotion of the development of brown fat); (6) the ability to modulate the development, differentiation, maturation, proliferation and/or activity of endothelial cells; (7) the ability to modulate cell proliferation, e.g., gastrointestinal tract epithelial cell proliferation; and (8) the ability to modulate thrombosis (e.g., the ability to facilitate the removal of blood clots) and/or vascularization (e.g., the promotion of vascularization).

For TANGO 204 or modulators thereof, additional biological activities include, e.g., the ability to interact with a TANGO 204 receptor. TANGO 204 biological activities can include the ability to act as a protease inhibitor.

For TANGO 206 or modulators thereof, additional biological activities include, e.g., the ability to interact with a TANGO 206 receptor. TANGO 206 biological activities can include the ability to modulate cell migration and acid secretion by gastric mucosal tissue.

For TANGO 209 or modulators thereof, additional biological activities include, e.g., the ability to interact with a TANGO 209 receptor. Other activities include the ability to modulate function, survival, morphology, proliferation and/or differentiation of cells of tissues in which it is expressed (e.g., cells of the pituitary gland). TANGO 209 biological activities can include the ability to modulate the availability of growth factors, the ability to modulate cell migration, and the ability to modulate embryonic growth.

For TANGO 244 or modulators thereof, additional biological activities include, e.g., the ability to interact with a TANGO 244 receptor. Other activities include the ability to modulate function, survival, morphology, proliferation and/or differentiation of cells of tissues in which it is expressed.

For TANGO 246 or modulators thereof, additional biological activities include, e.g., the ability to interact with a TANGO 246 receptor. TANGO 246 biological activities can include the ability to act as a small molecule transporter or a cell cycle regulator.

For TANGO 275 or modulators thereof, additional biological activities include, e.g., the ability to interact with a TANGO 275 receptor. Other activities include the ability to modulate function, survival, morphology, proliferation and/or differentiation of cells of tissues in which it is expressed (e.g., cells of the pituitary gland). TANGO 275 biological activities can include: (1) the ability to act as a TGF-β binding protein; (2) the ability to facilitate the normal assembly and secretion of large latent complexes containing TGF-β; (3) the ability to target latent TGF-β to connective tissue; (4) the ability to target latent TGF-β to the cell surface; (5) the ability to modulate bone formation, renewal, or remodeling; and (6) the ability to modulate the development or function of the heart, cardiovascular system, brain, placenta, liver, skeletal muscle, kidney or pancreas.

For MANGO 245 or modulators thereof, additional biological activities include, e.g., the ability to interact with a MANGO 245 receptor. Other activities include the ability to modulate function, survival, morphology, proliferation and/or differentiation of cells of tissues in which it is expressed, e.g., the central nervous system, and the ability to modulate the cellular functions of cells of the nervous system (neurons and glial cells), and the ability to act as a modulator of complement function.

For INTERCEPT 340 or modulators thereof, additional biological activities include, e.g., (1) the ability to interact with an INTERCEPT 340 receptor, e.g., a cell surface receptor (e.g., an integrin); (2) the ability to modulate the activity of an intracellular molecule that participates in a signal transduction pathway, e.g., an intracellular molecule in the integrin signaling (e.g., a cdk2 inhibitor); (3) the ability to assemble into fibrils; (4) the ability to strengthen and organize the extracellular matrix; (5) the ability to modulate the shape of tissues and cells; (6) the ability to interact with (e.g., bind to) components of the extracellular matrix; and (7) the ability to modulate cell migration. Other activities include the ability to modulate function, survival, morphology, migration, proliferation and/or differentiation of cells of tissues in which it is expressed (e.g., splenic cells). For example, additional biological activities of INTERCEPT 340 include: (1) the ability to modulate splenic cell activity; (2) the ability to modulate skeletal morphogenesis; and/or (3) the ability to modulate smooth muscle cell proliferation and differentiation.

For MANGO 003 or modulators thereof, additional biological activities include, e.g., (1) the ability to interact with a MANGO 003 receptor, e.g., a cell surface receptor; and (2) the ability to modulate signal transmission at a chemical synapse. Other activities include the ability to modulate function, survival, morphology, proliferation and/or differentiation of cells of tissues in which it is expressed (e.g., thyroid, liver, skeletal muscle, kidney, heart, lung, testis and brain). For example, the activities of MANGO 003 can include modulation of endocrine, hepatic, skeletal muscular, renal, cardiovascular, reproductive and/or brain function.

For MANGO 347 or modulators thereof, additional biological activities include, e.g., (1) the ability to interact with a MANGO 347 receptor; and (2) the ability to modulate a developmental process, e.g., morphogenesis, cellular migration, adhesion, proliferation, differentiation, and/or survival. Other activities include the ability to modulate function, survival, morphology, proliferation and/or differentiation of cells of tissues in which it is expressed (e.g., brain cells). For example, the activities of MANGO 347 can include modulation of neural (e.g., CNS) function.

For TANGO 272 or modulators thereof, additional biological activities include, e.g., (1) the ability to interact with a TANGO 272 receptor, e.g., a cell surface receptor (e.g. an integrin); (2) the ability to modulate cell attachment; (3) the ability to modulate cell fate; and (4) the ability to modulate tissue repair and/or wound healing. Other activities include the ability to modulate function, survival, morphology, proliferation and/or differentiation of cells of tissues in which it is expressed (e.g., micro vascular endothelial cells). For example, the activities of TANGO 272 can include modulation of cardiovascular function.

For TANGO 295 or modulators thereof, additional biological activities include, e.g., (1) the ability to interact with (e.g., bind to) a nucleic acid; and (2) the ability to elicit pyrimidine-specific endonuclease activity. Other activities include the ability to modulate function, survival, morphology, proliferation and/or differentiation of cells of tissues in which it is expressed (e.g., mammary epithelium).

For TANGO 354 or modulators thereof, additional biological activities include, e.g., the ability to modulate function, survival, morphology, proliferation and/or differentiation of cells of tissues in which it is expressed (e.g., hematopoietic tissues). For example, TANGO 354 biological activities can further include: (1) regulation of hematopoietic; (2) modulation (e.g., increasing or decreasing) of homeostasis; (3) modulation of an inflammatory response; (4) modulation of neoplastic growth, e.g., inhibition of tumor growth; and (5) modulation of thrombolysis.

For TANGO 378 or modulators thereof, additional biological activities include, e.g., the ability to modulate a signal transduction pathway (e.g., adenylate cyclase, or phosphatidylinositol 4,5-bisphosphate (PIP₂), inositol 1,4,5-triphosphate (IP₃)). Other activities include the ability to modulate function, survival, morphology, proliferation and/or differentiation of cells of tissues in which it is expressed (e.g., natural killer cells). For example, TANGO 378 biological activities can further include the ability to modulate an immune response in a subject, for example, (1) by modulating immune cytotoxic responses against pathogenic organisms, e.g., viruses, bacteria, and parasites; (2) by modulating organ rejection after transplantation; and (3) by modulating immune recognition and lysis of normal and malignant cells.

For TANGO 339 or modulators thereof, additional biological activities include, e.g., (1) the ability to modulate the development, differentiation, proliferation and/or activity of immune cells (e.g., B-lymphocyte function); (2) the ability to modulate the development and progression of cancer (e.g. lymphomas and/or melanoma-associated cancer); (3) the ability to modulate hematopoietic processes; (4) the ability to modulate platelet activation and aggregation; (5) the ability to modulate intercellular signaling (e.g., in the nervous system); (6) the ability modulate the development, differentiation, proliferation and/or activity of neuronal cells and glial cells (e.g., oligodendrocytes and astrocytes); (7) the ability to modulate the development, differentiation and activity of eye structures, such as the retina (e.g., the ability to modulate photoreceptor disk morphogenesis); and (8) the ability to modulate the development of organs, tissues and/or cells in an embryo and/or fetus.

For TANGO 358 or modulators thereof, additional biological activities include, e.g., (1) the ability to modulate development, differentiation, maturation, proliferation and/or activity of immune cells such as thymocytes, e.g., T-lymphocytes; (2) the ability to modulate the host immune response; and (3) the ability to modulate intercellular signaling (e.g., in the immune system).

For TANGO 365 or modulators thereof, additional biological activities include, e.g., the ability to modulate, protein-protein interactions (e.g., homophilic and/or heterophilic), and protein-ligand interactions, e.g., in TANGO 365 receptor-ligand recognition.

For TANGO 368 or modulators thereof, additional biological activities include, e.g., the ability to modulate, protein-protein interactions (e.g., homophilic and/or heterophilic), and protein-ligand interactions, e.g., in TANGO 368 receptor-ligand recognition.

For TANGO 369 or modulators thereof, additional biological activities include, e.g., (1) the ability to modulate development, differentiation, proliferation and/or activity of cells, such as immune cells, e.g., natural killer cells; (2) the ability to modulate the host immune response; (3) the ability to modulate intercellular signaling (e.g., in the immune system); and (4) the ability to modulate, protein-protein interactions (e.g., homophilic and/or heterophilic), and protein-ligand interactions, e.g., in TANGO 369 receptor-ligand recognition.

For TANGO 383 or modulators thereof, additional biological activities include, e.g., ability to modulate cell-cell interactions and/or cell-extracellular matrix interactions.

For MANGO 346 or modulators thereof, additional biological activities include, e.g., ability to modulate cell-cell interactions and/or cell-extracellular matrix interactions.

For MANGO 349 or modulators thereof, additional biological activities include, e.g., (1) the ability to modulate the proliferation, differentiation and/or activity of neural cells; and (2) the ability to modulate intracellular signaling cascades (e.g., signal transduction cascades).

For INTERCEPT 307 or modulators thereof, additional biological activities include, e.g., (1) the ability to modulate the development, differentiation, morphology, migration or chemotaxis, proliferation and/or activity of immune cells (e.g., T-lymphocyte function); (2) the ability to modulate the development and progression of cell proliferative disorders such as cancer (e.g., prostate cancer); (3) the ability to modulate hematopoietic processes; (4) the ability to modulate the proliferation, differentiation, and/or function of prostate cells; (5) the ability to modulate infections, e.g., infections mediated by eosinophil granule release; and (6) the ability to modulate the function, e.g., activation, of eosinophils.

For MANGO 511 or modulators thereof, additional biological activities include, e.g., (1) the ability to modulate the development, differentiation, morphology, migration or chemotaxis, proliferation and/or activity of immune cells (e.g., B-lymphocytes and monocytes); (2) the ability to modulate hematopoietic processes; (3) the ability to modulate MHC class I recognition and binding; (4) the ability to modulate ligand-receptor interactions in proteins with immunoglobulin domains; (5) the ability to modulate immunoglobulin binding to antigens; and (6) the ability to modulate lymphocyte selection (such as modulation of B-cell receptor or T-cell receptor stimulation in developing lymphocytes, e.g., through modulation of antigen interaction with immunoglobulin domains of the receptors).

For TANGO 361 or modulators thereof, additional biological activities include, e.g., (1) the ability to modulate the development, differentiation, morphology, migration or chemotaxis, proliferation and/or activity of prostate cells (e.g. prostate epithelial cells) or adipocytes; (2) the ability to modulate the development and progression of cell proliferative disorders such as cancer (e.g. prostate or prostate-associated cancer); (3) the ability to act as a protease (e.g., serine protease) and/or modulate protease (e.g., serine protease) activities, such as serine protease activity involved in platelet function, (e.g., activation and aggregation), serine protease activity involved in progression of Alzheimer's disease (e.g., formation of Alzheimer's plaques), or serine protease activity involved in activation of the complement system (e.g., C3b cleavage); (4) the ability to modulate intercellular signaling (e.g., in the prostate); (5) the ability to modulate suppression of infectious diseases or cancer (e.g., bacteria, viruses, parasites, or neoplastic cells); (6) the ability to modulate autoimmunity (e.g., as associated with multiple sclerosis, psoriasis, arthritis, lupus); (7) the ability to modulate transplant rejections (e.g., graft rejections, or allograft rejections); (8) the ability to modulate carbohydrate binding; and (9) the ability to modulate systemic energy balance.

For TANGO 499 or modulators thereof, additional biological activities include, e.g., (1) the ability to modulate the development, differentiation, morphology, migration or chemotaxis, proliferation, survival and/or activity of neurons, (e.g., peripheral neurons and/or central neurons), glial cells, (e.g., oligodendrocytes or astrocytes) or endocrine cells (e.g., pituitary cells or pineal gland cells); (2) the ability to modulate the development and progression of cell proliferative disorders such as cancer (e.g., glial associated cancers such as glioblastoma) or neural associated cancer; (3) the ability to modulate intercellular signaling (e.g., in the nervous system); (4) the ability to modulate the development of neural organs and tissues; (5) the ability to modulate riboflavin-delivery to the embryo; and (6) the ability to modulate the development of an embryo and/or fetal development.

For TANGO 315 or modulators thereof, additional biological activities include, e.g., (1) the ability to track and/or modulate the development, differentiation, morphology, migration or chemotaxis, proliferation and/or activity of immune cells (e.g., natural killer cell function); (2) the ability to modulate the development and progression of cell proliferative disorders such as cancer (e.g., myeloid leukemia); (3) the ability to track and/or modulate hematopoietic processes; (4) the ability to track and/or modulate the development, proliferation, activity and function of adipocytes; (5) the ability to track and/or modulate neuroendrocrine function and activity, e.g., neuroendrocrine secretion; (6) the ability to modulate energy metabolism; (7) the ability to modulate appetite (e.g., obesity or cachexia); and (8) the ability to track and/or modulate embryonic development.

For TANGO 330 or modulators thereof, additional biological activities include, e.g., (1) the ability to track and/or modulate the development, differentiation, morphology, migration or chemotaxis, proliferation, survival, activity and/or function of neurons, (e.g., peripheral neurons and/or central neurons); (2) the ability to track and/or modulate the development, differentiation, morphology, migration or chemotaxis, proliferation, survival, activity and/or function of glial cells, (e.g., oligodendrocytes or astrocytes); (3) the ability to track and/or modulate the development, differentiation, morphology, migration or chemotaxis, proliferation, survival, activity and/or function of endocrine cells (e.g., adrenal gland cells neural organs or tissues or endocrine organs or tissues); (4) the ability to track and/or modulate intercellular signaling (e.g., in the nervous system); and (5) the ability to track and/or modulate cell cycle progression.

For TANGO 437 or modulators thereof, additional biological activities include, e.g., (1) the ability to track and/or modulate the development, differentiation, morphology, migration or chemotaxis, proliferation, activity and/or function of immune cells (e.g., B cells, T cells and monocytes); (2) the ability to track and/or modulate hematopoietic processes; and (3) the ability to track and/or modulate ion transport (e.g., sodium, calcium or potassium transport).

For TANGO 480 or modulators thereof, additional biological activities include, e.g., the ability to track and/or modulate the development, differentiation, morphology, migration or chemotaxis, proliferation, activity and/or function of keratinocytes.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have or encode a common structural domain and/or common functional activity. For example, amino acid or nucleotide sequences which contain or encode a common structural domain having about 60% identity, preferably about 65% identity, more preferably about 75%, 85%, 95%, 98% or more identity are defined herein as sufficiently identical.

In one embodiment, the isolated polypeptides of the invention include at least one or more of the following domains: a signal sequence, an extracellular domain, a transmembrane domain and an intracellular or cytoplasmic domain.

In another embodiment, the isolated polypeptide of the invention lacks both a transmembrane and cytoplasmic domain. In yet another embodiment, a polypeptide of the invention lacks both a transmembrane and a cytoplasmic domain and is soluble under physiological conditions. In yet another embodiment, a polypeptide of the invention is fused to either heterologous sequences, or is fused in twco or more repeats of a domain, e.g., binding or enzymatic, and is soluble under physiological conditions.

The polypeptides of the present invention, or biologically active portions thereof, can be operably linked to a heterologous amino acid sequence to form fusion proteins. The invention further features antibody substances that specifically bind a polypeptide of the invention, such as monoclonal or polyclonal antibodies, antibody fragments, and single-chain antibodies. In addition, the polypeptides of the invention or biologically active portions thereof can be incorporated into pharmaceutical compositions, which optionally include pharmaceutically acceptable carriers. These antibody substances can be made, for example, by providing the polypeptide of the invention to an immuno-competent vertebrate and thereafter harvesting blood or serum from the vertebrate.

In another aspect, the present invention provides methods for detecting the presence, activity or expression of a polypeptide of the invention in a biological sample by contacting the biological sample with an agent capable of detecting an indicator of the presence, activity or expression such that the presence activity or expression of a polypeptide of the invention is detected in the biological sample.

In another aspect, the invention provides methods for modulating activity of a polypeptide of the invention comprising contacting a cell with an agent that modulates (e.g., inhibits or stimulates) the activity or expression of a polypeptide of the invention such that activity or expression in the cell is modulated. In one embodiment, the agent is an antibody that specifically binds to a polypeptide of the invention. In another embodiment, the agent is a fragment of a polypeptide of the invention or a nucleic acid molecule encoding such a polypeptide fragment.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense to the coding strand of an mRNA encoding a polypeptide of the invention.

The present invention also provides methods to treat a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention. In other embodiments, the modulator is a polypeptide (e.g., an antibody or a fragment of a polypeptide of the invention), a peptidomimetic, or other small molecule (e.g., a small organic molecule).

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of the invention wherein a wild-type form of the gene encodes a protein having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

Other features and advantages of the invention will be apparent from the following detailed description and claims.

### BRIEF DESCRIPTION OF THE DRAWINGS

FIG. 1A-1D depicts a partial cDNA sequence and predicted partial amino acid sequence of mouse TANGO 136 (SEQ ID NO: 2). The open reading frame extends from nucleotide 89 to nucleotide 1813 of SEQ ID NO: 1. In this and other sequence depictions described herein the open reading frame of the cDNA is indicated by nucleotide triplets, above which the amino acid sequence is listed.

FIG. 2 depicts a hydropathy plot of a portion of mouse TANGO 136. Relatively hydrophobic residues are above the horizontal line, and relatively hydrophilic residues are below the horizontal line. The cysteine residues (cys) and potential N-glycosylation sites (Ngly) are indicated by short vertical lines just below the hydropathy trace. A dashed vertical line separates the signal sequence on the left from the mature protein on the right.

FIG. 3A-3E depicts the cDNA sequence and predicted amino acid sequence of human TANGO 136 (SEQ ID NO: 4). The open reading frame of extends from nucleotide 541 to 2679 of SEQ ID NO: 3.

FIG. 4 depicts a hydropathy plot of human TANGO 136, the details of which are described herein.

FIG. 5A-5B depicts an alignment of the amino acid sequences of mouse TANGO 136 (partial sequence, human TANGO 136, human LRp105 and rat LRp105).

FIG. 6A-6E depicts an alignment of the nucleic acid sequences of mouse TANGO 136 (partial sequence) and human TANGO 136.

FIG. 7A-7B depicts an alignment of the amino acid sequences of mouse TANGO 136 (partial sequence; upper sequence) and human TANGO 136 (lower sequence).

FIG. 8 depicts alignments of the CUB-like domains of mouse TANGO 136 (lower sequence) with a consensus CUB domain (upper sequence). In these alignments an uppercase letter between the two sequences indicates an exact match, and a “+” indicates a similarity.

FIG. 9 depicts alignments of the CUB-like domains of human TANGO 136 (lower sequence) with a consensus CUB domain (upper sequence). In these alignments an uppercase letter between the two sequences indicates an exact match, and a “+” indicates a similarity.

FIG. 10 depicts alignments of the LDL class A domains of human TANGO 136 (lower sequence) with a consensus LDL class A domain (upper sequence). In these alignments an uppercase letter between the two sequences indicates an exact match, and a “+” indicates a similarity.

FIG. 11A-11D depicts the cDNA sequence of human TANGO 128 and predicted amino acid sequence of TANGO 128 (SEQ ID NO: 6). The open reading frame extends from nucleotide 288 to 1322 of SEQ ID NO: 5.

FIG. 12A-12B depicts the cDNA sequence of human TANGO 140-1 and predicted amino acid sequence of TANGO 140-1 (SEQ ID NO: 8). The open reading frame extends from nucleotide 2 to 622 of SEQ ID NO: 7.

FIG. 13A-13C depicts the cDNA sequence of human TANGO 140-2 and predicted amino acid sequence of TANGO 140-2 (SEQ ID NO: 10). The open reading frame extends from nucleotide 1 to 594 of SEQ ID NO: 9.

FIG. 14A-14C depicts the cDNA sequence of human TANGO 197 and predicted amino acid sequence of TANGO 197 (SEQ ID NO: 12). The open reading frame extends from nucleotide 213 to 1211 of SEQ ID NO: 11.

FIG. 15A-15E depicts the cDNA sequence of human TANGO 212 and predicted amino acid sequence of TANGO 212 (SEQ ID NO: 14). The open reading frame extends from nucleotide 269 to 1927 of SEQ ID NO: 13.

FIG. 16A-16C depicts the cDNA sequence of human TANGO 213 and predicted amino acid sequence of TANGO 213 (SEQ ID NO: 16). The open reading frame extends from nucleotide 58 to 870 of SEQ ID NO: 15.

FIG. 17A-17D depicts the cDNA sequence of human TANGO 224 and predicted amino acid sequence of TANGO 224 (SEQ ID NO: 18). The open reading frame extends from nucleotide 1 to 1440 of SEQ ID NO: 17.

FIG. 18 depicts a hydropathy plot of a human TANGO-128, the details of which are described herein.

FIG. 19 depicts a hydropathy plot of a human TANGO 140-1, the details of which are described herein.

FIG. 20 depicts a hydropathy plot of a human TANGO 140-2, the details of which are described herein.

FIG. 21 depicts a hydropathy plot of a human TANGO 197, the details of which are described herein.

FIG. 22 depicts a hydropathy plot of a human TANGO 212, the details of which are described herein.

FIG. 23 depicts a hydropathy plot of a human TANGO 213, the details of which are described herein.

FIG. 24 depicts a hydropathy plot of a human TANGO 224, the details of which are described herein.

FIG. 25 depicts the alignment of amino acids 269 to 337 of TANGO 128 and the platelet derived growth factor (PDGF) consensus sequence. In these alignments, an uppercase letter between the two sequences indicates an exact match, and a (+) indicates a conservative amino acid substitution.

FIG. 26 depicts the alignment of amino acids 48 to 160 of TANGO 128 (amino acids 48 to 160 and the CUB consensus sequence. In these alignments, an uppercase letter between the two sequences indicates an exact match, and a (+) indicates a conservative amino acid substitution.

FIG. 27 depicts the alignment of amino acids 11 to 49 and amino acids 52 to 91 of TANGO 140-1 with the tumor necrosis factor receptor (TNF-R) consensus sequence. In these alignments, an uppercase letter between the two sequences indicates an exact match, and a (+) indicates a conservative amino acid substitution.

FIG. 28 depicts the alignment of amino acids 25 to 63 and amino acids 66 to 105 of TANGO 140-2 with the tumor necrosis factor receptor (TNF-R) consensus sequence. In these alignments, an uppercase letter between the two sequences indicates an exact match, and a (+) indicates a conservative amino acid substitution.

FIG. 29 depicts the alignment of amino acids 44 to 215 of TANGO 197 and the von Willebrand Factor (vWF) consensus sequence. In these alignments, an uppercase letter between the two sequences indicates an exact match, and a (+) indicates a conservative amino acid substitution.

FIG. 30 depicts the alignment of amino acids 61 to 91, amino acids 98 to 132, amino acids 138 to 172, amino acids 178 to 217, and amino acids 223 to 258 of TANGO 212 and the epidermal growth factor (EGF) consensus sequence. In these alignments, an uppercase letter between the two sequences indicates an exact match, and a (+) indicates a conservative amino acid substitution.

FIG. 31 depicts the alignment of amino acids 400 to 546 of TANGO 212 and the MAM consensus sequence. In these alignments, an uppercase letter between the two sequences indicates an exact match, and a (+) indicates a conservative amino acid substitution.

FIG. 32 depicts the alignment of amino acids 37 to 81 of TANGO 224 and the thrombospondin type-I (TSP-I) consensus sequence. In these alignments, an uppercase letter between the two sequences indicates an exact match, and a (+) indicates a conservative amino acid substitution.

FIG. 33A-33B depicts the cDNA sequence of mouse TANGO 128 and predicted amino acid sequence of mouse TANGO 128 (SEQ ID NO: 22). The open reading frame comprises from nucleotides 211 to 750 of SEQ ID NO: 21.

FIG. 34A-34D depicts the cDNA sequence of mouse TANGO 197 and predicted amino acid sequence of mouse TANGO 197 (SEQ ID NO: 24). The open reading frame extends from nucleotide 3 to 1145 of SEQ ID NO: 23.

FIG. 35A-35C depicts the cDNA sequence of mouse TANGO 212 and predicted amino acid sequence of mouse TANGO 212 (SEQ ID NO: 26). The open reading frame extends from nucleotide 180 to 1179 of SEQ ID NO: 25.

FIG. 36A-36C depicts the cDNA sequence of mouse TANGO 213 and predicted amino acid sequence of mouse TANGO 213 (SEQ ID NO: 28). The open reading frame extends from nucleotide 41 to 616 of SEQ ID NO: 27.

FIG. 37A-37F depicts the cDNA sequence of human TANGO 224, form 2 (clone Athsa25a8) and predicted amino acid sequence of human TANGO 224, form 2 (clone Athsa25a8). The open reading frame extends from nucleotide 67 to 2690.

FIG. 38 depicts the cDNA sequence of rat TANGO 213 (SEQ ID NO: 29).

FIG. 39A-39D depicts the cDNA sequence of human HtrA-2 and the predicted amino acid sequence of HtrA-2 (TANGO 214; SEQ ID NO: 32). The open reading frame extends from nucleotide 222 to nucleotide 1580 of SEQ ID NO: 31.

FIG. 40A-40B. FIG. 40A depicts a hydropathy plot of human HtrA-2, the details of which are described herein. FIG. 40B depicts the amino acid sequence of HtrA-2.

FIG. 41A-41H depicts an alignment of the nucleotide sequence of human HtrA (SEQ ID NO: 165; GenBank Accession Number Y07921) and the nucleotide sequence of human HtrA-2. The nucleotide sequences of human HtrA and human HtrA-2 are 50.9% identical. This alignment was performed using the ALIGN alignment program with a PAM120 scoring matrix.

FIG. 42A-42D depicts an alignment of the nucleotide sequence of the open reading frames of human HtrA (nucleotides 39 to 1478) and human HtrA-2. The nucleotide sequences of the open reading frames of human HtrA and human HtrA-2 are 62.3% identical. This alignment was performed using the ALIGN alignment program with a PAM120 scoring matrix.

FIG. 43A-43B depicts an alignment of the amino acid sequence of human HtrA and the amino acid sequence of human HtrA-2. The amino acid sequences of human HtrA and human HtrA-2 are 56.5% identical. This alignment was performed using the ALIGN alignment program with a PAM120 scoring matrix.

FIG. 44A-44C depicts the cDNA sequence of mouse HtrA-2 (TANGO 214) and the predicted amino acid sequence of HtrA-2 (SEQ ID NO: 34). The open reading frame extends from nucleotides 268 to 1311 of SEQ ID NO: 33.

FIG. 45 depicts the cDNA sequence and the predicted amino acid sequence of human TANGO 221 (SEQ ID NO: 36). The open reading frame extends from nucleotide 6 to nucleotide 716 of SEQ ID NO: 35.

FIG. 46 depicts a hydropathy plot of human TANGO 221, the details of which are described herein.

FIG. 47 depicts the cDNA sequence and the predicted amino acid sequence of human TANGO 222 (SEQ ID NO: 38). The open reading frame extends from nucleotide 33 to nucleotide 434 of SEQ ID NO: 37.

FIG. 48 depicts a hydropathy plot of human TANGO 222, the details of which are described herein.

FIG. 49A-49B depicts the cDNA sequence and the predicted ammo acid sequence of human TANGO 176 (SEQ ID NO: 40). The open reading frame extends from nucleotide 101 to nucleotide 1528 of SEQ ID NO: 39.

FIG. 50 depicts a hydropathy plot of human TANGO 176, the details of which are described herein.

FIG. 51A-51B depicts the cDNA sequence of mouse TANGO 176 and predicted amino acid sequence of mouse TANGO 176 (SEQ ID NO: 42). The open reading frame extends from nucleotide 49 to 1524 of SEQ ID NO: 41.

FIG. 52A-52C depicts the cDNA sequence and the predicted amino acid sequence of mouse TANGO 201 (SEQ ID NO: 44). The open reading frame extends from nucleotide 60 to nucleotide 1508 of SEQ ID NO: 43.

FIG. 53 depicts a hydropathy plot of mouse TANGO 201, the details of which are described herein.

FIG. 54A-54D depicts the cDNA sequence and the predicted amino acid sequence of human TANGO 201 (SEQ ID NO: 46). The open reading frame extends from nucleotide 179 to nucleotide 1387 of SEQ ID NO: 45.

FIG. 55 depicts a hydropathy plot of human TANGO 201, the details of which are described herein.

FIG. 56A-56D depicts an alignment of the nucleotide sequence of mouse TANGO 201 (nucleotides 1-1758) and human TANGO 201 (nucleotides 101-1660. An identity of 84.8% was obtained using the program GAP (Needleman and Wunsch (1970) J. Mol. Biol. 48:443-453) in GCG (Wisconsin Package Version 9.1, Genetics Computer Group, Madison Wis.) with the following settings: score matrix nwsgapdna, gap penalty 50, and gap extension penalty 3.

FIG. 57 depicts an alignment of the amino acid sequences of mouse TANGO 5201 (amino acids 1-483) and human TANGO 201 (amino acids 1403). An identity of 97% was obtained using the program GAP (Needleman and Wunsch (1970) J. Mol. Biol. 48:443-453) in GCG (Wisconsin Package Version 9.1, Genetics Computer Group, Madison Wis.) with the following settings: score matrix blosum62, gap penalty 12, and gap extension penalty 4.

FIG. 58 depicts an alignment of a portion of mouse TANGO 201 amino acid sequence (amino acids 78-264) and a portion of human TANGO 201 amino acid sequence (amino acids 78-264) with a portion of OS-9, a human protein referred to as OS-9 (amino acids 73-250 of SwissProt Accession No. Q13438; SEQ ID NO: 166). This alignment defines a cysteine-rich domain that is conserved between TANGO 201 and OS-9.

FIG. 59A-59B depicts the cDNA sequence and the predicted amino acid sequence of human TANGO 223 (SEQ ID NO: 48). The open reading frame of human TANGO 223 extends from nucleotide 30 to nucleotide 770 of SEQ ID NO: 47.

FIG. 60 depicts a hydropathy plot of human TANGO 223, the details of which are described herein.

FIG. 61 depicts an alignment of a portion of human TANGO 223 amino acid sequence (amino acids 82-180) with a portion of a putative C. elegans protein belonging to the family of DNA/RNA nonspecific endonucleases (amino acids 288-378 of Swiss-Prot Accession No. 001975; SEQ ID NO: 167). This alignment reveals a cysteine-rich domain that is conserved between TANGO 223 and the C. elegans protein.

FIG. 62A-62B depicts the cDNA sequence and the predicted amino acid sequence of mouse TANGO 223 (SEQ ID NO: 50). The open reading frame of mouse TANGO 223 extends from nucleotide 5 to nucleotide 694 of SEQ ID NO: 49.

FIG. 63A-63C depicts the cDNA sequence of human TANGO 216 and predicted amino acid sequence of human TANGO 216 (SEQ ID NO: 52). The open reading frame extends from nucleotide 307 to 1770 of SEQ ID NO: 51.

FIG. 64A-64C depicts the cDNA sequence of mouse TANGO 216 and predicted amino acid sequence of mouse TANGO 216 (SEQ ID NO: 54). The open reading frame extends from nucleotide 149 to 1609 of SEQ ID NO: 53.

FIG. 65 depicts a hydropathy plot of human TANGO 216, the details of which are described herein.

FIG. 66 depicts the alignment of the amino acid sequence of human TANGO 216 and mouse TANGO 216. In this alignment, a (|) between the two sequences indicates an exact match and a (:) indicates similarity.

FIG. 67 depicts the cDNA sequence of human TANGO 261 and predicted amino acid sequence of human TANGO 261 (SEQ ID NO: 56). The open reading frame extends from nucleotide 6 to 761 of SEQ ID NO: 55.

FIG. 68 depicts the cDNA sequence of a mouse TANGO 261 clone and predicted amino acid sequence of mouse TANGO 261 (SEQ ID NO: 58). The open reading frame extends from nucleotide 2 to 652 of SEQ ID NO: 57.

FIG. 69 depicts a hydropathy plot of human TANGO 261, the details of which are described herein.

FIG. 70 depicts the alignment of the amino acid sequence of human TANGO 261 and a portion of mouse TANGO 261. In this alignment, a (|) between the two sequences indicates an exact match.

FIG. 71A-71B depicts the cDNA sequence of human TANGO 262 and predicted amino acid sequence of human TANGO 262 (SEQ ID NO: 60). The open reading frame extends from nucleotide 322 to 999 of SEQ ID NO: 59.

FIG. 72A-72B depicts the cDNA sequence of mouse TANGO 262 and predicted amino acid sequence of mouse TANGO 262 (SEQ ID NO: 62). The open reading frame extends from nucleotide 89 to 766 of SEQ ID NO: 61.

FIG. 73 depicts a hydropathy plot of human TANGO 262, the details of which are described herein.

FIG. 74 depicts the alignment of the amino acid sequence of human TANGO 262 and mouse TANGO 262. In this alignment, a (|) between the two sequences indicates an exact match.

FIG. 75 depicts the alignment of the amino acid sequence of human TANGO 262 and KC3.4 (SEQ ID NO: 168). In this alignment, a (•) between the two sequences indicates an exact match.

FIG. 76 depicts the cDNA sequence of human TANGO 266 and predicted amino acid sequence of human TANGO 266 (SEQ ID NO: 64). The open reading frame of the human TANGO 266 cDNA extends from nucleotide 49 to 363 of SEQ ID NO: 63.

FIG. 77 depicts a hydropathy plot of a human TANGO 266, the details of which are described herein.

FIG. 78 depicts the alignment of the amino acid sequence of human TANGO 266 and Dendroaspis polypepis venom protein A (SwissProt Accession Number P25687; SEQ ID NO: 169. In this alignment, a (•) between the two sequences indicates an exact match.

FIG. 79A-79C depicts the cDNA sequence of human TANGO 267 and predicted amino acid sequence of human TANGO 267 (SEQ ID NO: 66). The open reading frame of human TANGO 267 extends from nucleotide 161 to 2494 of SEQ ID NO: 65.

FIG. 80A-80D depicts the alignment of the amino acid sequence of human TANGO 267 and hepatocellular carcinoma associated gene JCL-1 (GenBank Accession Number U92544; SEQ ID NO: 179. In this alignment, a (•) between the two sequences indicates an exact match.

FIG. 81A-81D. 81A: Amino acid sequence alignment of Mbkn (TANGO 266) with Bv8 and VPRA. Regions with significant identity are boxed. Numbers correspond to the sequence of the adjacent protein. mBv8-3 is a mouse splice variant 3 of Bv8, and fBv8 is frog Bv8. 81B: Schematic diagram with relative phylogenetic relationship between Mbkn, Bv8, and VPRA. 81C: Hydrophobicity profile and location of cysteines (cys) of Mbkn. The vertical line represents a signal peptide cleavage site. 81D: Western Blot analysis of recombinant MbknFc and MbknAP fusion proteins as well as supernatants from 293 cells and 3T3 cell supernatants using affinity purified rabbit anti-Mbkn polyclonal antibodies.

FIG. 82A-82B. 82A: Northern blot analysis of multiple human tissue RNAs hybridized with a Mbkn probe. 82B: Relative expression of Mbkn in multiple human tissues by quantitative PCR of cDNA. C: In situ expression of Mbkn detected in the ovarian stroma, but no expression was detected in the ovarian endothelium. Moderate expression detected in the placenta.

FIG. 83 depicts alkaline phosphatase detected on the surface of macrophages only in the presence of a Mbkn-AP (TANGO 266-alkaline phosphatase) fusion polypeptide, demonstrating that Mbkn-AP specifically binds to cultured mouse macrophages and is inhibited from binding by Mbkn-Fc fusion protein.

FIG. 84A-84B depicts the cDNA sequence of human TANGO 253 and the predicted amino acid sequence of human TANGO 253 (SEQ ID NO: 68). The open reading frame extends from nucleotide 188 to nucleotide 916 of SEQ ID NO: 67.

FIG. 85 depicts a hydropathy plot of human TANGO 253, the details of which are described herein. Below the hydropathy plot, the amino acid sequence of human TANGO 253 is depicted.

FIG. 86A-86B depicts a cDNA sequence of mouse TANGO 253 and the predicted amino acid sequences of mouse TANGO 253 (SEQ ID NO: 70). The open reading frame extends from nucleotide 135 to 863 of SEQ ID NO: 69.

FIG. 87 depicts a hydropathy plot of mouse TANGO 253, the details of which are described herein. Below the hydropathy plot, the amino acid sequence of mouse TANGO 253 is depicted.

FIG. 88 depicts an alignment of the amino acid sequence of human TANGO 253 and the amino acid sequence of mouse TANGO 253. The alignment demonstrates that the amino acid sequences of human and mouse TANGO 253 are 93.8% identical. This alignment was performed using the ALIGN program with a PAM120 scoring matrix, a gap length penalty of 12 and a gap penalty of 4.

FIG. 89A-89B depicts alignments of the amino acid sequence of human adipocyte complement-mediated protein precursor (Swiss Prot Accession Number Q 15848; SEQ ID NO: 171) and the amino acid sequence of human TANGO 253 (89A) or mouse TANGO 253 (89B). 89A shows the amino acid sequences of human adipocyte complement-mediated protein precursor and human TANGO 253 are 38.7% identical. 89B shows the amino acid sequences of human adipocyte complement-mediated precursor procursor protein and mouse TANGO 253 are 38.3% identical. These alignments were performed using the ALIGN alignment program with a PAM120 scoring matrix, a gap length penalty of 12, and a gap penalty of 4.

FIG. 90A-90D depicts alignments of the nucleotide sequence of human adipocyte complement-mediated protein precursor (GenBank Accession Number A1417523; SEQ ID NO: 172) and the nucleotide sequence of human TANGO 253. The nucleotide sequences of human adipocyte complement-mediated protein precursor and human TANGO 253 are 29.1% identical. These alignments were performed using the ALIGN alignment program with a PAM120 scoring matrix, a gap length penalty of 12, and a gap penalty of 4.

FIG. 91A-91D depicts alignments of the nucleotide sequence of human adipocyte complement-mediated protein precursor (GenBank Accession Number A1417523; SEQ ID NO: 172) and the nucleotide sequence of mouse TANGO 253. The nucleotide sequences of human adipocyte complement-mediated protein precursor and mouse TANGO 253 are 30.4% identical. These alignments were performed using the ALIGN alignment program with a PAM120 scoring matrix, a gap length penalty of 12, and a gap penalty of 4.

FIG. 92A-92C depicts the cDNA sequence of human TANGO 257 and the predicted amino acid sequence of human TANGO 257 (SEQ ID NO: 72). The open reading frame extends from nucleotide 88 to nucleotide 1305 of SEQ ID NO: 171.

FIG. 93 depicts a hydropathy plot of human TANGO 257, the details of which are described herein. Below the hydropathy plot, the amino acid sequence of human TANGO 257 is depicted.

FIG. 94A-94C depicts a cDNA sequence of mouse TANGO 257 and the predicted amino acid sequence of mouse TANGO 257 (SEQ ID NO: 74). The open reading frame extends from nucleotide 31 to 1248 of SEQ ID NO: 73.

FIG. 95 depicts a hydropathy plot of mouse TANGO 257, the details of which are described herein. Below the hydropathy plot, the amino acid sequence of mouse TANGO 257 is depicted.

FIG. 96 depicts an alignment of the amino acid sequence of human TANGO 257 and the amino acid sequence of mouse TANGO 257. This alignment demonstrates that the amino acid sequences of human and mouse TANGO 257 are 94.1% identical. This alignment was performed using the ALIGN program with a PAM120 scoring matrix, a gap length penalty of 12 and a gap penalty of 4.

FIG. 97 depicts an alignment of the amino acid sequence encoded by a nucleotide sequence referred to in PCT publication WO 98/39446 (SEQ ID NO: 173) as “gene 64”, and the amino acid sequence of human TANGO 257. Gene 64 encodes a 353 amino acid residue protein that exhibits homology with the human extracellular molecule olfactomedin, which is though to be involved in maintenance, growth and/or differentiation of chemosensory cilia on the apical dendrites of olfactory neurons. The polypeptide encoded by gene 64 also exhibits homology to human TANGO 257, which contains 406 amino acids (i.e., an additional 53 amino acids carboxy to residue 353). The amino acid sequences of amino acid residues 1-353 of the gene 64-encoded polypeptide and human TANGO 257 are identical. As such, the overall amino acid sequence identity between the full length polypeptide encoded by gene 64, and the full-length human TANGO 257 polypeptide is approximately 87%. This alignment was performed using the ALIGN alignment program with a PAM120 scoring matrix, a gap length penalty of 12, and a gap penalty of 4.

FIG. 98A-98D depicts an alignment of the nucleotide sequence of gene 64 (PCT Publication Number WO 98/39446 (Accession No. AC02146; SEQ ID NO: 173) and the nucleotide sequence of human TANGO 257. The nucleotide sequences of gene 64 and human TANGO 257 are 93.5% identical. It is noted, however, that among the differences between the two sequences is a cytosine nucleotide at human TANGO 257 position 1146 that results in a human TANGO 257 amino acid sequence of 406 amino acids as opposed to the gene 64 amino acid sequence of only 353 amino acids. Alignment of the nucleotide sequence of the gene 64 open reading frame and that of human TANGO 257 show that the two nucleotide sequences are 87.2% identical. These alignments were performed using the ALIGN program with a PAM220 scoring matrix, a gap length penalty of 12 and a gap penalty of 4.

FIG. 99 depicts an alignment of the amino acid sequence of the gene 64-encoded polypeptide and the amino acid sequence of mouse TANGO 257. The sequences exhibit an overall amino acid sequence identity of approximately 81.8%. This alignment was performed using an ALIGN program with a PAM120 scoring matrix, a gap length penalty of 12 and a gap penalty of 4.

FIG. 100A-100F depicts an alignment of the nucleotide sequence of gene 64 and the nucleotide sequence of mouse TANGO 257. The two sequences are approximately 76.2% identical. Alignment of the nucleotide sequence of the gene 64 open reading frame and that of mouse TANGO 257 shows that the two nucleotide sequences are 77.8% identical. These alignments were performed using the ALIGN program with a PAM220 scoring matrix, a gap length penalty of 12 and a gap penalty of 4.

FIG. 101A-101C depicts the cDNA sequence of human INTERCEPT 258 and the predicted amino acid sequence of INTERCEPT 258 (SEQ ID NO: 76). The open reading frame extends from nucleotide 153 to nucleotide 1262 of SEQ ID NO: 75.

FIG. 102 depicts a hydropathy plot of human INTERCEPT 258, the details of which are described herein. Below the hydropathy plot, the amino acid sequence of human INTERCEPT 258 is depicted.

FIG. 103A-103C depicts a cDNA sequence of mouse INTERCEPT 258 and the predicted amino acid sequence of mouse INTERCEPT 258 (SEQ ID NO: 78). The open reading frame extends from nucleotide 107 TO 1288 of SEQ ID NO: 77.

FIG. 104 depicts a hydropathy plot of mouse INTERCEPT 258, the details of which are described herein. Below the hydropathy plot, the amino acid sequence of mouse INTERCEPT 258 is depicted.

FIG. 105 depicts an alignment of the amino acid sequence of human INTERCEPT 258 and the amino acid sequence of mouse INTERCEPT 258. The alignment demonstrates that the amino acid sequences of human and mouse INTERCEPT 258 are 62.8% identical. This alignment was performed using the ALIGN program with a PAM120 scoring matrix, a gap length penalty of 12 and a gap penalty of 4.

FIG. 106 depicts an alignment of the amino acid sequence of human A33 antigen (Swiss Prot Accession Number Q99795; SEQ ID NO: 174) and the amino acid sequence of human INTERCEPT 258. The A33 antigen is a transmembrane glycoprotein and member of the Ig superfamily that may be a cancer cell marker. The amino acid sequences of A33 antigen and human INTERCEPT 258 are 23% identical. This alignment was performed using the ALIGN alignment program with a PAM 120 scoring matrix, a gap length penalty of 12, and a gap penalty of 4.

FIG. 107A-107F depicts an alignment of the nucleotide sequence of human A33 antigen (Gen Bank Accession Number U79725; SEQ ID NO: 175) and the nucleotide sequence of human INTERCEPT 258. These two nucleotide sequences are 40.6% identical. The nucleotide sequence of the open reading frame of human A33 antigen and that of human INTERCEPT 258 are 44% identical. These alignments were performed using the ALIGN alignment program with a PAM120 scoring matrix, a gap length penalty of 12, and a gap penalty of 4.

FIG. 108 depicts an alignment of the amino acid sequence of human A33 antigen (Swiss Prot Accession Number Q99795; SEQ ID NO: 174) and the amino acid sequence of mouse INTERCEPT 258. These two amino acid sequences have an overall amino acid identity of 23%. This alignment was performed using the ALIGN alignment program with a PAM120 scoring matrix, a gap length penalty of 12, and a gap penalty of 4.

FIG. 109A-109I depicts an alignment of the nucleotide sequence of human A33 antigen (GenBank Accession Number U79725; SEQ ID NO: 175) and the nucleotide sequence of mouse INTERCEPT 258. These two nucleotide sequences are 40% identical. The nucleotide sequence of the open reading frame of human A33 antigen and that of mouse INTERCEPT 258 are 43.2% identical. These alignments were performed using the ALIGN alignment program with a PAM120 scoring matrix, a gap length penalty of 12, and a gap penalty of 4.

FIG. 110A-110E depicts an alignment of the nucleotide sequence of human PECAM-1, (SEQ ID NO: 176) an integrin expressed on endothelial cells and the nucleotide sequence of human INTERCEPT 258. These two nucleotide sequences are 40.5% identical. This alignment was performed using ALIGN alignment program with a PAM120 scoring matrix, a gap length of 12, and a gap penalty of 4.

FIG. 111A-111D depicts the cDNA sequence and the predicted amino acid sequence of human TANGO 204 (SEQ ID NO: 80). The open reading frame extends from nucleotide 99 to nucleotide 890 of SEQ ID NO: 79.

FIG. 112 depicts a hydropathy plot of human TANGO 204, the details of which are described herein.

FIG. 113 depicts an alignment of the somatomedin B domain of human TANGO 204 with a consensus somatomedin B domain. In the consensus sequence, more conserved residues are indicated by uppercase letters, and less conserved residues are indicated by lowercase letters.

FIG. 114 depicts an alignment of the thrombospondin type 1 domain of human TANGO 204 with a consensus thrombospondin type 1 domain. In the consensus sequence, more conserved residues are indicated by uppercase letters, and less conserved residues are indicated by lowercase letters.

FIG. 115A-115B depicts the cDNA sequence and the predicted amino acid sequence of mouse TANGO 204 (SEQ ID NO: 82). The open reading frame extends from nucleotides 81 to 872 of SEQ ID NO: 81.

FIG. 116A-116C depicts an alignment of the open reading frames of human TANGO 204 and mouse TANGO 204.

FIG. 117 depicts an alignment of the amino acid sequences of human TANGO 204 and mouse TANGO 204.

FIG. 118A-118C depicts the cDNA sequence and the predicted amino acid sequence of human TANGO 206 (SEQ ID NO: 84). The open reading frame extends from nucleotide 99 to nucleotide 1358 of SEQ ID NO: 83.

FIG. 119 depicts a hydropathy plot of human TANGO 206, the details of which are described herein.

FIG. 120 depicts an alignment of the laminin EGF-like domain of human TANGO 206 with a consensus laminin EGF-like domain. In the consensus sequence, more conserved residues are indicated by uppercase letters, and less conserved residues are indicated by lowercase letters.

FIG. 121A-121D depicts the cDNA sequence and the predicted amino acid sequence of mouse TANGO 206 (SEQ ID NO: 86). The open reading frame extends from nucleotide 332-1591 (SEQ ID NO: 85).

FIG. 122A-122D depicts an alignment of the open reading frames of human TANGO 206 and mouse TANGO 206.

FIG. 123A-123B depicts an alignment of the amino acid sequences of human TANGO 206 and mouse TANGO 206.

FIG. 124A-124E depicts the cDNA sequence and the predicted amino acid sequence of human TANGO 209 (SEQ ID NO: 88). The open reading frame extends from nucleotide 194-1531 of SEQ ID NO: 87.

FIG. 125 depicts a hydropathy plot of human TANGO 209, the details of which are described herein.

FIG. 126 depicts an alignment of the thyroglobulin type 1 domains of human TANGO 209 with a consensus thyroglobulin type 1 domain. In the consensus sequence, more conserved residues are indicated by uppercase letters, and less conserved residues are indicated by lowercase letters.

FIG. 127 depicts an alignment of the Kazal-type serine protease inhibitor domains of human TANGO 209 with a consensus Kazal-type serine protease inhibitor domain. In the consensus sequence, more conserved residues are indicated by uppercase letters, and less conserved residues are indicated by lowercase letters.

FIG. 128A-128E depicts the cDNA sequence and the predicted amino acid sequence of mouse TANGO 209 (SEQ ID NO: 90). The open reading frame extends from nucleotide 187 to nucleotide 1527 of SEQ ID NO: 89.

FIG. 129A-129D depicts an alignment of the open reading frames of human TANGO 209 and mouse TANGO 209.

FIG. 130A-130B depicts an alignment of the amino acid sequences of human TANGO 209 and mouse TANGO 209.

FIG. 131 depicts the cDNA sequence and the predicted amino acid sequence of human TANGO 244 (SEQ ID NO: 92). The open reading frame extends from nucleotide 85 to nucleotide 570 of SEQ ID NO: 91.

FIG. 132 depicts a hydropathy plot of human TANGO 244, the details of which are described herein.

FIG. 133 depicts an alignment of the immunoglobulin domain of human TANGO 244 with a consensus hidden Markov model immunoglobulin domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 134 depicts an alignment of the amino acid sequence of human TANGO 244 and the amino acid sequence of human CTH (Genbank Accession Number AF061022; SEQ ID NO: 177; Marcuz et al., Eur J. Immunol. 28:4094-4104). This alignment was created using ALIGN (version 2.0; PAM120 scoring matrix; gap length penalty of 12; gap penalty of 4). In this alignment, the sequences are 48.6% identical.

FIG. 135A-135B depicts the cDNA sequence and the predicted amino acid sequence of human TANGO 246 (SEQ ID NO: 94). The open reading frame extends from nucleotide 94 to nucleotide 1080 of SEQ ID NO: 93.

FIG. 136 depicts a hydropathy plot of human TANGO 246, the details of which are described herein.

FIG. 137 depicts an alignment of the cell cycle protein domain of human TANGO 246 with a consensus hidden Markov model cell cycle protein domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 138 depicts an alignment of the ABC transporter domain of human TANGO 246 with a consensus hidden Markov model ABC transporter domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 139A-139D depicts the cDNA sequence and the predicted amino acid sequence of human TANGO 275 (SEQ ID NO: 96). The open reading frame extends from nucleotide 65 to nucleotide 3931 of SEQ ID NO: 95.

FIG. 140 depicts a hydropathy plot of human TANGO 275, the details of which are described herein.

FIG. 141A-141B depicts alignments of the EGF-like domains of human TANGO 275 with a consensus hidden Markov model EGF-like domain. The TANGO 275 EGF-like domains are at amino acids 99 to 126, 345 to 380, 564 to 600, 606 to 644, 650 to 687, 693 to 728, 734 to 769, 775 to 810, 816 to 850, 856 to 893, 983 to 1020, 1026 to 1061, 1072 to 1107, 1203 to 1238, and 1244 to 1283 of SEQ ID NO:96. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 142 depicts alignments of the TB domains of human TANGO 275 with a consensus hidden Markov model TB domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 143 depicts alignments of the metallothionein domain of human TANGO 275 (amino acids 694 to 708 of SEQ ID NO:96) with a consensus hidden Markov model metallothionein domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 144A-144H depicts an alignment of the nucleotide sequence of human TANGO 275 and the nucleotide sequence of mouse LTBP-3 (Genbank Accession Number L40459; SEQ ID NO: 178). This alignment was created using ALIGN (version 2.0; PAM 120 scoring matrix; gap length penalty of 12; gap penalty of 4). In this alignment, the sequences are 77.1% identical.

FIG. 145A-145C depicts an alignment of the amino acid sequence of human TANGO 275 and the amino acid sequence of mouse LTBP-3 (GENSEQ Accession Number R79475; SEQ ID NO: 179). This alignment was created using ALIGN (version 2.0; PAM 120 scoring matrix, gap length penalty of 12; gap penalty of 4). In this alignment, the sequences are 82.8% identical.

FIG. 146A-146G depicts the cDNA sequence and the predicted amino acid sequence of mouse TANGO 275 (SEQ ID NO: 98). The open reading frame extends from nucleotide 157 to nucleotide 3915 of SEQ ID NO: 97.

FIG. 147A-147B depicts the cDNA sequence and the predicted amino acid sequence of human MANGO 245 (SEQ ID NO: 100). The open reading frame extends from nucleotide 105 to nucleotide 1148 of SEQ ID NO: 99.

FIG. 148 depicts a hydropathy plot of human MANGO 245, the details of which are described herein.

FIG. 149A-149B depicts the cDNA sequence and the predicted amino acid sequence of monkey MANGO 245 (SEQ ID NO: 102). The open reading frame extends from nucleotide 250 to nucleotide 1236 of SEQ ID NO: 101.

FIG. 150 depicts an alignment of the amino acid sequences of human MANGO 245 and monkey MANGO 245. This alignment was created using ALIGN (version 2.0; PAM120 scoring matrix, gap length penalty of 12; gap penalty of 4). In this alignment, the sequences are 84.8% identical.

FIG. 151 depicts alignments of the CIq domains of human MANGO 245 with a consensus hidden Markov model CIq domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 152 depicts alignments of the CIq domains of monkey MANGO 245 with a consensus hidden Markov model CIq domain. In the consensus sequence, more conserved residues are indicated by uppercase letters, and less conserved residues are indicated by lowercase letters. A “−” within a sequence indicates a gap created in the sequence for purposes of alignment. A “+” between the aligned sequences indicates a conservative amino acid difference.

FIG. 153 depicts the cDNA sequence of mouse MANGO 245 and the predicted amino acid sequence of mouse MANGO 245 (SEQ ID NO: 104). The open reading frame extends from nucleotide 29 to nucleotide 625 of SEQ ID NO: 103.

FIG. 154A-154B depicts an alignment of nucleotide 51 to nucleotide 748 of human MANGO 245 with mouse MANGO 245. This alignment was created using BESTFIT (BLOSUM 62 scoring matrix; gap open penalty of 12; frame shift penalty of 5; gap extend penalty of 4). In this alignment, the sequences are 89.6% identical.

FIG. 155 depicts an alignment of the amino acid sequence of human TANGO 246 and the amino acid sequence of Arabidopsis thaliana AIG1 (Genbank Accession Number AAC49289; SEQ ID NO: 180).

FIG. 156A-156B depicts an alignment of the amino acid sequence of mouse TANGO 275 and the amino acid sequence of mouse LTBP-3 (GENSEQ Accession Number R79475; SEQ ID NO: 179). This alignment was created using ALIGN (version 2.0; PAM 120 scoring matrix, gap length penalty of 12; gap penalty of 4). In this alignment, the sequences are 97.4% identical.

FIG. 157A-157C depicts the cDNA sequence of human INTERCEPT 340 and the predicted amino acid sequence of INTERCEPT 340 (SEQ ID NO: 106). The open reading frame extends from nucleotide 1222 to nucleotide 1944 of SEQ ID NO: 105.

FIG. 158 depicts a hydropathy plot of human INTERCEPT 340, the details of which are described herein. Below the hydropathy plot, the numbers corresponding to the amino acid sequence of INTERCEPT 340 are indicated. The amino acid sequence of each of the fibrillar collagen C-terminal domains are indicated by underlining and the abbreviation “COLFI”.

FIG. 159 depicts an alignment of each of the fibrillar collagen C-terminal domains (also referred to herein as “COLF domains”) of human INTERCEPT 340 with consensus hidden Markov model COLF domains. For each alignment, the upper sequence is the consensus amino acid sequence, while the lower sequence amino acid sequence corresponds to amino acid 58 to amino acid 116, amino acid 126 to amino acid 151, and amino acid 186 to amino acid 217.

FIG. 160A-160C depicts the cDNA sequence of human MANGO 003 and the predicted amino acid sequence of MANGO 003 (SEQ ID NO: 108). The open reading frame extends from nucleotide 57 to nucleotide 1568 of SEQ ID NO: 107.

FIG. 161 depicts a hydropathy plot of human MANGO 003, the details of which are described herein. Below the hydropathy plot, the numbers corresponding to the amino acid sequence of MANGO 003 are indicated. The amino acid sequence of each of the immunoglobulin domains, and the neurotransmitter gated ion channel domain are indicated by underlining and the abbreviations “ig” and “neur chan”, respectively.

FIG. 162 depicts an alignment of each of the immunoglobulin domains (also referred to herein as “Ig domains”) of human MANGO 003 with the consensus hidden Markov model immunoglobulin domains. For each alignment, the upper sequence is the consensus sequence, while the lower sequence corresponds to amino acid 44 to amino acid 101, amino acid 165 to amino acid 223, and amino acid 261 to amino acid 340.

FIG. 163 depicts an alignment of the neurotransmitter gated ion channel domain of human MANGO 003 with the consensus hidden Markov model neurotransmitter gated ion channel domain. The upper sequence is the consensus sequence, while the lower sequence corresponds to amino acid 388 amino acid 397.

FIG. 164A-164B depicts the cDNA sequence of mouse MANGO 003 and the predicted amino acid sequence of MANGO 003 (SEQ ID NO: 110). The open reading frame extends from nucleotide 1 to nucleotide 626 of SEQ ID NO: 109.

FIG. 165 depicts a hydropathy plot of mouse MANGO 003, the details of which are described herein. Below the hydropathy plot, the numbers corresponding to the amino acid sequence of mouse MANGO 003 are indicated.

FIG. 166A-166B depicts the cDNA sequence of human MANGO 347 and the predicted amino acid sequence of MANGO 347 (SEQ ID NO: 112). The open reading frame extends from nucleotide 31 to nucleotide 444 of SEQ ID NO: 111.

FIG. 167 depicts a hydropathy plot of human MANGO 347, the details of which are described herein. Below the hydropathy plot, the numbers corresponding to the amino acid sequence of MANGO 347 are indicated. The amino acid sequence of the CUB domain is indicated by underlining and the abbreviation “CUB”.

FIG. 168 depicts an alignment of the CUB domain of human MANGO 347 with a consensus hidden Markov model CUB domain. The upper sequence is the consensus amino acid sequence, while the lower sequence corresponds to amino acid 40 to amino acid 136.

FIG. 169A-169F depicts the cDNA sequence of human TANGO 272 and the predicted amino acid sequence of TANGO 272 (SEQ ID NO: 114). The open reading frame extends from nucleotide 230 to nucleotide 3379 of SEQ ID NO: 113.

FIG. 170 depicts a hydropathy plot of human TANGO 272, the details of which are described herein. Below the hydropathy plot, the numbers corresponding to the amino acid sequence of TANGO 272 are indicated. The amino acid sequence of each of the fourteen EGF-like domains and the delta serrate ligand domain is indicated by underlining and the abbreviation “EGF-like” and “DSL”, respectively.

FIG. 171A-171D depicts an alignment of each of the EGF-like domains of human TANGO 272 with consensus hidden Markov model EGF-like domains. The upper sequence is the consensus amino acid sequence, while the lower sequence corresponds to amino acid 151 to amino acid 181; 200 to 229; 242 to 272; 285 to 315; 328 to 358; 378 to 5404; 417 to 447; 460 to 490; 503 to 533; 546 to 576; 589 to 619; 632 to 661; 674 to 704; and 717 to 747. For alignment of the delta serrate ligand domain, the upper sequence is the consensus hidden Markov model, while the lower sequence corresponds to amino acid 518 to amino acid 576.

FIG. 172A-172C depicts the cDNA sequence of mouse TANGO 272 and the predicted amino acid sequence of TANGO 272 (SEQ ID NO: 116). The open reading frame extends from nucleotide 1 to nucleotide 1492 of SEQ ID NO: 115.

FIG. 173 depicts a hydropathy plot of mouse TANGO 272, the details of which are described herein. Below the hydropathy plot, the numbers corresponding to the amino acid sequence of mouse TANGO 272 are indicated.

FIG. 174A-174B depicts the cDNA sequence of human TANGO 295 and the predicted amino acid sequence of TANGO 295 (SEQ ID NO: 118). The open reading frame extends from nucleotide 217 to nucleotide 684 of SEQ ID NO: 117.

FIG. 175 depicts a hydropathy plot of human TANGO 295, the details of which are described herein. Below the hydropathy plot, the numbers corresponding to the amino acid sequence of human TANGO 295 are indicated. The amino acid sequence of the pancreatic ribonuclease domain is indicated by underlining and the abbreviation “RNase A”.

FIG. 176 depicts an alignment of the pancreatic ribonuclease domain of human TANGO 295 with a consensus hidden Markov model pancreatic ribonuclease domain. The upper sequence is the consensus amino acid sequence, while the lower sequence corresponds to amino acid 32 to amino acid 156.

FIG. 177A-177B depicts the cDNA sequence of human TANGO 354 and the predicted amino acid sequence of TANGO 354 (SEQ ID NO: 120). The open reading frame extends from nucleotide 62 to nucleotide 976 of SEQ ID NO: 119.

FIG. 178 depicts a hydropathy plot of human TANGO 354, the details of which are described herein. Below the hydropathy plot, the numbers corresponding to the amino acid sequence of human TANGO 354 are indicated. The amino acid sequence of the immunoglobulin domain is indicated by underlining and the abbreviation “ig”.

FIG. 179 depicts an alignment of the immunoglobulin domain of human TANGO 354 with a consensus hidden Markov model immunoglobulin domains. The upper sequence is the consensus amino acid sequence, while the lower sequence corresponds to amino acid 33 to amino acid 110.

FIG. 180A-180D depicts the cDNA sequence of human TANGO 378 and the predicted amino acid sequence of TANGO 378 (SEQ ID NO: 122). The open reading frame extends from nucleotide 42 to nucleotide 1625 of SEQ ID NO: 121.

FIG. 181 depicts a hydropathy plot of human TANGO 378, the details of which are described herein. Below the hydropathy plot, the numbers corresponding to the amino acid sequence of human TANGO 378 are indicated. The amino acid sequence of the seven transmembrane domain is indicated by underlining and the abbreviation “7tm”.

FIG. 182 depicts an alignment of the seven transmembrane receptor domain of human TANGO 378 with a consensus hidden Markov model of this domain. The upper sequence is the consensus amino acid sequence, while the lower sequence corresponds to amino acid 187 to amino acid 516 of TANGO 378 (SEQ ID NO: 122). In this alignment an uppercase letter between the two sequences indicates an exact match, and a “+” indicates a similarity.

FIG. 183A-183C depicts a global alignment between the nucleotide sequence of the open reading frame (ORF) of SEQ ID NO: 107, human MANGO 003, and the nucleotide sequence of the open reading frame of SEQ ID NO: 109, mouse MANGO 003. The upper sequence is the human MANGO 003 ORF nucleotide sequence, while the lower sequence is the mouse MANGO 003 ORF nucleotide sequence. These nucleotides sequences share a 31.1% identity. The global alignment was performed using the ALIGN program version 2.0u (Matrix file used: pam 120.mat, gap penalties of −12/−4 with a global alignment score of −1212; Myers and Miller, 1989, CABIOS 4:11-7).

FIG. 184A-184B depicts a local alignment between the nucleotide sequence of human MANGO 003 and the nucleotide sequence of mouse MANGO 003. The upper sequence is the human MANGO 003 nucleotide sequence, while the lower sequence is the mouse MANGO 003 nucleotide sequence. These nucleotides sequences share a 62.8% identity over nucleotide 970 to nucleotide 2080 of the human MANGO 003 sequence (nucleotide 10 to nucleotide 1070 of mouse MANGO 003). The local alignment was performed using the L-ALIGN program version 2.0u54 Jul. 1996 (Matrix file used: pam 120.mat, gap penalties of −12/−4 with a score of 3241; Huang and Miller, 1991, Adv. Appl. Math. 12:373-381).

FIG. 185 depicts a global alignment between the amino acid sequence of human MANGO 003. The upper sequence is the human MANGO 003 amino acid sequence, while the lower sequence is the mouse MANGO 003 amino acid sequence. These amino acid sequences share a 30.1% identity. The global alignment was performed using the ALIGN program version 2.0u (Matrix file used: pam 120.mat, gap penalties of −12/−4 with a global alignment score of −488; Myers and Miller, 1989, CABIOS 4:11-7).

FIG. 186A-186E depicts a global alignment between the nucleotide sequence of the open reading frame (ORF) of human TANGO 272 and the nucleotide sequence of the open reading frame of mouse TANGO 272. The upper sequence is the mouse TANGO 272 ORF nucleotide sequence, while the lower sequence is the human TANGO 272 ORF nucleotide sequence. These nucleotides sequences share a 39.1% identity. The global alignment was performed using the ALIGN program version 2.0u (matrix file used: pam 120.mat, gap penalties of −12/−4 with a global alignment score of −79; Myers and Miller, 1989, CABIOS 4:11-7).

FIG. 187A-187C depicts a local alignment between the nucleotide sequence of human TANGO 272 and the nucleotide sequence of mouse TANGO 272. The upper sequence is the human TANGO 272 nucleotide sequence, while the lower sequence is the mouse TANGO 272 nucleotide sequence. These nucleotides sequences share a 67.6% identity over nucleotide 1890 to nucleotide 4610 of the human TANGO 272 sequence (nucleotide 10 to nucleotide 2560 of mouse TANGO 272). The local alignment was performed using the L-ALIGN program version 2.0u54 Jul. 1996 (Matrix file used: pam 120.mat, gap penalties of −12/−4 with a score of 8462; Huang and Miller, 1991, Adv. Appl. Math. 12:373-381).

FIG. 188A-188B depicts a global alignment between the amino acid sequence of human TANGO 272 and the amino acid sequence of mouse TANGO 272. The upper sequence is the human TANGO 272 amino acid sequence, while the lower sequence is the mouse TANGO 272 amino acid sequence. These amino acid sequences share a 38.2% identity. The global alignment was performed using the ALIGN program version 2.0u (Matrix file used: pam 120.mat, gap penalties of −12/−4 with a global alignment score of −19; Myers and Miller, 1989, CABIOS 4:11-7).

FIG. 189A-198D depicts the cDNA sequence of rat TANGO 272 and the predicted amino acid sequence of TANGO 272 (SEQ ID NO: 124). The open reading frame extends from nucleotide 925 to nucleotide 2832 of SEQ ID NO: 123.

FIG. 190A-190H depicts a global alignment between the nucleotide sequence of human TANGO 272 and the nucleotide sequence of rat TANGO 272. The upper sequence is the human TANGO 272 nucleotide sequence, while the lower sequence is the rat TANGO 272 nucleotide sequence. These nucleotides sequences share a 55.7% identity. The global alignment was performed using the ALIGN program version 2.0u (Matrix file used: pam 120.mat, gap penalties of −12/−4 with a global alignment score of 8635; Myers and Miller, 1989, CABIOS 4:11-7).

FIG. 191A-191F depicts a global alignment between the nucleotide sequence of mouse TANGO 272 and the nucleotide sequence of rat TANGO 272. The upper sequence is the mouse TANGO 272 nucleotide sequence, while the lower sequence is the rat TANGO 272 nucleotide sequence. These nucleotides sequences share a 43.7% identity. The global alignment was performed using the ALIGN program version 2.0u (Matrix file used: pam 120.mat, gap penalties of −12/−4 with a global alignment score of 2827; Myers and Miller, 1989, CABIOS 4:11-7).

FIG. 192 depicts a global alignment of the human TANGO 295 and GenPept AF037081 amino acid sequences. The upper sequence is the human TANGO 295 sequence, while the lower sequence is the GenPept AF037081 (SEQ ID NO: 181) sequence. GenPept AF037081 encodes a ribonuclease k6 protein. The global alignment revealed a 53.2% identity between these two sequences (Matrix file used: pam 120.mat, gap penalties of −12/−4 with a global alignment score of 405; Myers and Miller, 1989, CABIOS 4:11-7).

FIG. 193A-193C depicts a global alignment of the human TANGO 295 and GenPept AF037081 nucleotide sequences. The upper sequence is the human TANGO 295 sequence, while the lower sequence is the GenPept AF037081 (SEQ ID NO: 181) sequence. The global alignment revealed a 22.6% identity between these two sequences (Matrix file used: pam 120.mat, gap penalties of −12/−4 with a global alignment score of −2718; Myers and Miller, 1989, CABIOS 4:11-7).

FIG. 194A-194B depicts a local alignment of the human TANGO 295 and GenPept AF037081 nucleotide sequences. The upper sequence is the human TANGO 295 sequence, while the lower sequence is the GenPept AF037081 (SEQ ID NO: 181) sequence. The local alignment revealed a 62.7% identity between nucleotide 235 to nucleotide 687 of human TANGO 295, and nucleotide 3 to nucleotide 453 of AF037081 (SEQ ID NO: 181); 43.4% identity between nucleotide 410 to nucleotide 850 of human TANGO 295, and nucleotide 3 to nucleotide 450 of AF037081 (SEQ ID NO: 181); and 46.5% identity between nucleotide 432 to nucleotide 700 of human TANGO 295, and nucleotide 5 to nucleotide 251 of AF037081 (Matrix file used: pam 120.mat, gap penalties of −12/−4 with a global alignment score of 1214; Huang and Miller, 1991, Adv. Appl. Math. 12:373-381).

FIG. 195A-195B depicts an alignment of each of the EGF-like domains and laminin-EGF-like domains of mouse TANGO 272 with consensus hidden Markov model EGF-like domains. For alignments of the EGF-like domains, the upper sequence is the consensus amino acid sequence, while the lower sequence corresponds to amino acids 37-67; amino acid 80 to amino acid 110; amino acid 123 to amino acid 153; and amino acid 166 to amino acid 196. For alignments of the laminin/EGF-like domains, the upper sequence is the consensus hidden Markov model domain, while the lower sequence corresponds to amino acid 3 to amino acid 37; amino acid 41 to amino acid 80; amino acid 83 to amino acid 123; and amino acid 127 to amino acid 172. For alignment of the delta serrate ligand(DSL) domain, the upper sequence is the consensus hidden Markov model domain, while the lower sequence corresponds to amino acid 10 to amino acid 67.

FIG. 196 depicts a hydropathy plot of rat TANGO 272, the details of which are described herein. Below the hydropathy plot, the numbers corresponding to the amino acid sequence of rat TANGO 272 are indicated.

FIG. 197A-197D depicts an alignment of each of the EGF-like domains and laminin-EGF-like domains of rat TANGO 272 with consensus hidden Markov model of EGF-like domains. For alignments of the EGF-like domains, the upper sequence is the consensus amino acid sequence, while the lower sequence corresponds to amino acid 18 to amino acid 48; 61 to 91; 105-137; 150-180; 193-223; 236-266; 279-309; 322-352; 365-394; 407-437; and 450-480. For alignments of the laminin/EGF-like domains, the upper sequence is the consensus hidden Markov model domain, while the lower sequence corresponds to 22-61; 65-105; 109-150; 154-193; 197-236; 240-279; 283-322; 326-365; 368-407; 411-450; and 454-489. For alignment of the delta serrate-ligand domain, the upper sequence is the consensus hidden Markov model domain, while the lower sequence corresponds to amino acids 246-309.

FIG. 198A-198B depicts the cDNA sequence of human TANGO 339 and the predicted amino acid sequence of human TANGO 339 (SEQ ID NO: 126). The open reading frame extends from nucleotide 210 to nucleotide 1019 of SEQ ID NO: 125.

FIG. 199 depicts a hydropathy plot of human TANGO 339, the details of which are described herein. The dashed vertical line separates the signal sequence (amino acids 1 to 42) on the left from the mature protein (amino acids 43 to 270) on the right.

FIG. 200 depicts an alignment of the amino acid sequence of human CD9 antigen (Accession Number NM_{—}001769 of SEQ ID NO: 125) and the amino acid sequence of human TANGO 339. The amino acid sequences of human CD9 antigen and human TANGO 339 are 24.1% identical. This alignment was performed using the ALIGN alignment program with a PAM120 scoring matrix, a gap length penalty of 12, and a gap penalty of 4.

FIG. 201A-201B depicts an alignment of the nucleotide sequence of the coding region of human CD9 antigen (Accession Number NM_{—}001769; SEQ ID NO: 182) and the nucleotide sequence of the coding region of human TANGO 339. The nucleotide sequences of the coding regions of human CD9 antigen and human TANGO 339 are 45.9% identical. This alignment was performed using the ALIGN alignment program with a PAM120 scoring matrix, a gap length penalty of 12, and a gap penalty of 4.

FIG. 202 depicts a cDNA sequence of human TANGO 358 and the predicted amino acid sequence of human TANGO 358 (SEQ ID NO: 128). The open reading frame of human TANGO 358 extends from nucleotide 184 to 429 of SEQ ID NO: 127.

FIG. 203 depicts a hydropathy plot of human TANGO 358, the details of which are described herein.

FIG. 204 depicts the cDNA sequence of human TANGO 365 and the predicted amino acid sequence of human TANGO 365 (SEQ ID NO: 130). The open reading frame extends from nucleotide 56 to nucleotide 550 of SEQ ID NO: 129.

FIG. 205 depicts a hydropathy plot of human TANGO 365, the details of which are described herein.

FIG. 206 depicts the cDNA sequence of human TANGO 368 and the predicted amino acid sequence of TANGO 368 (SEQ ID NO: 132). The open reading frame of human TANGO 368 extends from nucleotide 152 to nucleotide 328 of SEQ ID NO: 131.

FIG. 207 depicts a hydropathy plot of human TANGO 368, the details of which are described herein.

FIG. 208A-208B depicts a local alignment of the nucleotide sequence of full-length human TANGO 368 and a fragment of the human T-cell receptor gamma V1 gene region (Accession Number AF057177; SEQ ID NO: 183). The nucleotide sequence of human TANGO 368 and the human T-cell receptor gamma V1 gene region are 99.3% identical for a 973 bp overlap. This alignment was performed using the LALIGN program with a PAM120 scoring matrix, a gap length penalty of 12 and a gap penalty of 4.

FIG. 209 depicts a cDNA sequence of human TANGO 369 and the predicted amino acid sequence of human TANGO 369 (SEQ ID NO: 134). The open reading frame of human TANGO 369 extends from nucleotide 162 to 335 of SEQ ID NO: 133.

FIG. 210 depicts a hydropathy plot of human TANGO 369, the details of which are described herein.

FIG. 211 depicts the cDNA sequence of human TANGO 383 and the predicted amino acid sequence of human TANGO 383 (SEQ ID NO: 136). The open reading frame of human TANGO 383 extends from nucleotide 104 to nucleotide 523 of SEQ ID NO: 135.

FIG. 212 depicts a hydropathy plot of human TANGO 383, the details of which are described herein.

FIG. 213 depicts an alignment of the amino acid sequence of TANGO 383 and the amino acid sequence of Neuronal Thread Protein AD7C-NTP. The alignments demonstrates that the amino acid sequences of TANGO 383 and Neuronal Thread Protein AD7C-NTP (SEQ ID NO: 184) are 52% identical. This alignment was performed using the ProDom NCBI-BLASTP2 program with graphical output using the following settings: Matrix: BLOSUM62; Expect: 0.1; Filter: none.

FIG. 214 depicts the cDNA sequence of human MANGO 346 and the predicted amino acid sequence of human MANGO 346 (SEQ ID NO: 138). The open reading frame extends from nucleotide 319 to nucleotide 498 of SEQ ID NO: 137.

FIG. 215 depicts a hydropathy plot of human MANGO 346, the details of which are described herein.

FIG. 216A-216B depicts the cDNA sequence of human MANGO 349 and the predicted amino acid sequence of human MANGO 349 (SEQ ID NO: 140). The open reading frame of human MANGO 349 extends from nucleotide 221 to nucleotide 721 of SEQ ID NO: 139.

FIG. 217 depicts a hydropathy plot of human MANGO 349, the details of which are described herein.

FIG. 218A-218B depicts the cDNA sequence of INTERCEPT 307 and the predicted amino acid sequence of human INTERCEPT 307. The open reading frame of INTERCEPT 307 extends from nucleotides 45 to 1130.

FIG. 219 depicts a hydropathy plot of human INTERCEPT 307, the details of which are described herein.

FIG. 220 depicts an alignment of the amino acid sequence of PB39; Accession Number NM_{—}003627; SEQ ID NO: 185 and the amino acid sequence of human INTERCEPT 307. The amino acid sequences of human PB39 and human INTERCEPT 307 are 21.0% identical. This alignment was performed using the ALIGN alignment program with a PAM120 scoring matrix, a gap length penalty of 12, and a gap penalty of 4.

FIG. 221A-221C depicts an alignment of the nucleotide sequence of the coding region of PB39; Accession Number AF045584 (SEQ ID NO: 186) and the nucleotide sequence of the coding region of human INTERCEPT 307. The nucleotide sequences of the coding regions of PB39 and human INTERCEPT 307 are 40.9% identical. The full-length nucleic acid sequences of PB39 (Accession Number NM_{—}003627; SEQ ID NO: 185 and human INTERCEPT 307 are 44.0% identical. These alignments were performed using the ALIGN alignment program with a PAM120 scoring matrix, a gap length penalty of 12, and a gap penalty of 4.

FIG. 222 depicts an alignment of the human INTERCEPT 307 amino acid sequence with the human eosinophil granule major basic protein amino acid sequence (Accession Number Z26248; SEQ ID NO: 187). The amino acid sequences of INTERCEPT 307 and human eosinophil granule major basic protein are 13.8% identical. This alignment was performed using the ALIGN alignment program with a PAM120 scoring matrix, a gap length penalty of 12, and a gap penalty of 4.

FIG. 223A-223B shows an alignment of the nucleotide sequence of INTERCEPT 307 coding region and the nucleotide sequence of human eosinophil granule major basic protein coding region (Accession Number Z26248; SEQ ID NO: 187). The nucleotide sequences of the coding regions are 38.1% identical. The full-length INTERCEPT 307 nucleic acid sequence and human eosinophil granule major basic protein cDNA (Accession Number Z26248) have an overall sequence identity of 57.3%. These alignments were performed using the ALIGN alignment program with a PAM120 scoring matrix, a gap length penalty of 12, and a gap penalty of 4.

FIG. 224A-224B depicts the cDNA sequence of human MANGO 511 and the predicted amino acid sequence of human MANGO 511 (SEQ ID NO: 144). The open reading frame of human MANGO 511 extends from nucleotide 108 to 1004 of SEQ ID NO: 143.

FIG. 225 depicts a hydropathy plot of human MANGO 511, the details of which are described herein.

FIG. 226 depicts a local alignment of the amino acid sequence of leukocyte Ig-like receptor-1 (LIR-1; Accession Number AAB63522) and the amino acid sequence of human MANGO 511. The amino acid sequences of human LIR-1 and human MANGO 511 are 59.2% identical over the 233 amino acid overlap region that was analyzed. This alignment were performed using the LALIGN version 2.0, July 1996, local alignment program with a PAM120 scoring matrix, a gap length penalty of 12, and a gap penalty of 4. A global alignment of the amino acid sequence of leukocyte Ig-like receptor-1 (LIR-1; Accession Number AAB63522; SEQ ID NO: 188) and the amino acid sequence of human MANGO 511 reveals that the amino acid sequences of human LIR-1 and human MANGO 511 are 24.2% identical. This alignment was performed using the ALIGN alignment program with a PAM120 scoring matrix, a gap length penalty of 12, and a gap penalty of 4.

FIG. 227A-227C depicts an alignment of the nucleotide sequence of the coding region of LIR-1 (Accession Number AF009221; SEQ ID NO: 189) and the nucleotide sequence of the coding region of human MANGO 511. The nucleotide sequences of the coding regions of LIR-1 and human MANGO 511 are 34.0% identical. The full-length nucleic acid sequence of MANGO 511 and the coding region of LIR-1 are 44.0% identical. These alignments were performed using the ALIGN alignment program with a PAM120 scoring matrix, a gap length penalty of 12, and a gap penalty of 4.

FIG. 228A-228C depicts the cDNA sequence of TANGO 361 and the predicted amino acid sequence of TANGO 361 (SEQ ID NO: 146). The open reading frame of TANGO 361 extends from nucleotides 41 to 1309 of SEQ ID NO: 145.

FIG. 229 depicts a hydropathy plot of TANGO 361, the details of which are described herein.

FIG. 230 depicts the cDNA sequence of TANGO 499 form 1, variant 1 and the predicted amino acid sequence of TANGO 499 form 1, variant 1 (SEQ ID NO: 148). The open reading frame of TANGO 499 form 1, variant 1 extends from nucleotides 83 to 844 of SEQ ID NO: 147.

FIG. 231 depicts a hydropathy plot of TANGO 499 form 1, variant 1, the details of which are described herein.

FIG. 232 shows an alignment of the human TANGO 499 form 1, variant 1 amino acid sequence with the artemin amino acid sequence. The alignment shows that there is a 23.5% overall amino acid sequence identity between TANGO 499 form 1, variant 1 and Artemin. This alignment was performed using the ALIGN alignment program with a PAM120 scoring matrix, a gap length penalty of 12, and a gap penalty of 4.

FIG. 233 shows an alignment of the human TANGO 499 form 1, variant 1 amino acid sequence with the riboflavin binding protein amino acid sequence. The alignment shows that there is a 19.9% overall amino acid sequence identity between TANGO 499 form 1, variant 1 and riboflavin binding protein (SEQ ID NO: 190). This alignment was performed using the ALIGN alignment program with a PAM120 scoring matrix, a gap length penalty of 12, and a gap penalty of 4.

FIG. 234 depicts the cDNA sequence of TANGO 499 form 2, variant 3 and the predicted amino acid sequence of TANGO 499 form 2, variant 3 (SEQ ID NO: 150). The open reading frame of TANGO 499 form 2, variant 3 extends from nucleotides 144 to 830 of SEQ ID NO: 149.

FIG. 235 depicts a hydropathy plot of TANGO 499 form 2, variant 3, the details of which are described herein.

FIG. 236 shows an alignment of the TANGO 499 form 1, variant 1 amino acid sequence with the TANGO 499 form 2, variant 3 amino acid sequence. The alignment shows an alternative spliced exon which is present in form 1 and absent in form 2 and that there is a 90.2% overall amino acid sequence identity between human TANGO 499 form 1, variant 1 and the TANGO 499 form 2, variant 3. This alignment was performed using the ALIGN alignment program with a PAM120 scoring matrix, a gap length penalty of 12, and a gap penalty of 4.

FIG. 237 depicts a cDNA sequence of human TANGO 315 form 1 and the predicted human TANGO 315 form 1 amino acid sequence encoded by the sequence (SEQ ID NO: 152). The open reading frame of TANGO 315, form 1, comprises nucleotide 1 to nucleotide 888 of SEQ ID NO: 151.

FIG. 238 depicts a hydropathy plot of human TANGO 315 form 1, the details of which are described herein.

FIG. 239 depicts an alignment of the amino acid of the human TANGO 315 form 1 and the amino acid sequence of CD33 (NP_{—}001763; SEQ ID NO: 191). The alignment shows that there is a 59.4% overall amino acid sequence identity between TANGO 315 form 1 sequence and CD33. This alignment was performed using the ALIGN alignment program with a PAM120 scoring matrix, a gap length penalty of 12, and a gap penalty of 4.

FIG. 240A-240B depicts an alignment of the nucleotide sequence of the coding region of CD33 (NM_{—}001772; SEQ ID NO: 192) and the nucleotide sequence of the coding region of human TANGO 315 form 1. The nucleotide sequences of the coding regions of CD33 and human TANGO 315 form 1 are 75.8% identical. The nucleic acid sequence of CD33 (NM_{—}001772) and the human TANGO 315 form 1 nucleic acid sequence are 67.7% identical. These alignments were performed using the ALIGN alignment program with a PAM120 scoring matrix, a gap length penalty of 12, and a gap penalty of 4.

FIG. 241 depicts an alignment of the amino acid of TANGO 315 form 1 and the amino acid sequence of OB-BP-1 (Accession Number AAB70702; SEQ ID NO: 193). The alignment shows that there is a 52.8% overall amino acid sequence identity between the TANGO 315 form 1 sequence and Ob binding protein. This alignment was performed using the ALIGN alignment program with a PAM120 scoring matrix, a gap length penalty of 12, and a gap penalty of 4.

FIG. 242A-242B depicts an alignment of the nucleotide sequence of human TANGO 315 form 1 coding region and the nucleotide sequence of human OB-BP-1 coding region (Accession Number U71382; SEQ ID NO: 194). The nucleotide sequences of the coding regions are 74.2% identical. The nucleotide sequence of the TANGO 315 form 1 and the human OB-BP-1 cDNA (Accession Number U71382) have an overall sequence identity of 65%. These alignments were performed using the ALIGN alignment program with a PAM 120 scoring matrix, a gap length penalty of 12, and a gap penalty of 4.

FIG. 243A-243B depicts a cDNA sequence of human TANGO 315 form 2 and the predicted TANGO 315 form 2 amino acid sequence (SEQ ID NO: 154). The open reading frame of TANGO 315, form 2, comprises nucleotide 58 to nucleotide 888 of SEQ ID NO: 153.

FIG. 244 depicts a hydropathy plot of TANGO 315 form 2, the details of which are described herein.

FIG. 245 depicts an alignment of the amino acid of the TANGO 315 form 2 and the amino acid sequence of CD33 (NP_{—}001763; SEQ ID NO: 195). The alignment shows that there is a 62% overall amino acid sequence identity between the TANGO 315 form 2 sequence and CD33. This alignment was performed using the ALIGN alignment program with a PAM120 scoring matrix, a gap length penalty of 12, and a gap penalty of 4.

FIG. 246A-246B depicts a local alignment of the nucleotide sequence of CD33 (NM_{—}001772) and the nucleotide sequence of human TANGO 315 form 2. The nucleotide sequences of CD33 and human TANGO 315 form 2 are 75.4% identical. These alignments were performed using the ALIGN alignment program with a PAM120 scoring matrix, a gap length penalty of 12, and a gap penalty of 4.

FIG. 247 depicts an alignment of the amino acid of the TANGO 315 form 2 and the amino acid sequence of OB-BP-1 (Accession Number AAB70702; SEQ ID NO: 193). The alignment shows that there is a 53.3% overall amino acid sequence identity between the TANGO 315 form 2 sequence and Ob binding protein. This alignment was performed using the ALIGN alignment program with a PAM120 scoring matrix, a gap length penalty of 12, and a gap penalty of 4.

FIG. 248A-248B depicts an alignment of the nucleotide sequence of human TANGO 315 form 2 coding region and the nucleotide sequence of human OB-BP-1 coding region (Accession Number U71382; SEQ ID NO: 195). The nucleotide sequences of the coding regions are 73.2% identical. These alignments were performed using the ALIGN alignment program with a PAM120 scoring matrix, a gap length penalty of 12, and a gap penalty of 4.

FIG. 249A-249D depicts a cDNA sequence of TANGO 330 form 1 and the predicted human TANGO 330 form 1 amino acid sequence encoded by the sequence (SEQ ID NO: 156). The open reading frame of TANGO 330, form 1, comprises nucleotide 2 to nucleotide 2803 of SEQ ID NO: 155.

FIG. 250A-250C depicts a cDNA sequence of TANGO 330 form 2 and the predicted of human TANGO 330 form 2 amino acid sequence encoded by the sequence (SEQ ID NO: 158). The open reading frame of TANGO 330 form 2 comprises nucleotide 9 to nucleotide 1448 of SEQ ID NO: 157.

FIG. 251A-251G depicts a local alignment of the nucleotide sequence of human Roundabout; Accession Number AF040990; SEQ ID NO: 196) and the nucleotide sequence of the human TANGO 330 form 1. The nucleotide sequence of the human Roundabout and the human TANGO 330 form 1 nucleotide sequence are 56.9% identical.

FIG. 252A-252B depicts an alignment of the amino acid sequence of human Roundabout (Accession Number AAC39575; SEQ ID NO: 197) and the amino acid sequence of the human TANGO 330 form 1. The amino acid sequence of the human Roundabout and the human TANGO 330 form 1 are 26.6% identical. This alignment was performed using the ALIGN alignment program with a PAM120 scoring matrix, a gap length penalty of 12, and a gap penalty of 4.

FIG. 253A-253F depicts an alignment of the nucleotide sequence of the TANGO 330 form 1 and the nucleotide sequence of the human TANGO 330 form 2. The nucleotide sequences of TANGO 330 form 1 and TANGO 330 form 2 are 97.4% identical. These alignments were performed using the ALIGN alignment program with a PAM120 scoring matrix, a gap length penalty of 12, and a gap penalty of 4.

FIG. 254 depicts an alignment of the amino acid sequence of the TANGO 330 form 1 and the amino acid sequence of the TANGO 330 form 2. When the amino acid sequence of TANGO 330 form 2 is aligned with the amino acid sequence of TANGO 330 form 1, the fragments that are aligned are 94.1% identical. This alignment was performed using the ALIGN alignment program with a PAM120 scoring matrix, a gap length penalty of 12, and a gap penalty of 4.

FIG. 255A-255D depicts the nucleotide sequence of human TANGO 437 with the predicted amino acid sequence of human TANGO 437 (SEQ ID NO: 160). The open reading frame of human TANGO 437 extends from nucleotide 43 to nucleotide 1815 of SEQ ID NO: 159.

FIG. 256 depicts a hydropathy plot of human TANGO 437, the details of which are described herein.

FIG. 257A-257B depicts a local alignment of the nucleotide sequence of the coding region of human TANGO 437 with the nucleotide sequence of Gene 100 published in PCT Application No. WO98/39448 (V59610; SEQ ID NO: 198). Nucleic acids 101 to 798 of the nucleotide sequence of the coding region of human TANGO 437 and nucleic acids 1 to 573 of the nucleotide sequence of Gene 100 are 54.6% identical. Nucleic acids 1851 to 3679 of the full-length nucleotide sequence of TANGO 437 and nucleic acids 1 to 1751 of the nucleotide sequence of Gene 100 are 74.1% identical. These alignments were performed using the ALIGN alignment program with a PAM120 scoring matrix, a gap length penalty of 12, and a gap penalty of 4.

FIG. 258A-258B depicts the cDNA sequence of TANGO 480 and the predicted amino acid sequence of TANGO 480 (SEQ ID NO: 162). The open reading frame of TANGO 480 extends from nucleotide 43 to nucleotide 621 of SEQ ID NO: 161.

FIG. 259 depicts a hydropathy plot of TANGO 480, the details of which are described herein.

FIG. 260A-260E depicts the nucleotide sequence of human TANGO 437-form 2 with the predicted amino acid sequence of human TANGO 437-form 2 (SEQ ID NO: 164).

The open reading frame of human TANGO 437-form 2 extends from nucleotide 43 to nucleotide 2298 of SEQ ID NO: 163.

FIG. 261 depicts a hydropathy plot of human TANGO 437-form 2, the details of which are described herein.

### DETAILED DESCRIPTION OF THE INVENTION

The INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 proteins and nucleic acid molecules comprise families of molecules having certain conserved structural and functional features among family members. Examples of conserved structural domains include signal sequence (or signal peptide or secretion signal), transmembrane domains, cytoplasmic domains and extracellular domains.

As used herein, the terms “family” or “families” are intended to mean two or more proteins or nucleic acid molecules having a common structural domain and having sufficient amino acid or nucleotide sequence identity as defined herein. Family members can be from either the same or different species. For example, a family can comprise two or more proteins of human origin, or can comprise one or more proteins of human origin and one or more of non-human origin. Members of the same family may also have common structural domains.

As used herein, a “signal sequence” includes a peptide of at least about 15 or 20 amino acid residues in length which occurs at the N-terminus of secretory and membrane-bound proteins and which contains at least about 70% hydrophobic amino acid residues such as alanine, leucine, isoleucine, phenylalanine, proline, tyrosine, tryptophan, or valine. In a preferred embodiment, a signal sequence contains at least about 10 to 40 amino acid residues, preferably about 19-34 amino acid residues, and has at least about 60-80%, more preferably at least about 65-75%, and more preferably at least about 70% hydrophobic residues. A signal sequence serves to direct a protein containing such a sequence to a lipid bilayer. A signal sequence is usually cleaved during processing of the mature protein.

As used herein, a “transmembrane domain” refers to an amino acid sequence having at least about 25 to 40 amino acid residues in length and which contains hydrophobic amino acid residues such as alanine, leucine, isoleucine, phenylalanine, proline, tyrosine, tryptophan, or valine. In a preferred embodiment, a transmembrane domain contains at least about 25 to 40 amino acid residues, preferably about 25-30 amino acid residues, and has at least about 60-80% hydrophobic residues.

As used herein, a “cytoplasmic loop” includes an amino acid sequence located within a cell or within the cytoplasm of a cell and is typically associated with a transmembrane protein segment which extends through the cellular membrane to the extracellular region.

As used herein, an “extracellular domain” is a protein structural domain which is part of a transmembrane protein and resides outside the cell membrane, or is extracytoplasmic. A protein which has more than one transmembrane domain likewise has more than one extracellular domain. When located at the N-terminal domain the extracellular domain is referred to herein as an “N-terminal extracellular domain”. As used herein, an “N-terminal extracellular domain” includes an amino acid sequence. The N-terminal extracellular domain can be at least 10 amino acids in length or more, about 25, about 50, about 100, about 150, about 250, about 300, about 350, about 400, about 450, about 500, about 550, about 600, about 650, about 700, or more than about 750 amino acids.

The N-terminal extracellular domain is located outside of a cell or is extracellular. The C-terminal amino acid residue of a “N-terminal extracellular domain” is adjacent to an N-terminal amino acid residue of a transmembrane domain in a naturally-occurring protein. Preferably, the N-terminal extracellular domain is capable of interacting (e.g., binding to) with an extracellular signal, for example, a ligand (e.g., a glycoprotein hormone) or a cell surface receptor (e.g., an integrin receptor). Most preferably, the N-terminal extracellular domain mediates a variety of biological processes, for example, protein-protein interactions, signal transduction and/or cell adhesion.

### TANGO 136

The present invention is based in part on the discovery of cDNA molecules encoding mouse and human TANGO 136, a transmembrane protein.

A cDNA encoding a portion of mouse TANGO 136 was identified using a screening process which selects for nucleotide sequences which encode secreted proteins. A detailed description of this method, called “signal trapping” is provided in PCT Publication No. WO 98/22491, published May 28, 1998. In brief, a randomly primed cDNA library was prepared using cDNA prepared from mRNA extracted from lipopolysaccharide-stimulated mouse macrophages. To prepare this library, the cDNA was inserted into the mammalian expression vector pMEAP adjacent to a cDNA encoding placental alkaline phosphatase which lacks a secretory signal. Next, the cDNA library was amplified in bacteria. The amplified cDNA was then isolated and transfected into human 293T cells. After 28 hours, cell supernatants were collected and assayed for alkaline phosphatase activity. Clones giving rise to detectable alkaline phosphatase activity in the supernatant of transfected cells were isolated and analyzed further by sequencing and the novel clones subjected to further sequencing.

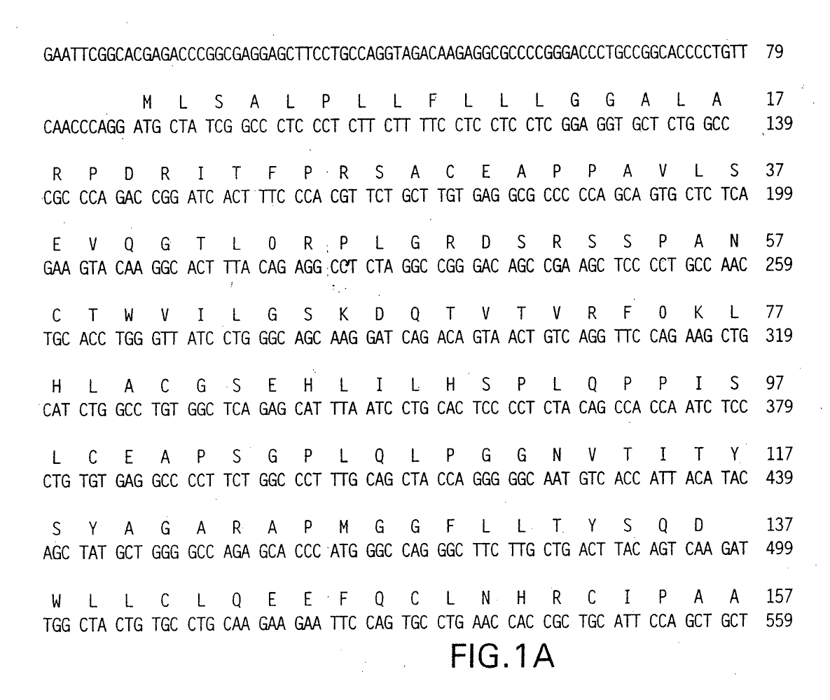

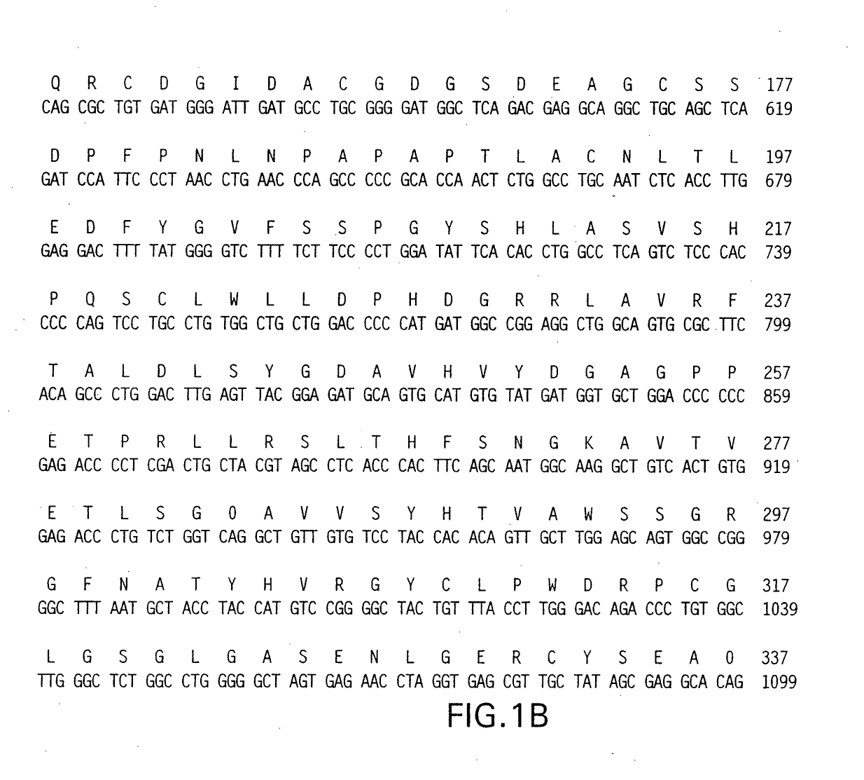

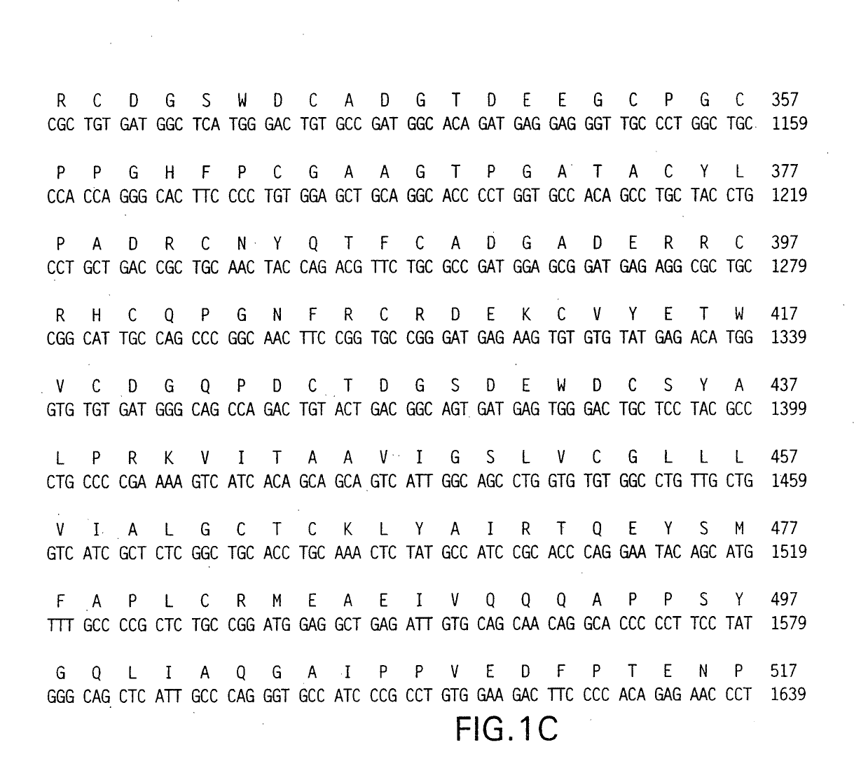

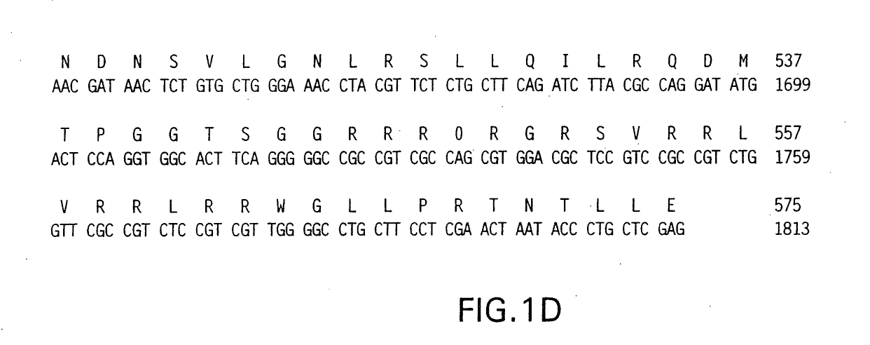

One such clone, mouse TANGO 136, was identified. This clone includes a 1813 nucleotide cDNA (FIG. 1A-1D; SEQ ID NO: 1). The open reading frame of this cDNA (nucleotides 89 to 1813 of SEQ ID NO: 1) encodes a 575 amino acid putative type I membrane protein (SEQ ID NO:2). Because no translation stop codon occurs at the end of the open reading frame, this cDNA is likely to be a partial cDNA which does not encode the most carboxy terminal portion of mouse TANGO 136.

The signal peptide prediction program SIGNALP (Nielsen et al. (1997) Protein Engineering 10:1-6) predicted that mouse TANGO 136 includes a 17 amino acid signal peptide (amino acid 1 to about amino acid 17 of SEQ ID NO:2) preceding the 558 amino acid (partial) mature protein (about amino acid 18 to amino acid 575 of SEQ ID NO:2). Mature mouse TANGO 136 has an extracellular domain (amino acids 18 to 441 of SEQ ID NO:2); a transmembrane domain (about amino acids 442 to 462 of SEQ ID NO:2); and a cytoplasmic domain (about amino acids 463 to 575 of SEQ ID NO:2).

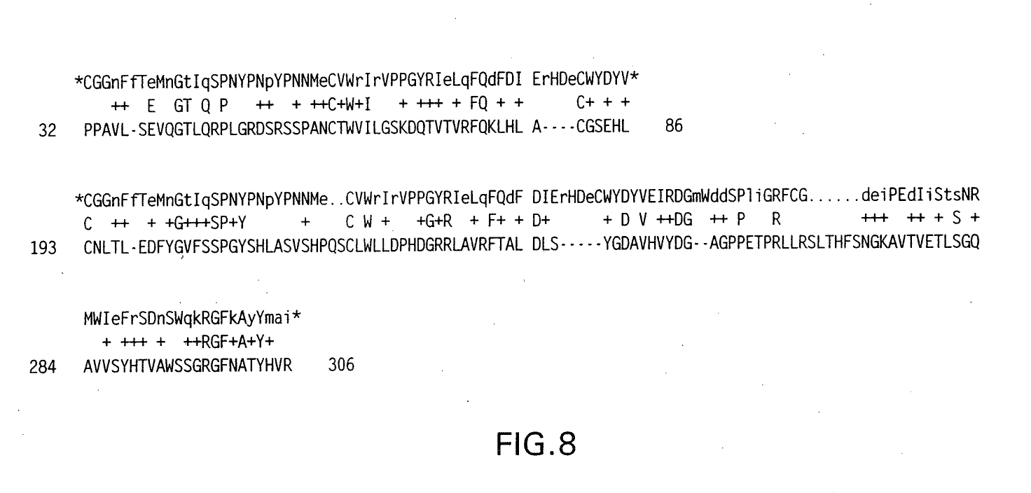

The extracellular region of mouse TANGO 136 includes two CUB-like domains (amino acids 32 to 86 and amino acids 193 to 306 of SEQ ID NO:2). CUB domains are extracellular domains found in a number of functionally diverse, developmentally regulated proteins including the dorsal-ventral patterning protein tolloid, bone morphogenetic protein 1, a family of spermadhesins, complement subcomponents C1s/C1r and the neuronal recognition molecule A5. The majority of CUB domains contain four conserved cysteines which are thought to form two disulfide bridges (C₁-C₂ and C₃-C₄) (Bork et al. (1993) J. Mol. Biol. 231:539-545). The first CUB-like domain of mouse TANGO 136 (amino acids 32 to 86 of SEQ ID NO:2) includes two cysteines, and the second CUB-like domain of mouse TANGO 136 (amino acids 193 to 306 of SEQ ID NO:2) includes two cysteines. Alignments of the CUB-like domains of mouse TANGO 136 with a CUB domain consensus sequence are depicted in FIG. 8.

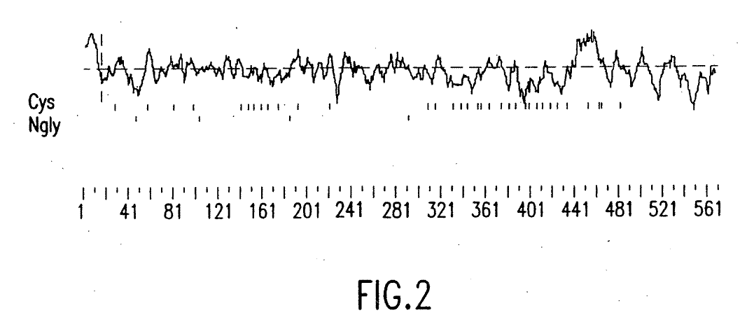

FIG. 2 depicts a hydropathy plot of a portion of mouse TANGO 136.

### Human TANGO 136

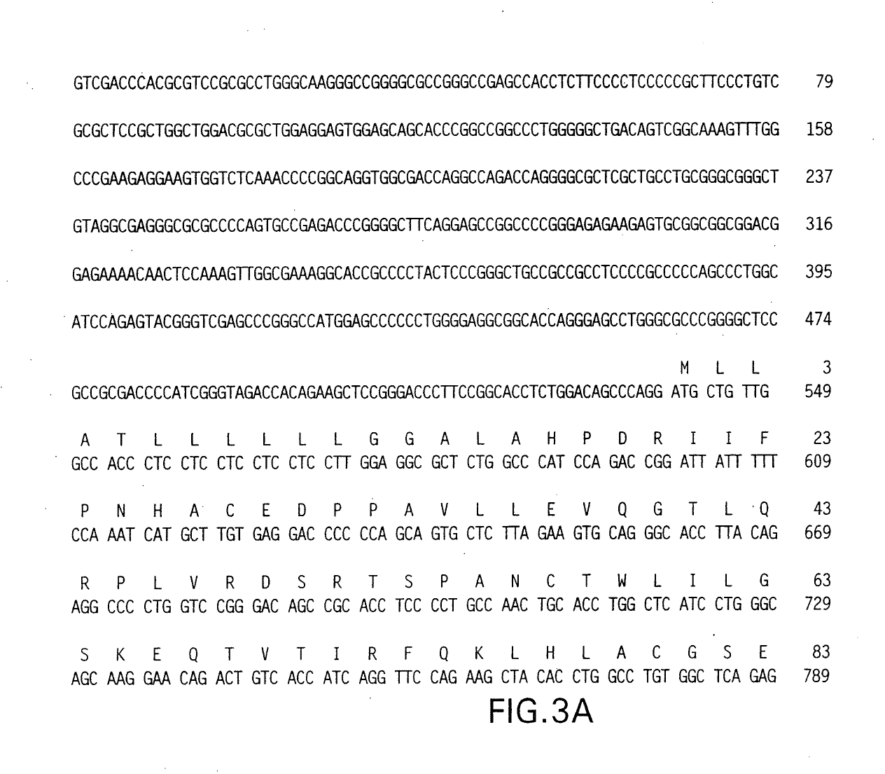

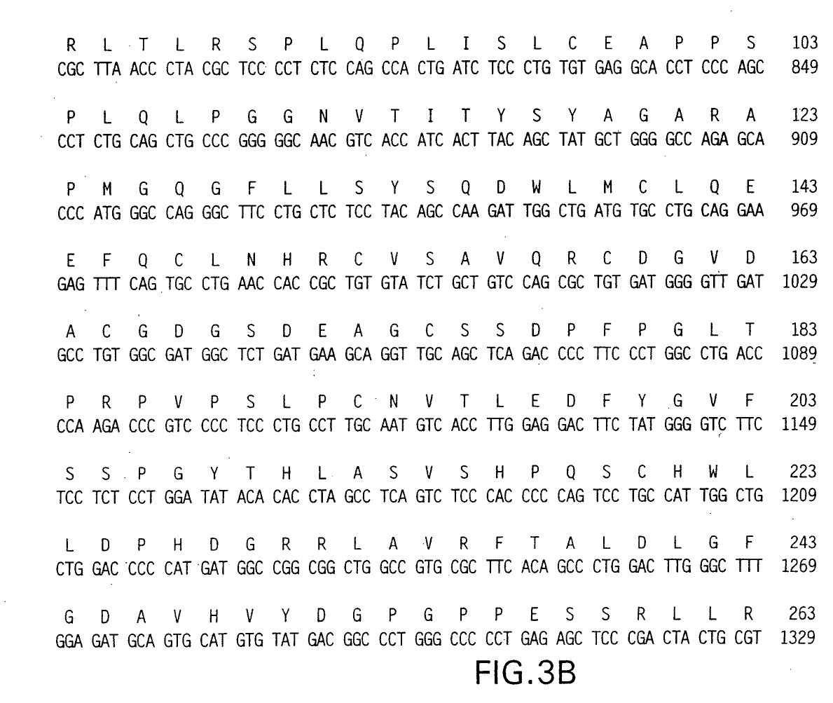

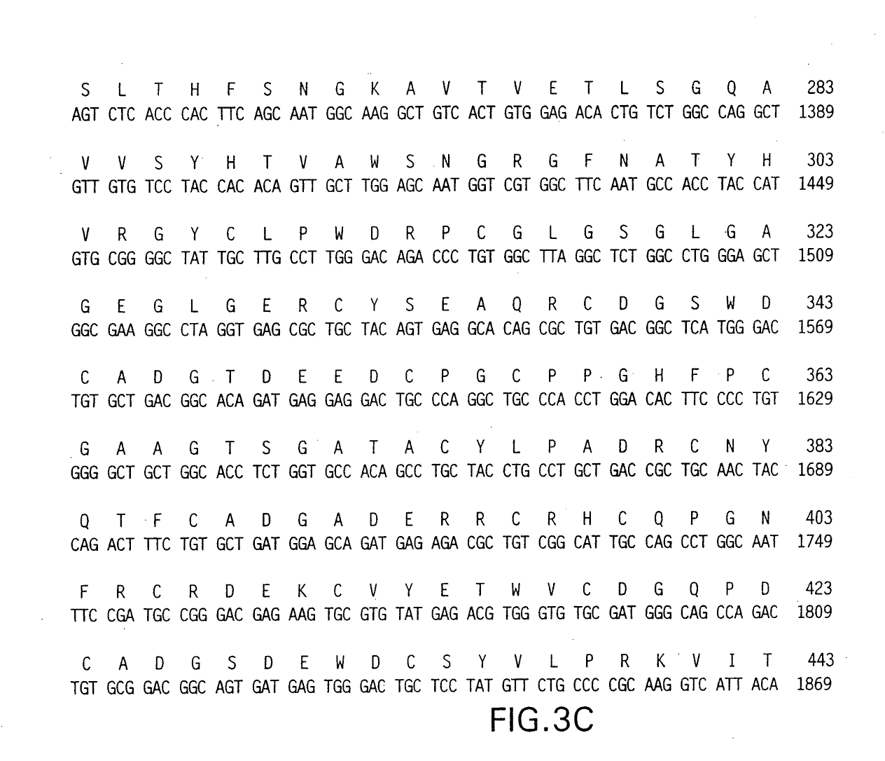

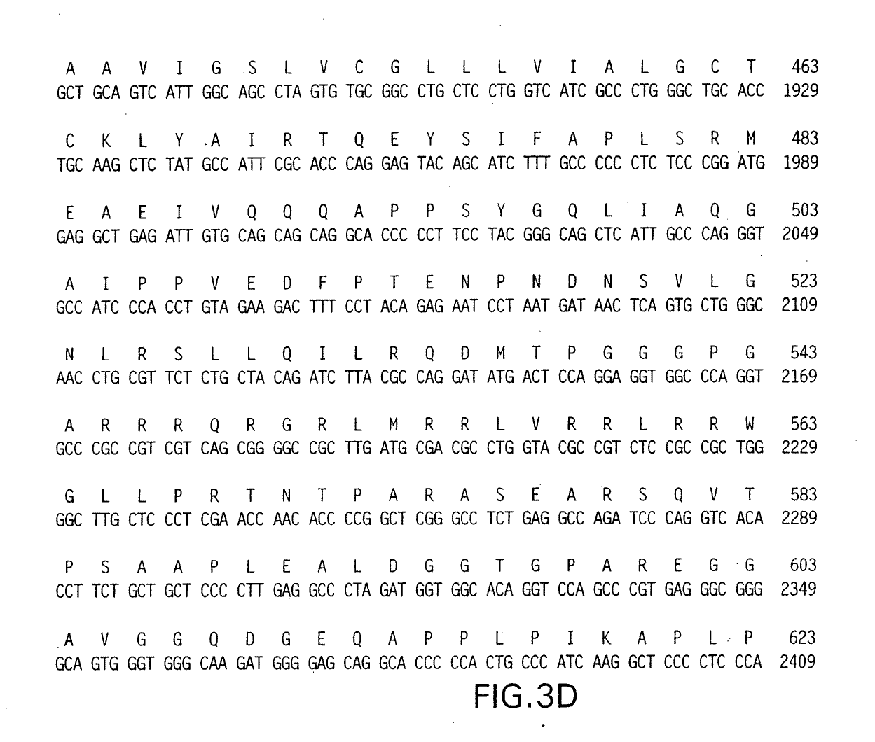

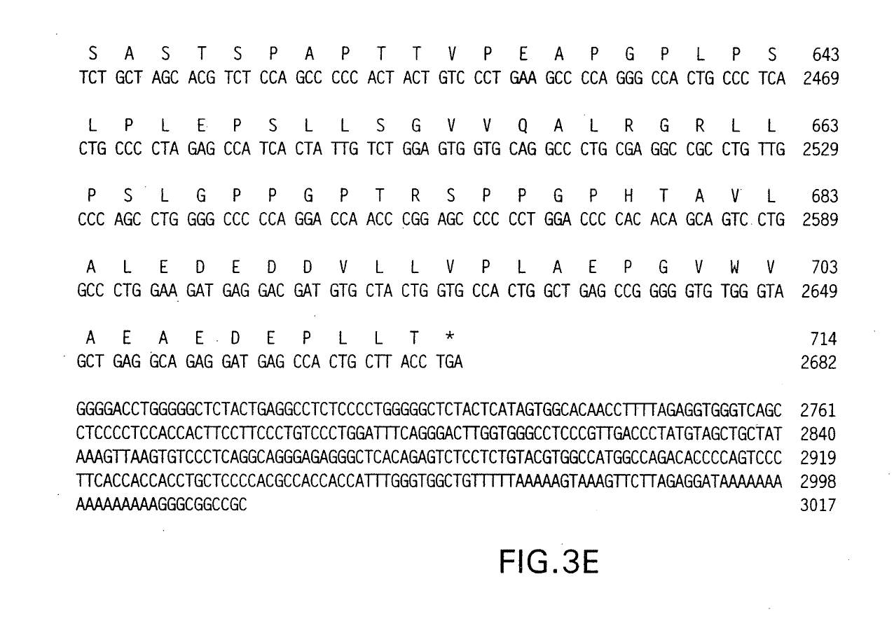

Mouse TANGO 136 cDNA described above was used to screen a human placental cDNA library to identify human clones encoding TANGO 136. One clone identified by this screening was sequenced fully. This human TANGO 136 cDNA (FIG. 3A-3E; SEQ ID NO:3) includes an open reading frame (nucleotides 541 to 2679 of SEQ ID NO:3) encoding a 713 amino acid putative type I transmembrane protein (SEQ ID NO:4).

The signal peptide prediction program SIGNALP (Nielsen et al. (1997) Protein Engineering 10:1-6) predicted that human TANGO 136 includes a 16 amino acid signal peptide (amino acid 1 to about amino acid 16 of SEQ ID NO:4) preceding the 697 amino acid mature protein (about amino acid 17 to amino acid 713 of SEQ ID NO:4). Human TANGO 136 has an extracellular domain (amino acids 17 to 440 of SEQ ID NO:4); a transmembrane domain (amino acids 441 to 461 of SEQ ID NO:4); and a cytoplasmic domain (amino acids 462 to 713 of SEQ ID NO:4).

A clone, pT136, which encodes human TANGO 136 was deposited with the American Type Culture Collection (10801 University Boulevard, Manassas, Va. 20110-2209) on Sep. 11, 1998 and assigned Accession Number 98880. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

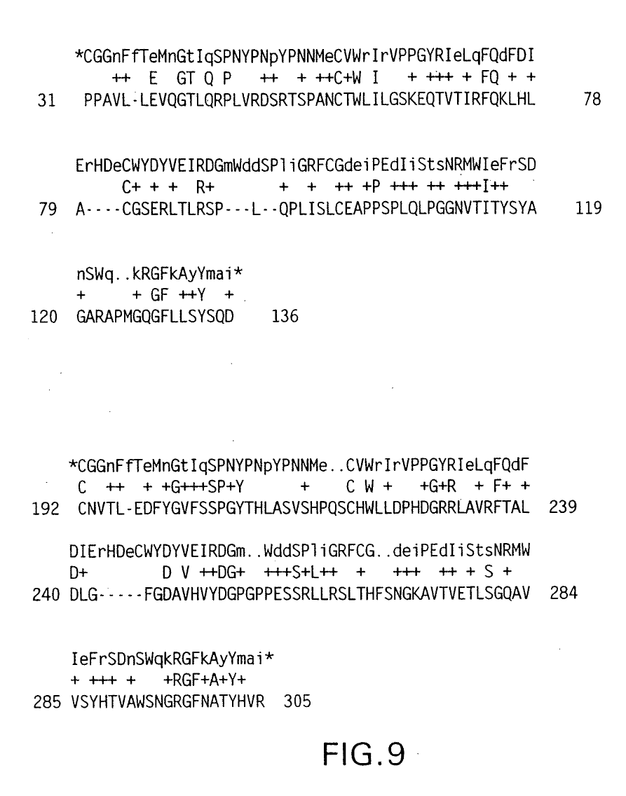

The extracellular region of human TANGO 136 includes two CUB-like domains (amino acids 31 to 136 and amino acids 192 to 305 of SEQ ID NO:4). Both of the CUB-like domains of human TANGO 136 include two cysteines. Alignments of the CUB-like domains of human TANGO 136 with a CUB domain consensus sequence are depicted in FIG. 9.

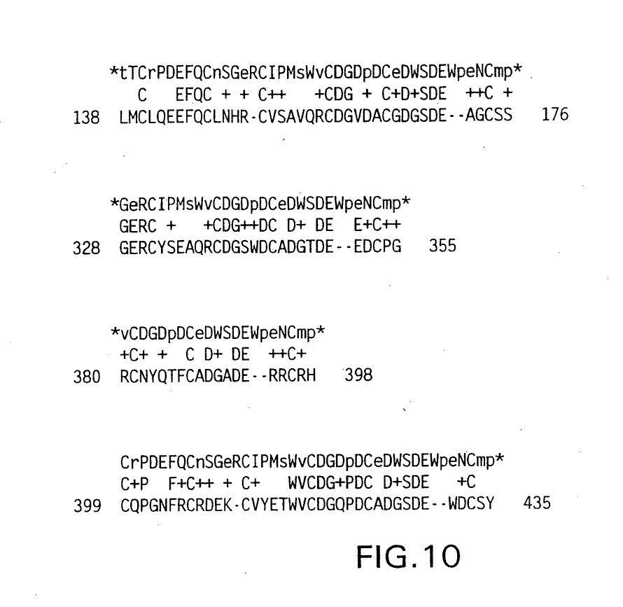

The extracellular region of human TANGO 136 also includes four LDL receptor class A domains (amino acids 138 to 176, amino acids 328 to 355; amino acids 380 to 398; and amino acids 399 to 435 of SEQ ID NO:4). The LDL receptor class A domain is an approximately 40 amino acid cysteine-rich domain having a found in LDL receptor and other members of the LDL receptor family. Repeats of this domain are thought to involved in ligand binding (Yamamoto et al. (1984) Cell 39:27-38; and Fass et al. (1997) Nature 388:691-693). The LDL receptor class A domain extending from amino acid 380 to 398 of human TANGO 136 has relatively weak homology to the consensus LDL receptor type A domain compared to the other three LDL receptor class A domains. Alignments of the LDL receptor class A domains of human TANGO 136 with a LDL receptor class A domain consensus sequence are depicted in FIG. 10.

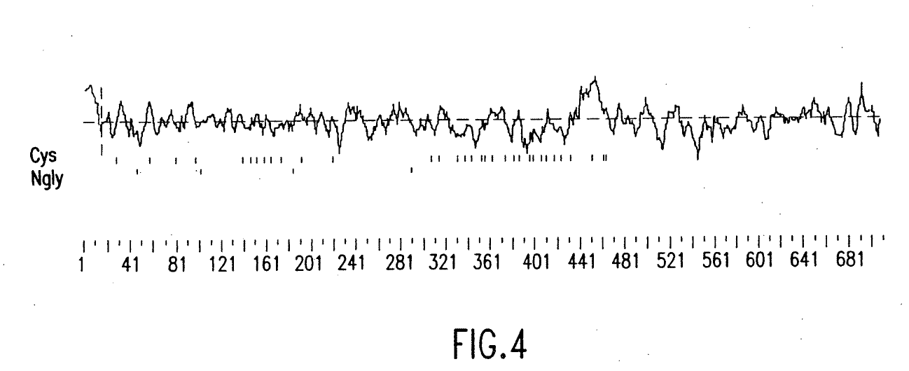

FIG. 4 depicts a hydropathy plot of human TANGO 136.

Mature human TANGO 136 has a predicted MW of 76.7 kDa (78.4 kDa for immature human TANGO 136), not including post-translational modifications.

Human TANGO 136 maps to chromosome 14 near D 14S283.

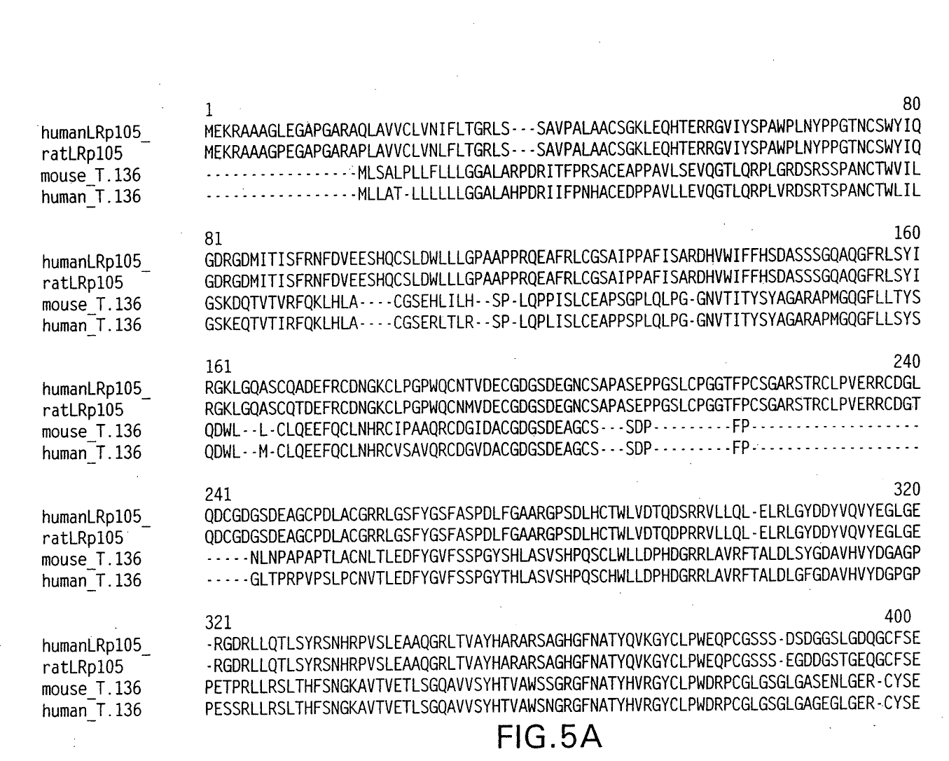

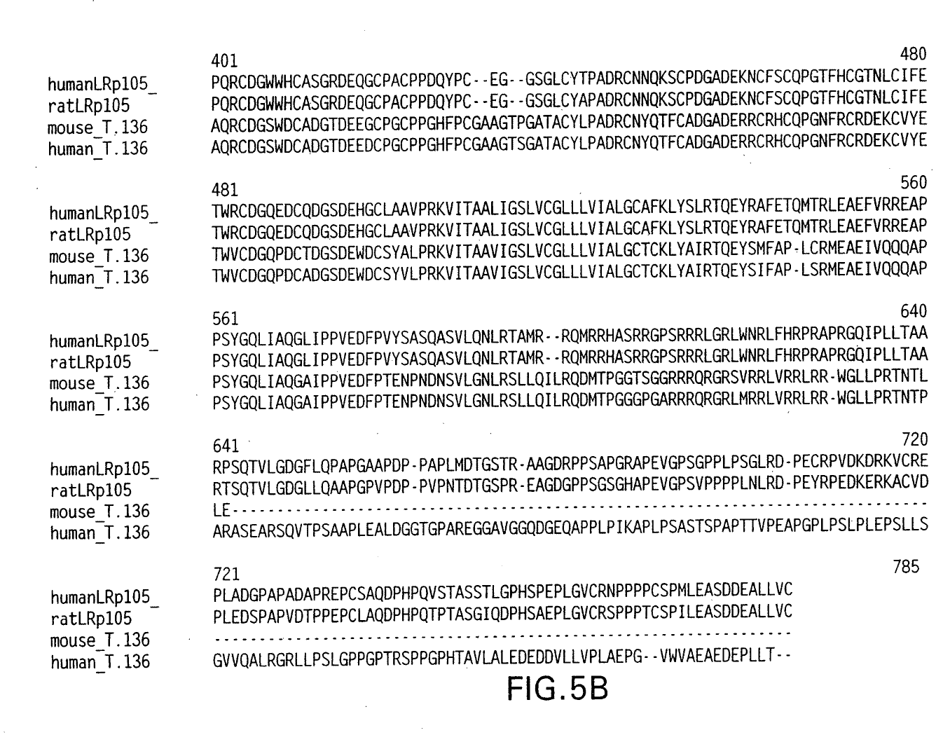

The amino acid sequence of human TANGO 136 was used to search public databases (using BLASTP; Altschul et al. (1990) J. Mol. Biol. 215:403-410) in order to identify proteins having homology to human TANGO 136. This analysis revealed that both mouse and human TANGO 136 has considerable homology to human LDL receptor related protein LRp105/LRP-3 (Ishii et al. (1998) Genomics 51:132-135). FIG. 5A-5B depicts an alignment of the amino acids sequences of mouse TANGO 136, human TANGO 136, human LRp105/LRP-3, and rat Lrp105/LRP-3.

When compared using the algorithm of Myers and Miller ((1988) CABIOS 4:11-17; PAM 120 scoring matrix, −12 gap opening penalty, −4 gap extension penalty) mouse TANGO 136 is 34.4% identical to human LRp105/LRP-3 and 34% identical to rat LRp105/LRP-3; human TANGO 136 is 38% identical to human LRp105/LRP-3 and 37.6% identical to rat Lrp105/LRP-3; and human TANGO 136 is 72.6 identical to mouse TANGO 136.

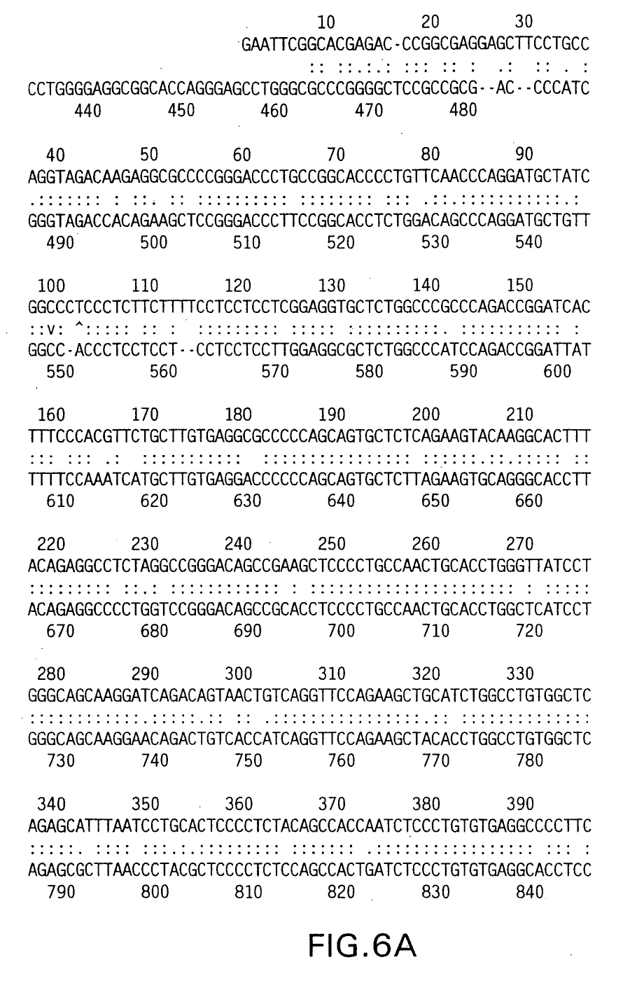

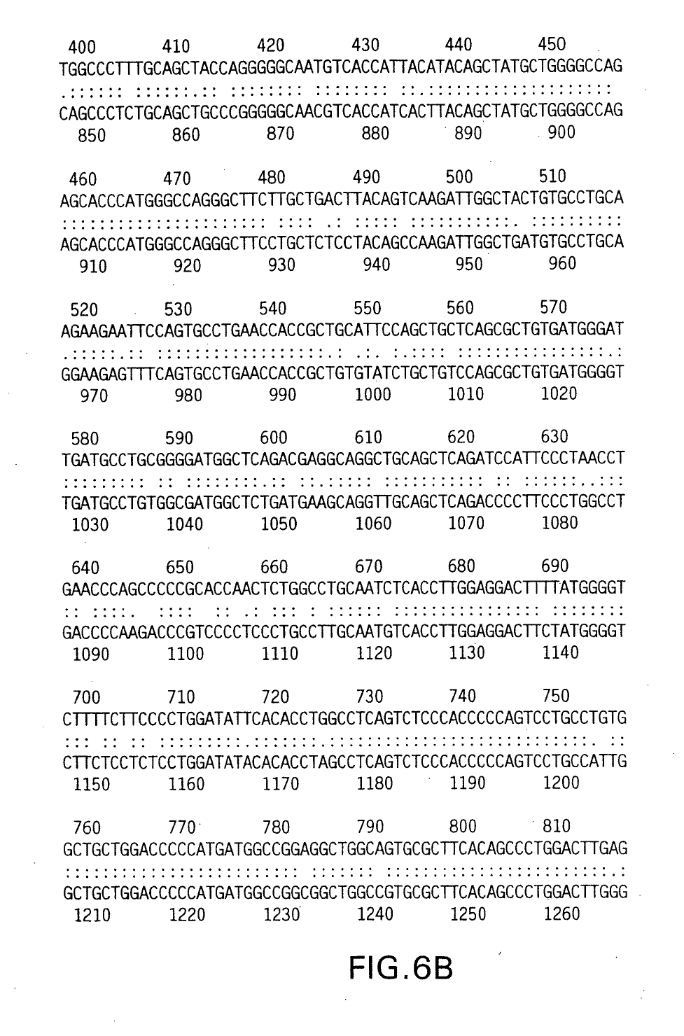

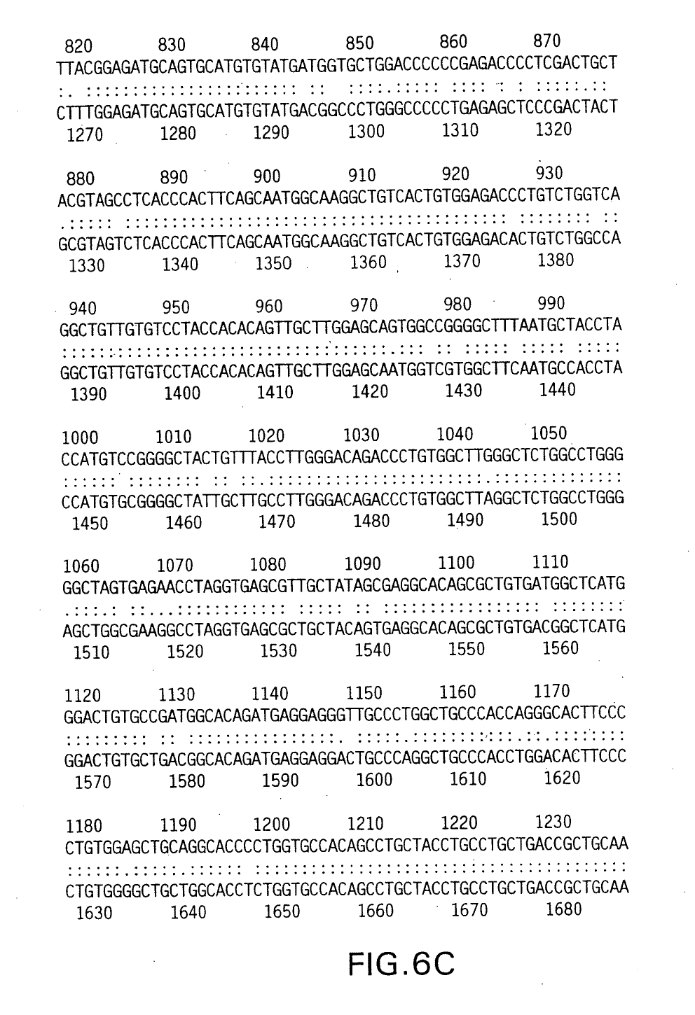

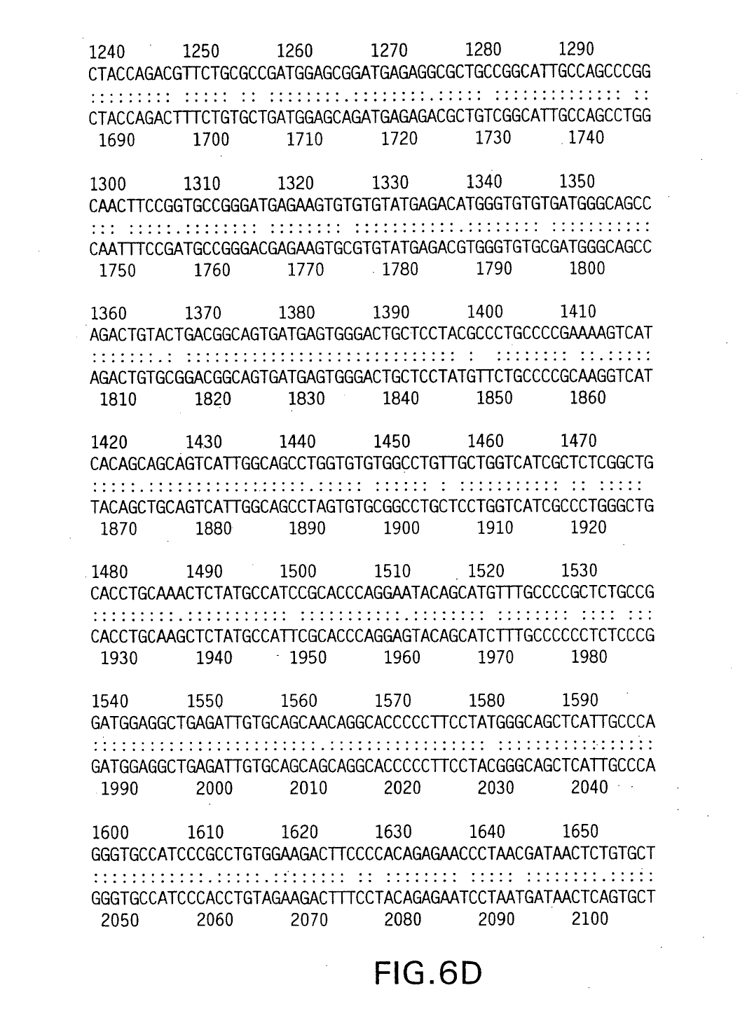

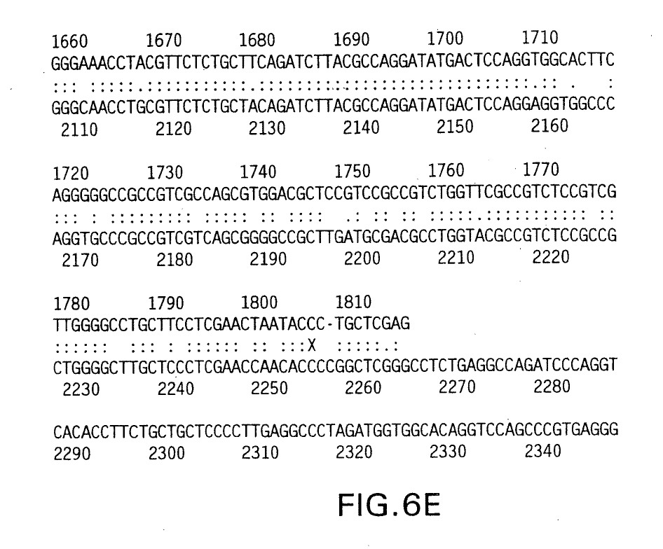

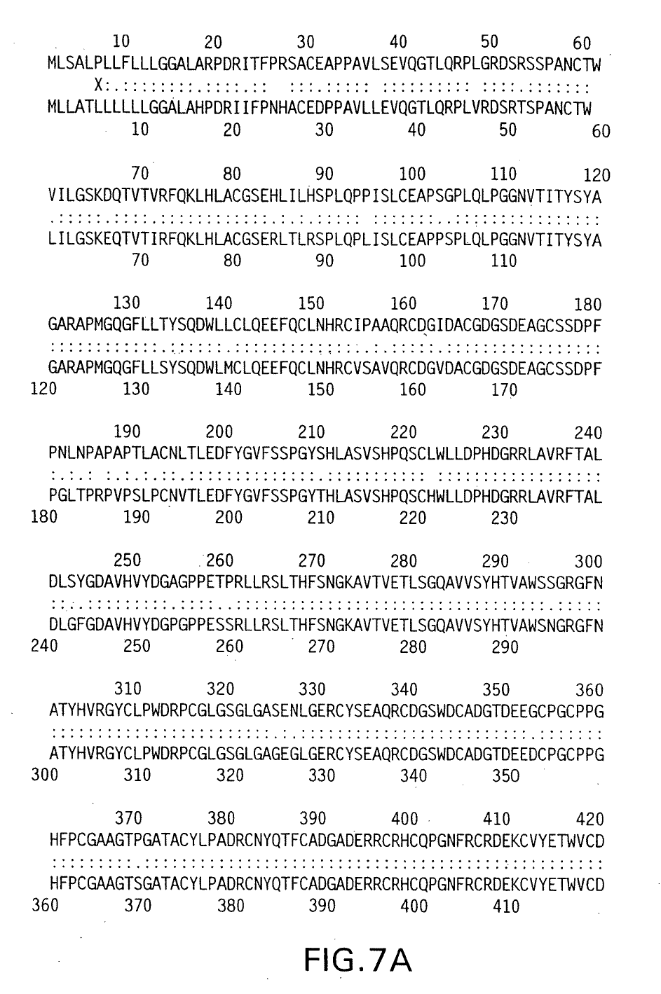

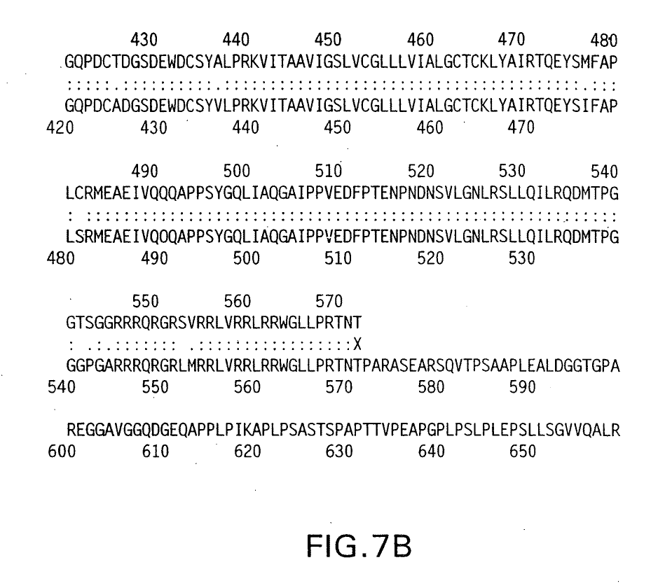

The full length human TANGO 136 nucleotide sequence is 86.1% identical (FASTA version 2.0u53; Pearson and Lipman (1988) Proc. Natl. Acad. Sci. USA 85:2444-2448) to the partial mouse TANGO 136 nucleotide sequence (FIG. 6A-6E). The full length human TANGO 136 amino acid sequence is 90.8% identical (FASTA version 2.0u53; Pearson and Lipman (1988) Proc. Natl. Acad. Sci. 85:2444-2448) to the partial mouse TANGO 136 amino acid sequence (FIG. 7A-7B). As shown in FIG. 7A-7B, the protein domain structure (described above) is highly conserved between the human and mouse proteins.

Human multiple tissue northern (MTN) blots (Clontech, Palo Alto, Calif.), containing 2 mg of poly A+ RNA per lane were probed with a mouse TANGO 136 cDNA probe. This analysis revealed that TANGO 136 mRNA is relatively highly expressed in spleen, prostate, uterus, peripheral blood leukocytes, heart, placenta, kidney and pancreas. This analysis also revealed that TANGO 136 mRNA is expressed at a somewhat lower level in thymus, testis, colon, lung, liver and skeletal muscle. TANGO 136 nucleic acids, polypeptides, agonists, and antagonists can be used to modulate the activities of the tissues in which it is expressed and thus treat disorders of these tissues. For example, TANGO 136 is expressed in prostate and testis and may be involved in spermatogenesis.

### Use of TANGO 136 Nucleic Acids, Polypeptides, and TANGO 136 Agonists or Antagonists

Due to the homology between TANGO 136 and LRp105/LRP-3, TANGO 136 is predicted to be a member of the low density lipoprotein receptor family, which includes LDLR, LRP-2 (megalin/gp330), LRP-3 (LRp105), LRP-5, LRP-6, and LR8B. Members of this family are endocytic receptors that bind and internalize ligands from the circulation and extracellular space. Since TANGO 136 is predicted to be a member of the low density lipoprotein receptor family, it may function similarly to other members of the low density lipoprotein receptor family.

LDLR binds plasma lipoproteins that contain apolipoprotein B-100 (apoB-100) or apoE on their surface. LDLR is critical for the uptake of these lipoproteins, and mutations in LDLR are the cause of familial hypercholesterolemia, a disorder characterized by high levels of cholesterol-rich LDL in the plasma. The elevation of plasma cholesterol levels in patients afflicted with familial hypercholesterolemia leads to atherosclerosis and increased risk for myocardial infarction. TANGO 136 potentially plays a role in disorders of lipoprotein metabolism and transport, e.g., cardiovascular diseases such as atherosclerosis. Accordingly, TANGO 136 nucleic acids, polypeptides and TANGO 136 antagonists and agonists are useful for treatment of disorders of lipoprotein metabolism and transport, e.g., cardiovascular diseases such as atherosclerosis.

In vitro studies have shown that LRP-2 is capable of binding and mediating the cellular uptake of a large number of different ligands including apoE-enriched very low density lipoproteins (Willnow et al. (1992) J. Biol. Chem. 267:26172-26180), complexes of urokinase plasminogen activator and plasminogen activator inhibitor-1 (tPA:PAI-1) (Willnow et al., supra), lipoprotein lipase (Willnow et al., supra), and lactoferrin. A receptor associated protein known as RAP (Orlando et al. (1992) Proc. Natl Acad. Sci. 89:6698-6702) inhibits the binding of these ligands to LRP-2. Some or all of these ligands may bind TANGO 136. Accordingly, TANGO 136 nucleic acids, polypeptides, antagonists and agonists are useful for treatment of clotting disorders, e.g., inhibiting clot formation or dissolving clots.

A few specific and physiologically relevant ligands for LRP-2 have been identified, including apolipoprotein J (apoJ)/clusterin (Kounnas et al. (1995) J. Biol. Chem. 22:13070-13075) and thyroglobulin (Zheng et al. (1998) Endocrinology 139:1462-1465). ApoJ has been reported to bind several proteins, including the bA4 peptide of the Alzheimer's precursor protein, a subclass of high density lipoprotein, and the complement membrane attack complex C5-C9 (Kounnas et al., supra). The clearance of apoj complexed with these and other molecules is expected to occur via LRP-2. Thus, LRP-2 may play an important functional role in the clearance of these complexes. For example, LRP-2 may function to target lipoproteins for clearance or may inhibit the cytolytic activity of the complement membrane C5b-C9 by clearing the apoJ/C5b-C9 complex. The fact that LRP-2 can bind the apoJ/amyloid-P complex suggests that LRP-2 may be involved in regulating the pathogenesis of Alzheimer's disease. A role for LRP-2 in Alzheimer's disease is further supported by another study that showed that LRP-2 may be involved in transporting the apoJ/amyloid-P complex across the blood-brain-barrier (Zlokovic et al. (1996) Proc. Natl. Acad. Sci. 93:4229-4234). Thus, TANGO 136 nucleic acids, proteins, agonists, and antagonists are useful for the treatment of Alzheimer's disease and other neurodegenerative disorders, e.g., Huntington's disease and Parkinson's disease.

LRP-2 is involved in participating in the endocytosis of thyroglobulin, which results in the release of thyroid hormones (Zheng et al. (1998) Endocrinolgy 139:1462-65). TANGO 136 may also be involved in the regulating the release of thyroid hormones. Thus, TANGO 136 nucleic acids, proteins, agonists, and antagonists are useful for the treatment of thyroid disorders, e.g., thyroid hormone release disorders.

LRP-2 is also predicted to play a role as a drug receptor and is thought to be involved in the uptake of polybasic drugs, e.g., aprotinin, aminoglycosides and polymyxin B. The uptake of polybasic drugs can be toxic, e.g., the administration of aminoglycosides is often associated with nephro- and ototoxicity. TANGO 136 may also mediate uptake of polybasic drugs, and TANGO 136 nucleic acids, proteins, agonists, and antagonists are useful for the modulating the uptake of such drugs. TANGO 136 can also be used to design less toxic versions of such drugs.

In addition, LRP-2 is involved in the pathogenesis of Heymann Nephritis nephropathy (HN), an autoimmune glomerular disease, which is similar to human membranous nephropathy. It is thought that LRP-2 is the major pathogenic antigen and forms an antigen-antibody complex between the glomular basement membrane and the foot processes of glomerular epithelial cells. The presence of the antigen-antibody complex leads to extensive damage of the basement membrane and proteinuria (Farquhar et al. (1994) Ann. N.Y. Acad. Sci. 97-106). Similar to LRP-2, TANGO 136 may play a pathogenic role in autoimmune glomerular disease. Thus, TANGO 136 nucleic acids, proteins, agonists, and antagonists are useful for the treatment of autoimmune glomerular disease.

LRP-5 and LRP-6 are thought to function in endocytosis. Based on genetic evidence, LRP-5 and possibly LRP-6 are thought to play a role in the molecular pathogenesis of type I diabetes (Brown et al. (1998) Biochem. Biophys. Res. Comm. 248:879-888). TANGO 136 is also likely plays a role in type I diabetes. Thus, TANGO 136 nucleic acids, proteins, agonists, and antagonists are useful for the treatment of type I diabetes.

LR8B is expressed in brain and might be involved in brain-specific lipid transport. Brain-specific lipid transport may involve apoE4, which is associated with Alzheimer's disease. TANGO 136 may also be involved in brain-specific lipid transport, and TANGO 136 nucleic acids, proteins, agonists, and antagonists are useful for the treatment of Alzheimer's disease.

In general, TANGO 136 nucleic acids, proteins, agonists, and antagonists may be useful for the treatment of neurological disorders, e.g., neurodegenerative disorders and neuropsychiatric disorders. Examples of neurodegenerative disorders include Alzheimer's disease, Parkinson's disease, and Huntington's disease. Examples of neuropsychiatric disorders include schizophrenia, attention deficit disorder, unipolar affective (mood) disorder, bipolar affective (mood) disorders (e.g., severe bipolar affective disorder (BP-I) and bipolar affective disorder with hypomania and major depression (BP-II)), and schizoaffective disorders.

### TANGO 128

In one aspect, the present invention is based on the discovery of cDNA molecules which encode a novel family of proteins having sequence identity to vascular endothelial growth factor (VEGF), referred to herein as TANGO 128 proteins.

For example, the VEGF family to which the TANGO 128 proteins of the invention bear sequence identity, are a family of mitogens which contain a platelet-derived growth factor (PDGF) domain having conserved cysteine residues. These cysteine residues form intra- and inter-chain disulfide bonds which can affect the structural integrity of the protein. Thus, included within the scope of the invention are TANGO 128 proteins having a platelet-derived growth factor (PDGF) domain. As used herein, a PDGF-domain refers to an amino acid sequence of about 55 to 80, preferably about 60 to 75, 65 to 70, and more preferably about 69 amino acids in length. A PDGF domain of TANGO 128 extends, for example, from about amino acids 269 to 337 of SEQ ID NO:6.

Conserved amino acid motifs, referred to herein as “consensus patterns” or “signature patterns”, can be used to identify TANGO 128 family members (and/or PDGF family members) having a PDGF domain. For example, the following signature pattern can be used to identify TANGO 128 family members: P-x-C-[LV]-x(3)-R-C-[GSTA]-G-x(0, 3)-C-C. The signature patterns or consensus patterns described herein are described according to the following designation: all amino acids are indicated according to their universal single letter designation; “x” designates any amino acid; x(n) designates n number of amino acids, e.g., x (2) designates any two amino acids, e.g., x (1, 3) designates any of one to three amino acids; and, amino acids in brackets indicates any one of the amino acids within the brackets, e.g., [LV] indicates any of one of either L (leucine) or V (valine). TANGO 128 has such a signature pattern at about amino acids 272 to 287 of SEQ ID NO:6.

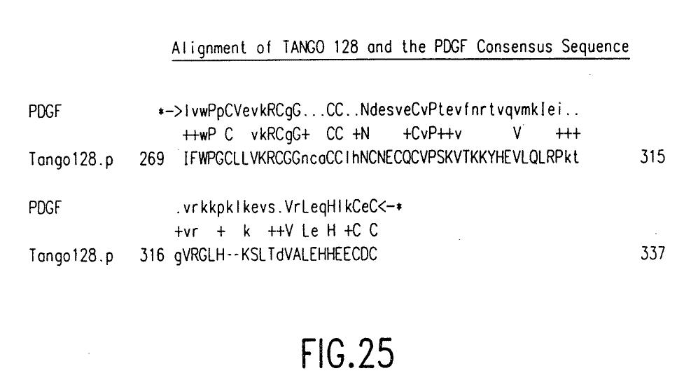

A PDGF domain further contains at least about 2 to 10, preferably, 3 to 9, 4 to 8, or 6 to 7 conserved cysteine residues. By alignment of a TANGO 128 family member with a PDGF consensus sequence, conserved cysteine residues can be found. For example, as shown in FIG. 25, there is a first cysteine residue in the PDGF consensus sequence that corresponds to a cysteine residue at amino acid 274; there is a second cysteine residue in the PDGF consensus sequence that corresponds to a cysteine residue at amino acid 280 of TANGO 128; there is a third cysteine residue in the PDGF consensus sequence that corresponds to a cysteine residue at amino acid 286 of TANGO 128; there is a fourth cysteine residue in the PDGF consensus sequence that corresponds to a cysteine residue at amino acid 287 of TANGO 128; there is a fifth cysteine residue in the PDGF consensus sequence that corresponds to a cysteine residue at amino acid 296 of TANGO 128; there is a sixth cysteine residue in the PDGF consensus sequence that corresponds to a cysteine residue at amino acid 335 of TANGO 128; and/or there is a seventh cysteine residue in the PDGF consensus sequence that corresponds to a cysteine residue at amino acid 337 of TANGO 128. The PDGF consensus sequence is also available from the HMMer version 2.0 software as Accession Number PF00341. Software for HMM-based profiles is available from http://www.csc.ucsc.edu/research/compbio/sam.html and from http://genome.wustl.edu/eddy/hmmer.html.

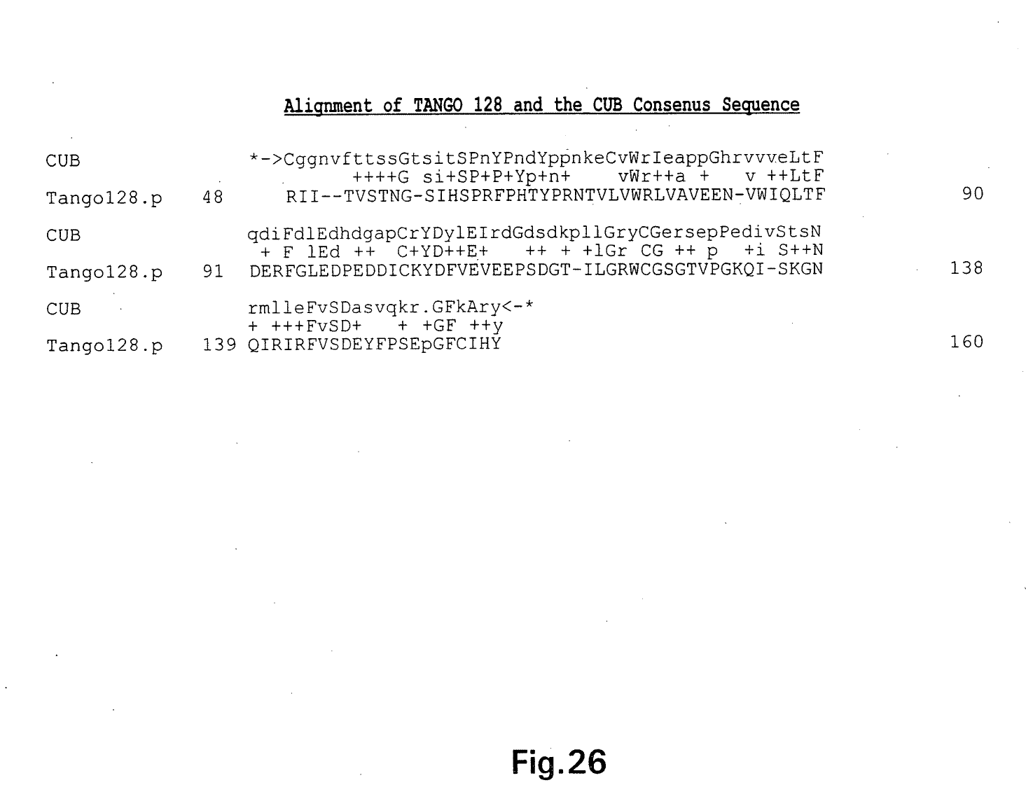

The present invention also features TANGO 128 proteins having a CUB domain. The CUB domain is associated with various developmentally regulated proteins and as such is likely to be involved in developmental processes. As used herein, a CUB domain refers to an amino acid sequence of about 90 to about 140, preferably about 100 to 125, 110 to 115, and more preferably about 113 amino acids in length. A CUB domain of TANGO 128 extends, for example, from about amino acids 48 to 160 of SEQ ID NO:6. An alignment of TANGO 128 and the CUB consensus sequence is shown in FIG. 26.

Conserved amino acid motifs, referred to herein as “consensus patterns” or “signature patterns”, can be used to identify TANGO 128 family members having a CUB domain. For example, the following signature pattern can be used to identify TANGO 128 family members: GS-x (3, 11)-[ST]-[PLYA]-x(2)-P-x (2,3)-Y-x (6, 8)-[WY]-x (9, 11)-[LVIF]-x-[LIF]-x (7,10)-C. TANGO 128 has such a signature pattern at about amino acids 56 to 104 of SEQ ID NO:6.

A CUB domain further contains at 2 or more conserved cysteine residues which are likely to form disulfide bonds which affect the structural integrity of the protein. Also included within the scope of the present invention are TANGO 128 proteins having a signal sequence.

In certain embodiments, a TANGO 128 family member has the amino acid sequence of SEQ ID NO:2, and the signal sequence is located at amino acids 1 to 20, 1 to 21, 1 to 22, 1 to 23 or 1 to 24. In such embodiments of the invention, the domains and the mature protein resulting from cleavage of such signal peptides are also included herein. For example, the cleavage of a signal sequence consisting of amino acids 1 to 22 results in a mature TANGO 128 protein corresponding to amino acids 23 to 345. The signal sequence is normally cleaved during processing of the mature protein.

In one embodiment, a TANGO 128 protein of the invention includes a PDGF domain and/or a CUB domain. In another embodiment, a TANGO 128 protein of the invention includes a PDGF domain, a CUB domain, a signal sequence, and is secreted.

### Human TANGO 128

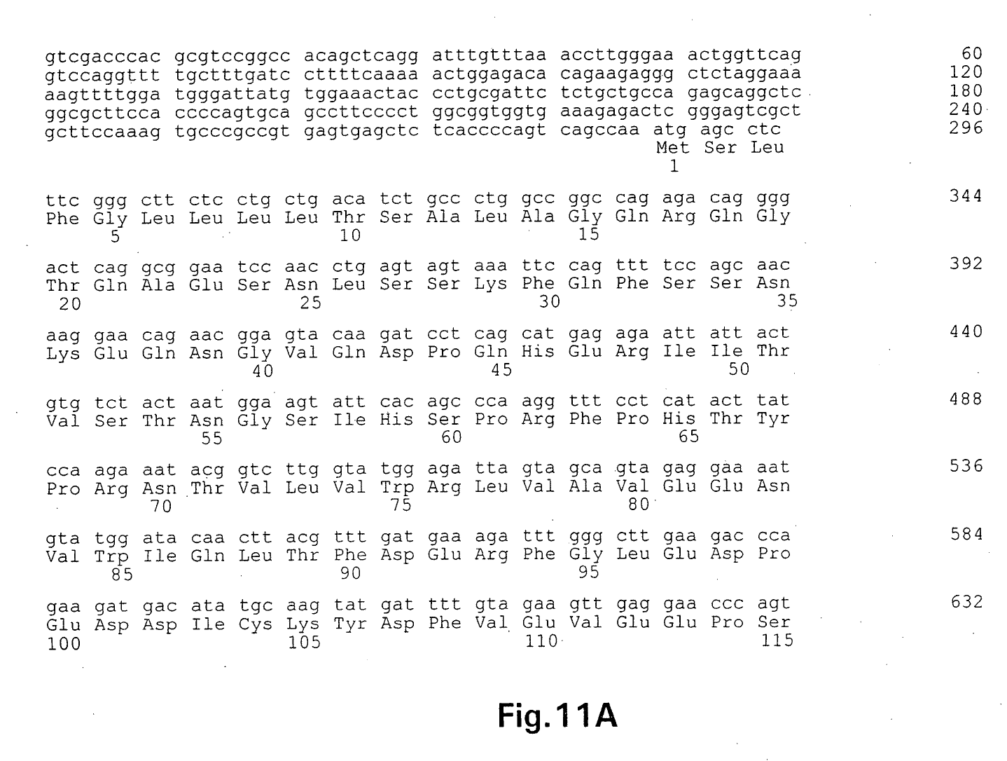

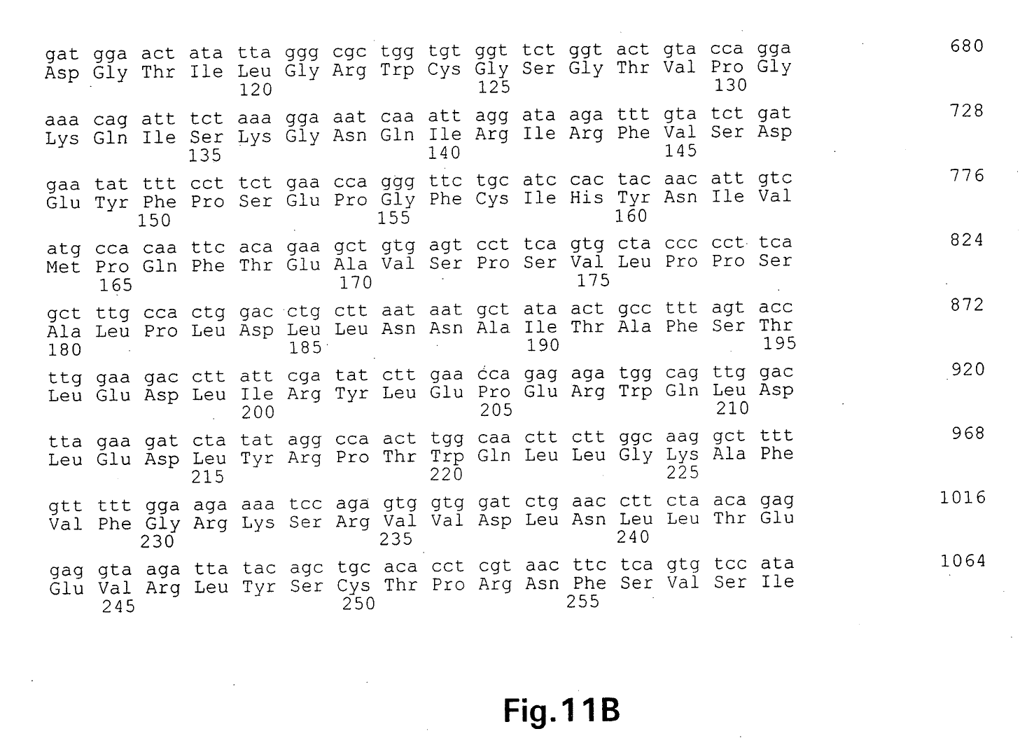

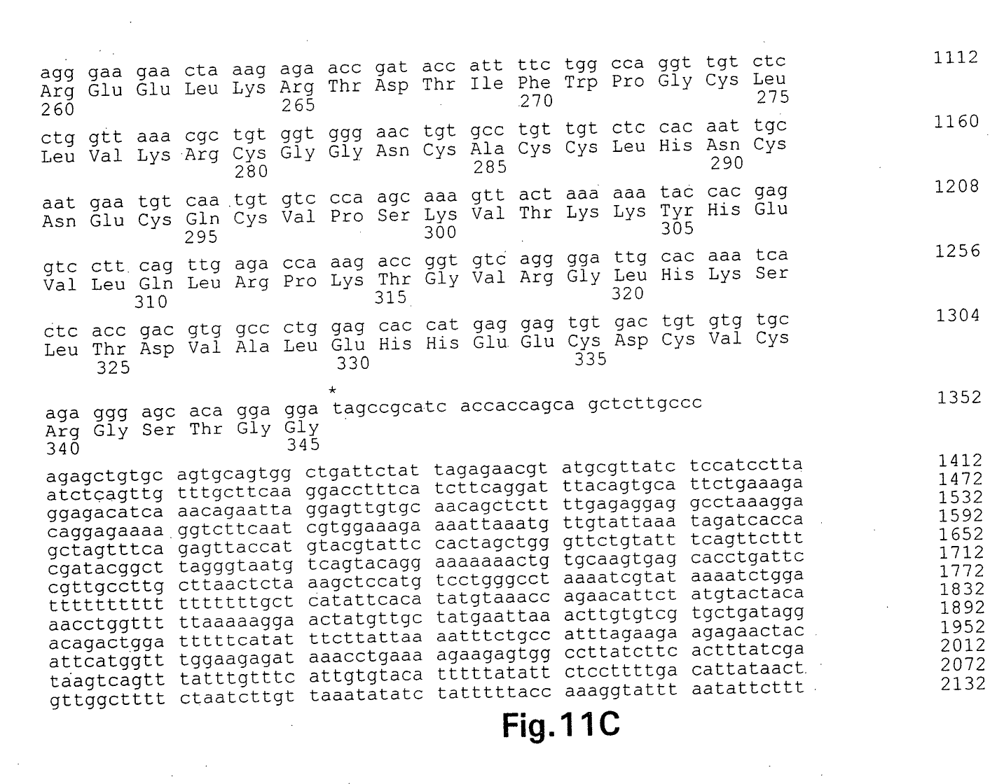

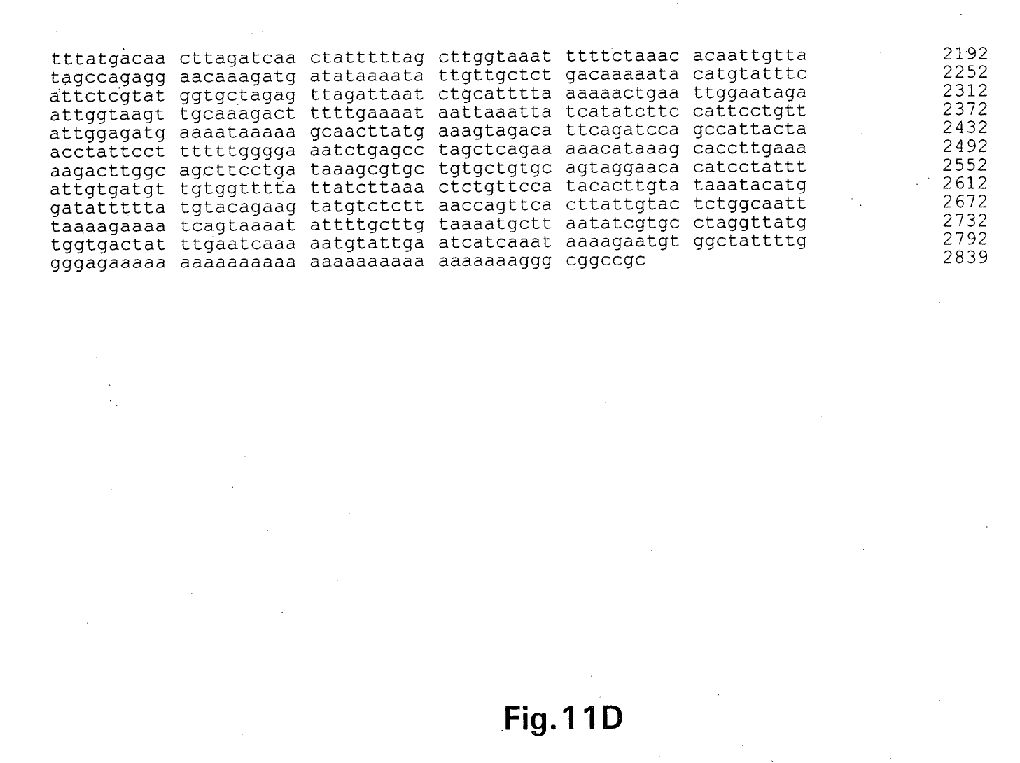

The cDNA encoding human TANGO 128 was isolated by homology screening. Briefly, a clone encoding a portion of TANGO 128 was identified through high throughput screening of a mesangial cell library and showed homology to the VEGF family. An additional screen of the mesangial cell library was performed to obtain a clone comprising full length human TANGO 128. Human TANGO 128 includes a 2839 nucleotide cDNA (FIG. 11A-11D; SEQ ID NO:5). It is noted that the nucleotide sequence depicted in SEQ ID NO:5 contains SalI and NotI adapter sequences on the 5′ and 3′ ends, respectively (5′GTCGACCCACGCGTCCG 3′, and 5′ GGGCGGCCGC 3′). Thus, it is to be understood that the nucleic acid molecules of the invention include not only those sequences with such adaptor sequences but also the nucleic acid sequences described herein lacking the adaptor sequences. The open reading frame of this cDNA (nucleotides 288 to 1322 of SEQ ID NO:5) encodes a 345 amino acid secreted protein (SEQ ID NO:6).

The signal peptide prediction program SIGNALP (Nielsen et al. (1997) Protein Engineering 10:1-6) predicted that human TANGO 128 includes a 22 amino acid signal peptide (amino acids 1 to amino acid 22) preceding the mature TANGO 128 protein (corresponding to amino acid 23 to amino acid 345).

Human TANGO 128 includes a PDGF domain from about amino acids 269 to 337. Human TANGO 128 further includes a CUB domain (about amino acids 48 to 160).

A clone, EpDH237, which encodes human TANGO 128 was deposited as part of EpDHMix1 with the American Type Culture Collection (ATCC®, 10801 University Boulevard, Manassas, Va. 20110-2209) on Nov. 20, 1998 which was assigned Accession Number 98999. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

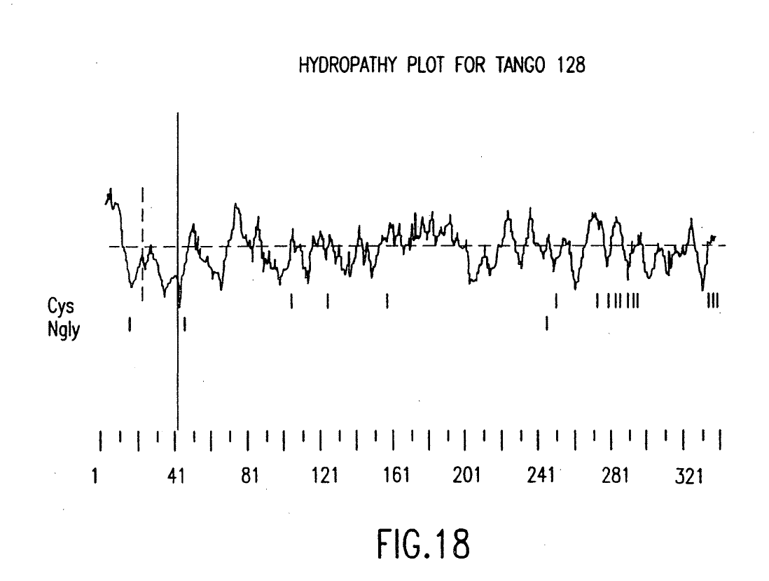

FIG. 18 depicts a hydropathy plot of human TANGO 128. The hydrophobic region at the beginning of the plot which corresponds to about amino acids 1 to 22 is the signal sequence of TANGO 128.

Northern analysis of human TANGO 128 mRNA expression revealed the presence of approximately a 3.8 kb transcript that is expressed in a wide range of tissues including heart, brain, placenta, lung, liver, skeletal muscle, kidney, pancreas, spleen, prostate, testis, ovary, small intestine, colon, and peripheral blood leukocytes. The highest levels of expression were seen in the pancreas, kidney and ovary. An additional TANGO 128 transcript of approximately 3 kb is seen in the ovary, prostate, pancreas, and kidney.

The human gene for TANGO 128 was mapped on radiation hybrid panels to the long arm of chromosome 4, in the region q28-31. Flanking markers for this region are WI-3936 and AFMCO27ZB9. The FGC (fibrinogen gene cluster), GYP (glycophorin cluster), IL15 (interleukin 15), TDO2 (tryptophan oxygenase), and MLR (mineralcorticoid receptor) genes also map to this region of the human chromosome. This region is syntenic to mouse chromosome 8. The Q (quinky), pdw (proportional dwarf), and lyl1 (lymphoblastomic leukemia) loci also map to this region of the mouse chromosome. Il15 (interlukin 15), mlr (mineral corticoid receptor), ucp (uncoupling protein), and clgn (calmegin) genes also map to this region of the mouse chromosome.

TANGO 128 protein binds to endothelial cells with high affinity: In vitro studies of AP-T128 binding to bACE cells (bovine adrenal cortical capillary endothelial cells) were performed with Phospha-Light chemiluminescent assay system (Tropix, Inc. Bedford, Mass.). bACE cells were plated into gelatinized 96-well plates (3000 cells/well) and allowed to grow to confluency. The cells were then fixed with acetone. AP-hT128 was incubated with the cells for 1 hour. Specific binding was detected with a microplate luminometer according to the manufacturer's instruction.

The binding studies indicated high affinity to bovine adrenal capillary endothelial cells in culture. Half-maximal binding occurred with approximately 0.5 nM AP-T128. AP-T128 was capable of exhibiting binding to adrenal cortex, ovary (medulla), mucosal layer of colon, and bronchial epithelium of lung in the mouse.

Recombinant TANGO 128 protein stimulates endothelial cell proliferation In vitro: The ability of A1 protein to stimulate the growth of endothelial cells was tested by bovine adrenal capillary endothelial (bACE) cell proliferation assay. Briefly, cultured bovine capillary endothelial cells dispersed with 0.05% trypsin/0.53 mM EDTA were plated onto gelatinized (Difco) 24-well culture plates (12,500 cell/well) in DMEM containing 10% bovine calf serum (BCS) and incubated for 24 hours. The media was replaced with 0.5 ml DMEM containing 5% bovine calf serum and either buffer only or buffer containing AP-hT128 were added. After 72 hours, the cells were counted with Coulter Counter. By cell count, there is a modest increase in bACE cells after 3 days. TANGO 128 was shown to exhibit proliferative activity on endothelial cells In vitro. Preliminary studies show that AP-T128 has mitogenic activity on primary bovine adrenal cortical capillary endothelial cells (bACE cells).

### Mouse TANGO 128

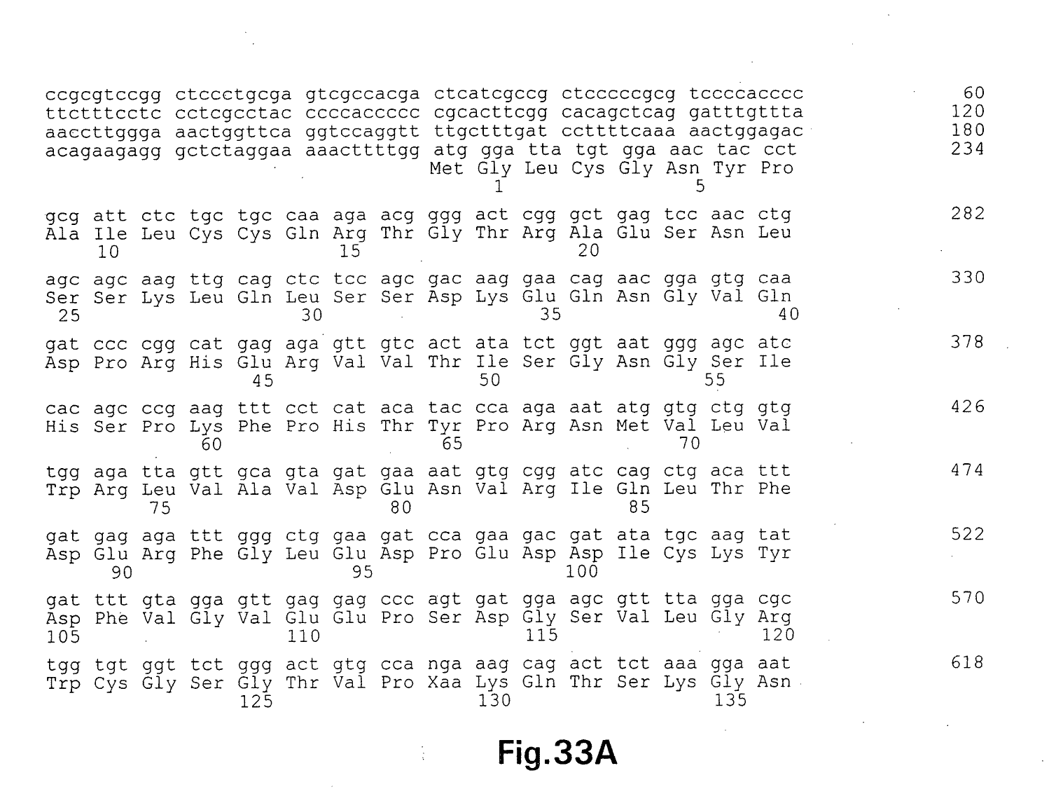

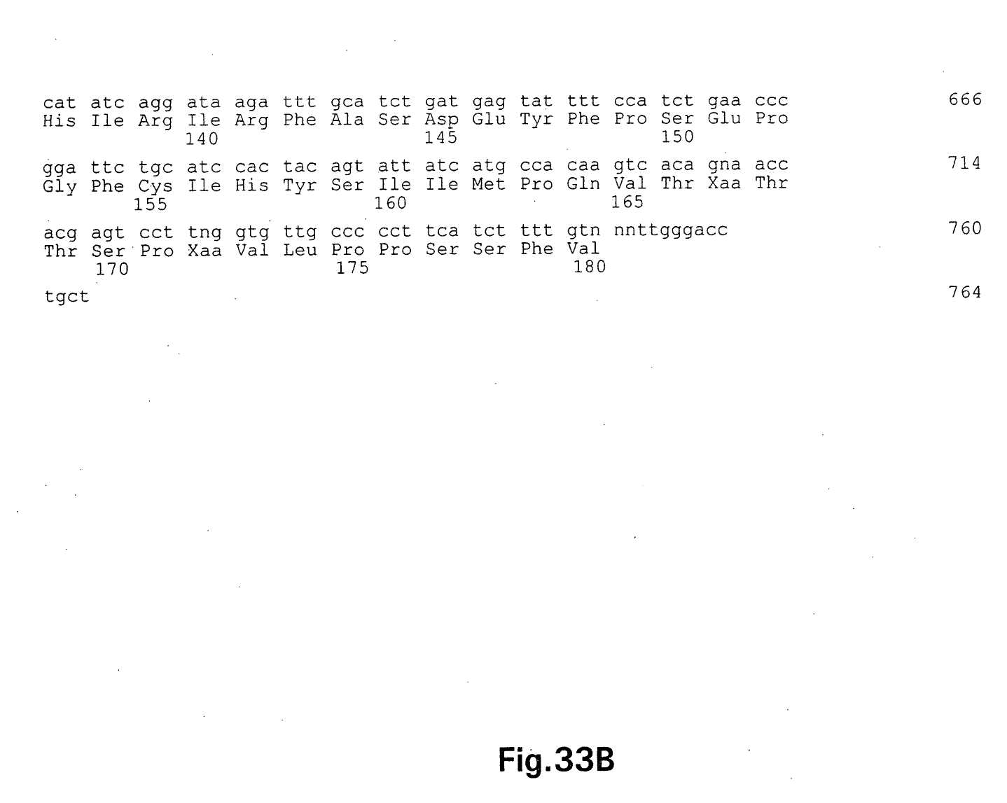

A mouse homolog of human TANGO 128 was identified. A cDNA encoding mouse TANGO 128 was identified by analyzing the sequences of clones present in a mouse osteoblast lipopolysaccharide (LPS) stimulated cDNA library. This analysis led to the identification of a clone, jtmoa114h01, encoding full-length mouse TANGO 128. The mouse TANGO 128 cDNA of this clone is 764 nucleotides long (FIG. 33A-33B; SEQ ID NO: 19). It is noted that the nucleotide sequence contains Sal I and Not I adapter sequences on the 5′ and 3′ ends, respectively. The open reading frame of this cDNA (nucleotides 211 to 750 of SEQ ID NO:19) encodes a 179 amino acid secreted protein (SEQ ID NO:20).

In one embodiment of a nucleotide sequence of mouse TANGO 128, the nucleotide at position 595 is a guanine (G). In this embodiment, the amino acid at position 129 is glycine (G). In another embodiment of a nucleotide sequence of mouse TANGO 128, the nucleotide at position 595 is a cytosine (C). In this embodiment, the amino acid at position 129 is arginine (R). In another embodiment of a nucleotide sequence of mouse TANGO 128, the nucleotide at position 595 is a thymidine (T). In this embodiment, the amino acid at position 129 is a stop codon (Opal) and results in a polypeptide of 128 aa in length.

In one embodiment of a nucleotide sequence of mouse TANGO 128, the nucleotide at position 710 is a thymidine (T). In this embodiment, the amino acid at position 167 is valine (V). In another embodiment of a nucleotide sequence of mouse TANGO 128, the nucleotide at position 710 is a cytosine (C). In this embodiment, the amino acid at position 167 is alanine (A). In another embodiment of a nucleotide sequence of mouse TANGO 128, the nucleotide at position 710 is adenine (A). In this embodiment, the amino acid at position 167 is glutamine (E). In another embodiment of a nucleotide sequence of mouse TANGO 128, the nucleotide at position 710 is guanine (G). In this embodiment, the amino acid at position 167 is glycine (G).

In one embodiment of a nucleotide sequence of mouse TANGO 128, the nucleotide at position 725 is a thymidine (T). In this embodiment, the amino acid at position 172 is leucine (L). In another embodiment of a nucleotide sequence of mouse TANGO 128, the nucleotide at position 725 is a cytosine (C). In this embodiment, the amino acid at position 172 is serine (S). In another embodiment of a nucleotide sequence of mouse TANGO 128, the nucleotide at position 725 is a adenine (A). In this embodiment, the amino acid at position 172 is a stop codon (Amber) and results in a polypeptide of 171 aa in length. In another embodiment of a nucleotide sequence of mouse TANGO 128, the nucleotide at position 725 is a guanine (G). In this embodiment, the amino acid at position 172 is tryptophan.

In situ tissue screening was performed on mouse adult and embryonic tissue to analyze the expression of mouse TANGO 128 mRNA. Of the tissues tested, expression in the adult mouse was highest in the reproductive tract, testes and ovary.

In the case of adult expression, the following results were obtained: For the testis, a signal outlining some seminiferous tubules was detected which possibly included the lamina propria which contains fibromyocytes (myoid cells). In the placenta, a signal was detected in the labyrinthine tissue. In the ovaries, a strong, multifocal signal was detected. A weak signal was detected from the capsule of the adrenal gland. In the spleen, a ubiquitous signal was detected which was slighter higher in the non-follicular spaces. A weak, ubiquitous signal was detected in the submandibular gland. Weak expression was also seen in a number of other tissues. For example, a very weak signal was detected in the olfactory bulb of the brain. A very weak ubiquitous signal only slightly above background was detected in the colon, small intestine, and liver. A multifocal signal was detected in brown and white fat. No signal was detected in the following tissues: eye and harderian gland, spinal cord, stomach, thymus, skeletal muscle, bladder, heart, lymph node, lung, pancreas, and kidney.

Embryonic expression was seen in a number of tissues. The highest expressing tissue was the capsule of the kidney which was seen at E14.5 and continues to P1.5. Adult kidney did not show this expression pattern. Other tissues with strong expression include the frontal cortex and developing cerebellum of the brain, various cartilage structures of the head including Meckel's cartilage and the spinal column. Numerous tissues with a smooth muscle component also showed expression including the small intestine and stomach as well as the diaphragm at early embryonic stages, E13.4 and E14.5. At E13.5, signal in the brain was seen in areas adjacent to the ventricles, which includes the roof of the midbrain and the roof of the neopallial cortex. A stronger signal was observed from the skin of the snout and follicles of vibrissae extending to the epithelium of the mouth and tongue. A diffuse signal around developing clavicle, hip, and vertebrae was suggestive of muscle expression. A signal did not appear to be expressed from developing bone or cartilage except in the case of the spinal column where there may have been some cartilage expression. Large airways of the lung were positive as is the diaphragm, stomach and intestines. A signal from the digestive tract appeared to be associated with smooth muscle. At E14.5, the expression pattern was nearly identical to that seen at E13.5 except kidney expression was now apparent. Signal was restricted to the capsule and was the strongest expressing tissue. The capsule of the adrenal gland had expression but to a lesser extent than that seen in the kidney. The developing musculature of the feet had strong expression as well. At E16.5, signal in the muscle and skin was decreased. Diaphragm expression was no longer apparent but the smooth muscle of the intestine was still seen. Strongest signal was seen in the skin and muscle of the snout and feet, capsule of the kidney, the frontal cortex, and the cerebellar promordium. Signal from lung had decreased and become ubiquitous. At E17.5, signal was most apparent in the frontal cortex and cerebellar primordium of the brain, the snout, Meckel's cartilage, submandibular gland, spinal column, and capsule of the kidney which had the strongest signal. Signal was also seen from the smooth muscle of the gut. At E18.5, the pattern was nearly identical to that seen at E17.5. At P1.5, the pattern was very similar to that seen at E17.5 and 18.5 with strongest signal seen from Meckel's cartilage, basiocippital and basisphenoid bone, spinal column, developing cerebellum, and capsule of the kidney. By this stage of development, expression in most other tissues and organs had dropped to nearly background levels.

Human and mouse TANGO 128 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 77.8%. The human and mouse TANGO 128 full length cDNAs are 83.3% identical, as assessed using the same software and parameters as indicated (without the BLOSUM 62 scoring matrix). In the respective ORFs, calculated in the same fashion as the full length cDNAs, human and mouse TANGO 128 are 81.3% identical.

### Uses of TANGO 128 Nucleic Acids, Polypeptides, and Modulators Thereof

The TANGO 128 proteins of the invention bear some similarity to the VEGF family of growth factors. Accordingly, TANGO 128 proteins likely function in a similar manner as members of the VEGF family. Thus, TANGO 128 modulators can be used to treat any VEGF-associated disorders and modulate normal VEGF functions.

VEGF family members play a role in angiogenesis and endothelial cell growth. For example, VEGF is an endothelial cell specific mitogen and has been shown to be a potent angiogenic factor. Ferrara et al. (1992) Endocr. Rev. 13:18-32. Thus, several studies have reported that VEGF family members can serve as regulators of normal and pathological angiogenesis. Olofsson et al. (1996) Proc. Natl. Acad. Sci. USA 93:2576-2581; Berse et al. (1992) Mol. Biol. Cell. 3:211-220; Shweiki et al. (1992) Nature 359:843-845. Similarly, the TANGO 128 proteins of the invention likely play a role in angiogenesis. Accordingly, the TANGO 128 proteins, nucleic acids and/or modulators of the invention are useful angiogenic modulators. For example, the TANGO 128 proteins, nucleic acids and/or modulators can be used in the treatment of wounds, e.g., modulate wound healing, and/or the regrowth of vasculature, e.g., the regrowth of vasculature into ischemic organs, e.g., such as in coronary bypass. In addition, TANGO 128 proteins, nucleic acids and/or modulators can be used to promote growth of cells in culture for cell based therapies. Angiogenesis is also involved in pathological conditions including the growth and metastasis of tumors. In fact, tumor growth and metastasis have been shown to be dependent on the formation of new blood vessels. Accordingly, TANGO 128 polypeptides, nucleic acids and/or modulators thereof can be used to modulate angiogenesis in proliferative disorders such as cancer, (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilns' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, and retinoblastoma.

Because TANGO 128 is expressed in the reproductive tract, particularly in the ovaries and testis, the TANGO 128 polypeptides, nucleic acids and/or modulators thereof can be used to modulate the function, morphology, proliferation and/or differentiation of cells in the tissues in which it is expressed. For example, such molecules can be used to treat or modulate disorders associated with the testis including, without limitation, the Klinefelter syndrome (both the classic and mosaic forms), XX male syndrome, variococele, germinal cell aplasia (the Sertoli cell-only syndrome), idiopathic azoospermia or severe oligospermia, crpytochidism, and immotile cilia syndrome, or testicular cancer (primary germ cell tumors of the testis). In another example, TANGO 128 polypeptides, nucleic acids, or modulators thereof, can be used to treat testicular disorders, such as unilateral testicular enlargement (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

For example, the TANGO 128 polypeptides, nucleic acids and/or modulators thereof can be used modulate the function, morphology, proliferation and/or differentiation of the ovaries. For example, such molecules can be used to treat or modulate disorders associated with the ovaries, including, without limitation, ovarian tumors, McCune-Albright syndrome (polyostotic fibrous dysplasia). For example, the TANGO 128 polypeptides, nucleic acids and/or modulators can be used in the treatment of infertility.

The TANGO 128 polypeptides, nucleic acids and/or modulators thereof can be used to modulate the function, morphology, proliferation and/or differentiation of cells in the tissues of the reproductive tract other than the ovaries and testis. For example, such molecules can be used to treat or modulate disorders associated with the female reproductive tract including, without limitation, uterine disorders, e.g., hyperplasia of the endometrium, uterine cancers (e.g., uterine leiomyomoma, uterine cellular leiomyoma, leiomyosarcoma of the uterus, malignant mixed mullerian Tumor of uterus, uterine Sarcoma), and dysfunctional uterine bleeding (DUB).

### TANGO 140

In another aspect, the present invention is based on the discovery of cDNA molecules which encode a novel family of proteins referred to herein as TANGO 140 proteins. Described herein are TANGO 140-1, and TANGO 140-2 nucleic acid molecules and the corresponding polypeptides which the nucleic acid molecules encode.

For example, the tumor necrosis factor receptor (TNF-R) family to which the TANGO 140 proteins of the invention bear sequence similarity, are a family of cell surface proteins which function as receptors for cytokines and which contain conserved patterns of cysteine residues. Conserved cysteine residues, as used herein, refer to cysteine residues which are maintained within TANGO 140 family members (and/or TNF-R family members). This cysteine pattern is referred to herein as a tumor necrosis factor receptor (TNF-R) domain. These cysteine residues can form disulfide bonds which can affect the structural integrity of the protein. Thus, included within the scope of the invention are TANGO 140 proteins having at least one to four TNF-R domains, preferably two TNF-R domains. As used herein, a TNF-R domain refers to an amino acid sequence of about 25 to 50, preferably about 30 to 45, 30 to 40, and more preferably about 35 to 39 or 40 amino acids in length. A TNF-R domain of TANGO 140-1 extends, for example, from about amino acid II to amino acid 49 and/or from about amino acid 52 to amino acid 91; a TNF-R domain of TANGO 140-2 extends, for example, from about amino acid 25 to amino acid 63 and/or from about amino acid 66 to amino acid 105.

Conserved amino acid motifs, referred to herein as “consensus patterns” or “signature patterns”, can be used to identify TANGO 140 family members (and/or TNF-R family members) having a TNF-R domain. For example, the following signature pattern can be used to identify TANGO 140 family members: C-x (4, 6)-[FYH]-x (5, 10)-C-x (0, 2)-C-x (2, 3)-C-x (7, 11)-C-x (4, 6)-[DNEQSKP]-x (2)-C. The signature patterns or consensus patterns described herein are described according to Prosite Signature designation. Thus, all amino acids are indicated according to their universal single letter designation; “x” designates any amino acid; x(n) designates “n” number of amino acids, e.g., x (2) designates any two amino acids, e.g., x (4, 6) designates any four to six amino acids; and, amino acids in brackets indicates any one of the amino acids within the brackets, e.g., [FYH] indicates any of one of either F (phenylalanine), Y (tyrosine) or H (histidine). This consensus sequence can also be obtained as Prosite Accession Number PDOC00561. TANGO 140-1 has such a signature pattern at about amino acids 11 to 49 and at about amino acids 52 to 91 of SEQ ID NO:8. TANGO 140-2 has such a signature pattern at about amino acids 25 to 63 and at amino acids 66 to 105 of SEQ ID NO:10.

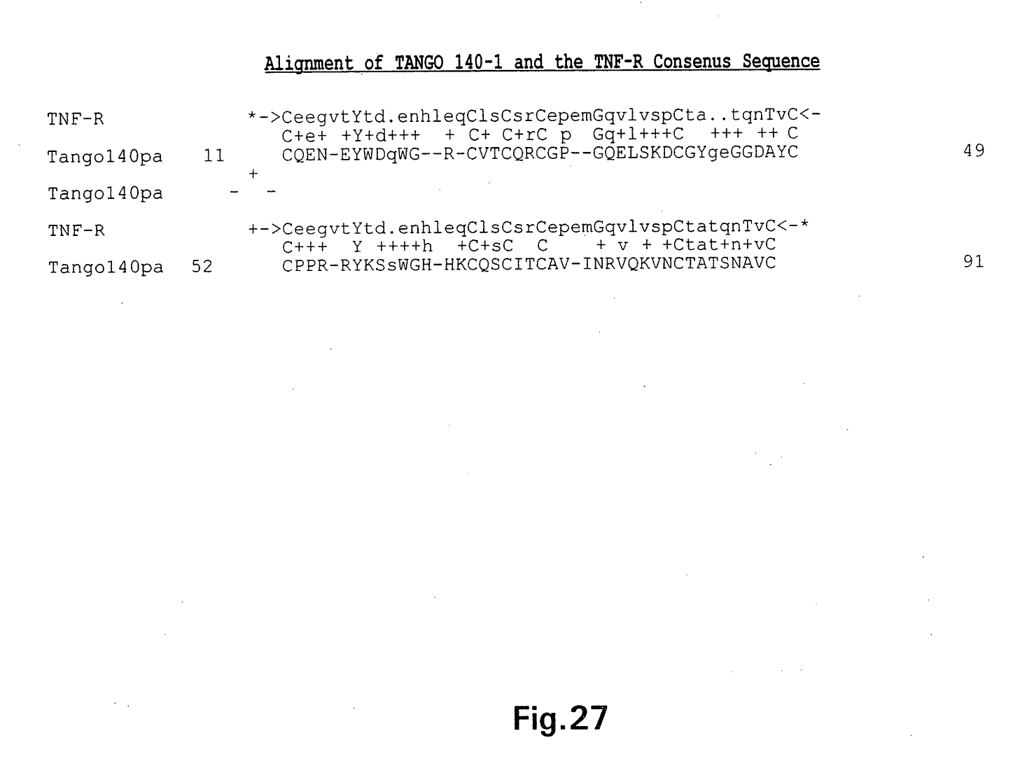

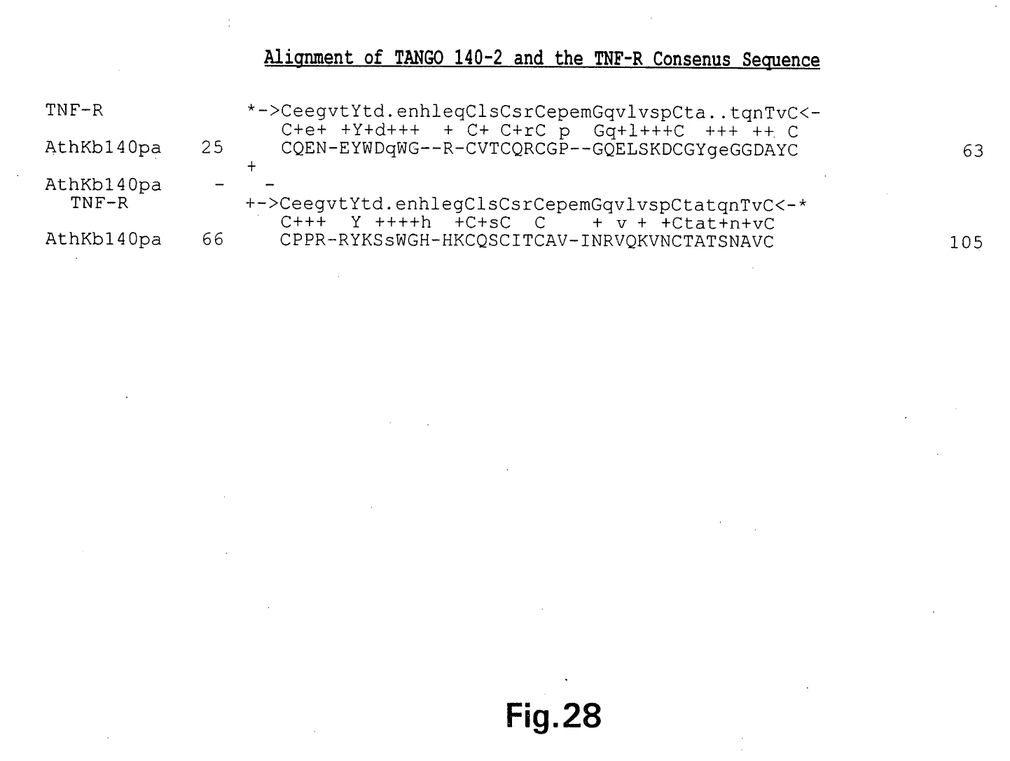

A TNF-R domain further contains at least about 2 to 10, preferably, 3 to 8, or 4 to 6 conserved cysteine residues. By alignment of a TANGO 140 family member with a TNF-R consensus sequence, conserved cysteine residues can be found. For example, as shown in FIG. 27, there is a first cysteine residue in the TNF-R consensus sequence that corresponds to a cysteine residue at amino acid 11 of the first TNF-R domain of TANGO 140-1; there is a second cysteine residue in the TNF-R consensus sequence that corresponds to a cysteine residue at amino acid 23 of the first TNF-R domain of TANGO 140-1; there is a third cysteine residue in the TNF-R consensus sequence that corresponds to a cysteine residue at amino acid 26 of the first TNF-R domain of TANGO 140-1; there is a fourth cysteine residue in the TNF-R consensus sequence that corresponds to a cysteine residue at amino acid 29 of the first TNF-R domain of TANGO 140-1; there is a fifth cysteine residue in the TNF-R consensus sequence that corresponds to a cysteine residue at amino acid 39 of the first TNF-R domain of TANGO 140-1; and/or there is a sixth cysteine residue in the TNF-R consensus sequence that corresponds to a cysteine residue at amino acid 49 of the first TNF-R domain of TANGO 140-1. In addition, conserved cysteine residues can be found at amino acids 52, 66, 69, 72, 83 and/or 91 of the second TNF-R domain of TANGO 140-1. Moreover, as shown in FIG. 28, conserved cysteine residues can be found at amino acids 25, 37, 40, 43, 53 and/or 63 of the first TNF-R domain of TANGO 140-2; and at amino acids 66, 80, 83, 86, 97 and/or 105 of TANGO-140-2. The TNF-R consensus sequence is available from the HMMer version 2.0 software as Accession Number PF00020. Software for HMM-based profiles is available from http://www.csc.ucsc.edu/research/compbio/sam.html and from http://genome.wustl.edu/eddy/hmmer.html.

The present invention also includes TANGO 140 proteins having a transmembrane domain. An example of a transmembrane domain includes from about amino acids 147 to 170 of TANGO 140-1.

Thus, in one embodiment, a TANGO 140 protein includes at least one TNF-R domain, preferably two, three or four TNF-R domains and is secreted. In another embodiment, a TANGO 140 protein of the invention includes at least one TNF-R domain, preferably two, three or four TNF-R domains, a transmembrane domain and is a membrane bound protein.

### Human TANGO 140-1

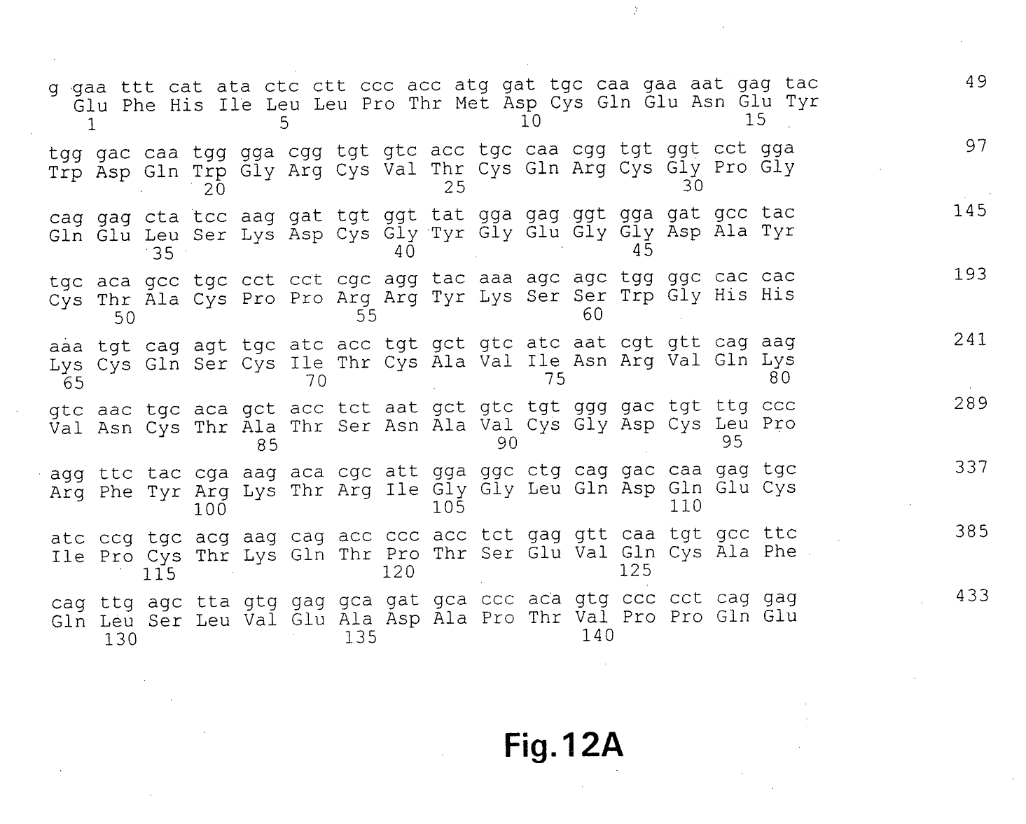

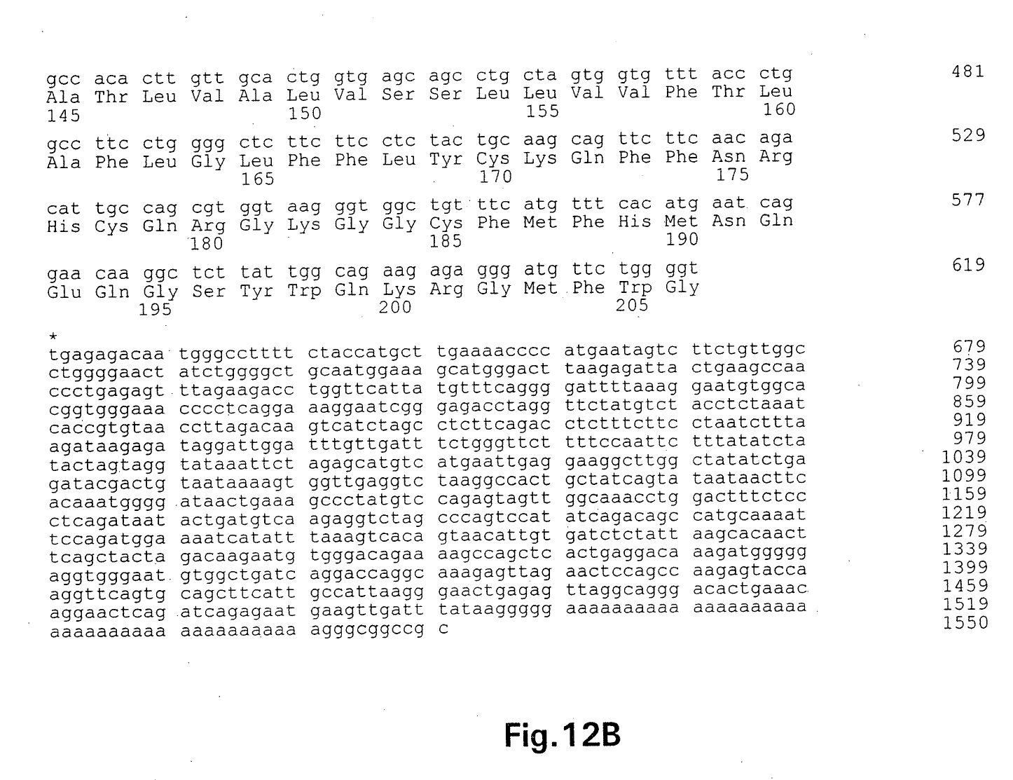

A cDNA encoding a portion of human TANGO 140-1 was identified by screening a stimulated human mesangial library. Human TANGO 140-1 includes a 1550 nucleotide cDNA (FIG. 12A-12B; SEQ ID NO:7). It is noted that the nucleotide sequence contains a Not I adapter sequence on the 3′ end. The open reading frame of TANGO 140-1 (nucleotides 2 to 619 of SEQ ID NO:7) encodes a 206 amino acid putative membrane protein (SEQ ID NO:8).

In one embodiment, human TANGO 140-1 includes an extracellular domain (about amino acids 1 to 146 of SEQ ID NO:8), a transmembrane (TM) domain (amino acids 147 to 170 of SEQ ID NO:8); and a cytoplasmic domain (amino acids 171 to 206 of SEQ ID NO:8). Alternatively, in another embodiment, a human TANGO 140-1 protein contains an extracellular domain at amino acid residues 1 to 146 of SEQ ID NO:8, a transmembrane domain at amino acid residues 147 to 170 of SEQ ID NO:8, and a cytoplasmic domain at amino acid residues 171 to 206 of SEQ ID NO:8.

The extracellular region of human TANGO 140-1 includes TNF-R domains from about amino acids 11 to 49 and from about amino acids 52-91 of SEQ ID NO:8.

A clone, EpDH137, which encodes human TANGO 140-1 was deposited as part of EpDHMix1 with the American Type Culture Collection, 10801 University Boulevard, Manassas, Va. 20110-2209) on Nov. 20, 1998 which was assigned Accession Number 98999. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

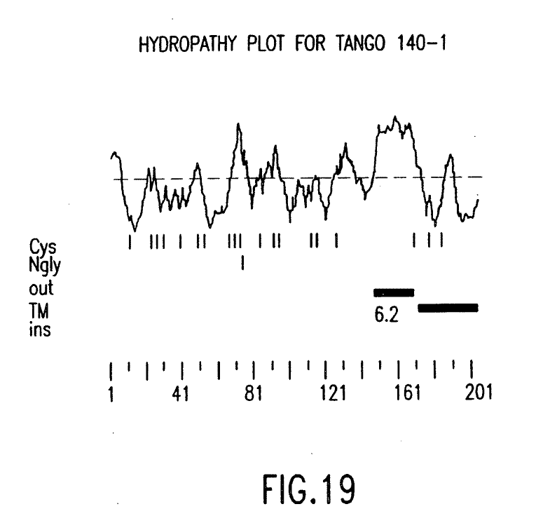

FIG. 19 depicts a hydropathy plot of human TANGO 140-1. As shown in the hydropathy plot, amino acids 147 to 170 of SEQ ID NO:8 correspond to a transmembrane domain of TANGO 140-1.

### Human TANGO 140-2

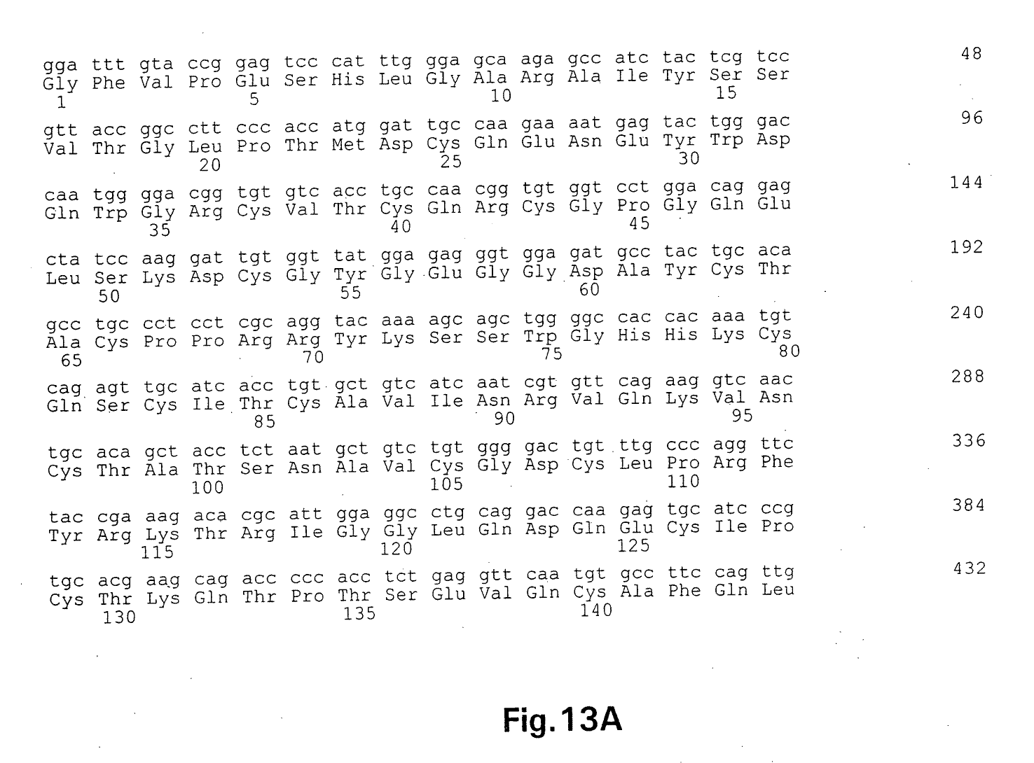

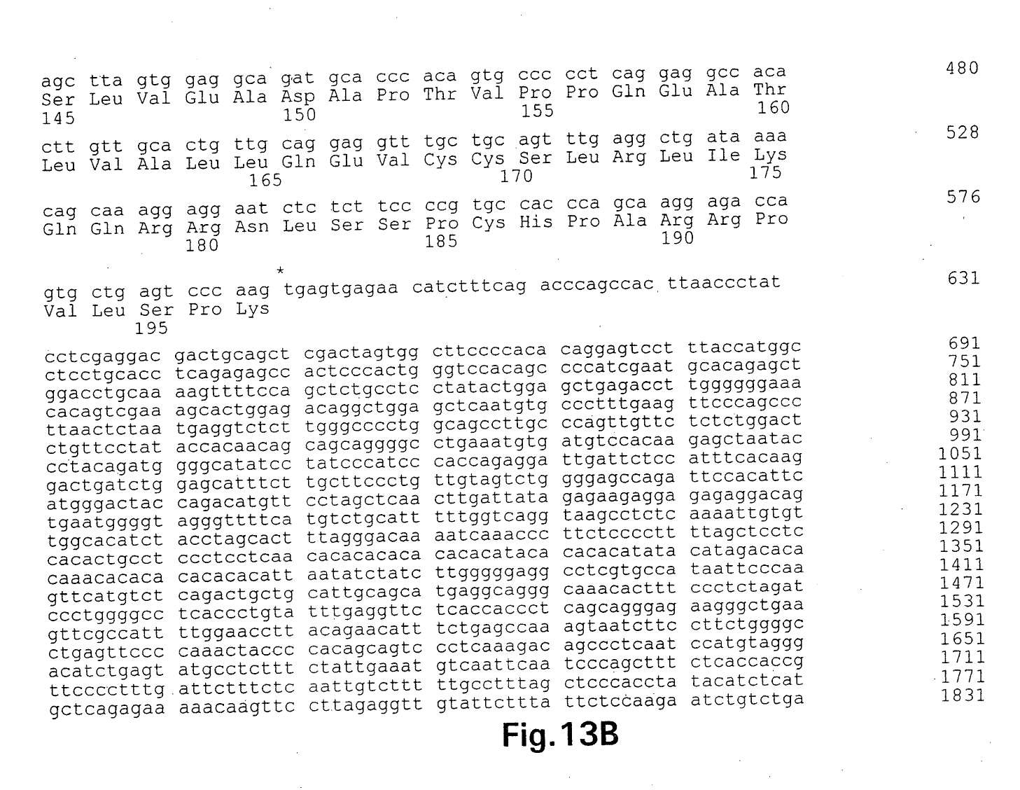

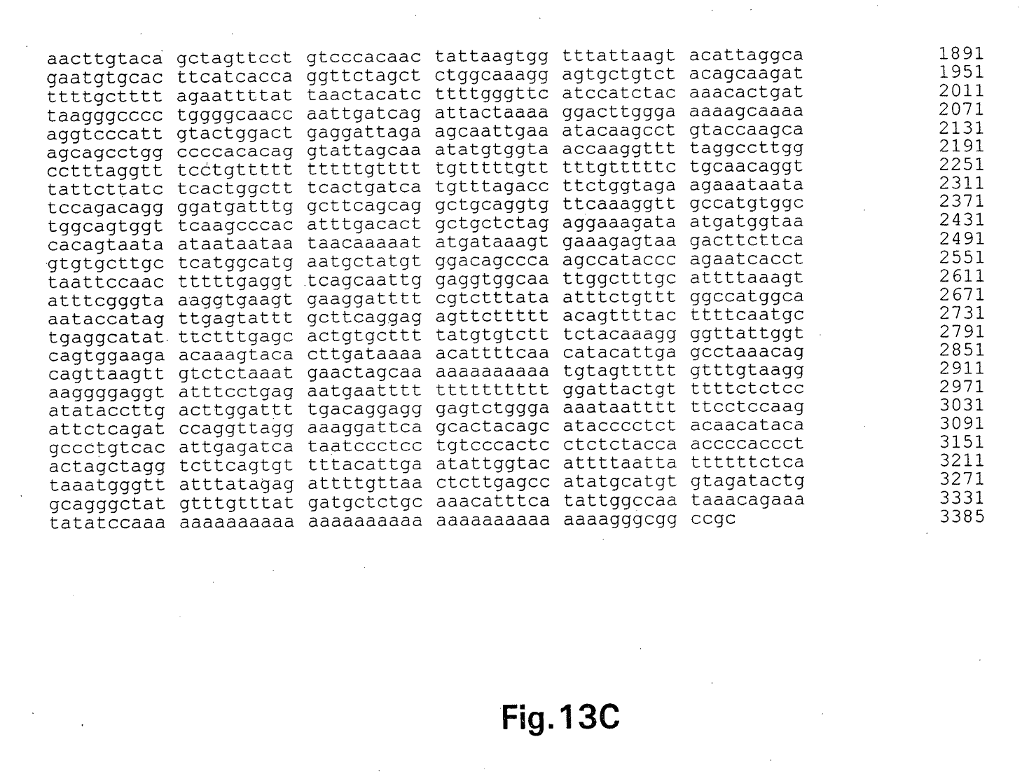

An additional clone having significant homology to human TANGO 140-1 was identified. The clone was sequenced and is likely to be a splice variant of TANGO 140-1. This variant is referred to herein as TANGO 140-2. The human TANGO 140-2 includes a 3385 nucleotide cDNA (FIG. 13A-13C; SEQ ID NO:9). It is noted that the nucleotide sequence contains a Not I adapter sequence on the 3′ end. The open reading frame of TANGO 140-2 (nucleotides 1 to 622 of SEQ ID NO:9) and encodes a 198 amino acid putative secreted protein (SEQ ID NO:10).

Human TANGO 140-2 also includes TNF-R domains from about amino acids 25 to 63, and from about amino acids 66 to 105.

TANGO 140-1 and TANGO 140-2 are identical from TANGO 140-1 amino acids 6 to 150 and TANGO 140-2 amino acids 20 to 164, yet differ at each of their respective amino and carboxy ends. These two genes are most likely splice variants of overlapping genetic material.

A clone, EpDH185, which encodes human TANGO 140-2 was deposited as part of EpDHMix1 with the American Type Culture Collection (10801 University Boulevard, Manassas, Va. 20110-2209) on Nov. 20, 1998 which was assigned Accession Number 98999. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

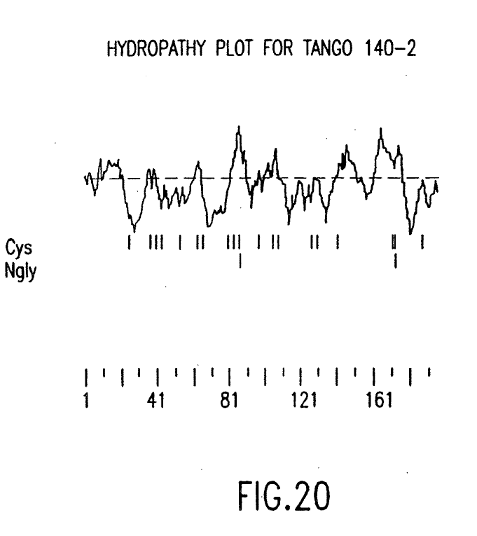

FIG. 20 depicts a hydropathy plot of TANGO 140-2.

### Uses of TANGO 140 Nucleic Acids, Polypeptides, and Modulators Thereof

The TANGO 140 proteins of the invention comprise a family of proteins having sequence similarity to members of the TNF-R superfamily. Thus, the TANGO 140 proteins of the invention are members of the TNF-R superfamily. Accordingly, TANGO 140 proteins likely function in a similar manner as members of the TNF-R family and TANGO 140 modulators can be used to treat any TNF-R/NGF-R-associated disorders.

For example, members of the tumor necrosis factor receptor (TNF-R) superfamily regulate a diverse range of cellular processes including cell proliferation, programmed cell death and immune responses. TNF-R family members are cell surface proteins which function as receptors for cytokines. Mallet et al. (1991) Immunology Today 12:220-223. For example, the binding of NGF to NGF-R causes neuronal differentiation and survival. Barde (1989) Neuron 2:1525-1534. Similarly, the TANGO 140 molecules of the invention can modulate neuronal differentiation and survival.

NGF (nerve growth factor) induces, inter alia, neurite outgrowth and promotes survival of embryonic sensory and sympathetic neurons. Nerve growth factor (NGF) is also involved in the development and maintenance of the nervous system. Thus, TANGO 140 polypeptides, nucleic acids and/or modulators thereof can be used to modulate the function, morphology, proliferation and/or differentiation of cells in the nervous system. Such molecules may be used in the treatment of neural disorders, including, without limitation, epilepsy, muscular dystrophy, and neurodegenerative diseases such as Alzheimer's disease, Parkinson's disease, and Huntington's disease).

In addition, both TGF-α and TGF-β bind to TGF-RI and TGF-RII, leading to a diverse range of effects including inflammation and tumor cell death. Beutler et al. (1989) Ann. Rev. Immunol. 7:625-655; Sprang (1990) Trends Biochem. Sci. 15:366-368. Thus, the TANGO 140 proteins of the invention are likely to bind directly or indirectly to a soluble protein, e.g., a cytokine, or membrane-bound protein, and play a role in modulating inflammation, cell proliferation, and/or apoptosis.

In light of the similarity of TANGO 140, TANGO 140 polypeptides, nucleic acids and/or modulators thereof can be used to treat TANGO 140 associated disorders which can include TNF-related disorders (e.g., acute myocarditis, myocardial infarction, congestive heart failure, T cell disorders (e.g., dermatitis, fibrosis)), immunological differentiative and apoptotic disorders (e.g., hyper-proliferative syndromes such as systemic lupus erythematosus (lupus)), and disorders related to angiogenesis (e.g., tumor formation and/or metastasis, cancer). Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

Moreover, as TANGO 140 is expressed in a stimulated mesangial library, the TANGO 140 polypeptides, nucleic acids and/or modulators thereof can be used to modulate the function, morphology, proliferation and/or differentiation of cells in the tissues in which it is expressed. Mesangial cells are known to play an important role in maintaining structure and function of the glomerulus and in the pathogenesis of glomerular diseases. Moreover, the local production of chemokines by mesangial cells has been linked to inflammatory processes within the glomerulus. Also, it is known that high glucose directly increases oxidative stress in glomerular mesangial cells, a target cell of diabetic nephropathy.

Thus, TANGO 140 polypeptides, nucleic acids and/or modulators thereof can be used to modulate the function, morphology, proliferation and/or differentiation of cells in the kidney. Such molecules can also be used to treat disorders associated with abnormal or aberrant metabolism or function of cells in the kidney. Therefore, such molecules can be used to treat or modulate renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcernic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

### TANGO 197

In one aspect, the present invention is based on the discovery of cDNA molecules which encode a novel family of proteins referred to herein as TANGO 197 proteins.

For example, the type A module superfamily, which includes proteins of the extracellular matrix and various proteins with adhesive function, have a von Willebrand factor type A (vWF) domain to which the TANGO 197 proteins of the invention bear similarity. This domain allows for the interaction between various cells and/or extracellular matrix (ECM) components. Thus, included within the scope of the invention are TANGO 197 proteins having a von Willebrand factor type A (vWF) domain. As used herein, a vWF domain refers to an amino acid sequence of about 150 to 200, preferably about 160 to 190, 170 to 180, and more preferably about 172 to 175 amino acids in length. A vWF domain of TANGO 197 extends, for example, from about amino acids 44 to 215.

Conserved amino acid motifs, referred to herein as “consensus patterns” or “signature patterns”, can be used to identify TANGO 197 family members having a vWF domain. For example, the following signature pattern can be used to identify TANGO 197 family members: D-x (2)-F-[ILV]-x-D-x-S-x (2, 3)-[ILV]-x (10, 12)-F. The signature patterns or consensus patterns described herein are described according to the following designation: all amino acids are indicated according to their universal single letter designation; “x” designates any amino acid; x(n) designates “n” number of amino acids, e.g., x (2) designates any two amino acids, e.g., x (2, 3) designates any of two to three amino acids; and, amino acids in brackets indicates any one of the amino acids within the brackets, e.g., [ILV] indicates any of one of either I (isoleucine), L (leucine) or V (valine). TANGO 197 has such a signature pattern at about amino acids 44 to 65.

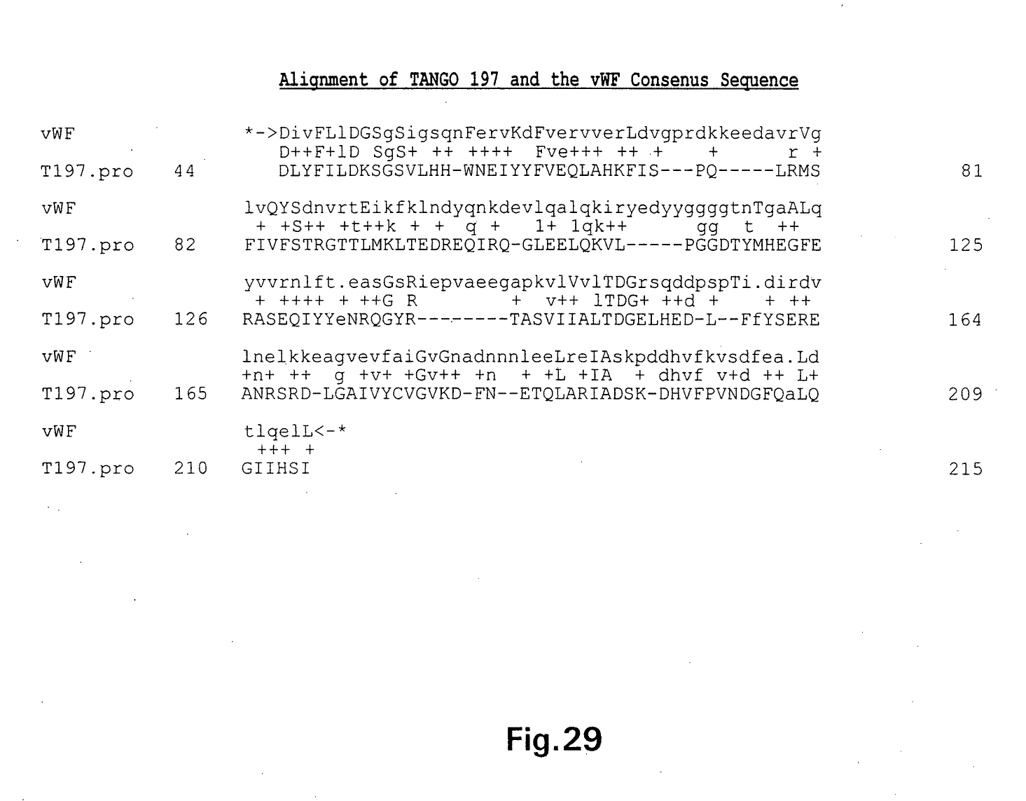

An alignment of TANGO 197 and the vWF consensus sequence is shown in FIG. 29. The vWF consensus sequence is available from the HMMer 2.0 software as Accession Number PF00092. Software for HMM-based profiles is available from http://www.csc.ucsc.edu/research/compbio/sam.html and from http://genome.wustl. edu/eddy/hmer.html.

Also included within the scope of the present invention are TANGO 197 proteins having a signal sequence.

In certain embodiments, a TANGO 197 family member has the amino acid sequence of SEQ ID NO:12, and the signal sequence is located at amino acids 1 to 25, 1 to 26, 1 to 27, 1 to 28, or 1 to 29. In such embodiments of the invention, the domains and the mature protein resulting from cleavage of such signal peptides are also included herein. Thus, in another embodiment, a TANGO 197 protein contains a signal sequence of about amino acids 1 to 27 which results in an extracellular domain consisting of amino acids' 28 to 301, and a mature TANGO 197 protein corresponding to amino acids 28 to 333 of SEQ ID NO: 12. The signal sequence is normally cleaved during processing of the mature protein.

### Human TANGO 197

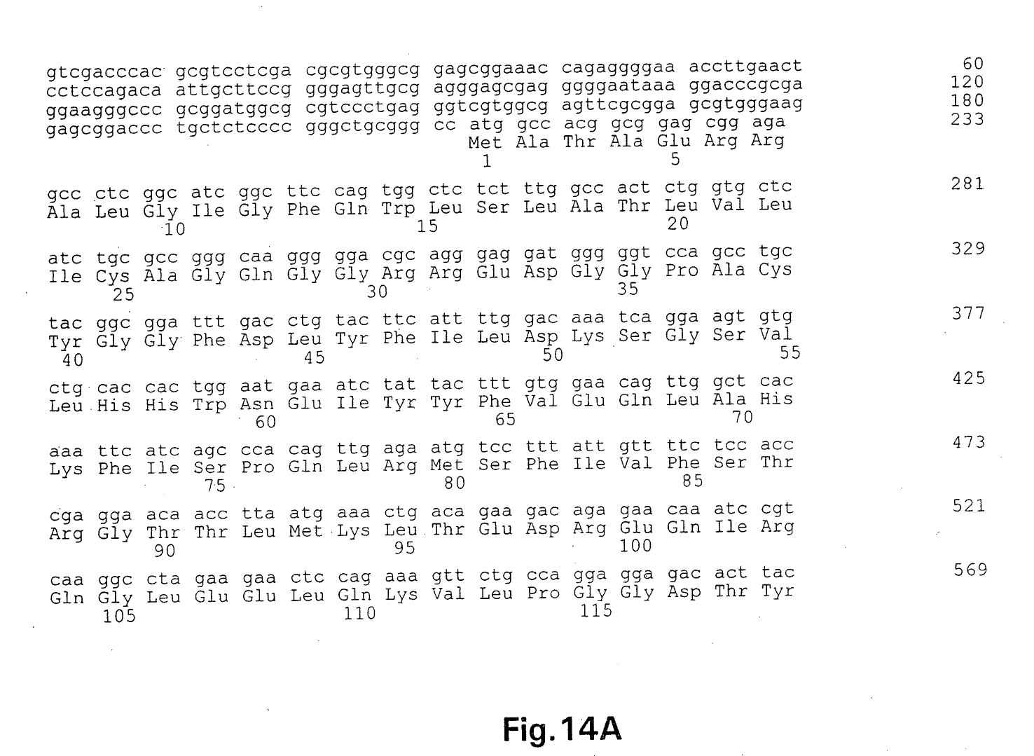

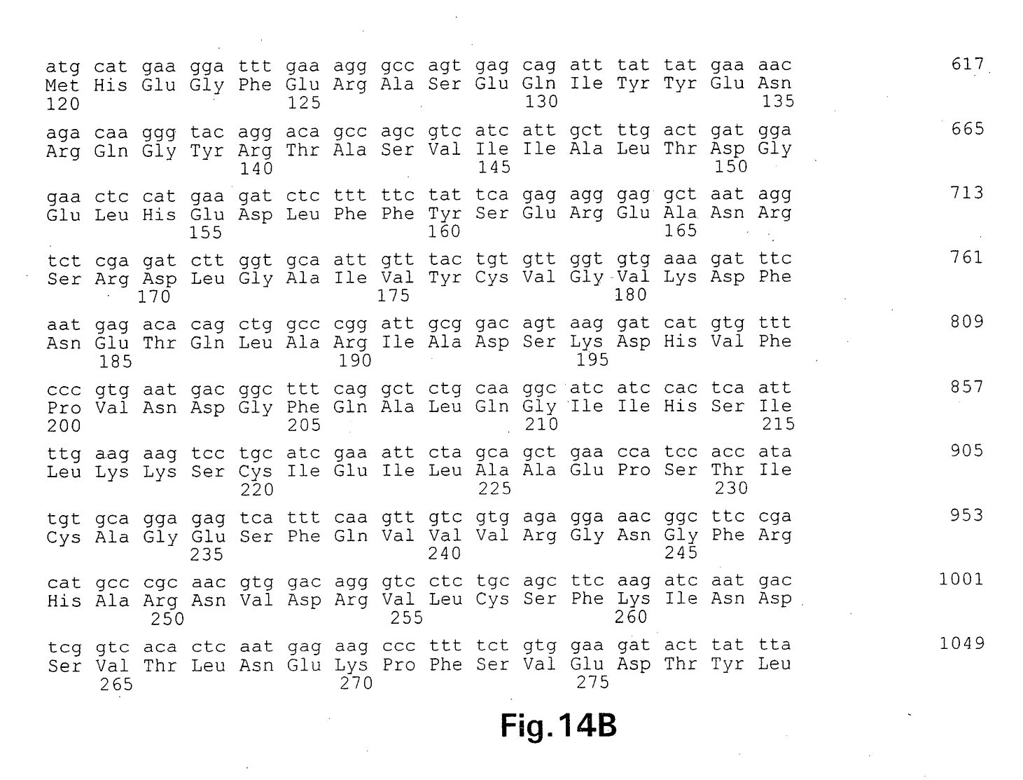

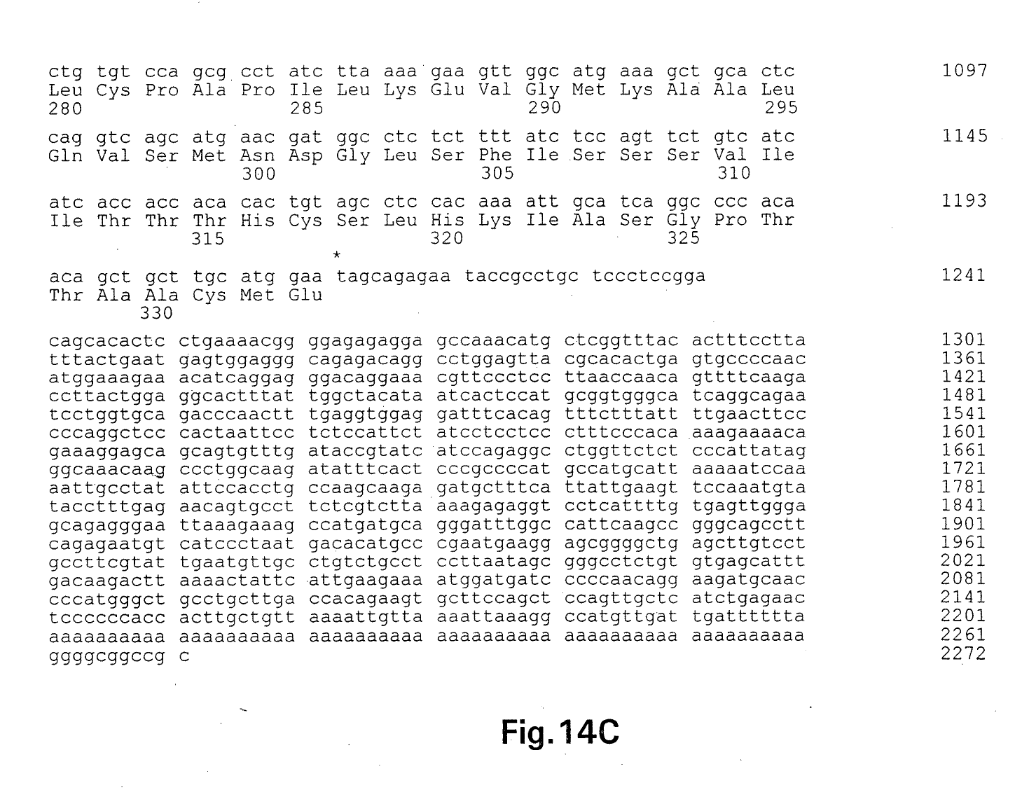

A cDNA encoding a portion of human TANGO 197 was identified by screening a human fetal lung library. An additional screen of an osteoclast library was performed to obtain a clone comprising a full length human TANGO 197. Human TANGO 197 includes a 2272 nucleotide cDNA (FIG. 14A-14C; SEQ ID NO:11). It is noted that the nucleotide sequence contains Sal I and Not I adapter sequences on the 5′ and 3′ ends, respectively. The open reading frame of this cDNA (nucleotides 213 to 1211 of SEQ ID NO:11) encodes a 333 amino acid transmembrane protein (SEQ ID NO: 12).

The signal peptide prediction program SIGNALP (Nielsen et al. (1997) Protein Engineering 10:1-6) predicted that human TANGO 197 includes a 27 amino acid signal peptide (amino acids I to about amino acid 27 of SEQ ID NO: 12) preceding the mature TANGO 197 protein (corresponding to about amino acid 28 to amino acid 333 of SEQ ID NO: 12).

Human TANGO 197 includes a vWF domain from about amino acids 44 to 215 of SEQ ID NO: 12.

A clone, EpDH213, which encodes human TANGO 197 was deposited as part of EpDHMix1 with the American Type Culture Collection (ATCC®, 10801 University Boulevard, Manassas, Va. 20110-2209) on Nov. 20, 1998 which was assigned Accession Number 98999. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

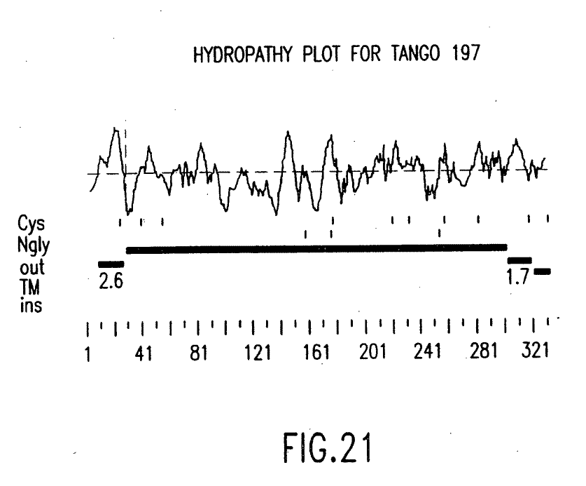

FIG. 21 depicts a hydropathy plot of human TANGO 197. As shown in the hydropathy plot, the hydrophobic region at the beginning of the plot which corresponds to about amino acids 1 to 27 is the signal sequence of TANGO 197.

In one embodiment, human TANGO 197 protein is a transmembrane protein that contains an extracellular domain at amino acid residues 28-301 of SEQ ID NO:12, a transmembrane domain at amino acid residues 302 to 319 of SEQ ID NO: 12, and a cytoplasmic domain at amino acid residues 320-333 of SEQ ID NO: 12. Alternatively, in another embodiment, a human TANGO 197 protein contains an extracellular domain at amino acid residues 320 to 333 of SEQ ID NO:12, a transmembrane domain at amino acid residues 302 to 319 of SEQ ID NO:12, and a cytoplasmic domain at amino acid residues 1 to 301 of SEQ ID NO: 12.

Northern analysis of human TANGO 197 mRNA expression revealed expression in a wide variety of tissues such as brain, skeletal muscle, colon, thymus, spleen, kidney, liver, and the small intestine. The highest levels of expression were seen in tissues such as the heart, placenta and lung. There was no expression of the transcript in peripheral blood leukocytes.

### Mouse TANGO 197

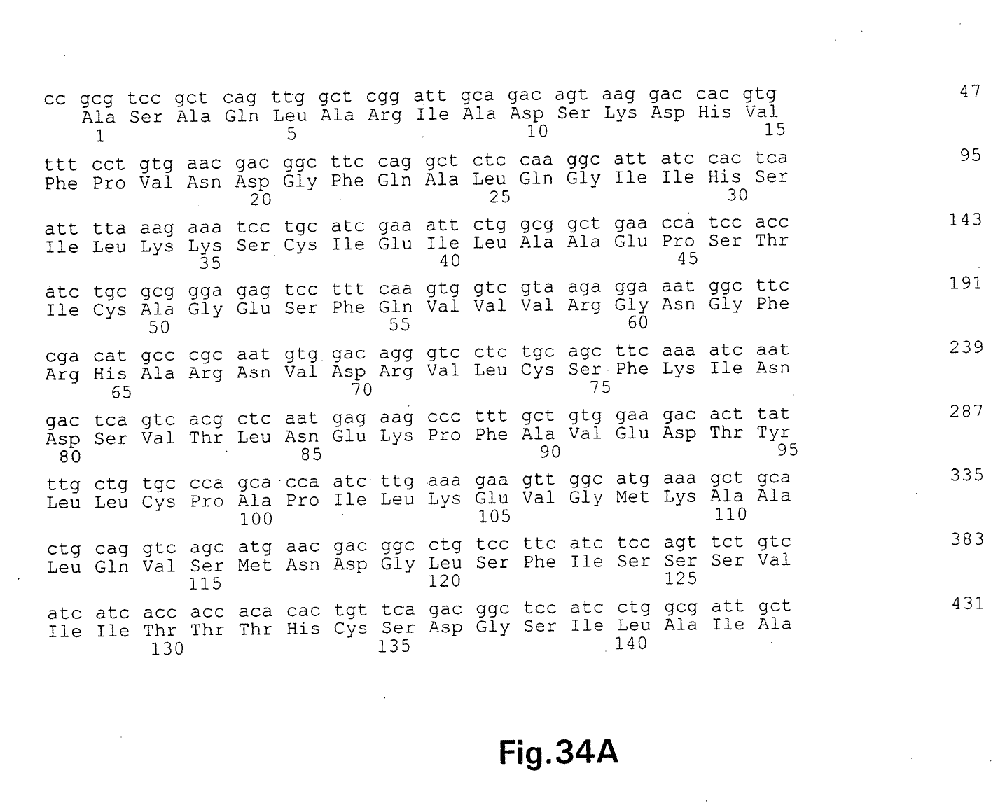

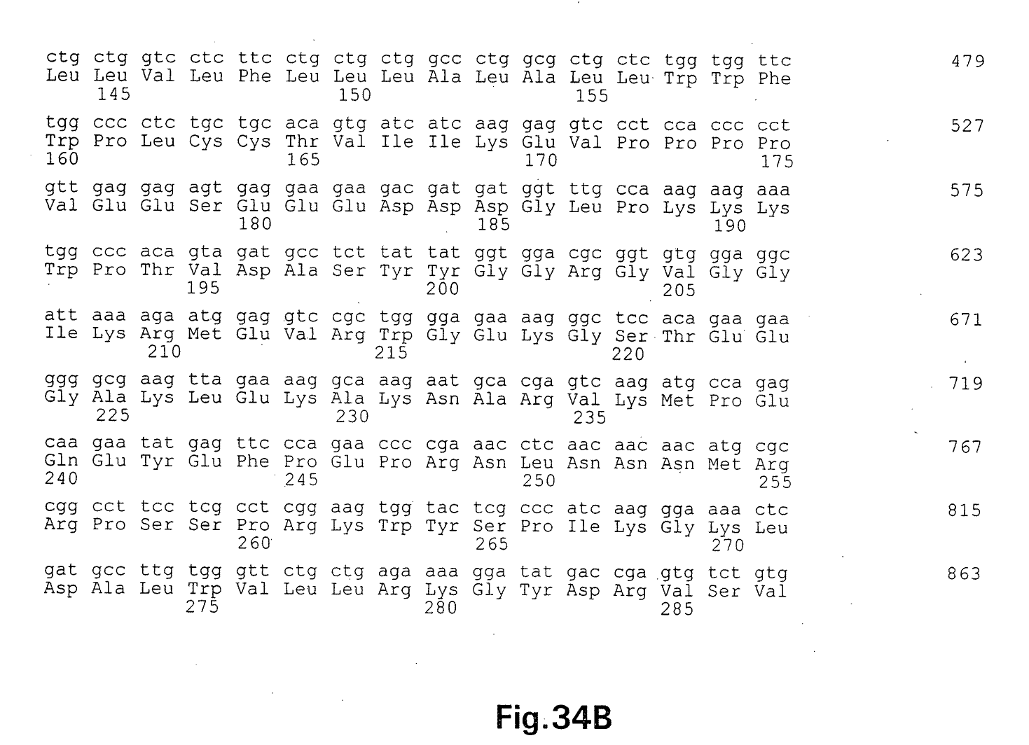

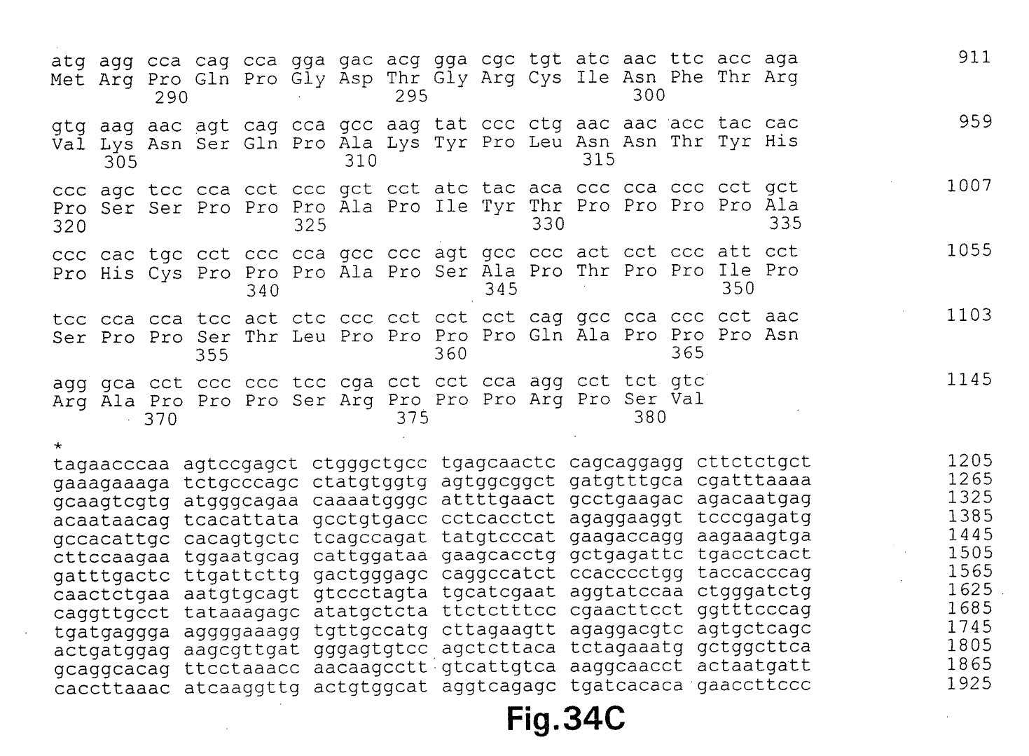

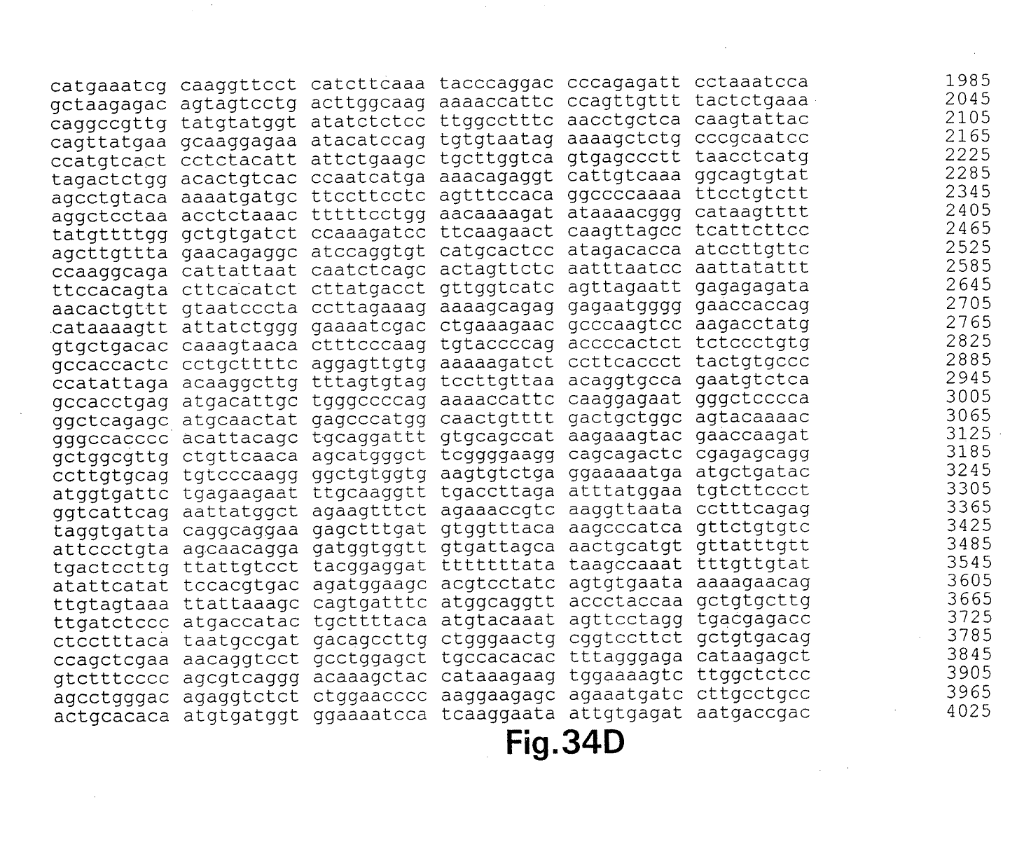

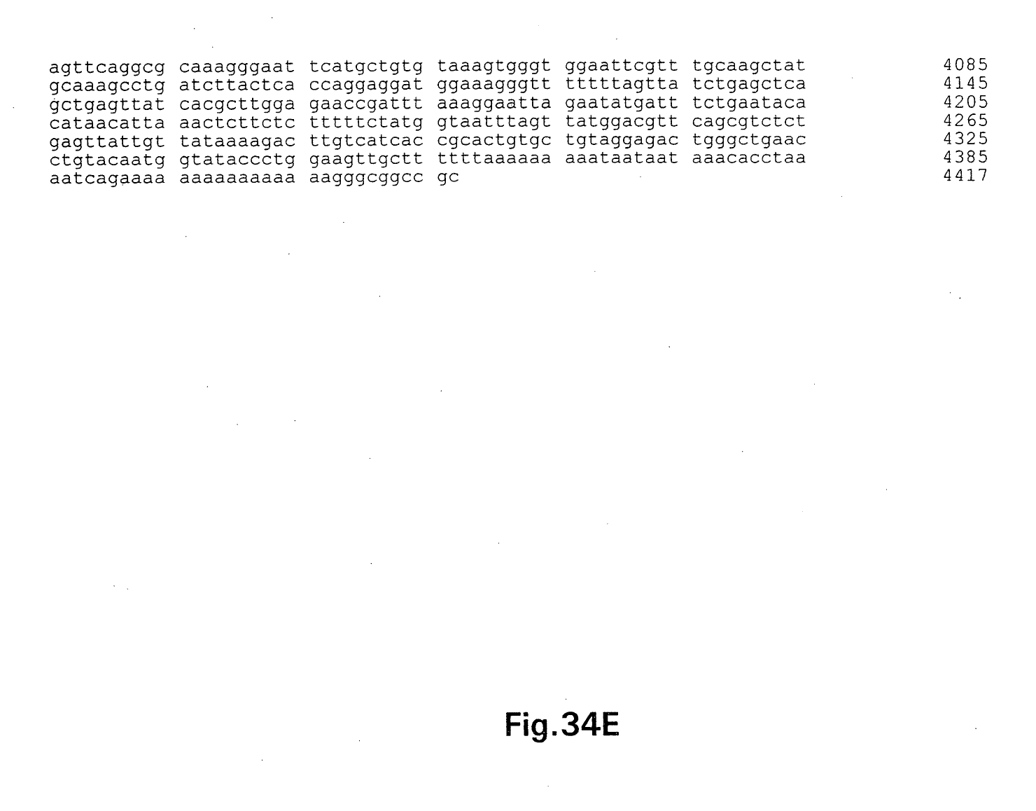

A mouse homolog of human TANGO 197 was identified. A cDNA encoding mouse TANGO 197 was identified by analyzing the sequences of clones present in a mouse testis (Sertoli TM4 cells) cDNA library. This analysis led to the identification of a clone, jtmzb062c08, encoding full-length mouse TANGO 197. The mouse TANGO 197 cDNA of this clone is 4417 nucleotides long (FIG. 34A-34D; SEQ ID NO:23). It is noted that the nucleotide sequence contains a Not Iadapter sequence on the 3′ end. The open reading frame of this cDNA (nucleotides 3-1145 of SEQ ID NO:23) encodes a 381 amino acid transmembrane protein (SEQ ID NO:24).

In one embodiment, mouse TANGO 197 protein is a transmembrane protein that contains an extracellular domain at amino acid residues 161 to 381 of SEQ ID NO:24, a transmembrane domain at amino acid residues 139 to 160 of SEQ ID NO:24, and a cytoplasmic domain at amino acid residues 1 to 138 of SEQ ID NO:24. Alternatively, in another embodiment, a mouse TANGO 197 protein contains an extracellular domain at amino acid residues 1 to 139 of SEQ ID NO:24, a transmembrane domain at amino acid residues139 to 160 of SEQ ID NO:24, and a cytoplasmic domain at amino acid residues 161 to 381 of SEQ ID NO:24.

Expression of mouse TANGO 197 mRNA was detected by a library array procedure. Briefly, the library array procedure entailed preparing a PCR mixture by adding to the standards reagents (Taq Polymerase, dNTPs, and PCR buffer) a vector primer, a primer internal to the gene of interest, and an aliquot of a library in which expression was to be tested. This procedure was performed with many libraries at a time in a 96 well PCR tray, with 80 or more wells containing libraries and a control well in which the above primers were combined with the clone of interest itself. The control well served as an indicator of the fragment size to be expected in the library wells, in the event the clone of interest was expressed within. Amplification was performed in a PCR machine, employing standard PCR conditions for denaturing, annealing, and elongation, and the resultant mixture was mixed with an appropriate loading dye and run on an ethidium bromide-stained agarose gel. The gel was later viewed with UV light after the DNA loaded within its lanes had time to migrate into the gels. Lanes in which a band corresponding with the control band was visible indicated the libraries in which the clone of interest was expressed.

Results of the library array procedure revealed strong expression in the choroid plexus, 12.5 day whole mouse embryo, LPS-stimulated osteoblast tissue, hyphae stimulated long term bone marrow cells. Weak expression was detected in TM4 (Sertoli cells), from testis, esophagus, LPS-stimulated osteoblast tissue. No expression was detected in differentiated 3T3, 10.5 day mouse fetus, mouse kidney fibrosis model, nephrotoxic serum (NTS), LPS-stimulated heart, LPS-stimulated osteoblasts, lung, mouse insulinoma (Nit-1), normal/hyperplastic islets (pancreas), normal spleen, 11.5 day mouse, LPS-stimulated lung, hypertropic heart, LPS-stimulated kidney, LPS-stimulated lymph node, mc/9 mast cells, 13.5 day mouse, LPS-stimulated anchored heart, normal thymus, Th2-ovarian-Tg, Balb C liver (bile duct ligation d2), normal heart, brain polysome (MPB), LPS-stimulated anchored liver, brain (EAE d10 model), th1-ovarian-Tg, heart, hypothalamus, lone term bone, marrow cells, megakaryocyte, LPS-stimulated spleen, hyphae-stimulated long term bone marrow, lung, angiogenic pancreatic islets, Th2, brain, LPS-stimulated thymus, LPS-stimulated microglial cells, testes (random-primed), tumor pancreatic islets, LPS-stimulated brain, LPS-stimulated alveolar macrophage cell line, mouse lung bleomycin model, pregnant uterus, and hypothalamus nuclei.

Human and mouse TANGO 197 sequences exhibit considerable similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−4), reveals a protein identity of 88.0%. The human and mouse TANGO 197 full length cDNAs are 52.8% identical, as assessed using the same software and parameters as indicated (without the BLOSUM 62 scoring matrix). In the respective ORFs, calculated in the same fashion as the full length cDNAs, human and mouse TANGO 197 are 51.6% identical.

### Uses of TANGO 197 Nucleic Acids, Polypeptides, and Modulators Thereof

As TANGO 197 exhibits expression in the lung, TANGO 197 polypeptides, nucleic acids, or modulators thereof, can be used to treat pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

Morever, as a species isoform of TANGO 197 was also isolated from a testis library, TANGO 197 polypeptides, nucleic acids, or modulators thereof, can be used to treat testicular disorders, examples of which are described elsewhere in this disclosure.

As discussed above, the vWF domain of TANGO 197 is involved in cellular adhesion and interaction with extracellular matrix (ECM) components. Proteins of the type A module superfamily which incorporate a vWF domain participate in multiple ECM and cell/ECM interactions. For example, proteins having a vWF domain have been found to play a role in cellular adhesion, migration, homing, pattern formation and/or signal transduction after interaction with several different ligands (Colombatti et al. (1993) Matrix 13:297-306).

Similarly, the TANGO 197 proteins of the invention likely play a role in various extracellular matrix interactions, e.g., matrix binding, and/or cellular adhesion. Thus, a TANGO 197 activity is at least one or more of the following activities: 1) regulation of extracellular matrix structuring; 2) modulation of cellular adhesion, either in vitro or in vivo; 3) regulation of cell trafficking and/or migration. Accordingly, the TANGO 197 proteins, nucleic acid molecules and/or modulators can be used to modulate cellular interactions such as cell-cell and/or cell-matrix interactions and thus, to treat disorders associated with abnormal cellular interactions.

TANGO 197 polypeptides, nucleic acids and/or modulators thereof can also be used to modulate cell adhesion in proliferative disorders, such as cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

### TANGO 212

In another aspect, the present invention is based on the discovery of cDNA molecules which encode a novel family of proteins referred to herein as TANGO 212 proteins.

For example, the EGF family to which the TANGO 212 proteins of the invention bear sequence similarity, are a family of mitogens which contain a conserved pattern of cysteine residues. Conserved cysteine residues, as used herein, refer to cysteine residues which are maintained within TANGO 212 family members (and/or EGF family members). This cysteine pattern is referred to herein as an epidermal growth factor (EGF) domain. These cysteine residues form disulfide bonds which can affect the structural integrity of the protein. Thus, included within the scope of the invention are TANGO 212 proteins having at least one, preferably two, three, four, or five EGF domain(s). As used herein, an EGF-domain refers to an amino acid sequence of about 25 to 50, preferably about 30 to 45, 30 to 40, and more preferably about 31, 35, 36 to 40 amino acids in length.

Conserved amino acid motifs, referred to herein as “consensus patterns” or “signature patterns”, can be used to identify TANGO 212 family members (and/or EGF family members) having an EGF domain. For example, the following signature pattern referred to herein as a EGF-like consensus sequence, can be used to identify TANGO 212 family members: C-x-C-x (5, 11)-G-x (2, 3)-C. TANGO 212 has such a signature pattern at about amino acids 80 to 91, amino acids 156 to 172, amino acids 200 to 217 and/or amino acids 245 to 258. An EGF domain of TANGO 212 extends, for example, from about amino acids 61 to 91, from about amino acids 98 to 132, from about amino acids 138 to 172, from about amino acids 178 to 217, and/or from about amino acids 223 to 258 of SEQ ID NO:14.

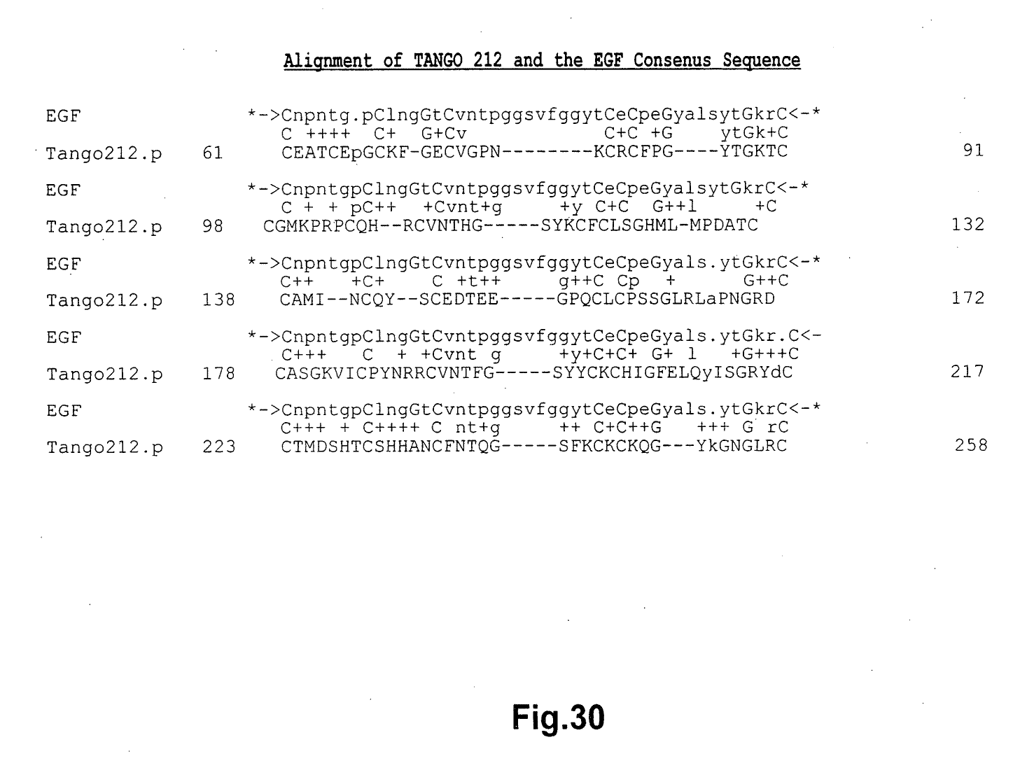

An EGF domain further contains at least about 2 to 10, preferably, 3 to 9, 4 to 8, or 6 to 7 conserved cysteine residues. By alignment of a TANGO 212 family member with an EGF-like consensus sequence, conserved cysteine residues can be found. For example, as shown in FIG. 30, there is a first cysteine residue in the EGF-like consensus sequence that corresponds to a cysteine residue at amino acid 61 of the first EGF domain of TANGO 212; there is a second cysteine residue in the EGF-like consensus sequence that corresponds to a cysteine residue at amino acid 69 of the first EGF domain of TANGO 212; there is a third cysteine residue in the EGF-like consensus sequence that corresponds to a cysteine residue at amino acid 74 of the first EGF domain of TANGO 212; there is a fourth cysteine residue in the EGF-like consensus sequence that corresponds to a cysteine residue at amino acid 80 of the first EGF domain of TANGO 212; there is a fifth cysteine residue in the EGF-like consensus sequence that corresponds to a cysteine residue at amino acid 82 of the first EGF domain of TANGO 212; and/or there is a sixth cysteine residue in the EGF-like consensus sequence that corresponds to a cysteine residue at amino acid 91 of the first EGF-domain of TANGO 212. In addition, conserved cysteine residues can be found at amino acids 98, 105, 109, 118, 120 and/or 132 of the second EGF domain of TANGO 212; at amino acids 138, 143, 147, 156, 158 and/or 172 of the third EGF domain of TANGO 212; at amino acids 178, 185, 191, 200, 202 and/or 217 of the fourth EGF domain of TANGO 212; and at amino acids 223, 230, 236, 245, 247 and/or 258 of the fifth EGF domain of TANGO 212 (SEQ ID NO:14). The EGF-like consensus sequence is available from the HMMer version 2.0 software as Accession Number PF00008. Software for HM-based profiles is available from http://www.csc.ucsc.edu/research/compbio/sam.html and from http://genome.wustl.edu/eddy/hmmer.html.

The present invention also features TANGO 212 proteins having a MAM domain. The MAM domain is associated with various adhesive proteins and as such is likely to have adhesive function. Within MAM domains are conserved cysteine residues which play a role in the adhesion of a MAM domain to other proteins. As used herein, a MAM domain refers to an amino acid sequence of about 120 to about 170, preferably about 130 to 160, 140 to 150, and more preferably about 145 to 147 amino acids in length.

Conserved amino acid motifs, referred to herein as “consensus patterns” or “signature patterns”, can be used to identify TANGO 212 family members having a MAM domain. For example, the following signature pattern can be used to identify TANGO 212 family members: G-x-[LIVMFY](2)-x (3)-[STA]-x (10, 11)-[LV]-x (4,6)-[LIVMF]-x (6, 7)-C-[LIVM]-x (3)-[LIVMFY]-x (3, 4)-[GSC]. The signature patterns or consensus patterns described herein are described according to the following designations: all amino acids are indicated according to their universal single letter designation; “x” designates any amino acid; x(n) designates “n” number of amino acids, e.g., x (2) designates any two amino acids, e.g., x (6, 7) designates any six to seven amino acids; and, amino acids in brackets indicates any one of the amino acids within the brackets, e.g., [STA] indicates any of one of either S (serine), T (threonine) or A (alanine). TANGO 212 has such a signature pattern at about amino acids 431 to 472.

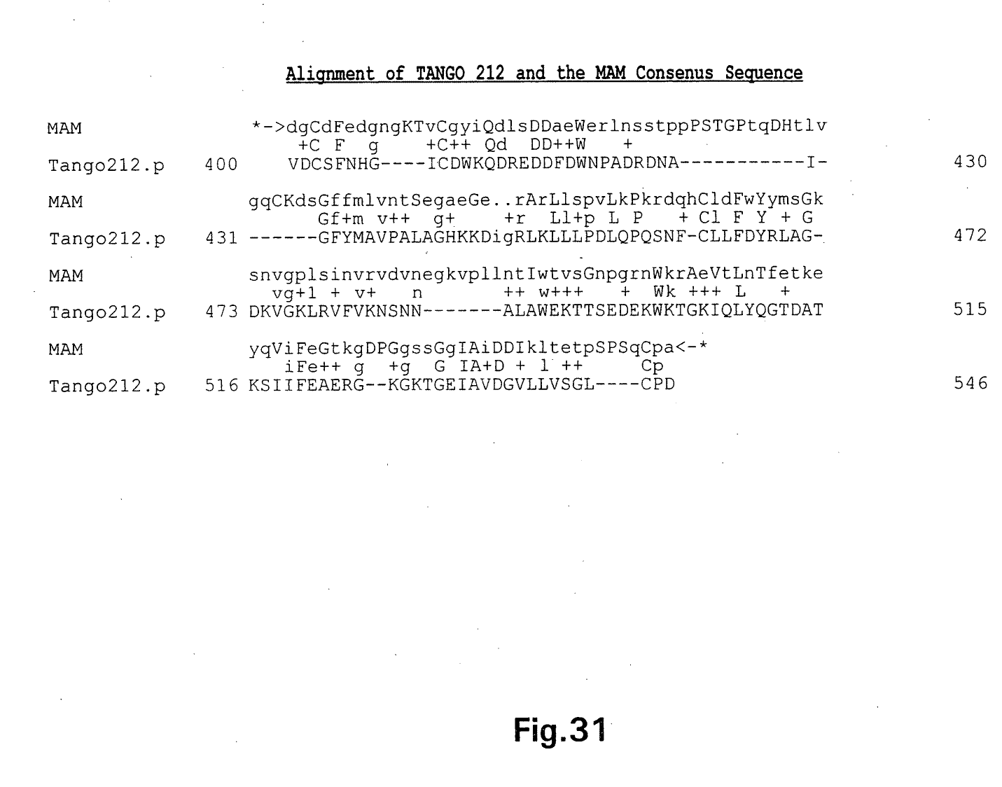

A MAM domain further contains at least about 2 to 6, preferably, 3 to 5, more preferably 4 conserved cysteine residues. By alignment of a TANGO 212 family member with a MAM consensus sequence, conserved cysteine residues can be found. For example, as shown in FIG. 31, there is a first cysteine residue in the MAM consensus sequence that corresponds to a cysteine residue at amino acid 402 of TANGO 212; there is a second cysteine residue in the MAM consensus sequence that corresponds to a cysteine residue at amino acid 409 of TANGO 212; there is a third cysteine residue in the MAM consensus sequence that corresponds to a cysteine residue at amino acid 463 of TANGO 212; and/or there is a fourth cysteine residue in the MAM consensus sequence that corresponds to a cysteine residue at amino acid 544 of TANGO 212 (SEQ ID NO:14). The MAM consensus sequence is available from the HMMer version 2.0 software as Accession Number PF00629. Software for HMM-based profiles is available from http://www.csc.ucsc.edu/research/compbio/sam.html and from http://genome.wustl.edu/eddy/hmmer.html.

Also included within the scope of the present invention are TANGO 212 proteins having a signal sequence.

In certain embodiments, a TANGO 212 family member has the amino acid sequence of SEQ ID NO:14, and the signal sequence is located at amino acids 1 to 16, 1 to 17, 1 to 18, 1 to 19, or 1 to 20. In such embodiments of the invention, the domains and the mature protein resulting from cleavage of such signal peptides are also included herein. For example, the cleavage of a signal sequence consisting of amino acids 1 to 18 results in a mature TANGO 212 protein corresponding to amino acids 19 to 553 of SEQ ID NO:14. The signal sequence is normally cleaved during processing of the mature protein.

In one embodiment, a TANGO 212 protein of the invention includes at least one EGF domain, preferably two, three, four, or five EGF domains and a MAM domain. In another embodiment, a TANGO 212 protein of the invention includes at least one EGF domain, preferably two, three, four, or five EGF domains, a MAM domain, a signal sequence, and is secreted.

### Human TANGO 212

A cDNA encoding human TANGO 212 was identified by screening a human fetal lung library. A clone, comprising TANGO 212, was selected for complete sequencing based on its ability to direct the secretion of a protein of approximately 30 kDa in ³⁵S labeled supernatants of 293T cells.

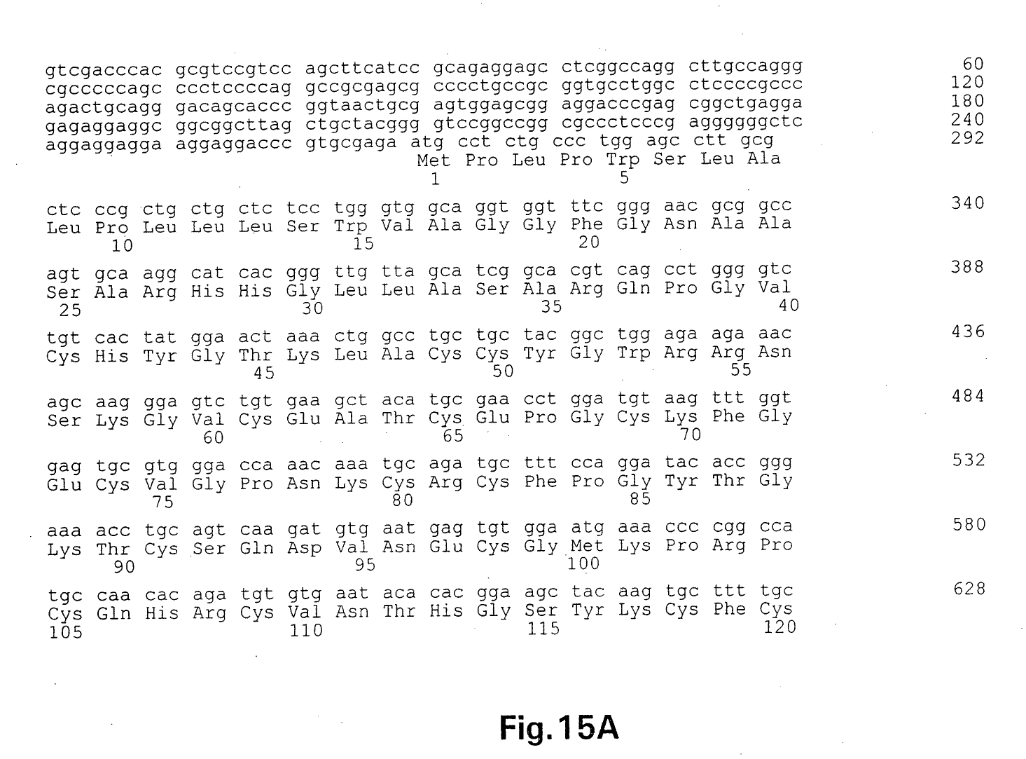

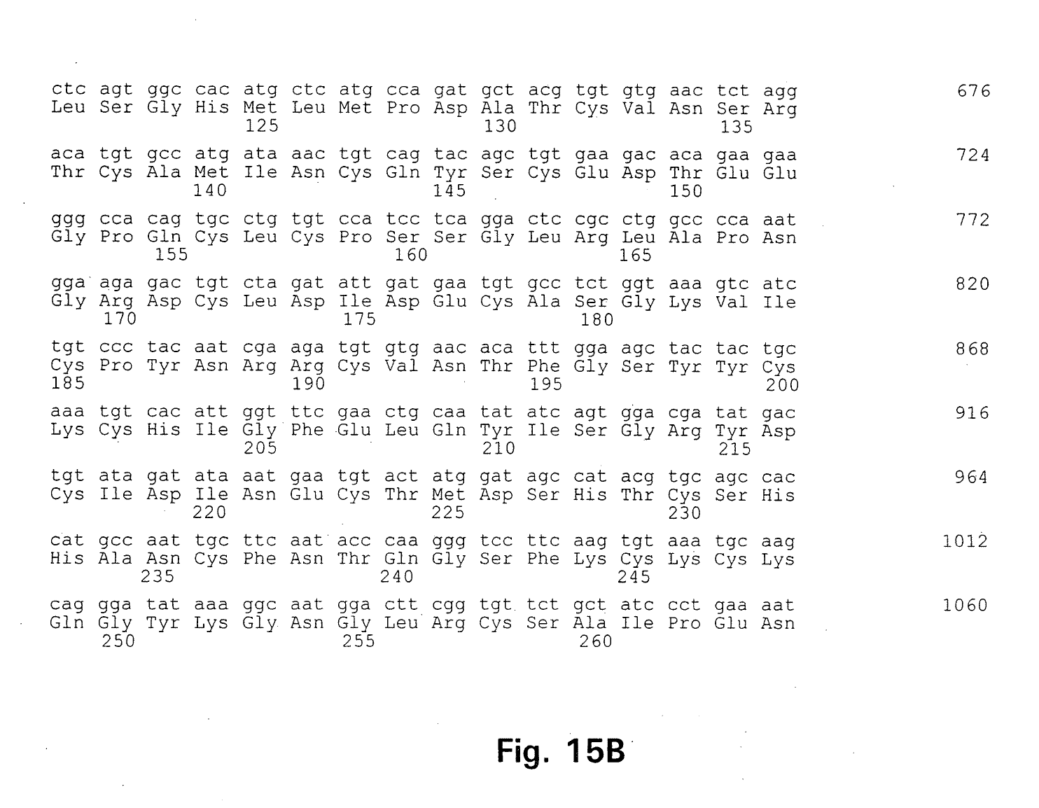

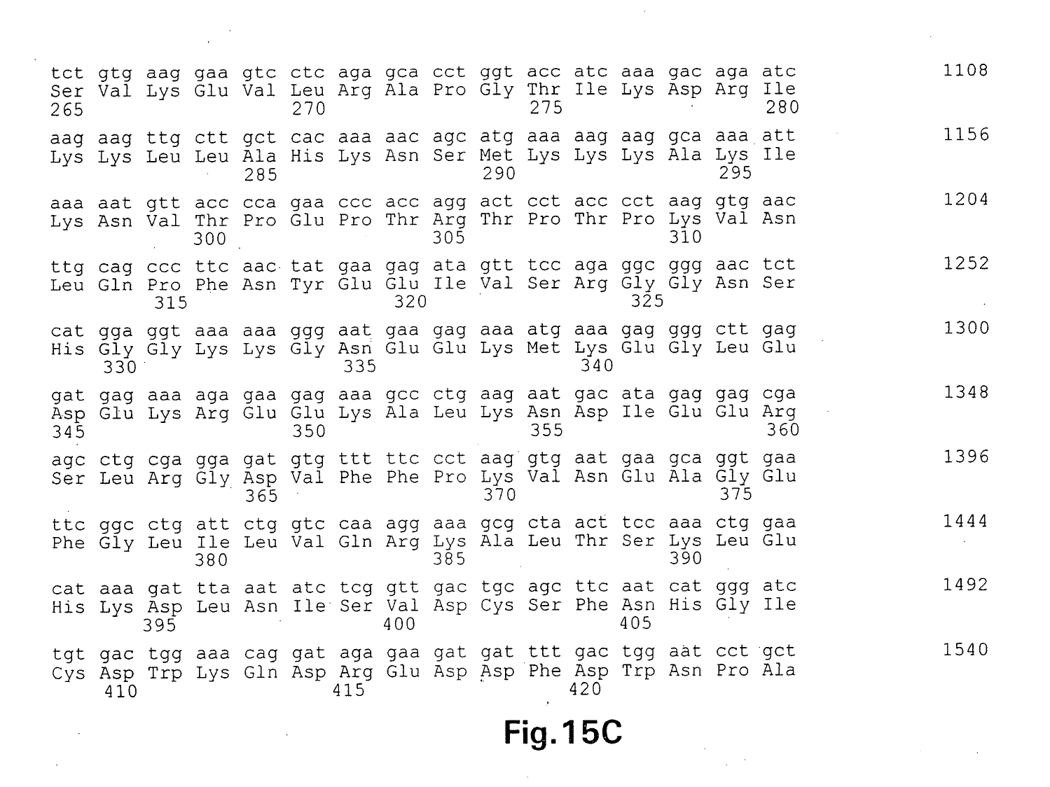

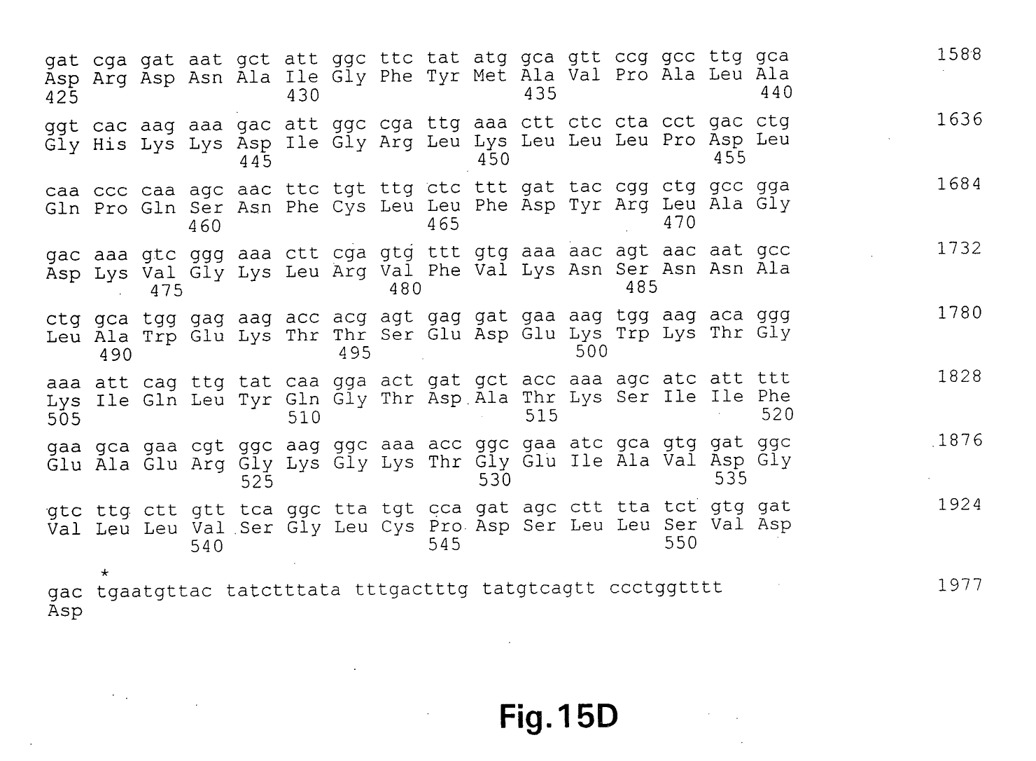

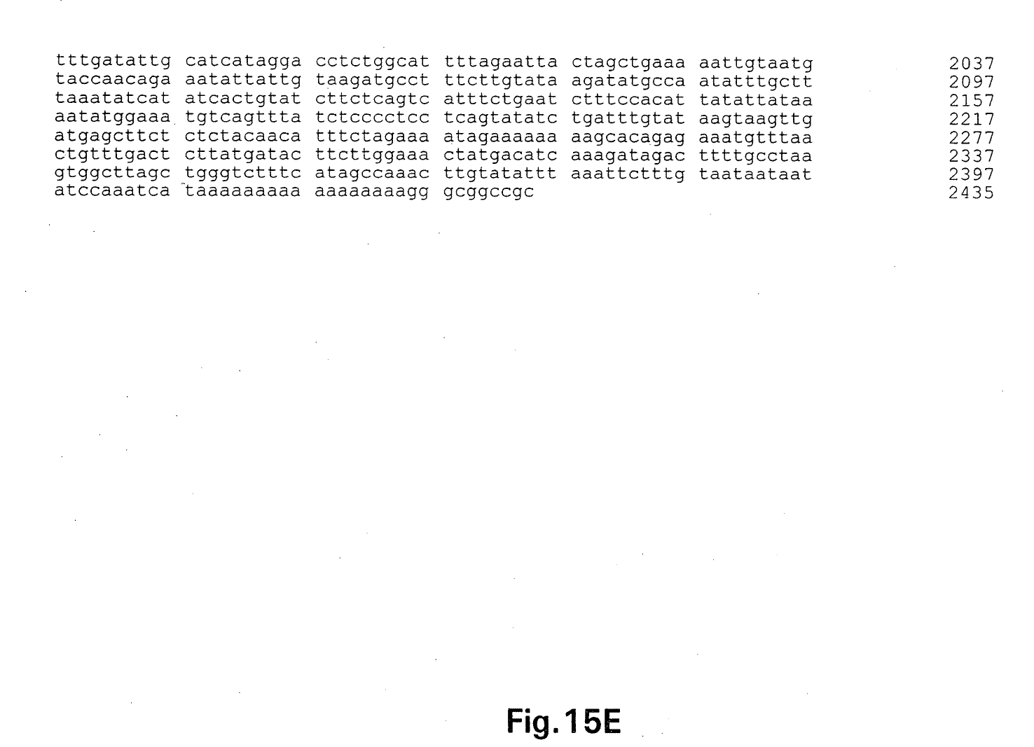

TANGO 212 includes a 2435 nucleotide cDNA (FIG. 15A-15E; SEQ ID NO: 13). It is noted that the nucleotide sequence contains Sal I and Not I adapter sequences on the 5′ and 3′ ends, respectively. The open reading frame of this cDNA (nucleotides 269 to 1927 of SEQ ID NO:13) encodes a 553 amino acid secreted protein (SEQ ID NO:14).

The signal peptide prediction program SIGNALP (Nielsen et al. (1997) Protein Engineering 10:1-6) predicted that human TANGO 212 includes an 18 amino acid signal peptide (amino acids 1 to about amino acid 18 of SEQ ID NO:14) preceding the mature TANGO 212 protein (corresponding to about amino acid 19 to amino acid 553 of SEQ ID NO: 14). Human TANGO 212 is predicted to have a molecular weight of approximately 61 kDa prior to cleavage of its signal peptide and a molecular weight of approximately 59 kDa subsequent to cleavage of its signal peptide. In addition, gel analysis of ³⁵S labeled supernatants of 293T cells transfected with TANGO 212 expression plasmid identified a band at approximately 30 kDa. Thus, further processing of human TANGO 212 is likely to occur.

Secretion of TANGO 212 was detected by transfection using SPOT analysis (SignalP Optimized Tool, or “SPOT”). Briefly, SPOT based analysis was performed using software (termed developed to identify signal peptide encoding RNAs, all forward orientation open reading frames in the DNA sequences and phrap (see http://bozeman.mbt.washington.edu/phrap.docs/phrap.html) pre-assembled DNA sequences from the library, starting with ATG and continuing for at least 19 non-stop codons, were translated. Signal peptides in the translated sequences were then predicted using the computer algorithm SignalP (Nielsen, H. et al.(1997) Protein Engineering 10:1-6), and those sequences scoring YES were saved. Open reading frames containing signal peptides with fewer than 20 amino acids after the predicted cleavage site were discarded. The translated sequences scoring YES in the SignalP analysis were then compared against a non-redundant protein database using BLAST 1.4, PAM10 matrix with score cut-offs (parameters S and S2) set to 150. Translated sequences with a match under these conditions were discarded.

Human TANGO 212 includes five EGF domains from about amino acids 61 to 91, amino acids 98 to 132, amino acids 138 to 172, amino acids 178 to 217, and amino acids 223 to 258. Human TANGO 212 further includes a MAM domain (about amino acids 400 to 546).

A clone, EpDH202, which encodes human TANGO 212 was deposited with the American Type Culture Collection (ATCC®, 10801 University Boulevard, Manassas, Va. 20110-2209) on Sep. 10, 1998 and assigned Accession Number 202171. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

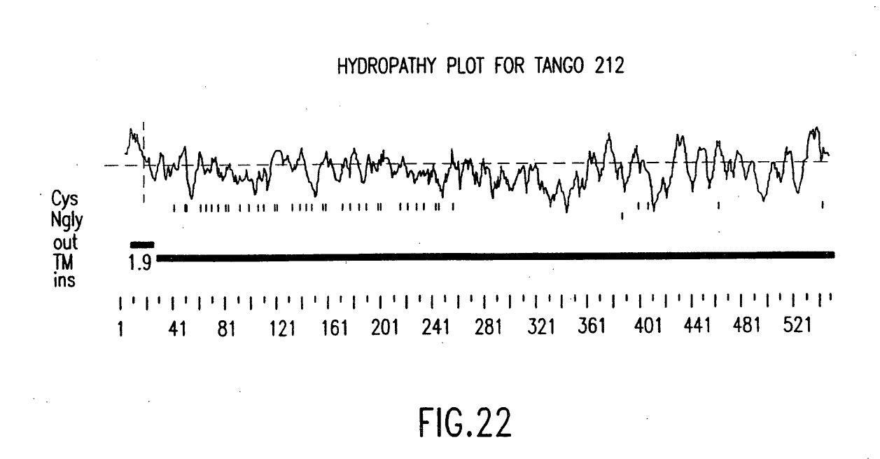

FIG. 22 depicts a hydropathy plot of human TANGO 212. As shown in the hydropathy plot, the hydrophobic region at the beginning of the plot which corresponds to about amino acids 1 to 18 is the signal sequence of TANGO 212, cleavage of which yields the mature protein of amino acids 19 to 553.

Northern analysis of human TANGO 212 mRNA expression revealed that is expressed at a very high level in placenta, strong levels in fetal lung and kidney, and at a low level in adult lung. No expression was seen in adult heart, liver, brain, skeletal muscle, kidney, pancreas, spleen, thymus, prostate, testis, ovary, small intestine, colon, peripheral blood leukocytes, or fetal brain and liver.

### Mouse TANGO 212

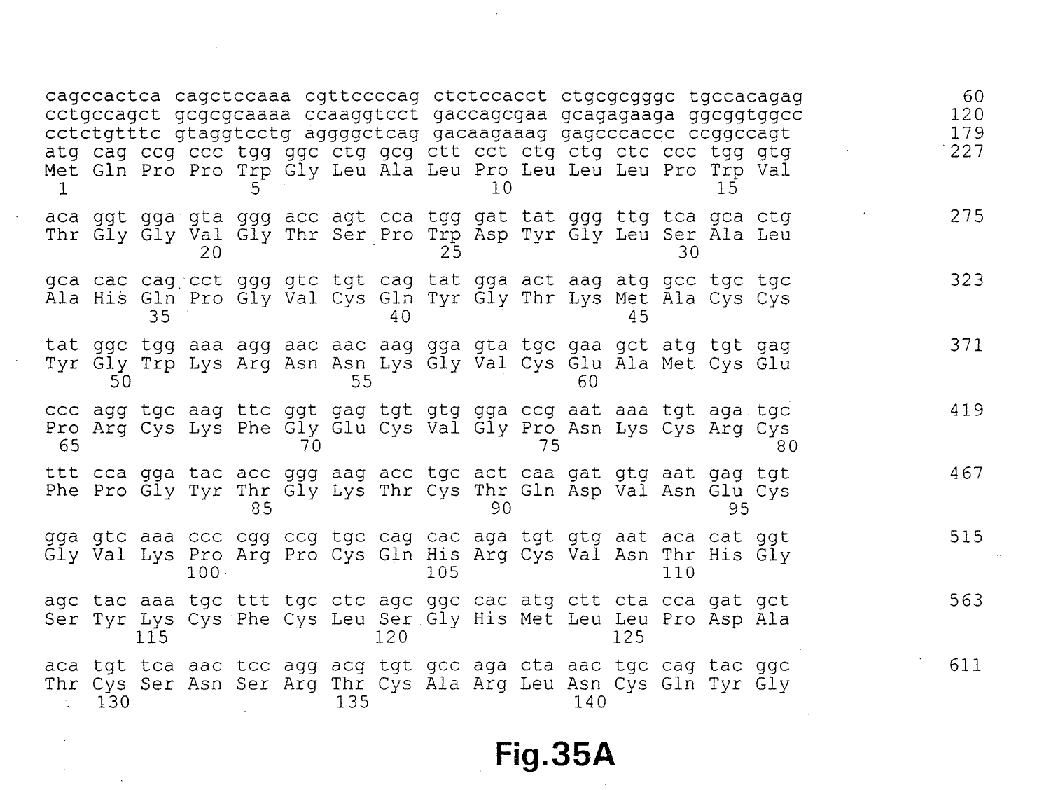

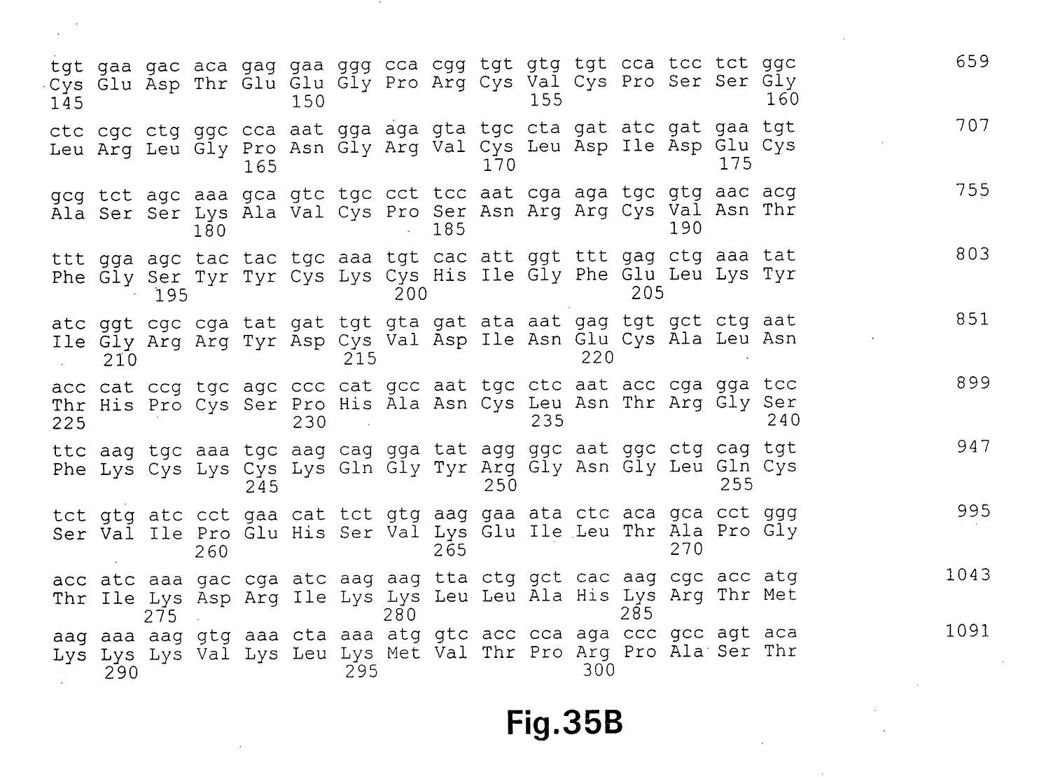

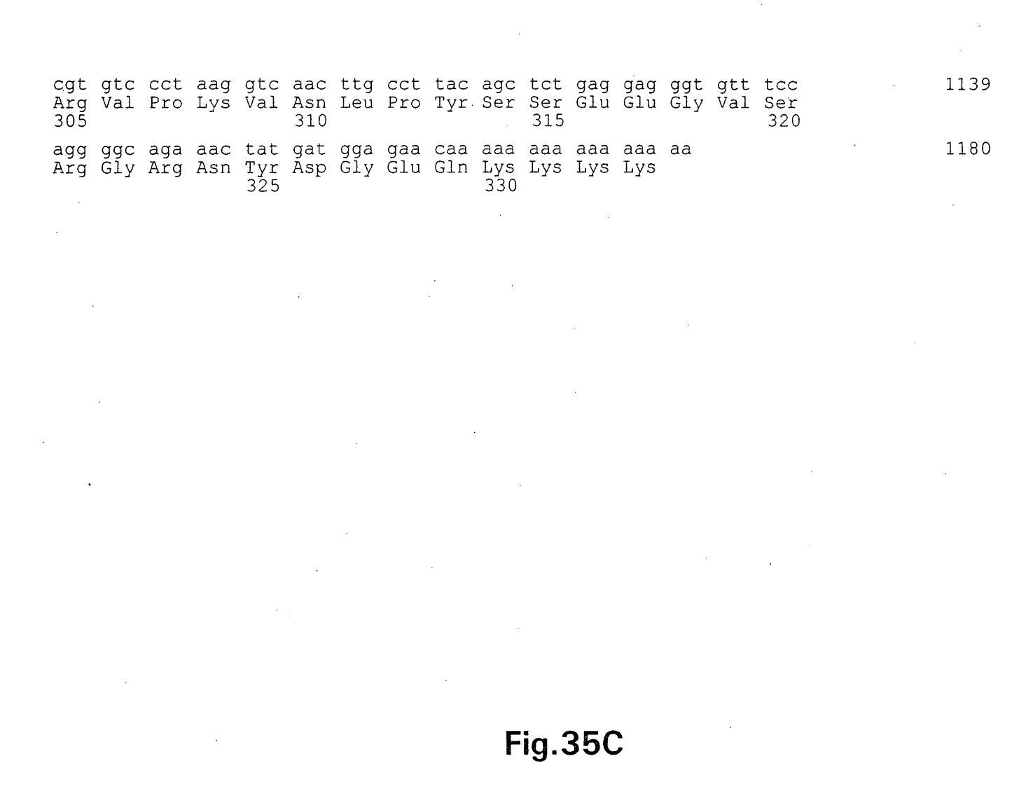

A mouse homolog of human TANGO 212 was identified. A cDNA encoding mouse TANGO 212 was identified by analyzing the sequences of clones present in a mouse osteob last LPS stimulated cDNA library. This analysis led to the identification of a clone, jtmoa103g01, encoding mouse TANGO 212. The mouse TANGO 212 cDNA of this clone is 1180 nucleotides long (FIG. 35A-35C; SEQ ID NO:25). The open reading frame of this cDNA (nucleotides 180 to 1179 of SEQ ID NO:25) encodes a polypeptide comprising a 334 amino acid secreted protein (SEQ ID NO:26).

In situ tissue screening was performed on mouse adult and embryonic tissue to analyze for the expression of mouse TANGO 212 mRNA. Of the adult tissues tested, only the renal medulla (kidney and medullary collecting tubules) was positive. Expression was observed primarily in the embryo. Signal was observed at E13.5 in the lung, skin (especially the upper lip), diaphragm, and muscle of the abdominal cavity and skin. This pattern remained through E18.5 with increasing lung expression. Muscle expression was still apparent at E18.5 but decreased to near background levels by postnatal day 1.5 with residual expression in the upper lip. No signal was detected in the following tissues: lung, diaphragm (smooth muscle), heart, liver, pancreas, thymus, eye, brain, bladder, small intestine, skeletal muscle, colon, placenta. In the case of embryonic mouse expression during the period of E13.5 through E16.5, expression was observed in the skin; especially upper lip/snout area, in the lung-multifocal at 13.5 but became more ubiquitous and more intense, muscle and diaphragm, skin, limbs (especially 13.5 and 14.5), and the abdominal wall. At E18.5, the expression observed was the same as for 13.5 through 16.5 but decreasing in muscle and skin (except upper lip). At P1.5, the expression signal decreased to almost background levels except in the upper lip.

Human and mouse TANGO 212 sequences exhibit considerable similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−4), reveals a protein identity of 77.2%. The human and mouse TANGO 212 cDNAs (SEQ ID NOs:13 and 25) are 80.5% identical, as assessed using the same software and parameters as indicated (without the BLOSUM 62 scoring matrix). In the respective open reading frames, calculated in the same fashion as the cDNAs, human and mouse TANGO 212 are 83.3% identical.

### Use of TANGO 212 Nucleic Acids, Polypeptides, and Modulators Thereof

The TANGO 212 proteins of the invention comprise a family of proteins having the hallmarks of a secreted protein of the EGF family. Accordingly, TANGO 212 proteins likely function in a similar manner as members of the EGF family. Thus, TANGO 212 modulators can be used to treat EGF-associated disorders.

For example, the TANGO 212 proteins likely play a role in tissue regeneration and/or wound healing. In vitro studies with several members of the EGF family such as EGF and TGF-α have shown that these proteins influence a number of cellular processes involved in soft tissue repair leading to their categorization as wound hormones in wound healing. The affects of these proteins include cellular proliferation and chemotaxis. Thus, the TANGO 212 proteins of the invention likely affect various cells associated with wound healing. Effects that the TANGO 212 proteins have on various cells include proliferation and chemotaxis. Accordingly, the TANGO 212 proteins, nucleic acids and/or modulators of the invention are useful in the treatment of wounds and/or the modulation of proliferative disorders, e.g., cancer.

Because TANGO 212 is expressed in the kidney, the TANGO 212 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such can be used to treat or modulate renal (kidney) disorders as discussed above in the section relating to uses of TANGO 140.

### TANGO 213

In another aspect, the present invention is based on the discovery of cDNA molecules which encode a novel family of proteins having sequence similarity to progesterone binding protein, referred to herein as TANGO 213 proteins.

Also included within the scope of the present invention are TANGO 213 proteins having a signal sequence.

In certain embodiments, a TANGO 213 family member has the amino acid sequence of SEQ ID NO: 16, and the signal sequence is located at amino acids 1 to 20, 1 to 22, 1 to 22, or 1 to 23. In such embodiments of the invention, the domains and the mature protein resulting from cleavage of such signal peptides are also included herein. For example, the cleavage of a signal sequence consisting of amino acids 1 to 22 results in a mature TANGO 213 protein corresponding to amino acids 23 to 371. The signal sequence is normally cleaved during processing of the mature protein.

In particular, BLASTP analysis using the amino acid sequence of TANGO 213 revealed sequence similarity between TANGO 213 and several steroid binding-proteins including 51% sequence identity between TANGO 213 and human progesterone binding protein (GenBank Accession No. Y12711). Thus, the TANGO 213 proteins of the invention are likely to function similarly to steroid binding-proteins. Steroid binding protein activities include the ability to form protein-protein interactions with steroid hormones in signaling pathways and/or the ability to modulate intracellular ion levels, e.g., sodium and/or calcium levels. Accordingly, TANGO 213 proteins, nucleic acids and/or modulators can be used to treat steroid binding protein-associated disorders.

### Human TANGO 213

A cDNA encoding human TANGO 213 was isolated by screening a human mesangial cell library. Human TANGO 213 comprises a 1496 nucleotide cDNA (16A-16C; SEQ ID NO: 15). It is noted that this nucleotide sequence contains Sal I and Not I adapter sequences on the 5′ and 3′ ends, respectively. The open reading frame of this cDNA (nucleotides 58 to 870 of SEQ ID NO: 15) encodes a 271 amino acid secreted protein (SEQ ID NO:16).

The signal peptide prediction program SIGNALP (Nielsen et al. (1997) Protein Engineering 10:1-6) predicted that human TANGO 213 includes a 22 amino acid signal peptide (amino acids 1 to about amino acid 22 of SEQ ID NO:16) preceding the mature TANGO 213 protein (corresponding to about amino acid 23 to amino acid 271 of SEQ ID NO: 16). Human TANGO 213 is predicted to have a molecular weight of approximately 29.5 kDa prior to cleavage of its signal peptide and a molecular weight of approximately 27.5 kDa subsequent to cleavage of its signal peptide.

A clone, EpDH156, which encodes human TANGO 213 was deposited with the American Type Culture Collection (ATCC®, 10801 University Boulevard, Manassas, Va. 20110-2209) on Oct. 30, 1998 and assigned Accession Number 98965. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

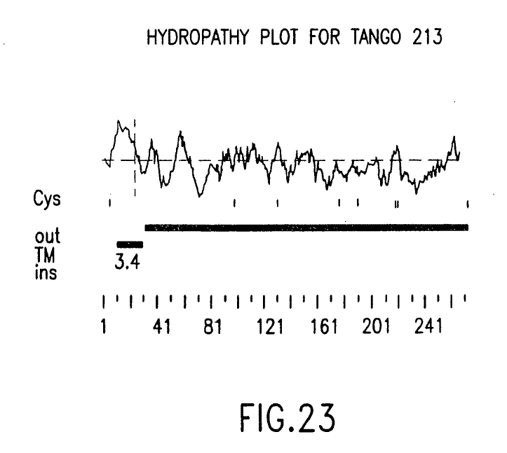

FIG. 23 depicts a hydropathy plot of human TANGO 213. As shown in the hydropathy plot, the hydrophobic region at the beginning of the plot which corresponds to about amino acids 1 to 22 is the signal sequence of TANGO 213.

Northern analysis of human TANGO 213 mRNA expression revealed expression at a very high level in testis and kidney. Expression at lower levels was also seen in all other tissues including adult heart, liver, brain, skeletal muscle, kidney, pancreas, spleen, thymus, prostate, ovary, small intestine, colon, and peripheral blood leukocytes. Low levels of expression were observed in lung.

The human gene for TANGO 213 was mapped on radiation hybrid panels to the long arm of chromosome 17, in the region p 13.3. Flanking markers for this region are WI-5436 and WI-6584. The MDCR (Miller-Dieker syndrome), PEDF (pigment epithelium derived factor), and PFN1 (profilin 1) genes also map to this region of the human chromosome. This region is syntenic to mouse chromosome 11, locus 46(g). The ti (tipsy) loci also maps to this region of the mouse chromosome. The pfn1 (profilin 1), htt (5-hydroxytryptamine (serotonin) transporter), acrb (acetylcholine receptor beta) genes also map to this region of the mouse chromosome.

### Mouse and Rat TANGO 213

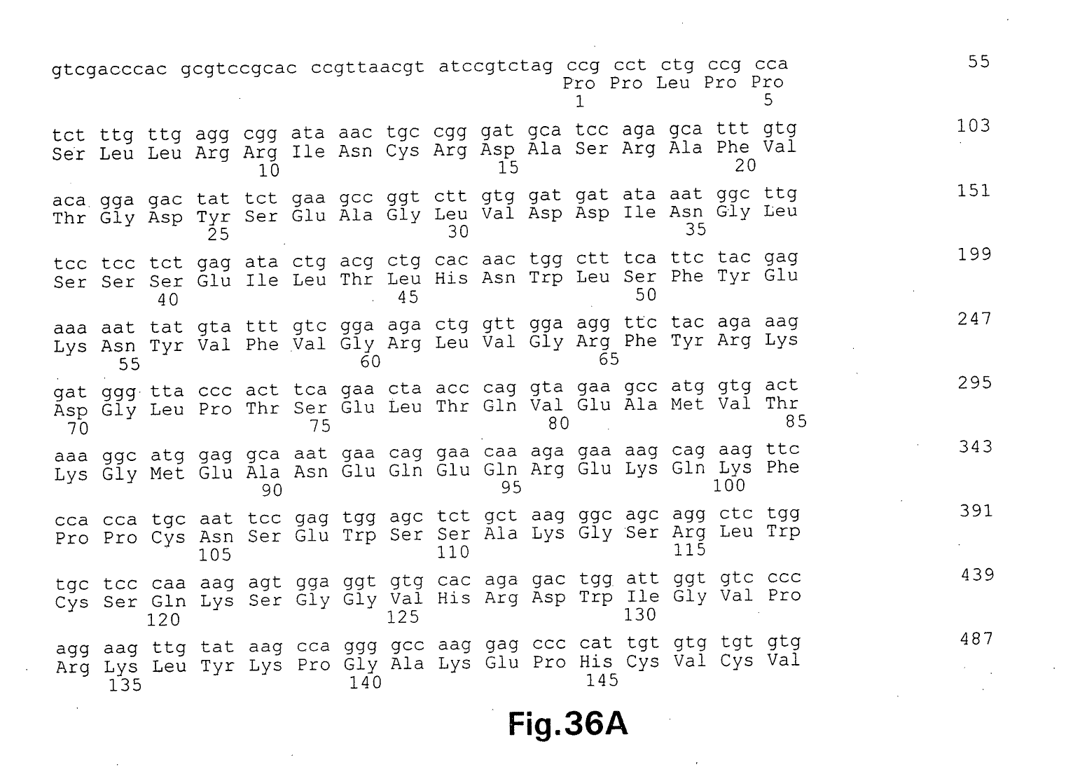

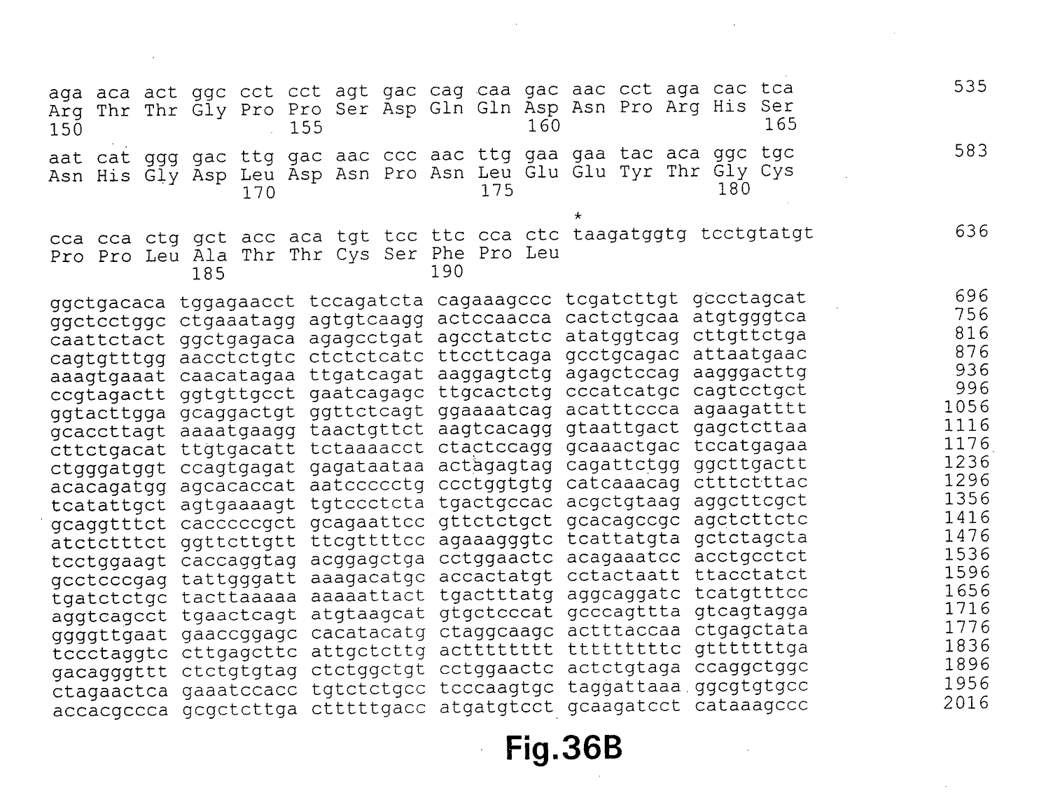

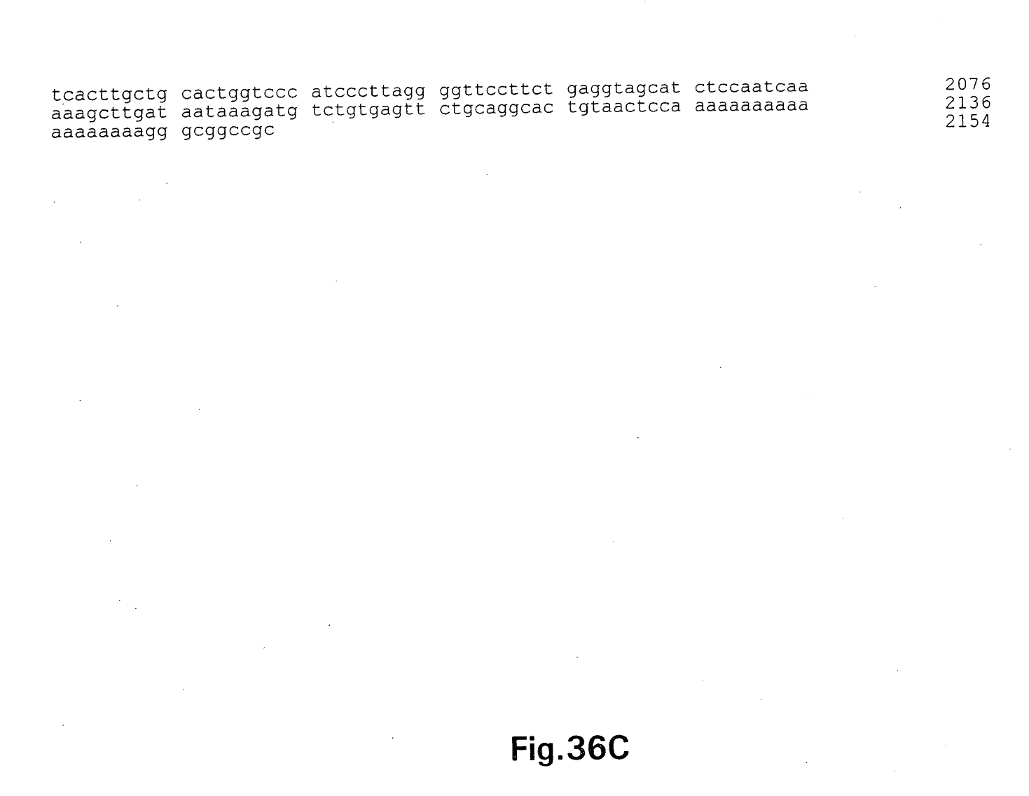

A mouse homolog of human TANGO 213 was identified. A cDNA encoding mouse TANGO 213 was identified by analyzing the sequences of clones present in a mouse testis cDNA library. This analysis led to the identification of a clone, jtmz213a01, encoding mouse TANGO 213. The mouse TANGO 213 cDNA of this clone is 2154 nucleotides long (FIG. 36A-36C; SEQ ID NO:27). It is noted that the nucleotide sequence contains a Not I adapter sequence on the 3′ end. The open reading frame of this cDNA (nucleotides 41 to 616 of SEQ ID NO:27) encodes a protein comprising the 192 amino acid sequence protein (SEQ ID NO:28).

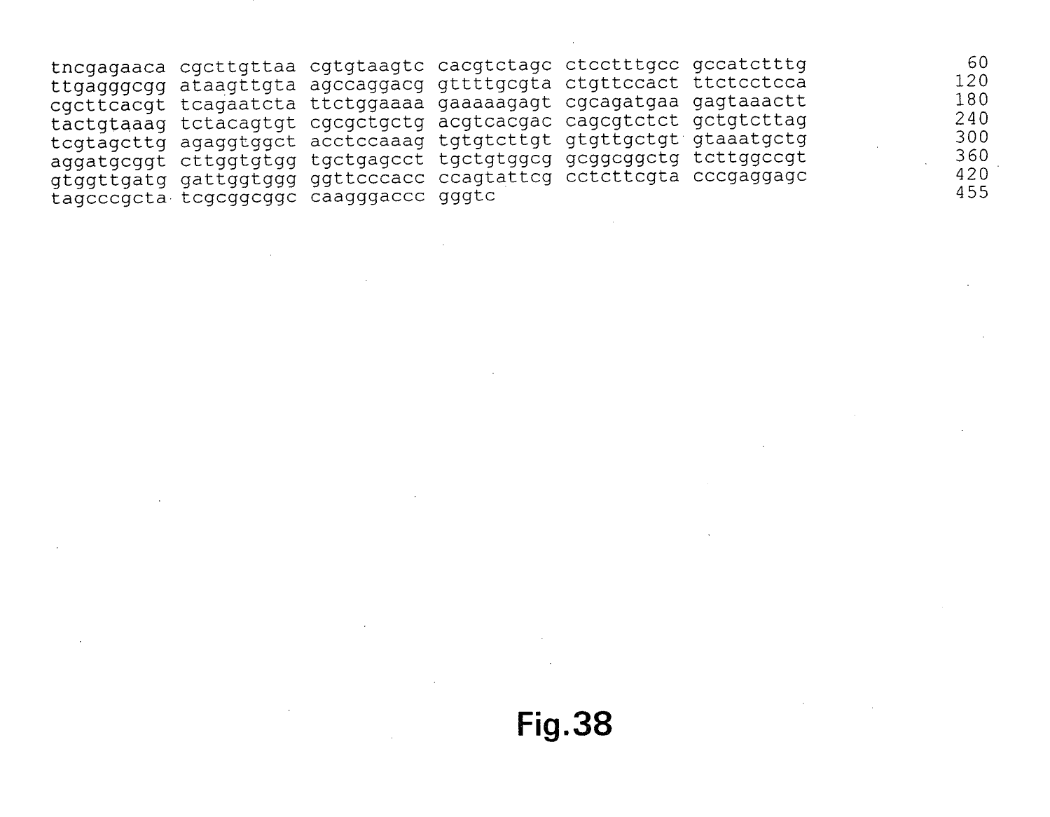

A rat homolog of human TANGO 213 was identified. A cDNA encoding rat TANGO 213 was identified by analyzing the sequences of clones present in a rat testis cDNA library. This analysis led to the identification of a clone encoding rat TANGO 213. The rat TANGO 213 cDNA of this clone is 455 nucleotides long (FIG. 38; SEQ ID NO:29). A translation of one open reading frame from the rat cDNA is shown in SEQ ID NO:30.

In situ tissue screening was performed on mouse adult and embryonic tissue to analyze for the expression of mouse TANGO 213 mRNA. The strongest expression was observed in the seminiferous tubules of the testes. Moderate or weak expression is observed in several other adult tissues including the liver, kidney, and placenta. A weak, ubiquitous signal was observed in brain, heart, liver, kidney, adrenal gland, and the spleen. A signal was observed in the ovaries. A ubiquitous signal was seen in the labyrinth zone and slightly higher signal in the zone of giant cells. No signal was detected in the following tissues: spinal cord, eye and harderian gland, submandibular gland, white fat, brown fat, stomach, lung, colon, small intestine, thymus, lymph node, pancreas, skeletal muscle, and bladder. Embryonic expression is negligible. A weak signal was observed in the developing liver and CNS. The signal in the CNS was near background levels. Specifically, at E13.5, a weak, ubiquitous signal observed in the liver. At E14.5 and E15.5, a weak, ubiquitous signal was observed in the liver, brain, and spinal cord. At E16.5, E18.5 and P1.5, the signal in liver and CNS was even less pronounced and was almost at background levels. Library array expression studies were carried out as described above for mouse TANGO 197. Strong expression was detected in the choroid plexus 12.5 day whole mouse embryo, TM4 (Sertoli cells), from testis, esophagus, and kidney fibrosis library. Weak expression was detected in LPS-stimulated osteoblast tissue, 10.5 day whole mouse embryo, and in 11.5 day whole mouse embryo. No expression was detected in differential 3T3, 10.5 day mouse fetus, mouse kidney fibrosis model nephrotoxic serum (NTS), LPS-stimulated heart, LPS-stimulated osteoblasts, lung, mouse insulinoma (Nit-1), mouse normal/hyperplastic islets (pancreas), normal spleen, 11.5 day mouse, LPS-stimulated lung, Lung, LPS-stimulated osteoblasts, BL6 Lung, day 15, 3 hour inflammation model, BDL Day 10 (balb C liver), hypertropic heart, LPS-stimulated lung, LPS-stimulated kidney, LPS-stimulated lymph node, Balb C liver (bile duct ligation d2), mc/9 mast cells, 13.5 day mouse, LPS-stimulated anchored heart, normal thymus, Th2-ovarian-Tg, Balb C liver (bile duct ligation d2), mc/9 mast cells, normal heart, brain polysome (MPB), LPS-stimulated anchored liver, brain (EAE d10 model), th1-ovarian-Tg, heart, hypothalamus, lone term bone, marrow cells, LPS-stimulated lung, megakaryocyte, LPS-stimulated spleen, hyphae-stimulated long term bone marrow, lung, angiogenic pancreatic islets, Th2, brain, LPS-stimulated thymus, LPS-stimulated microglial cells, testes, tumor pancreatic islets, LPS-stimulated brain, LPS-stimulated alveolar macrophage cell line, mouse lung bleomycin model d7, pregnant uterus, and hypothalamus nuclei.

Human and mouse TANGO 213 sequences exhibit considerable similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−4), reveals a protein identity of 64.6%. The human and mouse TANGO 213 cDNAs are 68.8% identical (SEQ ID NOs:15 and 27), as assessed using the same software and parameters as indicated (without the BLOSUM 62 scoring matrix). In the respective ORFs, calculated in the same fashion as the cDNAs, human and mouse TANGO 213 are 77.1%. identical.

### Uses of TANGO 213 Nucleic Acids, Polypeptides, and Modulators Thereof

The TANGO 213 proteins and nucleic acid molecules of the invention have at least one “TANGO 213 activity” (also referred to herein as “TANGO 213 biological activity”). TANGO 213 activity refers to an activity exerted by a TANGO 213 protein or nucleic acid molecule on a TANGO 213 responsive cell in vivo or in vitro. Such TANGO 213 activities include at least one or more of the following activities: 1) interaction of a TANGO 213 protein with a TANGO 213-target molecule; 2) activation of a TANGO 213 target molecule; 3) modulation of cellular proliferation; 4) modulation of cellular differentiation; or 5) modulation of a signaling pathway. Thus, the TANGO 213 proteins, nucleic acids and/or modulators can be used for the treatment of a disorder characterized by aberrant TANGO 213 expression and/or an aberrant TANGO 213 activity, such as proliferative and/or differentiative disorders.

As TANGO 213 is expressed in the kidney, the TANGO 213 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such can be used to treat or modulate renal (kidney) disorders as discussed above in the section relating to uses of TANGO 140.

Furthermore, as TANGO 213 is expressed in the testis, the TANGO 213 polypeptides, nucleic acids and/or modulators thereof can be used as discussed above in the section relating to uses of TANGO 128.

### TANGO 224

In another aspect, the present invention is based on the discovery of cDNA molecules which encode a novel family of proteins referred to herein as TANGO 224 proteins.

For example, the TANGO 224 proteins of the invention include a thrombospondin type I (TSP-I) domain. The TSP-I domain is involved in the binding to both soluble and matrix macromolecules (e.g., sulfated glycoconjugates). As used herein, a thrombospondin type I (TSP-I) domain refers to an amino acid sequence of about 30 to about 60, preferably about 35 to 55, 40 to 50, and more preferably about 45 amino acids in length. TANGO 224 has such a signature pattern at about amino acids 42 to 81.

Conserved amino acid motifs, referred to herein as “consensus patterns” or “signature patterns”, can be used to identify TANGO 224 family members having a TSP-I domain. For example, the following signature pattern can be used to identify TANGO 224 family members: W-S-x-C-[SD]-x (2)-C-x (2)-G-x (3, 5)-R-x (7,15)-C-x (9, 11)-C-x (4, 5)-C. A TSP-I domain of TANGO 224 extends, for example, from about amino acids 37 to 81 (SEQ ID NO:18).

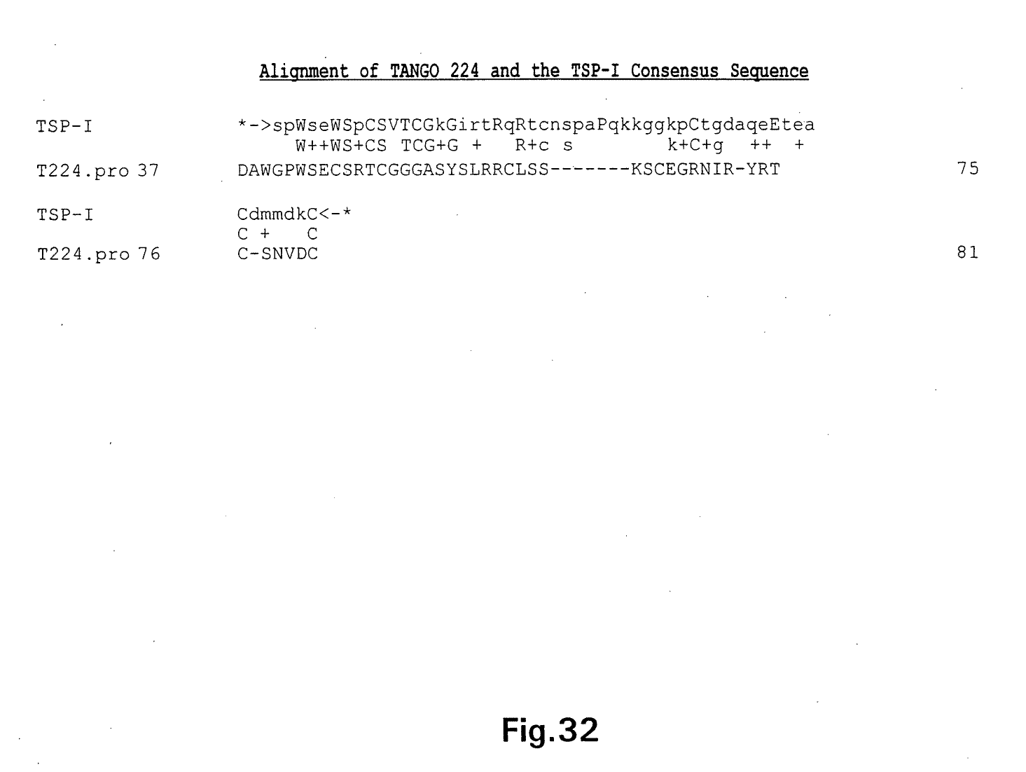

A TSP-I domain further contains at least about 4 to 9, preferably, 5 to 8, more preferably 6 conserved cysteine residues. By alignment of a TANGO 224 family member with a TSP-I consensus sequence, conserved cysteine residues can be found. For example, as shown in FIG. 32, there is a first cysteine residue in the TSP-I consensus sequence that corresponds to a cysteine residue at amino acid 45 of TANGO 224; there is a second cysteine residue in the TSP-I consensus sequence that corresponds to a cysteine residue at amino acid 49 of TANGO 224; there is a third cysteine residue in the TSP-I consensus sequence that corresponds to a cysteine residue at amino acid 60 of TANGO 224; there is a fourth cysteine residue in the TSP-I consensus sequence that corresponds to a cysteine residue at amino acid 66 of TANGO 224; there is a fifth cysteine residue in the TSP-I consensus sequence that corresponds to a cysteine residue at amino acid 76 of TANGO 224; and/or there is a sixth cysteine residue in the TSP-I consensus sequence that corresponds to a cysteine residue at amino acid 81 of TANGO 224. The TSP-I consensus sequence is available from the KHMer version 2.0 software as Accession Number PF00090. Software for HMM-based profiles is available from http://www.csc.ucsc.edu/research/compbio/sam.html and from http://genome.wustl.edu/eddy/hmmer.html.

For example, the TANGO 224 proteins of the invention include a Furin-like cysteine rich domain (Accession number:PF00757). The consensus sequence for the Furin-like cysteine rich domain is: C-Xaa(3)-C-Xaa-G-G-Xaa(n)-C-Xaa(5)-D-G, wherein C is cysteine, Xaa is any amino acid, G is glycine, n is about 5 to 15, preferably 6 to 14, more preferably about 7 to 12, and D is aspartic acid. As used herein, a Furin-like cysteine rich domain refers to an amino acid sequence of about 80 to 160, preferably of about 100 to 150, and more preferably about 110 to 130, amino acids in length. Human TANGO 224, form 2 has such a signature pattern at about amino acids 707-829 (SEQ ID NO:20). Also included within the scope of the present invention are TANGO 224 proteins having a signal sequence.

In certain embodiments, a TANGO 224 family member has the amino acid sequence of SEQ ID NO:18, and the signal sequence is located at amino acids 1 to 26, 1 to 27, 1 to 28, 1 to 29 or 1 to 30. In such embodiments of the invention, the domains and the mature protein resulting from cleavage of such signal peptides are also included herein. For example, the cleavage of a signal sequence consisting of amino acids 1 to 28 results in a mature TANGO 224, form 1 protein corresponding to amino acids 29 to 458 of SEQ ID NO: 18. The signal sequence is normally cleaved during processing of the mature protein.

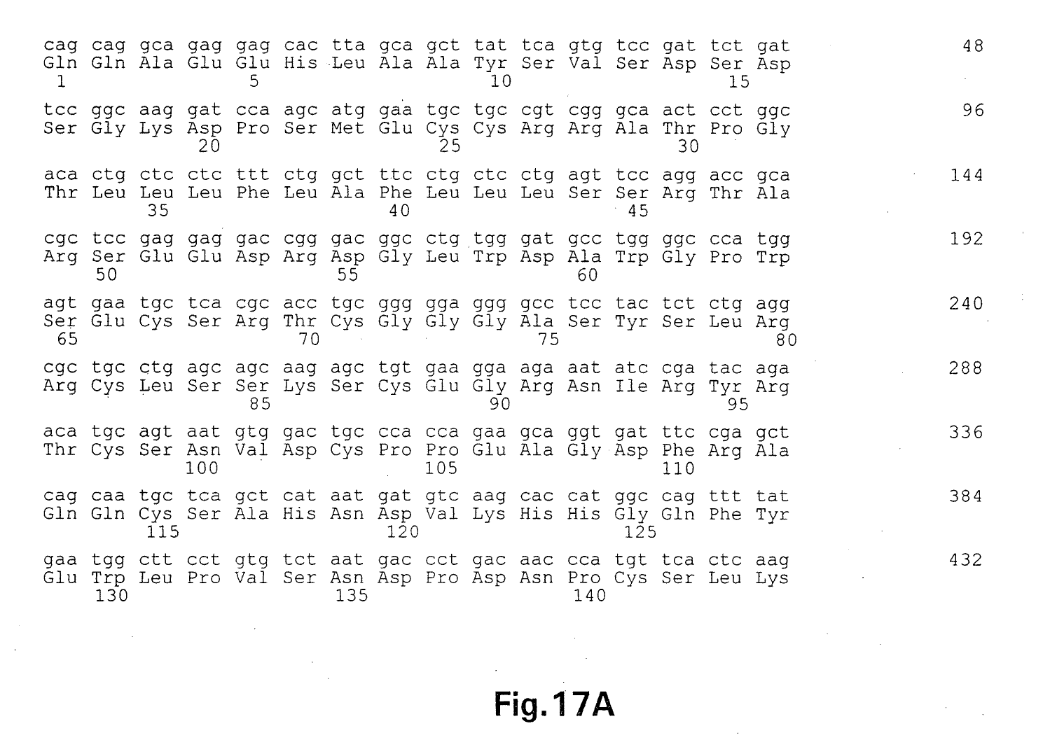

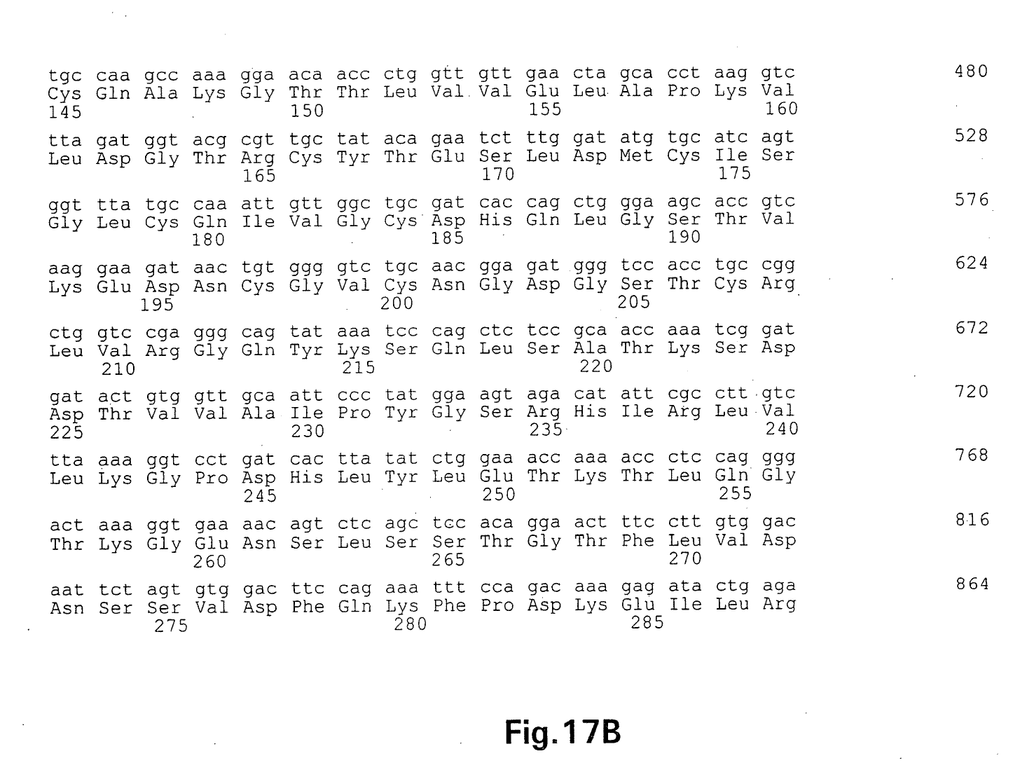

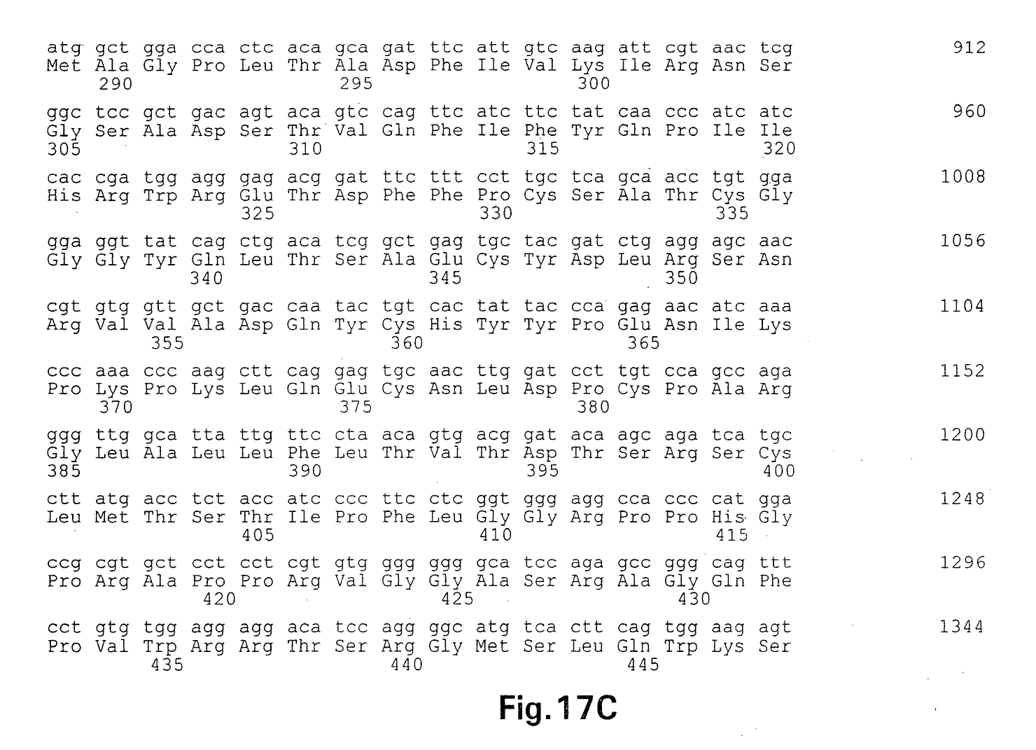

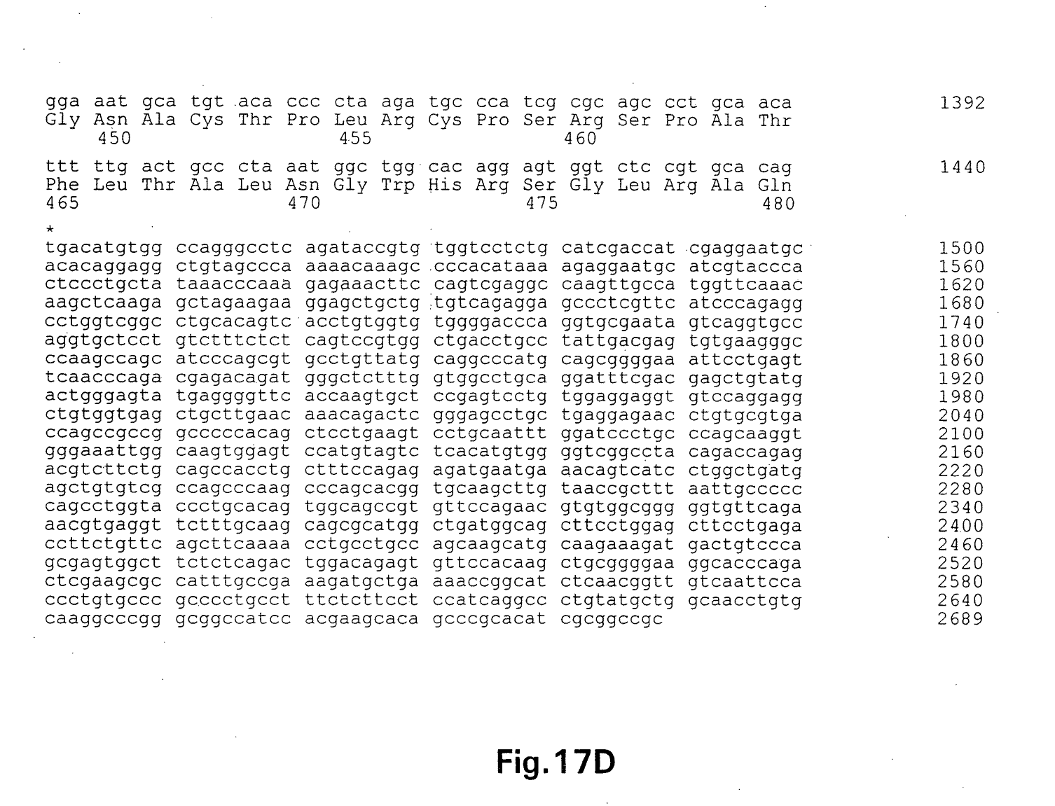

A cDNA encoding human TANGO 224 was identified by screening a human fetal spleen library. A clone comprising human TANGO 224 was selected for complete sequencing. In one embodiment, TANGO 224 is referred to as TANGO 224, form 1. Human TANGO 224, form 1 comprises a 2689 nucleotide cDNA (FIG. 17A-17D; SEQ ID NO: 17). The open reading frame of this TANGO 224, form 1 cDNA clone (nucleotides 1 to 1440 of SEQ ID NO: 17) and encodes a secreted-protein comprising the 480 amino acid sequence (SEQ ID NO: 18).

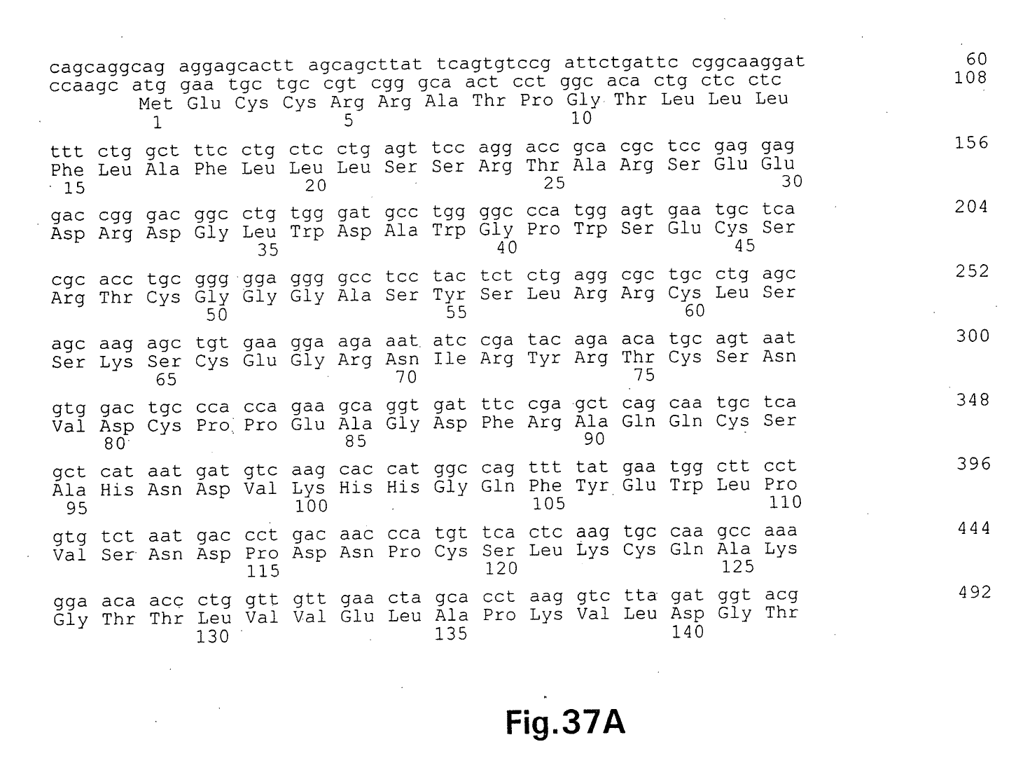

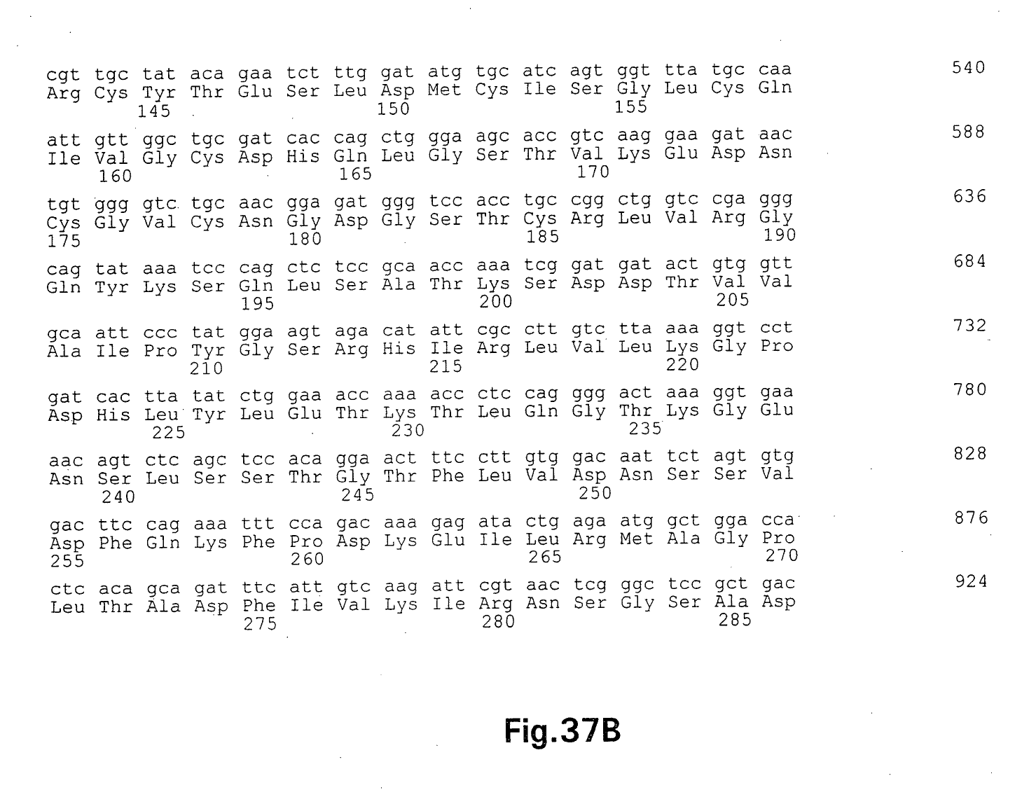

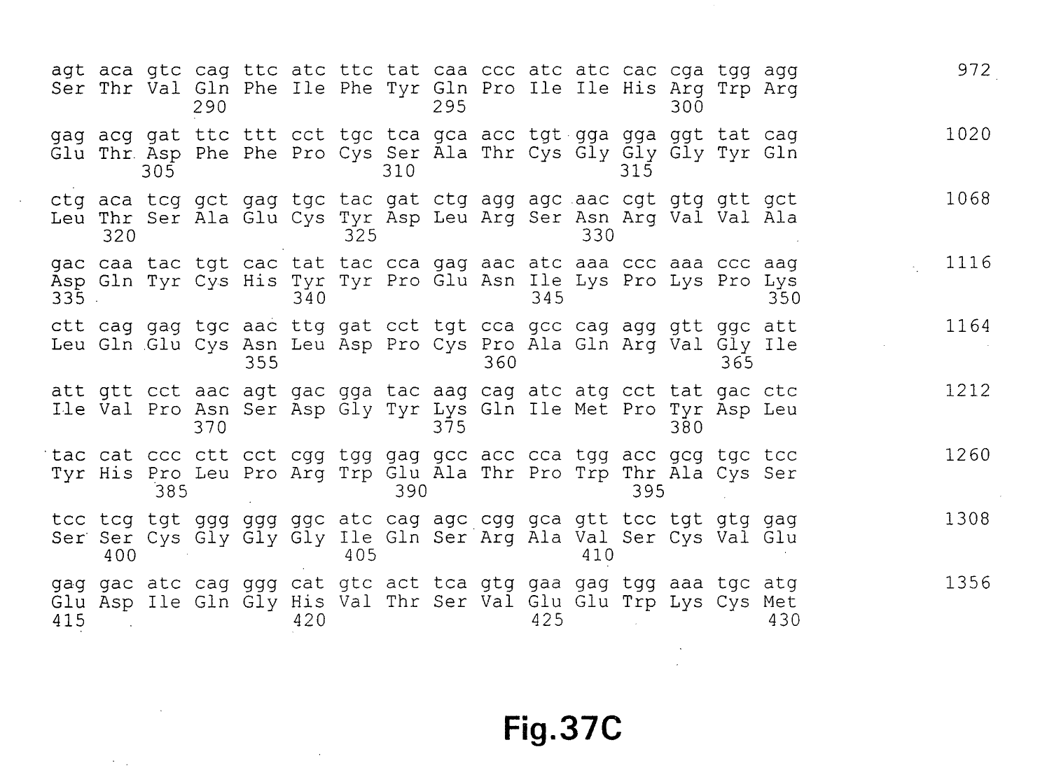

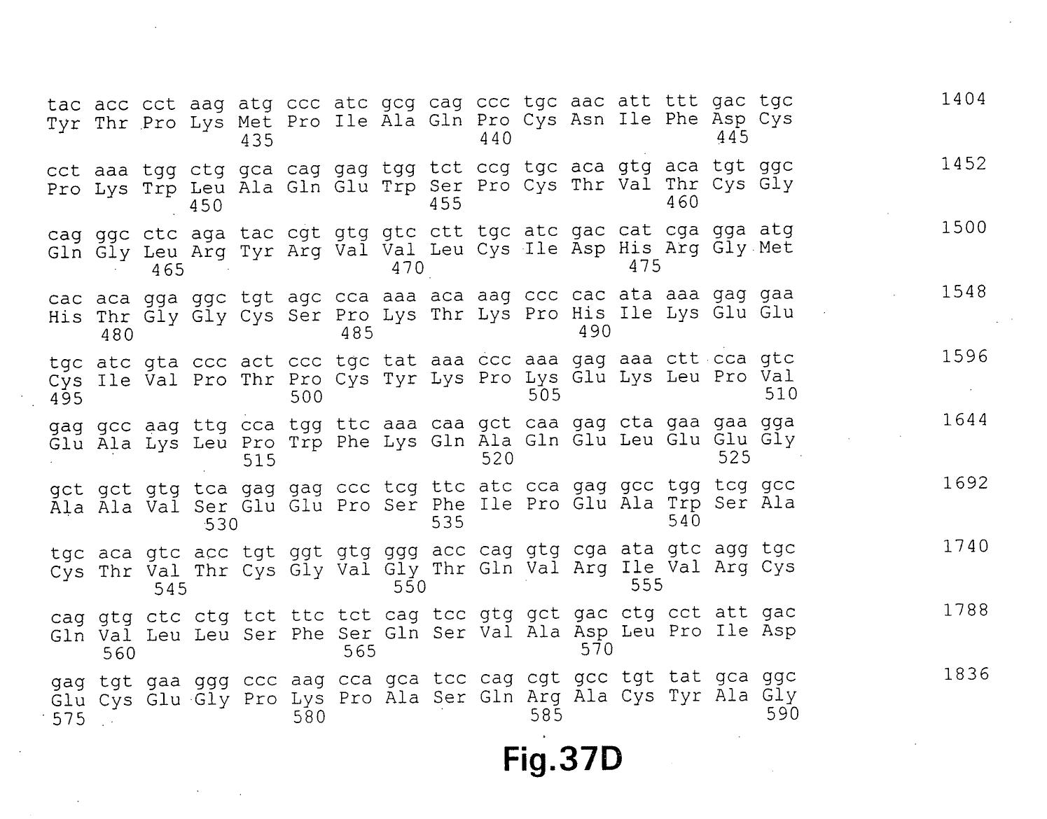

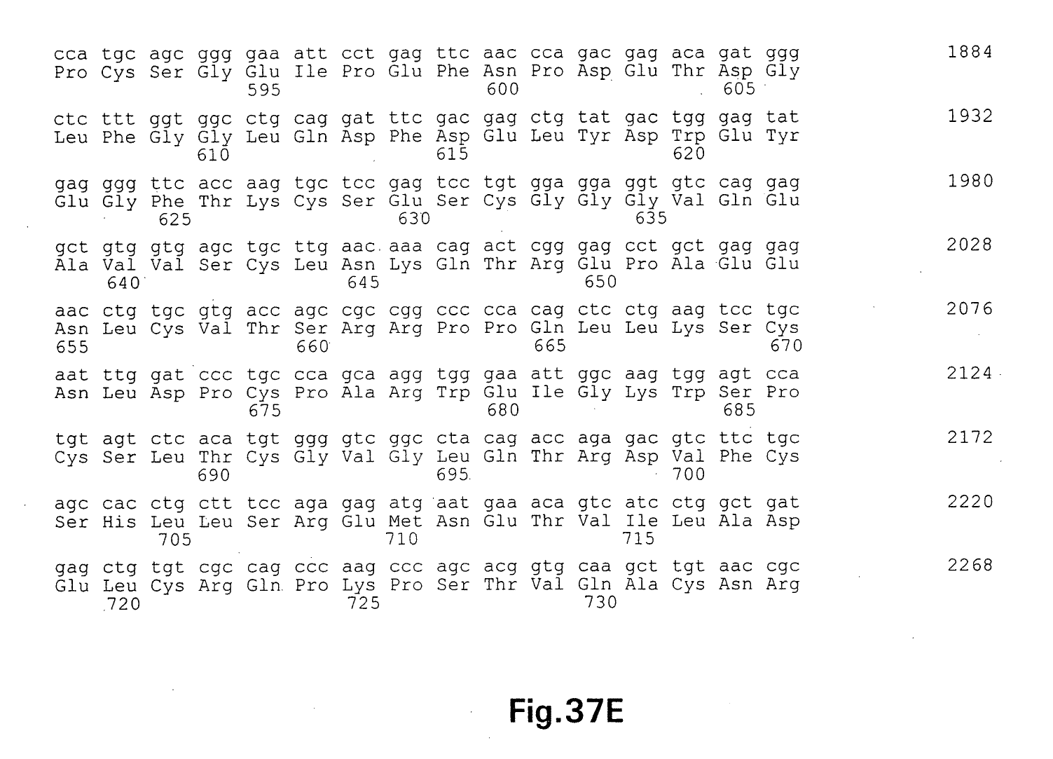

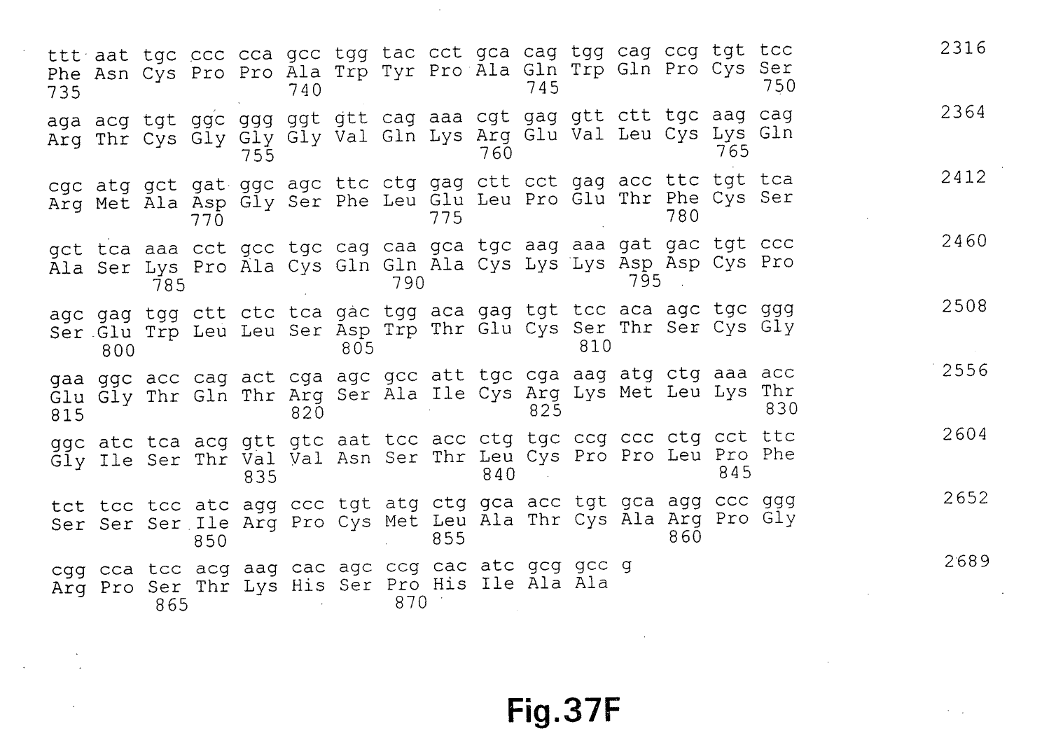

Another cDNA clone comprising human TANGO 224, was also obtained. This TANGO 224 clone comprises a 2691 nucleotide cDNA (FIG. 37A-37F; SEQ ID NO: 19), and encodes a human TANGO 224 and is referred to as human TANGO 224, form 2. The open reading frame of human TANGO 224, form 2 cDNA clone (nucleotides 67 to 2690 of SEQ ID NO: 19) and encodes a secreted protein comprising the 874 amino acid protein (SEQ ID NO:20).

The signal peptide prediction program SIGNALP (Nielsen et al. (1997) Protein Engineering 10:1-6) predicted that human TANGO 224 form 1 includes an 28 amino acid signal peptide (amino acids 1 to about amino acid 28 of SEQ ID NO: 18) preceding the mature TANGO 224 protein (corresponding to about amino acid 29 to amino acid 458 of SEQ ID NO: 18). Human TANGO 224 is predicted to have a molecular weight of approximately 50 kDa prior to cleavage of its signal peptide and a molecular weight of approximately 47 kDa subsequent to cleavage of its signal peptide.

The signal peptide prediction program SIGNALP (Nielsen et al. (1997) Protein Engineering 10:1-6) predicted that human TANGO 224 form 2 includes an 28 amino acid signal peptide (amino acids 1 to about amino acid 28 of SEQ ID NO:20) preceding the mature TANGO 224, form 2 protein (corresponding to about amino acid 29 to amino acid 874 of SEQ ID NO:20). Human TANGO 224 is predicted to have a molecular weight of approximately 131 kDa prior to cleavage of its signal peptide and a molecular weight of approximately 127 kDa subsequent to cleavage of its signal peptide.

Human TANGO 224, form 1 has a TSP-I domain from about amino acids 37 to 81 of SEQ ID NO:18. Human TANGO 224, form 2 has a TSP-I domain from about amino acids 37 to 81 of SEQ ID NO:20.

Human TANGO 224, form 2 has a Furin-like cysteine rich domain from amino acids 707 to 829 of SEQ ID NO:20.

A clone, EpDH210, which encodes human TANGO 224, form 1 was deposited with the American Type Culture Collection (ATCC®, 10801 University Boulevard, Manassas, Va. 20110-2209) on Oct. 30, 1998 and was assigned Accession Number 98966. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

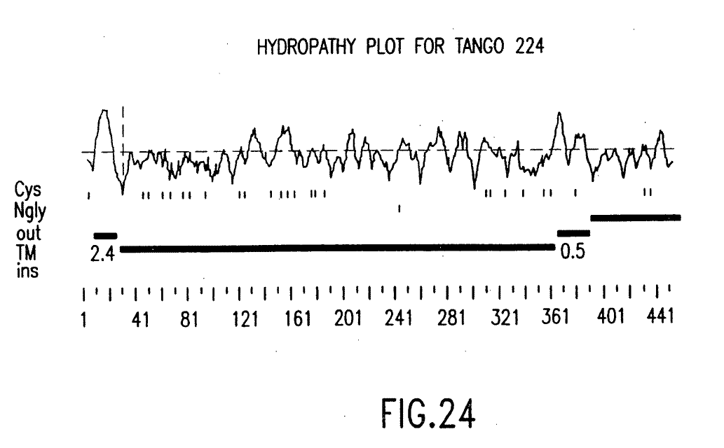

FIG. 24 depicts a hydropathy plot of human TANGO 224. As shown in the hydropathy plot, the hydrophobic region at the beginning of the plot which corresponds to about amino acids 1 to 28 is the signal sequence of TANGO 224.

Northern analysis of human TANGO 224 mRNA expression using TANGO 224 form 2 nucleotide sequence as a probe revealed expression of TANGO 224 mRNA in the spleen, prostate, ovary and colon. Only weak expression was detected in testis, small intestine, and peripheral blood leukocytes. No expression was detected in the thymus.

Library Array Expression studies were performed as described above for the mouse TANGO 128 gene, except that human tissues were tested. Strong expression was obtained in the pituitary and fetal spleen. Only weak expression was detected in the primary osteoblasts, umbilical smooth muscle treated and the bronchial smooth muscle. No expression was detected in kidney, testes, Prostate, HMC-1 control (mast cell line), fetal dorsal spinal cord, human colon to liver metastasis, erythroblasts from CD34+Blood, human spinal cord (ION 3), HUVEC TGF-B (h. umbilical endothelia), HUVEC (h. umbilical endothelia), human spinal cord (ION 3), brain K563 (red blood cell line), uterus, Hep-G2 (human insulinoma), human normal colon, human colon to liver metastasis, skin, HUVEC controls (umbilical endothelial cells), human colon (inflammatory bowel disease), melanoma (G361 cell line), adult bone arrow CD34+ cells, HPK, human lung, mammary gland, normal breast epithelium, colon to liver metastasis (CHT128), normal breast, bone marrow (CD34+), W138 (H. embryonic Lung), Th1 cells, HUVEC untreated (umbilical endothelium), liver, spleen, normal human ovarian epithelia, colon to liver metastasis (CHT133), PTH-treated osteoblasts, ovarian ascites, lung squamous cell, carcinoma (MDA 261), Th2 cells, colon (WUM 23), thymus, heart, small intestine, normal megakaryoctyes, colon carcinoma (NDR109), lung adenocarcinoma (PIT245), IBD Colon (WUM6), brain-subcortical white matter (ION2), prostate tumor xenograft A12, trigeminal ganglia 9 week fetus, thymus, retinal pigmentosa epithelia, bone marrow, colon carcinoma (NDR103), lung squamous cell carcinoma (PIT299), cervical cancer, normal prostate, Prostate tumor xenograft K, Lumbrosacaral spinal cord, A549 control, stomach, retina, Th-1 induced T cell, colon carcinoma (NDR82), d8 dendritic ells, spinal cord, ovarian epithelial tumor, prostate cancer to liver metastasis JHH3, lumbrosacaral dorsal root ganglia, salivary gland, skeletal muscle, HMC-1 (human mast cell line), Th-2 induced T-cell, colon carcinoma (NDR097), H6. megakaryocytes, H7. dorsal root ganglia (ION 6, 7, 8), H8. HUVEC L-NAME (umbilical endothelia), H9. prostate cancer to liver metastasis JHH4, H10. Dorsal root ganglia (ION 6, 7, 8),

### Use of TANGO 224 Nucleic Acids, Polypeptides, and Modulators Thereof

As discussed above, the TSP-I domain of TANGO 224 is involved in matrix interactions. Thus, the TANGO 224 proteins of the invention likely play a role in various matrix interactions, e.g., matrix binding. Thus, a TANGO 224 activity is at least one or more of the following activities: 1) regulation of extracellular matrix structuring; 2) modulation of cellular adhesion, either in vitro or in vivo; 3) regulation of cell trafficking and/or migration. Accordingly, the TANGO 224 proteins, nucleic acid molecules and/or modulators can be used to modulate cellular interactions such as cell-cell and/or cell-matrix interactions and thus, to treat disorders associated with abnormal cellular interactions.

As TANGO 224 was originally found in a fetal spleen library, TANGO 228 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 224 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 224 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

### HtrA-2 (TANGO 214)

The HtrA-2 proteins and nucleic acid molecules comprise a family of molecules having certain conserved structural and functional features. For example, HtrA-2 proteins of the invention have signal sequences. Thus, in one embodiment, an HtrA-2 protein contains a signal sequence of about amino acids 1 to 17. The signal sequence is normally cleaved during processing of the mature protein.

HtrA-2 family members can also include an IGF-binding domain. As used herein, the term “IGF-binding domain” refers to a cysteine rich protein domain that includes about 40-80 amino acid residues, preferably about 50-70 amino acid residues, more preferably about 55-65 amino acid residues, and most preferably about 61 amino acid residues. Typically, an IGF-binding domain is found at the N-terminal half of HtrA-2 and includes a cluster of about 6-15 cysteine residues conserved in IGF binding protein family members, more preferably about 8-10 cysteine residues, and still more preferably about 11 cysteine residues. In addition, an IGF-binding domain includes at least the following consensus sequence: C-Xaa-C-C-Xaa(n1)-C-Xaa-Xaa(n2)-C, wherein C is a cysteine residue, Xaa is any amino acid, n1 is about 1-5 amino acid residues, more preferably about 1-3 amino acid residues, and more preferably 2 amino acid residues in length, and n2 is about 2-10 amino acid residues, more preferably 5-10 amino acid residues, and more preferably 6 amino acid residues in length. In a preferred embodiment, an IGF-binding domain includes at least the following consensus sequence: C-Xaa-C-C-Xaa(n1)-C-A-Xaa(n2)-C, wherein C is a cysteine residue, Xaa is any amino acid, n1 is about 1-5 amino acid residues, more preferably about 1-3 amino acid residues, and more preferably 2 amino acid residues in length, and n2 is about 2-10 amino acid residues, more preferably 5-10 amino acid residues, and more preferably 6 amino acid residues in length.

In one embodiment, an HtrA-2 family member includes an IGF-binding domain having an amino acid sequence that is at least about 55%, preferably at least about 65%, more preferably at least about 75%, yet more preferably at least about 85%, and most preferably at least about 95% identical to amino acids 18 to 78, which is the IGF-binding domain of HtrA-2. In another embodiment, an HtrA-2 family member includes an IGF-binding domain having an amino acid sequence that is at least about 55%, preferably at least about 65%, more preferably at least about 75%, yet more preferably at least about 85%, and most preferably at least about 95% identical to amino acids 18 to 78, includes a conserved cluster of 11 cysteine residues, and an IGF-binding domain consensus sequence as described herein. In yet another embodiment, an HtrA-2 family member includes an IGF-binding domain having an amino acid sequence that is at least 55%, preferably at least about 65%, more preferably at least about 75%, yet more preferably at least about 85%, and most preferably at least about 95% identical to amino acids 18 to 78, includes a conserved cluster of 11 cysteine residues, an IGF-binding domain consensus sequence as described herein, and has at least one HtrA-2 biological activity as described herein.

In a preferred embodiment, an HtrA-2 family member has the amino acid sequence of SEQ ID NO:32 wherein the cluster of conserved cysteine residues is located within amino acid residues 25 to 76 (at positions, 25, 29, 34, 39, 48, 50, 51, 54, 62, 70, and 76 of SEQ ID NO:32), and the IGF-binding domain consensus-sequence is located at amino acid residues 48 to 62 of SEQ ID NO:32.

An HtrA-2 family member can also include a Kazal protease inhibitor domain. As used herein, the term “Kazal protease inhibitor domain” refers to a protein domain that includes about 30-70 amino acid residues, preferably about 40-60 amino acid residues, more preferably about 45-55 amino acid residues, and most preferably about 48 amino acid residues. Typically, a Kazal protease inhibitor domain includes a conserved tyrosine residue and a conserved cluster of about 3-7 cysteine residues, preferably about 4-6 cysteine residues, and still more preferably about 5 cysteine residues. In addition, a Kazal serine protease inhibitor domain includes at least the following consensus sequence: C-Xaa(n1)-C-Xaa(n2)-Y-Xaa(3)-C, wherein C is a cysteine residue, Xaa is any amino acid, n1 is about 4-amino acid residues in length, more preferably about 5-8 amino acid residues, and most preferably about 6 amino acid residues in length, n2 is about 4-10 amino acid residues, more preferably about 5-8 amino acid residues, and most preferably about 6 amino acid residues in length, Y is a tyrosine residue, and 3 represents a length of 3 amino acid residues of the type preceding it (in this case 3 of any amino acid (Xaa)).

In one embodiment, an HtrA-2 family member includes a Kazal protease inhibitor domain having an amino acid sequence that is at least about 55%, preferably at least about 65%, more preferably at least about 75%, yet more preferably at least about 85%, and most preferably at least about 95% identical to amino acids 79 to 126. In another embodiment, an HtrA-2 family member includes a Kazal protease inhibitor domain having an amino acid sequence that is at least about 55%, preferably at least about 65%, more preferably at least about 75%, yet more preferably at least about 85%, and most preferably at least about 95% identical to amino acids 79 to 126, includes a cluster of 5 cysteine residues, and a Kazal protease inhibitor domain consensus sequence as described herein. In yet another embodiment, an HtrA-2 family member includes a Kazal protease inhibitor domain having an amino acid sequence that is at least about 55%, preferably at least about 65%, more preferably at least about 75%, yet more preferably at least about 85%, and most preferably at least about 95% identical to amino acids 79 to 126, includes a cluster of 5 cysteine residues, and a Kazal protease inhibitor domain consensus sequence as described herein, and has at least one HtrA-2 biological activity as described herein.

In a preferred embodiment, an HtrA-2 family member has the amino acid sequence of SEQ ID NO:32 wherein the cluster of 5 cysteine residues is located within amino acid residues 81 to 126 (at positions 81, 83, 90, 101, and 126) and the Kazal protease inhibitor domain consensus sequence is located from amino acid residues 83 to amino acid residue 101 of SEQ ID NO:32.

An HtrA-2 family member can also include a serine protease domain. As used herein, the term “serine protease domain” refers to a protein domain that includes about 180-240 amino acid residues, preferably about 190-230 amino acid residues, more preferably about 205-215 amino acid residues, and most preferably about 208 amino acid residues. In addition, a serine protease domain includes a conserved serine residue, a conserved histidine residue, and a conserved aspartic acid residue in its active site. The conserved histidine, aspartic acid, and serine residues typically appear in the active site within three motifs: 1) a conserved histidine active site motif as follows: Thr-Asn-Xaa-His-Val, where Xaa represents Ala or Asn; 2) a conserved aspartic acid active site motif as follows: Asp-Ile-Ala-Xaa-Ile, where Xaa represents Leu or Thr; and 3) a conserved serine active site motif as follows: Gly-Asn-Ser-Gly-Gly-Xaa-Leu, where Xaa represents Pro or Ala. The conserved histidine active site motif is typically N-terminal to the conserved aspartic acid active site motif, which is N-terminal to the conserved serine active site motif. The histidine and aspartic acid motifs are typically separated from (noninclusive of the last amino acid residue of the first motif and the first residue of the subsequent motif) one another by at least about 15 to 55 amino acid residues, more preferably about 25 to 45 amino acid residues, still more preferably about 30 to 40 amino acid residues, and most preferably about 34 amino acid residues. The aspartic acid and serine motifs are typically separated from (noninclusive of the last amino acid residue of the first motif and the first residue of the subsequent motif) one another by at least about 50 to 90 amino acid residues, more preferably 60 to 80 amino acid residues, still more preferably about 65 to 78 amino acid residues, and most preferably about 71 amino acid residues.

In one embodiment, an HtrA-2 family member includes a serine protease domain having an amino acid sequence that is at least about 55%, preferably at least about 65%, more preferably at least about 75%, yet more preferably at least about 85%, and most preferably at least about 95% identical to amino acids 140 to 347, which is the serine protease domain of HtrA-2. In another embodiment, an HtrA-2 family member includes a serine protease domain having an amino acid sequence that is at least about 55%, preferably at least about 65%, more preferably at least about 75%, yet more preferably at least about 85%, and most preferably at least about 95% identical to amino acids 140 to 347 and includes a conserved histidine active site motif, a conserved aspartic acid active site motif, and a conserved serine active site motif as described herein. In yet another embodiment, an HtrA-2 family member includes a serine protease domain having an amino acid sequence that is at least about 55%, preferably at least about 65%, more preferably at least about 75%, yet more preferably at least about 85%, and most preferably at least about 95% identical to amino acids 140 to 347, includes a conserved histidine active site motif, a conserved aspartic acid active site motif, and a conserved serine active site motif as described herein, and has at least one HtrA-2 biological activity as described herein.

In a preferred embodiment, an HtrA-2 family member has the amino acid sequence of SEQ ID NO:32 wherein the conserved histidine active site motif is located at amino acid residues 188 to 192 (the histidine residue is at position 191), the conserved aspartic acid active site motif is located at amino acid residues 227 to 231 (the aspartic acid residue is at position 227), and the conserved serine active site motif is located at amino acid residues 303 to 309 (the serine residue is at position 305) of SEQ ID NO:32.

In one embodiment, an HtrA-2 family member can also include a PDZ domain. As used herein, the term “PDZ domain” refers to a protein domain that includes about 70-110 amino acid residues, preferably about 80-100 amino acid residues, more preferably about 87-97 amino acid residues, and most preferably about 92 amino acid residues. Typically, a PDZ domain is located at the C-terminal half of the HtrA-2 protein and includes at least about 3-7 conserved glycine residues, more preferably about 4-6 conserved glycine residues, and most preferably about 5 conserved glycine residues. Typically, a PDZ domain also includes at least the following consensus sequence of G-G-Xaa(n)-D-Xaa(n)-N-G, wherein G is glycine, Xaa is any amino acid, n is about 4-10 amino acid residues in length, more preferably about 5-8 amino acid residues in length, and more preferably about 5-6 amino acid residues in length, and D is aspartic acid.

In one embodiment, an HtrA-2 family member includes a PDZ domain having an amino acid sequence that is at least about 55%, preferably at least about 65%, more preferably at least about 75%, yet more preferably at least about 85%, and most preferably at least about 95% identical to amino acids 348 to 439. In another embodiment, an HtrA-2 family member includes PDZ domain having an amino acid sequence that is at least about 55%, preferably at least about 65%, more preferably at least about 75%, yet more preferably at least about 85%, and most preferably at least about 95% identical to amino acids 348 to 439 and is located at the C-terminal half of the protein and has a PDZ domain consensus sequence as described herein. In another embodiment, an HtrA-2 family member includes a PDZ domain having an amino acid sequence that is at least about 55%, preferably at least about 65%, more preferably at least about 75%, yet more preferably at least about 85%, and most preferably at least about 95% identical to amino acids 348 to 439, is located at the C-terminal half of the protein, includes about 5 conserved glycine residues, and has a PDZ domain consensus sequence as described herein. In yet another embodiment, an HtrA-2 family member includes a PDZ domain having an amino acid sequence that is at least about 55%, preferably at least about 65%, more preferably at least about 75%, yet more preferably at least about 85%, and most preferably at least about 95% identical to amino acids 348 to 439 of SEQ ID NO:32, is located at the C-terminal half of the protein, includes about 5 conserved glycine residues, has a PDZ domain consensus sequence as described herein, and has at least one HtrA-2 biological activity as described herein.

In a preferred embodiment, an HtrA-2 family member has the amino acid sequence of SEQ ID NO:32 wherein the PDZ domain is located at the C-terminal half of the protein, from amino acid residues 348 to 439, the PDZ domain consensus sequence is located from amino acid residues 400 to amino acid residue 413, and the conserved glycine residues are located within amino acid residues 358-413 (at positions 358, 385, 400, 401, and 413 of SEQ ID NO:32).

In another embodiment, the signal sequence and the IGF-binding domain of the HtrA-2 family member are adjacent (i.e., there are no intervening residues between the last residue of the signal sequence and the first residue of the IGF-binding domain) to one another. In another example, the IGF-binding domain and Kazal protease inhibitor domain are adjacent (i.e., there are no intervening residues between the last residue of the IGF-binding domain and the first residue of the Kazal protease inhibitor domain) to one another, and the IGF-binding domain is N-terminal to the Kazal protease inhibitor domain. In still another example, the signal sequence is adjacent and N-terminal to the IGF-binding domain, and the Kazal protease inhibitor domain is adjacent to and C-terminal to the IGF-binding domain.

### Human HtrA-2 (TANGO 214)

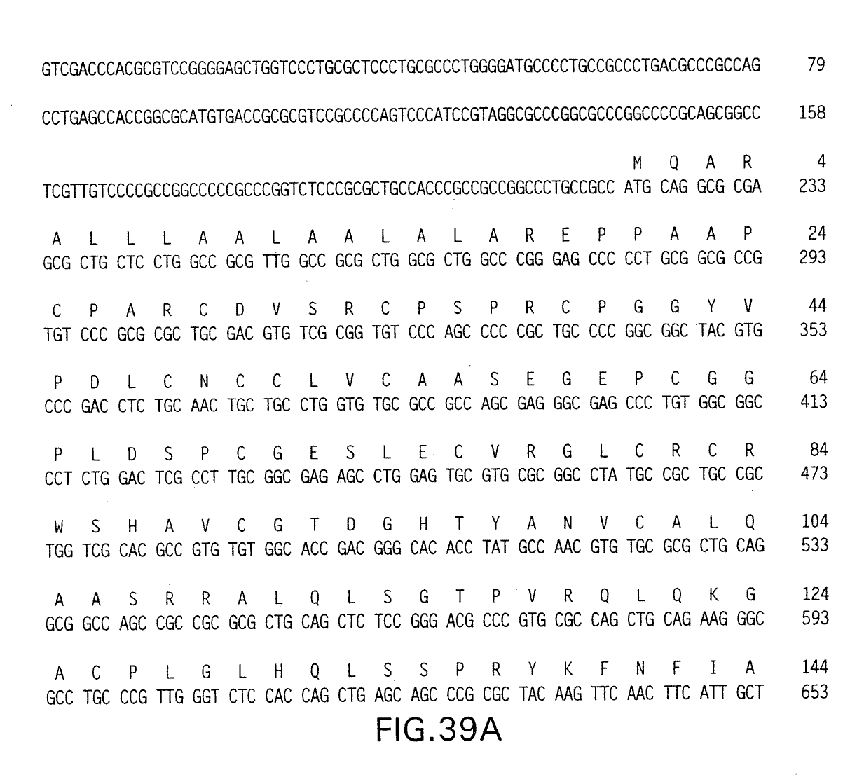

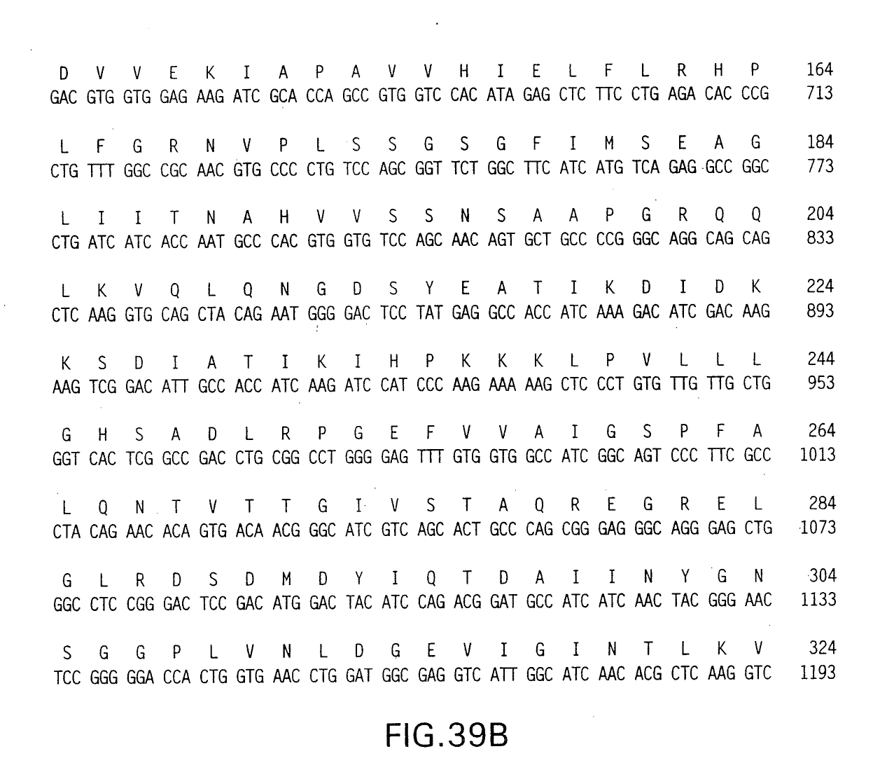

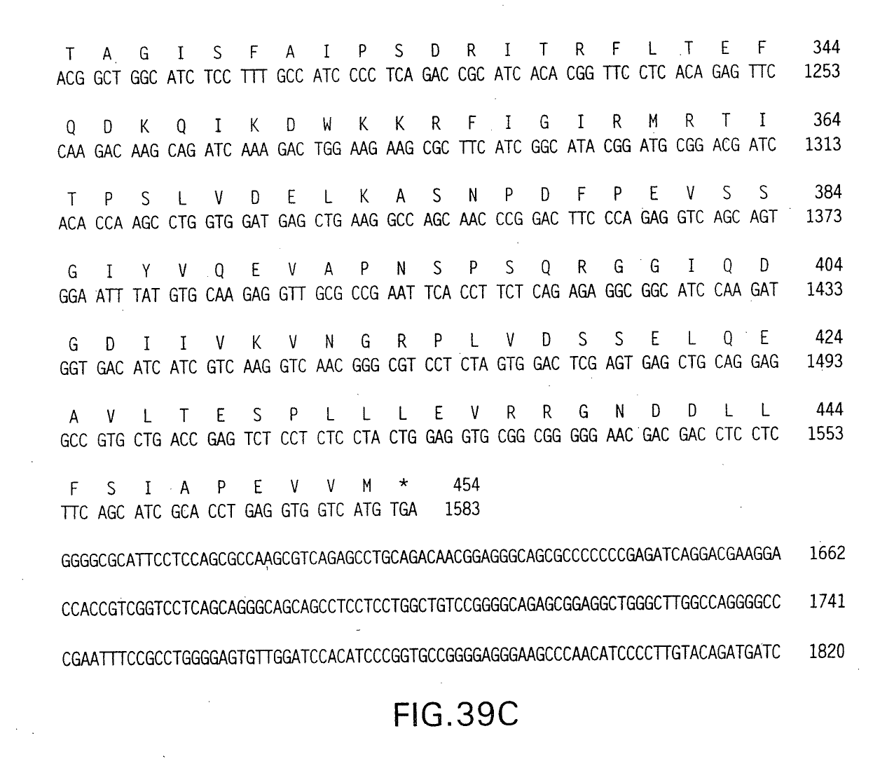

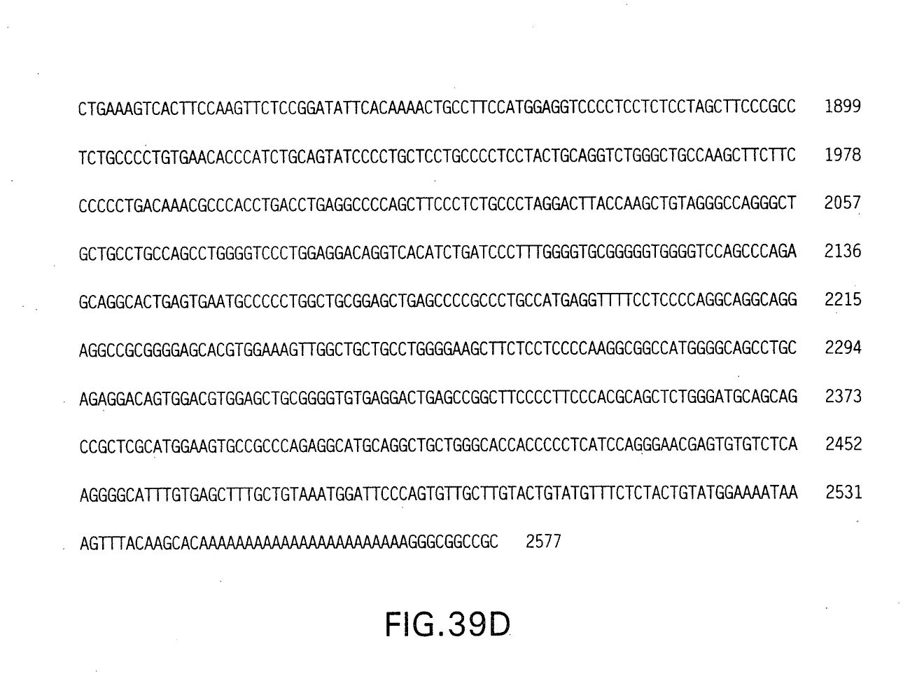

A cDNA encoding human HtrA-2 (TANGO 214) was identified by analyzing the sequences of clones present in an LPS-stimulated osteoblast cDNA library and a prostate stroma cDNA library. This analysis led to the identification of a clone, jthqc058b12, encoding full-length human HtrA-2. The human HtrA-2 cDNA of this clone is 2577 nucleotides long (FIG. 39A-39D; SEQ ID NO:31). The open reading frame of this cDNA (nucleotides 222 to 1580 of SEQ ID NO:31) encodes a 453 amino acid secreted protein (SEQ ID NO:32).

In one embodiment of a nucleotide sequence of human HtrA-2, the nucleotide at position 278 is an guanine (G). In this embodiment, the amino acid at position 19 is glutamate (E). In another embodiment of a nucleotide sequence of human HtrA-2, the nucleotide at position 278 is a cytosine (C). In this embodiment, the amino acid at position 19 is aspartate (D). In another embodiment of a nucleotide sequence of human HtrA-2, the nucleotide at position 395 is guanine (G). In this embodiment, the amino acid at position 58 is glutamate (E). In another embodiment of a nucleotide sequence of human HtrA-2, the nucleotide at position 395 is cytosine (C). In this embodiment, the amino acid at position 58 is aspartate (D). In another embodiment of a nucleotide sequence of human HtrA-2, the nucleotide at position 401 is guanine (G). In this embodiment, the amino acid at position 60 is glutamate (E). In another embodiment of a nucleotide sequence of human HtrA-2, the nucleotide at position 401 is cytosine (C). In this embodiment, the amino acid at position 60 is aspartate (D).

The signal peptide prediction program SIGNALP (Nielsen et al. (1997) Protein Engineering 10:1-6) predicted that human HtrA-2 includes a 17 amino acid signal peptide (amino acid 1 to about amino acid 17 of SEQ ID NO:32) preceding the mature HtrA-2 protein (corresponding to about amino acid 18 to amino acid 453 of SEQ ID NO:32). The HtrA-2 protein molecular weight is 48.6 kDa prior to the cleavage of the signal peptide, 47.0 kDa after cleavage of the signal peptide.

HtrA-2 includes a IGF binding domain (about amino acids 18 to 78 of SEQ ID NO:32), a Kazal protease inhibitor domain (about amino acids 79 to 126 of SEQ ID NO:32), a serine protease domain (about amino acids 140 to 347 of SEQ ID NO:32), and a PDZ domain (about amino acids 348-439 of SEQ ID NO:32).

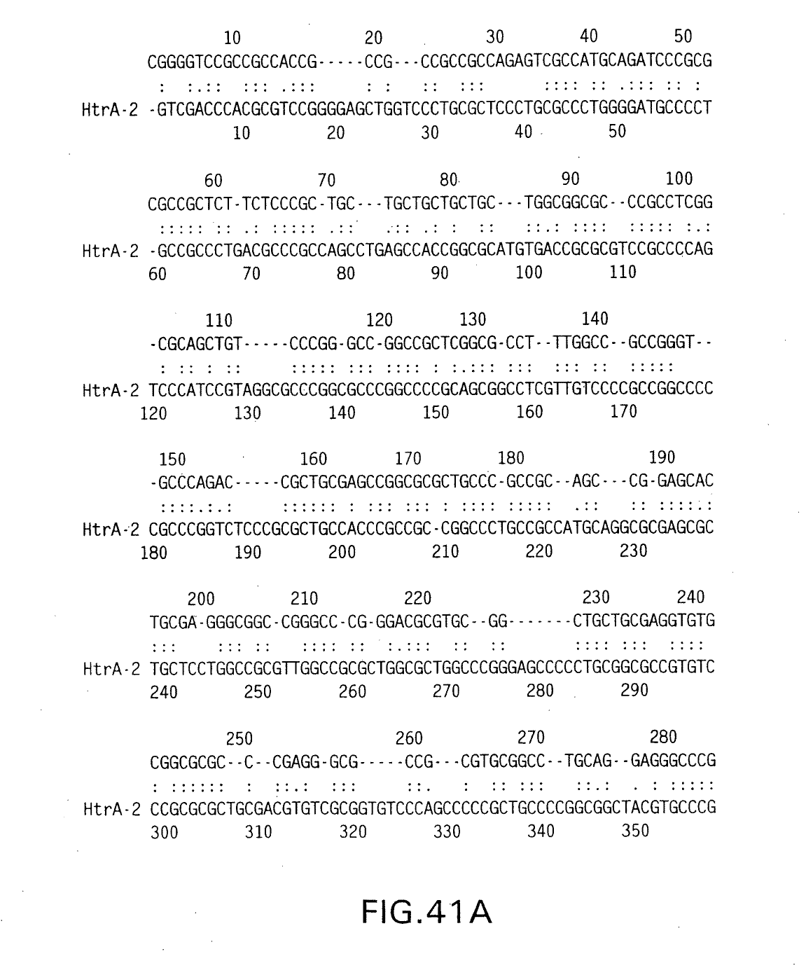

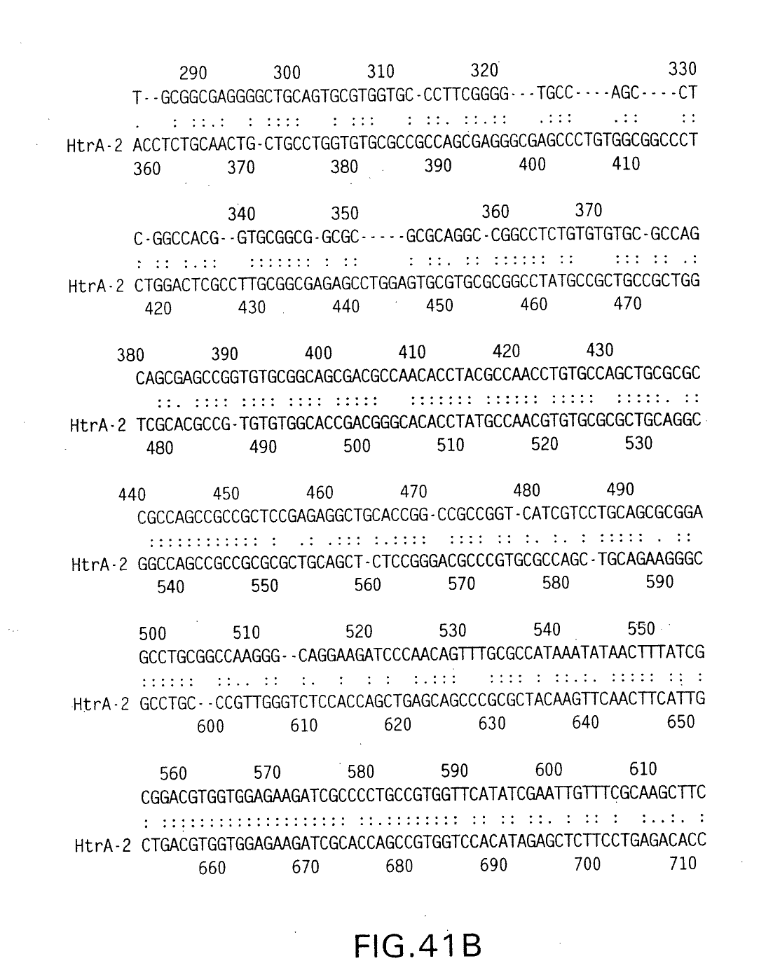

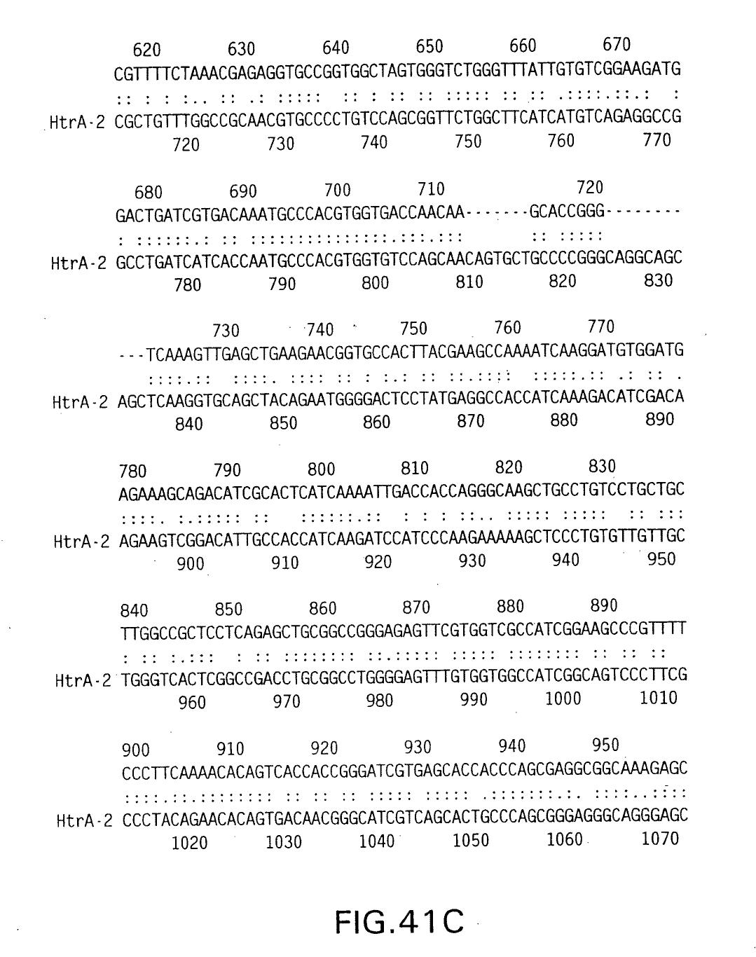

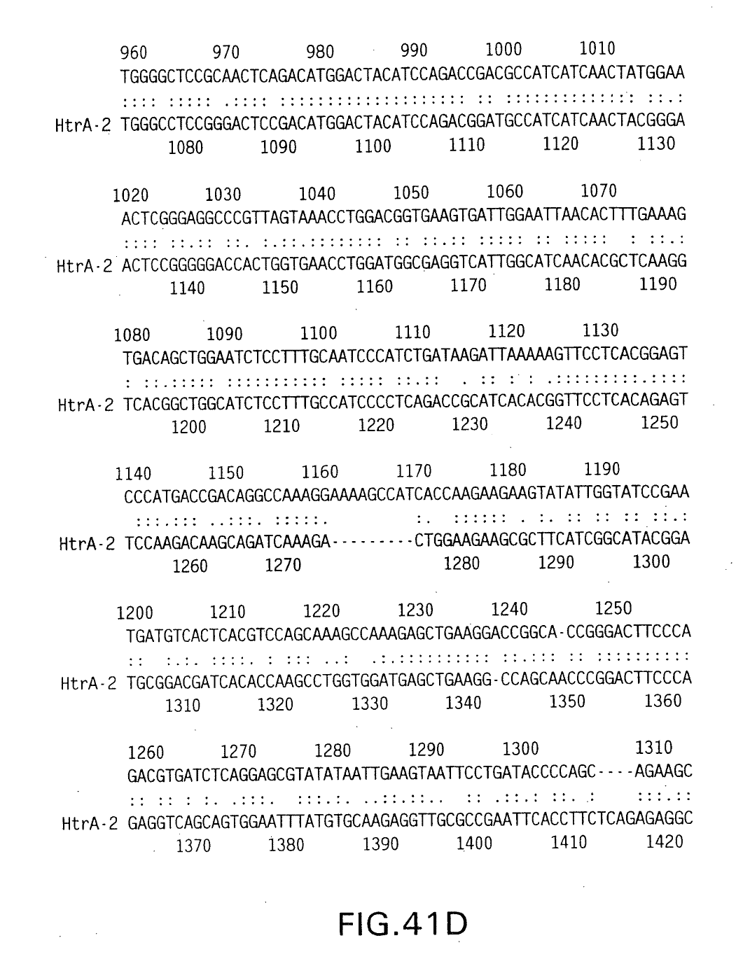

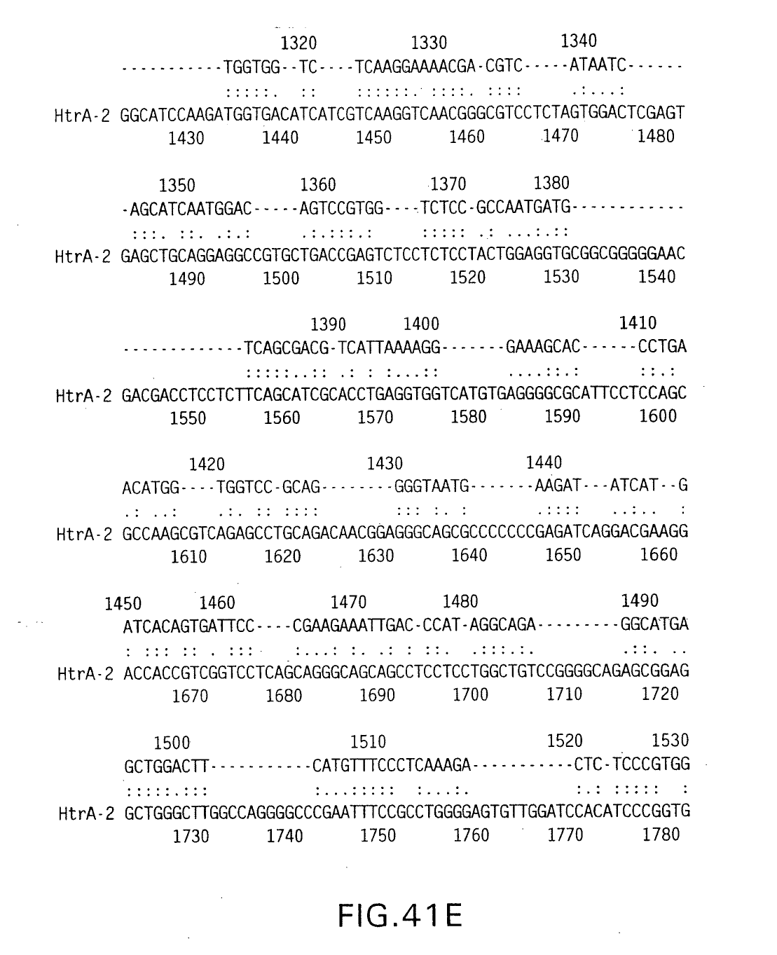

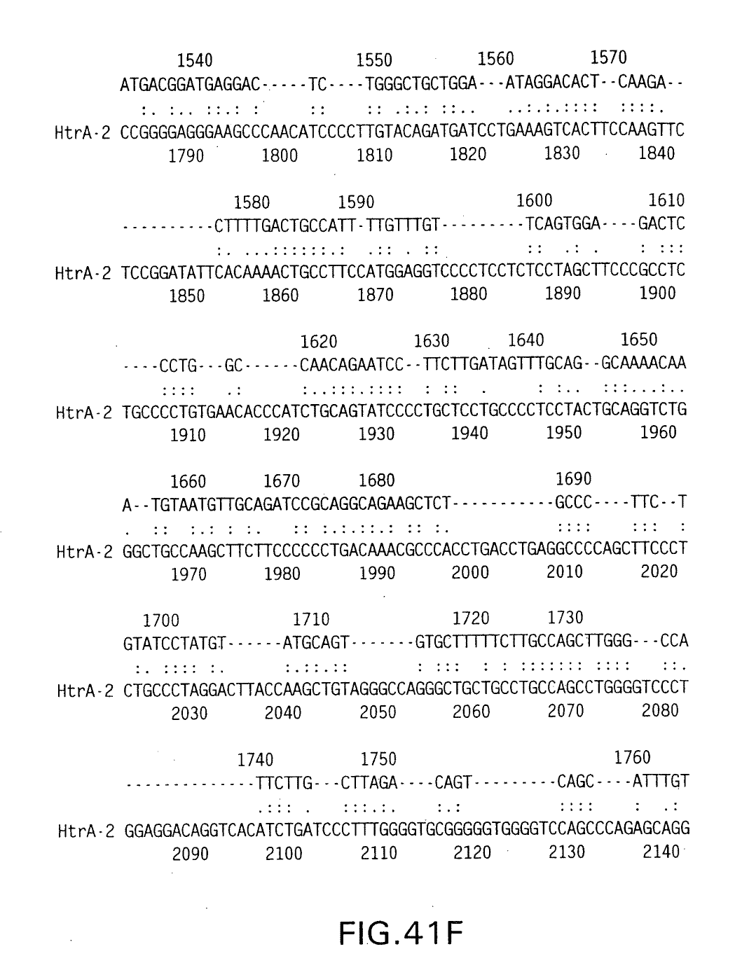

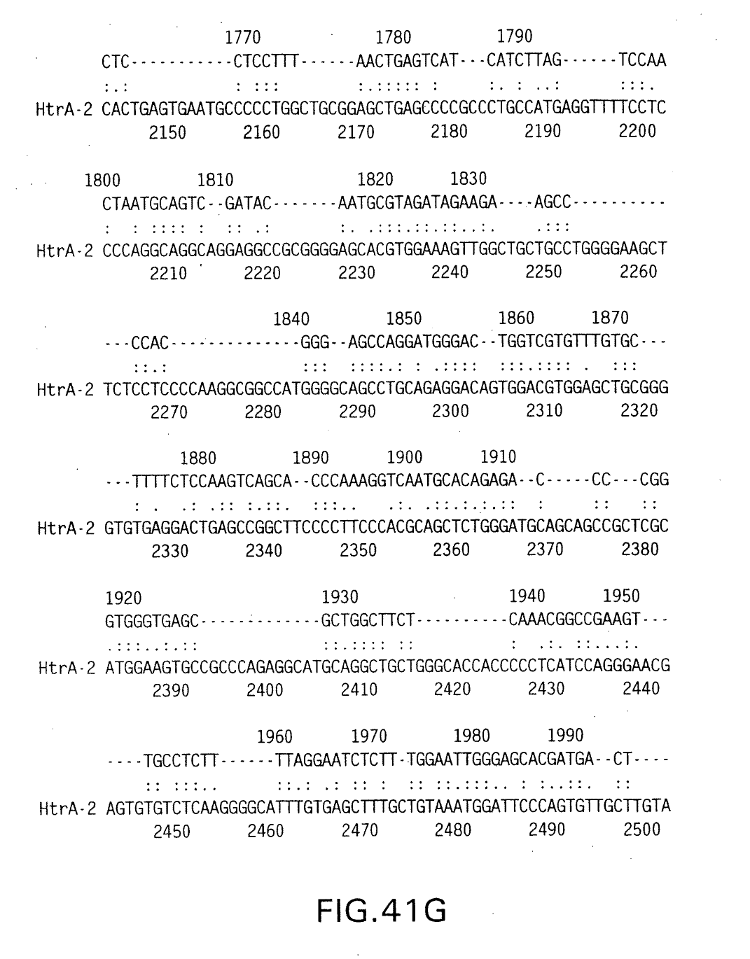

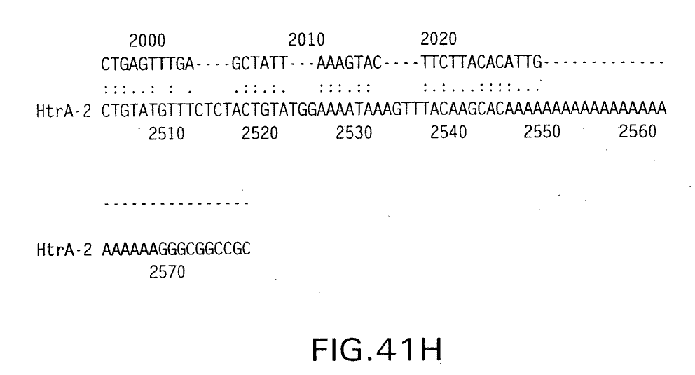

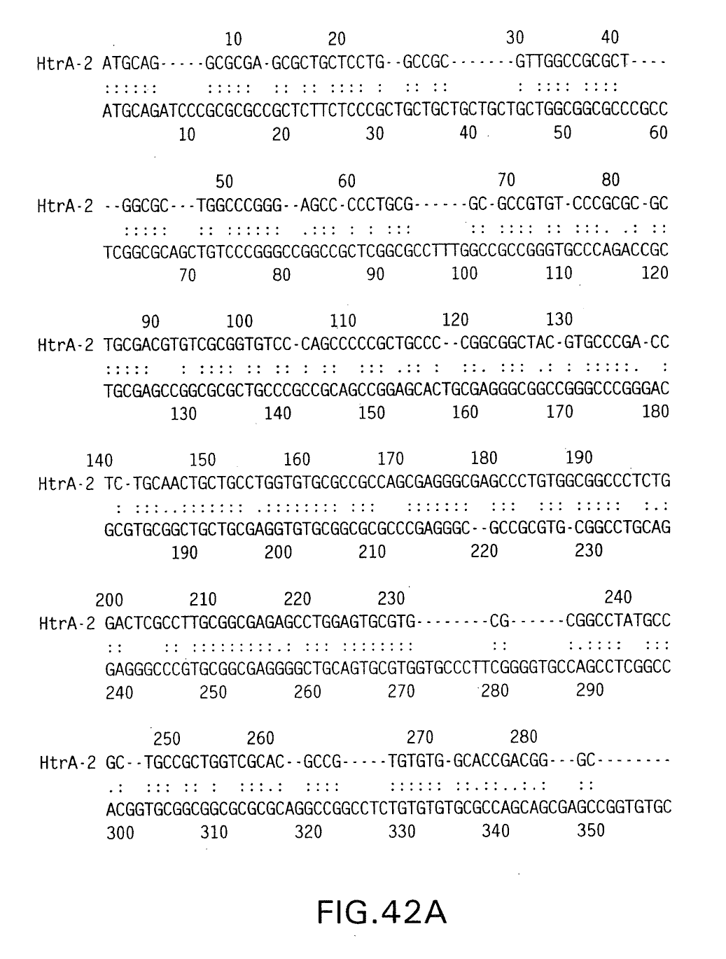

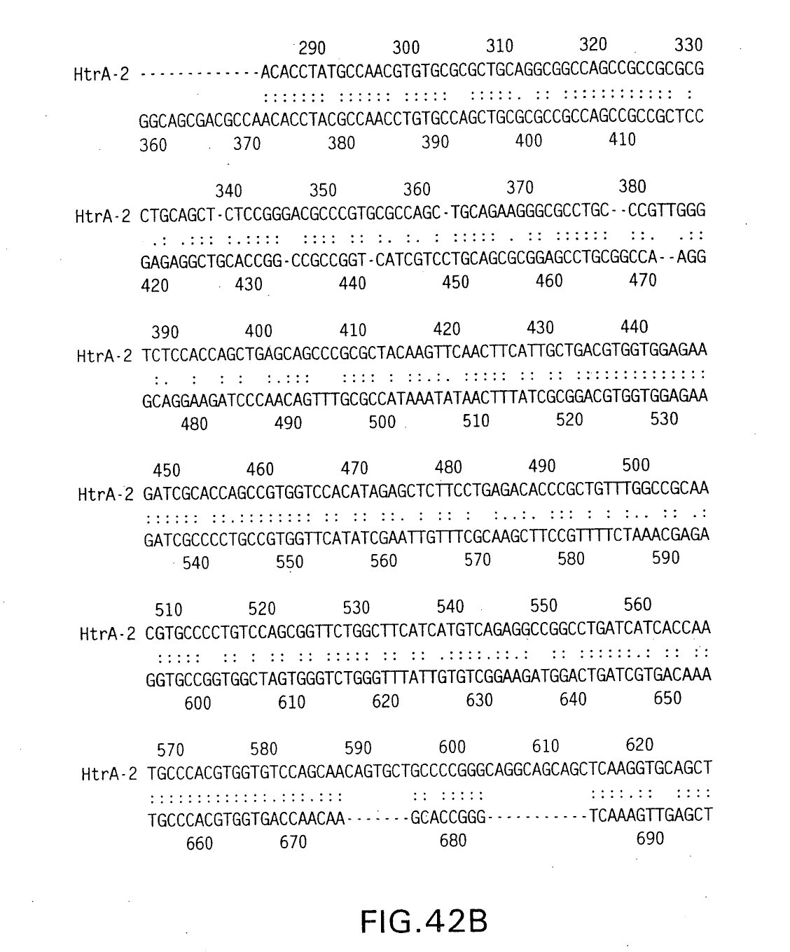

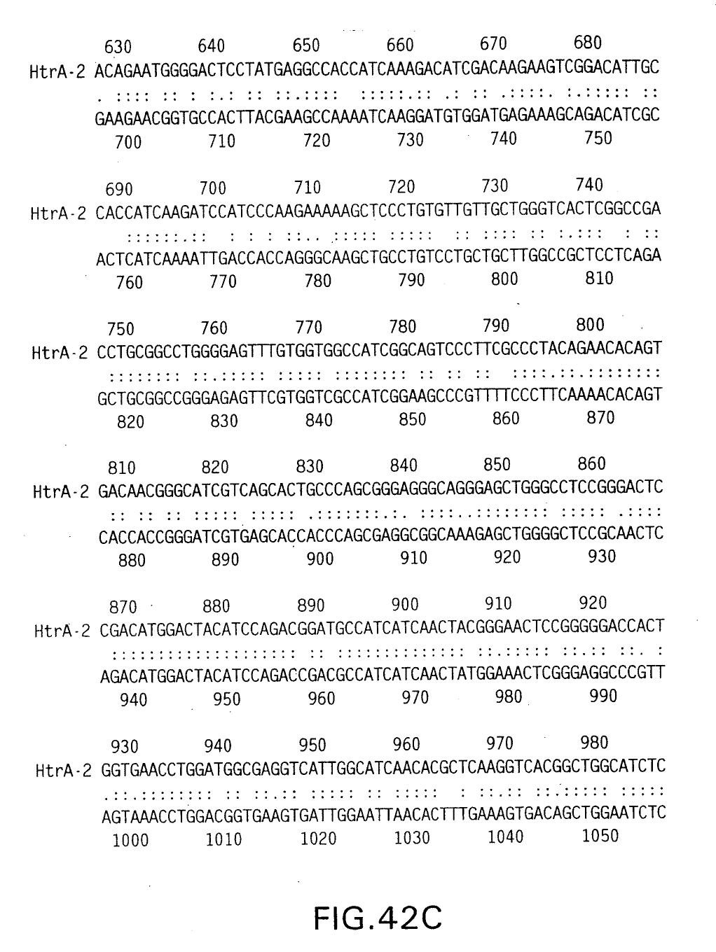

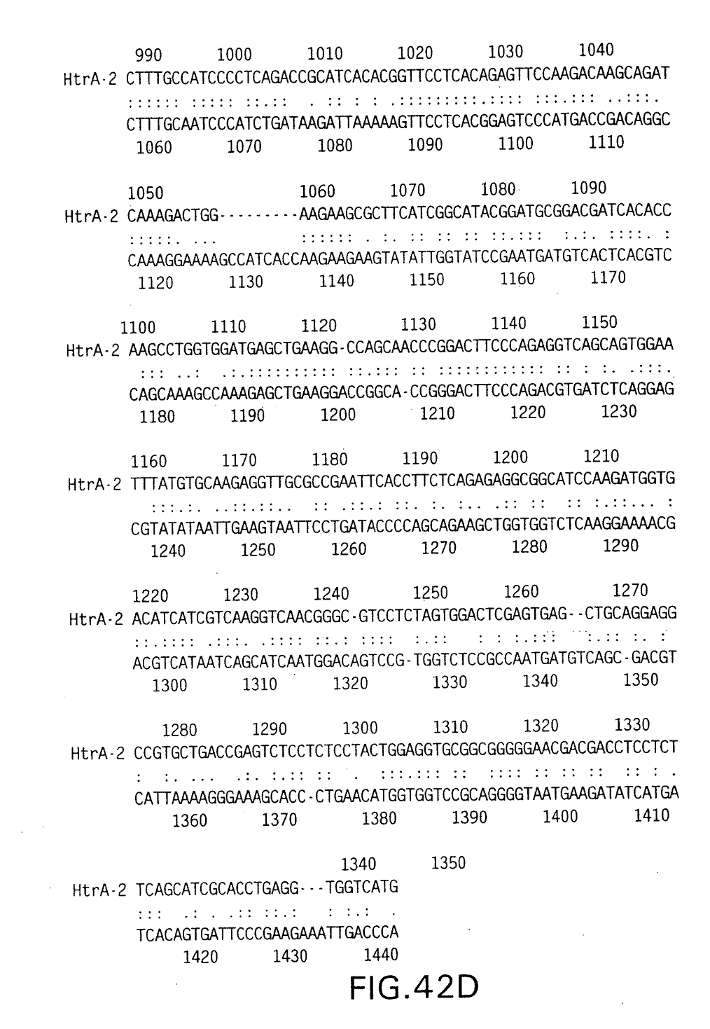

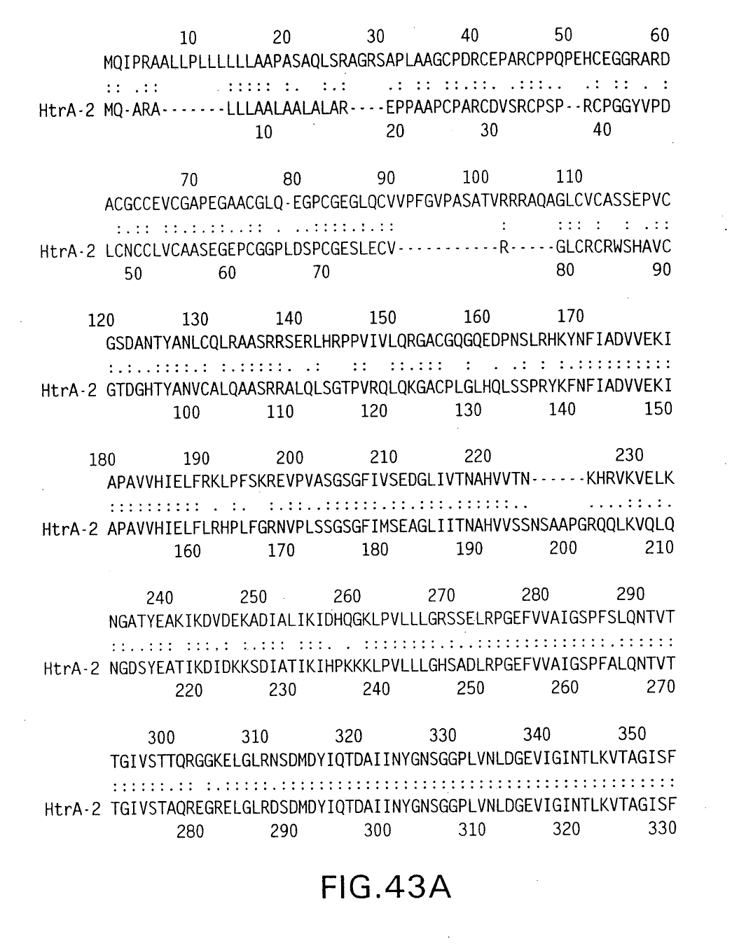

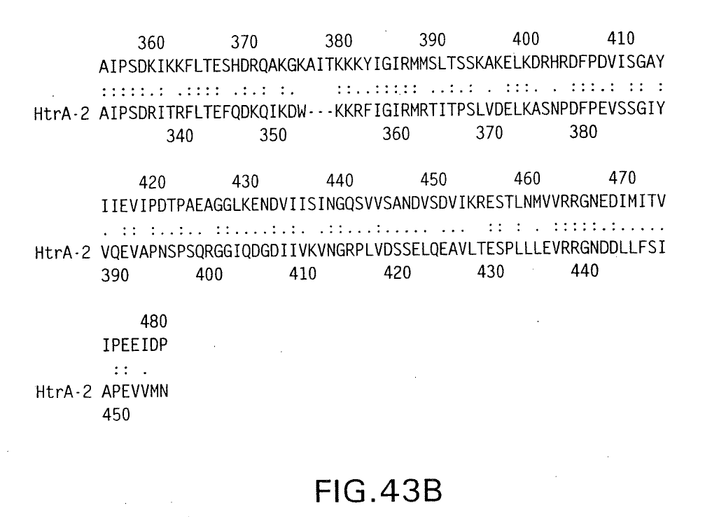

FIG. 41A-41H shows an alignment of the human HtrA-2 full length nucleic acid sequence with the human HtrA full length nucleic acid sequence. FIG. 42A-42D shows an alignment of the human HtrA-2 nucleotide coding region with the human HtrA nucleotide coding region. FIG. 43A-43B shows an alignment of the human HtrA-2 protein sequence with the human HtrA protein sequence. As shown in FIG. 43A-43B, the human HtrA-2 signal sequence is represented by amino acids 1-17 (and encoded by nucleotides 222-272 of SEQ ID NO:31), and the human HtrA signal sequence is represented by amino acids 1-22 (and encoded by nucleotides 39-103 of SEQ ID NO:31). The human HtrA-2 IGF-binding domain sequence is represented by amino acids 18-78 (and encoded by nucleotides 273-455 of SEQ ID NO:31), and the human HtrA IGF-binding sequence is represented by amino acids 37-94 (and encoded by nucleotides 147-320 of SEQ ID NO:31). The human HtrA-2 Kazal protease inhibitor domain sequence is represented by amino acids 79-126 (and encoded by nucleotides 456-599 of SEQ ID NO:31), and the human HtrA Kazal protease inhibitor domain sequence is represented by amino acids 110-155 (and encoded by nucleotides 366-503). The human HtrA-2 serine protease domain sequence is represented by amino acids 140-347 (and encoded by nucleotides 639-1262), and the human HtrA serine protease domain sequence is represented by amino acids 140-369 (and encoded by nucleotides 456-1145). The human HtrA-2 PDZ domain sequence is represented by amino acids 348-439 (and encoded by nucleotides 1263-1538), and the human HtrA PDZ domain sequence is represented by amino acids 370-465 (and encoded by nucleotides 1146-1433).

FIG. 41A-41H and FIG. 42A-42D show that there is an overall 50.9% identity between the full length human HtrA-2 nucleic acid molecule and the full length human HtrA nucleic acid molecule, and an overall 62.3% identity between the open reading frame of human HtrA-2 nucleic acid molecule and the open reading frame of the human HtrA nucleic acid molecule, respectively. The amino acid alignment in FIG. 43A-43B shows a 56.5% overall amino acid sequence identity between human HtrA-2 and human HtrA.

Clone EpT214, which encodes human HtrA-2, was deposited with the American Type Culture Collection (10801 University Boulevard, Manassas, Va. 20110-2209) on Sep. 25, 1998 and assigned Accession Number 98899. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

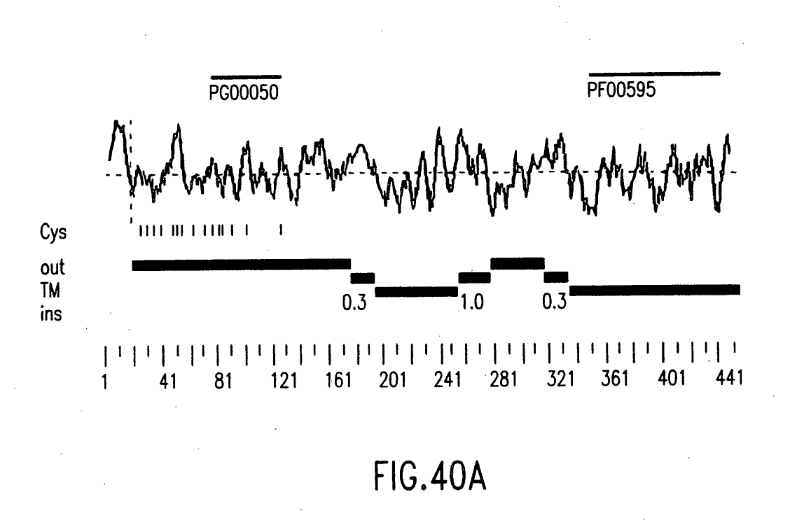

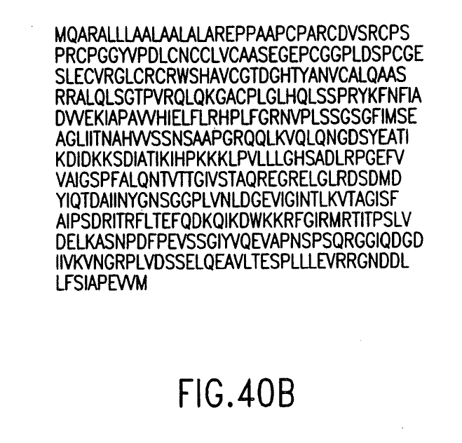

FIG. 40A depicts a hydropathy plot of human HtrA-2.

Northern analysis of HtrA-2 expression in human tissues showed that an approximately 2.6 kB transcript is expressed in human adult heart, skeletal muscle, lung, pancreas, and placenta. No expression was detected in the kidney or brain. In comparison, Northern analysis of HtrA expression in human tissue (Zumbrunn, et al. (1996) FEBS Lett. 398:187-192) indicated that an approximately 2.3 kB transcript is strongly expressed in human placenta, moderately expressed in human brain, liver, and kidney, and weakly expressed in human lung, skeletal muscle, heart, and pancreas.

Library Array Expression: Expression of human HtrA-2 mRNA was detected by a library array procedure. Briefly, this entailed preparing a PCR mixture by adding standard reagents (e.g., Taq Polymerase, dNTPs, and PCR buffer) a vector primer, a primer internal to the gene of interest, and an aliquot of a library in which expression was to be tested. This procedure was performed with many libraries at a time in a 96 well PCR tray, with 80 or more wells containing libraries and a control well in which the above primers were combined with the clone of interest itself. The control well served as an indicator of the fragment size to be expected in the library wells, in the event the clone of interest was expressed within. Amplification was performed in a PCR machine, employing standard PCR conditions for denaturing, annealing, and elongation, and the resultant mixture was mixed with an appropriate loading dye and run on an ethidium bromide-stained agarose gel. The gel was later viewed with UV light after the DNA loaded within its lanes had time to migrate into the gels. Lanes in which a band corresponding with the control band was visible indicated the libraries in which the clone of interest was expressed.

Expression was detected in human umbilical endothelial cells and human lean subcutaneous adipose tissue. No expression was detected in kidney, testes, prostate, HMC-1 control (mast cell line), fetal dorsal spinal cord, human colon to liver metastasis, erythroblasts from CD34+blood, human spinal cord (ION 3), HUVEC TGF-B (human umbilical endothelia), HUVEC (human umbilical endothelia), Brain, K563 (red blood cell line), uterus, Hep-G2 (human insulinoma), human normal colon, human colon to liver metastasis, skin, HUVEC controls (umbilical endothelial cells), human colon (inflammatory bowel disease), melanoma (G361 cell line), adult bone marrow CD34+ cells, HPK, human lung, mammary gland, normal breast epithelium, colon to liver metastasis (CHT128), normal breast, bone marrow (CD34+), W138 (human embryonic lung), Th1 cells, HUVEC untreated (umbilical endothelium), uterus, liver, spleen, normal human Ovarian Epithelia, colon to liver metastasis (CHT133), PTH-treated osteoblasts, ovarian ascites, lung squamous cell carcinoma (MDA 261), Th2 cells, IBD colon (WUM 23), thymus, heart, small intestine, normal megakaryoctyes, colon carcinoma (NDR109), lung adenocarcinoma (PIT245), IBD colon (WUM6), brain-subcortical white matter (ION2), prostate tumor xenograft A12, trigeminal ganglia, 9 week fetus, retinal pigmentosa epithelia, bone marrow, colon carcinoma (NDR103), lung squamous cell carcinoma (PIT299), cervical cancer, normal prostate, prostate tumor, xenograft K10, lumbrosacaral spinal cord, A549 control, stomach, retina, Th-1 induced T cell, colon carcinoma (NDR82), d8 dendritic cells, spinal cord, ovarian epithelial tumor, prostate cancer to liver metastasis JHH3, lumbrosacaral dorsal root ganglia, salivary gland, skeletal muscle, HMC-1 (human mast cell line), Th-2 induced T-cell, colon carcinoma (NDR097), megakaryocytes, dorsal root ganglia (ION 6, 7, 8), HUVEC L-NAME (umbilical endothelia), prostate cancer to liver metastasis JHH4, dorsal root ganglia (ION 6, 7, 8), HMVEC: micro vascular endothelial cells, fetal brain, bronchial epithelium mix, mesangial cells, fetal heart, LPS-stimulated 24 hours Osteoblasts, cervical carcinoma A2780 WT cell line, UCLA-lung carcinoma R (carcinoma (Resistant to drug treatment)), erythroleukemia cells, trachea, testes, placenta, HUVEC: umbilical vein endothelial cells, bronchial epithelium, congestive heart failure, bladder carcinoma T24 cell line Ctl., mammary gland, burkitt's lymphoma, cervical carcinoma A2780 ADR cell line (drug resistant), UCLA-lung carcinoma S (carcinoma (sensitive to drug treatment)), embryonic keratinocytes, cervix carcinoma ME180 IL-1, testes, mammary gland, HL60/S, astrocytes, cerebellum, bladder carcinoma T24 Tr., natural killer cells, fetal spleen, Prostate, fetal fibroblast, SCC25 CDDP—tongue squamous carcinoma, cervix carcinoma, ME 180 control, RAJI-human burkitt's lymphoma B cell, small intestine, U937/A10P10, prostate epithelium, pituitary, prostate fibroblast, congestive heart failure, uterine smooth Muscle, treated, esophagus, p65 IL-1, SCC25 WT-tongue squamous cell carcinoma, MCP-1 mast cell line, ST486 (Lymphoma B cell), fetal liver, U937/A10p50, primary osteoblast, Aortic endothelial cells, bone marrow, prostate smooth muscle, umbilical smooth muscle, treated, fetal liver, lung carcinoma A549 control, fetal hypothalamus, HPK II keratinocyte cell line, HL60 (acute Promyelocytic Leukemia), skeletal muscle, CaCo, keratinocytes, fetal Kidney, congestive heart failure, thyroid, bronchial smooth muscle, fetal skin, A549IL-1, T cells, CD3 treated, lung, umbilical smooth muscle, treated, stomach, HeLa cells, melanocytes, fetal liver, adrenal gland, LPS-stimulated osteoblasts, 1 hour, WT LNCap+casodex, fetal adrenal gland, fetal testes, T cells, CD3 IL4/IL-10 treated, heart, uterine smooth muscle, spleen, HL60/Adr, coronary smooth muscle cells, fetal lung, fetal thymus, LPS-stimulated 6 hour osteoblasts, WT LNCap+Testosterone, midterm placenta, pulmonary artery smooth muscle, T cells, CD3 IFN-γ/TFN-a treated, fetal brain, and liver.

### MOUSE HtrA-2

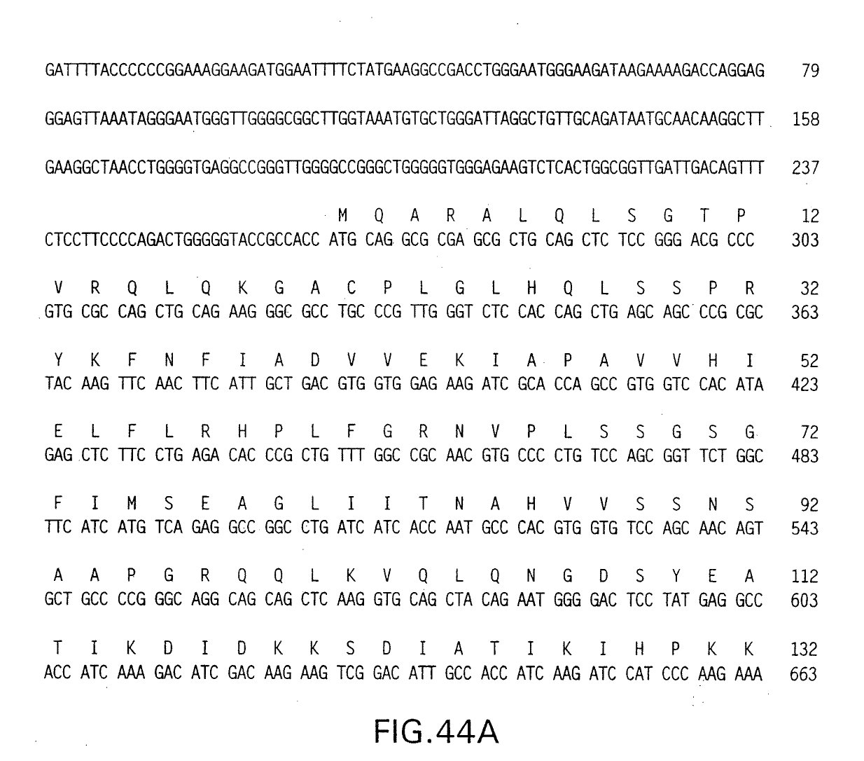

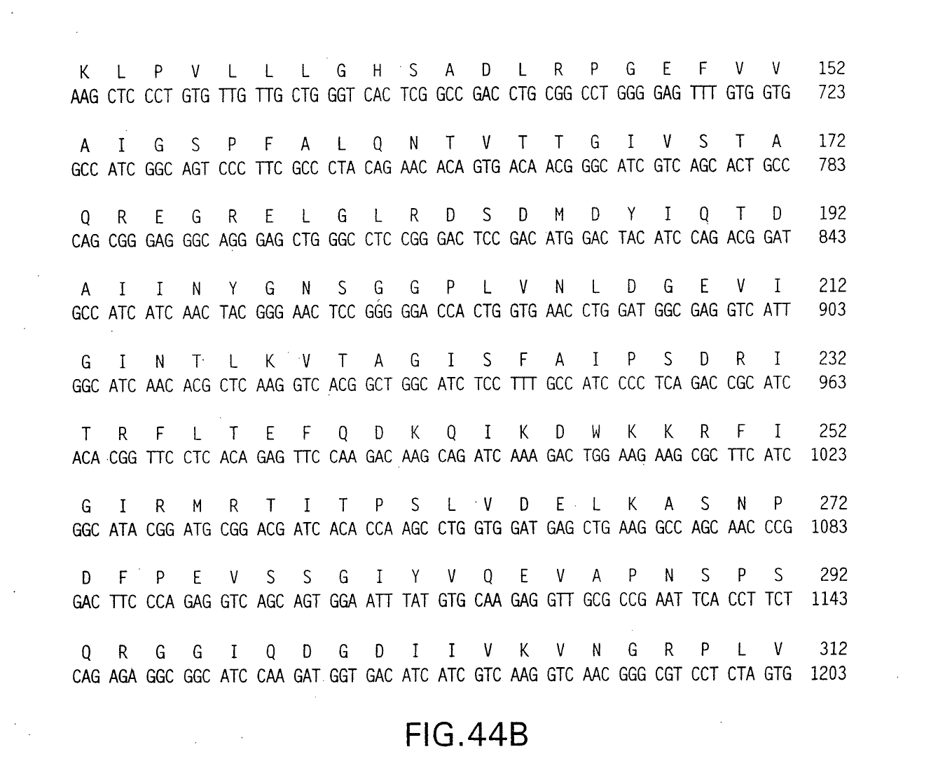

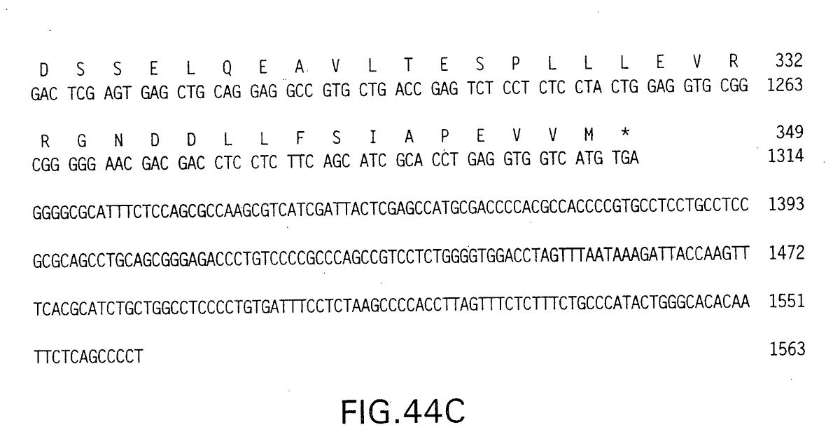

A mouse homolog of human HtrA-2 was identified. A cDNA encoding mouse HtrA-2 was identified by analyzing the sequences of clones present in a mouse cDNA library. This analysis led to the identification of a clone, Atm×2143, encoding full-length mouse HtrA-2. The mouse HtrA-2 cDNA of this clone is 1563 nucleotides long (FIG. 44A-44C; SEQ ID NO:33). The open reading frame of this cDNA (nucleotides 268 to 1311 of SEQ ID NO:33) and encodes a 349 amino acid secreted protein (SEQ ID NO:34).

In one embodiment of a nucleotide sequence of mouse HtrA-2, the nucleotide at position 396 is an guanine (G). In this embodiment, the amino acid at position 43 is glutamate (E). In another embodiment of a nucleotide sequence of mouse HtrA-2, the nucleotide at position 396 is a cytosine (C). In this embodiment, the amino acid at position 43 is aspartate (D). In another embodiment of a nucleotide sequence of mouse HtrA-2, the nucleotide at position 426 is guanine (G). In this embodiment, the amino acid at position 53 is glutamate (E). In another embodiment of a nucleotide sequence of mouse HtrA-2, the nucleotide at position 426 is cytosine (C). In this embodiment, the amino acid at position 53 is aspartate (D). In another embodiment of a nucleotide sequence of mouse HtrA-2, the nucleotide at position 498 is guanine (G). In this embodiment, the amino acid at position 77 is glutamate (E). In another embodiment of a nucleotide sequence of mouse HtrA-2, the nucleotide at position 498 is cytosine (C). In this embodiment, the amino acid at position 77 is aspartate (D).

HtrA-2 mRNA expression in mouse: In situ tissue screening was performed on mouse adult and embryonic tissue to analyze for the expression of mouse HtrA-2 mRNA. In summary, adult expression was highest in the bladder and present to a lesser extent in heart, muscle, and colon. Signal in these tissues was ubiquitous. All other adult tissues showed no specific signal above background. Expression at embryonic day 13.5, the earliest age tested, was observed in the stomach and brain. Expression pattern in brain was punctate, broadly distributed, and sparse. Beginning at E14.5 ubiquitous expression was also observed in skeletal muscle, diaphragm, intestine, and lung. This pattern continues until postnatal day 1.5, when expression was also apparent in the renal medulla. There was high background in what appears to be cartilage. The antisense probe showed a stronger signal in this tissue with a more extensive pattern. In particular, with respect to adult mouse expression, expression was ubiquitous in each of the bladder, heart, skeletal muscle, and colon. No expression was detected in the following tissues: lung, brain, placenta, liver, pancreas, thymus, eye, kidney, and the small intestine. With respect to expression in the embryonic mouse, the following results were obtained: At E13.5, expression was detected in the stomach and brain. Signal was also observed in the limbs and vertebrae with the sense probe. This signal was much higher and more extensive with the antisense probe. At E14.5, E15.5, E16.5, E18.5, and P1.5, a signal was punctuate in the brain, strong in renal medulla and absent from liver. Most other tissues had low level ubiquitous expression to some degree.

### Uses of HtrA-2 (TANGO 214) Nucleic Acids, Polypeptides, and Modulators Thereof

As HtrA-2 was originally found in an LPS-treated osteoblast library and is homologous to HtrA, mRNA levels of which are known to be elevated in cartilage from individuals with osteoarthritis, HtrA-2 nucleic acids, proteins, and modulators thereof can be used to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

As HtrA-2, like HtrA, is highly expressed in the heart, and includes an IGF-binding domain, and thus likely has a role in modulating IGF function (e.g., IGF is involved in cardiac hyperplasia), HtrA-2 nucleic acids, proteins, and modulators thereof can be used to treat disorders of the cardiovascular system. Examples of disorders of the cardiovascular system include various forms of heart disease include but are not limited to: aortic valve prolapse; aortic valve stenosis; arrhythmia; cardiogenic shock; heart attack; heart failure; heart tumor; heart valve pulmonary stenosis; mitral regurgitation (acute); mitral regurgitation (chronic); mitral stenosis; mitral valve prolapse; stable angina; tricuspid regurgitation, angina pectoris, myocardial infarction, and chronic ischemic heart disease, hypertensive heart disease, pulmonary heart disease, valvular heart disease (e.g., rheumatic fever and rheumatic heart disease, endocarditis, mitral valve prolapse, and aortic valve stenosis), congenital heart disease (e.g. valvular and vascular obstructive lesions, atrial or ventricular septal defect, and patent ductus arteriosus), or myocardial disease (e.g., myocarditis, congestive cardiomyopathy, and hypertrophic cardiomyopathy). Disorders of the vasculature that can be treated or prevented according to the methods of the invention include atheroma, tumor angiogenesis, wound healing, diabetic retinopathy, hemangioma, psoriasis, and restenosis, e.g., restenosis resulting from balloon angioplasty.

More particularly, HtrA-2 nucleic acids, proteins, and modulators thereof can be used to treat congestive heart failure may affect either the right side, left side, or both sides of the heart. Further, HtrA-2 nucleic acids, proteins, and modulators thereof can be used to treat structural or functional causes of heart failure include high blood pressure (hypertension), heart valve disease, and other heart diseases.

HtrA-2 nucleic acids, proteins, and modulators thereof can also be used to treat cardiomyopathy. Specific types of cardiomyopathy include: ischemic cardiomyopathy; idiopathic cardiomyopathy; hypertrophic cardiomyopathy; alcoholic cardiomyopathy; peripartum cardiomyopathy; dilated cardiomyopathy; and restrictive cardiomyopathy.

The presence of an IGF binding domain in HtrA-2 also suggests that HtrA-2 can modulate IGF function and thereby be used to treat IGF associated disorders. IGFs are known to be involved in the overall cellular growth of embryos and organs of mammals. When existing at excessive levels, however, IGFs can cause somatic overgrowth which leads to conditions such as visceromegaly, placentomegaly, cardiac and adrenal defects, and Beckwith-Weidermann syndrome. Thus, HtrA-2 nucleic acids, proteins, and modulators thereof can be used to treat IGF-associated disorders as described above. In addition, as IGF can cause increased cell proliferation, HtrA-2 nucleic acids, proteins, and modulators thereof can be used to treat proliferative disorders, e.g., cancer, e.g., cancer of a cell or tissue in which HtrA-2 is expressed.

The presence of a Kazal protease inhibitor domain in HtrA-2 also indicates that HtrA-2 can function in a similar manner as other proteins containing a Kazal protease inhibitor domain. For example, follistatin includes a Kazal protease inhibitor domain. Follistatin regulates the availability of growth factors and embryonic growth, and thus modulators thereof can be used to treat disorders involving abnormal cellular migration, proliferation, and differentiation. Similarly, HtrA-2 nucleic acids, proteins, and modulators thereof can be used to treat disorders involving abnormal cellular migration, proliferation (e.g., cancer), and/or differentiation, and/or follistatin-associated disorders.

As HtrA-2 includes a serine protease domain, it can act as a serine protease. Thus, HtrA-2 nucleic acids, proteins, and modulators thereof can be used to treat disorders involving abnormal serine protease function. For example, it is known that serine protease inhibitors are abundant in plaques found in Alzheimer's patients, and may be responsible for preventing some types of metalloproteinase from breaking down the beta-amyloid proteins that make up these plaques. Thus, modulation of the HtrA-2 serine protease activity may modulate formation of Alzheimer's plaques. Consequently, HtrA-2 nucleic acids, proteins, and modulators thereof can be used to treat Alzheimer's disease.

The presence of a PDZ domain in HtrA-2 suggests that HtrA-2 functions in a manner similar to other PDZ-containing proteins. For example, PDZ domains typically bind other proteins at their carboxyl termini in a sequence-specific manner.

Human HtrA-2 nucleic acids, proteins, and modulators thereof can also be used to treat neurological disorders. Examples of such neurological disorders include disorders due to nerve damage (e.g., nerve damage due to stroke) and neurodegenerative diseases (e.g., Alzheimer's disease, multiple sclerosis, Huntington's disease, and Parkinson's disease). In addition, human HtrA-2 nucleic acids, polypeptides, and modulators thereof can be used to treat neurodegeneration associated with Alzheimer's disease, frontal lobe dementia, cortical lewy body disease, dementia of Parkinson's disease, acute and chronic phases of degeneration following stroke or head injury, neuronal degeneration found in motor neuron disease, AIDS dementia and chronic epilepsy.

HtrA-2, like HtrA, can likely interact with a normal or mutated gene product of a human presenilin gene (e.g., human presenilin-1 (PS-1), e.g., the hydrophobic loop domain between transmembrane domains 1 and 2 of PS-I). As mutations in the human PS-I gene lead to Familial Alzheimer's disease (see PCT Publication Number WO98/01549, the contents of which are incorporated by reference), and HtrA-2 can interact with PS-I and thus modulate PS-I function, HtrA-2 nucleic acids, proteins, and modulators thereof can be used to treat Alzheimer's disease and physiological functions associated with Alzheimer's disease.

The PS-1 gene product may also be a receptor or channel protein, mutations in which have been causally related to neurological disorders whose pathology does not represent Alzheimer's disease. Thus, HtrA-2 nucleic acids, proteins, and modulators thereof can be used to treat non-Alzheimer's neurological disorders as well (e.g., malignant hyperthermia, hyperkalemic periodic paralysis).

HtrA-2 nucleic acids, proteins, and modulators thereof can also be used to treat disorders of the cells and tissues in which it is expressed. As HtrA-2 is expressed in colon, bladder, skeletal muscle, lung, pancreas, and placenta, HtrA-2 nucleic acids, proteins, and modulators thereof can be used to treat disorders of these cells, tissues, or organs, e.g., colon cancer and colonic volvulus, diverticula, cystitis, urinary tract infection, bladder cancer, muscular dystrophy, stroke, muscular atrophy, trichinosis, lung cancer, cystic fibrosis, rheumatoid lung disease, pancreatic cancer, diabetes, pancreatitis, and various placental disorders.

Because HtrA-2 was expressed in adipose tissue, HtrA-2 nucleic acids, proteins and modulators thereof can be utilized to modulate adipocyte function and adipocyte-related processes and disorders such as, e.g., obesity, regulation of body temperature, lipid metabolism, carbohydrate metabolism, body weight regulation, obesity, anorexia nervosa, diabetes mellitus, unusual susceptibility or insensitivity to heat or cold, arteriosclerosis, atherosclerosis, and disorders involving abnormal vascularization, e.g., vascularization of solid tumors. Additionally, such molecules can be used to treat disorders associated with abnormal fat metabolism, e.g., cachexia. In another example, such molecules can be used to treat disorders associated with abnormal proliferation of these tissues, e.g., cancer, e.g., breast cancer or liver cancer.

### Human TANGO 221

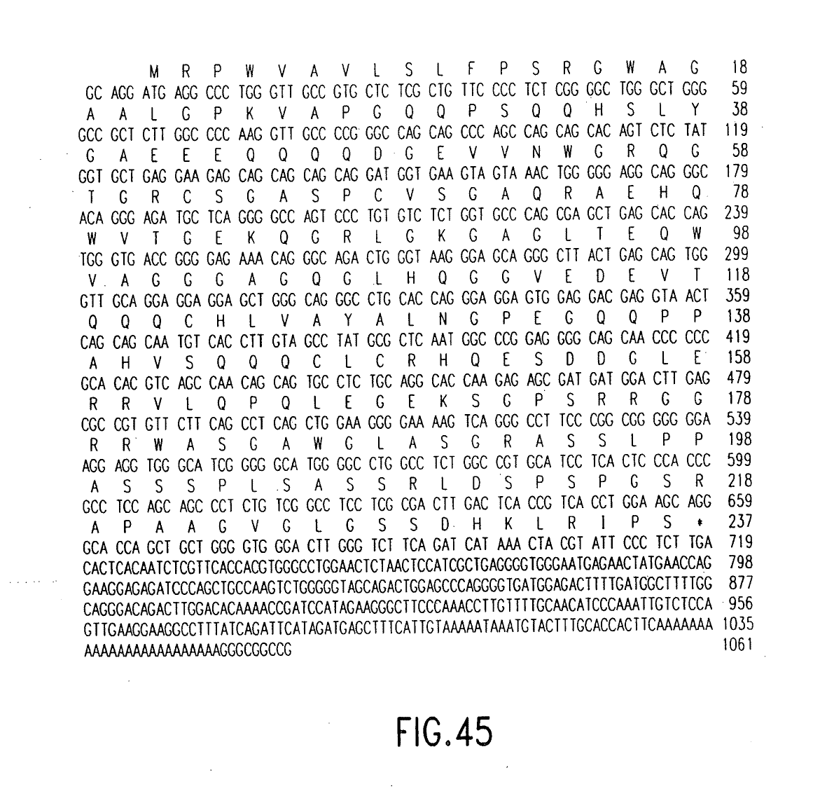

A cDNA encoding TANGO 221 was identified by analyzing the sequences of clones present in a non-obese human subcutaneous adipose tissue cDNA library. This analysis led to the identification of a clone, Athfa28c12, encoding full-length TANGO 221. The cDNA of this clone is 1061 nucleotides long (FIG. 45; SEQ ID NO:35). It is noted that the nucleotide sequence contains a Not I adapter sequence on the 3′ end. The open reading frame of this cDNA, nucleotides 6 to 716, encodes a 237 amino acid secreted protein (SEQ ID NO:36).

In one embodiment of a nucleotide sequence of human TANGO 221, the nucleotide at position 128 is a guanine (G). In this embodiment, the amino acid at position 41 is glutamate (E). In another embodiment of a nucleotide sequence of human TANGO 221, the nucleotide at position 128 is a cytosine (C). In this embodiment, the amino acid at position is 41 aspartate (D). In another embodiment of a nucleotide sequence of human TANGO 221, the nucleotide at position 131 is adenine (A). In this embodiment, the amino acid at position 42 is glutamate (E). In another embodiment of a nucleotide sequence of human TANGO 221, the nucleotide at position 131 is cytosine (C). In this embodiment, the amino acid at position 42 is aspartate (D). In another embodiment of a nucleotide sequence of human TANGO 221, the nucleotide at position 134 is guanine (G). In this embodiment, the amino acid at position 43 is glutamate (E). In another embodiment of a nucleotide sequence of human TANGO 221, the nucleotide at position 134 is cytosine (C). In this embodiment, the amino acid at position 43 is aspartate (D).

The signal peptide prediction program SIGNALP (Nielsen et al. (1997) Protein Engineering 10:1-6) predicted that TANGO 221 includes an 17 amino acid signal peptide (amino acid 1 to about amino acid 17 of SEQ ID NO:36) preceding the mature TANGO 221 protein (corresponding to about amino acid 18 to amino acid 237 of SEQ ID NO:36). TANGO 221 is predicted to have a molecular weight of 24.7 kDa prior to cleavage of its signal peptide and a molecular weight of 22.8 kDa subsequent to cleavage of its signal peptide.

In certain embodiments, a TANGO 221 family member has the amino acid sequence of SEQ ID NO:36, and the signal sequence is located at amino acids 1 to 15, 1 to 16, 1 to 17, 1 to 18, or 1 to 19. In such embodiments of the invention, the domains and the mature protein resulting from cleavage of such signal peptides are also included herein. For example, the cleavage of a signal sequence consisting of amino acids 1-17, results in a mature TANGO 221 protein corresponding to amino acids 18 to 237. The signal sequence is normally cleaved during processing of the mature protein.

A casein kinase II phosphorylation site having the sequence SRLD is found from amino acids 208 to 211. A protein kinase C phosphorylation site having the sequence TGR is found from amino acids 59 to 61. A second protein kinase C phosphorylation site having the sequence SRR is found from amino acids 174 to 176. A third protein kinase C phosphorylation site having the sequence SGR is found from amino acids 190 to 192. A fourth protein kinase C phosphorylation site having the sequence SSR is found from amino acids 207 to 209. An N-myristoylation site having the sequence GQQPSQ is found from amino acids 28 to 33. A second N-myristoylation site having the sequence GTGRCS is found from amino acids 58 to 63. A third second N-myristoylation site having the sequence GASPCV is found from amino acids 64 to 69. A fourth N-myristoylation site having the sequence GAQRAE is found from amino acids 71 to 76. A fifth N-myristoylation site having the sequence GAGLTE is found from amino acids 91 to 96. A sixth N-myristoylation site having the sequence GGGAGQ is found from amino acids 101 to 106. A seventh N-myristoylation site having the sequence GLHQGG is found from amino acids 107 to 112. An eighth N-myristoylation site having the sequence GLASGR is found from amino acids 187 to 192. A ninth N-myristoylation site having the sequence GVGLGS is found from amino acids 223 to 228. An amidation site having the sequence GGRR is found from amino acids 177 to 180.

A clone EpT221, which encodes human TANGO 221, was deposited with the American Type Culture Collection (10801 University Boulevard, Manassas, Va. 20110-2209) on Jan. 7, 1999 and assigned Accession Number 207044. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

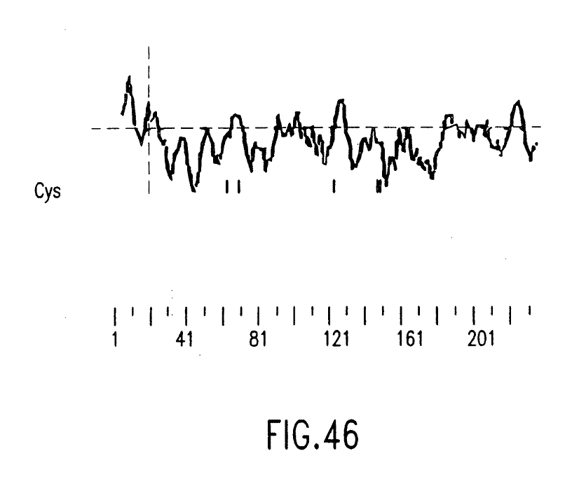

FIG. 46 depicts a hydropathy plot of human TANGO 221. The dashed vertical line separates the signal sequence (amino acids 1-17 of SEQ ID NO:36) on the left from the mature protein (amino acids 18-237 of SEQ ID NO:36) on the right.

### Uses of TANGO 221 Nucleic Acids, Polypeptides, and Modulators Thereof

Because TANGO 221 is expressed in cells of subcutaneous adipose tissue, breast tissue, and fetal liver and spleen tissue, TANGO 221 polypeptides, nucleic acids, and modulators thereof, can be used to modulate the function, morphology, proliferation and/or differentiation of cells in the tissues in which it is expressed. For example, TANGO 221 nucleic acids, proteins and modulators thereof can be utilized to modulate adipocyte function and adipocyte-related processes and disorders such as, e.g., obesity, regulation of body temperature, lipid metabolism, carbohydrate metabolism, body weight regulation, obesity, anorexia nervosa, diabetes mellitus, unusual susceptibility or insensitivity to heat or cold, arteriosclerosis, atherosclerosis, and disorders involving abnormal vascularization, e.g., vascularization of solid tumors. Additionally, such molecules can be used to treat disorders associated with abnormal fat metabolism, e.g., cachexia. In another example, such molecules can be used to treat disorders associated with abnormal proliferation of these tissues, e.g., cancer, e.g., breast cancer or liver cancer.

As TANGO 221 exhibits expression in the spleen, TANGO 221 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 221 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 221 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

In another example, because TANGO 221 exhibits expression in the liver, TANGO 221 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

### Human TANGO 222

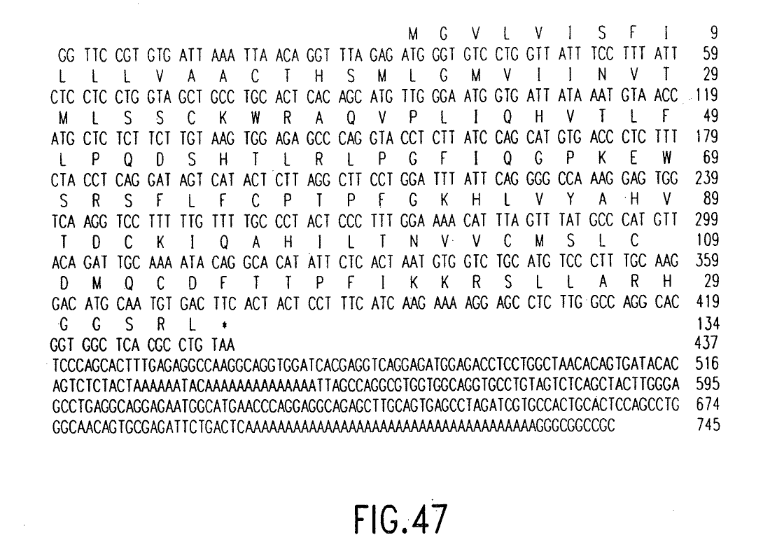

A cDNA encoding TANGO 222 was identified by analyzing the sequences of clones present in a non-obese human subcutaneous adipose tissue cDNA library. This analysis led to the identification of a clone, Athfa59d4, encoding full-length TANGO 222. The cDNA of this clone is 745 nucleotides long (FIG. 47; SEQ ID NO:37). The open reading frame of this cDNA, nucleotides 33 to 434 of SEQ ID NO:38), encodes a 134 amino acid secreted protein (SEQ ID NO:38).

In one embodiment of a nucleotide sequence of human TANGO 222, the nucleotide at position 236 is a guanine (G). In this embodiment, the amino acid at position 68 is glutamate (E). In another embodiment of a nucleotide sequence of human TANGO 222, the nucleotide at position 236 is a cytosine (C). In this embodiment, the amino acid at position 68 is aspartate (D). In another embodiment of a nucleotide sequence of human TANGO 222, the nucleotide at position 305 is thymine (T). In this embodiment, the amino acid at position 91 is aspartate (D). In another embodiment of a nucleotide sequence of human TANGO 222, the nucleotide at position 305 is cytosine (C). In this embodiment, the amino acid at position 91 is glutamate (E). In another embodiment of a nucleotide sequence of human TANGO 222, the nucleotide at position 362 is cytosine (C). In this embodiment, the amino acid at position 110 is aspartate (D). In another embodiment of a nucleotide sequence of human TANGO 222, the nucleotide at position 362 is guanine (G). In this embodiment, the amino acid at position 110 is glutamate (E).

The signal peptide prediction program SIGNALP (Nielsen et al. (1997) Protein Engineering 10:1-6) predicted that TANGO 222 includes a 19 amino acid signal peptide (amino acid 1 to about amino acid 19 of SEQ ED NO:38) preceding the mature TANGO 222 protein (corresponding to about amino acid 20 to amino acid 134 of SEQ ID NO:38). TANGO 222 is predicted to have a molecular weight of 15.1 kDa prior to cleavage of its signal peptide and a molecular weight of 13.1 kDa subsequent to cleavage of its signal peptide.

In certain embodiments, a TANGO 222 family member has the amino acid sequence of SEQ ID NO:38, and the signal sequence is located at amino acids 1 to 17, 1 to 18, 1 to 19, 1 to 20, or 1 to 21. In such embodiments of the invention, the domains and the mature protein resulting from cleavage of such signal peptides are also included herein. For example, the cleavage of a signal sequence consisting of amino acids 1-19, results in a mature TANGO 222 protein corresponding to amino acids 20 to 134. The signal sequence is normally cleaved during processing of the mature protein.

An N-glycosylation site having the sequence NVTM is found from amino acids 27 to of SEQ ID NO:8. A cGMP-dependent protein kinase phosphorylation site having the sequence KKRS is found from amino acids 121 to 124. A protein kinase C phosphorylation site having the sequence SCK is found from amino acids 33 to 35. A second protein kinase C phosphqrylation site having the sequence TLR is found from amino acids 56 to 58. A microbdies C-terminal targeting signal having the sequence SRL is found from amino acids 132 to 134.

A clone, EpT222, which encodes human TANGO 222, was deposited with the American Type Culture Collection (10801 University Boulevard, Manassas, Va. 20110-2209) on Jan. 7, 1999 and assigned Accession Number 207043. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

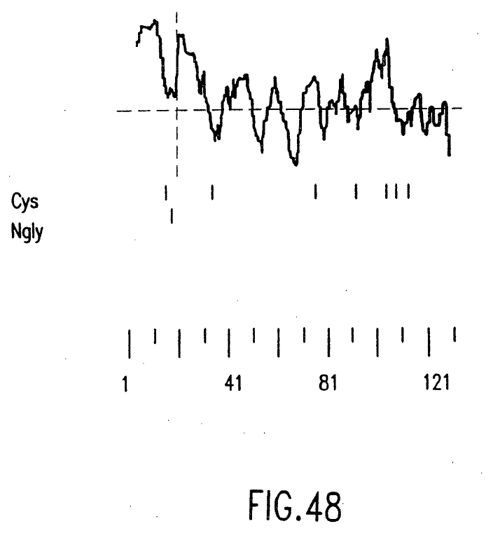

FIG. 48 depicts a hydropathy plot of human TANGO 222. The dashed vertical line separates the signal sequence (amino acids 1-19) on the left from the mature protein (amino acids 20-134) on the right.

### Uses of TANGO 222 Nucleic Acids, Polypeptides, and Modulators Thereof

Because TANGO 222 is expressed in subcutaneous adipose tissue, TANGO 222 polypeptides, nucleic acids, and modulators of TANGO 222 expression or activity can be used to modulate adipocyte function, e.g., fat metabolism. For example, TANGO 222 polypeptides, nucleic acids, and modulators thereof, can be used to modulate the function, morphology, proliferation and/or differentiation of cells in the tissues in which it is expressed. For example, TANGO 222 nucleic acids, proteins and modulators thereof can be utilized to modulate adipocyte function and adipocyte-related processes and disorders such as, e.g., obesity, regulation of body temperature, lipid metabolism, carbohydrate metabolism, body weight regulation, obesity, anorexia nervosa, diabetes mellitus, unusual susceptibility or insensitivity to heat or cold, arteriosclerosis, atherosclerosis, and disorders involving abnormal vascularization, e.g., vascularization of solid tumors. Additionally, such molecules can be used to treat disorders associated with abnormal fat metabolism, e.g., cachexia. In another example, such molecules can be used to treat disorders associated with abnormal proliferation of these tissues, e.g., cancer, e.g., breast cancer or liver cancer. Such molecules can be used to treat disorders associated with abnormal fat metabolism, e.g., obesity, arteriosclerosis, or cachexia.

### Human TANGO 176

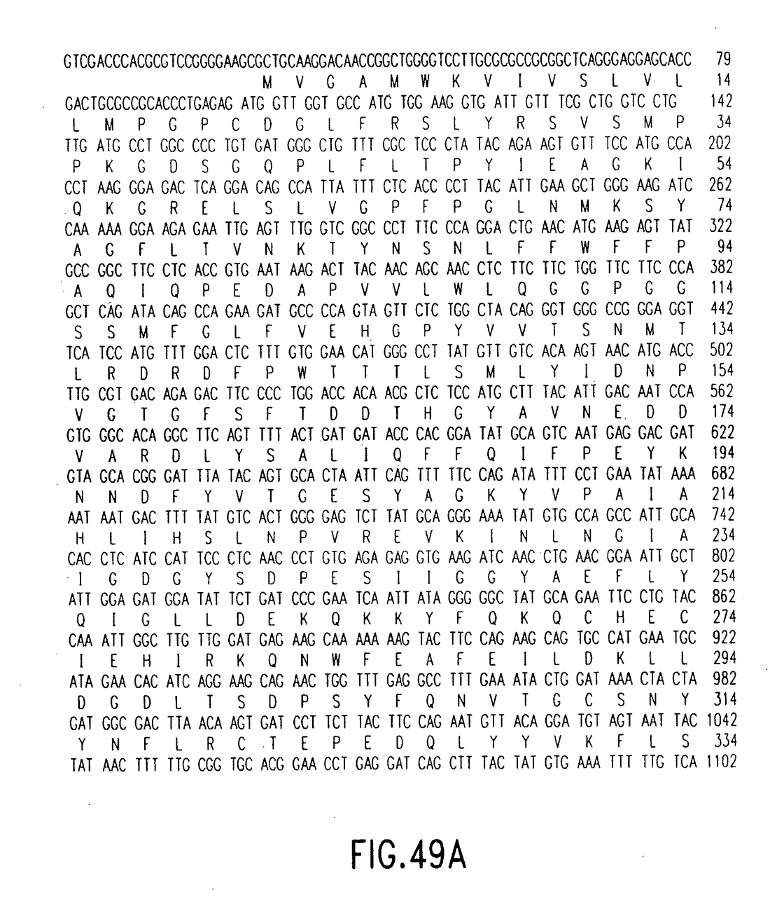

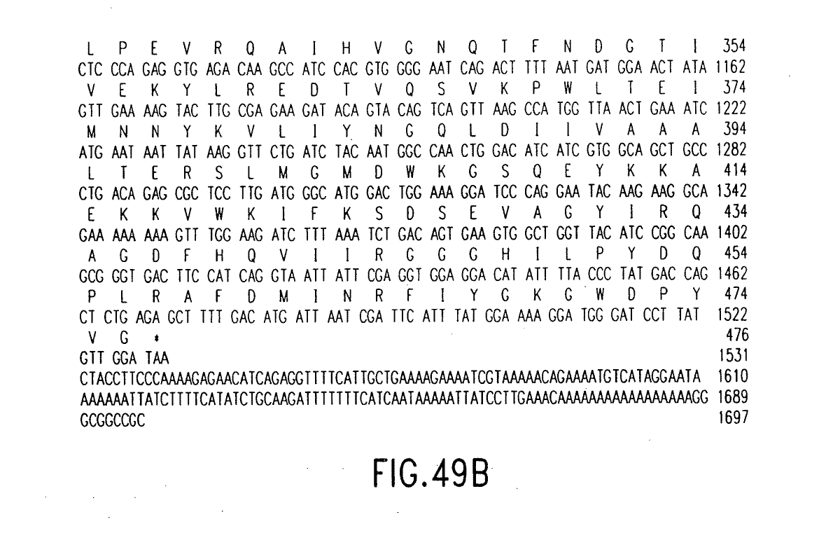

A cDNA encoding human TANGO 176 was identified by analyzing the sequences of clones present in a human pituitary cDNA library. This analysis led to the identification of a clone, Athbb28g6, encoding full-length human TANGO 176. The cDNA of this clone is 1697 nucleotides long (FIG. 49A-49B; SEQ ID NO:39). It is noted that the nucleotide sequence contains Sal I and Not I adapter sequences on the 5′ and 3′ ends, respectively. The open reading frame of this cDNA, nucleotides 101 to 1528, encodes a 476 amino acid secreted protein (SEQ ID NO:40).

In one embodiment of a nucleotide sequence of human TANGO 176, the nucleotide at position 250 is an adenine (A). In this embodiment, the amino acid at position 50 is glutamate (E). In another embodiment of a nucleotide sequence of human TANGO 176, the nucleotide at position 250 is a cytosine (C). In this embodiment, the amino acid at position 50 is aspartate (D). In another embodiment of a nucleotide sequence of human TANGO 176, the nucleotide at position 277 is adenine (A). In this embodiment, the amino acid at position 59 is glutamate (E). In another embodiment of a nucleotide sequence of human TANGO 176, the nucleotide at position 277 is cytosine (C). In this embodiment, the amino acid at position 59 is aspartate (D). In another embodiment of a nucleotide sequence of human TANGO 176, the nucleotide at position 400 is adenine (A). In this embodiment, the amino acid at position 100 is glutamate (E). In another embodiment of a nucleotide sequence of human TANGO 176, the nucleotide at position 400 is cytosine (C). In this embodiment, the amino acid at position 100 is aspartate (D). The signal peptide prediction program SIGNALP (Nielsen et al. (1997) Protein Engineering 10:1-6) predicted that human TANGO 176 includes a 22 amino acid signal peptide (amino acid 1 to about amino acid 22 of SEQ ID NO:40) preceding the mature TANGO 176 protein (corresponding to about amino acid 23 to amino acid 476 of SEQ ID NO:40). Human TANGO 176 is predicted to have a molecular weight of approximately 71 kDa prior to cleavage of its signal peptide and a molecular weight of approximately 68 kDa subsequent to cleavage of its signal peptide.

In certain embodiments, a TANGO 176 family member has the amino acid sequence of SEQ ID NO:40, and the signal sequence is located at amino acids 1 to 19, 1 to 20, 1 to 21, 1 to 22, 1 to 23, or 1 to 24. In such embodiments of the invention, the domains and the mature protein resulting from cleavage of such signal peptides are also included herein. For example, the cleavage of a signal sequence consisting of amino acids 1 to 22, results in a mature TANGO 176 protein corresponding to amino acids 23 to 476. The signal sequence is normally cleaved during processing of the mature protein.

An N-glycosylation site having the sequence NKTY is found from amino acids 81 to 84. A second N-glycosylation site having the sequence NMTL is found from amino acids 132 to 135. A third N-glycosylation site having the sequence NVTG is found from amino acids 307 to 310. A fourth N-glycosylation site having the sequence NQTF is found from amino acids 346 to 349. A protein kinase C phosphorylation site having the sequence TLR is found from amino acids 134 to 136. A second protein kinase C phosphorylation site having the sequence SVK is found from amino acids 366 to 368. A third protein kinase C phosphorylation site having the sequence TER is found from amino acids 396 to 398. A casein kinase II phosphorylation site having the sequence TLRD is found from amino acids 134 to 137. A second casein kinase II phosphorylation site having the sequence SFTD is found from amino acids 160 to 163. A third casein kinase II phosphorylation site having the sequence SDPE is found from amino acids 240 to 243. A fourth casein kinase II phosphorylation site having the sequence TEPE is found from amino acids 321 to 324. A fifth casein kinase II phosphorylation site having the sequence SLPE is found from amino acids 334 to 337. A sixth casein kinase II phosphorylation site having the sequence TFND is found from amino acids 348 to 351. A seventh casein kinase II phosphorylation site having the sequence TIVE is found from amino acids. 353 to 356. An eighth casein kinase II phosphorylation site having the sequence SDSE is found from amino acids 424 to 427. A tyrosine kinase phosphorylation site having the sequence KSDSEVAGY is found from amino acids 423 to 431. An N-myristoylation site having the sequence GLFRSL is found from amino acids 22 to 27. A second N-myristoylation site having the sequence GGPGGS is found from amino acids 110 to 115. A third N-myristoylation site having the sequence GTGFSF is found from amino acids 156 to 161. A fourth N-myristoylation site having the sequence GIAIGD is found from amino acids 232 to 237. A serine active site, e.g., from a serine carboxypeptidase, having the sequence VTGESYAG is found from amino acids 200 to 207. A beta and gamma ‘Greek key’ motif signature, e.g., from crystallins, having the sequence MNNYKVLIYNGQLDII is found from amino acids 375 to 390.

There are four conserved cysteines in the extracellular domain at positions 271, 274, 311, and 320. Human TANGO 176 has a high proportion of charged amino acids in the predicted extracellular (20%, not including histidines) and cytoplasmic (29%) domains. Human TANGO 176 is predicted to have a molecular weight of 54.2 kDa prior to cleavage of its signal peptide and a molecular weight of 51.9 kDa subsequent to cleavage of its signal peptide.

Secretion assays indicate that the polypeptide encoded by human TANGO 176 is secreted. The secretion assays were performed essentially as follows: 8×10⁵ 293T cells were plated per well in a 6-well plate and the cells were incubated in growth medium (DMEM, 10% fetal bovine serum, penicillin/strepomycin) at 37° C., 5% CO₂ overnight. 293T cells were transfected with 2 μg of full-length TANGO 176 inserted in the pMET7 vector/well and 10 μg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16424-54). 1 ml DMEM without methionine and cysteine with 50 μCi Trans-³⁵S (ICN Cat. # 51006) was added to each well and the cells were incubated at 37° C., 5% CO₂ for the appropriate time period. A 150 μl aliquot of conditioned medium was obtained and 150 μl of 2×SDS sample buffer was added to the aliquot. The sample was heat-inactivated and loaded on a 4-20% SDS-PAGE gel. The gel was fixed and the presence of secreted protein was detected by autoradiography.

A clone, EpT176, which encodes human TANGO 176, was deposited as with the American Type Culture Collection (10801 University Boulevard, Manassas, Va. 20110-2209) on Jan. 7, 1999 and assigned Accession Number 207042. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

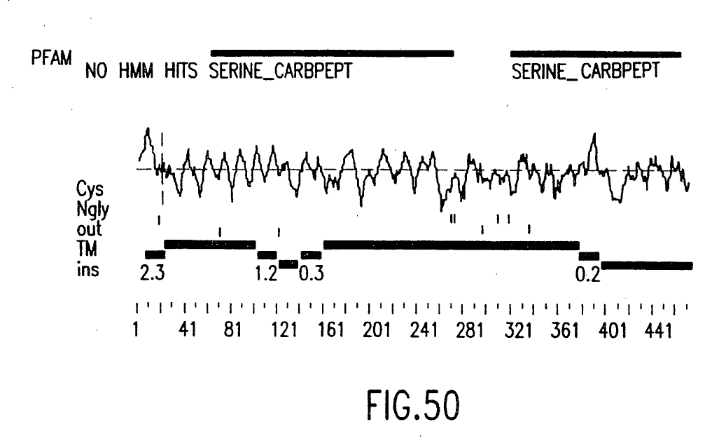

FIG. 50 depicts a hydropathy plot of human TANGO 176. The dashed vertical line separates the signal sequence (amino acids 1-22) on the left from the mature protein (amino acids 23-476) on the right.

A human TANGO 176 polypeptide differs from known molecules (e.g., the serine carboxypeptidase of WO 98/44128) at the sequence KAE found from amino acids 413 to 415. In human TANGO 176, the sequence is KAE. In known molecules, the sequence is AEK. Human TANGO 176 exhibited the most homology with mosquito vitellogenic carboxypetidase.

Northern analysis of human TANGO 176 mRNA revealed expression in a wide range of tissues including heart, spleen, kidney, placenta, and peripheral blood leukocytes. Human TANGO 176 mRNA expression was not detected in the brain, skeletal muscle, colon, thymus, liver, small intestine, and lung.

### Mouse TANGO 176

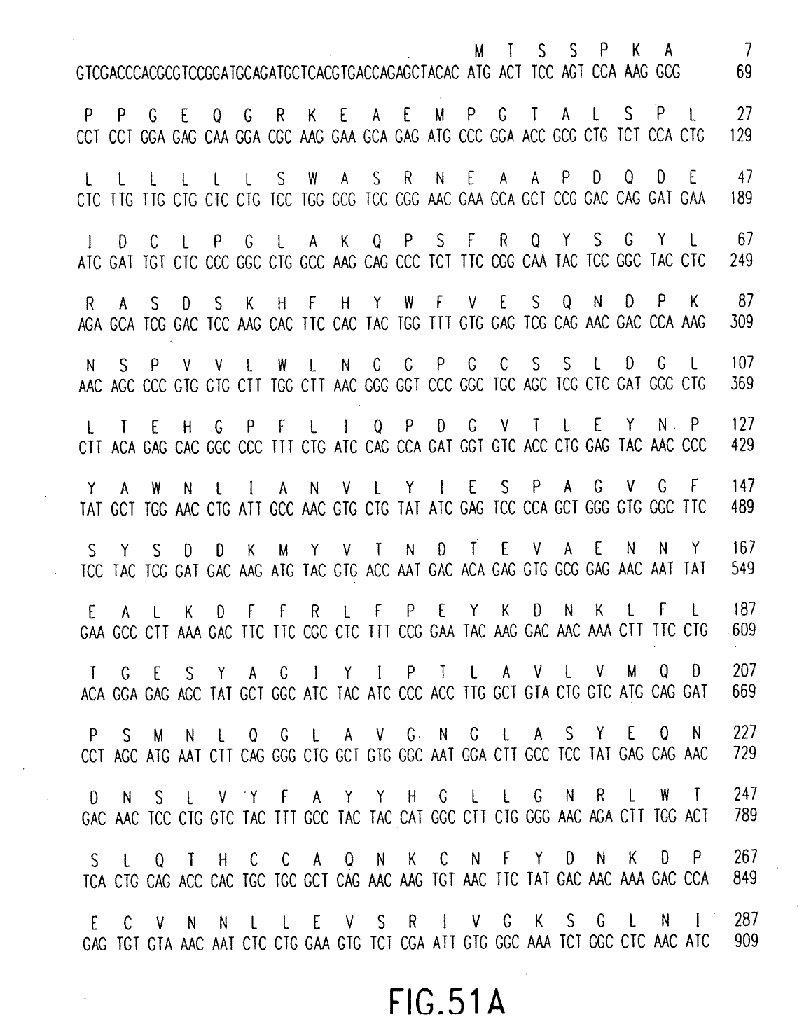

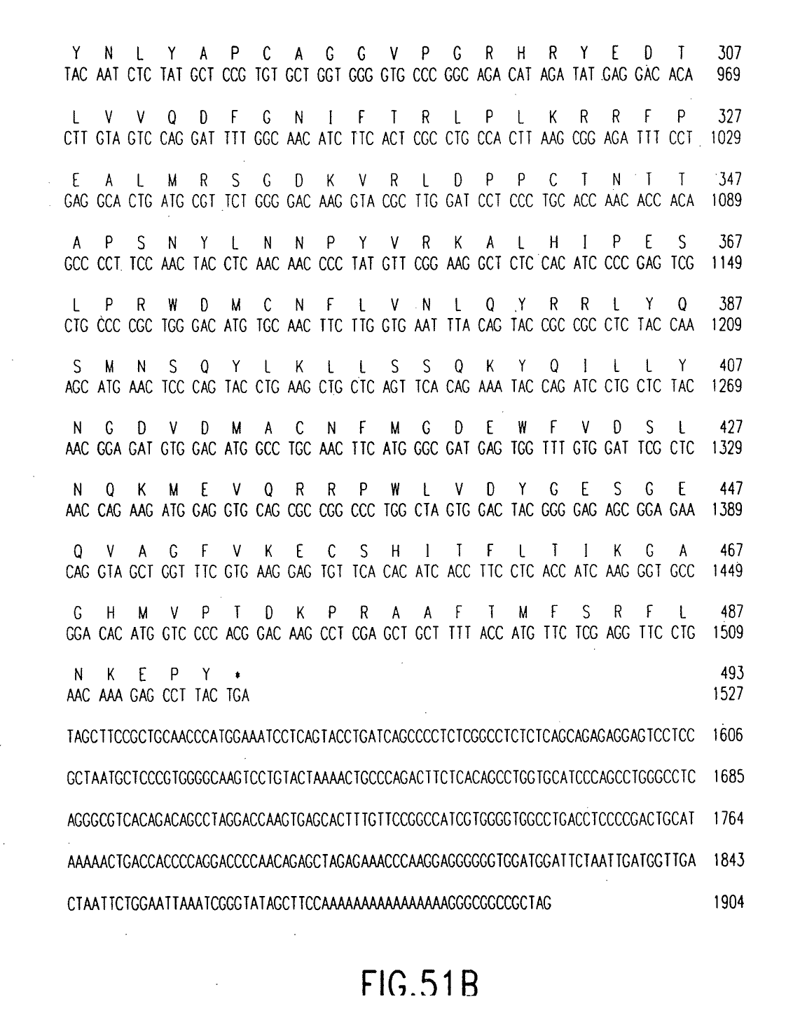

A cDNA encoding mouse TANGO 176 was identified by analyzing the sequences of clones present in a mouse alveolar macrophage cell line cDNA library. This analysis led to the identification of a clone, jtmca099e05 encoding full-length mouse TANGO 176. The mouse TANGO 176 cDNA of this clone is 1904 nucleotides long (FIG. 51A-51B; SEQ ID NO:41). It is noted that the nucleotide sequence contains Sal I and Not I adapter sequences on the 5′ and 3′ ends, respectively. The open reading frame of this cDNA, nucleotides 49 to 1524, encodes a 492 amino acid secreted protein depicted in SEQ ID NO:42.

In one embodiment of a nucleotide sequence of mouse TANGO 176, the nucleotide at position 81 is an guanine (G). In this embodiment, the amino acid at position 11 is glutamate (E). In another embodiment of a nucleotide sequence of mouse TANGO 176, the nucleotide at position 81 is a cytosine (C). In this embodiment, the amino acid at position 11 is aspartate (D). In another embodiment of a nucleotide sequence of mouse TANGO 176, the nucleotide at position 96 is adenine (A). In this embodiment, the amino acid at position 16 is glutamate (E). In another embodiment of a nucleotide sequence of mouse TANGO 176, the nucleotide at position 96 is cytosine (C). In this embodiment, the amino acid at position 16 is aspartate (D). In another embodiment of a nucleotide sequence of mouse TANGO 176, the nucleotide at position 102 is guanine (G). In this embodiment, the amino acid at position 18 is glutamate (E). In another embodiment of a nucleotide sequence of mouse TANGO 176, the nucleotide at position 102 is cytosine (C). In this embodiment, the amino acid at position 18 is aspartate (D).

The signal peptide prediction program SIGNALP (Nielsen et al. (1997) Protein Engineering 10:1-6) predicted that mouse TANGO 176 includes a 41 amino acid signal peptide (amino acid 1 to about amino acid 41 of SEQ ID NO:42) preceding the mature mouse TANGO 176 protein (corresponding to about amino acid 42 to amino acid 492 of SEQ ID NO:42). Mouse TANGO 176 is predicted to have a molecular weight of approximately 74 IcDa prior to cleavage of its signal peptide and a molecular weight of approximately 68 kDa subsequent to cleavage of its signal peptide.

In situ tissue screening was performed on mouse adult and embryonic tissue to analyze for the expression of mouse TANGO 176 mRNA. Expression was observed at moderate to high levels in a number of adult tissues. Expression was generally ubiquitous in positive tissues. Expression during embryogenesis was ubiquitous as well and consistently higher in the liver. A sense control probe was used and had minimal or no signal. Ubiquitous signals were detected in the liver, kidney, adrenal gland, and lymph nodes. A moderate, ubiquitous signal was detected in the submandibular gland. A moderate signal in the mucosal epithelium of the stomach. A signal was observed in the mucosal epithelium and the villi of the small intestine, cortex of the thymus, mucosal epithelium of the colon. A strong signal was observed in the follicles of the spleen. A moderate, ubiquitous signal was observed in the bladder. A moderate signal outlining the seminiferous tubules of the testes was observed. A strong signal was observed in the ovaries. A strong, ubiquitous signal was observed in the placenta. No expression was observed in the following tissues: brain, eye and harderian gland, white fat, brown fat, heart, pancreas, and skeletal muscle.

In the case of embryonic expression, the following results were obtained: At E13.5, E14.5, E15.5, E16.5, E18.5 and P1.5, a signal was observed ubiquitously. The signal was moderate to strong and slightly stronger in the liver.

Human and mouse TANGO 176 sequences exhibit considerable similarity at the protein, nucleic acid, and open reading frame levels. An alignment (made using the ALIGN software {Myers and Miller (1989) CABIOS, ver. 2.0}; BLOSUM 62 scoring matrix; gap penalties −12/−4), reveals a protein identity of 29.8%. The human and mouse TANGO 176 cDNAs are 52.9% identical, as assessed using the same software and parameters as indicated (without the BLOSUM 62 scoring matrix). In the respective ORFs, calculated in the same fashion as the full length cDNAs, human and mouse TANGO 176 are 52.9% identical.

### Use of TANGO 176 Nucleic Acids, Polypeptides and Modulators Thereof

The TANGO 176 protein molecules of the invention comprise a family of proteins with homology to lysosomal protective protein cathepsin A (PPCA), an important enzyme with serine carboxypeptidase activity at lysosomal pH and deamidase/esterase activity at neutral pH. PPCA is thought to be involved in the activation and stabilization of lysosomal β-galactosidase and neuraminidase and can be active extracellularly. PPCA is also thought to affect vaso and neuroactive peptide activity when released, for example, from cells (e.g., blood cells, such as platelets or white blood cells, macrophages, endothelial cells and fibroblasts), in response to stimulation. PPCA may also have chemotactic activity on neutrophils or monocytes when part of a protein complex formed from PPCA, an alternatively spliced P-galactosidase and neuraminidase. Based on the sequence similarity between TANGO 176 proteins and PPCA, TANGO 176 (and members of the TANGO 176 family) likely function in a manner similar to that of PPCA. Thus, TANGO 176 nucleic acids, polypeptides, and modulators thereof, can be used to treat PPCA-associated disorders. For example, PPCA deficiency is associated with lysosomal accumulation of sialyloligosaccharides, e.g., galactosialidosis (Goldberg Syndrome). PPCA deficiency may also be associated with a defect in neutrophil or monocyte chemotaxis. Thus, TANGO 176 polypeptides, nucleic acids, and modulators thereof can be used to treat lysosomal disorders, e.g., sialyloligosaccharide accumulation (e.g., PPCA deficiency or galactosialidosis) and disorders associated with impaired neutrophil or monocyte chemotaxis (e.g., recurrent or chronic bacterial infections).

TANGO 176 is expressed in pituitary tissue. The pituitary secretes such hormones as thyroid stimulating hormone (TSH), follicle stimulating hormone (FSH), adrenocotropic hormone (ACTH), and others. It controls the activity of many other endocrine glands (thyroid, ovaries, adrenal, etc.). Pituitary related disorders include, among others, acromegaly, Cushing's syndrome, craniopharyngiomas, Empty Sella syndrome, hypogonadism, hypopituitarism, and hypophysitis, in addition to disorders of the endocrine glands the pituitary controls.

In another example, TANGO 176 polypeptides, nucleic acids, or modulators thereof, can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

In another example, TANGO 176 polypeptides, nucleic acids, or modulators thereof, can be used to treat disorders of the adrenal medulla, such as neoplasms (e.g., pheochromocytomas, neuroblastomas, and ganglioneuromas).

In another example, TANGO 176 polypeptides, nucleic acids, or modulators thereof, can be used to treat disorders of the thyroid gland, such as hyperthyroidism (e.g., diffuse toxic hyperplasia, toxic multinodular goiter, toxic adenoma, and acute or subacute thyroiditis), hypothyroidism (e.g., cretinism and myxedema), thyroiditis (e.g., Hashimoto's thyroiditis, subacute granulomatous thyroiditis, subacute lymphocytic thyroiditis, Riedel's thyroiditis), Graves' disease, goiter (e.g., simple diffuse goiter and multinodular goiter), or tumors (e.g., adenoma, papillary carcinoma, follicular carcinoma, medullary carcinoma, undifferentiated malignant carcinoma, Hodgkin's disease, and non-Hodgkin's lymphoma).

In another example, TANGO 176 polypeptides, nucleic acids, and modulators thereof can also be used to modulate pituitary function, and thus, to treat disorders associated with abnormal pituitary function. Examples of such disorders include pituitary dwarfism, hyperthyroidism associated with inappropriate thyrotropin secretion, acromegaly, and pituitary growth hormone secreting tumors.

Because TANGO 176 is expressed in the follicles of the spleen, liver, kidney, adrenal gland, lymph node, submandibular gland, mucosal epithelium of the stomach, mucosal epithelium and the villi of the small intestine, cortex of the thymus, and mucosal epithelium of the colon, the TANGO 176 polypeptides, nucleic acids and/or modulators thereof can be used to modulate the function, morphology, proliferation and/or differentiation of cells in the tissues in which it is expressed.

Because TANGO 176 is expressed in the kidney, the TANGO 176 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such molecules can be used to treat or modulate renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As TANGO 176 exhibits expression in the spleen, TANGO 176 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 176 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 176 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

In another example, because TANGO 176 exhibits expression in the liver, TANGO 176 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

As TANGO 176 exhibits expression in the small intestine, TANGO 176 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinernia), obstructive lesions, hernias, intestinal adhesions, intussusception, or volvulus.

### Mouse TANGO 201

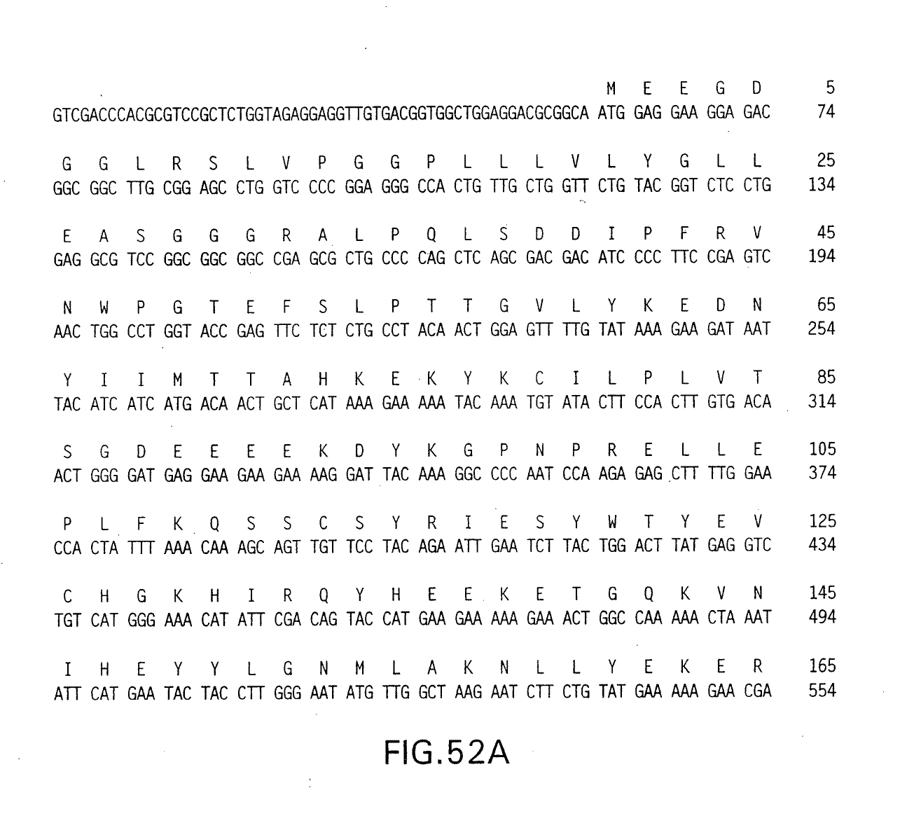

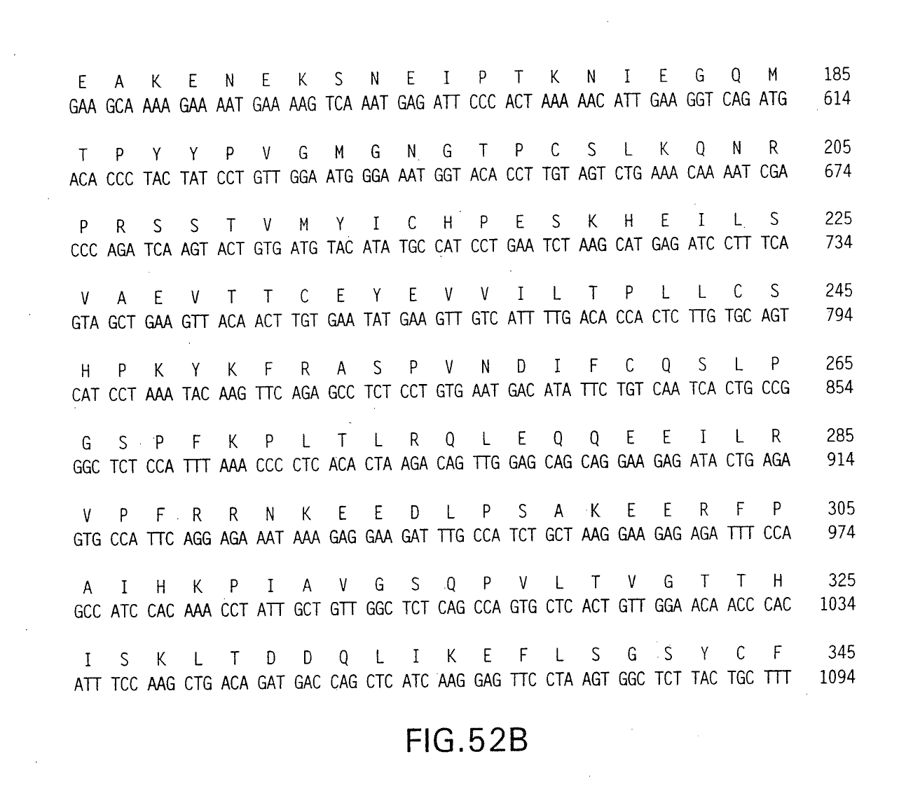

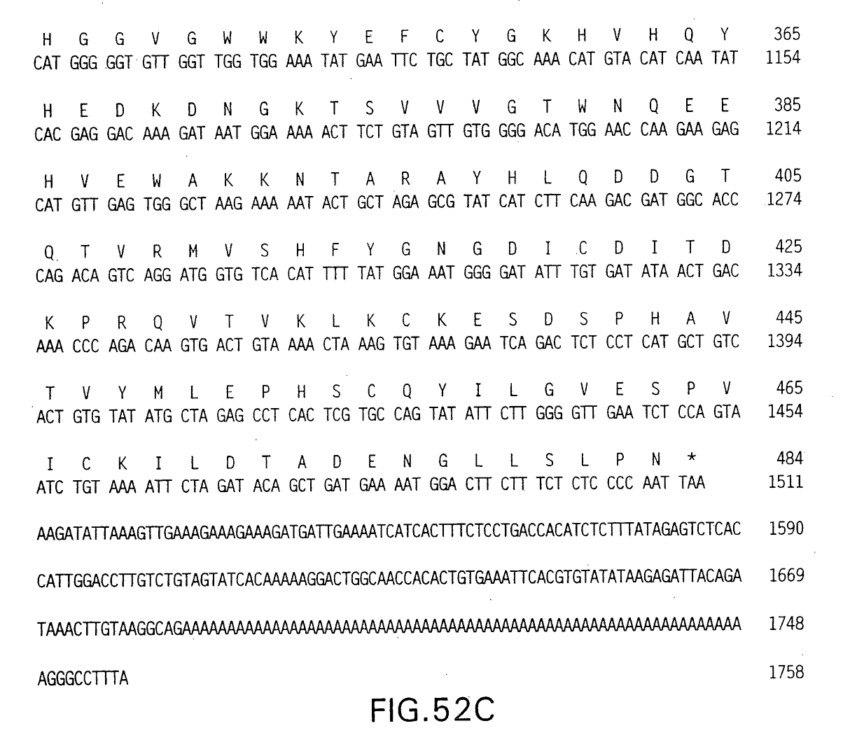

A cDNA clone, AtmMa41h08, encoding mouse TANGO 201 was identified by analysis of EST sequences from a bone marrow stromal cell cDNA library. The cDNA of this clone is 1758 nucleotides long (FIG. 52A-52C; SEQ ID NO:43). The open reading frame of this cDNA (nucleotides 60 to 1508 of SEQ ID NO:43) encodes a 483 amino acid secreted protein (SEQ ID NO:44). It is noted that the nucleotide sequence contains a SalI adapter sequence on the 5′ end.

The signal peptide prediction program SIGNALP (Nielsen et al. (1997) Protein Engineering 10:1-6) predicted that mouse TANGO 201 includes a 33 amino acid signal peptide (amino acid 1 to about amino acid 33 of SEQ ID NO:44) preceding the mature mouse TANGO 201 protein (corresponding to about amino acid 34 to amino acid 483). Mouse TANGO 201 is predicted to have a molecular weight of 54.9 kDa prior to cleavage of its signal peptide and a molecular weight of 51.7 kDa subsequent to cleavage of its signal peptide. The presence of a methionine residue at positions 69, 154, 185, 193, 212, and 449 indicate that there can be alternative forms of mouse TANGO 201 of 415 amino acids, 330 amino acids, 299 amino acids, 291 amino acids, 272 amino acids, and 35 amino acids of SEQ ID NO:44, respectively.

In one embodiment, a mouse TANGO 201 protein (SEQ ID NO:44) contains a signal sequence of about amino acids 1-33 of SEQ ID NO:44.

In certain embodiments, a TANGO 201 family member has the amino acid sequence, and the signal sequence is located at amino acids 1 to 31, 1 to 32, 1 to 33, 1 to 34 or 1 to 35. In such embodiments of the invention, the domains and the mature protein resulting from cleavage of such signal peptides are also included herein. For example, the cleavage of a signal sequence consisting of amino acids 1 to 33 results in a mature TANGO 201 protein corresponding to amino acids 34 to 483 of SEQ ID NO:44. The signal sequence is normally cleaved during processing of the mature protein.

The present invention contemplates mutations, which are either naturally occurring or targeted mutations, in the nucleotide sequence resulting in changes in the polypeptide amino acid sequence. More particularly, mutations can be conservative substitutions of an amino acid or amino acids wherein the resulting polypeptide retains essentially the same functional activity. For example, in one embodiment the TANGO 201 nucleotide at position 65 is a cytosine (C). In this embodiment, the amino acid at position 2 is aspartate (D). In another embodiment of a nucleotide sequence of mouse TANGO 201, the nucleotide at position 74 is a guanine (G). In this embodiment, the amino acid at position 5 is glutamate (E) In another embodiment of a nucleotide sequence of mouse TANGO 201, the nucleotide at position 81 is a guanine (G). In this embodiment, the amino acid at position 8 is a valine (V). In another embodiment of a nucleotide sequence of mouse TANGO 201, the nucleotide at position 93 is an adenine (A). In this embodiment, the amino acid at position 12 is a isoleucine (I).

In another embodiment of a nucleotide sequence of mouse TANGO 201, the nucleotide at position 124 is a thymidine (T). In this embodiment, the amino acid at position 22 is a phenylalanine (F). In another embodiment of a nucleotide sequence of mouse TANGO 201, the nucleotide at position 172 is a cytosine (C). In this embodiment, the amino acid at position 38 is a threonine (T). In another embodiment of a nucleotide sequence of mouse TANGO 201, the nucleotide at position 244 is a guanine (G). In this embodiment, the amino acid at position 62 is an arginine (R). In another embodiment of a nucleotide sequence of TANGO 201, the nucleotide at position 1092 is a thymidine (T). In this embodiment, the amino acid at position 345 is phenylalanine (F). In another embodiment of a nucleotide sequence of mouse TANGO 201, the nucleotide at position 1092 is a cytosine (C). In this embodiment, the amino acid at position 345 is leucine (L) In another embodiment of a nucleotide sequence of mouse TANGO 201, the nucleotide at position 1092 is adenine (A). In this embodiment, the amino acid at position 345 is a isoleucine (I). In another embodiment of a nucleotide sequence of mouse TANGO 201, the nucleotide at position 1092 is guanine (G). In this embodiment, the amino acid at position 345 is a valine (V).

A glycosaminoglycan attachment site having the sequence SGGG is found from amino acids 28 to 31. A cAMP- and cGMP-dependent protein kinase C(PKC) phosphorylation site having the sequence KKNT is found from amino acids 391 to 394.

A PKC phosphorylation site having the sequence SYR is found from amino acids 114 to 16. A second PKC phosphorylation site having the sequence SLK is found from amino acids 200 to 202. A third PKC phosphorylation site having the sequence TLR is found from amino acids 273 to 275. A fourth PKC phosphorylation site having the sequence SAK is found from amino acids 298 to 300. A fifth PKC phosphorylation site having the sequence TAR is found from amino acids 394 to 396. A sixth PKC phosphorylation site having the sequence TVR is found from amino acids 407 to 409. A seventh PKC phosphorylation site having the sequence TDK is found from amino acids 424 to 426. An eighth PKC phosphorylation site having the sequence TVK is found from amino acids 431 to 433.

A casein kinase II (CKII) phosphorylation site having the sequence TSGD is found from amino acids 85 to 88. A second CKII phosphorylation site having the sequence SKHE is found from amino acids 219 to 222. A third CKII phosphorylation site having the sequence SVAE is found from amino acids 225 to 228. A fourth CKII phosphorylation site having the sequence TTCE is found from amino acids 230 to 233. A fifth CKH phosphorylation site having the sequence SAKE is found from amino acids 298 to 301. A sixth CKII phosphorylation site having the sequence TADE is found from amino acids 472 to 475.

An N-myristoylation (N-MRTL) site having the sequence GGLRSL is found from amino acids 6 to 11. A second N-MRTL site having the sequence GLLEAS is found from amino acids 23 to 28. A third N-MRTL site having the sequence GGGRAL is found from amino acids 29 to 34. A fourth N-MRTL site having the sequence GTEFSL is found from amino acids 49 to 54. A fifth N-MRTL site having the sequence GQKVNI is found from amino acids 141 to 146. A sixth N-MRTL site having the sequence GNMLAK is found from amino acids 152 to 157. A seventh N-MRTL site having the sequence GMGNGT is found from amino acids 192 to 197.

FIG. 53 depicts a hydropathy plot of mouse TANGO 201. The dashed vertical line separates the signal sequence (amino acids 1-33) on the left from the mature protein (amino acids 34-483) on the right.

### Human TANGO 201

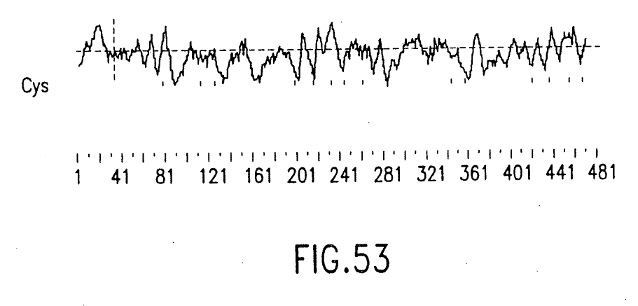

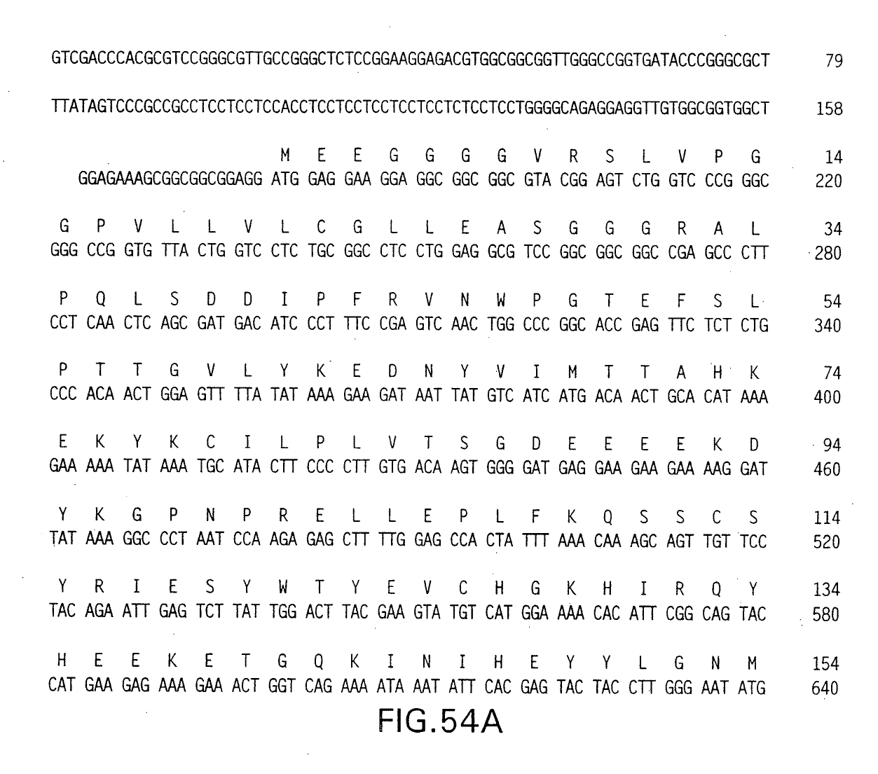

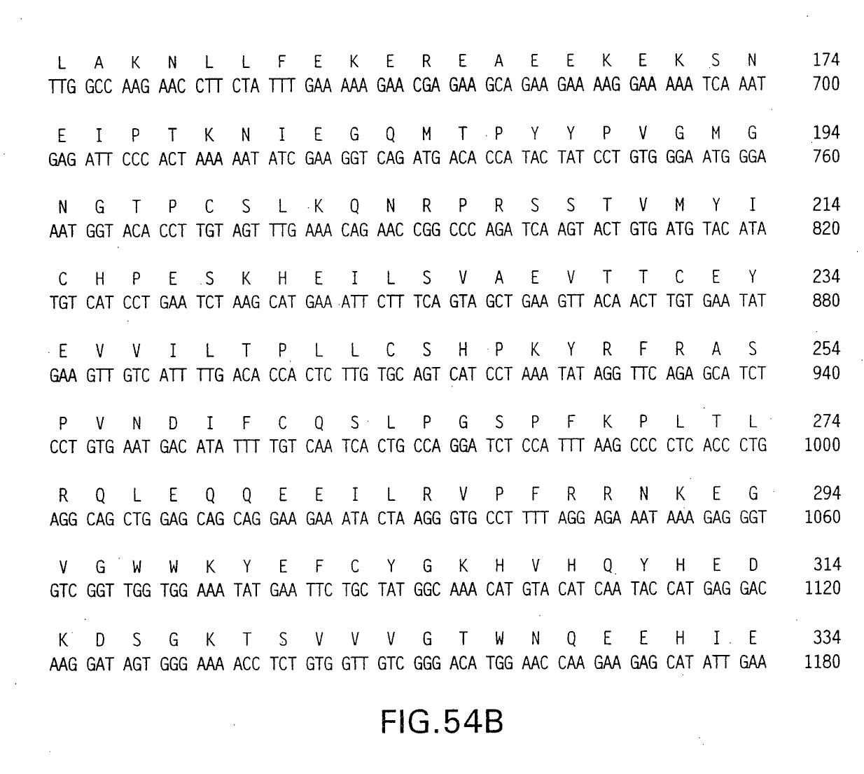

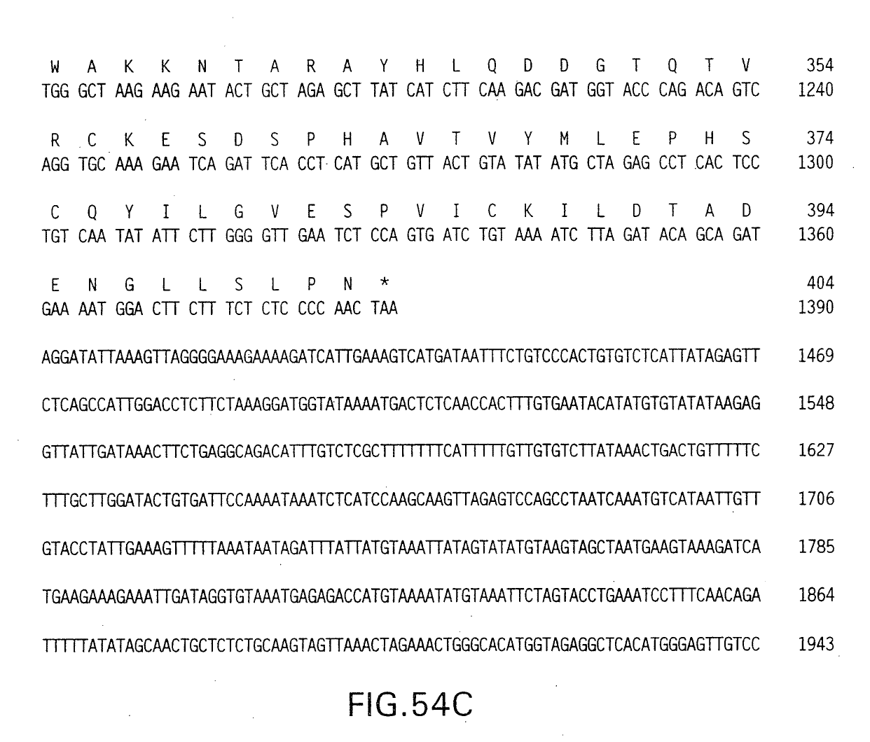

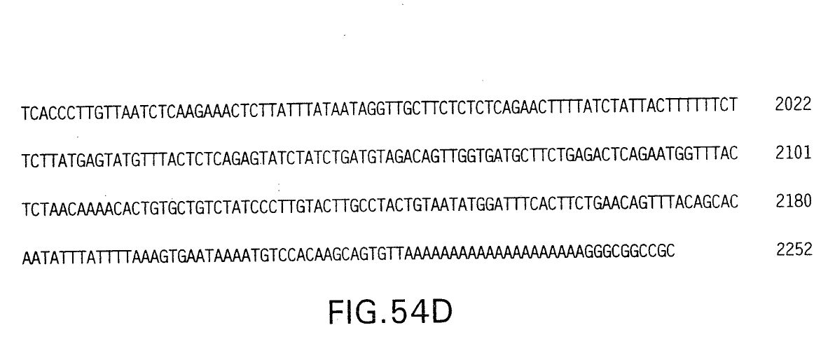

A cDNA clone, Athbb012c06, encoding human TANGO 201 was identified using mouse TANGO 201 cDNA probes on a pituitary library. The human TANGO 201 clone is 2252 nucleotides long (FIG. 54A-54D; SEQ ID NO:45). The open reading frame of the cDNA (nucleotides 179 to 1387 of SEQ ID NO:45) encodes a 403 amino acid protein shown in SEQ ID NO:46. It is noted that the human TANGO 201 nucleotide sequence contains Sal I and Not I adapter sequences on the 5′ and 3′ ends, respectively.

The signal peptide prediction program SIGNALP (Nielsen, et al. (1997) Protein Engineering 10:1-6) predicted that human TANGO 201 includes a 33 amino acid signal peptide (amino acid 1 to about amino acid 33 of SEQ ID NO:46) preceding the mature human TANGO 201 protein (corresponding to about amino acid 34 to amino acid 403 of SEQ ID NO:46). Human TANGO 201 is predicted to have a molecular weight of 45.9 kDa prior to cleavage of its signal peptide and a molecular weight of 42.8 kDa subsequent to cleavage of its signal peptide. The presence of a methionine residue at positions 69, 154, 185, 193, and 212 indicate that there can be alternative forms of human TANGO 201 of 335 amino acids, 250 amino acids, 219 amino acids, 211 amino acids, and 192 amino acids of SEQ ID NO:46, respectively.

In one embodiment, a human TANGO 201 protein (SEQ ID NO:46) contains a signal sequence of about amino acids 1-33 of SEQ ID NO:46.

In certain embodiments, a TANGO 201 family member has the amino acid sequence, and the signal sequence is located at amino acids 1 to 31, 1 to 32, 1 to 33, 1 to 34 or 1 to 35. In such embodiments of the invention, the domains and the mature protein resulting from cleavage of such signal peptides are also included herein. For example, the cleavage of a signal sequence consisting of amino acids 1 to 33 results in a mature TANGO 201 protein corresponding to amino acids 34 to 403 of SEQ ID NO:46. The signal sequence is normally cleaved during processing of the mature protein.

A glycosaminoglycan attachment site having the sequence SGGG is found from amino acids 28 to 31. A cAMP- and cGMP-dependent protein kinase phosphorylation site having the sequence KKNT is found from amino acids 337 to 340. A protein kinase C (PKC) phosphorylation site having the sequence SYR is found from amino acids 114 to 116. A second PKC phosphorylation site having the sequence SLK is found from amino acids 200 to 202. A third PKC phosphorylation site having the sequence TLR is found from amino acids 273 to 275. A fourth PKC phosphorylation site having the sequence SGK is found from amino acids 317 to 319. A fifth PKC phosphorylation site having the sequence TAR is found from amino acids 340 to 342. A sixth PKC phosphorylation site having the sequence TVR is found from amino acids 353 to 355. A casein kinase II (CKII) phosphorylation site having the sequence TSGD is found from amino acids 85 to 88. A second CKII phosphorylation site having the sequence SKHE is found from amino acids 219 to 222. A third CKII phosphorylation site having the sequence SVAE is found from amino acids 225 to 228. A fourth CKII phosphorylation site having the sequence TTCE is found from amino acids 230 to 233. A fifth CKII phosphorylation site having the sequence TADE is found from amino acids 392 to 395. An N-myristoylation (N-MRTL) site having the sequence GGVRSL is found from amino acids 6 to 11. A second N-MRTL site having the sequence GLLEAS is found from amino acids 23 to 28. A third N-MRTL site having the sequence GGGRAL is found from amino acids 29 to 34. A fourth N-MRTL site having the sequence GTEFSL is found from amino acids 49 to 54. A fifth N-MRTL site having the sequence GQKINI is found from amino acids 141 to 146. A sixth N-MRTL site having the sequence GNMLAK is found from amino acids 152 to 157. A seventh N-MRTL site having the sequence GMGNGT is found from amino acids 192 to 197.

Clone Athbb012c06, which encodes human TANGO 201, was deposited as a composite deposit with the American Type Culture Collection (10801 University Boulevard, Manassas, Va. 20110-2209) on Jan. 22, 1999 and assigned Accession Number 207081. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

FIG. 55 depicts a hydropathy plot of human TANGO 201. The dashed vertical line separates the signal sequence (amino acids 1-33) on the left from the mature protein (amino acids 34-403) on the right.

Tissue Distribution of TANGO 201 mRNA

Tissue distribution of TANGO 201 mRNA was determined by Northern blot hybridization performed under standard conditions and washed under stringent conditions, i.e., 0.2×SSS at 65° C. RNA from various human tissues were as provided in Multiple Tissue Northern Blots (MTN Blots, Clontech Laboratories, Inc., Palo Alto Calif.). The results indicated that human TANGO 201 mRNA is expressed in multiple human tissues, including pancreas, testis, adrenal medulla, adrenal cortex, kidney, liver, thyroid, brain, skeletal muscle, placenta, heart, lung, and stomach. The detection of TANGO 201 mRNA in a wide range of human normal tissues suggests that TANGO 201 has an essential cellular function. Two transcripts were observed of approximately 2.0 and 2.5 kb, consistent with the suggestion of alternative splicing raised by the sequence alignment. Furthermore, the ratios of these two forms differs among the tissues. For example, the 2.0 kb transcript predominates in adrenal medulla whereas the 2.5 kb form predominates in thyroid. This suggests tissue specific expression of spliced forms of human TANGO 201.

In situ tissue screening was performed on mouse adult and embryonic tissue to analyze the expression of mouse TANGO 201 mRNA. Expression in the adult mouse was not detected in any tissues tested.

### Similarities Between Mouse and HUMAN TANGO 201 and to Other Sequences

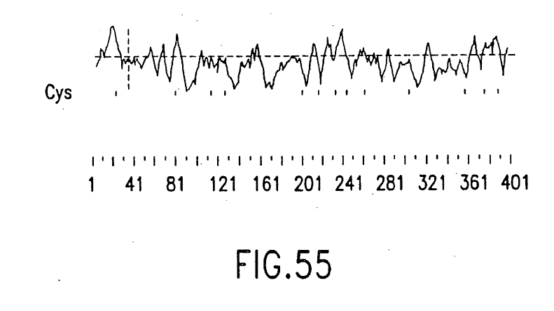

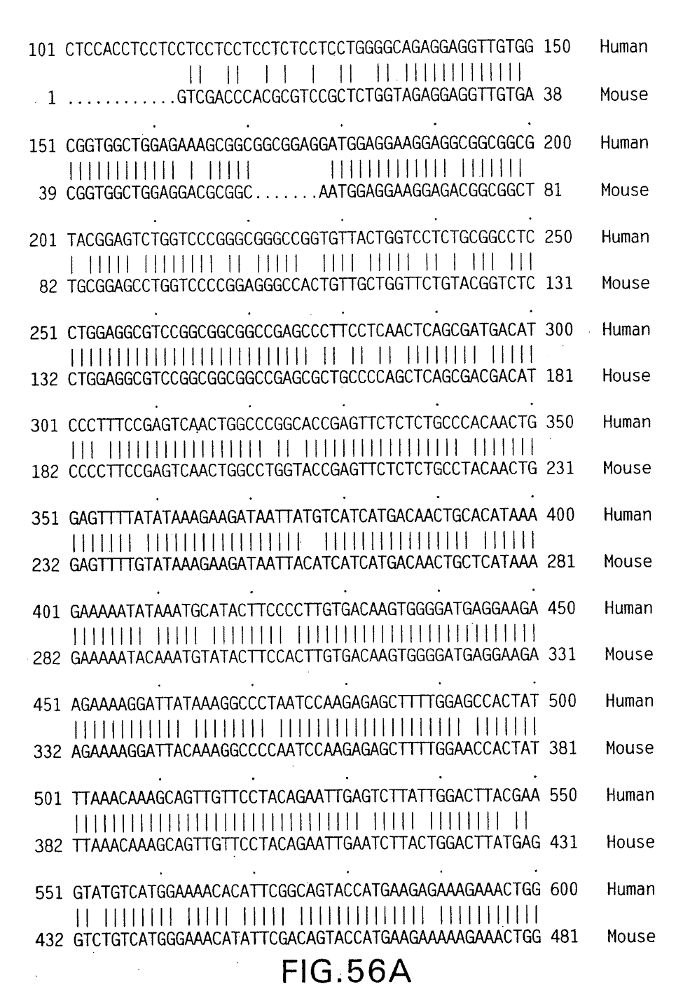

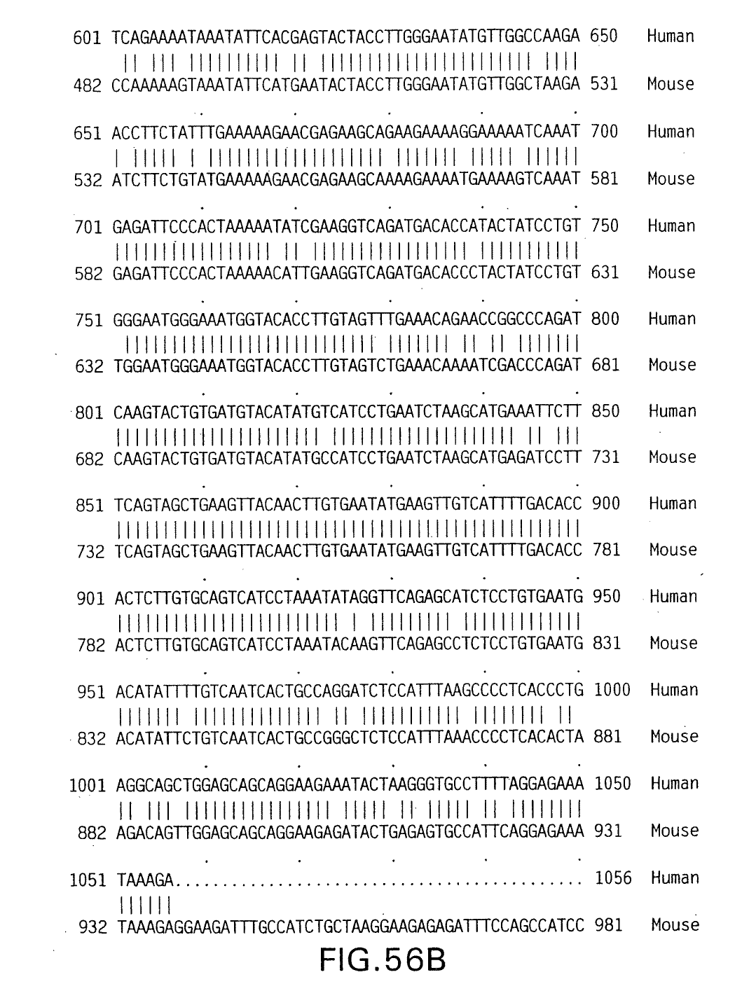

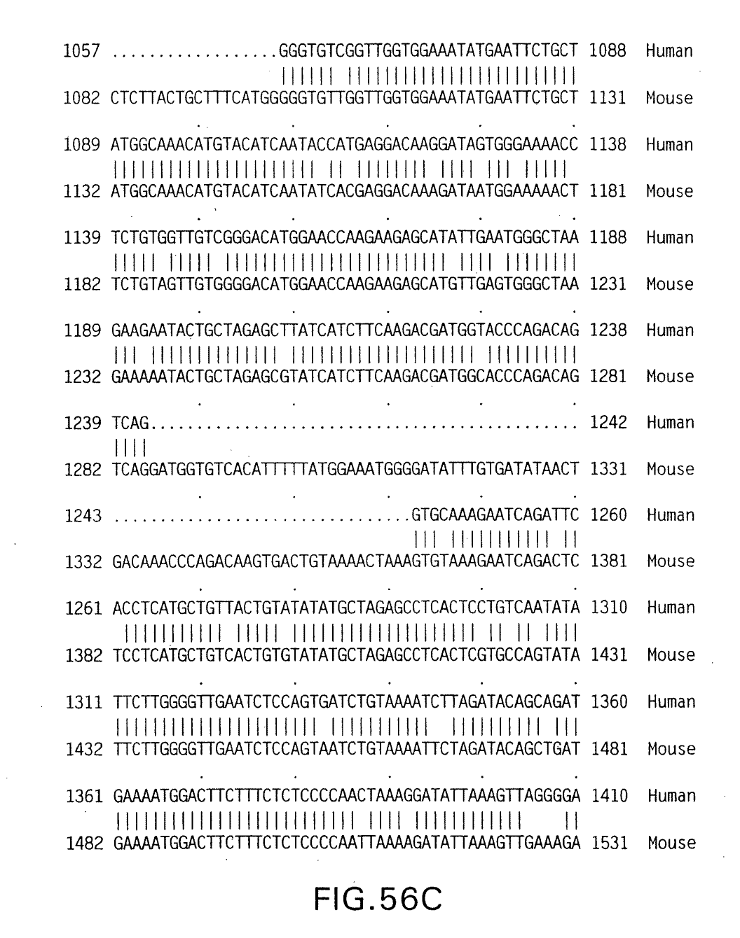

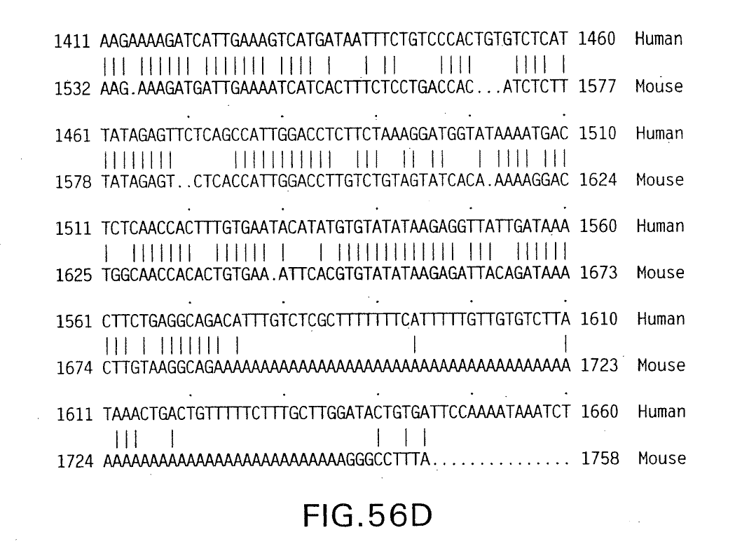

An alignment of the nucleotide sequences of mouse TANGO 201 (nucleotides 1-1758 of SEQ ID NO:43) and human TANGO 201 (nucleotides 101-1660 of SEQ ID NO:45) using the program GAP (Needleman and Wunsch (1970) J. Mol. Biol. 48:443-453) in GCG (Wisconsin Package Version 9.1, Genetics Computer Group, Madison Wis.) with a score matrix of nwsgapdna, a gap penalty 50, and a gap extension penalty 3 resulted in an identity of 84.8%. The mouse sequence differs from the human sequence by the presence of two inserted sequences. The first is a 162 base insertion from nucleotide 938 to 1100 and the second is 78 bases from nucleotide 1286 to 1363 of SEQ ID NO:43. This alignment is shown in FIG. 56A-56D.

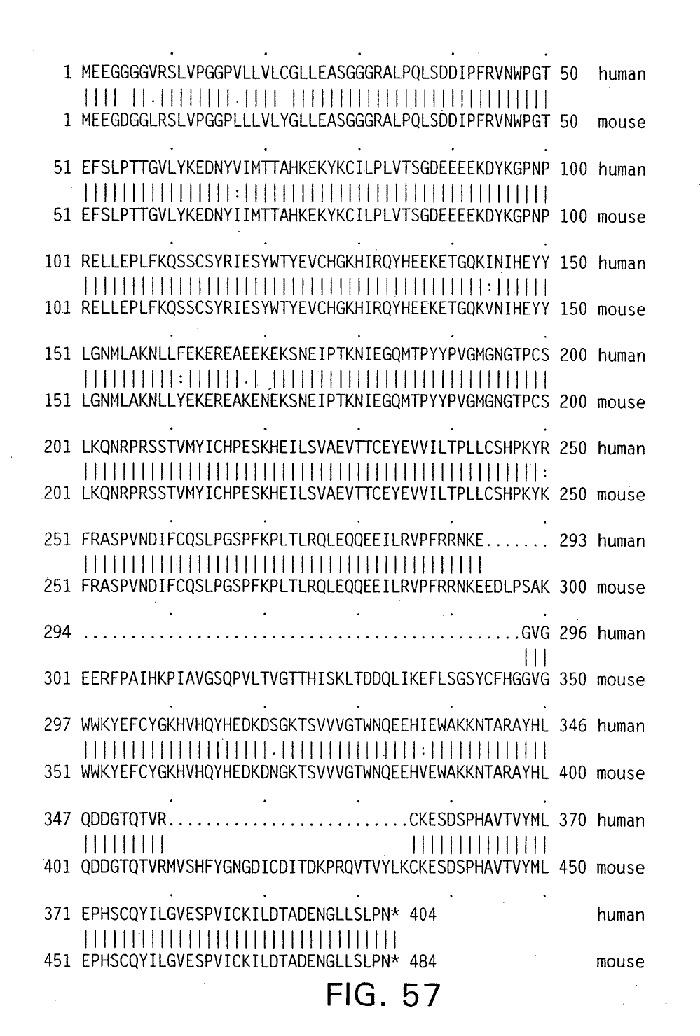

The amino acid sequences of mouse TANGO 201 (amino acids 1483; SEQ ID NO:44) and human TANGO 201 (amino acids 1-403; SEQ ID NO:46) were aligned and analyzed using the program GAP (Needleman and Wunsch (1970) J. Mol. Biol. 48:443-453) in GCG (Wisconsin Package Version 9.1, Genetics Computer Group, Madison Wis.). An identity of 97% was seen in which the program settings were a score matrix of blosum 62, a gap penalty 12, and a gap extension penalty 4. The mouse sequence differs from the human sequence by the presence of two inserted sequences. The first is a 54 residue insertion from amino acid 294 to 347 and the second is 26 residues from amino acid 410 to 435. This alignment is shown in FIG. 57.

In one embodiment, the invention contemplates alternative splicing of the mRNA encoding a TANGO 201 protein. For example, one embodiment of the invention includes a human TANGO 201 nucleotide sequence further comprising exons which encode for a polypeptide sequence which is similar to the mouse TANGO 201 polypeptide sequence between amino acids 294 to 247 and 410 to 435, in the same relative position of the polypeptide of SEQ ID NO:44. Further, the invention also features splicing of the mouse TANGO 201, that is, mouse TANGO 201 is alternatively spliced so that the mRNA encoding the polypeptide has deletions corresponding to amino acids 294 to 247 and 410 to 435 of SEQ ID NO:44.

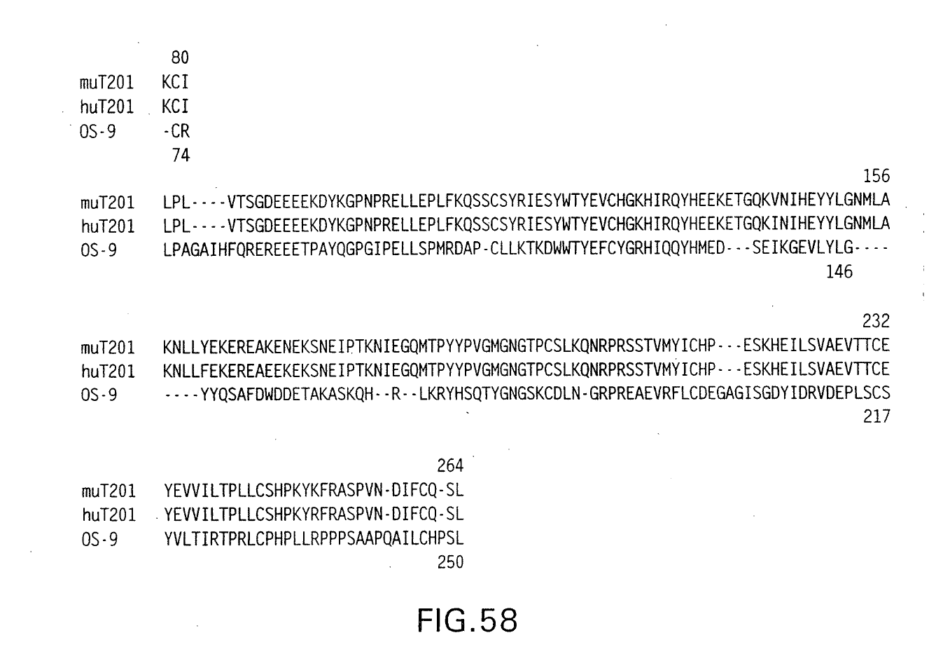

Mouse and human TANGO 201 show homology to OS-9, a putative secreted human protein believed to be involved in cell growth (Su, et al., (1966) Mol. Carcinogenesis. 15:270-275; Kimura, et al., (1998) J. Biochem. 123:876-882). FIG. 58 depicts an alignment of a portion of mouse TANGO 201 amino acid sequence (amino acids 78 to 264 of SEQ ID NO:44) and a portion of human TANGO 201 amino acid sequence (amino acids 78 to 264 of SEQ ID NO:46) with a portion of OS-9 (amino acids 73 to 250 of SwissProt Accession No. Q13438). The alignment reveals that the homology is restricted to the N-terminus in which a conserved cysteine-rich domain as defined below is found. The conserved cysteine residues are highlighted in boldface type.

An alignment of human or mouse TANGO 201 with the above-described portion of the OS-9 protein sequence (Q13438) reveals 39.0% identity between human TANGO 201 and OS-9, and 42.2% identity between mouse TANGO 201 and OS-9. This alignment was performed using the ALIGN alignment program with a BLOSUM62 scoring matrix, a gap length penalty of 10, and a gap penalty of 0.05.

As used herein, a cysteine-rich domain of a TANGO 201 polypeptide includes about 140-215 amino acid residues, preferably about 150-205 amino acid residues, more preferably about 155-200 amino acid residues, and most preferably about 165-190 amino acid residues. Typically, a cysteine-rich domain is found at the N-terminal half of TANGO 201 and includes a cluster of about 4-12 cysteine residues conserved in TANGO 201 protein family members, more preferably about 6-10 cysteine residues, and still more preferably about 8 cysteine residues. In addition, a cysteine-rich domain includes at least the following consensus sequence: C-Xaa(n1)-C-Xaa(n2)-C-Xaa(n3)-C-Xaa(n4)-C-Xaa(n4)-C-Xaa(n2)-C-Xaa(n4)-C, wherein C is a cysteine residue, Xaa is any amino acid, n1 is about 20-50 amino acid residues, more preferably about 25-45 amino acid residues, and more preferably 30-40 amino acid residues in length, n2 is about 2-20 amino acid residues, more preferably 5-15 amino acid residues, and more preferably 11-12 amino acid residues in length, n3 is about 40-90 amino acid residues, more preferably about 50-80 amino acid residues, and more preferably 55-75 amino acid residues in length, and n4 is about 5-25 amino acid residues, more preferably 8-20 amino acid residues, and more preferably 13-21 amino acid residues in length.

In one embodiment, a TANGO 201 family member includes a cysteine-rich domain having an amino acid sequence that is at least about 55%, preferably at least about 65%, more preferably at least about 75%, yet more preferably at least about 85%, and most preferably at least about 95% identical to amino acids 79 to 261, to amino acids 79 to 261 or to amino acids 68 to 178, which are the cysteine-rich domains of mouse and human TANGO 201, respectively.

In another embodiment, a TANGO 201 family member includes a cysteine-rich domain having an amino acid sequence that is at least about 55%, preferably at least about 65%, more preferably at least about 75%, yet more preferably at least about 85%, and most preferably at least about 95% identical to amino acids 79 to 261, includes a conserved cluster of 8 cysteine residues, and a cysteine-rich domain consensus sequence as described herein. In yet another embodiment, a TANGO 201 family member includes a cysteine-rich domain having an amino acid sequence that is at least 55%, preferably at least about 65%, more preferably at least about 75%, yet more preferably at least about 85%, and most preferably at least about 95% identical to amino acids 79 to 261 of SEQ ID NO:44 or SEQ ID NO:46, includes a conserved cluster of 8 cysteine residues, a cysteine-rich consensus sequence as described herein, and has at least one TANGO 201 biological activity as described herein.

In a preferred embodiment, a TANGO 201 family member has the amino acid sequence wherein the cluster of conserved cysteine residues is located within amino acid residues 79 to 261 (at positions 79, 113, 126, 199, 215, 232, 244, and 261 of SEQ ID NO:44 or SEQ ID NO:46), and the cysteine-rich domain consensus sequence is located at amino acid residues 79 to 261.

### Uses of TANGO 201 Nucleic Acids, Polypeptides, and Modulators Thereof

TANGO 201 polypeptides, nucleic acids, and modulators thereof, can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which TANGO 201 is expressed include, for example, pancreas, adrenal medulla, adrenal cortex, kidney, thyroid, testis, stomach, heart, brain, liver, placenta, lung, skeletal muscle, or small intestine.

For example, such molecules can be used to treat proliferative disorders, i.e., neoplasms or tumors (e.g., a carcinoma, a sarcoma, adenoma, or myeloid leukemia).

In another example, TANGO 201 polypeptides, nucleic acids, or modulators thereof, can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

In another example, TANGO 201 polypeptides, nucleic acids, or modulators thereof, can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

In another example, TANGO 201 polypeptides, nucleic acids, or modulators thereof, can be used to treat disorders of the adrenal medulla, such as neoplasms (e.g., pheochromocytomas, neuroblastomas, and ganglioneuromas).

In another example, TANGO 201 polypeptides, nucleic acids, or modulators thereof, can be used to treat disorders of the thyroid gland, such as hyperthyroidism (e.g., diffuse toxic hyperplasia, toxic multinodular goiter, toxic adenoma, and acute or subacute thyroiditis), hypothyroidism (e.g., cretinism and myxedema), thyroiditis (e.g., Hashimoto's thyroiditis, subacute granulomatous thyroiditis, subacute lymphocytic thyroiditis, Riedel's thyroiditis), Graves' disease, goiter (e.g., simple diffuse goiter and multinodular goiter), or tumors (e.g., adenoma, papillary carcinoma, follicular carcinoma, medullary carcinoma, undifferentiated malignant carcinoma, Hodgkin's disease, and non-Hodgkin's lymphoma).

In another example, TANGO 201 polypeptides, nucleic acids, or modulators thereof, can be used to treat gastric disorders, such as congenital anamolies (e.g., diaphragmatic hernias, pyloric stenosis, gastric diverticula, and gastric dilatation), gastritis (e.g., acute mucosal inflammation, chronic fundal gastritis, chronic antral gastritis, hypertrophic gastritis, granulomatous gastritis, eosinophilic gastritis), ulcerations (e.g., peptic ulcers, gastric ulcers, and duodenal ulcers), or tumors (e.g., benign polyps, malignant carcinoma, argentaffinomas, carcinoids, gastrointestinal lymphomas, carcomas, and metastatic carcinoma).

In another example, TANGO 201 polypeptides, nucleic acids, or modulators thereof, can be used to treat placental disorders, such as toxemia of pregnancy (e.g., preeclampsia and eclampsia), placentitis, or spontaneous abortion.

In another example, TANGO 201 polypeptides, nucleic acids, or modulators thereof, can be used to treat pulmonary disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchio-alveolar carcinoma, bronchial carcinoid, and mesenchymal tumors).

In another example, TANGO 201 polypeptides, nucleic acids, or modulators thereof, can be used to treat disorders of skeletal muscle, such as muscular dystrophy (e.g., Duchenne muscular dystrophy, Becker Muscular Dystrophy, Emery-Dreifuss muscular dystrophy, Limb-Girdle muscular dystrophy, Facioscapulohumeral muscular dystrophy, myotonic dystrophy, oculopharyngeal muscular dystrophy, distal muscular dystrophy, and congenital muscular dystrophy), motor neuron diseases (e.g., amyotrophic lateral sclerosis, infantile progressive spinal muscular atrophy, intermediate spinal muscular atrophy, spinal bulbar muscular atrophy, and adult spinal muscular atrophy), myopathies (e.g., inflammatory myopathies (e.g., dermatomyositis and polymyositis), myotonia congenita, paramyotonia congenita, central core disease, nemaline myopathy, myotubular myopathy, and periodic paralysis), and metabolic diseases of muscle (e.g., phosphorylase deficiency, acid maltase deficiency, phosphofructokinase deficiency, Debrancher enzyme deficiency, mitochondrial myopathy, carnitine deficiency, carnitine palmityl transferase deficiency, phosphoglycerate kinase deficiency, phosphoglycerate mutase deficiency, lactate dehydrogenase deficiency, and myoadenylate deaminase deficiency).

In another example, TANGO 201 polypeptides, nucleic acids, or modulators thereof, can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

In another example, TANGO 201 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 201 polypeptides, nucleic acids, or modulators thereof, can be used to treat hepatic disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 201 polypeptides, nucleic acids, or modulators thereof, can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflaimmatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 201 polypeptides, nucleic acids, or modulators thereof, can be used to treat testicular disorders, such as unilateral testicular enlargement (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

In another example, TANGO 201 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

### Human TANGO 223

A clone, Athua075b02, encoding full-length human TANGO 223 was identified by use of a partial clone encoding a signal peptide and obtained by use of a yeast signal trap method. This methodology, described, for example, in WO99/24616 dated May 20, 1999, takes advantage of the fact that molecules such as TANGO 223 have an amino terminal signal sequence that directs certain secreted and membrane-bound proteins through the cellular secretory apparatus.

Briefly, a cDNA library from human fetal kidney was prepared in pBOSS1 and transformed into the yeast strain Yscreen2 as described in WO99/24616. cDNA inserts of plasmids rescued from the resulting yeast colonies after selection on glucose were sequenced. The initial signal trap clone obtained, ZmhKy398, was shown to encode a 29 amino acid signal peptide, followed by a 13 amino acid open reading frame. This clone was then fused to a yeast KRE9 gene lacking a functional signal sequence and used to search proprietary databases for a full length clone.

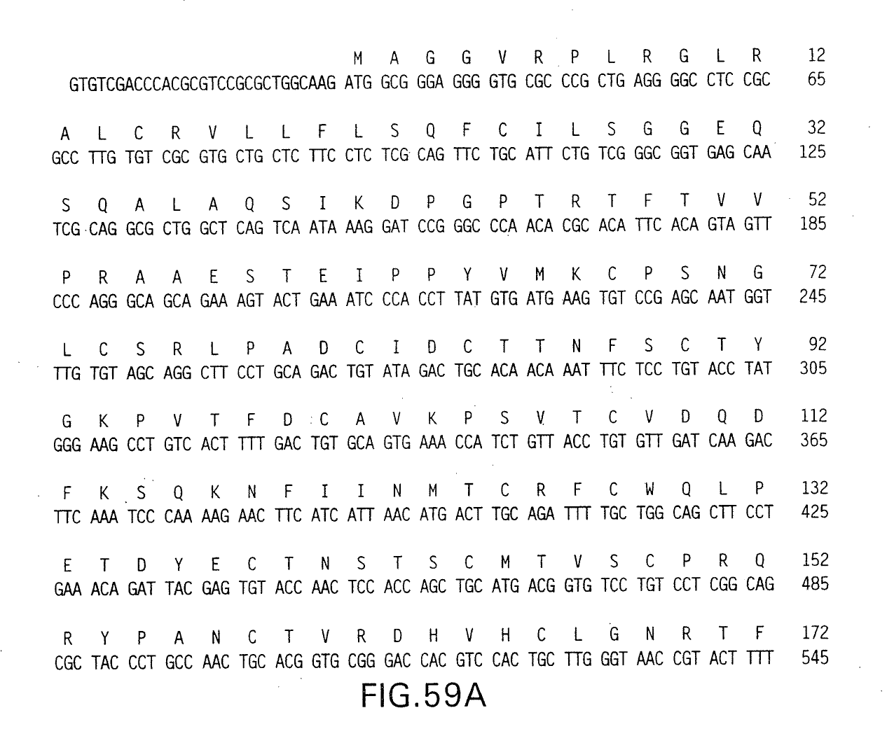

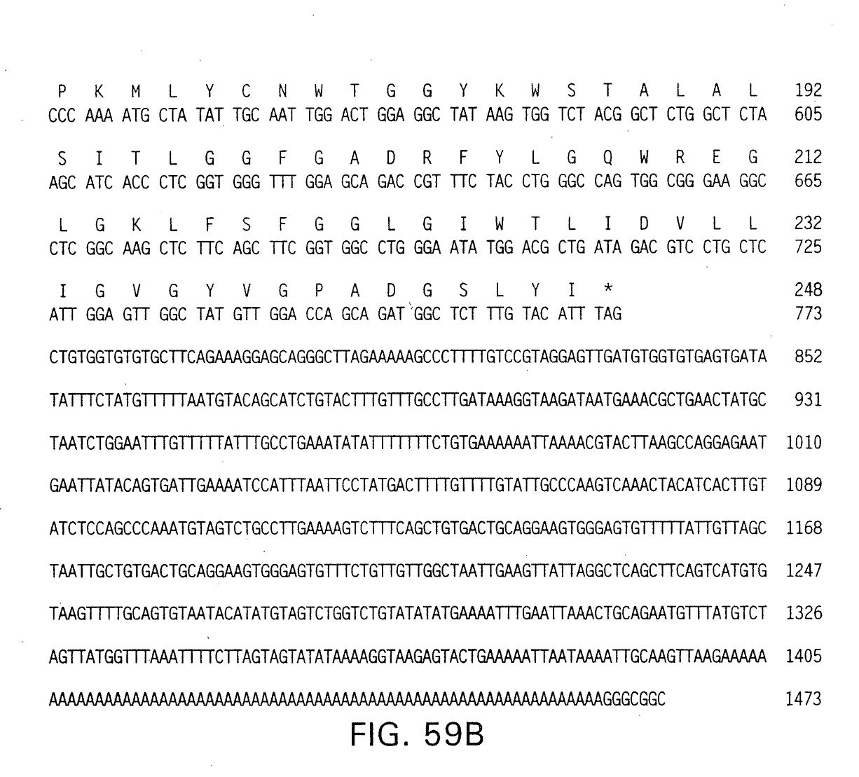

A clone representing an extension of the initial signal sequence positive clone was identified in a human fetal lung library. The cDNA of this clone is 1473 nucleotides long (FIG. 59A-59B; SEQ ID NO:47). The open reading frame of this cDNA, nucleotides 30 to 770, encodes a 247 amino acid protein (SEQ ID NO:48). TANGO 223 is predicted to be a transmembrane protein having a 186 amino acid extracellular domain (amino acids 30-215 of SEQ ID NO:48), a single 23 amino acid transmembrane domain (amino acids 216-238 of SEQ ID NO:48), and a nine amino acid cytoplasmic domain (amino acids 239-247 of SEQ ID NO:48). Alternatively, in another embodiment, the TANGO 223 protein contains an extracellular domain at amino acid residues 239 to 247 of SEQ ID NO:48, a transmembrane domain at amino acid residues 216 to 238, and a cytoplasmic domain at amino acid residues 30 to 215. In addition, there are 15 cysteines in the extracellular domain at positions 68, 74, 81, 84, 90, 100, 108, 125, 128, 138, 144, 149, 158, 166, and 178 and two in the signal peptide sequence at positions 15 and 25 of SEQ ID NO:48.

The signal peptide prediction program SIGNALP (Nielsen et al. (1997) Protein Engineering 10:1-6) predicted that TANGO 223 includes a 29 amino acid signal peptide (amino acid 1 to about amino acid 29 of SEQ ID NO:48) preceding the mature TANGO 223 protein (corresponding to about amino acid 30 to amino acid 247 of SEQ ID NO:48). Human TANGO 223 is predicted to have a molecular weight of 27.2 kDa prior to cleavage of its signal peptide and a molecular weight of 24 kDa subsequent to cleavage of its signal peptide. The presence of a methionine residue at positions 66, 123, 145, and 175 indicate that there can be alternative forms of TANGO 223 of 182 amino acids, 125 amino acids, 103 amino acids, and 73 amino acids, respectively.

In another embodiment, a human TANGO 223 protein (SEQ ID NO:48) contains a signal sequence of about amino acids 1-29 of SEQ ID NO:48. The signal sequence is cleaved during processing of the mature protein.

In another example, a TANGO 223 family member also includes one or more of the following domains: (1) an extracellular domain; (2) a transmembrane domain; and (3) a cytoplasmic domain. Thus, in one embodiment, a TANGO 223 protein contains an extracellular domain of about amino acids 30-215 of SEQ ID NO:48. In another embodiment, a TANGO 223 protein contains a transmembrane domain of about amino acids 216-238 of SEQ ID NO:48. In another embodiment, a TANGO 223 protein contains a cytoplasmic domain of about amino acids 239-247 of SEQ ID NO:48. Alternatively, in another embodiment, a TANGO 223 protein contains an extracellular domain at amino acid residues 239 to 247, a transmembrane domain at amino acid residues 216 to 238, and a cytoplasmic domain at amino acid residues 30 to 215 of SEQ ID NO:48.

In another embodiment, a TANGO 223 protein contains a 169 amino acid extracellular domain (amino acids 30198 of SEQ ID NO:48), a single 23 amino acid transmembrane domain (amino acids 199-221 of SEQ ID NO:48), and a nine amino acid cytoplasmic domain (amino acids 222-230 of SEQ ID NO:48). Alternatively, in another embodiment, the TANGO 223 protein contains an extracellular domain at amino acid residues 222 to 230, a transmembrane domain at amino acid residues 199 to 221, and a cytoplasmic domain at amino acid residues 30 to 198 of SEQ ID NO:48.

In certain embodiments, a TANGO 223 family member has the amino acid sequence, and the signal sequence is located at amino acids 1 to 27, 1 to 28, 1 to 29, 1 to 30 or 1 to 31. In such embodiments of the invention, the domains and the mature protein resulting from cleavage of such signal peptides are also included herein. For example, the cleavage of a signal sequence consisting of amino acids 1 to 29 results in a mature TANGO 223 protein corresponding to amino acids 30 to 247. The signal peptide sequence is normally cleaved during processing of the mature protein.

In one embodiment of a nucleotide sequence of human TANGO 223, the nucleotide at position 98 is guanine (G). In this embodiment, the amino acid at position 57 is a glutamate (E). In another embodiment of a nucleotide sequence of human TANGO 223, the nucleotide at position 98 is a thymidine (T). In this embodiment, the amino acid at position 57 is stop codon resulting in a truncated protein of 57 amino acids length In another embodiment of a nucleotide sequence of human TANGO 223, the nucleotide at position 98 is a cytosine (C). In this embodiment, the amino acid at position 57 is glutamine (Q) In another embodiment of a nucleotide sequence of human TANGO 223, the nucleotide at position 98 is adenine (A). In this embodiment, the amino acid at position 57 is a lysine (K).

An N-glycosylation (N-GCL) site having the sequence NFSC is found from amino acids 87 to 90. A second N-GCL site having the sequence NMTC is found from amino acids 122 to 125. A third N-GCL site having the sequence NSTS is found from amino acids 140 to 143. A fourth N-GCL site having the sequence NCTV is found from amino acids 157 to 160. A fifth N-GCL site having the sequence NRTF is found from amino acids 169 to 172. A sixth N-GCL site having the sequence NWTG is found from amino acids 179 to 182. A protein kinase C(PKC) phosphorylation site having the sequence SIK is found from amino acids 39 to 41. A second PKC phosphorylation site having the sequence SQK is found from amino acids 115 to 117. A third PKC phosphorylation site having the sequence TCR is found from amino acids 124 to 126. A fourth PKC phosphorylation site having the sequence TVR is found from amino acids 159 to 161. A casein kinase II (CKII) phosphorylation site having the sequence SGGE is found from amino acids 28 to 31. A second CKII phosphorylation site having the sequence SIKD is found from amino acids 39 to 42. A third CKII phosphorylation site having the sequence TCVD is found from amino acids 107 to 110. A fourth CKII phosphorylation site having the sequence TYDE is found from amino acids 134 to 137. A fifth CKII phosphorylation site having the sequence TVRD is found from amino acids 159 to 162. A sixth CKII phosphorylation site having the sequence TLID is found from amino acids 226 to 229. An N-myristoylation site having the sequence GGEQSQ is found from amino acids 29 to 34. A second N-myristoylation site having the sequence GGFGAD is found from amino acids 197 to 202.

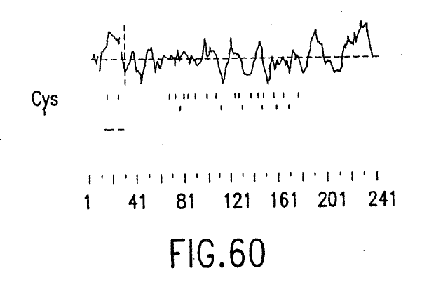

FIG. 60 depicts a hydropathy plot of TANGO 223. The dashed vertical line separates the signal sequence (amino acids 1-29 of SEQ ID NO:48) on the left from the mature protein (amino acids 30-247 of SEQ ID NO:48) on the right.

The human TANGO 223 gene was mapped on radiation hybrid panels to chromosome 15, in the region q26. Flanking markers for this region are WI-3162 and WI-4919. The OTS (otosclerosis) locus also maps to this region of the human chromosome. The ALDH6 (aldehyde dehydrogenase 6), CHRM5 (cholinergic receptor), STX 15(sialyltransferase X), and IDDM3 (insulin-dependent diabetes mellitus 3) genes also map to this region of the human chromosome. This region is syntenic to mouse chromosome 7. The tp (taupe) locus also maps to this region of the mouse chromosome. The agc (shhtrvsn), hf (hepatic fusion), sur (sulfonylurea receptor), and fah (fumarylacetoacetate hyrdrolase) genes also map to this region of the mouse chromosome.

Clone Athua075b02, which encodes TANGO 223, was deposited as a composite deposit with the American Type Culture Collection (10801 University Boulevard, Manassas, Va. 20110-2209) on Jan. 22, 1999 and assigned Accession Number 207081. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Mouse TANGO 223

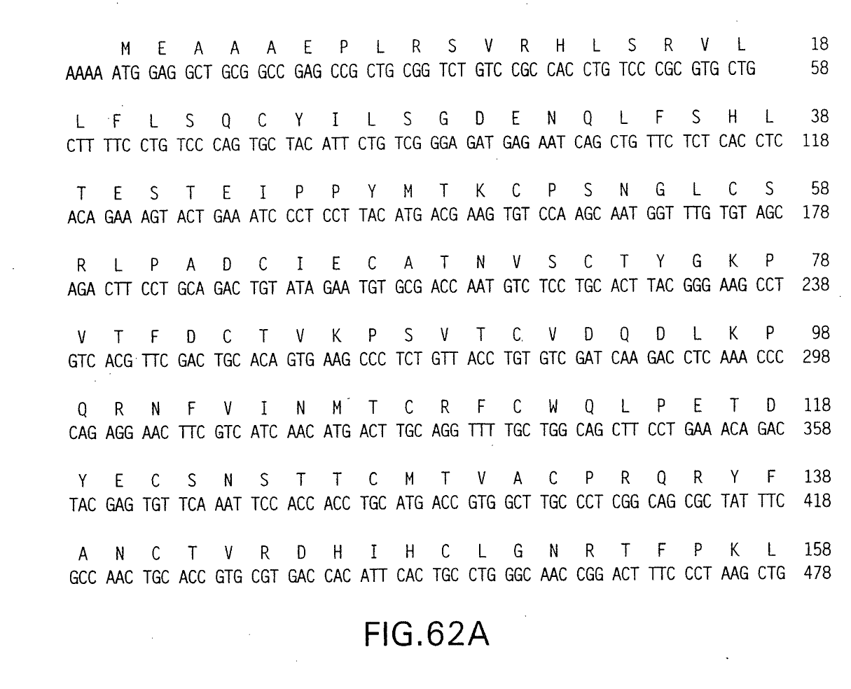

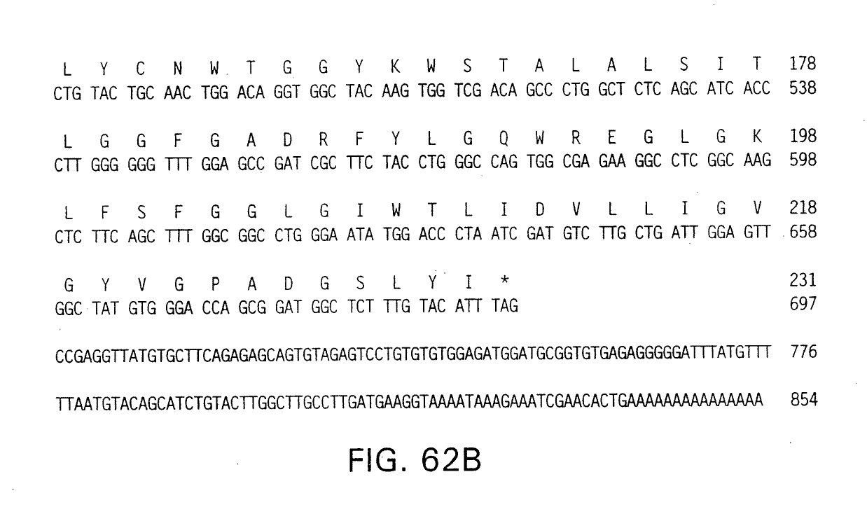

A mouse TANGO 223 clone, Aompa001h06, was identified using the cDNA of the human TANGO 223 as a probe in a screen of a mouse pancreatic library. Mouse TANGO 223 is 854 nucleotides long (FIG. 62A-62B; SEQ ID NO:49). The open reading frame of this cDNA (nucleotides 5 to 694 of SEQ ID NO:49) encodes a 230 amino acid protein (SEQ ID NO:50).

The signal peptide prediction program SIGNALP (Nielsen et al. (1997) Protein Engineering 10:1-6) predicted that TANGO 223 includes a 29 amino acid signal peptide (amino acid 1 to about amino acid 29 of SEQ ID NO:50) preceding the mature TANGO 223 protein (corresponding to about amino acid 30 to amino acid 230 of SEQ ID NO:50). Mouse TANGO 223 is predicted to have a molecular weight of 25.6 kDa prior to cleavage of its signal peptide and a molecular weight of 22.4 kDa subsequent to cleavage of its signal peptide. The presence of a methionine residue at positions 48, 106 and 128 indicate that there can be alternative forms of TANGO 223 of 183 amino acids, 125 amino acids and 103 amino acids of SEQ ID NO:50, respectively.

In certain embodiments, a TANGO 223 family member has the amino acid sequence, and the signal sequence is located at amino acids 1 to 27, 1 to 28, 1 to 29, 1 to 30 or 1 to 31. In such embodiments of the invention, the domains and the mature protein resulting from cleavage of such signal peptides are also included herein. For example, the cleavage of a signal sequence consisting of amino acids 1 to 29 results in a mature TANGO 223 protein corresponding to amino acids 30 to 247. The signal peptide sequence is normally cleaved during processing of the mature protein.

TANGO 223 is predicted to be a transmembrane protein having a 169 amino acid extracellular domain (amino acids 30-198), a single 23 amino acid transmembrane domain (amino acids 199-221 of SEQ ID NO:50), and a nine amino acid cytoplasmic domain (amino acids 222-230 of SEQ ID NO:50). Alternatively, in another embodiment, the TANGO 223 protein contains an extracellular domain at amino acid residues 222 to 230, a transmembrane domain at amino acid residues 199 to 221, and a cytoplasmic domain at amino acid residues 30 to 198 of SEQ ID NO:50. There are 14 cysteines in the extracellular domain at positions 51, 64, 67, 73, 83, 91, 108, 111, 121, 127, 132, 141, 149 and 161 and one in the signal peptide sequence at position 24 of SEQ ID NO:50.

An N-glycosylation (N-GCL) site having the sequence NVSC is found in TANGO 223 from amino acids 70 to 73. A second N-GCL site having the sequence NMTC is found from amino acids 105 to 108. A third N-GCL site having the sequence NSTT is found from amino acids 123 to 126. A fourth N-GCL site having the sequence NCTV is found from amino acids 140 to 143. A fifth N-GCL site having the sequence NRTF is found from amino acids 152 to 155. A sixth N-GCL site having the sequence NWTG is found from amino acids 162 to 165.

A protein kinase C(PKC) phosphorylation site having the sequence SVR is found from amino acids 10 to 12. A second PKC phosphorylation site having the sequence TVK is found from amino acids 84 to 86. A third PKC phosphorylation site having the sequence TCR is found from amino acids 107 to 109. A fourth PKC phosphorylation site having the sequence TVR is found from amino acids 142 to 144.

A casein kinase II (CKII) phosphorylation site having the sequence SGDE is found from amino acids 28 to 31. A second CKII phosphorylation site having the sequence TCVD is found from amino acids 90 to 93. A third CKII phosphorylation site having the sequence TDYE is found from amino acids 117 to 120. A fourth CKII phosphorylation site having the sequence TVRD is found from amino acids 142 to 145. A fifth CKII phosphorylation site having the sequence TLID is found from amino acids 209 to 212.

An N-myristoylation site having the sequence GGFGAD is found from amino acids 180 to 185.

Tissue Distribution of TANGO 223 mRNA

Tissue distribution of TANGO 223 mRNA was determined by Northern blot hybridization performed under standard conditions and washed under stringent conditions, i.e., 0.2×SSS at 65° C. RNA from various human and mouse tissues were as provided in Multiple Tissue Northern Blots (MTN Blots, Clontech Laboratories, Inc., Palo Alto Calif.).

TANGO 223 is expressed in multiple human tissues and hybridizes to nucleic acids in mouse tissues, including heart, brain, liver, kidney, testis, prostate, ovary, small intestine, colon, and peripheral blood leukocytes. TANGO 223 mRNA has highest expression in adult brain and the submandibular gland. Expression was also observed in the testes in a pattern that outlined the seminiferous vesicles. A single transcript of approximately 1 kb was detected in these tissues. The detection of TANGO 223 mRNA in a wide range of normal tissues suggests that TANGO 223 has an essential cellular function. Embryonic mouse tissues also had a ubiquitous signal.

In situ tissue screening was performed on mouse adult and embryonic tissue to analyze the expression of TANGO 223 mRNA.

In the case of adult expression, the following results were obtained: For the testis, a signal outlining some seminiferous tubules was detected. In the placenta, a signal was very weak. In the ovaries, a very weak signal was detected. A weak signal was detected from the adrenal gland. A moderate, ubiquitous signal was detected in the submandibular gland. A moderate signal was detected in the brain. A weak signal was detected in the spinal cord. A weak signal was detected in the lymph node. and a moderate signal was observed in the stomach. No signal was detected in the following tissues: eye and harderian gland, white and brown fat, heart, lung, liver, kidney, colon, small intestine, thymus, spleen, pancreas, skeletal muscle, and bladder.

Embryonic expression was seen in a number of tissues. The highest expressing tissue was the brain and spinal cord which was seen at E13.5 and continues to P1.5. At E15.5, the strongest signal observed was in the brain, spinal cord, lung and kidney. At E16.5, the signal was the same as in E15.5. At E18.5, the signal is highest in the brain, spinal cord, eye and submaxillary gland and kidney. At P1.5, the signal pattern is identical to E18.5.

### Similarity of TANGO 223 to Other Polypeptides

The orientation of the N-terminus toward the extracellular domain indicates TANGO 223 as being a type I transmembrane protein. A BLASTp search (version 1.4.10MP-WashU, Altschul, et al., (1990) J. Mol. Biol. 215:403-410) of the amino acid sequence of TANGO 223 revealed similarity to two Caenorhabditis elegans proteins. One protein, Swiss-Prot accession number 001975 and gene name C41D11.5, is a putative 85.1 kDa nuclease belonging to the family of DNA/RNA nonspecific endonucleases. However, the domain characteristic of this family of proteins is not seen in TANGO 223. Another protein, Swiss-Prot accession number P34280 and gene name C02F5.3, is a putative 64.3 kd GTP-binding protein in chromosome III belonging to the GTP1/OBG family.

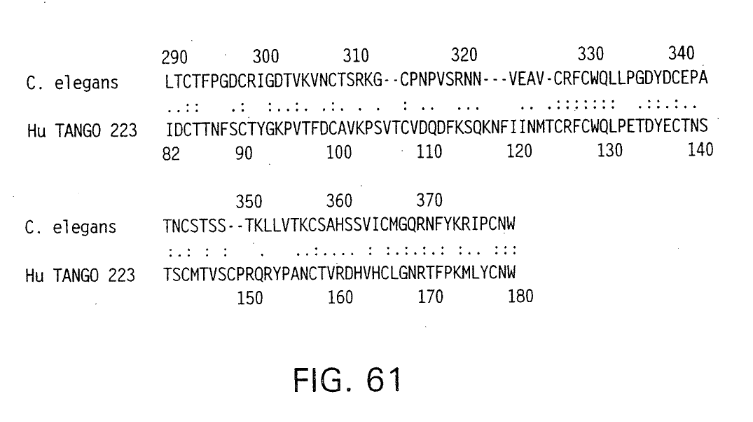

TANGO 223 contains a cysteine-rich domain in which multiple N-glycosylation sites are also present. A homologous cysteine-rich domain is found in the polypeptide sequence of SwissProt 001975. FIG. 61 depicts an alignment of a portion of human TANGO 223 amino acid sequence (amino acids 83 to 178 of SEQ ID NO:48) with amino acids 258 to 376 of SwissProt 001975. The conserved cysteine residues are highlighted in boldface type. A double dot between two residues indicates a complete identity, and a single dot indicates a conservative substitution.

Human TANGO 223 aligned with SwissProt 001975 reveals a sequence identity of 37.5% over a portion polypeptides corresponding to amino acids 82 to 180. This alignment was performed using the ALIGN alignment program with a BLOSUM62 scoring matrix, a gap length penalty of 10, and a gap penalty of 0.05.

As used herein, a cysteine-rich domain of a TANGO 223 polypeptide includes about 60-140 amino acid residues, preferably about 70-130 amino acid residues, more preferably about 80-120 amino acid residues, and most preferably about 95-105 amino acid residues of SEQ ID NO:48. Typically, a cysteine-rich domain includes a cluster of about 5-25 cysteine residues conserved in TANGO 223 protein family members, more preferably about 10-18 cysteine residues, and still more preferably about 15 cysteine residues. In addition, a cysteine-rich domain includes at least the following consensus sequence: C-Xaa(n1)-C-Xaa(n1)-C-Xaa(n4)-C-Xaa(n1)-C-Xaa(n1)-C-Xaa(n2)-C-Xaa(n1)-C-Xaa(n3)-C-Xaa(n4)-C-Xaa(n2)-C-Xaa(n1)-C-Xaa(n3)-C-Xaa(n1)-C-Xaa(n3)-C, wherein C is a cysteine residue, Xaa is any amino acid, n1 is about 2-12 amino acid residues, more preferably about 3-10 amino acid residues, and more preferably 4-8 amino acid residues in length, n2 is about 5-15 amino acid residues, more preferably 7-12 amino acid residues, and more preferably 9-10 amino acid residues in length, n3 is about 6-22 amino acid residues, more preferably about 8-20 amino acid residues, and more preferably 10-17 amino acid residues in length, and n4 is about 1-7 amino acid residues, more preferably 1-5 amino acid residues, and more preferably 2-3 amino acid residues in length. In one embodiment, a TANGO 223 family member includes a cysteine-rich domain having an amino acid sequence that is at least about 55%, preferably at least about 65%, more preferably at least about 75%, yet more preferably at least about 85%, and most preferably at least about 95% identical to amino acids 68 to 178, which is the cysteine-rich domain of TANGO 223. In another embodiment, a TANGO 223 family member includes a cysteine-rich domain having an amino acid sequence that is at least about 55%, preferably at least about 65%, more preferably at least about 75%, yet more preferably at least about 85%, and most preferably at least about 95% identical to amino acids 68 to 178, includes a conserved cluster of 15 cysteine residues, and a cysteine-rich domain consensus sequence as described herein. In yet another embodiment, a TANGO 223 family member includes a cysteine-rich domain having an amino acid sequence that is at least 55%, preferably at least about 65%, more preferably at least about 75%, yet more preferably at least about 85%, and most preferably at least about 95% identical to amino acids 68 to 178, includes a conserved cluster of 15 cysteine residues, a cysteine-rich consensus sequence as described herein, and has at least one TANGO 223 biological activity as described herein.

In a preferred embodiment, a TANGO 223 family member has the amino acid sequence wherein the cluster of conserved cysteine residues is located within amino acid residues 68 to 178 (at positions 68, 74, 81, 84, 90, 100, 108, 125, 128, 138, 144, 149, 158, 166, and 178), and the cysteine-rich domain consensus sequence is located at amino acid residue 68 to amino acid residue 178 of SEQ ID NO:48.

### Uses of TANGO 223 Nucleic Acids, Polypeptides and Modulators Thereof

TANGO 223 polypeptides, nucleic acids, and modulators thereof, can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which TANGO 223 is expressed include, for example, heart, brain, liver, kidney, testis, prostate, ovary, small intestine, colon, and peripheral blood leukocytes.

In one example, TANGO 223 polypeptides, nucleic acids, or modulators thereof, can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

In another example, TANGO 223 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 223 polypeptides, nucleic acids, or modulators thereof, can be used to treat hepatic disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g. hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 223 polypeptides, nucleic acids, or modulators thereof, can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 223 polypeptides, nucleic acids, or modulators thereof, can be used to treat testicular disorders, such as unilateral testicular enlargement (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

In another example, TANGO 223 polypeptides, nucleic acids, or modulators thereof, can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

In another example, TANGO 223 polypeptides, nucleic acids, or modulators thereof, can be used to treat ovarian disorders, such as non-neoplastic cysts (e.g., follicular and luteal cysts and polycystic ovaries) and tumors (e.g., tumors of surface epithelium, germ cell tumors, sex cord-stromal tumors, and metastatic carcinomas.

In another example, TANGO 223 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, TANGO 223 polypeptides, nucleic acids, or modulators thereof, can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., idiopathic ulcerative colitis, pseudomembranous colitis, and lymphopathia venereum), tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas).

In another example, TANGO 223 polypeptides, nucleic acids, or modulators thereof, can be used to treat leukocytic disorders, such as leukopenias (e.g., neutropenia, monocytopenia, lymphopenia, and granulocytopenia), leukocytosis (e.g., granulocytosis, lymphocytosis, eosinophilia, monocytosis, acute and chronic lymphadenitis), malignant lymphomas (e.g., Non-Hodgkin's lymphomas, Hodgkin's lymphomas, leukemias, agnogenic myeloid metaplasia, multiple myeloma, plasmacytoma, Waldenstrom's macroglobulinemia, heavy-chain disease, monoclonal gammopathy, histiocytoses, eosinophilic granuloma, and angioimmunoblastic lymphadenopathy).

### TANGO 216

In one aspect, the present invention is based on the discovery of cDNA molecules which encode a novel family of proteins having a von Willebrand factor (vWF) A domain, referred to herein as TANGO 216 proteins. Described herein are human TANGO 216, and mouse TANGO 216 nucleic acid molecules and the corresponding polypeptides which the nucleic acid molecules encode.

For example, the TANGO 216 proteins of the invention include a domain which bears sequence identity to a vWF A domain. Proteins having such a domain are involved in biological processes controlled by specific, often adhesive, molecular interactions. The vWF A domain mediates binding to proteins and sugars. Proteins having vWF A domains may interact through homophilic interactions between vWF A domains. Thus, included within the scope of the invention are TANGO 216 proteins having a vWF A domain. As used herein, a vWF A domain refers to an amino acid sequence of about 150 to 190, preferably about 155 to 185, 160 to 180, and more preferably about 170 amino acids in length. Conserved amino acid motifs, referred to herein as “consensus patterns” or “signature patterns”, can be used to identify TANGO216 family members. For example, the following signature pattern can be used to identify TANGO 216 family members: D-x (2)-F-[ILV]-x-D-x-S-x (2, 3)-[ILV]-x (10, 12)-F. TANGO 216 has such a signature pattern at about amino acids 44 to 169 of SEQ ID NO:51.

The vWF A domain consensus sequence is also available from the HMMer version 2.0 software as Accession Number PF00092. Software for HMM-based profiles is available from http://www.csc.ucsc.edu/research/compbio/sam.html and from http://genome.wustl.edu/eddy/hmmer.html. A vWF A domain of TANGO 216 extends, for example, from about amino acids 44 to 213.

Also included within the scope of the present invention are TANGO 216 proteins having a signal sequence.

In certain embodiments, a TANGO 216 family member has the amino acid sequence, and the signal sequence is located at amino acids 1 to 31, 1 to 32, 1 to 33, 1 to 34 or 1 to 35. In such embodiments of the invention, the domains and the mature protein resulting from cleavage of such signal peptides are also included herein. For example, the cleavage of a signal sequence consisting of amino acids 1 to 33 of SEQ ID NO:52 results in a mature TANGO 216 protein corresponding to amino acids 34 to 488 of SEQ ID NO:52. The signal sequence is normally cleaved during processing of the mature protein.

The present invention also includes TANGO 216 proteins having a transmembrane domain. An example of a transmembrane domain includes from about amino acids 318 to 345 of SEQ ID NO:52.

In one embodiment, a TANGO 216 protein of the invention includes a vWF A domain. In another embodiment, a TANGO 216 protein of the invention includes a vWF A domain, and a signal sequence. In another embodiment, a TANGO 216 protein of the invention includes a vWF A domain, a extracellular domain, and a signal sequence. In another embodiment, a TANGO 216 protein of the invention includes a vWF A domain, and an extracellular domain. In another embodiment, a TANGO 216 protein of the invention includes a vWF A domain, an extracellular domain, and a transmembrane domain. In another embodiment, a TANGO 216 protein of the invention includes a vWF A domain, an extracellular domain, a transmembrane domain, and a cytoplasmic domain.

### Human TANGO 216

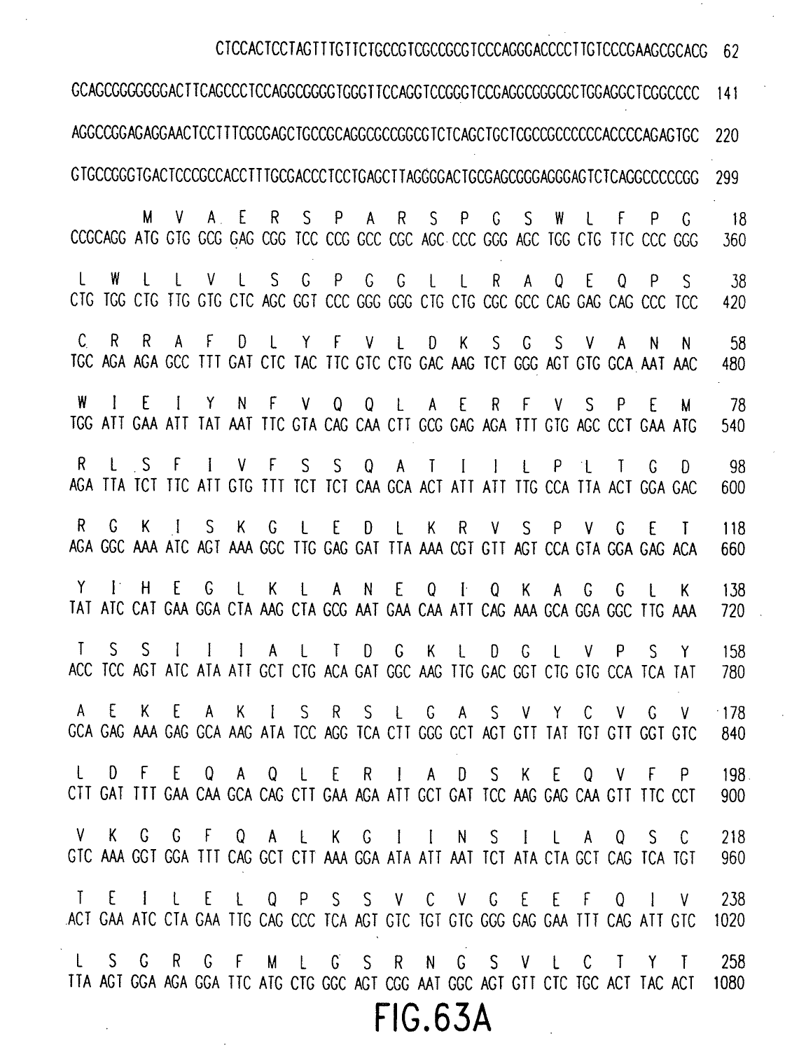

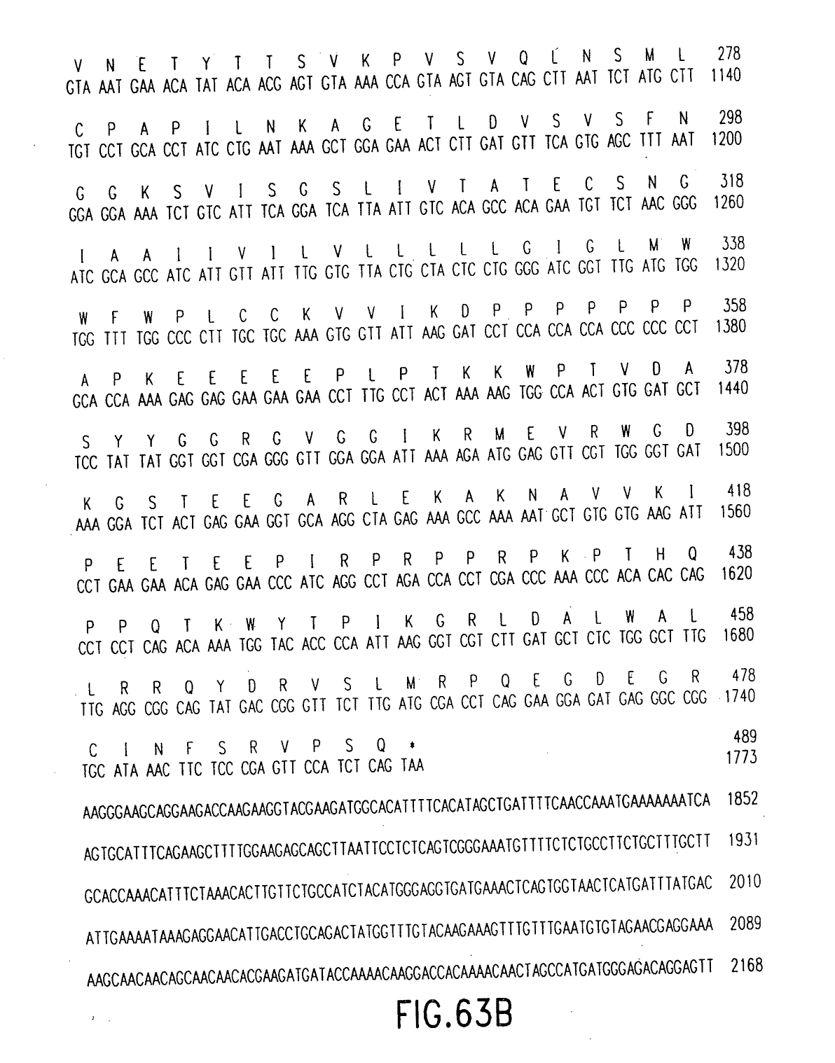

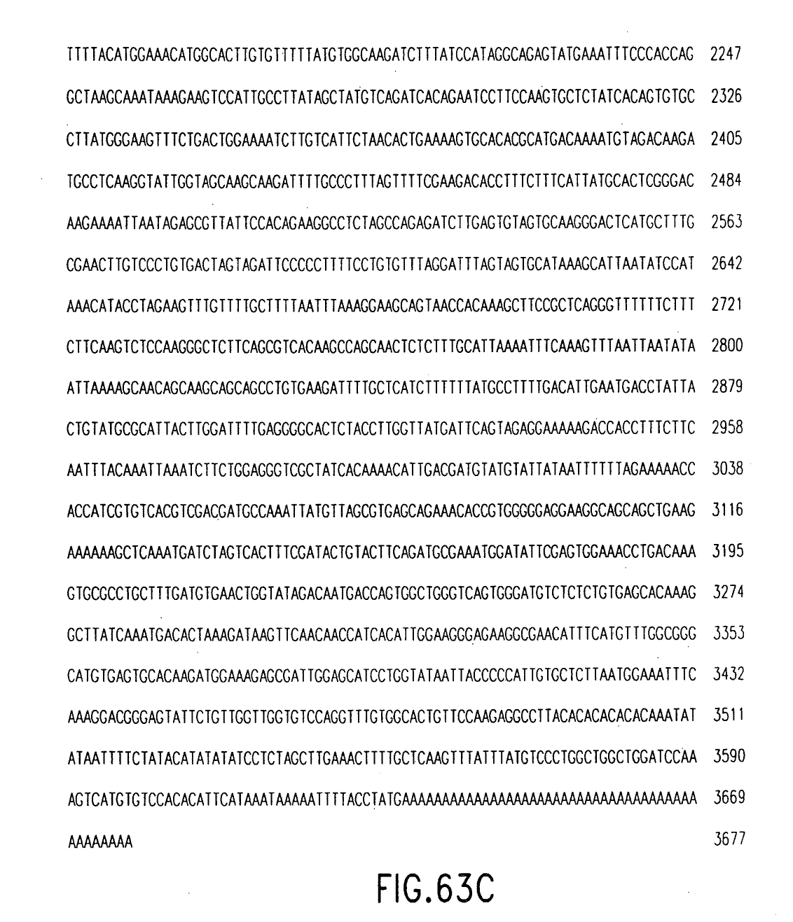

The cDNA encoding human TANGO 216 was isolated by screening for cDNAs which encode a potential signal sequence. Briefly, a clone encoding TANGO 216 was isolated through high throughput screening of a prostate stroma cell library. The human TANGO 216 clone includes a 3677 nucleotide cDNA (FIG. 63A-63C; SEQ ID NO:51). The open reading frame of this cDNA (nucleotides 307 to 1770 of SEQ ID NO:51), encodes a 488 amino acid transmembrane protein depicted in of SEQ ID NO:52.

In another embodiment, a human TANGO 216 clone comprises a 4350 nucleotide cDNA. The open reading frame of this cDNA comprises nucleotides 353 to 1819, and encodes a the human TANGO 216 transmembrane protein comprising 488 amino acids.

In one embodiment of a nucleotide sequence of human TANGO 216, the nucleotide at position 318 is a guanine (G). In this embodiment, the amino acid at position 12 is glutamate (E). In another embodiment of a nucleotide sequence of human TANGO 216, the nucleotide at position 318 is a cytosine (C). In this embodiment, the amino acid at position 12 is aspartate (D). In another embodiment of a nucleotide sequence of human TANGO 216, the nucleotide at position 411 is a guanine (G). In this embodiment, the amino acid at position 35 is a glutamate (E). In another embodiment of a nucleotide sequence of human TANGO 216, the nucleotide at position 411 is a cytosine (C). In this embodiment, the amino acid at position 35 is aspartate (D). In another embodiment of a nucleotide sequence of human TANGO 216, the nucleotide at position 489 is an adenine (A). In this embodiment, the amino acid at position 61 is a glutamate (E). In another embodiment of a nucleotide sequence of human TANGO 216, the nucleotide at position 489 is a cytosine (C). In this embodiment, the amino acid at position 61 is aspartate (D).

The signal peptide prediction program SIGNALP (Nielsen et al. (1997) Protein Engineering 10:1-6) predicted that human TANGO 216 includes a 33 amino acid signal peptide (amino acids 1 to about amino acid 33 of SEQ ID NO:52) preceding the mature TANGO 216 protein (corresponding to about amino acid 34 to amino acid 488 of SEQ ID NO:52). The presence of a methionine residue at positions 78, 245, 277, 337, 392, and 369 indicate that there can be alternative forms of human TANGO 216 of 411 amino acids, 244 amino acids, 212 amino acids, 152 amino acids, 97 amino acids, and 120 amino acids of SEQ ID NO:52, respectively.

In one embodiment, human TANGO 216 includes extracellular domains (about amino acids 34 to 79 and 342 to 488), transmembrane (TM) domains (amino acids 80-97 and 318 to 341 of SEQ ID NO:52); and a cytoplasmic domain (amino acids 98 to 317 of SEQ ID NO:52). The cytoplasmic domain is very rich in proline and glutamic acid residues. These residues represent 27% of the residues in the cytoplasmic domain of human TANGO 216.

Alternatively, in another embodiment, a human TANGO 216 protein contains an extracellular domain at amino acid residues 98 to 317, transmembrane (TM) domains (amino acids 80-97 and 318 to 341, and cytoplasmic domains at amino acid residues 1 to 79 and 342-488 of SEQ ID NO:52).

Another embodiment of the invention includes isolated nucleic acid molecules comprising a polynucleotide having a nucleotide sequence encoding the polypeptide having the human TANGO 216 amino acids, but lacking the N-terminal methionine residue. In this embodiment, the nucleotide sequence of human TANGO 216, nucleotides 310-1770, encodes the human TANGO 216 amino acid sequence from amino acids 2-488 of SEQ. ID NO:52.

Human TANGO 216 includes a vWF A domain from about amino acids 44 to 213 of SEQ ID NO:52.

Human TANGO 216 protein, including the signal sequence, has a molecular weight of 53.6 kDa prior to post-translational modification. Human TANGO 216 protein has a molecular weight of 50.0 kDa after cleavage of the 33 amino acid signal peptide.

A clone, EpT216, which encodes human TANGO 216 was deposited with the American Type Culture Collection (ATCC®, 10801 University Boulevard, Manassas, Va. 20110-2209) on Mar. 26, 1999, and was assigned Accession Number 207176. This deposit will be maintained under the terms of the Budapest Treaty on the International Recognition of the Deposit of Microorganisms for the Purposes of Patent Procedure. This deposit was made merely as a convenience to those of skill in the art and is not an admission that a deposit is required under 35 U.S.C. §112.

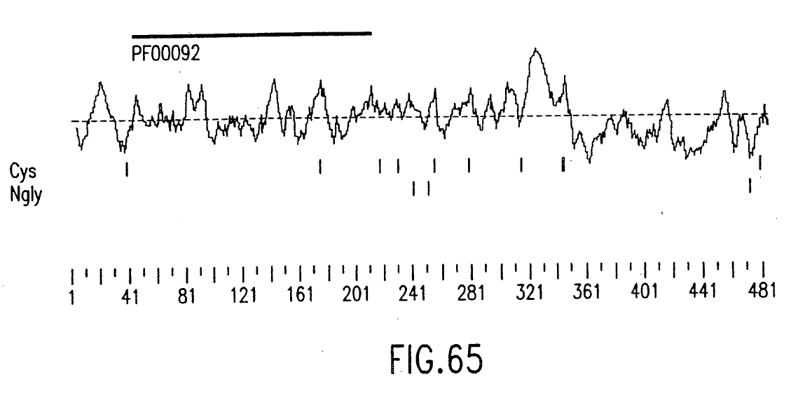

FIG. 65 depicts a hydropathy plot of human TANGO 216. As shown in the hydropathy plot, the hydrophobic region at the beginning of the plot which corresponds to about amino acids 1 to 33 of SEQ ID NO:52 is the signal sequence of TANGO 216.

Northern analysis of human TANGO 216 mRNA expression revealed the presence of an approximately 3.8 kb transcript and an approximately 4.3 kb transcript that are expressed in a range of tissues including lung, liver, skeletal muscle, kidney, and pancreas, with highest expression in heart and placenta. The two transcripts likely represent alternative poly A site usage.

The human gene for TANGO 216 was mapped on radiation hybrid panels to the long arm of chromosome 4, in the region q11-13. Flanking markers for this region are GCT14E02 and jktbp-rs2. The JPD (periodontitis, juvenile), and DGI1(dentinogenesis imperfecta) loci also map to this region of the human chromosome. The GRO1 (FRO1 oncogene), ALB (albumin), IL8 (interleukin 8), HTN (histatin), and DCK (deoxycytidine kinase) genes also map to this region of the human chromosome. This region is syntenic to mouse chromosome 5. The rs (recessive spotting) locus also maps to this region of the mouse chromosome. The step (sulfotransferase), areg (amphiregulin), btc (betacellulin), mc (marcel), alb1 (albumin 1), and afp (alpha fetoprotein) genes also map to this region of the mouse chromosome.

### Mouse TANGO 216

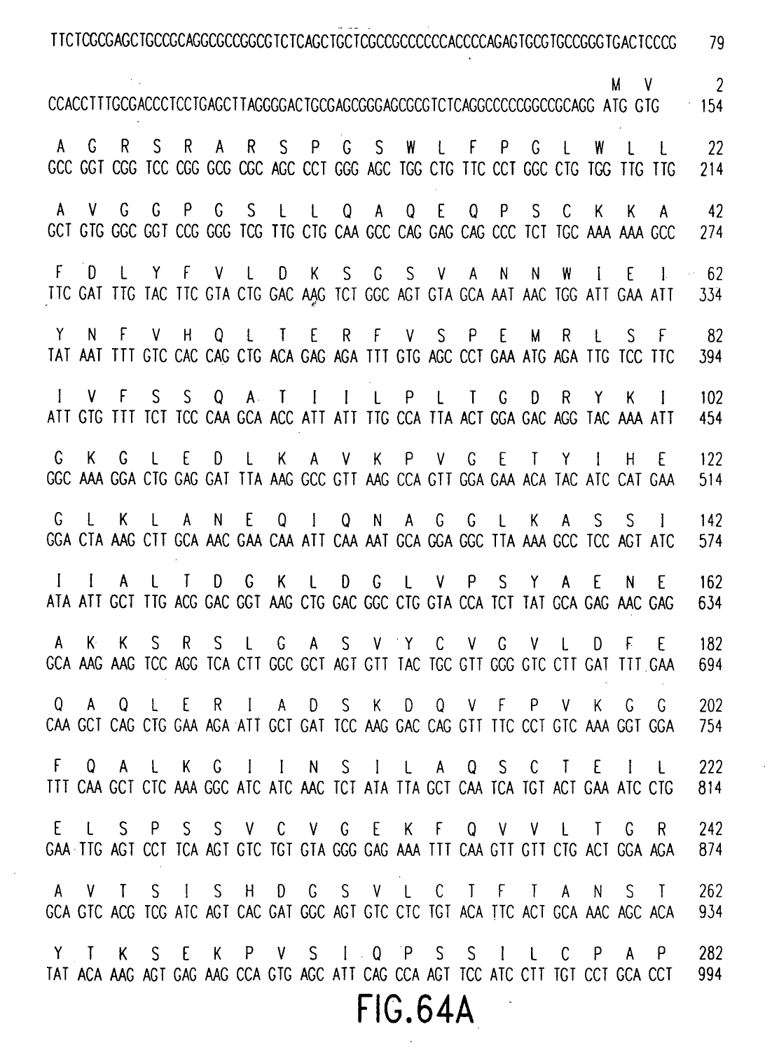

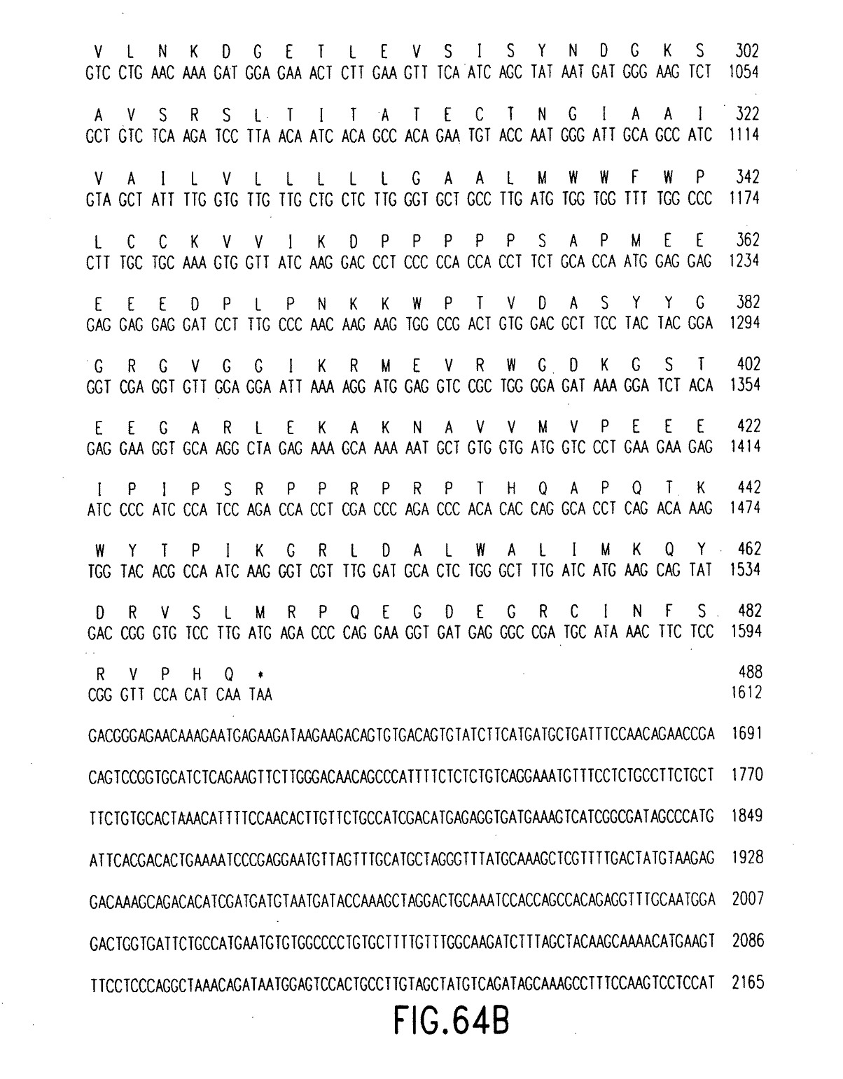

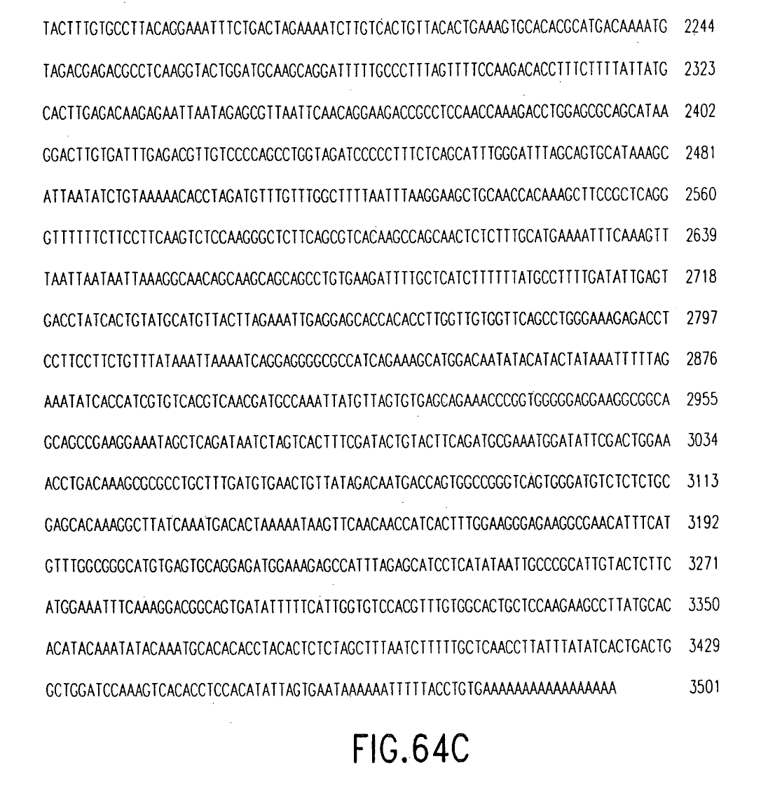

A mouse homolog of human TANGO 216 was identified. A cDNA encoding mouse TANGO 216 was identified by analyzing the sequences of clones present in a mouse bone marrow cDNA library. This analysis led to the identification of a clone, jtmMa005g09, encoding mouse TANGO 216. The mouse TANGO 216 cDNA of this clone is 3501 nucleotides long (FIG. 64A-64C; SEQ ID NO:53). The open reading frame of this cDNA (nucleotides 149 to 1609 of SEQ ID NO:53) encodes the 487 amino acid protein depicted in SEQ ID NO:54.

In another embodiment, a mouse TANGO 216 clone comprises a 3647 nucleotide cDNA. The open reading frame of this cDNA comprises nucleotides 32 to 469, and encodes a mouse TANGO 216 transmembrane protein comprising the 146 amino acids.

In one embodiment, mouse TANGO 216 includes extracellular domains (about amino acids 34 to 79 and 342 to 487, transmembrane (TM) domains (amino acids 80-97 and 318 to 341 of SEQ ID NO:54); and a cytoplasmic domain (amino acids 98 to 317 of SEQ ID NO:54). The cytoplasmic domain is very rich in proline and glutamic acid residues. These residues represent 27% of the residues in the cytoplasmic domain of human TANGO 216. Alternatively, in another embodiment, a mouse TANGO 216 protein contains an extracellular domain at amino acid residues 98 to 317, transmembrane (TM) domains (amino acids 80-97 and 318 to 341, and cytoplasmic domains at amino acid residues 1 to 79 and 342-487 of SEQ ID NO:54.

The signal peptide prediction program SIGNALP (Nielsen et al. (1997) Protein Engineering 10:1-6) predicted that mouse TANGO 216 includes a 33 amino acid signal peptide (amino acids 1 to about amino acid 336 of SEQ ID NO:54) preceding the mature TANGO 216 protein (corresponding to about amino acid 34 to amino acid 487 of SEQ ID NO:54). The presence of a methionine residue at positions 78, 337, 360, 392, 417, 459, and 468 of SEQ ID NO:54 indicate that there can be alternative forms of mouse TANGO 216 of 410 amino acids, 151 amino acids, 128 amino acids, 96 amino acids, 71 amino acids, 29 amino acids, and 20 amino acids of SEQ ID NO:54, respectively.

In one embodiment of a nucleotide sequence of mouse TANGO 216 the nucleotide at position 253 is a guanine (G). In this embodiment, the amino acid at position 35 is glutamate (E). In another embodiment of a nucleotide sequence of mouse TANGO 216, the nucleotide at position 253 is a cytosine (C). In this embodiment, the amino acid at position is aspartate (D). In another embodiment of a nucleotide sequence of mouse TANGO 216, the nucleotide at position 331 is an adenine (A). In this embodiment, the amino acid at position 61 is a glutamate (E). In another embodiment of a nucleotide sequence of mouse TANGO 216, the nucleotide at position 331 is a cytosine (C). In this embodiment, the amino acid at position 61 is aspartate (D). In another embodiment of a nucleotide sequence of mouse TANGO 216, the nucleotide at position 371 is a guanine (G). In this embodiment, the amino acid at position 71 is a glutamate (E). In another embodiment of a nucleotide sequence of mouse TANGO 216, the nucleotide at position 371 is a cytosine (C). In this embodiment, the amino acid at position 71 is aspartate (D).

Another embodiment of the invention includes isolated nucleic acid molecules comprising a polynucleotide having a nucleotide sequence encoding the polypeptide having the mouse TANGO 216 amino acid sequence, but lacking the N-terminal methionine residue. In this embodiment, the nucleotide sequence of mouse TANGO 216, nucleotides 152-1609, encodes the mouse TANGO 216 amino acid sequence comprising amino acids 2-487 of SEQ ID NO:54.

Mouse TANGO 216 includes a vWF A domain from about amino acids 44 to 213 of SEQ ID NO:54.

Mouse TANGO 216 protein, including the signal sequence, has a molecular weight of 53.2 kDa prior to post-translational modification. Mouse TANGO 216 protein has a molecular weight of 49.8 kDa after cleavage of the 33 amino acid signal peptide.

In situ tissue screening was performed on mouse adult and embryonic tissue to analyze the expression of mouse TANGO 216 mRNA. In the case of adult expression, a low level ubiquitous signal was detected in the spleen and stomach. A weak, ubiquitous signal was detected in the thymus. A ubiquitous signal was detected in the liver, submandibular salivary gland, heart, colon, and in the cortical region of the adrenal gland. A multifocal pattern was detected in the lung and in the decidua of the placenta. A signal was apparent in the villi of the small intestine. No signal was detected in the following tissues: brain, spinal cord, eye, brown fat, white fat, pancreas, skeletal muscle, bladder, kidney, and lung.

In the case of embryonic expression, expression was seen in a number of tissues. At E13.5, strong signals were detected in the developing spinal column, heart, and tongue. Meckelis cartilage was also apparent. Limb expression is not readily apparent. Low level signal was also seen throughout the gut region including but not restricted to lung, liver, and intestines. Signal is noticeably absent from the developing CNS except for the areas of the brain surrounding the lateral ventricals and mesencephalic vesicle. At E14.5, developing spinal column and sternum, heart, tongue, and Meckelis cartilage continued to have strong signal. Signal from the heart and tongue was ubiqutious. In the brain, the diencephalon had the strongest signal with the areas surrounding the ventricles still being positive. At E15.5, signal was seen in the previously stated regions and was readily seen in the primordium of the basisphenoid bone and primordium of the nasal bone. At E16.5, signal was seen in the previously stated regions, primordium of the basisphenoid bone. At E18.5, the strongest signal was obtained in the developing bone and cartilage areas. Signal from the heart was diminished in strength and now equal to that seen in the rest of the gut region. At P1.5, signal was still strong in the spinal column and nasal septum. Signal was absent from the CNS except for faint signal in the region of the developing cerebellum. Signal is otherwise low and ubiquitous except for heart, small intestine, and stomach which have a slightly higher signal. The highest expressing tissue was the capsule of the kidney which was seen at E14.5 and continues to P1.5.

Human and mouse TANGO 216 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 84.8%. The human and mouse TANGO 216 full length cDNAs are 84.4% identical, as assessed using the same software and parameters as indicated (without the BLOSUM 62 scoring matrix). In the respective ORFs, calculated in the same fashion as the full length cDNAs, human and mouse TANGO 216 are 84% identical.

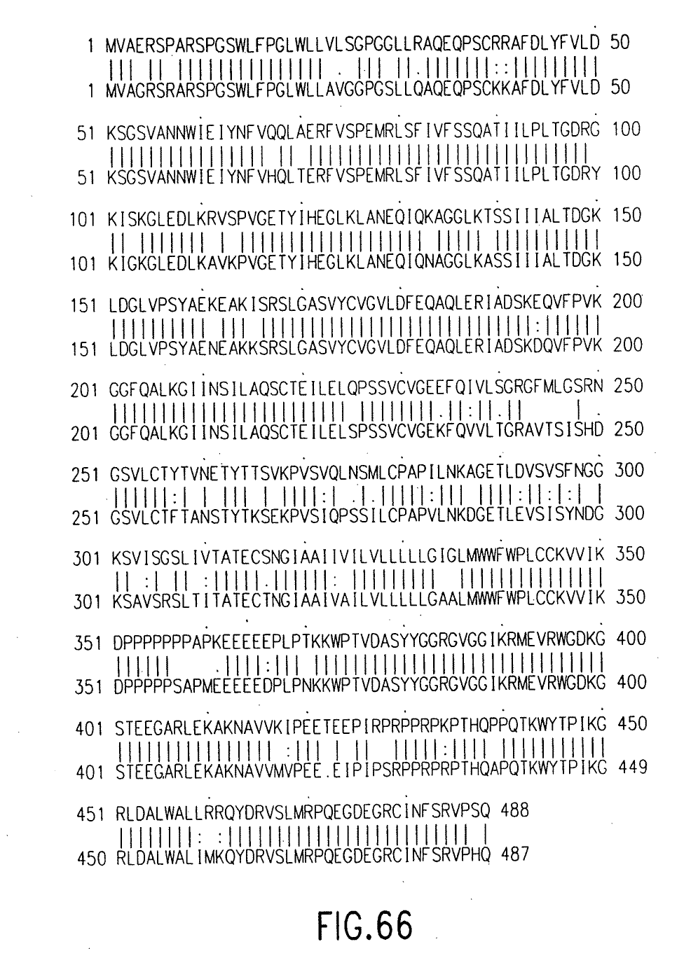

FIG. 66 depicts the alignment of the amino acid sequence of human TANGO 216 and mouse TANGO 216. In this alignment, a (|) between the two sequences indicates an exact match. The depicted alignment of the amino acid sequence of human TANGO 216 (SEQ ID NO:52) and mouse TANGO 216 (SEQ ID NO:54) over 146 amino acids of mouse TANGO 216, indicate a percent identity of approximately 65-68%.

### Uses of TANGO 216 Nucleic Acids, Polypeptides, and Modulators Thereof

The TANGO 216 proteins of the invention include a vWF A domain. Accordingly, TANGO 216 proteins likely function in a similar manner as other proteins which include a vWF A domain, including von Willebrand factor, a large multimeric protein found in platelets, endothelial cells, and plasma. Thus, TANGO 216 modulators can be used to treat any von Willebrand factor-associated disorders and modulate normal von Willebrand factor functions.

As discussed above, the vWF domain of TANGO 216 is involved in cellular adhesion and interaction with extracellular matrix (ECM) components. Proteins of the type A module superfamily which incorporate a vWF domain participate in multiple ECM and cell/ECM interactions. For example, proteins having a vWF domain have been found to play a role in cellular adhesion, migration, homing, pattern formation and/or signal transduction after interaction with several different ligands (Colombatti et al. (1993) Matrix 13:297-306).

Similarly, the TANGO 216 proteins of the invention likely play a role in various extracellular matrix interactions, e.g., matrix binding, and/or cellular adhesion. Thus, a TANGO 216 activity is at least one or more of the following activities: 1) regulation of extracellular matrix structuring; 2) modulation of cellular adhesion, either in vitro or in vivo; 3) regulation of cell trafficking and/or migration. Accordingly, the TANGO 216 proteins, nucleic acid molecules and/or modulators can be used to modulate cellular interactions such as cell-cell and/or cell-matrix interactions and thus, to treat disorders associated with abnormal cellular interactions.

TANGO 216 polypeptides, nucleic acids and/or modulators thereof can also be used to modulate cell adhesion in proliferative disorders, such as cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenstrom's macroglobulinemia.

As TANGO 216 was originally isolated from a bone marrow library, TANGO 216 nucleic acids, proteins, and modulators thereof can be used to modulate the proliferation, differentiation, and/or function of cells that appear in the bone marrow, e.g., stem cells (e.g., hematopoietic stem cells), and blood cells, e.g., erythrocytes, platelets, and leukocytes. Thus TANGO 269 nucleic acids, proteins, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

As TANGO 216 exhibits expression in the embryonic lung, TANGO 216 polypeptides, nucleic acids, or modulators thereof, can be used to treat pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

As TANGO 216 exhibits expression in the small intestine, TANGO 216 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

As TANGO 216 exhibits expression in the spleen, TANGO 216 nucleic acids, proteins, and modulators thereof can be used to modulate the proliferation, diffirentiation, and/or function of cells that form the spleen, e.g., cells of the splenic connective tissue, e.g., splenic smooth muscle cells and/or endothelial cells of the splenic blood vessels. TANGO 216 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 216 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 216 is expressed in the kidney, the TANGO 216 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such can be used to treat or modulate renal (kidney) disorders, such as glomerular diseases (e.g. acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As TANGO 216 exhibits expression in the heart, TANGO 216 polypeptides, nucleic acids, or modulators thereof, can be used to treat cardiovascular disorders as described herein.

As TANGO 216 exhibits expression in bone structures, TANGO 216 nucleic acids, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarhritis, and bone wearing.

The extracellular region of TANGO 216 has significant similarity to TANGO 197, a secreted protein. TANGO 197 has a vWF A domain and may interact with TANGO 216.

TANGO 216 likely plays a role in the regulation of binding of cells in circulation to the endothelial substrate. Thus, TANGO-216 may regulate proper flow of cells in the heart, vasculature, and placenta. Accordingly, the TANGO 216 proteins, nucleic acids and/or modulators of the invention are useful modulators of interactions between cells in circulation and endothelial substrate which can be used to treat disorders of such interactions.

### Human TANGO 261

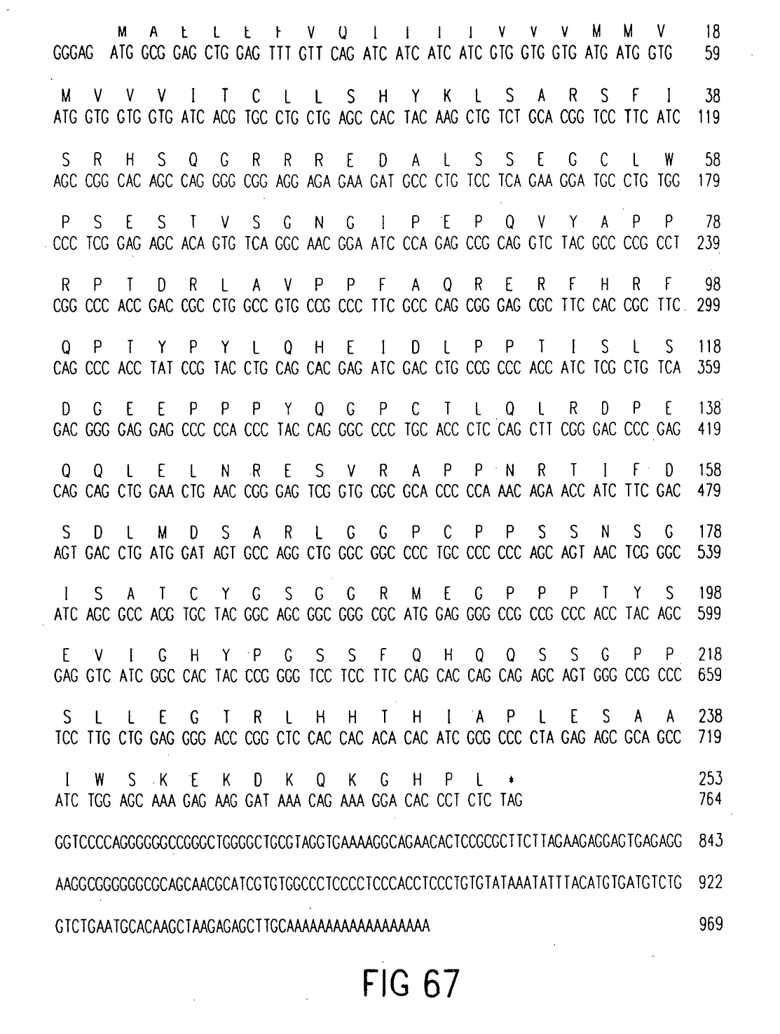

A cDNA clone, jthda088f09, encoding full length human TANGO 261 was identified by screening a stimulated human smooth muscle cell library by EST analysis. Another cDNA clone, jthkf124b08, encoding full length hunan TANGO 261 was identified by screening a stimulated keratinocyte cell library by EST analysis. The 969 nucleotide human TANGO 261 sequence (FIG. 67; SEQ ID NO:55) includes a open reading frame which extends from nucleotide 6 to nucleotide 761 of SEQ ID NO:55 and encodes a 252 amino acid secreted protein (SEQ ID NO:56).

In another embodiment, a human TANGO 261 clone includes comprises a 1942 nucleotide cDNA. The open reading frame of this cDNA comprises nucleotides 146 to 904, and encodes a transmembrane protein comprising the 252 amino acid sequence.

In one embodiment of a nucleotide sequence of human TANGO 261 the nucleotide at position 14 is a guanine (G). In this embodiment, the amino acid at position 3 is glutamate (E). In another embodiment of a nucleotide sequence of human TANGO 261, the nucleotide at position 14 is a cytosine (C). In this embodiment, the amino acid at position 3 is aspartate (D) In another embodiment of a nucleotide sequence of human TANGO 261, the nucleotide at position 149 is an adenine (A). In this embodiment, the amino acid at position 48 is a glutamate (E). In another embodiment of a nucleotide sequence of human TANGO 261, the nucleotide at position 149 is a cytosine (C). In this embodiment, the amino acid at position 48 is aspartate (D). In another embodiment of a nucleotide sequence of human TANGO 261, the nucleotide at position 167 is an adenine (A). In this embodiment, the amino acid at position 54 is a glutamate (E). In another embodiment of a nucleotide sequence of human TANGO 261, the nucleotide at position 167 is a cytosine (C). In this embodiment, the amino acid at position 54 is aspartate (D).

In certain embodiments, a TANGO 261 family member has the amino acid sequence, and the signal sequence is located at amino acids 1 to 26, 1 to 27, 1 to 28, 1 to 29 or 1 to 30. In such embodiments of the invention, the domains and the mature protein resulting from cleavage of such signal peptides are also included herein. For example, the cleavage of a signal sequence consisting of amino acids 1 to 28 (SEQ ID NO:56) results in a mature TANGO 261 protein corresponding to amino acids 29 to 252 of SEQ ID NO:56. The signal sequence is normally cleaved during processing of the mature protein. Thus, in one embodiment, a TANGO 261 protein includes a signal sequence and is secreted.

Another embodiment of the invention includes isolated nucleic acid molecules comprising a polynucleotide having a nucleotide sequence encoding the polypeptide having the human TANGO 261 amino acid sequence but lacking the N-terminal methionine residue. In this embodiment, the nucleotide sequence of human TANGO 261, nucleotides 9-761 of SEQ ID NO:55, and encodes the human TANGO 261 amino acid sequence comprising amino acids 2-252 of SEQ ID NO:56.

Human TANGO 261 includes a signal sequence (amino acid 1 to about amino acid 28 of SEQ ID NO:56) preceding the mature protein (about amino acid 29 to amino acid 252 of SEQ ID NO:56). The presence of a methionine residue at positions 16, 17, 19, 162, and 190 indicate that there can be alternative forms of human TANGO 261 of 237 amino acids, 236 amino acids, 234 amino acids, 91 amino acids, and 63 amino acids of SEQ ID NO:56, respectively.

Human TANGO 261 protein, including the signal sequence, has a molecular weight of 27.9 kD prior to post-translational modification. Mature human TANGO 261 protein has a molecular weight of 24.8 kD prior to post-translational modification.

A clone, EpT261, which encodes human TANGO 261 was deposited with the American Type Culture Collection (ATCC®, 10801 University Boulevard, Manassas, Va. 20110-2209) on Mar. 26, 1999, and assigned Accession Number 207176. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

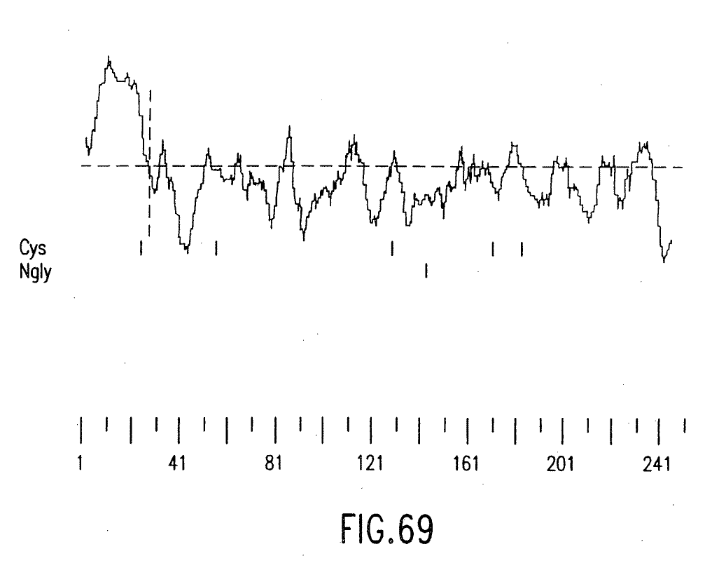

FIG. 69 depicts a hydropathy plot of human TANGO 261. As shown in the hydropathy plot, the hydrophobic region of the plot which corresponds to amino acid 1 to about amino acid 28 is the signal sequence of TANGO 261.

Northern analysis of human TANGO 261 mRNA expression revealed the presence of an approximately 2.6 kb transcript and an approximately 6.0 kb transcript that are expressed in a range of tissues including lung, liver, kidney, and placenta, with highest expression in heart and skeletal muscle. No expression was observed in colon, thymus, peripheral blood leukocytes, and spleen. The two transcripts likely represent alternative poly A site usage.

Human TANGO 261 is likely expressed in prostate epithelium, prostate smooth muscle, bone, and brain, based on the origin of ESTs.

The human gene for TANGO 261 was mapped on radiation hybrid panels to the long arm of chromosome 20, in the region ql3.2-13.3. Flanking markers for this region are WI-3773 and AFMA202YB9. The EEGV1 (electroencephalographic variant pattern 1) and PHP1B (pseudohypoparathyroidism) loci also map to this region of the human chromosome. The MC3R (melanocortin 3 receptor), EDN3 (endothelin 3), ADA (adenosine deaminase), and OQTL (obesity QTL) genes also map to this region of the human chromosome. This region is syntenic to mouse chromosome 2. The fc (flecking) and ra (ragged) loci also map to this region of the mouse chromosome. The mc3r (melanocortin 3 receptor), fc (flecking), ra (ragged), and ntsr (neurotensin receptor) genes also map to this region of the mouse chromosome.

The open reading frame of human TANGO 261 bears significant similarity to the open reading frame of human clone 22 mRNA, alternative splice variant beta 2 (GenBank Accession Number AF009427; Sanders et al. (1997) Am J. Med. Genet. 74:140-9), a gene which has brain-specific expression, produces an 8 kb mRNA encoding a 230 amino acid protein, and maps near the candidate region for bipolar affective disorder on chromosome 18. Human TANGO 261 protein and the protein encoded by clone 22 mRNA, alternative splice variant beta 2 are approximately 70% identical. However, human TANGO 261 does not appear to be brain specific.

### Mouse TANGO 261

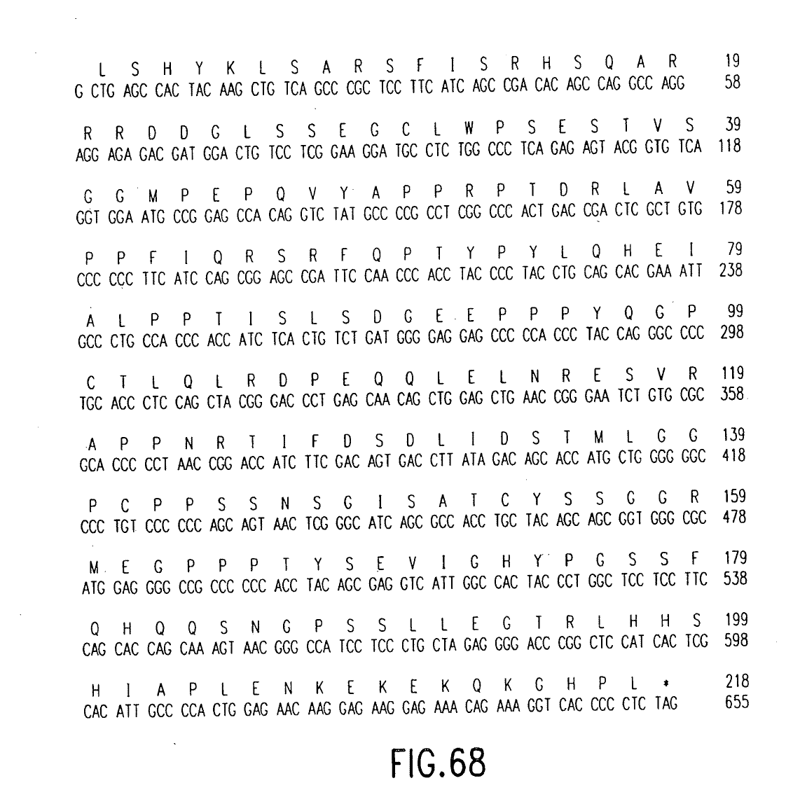

A mouse homolog of human TANGO 261 was identified. A cDNA encoding mouse TANGO 261 was identified by analyzing the sequences of clones present in a mouse microglial cell cDNA library. This analysis led to the identification of a clone, jtmxa004g06, encoding mouse TANGO 261. The mouse TANGO 261 cDNA of this clone is 1713 nucleotides long (FIG. 68; SEQ ID NO:57). The open reading frame of this cDNA (nucleotides 2 to 652 of SEQ ID NO:57) encodes a protein comprising a 217 amino acid protein (SEQ ID NO:58).

In another embodiment, a mouse TANGO 261 clone includes comprises a 484 nucleotide cDNA. The open reading frame of this cDNA comprises nucleotides 3 to 413, and encodes a transmembrane protein comprising the 137 amino acid sequence.

The predicted molecular weight of a mouse TANGO 261 protein without post-translational modifications is 23.9 kDa. The presence of a methionine residue at positions 42, 136, and 160 indicate that there can be alternative forms of mouse TANGO 261 comprising 176 amino acids, 82 amino acids, and 58 amino acids of SEQ ID NO:58, respectively.

In one embodiment of a nucleotide sequence of mouse TANGO 261 the nucleotide at position 85 is an adenine (A). In this embodiment, the amino acid at position 28 is glutamate (E). In another-embodiment of a nucleotide sequence of mouse TANGO 261, the nucleotide at position 85 is a cytosine (C). In this embodiment, the amino acid at position 28 is aspartate (D). In another embodiment of a nucleotide sequence of mouse TANGO 261, the nucleotide at position 106 is a guanine (G). In this embodiment, the amino acid at position 35 is a glutamate (E). In another embodiment of a nucleotide sequence of mouse TANGO 261, the nucleotide at position 106 is a cytosine (C). In this embodiment, the amino acid at position 35 is aspartate (D). In another embodiment of a nucleotide sequence of mouse TANGO 261, the nucleotide at position 133 is a guanine (G). In this embodiment, the amino acid at position 44 is a glutamate (E). In another embodiment of a nucleotide sequence of mouse TANGO 261, the nucleotide at position 133 is a cytosine (C). In this embodiment, the amino acid at position 44 is aspartate (D).

Another embodiment of the invention includes isolated nucleic acid molecules comprising a polynucleotide having a nucleotide sequence encoding the polypeptide having the mouse TANGO 261 amino acid sequence, but lacking the N-terminal methionine residue. In this embodiment, the nucleotide sequence of mouse TANGO 261, nucleotides 5-652, encodes the mouse TANGO 261 amino acid sequence comprising amino acids 2-217 SEQ ID NO:58.

In situ tissue screening was performed on mouse adult and embryonic tissue to analyze for the expression of mouse TANGO 261 rRNA. In the case of adult expression, a signal was observed in the cortex, olfactory bulb, caudate nucleus of the brain as well as in the brain stem. A weak signal was observed in the central grey matter of the spinal cord. A signal was observed in the ganglion cell layer of the eye and harderian gland. A signal was observed in the medulla of the adrenal gland. A moderate signal was observed in the cortex of the thymus. A signal was observed in the follicles of the spleen. A weak, ubiquitous signal was detected in the kidney, brown fat, and submandibular gland. A ubiquitous signal was detected in the liver, submandibular salivary gland, heart, colon, and in the cortical region of the adrenal gland. A signal was also observed in the labyrinth zone of the placenta and the mucosal epithelium of the bladder. A signal was also observed in the ovaries. No expression was observed in white fat, stomach, heart, lung, liver, lymph node, pancreas, skeletal muscle, testes, and small intestine.

In the case of embryonic expression, expression was seen in a number of tissues. At E13.5, a signal was observed in most tissues, the most noticeable exception being the liver which had a signal near background levels. The highest signal was observed in the ventricles of the brain. At E14.5, the strongest signal was observed in the eye. Weak to moderate signal was observed almost ubiquitously throughout the embryo. At E15.5 and E16.5, a strong signal was observed in the cortical region of the brain and the large vessels of the heart, descending aorta, and vessels associated with the umbilical cord. A moderate, ubiquitous signal was seen in the lung. A weak to moderate signal was observed in most other regions of the embryo. At E18.5, a very strong signal was observed in the eye, specifically the developing retina A strong signal was also seen in the large vessels of the heart, descending aorta, brown fat and submaxillary gland. A weak signal is observed in several other regions including the brain, intestinal tract, and the bladder. At P1.5, the signal had decreased to nearly background levels in most regions. The strongest signal was associated with the developing incisor teeth and the basio bone. A weak signal is also observed in the cortical and caudate regions of the brain.

Human and mouse TANGO 261 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 92.6%. The human and mouse TANGO 261 full length cDNAs are 83.9% identical, as assessed using the same software and parameters as indicated (without the BLOSUM 62 scoring matrix). In the respective ORFs, calculated in the same fashion as the full length cDNAs, human and mouse TANGO 261 are 87.4% identical.

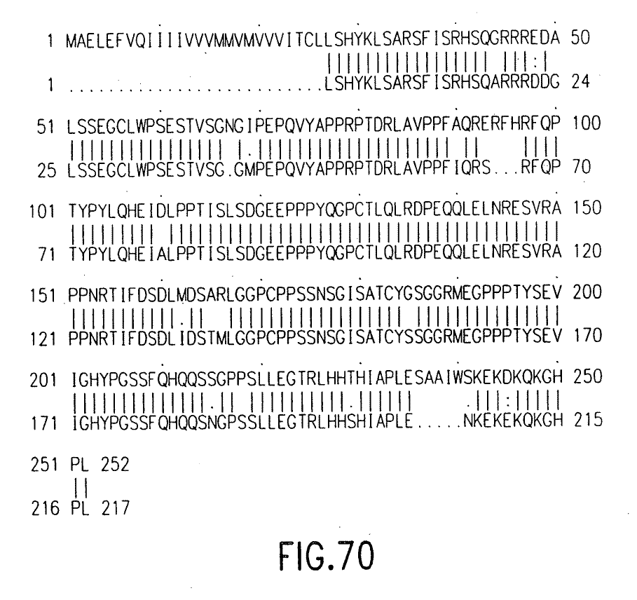

FIG. 70 depicts the alignment of the amino acid sequence of human TANGO 261 and a portion of mouse TANGO 261. In this alignment, a (|) between the two sequences indicates an exact match.

### Uses of TANGO 261 Nucleic Acids, Polypeptides, and Modulators Thereof

The TANGO 261 proteins and nucleic acid molecules of the invention have at least one “TANGO 261 activity” (also referred to herein as “TANGO 261 biological activity”). TANGO 261 activity refers to an activity exerted by a TANGO 261 protein or nucleic acid molecule on a TANGO 261 responsive cell in vivo or in vitro. Such TANGO 261 activities include at least one or more of the following activities: 1) interaction of a TANGO 261 protein with a TANGO 261-target molecule; 2) activation of a TANGO 261 target molecule; 3) modulation of cellular proliferation; 4) modulation of cellular differentiation; or 5) modulation of a signaling pathway. Thus, the TANGO 261 proteins, nucleic acids and/or modulators can be used for the treatment of a disorder characterized by aberrant TANGO 261 expression and/or an aberrant TANGO 261 activity, such as proliferative and/or differentiative disorders.

As TANGO 261 is expressed in the kidney, the TANGO 261 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such can be used to treat or modulate renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

Because TANGO 261 is expressed in the reproductive tract, particularly in the ovaries, the TANGO 261 polypeptides, nucleic acids and/or modulators thereof can be used to modulate the function, morphology, proliferation and/or differentiation of cells in the tissues in which it is expressed. For example, the TANGO 261 polypeptides, nucleic acids and/or modulators thereof can be used modulate the function, morphology, proliferation and/or differentiation of the ovaries. For example, such molecules can be used to treat or modulate disorders associated with the ovaries, including, without limitation, ovarian tumors, McCune-Albright syndrome (polyostotic fibrous dysplasia). For example, the TANGO 261 polypeptides, nucleic acids and/or modulators can be used in the treatment of infertility.

As TANGO 261 exhibits expression in the lung, TANGO 261 polypeptides, nucleic acids, or modulators thereof, can be used to treat pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchioloviveolar carcinoma, bronchial carcinoid, haematoma, and mesenchymal tumors).

As TANGO 261 exhibits expression in the spleen, TANGO 261 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 261 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 261 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 261 exhibits expression in the heart, TANGO 261 nucleic acids, proteins, and modulators thereof can be used to treat heart disorders as described herein.

As TANGO 261 exhibits expression in bone structures, TANGO 261 nucleic acids, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

In another example, TANGO 261 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain. Other examples of such brain and CNS related disorders include but are not limited to bacterial and viral meningitis, Alzheimers Disease, cerebral toxoplasmosis, Parkinson's disease, multiple sclerosis, brain cancers (e.g., metastatic carcinoma of the brain, glioblastoma, lymphoma, astrocytoma, acoustic neuroma), hydrocephalus, and encephalitis.

In another example, TANGO 261 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 261 polypeptides, nucleic acids, or modulators thereof, can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, as TANGO 261 exhibits expression in the brain, TANGO 261 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain. Other examples of such brain and CNS related disorders include, but are not limited to, bacterial and viral meningitis, Alzheimers Disease, cerebral toxoplasmosis, Parkinson's disease, multiple sclerosis, brain cancers (e.g., metastatic carcinoma of the brain, glioblastoma, lymphoma, astrocytoma, acoustic neuroma), hydrocephalus, and encephalitis.

In another example, TANGO 261 polypeptides, nucleic acids, or modulators thereof, can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

In another example, TANGO 261 polypeptides, nucleic acids, or modulators thereof, can be used to treat eye disorders, e.g., retinitis pigmentosa, cataract, retinalastoma, color blindness, conjunctivitis, myopia, dry eyes, keratoconus, glaucoma, macular degeneration, microphthalmia and anophthalmia, nystagmus, and trachoma.

### TANGO 262

In another aspect, the present invention is based on the discovery of nucleic acid sequences which encode a novel family of proteins referred to herein as TANGO 262 proteins. Described herein are human TANGO 262, and mouse TANGO 262 nucleic acid molecules and the corresponding polypeptides which the nucleic acid molecules encode.

Also included within the scope of the present invention are TANGO 262 proteins having a signal sequence.

In certain embodiments, a TANGO 262 family member has the amino acid sequence, and the signal sequence is located at amino acids 1 to 19, 1 to 20, 1 to 21, 1 to 22 or 1 to 23. In such embodiments of the invention, the domains and the mature protein resulting from cleavage of such signal peptides are also included herein. For example, the cleavage of a signal sequence consisting of amino acids 1 to 21 results in a mature TANGO 262 protein corresponding to amino acids 22 to 226 of SEQ ID NO:60. The signal sequence is normally cleaved during processing of the mature protein.

In one embodiment, a TANGO 262 protein includes a signal sequence and is secreted.

### Human TANGO 262

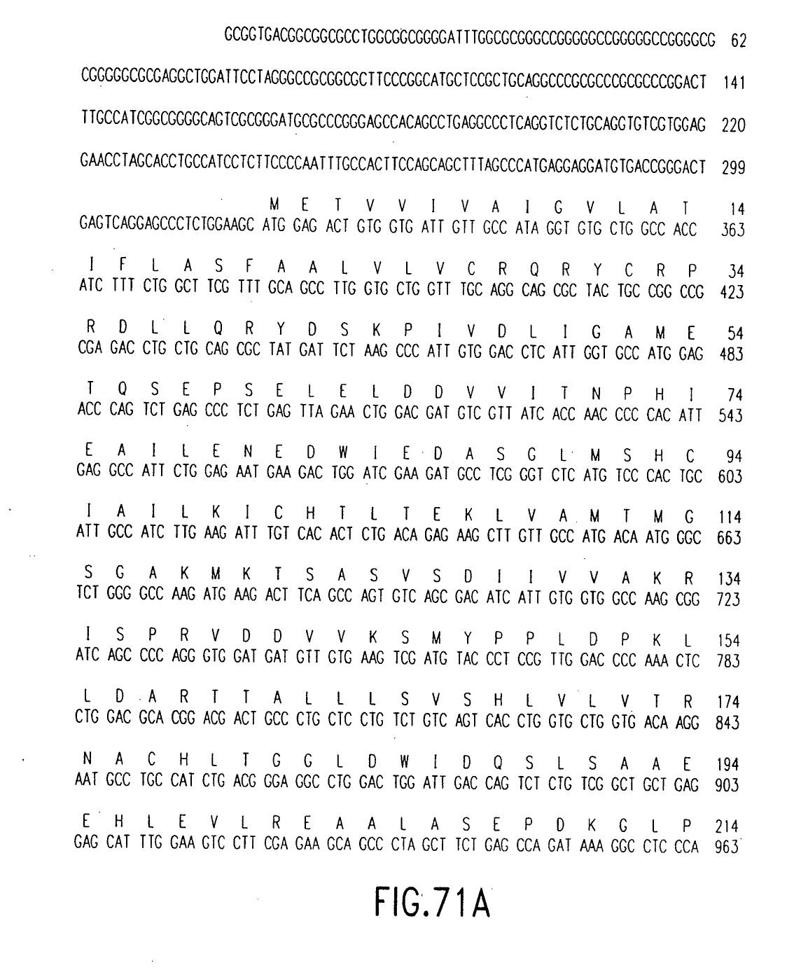

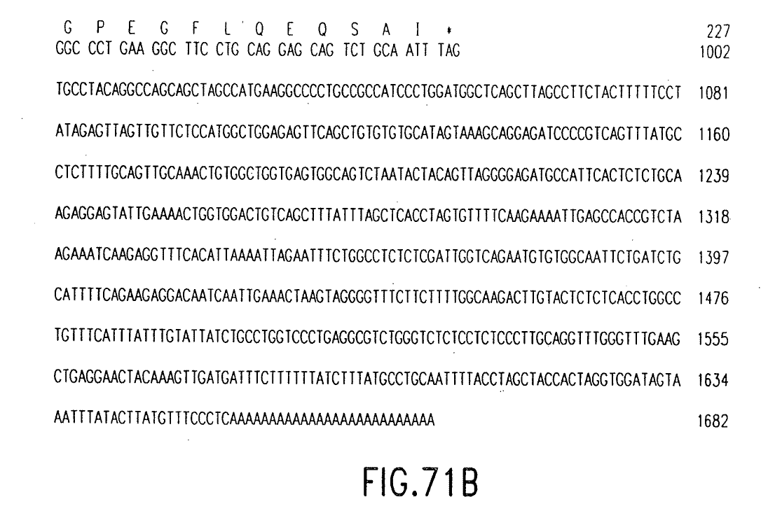

Two clones were originally found in a fetal lung and kidney cell library, as ESTs with similarity to a C. elegans protein encoding gene. The full length sequence was eventually found in a stimulated kidney cell library. A cDNA clone, jthKa045g11, encoding full length human TANGO 262 was identified by screening a stimulated human kidney cell library by EST analysis. The 1682 nucleotide human TANGO 262 sequence (FIG. 71A-71B; SEQ ID NO:59) includes an open reading frame which extends from nucleotide 322 to nucleotide 999 of SEQ ID NO:59 and encodes a 226 amino acid secreted protein depicted in SEQ ID NO:60.

In another embodiment, a cDNA encoding human TANGO 262 was identified by analyzing the sequences of clones present in a human a fetal lung library by EST analysis for sequences that encode wholly secreted or transmembrane proteins. This analysis led to the identification of a clone, jthKa045 g11, comprising a 1510 nucleotide cDNA. The open reading frame of this cDNA comprises nucleotides 325 to 1005, and encodes a transmembrane protein comprising a 226 amino acid polypeptide.

In one embodiment of a nucleotide sequence of human TANGO 262 the nucleotide at position 28 is a guanine (G). In this embodiment, the amino acid at position 2 is glutamate (E). In another embodiment of a nucleotide sequence of human TANGO 262, the nucleotide at position 28 is a cytosine (C). In this embodiment, the amino acid at position 2 is aspartate (D). In another embodiment of a nucleotide sequence of human TANGO 262, the nucleotide at position 483 is a guanine (G). In this embodiment, the amino acid at position 54 is a glutamate (E). In another embodiment of a nucleotide sequence of human TANGO 262, the nucleotide at position 483 is a cytosine (C). In this embodiment, the amino acid at position 54 is aspartate (D). In another embodiment of a nucleotide sequence of human TANGO 262, the nucleotide at position 495 is a guanine (G). In this embodiment, the amino acid at position 58 is a glutamate (E). In another embodiment of a nucleotide sequence of human TANGO 262, the nucleotide at position 495 is a cytosine (C). In this embodiment, the amino acid at position 58 is aspartate (D).

Another embodiment of the invention includes isolated nucleic acid molecules comprising a polynucleotide having a nucleotide sequence encoding the polypeptide having the human TANGO 262 amino acid sequence, but lacking the N-terminal methionine residue. In this embodiment, the nucleotide sequence of human TANGO 262, nucleotides 325-999, encodes the human TANGO 262 amino acid sequence comprising amino acids 2-226.

Human TANGO 262 includes an signal sequence (amino acid 1 to about amino acid 21 of SEQ ID NO:60) preceding the mature protein (about amino acid 22 to amino acid 226 of SEQ ID NO:60). Human TANGO 262 protein, including the signal sequence, has a molecular weight of 24.6 kDa prior to post-translational modification. Mature human TANGO 262 protein has a molecular weight of 22.5 kDa after post-translational modification. The presence of a methionine residue at positions 53, 91, 111, 119, and 146 indicate that there can be alternative forms of human TANGO 262 of 174 amino acids, 136 amino acids, 116 amino acids, 108 amino acids, and 81 amino acids of SEQ ID NO:60, respectively.

In one embodiment, mouse TANGO 262 includes an extracellular domain at amino acids 22 to 226 of SEQ ID NO:60.

A clone, EpT262, which encodes human TANGO 262 was deposited with the American Type Culture Collection (ATCC®, 10801 University Boulevard, Manassas, Va. 520110-2209) on Mar. 26, 1999, and assigned Accession Number 207176. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

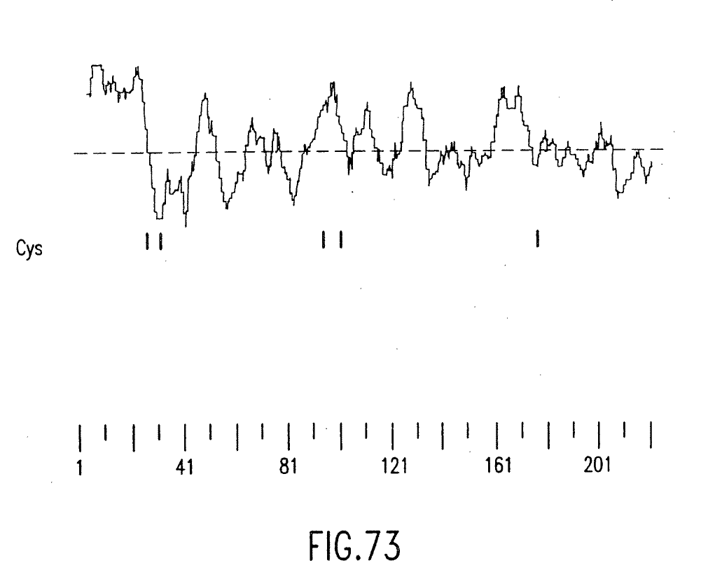

FIG. 73 depicts a hydropathy plot of human TANGO 262. As shown in the hydropathy plot, the hydrophobic region of the plot which corresponds to amino acid 1 to about amino acid 21 is the signal sequence of human TANGO 262.

Northern analysis of human TANGO 262 mRNA expression revealed the presence of an approximately 1.8 kb transcript and an approximately 5.05 kb transcript that are expressed in a range of tissues including strong expression in heart; expression in the brain, skeletal muscle, kidney, liver, small intestine, lung, and placenta. No expression was detected in the colon, thymus, peripheral blood leukocytes, and spleen. The two transcripts likely represent alternative poly A site usage.

Human TANGO 262 is likely expressed in kidney, neuronal cells, placenta, bone, and fetal adrenal tissue, based on the origin of ESTs.

The human gene for TANGO 262 was mapped on radiation hybrid panels to the long arm of chromosome 14, in the region q23-q24. Flanking markers for this region are WI-6253 and WI-5815. The FNTB (fanesyltransferase) and MNAT1 (menage) genes also map to this region of the human chromosome. This region is syntenic to mouse chromosome 12.

### Mouse TANGO 262

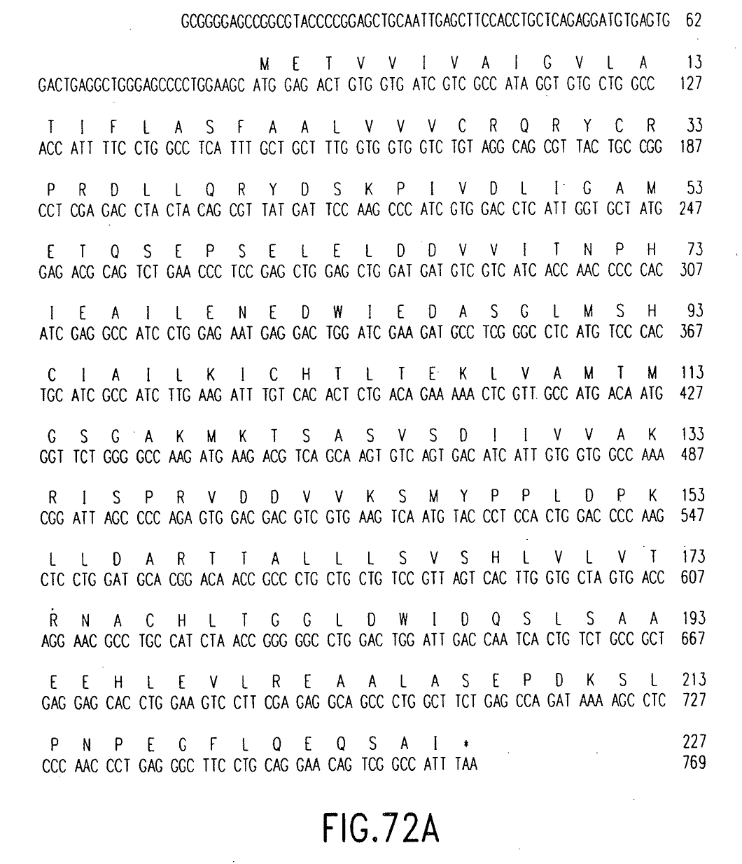

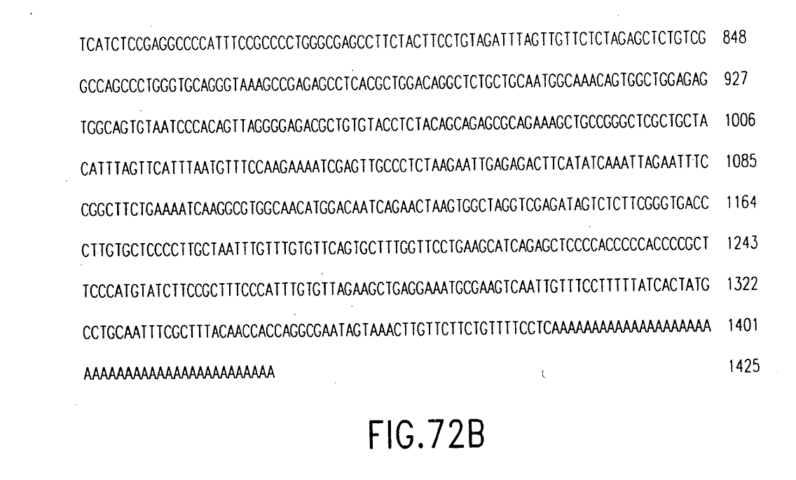

A mouse homolog of human TANGO 262 was identified. A cDNA encoding mouse TANGO 262 was identified by analyzing the sequences of clones present in a mouse microglial cell cDNA library. This analysis led to the identification of a clone, jtmxa002h01, encoding mouse TANGO 262. The mouse TANGO 262 cDNA of this clone is 1425 nucleotides long (FIG. 72A-72B; SEQ ID NO:61). The open reading frame of this cDNA comprises nucleotides 89 to 766 of SEQ ID NO:61, and encodes the 226 amino acid mouse TANGO 262 secreted protein depicted in SEQ ID NO:62.

In another embodiment, a mouse TANGO 262 clone includes comprises a 460 nucleotide cDNA. The open reading frame of this cDNA comprises nucleotides 83 to 460, and encodes a transmembrane protein comprising a 126 amino acid polypeptide.

The signal peptide prediction program SIGNALP (Nielsen et al. (1997) Protein Engineering 10:1-6) predicted that mouse TANGO 262 includes a 21 amino acid signal peptide (amino acids 1 to about amino acid 21 of SEQ ID NO:62) preceding the mature TANGO 262 protein (corresponding to about amino acid 22 to amino acid 226 of SEQ ID NO:62). Mouse TANGO 262 protein, including the signal sequence, has a molecular weight of 24.7 kDa prior to post-translational modification. Mature mouse TANGO 262 protein has a molecular weight of 22.5 kDa after post-translational modification. The presence of a methionine residue at positions 53, 91, 111, 113, 119, and 147 indicate that there can be alternative forms of mouse TANGO 262 of 174 amino acids, 136 amino acids, 116 amino acids, 114 amino acids, 108 amino acids, and 80 amino acids of SEQ ID NO:62, respectively.

In one embodiment of a nucleotide sequence of mouse TANGO 262 the nucleotide at position 94 is a guanine (G). In this embodiment, the amino acid at position 2 is glutamate (E). In another embodiment of a nucleotide sequence of mouse TANGO 262, the nucleotide at position 94 is a cytosine (C). In this embodiment, the amino acid at position 2 is aspartate (D). In another embodiment of a nucleotide sequence of mouse TANGO 262, the nucleotide at position 250 is a guanine (G). In this embodiment, the amino acid at position 54 is a glutamate (E). In another embodiment of a nucleotide sequence of mouse TANGO 262, the nucleotide at position 250 is a cytosine (C). In this embodiment, the amino acid at position 54 is aspartate (D). In another embodiment of a nucleotide sequence of mouse TANGO 262, the nucleotide at position 262 is an adenine (A). In this embodiment, the amino acid at position 58 is a glutamate (E). In another embodiment of a nucleotide sequence of mouse TANGO 262, the nucleotide at position 262 is a cytosine (C). In this embodiment, the amino acid at position 58 is aspartate (D).

Another embodiment of the invention includes isolated nucleic acid molecules comprising a polynucleotide having a nucleotide sequence encoding the polypeptide having the mouse TANGO 262 amino acid polypeptide, but lacking the N-terminal methionine residue. In this embodiment, the nucleotide sequence of mouse TANGO 262, nucleotides, 92-766 of SEQ ID NO:61, encodes the mouse TANGO 262 amino acid sequence comprising amino acids 2-226 of SEQ ID NO:62.

In situ tissue screening was performed on mouse adult and embryonic tissue to analyze for the expression of mouse TANGO 262 mRNA. Expression was widespread during the earlier embryonic ages examined. Expression in the limb, facial, and gut tissues suggested that skeletal muscle may be the predominant contributor to the signal observed in these areas. Strong expression was also seen in the brain and was localized to the area surrounding the lateral ventricles. Spinal cord and other regions of the brain had a significant decrease or lack of expression. Mid and late stage embryos lacked the broad signal seen at earlier ages and had signal in a more defined pattern. The tissues lung, heart, kidney, eye, mucosal epithelium region of the stomach, and the intestinal tract all exhibited strong expression. The area of the brain in contact with the lateral ventricles remained high in expression until E18.5 and then became localized to the choroid plexus. Adult expression remained high in the gut with the stomach, small intestine, and colon all exhibiting strong expression. Kidney and adrenal gland also had expression, as did the choroid plexus as observed in the late stage embryos.

In the case of adult expression, the following results were obtained: A signal was observed in the brain in the choroid plexus of the lateral and 4th ventricles. A strong signal was observed in the mucosal epithelium of the stomach and the colon. A signal was observed in the region of the pericardium of the heart. A weak signal was observed in the ganglion layer of the eye and the harderian gland. A strong, ubiquitous signal was observed in the submandibular gland. A signal was observed in the cortical region of the kidney consistent with the pattern of glomeruli. There was also a ubiquitous signal in the medulla. A strong signal was observed in the cortical region of the adrenal gland. A strong signal was also obtained in the epithelium and villi of the small intestine. A signal was observed in the skeletal muscle/smooth muscle (particularly the diaphragm and peritoneum). A signal was observed in the mucosal epithelium and the serosa of the bladder. No expression was observed in the spinal cord, white fat, brown fat, lung, liver, thymus, lymph node, spleen, and pancreas.

In the case of embryonic expression, the following results were obtained: At E13.5, a signal was observed in a large number of tissues. The signal in the brain was very strong adjacent to the ventricles. The facial region, diaphragm, lung, kidney, and limbs exhibited a very strong signal. A broad expression signal pattern in the limbs suggested developing skeletal muscle. At E14.5, the signal was widely distributed throughout. Tissues lacking strong signal included the brain, except in the regions adjacent to ventricle, the spinal cord, and the liver. At E15.5, a strong signal was observed in the eye, lung, gut, kidney, and the digits of limbs. A signal was also seen in the whisker pads, brain adjacent to the ventricles, Meckel's cartilage, submaxillary gland, heart, and the peritoneum. At E16.5, the signal in the limbs and facial area had decreased to almost background levels suggesting a decrease or loss in signal from developing skeletal muscle. A strong signal was still observed in the eye, ventricle areas of the brain, whisker pads, Meckel's cartilage, submaxillary gland, heart, lung, and kidney. Signal was clearly observed in the mucosal portion of the stomach and the small intestine. At E18.5, the signal pattern is very similar to that observed at E16.5 with the noticeable exception being a significant decrease in signal in the brain adjacent to the ventricles and an increase in signal in the cortical and olfactory bulb areas. The continued decrease in possible muscle or connective tissue signal made the signal in the gut, small intestine and stomach, kidney, lung, and submaxillary gland even more pronounced. At P1.5, a strong signal was observed in the eye, submaxillary gland, kidney, the portion of the stomach containing the mucosal epithelium, and the intestinal tract. A less intense signal was seen in the upper and lower mandible, and the lung. The signal in the brain had decreased to almost background levels except in the choroid plexus.

Human and mouse TANGO 262 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 98.7% over the length of the mouse TANGO 226 protein. The human and mouse TANGO 262 full length cDNAs are 77.0% identical, as assessed using the same software and parameters as indicated (without the BLOSUM 62 scoring matrix). In the respective ORFs, calculated in the same fashion as the full length cDNAs, human and mouse TANGO 262 are 88.5% identical.

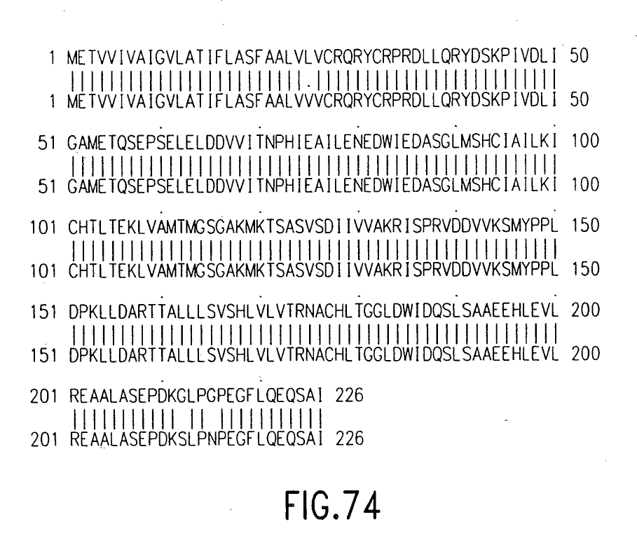

FIG. 74 depicts the alignment of the amino acid sequence of human TANGO 262 and the mouse TANGO 262 amino acid sequence. In this alignment, a (|) between the two sequences indicates an exact match.

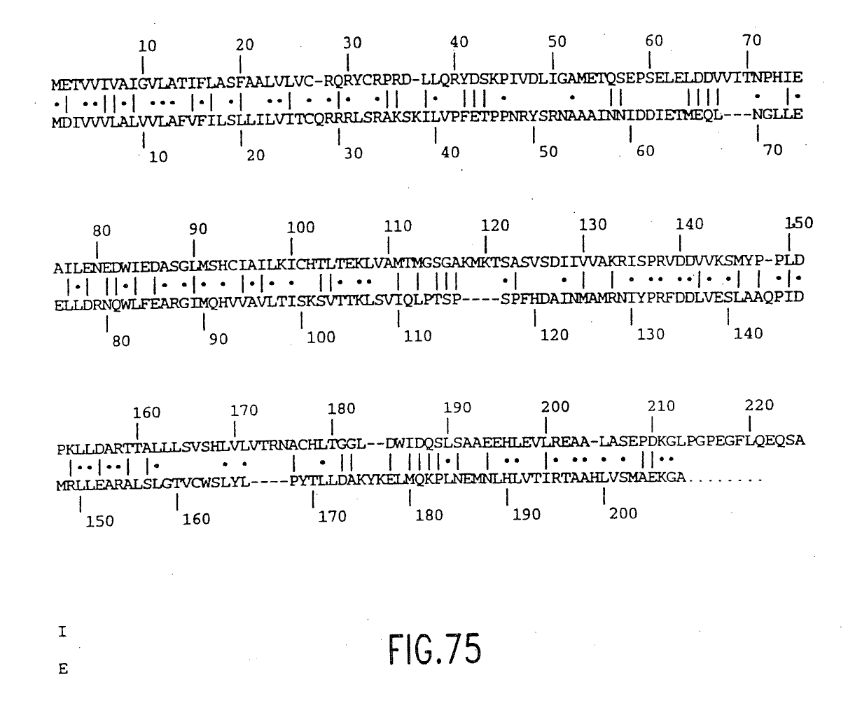

Human TANGO 262 protein bears similarity C elegans protein K10C3.4. Genbank Accession Number AC003687 appears to be the genomic sequence of human TANGO 262 (FIG. 75).

### Uses of TANGO 262 Nucleic Acids, Polypeptides, and Modulators Thereof

The TANGO 262 proteins and nucleic acid molecules of the invention have at least one “TANGO 262 activity” (also referred to herein as “TANGO 262 biological activity”). TANGO 262 activity refers to an activity exerted by a TANGO 262 protein or nucleic acid molecule on a TANGO 262 responsive cell in vivo or in vitro. Such TANGO 262 activities include at least one or more of the following activities: 1) interaction of a TANGO 262 protein with a TANGO 262-target molecule; 2) activation of a TANGO 262 target molecule; 3) modulation of cellular proliferation; 4) modulation of cellular differentiation; or 5) modulation of a signaling pathway. Thus, the TANGO 262 proteins, nucleic acids and/or modulators can be used for the treatment of a disorder characterized by aberrant TANGO 262 expression and/or an aberrant TANGO 262 activity, such as proliferative and/or differentiative disorders.

TANGO 262 proteins, nucleic acids and/or modulators of the invention are useful in the treatment of disorders of the kidney, nervous system, bone, and adrenal gland.

As TANGO 262 is expressed in the kidney, the TANGO 262 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such can be used to treat or modulate renal (kidney) disorders, such as glomerular diseases (e.g. acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As TANGO 262 exhibits expression in the lung, TANGO 262 polypeptides, nucleic acids, or modulators thereof, can be used to treat pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

As TANGO 262 exhibits expression in the heart, TANGO 262 nucleic acids, proteins, and modulators thereof can be used to treat heart disorders as described herein.

As TANGO 262 exhibits expression in the small intestine, TANGO 262 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, TANGO 262 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 262 polypeptides, nucleic acids, or modulators thereof, can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

### TANGO 266

In another aspect, the present invention is based on the discovery of nucleic acid sequences which encode a novel family of proteins referred to herein as TANGO 266 proteins. Described herein is a human TANGO 266 nucleic acid molecule and the corresponding protein which the nucleic acid molecule encodes.

Also included within the scope of the present invention are TANGO 266 proteins having a signal sequence.

In certain embodiments, a TANGO 266 family member has the amino acid sequence, and the signal sequence is located at amino acids 1 to 17, 1 to 18, 1 to 19, 1 to 20 or 1 to 21. In such embodiments of the invention, the domains and the mature protein resulting from cleavage of such signal peptides are also included herein. For example, the cleavage of a signal sequence consisting of amino acids 1 to 19 of SEQ ID NO:64 results in a mature TANGO 266 protein corresponding to amino acids 20 to 105 of SEQ ID NO:64. The signal sequence is normally cleaved during processing of the mature protein.

Thus, in one embodiment, a TANGO 266 protein includes a signal sequence and is secreted.

### Human TANGO 266

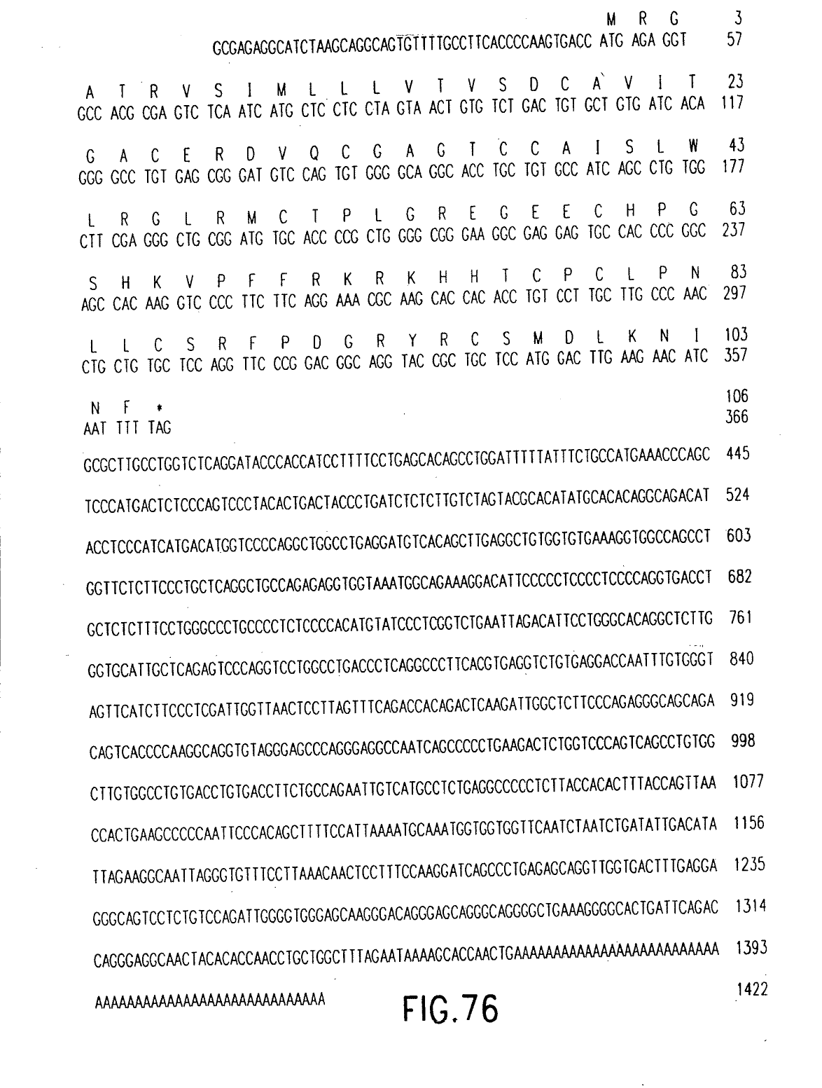

A sequence encoding human TANGO 266 was identified by screening a human adrenal gland library by EST analysis. The 1422 nucleotide human TANGO 266 sequence (FIG. 76; SEQ ID NO:63) includes an open reading frame which extends from nucleotide 49 to nucleotide 363 of SEQ ID NO:63 and encodes a 105 amino acid protein (SEQ ID NO:64).

In another embodiment, a human TANGO 266 clone includes comprises a 422 nucleotide cDNA. The open reading frame of this cDNA comprises nucleotides 56 to 373, and encodes a transmembrane protein comprising an 105 amino acid polypeptide.

In one embodiment of a nucleotide sequence of human TANGO 266 the nucleotide at position 129 is a guanine (G). In this embodiment, the amino acid at position 27 is glutamate (E). In another embodiment of a nucleotide sequence of human TANGO 266, the nucleotide at position 129 is a cytosine (C). In this embodiment, the amino acid at position 27 is aspartate (D). In another embodiment of a nucleotide sequence of human-TANGO 266, the nucleotide at position 216 is an adenine (A). In this embodiment, the amino acid at position 56 is a glutamate (E). In another embodiment of a nucleotide sequence of human TANGO 266, the nucleotide at position 216 is a cytosine (C). In this embodiment, the amino acid at position 56 is aspartate (D). In another embodiment of a nucleotide sequence of human TANGO 266, the nucleotide at position 222 is a guanine (G). In this embodiment, the amino acid at position 58 is a glutamate (E). In another embodiment of a nucleotide sequence of human TANGO 266, the nucleotide at position 222 is a cytosine (C). In this embodiment, the amino acid at position 58 is aspartate (D).

Another embodiment of the invention includes isolated nucleic acid molecules comprising a polynucleotide having a nucleotide sequence encoding the polypeptide having the human TANGO 266 polypeptide, but lacking the N-terminal methionine residue. In this embodiment, the nucleotide sequence of human TANGO 266, nucleotides 52-363 of SEQ ID NO:63, encodes the human TANGO 216 amino acid sequence comprising amino acids 2-105 of SEQ ID NO:64.

Human TANGO 266 includes a signal sequence (amino acid 1 to about amino acid 19 of SEQ ID NO:64) preceding the mature protein (about amino acid 20 to amino acid 105 of SEQ ID NO:64). Human TANGO 266 protein, including the signal sequence, has a molecular weight of 11.7 kDa prior to post-translational modification. Mature human TANGO 266 protein has a molecular weight of 9.7 kDa after post-translational modification. The presence of a methionine residue at positions 10, 49, and 98 indicate that there can be alternative forms of human TANGO 266 of 96 amino acids, 57 amino acids, and 8 amino acids of SEQ ID NO:64, respectively.

A clone, EpT266, which encodes human TANGO 266 was deposited with the American Type Culture Collection (ATCCO, 10801 University Boulevard, Manassas, Va. 20110-2209) on Mar. 26, 1999, and assigned Accession Number 207176. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

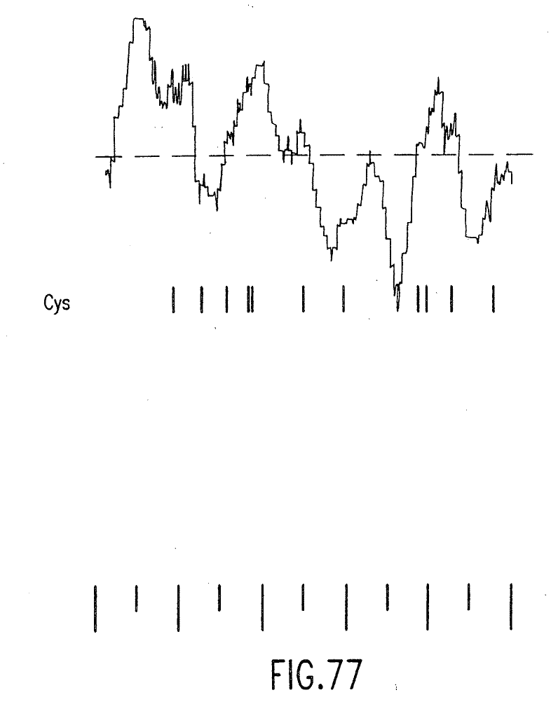

FIG. 77 depicts a hydropathy plot of human TANGO 266. As shown in the hydropathy plot, the hydrophobic region of the plot which corresponds to amino acid 1 to about amino acid 19 is the signal sequence of human TANGO 266.

Northern analysis of human TANGO 266 mRNA expression revealed the presence of an approximately 1.7 kb transcript that is expressed in a range of tissues including very strong expression in placenta; and weak expression in heart. An additional Northern was performed on human TANGO 266 in which strong expression was detected in the adrenal medulla and testis, and moderate expression was detected in the adrenal cortex. No expression was detected in the brain, lung, liver, skeletal muscle, kidney, and pancreas.

In situ tissue screening was performed on mouse adult and embryonic tissue to analyze for the expression of human TANGO 266 mRNA. Consistent with the Northern results obtained above, expression was seen in the ovarian stroma and placenta. The pattern of the signal suggested expression by a component of the vasculature. A stronger signal was observed in the testes. The pattern was multifocal and did not suggest expression by seminiferous tubules. Photoemulsion can be used to determine the exact cellular component of these tissues expressing human TANGO 266 mRNA.

Specifically, in the case of adult expression, a strong, multifocal signal was detected in the testes. A moderate signal was detected in the placenta. No expression was detected in the following tissues: brain (cerebellum), submandibular gland, heart, liver, kidney, colon, small intestine, and spleen.

### Example 1

### Isolation And Characterization of HUMAN TANGO 266 cDNAs

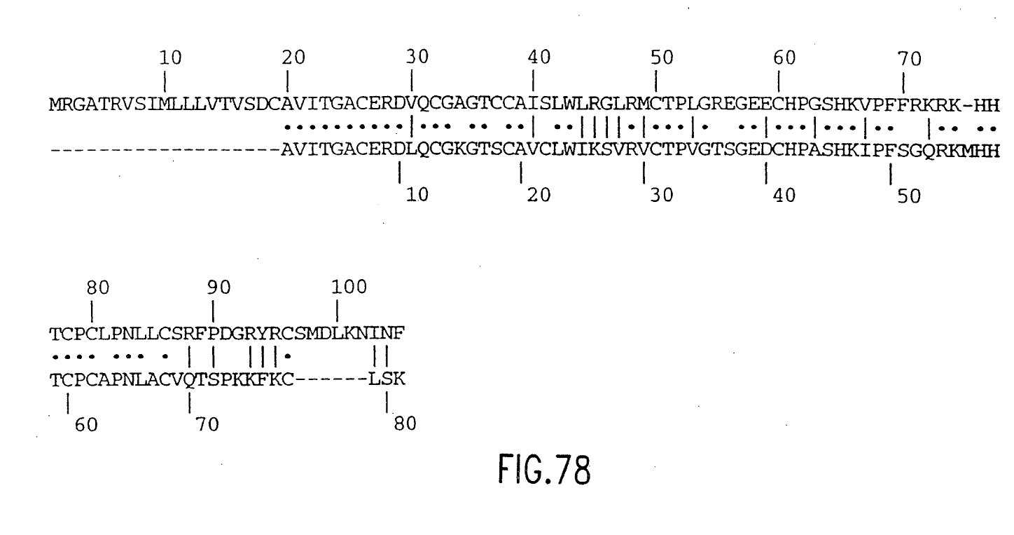

A human TANGO 266 cDNA was isolated from a human adrenal gland cDNA library. A cDNA library from human adult adrenal gland RNA was constructed and sequenced by automated high throughput single pass sequencing, and individual clones analyzed for homology to known proteins. A cDNA clone (TANGO 266) was found initially to have significant homology only to venom protein A (VPRA), found in high abundance in the venom of the black mamba (Dendroaspis polylepsis)(Schweitz, H., Didard, J. & Lazdunski, M. (1990) Toxicon 28, 847-856)(Boisbouvier, J. et al. (1998) J. Mol. Biol. 283, 205-219). TANGO 266 was found to be 58% identical to VPRA over the 81 residues of reported amino acid sequence (FIG. 78). Recently, a similar protein (Bv8) was isolated from skin secretions of the frog Bombina Variegata (Mollay, C. et al. (1999) Eur. J. Pharmacol. 374, 189-196) and the peptide sequence was used to clone the frog, mouse and human Bv8 cDNAs (Wechselberger, C. et al. (1999) FEBS Lett. 462, 177-181). The partial human Bv8 sequence reported was compared to that of TANGO 266 and found have 45% identity over the length of the published sequence.

Human TANGO 266 protein bears similarity to Dendroaspis polypepis polypepis venom protein A (SwissProt Accession Number P25687; Joubert and Strydom (1980) Hoppe Seylers ZPhysiol. Chem. 361:1787-94). FIG. 78 depicts the alignment of the amino acid sequence of human TANGO 266 and Dendroaspis polypepis polypepis venom protein A. In this alignment, a (−) between the two sequences indicates an exact match. The cysteines at residues at positions 26, 32, 38, 50, 60, 78, 80, 86, and 96 of human TANGO 266 (SEQ ID NO:64) are conserved between human TANGO 266 and Dendroaspis polypepis polypepis venom protein A, suggesting that these cysteines form disulfide bonds. A cysteine at amino acid position 37 in TANGO 266 (SEQ ID NO:64) is not found at the corresponding position in Dendroaspis polypepis polypepis venom protein A. However, a tenth cysteine occurs four residues beyond the corresponding position. This tenth cysteine residue is likely able to interact with its partner from either position.

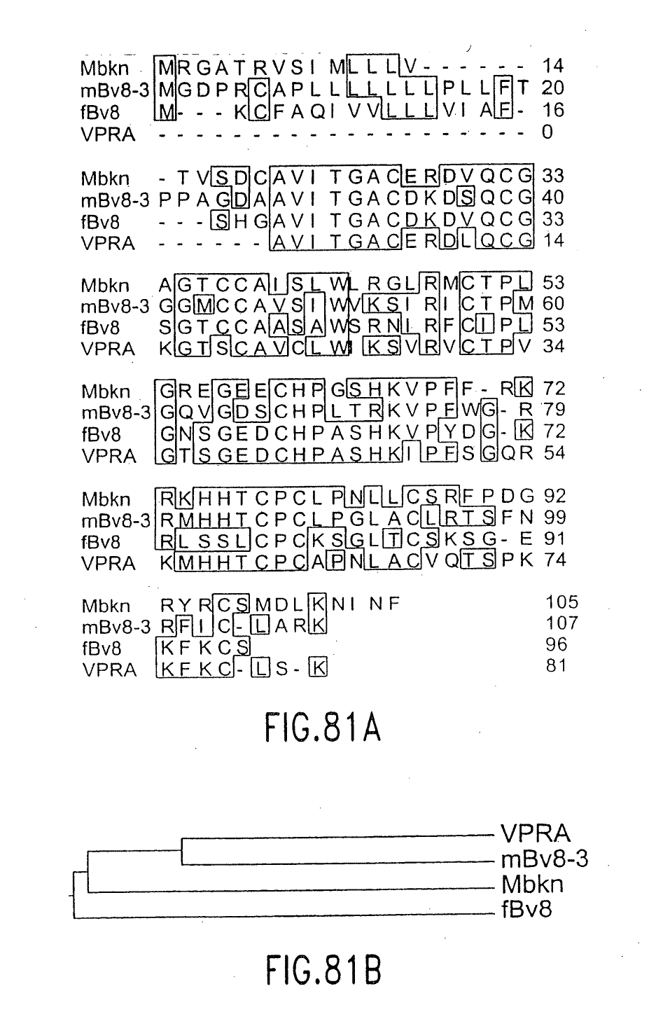

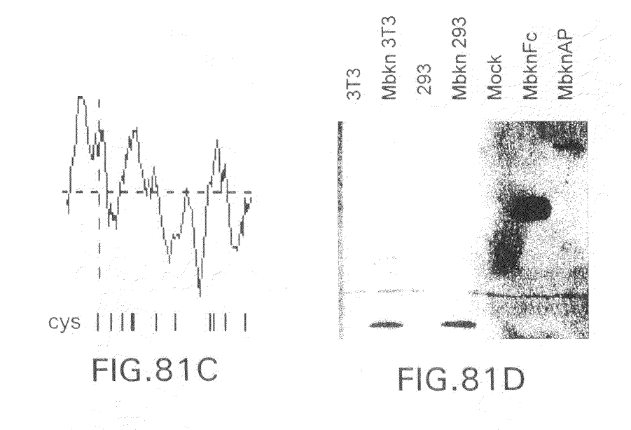

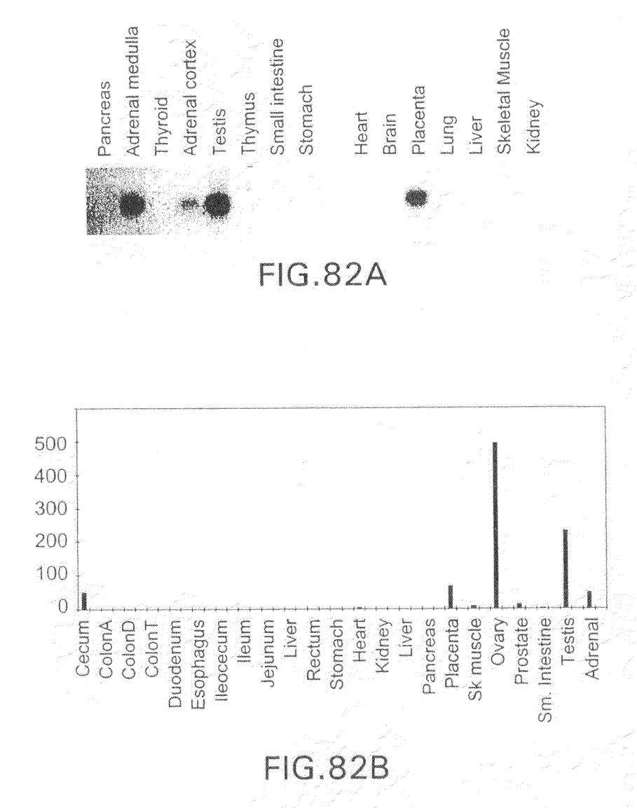

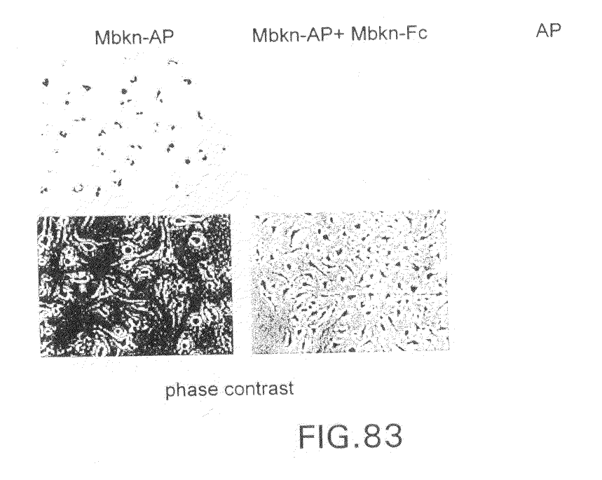

Comparison of mouse Bv8 variant 3 to VPRA and TANGO 266 is shown in FIG. 81A-81D. Mouse Bv8 is closer in homology to VPRA than TANGO 266, with 60% identity over the region of the VPRA peptide sequence, whereas TANGO 266 shares 54% identity with VPRA. The primary structure of TANGO 266 is similar to By8 and VPRA, with identical amino terminal sequences (AVITGAC) and conservation of 10 cysteines in the mature protein, with the exception of VPRA, which lacks the first cysteine. The complete TANGO 266 cDNA (1,422 bp) encodes a 105 residue protein with a predicted molecular mass of 11,714 Daltons.

### Example 2

### Determination of TANGO 266 as Secreted Protein

To determine if the signal peptide prediction correctly determined that TANGO 266 is a secreted protein, cell lines were transfected with TANGO 266 cDNA and subjected to a secretion assay, and their supernatants were probed with rabbit anti human TANGO 266 peptide polyclonal antisera (as discussed below). 293 cells were transfected with expression vectors carrying TANGO 266 Fc-tagged fusion protein, alkaline phosphatase (AP) tagged fusion protein, or with a retroviral vector expressing the native protein.

Media from transfected cells was collected and evaluated by Western for presence of secreted protein (FIG. 81D). In all instances polyclonal anti-TANGO 266 recognized native or tagged protein. In addition, TANGO 266 could be detected in media of 3T3 cells infected with a retrovirus expressing native TANGO 266, but not in control cells infected with an empty vector. The procedures utilized for creation of fusion proteins, for production of the anti-TANGO 266 antibody, and for testing protein secretion, are as follows:

Creation of TANGO 266 Fusion Proteins

TANGO 266 was amplified by PCR and cloned into expression vectors containing different epitope tags. The following oligos were used:

P1: 5′TTTTTGAATTCACCGCCATGAGAGGTGCCACGCGAG 3′
P2: 5′TTTTTCTCGAGAAAATTGATGTTCTTCAAGTCCA 3′
P3: 5′ TTTTTAGATCTGCTGTGATCACAGGGGCC 3′
P4: 5′ TTTTTCTCGAGCTAAAAATTGATGTTCTTCAAGTC 3′

TANGO 266 was amplified with P1 (contains EcoRI site and Kozak sequence) and P2 (contains XhoI site) and cloned in frame into the EcoRI and XhoI sites of the pMEAP3 vector 5′ of alkaline phosphatase (TANGO 266-AP). Using the same sites TANGO 266 was also cloned into pcDNA3.1 containing either the sequence encoding for the Fc part of hIgG1 or a FLAG epitope adding the Fc (TANGO 266-Fc) or Flag (TANGO 266-Flag) sequence in frame to the 3′ end of TANGO 266. Oligos P3 and P4 were used to clone TANGO 266 (without signal peptide) into the Bgl II and XhoI cloning sites of plasmid APTag3, 3′ of alkaline phosphatase and in frame (AP-TANGO 266).

Production of Anti-TANGO 266 Antibody

Polyclonal anti-TANGO 266 was produced in rabbits using the peptide PLGREGEECHPGSHK. Antibody was peptide affinity purified from 12 week bleeds.

Protein Secretion Assay

The sequenced DNA constructs were transiently transfected into HEK 293T cells in 150 mM plates using Lipofectamine (GIBCO/BRL) according to the manufacturer's protocol. 72 hours post-transfection, the serum-free conditioned media (OptiMEM, Gibco/BRL) were harvested, spun and filtered. Alkaline phosphatase activity in conditioned media was quantitated using an enzymatic assay kit (Phospalight) according to the manufacturer's instructions. Conditioned medium samples were analyzed by SDS-PAGE followed by Western blot using polyclonal anti-peptide antibodies to TANGO 266 as described previously.

Isolation of the TANGO 266-Fc was performed with a one step purification scheme utilizing the affinity of the human IgG1 Fc domain to Protein A. The conditioned media was passed over a POROS A column (4.6×100 mm, PerSeptive Biosystems); the column was then washed with PBS, pH 7.4 and eluted with 200 mM glycine, pH 3.0. Samples were dialyzed against PBS, pH 7.4 at 4° C. with constant stirring. The buffered exchanged material was then sterile filtered (0.2 micrometers, Millipore) and frozen at −80° C.

### Example 3

### TANGO 266 Tissue Distribution

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of flourescence using β-2 microglobulin as an internal amplicon reference. The integrity of the RNA samples following DNase I treatment was confirmed by agarose gel electrophoresis and ethidium bromide staining. After phenol extraction cDNA was prepared from the sample using the SuperScript™ Choice System following the manufacturer's instructions (GibcoBRL). A negative control of RNA without reverse transcriptase was mock reverse transcribed for each RNA sample.

Expression was measured by TaqMan® quantitative PCR (Perkin Elmer Applied Biosystems) in cDNA prepared from the following normal human tissues: cecum, colon ascending, colon descending, colon transverse, duodenum, esophagus, ileocecum, ileum, jejunum, liver, rectum, stomach, heart, kidney, liver, pancreas, placenta, skeletal muscle, ovary, prostate, small intestine, testis, and adrenal tissue.

Each TANGO 266 gene probe was labeled using FAM (6-carboxyfluorescein), and the β2-microglobulin reference probe was labeled with a different fluorescent dye, VIC (forward and reverse primers, and TaqMan probe, were designed by PrimerExpress software (PE Biosystems) based on the sequence of each gene). The differential labeling of the target gene and internal reference gene thus enabled measurement in the same well. Forward and reverse primers and the probes for both β2-microglobulin and target gene were added to the TaqMan® Universal PCR Master Mix (PE Applied Biosystems). Although the final concentration of primer and probe could vary, each was internally consistent within a given experiment. A typical experiment contained 200 nM of forward and reverse primers plus 100 nM probe for β-2 microglobulin and 600 nM forward and reverse primers plus 200 nM probe for the target gene. TaqMan matrix experiments were carried out on an ABI PRISM 7700 Sequence Detection System (PE Applied Biosystems).

The following method was used to quantitatively calculate gene expression: The threshold cycle (Ct) value was defined as the cycle at which a statistically significant increase in flourescence was detected. A lower Ct value was indicative of a higher mRNA concentration. The Ct value of the kinase gene was normalized by subtracting the Ct value of the β-2 microglobulin gene to obtain a Ct value using the following formula: Δct=Ct kinase—Ct β-2 microglobulin. Expression was then calibrated against a cDNA sample showing a comparatively low level of expression of the kinase gene. The ΔCt value for the calibrator sample was then subtracted from _{Δ}Ct for each tissue sample according to the following formula: _{Δ}Ct=_{Δ}Ct-sample−_{Δ}Ct-calibrator. Relative expression was then calculated using the arithmetic formula given by 2^{-ΔΔCt}.

TANGO 266 gene expression was as follows: No expression was detected in colon ascending, colon descending, colon transverse, duodenum, esophagus, ileocecum, ileum, jejunum, liver, rectum, stomach, kidney, liver, and pancreas. Trace levels of expression were detected in small intestine, which shall serve as the baseline level of expression, relative to which other levels are compared. Skeletal muscle, heart, and prostate reveal levels of expression about five times greater than the level of expression in small intestine.

Cecum, placenta, and adrenal tissue reveal levels of expression about 40-50 times greater than the level of expression in small intestine. Testis revealed a level of expression about 250 times stronger than the level of expression in small intestine, and in ovary the expression was about 500 times stronger than the level of expression in small intestine.

### Example 4

### Screening of Mouse Tissues for TANGO 266 Binding Sites

To identify potential sites of action of TANGO 266, mouse tissues sections were screened for binding sites using TANGO 266 alkaline phosphatase fusion proteins. Alkaline phosphatase was fused in frame either to the N-terminus (AP-TANGO 266) or the C-terminus (TANGO 266-AP) of TANGO 266. Binding of TANGO 266-AP (as well as AP-TANGO 266) to scattered cells in bone marrow and in the red pulp of spleen was detected. Alkaline Phosphatase (AP) by itself was used as control and did not bind to spleen and bone marrow. The morphology of cells bound by TANGO 266 was reminiscent of cells of the monocyte/macrophage lineage and prompted an analysis of the binding of TANGO 266 to isolated bone marrow derived macrophages. TANGO 266-AP, but not AP by itself, bound to macrophages cultured in vitro for 3 days in the presence of M-CSF (macrophage colony stimulating factor). The binding studies were performed as follows. The isolation of bone marrow derived macrophages is also described below:

Binding studies using alkaline phosphatase fusion proteins were done as described in Cheng and Flanagan, Cell 79:157-168. Briefly, 8 μM cyrostat sections were prepared from tissues embedded in OCT and frozen in liquid nitrogen. Sections were thawed, washed once in HBHA (Hank's balanced salt solution supplemented with 20 mM Hepes, pH 7, 0.05% BSA and 0.1% sodium azide) and incubated with alkaline phospatase fusion proteins for one hour in a humidified chamber. Sections were washed 6 times in HBHA, fixed in acetone/paraformaldehyde, washed 3× in HBS (20 mM Hepes, pH 7.5, 150 mM NaCl) and developed using BCIP/NBT substrate solution (100 mM Tris-HCl, pH 9.5, 100 mM NaCl, 5 mM MgCl, 0.17 mg/ml BCIP and 0.33 mg/ml NBT).

Bone marrow derived macrophages were obtained by culturing nucleated bone marrow cells (see the following section) with 50 ng/ml M-CSF on cover slips in 6-well plates. After three days, non-adherent cells were removed and adherent cells on cover slips were fixed in acetone and air-dried.

### Example 5

### Analysis of the Effect of TANGO 266 on Mononuclear Bone Marrow Cells

The results of the binding studies also prompted an analysis of the effect of purified TANGO 266-Fc on mononuclear bone marrow cells. Cells were cultured in the presence of TANGO 266-Fc for three days and mitogenic activity was measured by ³H thymidine incorporation. TANGO 266-Fc was shown to induce a concentration-dependent increase in the mitogenic response. Maximal ³H thymidine incorporation was detected at about 1500 ng/ml. A control-Fc fusion protein had no effect on the mitogenic response making it unlikely that the Fc part of the protein is responsible for the observed effect. Moreover, heat inactivation of TANGO 266-Fc (10 min at 95 degrees Celsius) abolished the mitogenic response ruling out the possibility that the functional response elicited by TANGO 266-Fc is due to endotoxin contamination in the protein preparation.

Culturing of mononuclear bone marrow cells (described below) in the presence of TANGO 266-Fc not only resulted in a mitogenic response but also in morphological changes. Large numbers of adherent cells of macrophage-like morphology were observed in cultures treated with 266-Fc but only few if any adherent cells were detected in cultures treated with culture medium only, control-Fc or heat-inactivated TANGO 266-Fc. Immuno-fluorescence analysis (discussed briefly below) showed that the adherent cell population was positive for Mac-1, a marker specific for the myeloid lineage and F4/80, a marker specific for macrophages indicating that the adherent cells are macrophages. This was further confirmed by FACS analysis using a range of different lineage markers. The adherent cell population stimulated by TANGO 266-Fc is Mac1+, F4/80−, Gr-1 low, B220- and CD3−. In summary, the above data show that TANGO 266-Fc stimulates a mitogenic response in mononuclear bone marrow cells, and the proliferation and differentiation of macrophages.

Culturing Bone Marrow Cells

Bone marrow was harvested from femurs of 4 to 6 week old C57BL6 mice and passed over a mouse density centrifugation medium (LympolyteM, Cedarlane laboratories, Ontario) to isolate nucleated cells. For the 3H thymidine incorporation assay, 0.5 to 1×10⁵ nucleated cells were incubated in a total volume of 0.2 ml in individual of 96-well plates containing dilutions of TANGO 266 for 72 h. The culture medium used was McCoy's 5A mdium supplemented with 15% fetal calf serum and antibiotics. During the last 6 hours of culture, cells were pulse labeled with 0.5 μCi 3H thymidine (5 Ci/mmol sp. act.) and 3H thymidine incorporation was quantified by scintillation counting as described.

Flow Cytometry and Immuno-fluorescence

For flow cytometry analysis cultures were set up in 6-well plates. Adherent cells were detached in Versene, washed and then incubated for 60 min with 10 μg/ml of the FITC-conjugated marker antibodies. Cells were then washed and analyzed with a FACSCaliber flow cytometer. For in situ fluorescence analysis adherent cells grown on chamberslides were fixed in acetone, washed in PBS and incubated for 60 minutes with FITC-conjugated marker antibodies in a humidified staining chamber. Slides were washed in PBS, mounted with cover slips and analyzed under a fluorescence microscope.

### Example 6

### In Vivo TANGO 266 Expression

To study the consequences of TANGO 266 expression in vivo (described below), we overexpressed TANGO 266 in the hematopoietic system of mice. To this end, hematopoietic progenitor cells from SJL mice were transduced with a retroviral vector carrying TANGO 266 (MSP-TANGO 266) or an empty control vector pMSCVpac (MSP). Transduced cells were then transplanted into sublethally irradiated C57B16 mice and allowed to reconstitute the hematopietic system. Two months after transplant, animals were sacrificed. Blood, bone marrow and spleen were analyzed by flow cytometry with different hematopoietic lineage markers including B220, IgD, CD3, NK1.1, Mac1, Gr-1 and F4/80. CD45.1, a marker specific for donor derived cells, was used as an indicator for the reconstitution efficiency.

The reconstitution efficiency was similar for all animals (about 90%/0). No differences in the distribution of the hematopoietic lineages were seen in blood and borie marrow between mice reconstituted with MSP-TANGO 266 taansduced bone marrow (MSP-TANGO 266 mice) versus mice reconstituted with MSP transduced bone marrow (MSP mice). However, whereas the distribution of B220+, CD3+, NK1.1+ and Gr-1 positive cells was similar in the spleen of MSP-TANGO 266 mice and MSP mice, a higher percentage of Mac 1/F4/80 double positive cells was observed in the spleen of MSP-TANGO 266 mice. This Mac1/F4/80 double positive population was hardly detectable in MSP control animals but was clearly visible in MSP-TANGO 266 animals. Mac1 expression was higher on this population compared to the F4/80 negative population. These results indicate that overexpression of TANGO 266 in the hematopoietic system in mice results in an increase of macrophages in the spleen.

In Vivo Animal Studies

The full length human TANGO 266 cDNA was cloned into pMSCVpac (MSP), a virus containing a PGK promoter driven the puromycin resistance gene. Control virus was the empty virus. The viruses were produced in the 293-EBNA cells by transfecting the retroviral plasmid with two PN8e vectors, one containing the gag/pol construct, PN8e gagpol, from the mouse moloney leukemia virus (MMLV) and the other the VSV-G envelop, PN8e VSV-G. Viral supernatants were collected 48 hours, 72 hours and 96 hours after transfection, filtered and centrifuged at 4 C at 50,000×g (25,000 rpm) for 2 hr. Concentrated virus pellets were resuspended in culture medium, shaken and frozen at −80° C. until transduction.

Donor mouse bone marrow cells were collected 4 days after treatment with 5-fluorouracil (5-FU), immunopurified for CD3e, CD11b, CD45R and Ly-6G negative cells, prestimulated for two days, infected for one day with the viral supernatant in the presence of recombinant mouse interleukin-3, recombinant mouse interleukin-6 (nnIL6), recombinant mouse stem cell factor (rmSCF), recombinant mouse fns-like tyrosine kinase-3 ligand (rmFlt-3L) and mouse thrombopoietin (mTPO) and then collected and injected into lethally irradiated recipient mice.

### Example 8

### Analysis of Progenitor Cells to Determine TANGO 266 Effect

In recent years culture conditions have been developed that allow human bone marrow CD34+ progenitors to expand in vitro and to differentiate into antigen presenting cells. (Zandstra, P. W., et al (1997). Proc. Natl. Acad. Sci. USA 94, 4698-4703; Bhafia, M. et al. (1997) J. Exp. Med. 186, 619-624; and Banchereau, J., & Steinman, R. M. (1998) Nature 392, 245-252.) CD34+ human bone marrow cells were cultured in serun free media in the presence of Flt-3 ligand, SCF, IL-3 and IL-6 in the presence or absence of TANGO 5266-Fc. Under these conditions, total cell numbers in cytokines alone or with a control Fc fusion protein increased 200-400 fold. TANGO 266-Fc increased the proportion of adherent cells in expanded human bone marrow CD34+ cell cultures in a dose dependent manner. The morphology of the adherent cells was suggestive of cells differentiating into the monocyte/macrophage lineage.

Cells were assessed for stage of differentiation using CD34, an early hematopoietic progenitor marker, and CD14 and CD1.6 which are expressed by cells that have differentiated into the monocyte/macrophage lineage. CD14 is a functional receptor on cells of the monocytic lineage for bacterial lipopolysaccharide, and for clearance and phagocytosis of apoptotic cells. The addition of TANGO 266-Fc increased the number of cells expressing CD 16. The addition of TANGO 266-Fc greatly decreased the percentage of CD34+/CD14− cells, and increased CD34−/CD14+ cells after 14 days of culture, suggesting that TANGO 266 acts on early progenitors to induce differentiation into the monocyte lineage. This affect was not evident in media alone, with a control Fc fusion protein, or with heat inactivated TANGO 266-Fc. Total cell number after 2 weeks in culture increased 1.5-2.2 fold compared to media alone or in presence of a control Fc protein. The total number of CD34+ cells in culture dropped 10 fold, with a concomitant 3 fold increase in the number of CD14+ cells when cultured in the presence of 200 ng ml⁻¹ TANGO 266-Fc compared to a control Fc. This effect was seen in a dose dependent manner in a range of 1-500 ng ml⁻¹ when cultured for a 2 week period. The human bone marrow cell culture and analysis is described as follows:

Human Bone Marrow CD34⁺ Cell Culture and Analysis

Adult human bone marrow cells selected for expression of CD34 were purchased from Purecell (Foster City, Calif.). Cells (4×10 ml⁻¹) were cultured for 14 days in semm free media containing cytokines (StemCell Tech., Vancouver, B.C., Canada) Flt-3 ligand (100 ng ml⁻¹), SCF (100 ng ml⁻¹), IL-3 (10 ng ml⁻¹) and IL-6 (10 ng ml⁻¹) in a humidified 5% CO₂ incubator at 37° C. Non adherent cells were collected and adherent cells removed by with a cell lifter after incubation in Versene (Gibco/BRL, Grand Island, N.Y.), washed and blocked with 1 mg ml⁻¹ human gamma globulin (Gamimune; Miles Inc, Elkhart, Ind.). Total viable cell count was determined by trypan blue exclusion. Fluorescein isothiocyanate (FITC) labeled anti-CD14 and anti-CD16, and phycoerythrin (PE) labeled anti-CD34 were obtained from Pharmingen. After dilution in PBS cells were analyzed by FACSCaliber flow cytometer (Becton Dickinson, Franklin Lakes, N.J.).

### Example 9

### Mapping Results of TANGO 266

The TANGO 266 nucleic acid sequence bears homology to a marker called SHGC-16135, which is known to map to 1p21. 1p21 is a locus for a disorder known as osteopetrosis, autosomal dominant, type II, the mapping of which was discovered during a study of an extended family with type II disorder (Van Hul, W. et al (1997) Medizinische Genetik 9: 8). In the study, linkage between the disorder and to microsatellite markers in the 1p21 region was demonstrated. The chromosomal region was further analyzed, within which was discovered the gene for macrophage colony stimulating factor (CSF 1), a hematopoietic growth factor that plays an important role in the proliferation of macrophages and osteoclasts from hematopoietic stem cells. Refined mapping appeared to exclude CSF1 as the site of the mutation in the subject family.

### Uses of TANGO 266 Nucleic Acids, Polypeptides, and Modulators Thereof

The TANGO 266 proteins and nucleic acid molecules of the invention have at least one “TANGO 266 activity” (also referred to herein as “TANGO 266 biological activity”). TANGO 266 activity refers to an activity exerted by a TANGO 266 protein or nucleic acid molecule on a TANGO 266 responsive cell in vivo or in vitro. Such TANGO 266 activities include at least one or more of the following activities: 1) interaction of a TANGO 266 protein with a TANGO 266-target molecule; 2) activation of a TANGO 266 target molecule; 3) modulation of cellular proliferation; 4) modulation of cellular differentiation; or 5) modulation of a signaling pathway. Thus, the TANGO 266 proteins, nucleic acids and/or modulators can be used for the treatment of a disorder characterized by aberrant TANGO 266 expression and/or an aberrant TANGO 266 activity, such as proliferative and/or differentiative disorders.

As cytokines are often found in snake venom, and due to TANGO 266's significant homology to enom protein A (VPRA), found in high abundance in the venom of the black mamba (see experimental section), TANGO 266 may be a cytokine. In the same fashion as a cytokine, TANGO 266 has been shown to play a role in the proliferation and differentiation of cells, e.g., macrophages and monocytes, and can therefore be used to treat proliferative and cell differentiation-related disorders. Such proliferative disorders include but are not limited to e.g., carcinoma, e.g., lymphoma, e.g., follicular lymphoma.

Due to its ability to induce the proliferation and differentiation of white blood cell types, e.g., macrophages and monocytes, TANGO 266 polypeptides, nucleic acids, and/or modulators thereof, can be used to treat can be used to treat include immune disorders, e.g., viral disorders (e.g., infection by HSV), cell growth disorders, e.g., cancers (e.g., carcinoma, lymphoma, e.g., follicular lymphoma), autoimmune disorders (e.g., arthritis, graft rejection (e.g., allograft rejection), T cell disorders (e.g., AIDS)) and inflammatory disorders (e.g., bacterial infection, psoriasis, septicemia, cerebral malaria, inflammatory bowel disease, arthritis (e.g., rheumatoid arthritis, osteoarthritis), and allergic inflammatory disorders (e.g., asthma, psoriasis)).

Furthermore, TANGO 266 polypeptides, nucleic acids, and/or modulators thereof, can be used to treat disorders associated with leukocytes, eg., with monocytes, macrophages, lymphocytes, and granulocytes, such as leukopenias (e.g., neutropenia, monocytopenia, lymphopenia, and granulocytopenia), leukocytosis (e.g., granulocytosis, lymphocytosis, eosinophilia, monocytosis, acute and chronic lymphadenitis), malignant lymphomas (e.g., Non-Hodgkin's lymphomas, Hodgkin's lymphomas, leukemias, agnogenic myeloid metaplasia, multiple myeloma, plasmacytoma, Waldenstrom's macroglobulinemia, heavy-chain disease, monoclonal gammopathy, histiocytoses, eosinophilic granuloma, and angioimmunoblastic lymphadenopathy).

Due to its ability to induce the proliferation and differentiation of white blood cell types, e.g., macrophages and monocytes, TANGO 266 polypeptides, nucleic acids, and/or modulators thereof, can be used to treat hematopoeitic disorders.

For example, hematopoeitic disorders that TANGO 266 polypeptides, nucleic acids, and/or modulators thereof can be used to treat include disorders associated with abnormal monocyte and/or macrophage function, such as impaired phagocytosis, chemotaxis, or secretion of cytokines, growth factors and acute-phase reactants, resulting from certain diseases, e.g., lysosomal storage diseases (e.g., Gaucher's disease); impaired monocyte cytokine production, for example, found in some patients with disseminated nontuberculous mycobacterial infection who are not infected with HIV; leukocyte adhesion deficiency (LAD), hyperimmunoglobulin E-recurrent infection (HIE) or Job's syndrome, Chédiak-Higashi syndrome (CHS), and chronic granulomatous diseases (CGD), certain autoimmune diseases, such as systemic lupus erythematosus and other autoimmune diseases characterized by tissue deposition of immune complexes, as seen in Sjögren's syndrome, mixed cryoglobulinemia, dermatitis herpetiformis, and chronic progressive multiple sclerosis. Also included are disorders or infections that impair mononuclear phagocyte function, for example, influenza virus infection and AIDS.

Monocyte associated disorders include monocytoses such as, for example, monocytoses associated with certain infections such as tuberculosis, brucellosis, subacute bacterial endocarditis, Rocky Mountain spotted fever, malaria, and visceral leishmaniasis (kala azar), in malignancies, leukemias, myeloproliferative syndromes, hemolytic anemias, chronic idiopathic neutropenias, and granulomatous diseases such as sarcoidosis, regional enteritis, and some collagen vascular diseases.

Other monocyte associated disorders include monocytopenias such as, for example, monocytopenias that can occur with acute infections, with stress, following administration of glucocorticoids, aplastic anemia, hairy cell leukemia, and acute myelogenous leukemia and as a direct result of administration of myelotoxic and immunosuppressive drugs.

As TANGO 266 is expressed in the spleen library, TANGO 266 nucleic acids, proteins, and/or modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. TANGO 266 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus TANGO 266 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 266 is expressed in the heart, TANGO 266 nucleic acids, proteins, and modulators thereof can be used to treat heart disorders as described herein.

As TANGO 266 is expressed in the pituitary, TANGO 266 polypeptides, nucleic acids, and/or modulators thereof, can be used to treat disorders of the pituitary gland. The pituitary secretes such hormones as thyroid stimulating hormone (TSH), follicle stimulating hormone (FSH), adrenocotropic hormone (ACTH), and others. It controls the activity of many other endocrine glands (thyroid, ovaries, adrenal, etc.). For example, such molecules can be used to treat or modulate pituitary related disorders including, without limitation, acromegaly, Cushing's syndrome, craniopharyngiomas, Empty Sella syndrome, hypogonadism, hypopituitarism, and hypophysitis, in addition to disorders of the endocrine glands the pituitary controls.

As TANGO 266 is expressed in the thyroid, TANGO 266 polypeptides, nucleic acids, and/or modulators thereof, can be used to treat disorders of the thyroid gland, such as hyperthyroidism (e.g., diffuse toxic hyperplasia, toxic multinodular goiter, toxic adenoma, and acute or subacute thyroiditis), hypothyroidism (e.g., cretinism and myxedema), thyroiditis (e.g., Hashimoto's thyroiditis, subacute granulomatous thyroiditis, subacute lymphocytic thyroiditis, Riedel's thyroiditis), Graves' disease, goiter (e.g., simple diffuse goiter and multinodular goiter), or tumors (e.g., adenoma, papillary carcinoma, follicular carcinoma, medullary carcinoma, undifferentiated malignant carcinoma, Hodgkin's disease, and non-Hodgkin's lymphoma).

As TANGO 266 is expressed in adrenal tissue, e.g., in adrenal medulla and adrenal cortex, TANGO 266 polypeptides, nucleic acids, and/or modulators thereof, can be used to treat disorders of the adrenal cortex, such as hypoadrenalism (e.g. primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma). In another example, TANGO 266 polypeptides, nucleic acids, and/or modulators thereof, can be used to treat disorders of the adrenal medulla, such as neoplasms (e.g., pheochromocytomas, neuroblastomas, and ganglioneuromas).

As TANGO 266 is expressed in gonadal tissue, TANGO 266 polypeptides, nucleic acids and/or modulators thereof can be used to modulate the function, morphology, proliferation and/or differentiation of cells in the reproductive tract, particularly in the ovaries and testis.

For example, the TANGO 266 polypeptides, nucleic acids and/or modulators thereof can be used to treat or modulate disorders associated with the testis including, without limitation, the Klinefelter syndrome (both the classic and mosaic forms), XX male syndrome, variococele, germinal cell aplasia (the Sertoli cell-only syndrome), idiopathic azoospermia or severe oligospermia, crpytochidism, and immotile cilia syndrome, or testicular cancer (primary germ cell tumors of the testis). In another example, TANGO 266 polypeptides, nucleic acids, and/or modulators thereof, can be used to treat testicular disorders, such as unilateral testicular enlargment (e.g., nontuberculous, granulomatous orchitis), inflammatory diseases resulting in testicular dysfunction (e.g., gonorrhea and mumps), and tumors (e.g., germ cell tumors, interstitial cell tumors, androblastoma, testicular lymphoma and adenomatoid tumors).

For example, the TANGO 266 polypeptides, nucleic acids and/or modulators thereof can be used modulate the function, morphology, proliferation and/or differentiation of the ovaries. For example, such molecules can be used to treat or modulate disorders associated with the ovaries, including, without limitation, ovarian tumors, McCune-Albright syndrome (polyostotic fibrous dysplasia). In another example, TANGO 266 polypeptides, nucleic acids, and/or modulators thereof, can be used to treat ovarian disorders, such as ovarian endometriosis, non-neoplastic cysts (e.g., follicular and luteal cysts and polycystic ovaries) and tumors (e.g., tumors of surface epithelium, germ cell tumors, ovarian fibroma, sex cord-stromal tumors, and ovarian cancers (e.g., metastatic carcinomas, and ovarian teratoma). For example, the TANGO 266 polypeptides, nucleic acids and/or modulators can be used in the treatment of infertility.

The TANGO 266 polypeptides, nucleic acids and/or modulators thereof can additionally be used to modulate the function, morphology, proliferation and/or differentiation of cells in the tissues of the reproductive tract other than the ovaries and testis. For example, such molecules can be used to treat or modulate disorders associated with the female reproductive tract including, without limitation, uterine disorders, e.g., hyperplasia of the endometrium, uterine cancers (e.g., uterine leiomyomoma, uterine cellular leiomyoma, leiomyosarcoma of the uterus, malignant mixed mullerian Tumor of uterus, uterine Sarcoma), and dysfunctional uterine bleeding (DUB).

As TANGO 266 is expressed in the placenta, TANGO 266 polypeptides, nucleic acids, and/or modulators thereof, can be used to treat placental disorders, such as toxemia of pregnancy (e.g., preeclampsia and eclampsia), placentitis, or spontaneous abortion.

As TANGO 266 maps to the same region as the locus for osteopetrosis, autosomal dominant, type II, and as both macrophages and osteoclasts are derived from the same progenitor cell type, e.g., monocytes, TANGO 266 polypeptides, nucleic acids and/or modulators thereof can be used to modulate the function, morphology, proliferation and/or differentiation of bone and cartilage cells, e.g., osteoclasts, osteoclasts, and chondrocytes. Thus TANGO 266 polypeptides, nucleic acids and/or modulators thereof can be used to treat bone disorders, including but not limited to bone cancer, achondroplasia, osteopetrosis (e.g., osteopetrosis, autosomal domainant, type II), myeloma, fibrous dysplasia, scoliosis, osteoarthritis, osteosarcoma, osteoporosis, and bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

### TANGO 267

In another aspect, the present invention is based on the discovery of nucleic acid sequences which encode a novel family of proteins referred to herein as TANGO 267 proteins. Described herein is a human TANGO 267 nucleic acid molecule and the corresponding protein which the nucleic acid molecule encodes.

An TANGO 267 family member can also include a MAGE-like domain. The MAGE-like domain typically includes about 50 to 250, preferably about 75 to 225, more preferably about 120 to 200, still more preferably about 150 to 180 amino acid residues in length. The MAGE-like cytoplasmic domain typically has the following consensus sequence: [L-Xaa(6)-L-V-Xaa(2)-L-Xaa(2)-K-Xaa(n1)-E-M-L-Xaa(n2)-F-G-Xaa(2)-L-K-E-Xaa-D-Xaa(n3)-G-L-L], wherein L is leucine, Xaa is any amino acid, V is valine, K is lysine, n1 is about 2-15, preferably 5-12, and more preferably 10, E is glutamate, M is methionine, n2 is about 10-40, preferably 15-30, and more preferably 25, F is phenylalanine, G is glycine, D is aspartate, and n3 is 15-40, preferably 20-32, and more preferably 27-28.

### Human TANGO 267

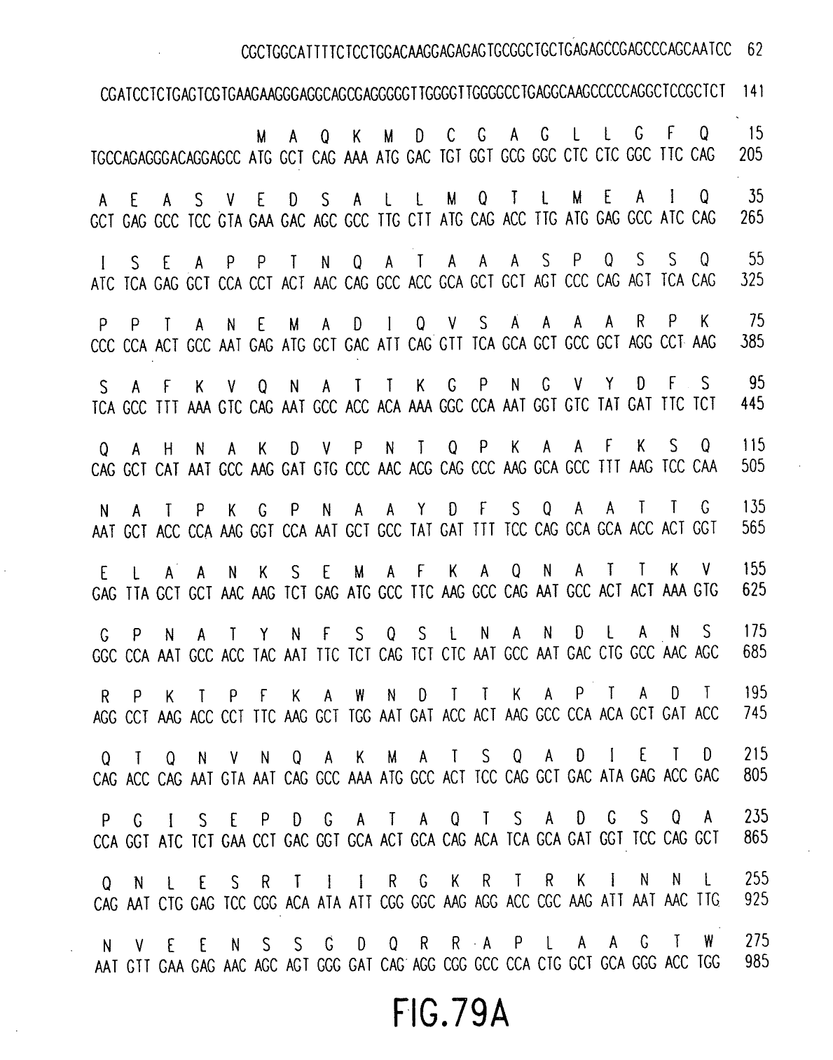

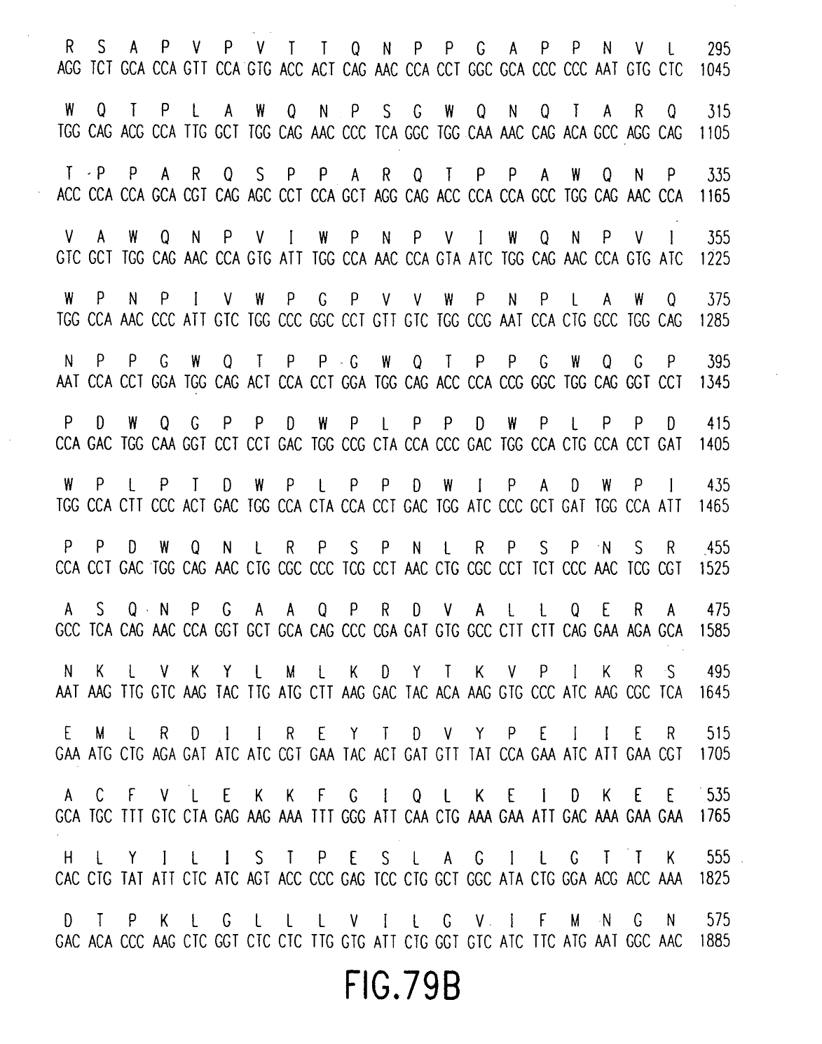

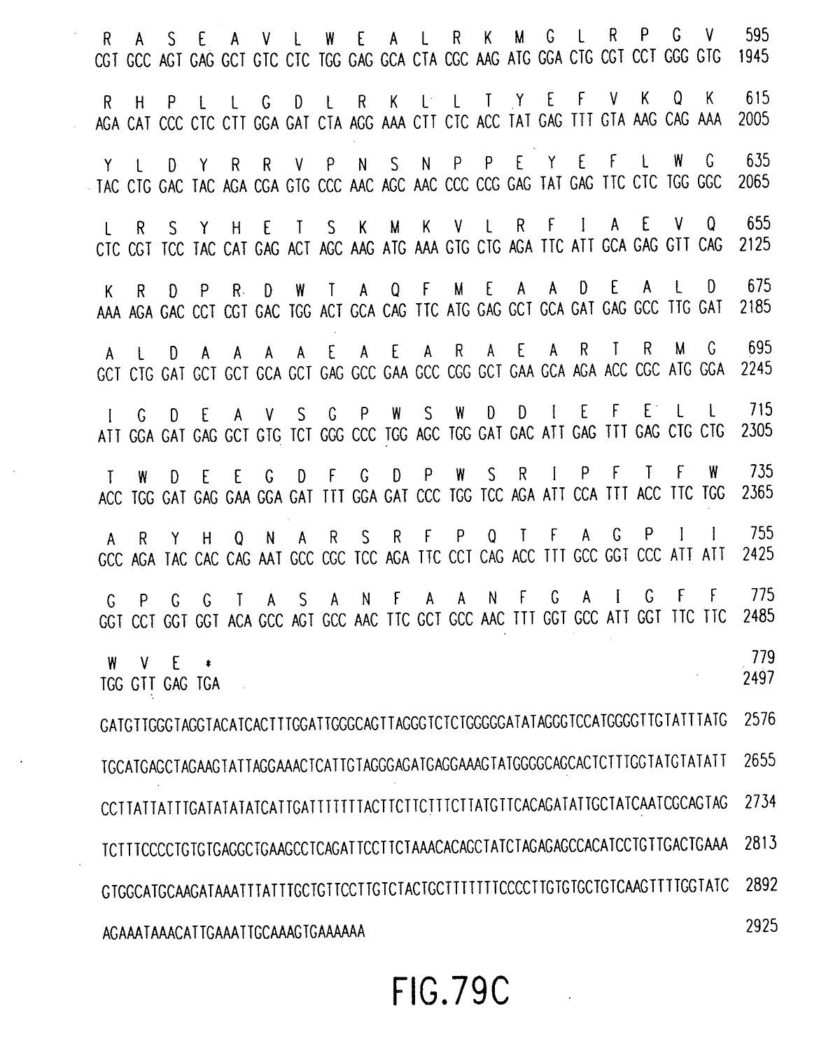

A sequence encoding human TANGO 267 was identified by screening a human coronary artery smooth muscle cell by EST analysis. The 2925 nucleotide human TANGO 267 sequence (FIG. 79A-79C; SEQ ID NO:65) includes an open reading frame which extends from nucleotide 161 to nucleotide 2494 of SEQ ID NO:65 and encodes a 778 amino acid transmembrane protein depicted in SEQ ID NO:66.

In another embodiment, a human TANGO 267 clone includes comprises a 2739 nucleotide cDNA. The open reading frame of this cDNA comprises nucleotides 171 to 2507, and encodes a transmembrane protein comprising a 778 amino acid polypeptide.

In one embodiment of a nucleotide sequence of human TANGO 267 the nucleotide at position 211 is a guanine (G). In this embodiment, the amino acid at position 17 is glutamate (E). In another embodiment of a nucleotide sequence of human TANGO 267, the nucleotide at position 211 is a cytosine (C). In this embodiment, the amino acid at position 17 is aspartate (D). In another embodiment of a nucleotide sequence of human TANGO 267, the nucleotide at position 223 is an adenine (A). In this embodiment, the amino acid at position 21 is a glutamate (E). In another embodiment of a nucleotide sequence of human TANGO 267, the nucleotide at position 223 is a cytosine (C). In this embodiment, the amino acid at position 21 is aspartate (D). In another embodiment of a nucleotide sequence of human TANGO 267, the nucleotide at position 256 is a guanine (G). In this embodiment, the amino acid at position 32 is a glutamate (E). In another embodiment of a nucleotide sequence of human TANGO 267, the nucleotide at position 256 is a cytosine (C). In this embodiment, the amino acid at position 32 is aspartate (D).

Another embodiment of the invention includes isolated nucleic acid molecules comprising a polynucleotide having a nucleotide sequence encoding the polypeptide having the human TANGO 267 amino acid sequence in SEQ ID NO:66, but lacking the N-terminal methionine residue. In this embodiment, human TANGO 267 (nucleotides 164-2494 of SEQ ID NO:65) encodes the human TANGO 267 amino acid sequence from amino acids 2-778 of SEQ ID NO:66.

Human TANGO 267 protein has a molecular weight of 86.2 kD prior to post-translational modification. The presence of a methionine residue at positions 5, 27, 31, 62, 144, 205, 483, 497, 572, 589, 645, 667, and 694 indicate that there can be alternative forms of human TANGO 267 of 774 amino acids, 752 amino acids, 748 amino acids, 717 amino acids, 635 amino acids, 574 amino acids, 296 amino acids, 282 amino acids, 207 amino acids, 190 amino acids, 134 amino acids, 112 amino acids and 83 amino acids of SEQ ID NO:66, respectively.

A clone, EpT267, which encodes human TANGO 267 was deposited with the American Type Culture Collection (ATCC®, 10801 University Boulevard, Manassas, Va. 20110-2209) on Mar. 26, 1999, and assigned Accession Number 207176. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

The present invention also includes TANGO 267 proteins having a transmembrane domain. As used herein, a transmembrane domain refers to an amino acid sequence having at least about 25 to about 40 amino acid residues in length and which contains at least about 65-70% hydrophobic amino acid residues such as alanine, leucine, isoleucine, phenylalanine, pro line, tyrosine, tryptophan, or valine. In a preferred embodiment, a transmembrane domain contains at least about 30-35 amino acid residues, preferably about 30-35 amino acid residues, and has at least about 60-80%, more preferably 65-75%, and more preferably at least about 68% hydrophobic residues. An example of a transmembrane domain includes from about amino acids 559 to 575 of TANGO 267.

In one embodiment, human TANGO 267 includes extracellular domains at amino acids 1 to 558 of SEQ ID NO:66 and amino acids 773 to 778 of SEQ ID NO:66, transmembrane (TM) domains at amino acids 559 to 575 and amino acids 749 to 772 of SEQ ID NO:66; and a cytoplasmic domain at amino acids 576 to 748 of SEQ ID NO:66.

Alternatively, in another embodiment, a human TANGO 267 protein contains an extracellular domain at amino acid residues 576 to 748 of SEQ ID NO:66, transmembrane domains at amino acid residues 147 to 170 and amino acid residues 749 to 772 of SEQ ID NO:66, cytoplasmic domains at amino acid residues 1 to 558 of SEQ ID NO:66 and amino acid residues 743 to 778 of SEQ ID NO:66.

The human gene for TANGO 267 was mapped on radiation hybrid panels to the long arm of chromosome X, in the region q12. Flanking markers for this region are WI-5587 and WI-5717. The AR (androgen receptor), MSN (moesin), and OPHN (oligophrenin 1) genes also map to this region of the human chromosome. This region is syntenic to mouse chromosome X. The gs (greasy) loci also maps to this region of the mouse chromosome. The ar (androgen receptor) and sla (sex linked anemia) genes also map to this region of the mouse chromosome.

Human TANGO 267 appears to be expressed in a wide range of tissues based on EST origin.

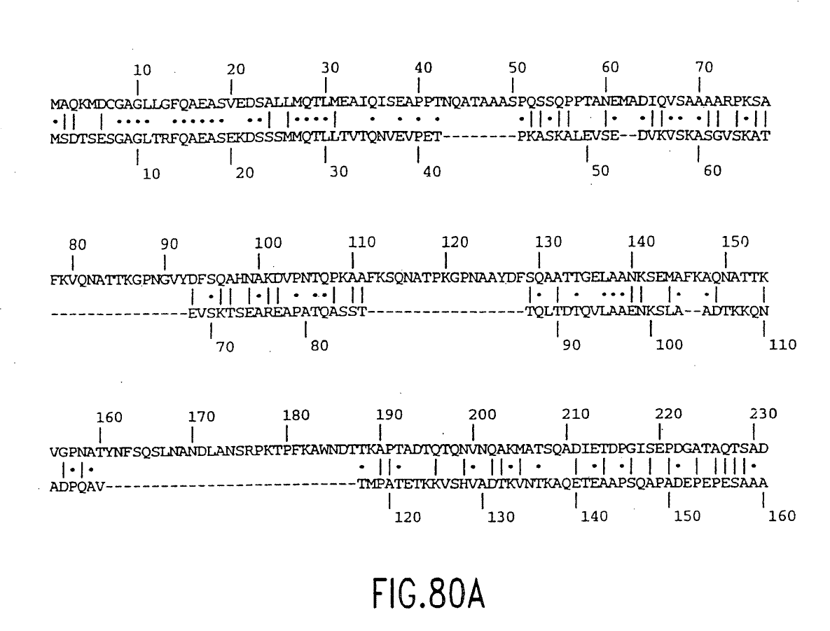

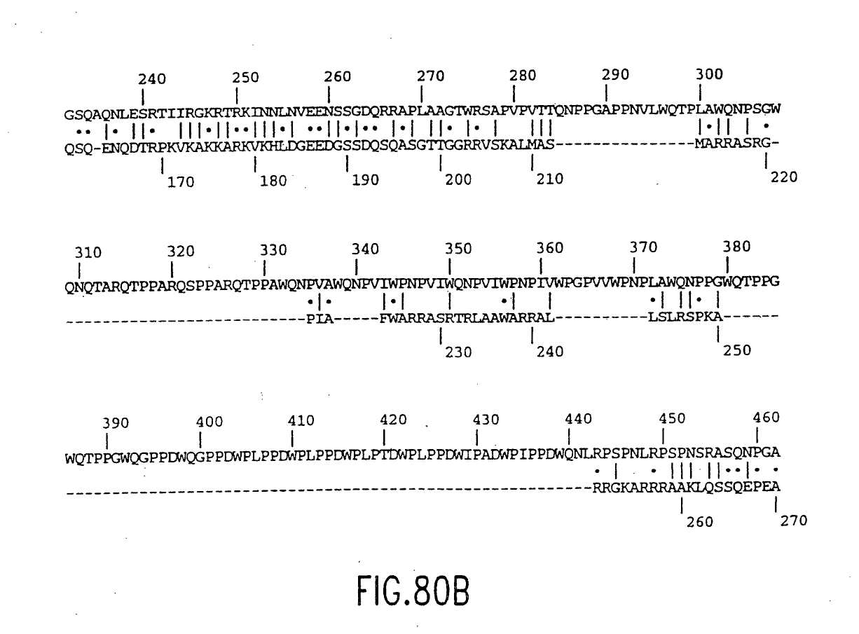

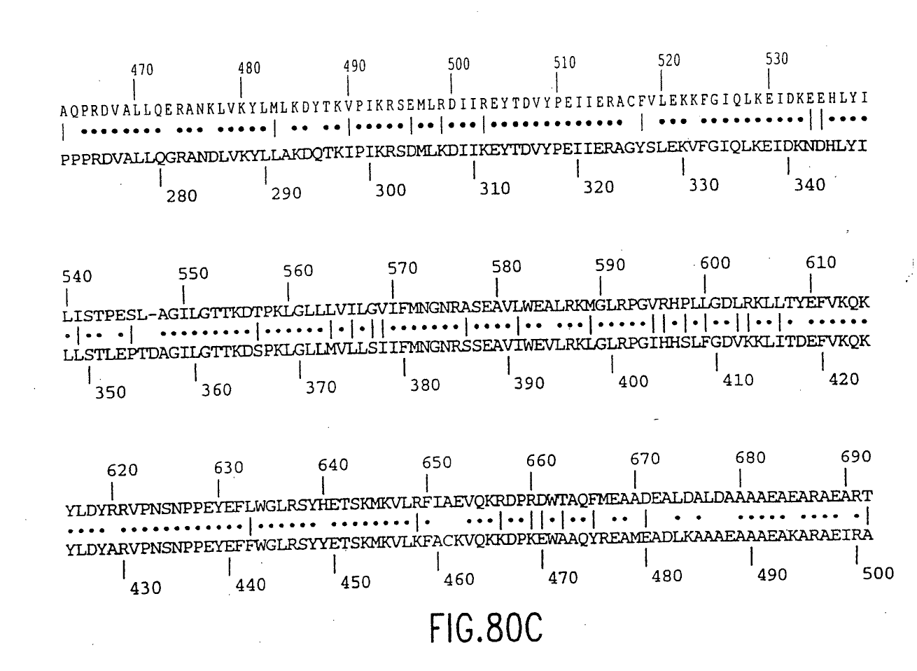

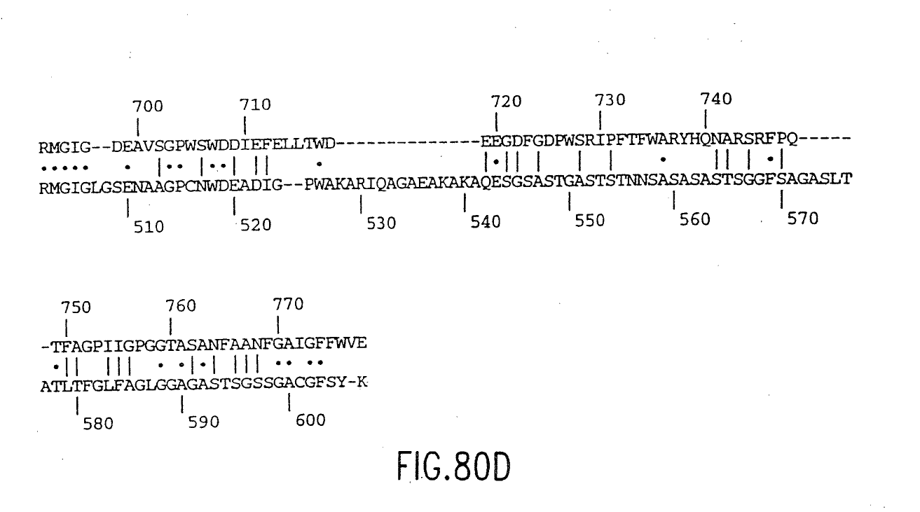

Human TANGO 267 protein bears similarity to a human MAGE-like protein (hepatocellular carcinoma associated gene JCL-1; GenBank Accession Numbers Z98046 and U92544). Human MAGE proteins (Kirkin et al. (1998) APMIS 106:665-79) are melanoma associated antigens recognized by cytotoxic T lymphocytes. It has low immunogenicity. These proteins are potentially useful targets for tumor vaccines. FIG. 80A-80D depicts the alignment of the amino acid sequence of human TANGO 267 and human MAGE-like protein. In this alignment, a (e) between the two sequences indicates an exact match.

### Uses of TANGO 267 Nucleic Acids, Polypeptides, and Modulators Thereof

The TANGO 267 proteins and nucleic acid molecules of the invention have at least one “TANGO 267 activity” (also referred to herein as “TANGO 267 biological activity”). TANGO 267 activity refers to an activity exerted by a TANGO 267 protein or nucleic acid molecule on a TANGO 267 responsive cell in vivo or in vitro. Such TANGO 267 activities include at least one or more of the following activities: 1) interaction of a TANGO 267 protein with a TANGO 267-target molecule; 2) activation of a TANGO 267 target molecule; 3) modulation of cellular proliferation; 4) modulation of cellular differentiation; or 5) modulation of a signaling pathway. Thus, the TANGO 267 proteins, nucleic acids and/or modulators can be used for the treatment of a disorder characterized by aberrant TANGO 267 expression and/or an aberrant TANGO 267 activity, such as proliferative and/or differentiative disorders.

As TANGO 267 was originally discovered in a coronary artery smooth muscle cell by EST analysis, TANGO 267 nucleic acids, proteins, and modulators thereof can be used to treat heart disorders, e.g., ischemic heart disease, atherosclerosis, hypertension, angina pectoris, Hypertrophic Cardiomyopathy, and congenital heart disease.

In another example, because human TANGO 267 protein bears similarity to a human MAGE-like protein (hepatocellular carcinoma associated gene JCL-1), TANGO 267 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

Furthermore, because human TANGO 267 protein bears similarity to a human MAGE-like protein (hepatocellular carcinoma associated gene JCL-1), TANGO 216 polypeptides, nucleic acids and/or modulators thereof can also be used to modulate cell adhesion in proliferative disorders, such as cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosatcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

TANGO 267 could be useful as a target for tumor vaccines. Accordingly, TANGO 25267 proteins (including fragments of TANGO 267) and nucleic acids and/or modulators can be used as tumor vaccines.

### TANGO 253, TANGO 257, and INTERCEPT 258

The TANGO 253, TANGO 257, and INTERCEPT 258 proteins and nucleic acid molecules comprise families of molecules having certain conserved structural and functional features. For example, TANGO 253 proteins, TANGO 257 proteins and INTERCEPT 258 proteins of the invention have signal sequences.

In one embodiment, a TANGO 253 protein contains a signal sequence of about amino acids 1 to 15 or about amino acids 1 to 15 of SEQ ID NO:68. The signal sequence is cleaved during processing of the mature protein.

In another embodiment, a TANGO 257 protein contains a signal sequence of about amino acids 1 to 21 or about amino acids 1 to 21 of SEQ ID NO:72. The signal sequence is cleaved during processing of the mature protein.

In another embodiment, an INTERCEPT 258 protein contains a signal sequence at about amino acids 1 to 29 or about amino acids 1 to 29 of SEQ ID NO:76. The signal sequence is cleaved during processing of the mature protein.

In one embodiment, TANGO 253 includes at least one RGD cell attachment site. An RGD domain contains a contiguous arginine-glycine-aspartic acid amino acid sequence and is involved in cell-cell, cell-extracellular matrix and cell adhesion interactions. In a preferred embodiment, a TANGO 253 family member has the amino acid sequence of SEQ ID NO:68 and, preferably, a RGD cell attachment site is located at about amino acid positions 77 to 79 of SEQ ID NO:68.

TANGO 253 family members can also include a collagen domain. As used herein, the term “collagen domain” refers to a protein domain containing a G-X-Y amino acid repeat motif, wherein the first amino acid residue is glycine and the second and third amino acid residues can be any residue but are preferably proline or hydroxyproline. Typically, a collagen domain contains at least about 3 to 5 G-X-Y repeats, and can contain about 3, 5, 8, 10, 12, 15, 20 or more continuous G-X-Y repeats. In one embodiment, a collagen domain can fold to form a triple helical structure.

In one embodiment, a TANGO 253 family member includes at least one collagen domain having an amino acid sequence that is at least about 40%, 50%, 60%, 70%, 80%, 90%, 95% or 98% identical to amino acids 36 to 95, which is the collagen domain of human TANGO 253, or amino acids 36 to 95, which is the collagen domain of mouse TANGO 253, while maintaining a glycine residue at the first position of G-X-Y repeats within the domain to maintain at least 3, 5, 8, 10, 12, 15 or 20 contiguous G-X-Y repeats, or while most preferably maintaining a glycine repeat at the first position of each G-X-Y repeat within the domain.

TANGO 253 family members can also include a C1q domain or at least one of the conserved amino acid motifs found therein. As used herein, the term “C1q domain” refers to a protein domain that bears homology to a C1q domain present within a member of the C1 enzyme complex. A C1q domain typically includes about 130-140 amino acid residues. C1q domains are utilized in processes involving, e.g., correct protein folding and alignment and protein-protein interactions.

In one embodiment, a TANGO 253 family member includes one or more C1q domains having an amino acid sequence that is at least 45%, preferably about 50%, 55%, 60%, 70%, 75%, 80%, 90%, 95% and most preferably at least about 98% identical to amino acids 105 to 232 of SEQ ID NO:68, which is the human TANGO 253 C1q domain or amino acids 105 to 232 of SEQ ID NO:70, which is the mouse TANGO 253 C1q domain.

Embodiments of TANGO 253 family members include, but are not limited to, human, mouse and rat TANGO 253 nucleic acids and proteins. The features of the human and mouse TANGO 253 are described below. A cDNA encoding a rat TANGO 253 nucleotide sequence, identified in clone jtrxa001e10t1, is 75.4% identical to human TANGO 253 in a 536 bp overlap. Further, the isolated rat TANGO 253 nucleotide sequence is 86% identical to mouse TANGO 253 in a 472 bp overlap.

Embodiments of TANGO 257 family members include, but are not limited to, human, mouse and rat TANGO 257 nucleic acids and proteins. The features of the human and mouse TANGO 257 are described below. A cDNA encoding a rat TANGO 257 nucleotide sequence, identified within clone jtrxa102g06t1, is 83.8% identical to human TANGO 257 in a 734 bp overlap. Further, the isolated rat TANGO 257 nucleotide sequence is 88.4% identical to mouse TANGO 257 in a 731 bp overlap.

In one example, a TANGO 257 family member includes one or more of the following domains: (1) an extracellular domain; (2) a transmembrane domain; and (3) a cytoplasmic domain. In one embodiment, a TANGO 257 protein contains cytoplasmic domains of about amino residues 1 to 202 and about amino acid residues 338 to 406, transmembrane domains of about amino acid residues 203 to 221 and about amino acid residues 321 to 337, and an extracellular domain of about amino acid residues 222 to 320 of SEQ ID NO:72. In an alternative embodiment, a TANGO 257 protein contains an extracellular domain of about amino acid residues 1 to 320 or a mature extracellular domain of about amino acid residues 22 to 320, a transmembrane domain of about amino acid residues 321 to 337, and a cytoplasmic domain of about amino acid residues 338 to 406 of SEQ ID NO:72. In another embodiment, a mature TANGO 257 protein contains about amino acid residues 22 to 406 of SEQ ID NO:72.

In another embodiment, a TANGO 257 protein contains intracellular domains of about amino acid residues 1 to 202 and about amino acid residues 338 to 406, transmembrane domains of about amino acid residues 203 to 221 and about amino acid residues 321 to 337, and an extracellular domain of about amino acid residues 222 to 320 of SEQ ID NO:72. In alternative embodiment, a TANGO 257 protein contains an extracellular domain of about amino acid residues 1 to 320 or a mature extracellular domain of about amino acid residues 22 to 320, a transmembrane domain of about amino acid residues 321 to 337, and an intracellular domain of about amino acid residues 338 to 406 of SEQ ID NO:72. In another embodiment, a mature TANGO 257 protein contains about amino acid residues 22 to 406 of SEQ ID NO:72.

In another example, an INTERCEPT 258 family member includes one or more of the following domains: (1) an extracellular domain; (2) a transmembrane domain; and (3) a cytoplasmic domain. Thus, in one embodiment, an INTERCEPT 258 protein contains extracellular domains of about amino acid residues 1 to 206 or about amino acid residues 30 to 206 and about amino acid residues 272 to 370, transmembrane domains of about amino acid residues 207 to 224 and about amino acid residues 247 to 271, and a cytoplasmic domain of about amino acid residues 225 to 246 of SEQ ID NO:76. In an alternative embodiment, an INTERCEPT 258 protein contains an extracellular domain of about amino acid residues 272 to 370, a transmembrane domain of about amino acid residues 247 to 271, and a cytoplasmic domain of about amino acid residues 1 to 246 or a mature cytoplasmic domain of about amino acid residues 30 to 246 of SEQ ID NO:76. In accordance with these embodiments, an INTERCEPT 258 protein is a mature protein containing an extracellular, transmembrane and cytoplasmic domain of about amino acids 30 to 370 of SEQ ID NO:76.

In another embodiment, an INTERCEPT 258 protein contains an extracellular domain of about amino acids 1 to 249, or a mature extracellular domain of about amino acids 30 to 249 of SEQ ID NO:76. In another embodiment, an INTERCEPT 258 protein contains a transmembrane domain of about amino acids 250 to 274 of SEQ ID NO:76. In another embodiment, an INTERCEPT 258 protein contains a cytoplasmic domain of about amino acids 275 to 394 of SEQ ID NO:76. In accordance with these embodiments, an INTERCEPT 258 protein is a mature protein containing an extracellular, transmembrane and cytoplasmic domain of about 30 to 394 of SEQ ID NO:76.

INTERCEPT 258 family members can also include an immunoglobulin (Ig) domain contained within the extracellular domain. As used herein, the term “Ig domain” refers to a protein domain bearing homology to immunoglobulin superfamily members. An Ig domain includes about 30-90 amino acid residues, preferably about 40-80 amino acid residues, more preferably about 50-70 amino acid residues, still more preferably about 55-65 amino acid residues, and most preferably about 57 to 59 amino acid residues. In certain embodiments, an Ig domain contains a conserved cysteine residue within about 5 to 15 amino acid residues, preferably about 7 to 12 amino acid residues, and most preferably about 8 amino acid residues from its N-terminal end, and another conserved cysteine residue within about 1 to 5 amino acid residues, preferably about 2 to 4 amino acid residues, and most preferably about 3 amino acid residues from its C-terminal end.

An Ig domain typically has the following consensus sequence, beginning about 1 to amino acid residues, more preferably about 3 to 10 amino acid residues, and most preferably about 5 amino acid residues from the C terminal end of the domain: (FY)-Xaa-C-Xaa-(VA)-COO—, wherein (FY) is either a phenylalanine, or a tyrosine residue (preferably tyrosine), where “Xaa” is any amino acid, C is a cysteine residue, (VA) is either a valine or an alanine residue (preferably alanine), and COO— is the protein C terminus.

In one embodiment, an INTERCEPT 258 family member includes one or more Ig domains having an amino acid sequence that is at least about 55%, preferably at least about 565%, more preferably at least 75%, yet more preferably at least about 85%, and most preferably at least about 95% identical to amino acids 49 to 128 and/or amino acids 167 to 226, which are the Ig domains of human INTERCEPT 258.

In another embodiment, an INTERCEPT 258 family member includes one or more Ig domains having an amino acid sequence that is at least about 55%, preferably at least about 65%, more preferably at least about 75%, yet more preferably at least about 85%, and most preferably at least about 95% identical to amino acids 167 to 226, includes a conserved cysteine residue about 8 residues downstream from the N-terminus of the Ig domain, and has one or more Ig domain consensus sequences described herein. In another embodiment, an INTERCEPT 258 family member includes one or more Ig domains having an amino acid sequence that is at least 55%, preferably at least about 65%, more preferably at least about 75%, yet more preferably at least about 85%, and most preferably at least about 95% identical to amino acids 167 to 226 of SEQ ID NO:76, includes a conserved cysteine residue 8 residues downstream from the N-terminus of the Ig domain, has one or more Ig domain consensus sequences described herein, and has a conserved cysteine within the consensus sequence that forms a disulfide both with said first conserved cysteine. In yet another embodiment, an INTERCEPT 258 family member includes one or more Ig domains having an amino acid sequence that is at least 55%, preferably at least about 65%, more preferably at least about 75%, yet more preferably at least about 85%, and most preferably at least about 95% identical to amino acids 167 to 226 of SEQ ID NO:76, includes a conserved cysteine residue 8 residues downstream from the N-terminus of the Ig domain, has one or more Ig domain consensus sequences described herein, has a conserved cysteine within the consensus sequence that forms a disulfide both with said first conserved cysteine, and has at least one INTERCEPT 258 biological activity as described herein.

In a preferred embodiment, an INTERCEPT 258 family member has the amino acid sequence wherein the aforementioned Ig conserved residues are located as follows: the N-terminal conserved cysteine residue is located at about amino acid position 174 and the C-terminal conserved cysteine is located at about amino acid position 224 of SEQ ID NO:76.

In another embodiment, an INTERCEPT 258 family member includes one or more Ig domains having an amino acid sequence that is at least about 55%, preferably at least about 65%, more preferably at least about 75%, yet more preferably at least about 85%, and most preferably at least about 95% identical to amino acids 170 to 229 of SEQ ID NO:76, which is the Ig domain of mouse INTERCEPT 258. In another embodiment, an INTERCEPT 258 family member includes one or more Ig domains having an amino acid sequence that is at least about 55%, preferably at least about 65%, more preferably at least about 75%, yet more preferably at least about 85%, and most preferably at least about 95% identical to amino acids 170 to 229 of SEQ ID NO:76, includes a conserved cysteine residue about 8 residues downstream from the N-terminus of the Ig domain, and has one or more Ig domain consensus sequences described herein, has a conserved cysteine within the consensus sequence that forms a disulfide both with said first conserved cysteine, and has at least one INTERCEPT 258 biological activity as described herein.

In a preferred embodiment, an INTERCEPT 258 family member has the amino acid sequence wherein the aforementioned Ig domain conserved residues are located as follows: the N-terminal conserved cysteine residue is located at about amino acid residue position 177 and the C-terminal conserved cysteine residue is located at about amino acid position 227 of SEQ ID NO:76.

### Human TANGO 253

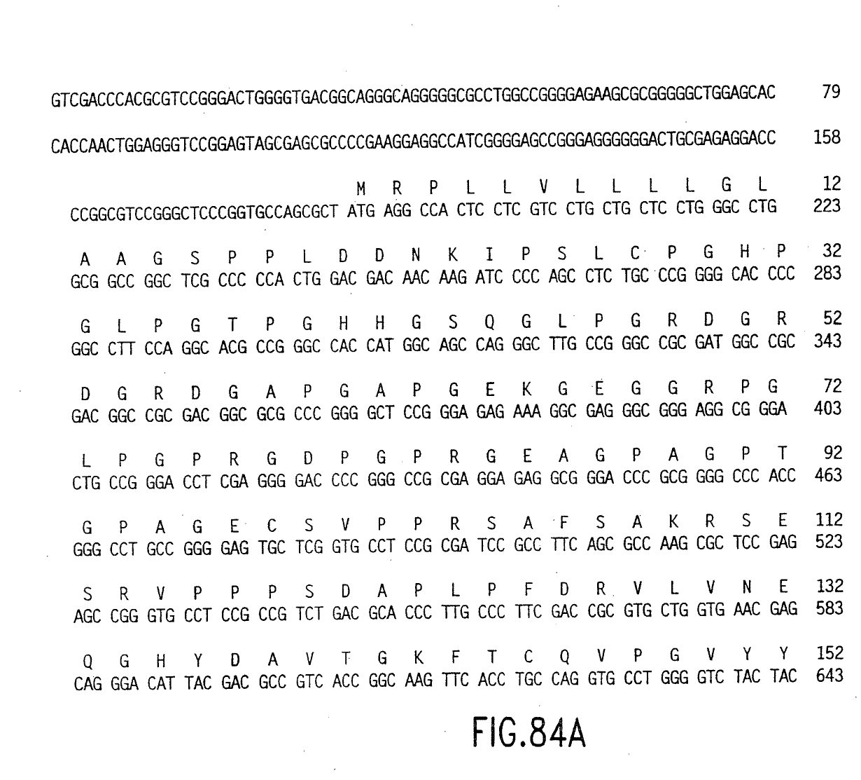

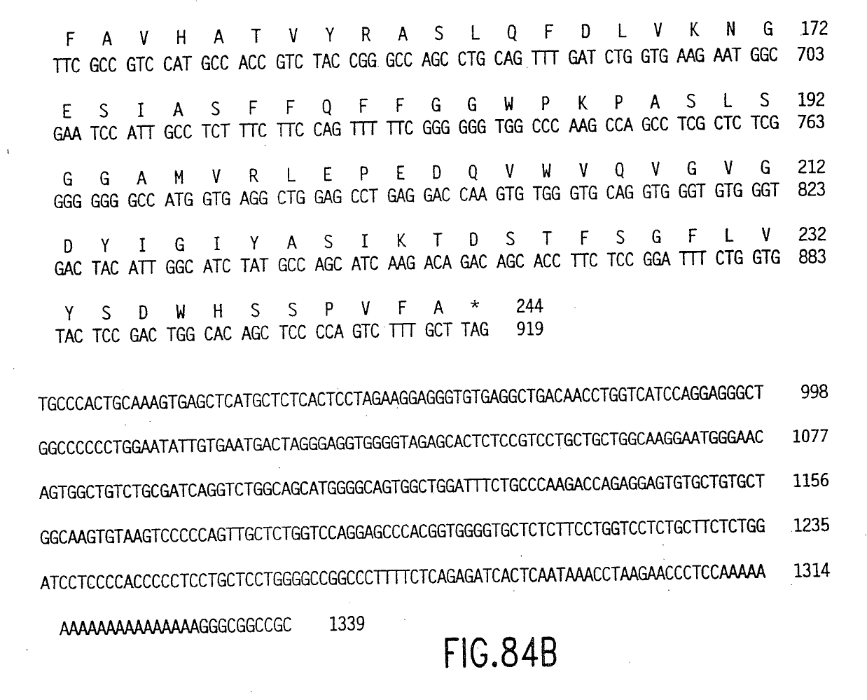

A cDNA encoding human TANGO 253 was identified by analyzing the sequences of clones present in a coronary artery smooth muscle library for sequences that encode secreted proteins. The primary cells utilized in construction of the library had been stimulated with agents that included phorbol 12-myristate 13-acetate (PMA), tumor neurosis factor (TNF), ionomycin, and cyclohexamide (CHX). This analysis led to the identification of a clone, Athma27h9, encoding full-length human TANGO 253. The human TANGO 253 cDNA of this clone is 1339 nucleotides long (FIG. 84A-84B; SEQ ID NO:67). The open reading frame of this cDNA, nucleotides 188 to 916, encodes a 243 amino acid secreted protein (SEQ ID NO:68).

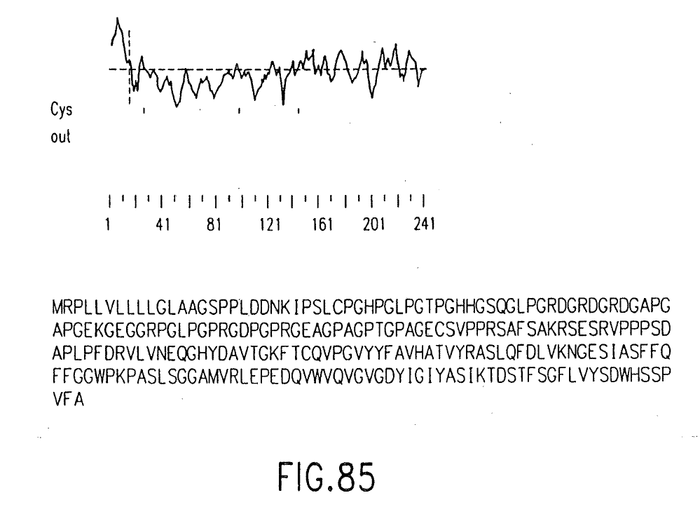

FIG. 85 depicts a hydropathy plot of human TANGO 253. The dashed vertical line separates the signal sequence (amino acids 1 to 15) on the left from the mature protein (amino acids 15 to 243 of SEQ ID NO:68) on the right.

The signal peptide prediction program SIGNALP (Nielsen et al., 1997, Protein Engineering 10:1-6) predicted that human TANGO 253 includes a 15 amino acid signal peptide (amino acid 1 to amino acid 15 of SEQ ID NO:68) preceding the mature human TANGO 253 protein (corresponding to amino acid 16 to amino acid 243 of SEQ ID NO:68). The molecular weight of TANGO 253 protein without post-translational modifications is 25.3 kDa prior to the cleavage of the signal peptide, 23.8 kDa after cleavage of the signal peptide.

Human TANGO 253 includes a collagen domain (at about amino acids 36 to 95) and a C1q domain (at about amino acids 105 to 232) containing 23 G-X-Y repeats. An RGD cell attachment site is found at amino acids 77 to 79.

Three protein kinase C phosphorylation sites are present in human TANGO 253. The first has the sequence SAK (at amino acids 107 to 109), the second has the sequence TGK (at amino acids 140 to 142), and the third has the sequence SIK (at amino acids 220 to 222). Human TANGO 253 has three N-myristylation sites. The first has the sequence GLAAGS (at amino acids 11 to 16), the second has the sequence GGRPGL (at amino acids 68 to 73) and the third has the sequence GIYASI (at amino acids 216 to 221).

Northern analysis of human TANGO 253 expression demonstrates strong expression in heart, lung, liver, kidney and pancreas, and moderate expression in brain, placenta and skeletal muscle. Liver expression reveals two human TANGO mRNA bands, one of approximately 1.3 kb (which is the size observed in the other tissues) as well as a band at approximately 1 kb, which may be the result of an alternative splicing event.

Secretion assays reveal a human TANGO 253 protein of approximately 30 kDa. The secretion assays were performed as follows: 8×10⁵ 293T cells were plated per well in a 6-well plate and the cells were incubated in growth medium (DMEM, 10% fetal bovine serum, penicillin/strepomycin) at 37° C., 5% CO₂ overnight. 293T cells were transfected with 2 μg of full-length TANGO 253 inserted in the pMET7 vector/well and 10 μg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16-424-54). 1 ml DMEM without methionine and cysteine with 50 μCi Trans-³⁵S (ICN Cat. # 51006) was added to each well and the cells were incubated at 37° C., 5% CO₂ for the appropriate time period. A 150 μl aliquot of conditioned medium was obtained and 150 μl of 2×SDS sample buffer was added to the aliquot. The sample was heat-inactivated and loaded on a 4-20% SDS-PAGE gel. The gel was fixed and the presence of secreted protein was detected by autoradiography.

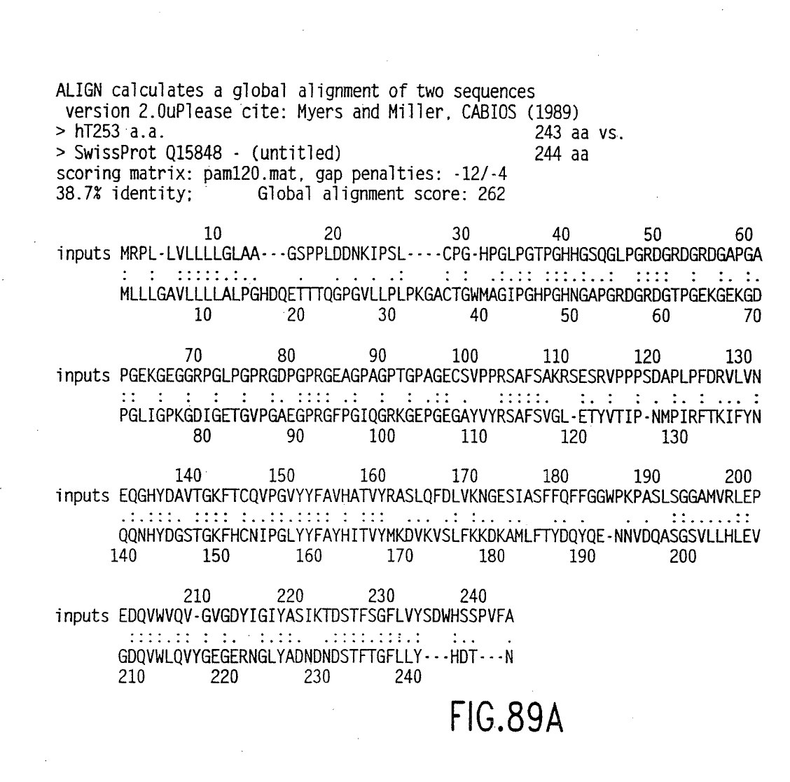

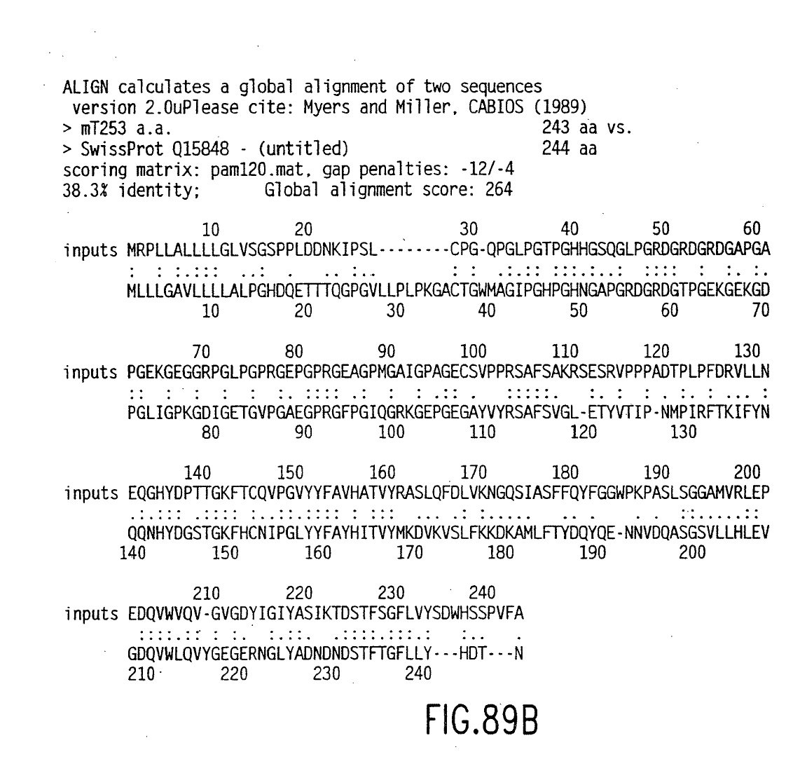

TANGO 253 exhibits homology to an adipocyte complement-mediated protein precursor and so may be involved in adipocyte function, e.g., may act as a signaling molecule for adipocyte tissue. FIG. 89A-89B shows an alignment of the human TANGO 253 amino acid sequence with the human adipocyte complement-mediated protein precursor amino acid sequence. The alignment shows that there is a 38.7% overall amino acid sequence identity between human TANGO 253 and human adipocyte complement-mediated protein precursor.

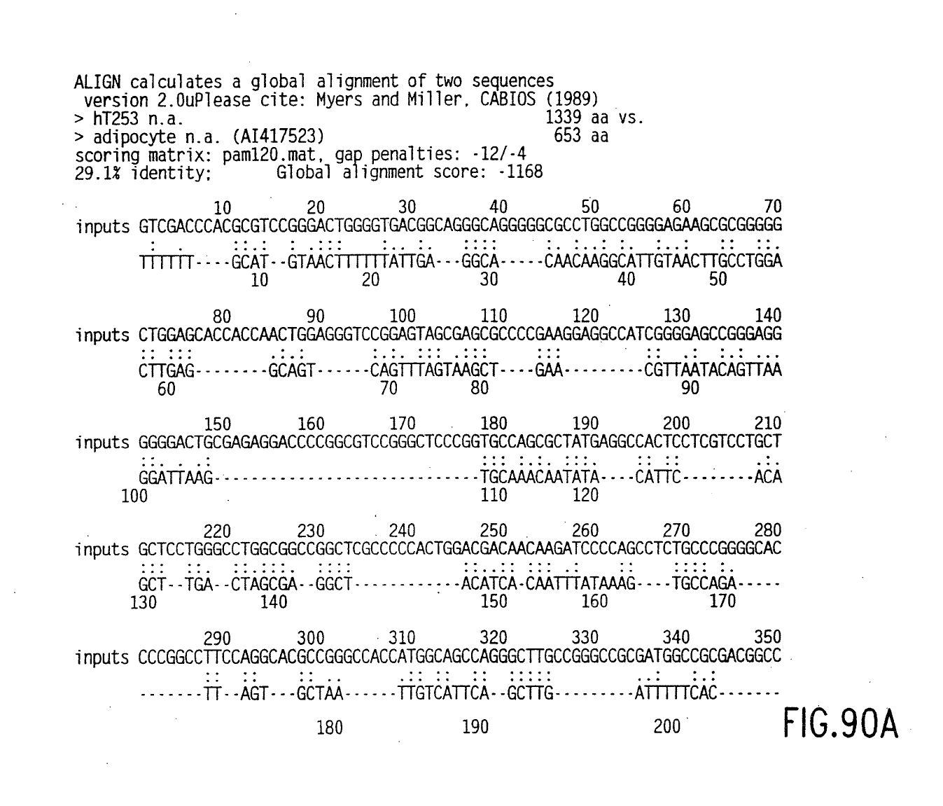

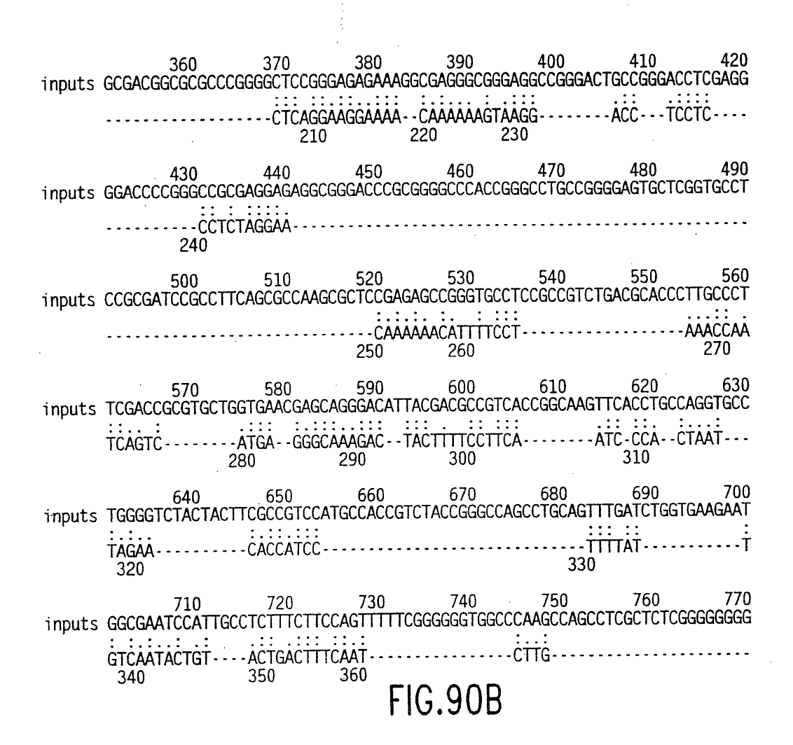

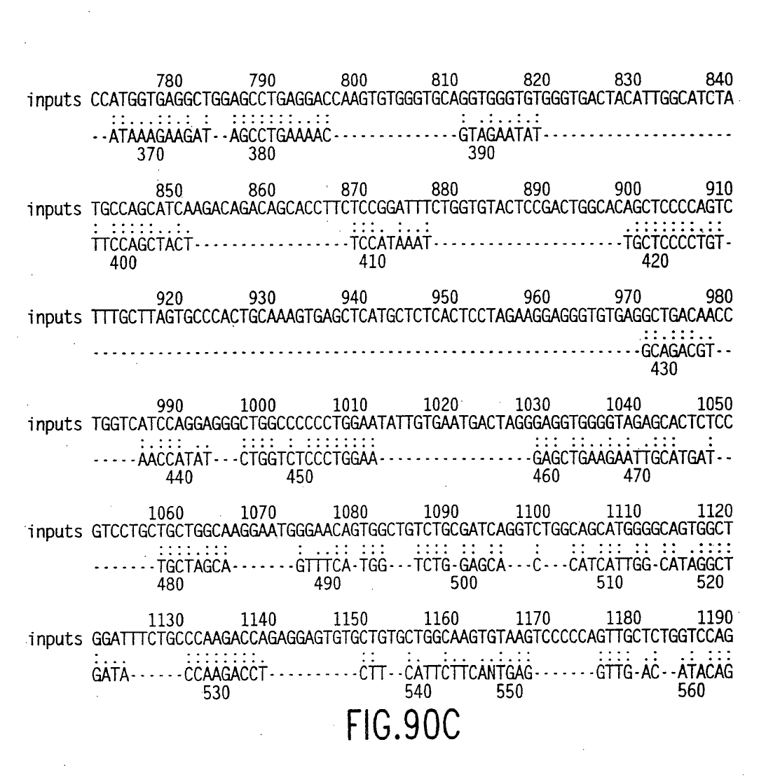

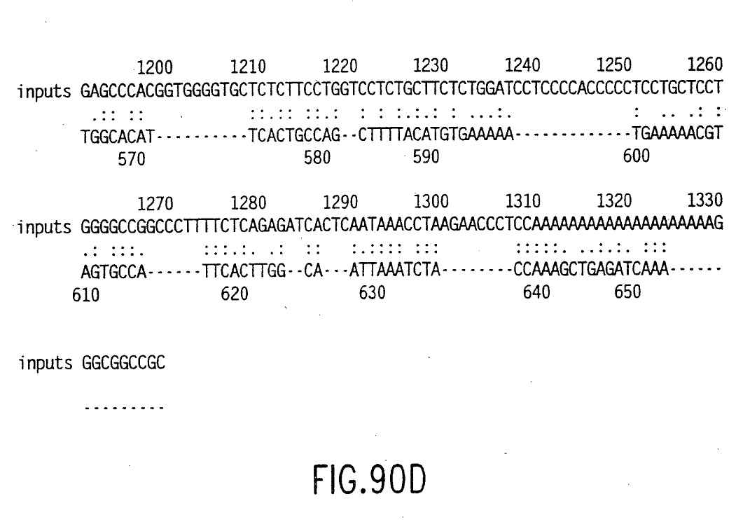

FIG. 90A-90D shows an alignment of the nucleotide sequence of human adipocyte complement-mediated protein precursor nucleotide sequence; GenBank Accession Number A1417523) and the nucleotide sequence of human TANGO 253. The alignment shows a 29.1% overall sequence identity between the two nucleotide sequences.

The human TANGO 253 nucleotide sequence was mapped to human chromosome 11, between flanking markers D11S1356 and D11S924 using the Genebridge 4 Human Radiation hybrid mapping panel with CAAAGTGAGCTCATGCTCTCAC as the forward primer and CTCTGGTCTTGGGCAGAAATC as the reverse primer.

Clone EpT253, which encodes human TANGO 253, was deposited with the American Type Culture Collection (10801 University Boulevard, Manassas, Va. 20110-2209) on Apr. 21, 1999 and assigned Accession Number 207222. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Mouse TANGO 253

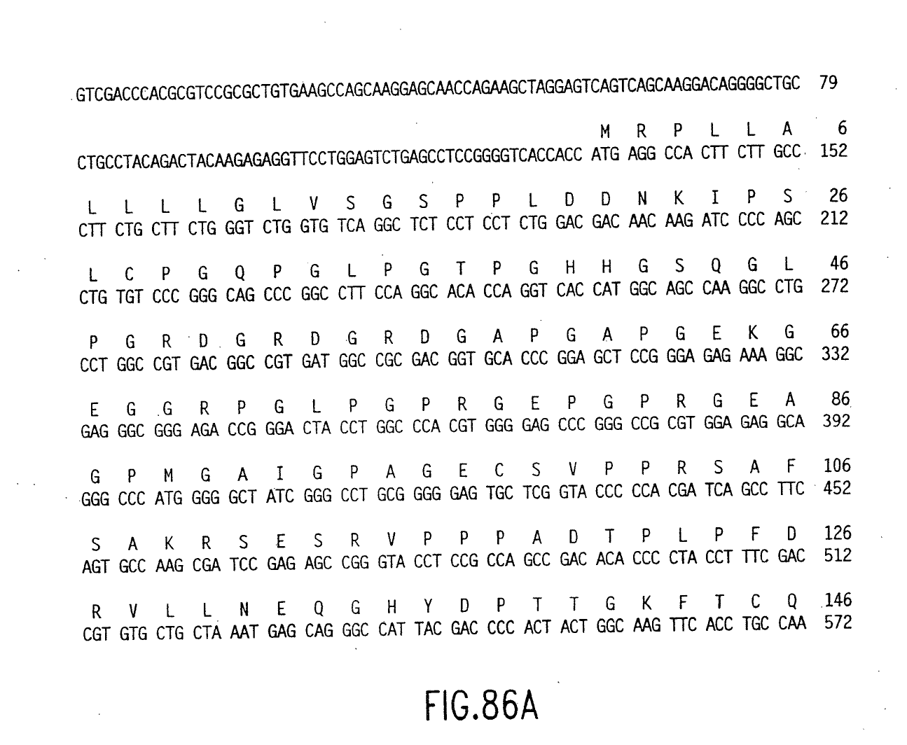

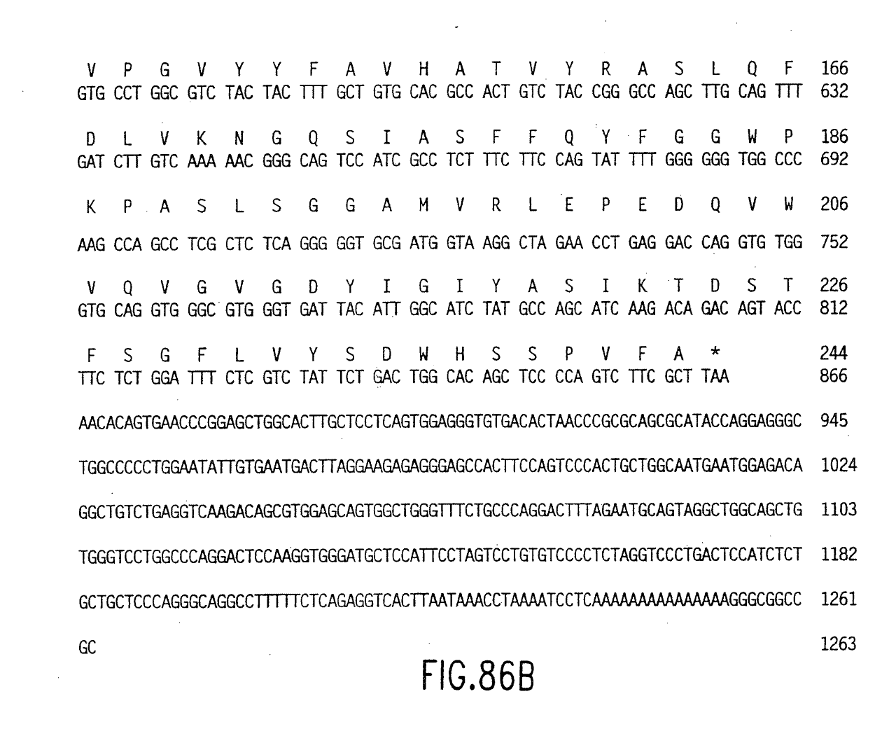

A cDNA encoding mouse TANGO 253 was identified by analyzing the sequences of clones present in a mouse microglia library using a rat TANGO 253 probe from sciatic nerve. This analysis led to the identification of a clone, AtmXale1075, encoding full-length mouse TANGO 253. The mouse TANGO 253 cDNA of this clone is 1263 nucleotides long (FIG. 86A-86B; SEQ ID. NO:69). The open reading frame of this cDNA (nucleotides 135 to 863 of SEQ ID NO:69) encodes a 243 amino acid secreted protein (SEQ ID NO:70).

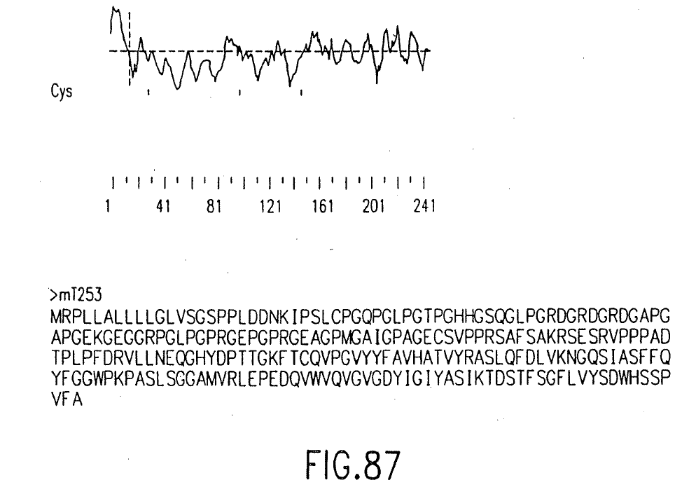

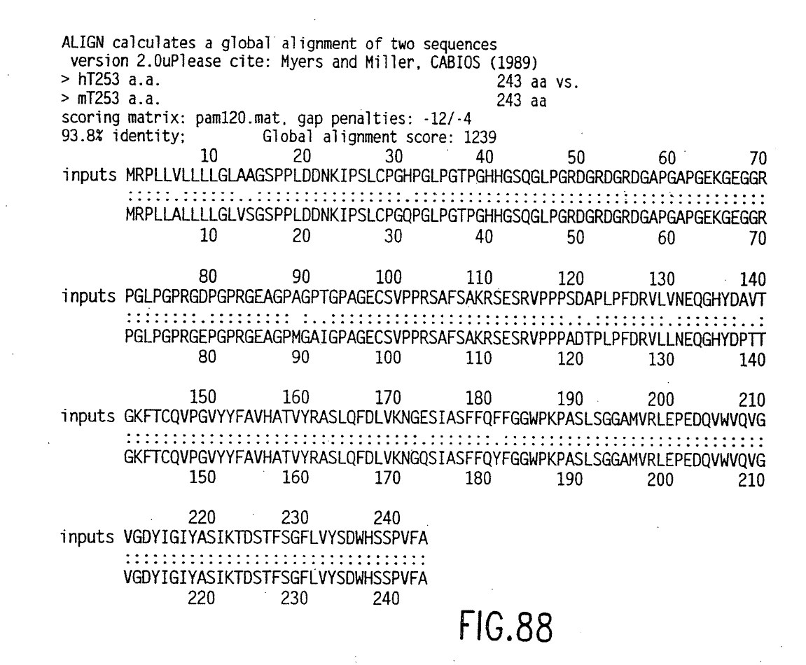

FIG. 87 depicts a hydropathy plot of mouse TANGO 253. The dashed vertical line separates the signal sequence (amino acid 1 to amino acid 15 of SEQ ID NO:70) on the left from the mature protein (amino acid 16 to amino acid 243 of SEQ ID NO:70) on the right.

The signal peptide prediction program SIGNALP (Nielsen et al., 1997, Protein Engineering 10:1-6) predicted that mouse TANGO 253 includes a 15 amino acid signal peptide (amino acid 1 to amino acid 15 of SEQ ID NO:70) preceding the mature mouse TANGO 253 protein (corresponding to amino acid 16 to amino acid 243 of SEQ ID NO:70). The molecular weight of mouse TANGO 253 protein without post-translational modifications is 25.4 kDa prior to the cleavage of the signal peptide, 23.9 kDa after cleavage of the signal peptide.

Mouse TANGO 253 includes a collagen domain (at amino acids 36 to 95) and a C1q domain (at amino acids 105-232).

Three protein kinase C phosphorylation sites are present in mouse TANGO 253. The first has the sequence SAK (at amino acids 107 to 109), the second has the sequence TGK (at amino acids 140 to 142), and the third has the sequence SIK (at amino acids 220 to 222). Mouse TANGO 253 has four N-myristylation sites. The first has the sequence GLVSGS (at amino acids 11 to 16), the second has the sequence GGRPGL (at amino acids 68 to 73), the third has the sequence GQSIAS (at amino acids 172 to 177), and the fourth has the sequence GIYASI (at amino acids 216 to 221).

As shown in FIG. 5A-5B, human TANGO 253 protein and mouse TANGO 253 protein are 93.8% identical. FIG. 89B shows an alignment of the mouse TANGO 253 amino acid sequence with the human adipocyte complement-mediated protein precursor amino acid sequence. The alignment shows that there is a 38.3% overall amino acid sequence identity between mouse TANGO 253 and human adipocyte complement-mediated protein precursor.

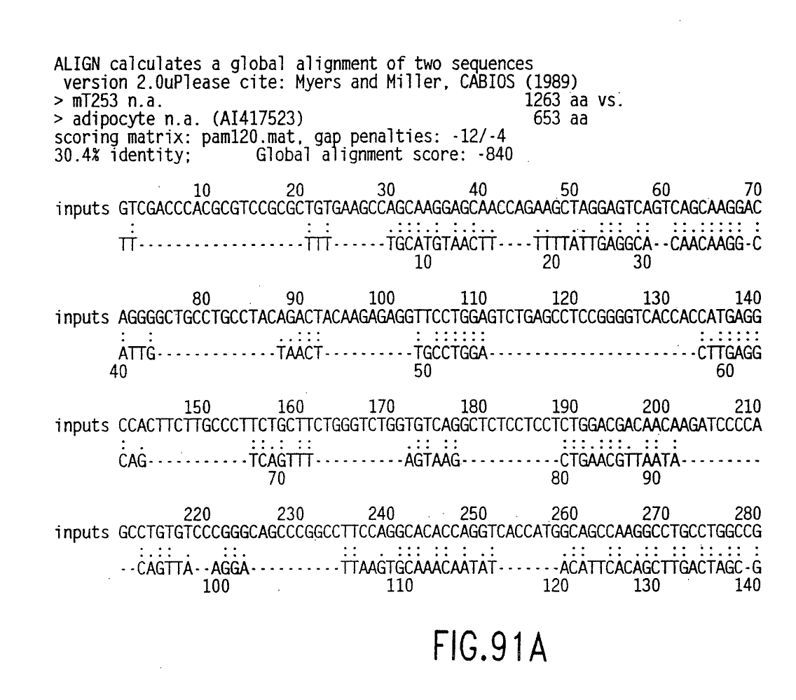

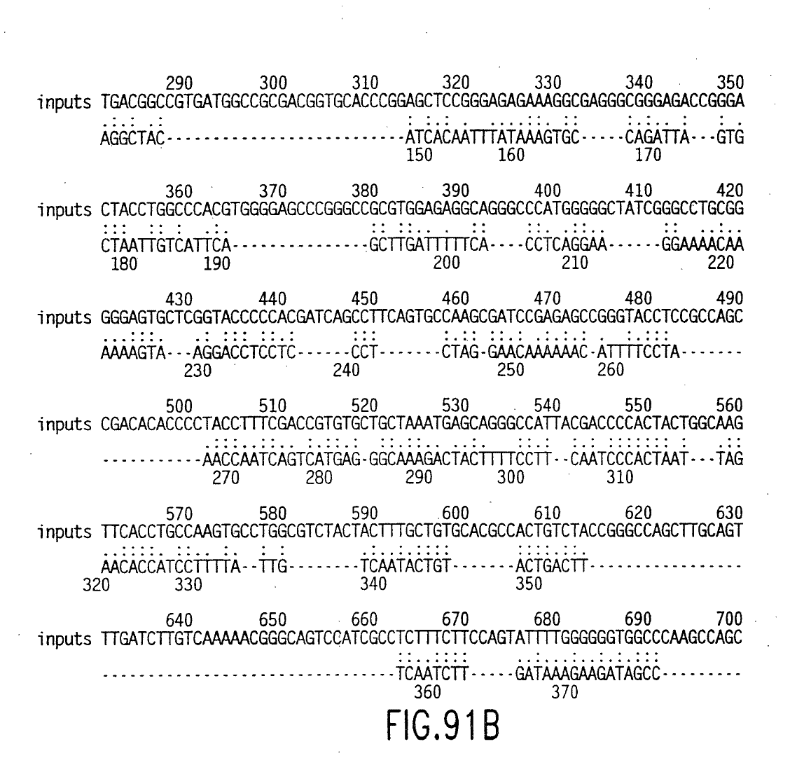

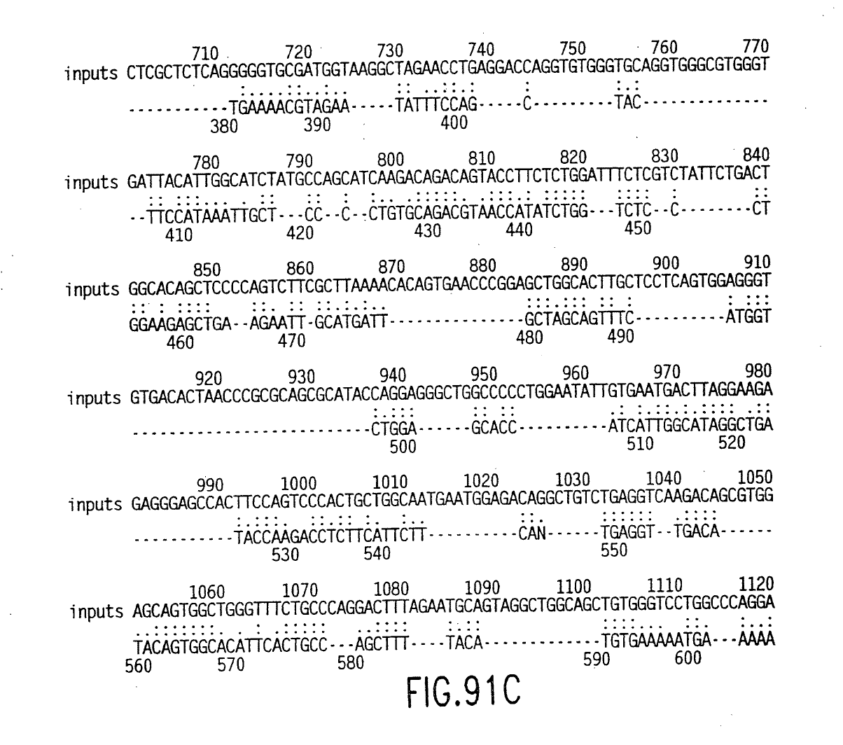

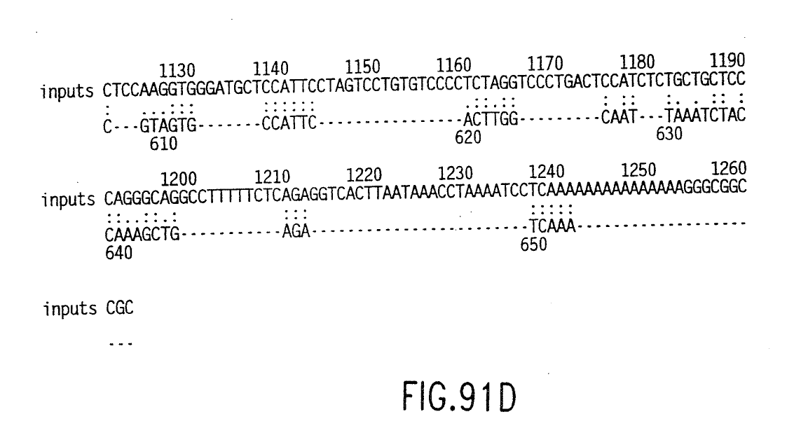

FIG. 91A-91D shows an alignment of the nucleotide sequence of human adipocyte complement-mediated protein precursor nucleotide sequence; GenBank Accession Number A1417523) and the nucleotide sequence of mouse TANGO 253. The alignment shows a 30.4% overall sequence identity between the two nucleotide sequences.

In situ tissue screening was performed on mouse embryonic tissue (obtained from embryos at embryonic day 13.5 to postnatal day 1.5) and adult tissue to determine the expression of mouse TANGO 253 mRNA. Expression of mouse TANGO 253 during embryogenesis was ubiquitously expressed throughout the central nervous system. Strong expression of mouse TANGO 253 was detected in choriod plexus of the fourth ventricle of E18.5 and E1.5 embryos examined. Expression of mouse TANGO 253 was also detected in the lungs of E14.5 and E15.5 embryos and in the kidneys of E15.5 embryos.

Mouse TANGO 253 expression was detected by in situ hybridization in the following adult tissues: a signal was detected in the brain in the choroid plexus of the lateral and 4th ventricles, and the olfactory bulb; a signal was detected in the cortical region of the kidney consistent with the pattern of glomeruli (in particular, the cortical radial veins); a ubiquitous signal was detected in the thymus; a weak, ubiquitous signal was detected in the spleen; a moderate signal was associated with the seminiferous vesicles of the testes; a signal was detected in the ovaries; and a ubiquitous signal restricted to the zone of giant cells was detected in the placenta.

Clone EpTm253, which encodes mouse TANGO 253, was deposited with the American Type Culture Collection (10801 University Boulevard, Manassas, Va. 20110-2209) on Apr. 21, 1999 and assigned Accession Number 207215. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 253 Nucleic Acids, Polypeptides, and Modulators Thereof

As TANGO 253 was originally found in the coronary artery smooth muscle library described above, TANGO 253 nucleic acids, proteins, and modulators thereof can be used to modulate the proliferation, development, differentiation, and/or function of organs, e.g., tissues and cells that form blood vessels and coronary tissue, e.g., cells of the coronary connective tissue, e.g., abnormal coronary smooth muscle cells and/or endothelial cells of blood vessels. TANGO 253 nucleic acids, proteins, and modulators thereof can also be used to modulate symptoms associated with abnormal coronary function, e.g., heart diseases and disorders such as atherosclerosis, coronary artery disease and plaque formation.

In light of the collagen domain, TANGO 253 nucleic acids, proteins and modulators thereof can be utilized to modulate (e.g., stabilize, promote, inhibit or disrupt) cell/extracellular matrix (ECM) interactions, cell/cell interactions and, for example, signal transduction events associated with such interactions. For example, such TANGO 253 compositions and modulators thereof can be used to modulate binding of such ECM-associated factors as integrin and can function to modulate ligand binding to cell surface receptors. In addition, TANGO 253 nucleic acids, proteins and modulators thereof can be utilized to modulate connective tissue formation, maintenance and function, as well as to modulate symptoms associated with connective tissue-related disorders, to promote wound healing, and to reduce, slow or inhibit ameliorate connective tissue-related signs of aging, such as wrinkle formation.

In light of the Clq domain exhibited by TANGO 253 proteins and their similarity to the collectin family, TANGO 253 nucleic acids, proteins and modulators thereof can be utilized to modulate immune-related processes such as the ability to modulate host immune response by, e.g., modulating one or more elements in the serum complement cascade, including, for example activation of the cascade, formation of and/or binding to immune complexes, detection and defense against surface antigens and bacteria, and immune surveillance for rapid removal or pathogens. Such TANGO 253 compositions and modulators thereof can be utilized, e.g., to ameliorate incidence of any symptoms associated with disorders that involve such immune-related processes, including, but not limited to infection and autoimmune disorders.

In addition, such compositions and modulators thereof can be utilized to modulate folding and alignment of the collagen domain (e.g., into a triple helix), disorders associated with collagen defects, including but not limited to bone disorders, e.g., bone resorption disorders, or hearing, e.g., inner ear, disorders, to modulate protein-protein interactions and recognition events (either homotypic or heterotypic) and cellular response events (e.g., signal transduction events) associated with such interactions and recognitions, and to ameliorate symptoms associated with abnormal signaling, protein-protein interaction and/or cellular response events including, but not limited to cell proliferation disorders such as cancer, abnormal neuronal interactions, such as disorders involving abnormal synaptic activity, e.g., abnormal Purkinje cell activities.

Human TANGO 253 protein contains an RGD domain. As such, TANGO 253 nucleic acids, proteins and modulators thereof can be utilized to modulate processes involved in, e.g., bone development, sepsis, tumor progression, metastasis, cell migration, fertilization, and cellular interactions with the extracellular matrix required for growth, differentiation, and apoptosis, as well as cellular processes involving cell adhesion, such as cell migration.

TANGO 253 proteins exhibit similarity to adipocyte complement-related protein precursor and can act as signaling molecules for adipocyte tissue. In light of this, TANGO 253 nucleic acids, proteins and modulators thereof can be utilized to modulate adipocyte function and adipocyte-related processes and disorders such as, e.g., obesity.

TANGO 253 nucleic acids, proteins, and modulators thereof can also be utilized to modulate the development, differentiation, maturation, proliferation and/or activity of cells of the central nervous system such as neurons, glial cells (e.g., astrocytes and oligodendrocytes), and Schwann cells. TANGO 253 nucleic acids, polypeptides, or modulators thereof can also be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), tumors (e.g., neuroglial tumors, neuronal tumors, tumors of pineal cells, meningeal tumors, primary and secondary lymphomas, intracranial tumors, and medulloblastoma), and to treat injury or trauma to the brain.

TANGO 253 nucleic acids, proteins, and modulators thereof can also be utilized to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy), acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

TANGO 253 nucleic acids, proteins and modulators thereof can, in addition to the above, be utilized to regulate or modulate development and/or differentiation of processes involved in microglial, lung, liver, kidney, pancreas, brain, placental and skeletal muscle formation and activity, as well as in ameliorating any symptom associated with a disorder of such cell types, tissues and organs.

TANGO 253 expression can be utilized as a marker (e.g., an in situ marker) for specific tissues (e.g., the brain) and/or cells (e.g., neurons) in which TANGO 253 is expressed. TANGO 253 nucleic acids can also be utilized for chromosomal mapping.

### Human TANGO 257

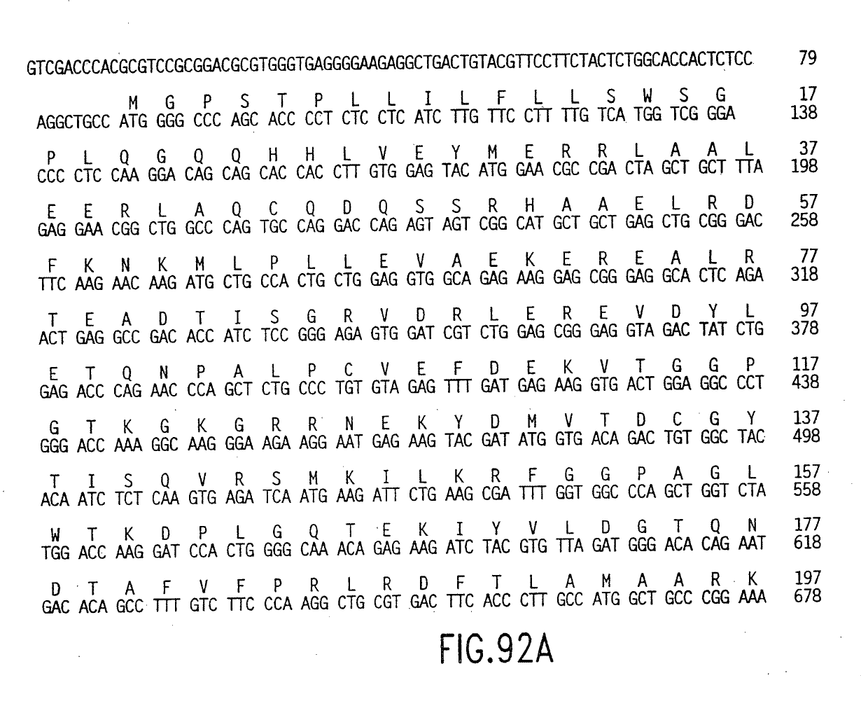

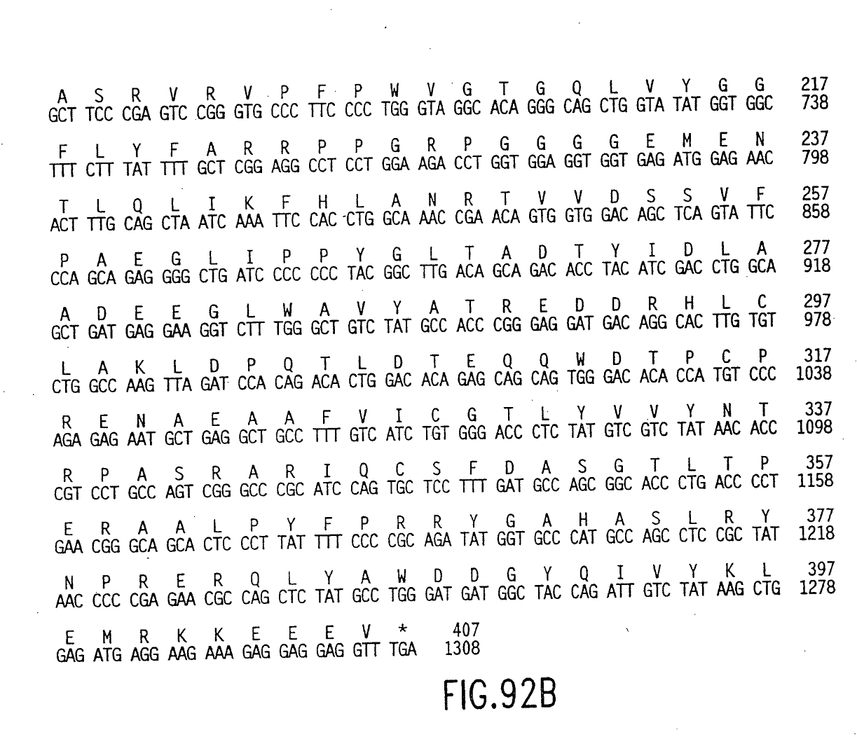

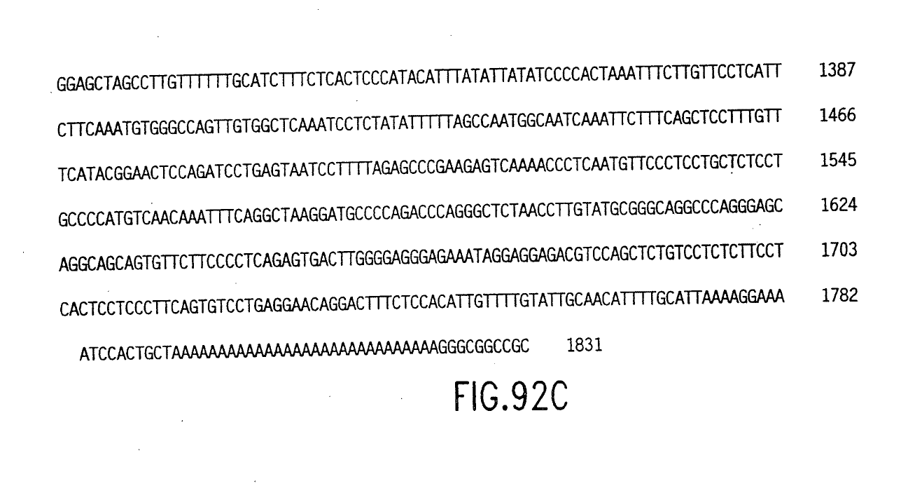

A cDNA encoding human TANGO 257 was identified by analyzing the sequences of clones present in a coronary smooth muscle library for sequences that encode secreted proteins. This analysis led to the identification of a clone, Athma7c10, encoding full-length human TANGO 257. The human TANGO 257 cDNA of this clone is 1832 nucleotides long (FIG. 92A-92C; SEQ ID NO:71). The open reading frame of this cDNA, nucleotides 88 to 1305, encodes a 406 amino acid secreted protein (SEQ ID NO:72).

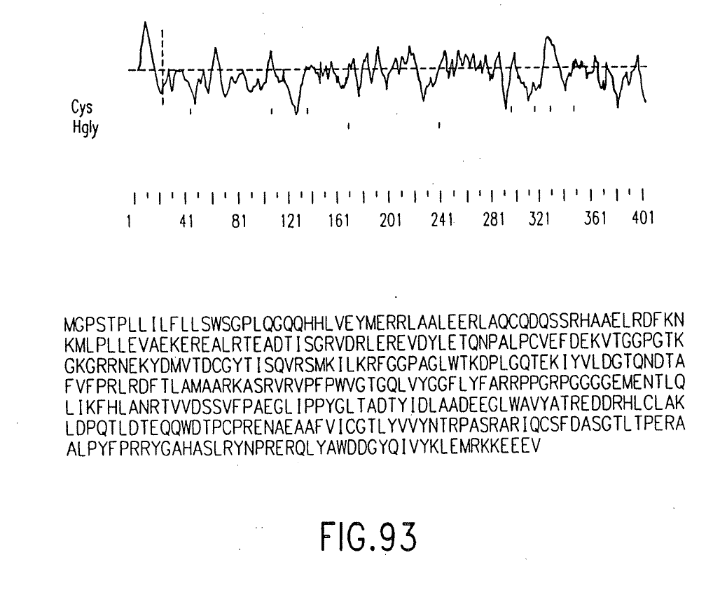

FIG. 93 depicts a hydropathy plot of human TANGO 257.

The signal peptide prediction program SIGNALP (Nielsen et al., 1997, Protein Engineering 10:1-6) predicted that human TANGO 257 includes a 21 amino acid signal peptide (amino acid 1 to amino acid 21) preceding the mature human TANGO 257 protein (corresponding to amino acid 22 to amino acid 406). The molecular weight of human TANGO 257 protein without post-translational modifications is 46.0 kDa prior to the cleavage of the signal peptide, 43.8 kDa after cleavage of the signal peptide.

Two N-glycosylation sites are present in human TANGO 257. The first has the sequence NDTA and is found at amino acids 177 to 180, and the second has the sequence NRTV and is found at amino acids 248 to 251. A cAMP and cGMP dependent protein kinase phosphorylation site having the sequence RKAS is found in human TANGO 257 at amino acids 196 to 199. Five protein kinase C phosphorylation sites are present in human TANGO 257. The first has the sequence SSR (at amino acids 48 to 50), the second has the sequence SGR (at amino acids 84 to 86), the third has the sequence SMK (at amino acids 144 to 146), the fourth has the sequence TEK (at amino acids 166 to 168) and the fifth has the sequence SLR (at amino acids 374 to 376). Five casein kinase II phosphorylation sites are present in human TANGO 257. The first has the sequence TEAD (at amino acids 78 to 81), the second has the sequence TQND (at amino acids 175 to 178), the third has the sequence TVVD (at amino acids 250 to 253), the fourth has the sequence TYID (at amino acids 272 to 275), and the fifth has the sequence TRED (at amino acids 289 to 292). Human TANGO 257 has a tyrosine kinase phosphorylation site having the sequence RLEREVDY at amino acids 89 to 96). Human TANGO 257 has three N-myristylation sites. The first has the sequence GGPGTK (at amino acids 115 to 120), the second has the sequence GGPAGL (at amino acids 152 to 157) and the third has the sequence GAHASL (at amino acids 370 to 375). Human TANGO 257 has an amidation site having the sequence KGRR at amino acids 122 to 125.

Northern analysis of human TANGO 257 expression demonstrates moderate expression in heart, liver and pancreas, and low expression in kidney, lung and skeletal muscle.

Secretion assays reveal a human TANGO 257 protein of approximately 50 kDa. The secretion assays were performed as described in the human TANGO 253 section above.

The human TANGO 257 nucleotide sequence was mapped to human chromosome 1 using the Genebridge 4 Human Radiation hybrid mapping panel with GGATGATGG CTACCAGATTGTC as the forward primer and GGAACATTGAGGGTTTTGACTC as the reverse primer.

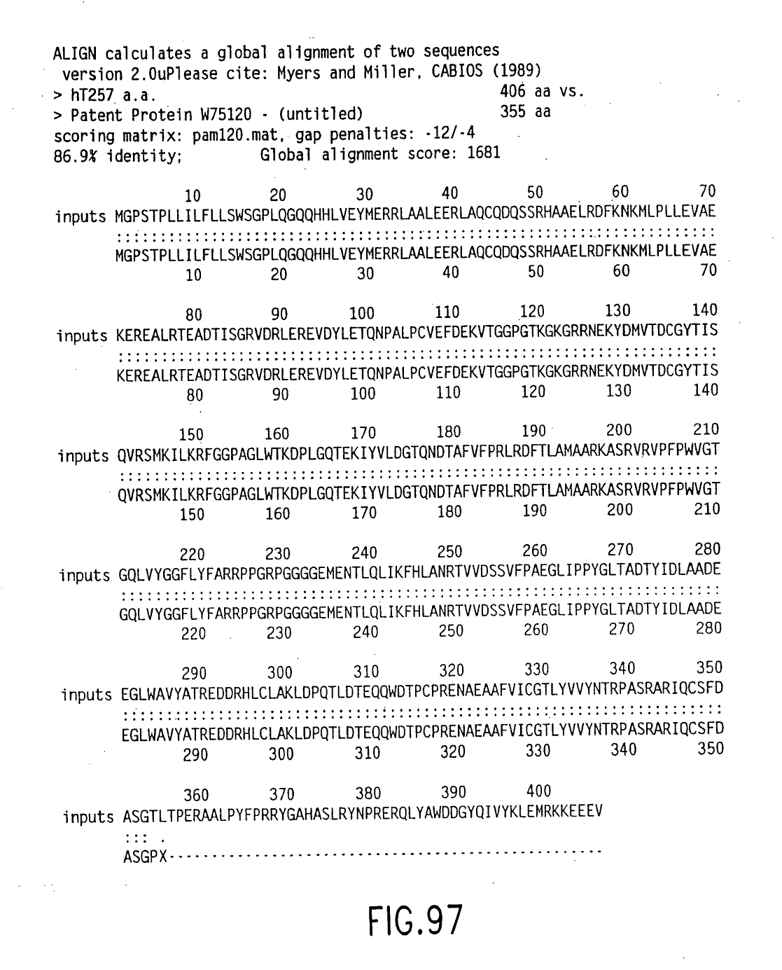

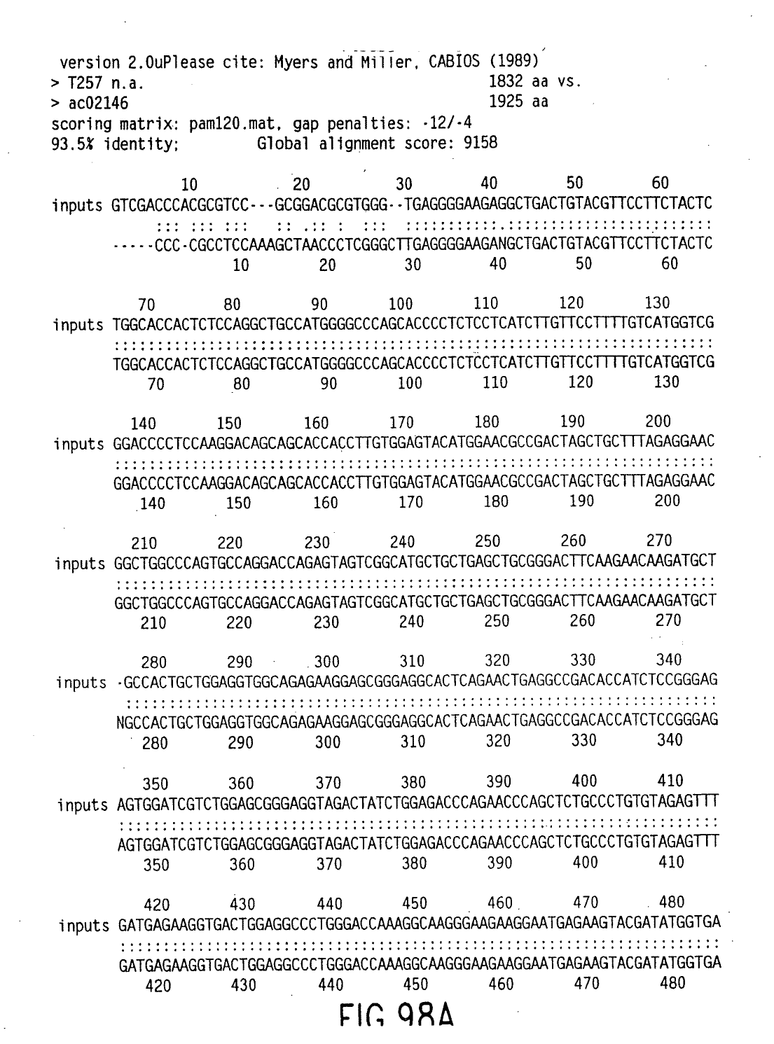

TANGO 257 is homologous to a protein encoded by a nucleic acid sequence referred to in PCT Publication WO 98/39446 as “gene 64”. FIG. 97 shows an alignment of the human TANGO 257 amino acid sequence with the gene 64 encoded amino acid sequence. As shown in the FIGURE, the 353 amino acid gene 64 polypeptide is identical to amino acid residues 1-353 of human TANGO 257. Human TANGO 257 contains 406 amino acids, i.e., contains an additional 53 amino acid residues carboxy to residue 353. The overall amino acid sequence identity between full-length human TANGO 257 polypeptide and the gene 64-encoded polypeptide is approximately 87%.

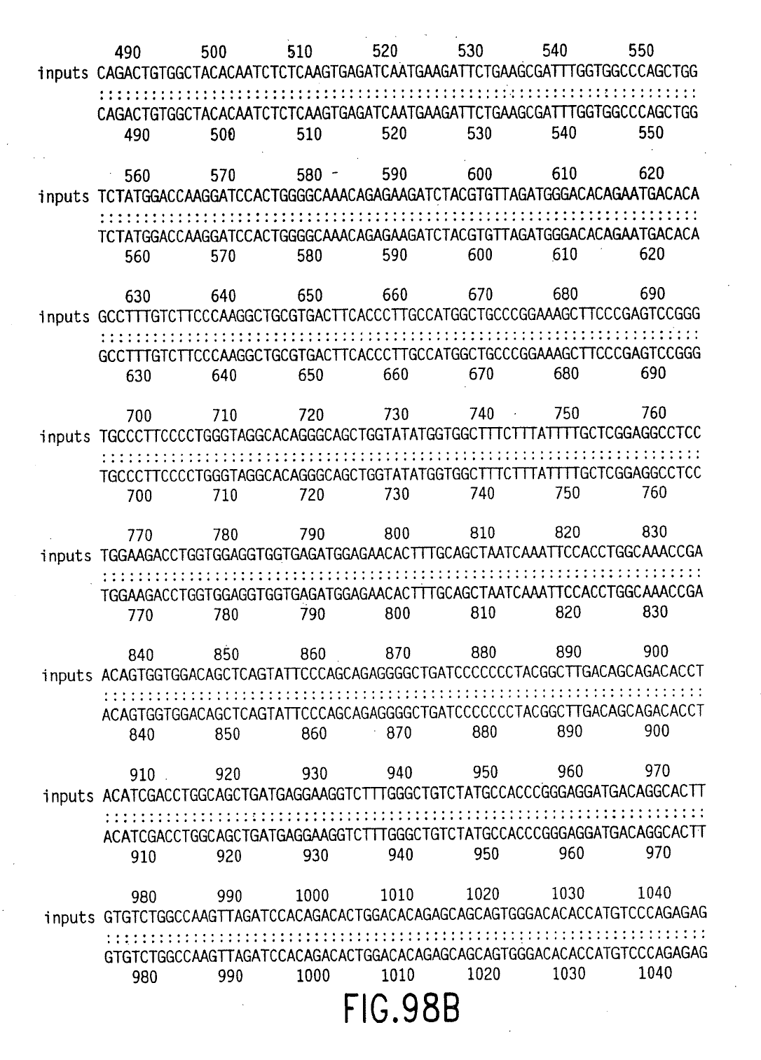

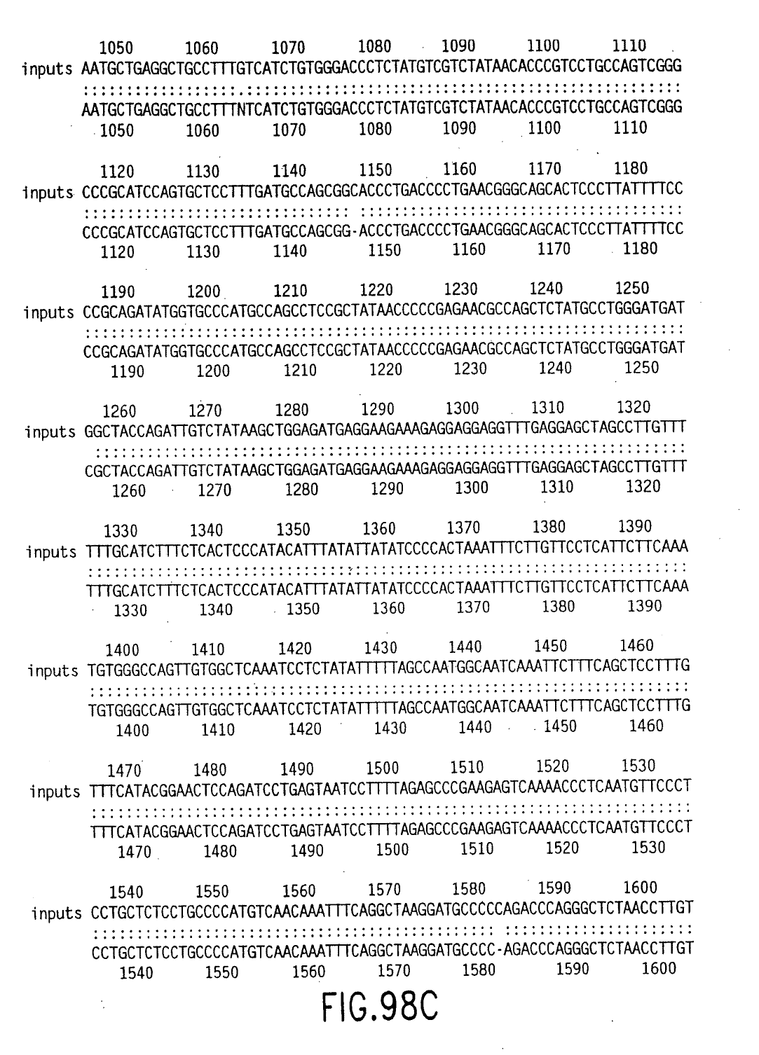

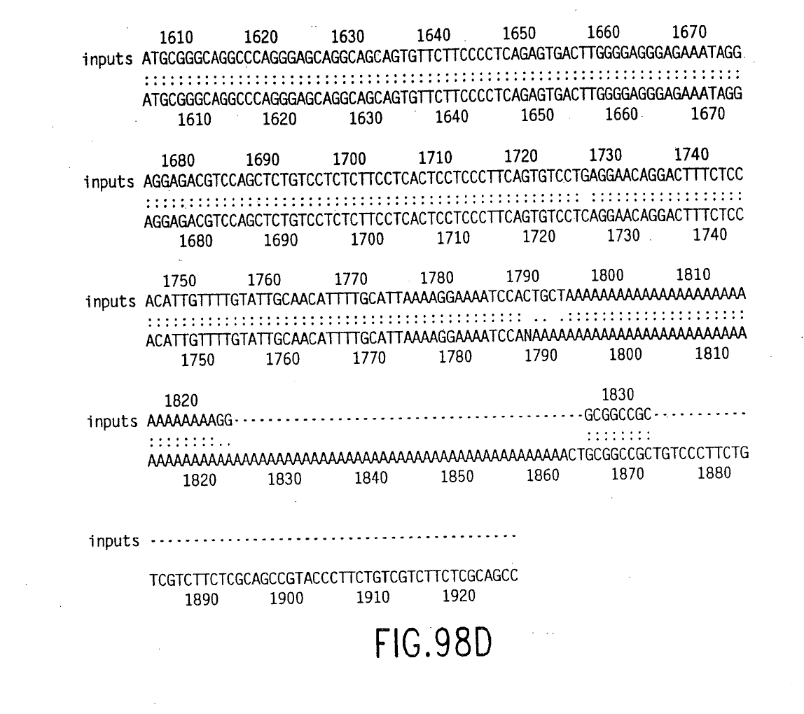

FIG. 98A-98D show an alignment of the nucleotide sequence of gene 64 (PCT Publication WO 98/39446) and the nucleotide sequence of human TANGO 257. The nucleotide sequences of gene 64 and human TANGO 257 are 93.5% identical. Among the differences between the sequences is a cytosine nucleotide at human TANGO 257 position 1587 that represents an insertion relative to the corresponding gene 64 position when the gene 64 and TANGO 257 sequences are aligned. This additional cytosine results in the TANGO 257 open reading frame being 1218 base pairs encoding a polypeptide of 406 amino acid residues. In contrast, the gene 64 nucleic acid sequence encodes a polypeptide of only 353 amino acid residues, as discussed above.

Clone EpT257, which encodes human TANGO 257, was deposited with the American Type Culture Collection (10801 University Boulevard, Manassas, Va. 20110-2209) on Apr. 21, 1999 and assigned Accession Number 207222. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Mouse TANGO 257

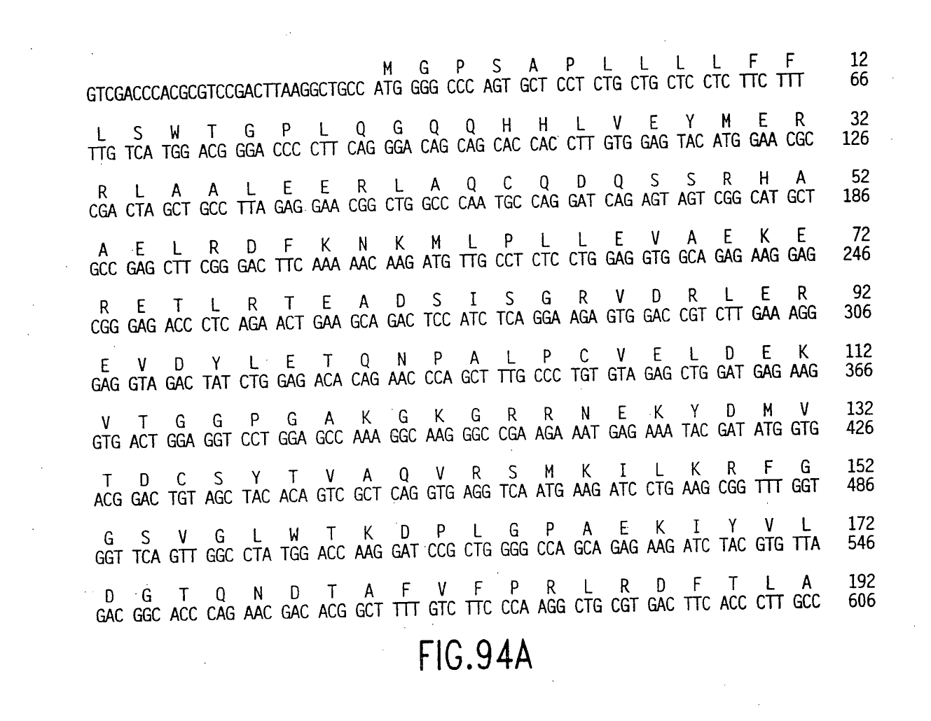

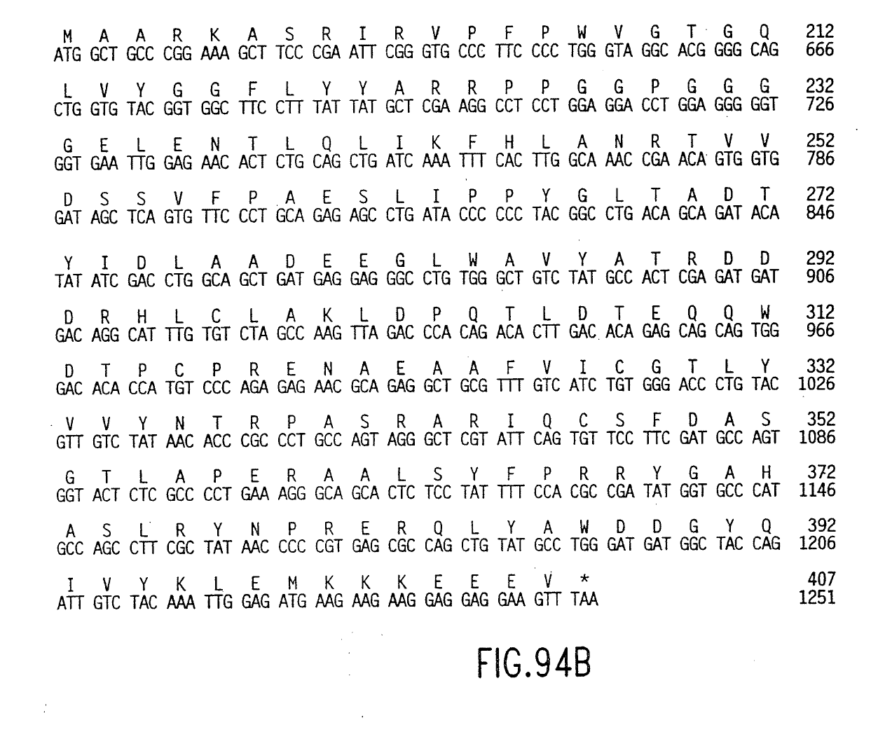

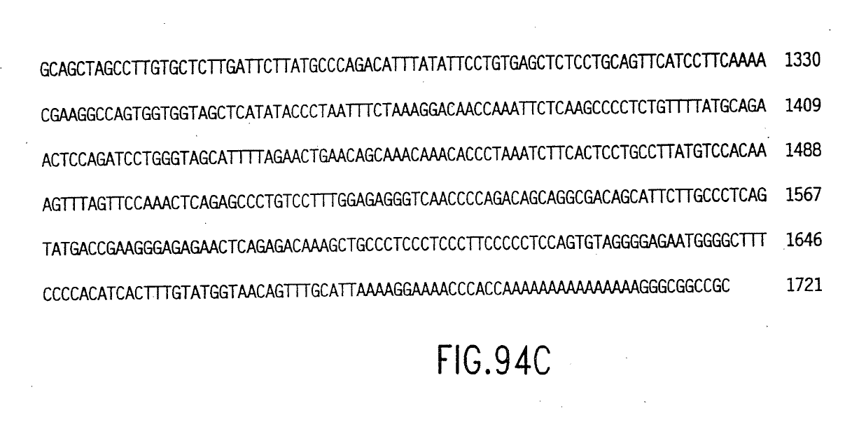

A cDNA encoding mouse TANGO 257 was identified by analyzing the sequences of clones present in a mouse microglia library using a rat TANGO 257 probe. This analysis led to the identification of a clone, Atmua102gb1, encoding full-length mouse TANGO 257. The mouse TANGO 257 cDNA of this clone is 1721 nucleotides long (FIG. 94A-94C; SEQ ID NO:73). The open reading frame of this cDNA, nucleotides 31 to 1248, encodes a 406 amino acid secreted protein (SEQ ID NO:74).

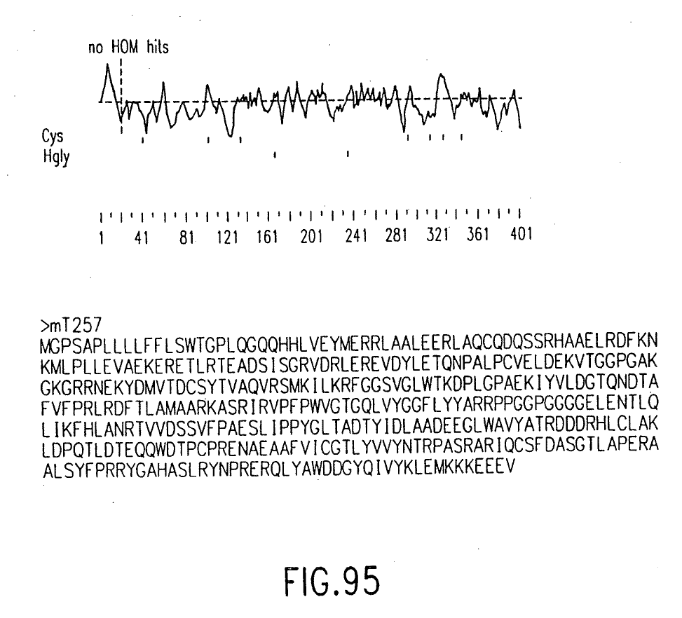

FIG. 95 depicts a hydropathy plot of mouse TANGO 257.

The signal peptide prediction program SIGNALP (Nielsen et al., 1997, Protein Engineering 10:1-6) predicted that mouse TANGO 257 includes a 21 amino acid signal peptide (amino acid 1 to amino acid 21 of SEQ ID NO:74) preceding the mature TANGO 257 protein (corresponding to amino acid 22 to amino acid 406 of SEQ ID NO:74). The molecular weight of mouse TANGO 257 protein without post-translational modifications is 45.8 kDa prior to the cleavage of the signal peptide, 43.6 kDa after cleavage of the signal peptide.

Two N-glycosylation sites are present in mouse TANGO 257. The first has the sequence NDTA and is found at amino acids 177 to 180, and the second has the sequence NRTV and is found at amino acids 248 to 251. A cAMP and cGMP-dependent protein kinase phosphorylation site having the sequence RKAS is found in mouse TANGO 257 at amino acids 196 to 199. Five protein kinase C phosphorylation sites are present in mouse TANGO 257. The first has the sequence SSR (at amino acids 48 to 50), the second has the sequence TLR (at amino acids 75 to 77), the third has the sequence SGR (at amino acids 84 to 86), the fourth has the sequence SMK (at amino acids 144 to 146) and the fifth has the sequence SLR (at amino acids 374 to 376). Five casein kinase II phosphorylation sites are present in mouse TANGO 257. The first has the sequence TEAD (at amino acids 78 to 81), the second has the sequence TQND (at amino acids 175 to 178), the third has the sequence TVVD (at amino acids 250 to 253), the fourth has the sequence TYID (at amino acids 272 to 275), and the fifth has the sequence TRRD (at amino acids 289 to 292). Mouse TANGO 5257 has a tyrosine kinase phosphorylation site having the sequence RLEREVDY at amino acids 89 to 96. Mouse TANGO 257 has four N-myristylation sites. The first has the sequence GGPGAK (at amino acids 115 to 120), the second has the sequence GGSVGL (at amino acids 151 to 157), the third has the sequence GGPGGG (at amino acids 227 to 232), and the fourth has the sequence GAHASL (at amino acids 370 to 375). Mouse TANGO 257 has an amidation site having the sequence KGRR at amino acids 122 to 125.

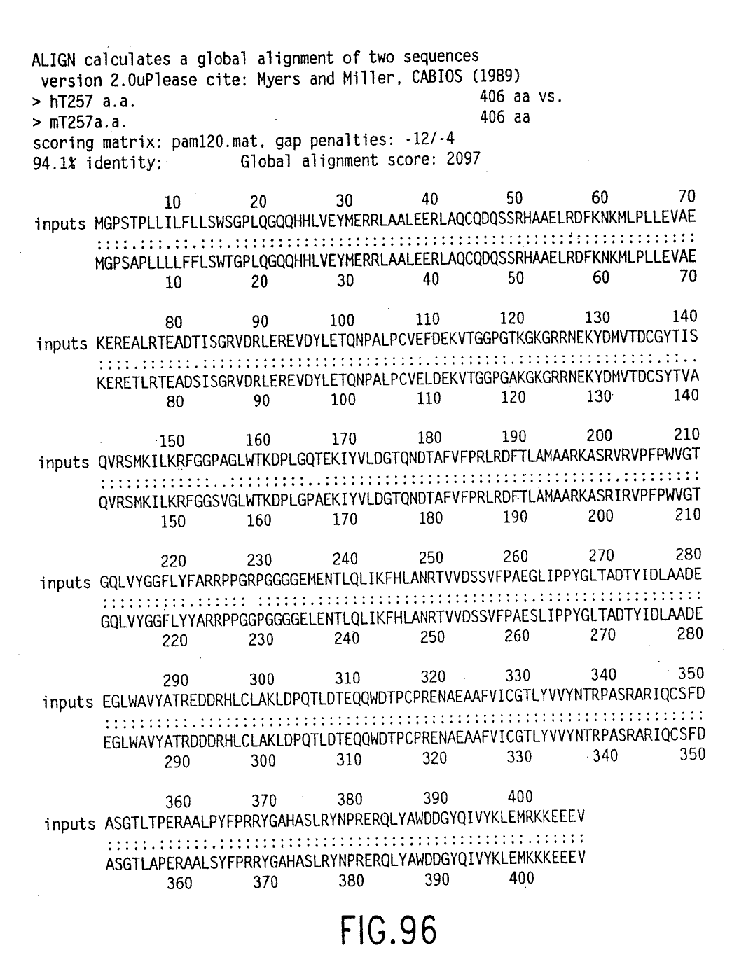

As shown in FIG. 96, human TANGO 257 protein and mouse TANGO 257 protein are 94.1% identical.

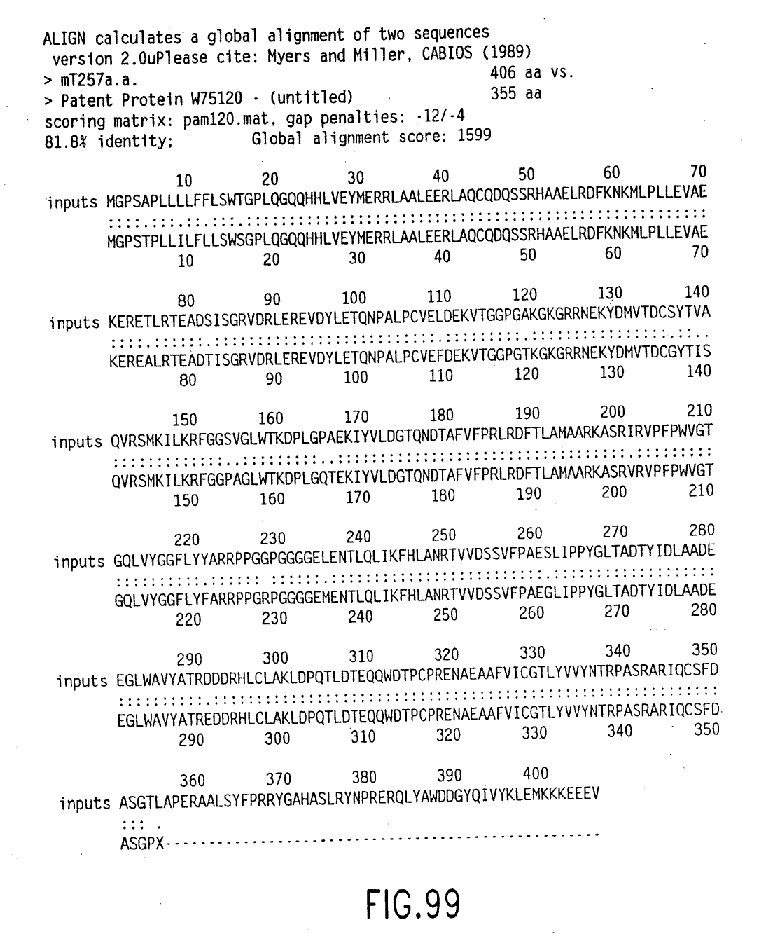

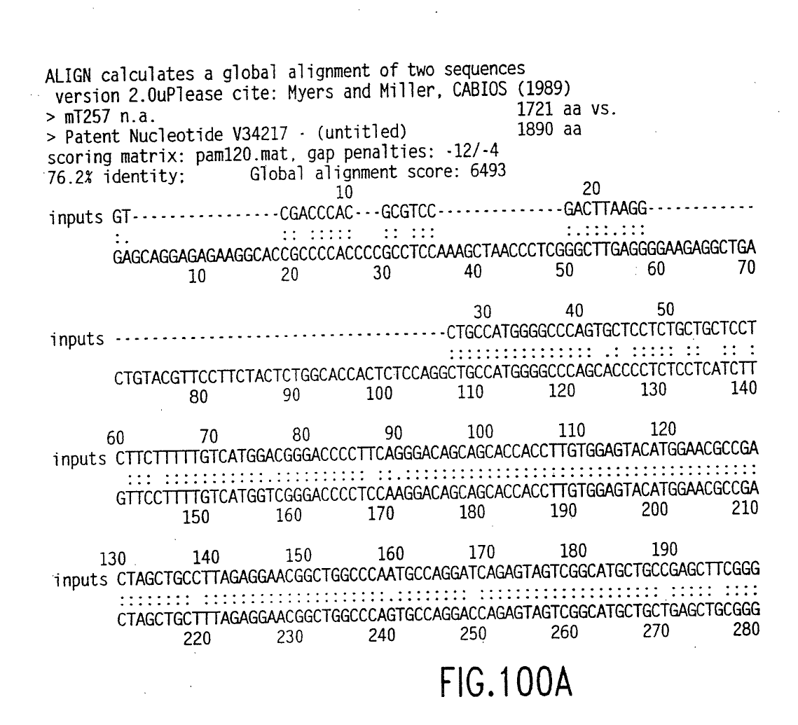

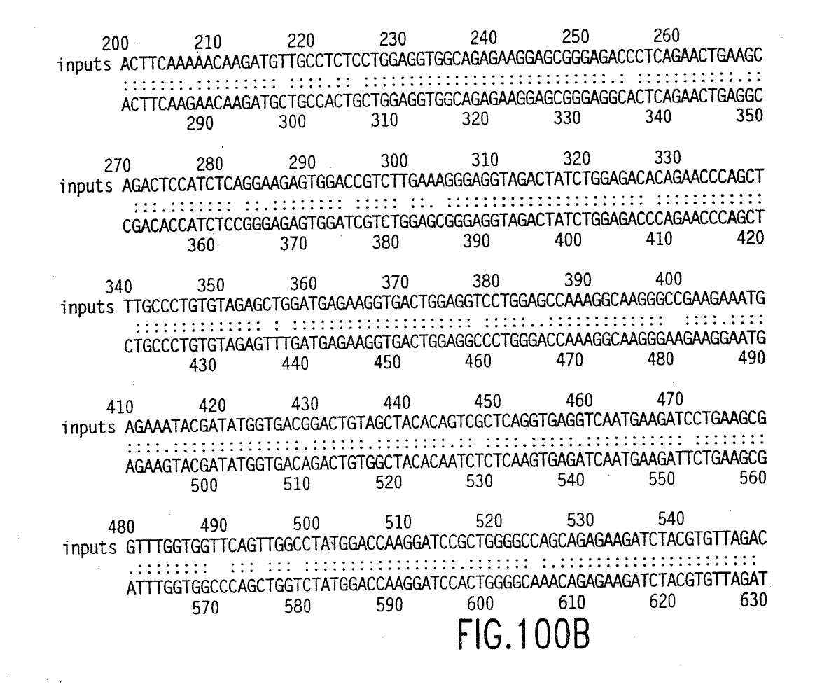

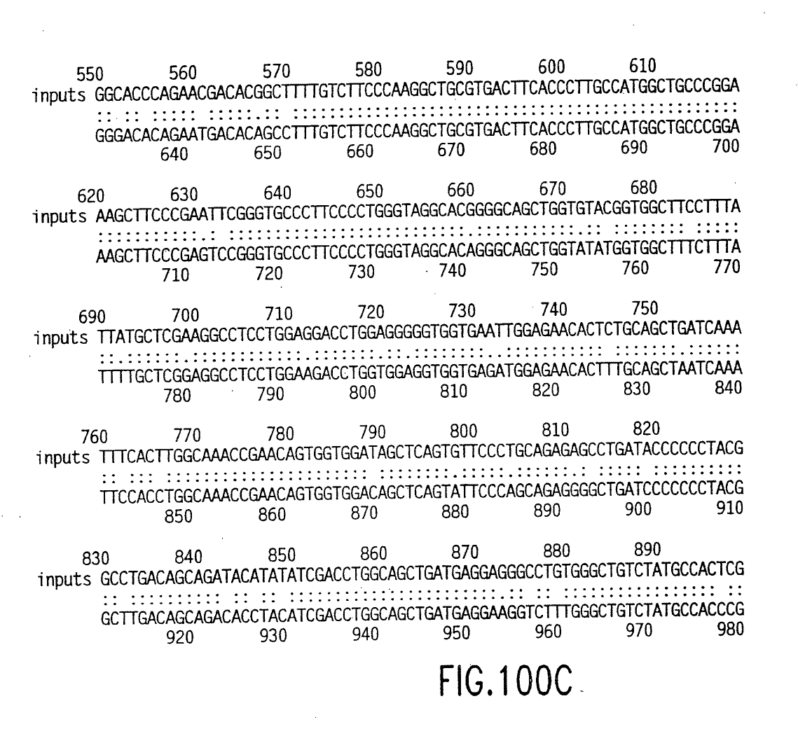

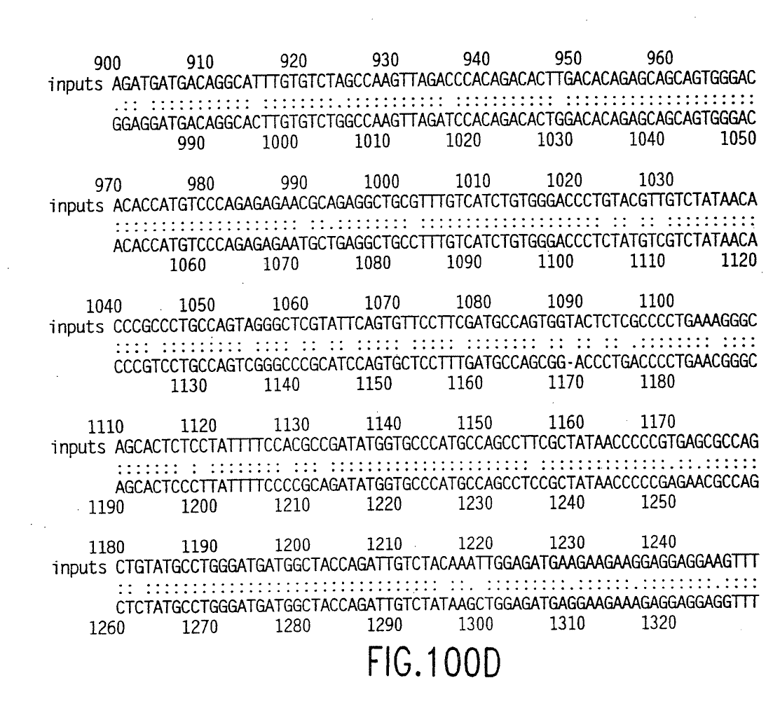

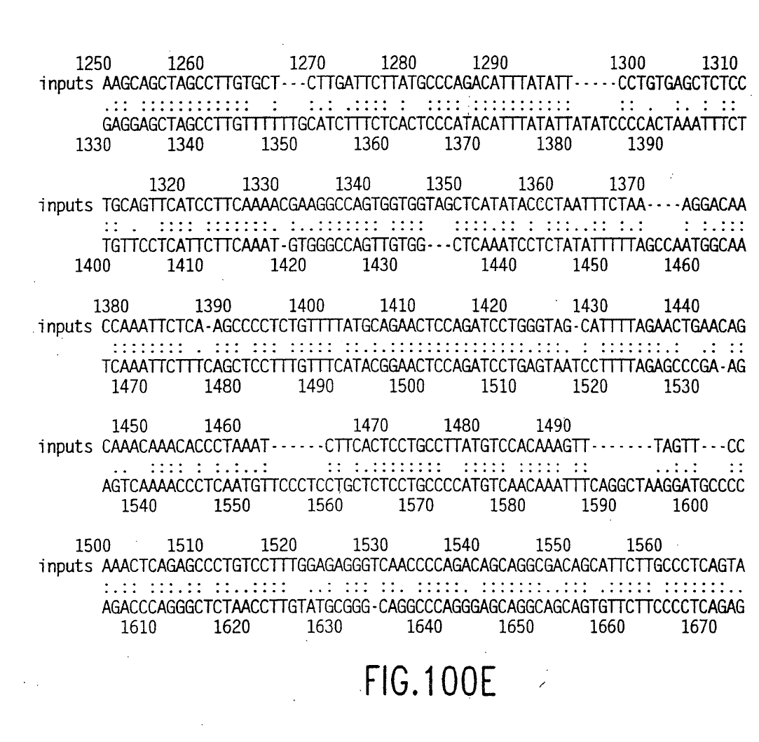

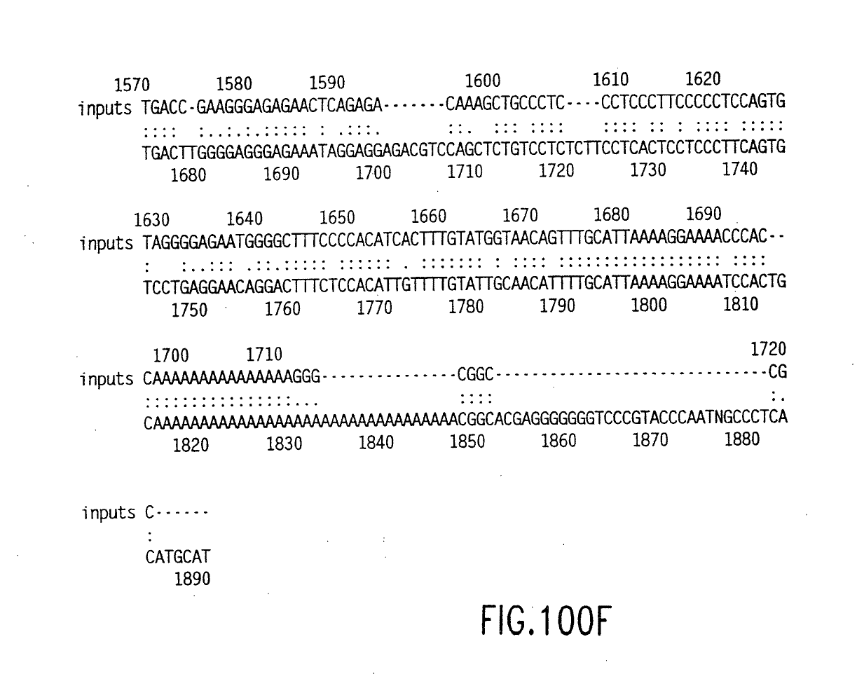

FIG. 99 shows an alignment of mouse TANGO 257 amino acid sequence with the amino acid sequence encoded by gene 64. As shown in the FIGURE, the 253 amino acid gene 64 polypeptide and the 406 amino acid mouse TANGO 257 polypeptide and the 406 amino acid mouse TANGO 257 polypeptide are approximately 82% identical. FIG. 100A-F show an alignment of the nucleotide sequence of gene 64 (PCT publication no. 98/39446) and the nucleotide sequence of mouse TANGO 257. As shown in the FIG. 100A-100F, the two nucleotide sequences are approximately 76% identical.

In situ tissue screening was performed on mouse adult tissues and embryonic tissues (obtained from embryos E13.5 to P1.5) to analyze for the expression of mouse TANGO 257 mRNA. Mouse TANGO 257 expression was detected the following adult tissues: the submandibular gland; the renal papilla region of the kidney; the capsule region of the adrenal gland; and the labyrinth zone of the placenta.

In the case of embryonic expression, mouse TANGO 257 expression was detected in the bones, lungs, intestines, and kidneys. At E13.5, a signal was detected in many tissues including the developing bone structures such as the vertebrae, of the spinal column, jaw, and scapula. At E14.5, the signal pattern was very similar to that detected at E13.5. At 15.5, a signal was detected in all major bone structures, including the skull, basisphenoid bone, upper and lower incisor teeth, vertebral column, sternum, scapula, and femur. A ubiquitous signal was also detected in the lung, kidney, and intestinal tract. At 16.5 and 18.5, the signal is very similar to that detected at E15.5. At P1.5, a signal was still detected in all of the major bone structures and signal detected in the lung, kidney, and intestines has dropped to nearly background levels.

Clone EpTm257, which encodes mouse TANGO 257, was deposited with the American Type Culture Collection (10801 University Boulevard, Manassas, Va. 20110-2209) on Apr. 21, 1999 and assigned Accession Number 207117. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 257 Nucleic Acids, Polypeptides, and Modulators Thereof

As TANGO 257 was originally found in a coronary artery smooth muscle library, TANGO 257 nucleic acids, proteins, and modulators thereof can be used to modulate the proliferation, development, differentiation, and/or function of organs, e.g., heart, tissues and cells that form blood vessels and coronary tissue, e.g., cells of the coronary connective tissue, e.g., coronary smooth muscle cells and/or endothelial cells of blood vessels. TANGO 257 nucleic acids, proteins, and modulators thereof can also be used to modulate symptoms associated with abnormal coronary function, e.g., heart diseases and disorders such as atherosclerosis, coronary artery disease and plaque formation.

In light of TANGO 257's homology to the extracellular molecule olfactomedin, TANGO 257 nucleic acids, proteins and modulators thereof can be utilized to modulate development, differentiation, proliferation and/or activity of neuronal cells, e.g., olfactory neurons and to modulate neuronal activities involving maintenance, growth and/or differentiation of chemosensory cilia, modulate cell-cell interactions and cell-ECM interactions, e.g., neuronal (such as olfactory) cell-ECM interactions. TANGO 257 nucleic acids, proteins and modulations thereof can also be used to modulate symptoms associated with abnormal processes involving such cells and/or activities, for example neuronal function, e.g., neurological disorders, neurodegenerative disorders, neuromuscular disorders, cognitive disorders, personality disorders, and motor disorders, and chemosensory disorders, such as olfactory-related disorders.

TANGO 257 exhibits homology to a gene referred to as “gene 64” (PCT Publication No. WO 98/39446), which is expressed primarily in fetal lung tissue. In light of this, TANGO 257 nucleic acids, proteins and modulators thereof can also be used to modulate development, differentiation, proliferation and/or activity of pulmonary system cells, e.g., lung cell types, and to modulate a symptom associated with disorders of pulmonary development, differentiation and/or activity, e.g., cystic fibrosis. TANGO 257 nucleic acids, proteins and modulators thereof can also be used to modulate symptoms associated with abnormal pulmonary development or function, such as lung diseases or disorders associated with abnormal pulmonary development or function, e.g., cystic fibrosis. TANGO 257 nucleic acids, polypeptides, or modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis, Goodpasture's syndrome, diopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

TANGO 257 nucleic acids, proteins and modulators thereof can also be used to modulate cell proliferation, e.g., abnormal cell proliferation. Such modulation may, for example, be via modulation of one or more elements involved in signal transduction cascades.

TANGO 257 nucleic acids, proteins and modulators thereof can also be utilized to modulate the development, differentiation, maturation, proliferation and/or activity of bone cells such as osteocytes, and to treat bone associated diseases or disorders. Examples of bone diseases and disorders include bone injury due to for example, trauma (e.g. bone breakage, cartilage tearing), degeneration (e.g., osteoporosis), degeneration of joints, e.g., arthritis, e.g., osteoarthritis, and bone wearing. Further, TANGO 257 nucleic acids, proteins and modulators thereof can be utilized to modulate or regulate the development of bone structures such as the skull, the basisphenoid bone, the upper and lower incisor teeth, the vertebral column, the sternum, the scapula, and the femur during embryogenesis.

TANGO 257 nucleic acids, proteins and modulators thereof can, in addition to the above, be utilized to regulate or modulate development and/or differentiation of processes involved in microglial, liver, kidney, and skeletal muscle formation and activity, as well as in ameliorating a symptom associated with a disorder of such cell types, tissues and organs.

TANGO 257 nucleic acids, polypeptides, or modulators thereof can also be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy), acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma). TANGO 257 polypeptides, nucleic acids, or modulators thereof can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

Further, TANGO 257 expression can be utilized as a marker (e.g. an in situ marker) for specific tissues (i.e., bone structures) and/or cells (i.e., osteocytes) in which TANGO 257 is expressed. TANGO 257 nucleic acids can also be used for chromosomal mapping.

### Human INTERCEPT 258

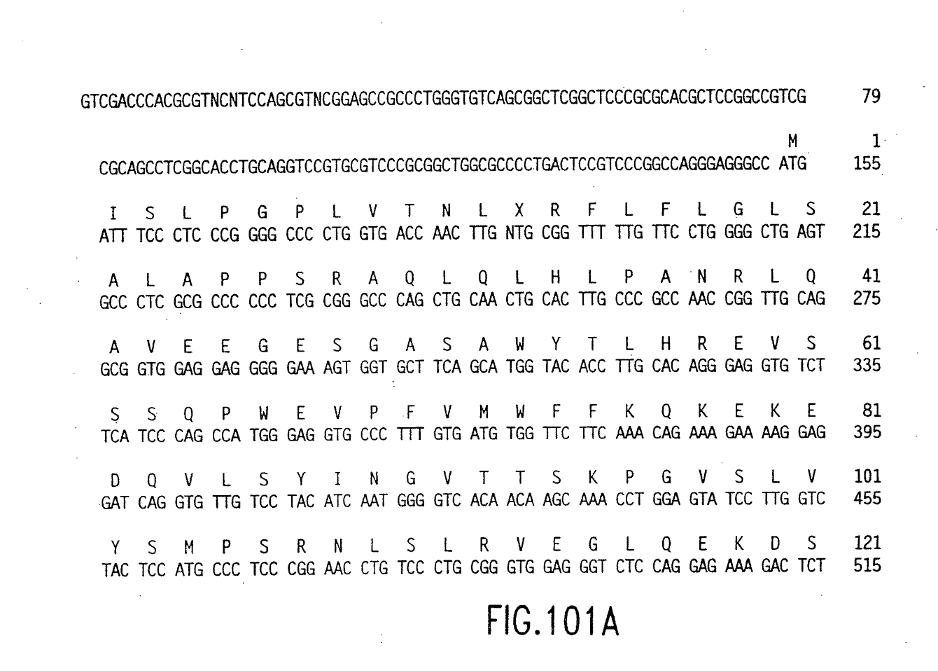

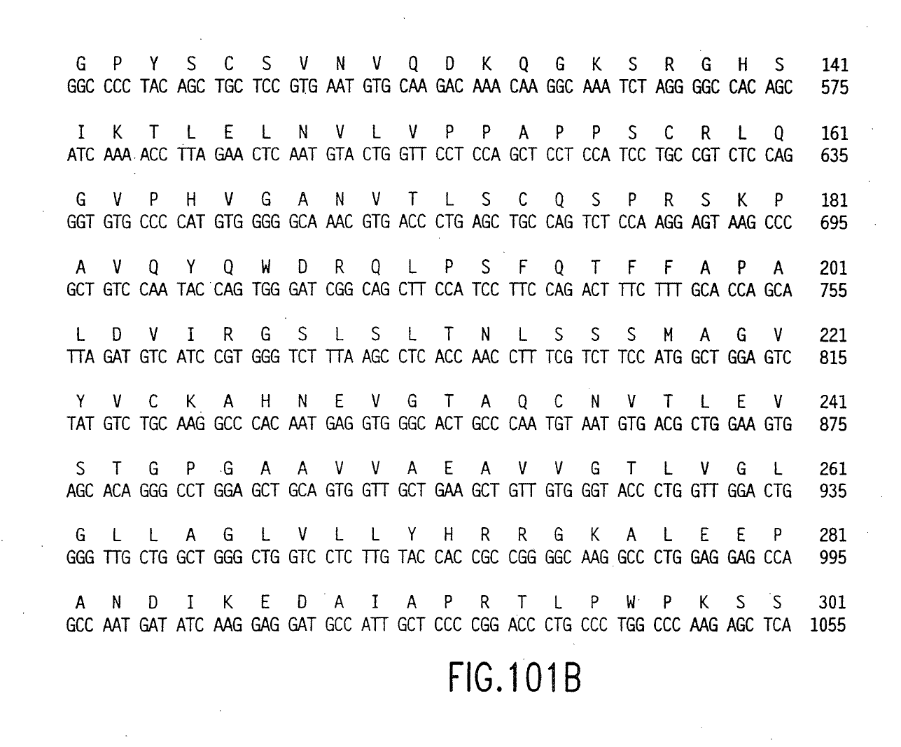

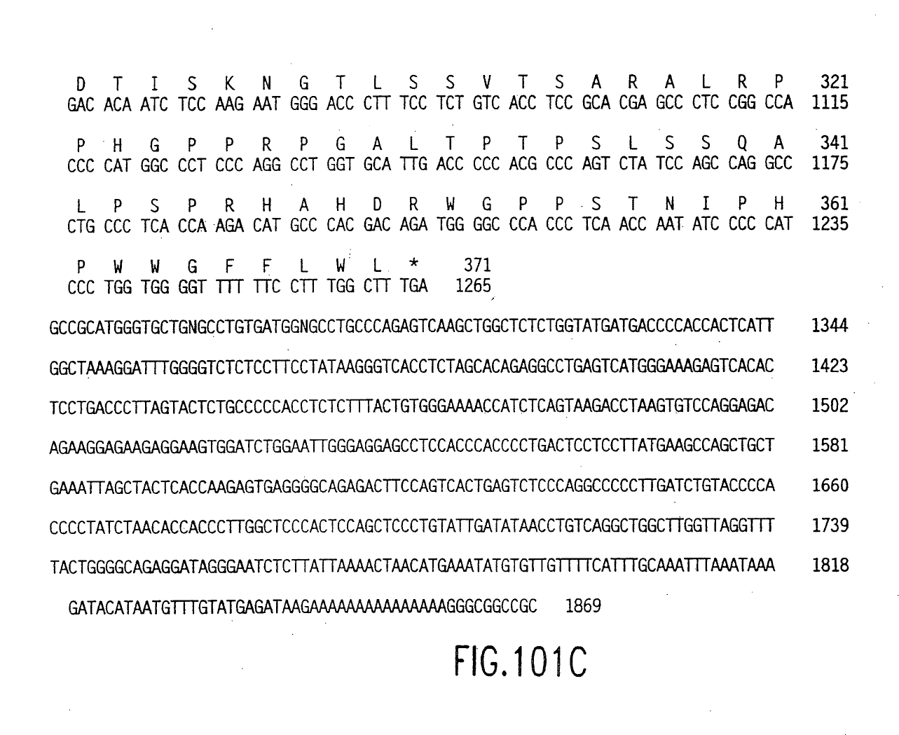

A cDNA encoding human INTERCEPT 258 was identified by analyzing the sequences of clones present in a human mixed lymphocyte reaction library for sequences that encode secreted proteins. This analysis led to the identification of a clone, Athlxtce, encoding full-length human INTERCEPT 258. The human INTERCEPT 258 cDNA of this clone is 1869 nucleotides long (FIG. 101A-101C; SEQ ID NO:75). The open reading frame of this cDNA (nucleotides 153 to 1262 of SEQ ID NO:75) encodes a 370 amino acid transmembrane protein (SEQ ID NO:76).

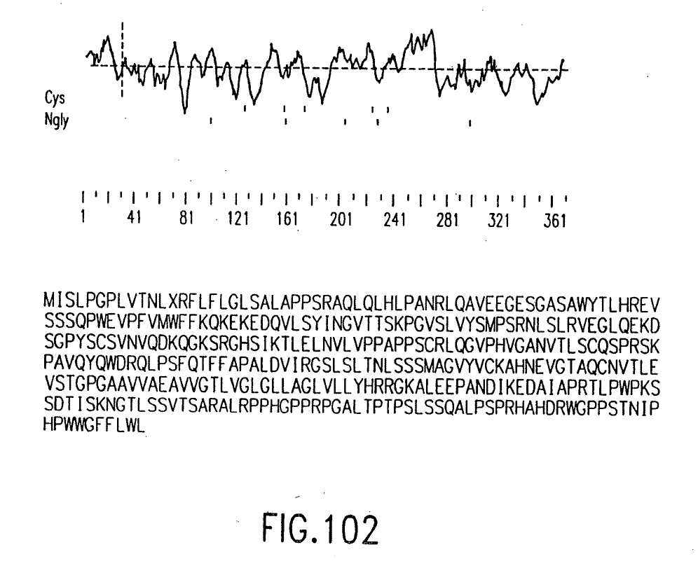

FIG. 102 depicts a hydropathy plot of human INTERCEPT 258. The dashed vertical line separates the signal sequence (amino acids 1 to 29 of SEQ ID NO:76) on the left from the mature protein (amino acids 30 to 370 of SEQ ID NO:76) on the right.

The signal peptide prediction program SIGNALP (Nielsen et al., 1997, Protein Engineering 10:1-6) predicted that human INTERCEPT 258 includes a 29 amino acid signal peptide (amino acid 1 to amino acid 29 of SEQ ID NO:76) preceding the mature INTERCEPT 258 protein (corresponding to amino acid 30 to amino acid 370 of SEQ ID NO:76). The molecular weight of human INTERCEPT 258 protein without post-translational modifications is 40.0 kDa prior to the cleavage of the signal peptide, 37.0 kDa after cleavage of the signal peptide.

Human INTERCEPT 258 contains a hydrophobic transmembrane domain at amino acids amino acids 207 to 224 and amino acids 247 to 271 of SEQ ID NO:76. Human INTERCEPT 258 also contains two Ig domains, one at amino acids 49 to 128 of SEQ ID NO:76 and a second at amino acids 167 to 226 of SEQ ID NO:76.

Five N-glycosylation sites are present in human INTERCEPT 258. The first has sequence NLSL and is found at amino acids 108 to 111, the second has the sequence NUTL and is found at amino acids 169 to 172; the third is has the sequence NLSS and is found at amino acids 213 to 216, the fourth has the sequence NUTL and is found at amino acids, 236 to 239, and the fifth has the sequence NGTL and is found at amino acids 307 to 310. Seven protein kinase C phosphorylation sites are present in human INTERCEPT 258. The first has the sequence TSK and is found at amino acids 93 to 95, the second has the sequence SLR and is found at amino acids 110 to 112, the third has the sequences SIK and is found at amino acids 141 to 143, the fourth has the sequence SCR and is found at amino acids 157 to 159, the fifth has the sequence SPR and is found at amino acids 176 to 179, the sixth has the sequence SAR and is found at amino acids 315 to 317, and the seventh has the sequence SPR and is found at amino acids 344 to 346. The human INTERCEPT 258 protein has seven N-myristoylation sites. The first has the sequence GUTTSK and is found at amino acids 90 to 95, the second has the sequence GANVTL and is found at amino acids 167 to 172, the third has the sequence GVYVCK and is found at amino acids 220 to 225, the fourth has the sequence GTAQCN and is found at amino acids 231 to 236, the fifth has the sequence GTLVGL and is found at amino acids 256 to 261, the sixth has the sequence GLLAGL and is found at amino acids 262 to 267, and the seventh has the sequence GTLSSU and is found at acids 308 to 313.

The human INTERCEPT 258 gene was mapped to human chromosome 11 using Genebridge 4 Human Radiation hybrid mapping panel with GGAGTATCCTTGGTCTACTCC as the forward primer and GAAAGTCTGGAAGGATGGAAGCT as the reverse primer.

Human multi-tissue dot blot analysis of human INTERCEPT 258 expression demonstrates strongest expression in lung, fetal lung, placenta, thyroid gland and mammary gland. Moderate expression is observed in heart, aorta, kidney, small intestine, fetal heart, fetal kidney, fetal spleen, uterus, and stomach. Weak expression is observed in whole brain, amygdala, caudate nucleus, cerebellum, cerebral cortex frontal lobe, hippocampus, medulla oblongata, occipital lobe, putamen, substantia nigra, temporal lobe, thalamus, acumens, spinal cord, skeletal muscle, colon, bladder, prostate, ovary, pancreas, pituitary gland, adrenal gland, salivary gland, liver, spleen, thymus, lymph node, bone marrow, appendix, trachea, fetal brain, fetal liver, and fetal thymus.

A human cancer cell line Northern blot analysis showed a roughly 2.0 kb INTERCEPT 258 band only in the lane containing cell line Chronic Myelogenous Leukemia (K-562). The cancerous cell lines in which INTERCEPT 258 was not expressed include promyeocytic leukemia, Hela, lymphoblastic leukemia, Burkitt's lyniphoma Raji, colorectal adenocarcinoma, lung carcinoma and melanoma.

INTERCEPT 258 exhibits homology to a human A33 antigen. A33 antigen is a transmembrane glycoprotein and a member of the immunoglobulin superfamily that may represent a cancer cell marker (Heath et al., 1997, Proc. Natl. Acad. Sci. USA 94:469-474).

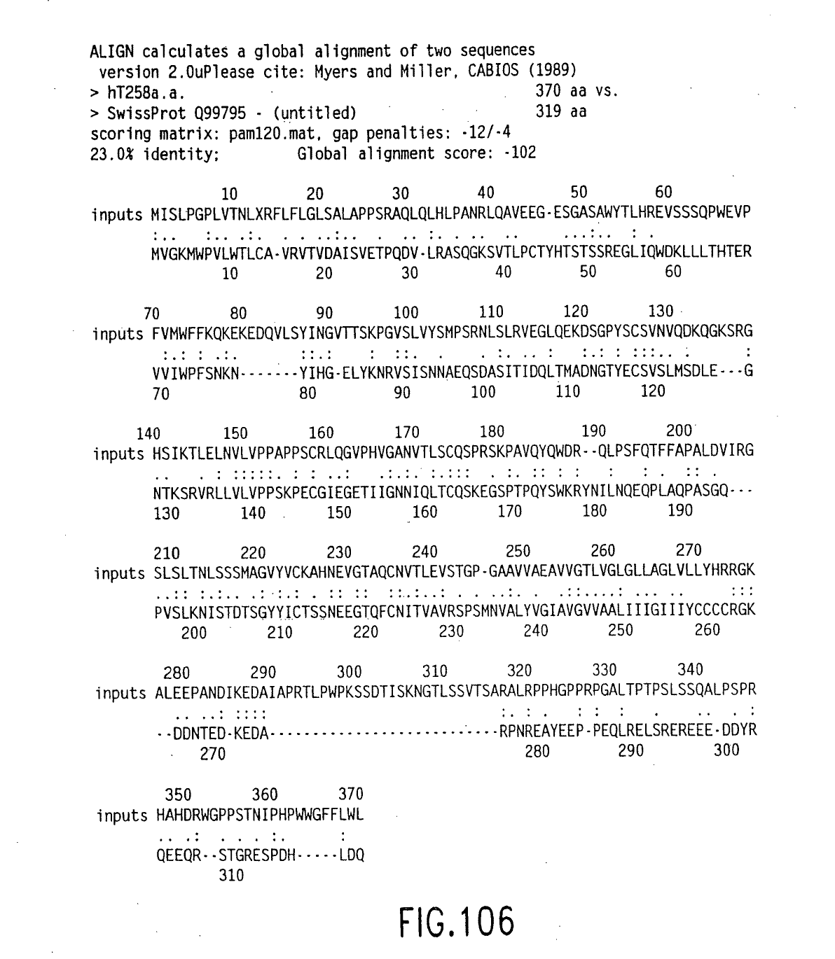

FIG. 106 shows an alignment of the human INTERCEPT 258 amino acid sequence with the human A33 amino acid sequence. The alignment shows that there is a 23.0% overall amino acid sequence identity between human INTERCEPT 258 and A33.

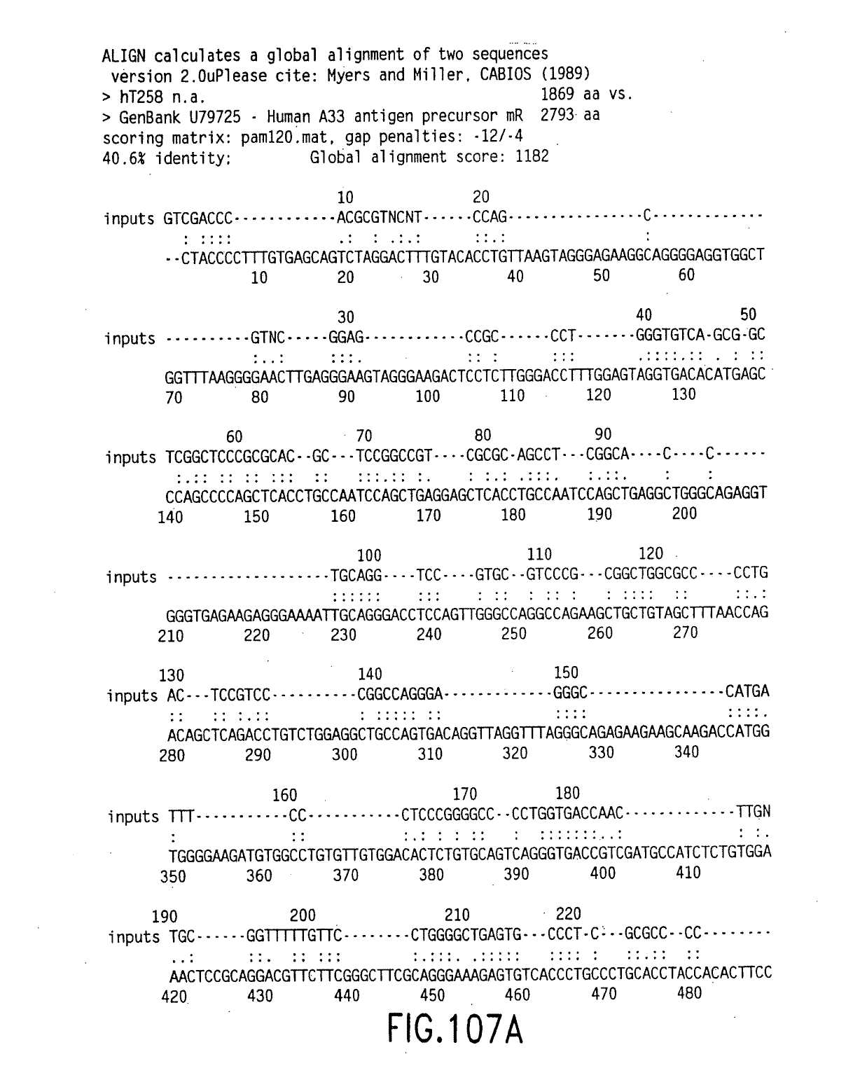

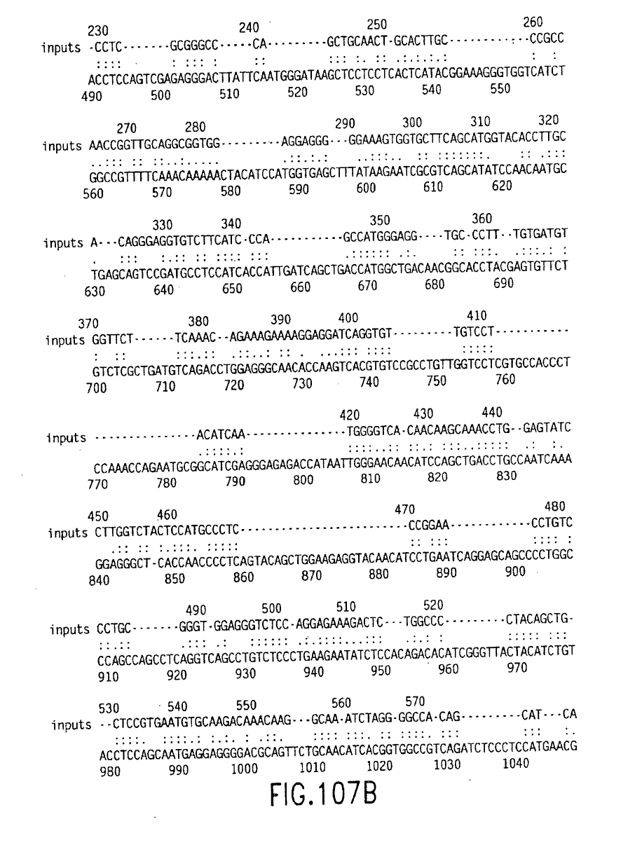

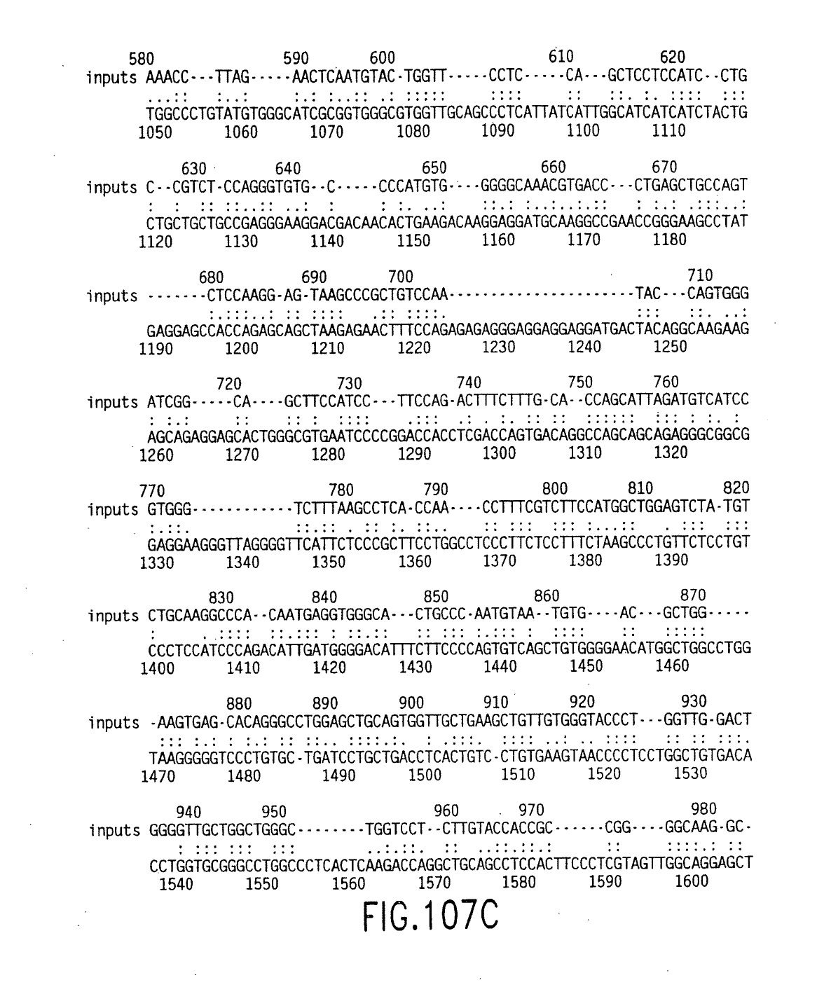

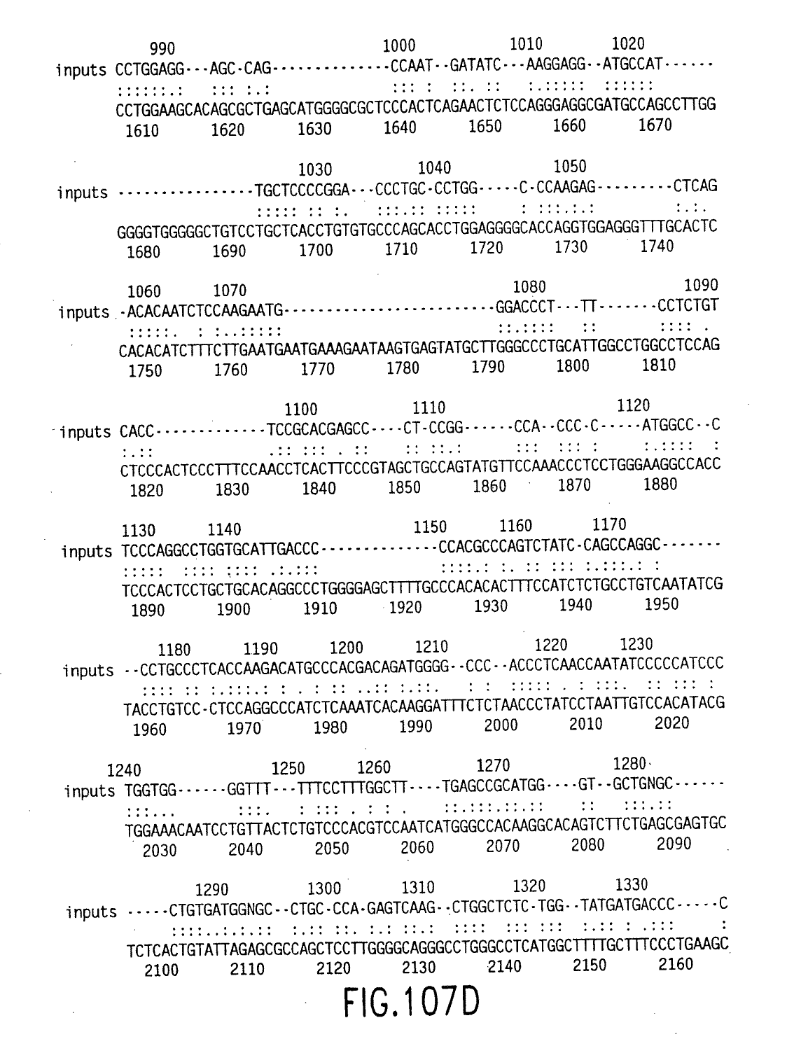

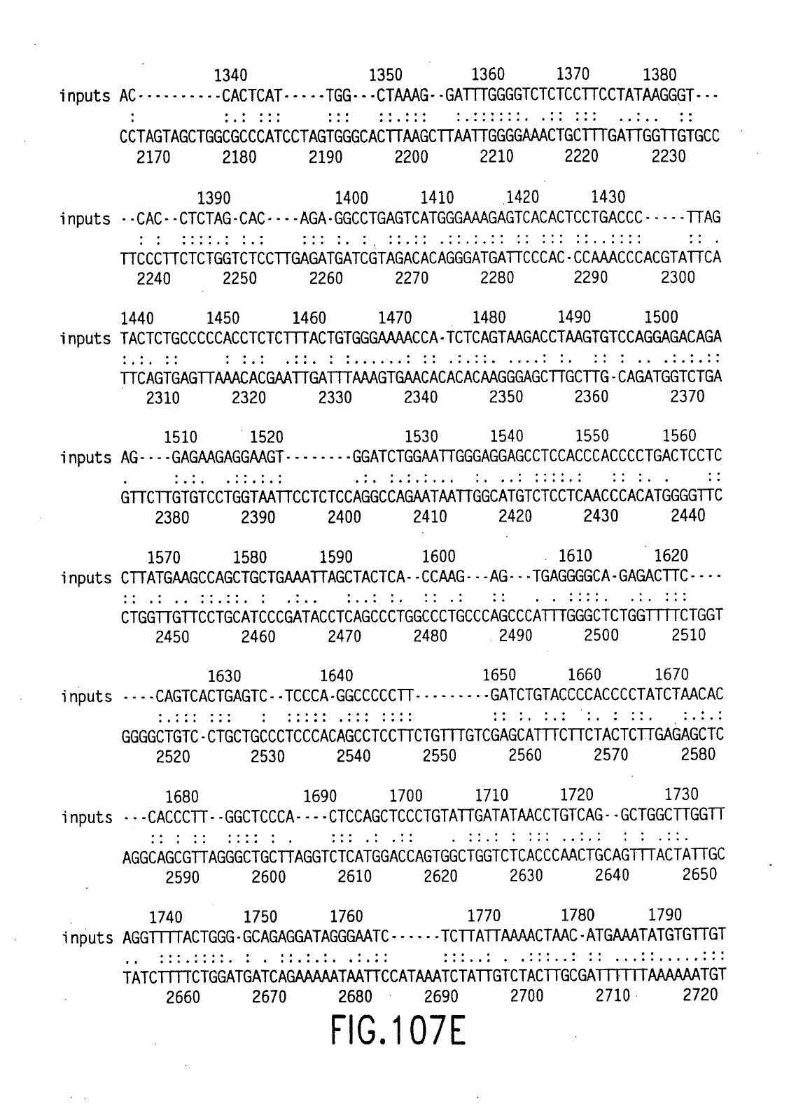

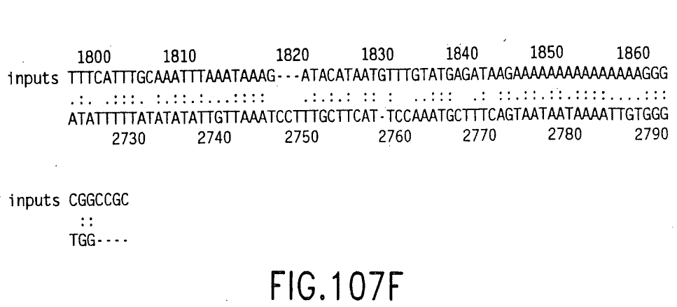

FIG. 107A-107F show an alignment of the human INTERCEPT 258 nucleotide sequence with that of human A33 nucleotide sequence. The alignment shows that there is a 40.6% identity between the two sequences.

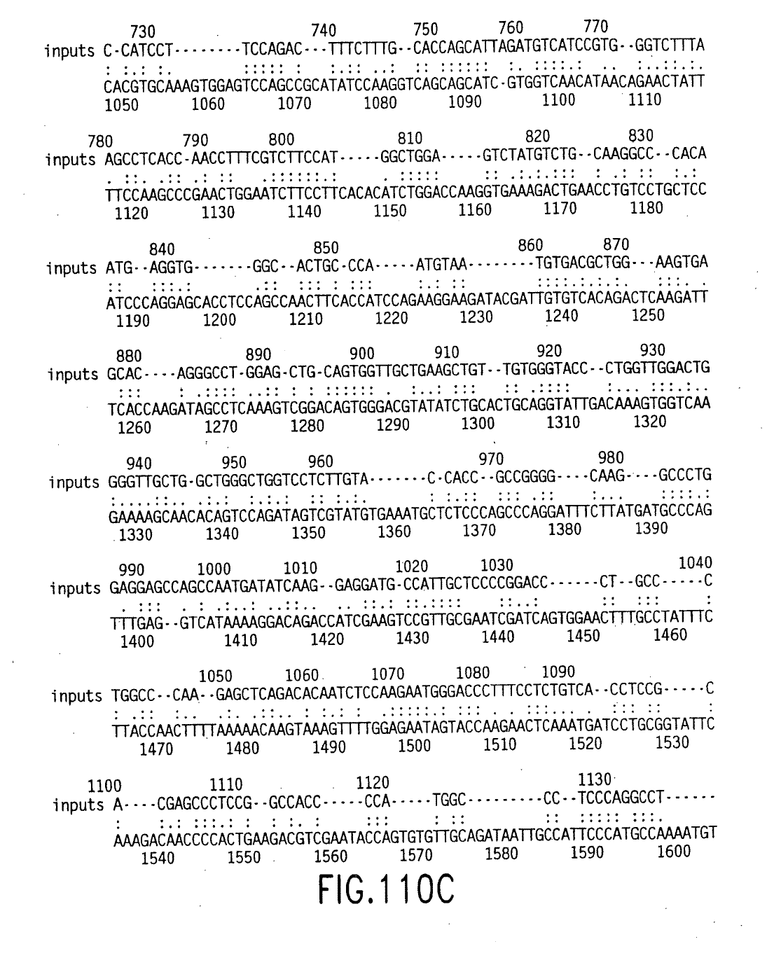

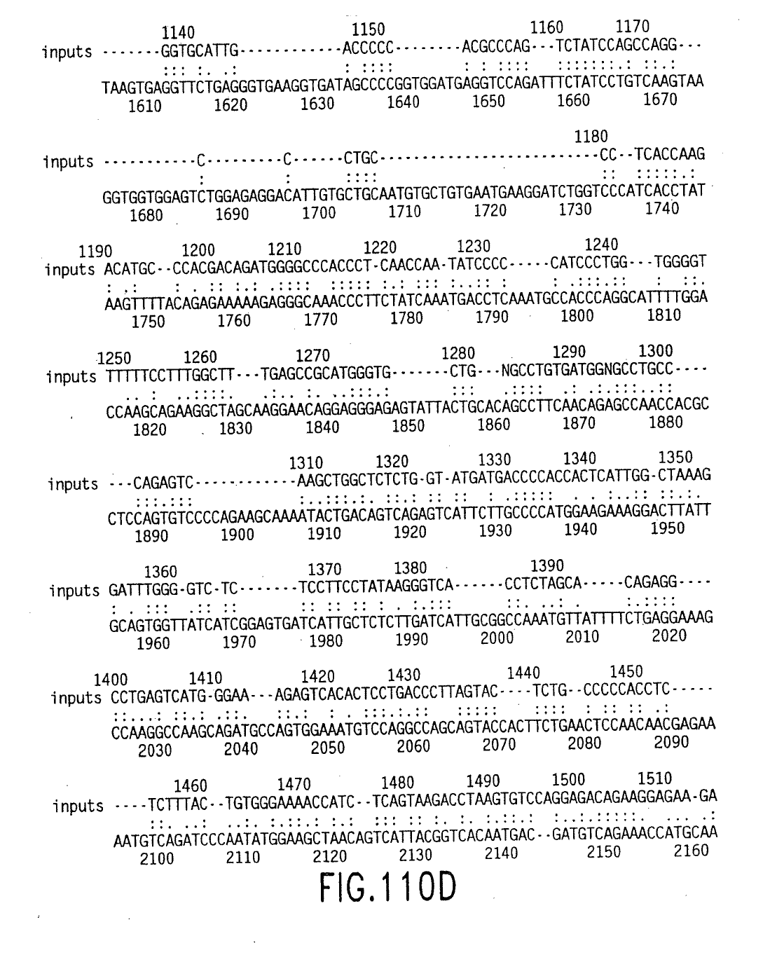

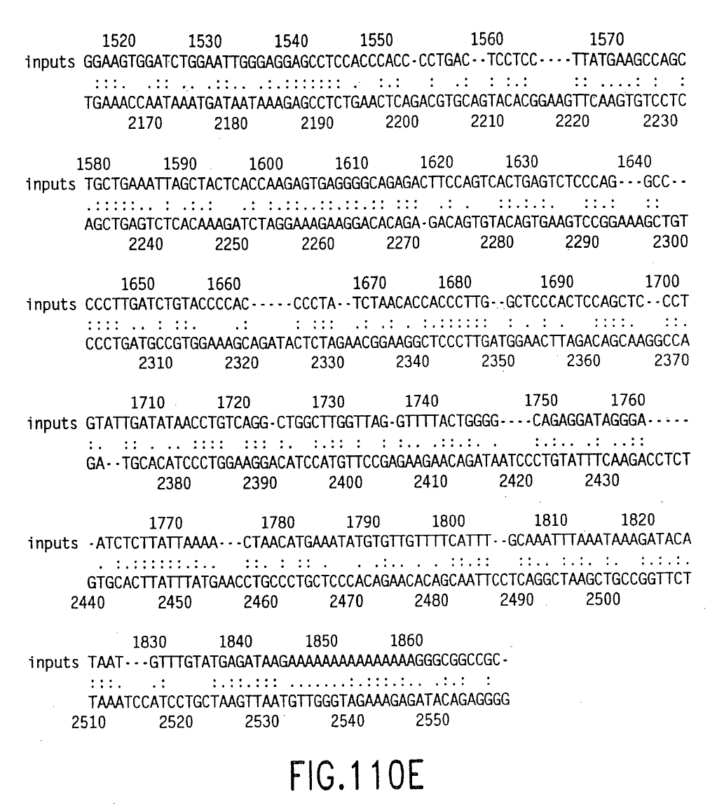

Human INTERCEPT 258 nucleotide sequence exhibits homology to human PECAM-1 nucleotide sequence. FIG. 110A-110E show that there is an overall 40.5% identity between the two nucleotide sequences. Human INTERCEPT 258 amino acid sequence and human PECAM-1 amino acid sequence share less than 18% identity. PECAM-1 (platelet endothelial cell adhesion molecule-1) is an integrin expressed on endothelial cells.

Clone EpT258, which encodes human INTERCEPT 258, was deposited with the American Type Culture Collection (10801 University Boulevard, Manassas, Va. 20110-2209) on Apr. 21, 1999 and assigned Accession Number 207222. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Mouse INTERCEPT 258

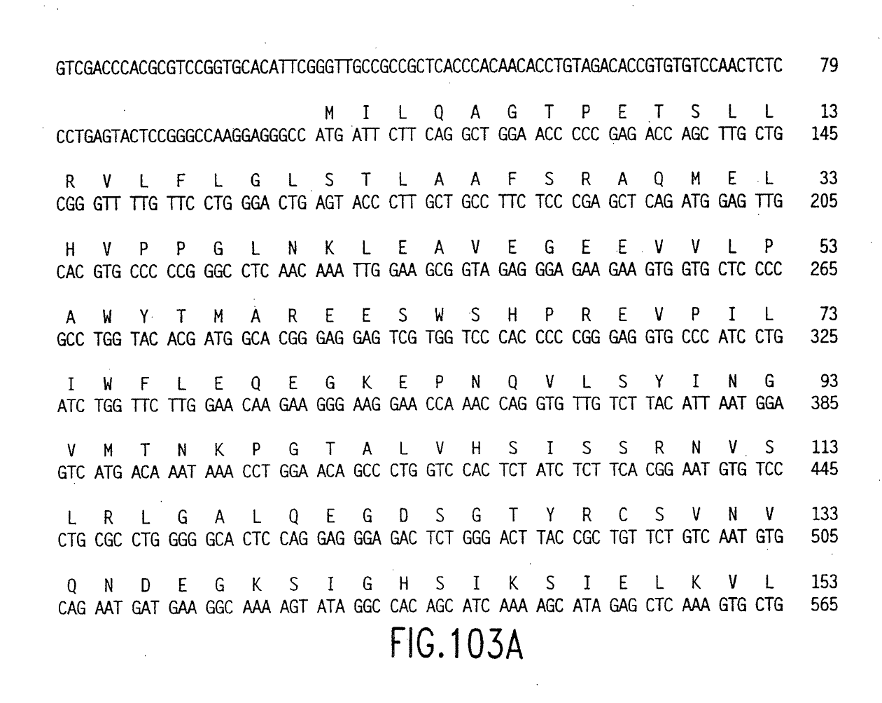

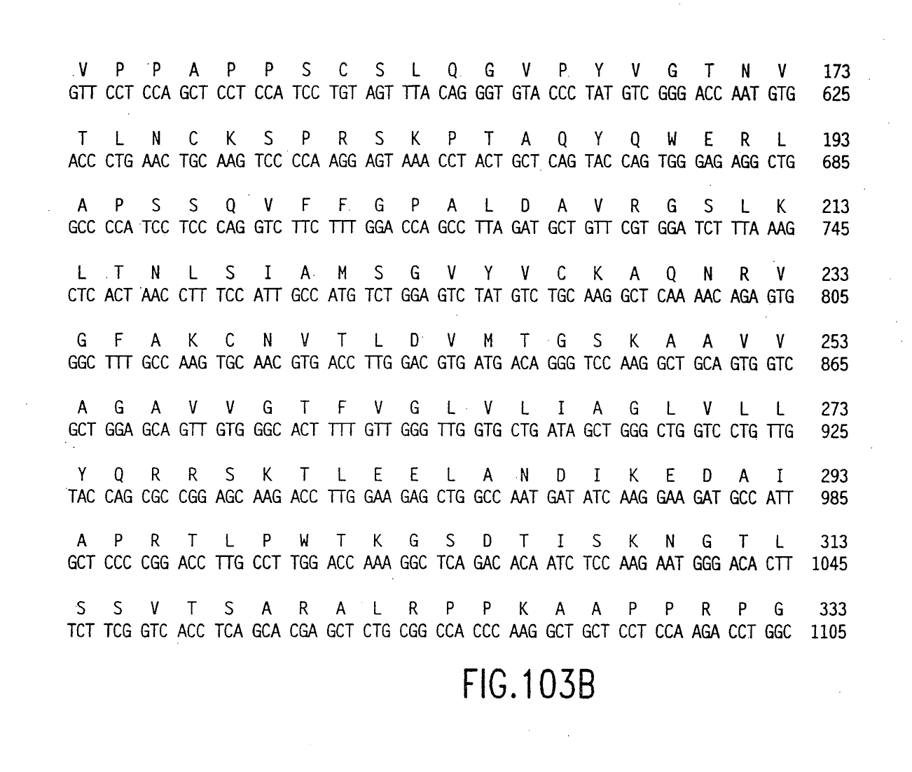

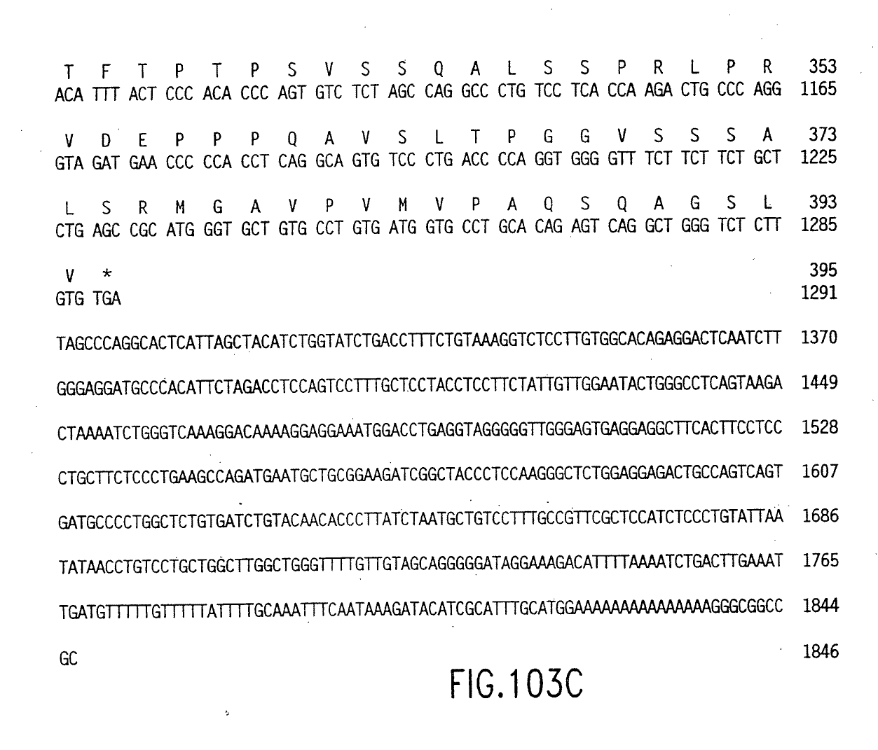

A cDNA encoding mouse INTERCEPT 258 was identified by analyzing the sequences of clones present in a mouse megakaryocyte library for sequences that encode secreted proteins. This analysis led to the identification of a clone, Athmea17c8, encoding full-length mouse INTERCEPT 258. The mouse INTERCEPT 258 cDNA of this clone is 1846 nucleotides long (FIG. 103A-103C; SEQ ID NO:77). The open reading frame of this cDNA (nucleotides 107 to 1288 of SEQ ID NO:77) encodes a 394 amino acid transmembrane protein (SEQ ID NO:78).

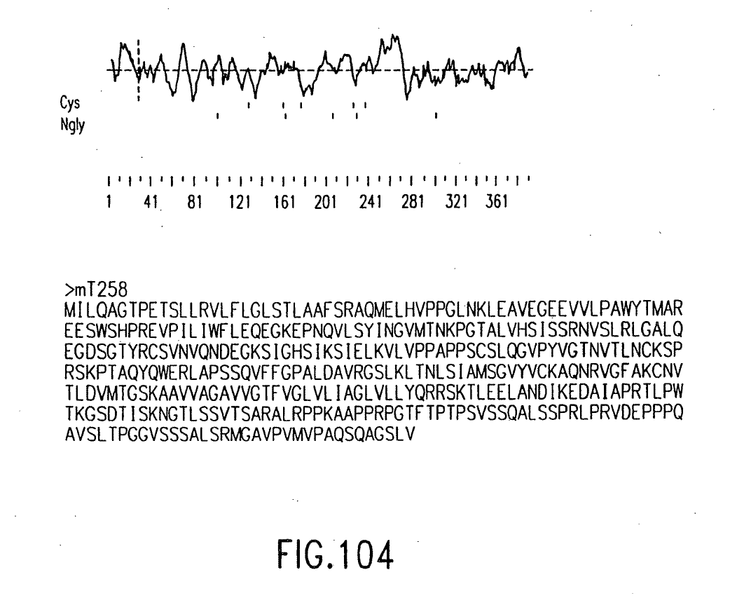

FIG. 104 depicts a hydropathy plot for mouse INTERCEPT 258.

The signal peptide prediction program SIGNALP (Nielsen et al., 1997, Protein Engineering 10:1-6) predicted that mouse INTERCEPT 258 includes a 29 amino acid signal peptide (amino acid 1 to amino acid 29 of SEQ ID NO:78) preceding the mature INTERCEPT 258 protein (corresponding to amino acid 30 to amino acid 394 of SEQ ID NO:78). The molecular weight INTERCEPT 258 without post-translational modifications is 41.8 kDa prior to the cleavage of the signal peptide, 38.90 kDa after cleavage of the signal peptide.

Mouse INTERCEPT 258 contains a hydrophobic transmembrane domain at amino acids 250 to 274 of SEQ ID NO:78. Mouse INTERCEPT 258 also contains an Ig domain at amino acids 170 to 229 of SEQ ID NO:78.

Five N-glycosylation sites are present in mouse INTERCEPT 258. The first has sequence NVSL and is found at amino acids 111 to 114, the second has the sequence NVTL and is found at amino acids 172 to 175, the third has the sequence NLSI and is found at amino acids 216 to 219, the fourth has the sequence NVTL and is found at amino acids, 239 to 242, and the fifth has the sequence NGTL and is found at amino acids 310 to 313. Nine protein kinase C phosphorylation sites are present in mouse INTERCEPT 258. the first has the sequence TNK and is found at amino acids 96 to 98, the second has the sequence SSR and is found at amino acids 108 to 110, the third has the sequence SLR and is found at amino acids 113 to 115, the fourth has the sequence TYR and is found at amino acids 126 to 128, the fifth has the sequence SIK and is found at amino acids 144 to 146, the sixth has the sequence SPR and is found at amino acids 179 to 181, the seventh has the sequence SLK and is found at amino acids 211 and 213, the eighth has the sequence SAR and is found at amino acids 318 to 320, and the ninth has the sequence SPR and is found at amino acids 348 to 350. The mouse INTERCEPT 258 contains a casein kinase II phosphorylation site having the sequence TLEE, found at amino acids 280 to 283. The mouse INTERCEPT 258 protein has nine N-myristoylation sites. The first has the sequence GTPETS and is found at amino acids 6 to 11, the second has the sequence GVMTNK and is found at amino acids 125 to 130, the third has the sequence GTYRCS and is found at amino acids 125 to 130, the fourth has the sequence GTNVTL and is found at amino acids 170 to 175, the fifth has the sequence GVYVCK and is found at amino acids 223 to 228, the sixth has the sequence GSKAAV and is found at amino acids 247 to 252, the seventh has the sequence GAVVGT and is found at amino acids 255 to 260, the eighth has sequence GTLSSV and is found at amino acids 311 to 316, and the ninth has the sequence GGVSSS and is found at amino acids 367 to 372.

An in situ expression analysis of INTERCEPT 258 was performed as summarized herein. Mouse INTERCEPT 258 expression during embryogenesis (E73.5 to P1.5 were examined) was observed throughout the animal in a punctate pattern. This pattern is very similar to that seen with the molecule PECAM-1, but at a lower intensity. PECAM-1 is an integrin expressed on endothelial cells. In addition, lung and brown fat exhibited a much higher signal in a more ubiquitous pattern in all embryonic stages examined. Heart and kidney also have a higher expression, but to a lesser degree. Adult mouse INTERCEPT 258 expression was seen in many tissues, often in a multifocal, punctate pattern suggestive of vessels. Expression was also predominant in many highly vascularized tissues such as ovary (especially the septol region), kidney and adrenal cortex.

In general, both embryonic and adult expression patterns were suggestive of endothelial cells being a component in the expression patters observed. In summary, tissues in which INTERCEPT 258 expression was observed were as follows: brain, eye, harderian gland, submanibular gland, bladder, brown fat, stomach, heart, kidney, adrenal gland, colon, liver, thymus, lymph node, spleen, spinal cord, ovary, testes and placenta.

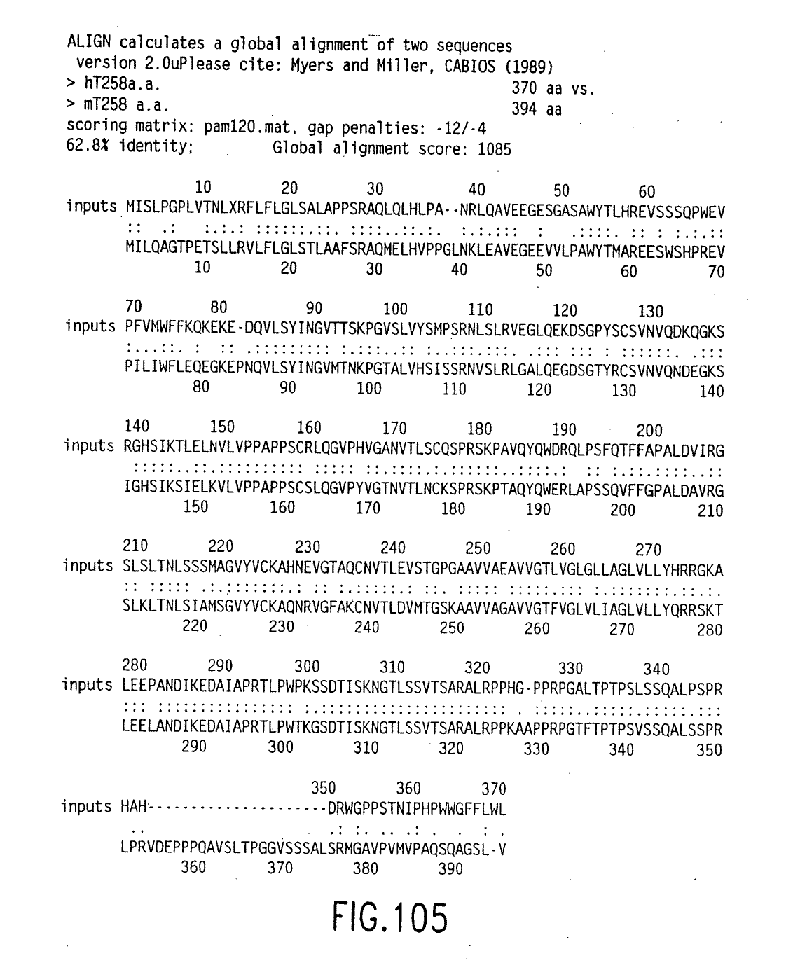

As shown in FIG. 105, human INTERCEPT 258 protein and mouse INTERCEPT 258 protein are 62.8% identical.

Mouse INTERCEPT 258 exhibits homology to a human A33 antigen.

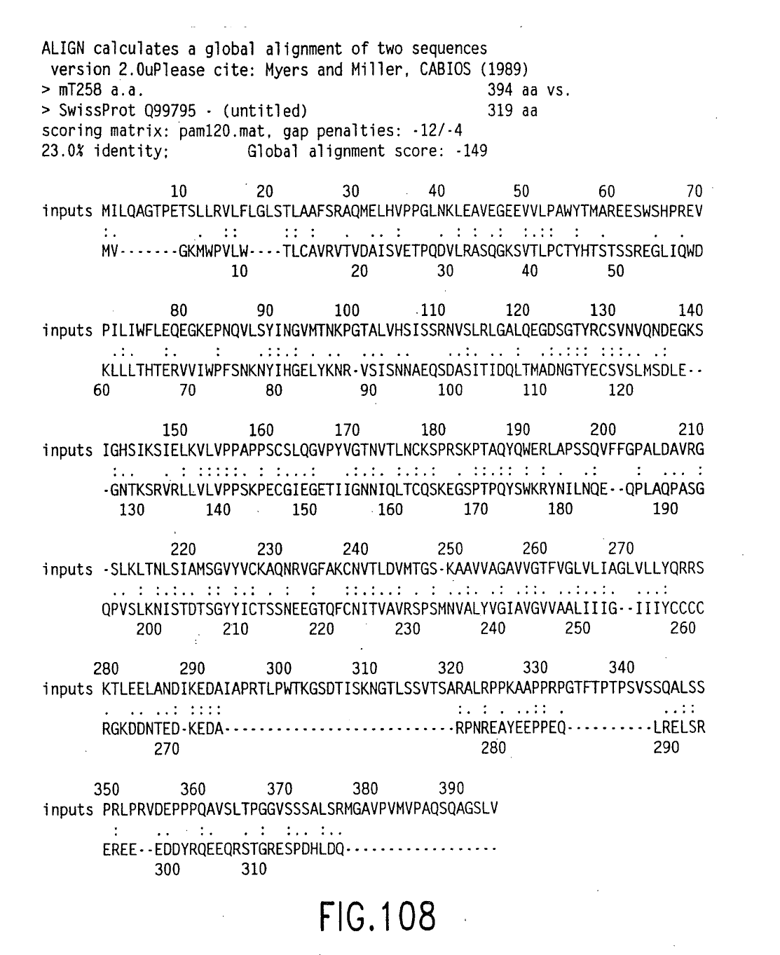

FIG. 108 shows an alignment of mouse INTERCEPT 258 amino acid sequence with the human A33 amino acid sequence. The alignment shows that there is a 23% overall amino acid sequence identity between the two sequences.

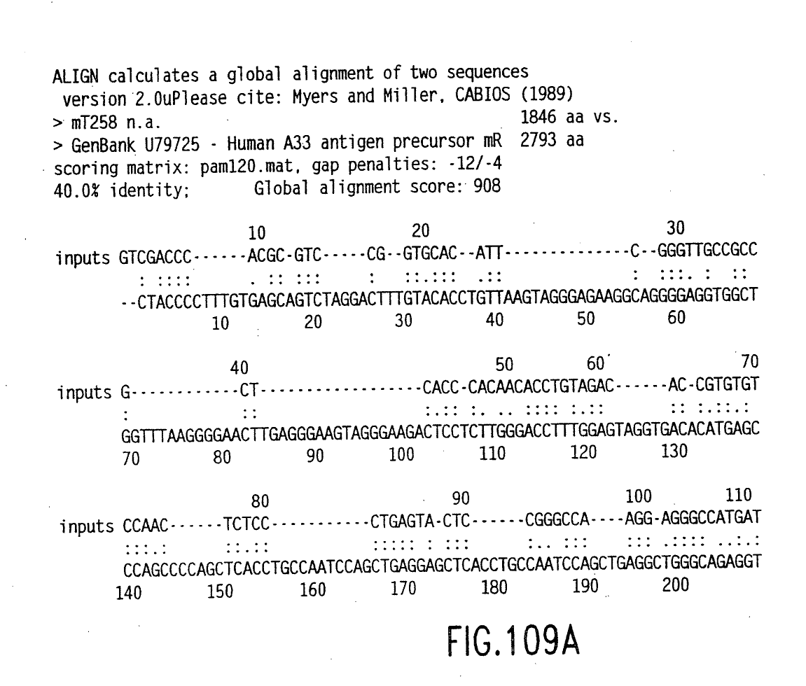

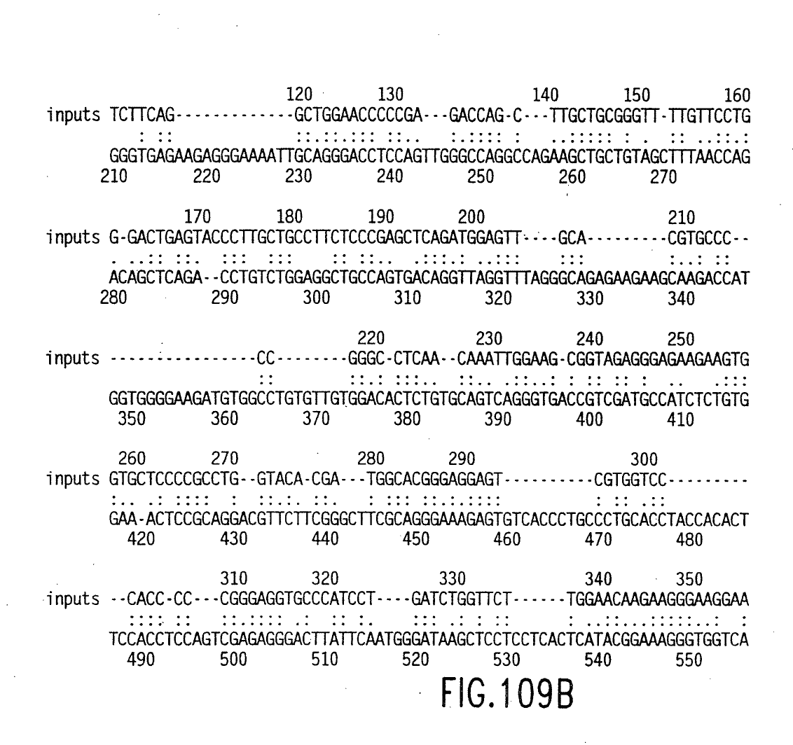

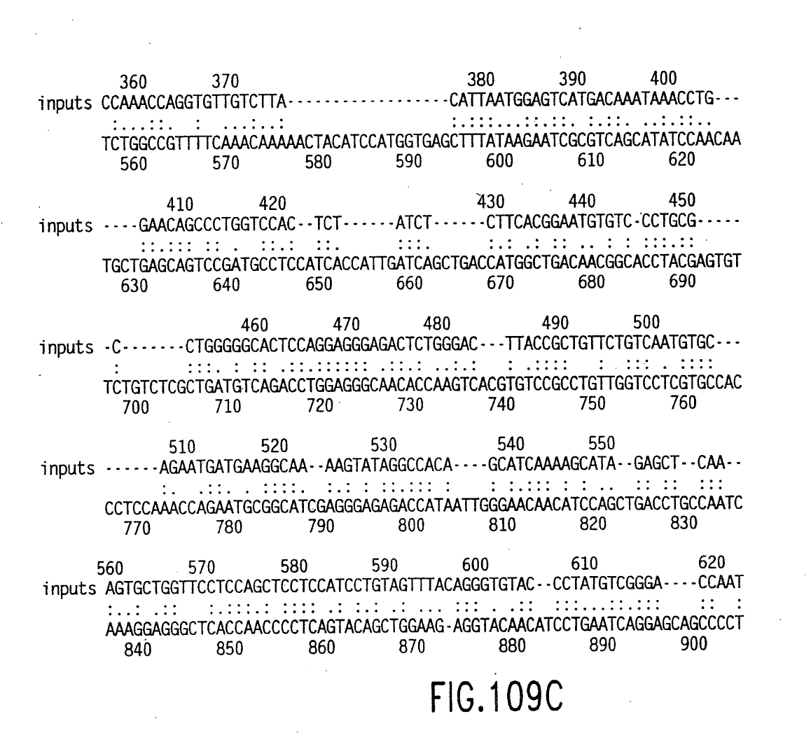

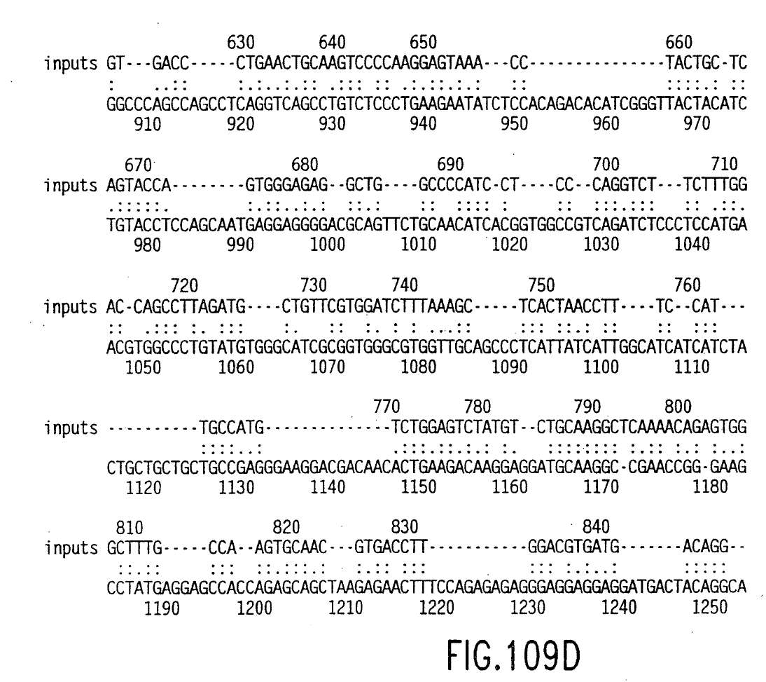

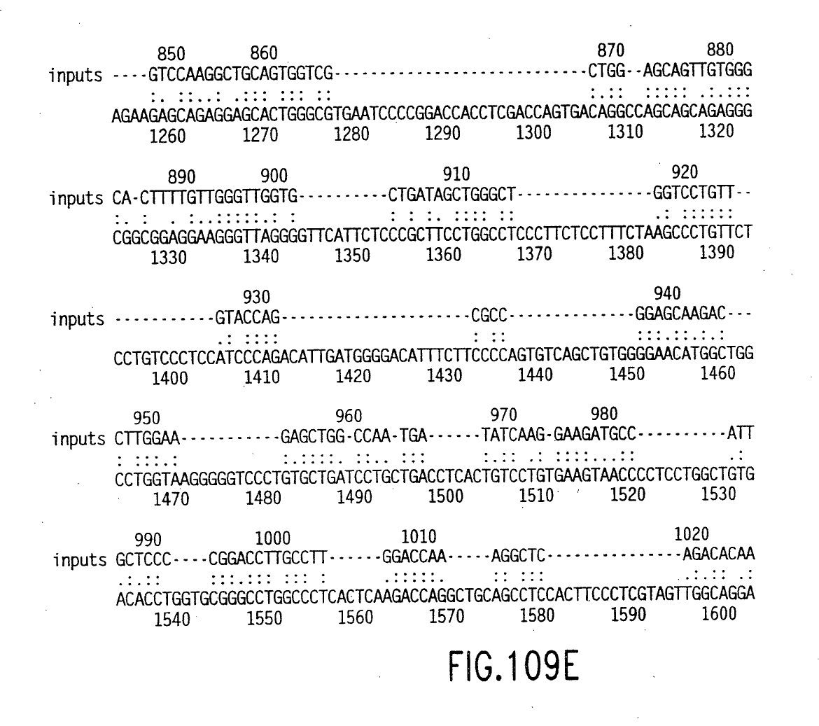

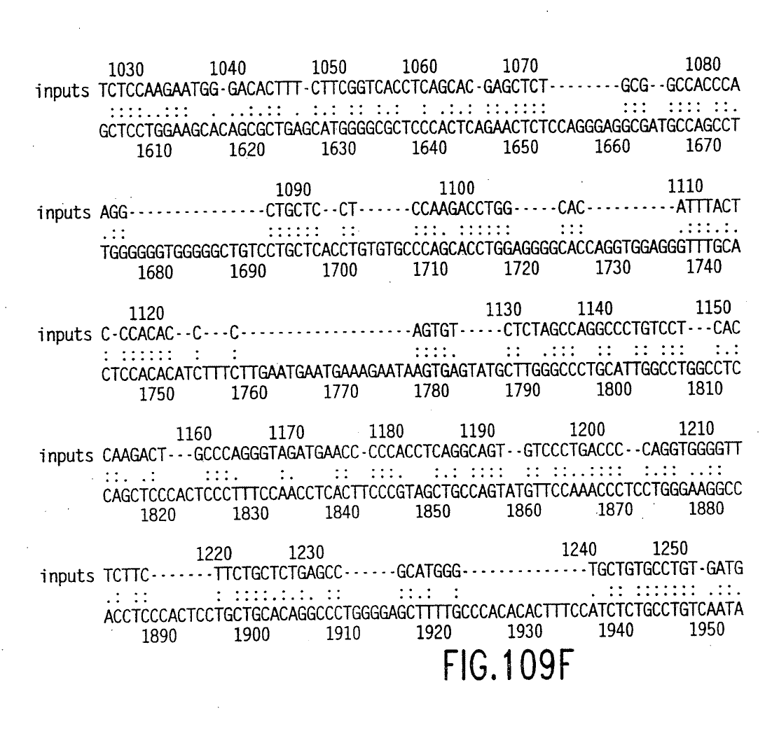

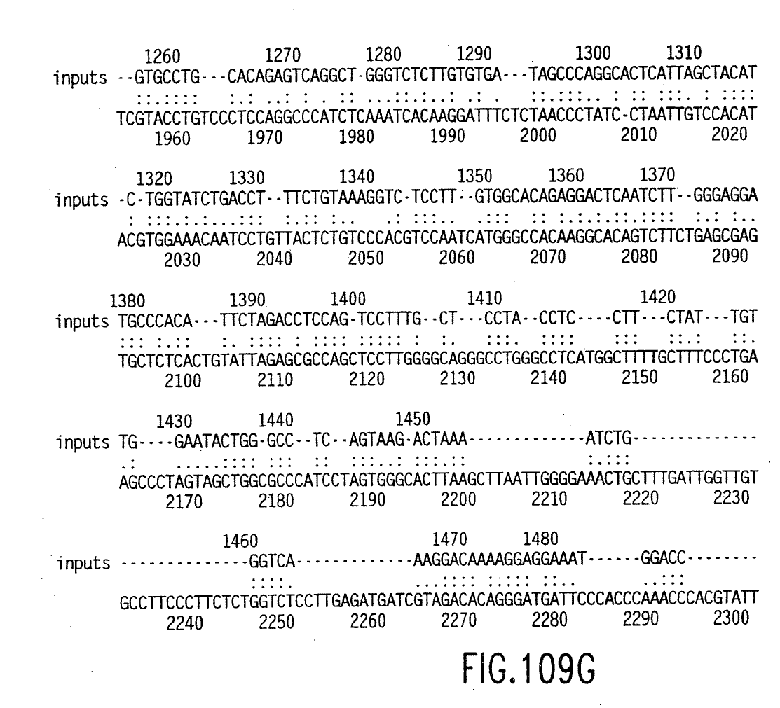

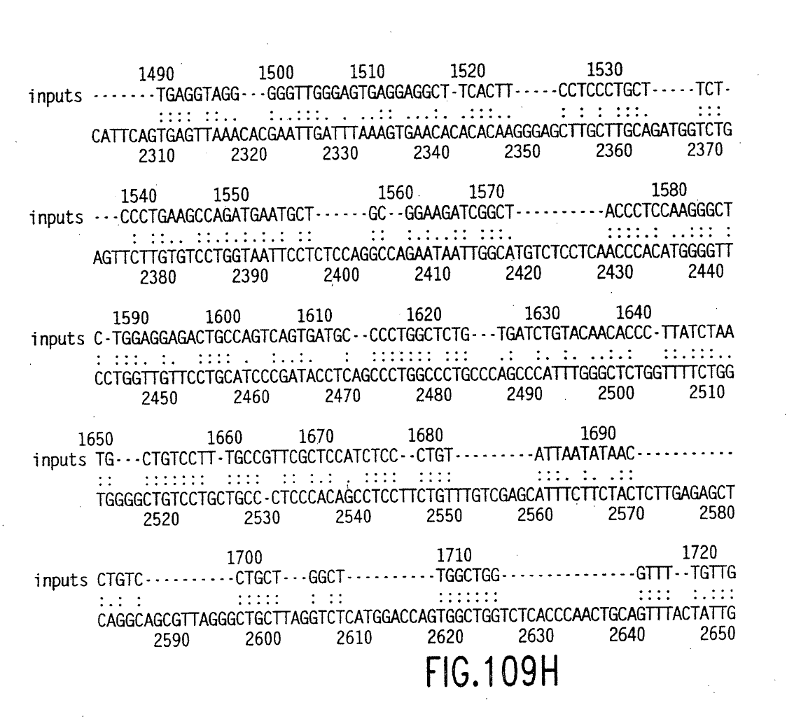

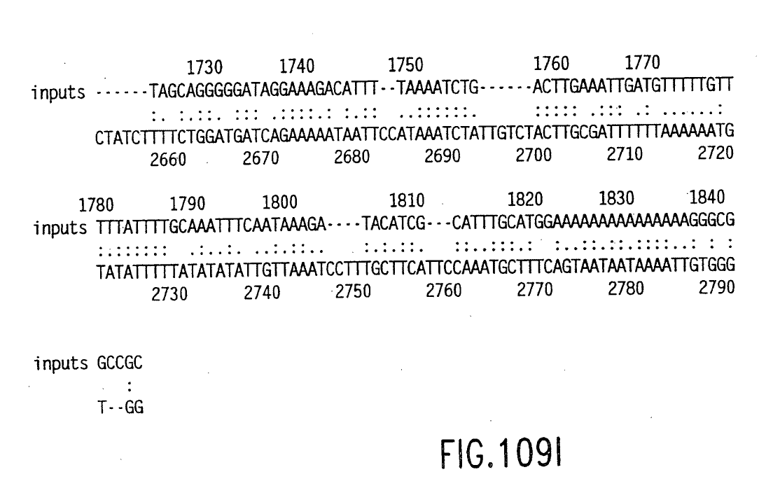

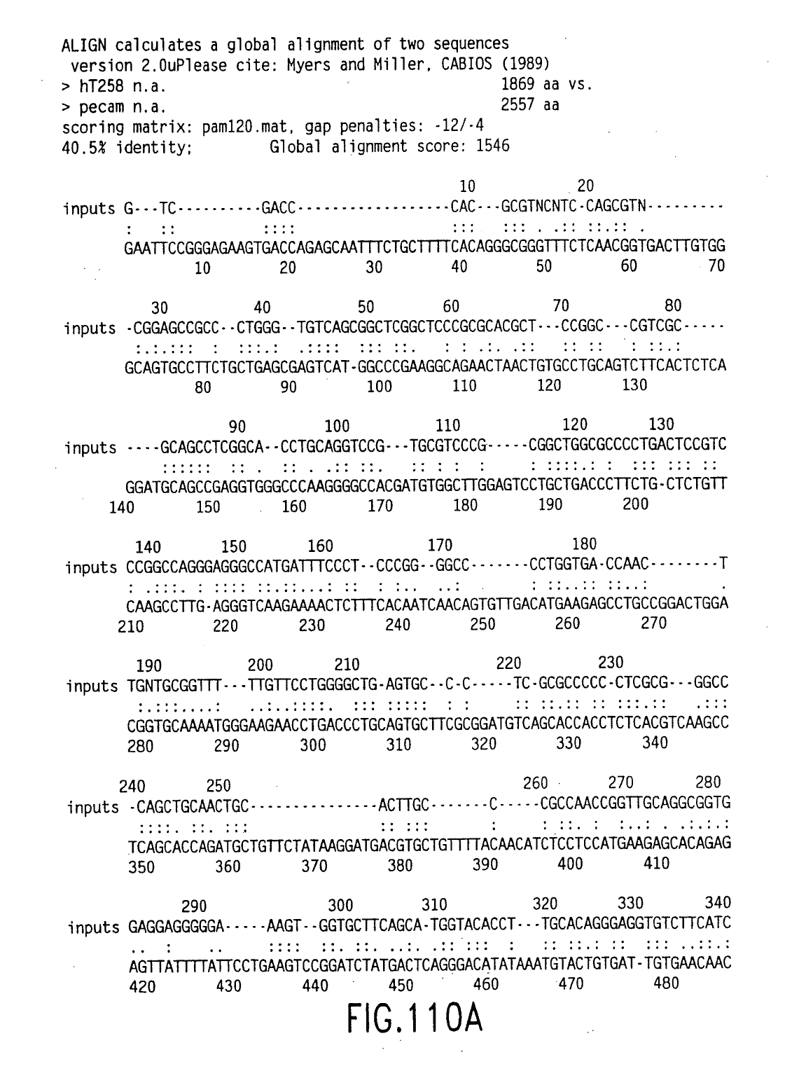

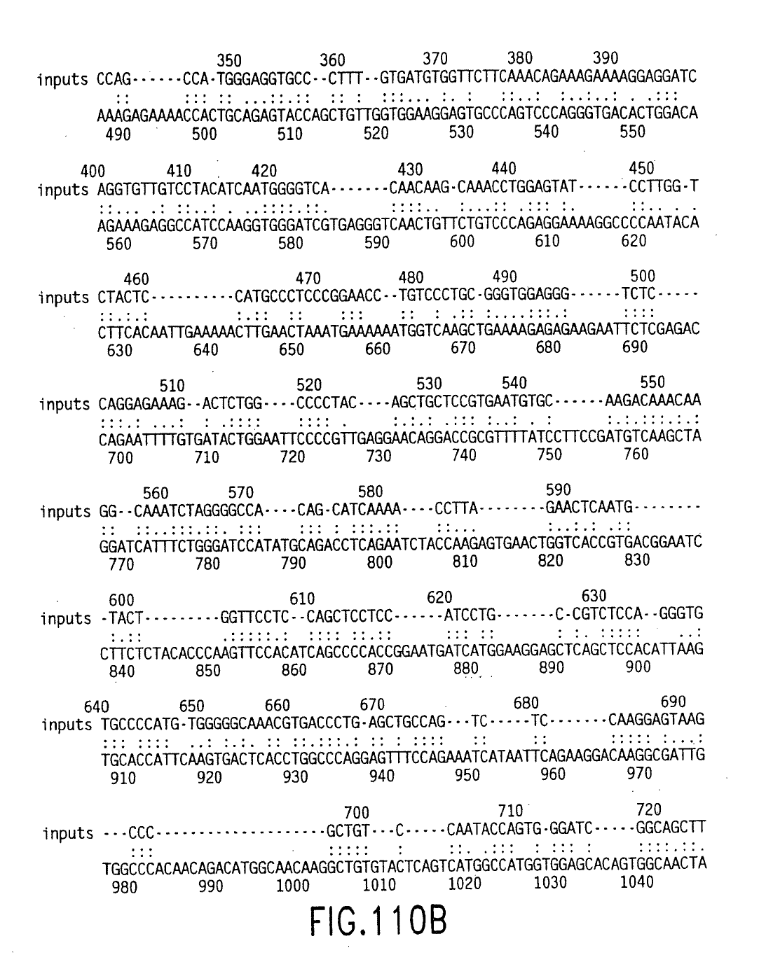

FIG. 109A-109I show an alignment of the mouse INTERCEPT 258 nucleotide sequence with that of the human A33 nucleotide sequence. The alignment shows that there is a 40% identity between these two nucleotide sequences.

Clone EpTm258, which encodes mouse INTERCEPT 258, was deposited with the American Type Culture Collection (10801 University Boulevard, Manassas, Va. 20110-2209) on Apr. 21, 1999 and assigned Accession Number 207221. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of INTERCEPT 258 Nucleic Acids, Polypeptides, and Modulators Thereof

INTERCEPT 258 was identified as being expressed in a mixed lymphocyte library. In light of this, INTERCEPT 258 nucleic acids, proteins and modulators thereof can be utilized to modulate processes involved in lymphocyte development, differentiation and activity, including, but not limited to development, differentiation and activation of T cells, including T helper, T cytotoxic and non-specific T killer cell types and subtypes, and B cells, immune functions associated with such cells, and amelioration of one or more symptoms associated with abnormal function of such cell types. Such disorders can include, but are not limited to, autoimmune disorders, such as organ specific autoimmune disorders, e.g., autoimmune thyroiditis, Type I diabetes mellitus, insulin-resistant diabetes, autoimmune anemia, multiple sclerosis, and/or systemic autoimmune disorders, e.g., rheumatoid arthritis, lupus or sclerodoma, allergy, including allergic rhinitis and food allergies, asthma, psoriasis, graft rejection, transplantation rejection, graft versus host disease, pathogenic susceptibilities, e.g., susceptibility to certain bacterial or viral pathogens, wound healing and inflammatory reactions.

INTERCEPT 258 includes one or more Ig domains. INTERCEPT 258 nucleic acids, proteins, and modulators thereof can, therefore, be used to modulate immune function, e.g., by the modulation of immunoglobulins and the formation of antibodies. For the same reason, INTERCEPT 258 nucleic acids proteins, and modulators thereof can be used to modulate immune response, leukocyte trafficking, cancer, Type I immunologic disorders, e.g., anaphylaxis and/or rhinitis, by modulating the interaction between antigens and cell receptors, e.g., high affinity IgE receptors.

INTERCEPT 258 exhibits homology to PECAM-1, a cell adhesion integrin molecule that has been shown to mediate cell-cell interactions, play an important role in bidirectional signal transduction, and may be involved in thrombotic, inflammatory and immunological disorders. As such, INTERCEPT 258 nucleic acids, proteins, and modulators thereof can be utilized to modulate cell/cell interactions and, for example, signal transduction events associated with such interactions. For example, such INTERCEPT 258 compositions and modulators thereof can be used to modulate binding of cellular factors or ECM-associated factors such as integrin and can function to modulate ligand binding to cell surface receptors. Further, such INTERCEPT 258 compositions and modulators thereof can be utilized to ameliorate at least one symptom associated with thrombotic disorders, e.g., stroke, inflammatory processes or disorders, and immune disorders.

In light of INTERCEPT 258 expression, INTERCEPT 258 nucleic acids, proteins and modulators thereof can be utilized modulate development, differentiation, proliferation and/or activity of pulmonary system cells, e.g., lung cell types, and to modulate a symptom associated with disorders of pulmonary development, differentiation and/or activity, such as lung diseases or disorders associated with abnormal pulmonary development or function, e.g., cystic fibrosis. INTERCEPT 258 nucleic acids, proteins and modulators thereof can also be utilized modulate development, differentiation, proliferation and/or activity of thyroid cells, megakaryocytes or mammary gland cells, and can further be utilized to ameliorate at least one symptom of disorders associated with, abnormal thyroid function, e.g., thyroiditis or Grave's disease, abnormal megakaryocyte differentiation or function, e.g., anemias or leukemias, hematological diseases such as thrombocytopenia, platelet disorders and bleeding disorders, such as hemophilia or abnormal mammary development or function.

INTERCEPT 258 nucleic acids, polypeptides, or modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedulla sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy), acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

INTERCEPT 258 nucleic acids, polypeptides, or modulators thereof can also be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

INTERCEPT 258 nucleic acids, proteins, and modulators thereof can still further be utilized to modulate development, differentiation proliferation and/or activity of cells involved in kidney or heart formation and function. In addition, such compositions and modulators thereof can be utilized to ameliorate at least one symptom of disorders associated with abnormal kidney or heart formation or function, including, but not limited to nephritis, coronary disease, atherosclerosis and plaque formation.

INTERCEPT 258 expression indicates that INTERCEPT 258 is involved, in addition to the above, in such processes as thermogenesis, adipocyte function, and vascularization. As such, INTERCEPT 258 nucleic acids, proteins, and modulators thereof can be utilized to modulate such processes as well as for ameliorating at least one symptom associated with such processes. Such disorders include, but are not limited to obesity, regulation of body temperature, and disorders involving abnormal vascularization, e.g., vascularization of solid tumors.

In further light of INTERCEPT 258 expression, as well as in light of its homology to A33 antigen, INTERCEPT 258 nucleic acids, proteins and modulators thereof can be utilized to modulate cell proliferation, including, for example, epithelial, e.g., gastrointestinal tract epithelial cell proliferation, and to ameliorate at least one symptom of cell proliferative disorders such as cancer, and, in particular, chronic myelogenous leukemia, colon cancers, small bowel epithelium cancers and other gastrointestinal tract cancers. Further, INTERCEPT 258 expression can be utilized as a marker for specific tissues (e.g., vascularized tissues) and/or cells (e.g., endothelial cells) in which INTERCEPT 258 is expressed. INTERCEPT 258 nucleic acids can also be utilized for chromosomal mapping.

### Human TANGO 204

A cDNA encoding TANGO 204 was identified by analyzing the sequences of clones present in a human lung cDNA library.

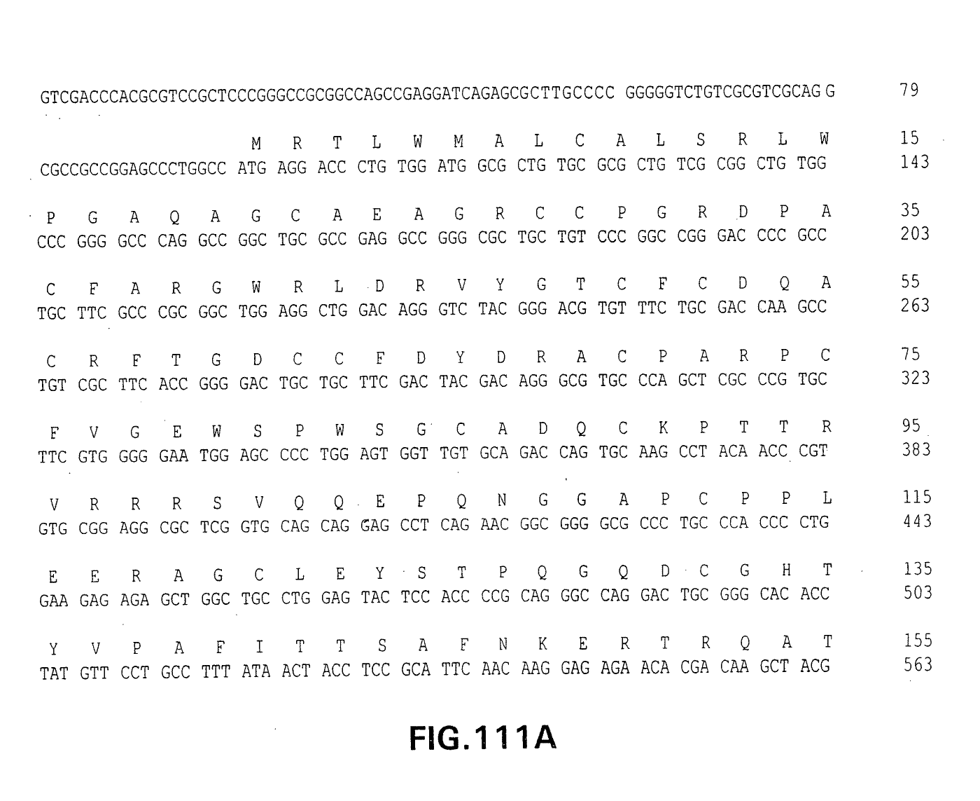

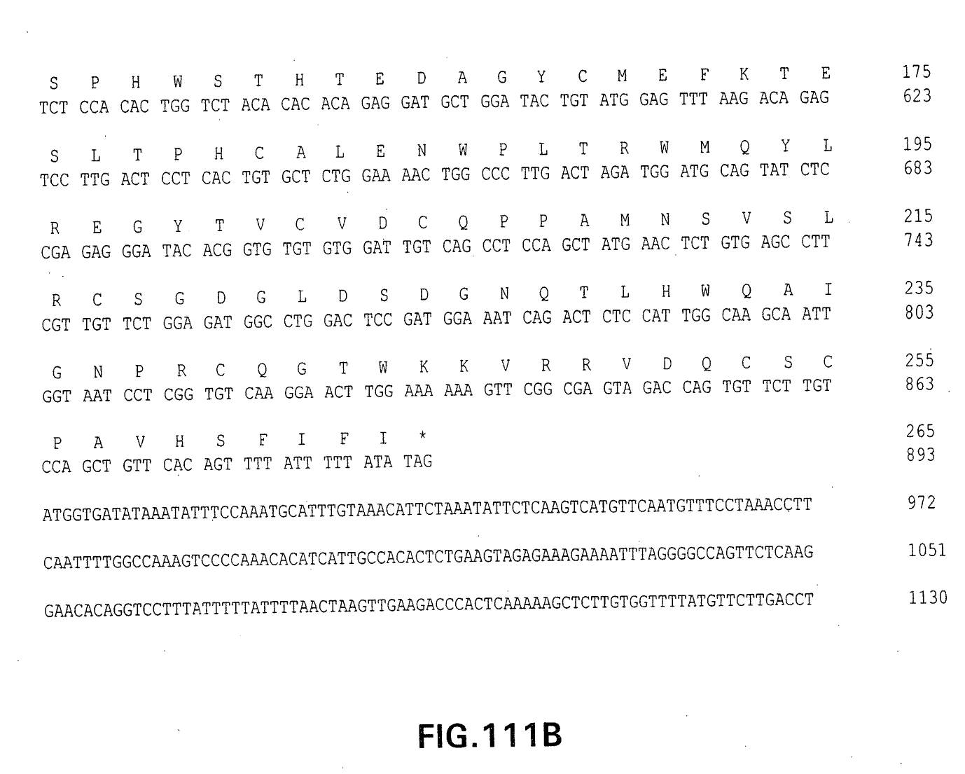

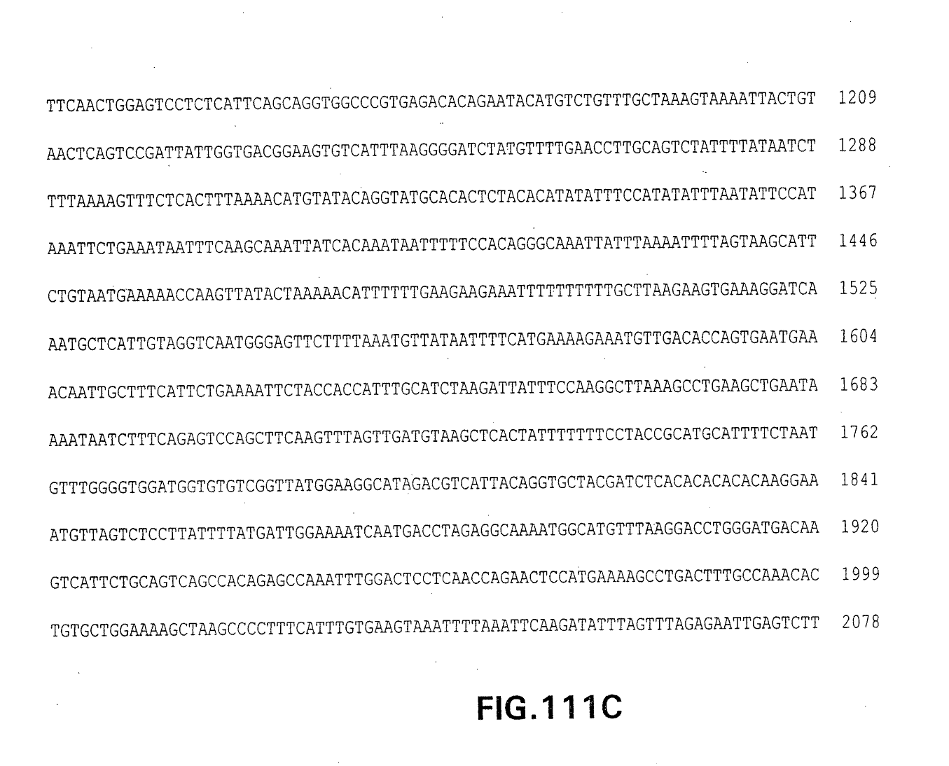

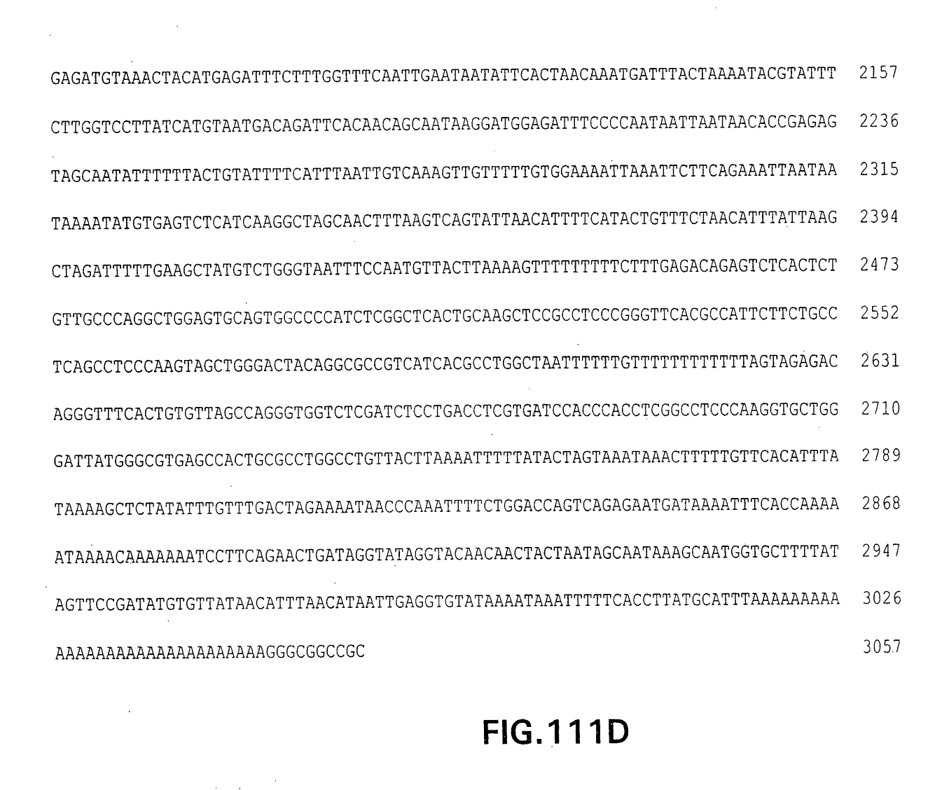

This analysis led to the identification of a clone, Athu204c, encoding full-length human TANGO 204. The cDNA of this clone is 3057 nucleotides long (FIG. 111A-111D; SEQ ID NO:79). The 792 nucleotide open reading frame of this cDNA (nucleotides 99-890 of SEQ ID NO:79) encodes a 264 amino acid protein (SEQ ID NO:80).

In one embodiment of a nucleotide sequence of human TANGO 204 the nucleotide at position 170 is a guanine (G). In this embodiment, the amino acid at position 24 is glutamate (E). In another embodiment of a nucleotide sequence of human TANGO 204 the nucleotide at position 170 is a cytosine (C). In this embodiment, the amino acid at position 24 is aspartate (D) In another embodiment of a nucleotide sequence of human TANGO 204, the nucleotide at position 335 is an adenine (A). In this embodiment, the amino acid at position 79 is a glutamate (E). In another embodiment of a nucleotide sequence of human TANGO 204, the nucleotide at position 335 is a cytosine (C). In this embodiment, the amino acid at position 79 is aspartate (D). In another embodiment of a nucleotide sequence of human TANGO 204, the nucleotide at position 410 is a guanine (G). In this embodiment, the amino acid at position 104 is a glutamate (E). In another embodiment of a nucleotide sequence of human TANGO 204, the nucleotide at position 410 is a cytosine (C). In this embodiment, the amino acid at position 104 is aspartate (D).

The presence of a methionine residue at amino acid residue positions 6, 170, 192, and 210 of SEQ ID NO:80 indicates that there can be alternative forms of human TANGO 204 of 259 amino acids, 95 amino acids, 73 amino acids, and 55 amino acids, respectively.

Another embodiment of the invention includes isolated nucleic acid molecules comprising a polynucleotide having a nucleotide sequence encoding the polypeptide having the human TANGO 204 polypeptide sequence, but lacking the N-terminal methionine residue. In this embodiment, the nucleotide sequence of human TANGO 204, nucleotides 102-890, encodes the human TANGO 204 amino acid sequence from amino acids 2-264 of SEQ ID NO:80.

The signal peptide prediction program SIGNALP (Nielsen et al. (1997) Protein Engineering 10:1-6) predicted that human TANGO 204 includes a 20 amino acid signal peptide (amino acid 1 to about amino acid 20 of SEQ D NO:80) preceding the mature human TANGO 204 protein (corresponding to about amino acid 21 to amino acid 264 of SEQ ID NO:80).

In one embodiment, a TANGO 204 protein contains a signal sequence of about amino acids 1-20. In certain embodiments, a TANGO 204 family member has the amino acid sequence, and the signal sequence is located at amino acids 1 to 18, 1 to 19, 1 to 20, 1 to 21 or 1 to 22. In such embodiments of the invention, the domains and the mature protein resulting from cleavage of such signal peptides are also included herein. For example, the cleavage of a signal sequence consisting of amino acids 1 to 20 results in a mature TANGO 204 protein corresponding to amino acids 21 to 264 of SEQ ID NO:80. The signal sequence is normally cleaved during processing of the mature protein.

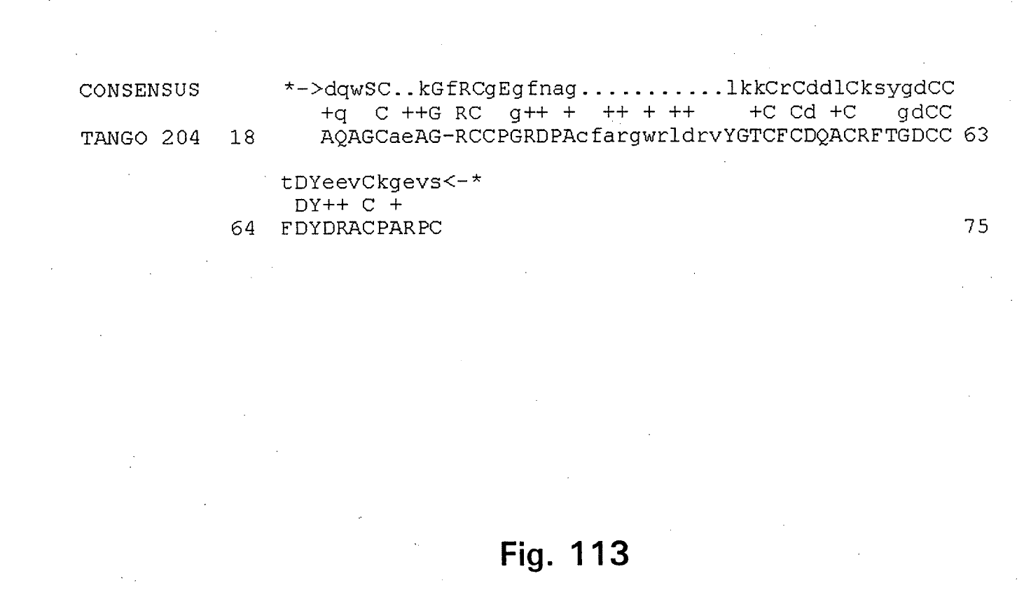

TANGO 204 family members can also include a somatomedin B domain. Somatomedin B domains are present in plasma cell glycoprotein PC-1 and placental protein 11. Somatomedin B domains have the sequence Cys-Xaa₆-C-Xaa₉-Cys-Xaa-Cys-Xaa₃-Cys-Xaa₅-Cys-Cys-Xaa₅-Cys (where Xaa can be any amino acid). The most highly conserved portion of the somatomedin B domain has the sequence Cys-Xaa-Cys-Xaa₃-C-Xaa₄-Cys-Cys-Xaa₄-Cys (where Xaa can be any amino acid). The cysteine residues within the domain are all likely involved in disulfide bonds. A consensus somatomedin B domain has the sequence. This consensus sequence is shown in FIG. 113 where the more conserved residues in the consensus sequence are indicated by uppercase letters and the less conserved residues in the consensus sequence are indicated by lowercase letters. The somatomedin B domain of human TANGO 204 is located at amino acids 18-75.

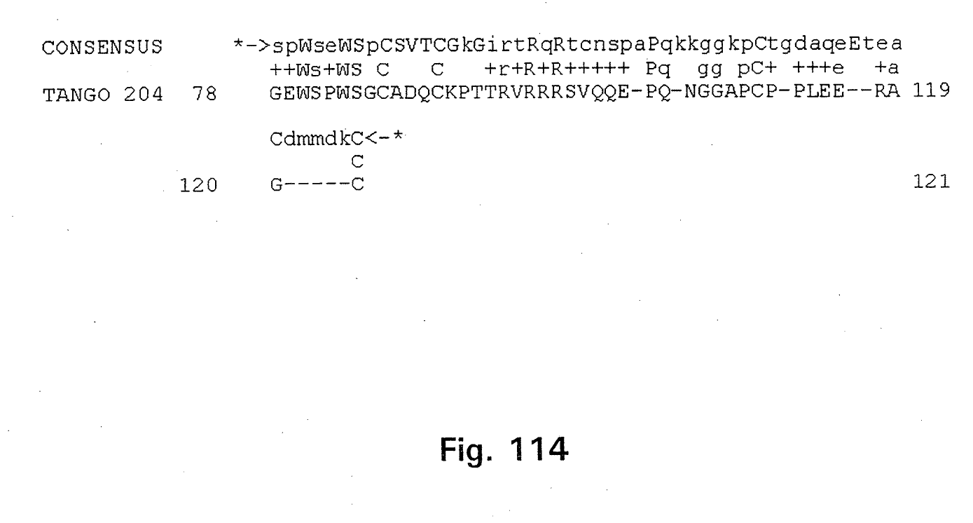

TANGO 204 family members can also include a thrombospondin type I domain. A consensus thrombospondin type 1 domain has the sequence depicted in the alignment shown in FIG. 114. This consensus sequence is shown in FIG. 114 where the more conserved residues in the consensus sequence are indicated by uppercase letters and the less conserved residues in the consensus sequence are indicated by lowercase letters. The thrombospondin type 1 domain of human TANGO 204 is located at amino acids 78-121. Thrombospondin type 1 domains can include the sequence CS(A/V)TCG and the sequence W(S/G)XW.

Human TANGO 204 that has not been post-translationally modified is predicted to have a molecular weight of 29.6 kDa prior to cleavage of its signal peptide and a molecular weight of 27.3 kDa subsequent to cleavage of its signal peptide.

Human TANGO 204 includes a somatomedin B domain at amino acids 18-75 of SEQ ID NO:80. FIG. 113 depicts an alignment of the somatomedin B domain of human TANGO 204 with a consensus somatomedin B domain derived from a hidden Markov model. Human TANGO 204 also includes a thrombospondin type I domain at amino acids 78-221 of SEQ ID NO:80. FIG. 114 depicts an alignment of the thrombospondin type I domain of human TANGO 204 with a consensus thrombospondin type I domain derived from a hidden Markov model.

An N-glycosylation site is present at amino acids 227-230. A cAMP and cGMP-dependent protein kinase phosphorylation site is present at amino acids 97-100. Protein kinase C phosphorylation sites are present at amino acids 93-95, 214-216, and 243-245. A casein kinase II phosphorylation site is present at amino acids 161-164. N-myristoylation sites are present at amino acids 17-22, 48-53, 129-134, and 236-241. A growth factor and cytokine receptor family signature sequence is present at amino acids 78-84. A somatomedin B domain signature sequence is present at amino acids 50-70. Clone Athu204c, which encodes human TANGO 204, was deposited as fthv204c with the American Type Culture Collection (10801 University Boulevard, Manassas, Va. 20110-2209) on Apr. 2, 1999 and assigned Accession Number 207192. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

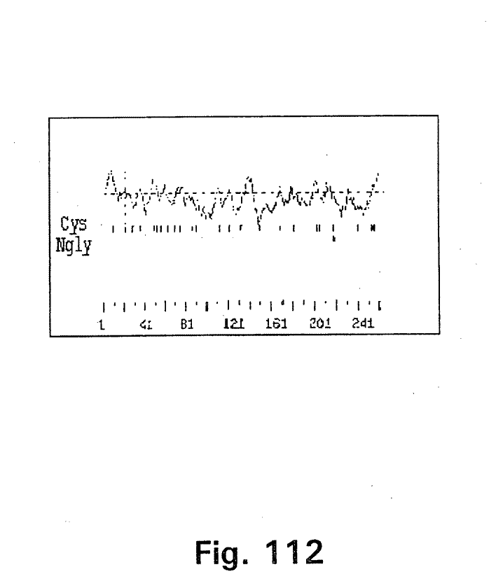

FIG. 112 depicts a hydropathy plot of human TANGO 204. The hydropathy plot indicates that human TANGO 204 has a signal sequence at its amino terminus and a hydrophobic region at its carboxy terminus, suggesting that TANGO 204 is a membrane-associated protein.

TANGO 204 is likely membrane-associated through its hydrophobic carboxy-terminus. The last nine amino acids of human TANGO 204 (amino acids 256-264) are very hydrophobic. Further, there are two pairs of basic residues near the hydrophobic C-terminus (KK at amino acids 245-246 and RR at amino acids 248-249). These residues can serve as proteolytic cleavage sites. Thus, cleavage at either pair of basic residues can release a soluble form of TANGO 204 (amino acid 20-244, 20-245, 20-246, 20-287, 20-288, or 20-249). In addition, there is a RRR sequence at amino acids amino acids 97-99, and proteolytic cleavage at this sequence can release a soluble form of TANGO 204 (amino acids 20-96, 20-97, 20-98, or 20-99). The presence of a somatomedin B domain sequence within human TANGO 204 is consistent with TANGO 204 being a membrane-associated protein.

The human TANGO 204 gene maps to chromosome 8q between D8S257 and D8S508 based on the homology between a portion of human TANGO 204 and Genbank Accession Number G25656, which is reported to map to this position.

### Mouse TANGO 204

A mouse homolog of human TANGO 204 was identified. A cDNA encoding mouse TANGO 204 was identified by analyzing the sequences of clones present in a stimulated mouse osteoblast cDNA library.

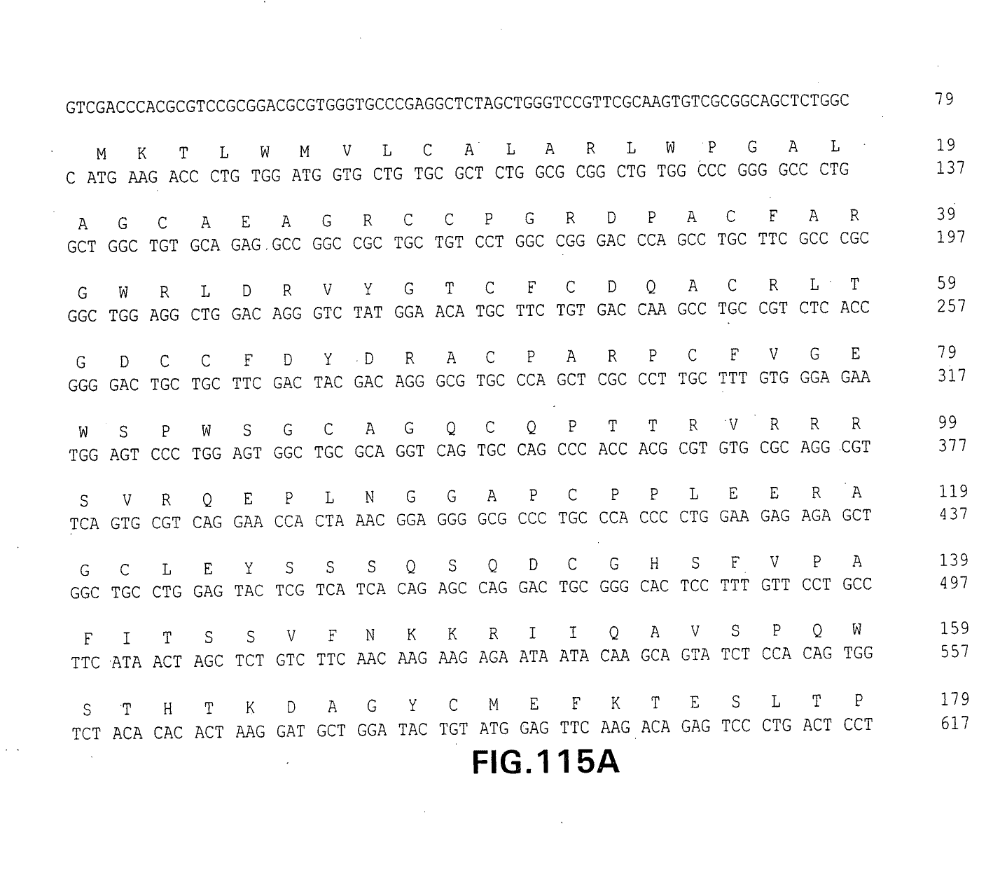

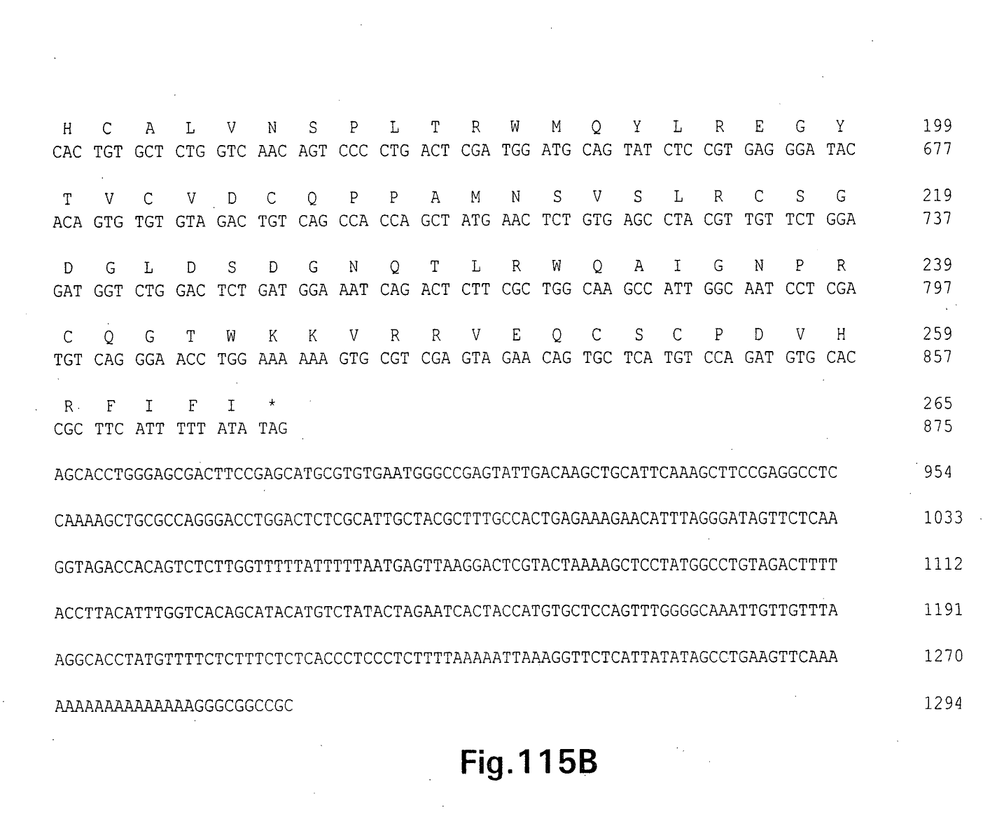

This analysis led to the identification of a clone, Atmoa043g03, encoding full-length mouse TANGO 204. The cDNA of this clone is 1294 nucleotides long (FIG. 115A-115B; SEQ ID NO:81). The 792 nucleotide open reading frame of this cDNA (nucleotides 81-872 of SEQ ID NO:81) encodes a 264 amino acid protein (SEQ ID NO:82).

In one embodiment of a nucleotide sequence of mouse TANGO 204 the nucleotide at position 152 is a guanine (G). In this embodiment, the amino acid at position 24 is glutamate (E). In another embodiment of a nucleotide sequence of mouse TANGO 204, the nucleotide at position 152 is a cytosine (C). In this embodiment, the amino acid at position 24 is aspartate (D). In another embodiment of a nucleotide sequence of mouse TANGO 204, the nucleotide at position 392 is an adenine (A). In this embodiment, the amino acid at position 104 is a glutamate (E). In another embodiment of a nucleotide sequence of mouse TANGO 204, the nucleotide at position 392 is a cytosine (C). In this embodiment, the amino acid at position 104 is aspartate (D). In another embodiment of a nucleotide sequence of mouse TANGO 204, the nucleotide at position 425 is an adenine (A). In this embodiment, the amino acid at position 116 is a glutamate (E). In another embodiment of a nucleotide sequence of mouse TANGO 204, the nucleotide at position 425 is a cytosine (C). In this embodiment, the amino acid at position 116 is aspartate (D).

The presence of a methionine residue at amino acid residue positions 6, 170, 192, and 210 indicates that there can be alternative forms of mouse TANGO 204 of 259 amino acids, 95 amino acids, 73 amino acids, and 55 amino acids, respectively.

Another embodiment of the invention includes isolated nucleic acid molecules comprising a polynucleotide having a nucleotide sequence encoding the polypeptide having the mouse TANGO 204 polypeptide sequence, but lacking the N-terminal methionine residue. In this embodiment, the nucleotide sequence of mouse TANGO 204, nucleotides 84-872, encodes the mouse TANGO 204 amino acid sequence comprising amino acids 2-264 of SEQ ID NO:82.

The signal peptide prediction program SIGNALP (Nielsen et al. (1997) Protein Engineering 10:1-6) predicted that mouse TANGO 204 includes a 20 amino acid signal peptide (amino acid 1 to about amino acid 20 of SEQ ID NO:82) preceding the mature mouse TANGO 204 protein (corresponding to about amino acid 21 to amino acid 264 of SEQ ID NO:82).

Mouse TANGO 204 that has not been post-translationally modified is predicted to have a molecular weight of 29.5 kDa prior to cleavage of its signal peptide and a molecular weight of 27.2 kDa subsequent to cleavage of its signal peptide.

Mouse TANGO 204 includes a somatomedin B domain at amino acids 18-75 of SEQ ID NO:82 and a thrombospondin type I domain at amino acids 78-121 of SEQ ID NO:82.

In situ tissue screening was performed on mouse adult and embryonic tissue to analyze the expression of mouse TANGO 204 mRNA. In summary, embryonic expression was observed in a number of tissues and organs. Most noticeable was the expression in the eye, lung, stomach, intestine, and the tissue just under the skin in the feet which outlines the digits. Expression was also associated with some developing bone and cartilage structures such as the ear, nose, and spinal column. Expression decreased to background levels in most of these tissue and was observed in only a few adult tissues; eye, kidney, and adrenal gland.

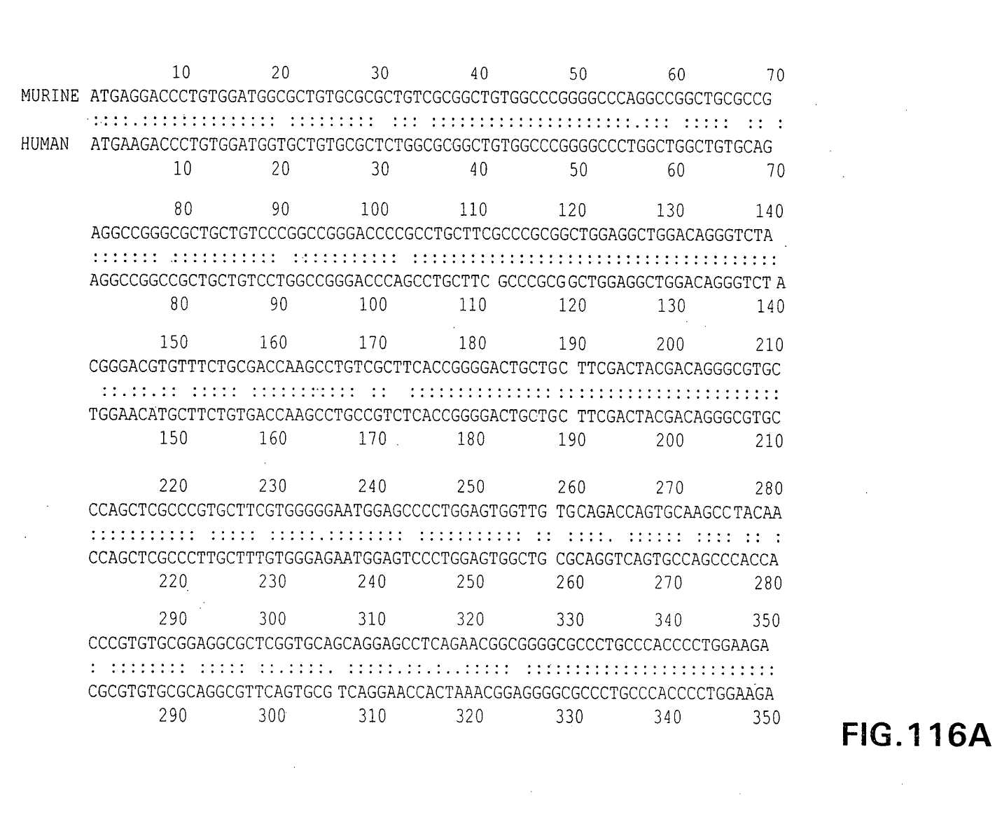

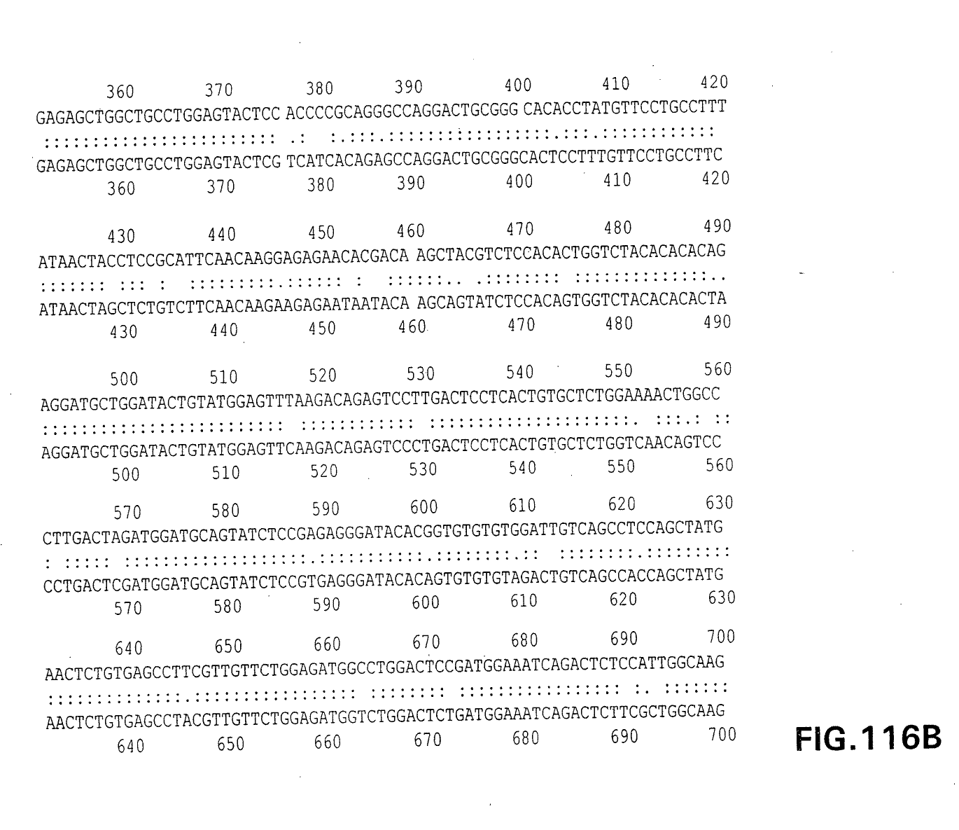

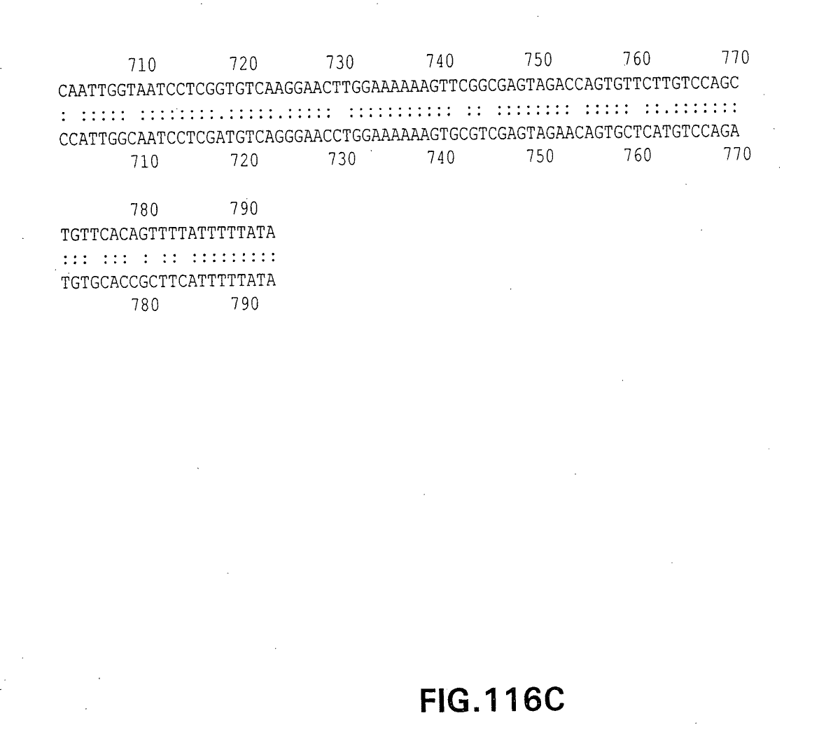

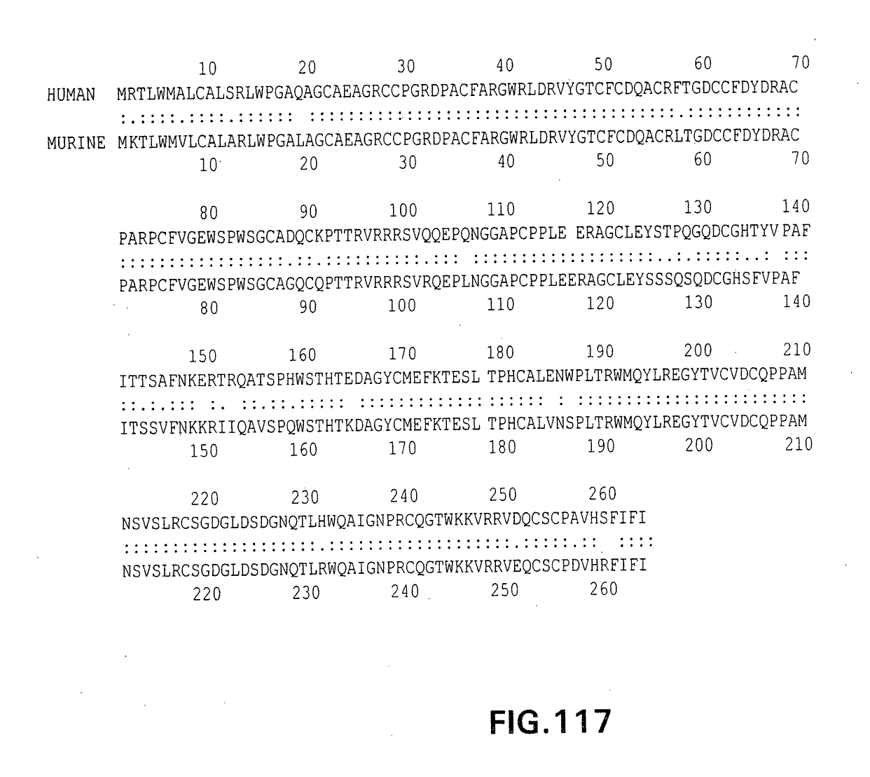

Human and mouse TANGO 204 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 89.4%. The human and mouse TANGO 204 full length cDNAs are 78.4% identical, as assessed using the same software and parameters as indicated. In the respective ORFs, calculated in the same fashion as the full length cDNAs, human and mouse TANGO 204 are 87.5% identical. The nucleotide sequence and amino acid sequence alignments of human and mouse TANGO 204 can be found in FIG. 116A-116C and FIG. 117, respectively.

The mouse TANGO 204 gene was mapped to mouse using the Genebridge 4 Radiation hybrid mapping panel with GACAAGCTGCATTCAAAGCTTCC as the forward primer and CTGGAGCACATGGTAGTGATTC as the reverse primer. The mouse TANGO. 204 gene maps to chromosome 1. Flanking markers for this region are D1Mit430 and D1Mit119. Mapping by synteny reveals that human TANGO 204 maps to human chromosome 8q. The CCAL1 (chondrocalcinosis 1) locus also maps to this region of the human chromosome. The OPRK (opiate receptor) gene also maps to this region of the human chromosome. The tb (tumbler), fz (fuzzy) loci also map to this region of the mouse chromosome. The tb (tumbler), fz(fuzzy) genes also map to this region of the mouse chromosome.

Clone Atmoa043g03, which encodes mouse TANGO 204, was deposited as Atmoa43g3 with the American Type Culture Collection (10801 University Boulevard, Manassas, Va. 20110-2209) on Apr. 2, 1999 and assigned Accession Number 207189. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Use of TANGO 204 Nucleic Acids, Polypeptides, and Modulators Thereof

TANGO 204 polypeptides, nucleic acids, and modulators thereof, can be used to modulate the function, morphology, proliferation and/or differentiation of cells in the tissues in which it is expressed. TANGO 204 includes a thrombospondin type 1 domain. Known proteins having this domain play a role in blood coagulation, cellular proliferation, cellular adhesion, migration of tumor cells, migration of normal cells, and angiogenesis. The thrombospondin type 1 domain can mediate interaction with matrix macromolecules, including heparan sulfate, proteoglycans, fibronectin, laminin, and collagen. TANGO 204 polypeptides, nucleic acids, and modulators thereof can be used to treat disorders of blood clotting, angiogenesis (e.g., to reduce tumor growth by inhibiting angiogenesis or promote wound healing by stimulating angiogenesis), and cancer. TANGO 204 polypeptides, nucleic acids, and modulators thereof can also be used to treat connective tissue disorders (Marfan syndrome and osteogenesis imperfecta). TANGO 204 includes a somatomedin B domain. Known proteins having this domain are involved in regulation of plasminogen activator inhibitor, a protein which regulates activity of plasmin, a protein involved in ovulation, angiogenesis, neoplasia, wound healing, embryonic development, and inflammation. Thus, TANGO 204 polypeptides, nucleic acids, and modulators thereof can also be used to treat disorders of ovulation. In addition, such molecules can be used to treat disorders associated with proteases in cardiovascular tissue, disorders of complement activation, and disorders of fibrinolysis.

With respect to angiogenisis in particular, angiogenesis is also involved in pathological conditions including the growth and metastasis of tumors. In fact, tumor growth and metastasis have been shown to be dependent on the formation of new blood vessels. Accordingly, TANGO 204 polypeptides, nucleic acids and/or modulators thereof can be used to modulate angiogenesis in proliferative disorders such as cancer, (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, and retinoblastoma.

TANGO 204 polypeptides, nucleic acids, and modulators thereof, can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which TANGO 204 is expressed include, for example, eye, stomach, intestine, cortex adrenal gland, kidney, developing bone and cartilage structures such as the ear, nose, and spinal column, and the pericardium surrounding the heart.

In another example, because TANGO 204 is expressed in the pericardium surrounding the heart TANGO 201 polypeptides, nucleic acids, or modulators thereof, can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

Because TANGO 204 is expressed in the kidney, the TANGO 204 polypeptides, nucleic acids and/or modulators thereof can be used to modulate the function, morphology, proliferation and/or differentiation of cells in the tissues in which it is expressed. Such molecules can also be used to treat disorders associated with abnormal or aberrant metabolism or function of cells in the tissues in which it is expressed. Such molecules can be used to treat or modulate renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As TANGO 204 exhibits expression in the small intestine, TANGO 204 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

As mouse TANGO 204 was originally identified in an osteoblast cDNA library, TANGO 204 nucleic acids, proteins, and modulators thereof can be used to modulate the proliferation, activation, development, differentiation, and/or function of osteoblasts. Thus, TANGO 204 nucleic acids, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

As human TANGO 204 was originally identified in a lung cDNA library, human TANGO 204 nucleic acids, proteins, and modulators thereof can be used to modulate the proliferation, activation, development, differentiation, and/or function of lung cells. Thus, TANGO 204 polypeptides, nucleic acids, or modulators thereof, can be used to treat pulmonary (lung) disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another example, TANGO 204 polypeptides, nucleic acids, or modulators thereof, can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

### Human TANGO 206

A cDNA encoding human TANGO 206 was identified by analyzing the sequences of clones present in a human osteoblast cDNA library.

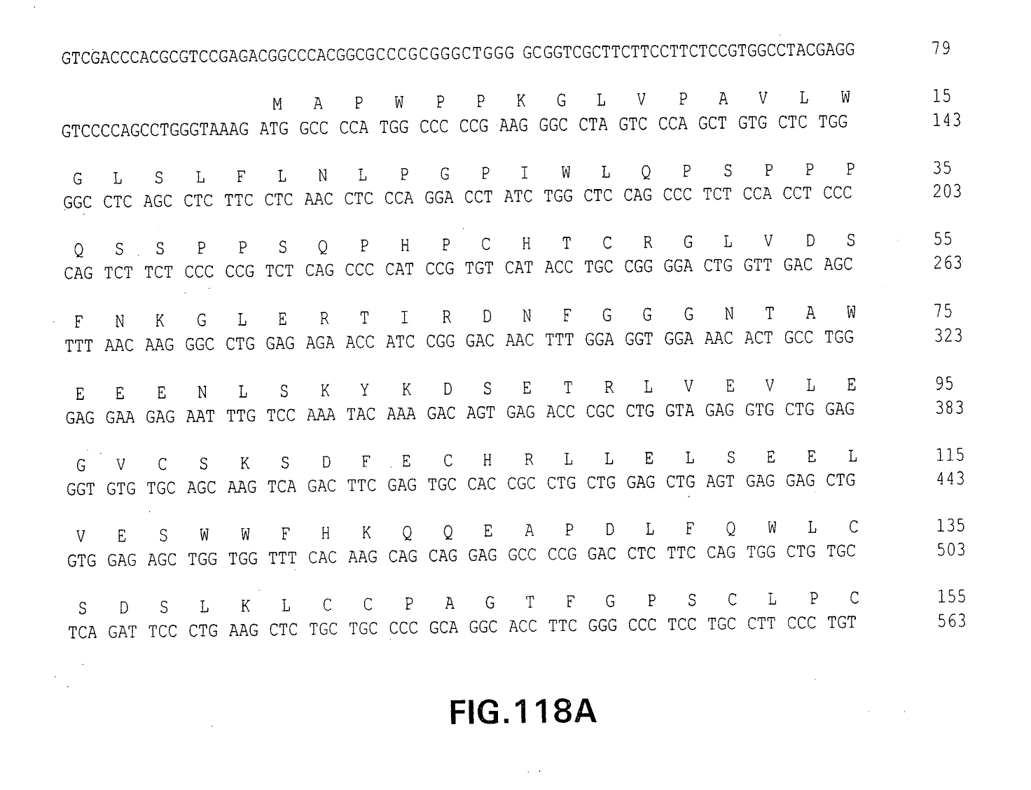

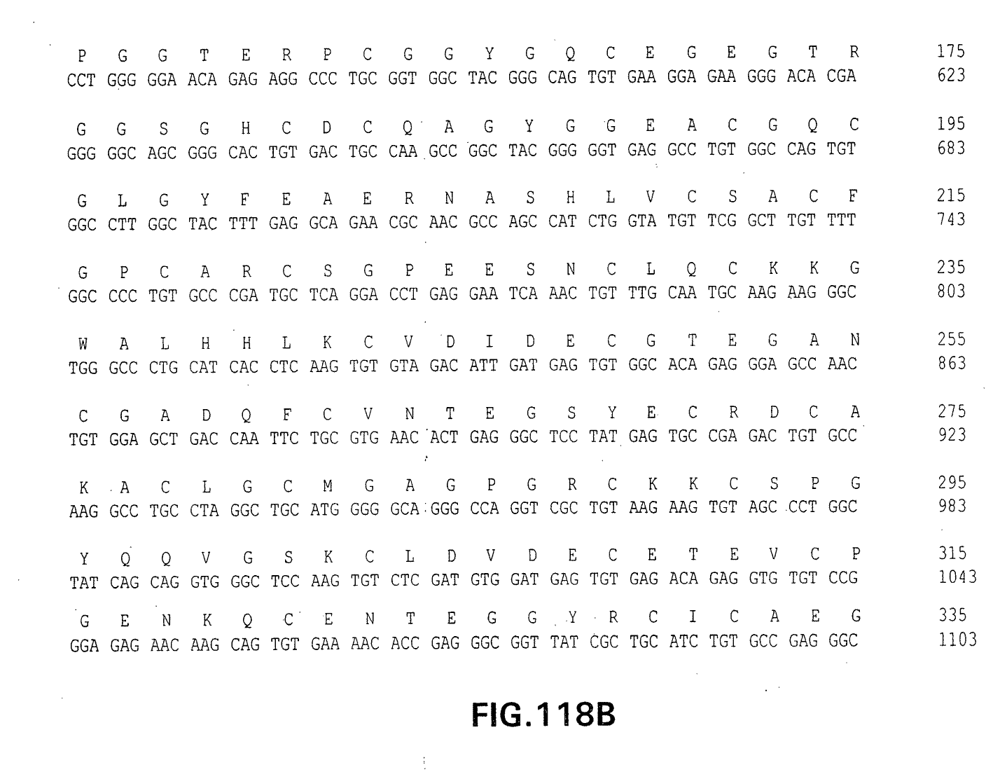

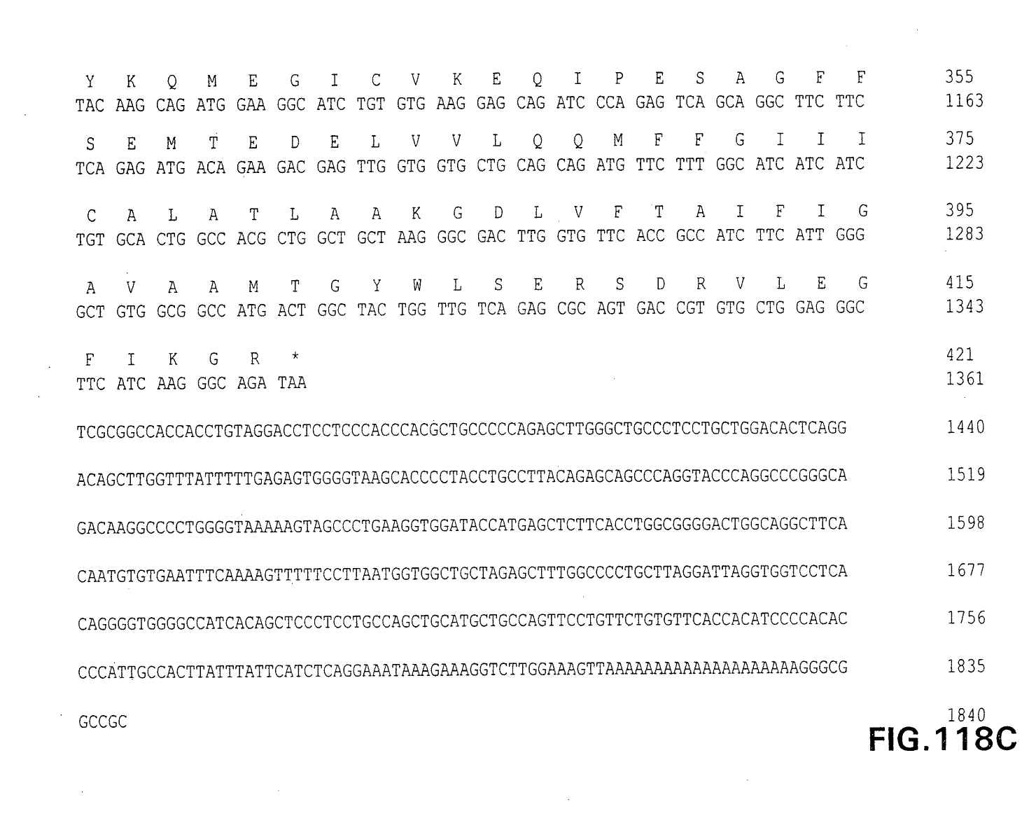

This analysis led to the identification of a clone, Athoc49b12, encoding full-length human TANGO 206. The cDNA of this clone is 1840 nucleotides long (FIG. 118A-118C; SEQ ID NO:83). The 1260 nucleotide open reading frame of this cDNA (nucleotides 99-1358 of SEQ ID NO:83) encodes a 420 amino acid protein (SEQ ID NO:84).

In one embodiment of a nucleotide sequence of human TANGO 206 the nucleotide at position 281 is a guanine (G). In this embodiment, the amino acid at position 61 is glutamate (E). In another embodiment of a nucleotide sequence of human TANGO 206, the nucleotide at position 281 is a cytosine (C). In this embodiment, the amino acid at position 61 is aspartate (D). In another embodiment of a nucleotide sequence of human TANGO 206, the nucleotide at position 326 is a guanine (G). In this embodiment, the amino acid at position 76 is a glutamate (E). In another embodiment of a nucleotide sequence of human TANGO 206, the nucleotide at position 326 is a cytosine (C). In this embodiment, the amino acid at position 76 is aspartate (D). In another embodiment of a nucleotide sequence of human TANGO 206, the nucleotide at position 329 is an adenine (A). In this embodiment, the amino acid at position 77 is a glutamate (E). In another embodiment of a nucleotide sequence of human TANGO 206, the nucleotide at position 329 is a cytosine (C). In this embodiment, the amino acid at position 77 is aspartate (D).

The presence of a methionine residue at amino acid residue positions 282, 339, 358, 369, and 400 indicates that there can be alternative forms of human TANGO 206 of 139 amino acids, 82 amino acids, 63 amino acids, 52 amino acids, and 21 amino acids, respectively.

Another embodiment of the invention includes isolated nucleic acid molecules comprising a polynucleotide having a nucleotide sequence encoding the polypeptide having the human TANGO 206 polypeptide sequence, but lacking the N-terminal methionine residue. In this embodiment, the nucleotide sequence of human TANGO 206, nucleotides 102-1358 of SEQ ID NO:83, encodes the human TANGO 206 amino acid sequence comprising amino acids 2-420 of SEQ ID NO:84.

The signal peptide prediction program SIGNALP (Nielsen et al. (1997) Protein Engineering 10:1-6) predicted that human TANGO 206 includes a 29 amino acid signal peptide (amino acid 1 to about amino acid 29 of SEQ ID NO:84) preceding the mature human TANGO 206 protein (corresponding to about amino acid 30 to amino acid 420 of SEQ ID NO:84).

In another example, a TANGO 206 family member also includes one or more of the following domains: (1) an extracellular domain; (2) a transmembrane domain; and (3) a cytoplasmic domain.

In one embodiment, a TANGO 206 protein contains a signal sequence of about amino acids 1-29. In certain embodiments, a TANGO 206 family member has the amino acid sequence, and the signal sequence is located at amino acids 1 to 27, 1 to 28, 1 to 29, 1 to 30 or 1 to 31. In such embodiments of the invention, the domains and the mature protein resulting from cleavage of such signal peptides are also included herein. For example, the cleavage of a signal sequence consisting of amino acids 1 to 29 results in a mature TANGO 206 protein corresponding to amino acids 30 to 420 of SEQ ID NO:84. The signal sequence is normally cleaved during processing of the mature protein.

In one embodiment, a TANGO 206 protein contains an extracellular domain of about amino acids 30-362 of SEQ ID NO:84. In one embodiment, a TANGO 206 protein contains a transmembrane of about amino acids 363-379 of SEQ ID NO:84. In another embodiment, a TANGO 206 protein contains a cytoplasmic domain of about amino acids 380-386 of SEQ ID NO:84. In another embodiment, a TANGO 206 protein includes a transmembrane domain of about amino acids 387-405 of SEQ ID NO:84. In still another embodiment, a TANGO 206 protein includes an extracellular domain of about amino acids 406-420 of SEQ ID NO:84.

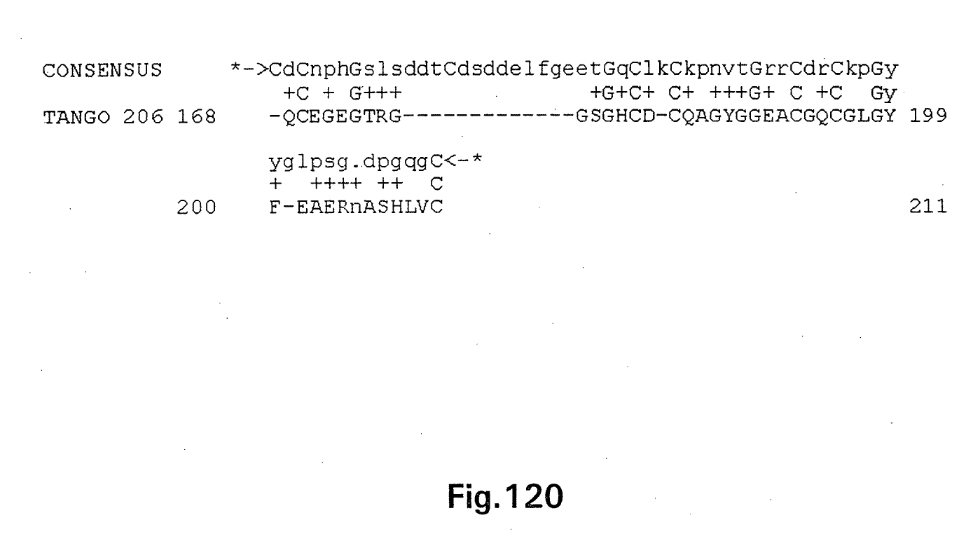

TANGO 206 family members can include a laminin EGF-like domain. A consensus laminin EGF-like domain has the sequence shown in the alignment depicted in FIG. 120, where the more conserved residues in the consensus sequence are indicated by uppercase letters and the less conserved residues in the consensus sequence are indicated by lowercase letters. The laminin EGF-like domain of human TANGO 204 is located at amino acids 168-211 of SEQ ID NO:84. Laminin EGF-like domains are similar to EGF domains except that they include eight cysteines rather than 6 cysteines. All eight cysteines are expected to participate in disulfide bonds.

Human TANGO 206 is a transmembrane protein having a first extracellular domain which extends from about amino acid 30 to about amino acid 362, a first transmembrane domain which extends from about amino acid 363 to about amino acid 379, a cytoplasmic domain which extends from about amino acid 380 to about amino acid 386, a second transmembrane domain which extends from about amino acid 387 to about amino acid 405, and a second extracellular domain which extends from about amino acid 406 to amino acid 420 of SEQ ID NO:84.

Alternatively, in another embodiment, a human TANGO 206 is a transmembrane protein having a first cytoplasmic domain which extends from about amino acid 30 to about amino acid 362, a first transmembrane domain which extends from about amino acid 363 to about amino acid 379, an extracellular domain which extends from about amino acid 380 to about amino acid 386, a second transmembrane domain which extends from about amino acid 387 to about amino acid 405, and a second cytoplasmic domain which extends from about amino acid 406 to amino acid 420 of SEQ ID NO:84.

Human TANGO 206 includes a laminin EGF-like domain at amino acids 168-211 of SEQ ID NO:84. FIG. 110A-110E depicts an alignment of the laminin EGF-like domain of human TANGO 206 with a laminin EGF-like domain derived from a hidden Markov model.

Human TANGO 206 that has not been post-translationally modified is predicted to have a molecular weight of 45.4 kDa prior to cleavage of its signal peptide and a molecular weight of 42.1 kDa subsequent to cleavage of its signal peptide.

N-glycosylation sites are present at amino acids 79-82 and 205-208. A cAMP and cGMP-dependent protein kinase phosphorylation site is present at amino acids 290-293. Protein kinase C phosphorylation sites are present at amino acids 48-50, 63-65, 138-140, 159-161, 406-408, and 409-411. Casein kinase II phosphorylation sites are present at amino acids 63-66, 73-76, 99-102, 222-225, and 359-362. N-myristoylation sites are present at amino acids 8-13, 51-56, 59-64, 69-74, 167-172, 173-178, 188-193, 250-255, 267-272, 280-285, 326-331, 372-377, and 395-400. An aspartic acid and asparagine hydroxylation site is present at amino acids 321-332. An EGF-like domain cysteine pattern signature is present at amino acids 181-192.

Clone Athoc49b12, which encodes human TANGO 206, was deposited as EpT206 with the American Type Culture Collection (10801 University Boulevard, Manassas, Va. 20110-2209) on Apr. 21, 1999 and assigned Accession Number 207223. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

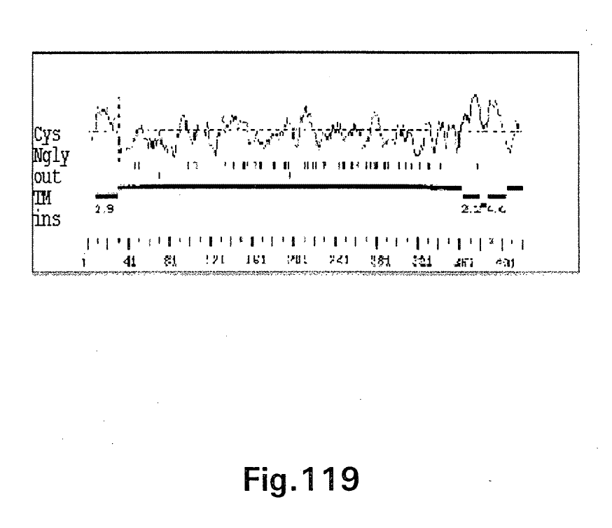

FIG. 119 depicts a hydropathy plot of human TANGO 206. The hydropathy plot indicates the presence of a signal sequence at the amino-terminus of human TANGO 206 and two transmembrane domains within human TANGO 206, suggesting that human TANGO 206 is a transmembrane protein.

Northern analysis of human TANGO 206 mRNA expression revealed strong expression in the heart, moderate expression in the skeletal muscle and weak expression in the kidney, brain, and placenta.

The human TANGO 206 gene maps to chromosome 3 between D3S3591 and D3S1283 based on the homology between a portion of human TANGO 206 and Genbank Accession Number G06979 (human STS WI-8719), which is reported to map to this position.

### Mouse TANGO 206

A cDNA encoding mouse TANGO 206 was identified by analyzing the sequences of clones present in a mouse bone marrow cDNA library.

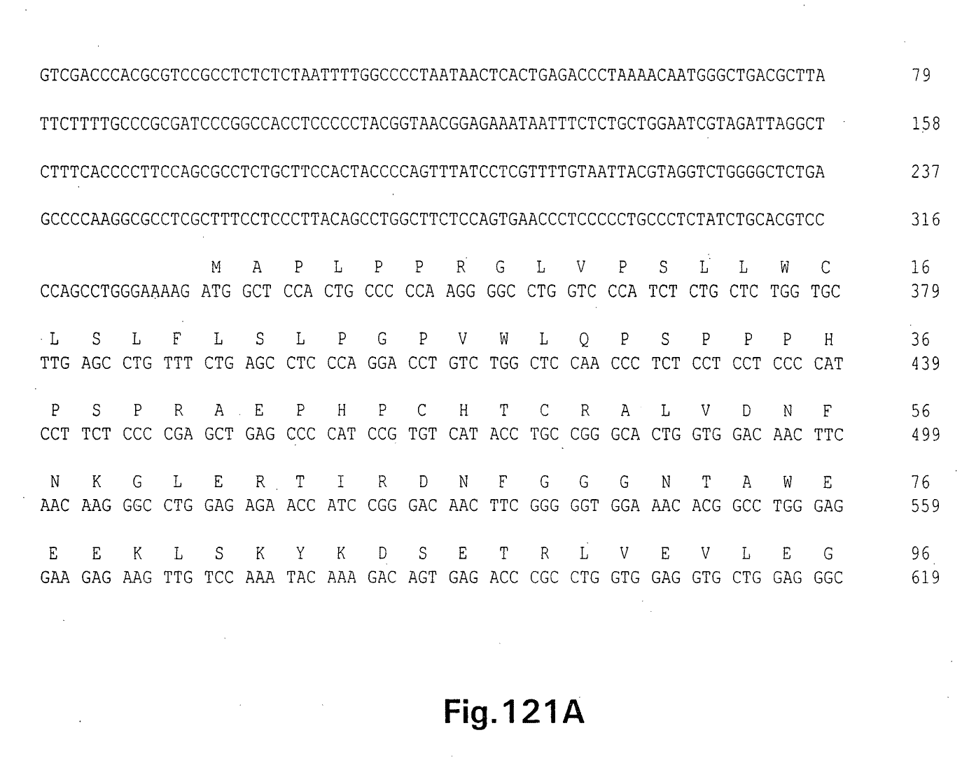

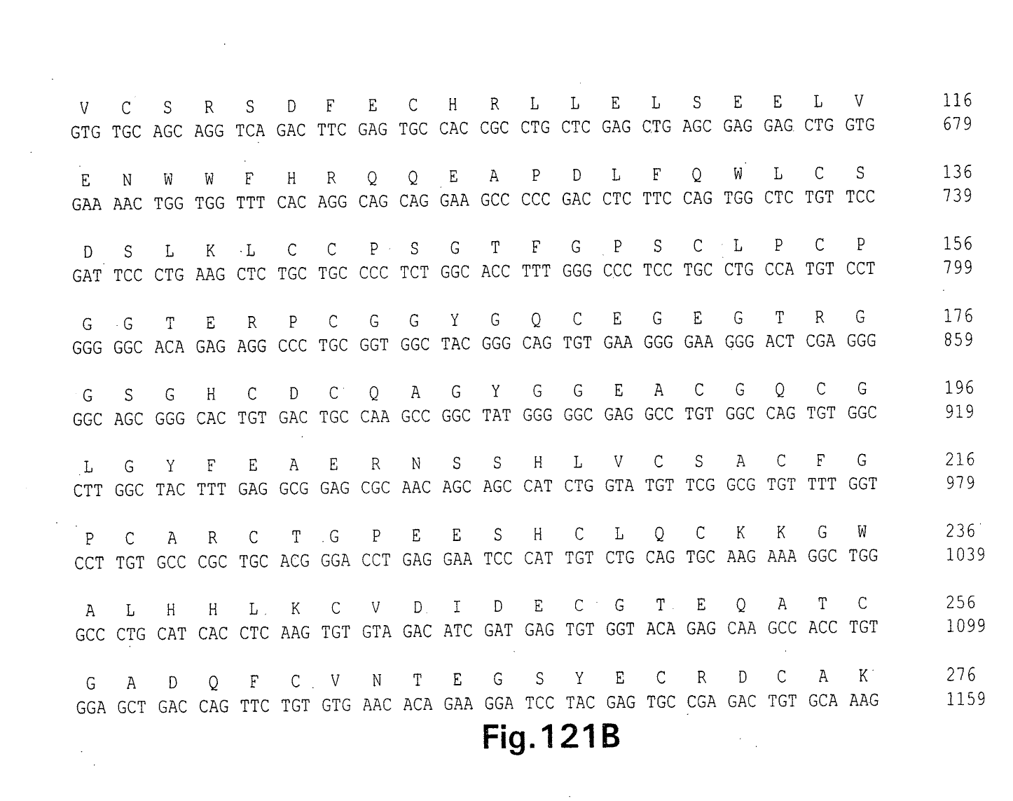

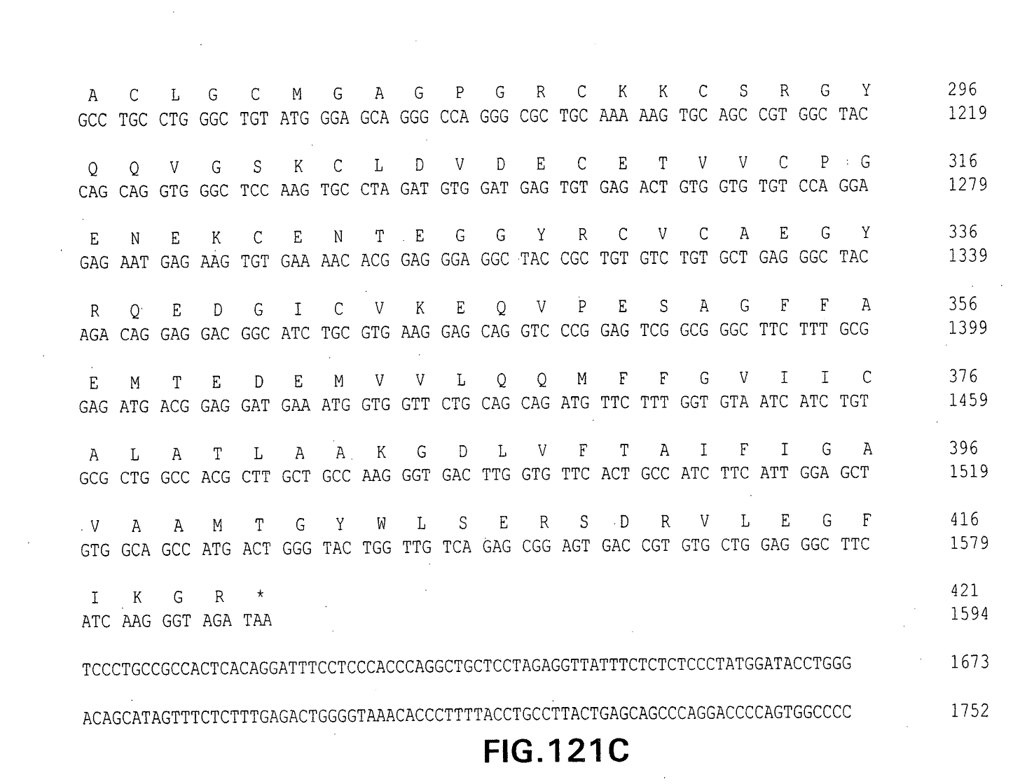

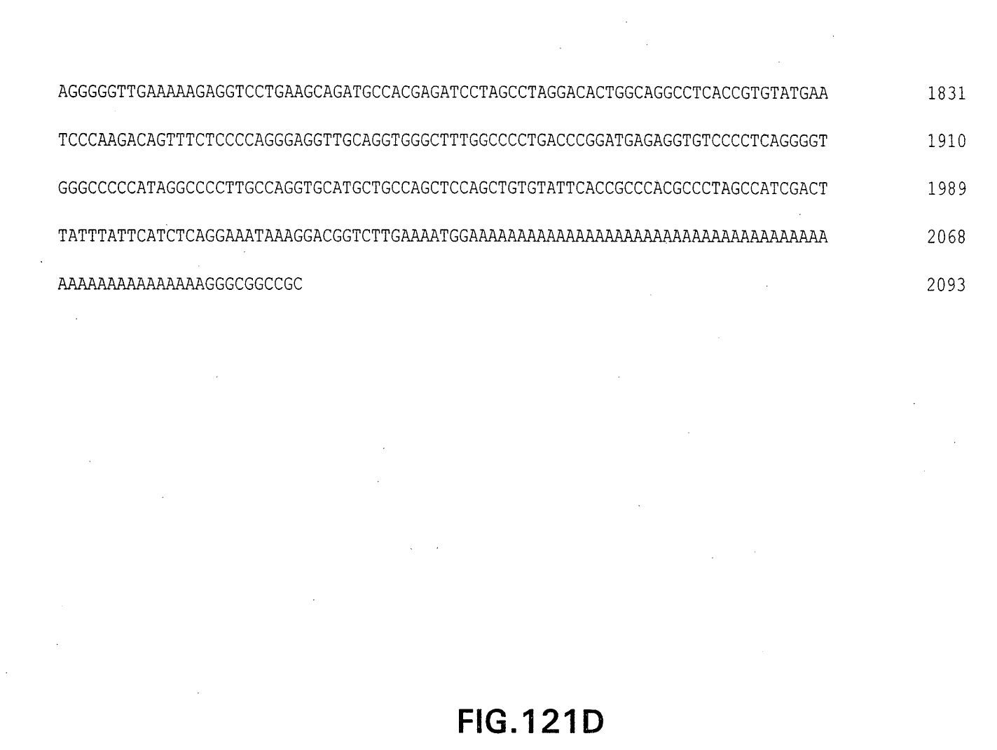

This analysis led to the identification of a clone, AtmMa206, encoding full-length mouse TANGO 206. The cDNA of this clone is 2093 nucleotides long (FIG. 121A-121D; SEQ ID NO:85). The 1260 nucleotide open reading frame of this cDNA (nucleotides 332-1591 of SEQ ID NO:85) encodes a 420 amino acid protein (SEQ ID NO:86).

In one embodiment of a nucleotide sequence of mouse TANGO 206, the nucleotide at position 457 is a guanine (G). In this embodiment, the amino acid at position 42 is glutamate (E). In another embodiment of a nucleotide sequence of mouse TANGO 206, the nucleotide at position 457 is a cytosine (C). In this embodiment, the amino acid at position 42 is aspartate (D). In another embodiment of a nucleotide sequence of mouse TANGO 206, the nucleotide at position 514 is a guanine (G). In this embodiment, the amino acid at position 61 is a glutamate (E). In another embodiment of a nucleotide sequence of mouse TANGO 206, the nucleotide at position 514 is a cytosine (C). In this embodiment, the amino acid at position 61 is aspartate (D). In another embodiment of a nucleotide sequence of mouse TANGO 206, the nucleotide at position 559 is an adenine (A). In this embodiment, the amino acid at position 76 is a glutamate (E). In another embodiment of a nucleotide sequence of mouse TANGO 206, the nucleotide at position 559 is a cytosine (C). In this embodiment, the amino acid at position 76 is aspartate (D).

The presence of a methionine residue at positions 282, 358, 363, 369, and 400 indicates that there can be alternative forms of mouse TANGO 206 of 139 amino acids, 63 amino acids, 58 amino acids, 52 amino acids, and 21 amino acids, respectively.

Another embodiment of the invention includes isolated nucleic acid molecules comprising a polynucleotide having a nucleotide sequence encoding the polypeptide having the mouse TANGO 206 polypeptide sequence, but lacking the N-terminal methionine residue. In this embodiment, the nucleotide sequence of mouse TANGO 206, nucleotides 335-1591 of SEQ ID NO:85, encodes the mouse TANGO 206 amino acid sequence from amino acids 2-420 of SEQ ID NO:86.

The signal peptide prediction program SIGNALP (Nielsen et al (1997) Protein Engineering 10:1-6) predicted that mouse TANGO 206 includes a 29 amino acid signal peptide (amino acid 1 to about amino acid 29 of SEQ ID NO:86) preceding the mature mouse TANGO 206 protein (corresponding to about amino acid 30 to amino acid 420 of SEQ ID NO:86).

Mouse TANGO 206 is a transmembrane protein having a first extracellular domain which extends from about amino acid 30 to about amino acid 362, a first transmembrane domain which extends from about amino acid 363 to about amino acid 379, a cytoplasmic domain which extends from about amino acid 380 to about amino acid 386, a second transmembrane domain which extends from about amino acid 387 to about amino acid 405, and a second extracellular domain which extends from about amino acid 406 to about amino acid 420 of SEQ ID NO:86.

Alterantively, mouse TANGO 206 is a transmembrane protein having a first cytoplasmic domain which extends from about amino acid 30 to about amino acid 362, a first transmembrane domain which extends from about amino acid 363 to about amino acid 379 an extracellular domain which extends from about amino acid 380 to about amino acid 386, a second transmembrane domain which extends from about amino acid 387 to about amino acid 405, and a second cytoplasmic domain which extends from about amino acid 406 to about amino acid 420 of SEQ ID NO:86.

Mouse TANGO 206 that has not been post-translationally modified is predicted to have a molecular weight of 45.7 kDa prior to cleavage of its signal peptide and a molecular weight of 42.4 kDa subsequent to cleavage of its signal peptide.

Mouse TANGO 206 includes a laminin EGF-like domain at amino acids 168-211 and two EGF-like domains, one at amino acids 155-192 and one at amino acids 309-343.

In situ tissue screening was performed on mouse adult and embryonic tissue to analyze the expression of mouse TANGO 206 mRNA. In summary, expression during embryogenesis was observed ubiquitously in the central nervous system of the ages examined. It was also observed in the eye and the large ganglion of the head. Expression was also observed in the liver from E13.5 to E15.5. Expression pattern was multifocal in a pattern suggestive of megakaryocytes or haemopoietic islands. Expression was also observed in the skin of the earlier embryonic ages. Adult expression was observed ubiquitously in the brain and grey matter of the spinal cord. The adrenal gland and small intestine also had moderate to strong expression.

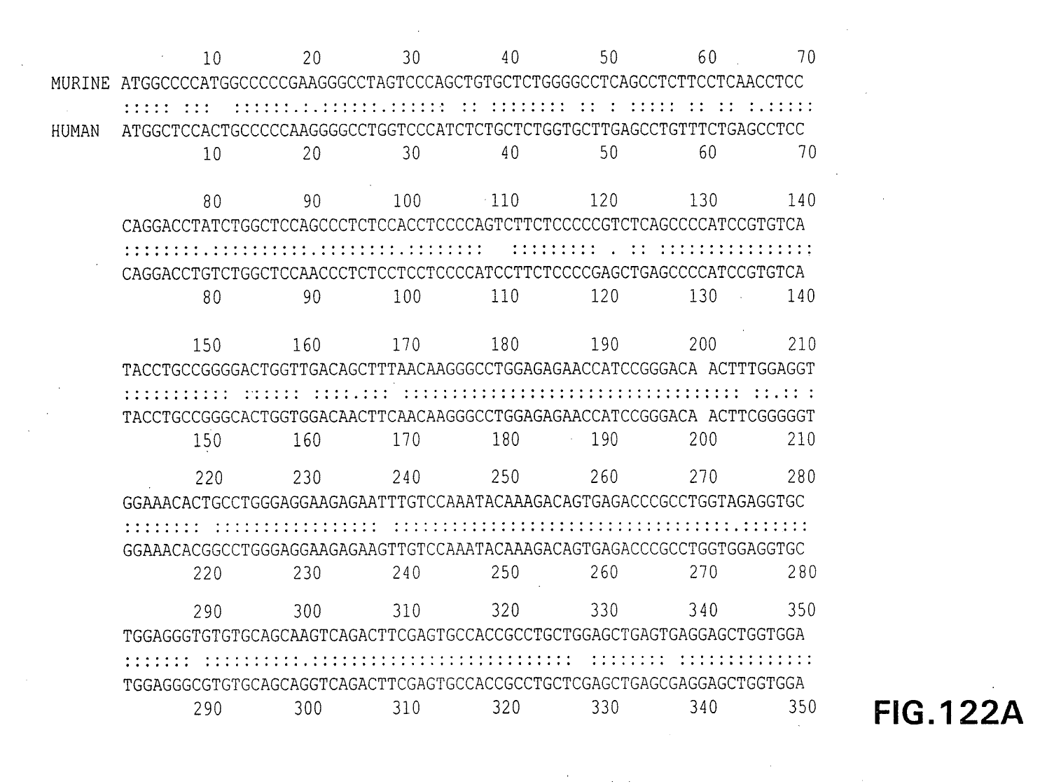

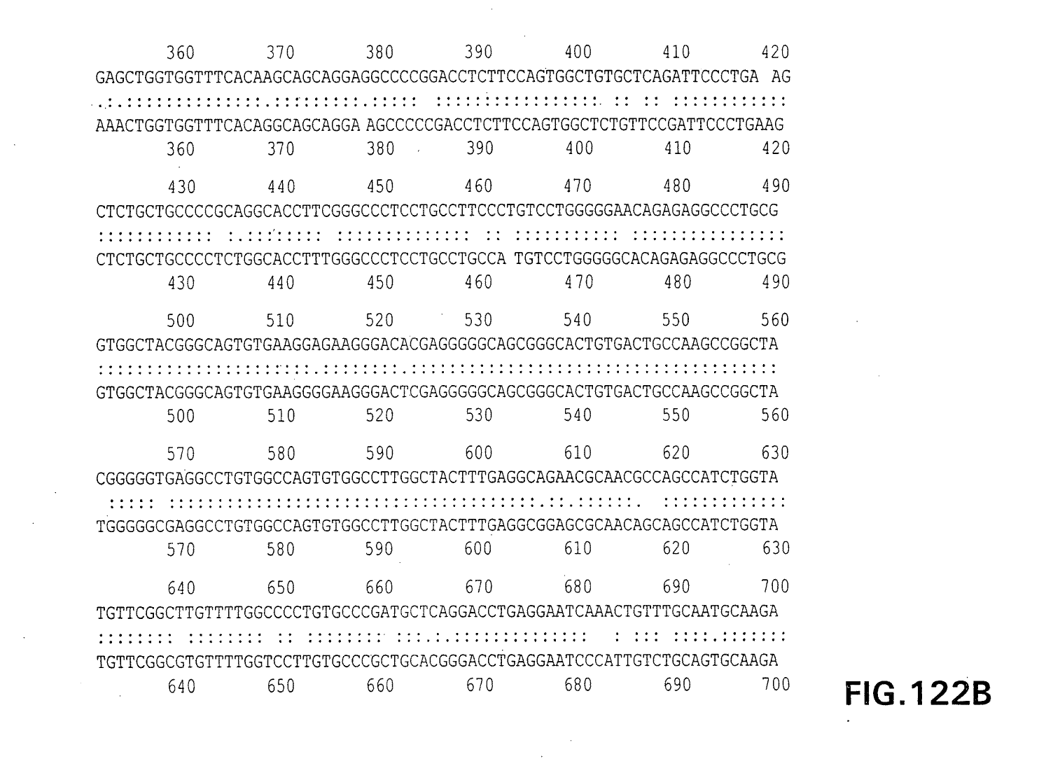

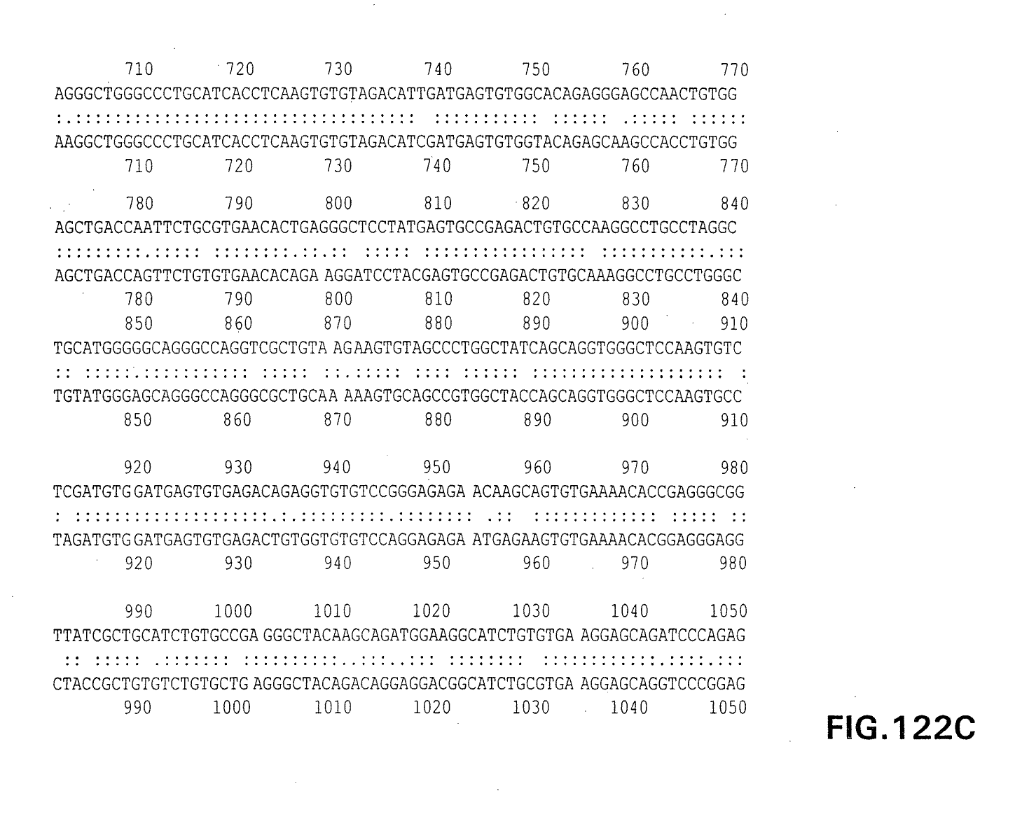

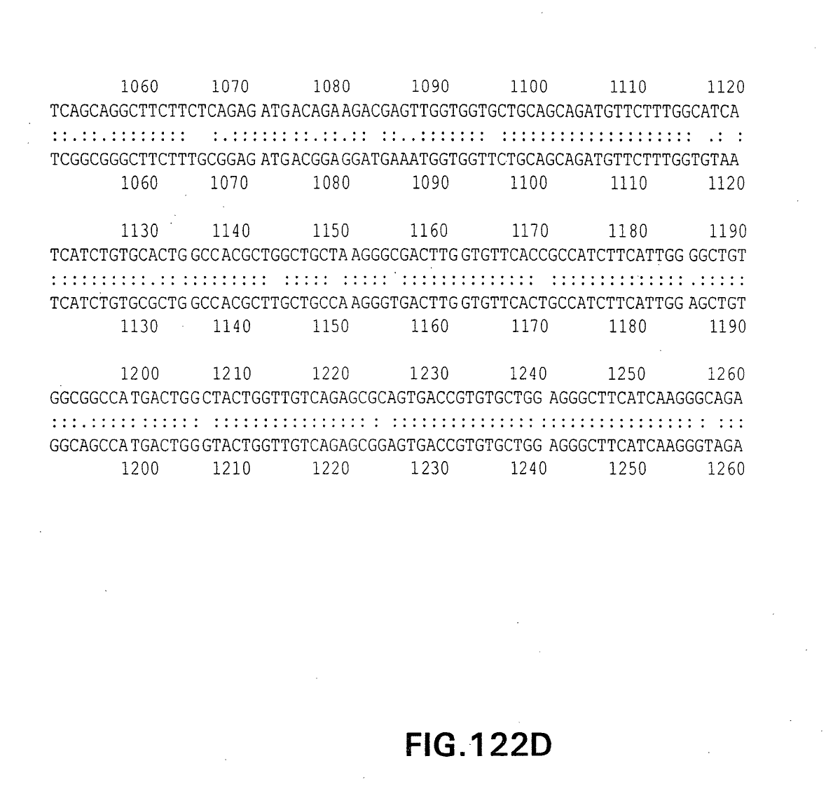

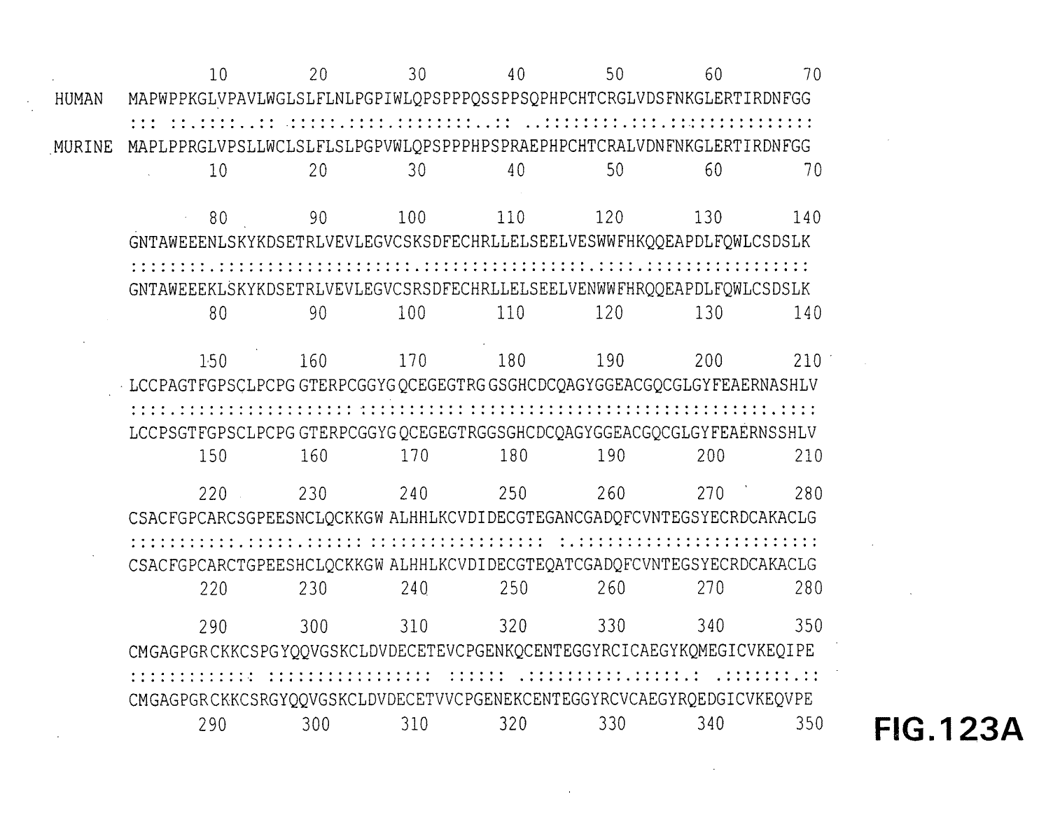

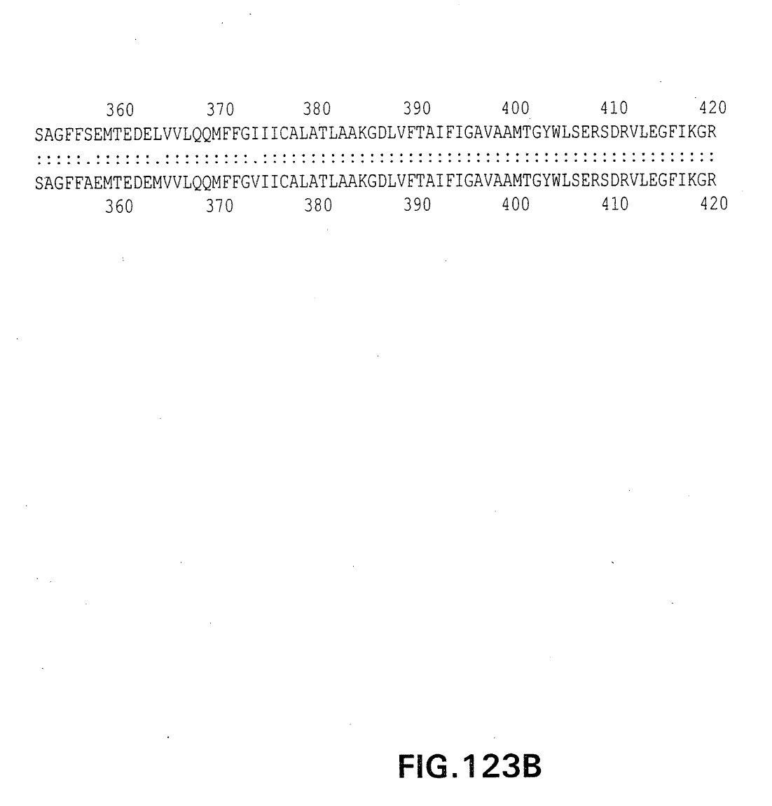

Human and mouse TANGO 206 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 91.4%. The human and mouse TANGO 206 full length cDNAs are 84% identical, as assessed using the same software and parameters as indicated. In the respective ORFs, calculated in the same fashion as the full length cDNAs, human and mouse TANGO 206 are 89% identical. The nucleotide sequence and amino acid sequence alignments of human and mouse TANGO 206 can be found in FIG. 122A-122D and FIG. 123A-123B, respectively.

Clone AtmMa206, which encodes mouse TANGO 206, was deposited as EpTm206 with the American Type Culture Collection (10801 University Boulevard, Manassas, Va. 20110-2209) on Apr. 21, 1999 and assigned Accession Number 207221. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Use of TANGO 206 Nucleic Acids, Polypeptides, and Modulators Thereof

TANGO 206 polypeptides, nucleic acids, and modulators thereof, can be used to modulate the function, morphology, proliferation and/or differentiation of cells in the tissues in which it is expressed. TANGO 206 includes an laminin EGF domain and an EGF-like domain. Proteins having such domains play a role in a wide variety of biological processes, including cholesterol uptake, blood coagulation, specification of cell fate. TANGO 206 polypeptides, nucleic acids, and modulators thereof can be used to modulate cell proliferation, morphogenesis, tissue repair and renewal, terminal differentiation, cell survival, and cell migration. They can be used to treat cancer, promote would healing (e.g., of the skin, cornea, or digestive mucosa), treat familia hypercholesterolemia, treat hemophilia B, treat Marfan syndrome, and treat protein S deficiency, and modulate an allergic or inflammatory response. TANGO 206 polypeptides, nucleic acids, and modulators thereof can be used to modulate acid secretion, modulate tropic effects on gastrointestinal mucosa, modulate mucosal adaptation, and modulate gastroduodenal cell migration and proliferation. Thus, such molecules can be used to protect gastric mucosa against injury and promote gastroduodenal ulcer healing.

As human TANGO 206 was originally found in a LPS stimulated human primary osteoblast library, TANGO 206 nucleic acids, proteins, and modulators thereof can be used to modulate the proliferation, differentiation, and/or function of cells that form bone matrix, e.g., osteoblasts and osteoclasts, and can be used to modulate the formation of bone matrix. Thus A259 nucleic acids, proteins, and modulators thereof can be used to treat cartilage and bone associated diseases and disorders, and can play a role in bone growth, formation, and remodeling. Examples of cartilage and bone associated diseases and disorders include e.g., bone cancer, achondroplasia, myeloma, fibrous dysplasia, scoliosis, osteoarthritis, osteosarcoma, and osteoporosis.

As mouse TANGO 206 was originally found in a bone marrow library, TANGO 206 nucleic acids, proteins, and modulators thereof can be used to modulate the proliferation, differentiation, and/or function of cells that appear in the bone marrow, e.g., stem cells (e.g., hematopoietic stein cells), and blood cells, e.g., erythrocytes, platelets, and leukocytes. Thus A259 nucleic acids, proteins, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

TANGO 206 polypeptides, nucleic acids, and modulators thereof, can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which TANGO 206 is expressed include, for example, heart, brain, skeletal muscle, placenta, CNS, liver, small intestine, adrenal gland, and the kidney.

In another example, TANGO 206 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 206 polypeptides, nucleic acids, or modulators thereof, can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

As TANGO 206 exhibits expression in the heart, TANGO 206 nucleic acids, proteins, and modulators thereof can be used to treat cardiovascular disorders as described herein.

In another example, TANGO 206 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g. alcoholic cirrhosis, biliary cirrhosis, and hemochromatosis), or malignant tumors (e.g., primary carcinoma, hepatoblastoma, and angiosarcoma).

In another example, TANGO 206 polypeptides, nucleic acids, or modulators thereof, can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 206 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, TANGO 206 polypeptides, nucleic acids, or modulators thereof, can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

### Human TANGO 209

A cDNA encoding human TANGO 209 was identified by analyzing the sequences of clones present in a human osteoblast cDNA library.

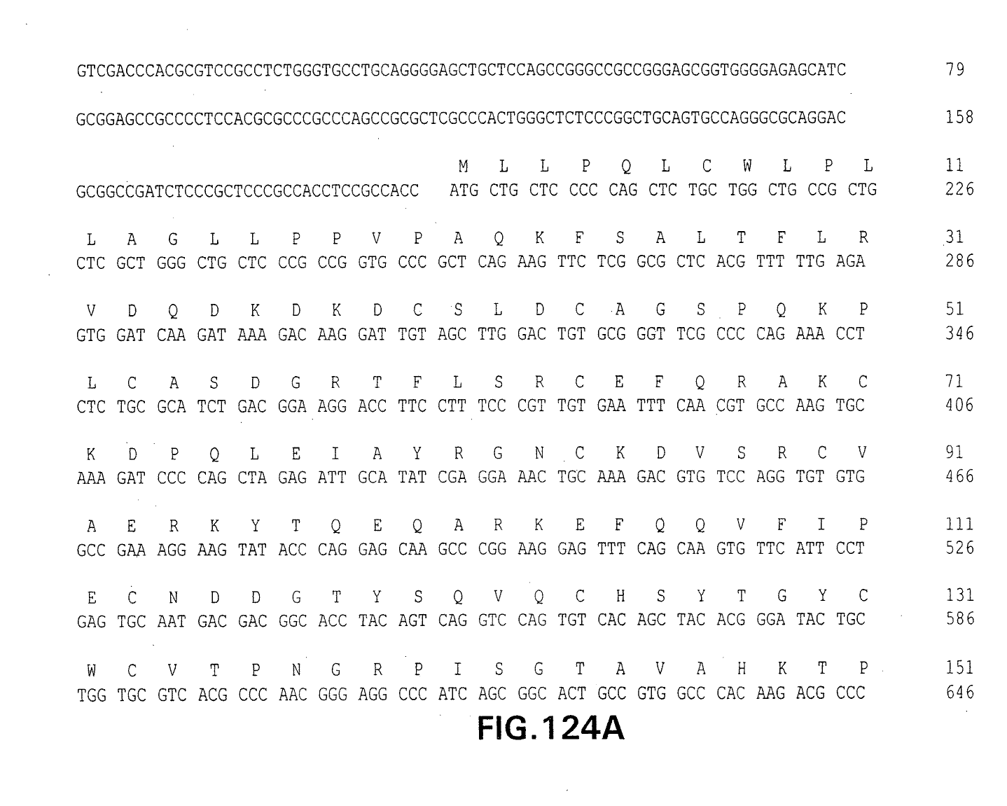

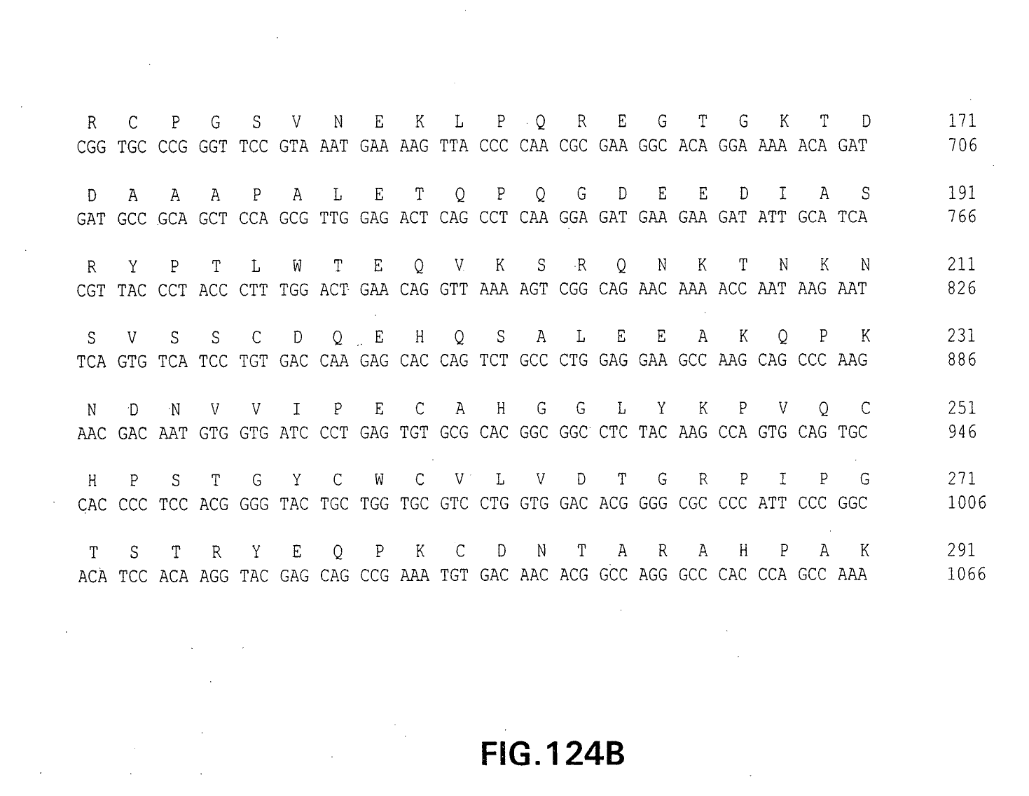

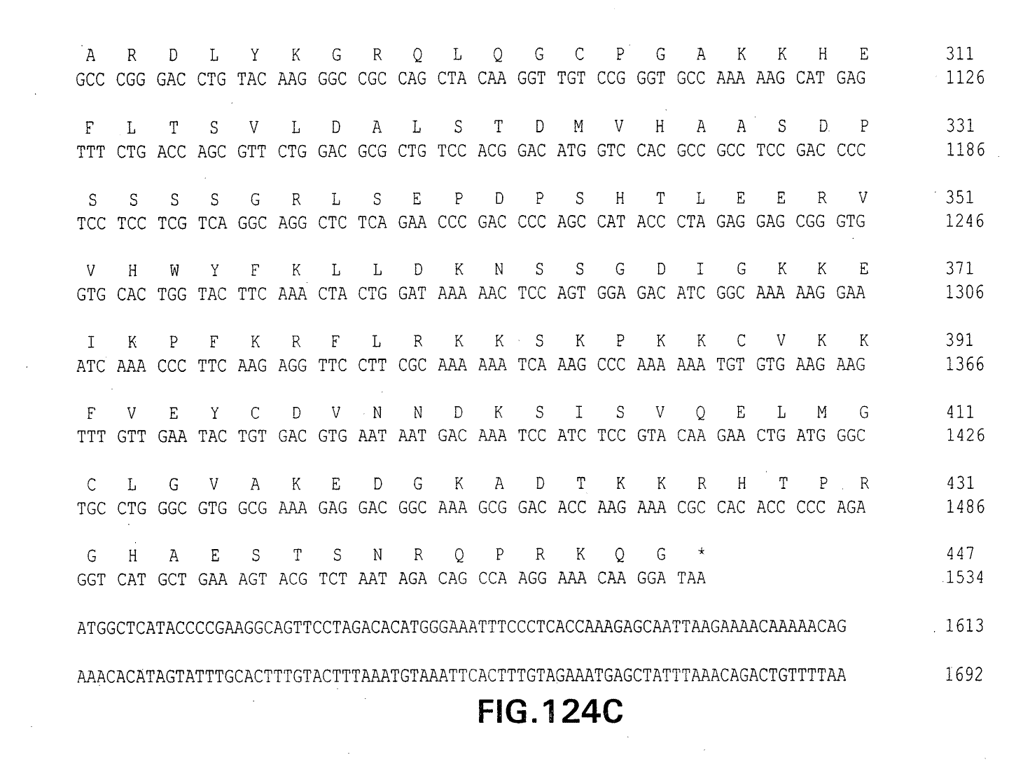

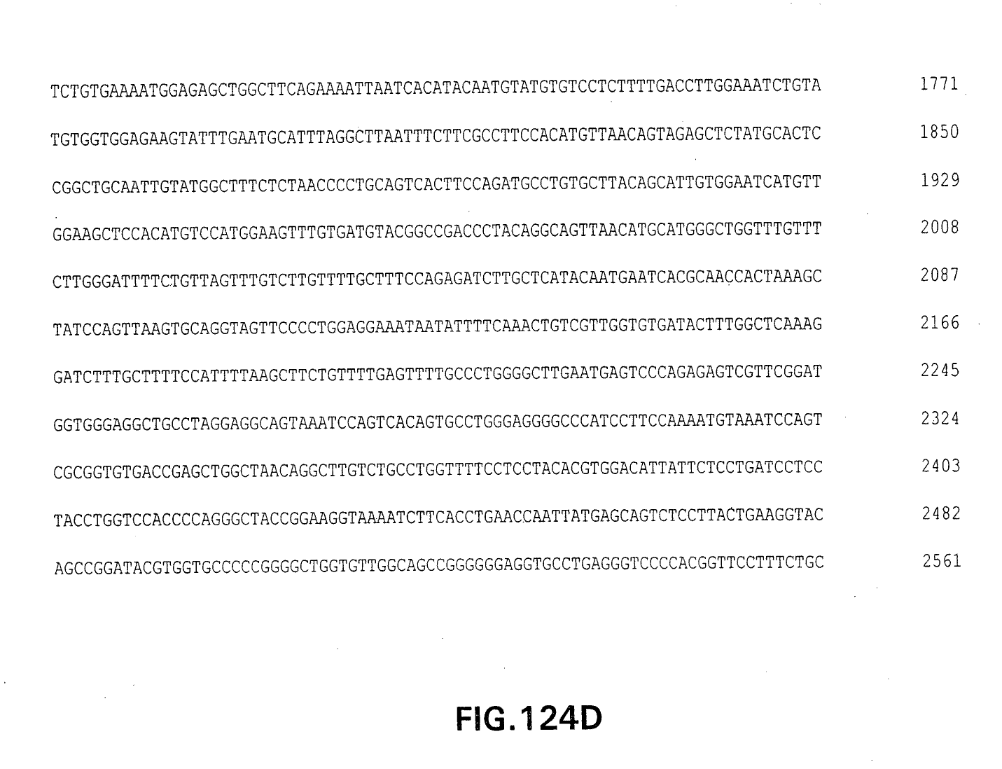

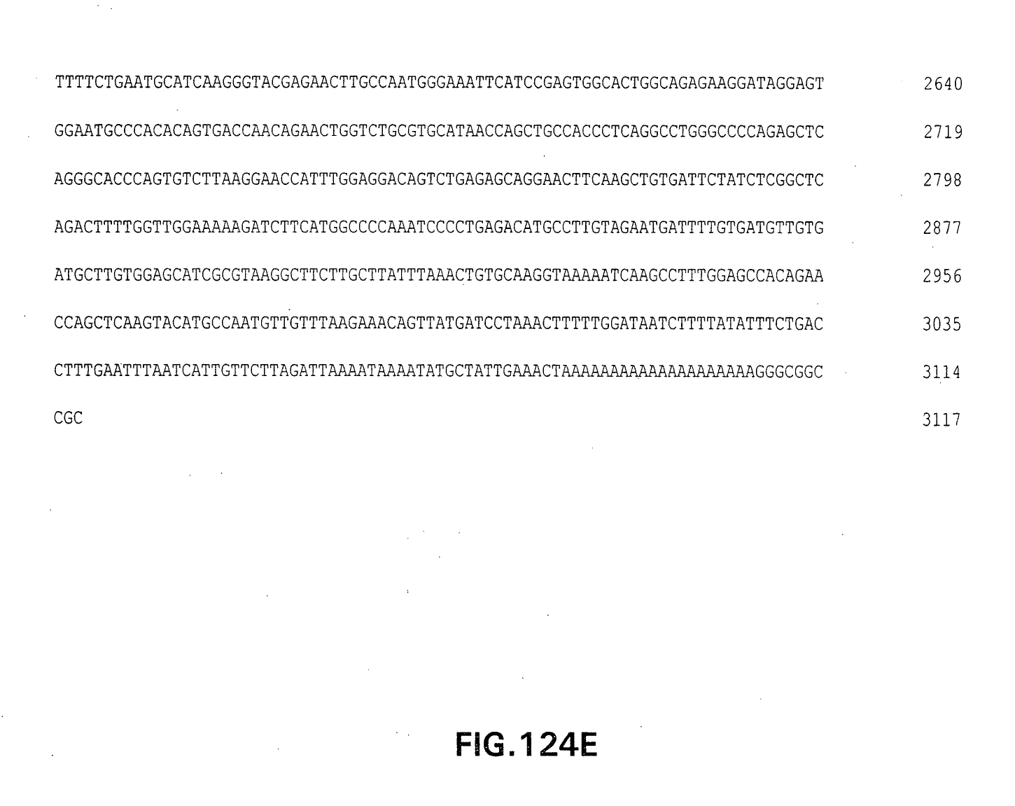

This analysis led to the identification of a clone, Athoc22d3, encoding full-length human TANGO 209. The cDNA of this clone is 3117 nucleotides long (FIG. 124A-124E; SEQ ID NO:87). The 1338 nucleotide open reading frame of this cDNA (nucleotides 194-1531 of SEQ ID NO:88) encodes a 446 amino acid protein (SEQ ID NO:88).

In one embodiment of a nucleotide sequence of human TANGO 209, the nucleotide at position 388 is an adenine (A). In this embodiment, the amino acid at position 65 is glutamate (E). In another embodiment of a nucleotide sequence of human TANGO 209, the nucleotide at position 388 is a cytosine (C). In this embodiment, the amino acid at position 65 is aspartate (D) In another embodiment of a nucleotide sequence of human TANGO 209, the nucleotide at position 424 is a guanine (G). In this embodiment, the amino acid at position 77 is a glutamate (E). In another embodiment of a nucleotide sequence of human TANGO 209, the nucleotide at position 424 is a cytosine (C). In this embodiment, the amino acid at position 77 is aspartate (D). In another embodiment of a nucleotide sequence of human TANGO 209, the nucleotide at position 472 is an adenine (A). In this embodiment, the amino acid at position 93 is a glutamate (E). In another embodiment of a nucleotide sequence of human TANGO 209, the nucleotide at position 472 is a cytosine (C). In this embodiment, the amino acid at position 93 is aspartate (D).

The presence of a methionine residue at positions 324, and 410 indicates that there can be alternative forms of human TANGO 209 of 123 amino acids, and 37 amino acids, respectively.

Another embodiment of the invention includes isolated nucleic acid molecules comprising a polynucleotide having a nucleotide sequence encoding the polypeptide having the human TANGO 209 amino acid sequence, but lacking the N-terminal methionine residue. In this embodiment, the nucleotide sequence of human TANGO 209, nucleotides 197-1531, encodes the human TANGO 209 amino acid sequence from amino acids 2-446 of SEQ ID NO:88.

The signal peptide prediction program SIGNALP (Nielsen et al. (1997) Protein Engineering 10:1-6) predicted that human TANGO 209 includes a 21 amino acid signal peptide (amino acid 1 to about amino acid 21 of SEQ ID NO:88) preceding the mature human TANGO 209 protein (corresponding to about amino acid 22 to amino acid 446 of SEQ ID NO:88).

Human TANGO 209 that has not been post-translationally modified is predicted to have a molecular weight of 49.7 kDa prior to cleavage of its signal peptide and a molecular weight of 47.3 kDa subsequent to cleavage of its signal peptide.

In one embodiment, a TANGO 209 protein contains a signal sequence of about amino acids 1-21. In certain embodiments, a TANGO 209 family member has the amino acid sequence, and the signal sequence is located at amino acids 1 to 19, 1 to 20, 1 to 21, 1 to 22 or 1 to 23. In such embodiments of the invention, the domains and the mature protein resulting from cleavage of such signal peptides are also included herein. For example, the cleavage of a signal sequence consisting of amino acids 1 to 21 results in a mature TANGO 209 protein corresponding to amino acids 22 to 446 of SEQ ID NO:88. The signal sequence is normally cleaved during processing of the mature protein.

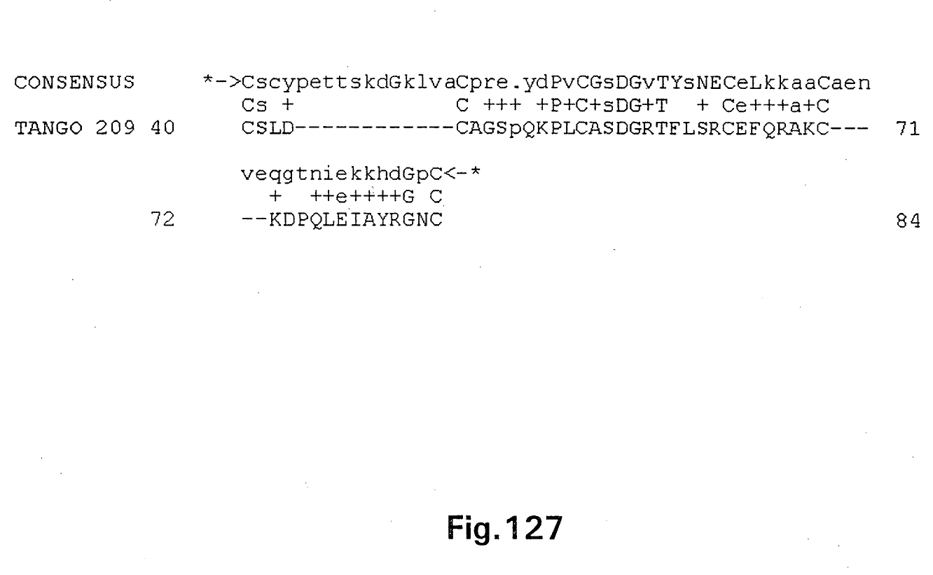

TANGO 209 family members can include a Kazal-type serine protease inhibitor domain. A consensus Kazal-type serine protease inhibitor domain has the sequence shown in the alignment depicted in FIG. 127, where the more conserved residues in the consensus sequence are indicated by uppercase letters and the less conserved residues in the consensus sequence are indicated by lowercase letters. The Kazal-type serine protease inhibitor domain of TANGO 209 is located at amino acids 40-84 of SEQ ID NO:88.

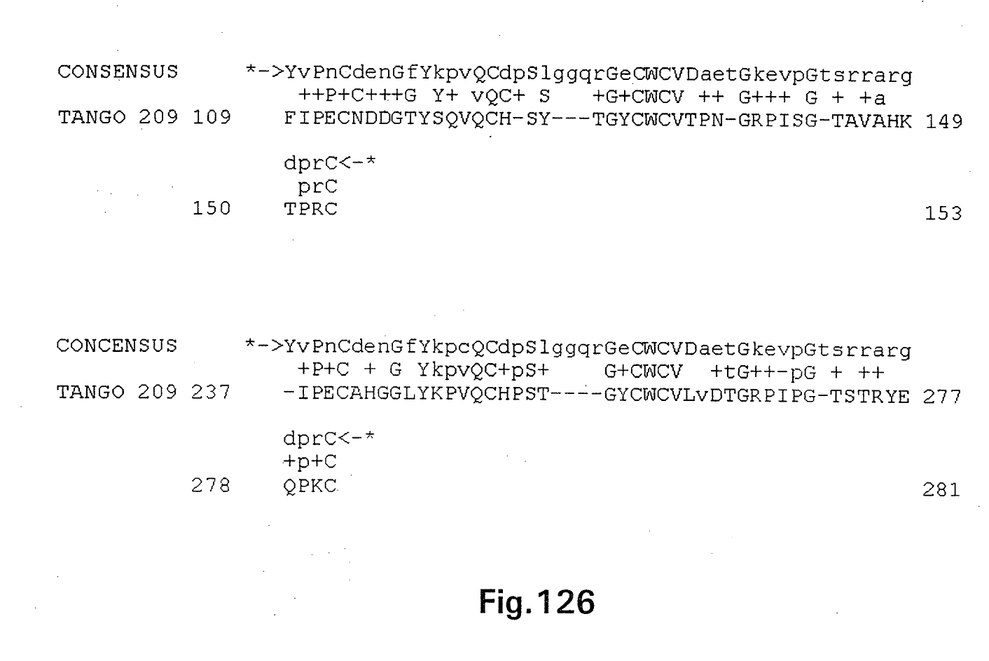

Human TANGO 209 includes thyroglobulin type 1 repeat domains at amino acids 109-153 and amino acids 237-281 of SEQ ID NO:88. FIG. 126 depicts an alignment of the thyroglobulin type 1 repeat domains of human TANGO 209 with a consensus thyroglobulin type 1 repeat domain derived from a hidden Markov model. Human TANGO 209 includes a Kazal-type serine protease inhibitor domain at amino acids 40-84 of SEQ ID NO:88. The thyroglobulin type 1 domain is present in HLA class II associate invariant chain, HLA class II associated invariant chain, and pancreatic carcinoma marker proteins GA733-1 and GA733-2.

FIG. 127 depicts an alignment of the Kazal-type serine protease inhibitor domain of human TANGO 209 with a consensus Kazal-type serine protease domain derived from a hidden Markov model.

N-glycosylation sites are present at amino acids 206-209 and 362-365. In human TANGO 209, cAMP and cGMP-dependent protein kinase phosphorylation sites are present at amino acids 94-97, 380-383, 426-429. Protein kinase C phosphorylation sites are present at amino acids 150-152, 167-169, 208-210, 265-267, 273-275, 284-286, 335-337, 424-426, 429-431, and 438-440. Casein kinase II phosphorylation sites are present at amino acids 62-65, 156-159, 214-217, 222-225, 274-277, 315-318, 339-342, 346-349, 363-366, and 405-408. A tyrosine kinase phosphorylation site is present at amino acids 89-96. N-myristoylation sites are present at amino acids 143-148, 166-171, and 303-308. An amidation site is present at amino acids 367-370. EF-hand calcium-binding domains are present at amino acids 360-372 and 397-409. A thyroglobulin type-1 repeat signature is present at amino acids 109-138.

Clone Athoc22d3, which encodes human TANGO 209, was deposited as EpT209 with the American Type Culture Collection (10801 University Boulevard, Manassas, Va. 20110-2209) on Apr. 21, 1999 and assigned Accession Number 207223. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

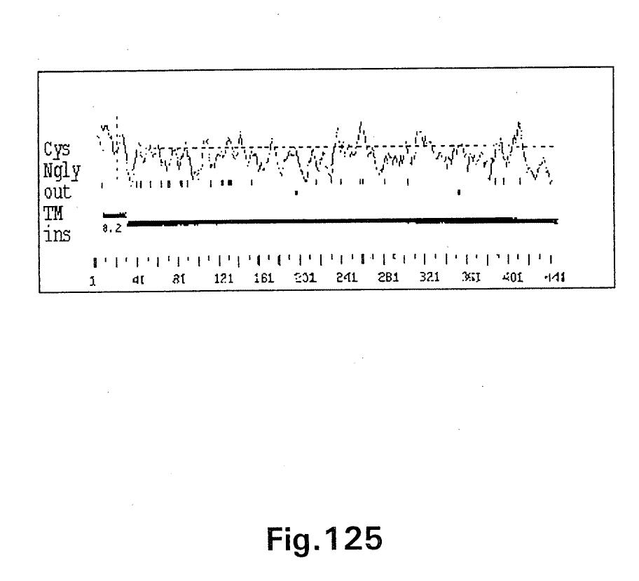

FIG. 125 depicts a hydropathy plot of human TANGO 209. The hydropathy plot indicates that human TANGO 209 has a signal sequence at its amino terminus, suggesting that human TANGO 209 is a secreted protein.

Northern analysis of human TANGO 209 mRNA expression revealed very high expression in the heart, high expression in the skeletal muscle and pancreas, and moderate expression in the placenta, lung and kidney.

The human gene for TANGO 209 was mapped on radiation hybrid panels to the long arm of chromosome 6, in the region q26-27. Flanking markers for this region are ATA22G07 and WI-9405. The MLLT4 (myeloid/lymphoid or mixed lineage leukemia) locus also maps to this region of the human chromosome. The PLG (plasminogen), VIP (vasoactive intestinal peptide), LPA (apolipoprotein Lp), MLLT4 (myeloid/lymphoid or mixed lineage leukemia), and THBS2 (thrombospondin 2) genes also map to this region of the human chromosome. This region is syntenic to mouse chromosome 17. The qk (quaking), T (brachyury), and het (head tilt) loci also map to this region of the mouse chromosome. The plg (plasminogen), qk (quaking), and het (head tilt) genes also map to this region of the mouse chromosome.

### Mouse TANGO 209

A cDNA encoding mouse TANGO 209 was identified by analyzing the sequences of clones present in a mouse osteoblast cDNA library.

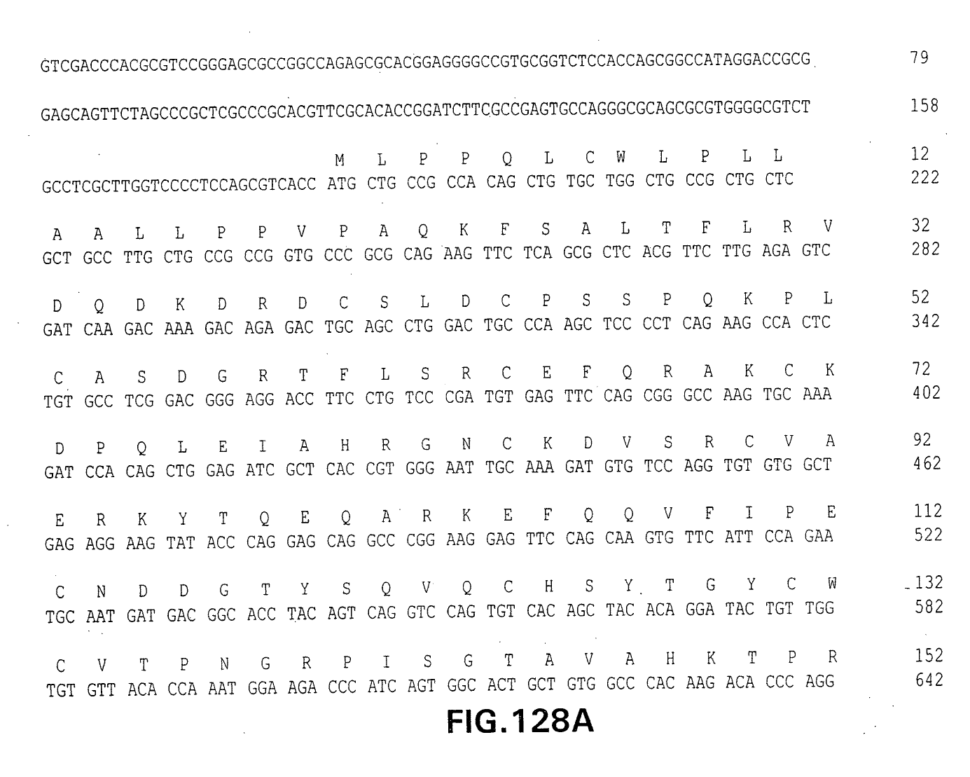

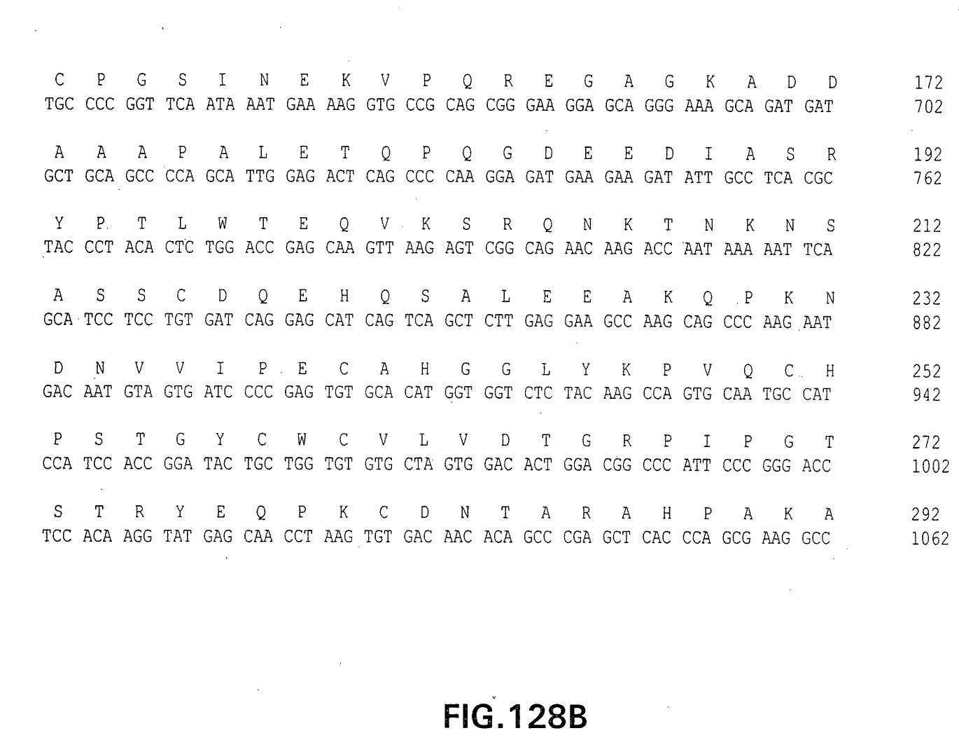

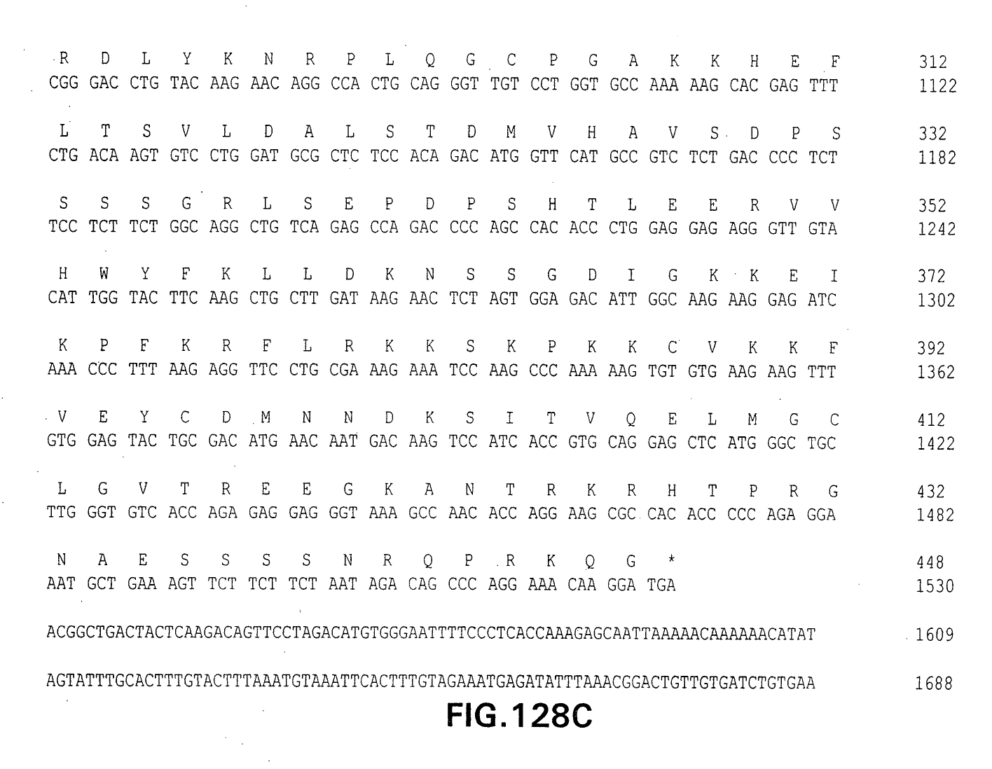

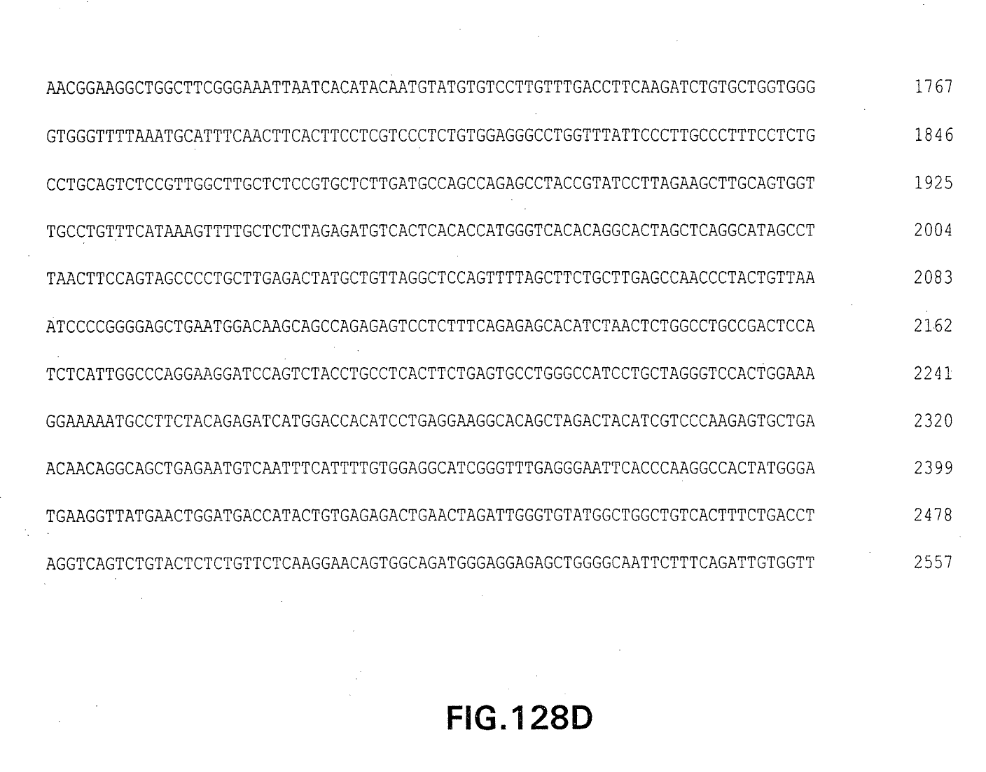

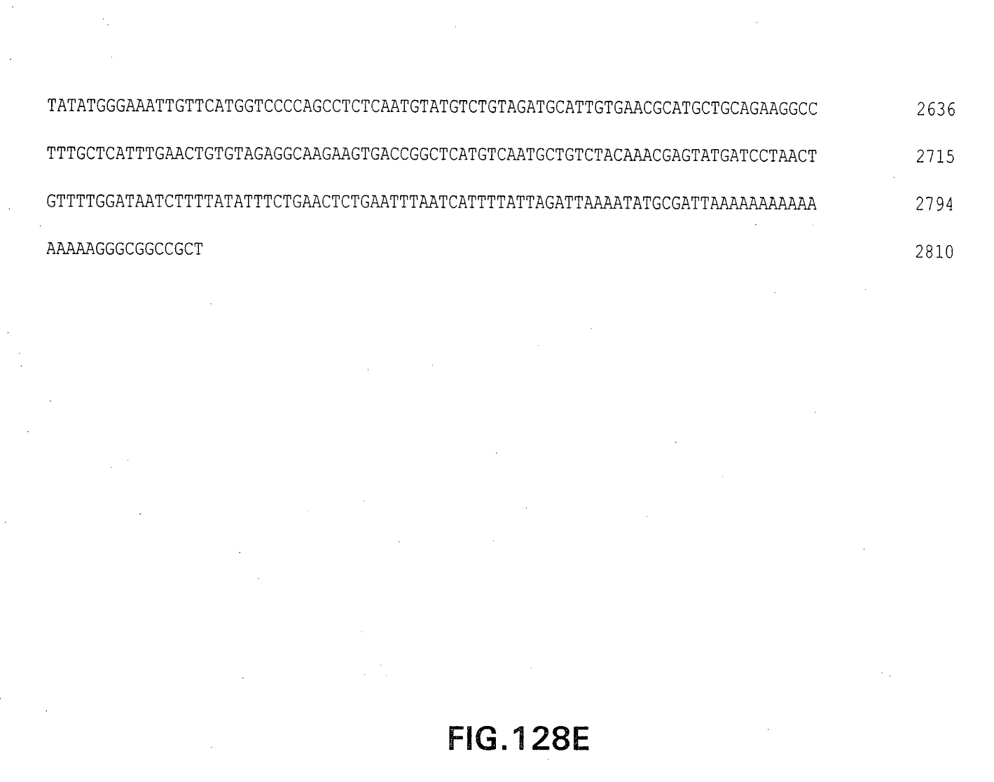

This analysis led to the identification of a clone, Atmoa99h11, encoding full-length mouse TANGO 209. The cDNA of this clone is 2810 nucleotides long (FIG. 128A-128E; SEQ ID NO:89). The 1341 nucleotide open reading frame of this cDNA (nucleotides 187 to 1527 of SEQ ID NO:89) encodes a 447 amino acid protein (SEQ ID NO:90).

In one embodiment of a nucleotide sequence of mouse TANGO 209 the nucleotide at position 381 is a guanine (G). In this embodiment, the amino acid at position 65 is glutamate (E). In another embodiment of a nucleotide sequence of mouse TANGO 209, the nucleotide at position 381 is a cytosine (C). In this embodiment, the amino acid at position 65 is aspartate (D). In another embodiment of a nucleotide sequence of mouse TANGO 209, the nucleotide at position 417 is an guanine (G). In this embodiment, the amino acid at position 77 is a glutamate (E). In another embodiment of a nucleotide sequence of mouse TANGO 209, the nucleotide at position 417 is a cytosine (C). In this embodiment, the amino acid at position 77 is aspartate (D). In another embodiment of a nucleotide sequence of mouse TANGO 209, the nucleotide at position 465 is a guanine (G). In this embodiment, the amino acid at position 93 is a glutamate (E). In another embodiment of a nucleotide sequence of mouse TANGO 209, the nucleotide at position 465 is a cytosine (C). In this embodiment, the amino acid at position 93 is aspartate (D).

The presence of a methionine residue at positions 324, and 398 indicate that there can be alternative forms of mouse TANGO 209 of 124 amino acids, and 50 amino acids, respectively.

Another embodiment of the invention includes isolated nucleic acid molecules comprising a polynucleotide having a nucleotide sequence encoding the polypeptide having the mouse TANGO 209 polypeptide sequence, but lacking the N-terminal methionine residue. In this embodiment, the nucleotide sequence of mouse TANGO 209, nucleotides 190 to 1527 of SEQ ID NO:89, encodes the mouse TANGO 209 amino acid sequence comprising amino acids 2-487 of SEQ ID NO:90.

The signal peptide prediction program SIGNALP (Nielsen et al. (1997) Protein Engineering 10:1-6) predicted that mouse TANGO 209 includes a 21 amino acid signal peptide (amino acid 1 to about amino acid 21 of SEQ ID NO:90) preceding the mature mouse TANGO 209 protein (corresponding to about amino acid 22 to amino acid 447 of SEQ ID NO:90).

Mouse TANGO 209 that has not been post-translationally modified is predicted to have a molecular weight of 49.9 kDa prior to cleavage of its signal peptide and a molecular weight of 47.5 kDa subsequent to cleavage of its signal peptide.

Mouse TANGO 209 includes thyroglobulin type 1 repeat domains at amino acids 109-153 and amino acids 237-281 of SEQ ID NO:90 and a Kazal-type serine protease inhibitor domain at amino acids 40-84 of SEQ ID NO:90.

In situ expression analysis of TANGO 209 expression in adult mice revealed expression in the brain (hippocampus, dentate gyrus, and frontal cortex), thymus (multifocal expression), kidney (medulla and capsule), and adrenal gland (capsule). Relatively high level, widespread, multifocal expression was observed in skeletal muscle. Multifocal expression was observed in the diaphragm. Relatively high level expression was observed in the spleen (non-follicular). Expression was observed in the bladder, where expression was highest in muscle tissue. Expression was observed in the small intestine and colon (smooth muscle, not villi). Expression was also observed in large vessels of the liver. High level, multifocal expression was observed in the heart.

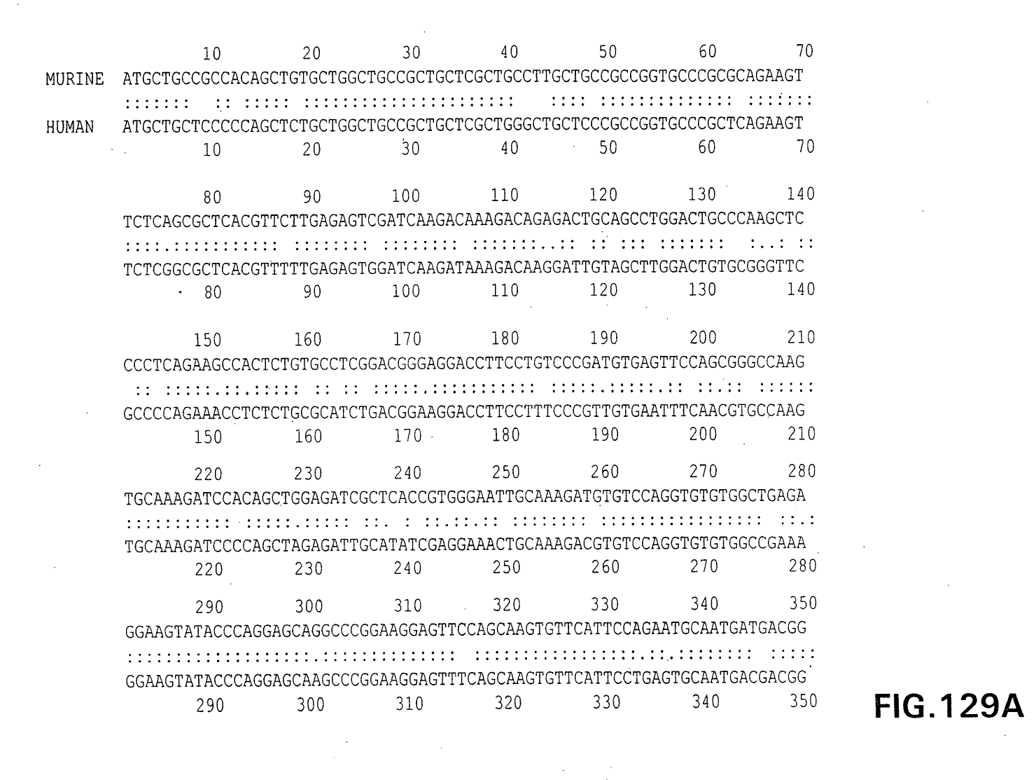

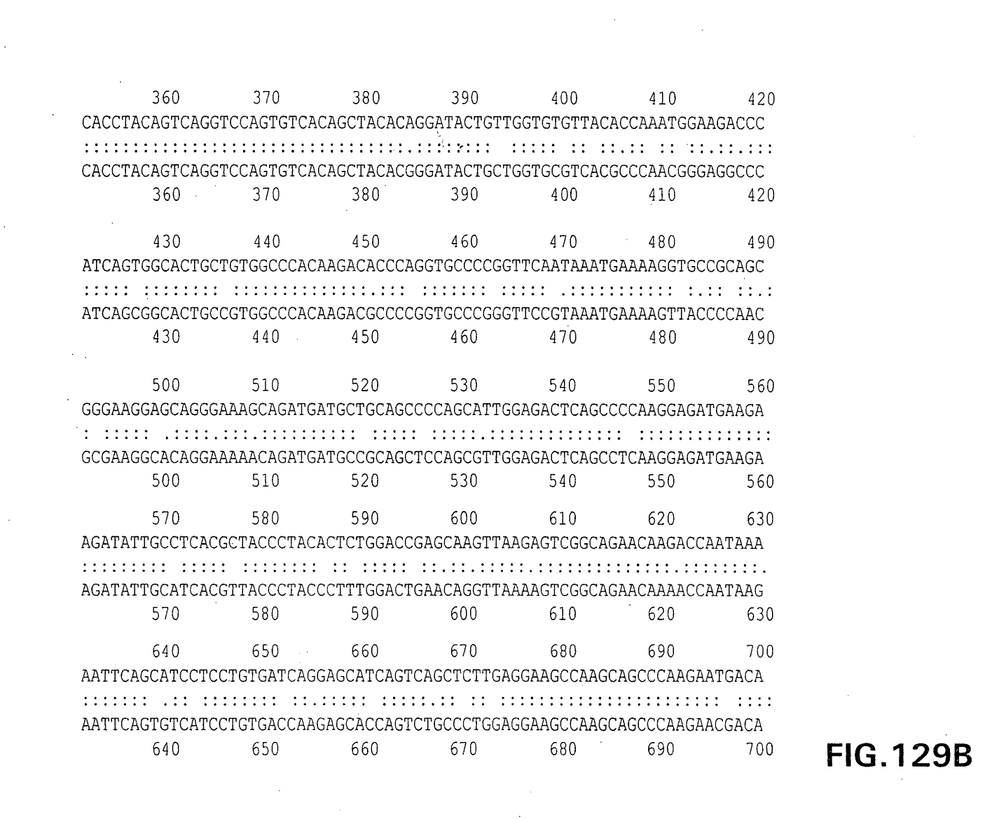

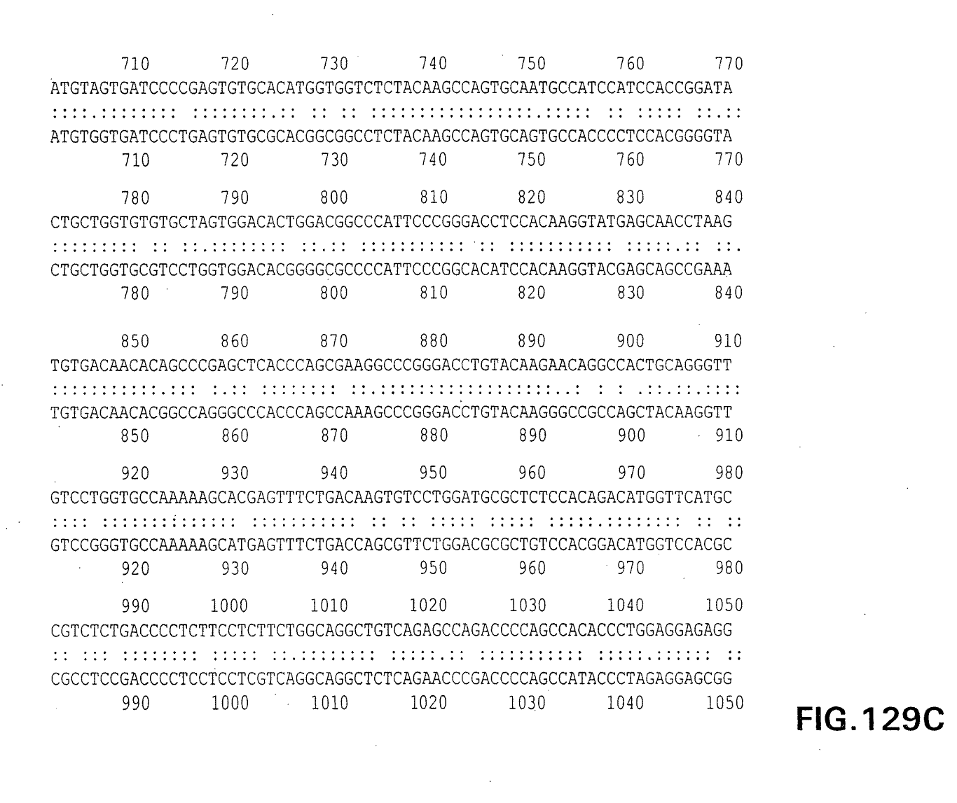

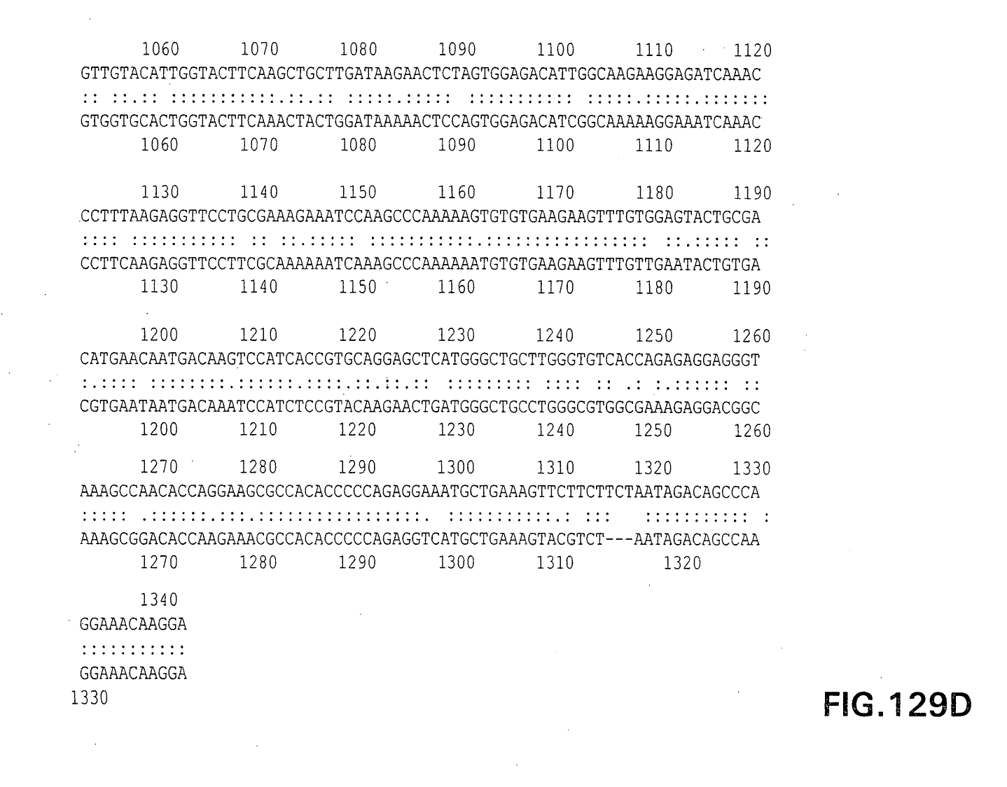

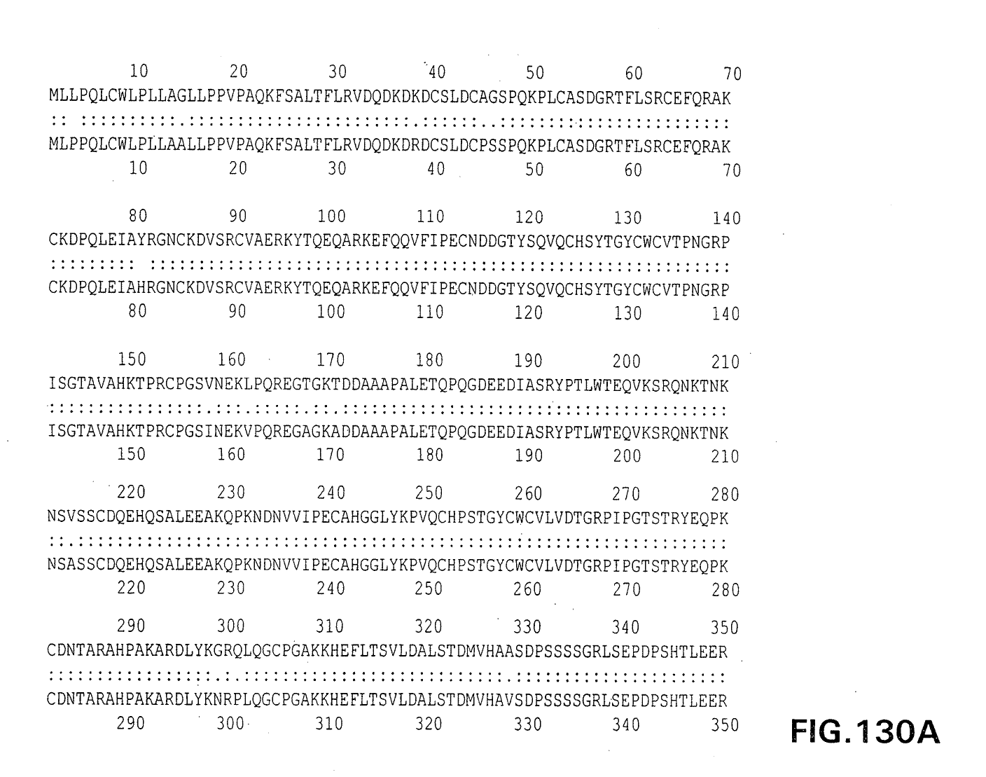

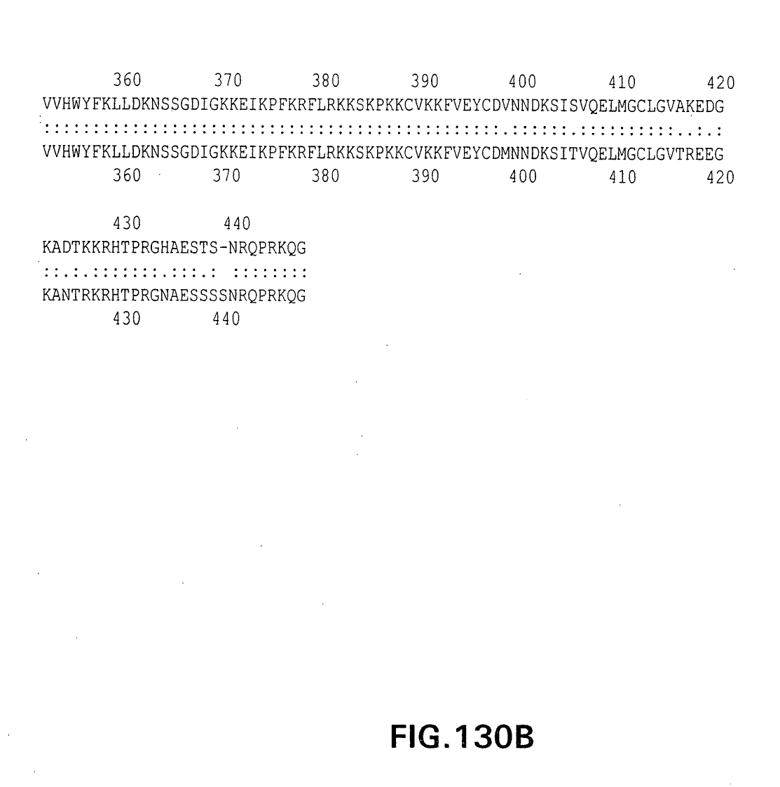

Human and mouse TANGO 209 sequences exhibit considerable similarity at the protein, nucleic acid, and open reading frame levels. An alignment (made using the ALIGN software (Myers and Miller (1989) CABIOS, ver. 2.0); BLOSUM 62 scoring matrix; gap penalties −12/−4), reveals a protein identity of 94.6%. The human and mouse TANGO 209 full length cDNAs are 77.7% identical, as assessed using the same software and parameters as indicated (without the BLOSUM 62 scoring matrix). In the respective ORFs, calculated in the same fashion as the full length cDNAs, human and mouse TANGO 209 are 84.4% identical. The nucleotide sequence and amino acid sequence alignments of human and mouse TANGO 209 can be found in FIG. 129A-129D and FIG. 130A-130B, respectively.

### Use of TANGO 209 Nucleic Acids, Polypeptides, and Modulators Thereof

TANGO 209 polypeptides, nucleic acids, and modulators thereof, can be used to modulate the function, morphology, proliferation and/or differentiation of cells in the tissues in which it is expressed. TANGO 209 polypeptides, nucleic acids, and modulators thereof can be used to treat disorders involving inappropriate activity of a serine protease and disorders of cellular migration, proliferation, and differentiation.

As human-TANGO 209 was originally found in a LPS stimulated human primary osteoblast library, TANGO 209 nucleic acids, proteins, and modulators thereof can be used to modulate the proliferation, differentiation, and/or function of cells that form bone matrix, e.g., osteoblasts and osteoclasts, and can be used to modulate the formation of bone matrix. Thus, TANGO 209 nucleic acids, proteins, and modulators thereof can be used to treat cartilage and bone associated diseases and disorders, and can play a role in bone growth, formation, and remodeling. Examples of cartilage and bone associated diseases and disorders include e.g., bone cancer, achondroplasia, myeloma, fibrous dysplasia, scoliosis, osteoarthritis, osteosarcoma, and osteoporosis.

TANGO 209 polypeptides, nucleic acids, and modulators thereof, can be used to modulate the function, morphology, proliferation and/or differentiation of cells in the tissues in which it is expressed. Such molecules can be used to treat disorders associated with abnormal or aberrant metabolism or function of cells in the tissues in which it is expressed. Tissues in which TANGO 209 is expressed include, for example, brain, skeletal muscle, thymus, liver, adrenal gland, and the kidney.

In another example, TANGO 209 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 209 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g. hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 209 polypeptides, nucleic acids, or modulators thereof, can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 209 polypeptides, nucleic acids, or modulators thereof, can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, TANGO 209 polypeptides, nucleic acids, or modulators thereof, can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

### TANGO 244

A cDNA encoding TANGO 244 was identified by analyzing the sequences of clones present in a human fetal lung cDNA library.

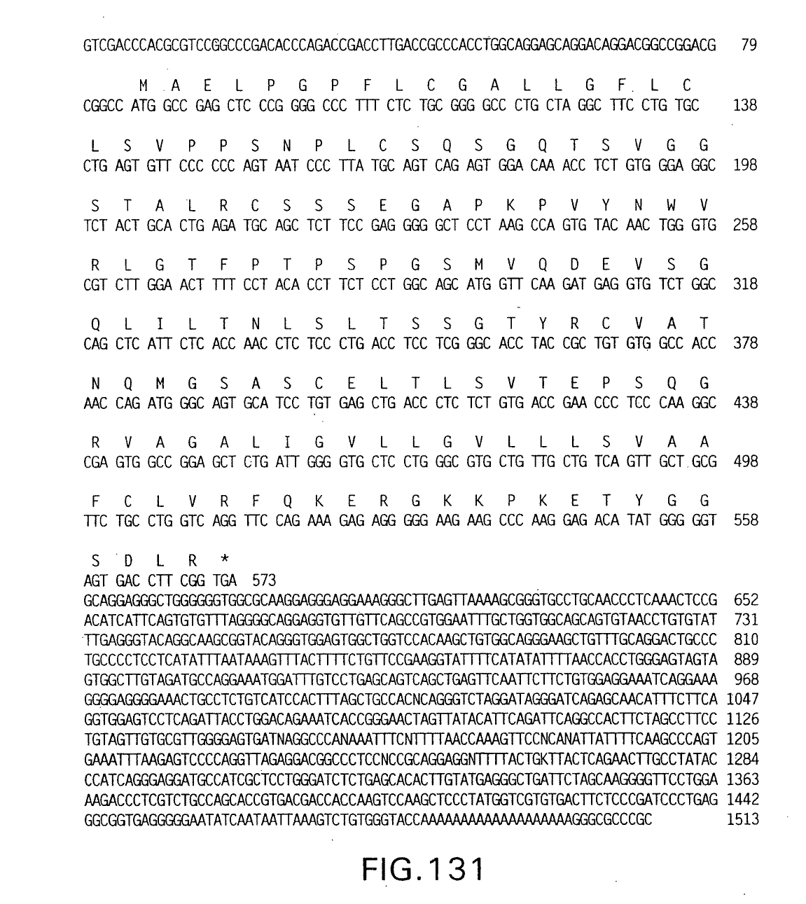

This analysis led to the identification of a clone, Athua62f9, encoding full-length human TANGO 244. The cDNA of this clone is 1513 nucleotides long (FIG. 131; SEQ ID NO:91). The 486 nucleotide open reading frame of this cDNA (nucleotide 85 to nucleotide 570 of SEQ ID NO:91) encodes a 162 amino acid protein (SEQ ID NO:92).

The signal peptide prediction program SIGNALP (Nielsen et al., 1997, Protein Engineering 10:1-6) predicted that human TANGO 244 includes a 26 amino acid signal peptide (amino acid 1 to about amino acid 26 of SEQ ID NO:92) preceding the mature human TANGO 244 protein (corresponding to about amino acid 27 to amino acid 162 of SEQ ID NO:92).

In one embodiment, a TANGO 244 protein contains a signal peptide of about amino acids 1 to 26 (1 to 24, 1 to 25, 1 to 27, or 1 to 28) of SEQ ID NO:92.

Human TANGO 244 is a transmembrane protein having an extracellular domain which extends from about amino acid 27 to about amino acid 119, a transmembrane domain which extends from about amino acid 120 to about amino acid 142, and a cytoplasmic domain which extends from about amino acid 143 to amino acid 162 of SEQ ID NO:92.

Alternatively, in another embodiment, a human TANGO 244 protein contains an extracellular domain at amino acid residues 143 to 162, transmembrane domains at amino acid residues 120 to 142, and a cytoplasmic domain at amino acid residues 27 to 119 of SEQ ID NO:92.

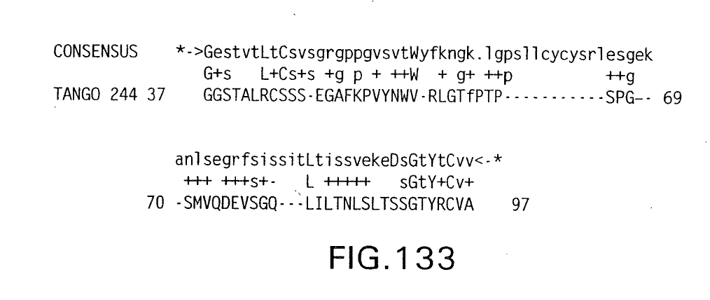

TANGO 244 family members can also include an immunoglobulin domain. Immunoglobulin domains are present in a variety of proteins and are involved in protein-protein and protein-ligand interaction at the cell surface. A consensus hidden Markov model immunoglobulin domain has the sequence. This consensus sequence is shown in FIG. 133 where the more conserved residues in the consensus sequence are indicated by uppercase letters and the less conserved residues in the consensus sequence are indicated by lowercase letters. Human TANGO 244 includes a immunoglobulin domain at amino acids 37 to 97 of SEQ ID NO:92.

In some embodiments of the invention, the domains and the mature protein resulting from the cleavage of such signal peptides are also included herein. For example, the cleavage of a signal peptide consisting of amino acids 1 to 26 results in a mature TANGO 244 protein corresponding to amino acids 27-162 of SEQ ID NO:92. The signal peptide is normally cleaved during possessing of the mature protein.

Human TANGO 244 that has not been post-translationally modified is predicted to have a molecular weight of 16.8 kDa prior to cleavage of its signal peptide and a molecular weight of 14.2 kDa subsequent to cleavage of its signal peptide.

Human TANGO 244 includes an immunoglobulin domain at amino acids 37 to 97 of SEQ ID NO:92. FIG. 133 depicts an alignment of the immunoglobulin domain of human TANGO 244 with a consensus hidden Markov model immunoglobulin domain.

Within human TANGO 244, an N-glycosylation site is present at amino acids 84 to 87. A protein kinase C phosphorylation sites is present at amino acids 92 to 94. N-myristylation sites are present at amino acids 11 to 16, 37 to 42, 91 to 96, 102 to 107, and 122 to 127. An amidation site is present at amino acids 148 to 151.

Clone Athua62f9, which encodes human TANGO 244, was deposited as EpT244 with the American Type Culture Collection (ATCC® 10801 University Boulevard, Manassas, Va. 20110-2209) on Apr. 21, 1999 and assigned Accession Number 207223. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

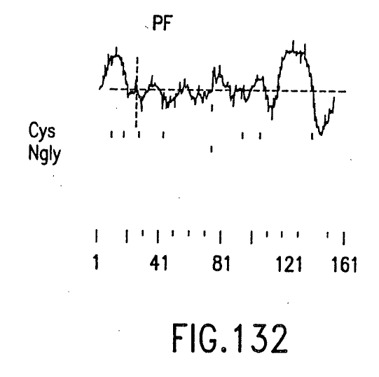

FIG. 132 depicts a hydropathy plot of human TANGO 244. The hydropathy plot indicates that human TANGO 244 has a signal peptide at its amino terminus and an internal hydrophobic region, suggesting that TANGO 244 is a transmembrane protein.

Northern blot analysis of human TANGO 244 expression revealed that human TANGO 244 is expressed in the colon, kidney, liver, and lung.

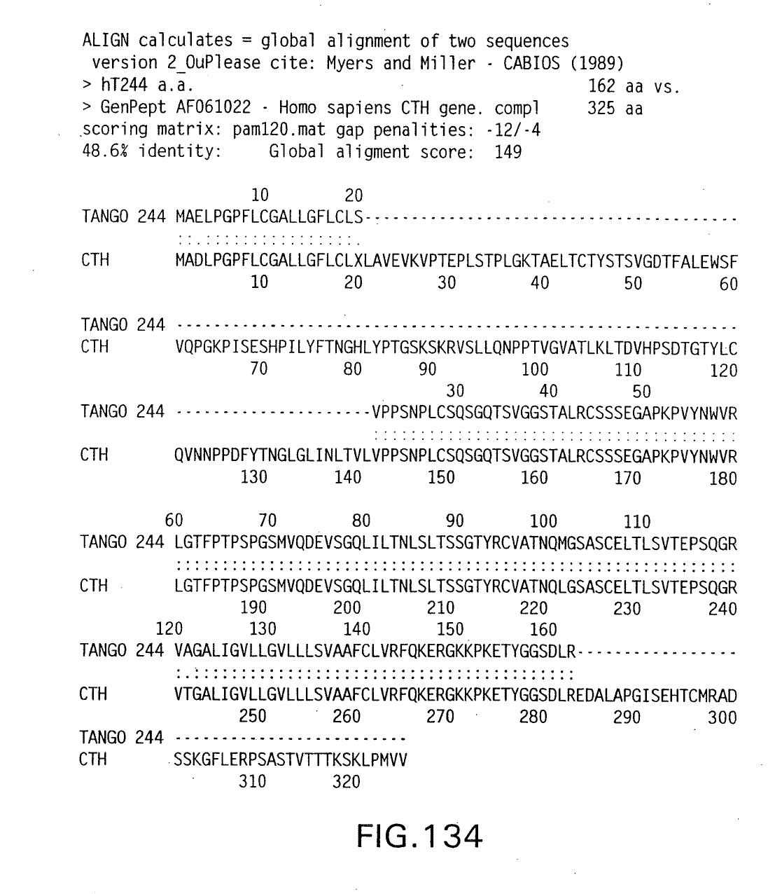

Human TANGO 244 has sequence homology to human CTH (Marcuz et al., 1998, Eur. J. Immunol. 28:4094-4104; Genbank Accession Number AF061022). FIG. 134 depicts an alignment of the amino acid sequence of human TANGO 244 and the amino acid sequence of human CTH. In this alignment, the sequences are 48.6% identical overall. However, there is a substantial region of complete identity. TANGO 244 may act as a immunoglobulin superfamily-type receptor.

### Use of TANGO 244 Nucleic Acids, Polypeptides, and Modulators Thereof

TANGO 244 polypeptides, nucleic acids, and modulators thereof can be used to modulate the function, morphology, proliferation and/or differentiation of cells in the tissues in which they are expressed. Such molecules can be used to treat disorders associated with abnormal or aberrant metabolism or function of cells in the tissues in which they are expressed. Tissues in which TANGO 244 is expressed include, for example, the colon, kidney, liver, and lung. Such disorders include but are limited to lymphoma, leukemia, amyloidosis, scleroderma, mastocytosis.

In one example, TANGO 244 polypeptides, nucleic acids, or modulators thereof can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., idiopathic ulcerative colitis, pseudomembranous colitis, and lymphopathia venereum), tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas) and Crohn's Disease.

In another example, TANGO 244 polypeptides, nucleic acids, or modulators thereof can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 244 polypeptides, nucleic acids, or modulators thereof can be used to treat hepatic (liver) disorders, such as jaundice, hepatic failure, hereditary hyperbiliruinernias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoma, hepatoblastoma, liver cysts and angiosarcoma).

In another example, TANGO 244 polypeptides, nucleic acids, or modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis Goodpasture's syndrome, idiopathic pulmonary hemosiderosis, idiopathic pulmonary fibrosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

Because TANGO 244 includes immunoglobulin domains and has homology to human CTH, TANGO 244 polypeptides, nucleic acids, and modulators thereof can be used to treat disorders involving an immune, allergic or autoimmune response (e.g., arthritis, multiple sclerosis, meningitis, encephalitis, atherosclerosis, or infection).

Further, in light of TANGO 244's pattern of expression in humans, TANGO 244 expression can be utilized as a marker for specific tissues (e.g., tissues of the colon, kidney, liver, or lung) and/or cells (e.g., colon, renal, hepatic, or pulmonary) in which TANGO 244 is expressed. TANGO 244 nucleic acids can also be utilized for chromosomal mapping.

### TANGO 246

A cDNA encoding human TANGO 246 was identified by analyzing the sequences of clones present in a human fetal spleen cDNA library.

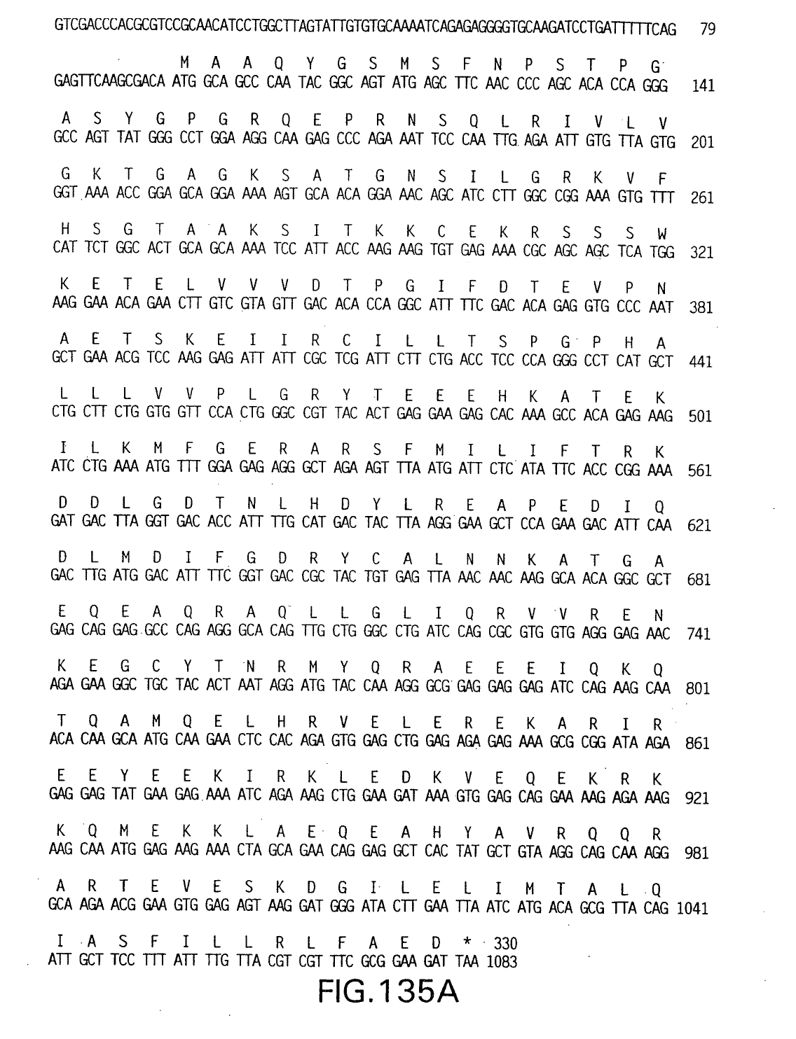

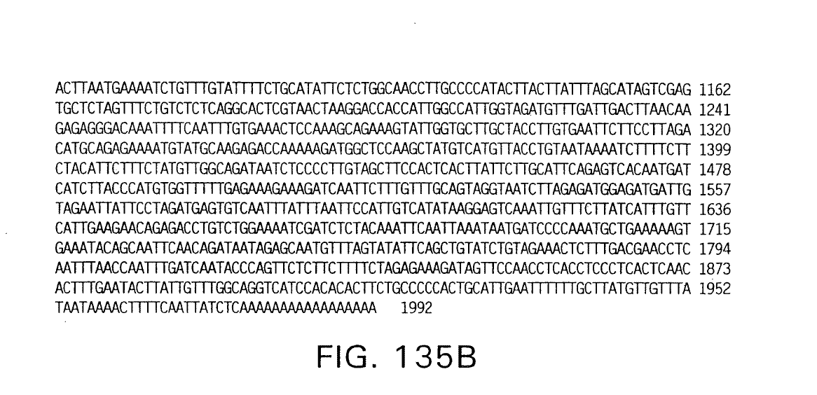

This analysis led to the identification of a clone, Athsa34d2, encoding full-length human TANGO 246. The cDNA of this clone is 1992 nucleotides long (FIG. 135A-135B; SEQ ID NO:93). The 987 nucleotide open reading frame of this cDNA (nucleotide 94 to nucleotide 1080 of SEQ ID NO:93) encodes a 329 amino acid protein (SEQ ID NO:94).

Human TANGO 246 has a hydrophobic domain which extends from about amino acid 306 to about amino acid 323. This could represent a transmembrane domain or an internal signal peptide. This domain follows a domain which extends from about amino acid 1 to about amino acid 305 and is followed by a domain which extends from about amino acid 324 to amino acid 329 of SEQ ID NO:94.

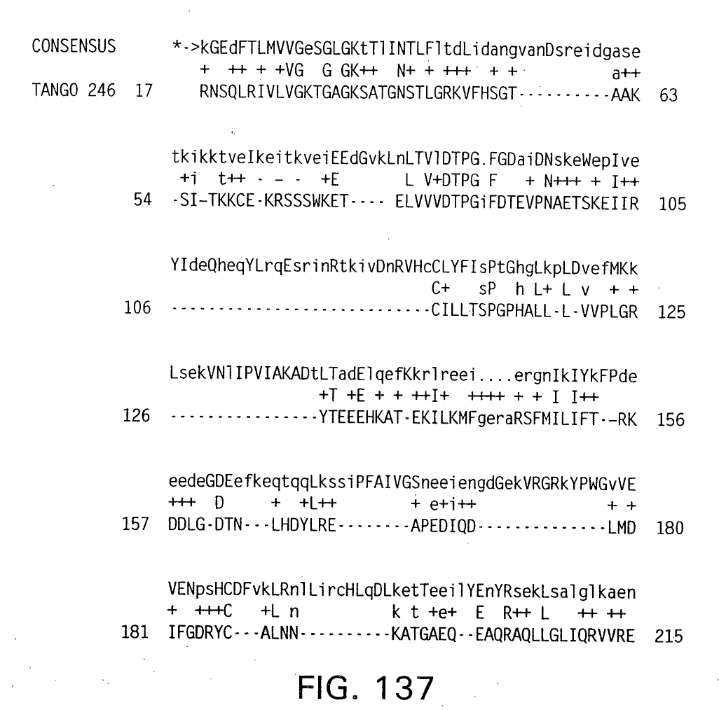

TANGO 246 family members can also include a cell cycle protein domain. A consensus hidden Markov model cell cycle protein domain has the sequence. This consensus sequence is shown in FIG. 137 where the more conserved residues in the consensus sequence are indicated by uppercase letters and the less conserved residues in the consensus sequence are indicated by lowercase letters. Human TANGO 246 includes a cell cycle protein domain at amino acids 27 to 215 of SEQ ID NO:94. Among the proteins which have a cell cycle protein domain are CDC3, CDC10, and CDC11, all of which are important for regulation of the cell cycle. Many proteins which include this domain are GTP binding proteins.

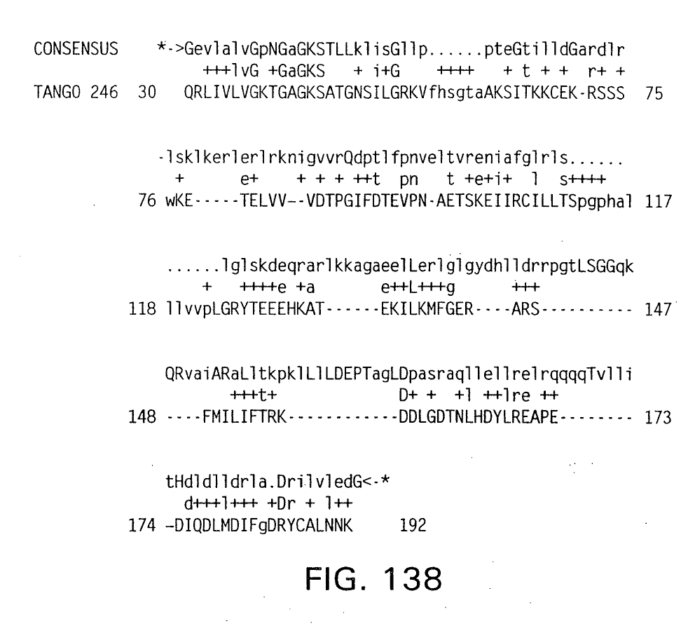

In addition, TANGO 246 family members can also include an ABC transporter domain. A consensus hidden Markov model ABC transporter protein domain has the sequence. This consensus sequence is shown in FIG. 138 where the more conserved residues in the consensus sequence are indicated by uppercase letters and the less conserved residues in the consensus sequence are indicated by lowercase letters. The ABC transporter protein domain of TANGO 246 is located at amino acids 30 to 192 of SEQ ID NO:94. A number of proteins having an ABC transporter protein domain act as active transporters of small hydrophilic molecules (e.g., ions) across cell membranes, including intracellular membranes. In eukaryotes, ABC transporter protein domains are present in multidrug resistance proteins. These protein are involved in extrusion of drugs from cells and play a key role in drug resistance. This domain is also present in cystic fibrosis transmembrane conductance regulator (CFTR), a protein that likely acts as a chloride ion transporter. Many proteins having an ABC transporter domain are ATP binding proteins.

Human TANGO 246 that has not been post-translationally modified is predicted to have a molecular weight of 37.5 kDa.

Within human TANGO 246, a cAMP and cGMP-dependent protein kinase phosphorylation site is present at amino acids 71 to 74. Protein kinase C phosphorylation sites are present at amino acids 66 to 68, 75 to 77, 99 to 101, 134 to 136, 154 to 156, and 222 to 224. Casein kinase II phosphorylation sites are present at amino acids 75 to 78, 99 to 102, 127 to 130, 154 to 157, 194 to 197, and 299 to 302. A tyrosine kinase phosphorylation site is present at amino acids 214 to 221. N-myristylation sites are present at amino acids 40 to 45, 88 to 93, and 219 to 224. An ATP/GTP-binding site motif A is present at amino acids 37 to 44. An amidation site is present at amino acids 51 to 54.

Clone Athsa34d2, which encodes human TANGO 246, was deposited as EpT246 with the American Type Culture Collection (ATCC® 10801 University Boulevard, Manassas, Va. 20110-2209) on Apr. 21, 1999 and assigned Accession Number 207223. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

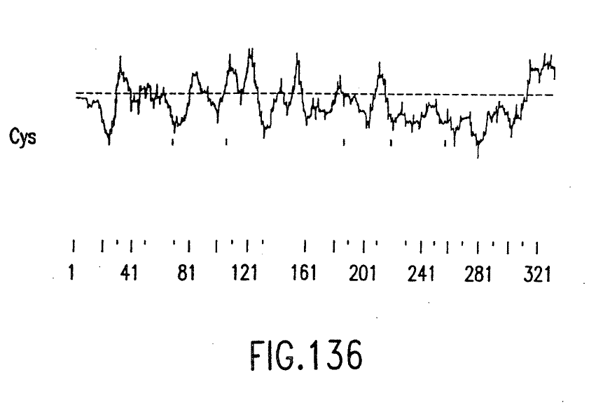

FIG. 136 depicts a hydropathy plot of human TANGO 246. The hydropathy plot indicates the presence of a hydrophobic domain within human TANGO 246, suggesting that human TANGO 246 is either a transmembrane protein or a secreted protein which employs an internal signal peptide.

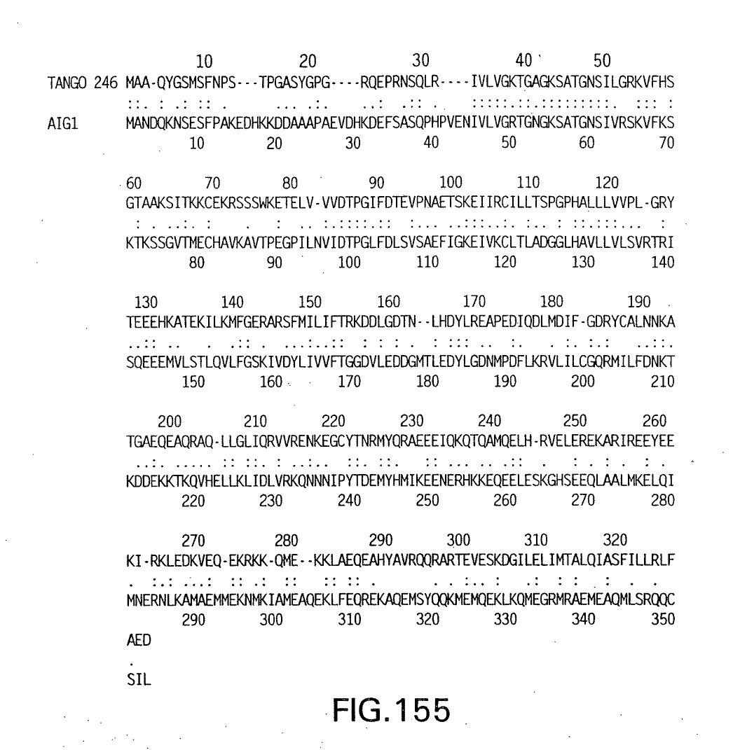

Human TANGO 246 has homology to Arabidopsis thaliana AIG1, a gene which is involved in resistance response (Genbank Accession Number AAC49289: Reuber and Ausubel, 1996, Plant Cell 8:241-249), and Nicotiana tabacum NTGP4 (Genbank Accession Number AAD09518). FIG. 155 depicts an alignment of the amino acid sequence of human TANGO 246 and the amino acid sequence of Arabidopsis thaliana AIG1 (Genbank Accession Number AAC49289. In this alignment, the proteins are 31.2% identical.

### Use of TANGO 246 Nucleic Acids, Polypeptides, and Modulators Thereof

TANGO 246 polypeptides, nucleic acids, and modulators thereof can be used to modulate the function, morphology, proliferation and/or differentiation of cells in the tissues in which they are expressed.

TANGO 246 includes an ABC transporter domain. Proteins having such a domain are involved in disorders of transport of small molecules across cell membranes. Proteins having an ABC transporter domain are known to be involved in cystic fibrosis, hyperinsulinemia, adrenoleukodystrophy, familial intrahepatic cholestasis, sideroblatic anemia and ataxia, Stargardt disease, multidrug resistance, and hyperbilirubinemia II/Dubin-Johnson syndrome. Thus, TANGO 246 polypeptides, nucleic acids, and modulators thereof can be used to treat these and other disorders.

TANGO 246 includes a cell cycle protein domain. Proteins having such a domain are involved in regulation of the cell cycle. Thus, TANGO 246 polypeptides, nucleic acids, and modulators thereof can be used to treat disorders such as Alzheimer's disease, vascular restinosis, polycystic kidney disease, transplant rejection, chronic liver disease, and cancer.

Further, in light of TANGO 246's presence in a human fetal spleen cDNA library, TANGO 246 expression can be utilized as a marker for specific tissues (e.g., lymphoid tissues such as the thymus and spleen) and/or cells (e.g., lymphocytes and splenic) in which TANGO 246 is expressed. TANGO 246 nucleic acids can also be utilized for chromosomal mapping.

### TANGO 275

A cDNA encoding human TANGO 275 was identified by analyzing the sequences of clones present in a human pituitary gland cDNA library.

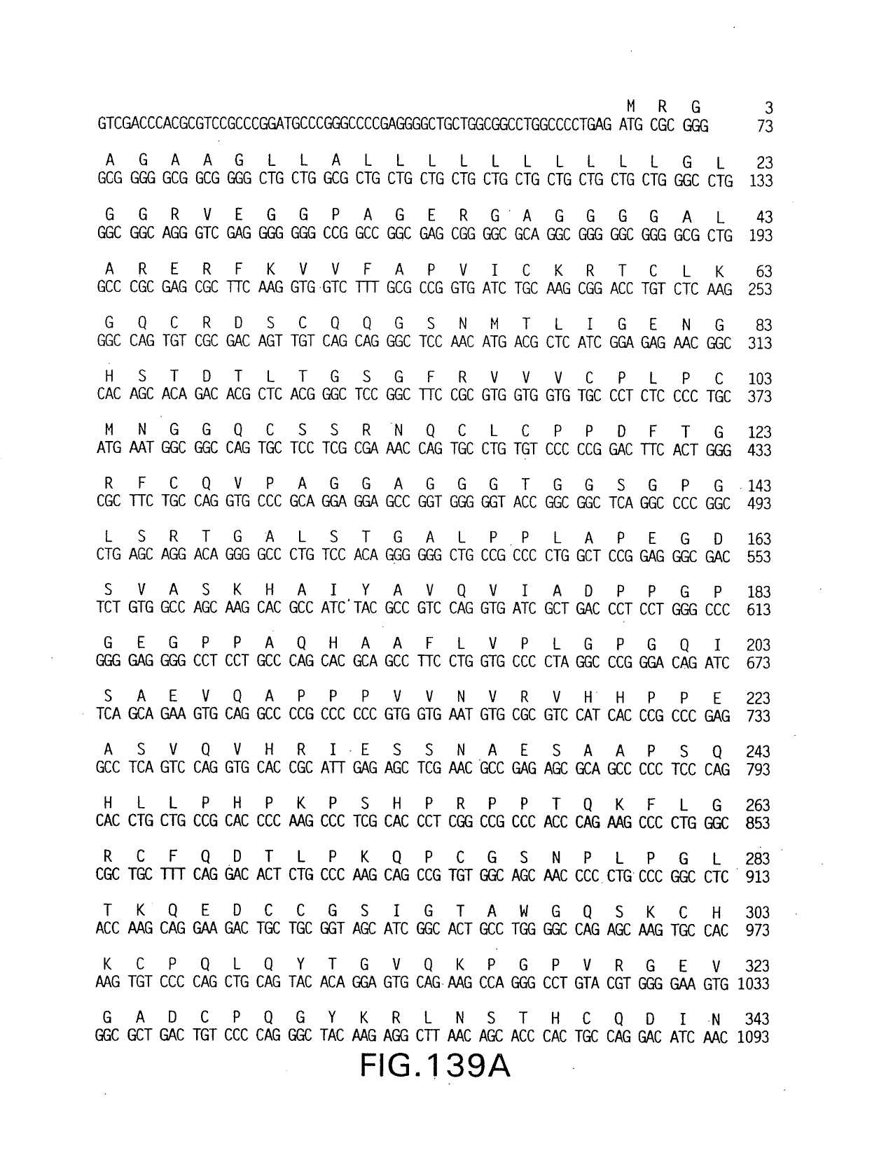

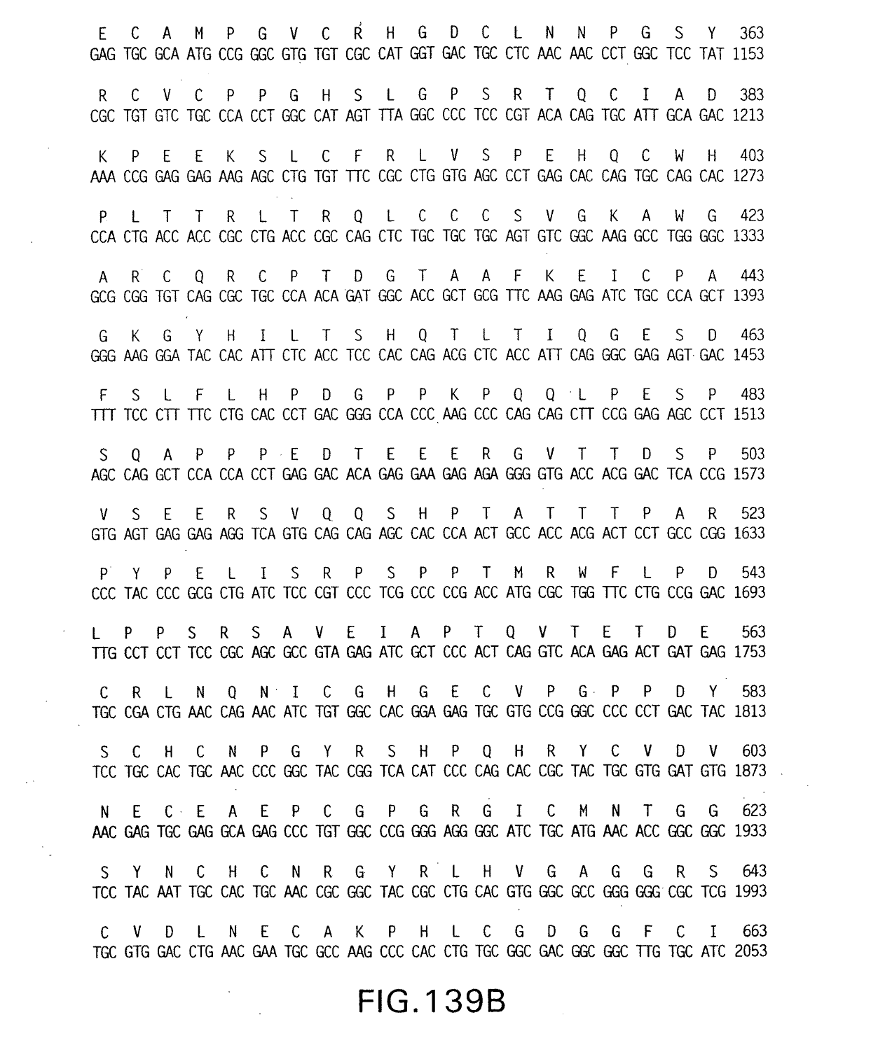

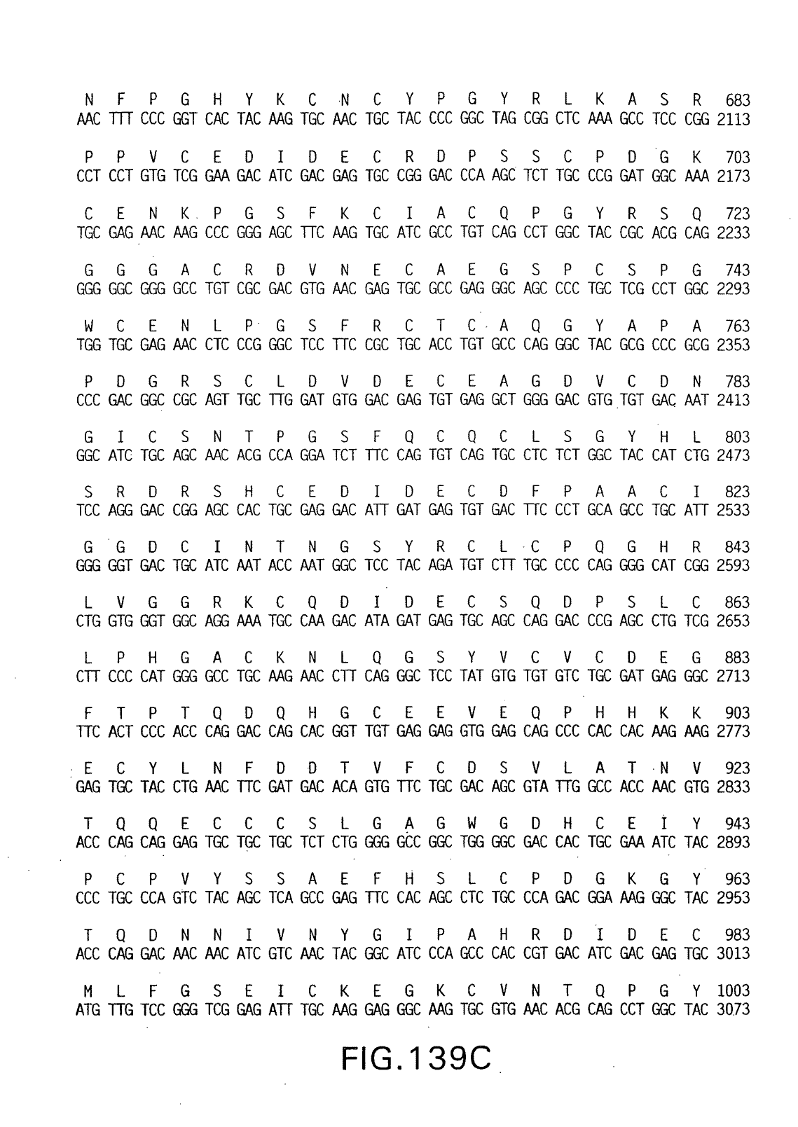

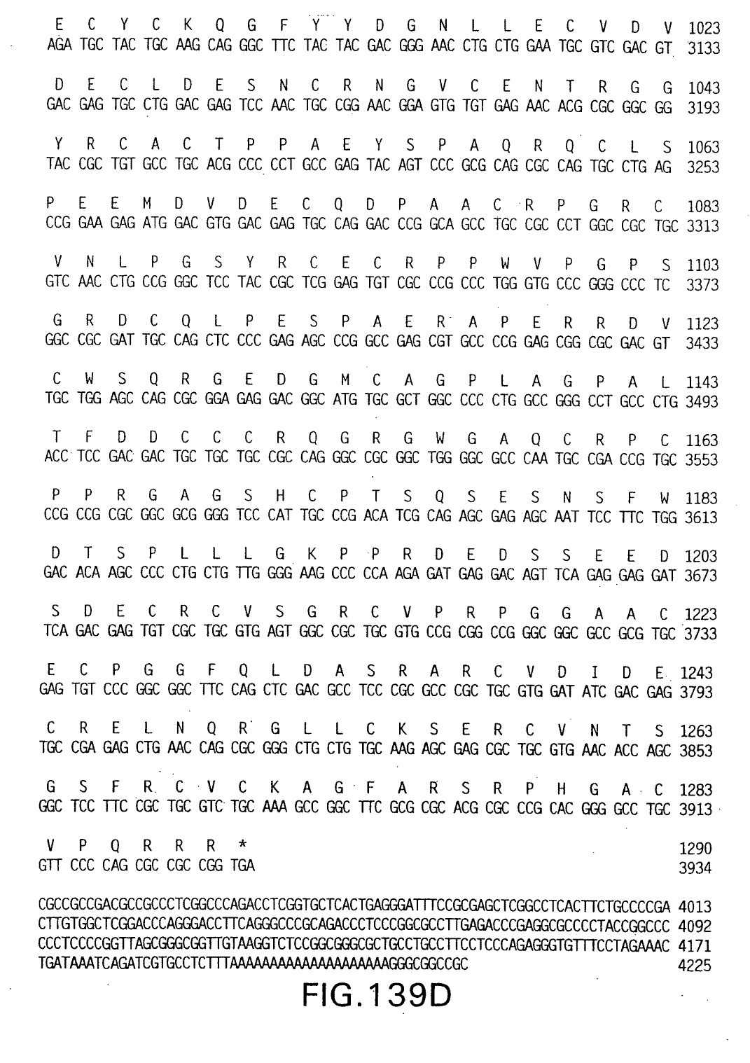

This analysis led to the identification of a clone, Athbb19d1, encoding full-length human TANGO 275. The cDNA of this clone is 4225 nucleotides long (FIG. 139A-139D; SEQ ID NO:95). The 3867 nucleotide open reading frame of this cDNA (nucleotide 565 to nucleotide 3931 of SEQ ID NO:95) encodes a 1289 amino acid protein (SEQ ID NO:96).

The signal peptide prediction program SIGNALP (Nielsen et al., 1997, Protein Engineering 10:1-6) predicted that human TANGO 275 includes a 29 amino acid signal peptide (amino acid 1 to about amino acid 29 of SEQ ID NO:96) preceding the mature human TANGO 275 protein (corresponding to about amino acid 30 to amino acid 1289 of SEQ ID NO:96).

Human TANGO 275 that has not been post-translationally modified is predicted to have a molecular weight of 137.9 kDa prior to cleavage of its signal peptide and a molecular weight of 135.3 kDa subsequent to cleavage of its signal peptide.

In one embodiment, a TANGO 275 protein contains a signal peptide of about amino acids 1 to 29 (1 to 27, 1 to 28, 1 to 30, 1 to 31) of SEQ ID NO:96.

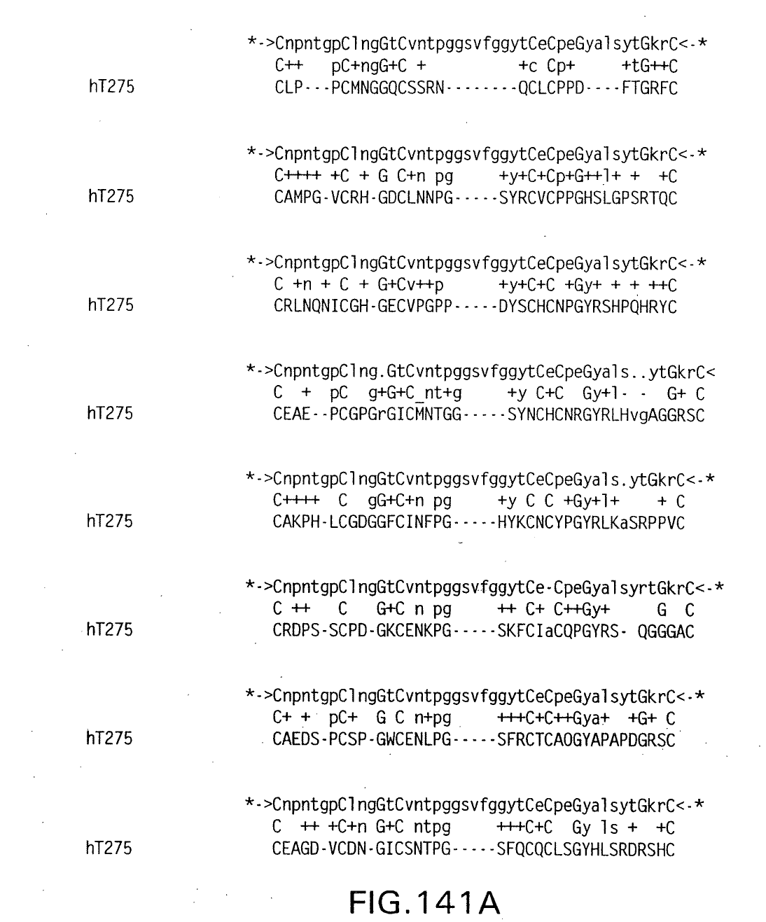

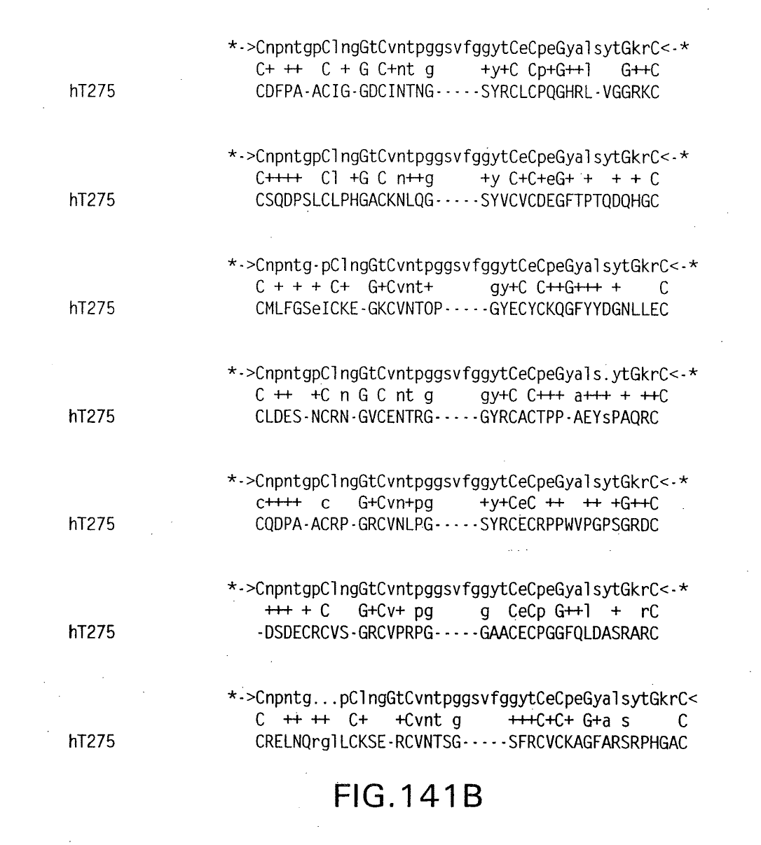

TANGO 275 family members can include an EGF-like domain. A consensus hidden Markov model EGF-like domain has the sequence shown in the alignments depicted in FIG. 141A-141B, where the more conserved residues in the consensus sequence are indicated by uppercase letters and the less conserved residues in the consensus sequence are indicated by lowercase letters. Human TANGO 275 includes EFG-like domains at amino acids 99 to 126, 345 to 380, 564 to 600, 606 to 644, 650 to 687, 693 to 728, 734 to 769, 775 to 810, 816 to 850, 856 to 893, 983 to 1020, 1026 to 1061, 1072 to 1107, 1203 to 1238, and 1244 to 1283 of SEQ ID NO:96. One or more EGF-like domains (e.g., 1, 2, 4, 8, 13, 17, or 44 copies) are found in the extracellular domain of a wide range of proteins of transmembrane and wholly secreted proteins having diverse function. The consensus EGF-like domain sequence includes six cysteines, all of which are thought to be involved in disulfide bonds.

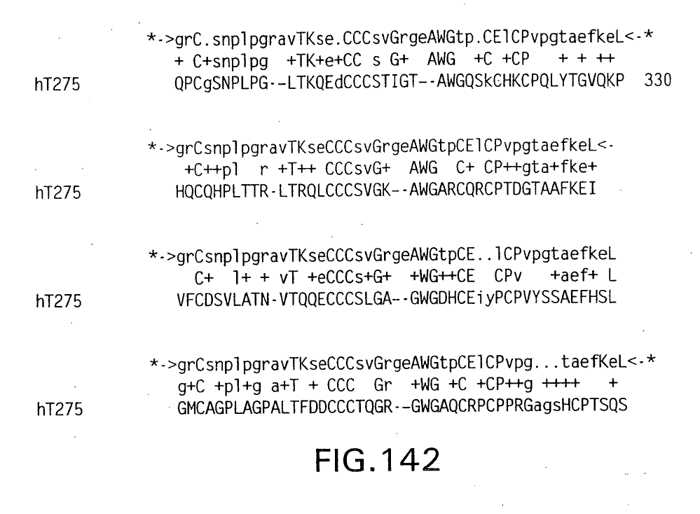

TANGO 275 family members can include a transforming growth factor β binding protein-like domains (TB domains). A consensus hidden Markov model TB domain has the amino acid sequence. This consensus sequence is shown in FIG. 142 where the more conserved residues in the consensus sequence are indicated by uppercase letters and the less conserved residues in the consensus sequence are indicated by lowercase letters. Human TANGO 275 includes TB domains at amino acids 273 to 316, 399 to 440, 913 to 956, and 1132 to 1177 of SEQ ID NO:96. A TB domain is found in matrix fibrils (Yuan et al., 1997, EMBO J. 16:6659-66).

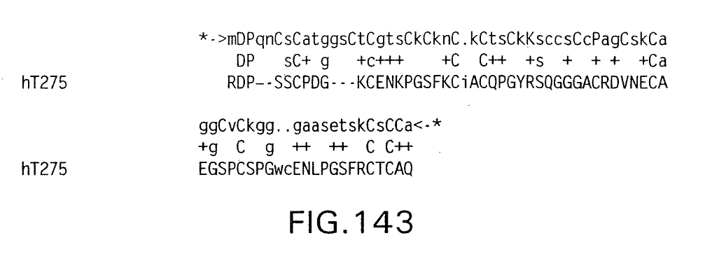

TANGO 275 family members can include a metallothionein domain. A consensus hidden Markov model metallothionein domain has the amino acid sequence. This consensus sequence is shown in FIG. 143 where the more conserved residues in the consensus sequence are indicated by uppercase letters and the less conserved residues in the consensus sequence are indicated by lowercase letters. Human TANGO 275 includes a metallothionein domain at amino acids 694 to 708 of SEQ ID NO:96. Metallothionein domains are found in proteins which bind heavy metals (e.g., copper, zinc, cadmium, and nickel) through thiolate bonds.

Human TANGO 275 includes EFG-like domains at amino acids 99 to 126, 345 to 380, 564 to 600, 606 to 644, 650 to 687, 693 to 728, 734 to 769, 775 to 810, 816 to 850, 856 to 893, 983 to 1020, 1026 to 1061, 1072 to 1107, 1203 to 1238, and 1244 to 1283 of SEQ ID NO:96. An alignment of each of the EGF-like domains of human TANGO 275 with a consensus hidden Markov model EGF-like domain is shown in FIG. 141A-141B.

Human TANGO 275 includes transforming growth factor β binding protein like domains (TB domains) at amino acids 273 to 316, 399 to 440, 913 to 956, and 1132 to 1177 of SEQ ID NO:96. An alignment of each of the TB domains of human TANGO 275 with a consensus hidden Markov model TB domain is shown in FIG. 142.

Human TANGO 275 includes a metallothionein domain at amino acids 694 to 708 of SEQ ID NO:96. An alignment of the metallothionein domain of human TANGO 275 with a consensus hidden Markov model metallothionein domain is shown in FIG. 143.

N-glycosylation sites are present at amino acids 75 to 78, 335 to 338, 831 to 834, 922 to 925, and 1261 to 1264 of SEQ ID NO:96.

Clone Athbb19d1, which encodes human TANGO 275, was deposited as EpT275 with the American Type Culture Collection (ATCC® 10801 University Boulevard, Manassas, Va. 20110-2209) on Apr. 21, 1999 and assigned Accession Number 207220. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

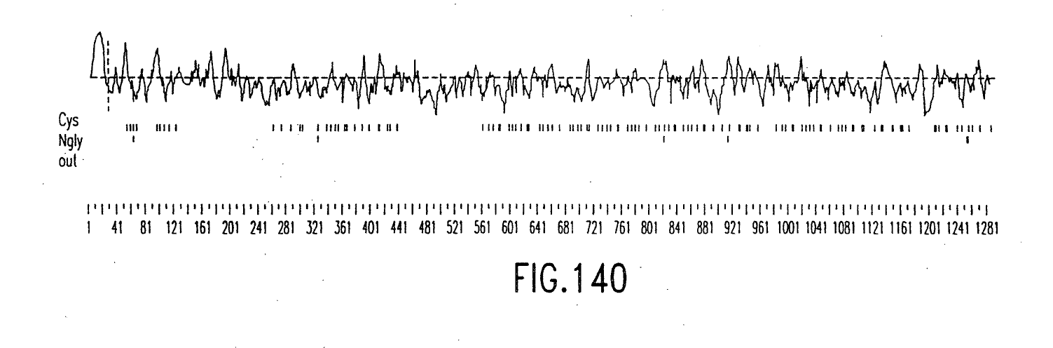

FIG. 140 depicts a hydropathy plot of human TANGO 275. The hydropathy plot indicates that human TANGO 275 has a signal peptide at its amino terminus, suggesting that human TANGO 275 is a secreted protein.

Transcript analysis suggests that there are several splice variants of human TANGO 275.

Human TANGO 275 appears to be the human homolog of a mouse latent transforming growth factor-β binding protein 3 (LTBP-3; Yin et al., J. Biol. Chem. 270:10147-60, 1995; Genbank Accession Number RL40459; PCT Application WO 95/22611; GENSEQ Accession Number R79475).

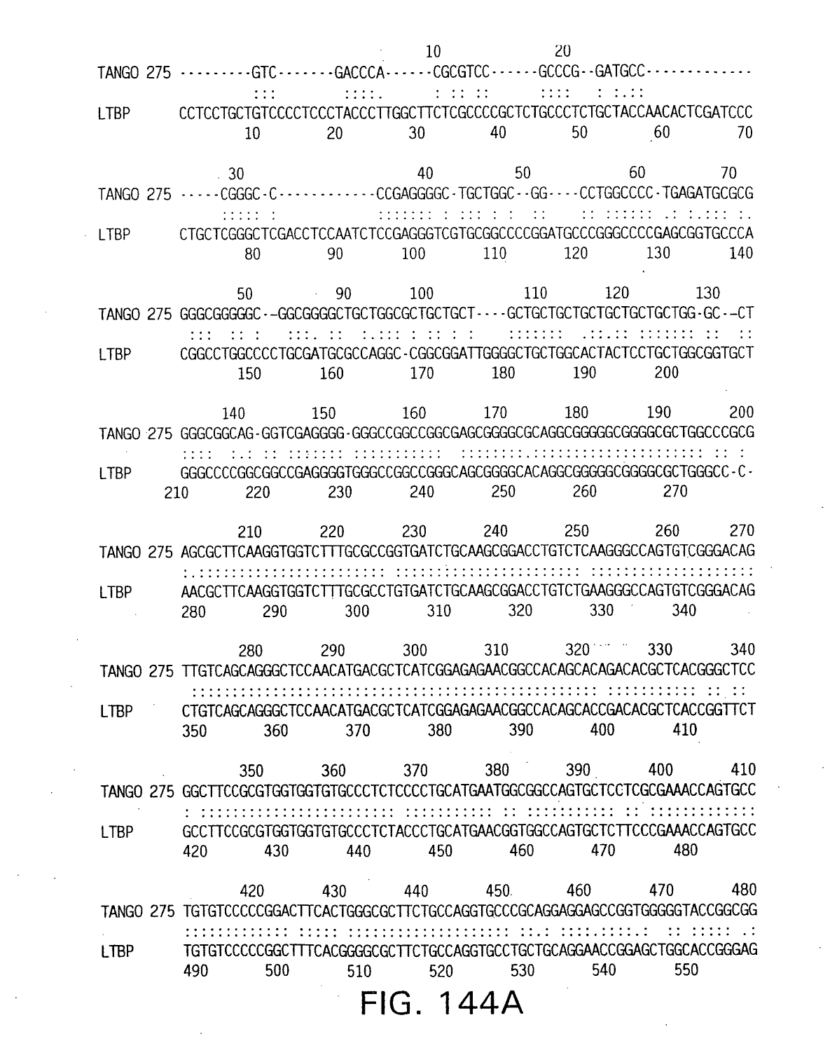

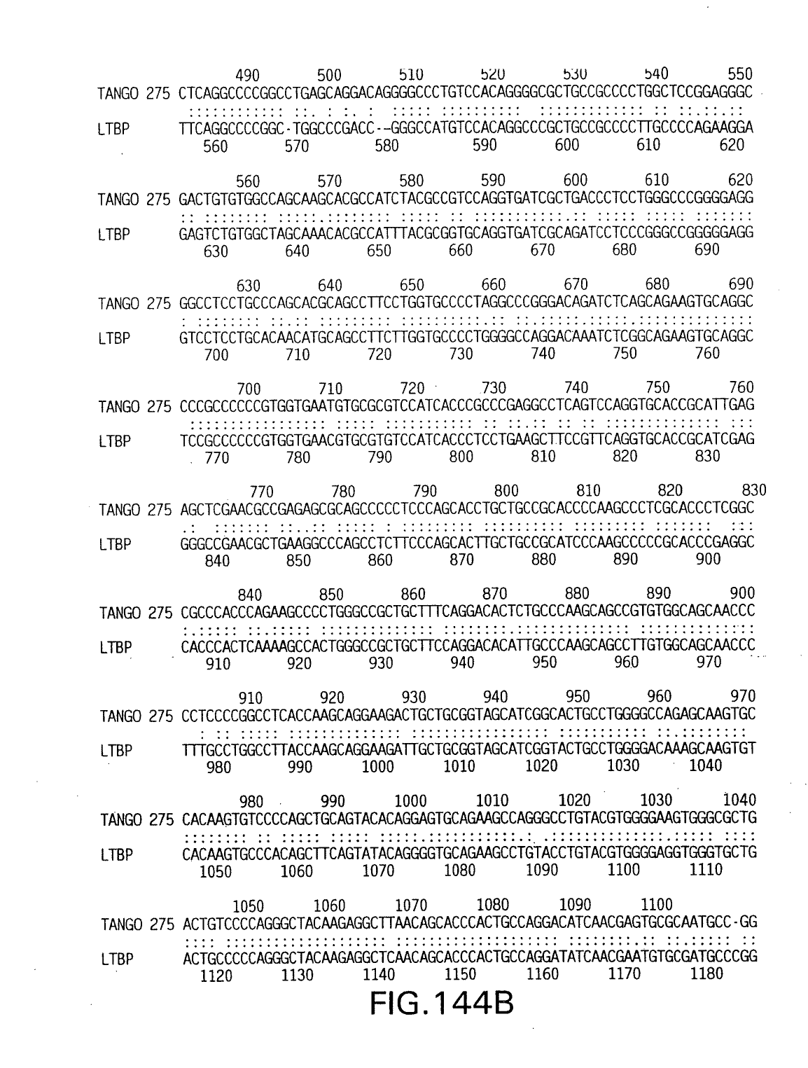

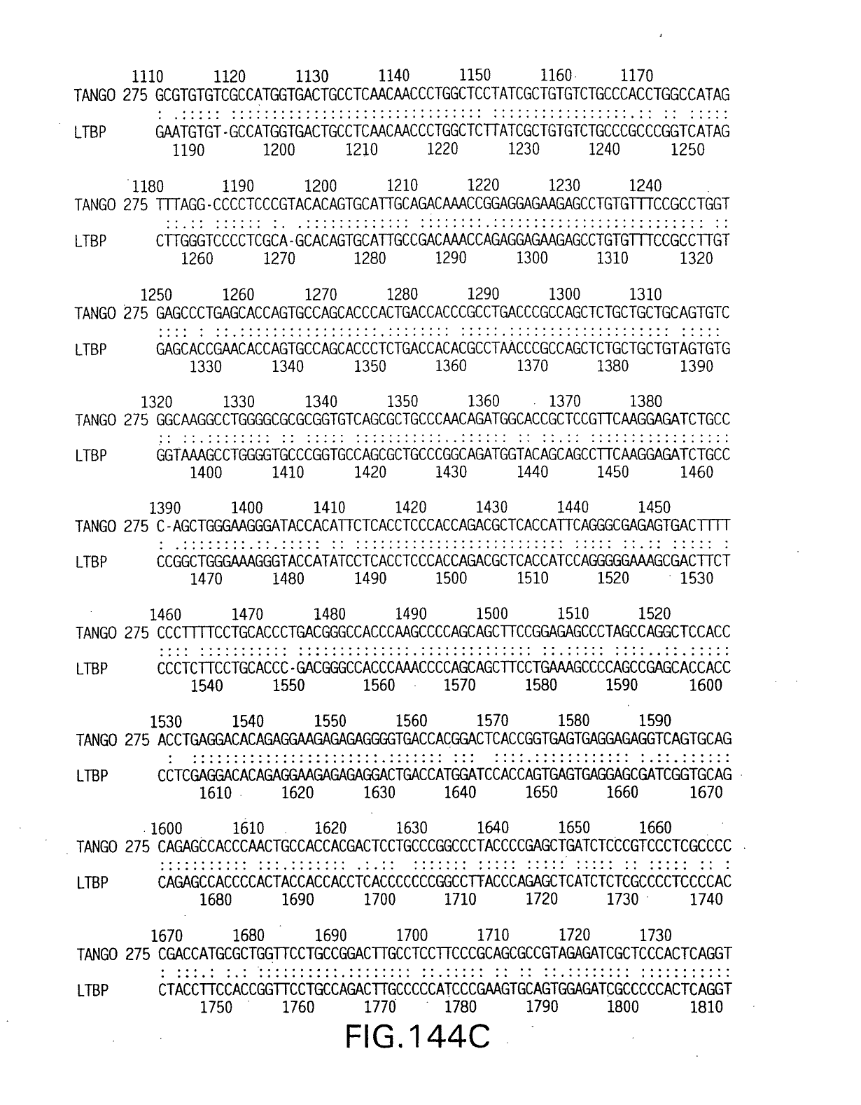

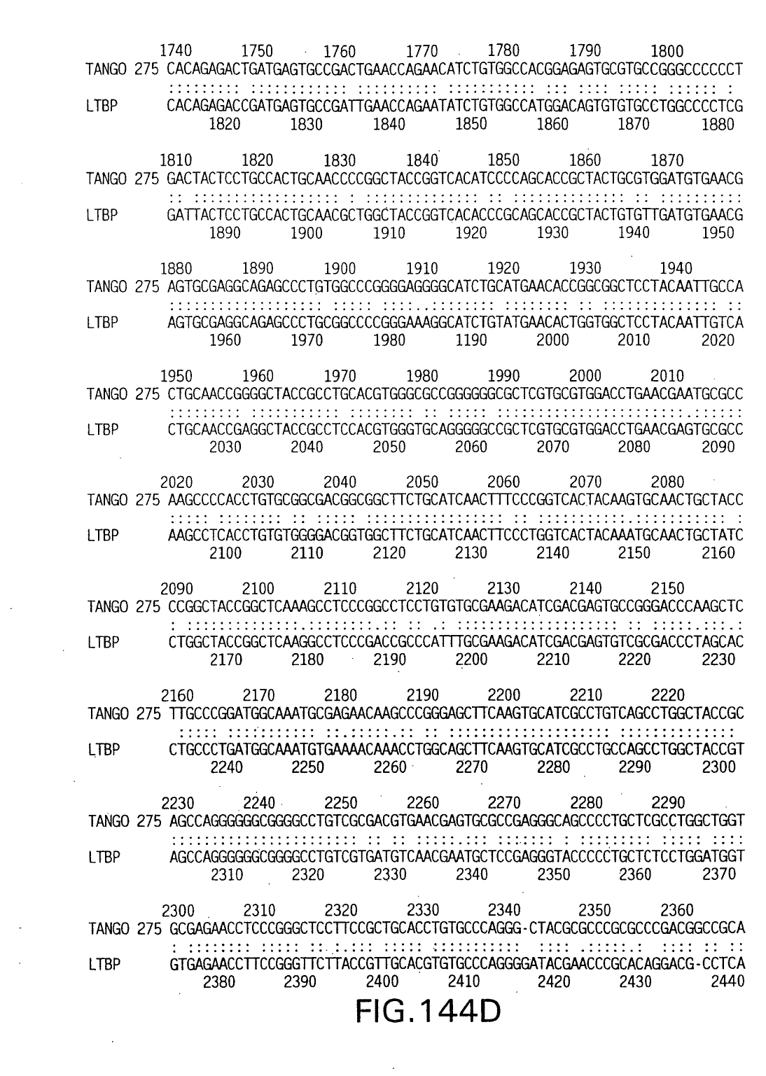

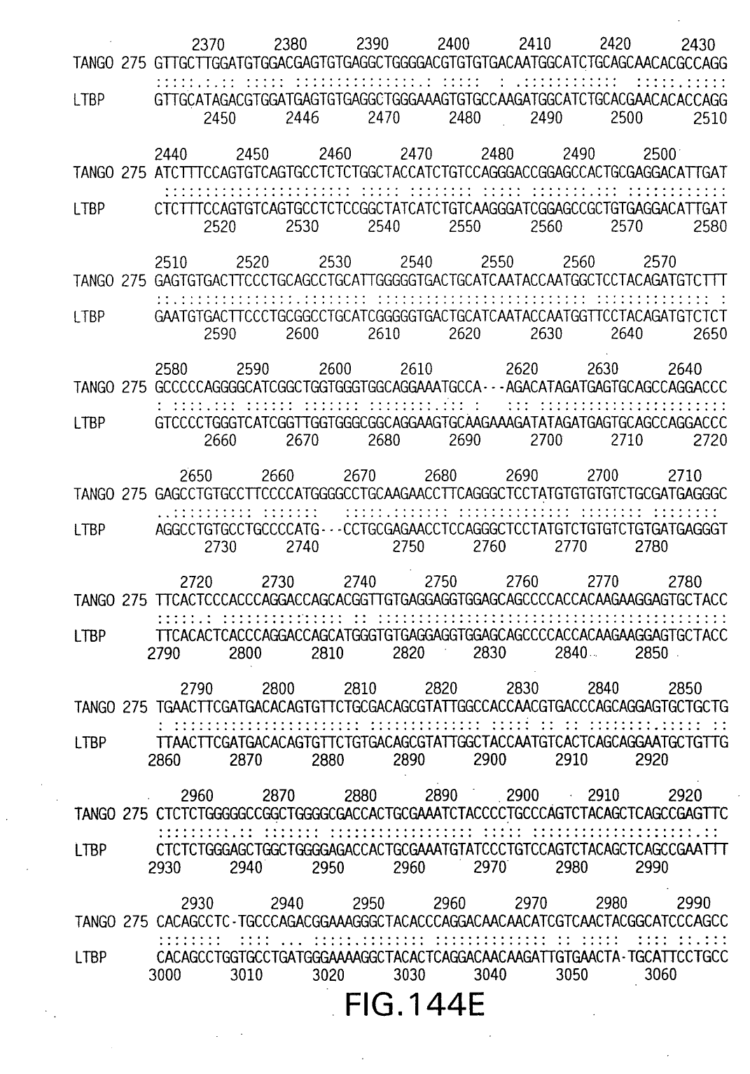

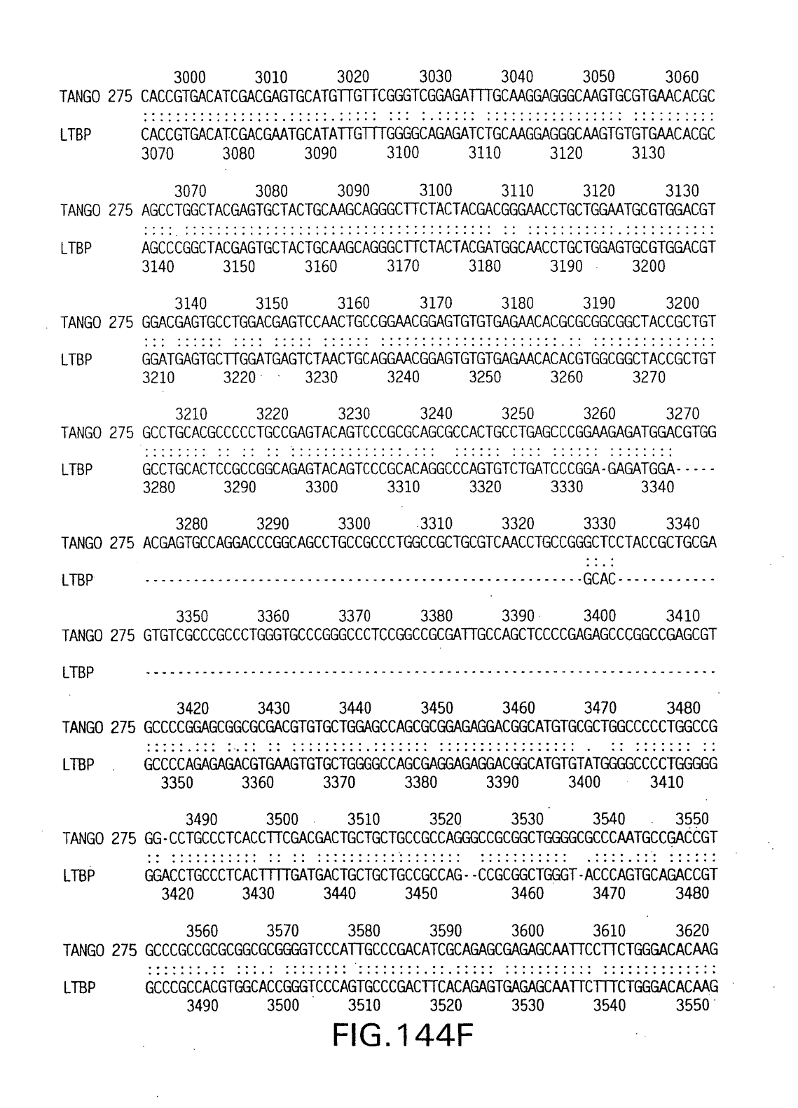

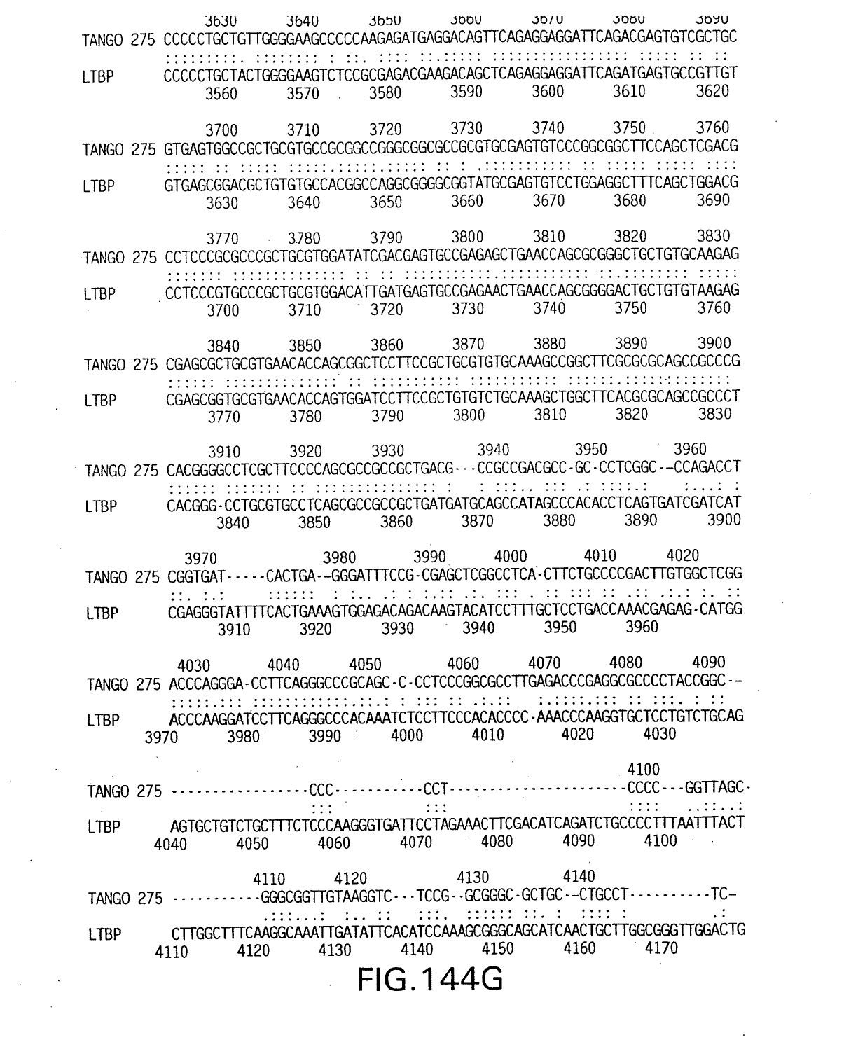

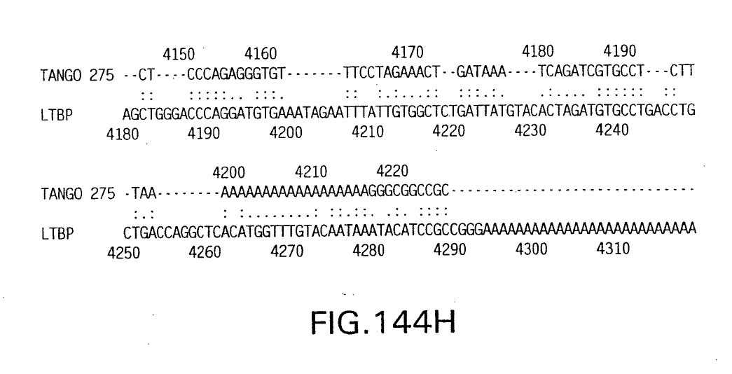

FIG. 144A-144H depicts an alignment of the nucleotide sequence of human TANGO 275 and the nucleotide sequence of mouse LTBP-3 (Genbank Accession Number L40459). This alignment was created using ALIGN (version 2.0; PAM120 scoring matrix; gap length penalty of 12; gap penalty of 4). In this alignment, the sequences are 77.1% identical.

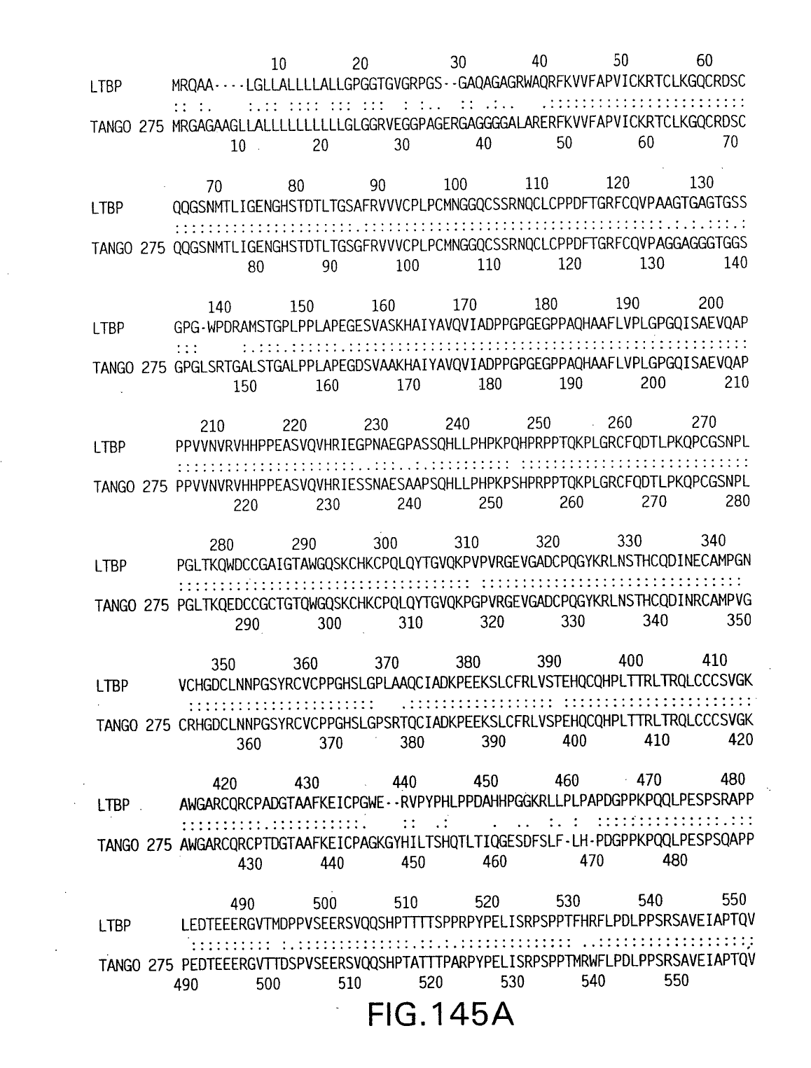

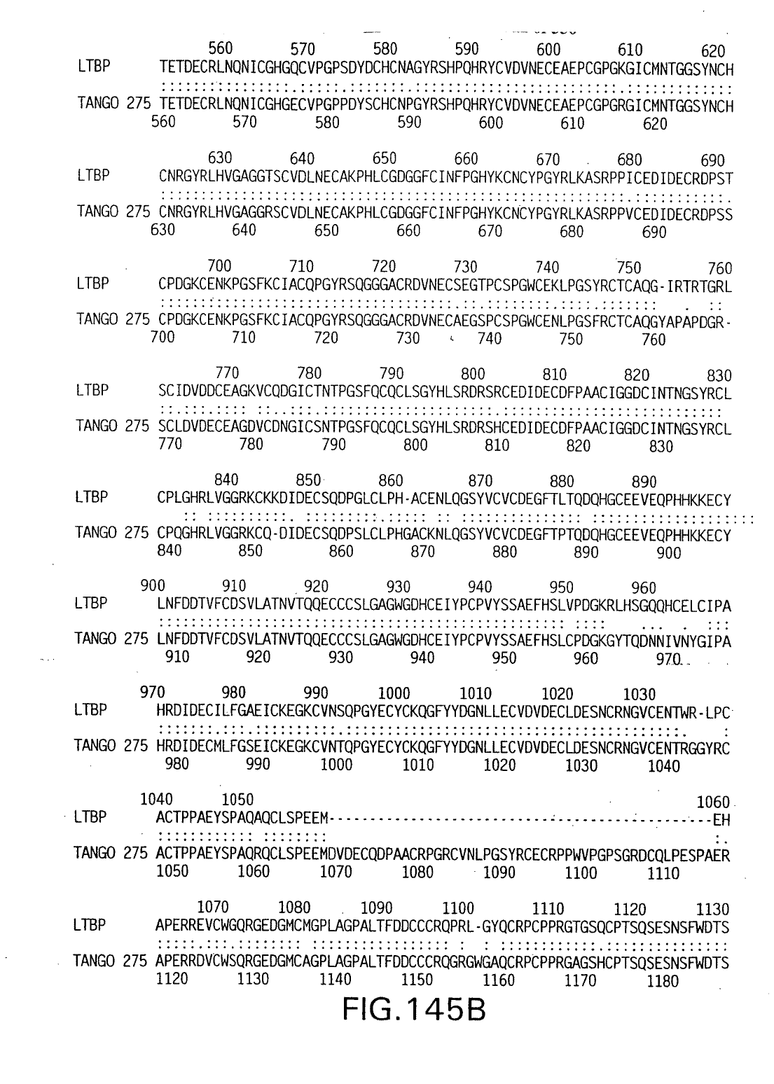

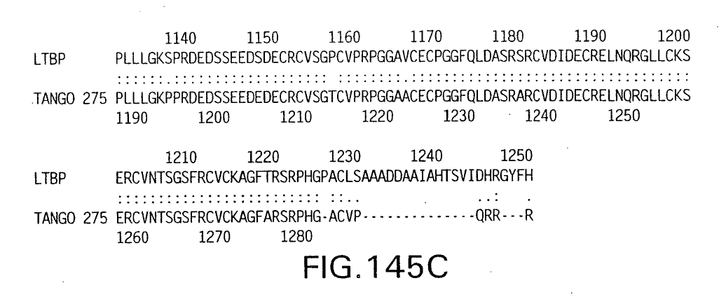

FIG. 145A-145C depicts an alignment of the amino acid sequence of human TANGO 275 and the amino acid sequence of mouse LTBP-3 (GENSEQ R79475). This alignment was created using ALIGN (version 2.0; PAM 120 scoring matrix; gap length penalty of 12; gap penalty of 4). In this alignment, the sequences are 82.8% identical.

Northern blot analysis of human TANGO 275 expression revealed that human TANGO 275 is expressed at a high level in the heart and at a moderate level in the brain, placenta, lung, liver, skeletal muscle, kidney and pancreas.

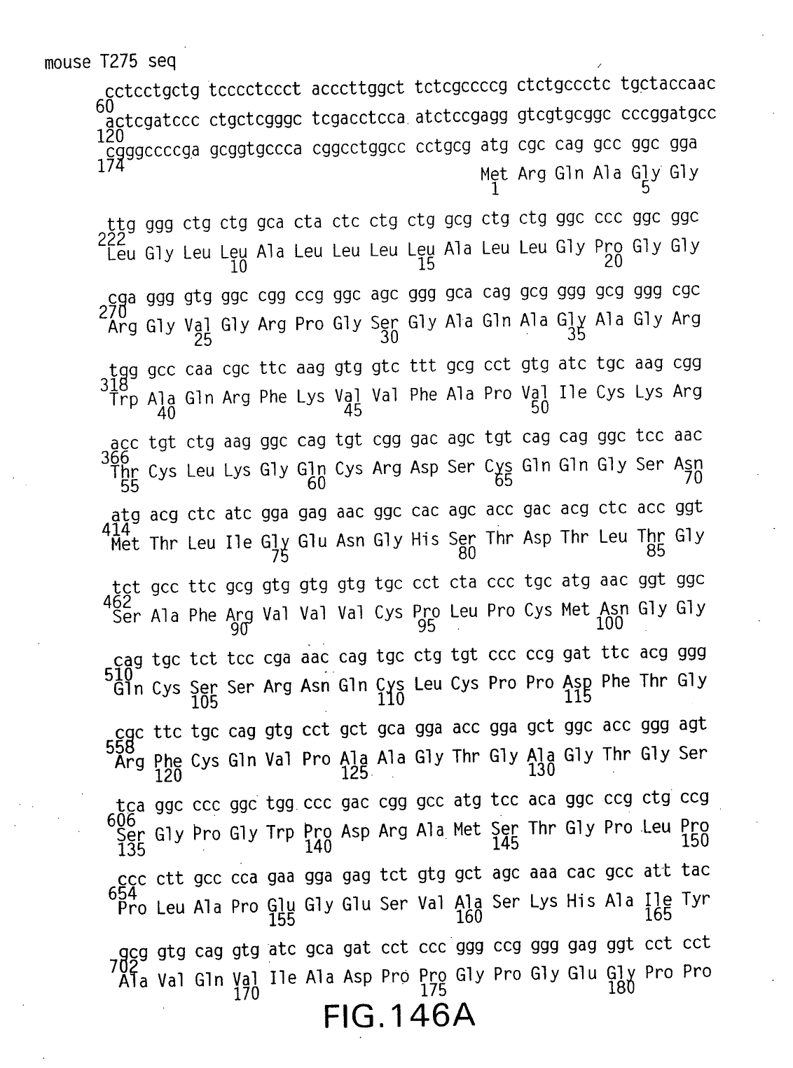

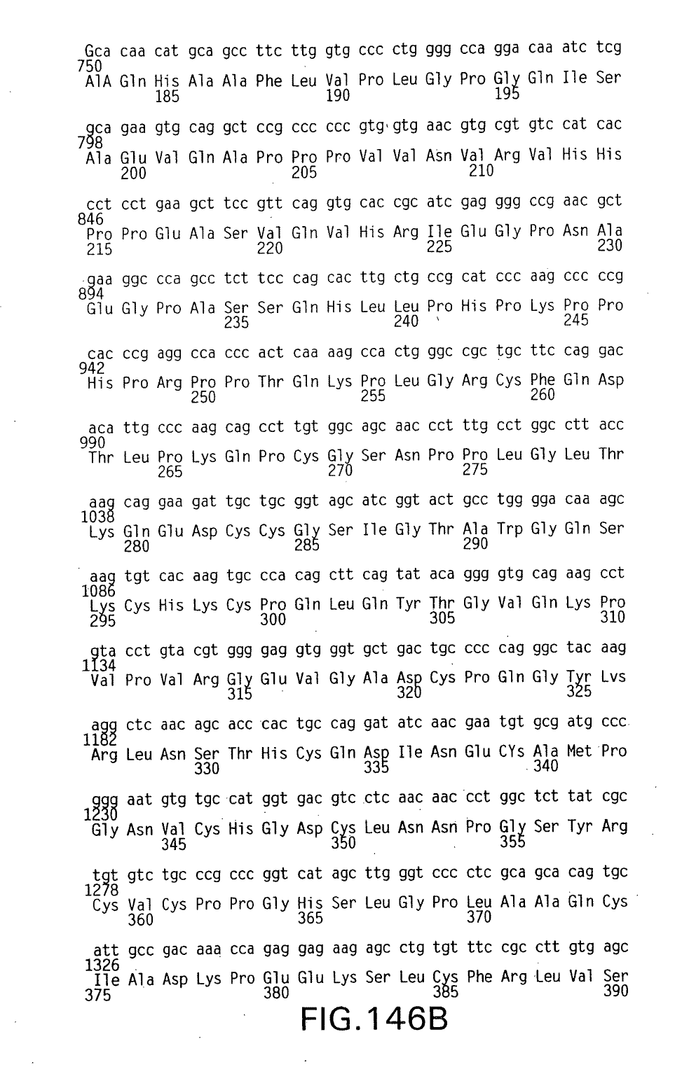

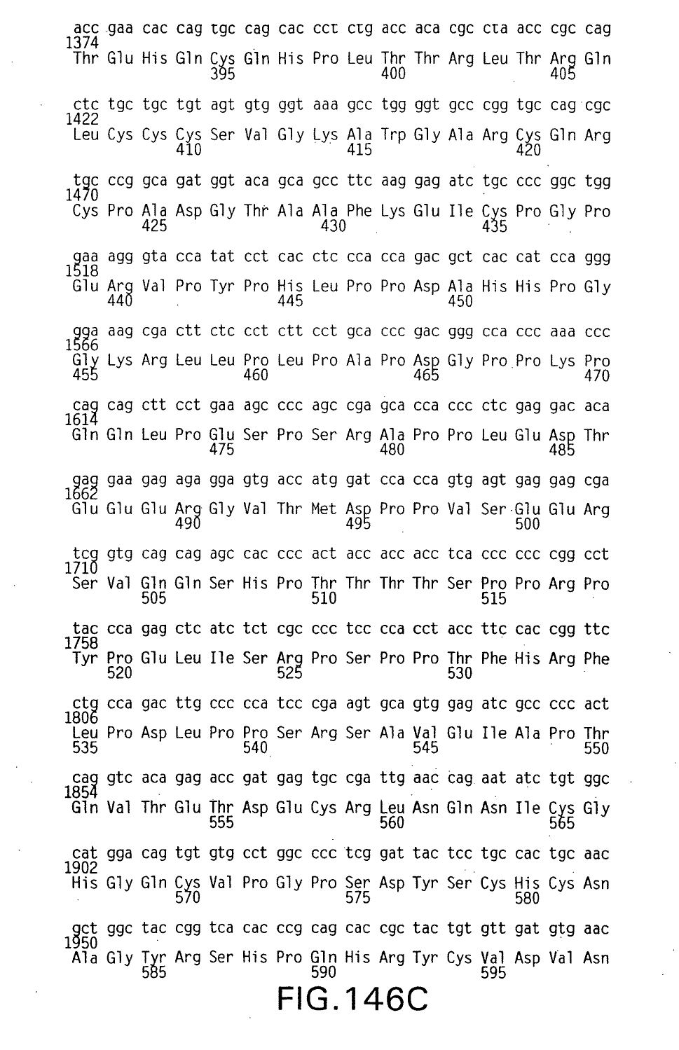

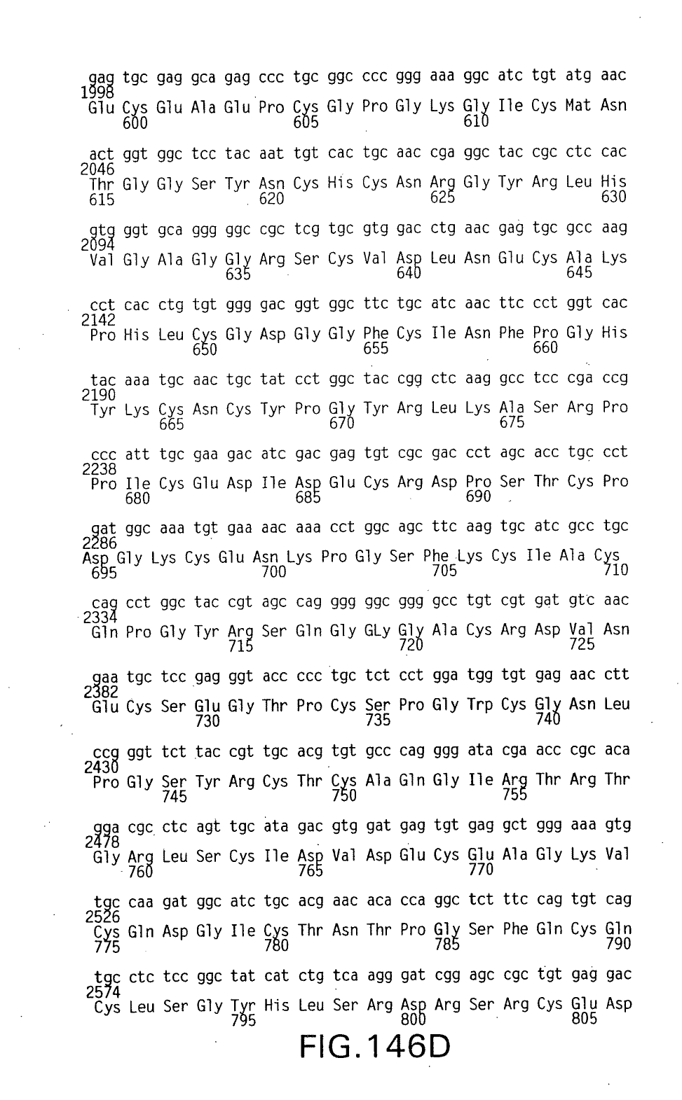

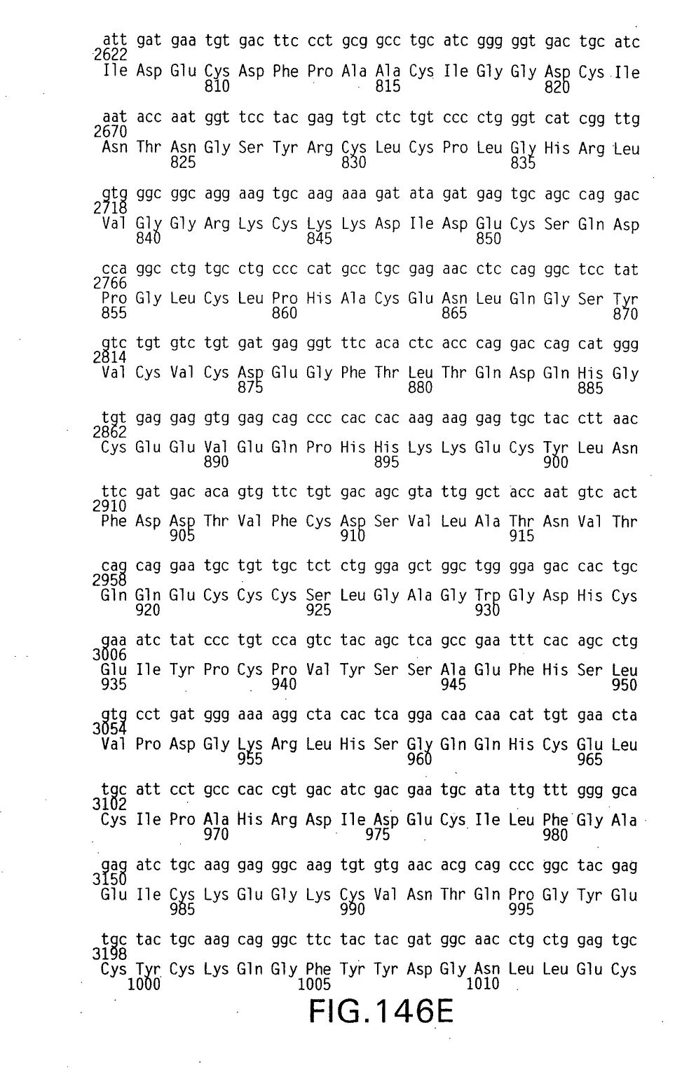

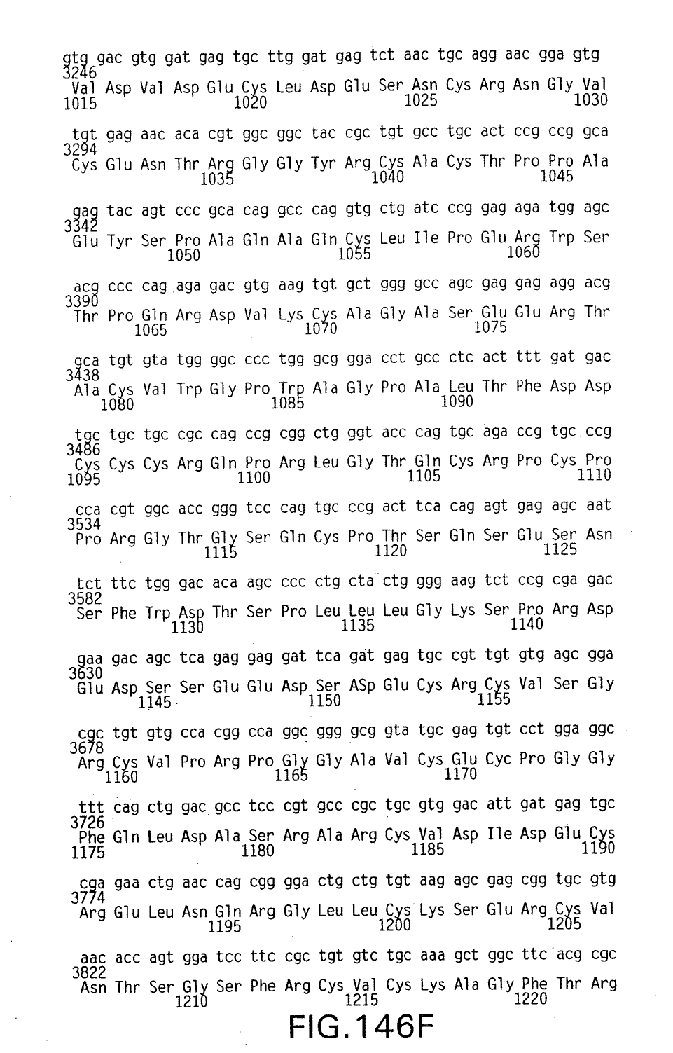

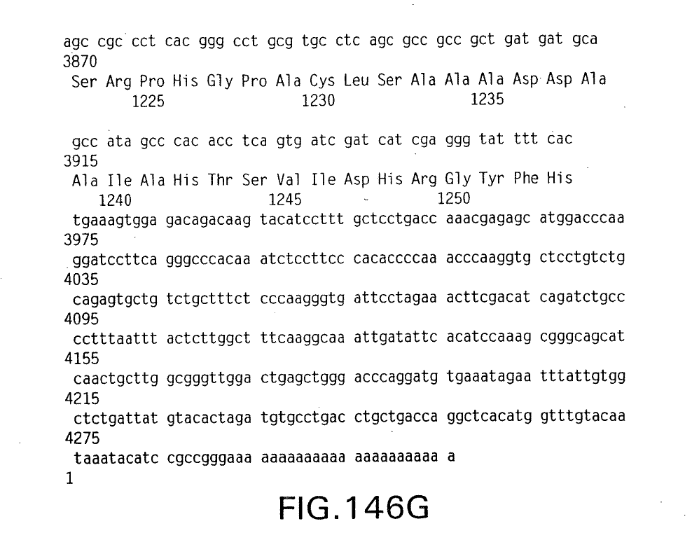

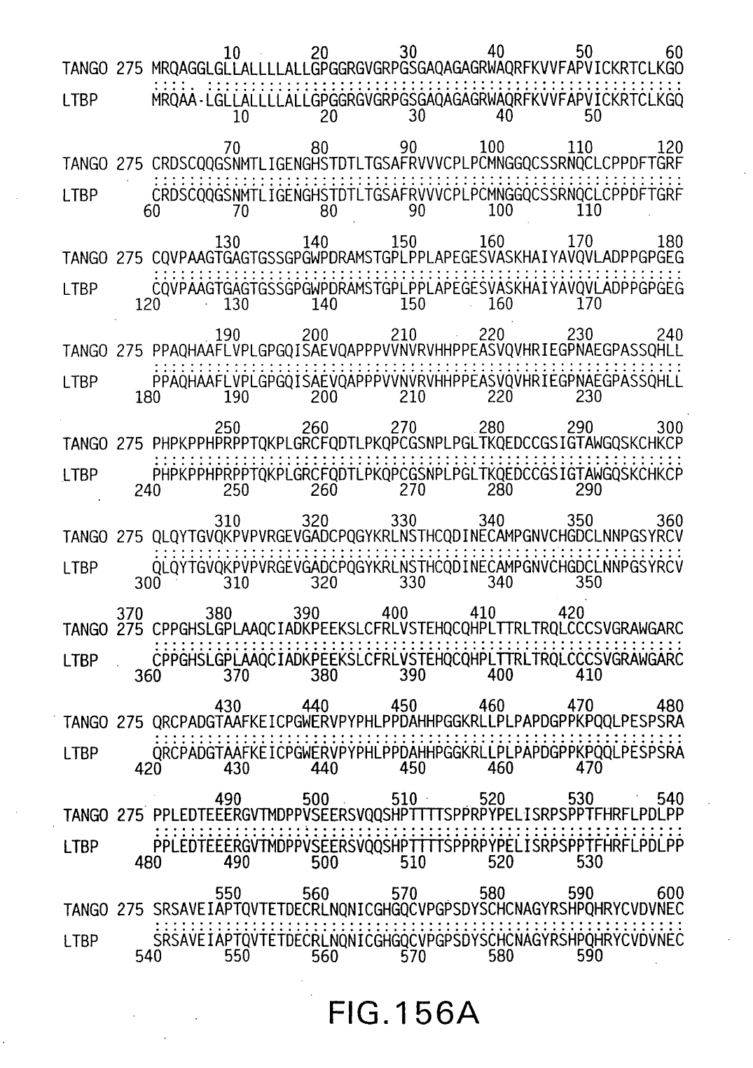

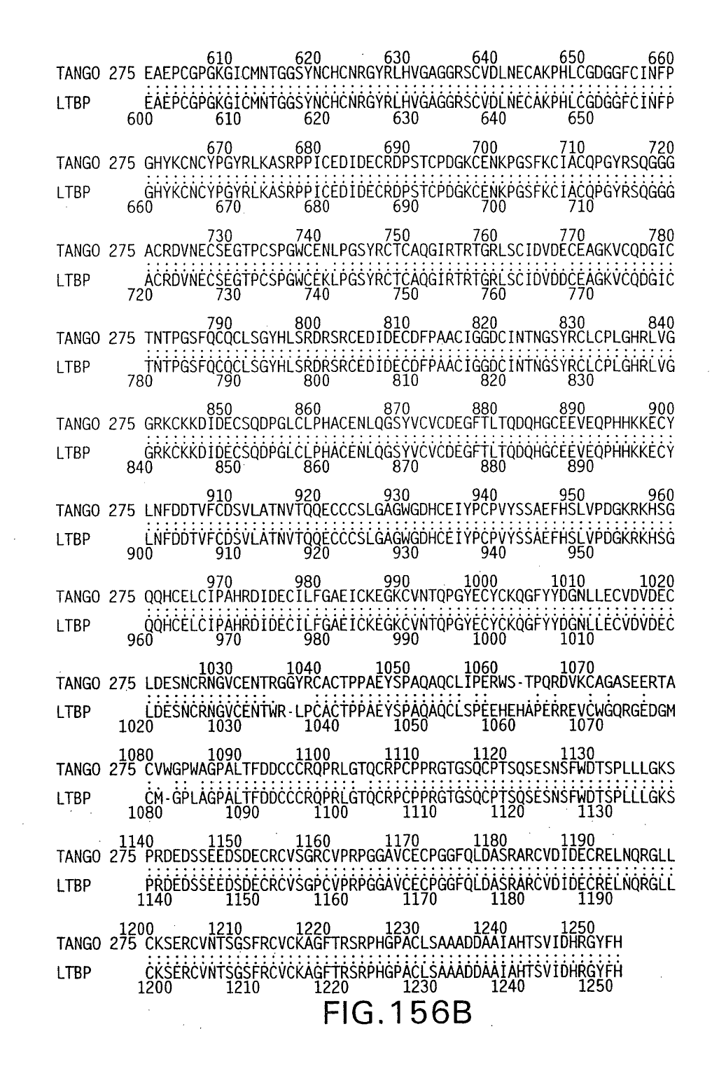

A mouse TANGO 275 cDNA was identified. The cDNA of this clone is 4376 nucleotides long (FIG. 146A-146G; SEQ ID NO:97). The 3759 nucleotide open reading frame of this cDNA, nucleotides, encodes a 1253 amino acid protein (SEQ ID NO:98). FIG. 156A-156B depicts an alignment of the amino acid sequence encoded by this mouse TANGO 275 cDNA clones and the amino acid sequence of mouse LTBP-3 (GENSEQ Accession Number R79475). This alignment was created using ALIGN (version 2.0; PAM 120 scoring matrix, gap length penalty of 12; gap penalty of 4). In this alignment, the sequences are 97.4% identical.

### Use of TANGO 275 Nucleic Acids, Polypeptides, and Modulators Thereof

TANGO 275 polypeptides, nucleic acids, and modulators thereof can be used to modulate the function, morphology, proliferation and/or differentiation of cells in the tissues in which they are expressed. Such molecules can be used to treat disorders associated with abnormal or aberrant metabolism or function of cells in the tissues in which they are expressed. Tissues in which TANGO 275 is expressed include, for example, pancreas, adrenal medulla, adrenal cortex, kidney, thyroid, testis, stomach, heart, brain, liver, placenta, lung, skeletal muscle, or small intestine.

As TANGO 275 exhibits expression in the heart, TANGO 275 polypeptides, nucleic acids, or modulators thereof can be used to treat heart and cardiovascular disorders, such as ischemic heart disease as described herein.

In another example, TANGO 275 polypeptides, nucleic acids, or modulators thereof can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain (e.g., spinal cord injuries, infarction, infection, malignancy, exposure to toxic agents, nutritional deficiency, paraneoplastic syndromes), degenerative nerve diseases (including but not limited to Alzheimer's disease, Parkinson's disease, Huntington's Chorea, Gilles de la Tourette's syndrome, amyotrophic lateral sclerosis, progressive supra-nuclear palsy, and other dementias), and neuropsychiatric disorders (including schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective disorder, bipolar affective disorder with hypomania and major depression).

In another example, TANGO 275 polypeptides, nucleic acids, or modulators thereof can be used to treat placental disorders, such as toxemia of pregnancy (e.g., preeclampsia and eclampsia), placentitis, or spontaneous abortion.

In another example, TANGO 275 polypeptides, nucleic acids, or modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchiil carcinoid, hamartoma, and mesenchymal tumors).

In another example, TANGO 275 polypeptides, nucleic acids, or modulators thereof can be used to treat hepatic disorders, such as jaundice, hepatic failure, liver cysts, chronic liver disease, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 275 polypeptides, nucleic acids, or modulators thereof can be used to treat disorders of skeletal muscle, such as muscular dystrophy (e.g., Duchenne muscular dystrophy, Becker Muscular Dystrophy, Emery-Dreifuss muscular dystrophy, Limb-Girdle muscular dystrophy, Facioscapulohumeral muscular dystrophy, myotonic dystrophy, oculopharyngeal muscular dystrophy, distal muscular dystrophy, and congenital muscular dystrophy), motor neuron diseases (e.g., amyotrophic lateral sclerosis, infantile progressive spinal muscular atrophy, intermediate spinal muscular atrophy, spinal bulbar muscular atrophy, and adult spinal muscular atrophy), myopathies (e.g., inflammatory myopathies (e.g., dermatomyositis and polymyositis), myotonia congenita, paramyotonia congenita, central core disease, nemaline myopathy, myotubular myopathy, and periodic paralysis), and metabolic diseases of muscle (e.g., phosphorylase deficiency, acid maltase deficiency, phosphofructokinase deficiency, Debrancher enzyme deficiency, mitochondrial myopathy, carnitine deficiency, carnitine palmityl transferase deficiency, phosphoglycerate kinase deficiency, phosphoglycerate mutase deficiency, lactate dehydrogenase deficiency, and myoadenylate deaminase deficiency).

In another example, TANGO 275 polypeptides, nucleic acids, or modulators thereof can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 275 polypeptides, nucleic acids, or modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

TANGO 275 includes an EGF-like domain. Proteins having such domains play a role in a wide variety of biological processes, including cholesterol uptake, blood coagulation, and specification of cell fate. Thus, TANGO 275 polypeptides, nucleic acids, and modulators thereof can be used modulate these processes. TANGO 275 polypeptides, nucleic acids, and modulators thereof can be used to modulate cell proliferation, morphogenesis, tissue repair and renewal, terminal differentiation, cell survival, and cell migration. They can be used to treat cancer, promote wound healing (e.g., of the skin, cornea, or mucosa), and modulate an allergic or inflammatory response.

TANGO 275 includes a TB domain. Proteins having this domain are commonly associated with extracellular matrix fibrils. TANGO 275 polypeptides, nucleic acids, and modulators thereof can be used to modulate matrix formation and degradation and to treat disorders of the connective tissue, e.g., Marfan syndrome.

As a transforming growth factor-β binding protein, TANGO 275 can interact with transforming growth factor-β (TGF-β). In general, transforming growth factor-β binding proteins (LTBP) bind to TGF-β to form latent growth factor complexes (large latent complexes). LTBP are important regulators of TGF-β activity. LTBP are thought to facilitate the normal assembly and secretion of large latent complexes, target latent TGF-β to certain connective tissues, modulate the activity of large latent complexes, and target latent TGF-β to the cell surface. Given that TANGO 275 can modulate TGF-β activity, TANGO 275 polypeptides, nucleic acids, and modulators of TANGO 275 expression or activity can be used to treat connective tissue and bone disorders such as bone fracture, osteoporosis, and osteogenesis imperfecta. In addition, such compounds can be used to promote bone repair, promote bone regeneration, and improve bone implant bonding. Thus, TANGO 275 polypeptides, nucleic acids, and modulators thereof can be used to modulate various aspects of bone repair and regeneration, including, e.g., clot formation, clot dissolution, removal of damaged tissue, growth of granulation tissue, cartilage growth and turnover, formation of callus tissue, remodeling, formation of trabecular bone, and formation of cortical bone.

Further, in light of TANGO 275's pattern of expression in humans, TANGO 275 expression can be utilized as a marker for specific tissues (e.g., cardiovascular tissue such as the heart) and/or cells (e.g., cardiac) in which TANGO 275 is expressed. TANGO 275 nucleic acids can also be utilized for chromosomal mapping.

### MANGO 245

A cDNA encoding MANGO 245 was identified by analyzing the sequences of clones present in a human adult brain cDNA library.

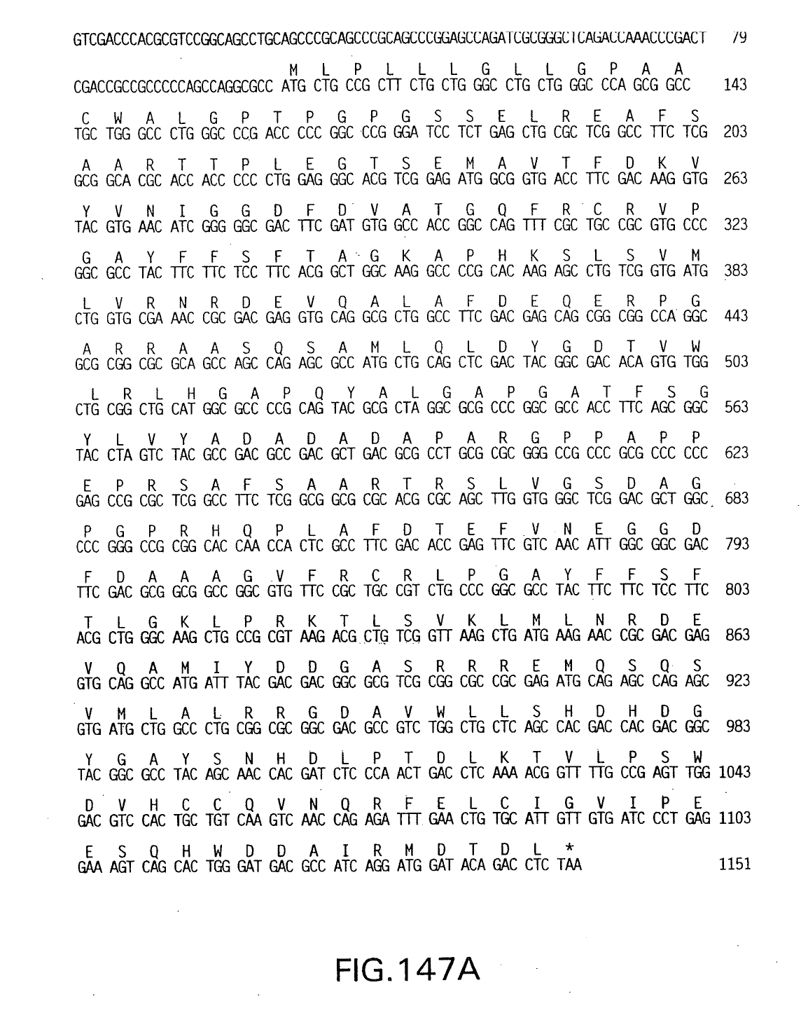

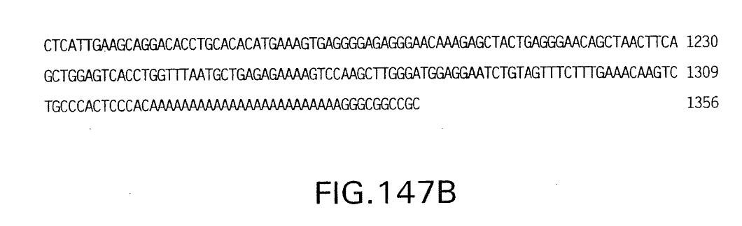

This analysis led to the identification of a clone, Alhbab165e5, encoding full-length human MANGO 245. The cDNA of this clone is 1356 nucleotides long (FIG. 147A-147B; SEQ ID NO:99). The 1044 nucleotide open reading frame of this cDNA, nucleotide 15105 to nucleotide 1148, encodes a 348 amino acid protein (SEQ ID NO: 100).

The signal peptide prediction program SIGNALP (Nielsen et al., 1997, Protein Engineering 10:1-6) predicted that human MANGO 245 includes a 16 amino acid signal peptide (amino acid 1 to about amino acid 16 of SEQ ID NO:100) preceding the mature human MANGO 245 protein (corresponding to about amino acid 17 to amino acid 348 of SEQ ID NO: 100).

Human MANGO 245 is a transmembrane protein having an extracellular domain which extends from about amino acid 17 to about amino acid 141, a transmembrane domain which extends from about amino acid 142 to about amino acid 158, and a cytoplasmic domain which extends from about amino acid 159 to amino acid 348 of SEQ ID NO:100.

Human MANGO 245 that has not been post-translationally modified is predicted to have a molecular weight of 37.9 kDa prior to cleavage of its signal peptide and a molecular weight of 36.3 kDa subsequent to cleavage of its signal peptide.

In one embodiment, a MANGO 245 protein contains a signal peptide of amino acids 1 to 16 (1 to 14, 1 to 15, 1 to 17, 1 to 18) of SEQ ID NO:94.

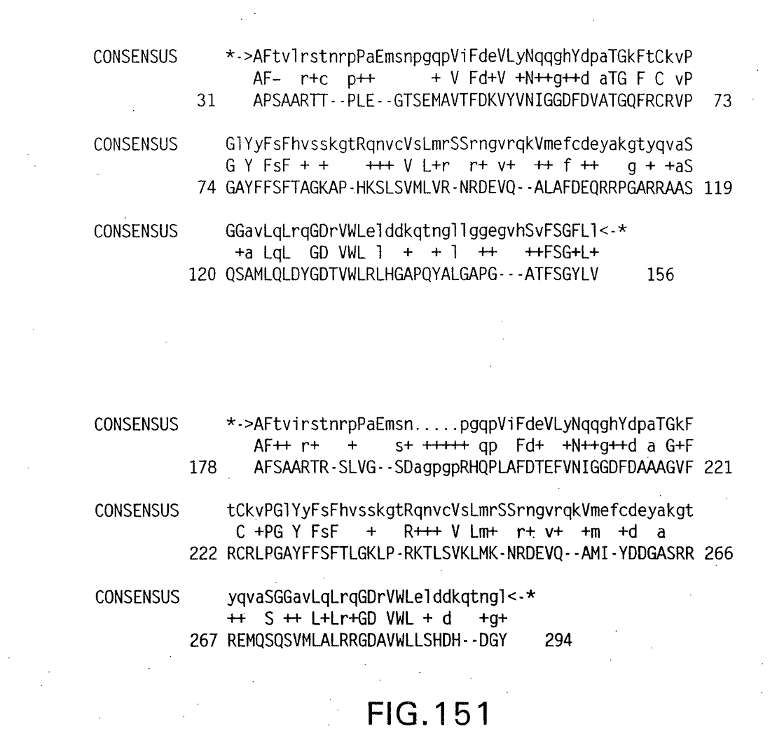

MANGO 245 family members can also include a CIq domain. A consensus hidden Markov model CIq domain has the amino acid sequence shown in the alignments depicted in FIG. 151 where the more conserved residues in the consensus sequence are indicated by uppercase letters and the less conserved residues in the consensus sequence are indicated by lowercase letters. Human MANGO 245 includes CIq domains at amino acids 31 to 156 and amino acids 178 to 294. Monkey MANGO 245 includes CIq domains at amino acids 31 to 156 and amino acids 178 to 311. Mouse MANGO 245 includes a CIq domain at amino acids 30 to 155. CIq domains are found in wholly secreted or membrane bound proteins that are short-chain collagens and collagen-like molecules. The domain likely forms ten β-strands interspersed by β-turns and/or loops.

Within MANGO 245, protein kinase C phosphorylation sites are present at amino acids 244 to 246 and 264 to 266. Casein kinase II phosphorylation sites are present at amino acids 38 to 41 and 298 to 301. N-myristylation sites are present at amino acids 66 to 71, 113 to 118, 145 to 150, 219 to 224, and 295 to 300.

Clone Alhbab165e5, which encodes human MANGO 245, was deposited as EpM245 with the American Type Culture Collection (ATCC® 10801 University Boulevard, Manassas, Va. 20110-2209) on Apr. 21, 1999 and assigned Accession Number 207223. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

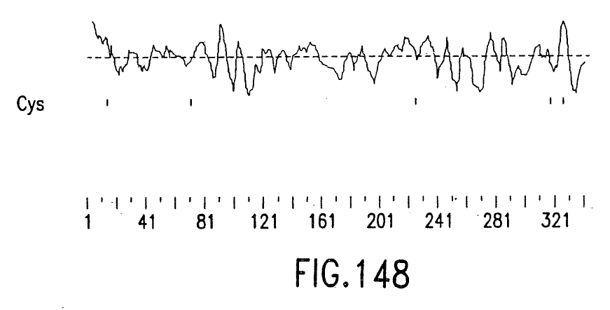

FIG. 148 depicts a hydropathy plot of human MANGO 245. The hydropathy plot indicates that human MANGO 245 has a signal peptide at its amino terminus and an internal hydrophobic region, suggesting that human MANGO 245 is a transmembrane protein.

Northern blot analysis of human MANGO 245 expression revealed that human MANGO 245 is expressed at a relatively high level in the cerebellum, frontal lobe, and putamen; at a moderate level in the cerebral cortex, the medulla, occipital lobe, and temporal lobe; and a relatively low level in the spinal cord. Additional Northern blot analysis revealed the human MANGO 245 is expressed in amygdala, caudate nucleus, hippocampus, brain, substantia nigra, and subthalamic nucleus.

A cDNA encoding monkey MANGO 245 was identified by analyzing the sequences of clones present in a monkey cDNA library.

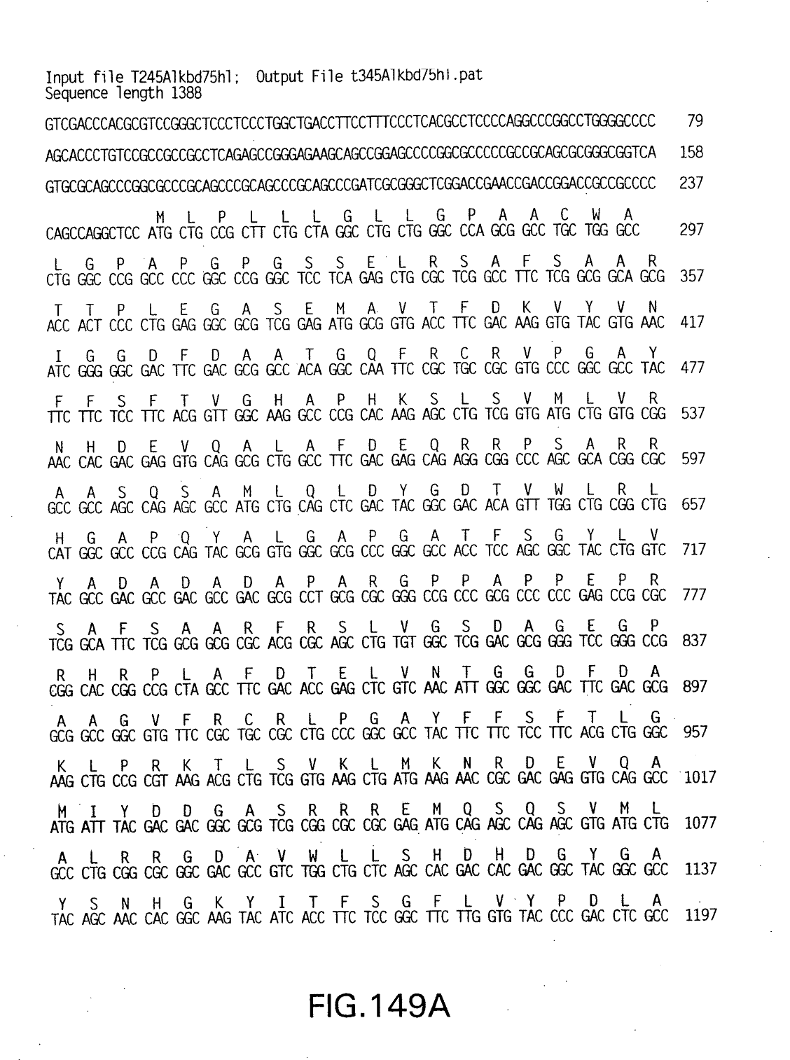

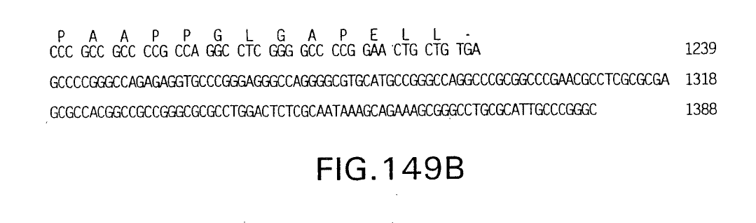

This analysis led to the identification of a clone, Alkbd75h1, encoding full-length monkey MANGO 245. The cDNA of this clone is 1416 nucleotides long (FIG. 149A-149B; SEQ ID NO:101). The 987 nucleotide open reading frame of this cDNA, nucleotide 250 to nucleotide 1236, encodes a 329 amino acid protein (SEQ ID NO: 102).

The signal peptide prediction program SIGNALP (Nielsen et al., 1997, Protein Engineering 10:1-6) predicted that monkey MANGO 245 includes a 16 amino acid signal peptide (amino acid 1 to about amino acid 16 of SEQ ID NO: 102) preceding the mature monkey MANGO 245 protein (corresponding to about amino acid 17 to amino acid 329 of SEQ ID NO:102).

Monkey MANGO 245 that has not been post-translationally modified is predicted to have a molecular weight of 35.2 kDa prior to cleavage of its signal peptide and a molecular weight of 33.6 kDa subsequent to cleavage of its signal peptide.

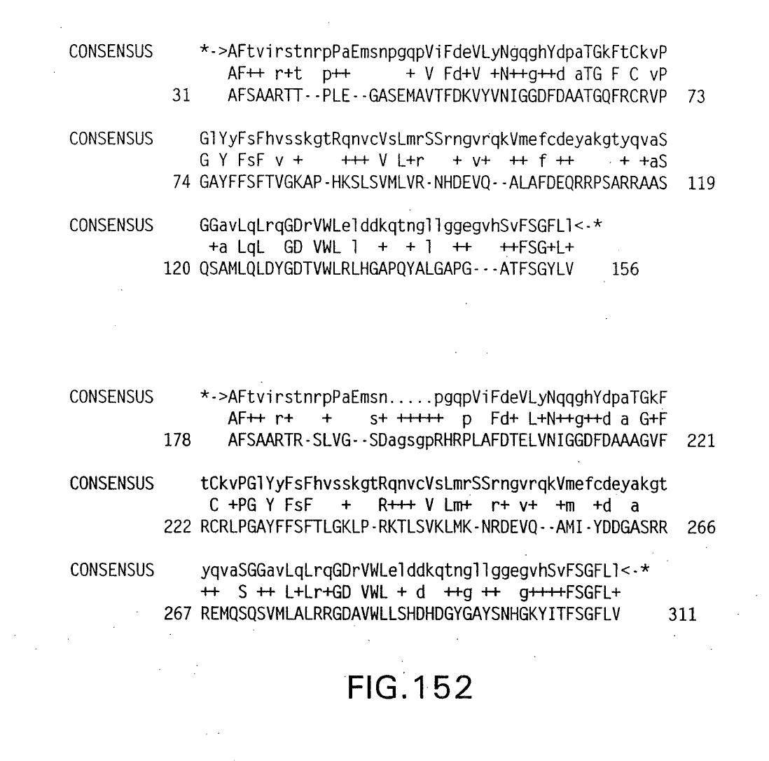

Monkey MANGO 245 includes CIq domains at amino acids 31 to 156 and amino acids 178 to 311 of SEQ ID NO:102. FIG. 152 depicts alignments of the CIq domains of monkey MANGO 245 with a consensus hidden Markov model CIq domain.

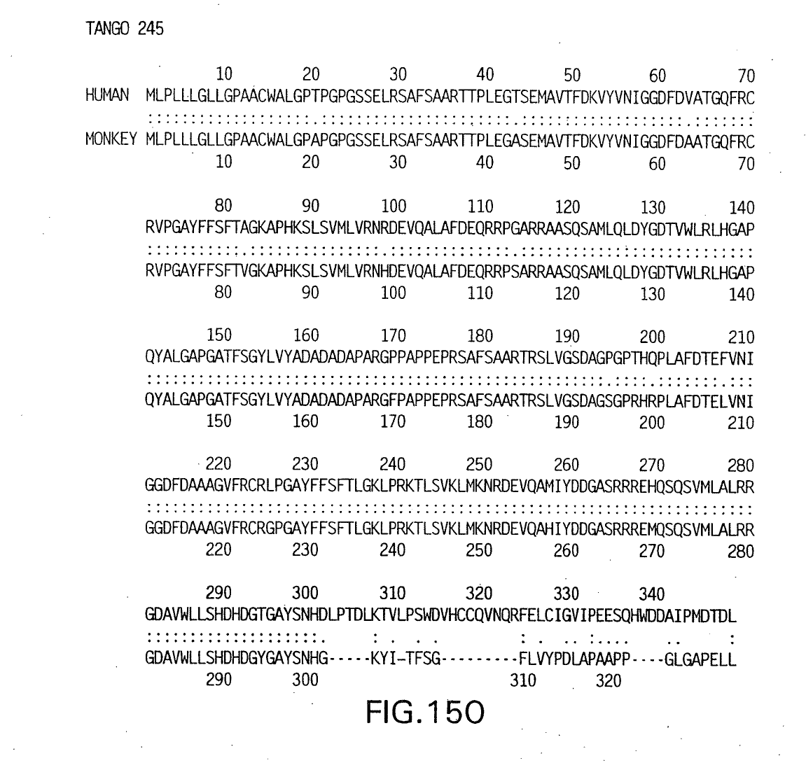

FIG. 150 depicts an alignment of the amino acid sequence of human MANGO 245 and the amino acid sequence of monkey MANGO 245. This alignment was created using ALIGN (version 2.0; PAM120 scoring matrix; gap length penalty of 12; gap penalty of 4). In this alignment, the sequences are 84.8% identical overall.

Clone Atkbd75h1, which encodes monkey MANGO 245, was deposited as EpK245 with the American Type Culture Collection (ATCC® 10801 University Boulevard, Manassas, Va. 20110-2209) on Jun. 18, 1999 and assigned Accession Number PTA-248. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

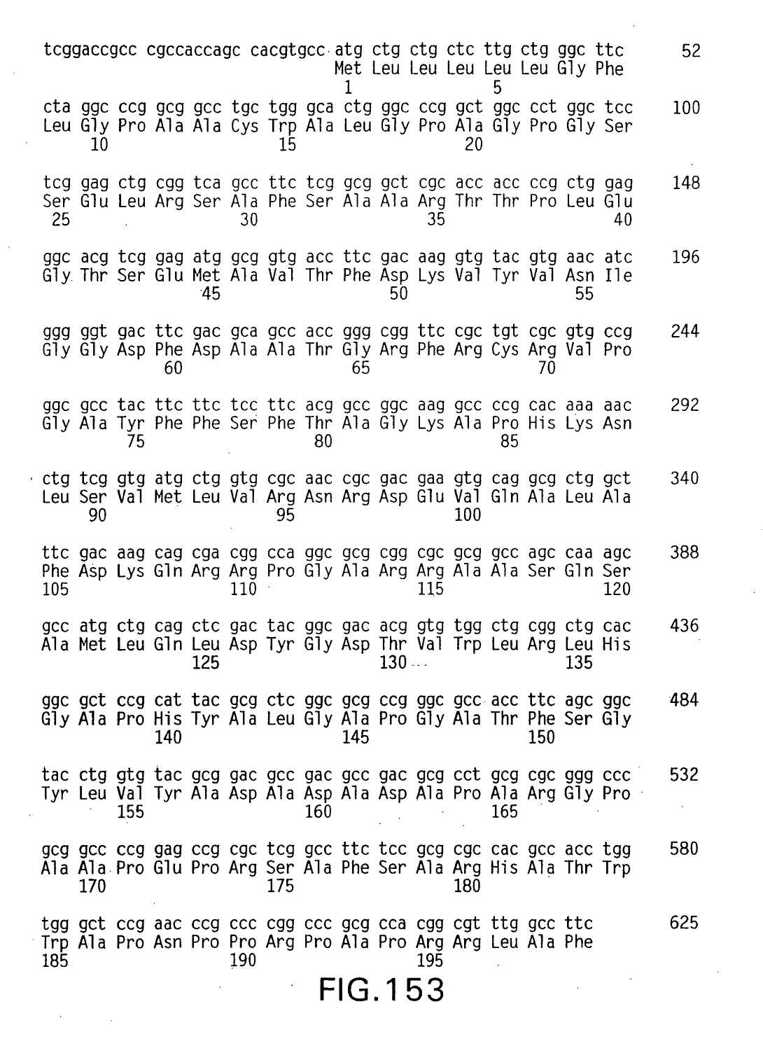

In addition, a mouse MANGO 245 was identified. The cDNA of this clone is 625 nucleotides long (FIG. 153; SEQ ID NO: 103). The open reading frame of this cDNA is begins at nucleotide 29. Mouse MANGO 245 includes a CIq domain at amino acids 30 to 155 of SEQ ID NO:104.

Within mouse MANGO 245, protein kinase C phosphorylation sites are present at amino acids 64 to 66 and 178 to 180. N-myristylation sites are present at amino acids 112 to 117 and 144 to 149. A casein kinase II phosphorylation site is present at amino acids 37 to 40. An N-glycosylation site is present at amino acids 88 to 91.

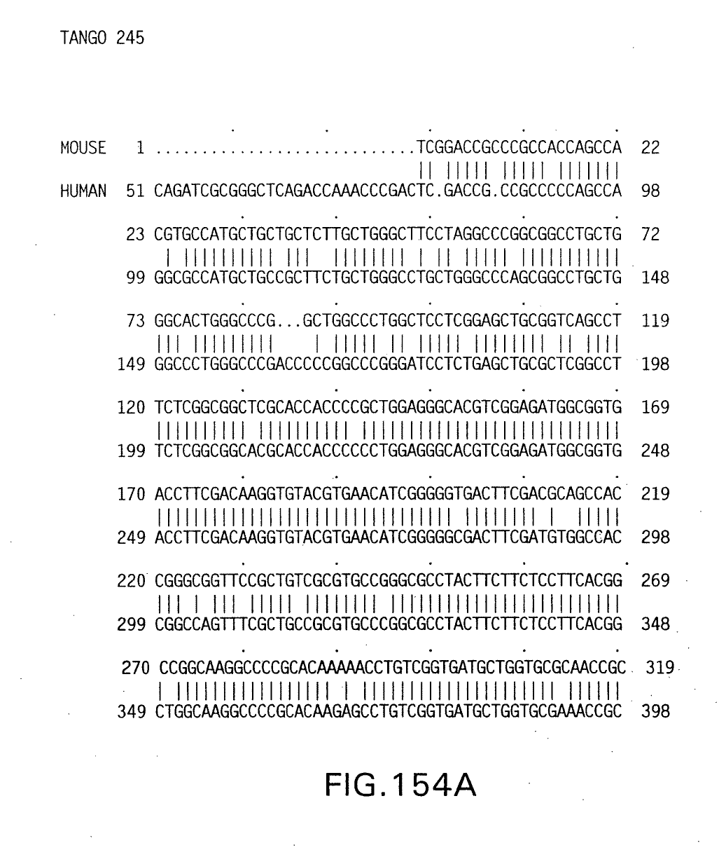

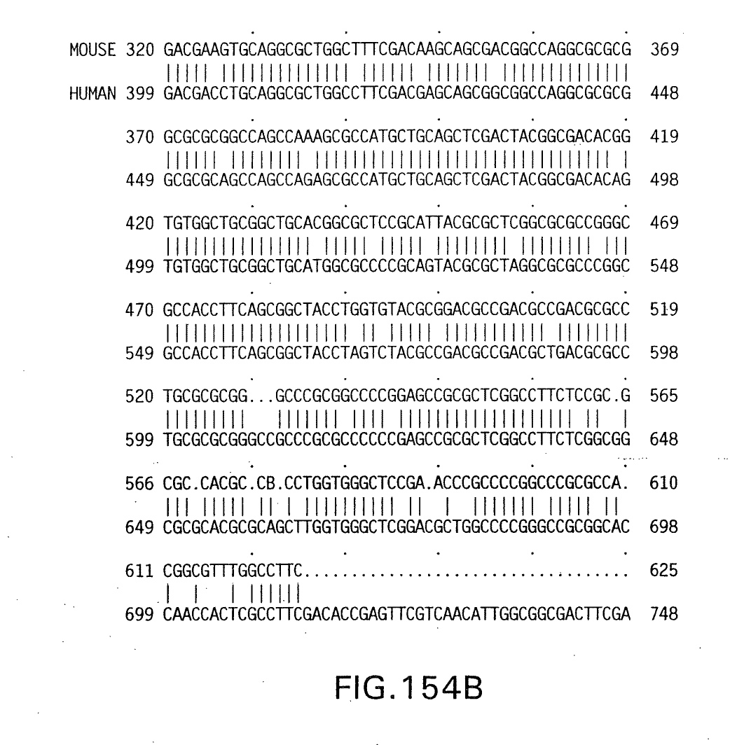

FIG. 154A-154B depicts an alignment of 697 of the 1356 nucleotides of the human MANGO 245 sequence (nucleotide 51 to nucleotide 748 of SEQ ID NO:99) with the nucleotide sequence of mouse MANGO 245. This alignment was created using BESTFIT (BLOSUM 62 scoring matrix; gap open penalty of 12; frame shift penalty of 5; gap extend penalty of 4). In this alignment, the sequences are 89.6% identical overall.

### Use of MANGO 245 Nucleic Acids, Polypeptides, and Modulators Thereof

MANGO 245 polypeptides, nucleic acids, and modulators thereof can be used to modulate the function, morphology, proliferation and/or differentiation of cells in the tissues in which they are expressed. MANGO 245 is expressed in the brain and central nervous system. Thus, MANGO 245 polypeptides, nucleic acids, and modulators thereof can be used to treat CNS disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders, and neuropsychiatric disorders that include, but are not limited to schizophrenia, schizoaffective disorder, attention deficit disorder, dysthymic disorder, major depressive disorder, mania, obsessive-compulsive disorder, psychoactive substance use disorders, anxiety, panic disorder, as well as bipolar affective disorder, e.g., severe bipolar affective (mood) disorder (BP-I), bipolar affective (mood) disorder with hypomania and major depression (BP-II).

MANGO 245 includes a CIq domain. Known proteins having this domain play a role complement activation and autoimmune disorders. The CIq domain is also found in collagens and collagen-like molecules. MANGO 245 polypeptides, nucleic acids, and modulators thereof can be used to treat disorders of collagen assembly and degradation.

Further, in light of MANGO 245's pattern of expression in humans, MANGO 245 expression can be utilized as a marker for specific tissues (e.g., brain) and/or cells (e.g., cerebellum, frontal lobe, or putamen) in which MANGO 245 is expressed. MANGO 245 nucleic acids can also be utilized for chromosomal mapping.

### INTERCEPT 340

A cDNA encoding INTERCEPT 340 was identified by analyzing the sequences of clones present in a human fetal spleen cDNA library.

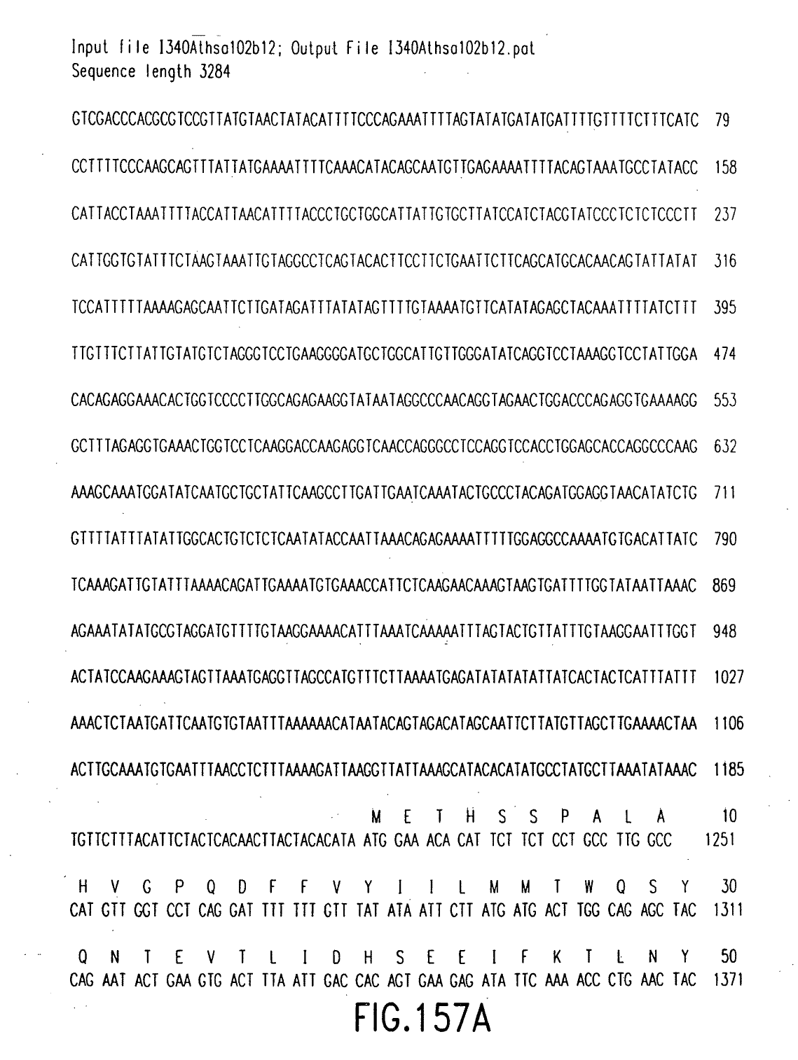

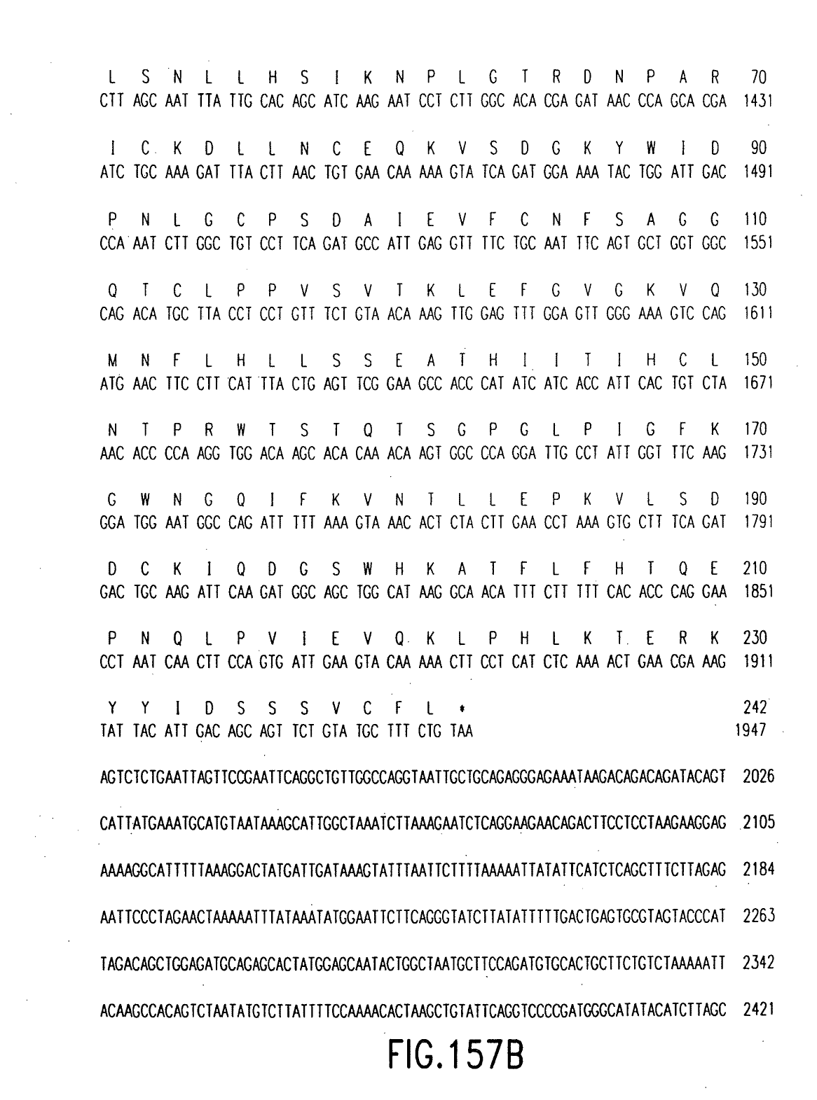

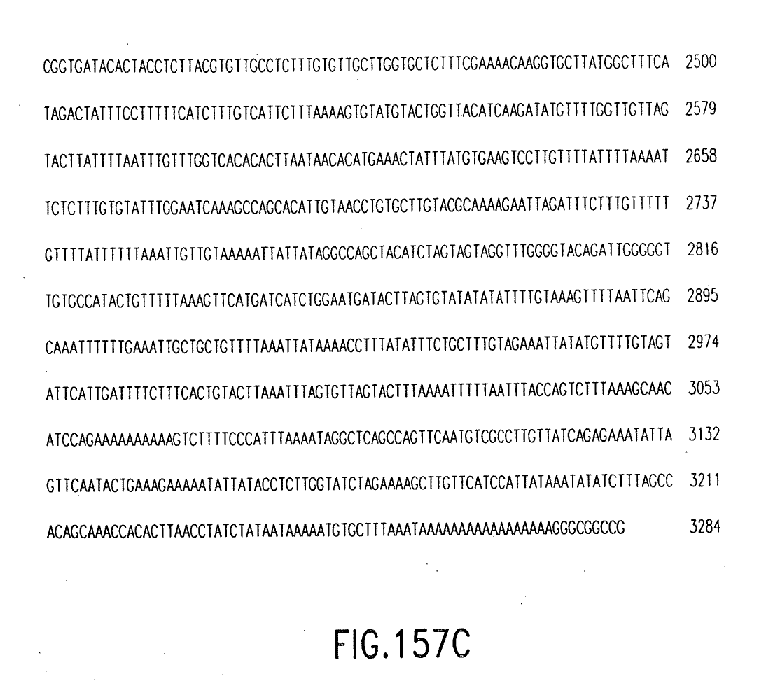

This analysis led to the identification of a clone, jthsa102b12, encoding full-length human INTERCEPT 340. The cDNA of this clone is 3284 nucleotides long (FIG. 157A-157C; SEQ ID NO:105). The 723 nucleotide open reading frame of this cDNA (nucleotides 1222-1944 of SEQ ID NO:105) encodes a 241 amino acid protein (SEQ ID NO:106).

Human INTERCEPT 340 that has not been post-translationally modified is predicted to have a molecular weight of 27.2 kDa.

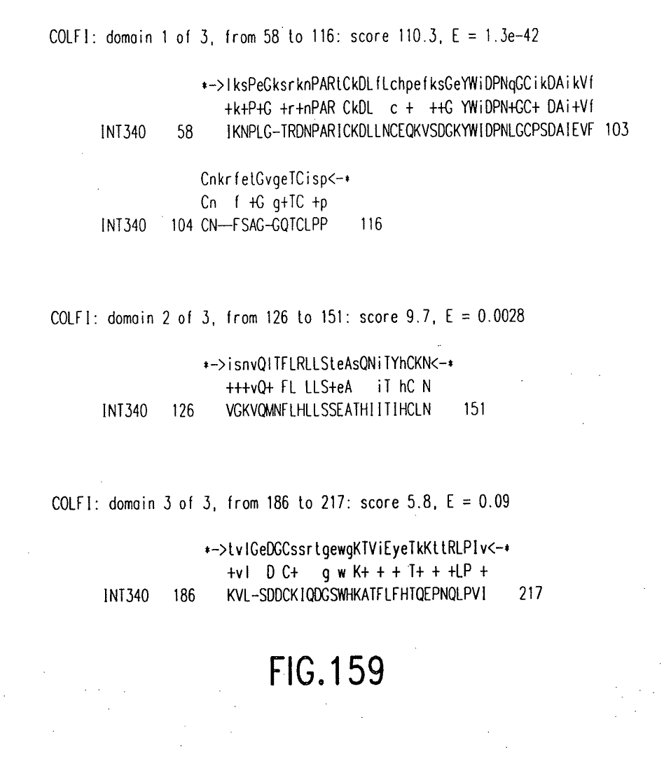

INTERCEPT 340 family members can include at least one, preferably two, and more preferably three fibrillar collagen C-terminal domains (also referred to herein as “COLF domains”). As used herein, a “fibrillar collagen C-terminal domain” refers to an amino acid sequence of about 15 to 65, preferably about 20-60, more preferably about 25, 31-58 amino acids in length. Consensus hidden Markov model COLF domains are depicted in FIG. 159. The more conserved residues in the consensus sequence are indicated by uppercase letters and the less conserved residues in the consensus sequence are indicated by lowercase letters. A comparison of the C-terminal sequences of fibrillar collagens, collagens X, VIII, and the collagen C1q revealed a conserved cluster of amino acid residues having aromatic side chains (e.g., tyrosine, phenylalanine, tryptophan, histidine) that exhibited marked similarities in hydrophilicity profiles between the different collagens, despite a low level of sequence similarity. These similarities in hydrophilicity profiles within their C-termini suggest that these proteins may adopt a common tertiary structure and that the conserved cluster of aromatic residues in this domain may be involved in C-terminal trimerization. The COLF domains of INTERCEPT 340 extend from about amino acids 58 to 116, 126 to 151, and 186 to 217 (FIG. 159). By alignment of the amino acid sequence of the consensus hidden Markov model COLF amino acid sequence with the amino acid sequence of the COLF domains of INTERCEPT 340, conserved amino acid residues having aromatic side chains can be found. For example, conserved tyrosine, tryptophan and phenylalanine residues can be found at amino acid 87, 88 and 133.

Human INTERCEPT 340 includes three fibrillar collagen C-terminal (COLF) domains at amino acids 58-116; amino acids 126-151; and amino acids 186-217 of SEQ ID NO:106. FIG. 159 depicts alignments of each of the COLF domains of human INTERCEPT 340 with consensus hidden Markov model COLF domains. In one embodiment, INTERCEPT 340 is a secreted protein. In another embodiment, INTERCEPT 340 is a membrane-associated protein.

An N-glycosylation site is present at amino acids 105-108. A glycosaminoaglycan attachment site is present at amino acids 161-164. Protein kinase C phosphorylation sites are present at amino acids 57-59, 152-154, and 227-229. A tyrosine kinase phosphorylation site is present at amino acids 81-87. Casein kinase II phosphorylation sites are present at amino acids 36-39, 120-123 and 181-184. N-myristylation sites are present at amino acids 109-114 and 164-169.

Clone jthsa102b12, which encodes human INTERCEPT 340, was deposited as a composite deposit having a designation EpI340 with the American Type Culture Collection (ATCC® 10801 University Boulevard, Manassas, Va. 20110-2209) on Jun. 18, 1999 and assigned Accession Number PTA-250. A description of the deposit conditions is set forth in the section entitled “Deposit of Clones” below. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

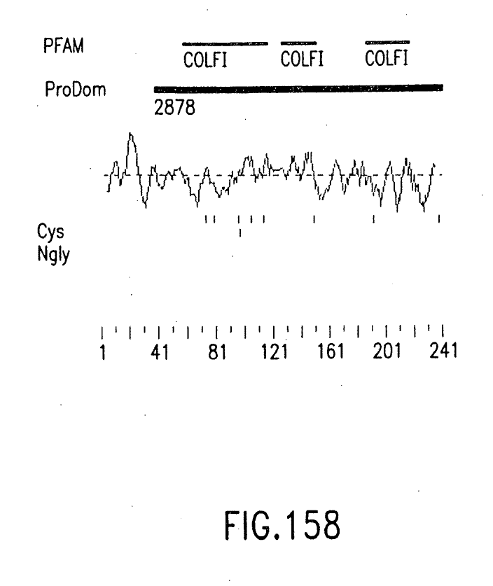

FIG. 158 depicts a hydropathy plot of human INTERCEPT 340.

### Use of INTERCEPT 340 Nucleic Acids, Polypeptides, and Modulators Thereof

INTERCEPT 340 includes three fibrillar collagen C-terminal domains. Proteins having such domains play a role in modulating connective tissue formation and/or maintenance, and thus can influence a wide variety of biological processes, including assembly into fibrils; strengthening and organization of the extracellular matrix; shaping of tissues and cells; modulation of cell migration; and/or modulation of signal transduction pathways. Because INTERCEPT 340 includes fibrillar collagen C-terminal domains, INTERCEPT 340 polypeptides, nucleic acids, and modulators thereof can be used to treat connective tissue disorders, including a skin disorder and/or a skeletal disorder (e.g., Marfan syndrome and osteogenesis imperfecta); cardiovascular disorders including hyperproliferative vascular diseases (e.g., hypertension, vascular restenosis and atherosclerosis), ischemia reperfusion injury, cardiac hypertrophy, coronary artery disease, myocardial infarction, arrhythmia, cardiomyopathies, and congestive heart failure); and/or hematopoietic disorders (e.g., myeloid disorders, lymphoid malignancies, T cell disorders).

As INTERCEPT 340 was originally found in a fetal spleen library, INTERCEPT 340 nucleic acids, proteins, and modulators thereof can be used to modulate the function, survival, morphology, migration, proliferation and/or differentiation of cells that form the spleen, e.g., cells of the splenic connective tissue, e.g., splenic smooth muscle cells and/or endothelial cells of the splenic blood vessels. INTERCEPT 340 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus INTERCEPT 340 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

Further, in light of INTERCEPT 340's presence in a human fetal spleen cDNA library, INTERCEPT 340 expression can be utilized as a marker for specific tissues (e.g., lymphoid tissues such as the spleen) and/or cells (e.g., splenic) in which INTERCEPT 340 is expressed. INTERCEPT 340 nucleic acids can also be utilized for chromosomal mapping.

### MANGO 003

A cDNA encoding human MANGO 003 was identified by analyzing the sequences of clones present in a human thyroid cDNA library.

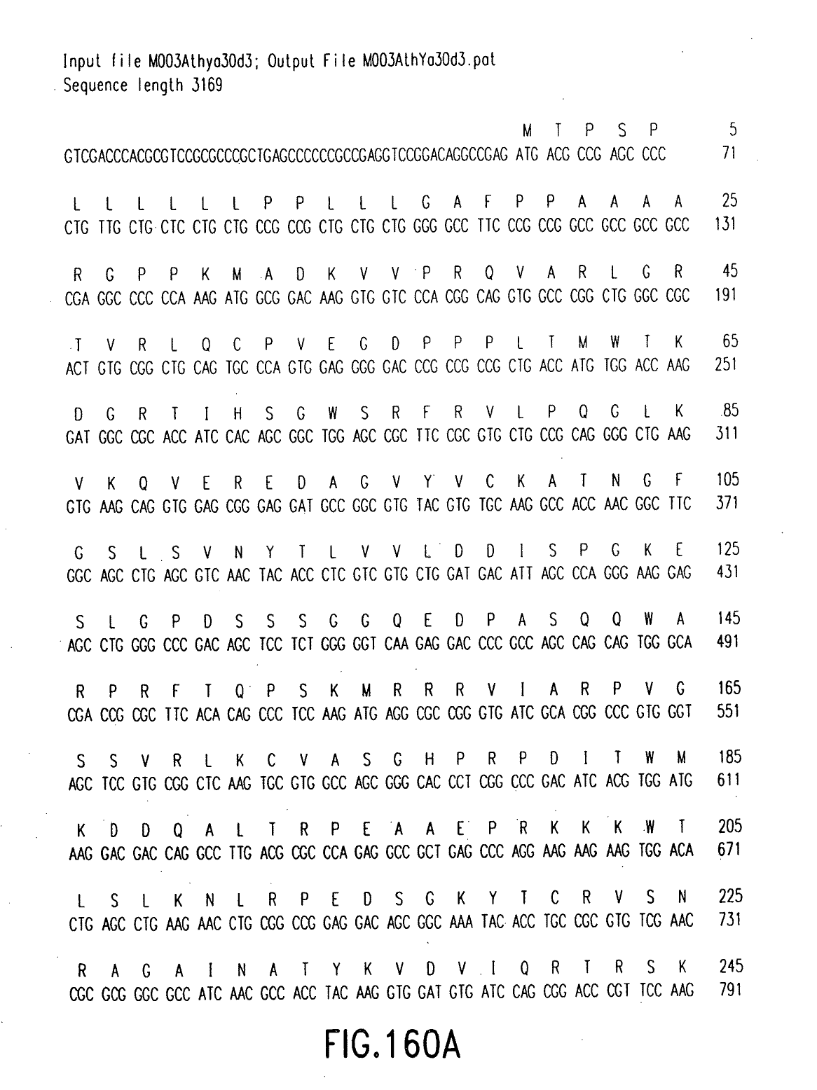

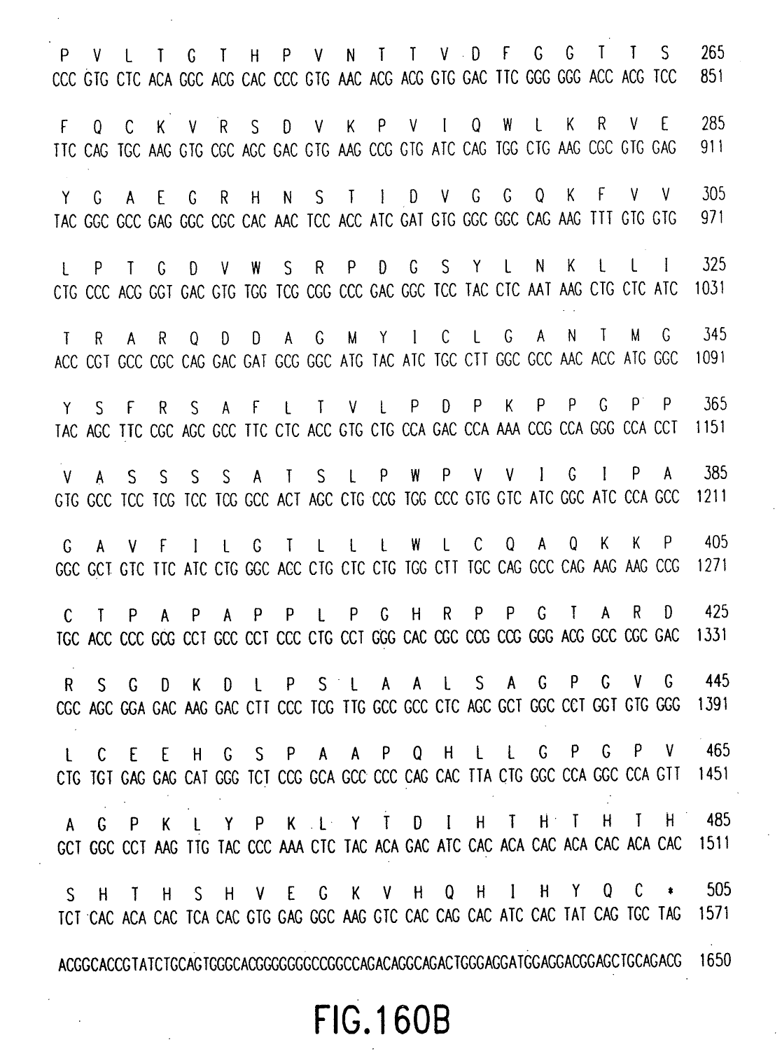

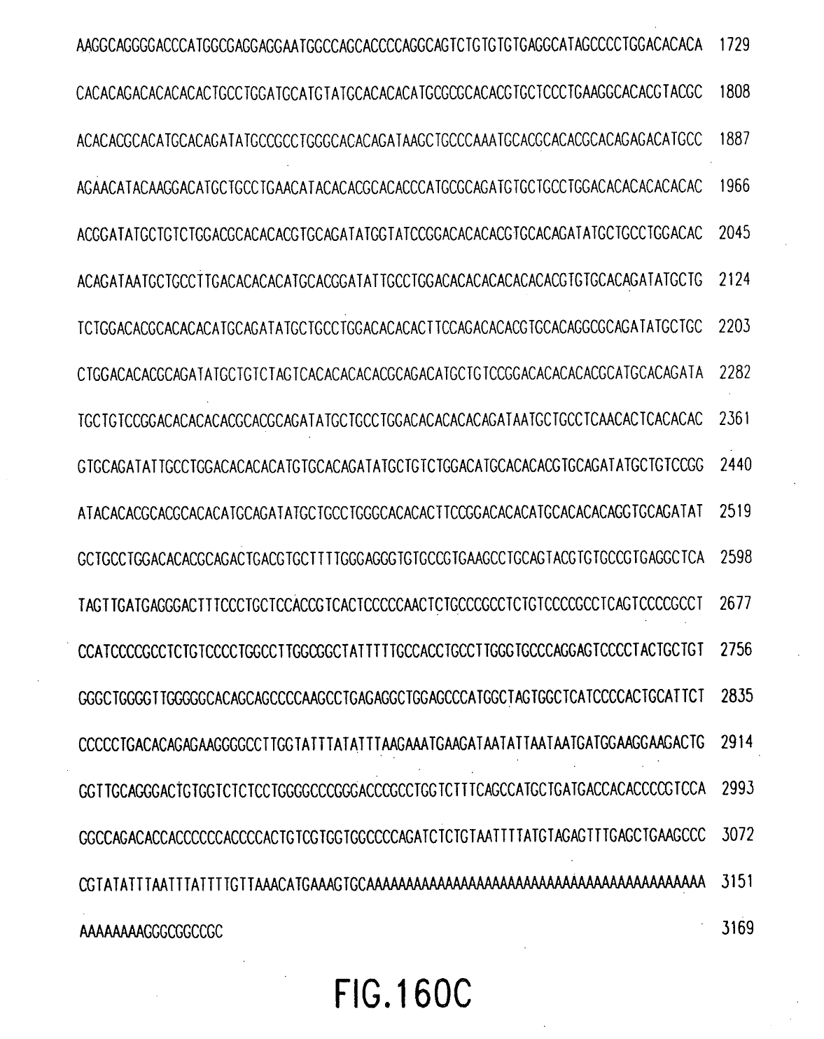

This analysis led to the identification of a clone, jthYa030d03, encoding full-length human MANGO 003. The cDNA of this clone is 3169 nucleotides long (FIG. 160A-160C; SEQ ID NO: 107). The 1512 nucleotide open reading frame of this cDNA (nucleotide 57 to nucleotide 1568 of SEQ ID NO: 107) encodes a 504 amino acid protein (SEQ ID NO:108).

Human MANGO 003 that has not been post-translationally modified is predicted to have a molecular weight of 54.5 kDa prior to cleavage of its signal peptide (52.1 kDa after cleavage of its signal peptide).

The signal peptide prediction program SIGNALP (Nielsen et al., 1997, Protein Engineering 10:1-6) predicted that human MANGO 003 includes a 24 amino acid signal peptide at amino acid 1 to about amino acid 24 preceding the mature human MANGO 003 protein which corresponds to about amino acid 25 to amino acid 504 of SEQ ID NO: 108.

Human MANGO 003 is a transmembrane protein having an extracellular domain which extends from about amino acid 25 to about amino acid 374, a transmembrane domain which extends from about amino acid 375 to about amino acid 398, and a cytoplasmic domain which extends from about amino acid 399 to amino acid 504 of SEQ ID NO: 108.

Alternatively, in another embodiment, a human MANGO 003 protein contains an extracellular domain which extends from about amino acid 399 to amino acid 504, a transmembrane domain which extends from about amino acid 375 to about amino acid 398, and a cytoplasmic domain which extends from about amino acid 25 to about amino acid 374 of SEQ ID NO: 108.

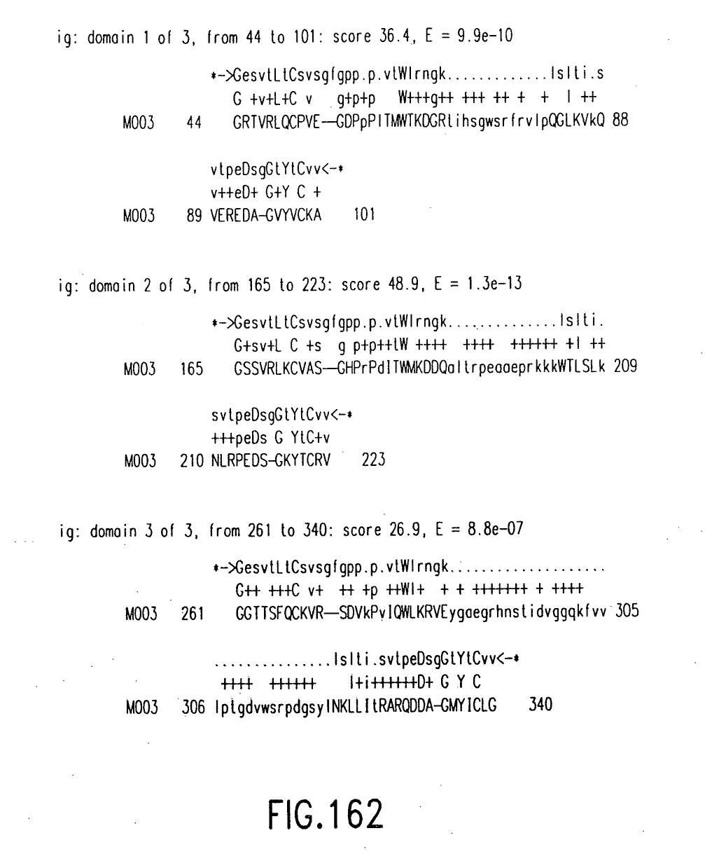

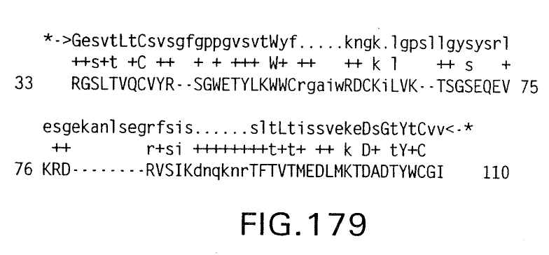

MANGO 003 family members can include at least one, preferably two, and more preferably three immunoglobulin domains. As used herein, an “immunoglobulin domain” (also referred to herein as “Ig”) refers to an amino acid sequence of about 45 to 85, preferably about 55-80, more preferably about 57, 58, or 78, 79 amino acids in length. Preferably, the immunoglobulin domains have a bit score for the alignment of the sequence to the Ig family Hidden Markov Model (HMM) of at least 10, preferably 20-30, more preferably 22-40, more preferably 40-50, 50-75, 75-100, 100-200 or greater. The Ig family HMM has been assigned the PFAM Accession PF00047. Consensus hidden Markov model immunoglobulin domains are shown FIGS. 162 and 179. The more conserved residues in the consensus sequence are indicated by uppercase letters and the less conserved residues in the consensus sequence are indicated by lowercase letters. Immunoglobulin domains are present in a variety of proteins (including secreted and membrane-associated proteins). Membrane-associated proteins may be involved in protein-protein, and protein-ligand interaction at the cell surface, and thus may influence diverse activities including cell surface recognition and/or signal transduction. The immunoglobulin domains of MANGO 003 extend from about amino acids 44 to 101, 165 to 223, and 261 to 240 (FIG. 162). The immunoglobulin domain of TANGO 354 extend from about amino acids 33 to 110 (FIG. 179).

Human MANGO 003 includes three immunoglobulin domains at amino acids 44-101; amino acids 165-223; and amino acids 261-340 of SEQ ID NO:108. FIG. 162 depicts alignments of each of the immunoglobulin domains of MANGO 003 with a consensus hidden Markov model immunoglobulin domain.

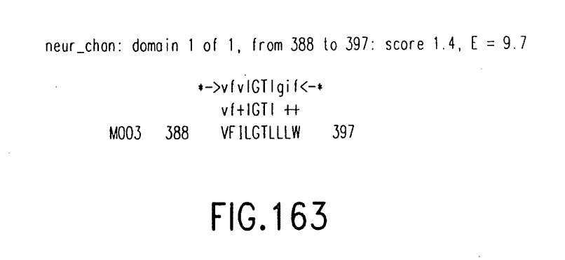

MANGO 003 family member can include a neurotransmitter-gated ion channel domain. As used herein, a “neurotransmitter-gated ion channel domain” refers to an amino acid sequence of about 5 to 20, preferably about 7 to 12, more preferably about 9 to 10 amino acids in length. The neurotransmitter-gated ion channel domain HMM has been assigned the PFAM Accession PF00065. A consensus hidden Markov model neurotransmitter-gated ion channel domain contain the sequence shown in FIG. 163. The more conserved residues in the consensus sequence are indicated by uppercase letters and the less conserved residues in the consensus sequence are indicated by lowercase letters. The neurotransmitter-gated ion channel domains of MANGO 003 extend from about amino acids 388 to 397 of SEQ ID NO:108.

In one embodiment, a MANGO 003 family member includes three immunoglobulin domains and a neurotransmitter-gated ion channel domain. In another embodiment, a MANGO 003 family member includes three immunoglobulin domains, a neurotransmitter-gated ion channel domain and a transmembrane domain. In yet another embodiment, a MANGO 003 family member includes three immunoglobulin domains, a neurotransmitter-gated ion channel domain, a transmembrane domain and an N-terminal extracellular domain.

In another embodiment, a MANGO 003 family member includes three immunoglobulin domains, a neurotransmitter-gated ion channel domain, a transmembrane domain, an N-terminal extracellular domain and a C-terminal cytoplasmic domain. In yet another embodiment, a MANGO 003 family member includes three immunoglobulin domains, a neurotransmitter-gated ion channel domain, a transmembrane domain, an N-terminal extracellular domain, a C-terminal cytoplasmic domain, and a signal peptide.

Human MANGO 003 includes a neurotransmitter gated ion channel domain at amino acids 388-397 of SEQ ID NO: 108. FIG. 163 depicts an alignment of the neurotransmitter gated ion channel domain of human MANGO 003 with a neurotransmitter gated ion channel domain derived from a hidden Markov model.

N-glycosylation sites are present at amino acids 111-114, 231-234, 255-258, and 293-296. A cAMP and cGMP-dependent protein kinase phosphorylation site is present at amino acids 202-205. Protein kinase C phosphorylation sites are present at amino acids 44-48, 167-169, 207-209, 216-218, 220-222, 224-226, 233-235, 347-349, and 422-424. Casein kinase II phosphorylation sites are present at amino acids 192-195, 256-259, 294-297, 313-316, 422-425, and 490-493. Tyrosine kinase phosphorylation sites are present at amino acids 212-219 and 329-336. N-myristylation sites are present at amino acids 95-100, 228-233, 261-266, 317-322, 334-339, 382-387, and 443-448.

Clone jthYa030d03, which encodes human MANGO 003, was deposited as a composite deposit having a designation EpthLa6a1 with the American Type Culture Collection (ATCC® 10801 University Boulevard, Manassas, Va. 20110-2209) on Mar. 27, 1999 and assigned Accession Number 207178. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

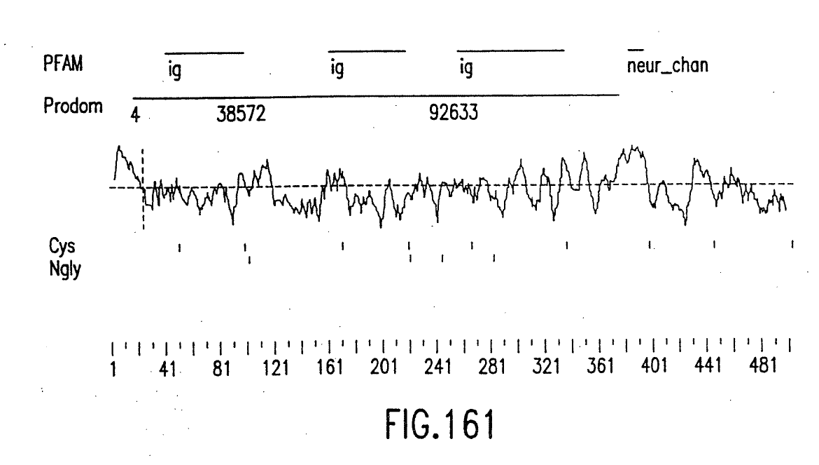

FIG. 161 depicts a hydropathy plot of human MANGO 003. The hydropathy plot indicates the presence of a hydrophobic domain within human MANGO 003, suggesting that human MANGO 003 is a transmembrane protein.

A cDNA encoding mouse MANGO 003 was identified by analyzing the sequences of clones present in a mouse choroid plexus cDNA library.

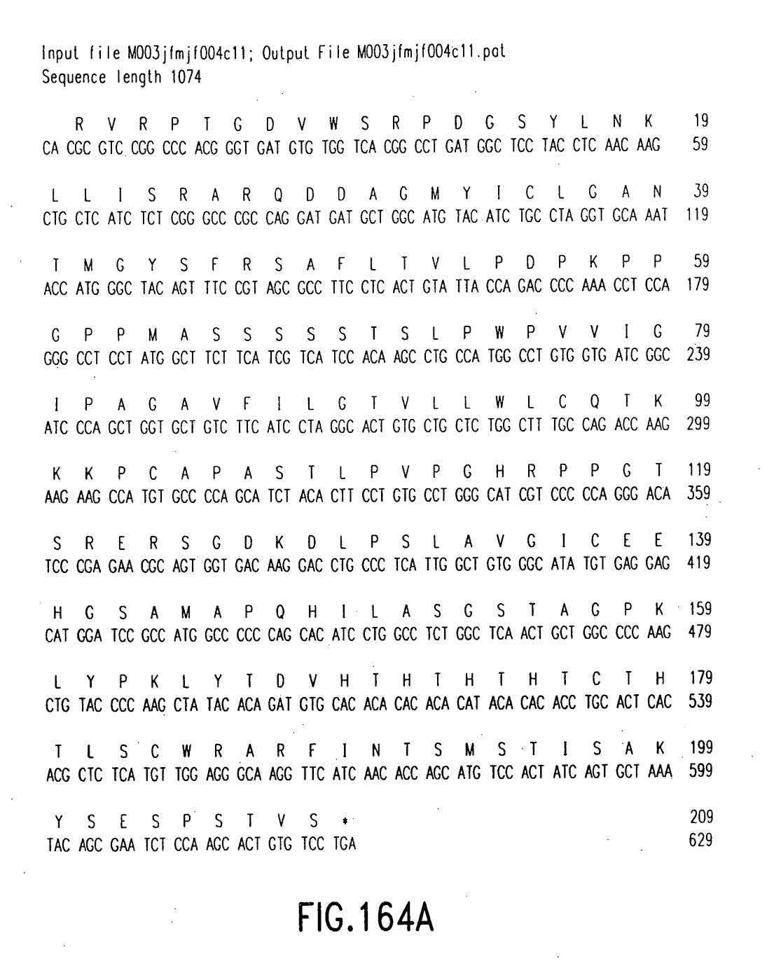

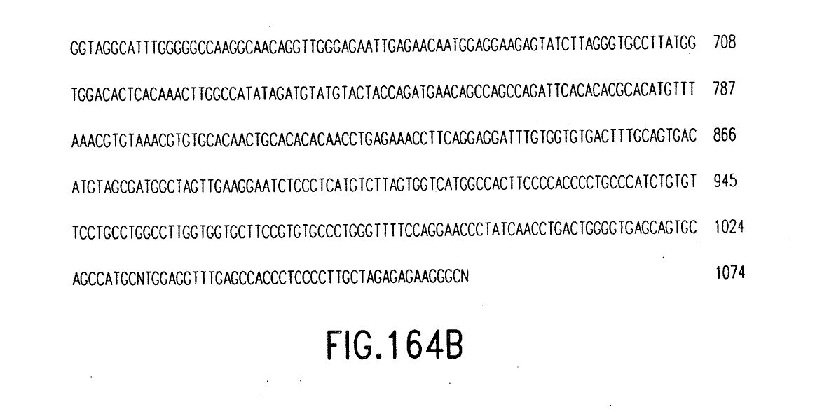

This analysis led to the identification of a clone, jfmjf004c11, encoding partial mouse MANGO 003. The cDNA of this clone is 626 nucleotides long (FIG. 164A-164B; SEQ ID NO: 109). The 626 nucleotide open reading frame of this cDNA, nucleotides 1-626, encodes a 208 amino acid protein (SEQ ID NO: 110).

Northern blot analysis using the mouse clone jfmjf004c11 revealed strong expression of the mouse MANGO 003 gene in the mouse liver, skeletal muscle and kidney. Moderate expression was detected in the heart, lung and testis, and lower levels of expression were detected in the mouse brain. No expression was detected in the spleen.

Mouse MANGO 003 that has not been post-translationally modified is predicted to have a molecular weight of 22.3 kDa.

Mouse MANGO 003 is a transmembrane protein having an extracellular domain which extends from about amino acid 1 to about amino acid 73, a transmembrane domain which extends from about amino acid 74 to about amino acid 96, and a cytoplasmic domain which extends from about amino acid 97 to amino acid 208 of SEQ ID NO: 110.

An N-glycosylation site is present at amino acids 190-193. Protein kinase C phosphorylation sites are present at amino acids 44-46, 98-100, 119-121, and 197-199. Casein kinase II phosphorylation sites are present at amino acids 10-13, and 119-122. A tyrosine kinase phosphorylation site is present at amino acids 26-33. N-myristylation sites are present at amino acids 14-19, 31-36, and 79-84.

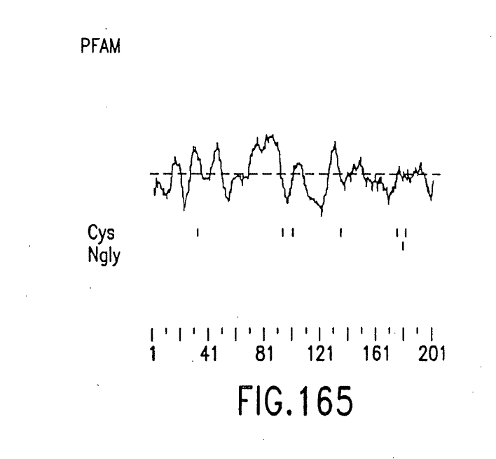

FIG. 165 depicts a hydropathy plot of mouse MANGO 003. The hydropathy plot indicates the presence of a hydrophobic domain within human MANGO 003, suggesting that human MANGO 003 is a transmembrane protein.

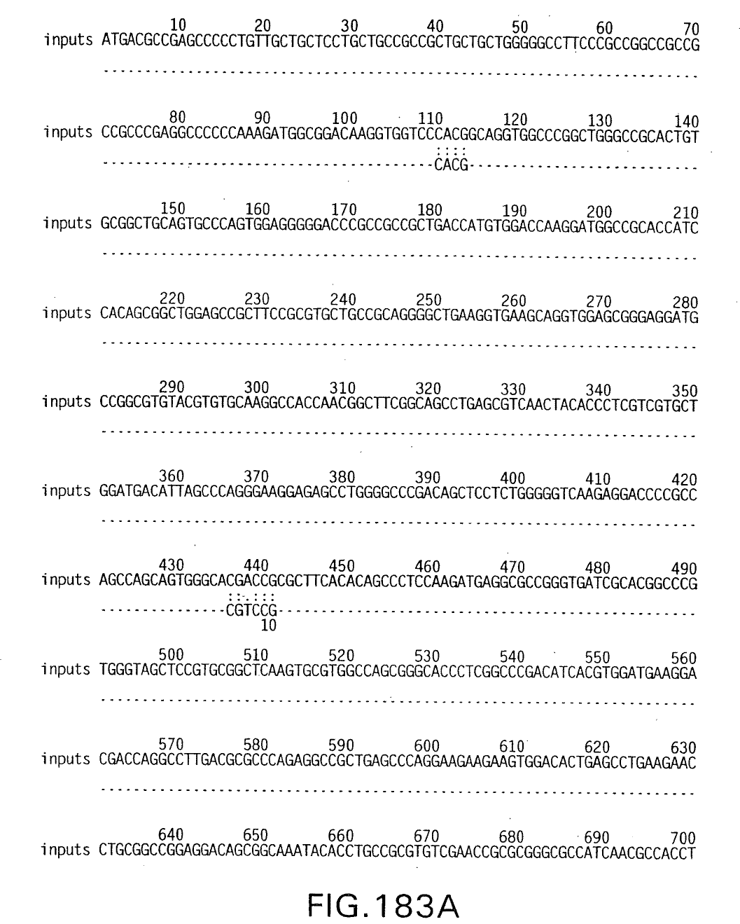

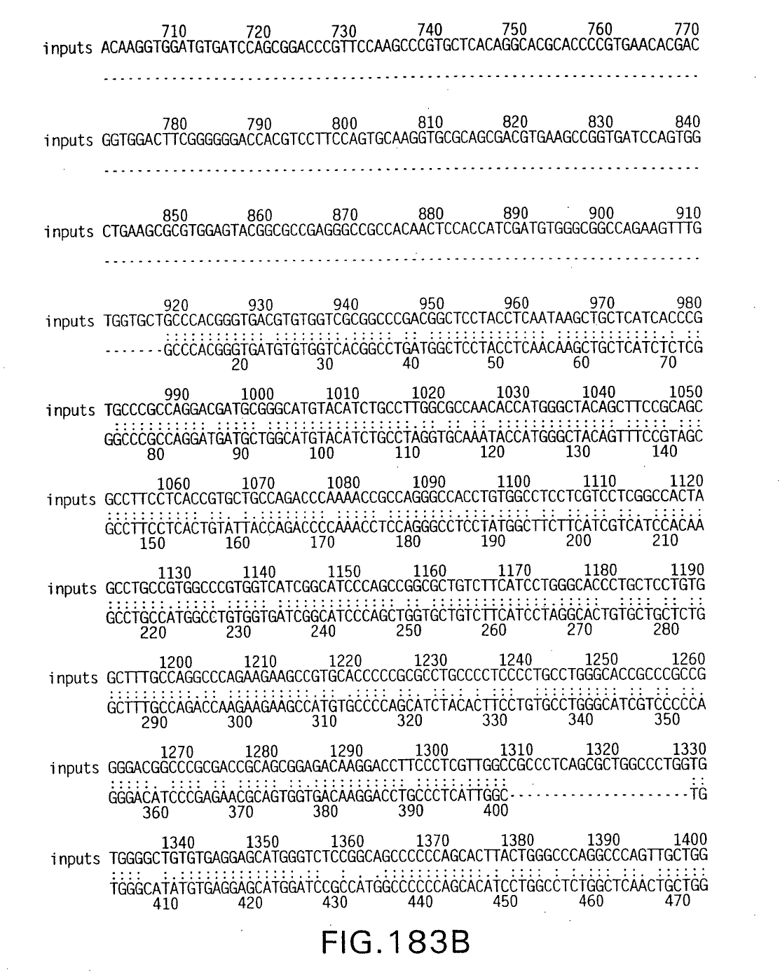

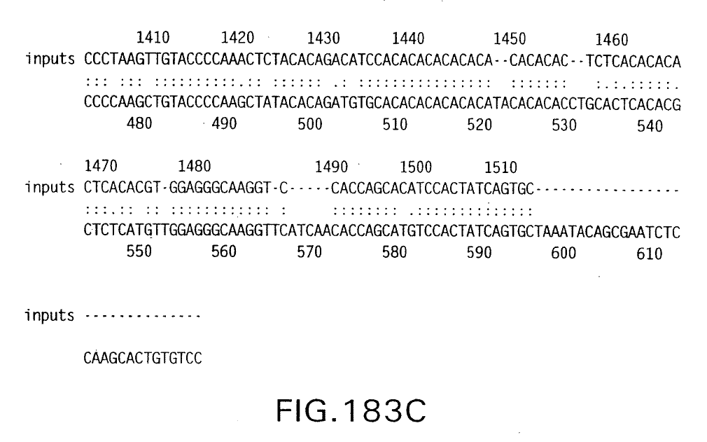

A global alignment between the nucleotide sequence of the open reading frame (ORF) of human MANGO 003 and the nucleotide sequence of the open reading frame of mouse MANGO 003 revealed a 31.1% identity (FIG. 183A-183C). The global alignment was performed using the ALIGN program version 2.0u (Matrix file used: pam 120.mat, gap penalties of −12/−4 with a global alignment score of −1212; Myers and Miller, 1989 CABIOS 4:11-7).

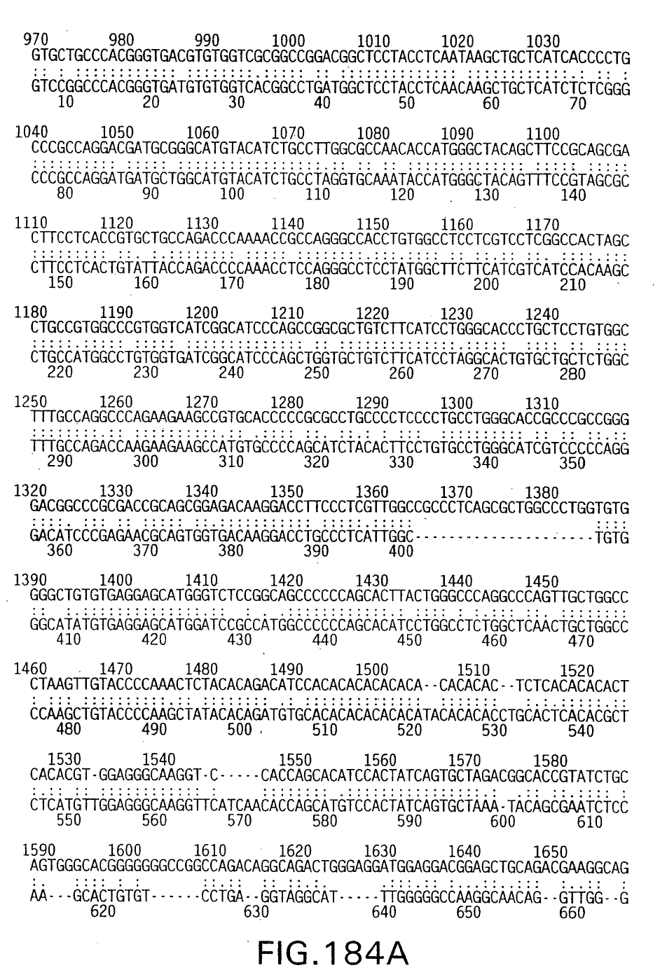

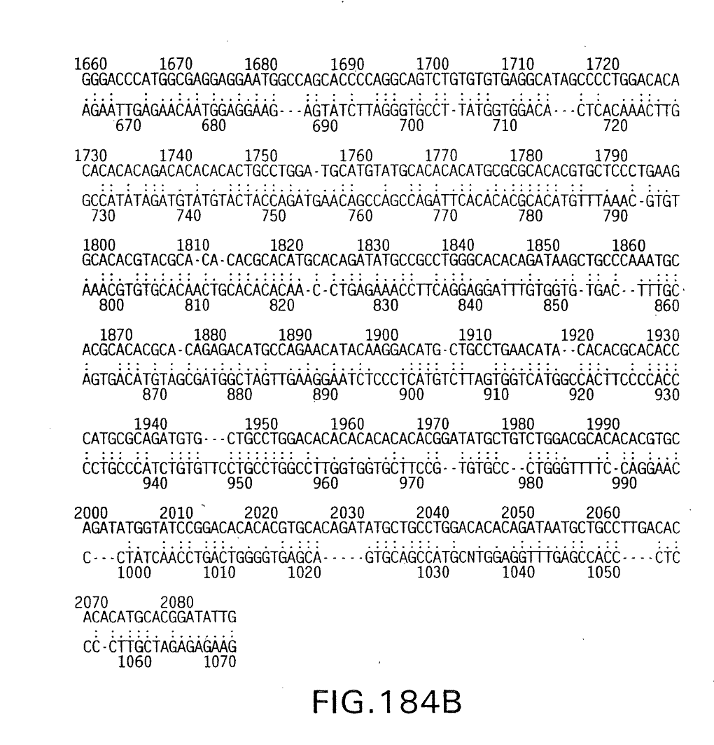

A local alignment between the nucleotide sequence of human MANGO 003 and the nucleotide sequence of mouse MANGO 003 revealed a 62.8% identity over nucleotides 970-2080 of the human MANGO 003 sequence (nucleotides 10-1070 of mouse MANGO 003) (FIG. 184A-184B). The local alignment was performed using the L-ALIGN program version 2.0u4 Jul. 1996 (Matrix file used: pam 120.mat, gap penalties of −12/−4 with a score of 3241; Huang and Miller, 1991, Adv. Appl. Math. 12:373-81).

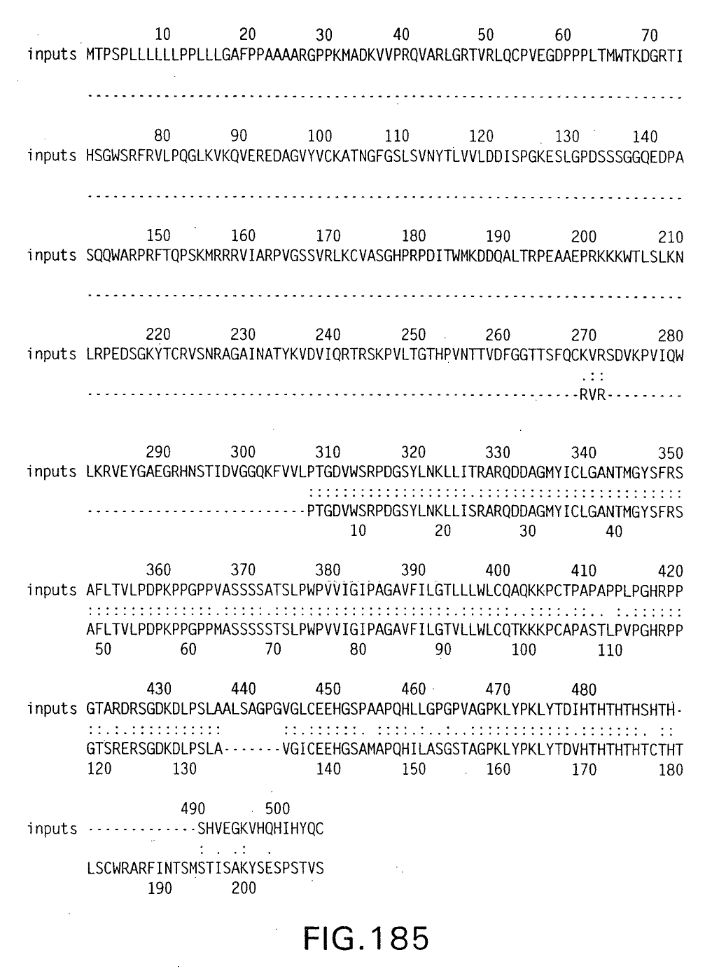

A global alignment between the amino acid sequence of human MANGO 003 and the amino acid sequence of mouse MANGO 003 revealed a 30.1% identity (FIG. 185). The global alignment was performed using the ALIGN program version 2.0u (Matrix file used: pam 120.mat, gap penalties of −12/−4 with a global alignment score of X88; Myers and Miller, 1989, CABIOS 4:11-7).

### Use of MANGO 003 Nucleic Acids, Polypeptides, and Modulators Thereof

MANGO 003 includes three immunoglobulin-like domains. Proteins having such domains play a role in mediating protein-protein and protein-ligand interactions, and thus can influence a wide variety of biological processes, including cell surface recognition; transduction of an extracellular signal (e.g., by interacting with a ligand and/or a cell-surface receptor); and/or modulation of signal transduction pathways.

MANGO 003 further includes a neurotransmitter-gated ion channel domain. Proteins having such domains play a role in modulating signal transmission at chemical synapses by, for example, influencing processes, such as the release of neurotransmitters from a cell (e.g., a neuronal cell); modulating membrane excitability and/or resting potential; and/or modulating ion flux across a membrane of a cell (e.g., a neuronal or a muscle cell). Because MANGO 003 includes a neurotransmitter-gated ion channel domain, MANGO 003 polypeptides, nucleic acids, and modulators thereof can be used to treat neural disorders (e.g., a CNS disorder, including Alzheimer's disease, Pick's disease, Parkinson's and other Lewy diffuse body diseases, multiple sclerosis, amyotrophic lateral sclerosis, progressive supranuclear palsy, epilepsy, and Jakob-Creutzfieldt disease; psychiatric disorders, e.g., depression, schizophrenic disorders, Korsakoffs psychosis, mania, anxiety disorders, or phobic disorders; learning or memory disorders, e.g. amnesia or age-related memory loss; and neurological disorders, e.g., migraine).

MANGO 003 polypeptides, nucleic acids, and modulators thereof can be used to modulate function, survival, morphology, migration, proliferation and/or differentiation of cells in the tissues in which it is expressed (e.g. thyroid, liver, skeletal muscle, kidney, heart, lung, testis and brain). For example, MANGO 003 polypeptides, nucleic acids, and modulators thereof can be used to modulate endocrine, hepatic, skeletal muscular, renal, cardiac, reproductive and/or brain function. Accordingly, these molecules can be used to treat a variety of disease including, but not limited to, endocrine disorders (e.g., hypothyroidism, hyperthyroidism, dwarfism, giantism, acromegaly); hepatic disorders (e.g., hepatitis, liver cirrhosis, hepatoma, liver cysts, and hepatic vein thrombosis); skeletal muscular disorders; renal disorders (e.g., renal cell carcinoma, nephritis, polycystic kidney disease); cardiovascular disorders (e.g., atherosclerosis, ischemia reperfusion injury, cardiac hypertrophy, hypertension, coronary artery disease, myocardial infarction, arrhythmia, cardiomyopathies, and congestive heart failure); and/or reproductive disorders (e.g., sterility).

MANGO 003 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, MANGO 003 polypeptides, nucleic acids, or modulators thereof, can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g. Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofuctokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, MANGO 003 polypeptides, nucleic acids, or modulators thereof, can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g. renal cell carcinoma and nephroblastoma).

Further, in light of MANGO 003's pattern of expression in mice, MANGO 003 expression can be utilized as a marker for specific tissues (e.g., liver, skeletal muscle, kidney) and/or cells (e.g., hepatic, skeletal muscle, renal) in which MANGO 003 is expressed. MANGO 003 nucleic acids can also be utilized for chromosomal mapping.

### MANGO 347

A cDNA encoding human MANGO 347 was identified by analyzing the sequences of clones present in a human brain cDNA library.

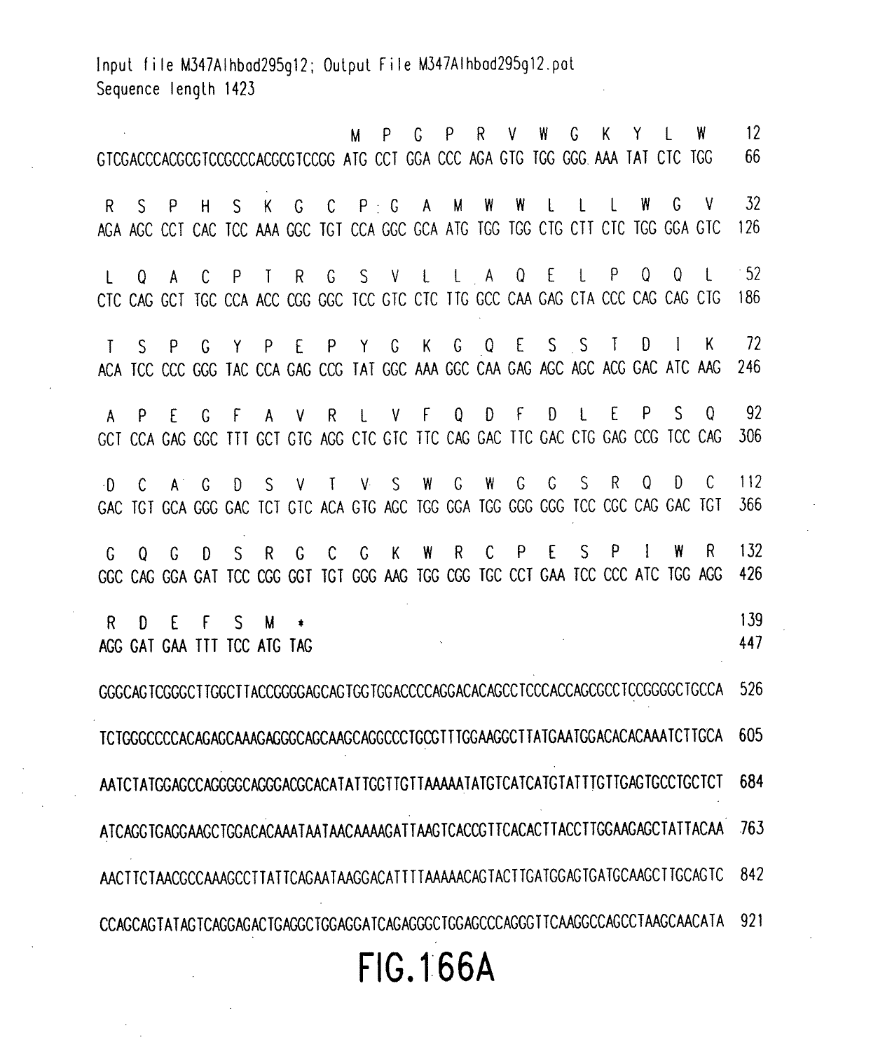

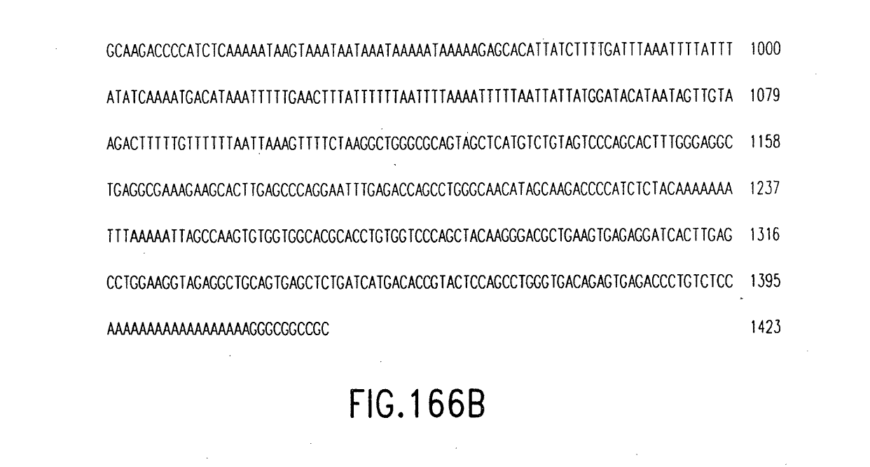

This analysis led to the identification of a clone, jlhbad295g12, encoding full-length human MANGO 347. The cDNA of this clone is 1423 nucleotides long (FIG. 166A-166B; SEQ ID NO: 111). The 414 nucleotide open reading frame of this cDNA (nucleotides 31 to 444 of SEQ ID NO: 111) encodes a 138 amino acid protein (SEQ ID NO: 112).

The signal peptide prediction program SIGNALP (Nielsen et al., 1997, Protein Engineering 10:1-6) predicted that human MANGO 347 includes a 35 amino acid signal peptide at amino acid 1 to about amino acid 35 preceding the mature human MANGO 347 protein which corresponds to about amino acid 36 to amino acid 138 of SEQ ID NO:112.

Human MANGO 347 that has not been post-translationally modified is predicted to have a molecular weight of 15.4 kDa prior to cleavage of its signal peptide and a molecular weight of 11.3 kDa subsequent to cleavage of its signal peptide.

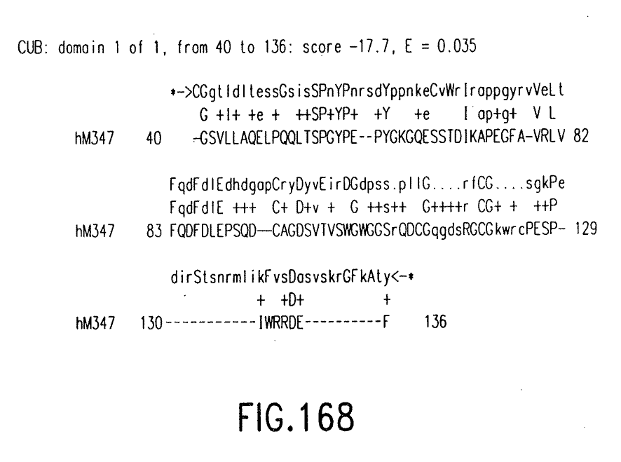

MANGO 347 family members can include a CUB domain sequence. As used herein, the term “CUB domain” includes an amino acid sequence having at least about 80-150, preferably 90-130, more preferably 96-120, and most preferably about 110 amino acids in length. Preferably, a CUB domain further includes at least one, preferably two, three, and most preferably four conserved cysteine residues. Preferably, the conserved cysteine residues form at least one, and preferably two disulfide bridges (e.g., Cys1-Cys2, and Cys3-Cys4) resulting in a β-barrel configuration. The CUB domain of MANGO 347 extends from about amino acid 40 to amino acid 136 of SEQ ID NO: 112. FIG. 168 depicts an alignment of the consensus hidden Markov model CUB domain with this domain in human MANGO 347 at amino acids 40 to 136 of SEQ ID NO:112.

In one embodiment, a MANGO 354 family member includes at least one immunoglobulin domain and a transmembrane domain. In another embodiment, a MANGO 354 family member includes at least one immunoglobulin domain, a transmembrane domain and a signal peptide.

Human MANGO 347 includes a CUB domain at amino acids 40-136 of SEQ ID NO:112. An alignment of the CUB domain of human MANGO 347 with a consensus hidden Markov model CUB domain amino acid sequence derived from a hidden Markov model is shown in FIG. 168.

Casein kinase II phosphorylation sites are present at amino acids 67-70, and 108-111. N-myristylation sites are present at amino acids 19-24, 31-36, 64-69, and 113-118.

Clone jlhbad295 g12, which encodes human MANGO 347, was deposited as a composite deposit having a designation EpM347 with the American Type Culture Collection (ATCC® 10801 University Boulevard, Manassas, Va. 20110-2209) on Jun. 18, 1999 and assigned Accession Number PTA-250. A description of the deposit conditions used in set forth below. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

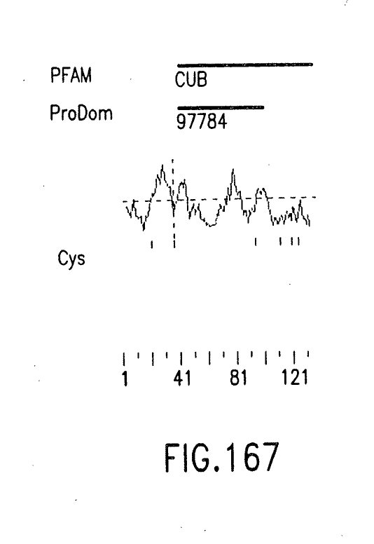

FIG. 167 depicts a hydropathy plot of human MANGO 347. The hydropathy plot indicates that human MANGO 347 has a signal peptide at its amino terminus, suggesting that human MANGO 347 is a secreted protein.

### Use of MANGO 347 Nucleic Acids, Polypeptides, and Modulators Thereof

MANGO 347 includes a CUB domain. Proteins having such a domain play a role in mediating cell interactions during development, and thus can influence a wide variety of developmental processes, including morphogenesis, cellular migration, adhesion, proliferation, differentiation, and/or survival. MANGO 347 polypeptides are expressed in neural (e.g., brain cells). Because MANGO 347 includes a CUB domain and is expressed in neural cells, MANGO 347 polypeptides, nucleic acids, and modulators thereof can be used to treat disorders involving, e.g., cellular migration, proliferation, and differentiation of a cell, e.g., a neural cell (e.g., a CNS disorder, including Alzheimer's disease, Pick's disease, Parkinson's and other Lewy diffuse body diseases, multiple sclerosis, amyotrophic lateral sclerosis, progressive supranuclear palsy, epilepsy, and Jakob-Creutzfieldt disease; psychiatric disorders, e.g., depression, schizophrenic disorders, Korsakoff's psychosis, mania, anxiety disorders, or phobic disorders; learning or memory disorders, e.g., amnesia or age-related memory loss; and neurological disorders, e.g., migraine).

Further, in light of MANGO 347's presence in a human brain cDNA library, MANGO 347 expression can be utilized as a marker for specific tissues (e.g., brain) and/or cells (e.g., brain) in which MANGO 347 is expressed. MANGO 347 nucleic acids can also be utilized for chromosomal mapping.

### TANGO 272

A cDNA encoding human TANGO 272 was identified by analyzing the sequences of clones present in a human microvascular endothelial cell library (HMVEC) cDNA library.

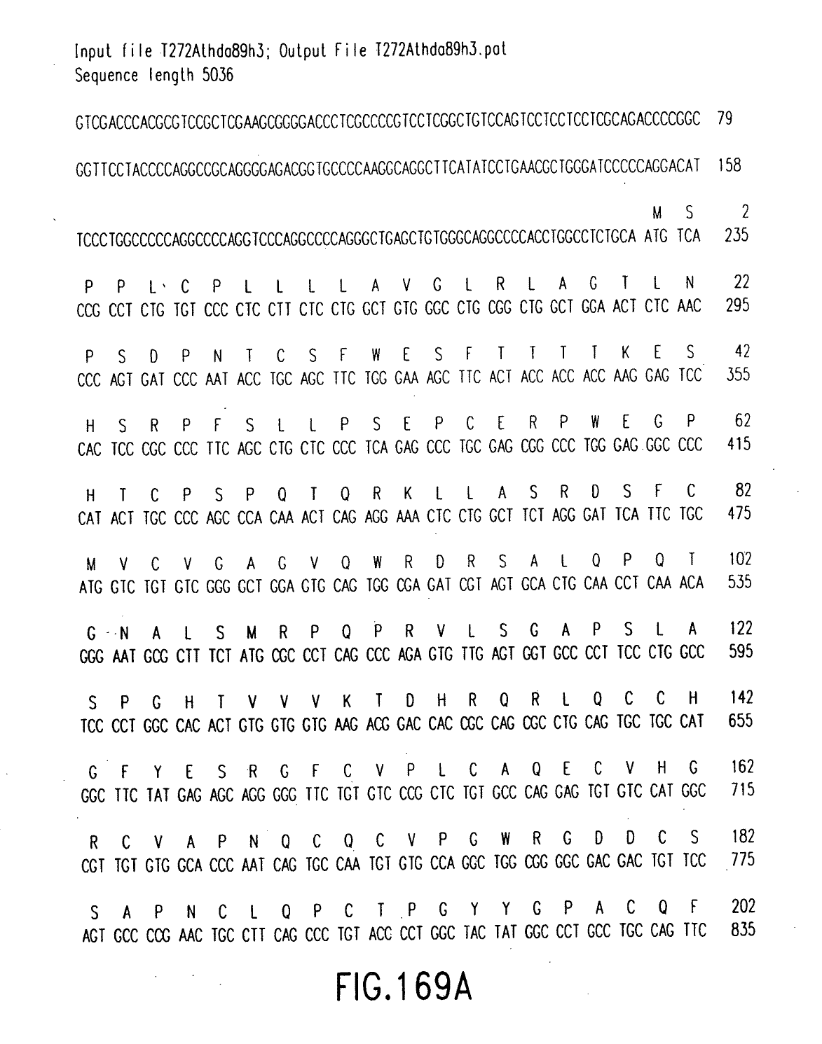

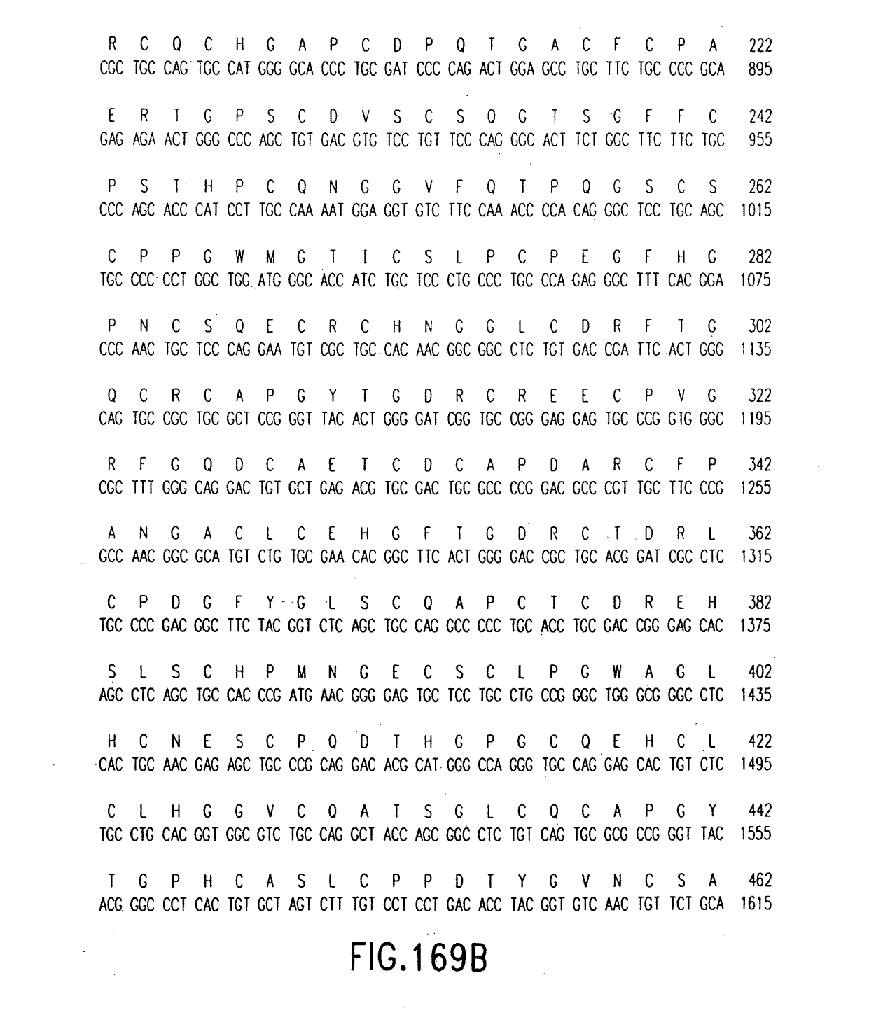

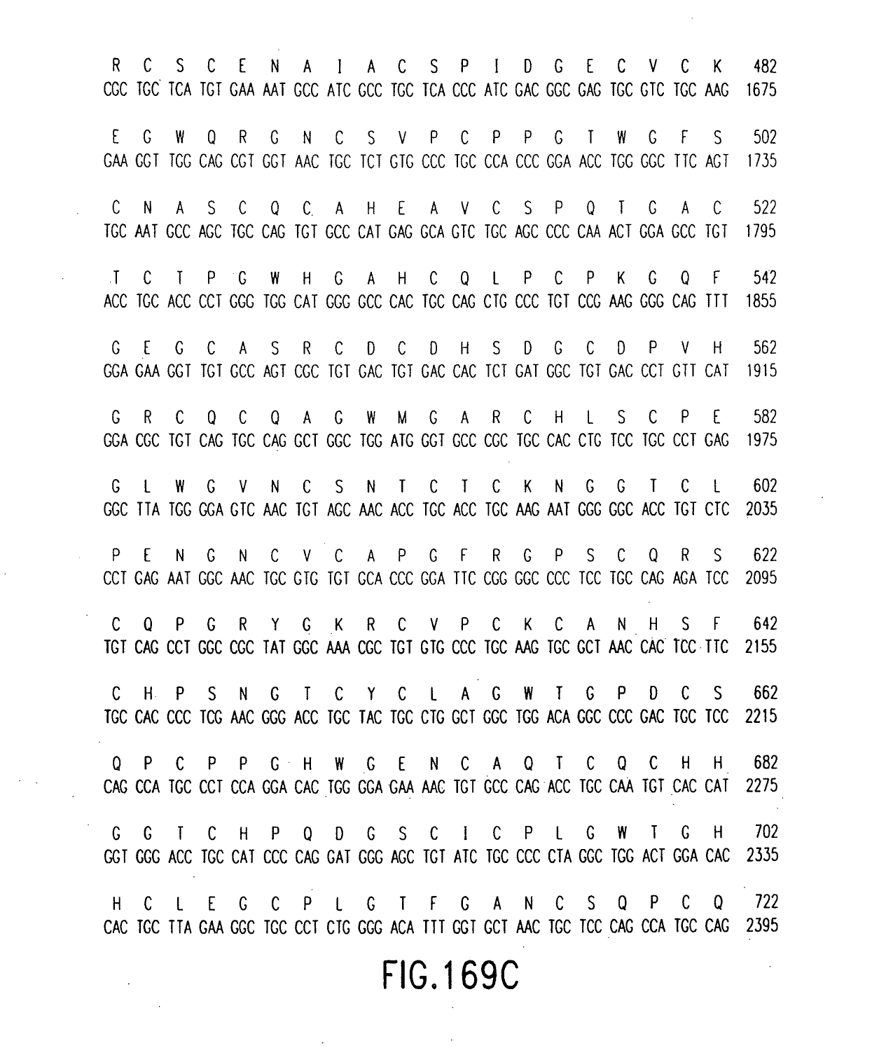

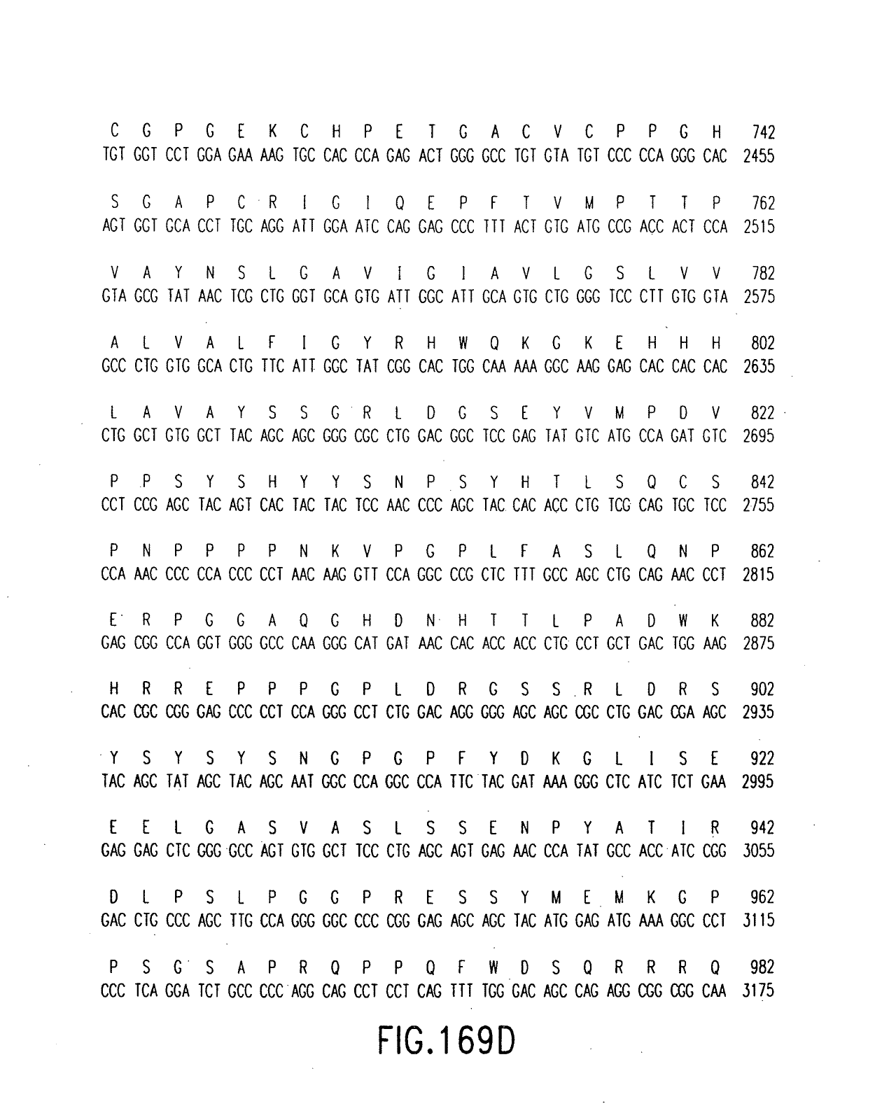

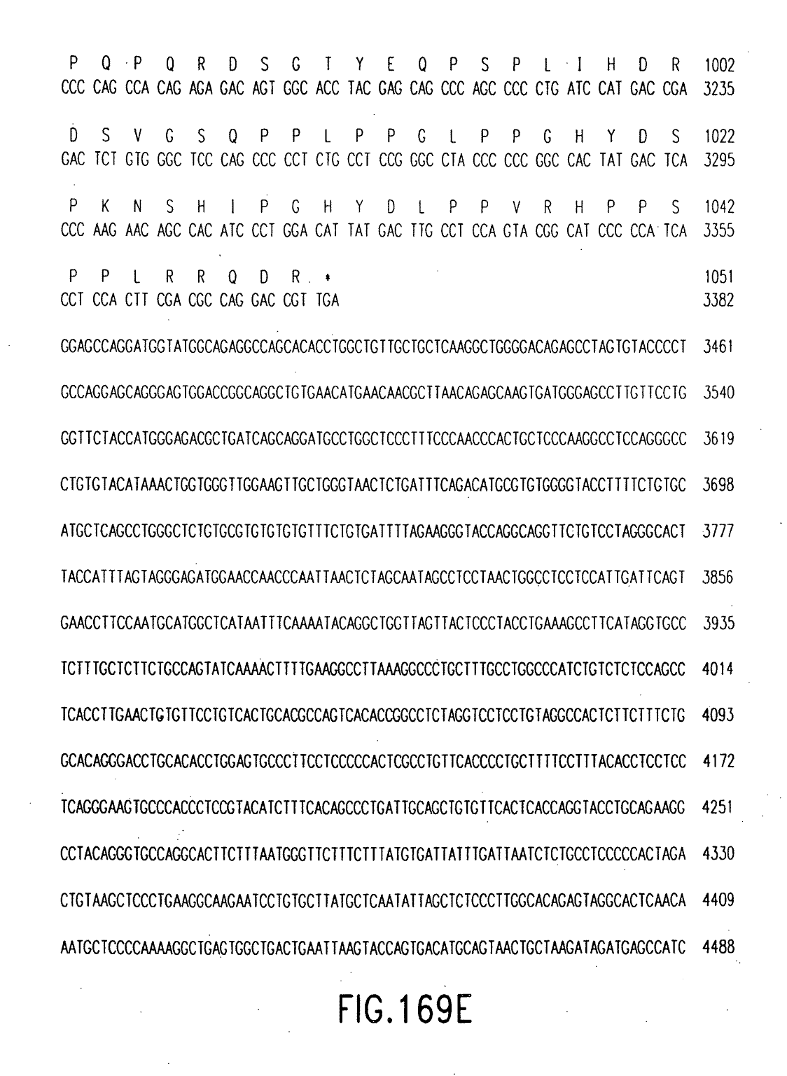

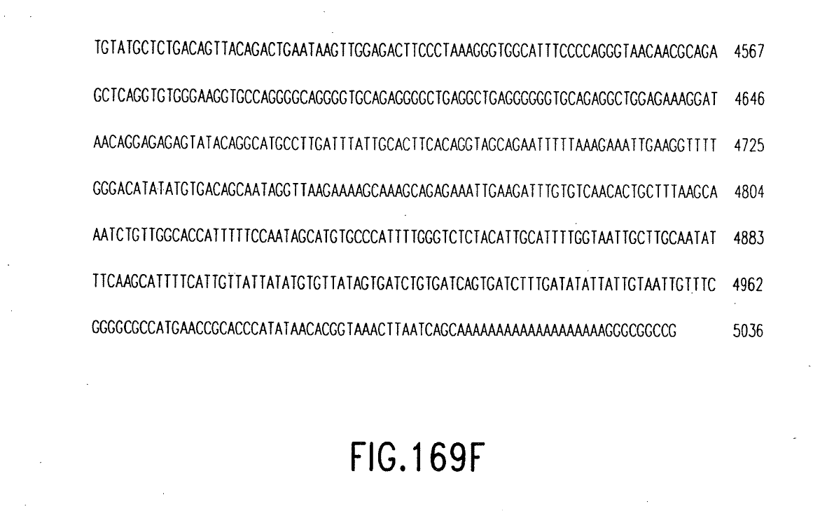

This analysis led to the identification of a clone, jthda089h03, encoding full-length human TANGO 272. The cDNA of this clone is 5036 nucleotides long (FIG. 169A-169F; SEQ ID NO: 113). The 3149 nucleotide open reading frame of this cDNA (nucleotides 230-3379 of SEQ ID NO:113) encodes a 1050 amino acid protein (SEQ ID NO:114).

Northern blot analysis using the human clone jthda089h03 revealed strong expression of the human TANGO 272 gene in the heart. Moderate expression was detected in the placenta, lung, and liver, and lower levels of expression were detected in the brain, skeletal muscle, kidney, and pancreas.

The signal peptide prediction program SIGNALP (Nielsen et al., 1997, Protein Engineering 10:1-6) predicted that human TANGO 272 includes an 20 amino acid signal peptide at amino acid 1 to about amino acid 20 preceding the mature human TANGO 272 protein which corresponds to about amino acid 21 to amino acid 1050 of SEQ ID NO:114.

Human TANGO 272 that has not been post-translationally modified is predicted to have a molecular weight of 112 kDa prior to cleavage of its signal peptide and a molecular weight of 110 kDa subsequent to cleavage of its signal peptide.

Human TANGO 272 is a transmembrane protein having an extracellular domain which extends from about amino acid 21 to about amino acid 767, a transmembrane domain which extends from about amino acid 768 to about amino acid 791, and a cytoplasmic domain which extends from about amino acid 792 to amino acid 1050 of SEQ ID NO:114.

Alternatively, in another embodiment, a human TANGO 272 protein contains an extracellular domain which extends from about amino acid 792 to amino acid 1050, a transmembrane domain which extends from about amino acid 768 to about amino acid 791, and a cytoplasmic domain which extends from about amino acid 21 to about amino acid 767 of SEQ ID NO:114.

TANGO 272 family members can include at least one, two, three, four, five, six, seven, eight, nine, ten, eleven, twelve, preferably thirteen, and more preferably fourteen EGF-like domains. Preferably, the EGF-like domains are found in the extracellular domain of a TANGO 272 protein. As used herein, an “EGF-like domain” refers to an amino acid sequence of about 25 to 50, preferably about 30 to 45, and more preferably 30 to 40 amino acid residues in length. An EGF domain further contains at least about 2 to 10, preferably, 3 to 9, 4 to 8, or 6 to 7 conserved cysteine residues. A consensus hidden Markov model EGF-like domain sequence includes six cysteines, all of which are thought to be involved in disulfide bonds having the following amino acid sequence: Cys-Xaa(5, 7)-Cys-Xaa(4, 5, 12)-Cys-Xaa(1, 5, 6)-Cys-Xaa(1)-Cys-Xaa(1)-Cys-Xaa(8)-Cys, where Xaa is any amino acid. The region between the fifth and the sixth cysteine typically contains two conserved glycines of which at least one is present in most EGF-like domains.

In one embodiment, TANGO 272 includes at least one EGF-like domain having the sequences selected from the group consisting of: amino acids 151-181; amino acids 200-229; amino acids 242-272; amino acids 285-315; amino acids 328-358; amino acids 378-404; amino acids 417-447; amino acids 460-490; amino acids 503-533; amino acids 546-576; amino acids 589-619; amino acids 632-661; amino acids 674-704; and amino acids 717-747 of SEQ ID NO: 114.

In another embodiment, TANGO 272 includes at least one EGF-like domain having the sequences selected from the group consisting of: 37-67; amino acids 80-110; amino acids 123-153; and amino acids 166-196.

In yet another embodiment, TANGO 272 includes at least one EGF-like domain having the sequences selected from the group consisting of: amino acids 18-48; amino acids 61-91; amino acids 105-137; amino acids 150-180; amino acids 193-223; amino acids 236-266; amino acids 279-309; amino acids 322-352; amino acids 365-394; amino acids 407-437; and amino acids 450-480.

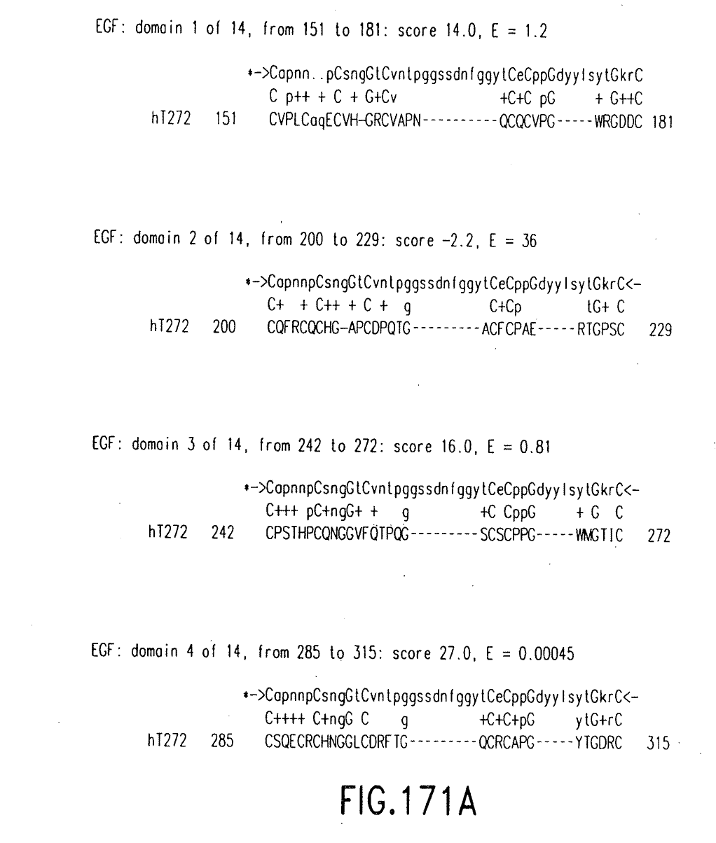

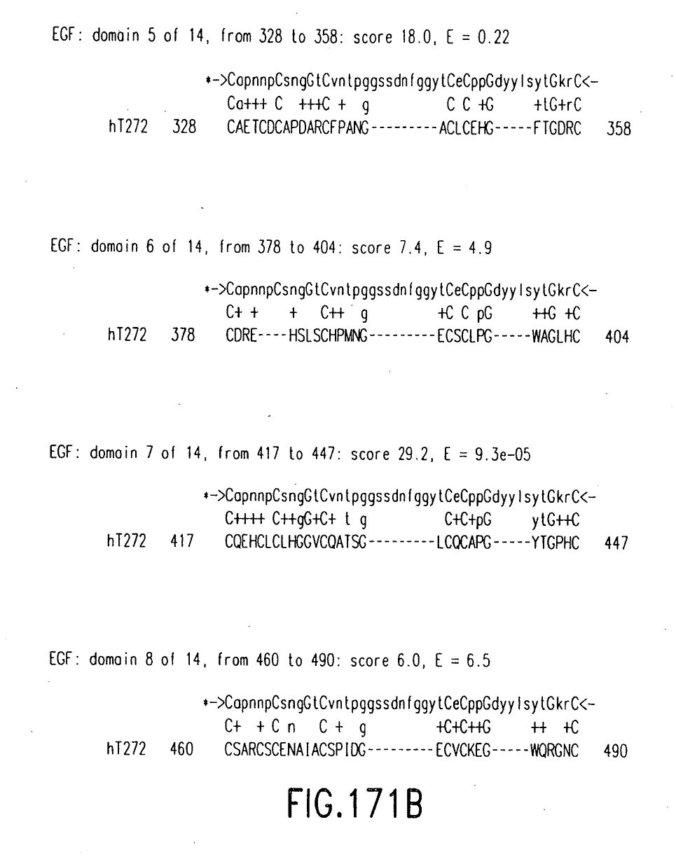

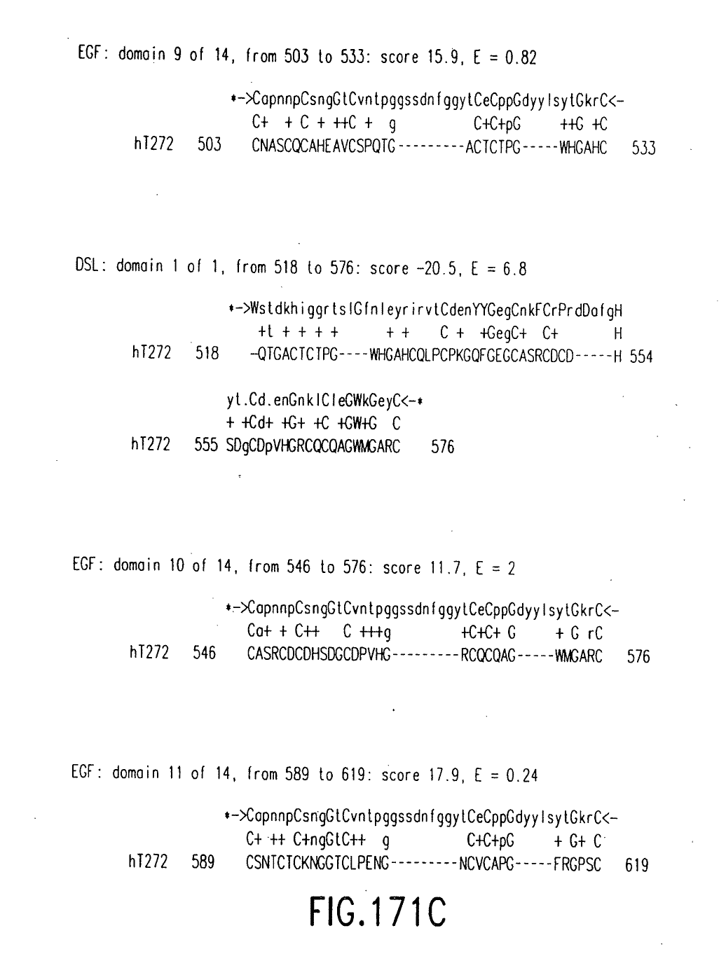

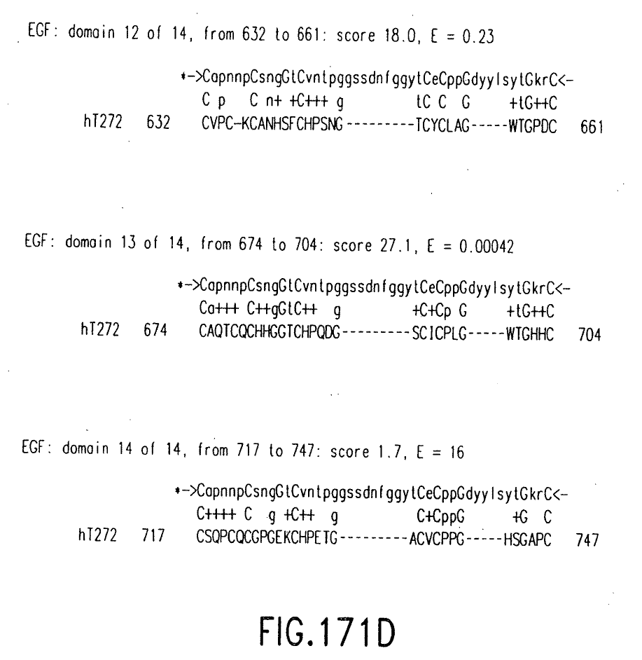

An alignment of the consensus hidden Markov model EGF-like domains with the EGF-like domains of human TANGO 272 is shown in FIG. 171A-171D. The more conserved residues in the consensus sequence are indicated by uppercase letters and the less conserved residues in the consensus sequence are indicated by lowercase letters. By alignment of the amino acid sequence of the consensus hidden Markov model EGF-like domain with the amino acid sequence of the EGF-like domains of TANGO 272, conserved cysteine residues can be found. For example, conserved cysteine residues can be found at amino acid 151, 159, 164, 167, 200, 206, 211, 218, 220, 229, 242, 249, 263, 264, 272, 285, 291, 297, 304, 306, 315, 328, 334, 340, 347, 349, 358, 378, 386, 393, 395, 404, 417, 423, 429, 436, 438, 447, 460, 466, 472, 479, 481, 490, 503, 509, 515, 522, 524, 533, 546, 552, 558, 565, 567, 576, 589, 595, 601, 608, 610, 619, 632, 637, 643, 650, 652, 661, 674, 680, 686, 693, 695, 717, 723, 729, 736, 738 and 747 of SEQ ID NO:114.

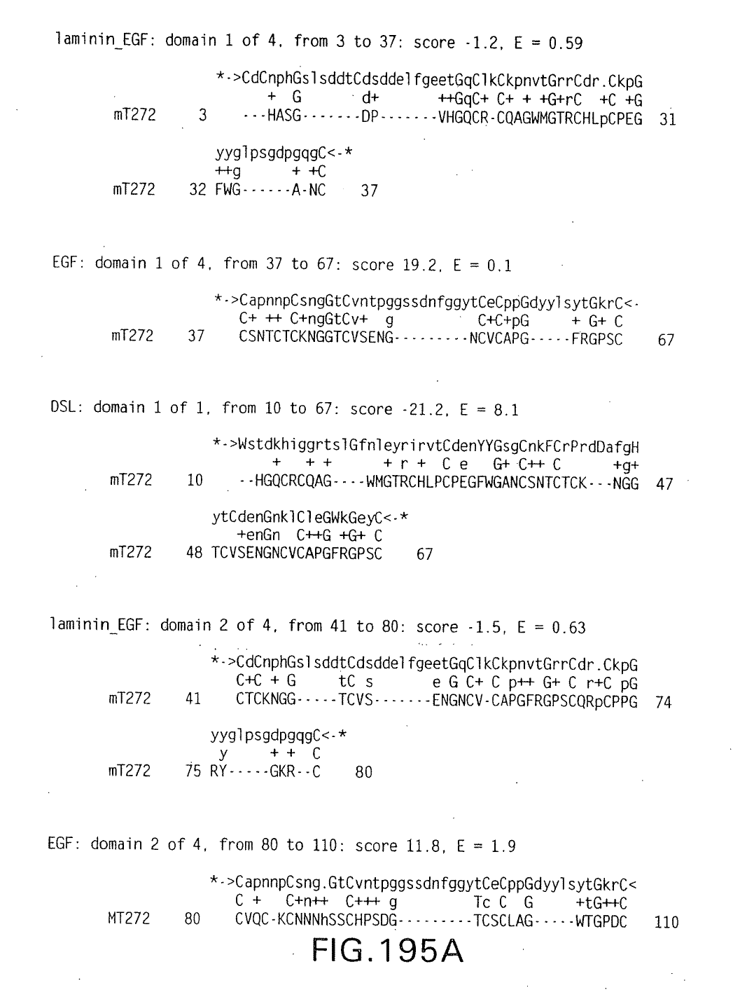

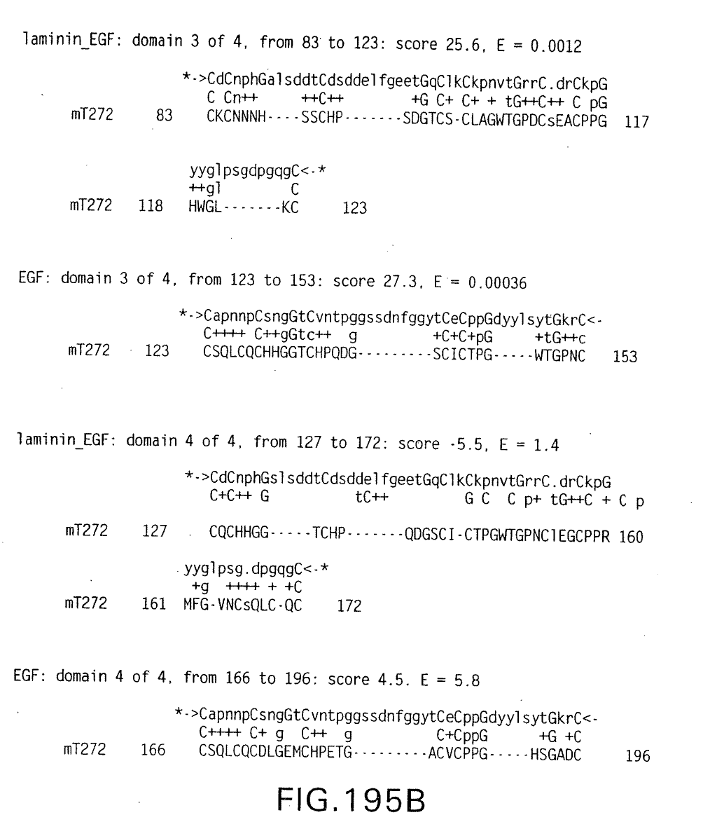

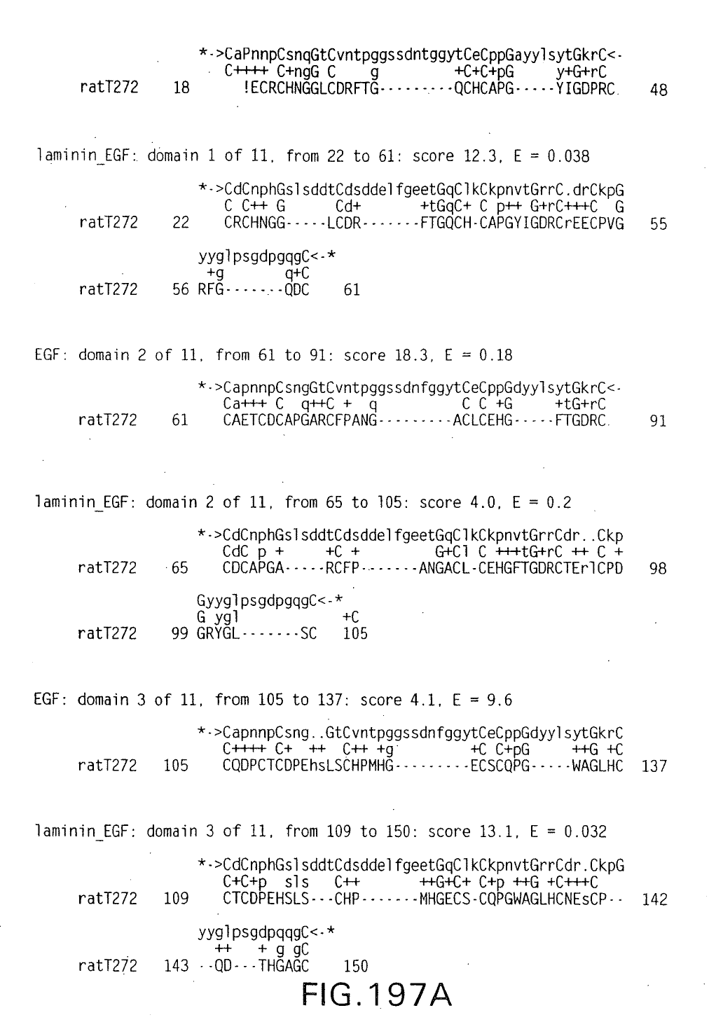

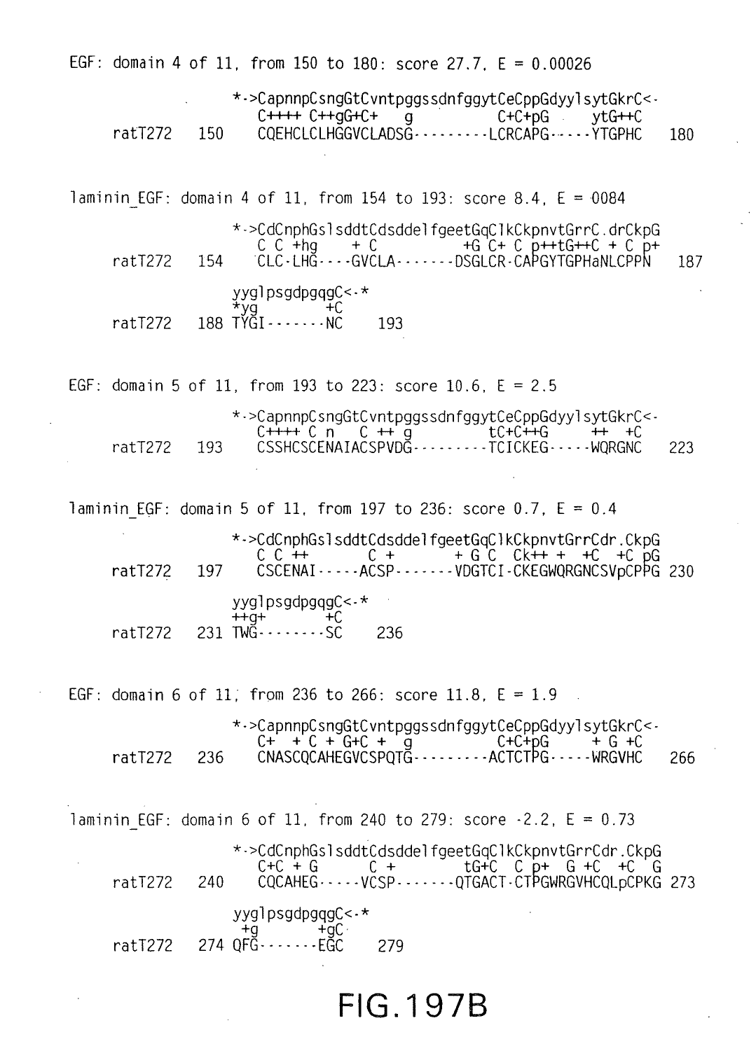

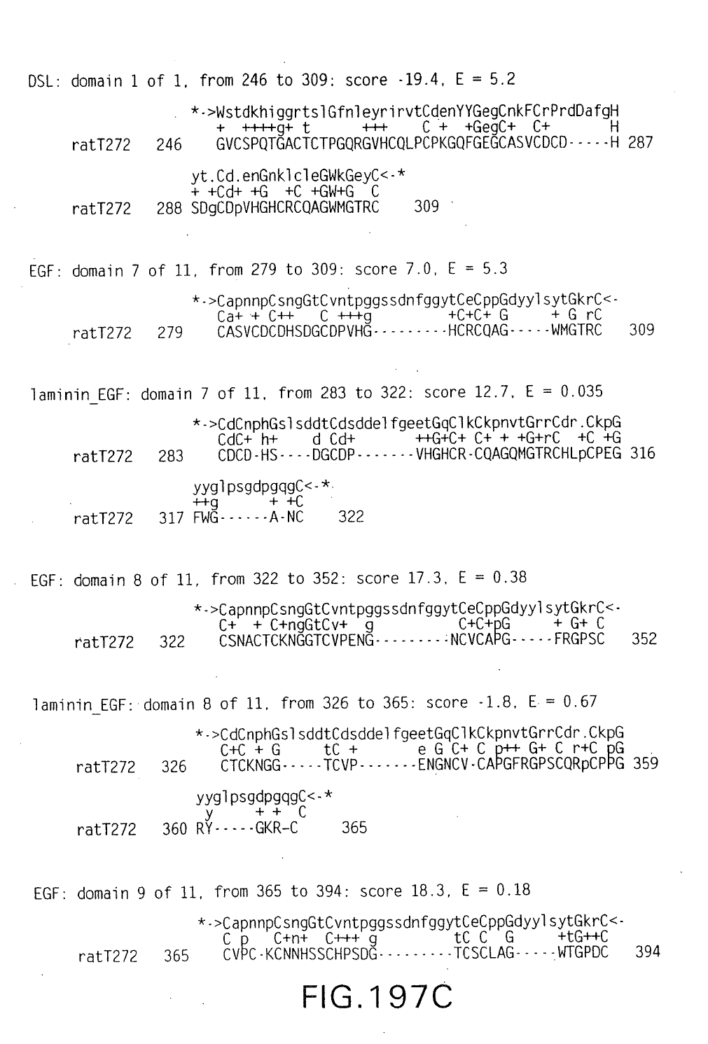

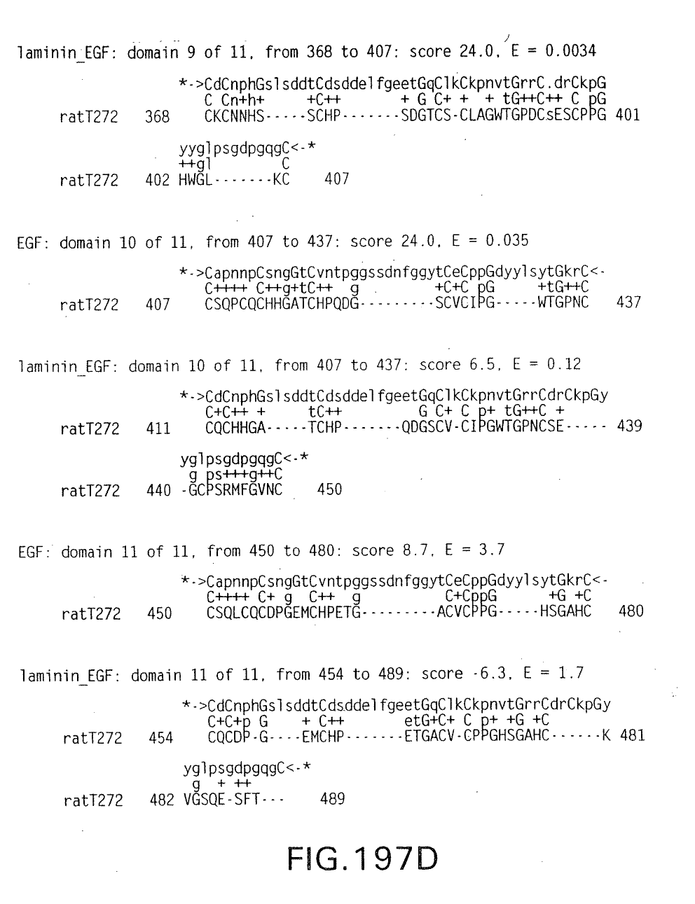

TANGO 272 family members can include at least one delta serrate ligand domain. As used herein, a “delta serrate ligand domain” (also referred to herein as a “DSL domain”) refers to an amino acid sequence of about 30-70, more preferably 45-60, and most preferably 58 amino acids in length typically found in transmembrane signaling molecules that regulate differentiation in metazoans (Lissemore et al., 1999, Mol. Phylogenet. Evol. 11(2):308-19). In one embodiment, human TANGO 272 includes a delta serrate ligand domain from about amino acids 518 to 576; and about amino acids 246 to 309 of SEQ ID NO: 114. FIG. 171C depicts an alignment of the consensus hidden Markov model delta serrate ligand domain with this domain in human TANGO 272 at amino acids 518 to 576 of SEQ ID NO:114. FIG. 195A-195B depicts an alignment of the consensus hidden Markov model delta serrate ligand domain with this domain in mouse TANGO 272 at amino acids 10 to 67 of SEQ ID NO: 114. FIG. 197C depicts an alignment of the consensus hidden Markov model delta serrate ligand domain with this domain in rat TANGO 272 at amino acids 246 to 309 of SEQ ID NO:114.

TANGO 272 family members can include at least one RGD cell attachment site. As used herein, the term “RGD cell attachment site” refers to a cell adhesion sequence consisting of amino acids Arg-Gly-Asp typically found in extracellular matrix proteins such as collagens, laminin and fibronectin, among others (reviewed in Ruoslahti, 1996, Annu. Rev. Cell Dev. Biol. 12:697-715). Preferably, the RGD cell attachment site is located in the extracellular domain of a TANGO 272 protein and interacts (e.g., binds to) a cell surface receptor, such as an integrin receptor. As used herein, the term “integrin” refers to a family of receptors comprising a/p heterodimers that mediate cell attachment to extracellular matrices and cell-cell adhesion events. The α subunits vary in size between 120 and 180 kDa and are each noncovalently associated with a β subunit (90-110 kDa) (reviewed by Hynes, 1992, Cell 69:11-25). Most integrins are expressed in a wide variety of cells, and most cells express several integrins. There are at least 8 known α subunits and 14 known p subunits. The majority of the integrin ligands are extracellular matrix proteins involved in substratum cell adhesion such as collagens, laminin, fibronectin among others. The RGD cell attachment site is located at about amino acid residues 177-179.

In one embodiment, a TANGO 272 family member includes fourteen EGF-like domains and a delta serrate ligand domain. In another embodiment, a TANGO 272 family member includes fourteen EGF-like domains, a delta serrate ligand domain and an RGD cell attachment site. In yet another embodiment, a TANGO 272 family member includes fourteen EGF-like domains, a delta serrate ligand domain, an RGD cell attachment site, and a transmembrane domain. In another embodiment, a TANGO 272 family member includes fourteen EGF-like domains, a delta serrate ligand domain, an RGD cell attachment site, a transmembrane domain, and an extracellular N-terminal domain. In another embodiment, a TANGO 272 family member includes fourteen EGF-like domains, a delta serrate ligand domain, an RGD cell attachment site, a transmembrane domain, an extracellular N-terminal domain and a C-terminal cytoplasmic domain. In another embodiment, a TANGO 272 family member includes fourteen EGF-like domains, a delta serrate ligand domain, an RGD cell attachment site, a transmembrane domain, an extracellular N-terminal domain, a C-terminal cytoplasmic domain, and a signal peptide.

Human TANGO 272 includes fourteen EGF-like domains at amino acids 151-181; amino acids 200-229; amino acids 242-272; amino acids 285-315; amino acids 328-358; amino acids 378-404; amino acids 417-447; amino acids 460-490; amino acids 503-533; amino acids 546-576; amino acids 589-619; amino acids 632-661; amino acids 674-704; and amino acids 717-747 of SEQ ID NO:114. FIG. 171A-171D depicts alignments of each of the EGF-like domains of TANGO 272 with consensus hidden Markov model EGF-like domains. Human TANGO 272 further includes a delta serrate ligand domain from amino acids 518 to 576. An alignment of the delta serrate ligand domain of human TANGO 272 with a consensus hidden Markov model of this domain is depicted (FIG. 171C).

An RGD cell attachment site is present at amino acids 177-179. N-glycosylation sites are present at amino acids 284-287, 405-408, 459-462, 489-492, 504-507, 588-591, 639-642, 647-650, 716-719, and 873-876. An amidation site is present at amino acids 628-631. Protein kinase C phosphorylation sites are present at amino acids 3840, 70-72, 107-109, 359-361, 461-463, 594-596, 809-811, 896-898, 940-942, 977-979, and 1022-1024. Casein kinase II phosphorylation sites are present at amino acids 30-33, 38-41, 473-476, 548-551, 579-582, 657-660, 897-900, 921-924, 940-943, and 955-958. A tyrosine kinase phosphorylation site is present at amino acids 361-368. N-myristylation sites are present at amino acids 14-19, 103-108, 269-274, 302-307, 325-330, 345-350, 401-406, 427-432, 434-439, 457-462, 520-525, 586-591, 606-611, 648-653, 707-712, 714-719, 769-774, 866-871, 926-931, and 1014-1019.

Clone jthda089h03, which encodes human TANGO 272, was deposited as a composite deposit having a designation EpT272 with the American Type Culture Collection (ATCC® 10801 University Boulevard, Manassas, Va. 20110-2236) Jun. 18, 1999 and assigned Accession Number PTA-250. A description of the deposit conditions used is set forth in the section entitled “Deposit of Clones” below. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

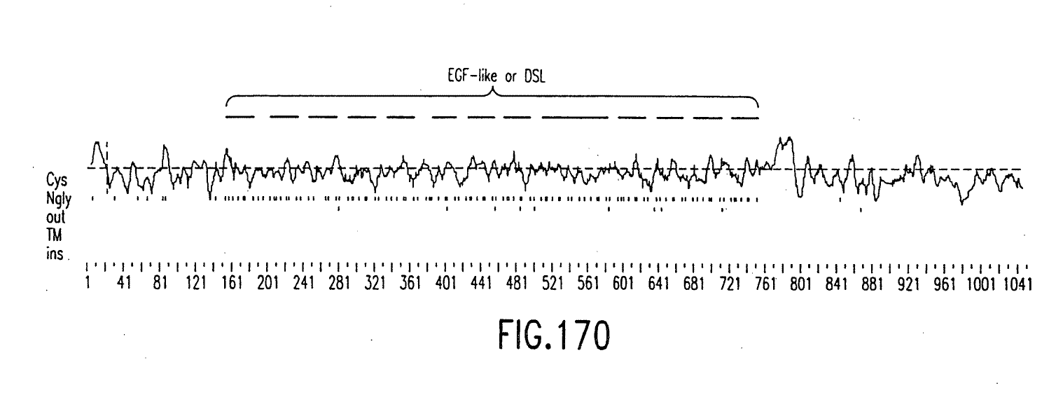

FIG. 170 depicts a hydropathy plot of human TANGO 272. The hydropathy plot indicates the presence of a hydrophobic domain within human TANGO 272, suggesting that human TANGO 272 is a transmembrane protein.

A cDNA encoding mouse TANGO 272 was identified by analyzing the sequences of clones present in a mouse testis cDNA library.

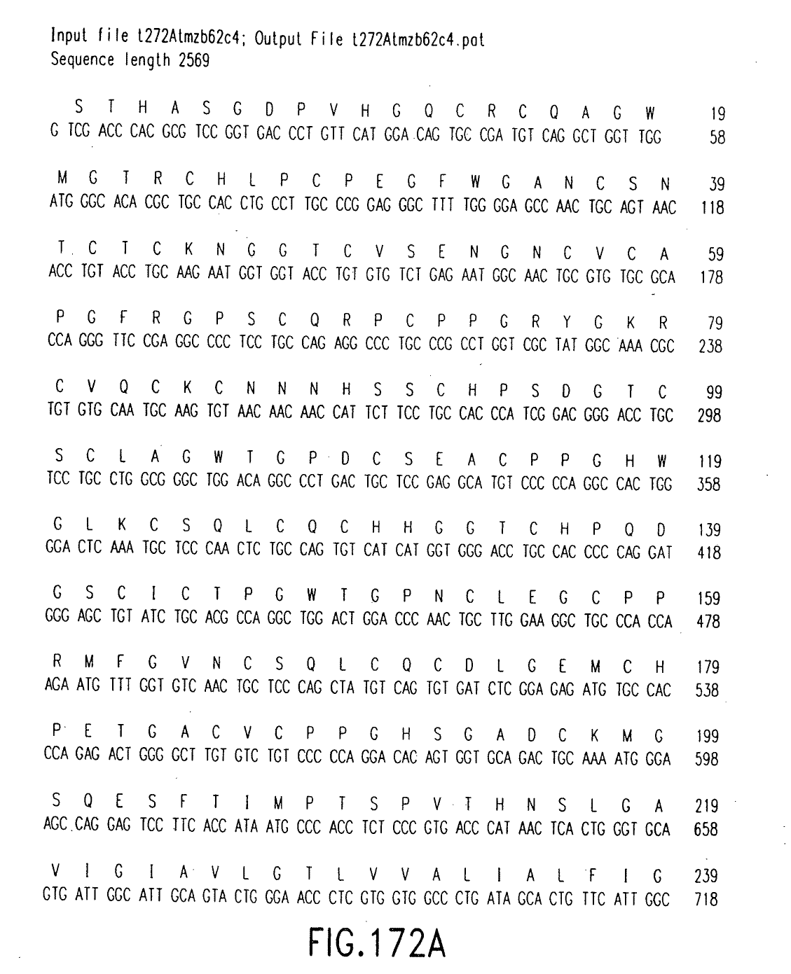

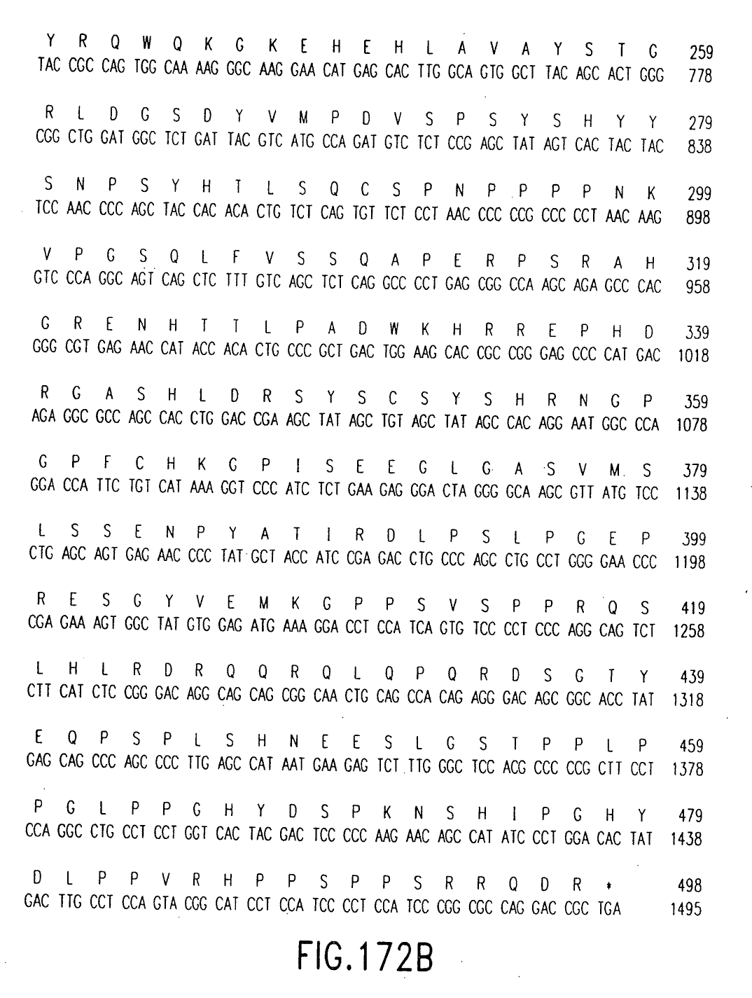

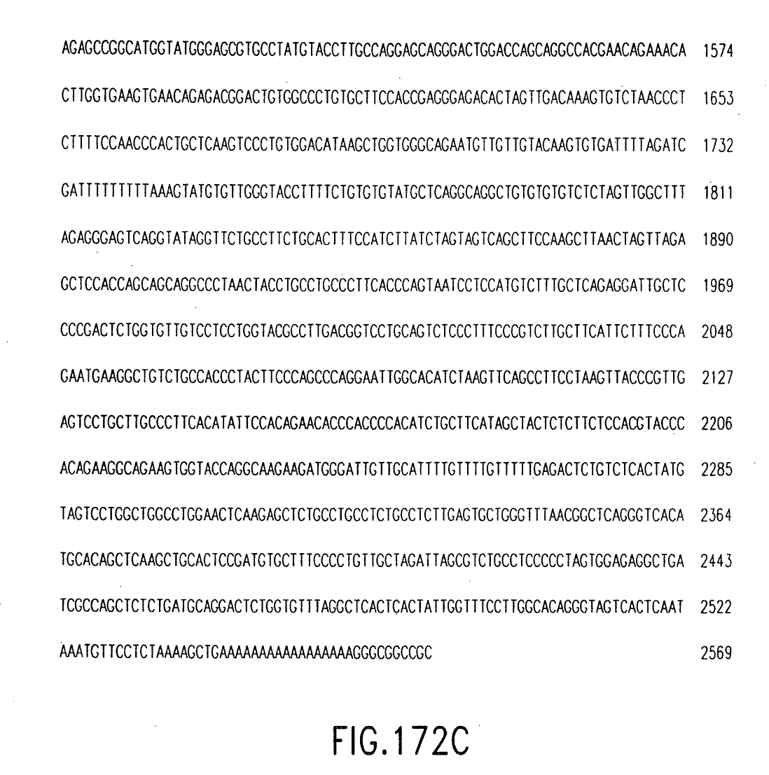

This analysis led to the identification of a clone, jtmzb062c04, encoding partial mouse TANGO 272. The cDNA of this clone is 2569 nucleotides long (FIG. 172A-172C; SEQ ID NO:115). The 1492 nucleotide open reading frame of this cDNA (nucleotides 1-1492 of SEQ ID NO:115) encodes a 497 amino acid protein (SEQ ID NO:116).

Mouse TANGO 272 that has not been post-translationally modified is predicted to have a molecular weight of 53.5 kDa.

Mouse TANGO 272 is a transmembrane protein having an extracellular domain which extends from about amino acid 1 to about amino acid 216, a transmembrane domain which extends from about amino acid 217 to about amino acid 240, and a cytoplasmic domain which extends from about amino acid 241 to amino acid 497 of SEQ ID NO:116.

Alternatively, in another embodiment, a mouse TANGO 272 protein contains an extracellular domain which extends from about amino acid 241 to amino acid 497, a transmembrane domain which extends from about amino acid 217 to about amino acid 240, and a cytoplasmic domain which extends from about amino acid 1 to about amino acid 216 of SEQ ID NO: 116.

Mouse TANGO 272 includes four EGF-like domains at about amino acids 37-67; amino acids 80-110; amino acids 123-153; and amino acids 166-196. Mouse TANGO 272 further includes four laminin-EGF-like domains at about amino acids 3-37; amino acids 41-80; amino acids 83-123; and amino acids 127-172 of SEQ ID NO:116. FIG. 195A-195B depicts alignments of each of the EGF-like- and laminin-EGF-like domains of TANGO 272 with consensus hidden Markov model EGF-like domains.

Mouse TANGO 272 further includes a delta serrate ligand domain from amino acids 10 to 67 of SEQ ID NO:116. An alignment of the delta serrate ligand domain of mouse TANGO 272 with a consensus hidden Markov model of this domain is also depicted in FIG. 195A.

Based on the Prosite analysis, EGF-like domain cysteine pattern signature are present at amino acids 13-24, 56-67, 99-110, 142-153, and 185-196.

N-glycosylation sites are present at amino acids 36-39, 88-91, 165-168, and 323-326. An amidation site is present at amino acids 76-79. Protein kinase C phosphorylation sites are present at amino acids 42-44, 258-260, 354-356, 388-390, 469-471, and 492-494. Casein kinase II phosphorylation sites are present at amino acids 106-109, 192-195, 343-346, 388-391, and 446-449. N-myristylation sites are present at amino acids 11-16, 34-39, 47-52, 54-59, 97-102, 120-125, 140-145, 163-168, 199-204, 218-223, 372-377, and 461-466.

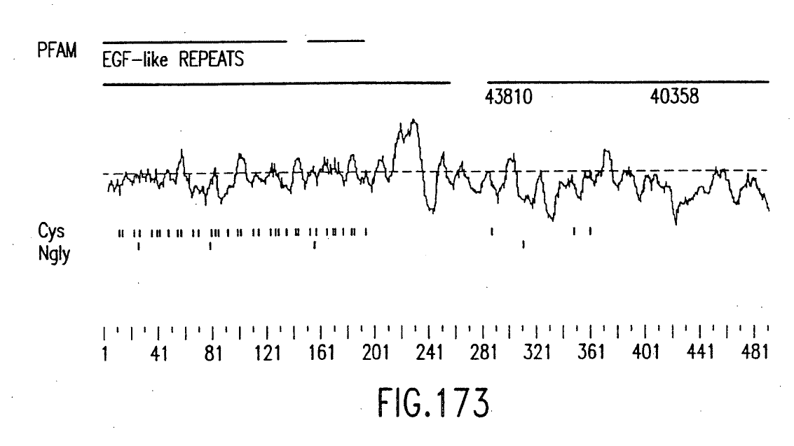

FIG. 173 depicts a hydropathy plot of Mouse TANGO 272. The hydropathy plot indicates the presence of a hydrophobic domain within Mouse TANGO 272, suggesting that mouse TANGO 272 is a transmembrane protein.

A cDNA encoding rat TANGO 272 was identified by analyzing the sequences of clones present in a rat neonatal sciatic nerve cDNA library.

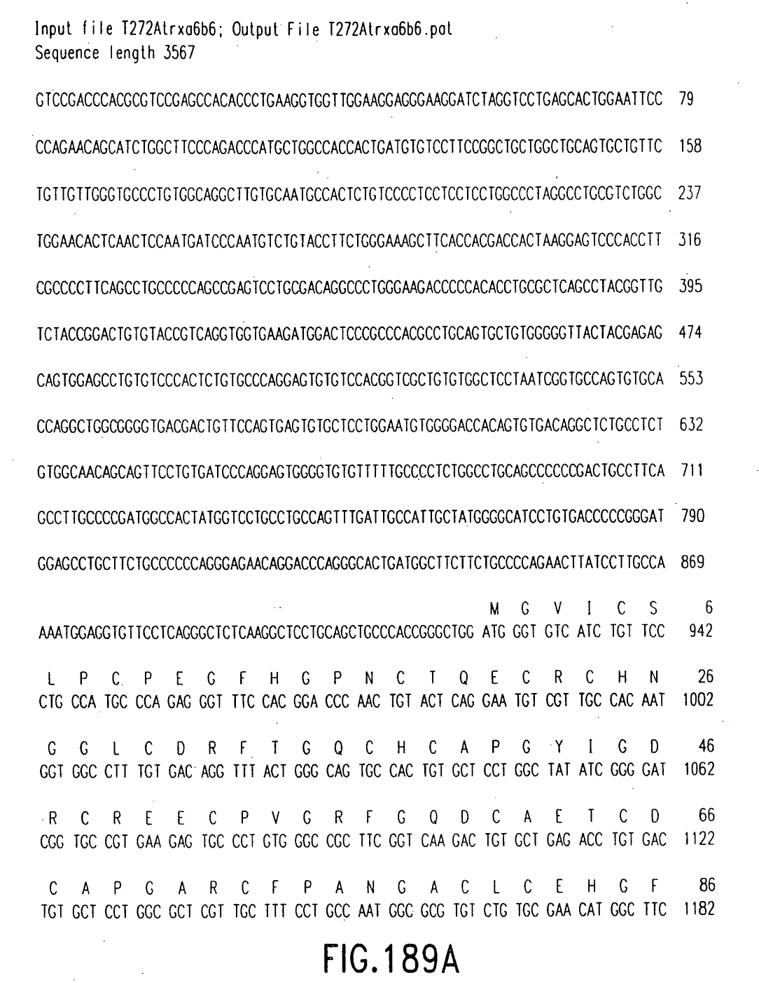

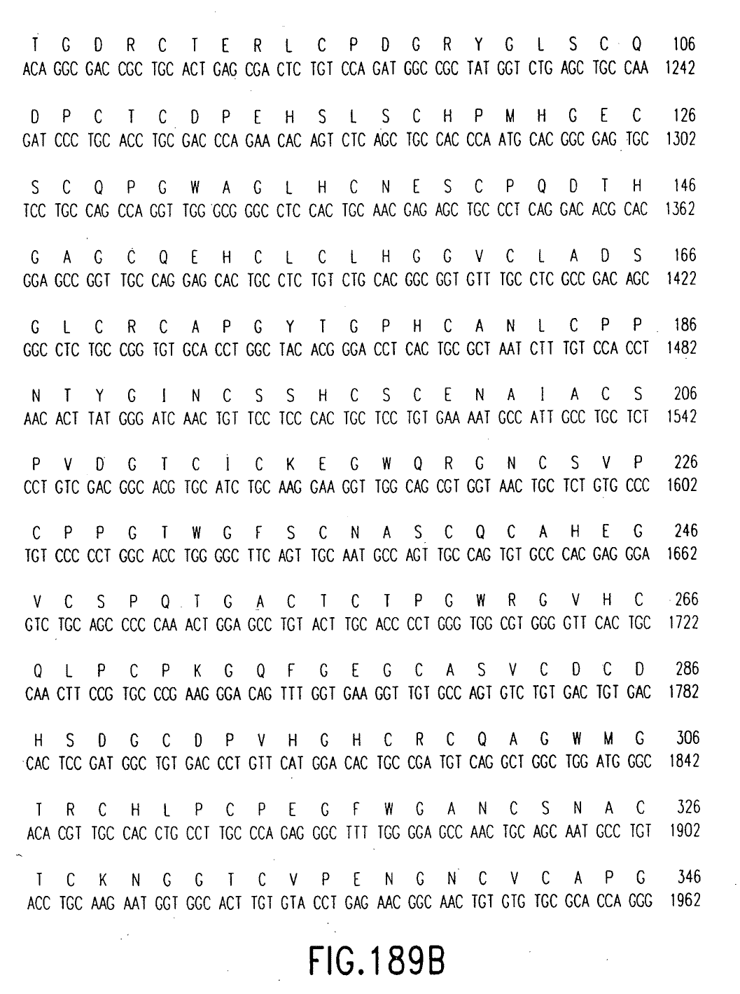

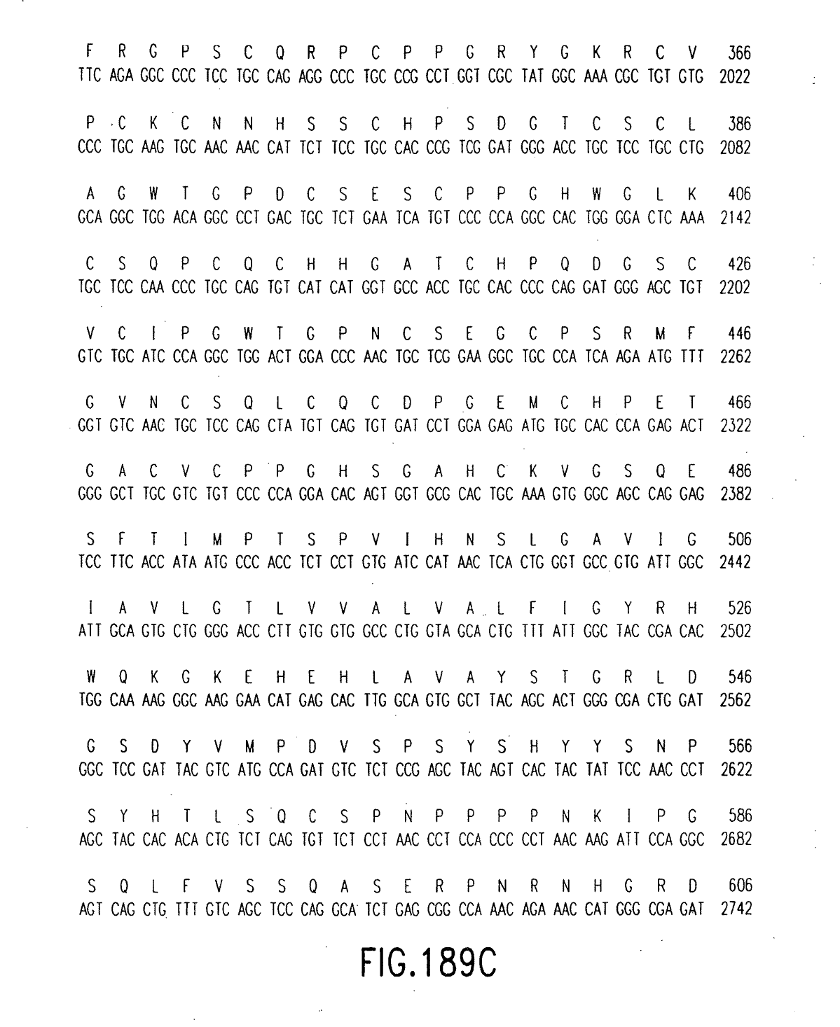

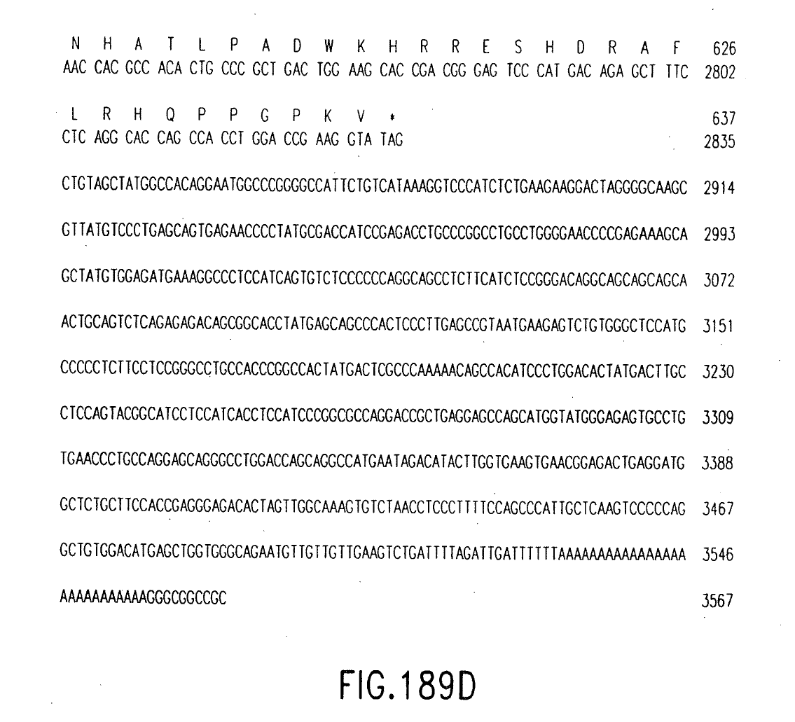

This analysis led to the identification of a clone, atrxa6b6, encoding partial rat TANGO 272. The cDNA of this clone is 3567 nucleotides long (FIG. 189A-189D; SEQ ID NO: 123). The 1908 nucleotide open reading frame of this cDNA (nucleotides 925-2832 of SEQ ID NO: 123) encodes a 636 amino acid protein (SEQ ID NO:124).

Rat TANGO 272 that has not been post-translationally modified is predicted to have a molecular weight of 67.4 kDa.

Rat TANGO 272 is a transmembrane protein having an extracellular domain which extends from about amino acid 1 to about amino acid 500, a transmembrane domain which extends from about amino acid 501 to about amino acid 524, and a cytoplasmic domain which extends from about amino acid 525 to amino acid 636 of SEQ ID NO:124.

Alternatively, in another embodiment, a rat TANGO 272 protein contains an extracellular domain which extends from about amino acid 525 to amino acid 636, a transmembrane domain which extends from about amino acid 501 to about amino acid 524, and a cytoplasmic domain which extends from about amino acid 1 to about amino acid 500 of SEQ ID NO:124.

Rat TANGO 272 includes eleven EGF-like domains at about amino acids 18-48; amino acids 61-91; amino acids 105-137; amino acids 150-180; amino acids 193-223; amino acids 236-266; amino acids 279-309; amino acids 322-352; amino acids 365-394; amino acids 407-437; and amino acids 450-480. FIG. 197A-197D depicts alignments of each of the EGF-like-domains of rat TANGO 272 with consensus hidden Markov model EGF-like domains.

Rat TANGO 272 further includes eleven laminin/EGF-like domains at about amino acids 22-61; amino acids 65-105; amino acids 109-150; amino acids 154-193; amino acids 197-236; amino acids 240-279; amino acids 283-322; amino acids 326-365; amino acids 368-407; amino acids 411-450; and amino acids 454-489 of SEQ ID NO: 124. FIG. 197A-197D depicts alignments of each of the laminin/EGF-like-domains of rat TANGO 272 with consensus hidden Markov model EGF-like domains.

Rat TANGO 272 further includes a delta serrate ligand domain from amino acids 246 to 309 of SEQ ID NO: 124. An alignment of the delta serrate ligand domain of rat TANGO 272 with a consensus hidden Markov model of this domain is also depicted in FIG. 197C.

Based on the Prosite analysis, EGF-like domain cysteine pattern signature are present at amino acids 37-48, 80-91, 126-137, 169-180, 255-266, 298-309, 341-352, 383-394, 426-437, and 469-480.

N-glycosylation sites are present at amino acids 17-20, 138-141, 192-195, 222-225, 237-240, 321-324, 372-375, 436-439, and 449-452. A cAMP/cGMP-dependent protein kinase phosphorylation site is present at amino acids 618-621. An amidation site is present at amino acids 361-364. Protein kinase C phosphorylation sites are present at amino acids 92-94, 327-329, 542-544, and 596-598. Casein kinase II phosphorylation sites are present at amino acids 104-107, 206-209, 281-284, and 390-393. A tyrosine kinase phosphorylation site is present at amino acids 94-101. N-myristylation sites are present at amino acids 2-7, 35-40, 58-63, 78-83, 134-139, 160-165, 167-172, 190-195, 210-215, 253-258, 319-324, 339-344, 381-386, 404-409, 424-429, 447-452, 483-488, and 502-507.

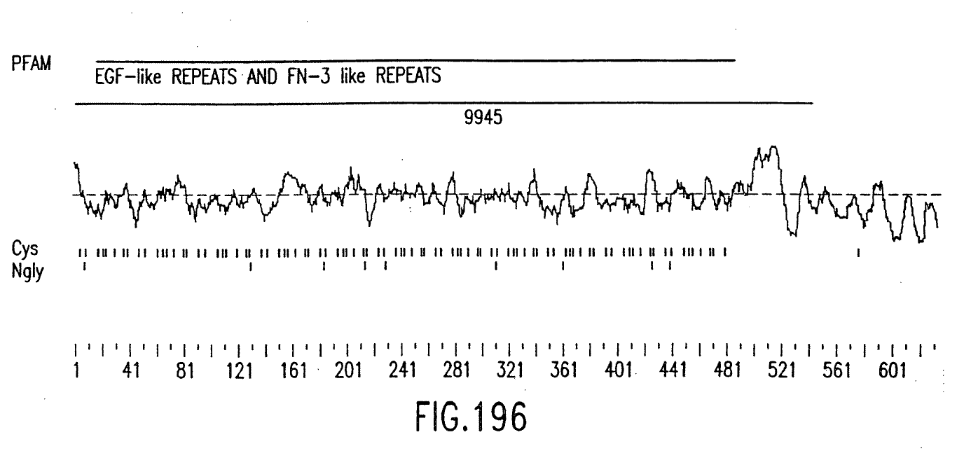

FIG. 196 depicts a hydropathy plot of rat TANGO 272. The hydropathy plot indicates the presence of a hydrophobic domain within rat TANGO 272, suggesting that rat TANGO 272 is a transmembrane protein.

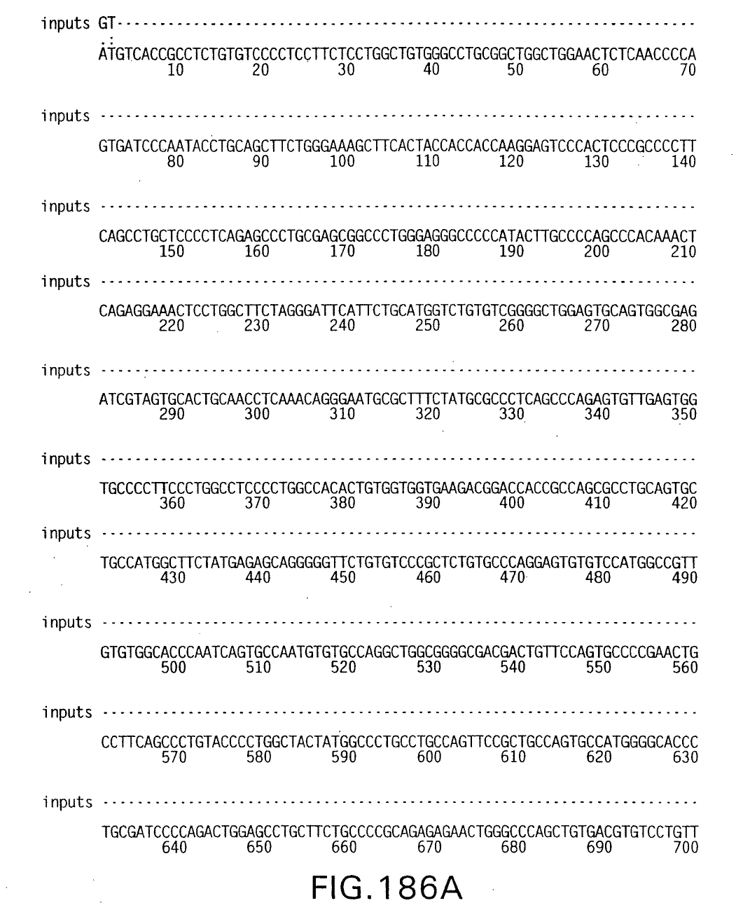

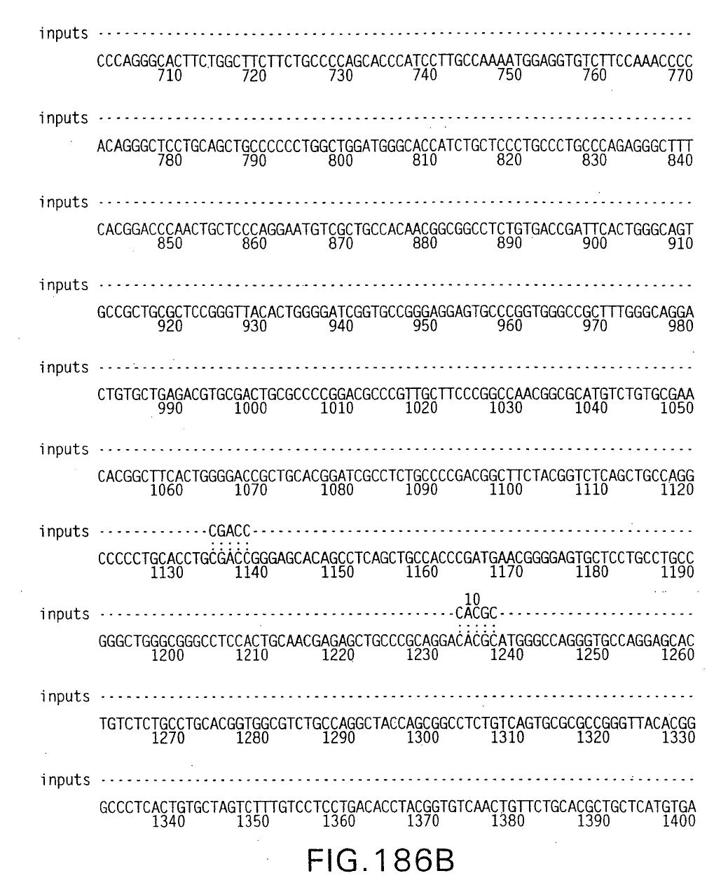

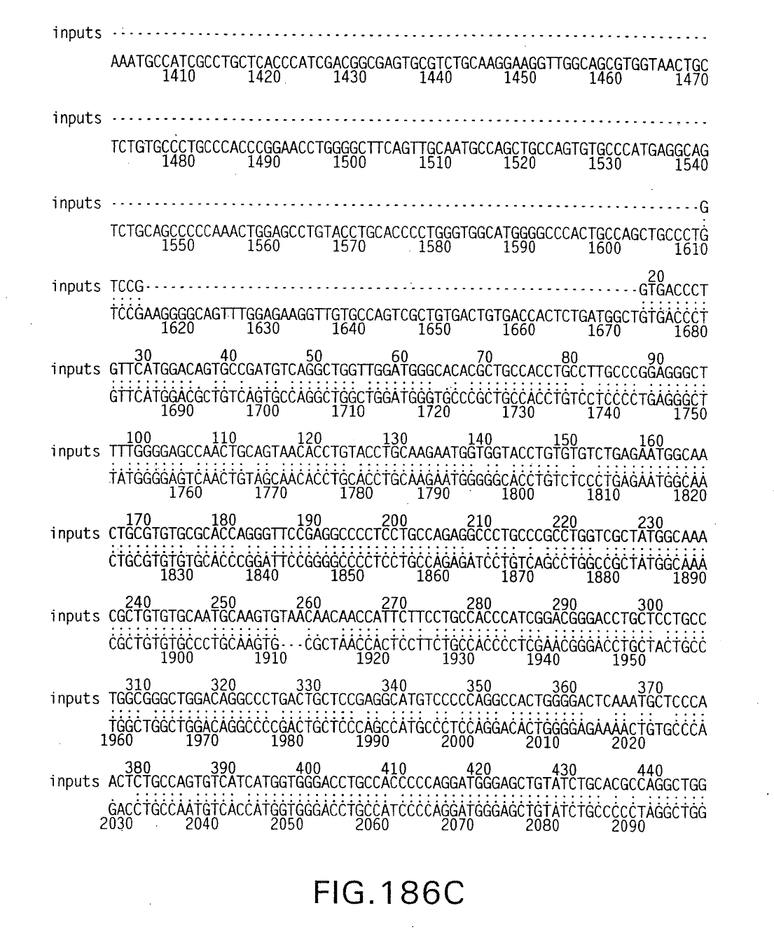

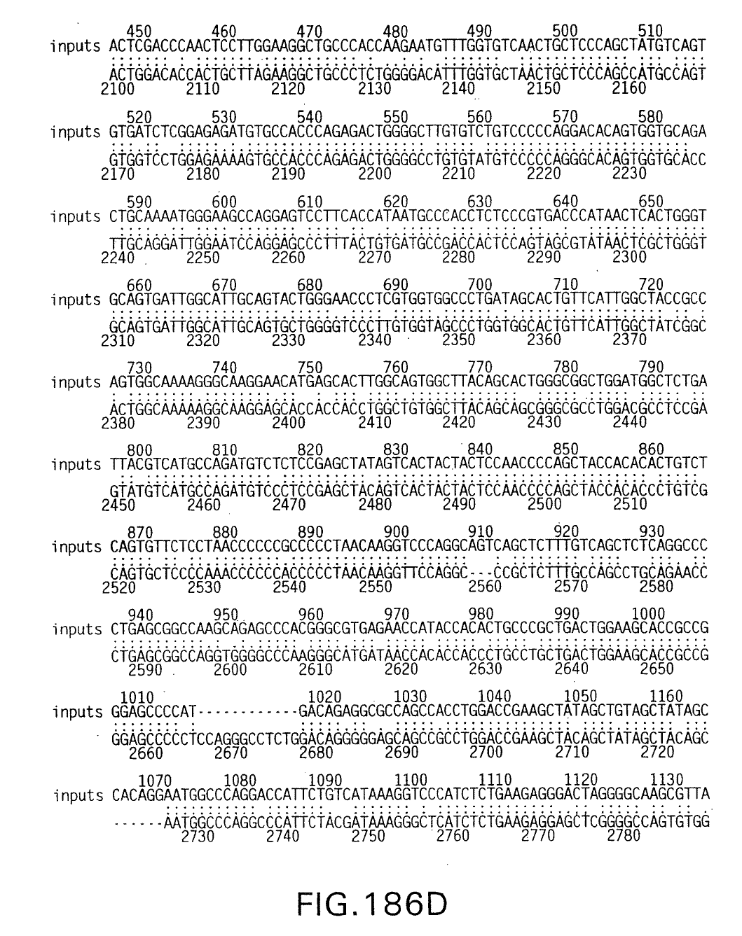

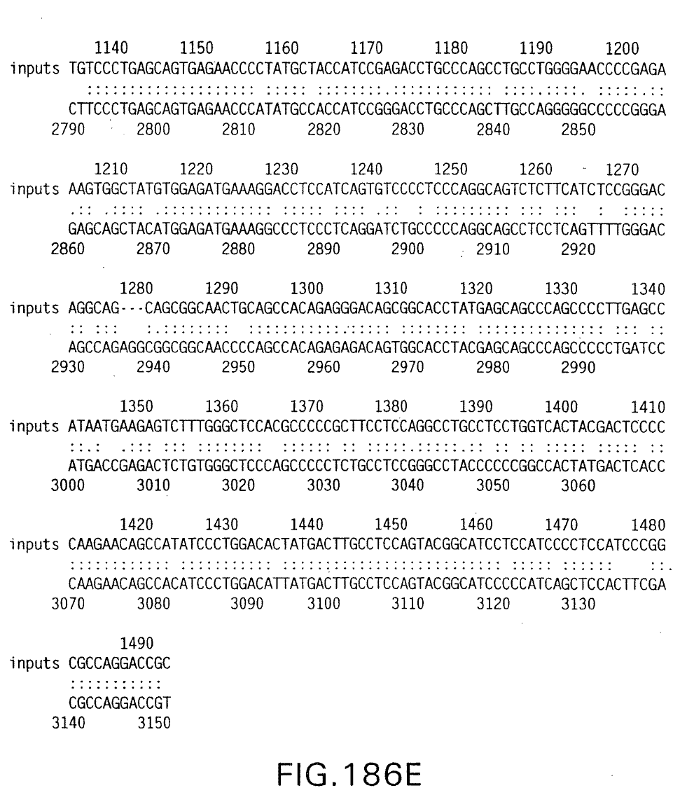

A global alignment between the nucleotide sequence of the open reading frame (ORF) of human TANGO 272 and the nucleotide sequence of the open reading frame of mouse TANGO 272 revealed a 39.1% identity (FIG. 186A-186E). The global alignment was performed using the ALIGN program version 2.0u (Matrix file used: pam 120.mat, gap penalties of −12/−4 with a global alignment score of −79; Myers and Miller, 1989, CABIOS4:11-7).

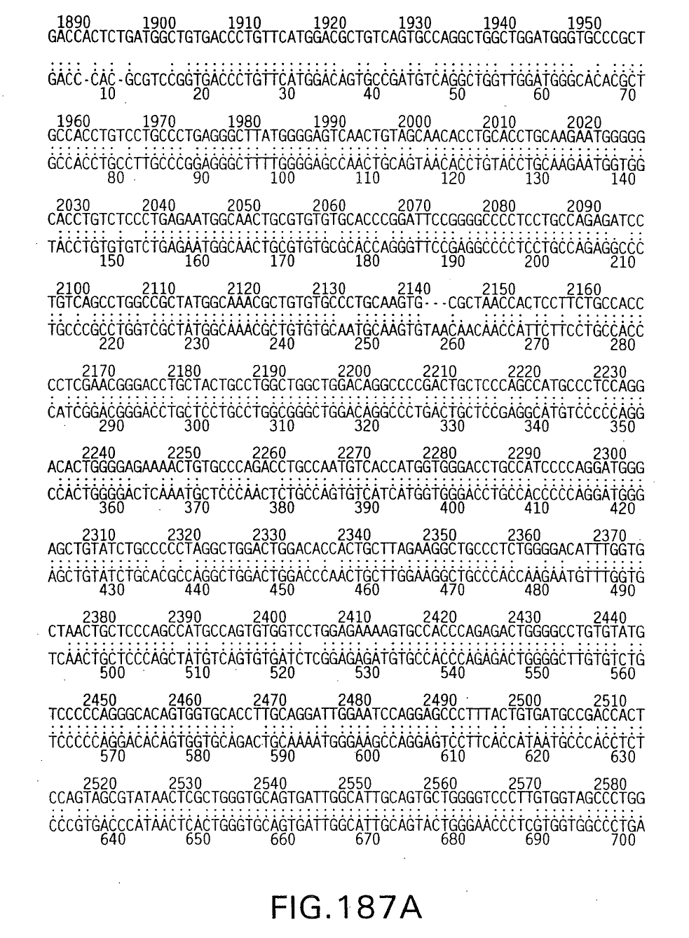

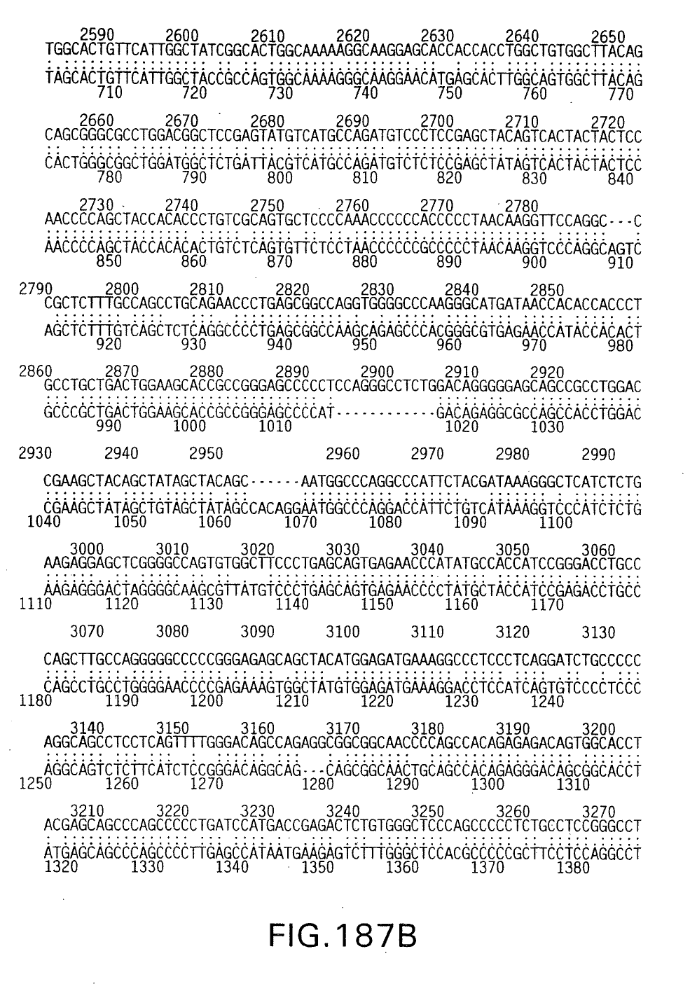

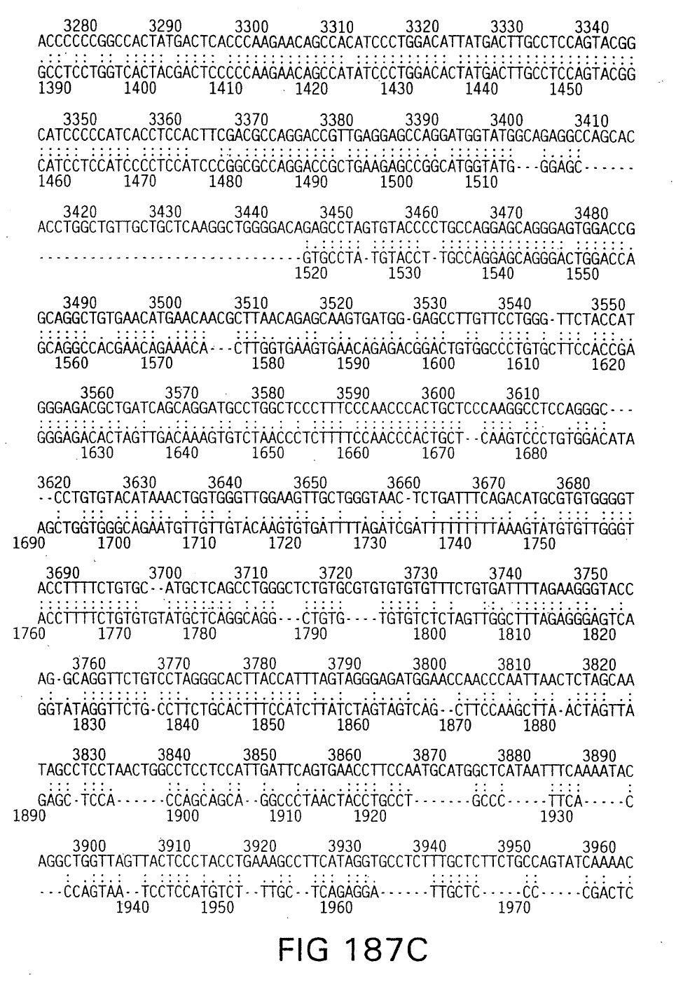

A local alignment between the nucleotide sequence of human TANGO 272 and the nucleotide sequence of mouse TANGO 272 revealed 67.6% identity over nucleotides 1890-4610 of the human TANGO 272 sequence (nucleotides 10-2560 of mouse TANGO 272) (FIG. 187A-187C). The local alignment was performed using the L-ALIGN program version 2.0u54 Jul. 1996 (Matrix file used: pam 120.mat, gap penalties of −12/−4 with a score of 8462; Huang and Miller, 1991, Adv. Appl. Math. 12:373-81).

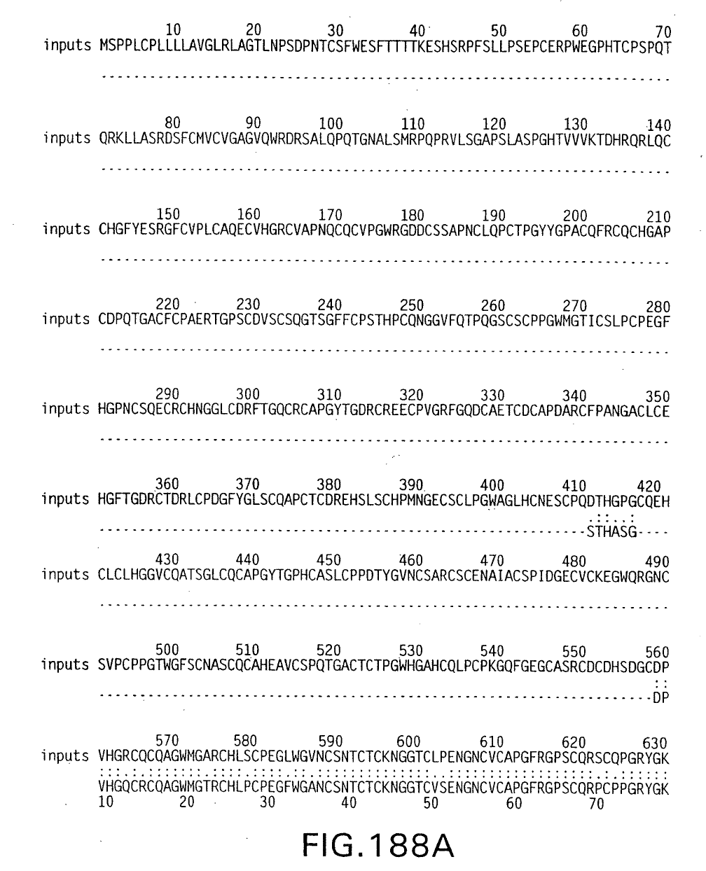

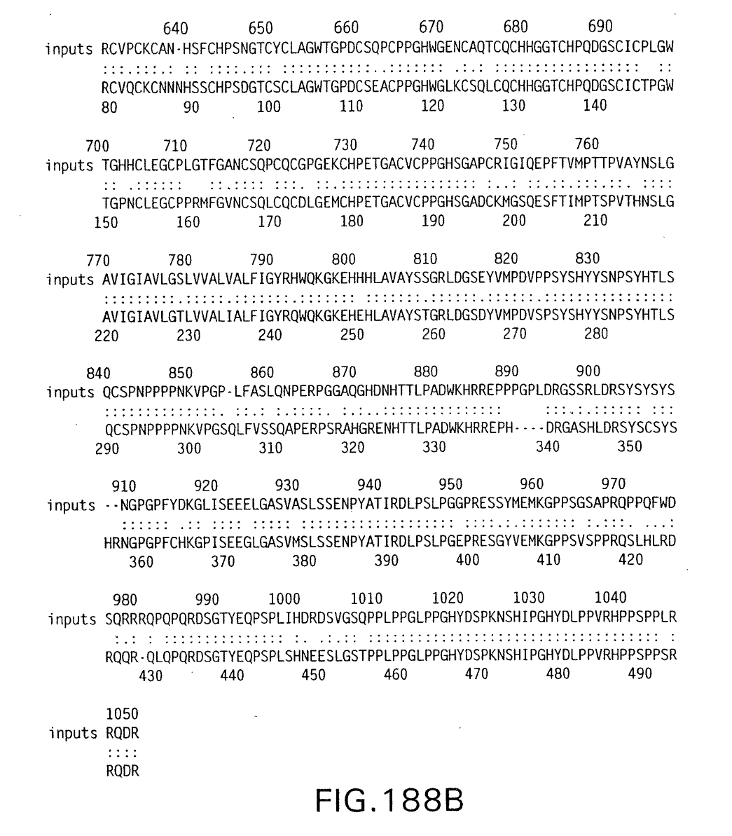

A global alignment between the amino acid sequence of human TANGO 272 and the amino acid sequence of mouse TANGO 272 revealed a 38.2% identity (FIG. 188A-188B). The global alignment was performed using the ALIGN program version 2.0u (Matrix file used: pam 120.mat, gap penalties of −12/−4 with a global alignment score of 15-19; Myers and Miller, 1989, CABIOS 4:11-7).

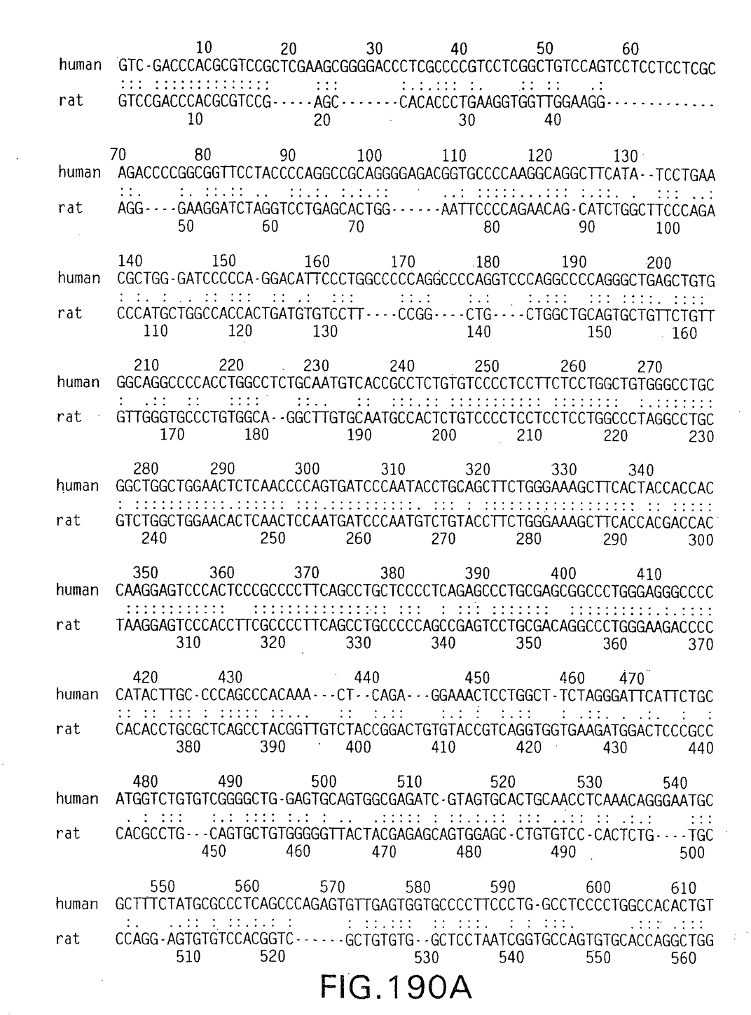

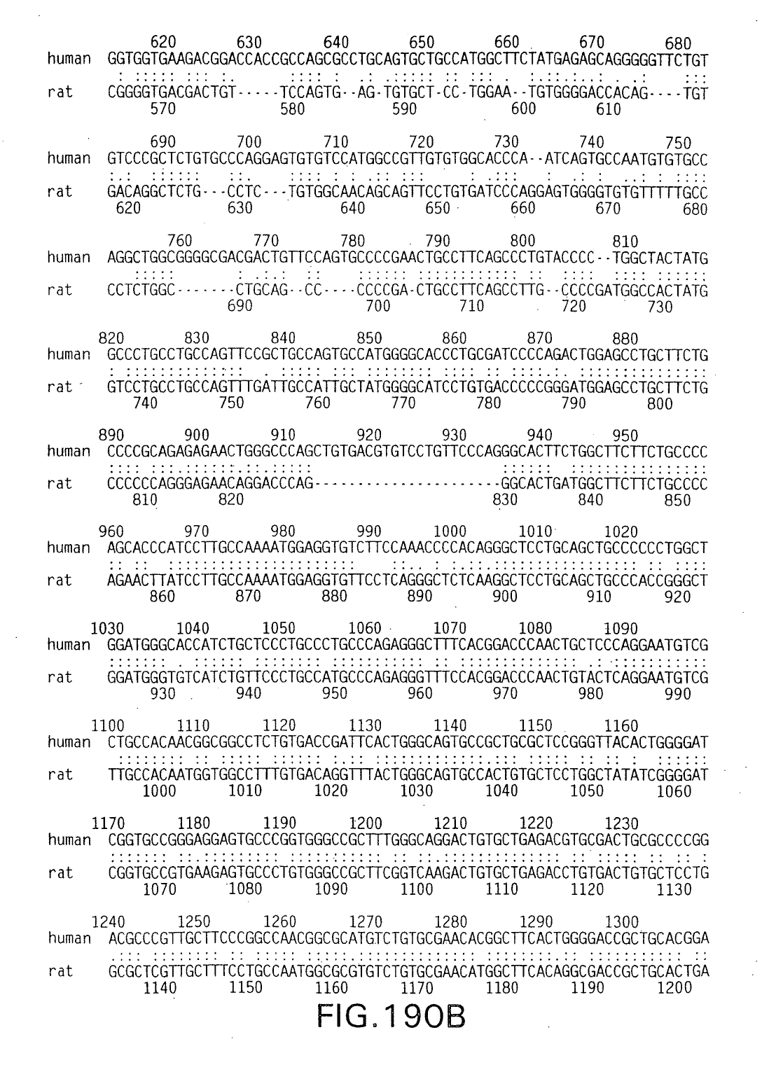

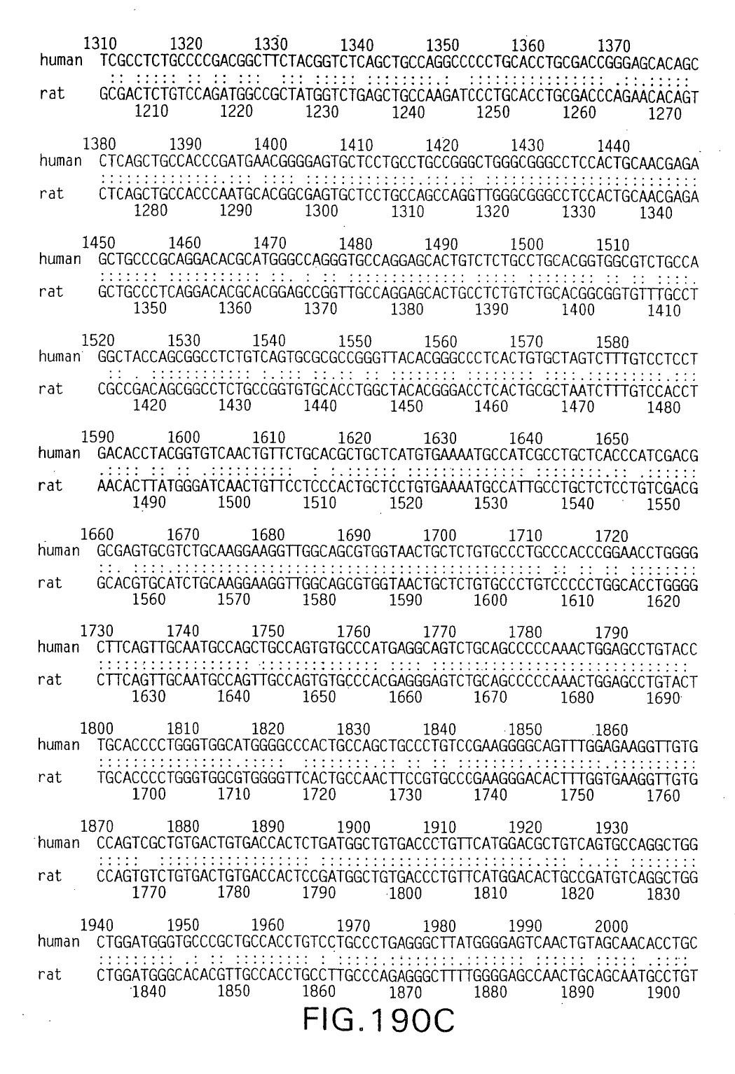

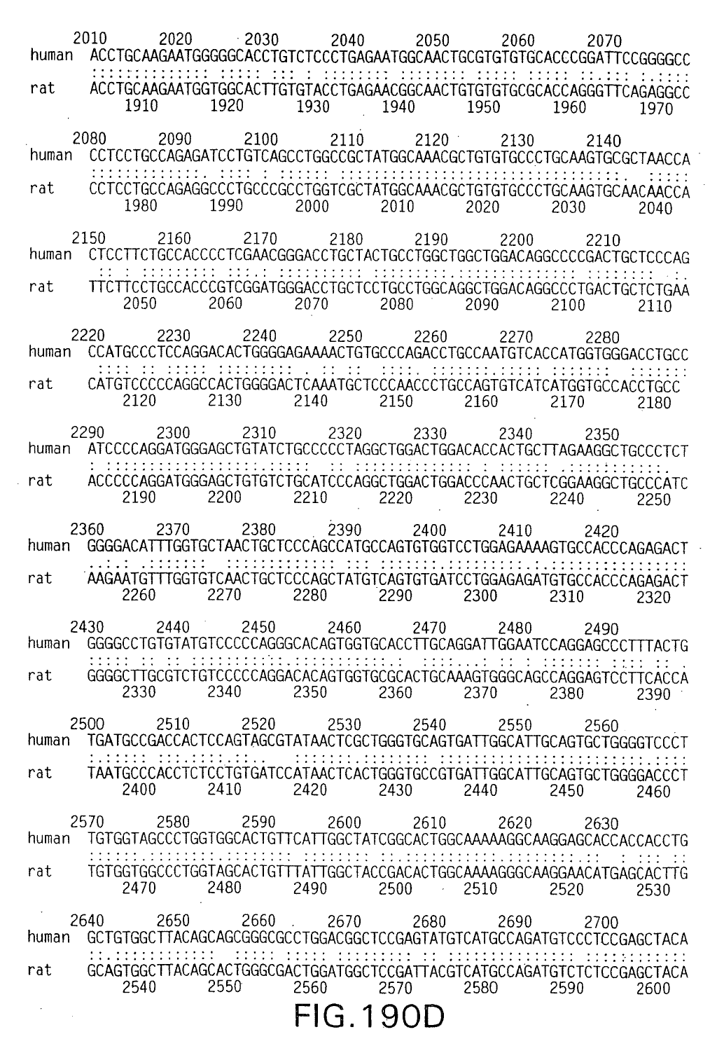

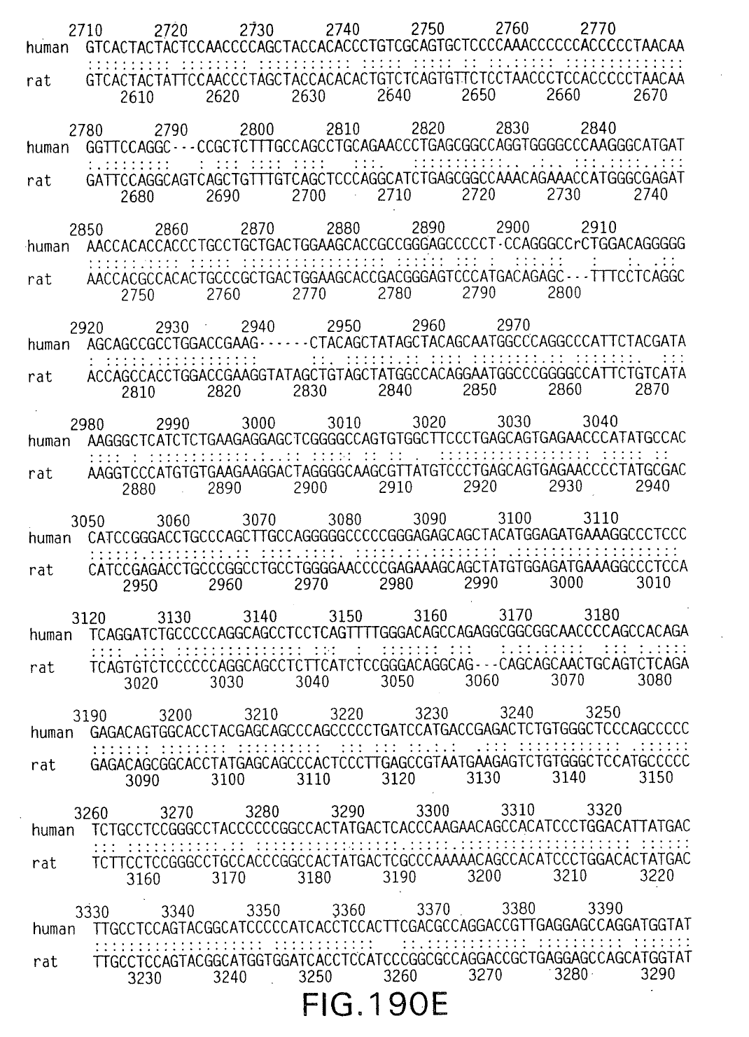

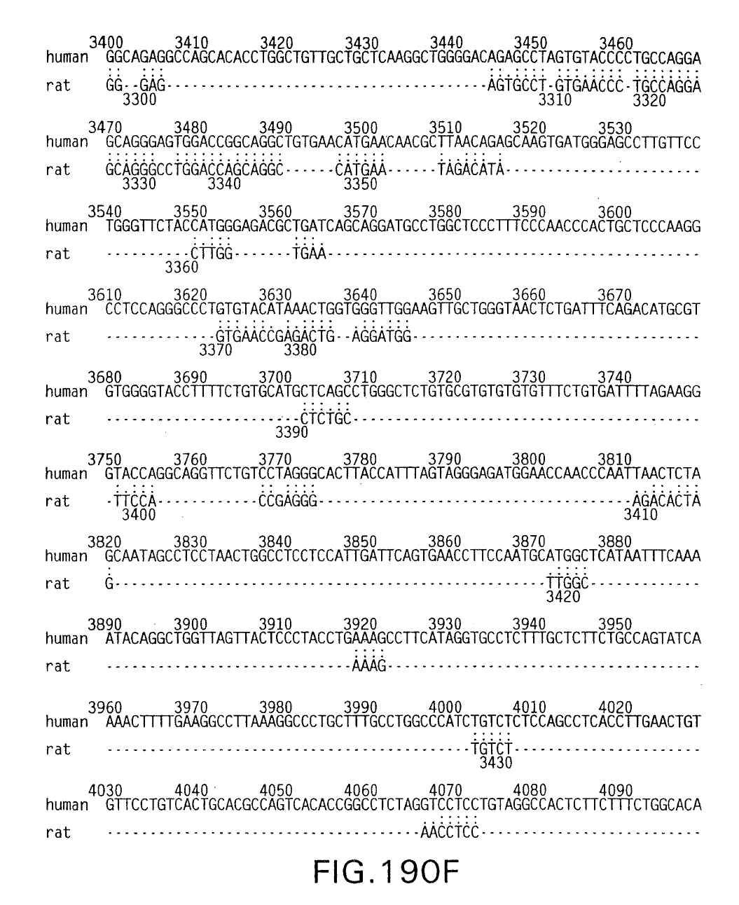

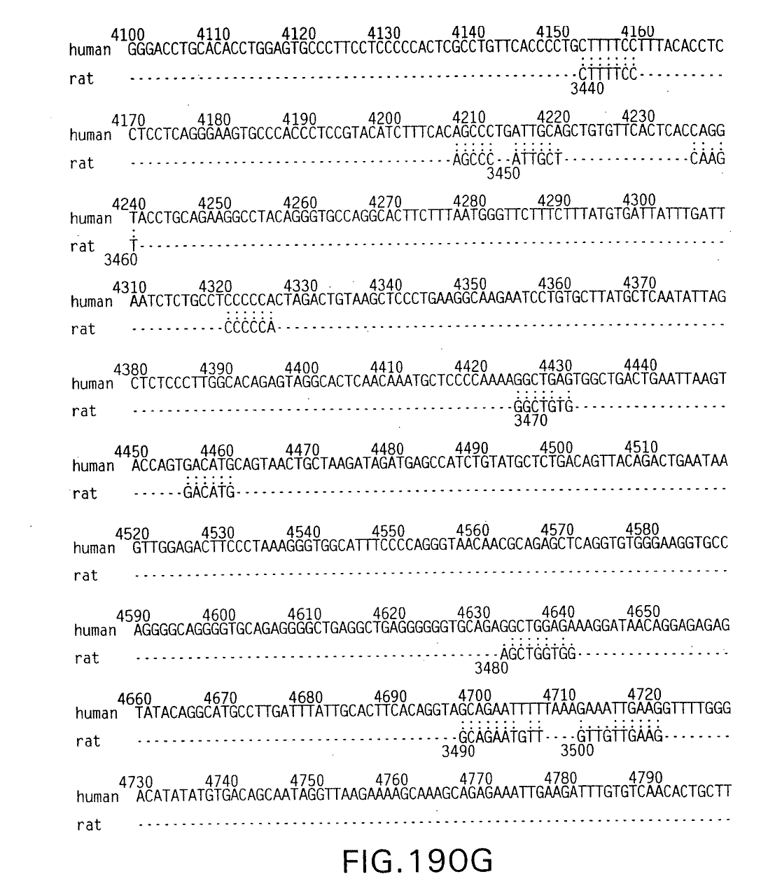

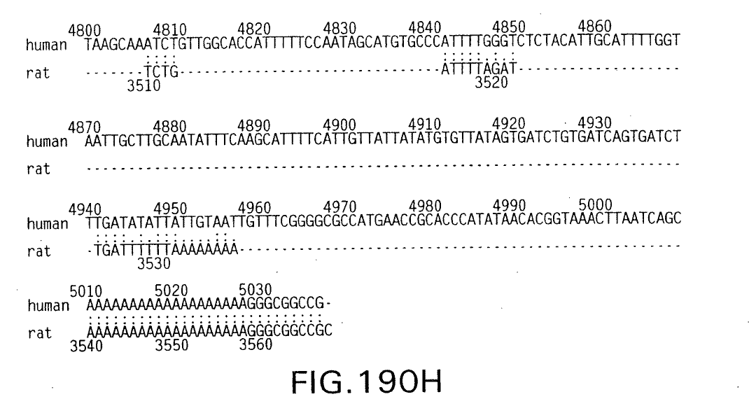

A global alignment between the nucleotide sequence of human TANGO 272 and the nucleotide sequence of rat TANGO 272 revealed a 55.7% identity (FIG. 190A-190H). The global alignment was performed using the ALIGN program version 2.0u (Matrix file used: pam 120.mat, gap penalties of −12/−4 with a global alignment score of 8635; Myers and Miller, 1989, CABIOS 4:11-7).

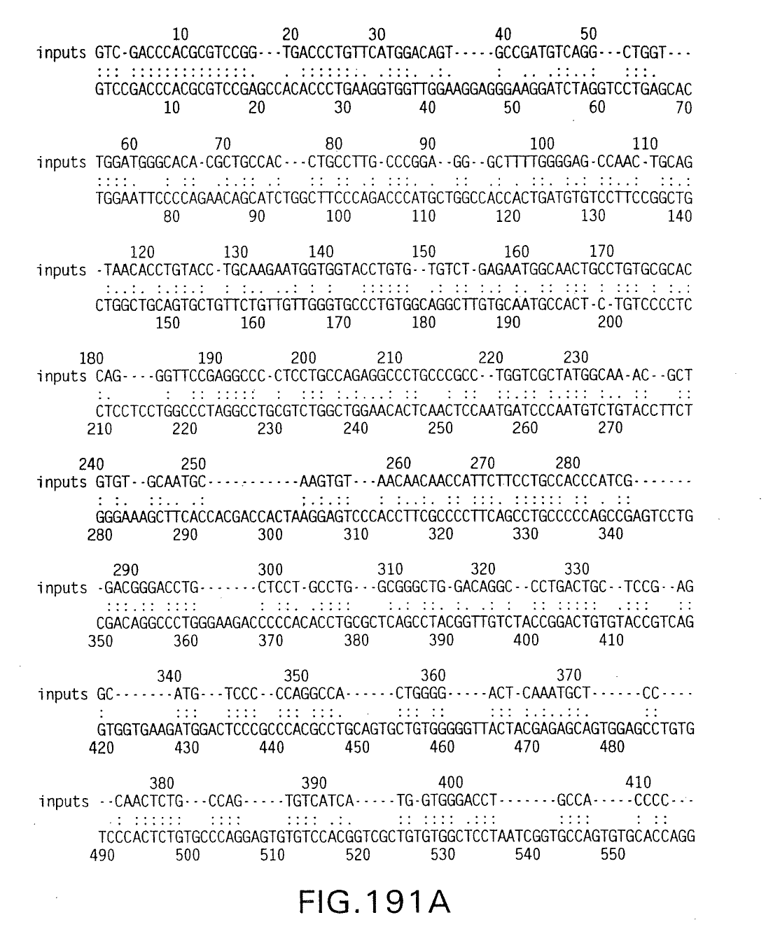

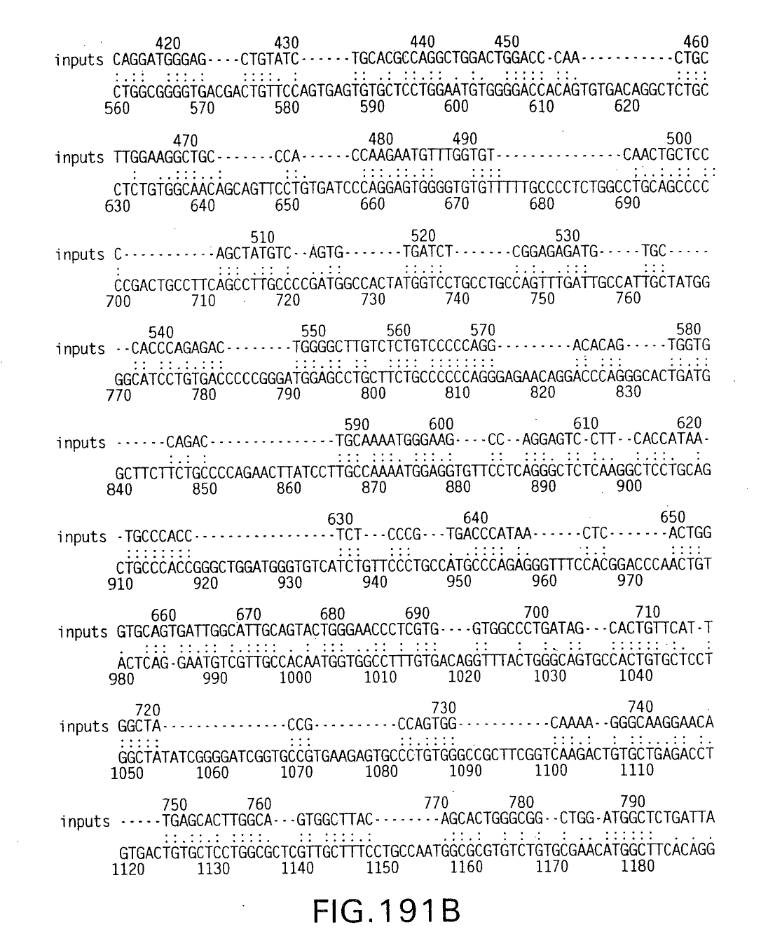

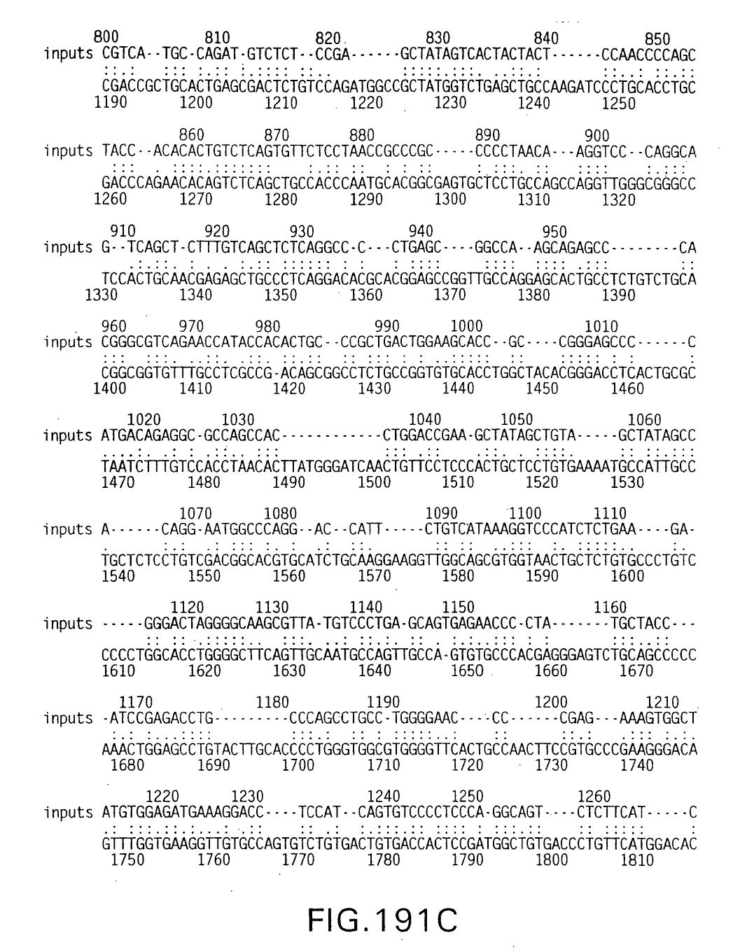

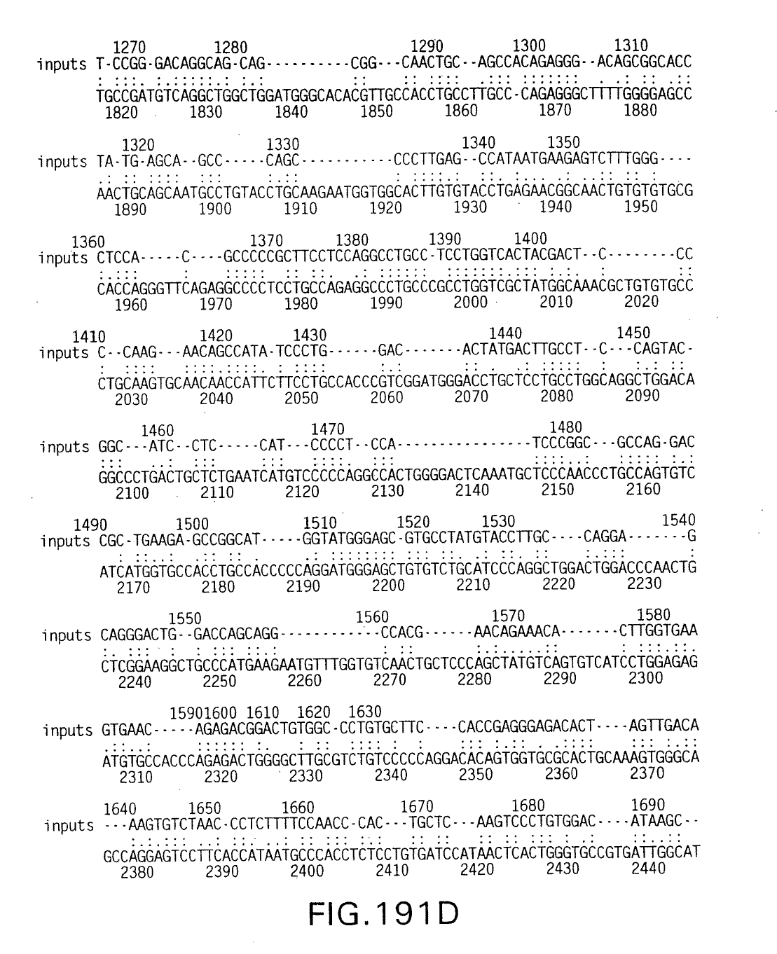

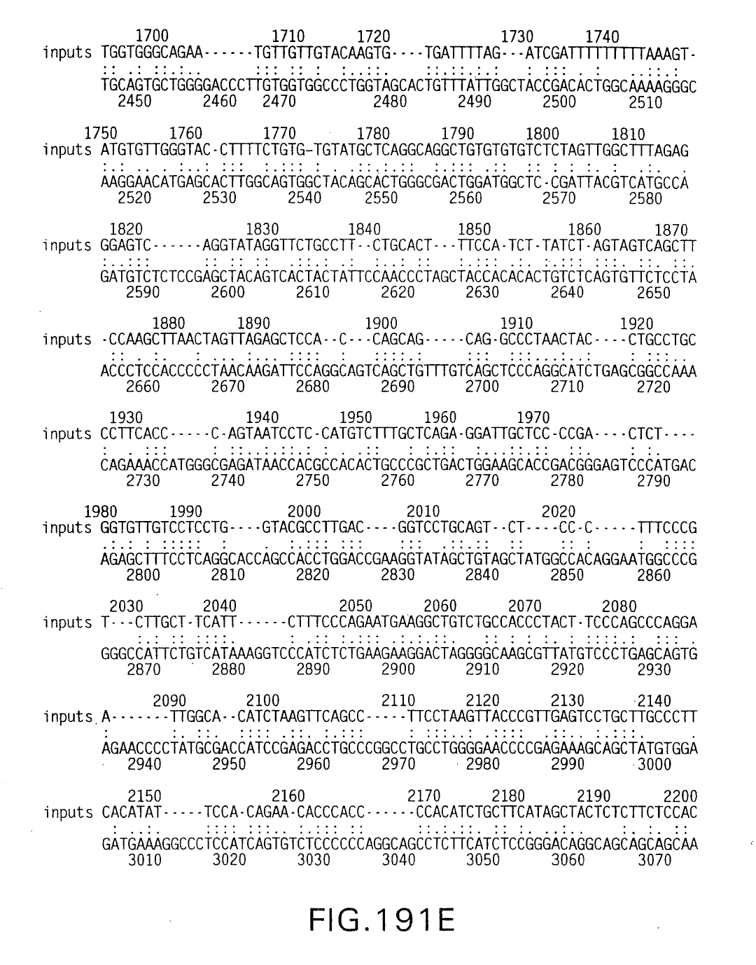

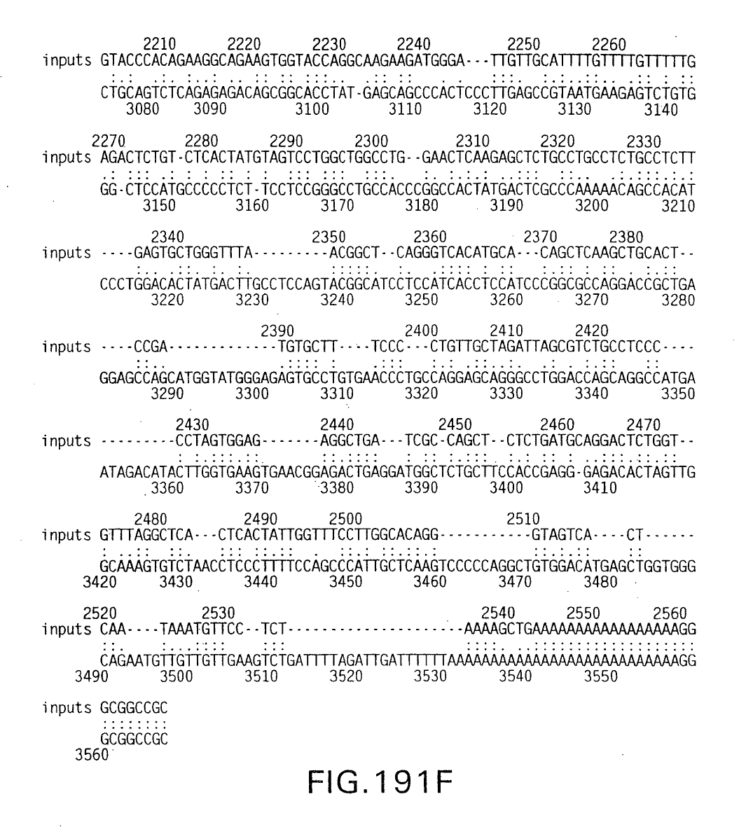

A global alignment between the nucleotide sequence of mouse TANGO 272 and the nucleotide sequence of rat TANGO 272 revealed a 43.7% identity (FIG. 191A-191F). The global alignment was performed using the ALIGN program version 2.0u (Matrix file used: pam 120.mat, gap penalties of −12/−4 with a global alignment score of 2827; Myers and Miller, 1989, CABIOS 4:11-7).

### Use of TANGO 272 Nucleic Acids, Polypeptides, and Modulators Thereof

TANGO 272 includes fourteen EGF-like domains. Proteins having such domains play a role in mediating protein-protein interactions, and thus can influence a wide variety of biological processes, including cell surface recognition; modulation of cell-cell contact; modulation of cell fate determination; and modulation of wound healing and tissue repair.

TANGO 272 further includes an RGD cell attachment site. Proteins having such domains are typically extracellular matrix proteins such as collagens, laminin and fibronectin, among others (reviewed in Ruoslahti, 1996, Annu. Rev. Cell Dev. Biol. 12:697-715). An RGD cell attachment site typically interacts (e.g., binds to) a cell surface receptor, such as an integrin receptor, and thus mediates a variety of biological processes, including cellular adhesion, migration, among others.

Because TANGO 272 includes EGF-like domains and an RGD cell attachment site, TANGO 272 polypeptides, nucleic acids, and modulators thereof can be used to treat disorders involving, e.g., cellular migration, proliferation, and differentiation of a cell. For example, TANGO 272 polypeptides, nucleic acids, and modulators thereof can be used to treat neoplastic disorders, e.g., cancer, tumor metastasis.

TANGO 272 polypeptides, nucleic acids, and modulators thereof can be used to modulate function, survival, morphology, migration, proliferation, tissue repair and/or differentiation of cells in the tissues in which it is expressed (e.g., microvascular endothelial cells). For example, TANGO 272 polypeptides, nucleic acids, and modulators thereof can be used to modulate cardiovascular function, and/or to promote wound healing and tissue repair (e.g., of the skin, cornea and mucosal lining). Accordingly, these molecules can be used to treat a variety of cardiovascular diseases including, but not limited to, atherosclerosis, ischemia reperfusion injury, cardiac hypertrophy, hypertension, coronary artery disease, myocardial infarction, arrhythmia, cardiomyopathies, and congestive heart failure.

As TANGO 272 exhibits expression in the heart, TANGO 272 nucleic acids, proteins, and modulators thereof can be used to treat heart disorders as described herein.

In another example, TANGO 272 polypeptides, nucleic acids, or modulators thereof, can be used to treat placental disorders, such as toxemia of pregnancy (e.g., preeclampsia and eclampsia), placentitis, or spontaneous abortion.

In another example, TANGO 272 polypeptides, nucleic acids, or modulators thereof, can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another example, TANGO 272 polypeptides, nucleic acids, or modulators thereof, can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 272 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 272 polypeptides, nucleic acids, or modulators thereof, can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Pararnyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, TANGO 272 polypeptides, nucleic acids, or modulators thereof, can be used to treat renal disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, TANGO 272 polypeptides, nucleic acids, or modulators thereof, can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

Further, in light of TANGO 272's pattern of expression in humans, TANGO 272 expression can be utilized as a marker for specific tissues (e.g., cardiovascular) and/or cells (e.g., cardiac) in which TANGO 272 is expressed. TANGO 272 nucleic acids can also be utilized for chromosomal mapping.

### TANGO 295

A cDNA encoding human TANGO 295 was identified by analyzing the sequences of clones present in a human mammary epithelium cDNA library.

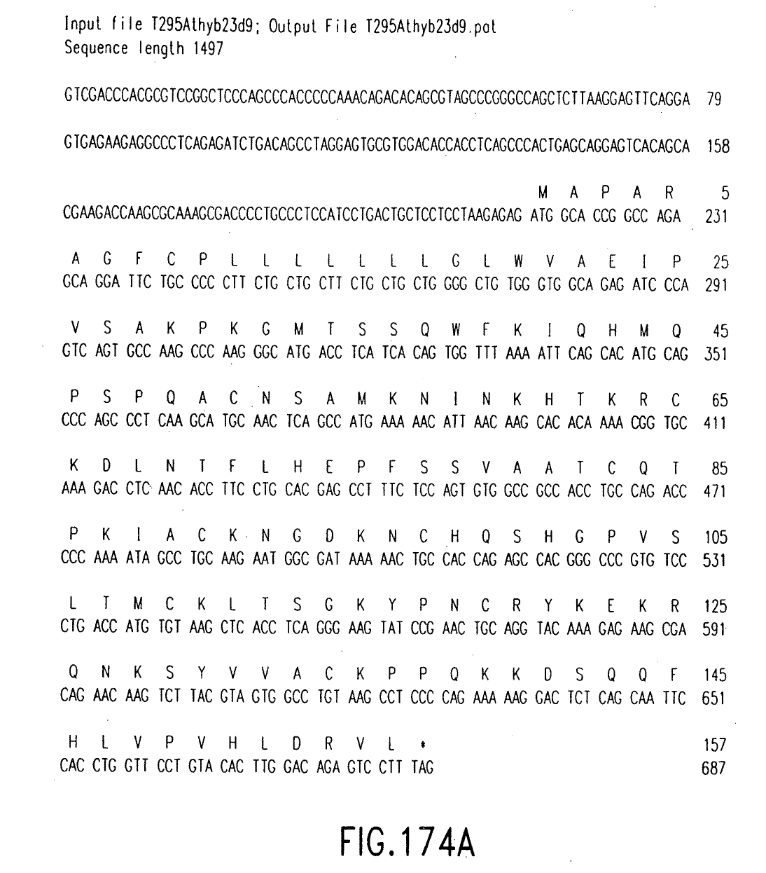

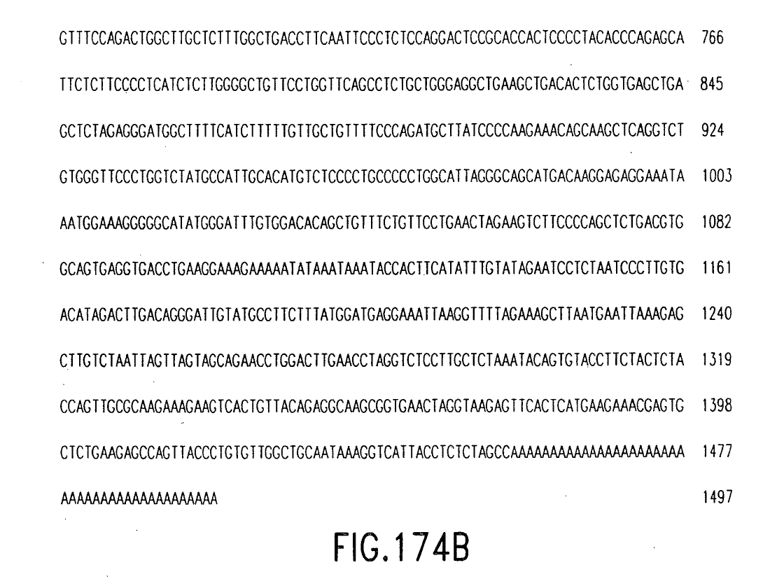

This analysis led to the identification of a clone, jthvb023d09, encoding full-length human TANGO 295. The cDNA of this clone is 1497 nucleotides long (FIG. 174A-174B; SEQ ID NO: 117). The 468 nucleotide open reading frame of this cDNA (nucleotides 217-684 of SEQ ID NO: 117) encodes a 156 amino acid protein (SEQ ID NO: 118).

The signal peptide prediction program SIGNALP (Nielsen et al., 1997, Protein Engineering 10:1-6) predicted that human TANGO 295 includes a 28 amino acid signal peptide at amino acid 1 to about amino acid 28 preceding the mature human TANGO 295 protein which corresponds to about amino acid 29 to amino acid 156.

Human TANGO 295 that has not been post-translationally modified is predicted to have a molecular weight of 17.5 kDa prior to cleavage of its signal peptide and a molecular weight of 14.6 kDa subsequent to cleavage of its signal peptide.

Secretion assays reveal that human TANGO 295 protein is secreted as a 17 kDa protein. The secretion assays were performed as follows: 8×10⁵ 293T cells were plated per well in a 6-well plate and the cells were incubated in growth medium (DMEM, 10% fetal bovine serum, penicillin/streptomycin) at 37° C., 5% CO₂ overnight. 293T cells were transfected with 2 μg of full-length MANGO 245 inserted in the pMET7 vector/well and 10 μg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16-424-54). 1 ml DMEM without methionine and cysteine with 50 μCi Trans-³⁵S (ICN Cat. # 51006) was added to each well and the cells were incubated at 37° C., 5% CO₂ for the appropriate time period. A 150 μl aliquot of conditioned medium was obtained and 150 μl of 2×SDS sample buffer was added to the aliquot. The sample was heat-inactivated and loaded on a 4-20% SDS-PAGE gel. The gel was fixed and the presence of secreted protein was detected by autoradiography.

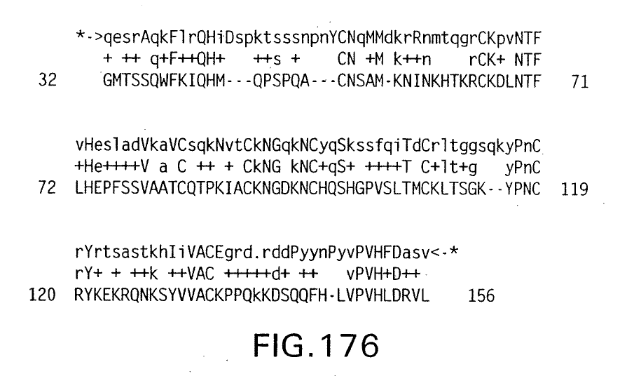

TANGO 295 family members can include a pancreatic ribonuclease domain sequence. As used herein, the term “pancreatic ribonuclease domain” includes an amino acid sequence having at least about 100 to 150, preferably 110-140, more preferably 120-130, and most preferably 124 amino acids in length. Preferably, a pancreatic ribonuclease domain further includes at least one, preferably two, three, four and most preferably five conserved cysteine residues and an amino acid residue, e.g. a lysine, which is involved in catalytic activity. Preferably, at least one cysteine residue is involved in a disulfide bond, a lysine residue is involved in catalytic activity, and three other residues involved in substrate binding. Proteins having the pancreatic ribonuclease domain are pyrimidine-specific endonucleases present in high quantities in the pancreas of a number of mammalian taxa and of a few reptiles. The pancreatic ribonuclease domain of TANGO 295 extends from about amino acid 32 to amino acid 156 of SEQ ID NO: 118. FIG. 176 depicts an alignment of the consensus hidden Markov model pancreatic ribonuclease domain with this domain in human TANGO 295 at amino acids 32 to 156 of SEQ ID NO: 118.

Human TANGO 295 includes a pancreatic ribonuclease domain at amino acids 32-156. FIG. 176 depicts an alignment of pancreatic ribonuclease domain of human TANGO 295 with a consensus hidden Markov model pancreatic ribonuclease domain.

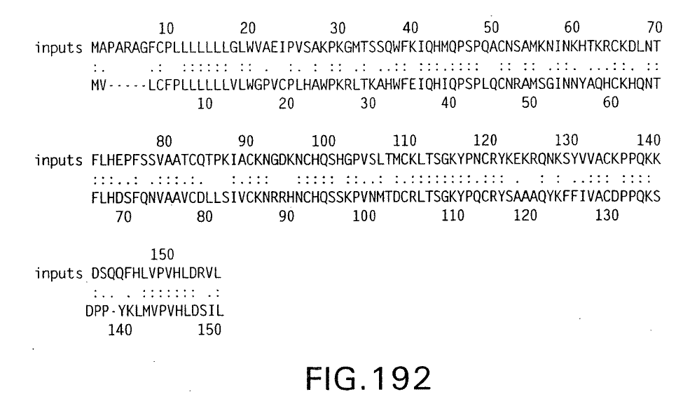

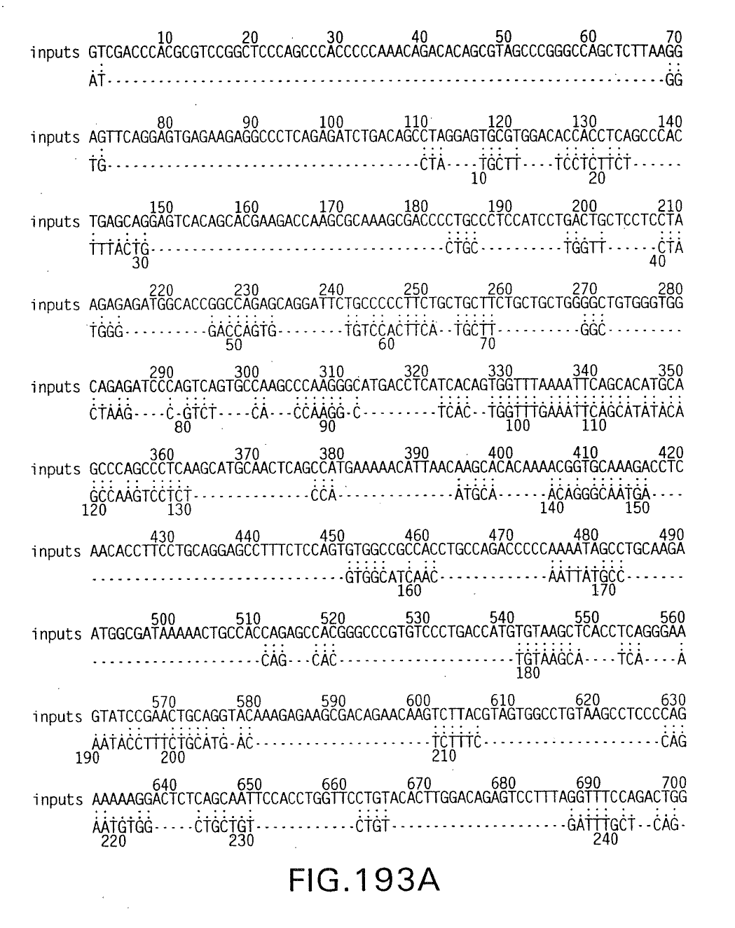

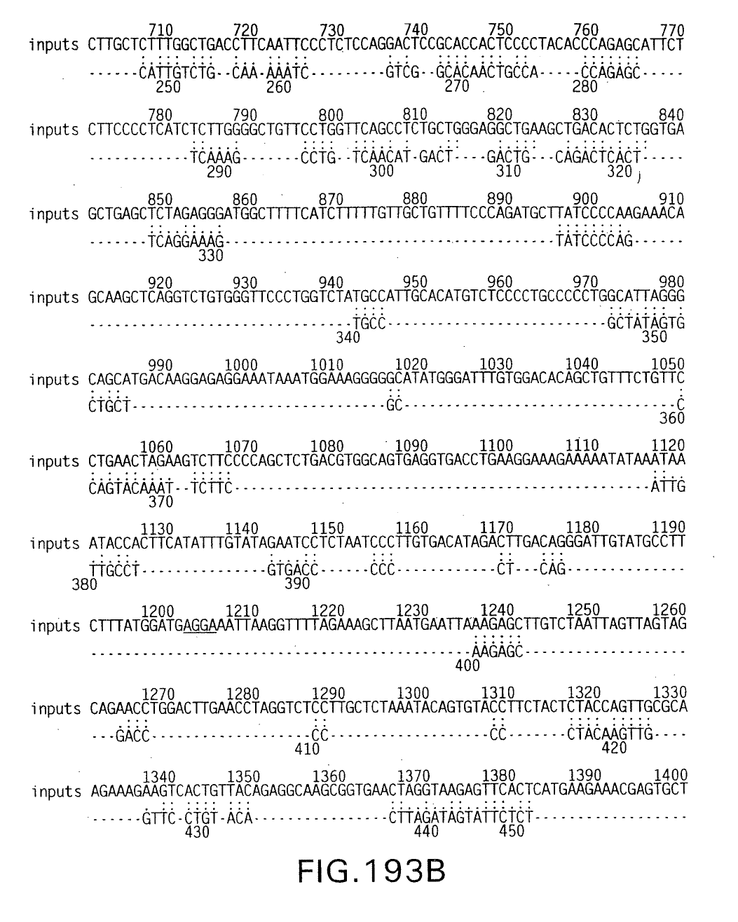

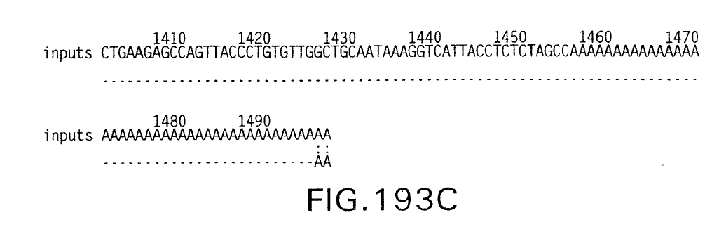

An N-glycosylation site is present at amino acids 127-130. A cAMP/cGMP dependent protein kinase site is present at amino acids 139-142. Protein kinase C phosphorylation sites are present at amino acids 27-29, 62-64, 85-87, and 113-115. N-myristylation sites are present at amino acids 18-23, and 32-37. Global alignment of the human TANGO 295 and GenPept AF037081 amino acid sequences revealed 53.2% identity (Matrix file used: pam 120.mat, gap penalties of −12/−4; Myers and Miller, 1989, CABIOS 4:11-7) (FIG. 192). A global alignment of the human TANGO 295 and GenPept AF037081 nucleotide sequences revealed a 22.6% identity between these two sequences (FIG. 193A-193C) (Matrix file used: pam 120.mat, gap penalties of −12/−4 with a global alignment score of −2718; Myers and Miller, 1989, CABIOS 54:11-7).

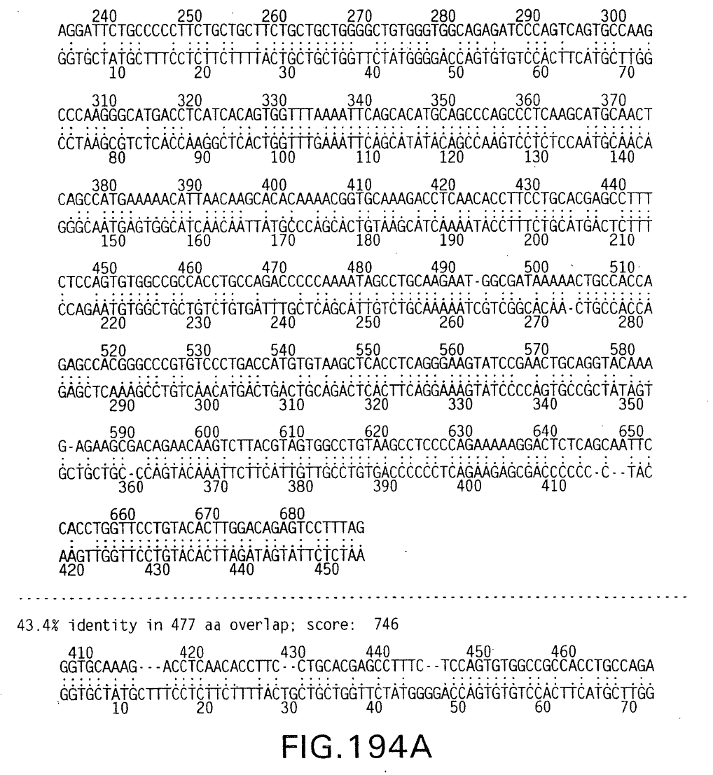

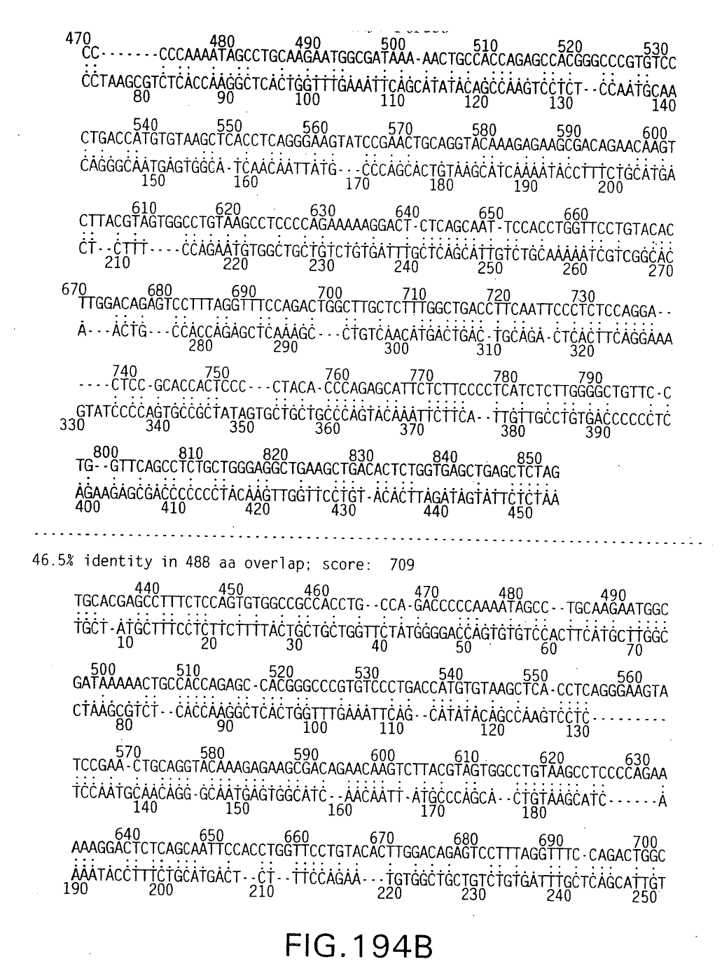

Local alignment of the human TANGO 295 and Genbank AF037081 nucleotide sequences revealed 62.7% identity between nucleotides 235-687 of human TANGO 295, and nucleotides 3-453 of AF037081; 43.4% identity between nucleotides 410-850 of human TANGO 295, and nucleotides 3450 of AF037081; and 46.5% identity between nucleotides 432-700 of human TANGO 295, and nucleotides 5-251 of AF037081 (Matrix file used: pam 120.mat, gap penalties of −12/−4 with a global alignment score of 1214; Huang and Miller, 1991, Adv. Appl. Math. 12:373-81) (FIG. 194A-194B).

Clone jthvb023d09, which encodes human TANGO 295, was deposited as a composite deposit having a designation EpT295 with the American Type Culture Collection (ATCCD 10801 University Boulevard, Manassas, Va. 20110-2209) on Jun. 18, 1999 and assigned Accession Number PTA-249. Deposit conditions are described below in the section entitled “Deposit of Clones”. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

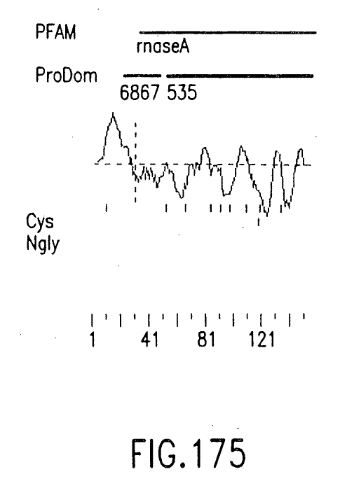

FIG. 175 depicts a hydropathy plot of human TANGO 295. The hydropathy plot indicates that human TANGO 295 has a signal peptide at its amino terminus, suggesting that human TANGO 295 is a secreted protein.

### Use of TANGO 295 Nucleic Acids, Polypeptides, and Modulators Thereof

TANGO 295 includes a pancreatic ribonuclease domain. Proteins having such domains have pyrimidine-specific endonuclease activity, and are present at elevated levels in the pancreas of various mammals and few reptiles. TANGO 295 shows some structural similarities to Ribonuclease k6 (RNase k6). RNase k6 is expressed in human monocytes and monophils (but not in eosinophils), suggesting a role for this ribonuclease in regulating host defense. Based on the structural similarities between TANGO 295 and RNase k6, TANGO 295 may play a role in regulating host defense.

TANGO 295 polypeptides, nucleic acids, and modulators thereof, can be used to modulate the function, morphology, proliferation and/or differentiation of cells in the tissues in which it is expressed (e.g., mammary epithelium). Accordingly, TANGO 295 polypeptides, nucleic acids, and modulators thereof can be used to treat epithelial disorders, e.g., mammary epithelial disorders (e.g., breast cancer).

Further, in light of TANGO 295's presence in a human mamary epithelium cDNA library, TANGO 295 expression can be utilized as a marker for specific tissues (e.g., breast) and/or cells (e.g., mammary) in which TANGO 295 is expressed. TANGO 295 nucleic acids can also be utilized for chromosomal mapping.

### TANGO 354

A cDNA encoding human TANGO 354 was identified by analyzing the sequences of clones present in a Mixed Lyrnphocyte Reaction (MLR) cDNA library.

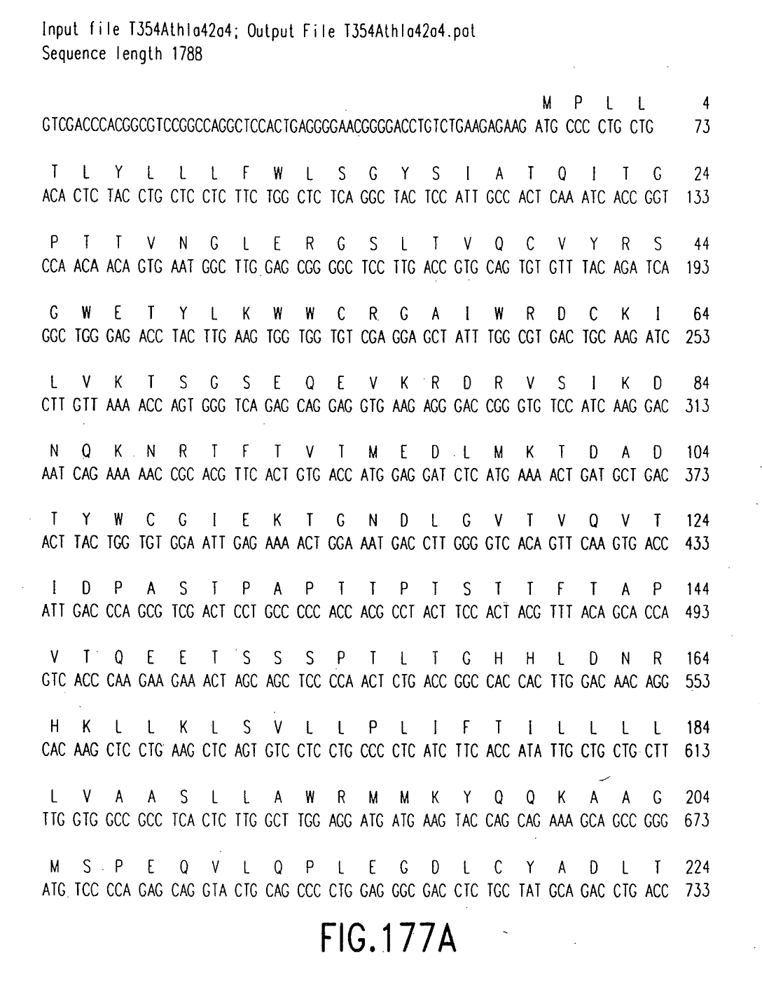

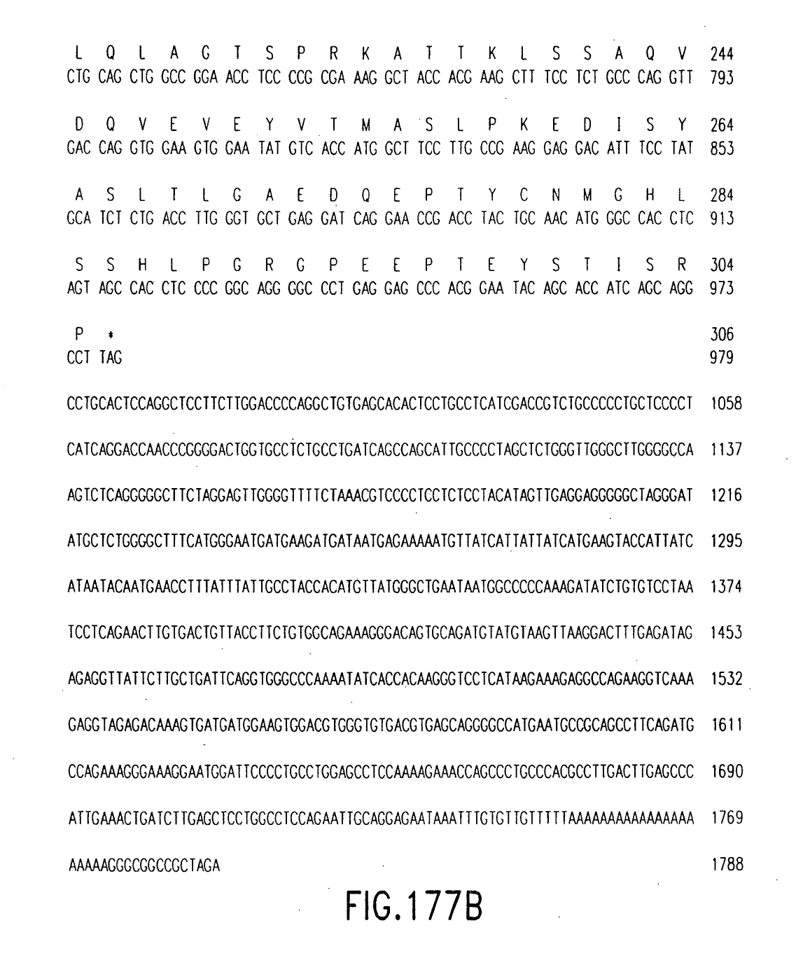

This analysis led to the identification of a clone, jthLa042a04, encoding full-length human TANGO 354. The cDNA of this clone is 1788 nucleotides long (FIG. 177A-177B; SEQ ID NO:119). The 915 nucleotide open reading frame of this cDNA (nucleotides 62-976 of SEQ ID NO:119) encodes a 305 amino acid protein (SEQ ID NO: 120).

Human TANGO 354 that has not been post-translationally modified is predicted to have a molecular weight of 33.8 kDa prior to cleavage of its signal peptide (31.6 kDa after cleavage of its signal peptide).

The signal peptide prediction program SIGNALP (Nielsen et al., 1997, Protein Engineering 10:1-6) predicted that human TANGO 354 includes a 19 amino acid signal peptide at amino acid 1 to about amino acid 19 preceding the mature human TANGO 354 protein which corresponds to about amino acid 20 to amino acid 305 of SEQ ID NO: 120.

Human TANGO 354 is a transmembrane protein having an extracellular domain which extends from about amino acid 20 to about amino acid 169, a transmembrane domain which extends from about amino acid 170 to about amino acid 193, and a cytoplasmic domain which extends from about amino acid 194 to amino acid 305 of SEQ ID NO: 120.

Alternatively, in another embodiment, a human TANGO 354 protein contains an extracellular domain which extends from about amino acid 194 to amino acid 305, a transmembrane domain which extends from about amino acid 170 to about amino acid 193, and a cytoplasmic domain which extends from about amino acid 20 to about amino acid 169 of SEQ ID NO: 120.

Human TANGO 354 includes an immunoglobulin domain at amino acids 33-110 of SEQ ID NO: 120. FIG. 179 depicts alignments of the immunoglobulin domains of TANGO 354 with consensus hidden Markov model immunoglobulin domains.

An N-glycosylation site is present at amino acids 88-91. A cAMP and cGMP-dependent protein kinase phosphorylation site is present at amino acids 233-236. Protein kinase C phosphorylation sites are present at amino acids 81-83, 231-233, and 236-238. Casein kinase II phosphorylation sites are present at amino acids 4447, 69-72, 81-84, 94-97, 101-104, 113-116, and 146-149. A tyrosine kinase phosphorylation site is present at amino acids 291-299. N-myristylation sites are present at amino acids 30-35, and 109-114.

Clone jthLa042a04, which encodes human TANGO 354, was deposited as EpT354 with the American Type Culture Collection (ATCC® 10801 University Boulevard, Manassas, Va. 20110-2209) on Jun. 18, 1999 and assigned Accession Number PTA-249. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

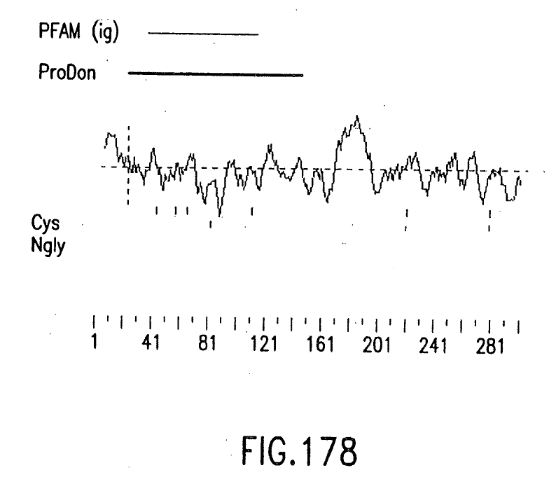

FIG. 178 depicts a hydropathy plot of human TANGO 354. The hydropathy plot indicates the presence of a hydrophobic domain within human TANGO 354, suggesting that human TANGO 354 is a transmembrane protein.

### Use of TANGO 354 Nucleic Acids, Polypeptides, and Modulators Thereof

TANGO 354 includes an immunoglobulin-like domain. Proteins having such domains play a role in mediating protein-protein and protein-ligand interactions, and thus can influence a wide variety of biological processes, including modulation of cell surface recognition; modulation of cellular motility, e.g., chemotaxis and chemokinesis; transduction of an extracellular signal (e.g., by interacting with a ligand and/or a cell-surface receptor); and/or modulation of a signal transduction pathways.

TANGO 354 polypeptides, nucleic acids, and modulators thereof can be used to modulate function, survival, morphology, migration, proliferation and/or differentiation of cells in the tissues in which it is expressed (e.g., hematopoietic tissues).

Because of the presence of an immunoglobulin domain and the expression of TANGO 354 in hematopoietic cells, TANGO 354 polypeptides, nucleic acids, and modulators thereof can be used to modulate (e.g., increase or decrease) hematopoietic function, thereby influencing one or more of: (1) regulation of hematopoiesis; (2) modulation of haemostasis; (3) modulation of an inflammatory response; (4) modulation of neoplastic growth, e.g., inhibition of tumor growth; and/or (5) regulation of thrombolysis.

Accordingly, TANGO 354 polypeptides, nucleic acids, and modulators thereof can be used to treat a variety of hematopoietic diseases including, but not limited to, myeloid disorders and/or lymphoid malignancies. Exemplary myeloid diseases that can be treated include acute promyeloid leukemia (APML), acute myelogenous leukemia (AML) and chronic myelogenous leukemia (CML) (reviewed in Vaickus, 1991, Crit. Rev. in Oncol./Hemotol. 11:267-97). Exemplary lymphoid malignancies that can be treated using these molecules include acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF) and Hodgkin's disease.

In one embodiment, TANGO 354 polypeptides, nucleic acids, and modulators thereof can be used to treat a variety of neoplastic diseases, including malignancies of the various organ systems, such as affecting lung, breast, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

TANGO 354 polypeptides, nucleic acids, and modulators thereof can also be used to treat a variety of non-cancerous diseases or conditions involving, for example, aberrant T cell activity (e.g., aberrant T cell proliferation and/or secretion). Examples of such T cell diseases or conditions include inflammation; allergy, for example, atopic allergy; organ rejection after transplantation (e.g., skin graft, cardiac graft, islet graft); graft-versus-host disease; autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosus, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis).

Further, in light of TANGO 345's presence in a Mixed Lymphocyte Reaction cDNA library, TANGO 345 expression can be utilized as a marker for specific tissues (e.g., lymphoid tissues such as the thymus and spleen) and/or cells (e.g., lymphocytes) in which TANGO 345 is expressed. TANGO 345 nucleic acids can also be utilized for chromosomal mapping.

### TANGO 378

A cDNA encoding human TANGO 378 was identified by analyzing the sequences of clones present in a human natural killer cell cDNA library.

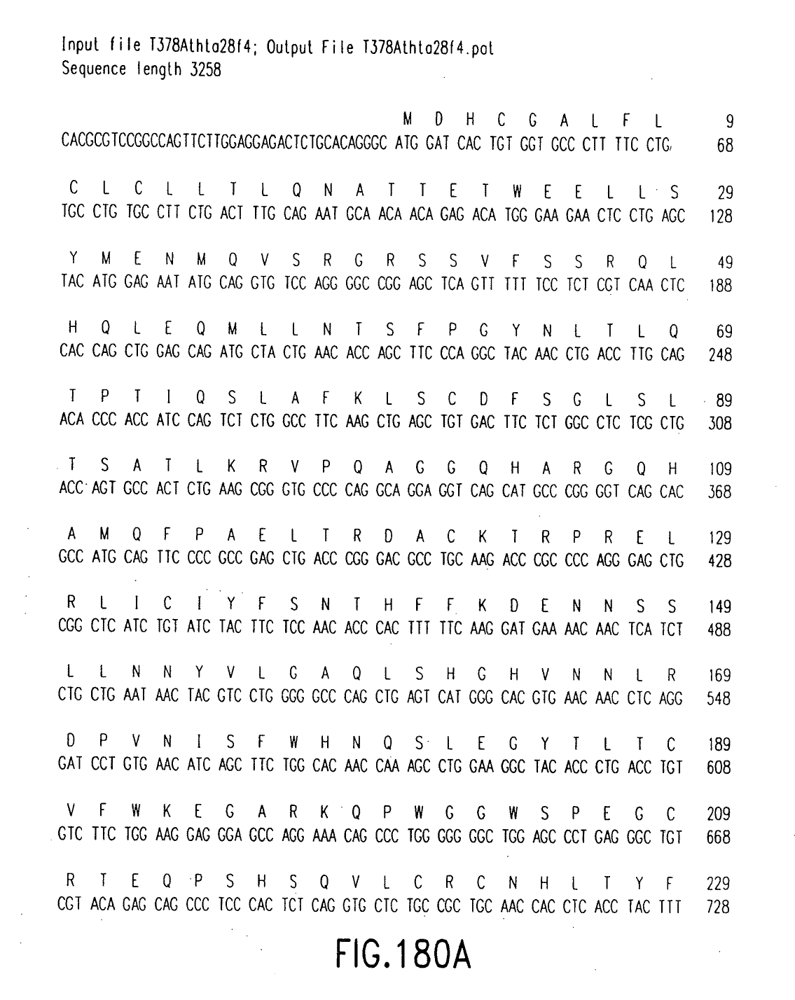

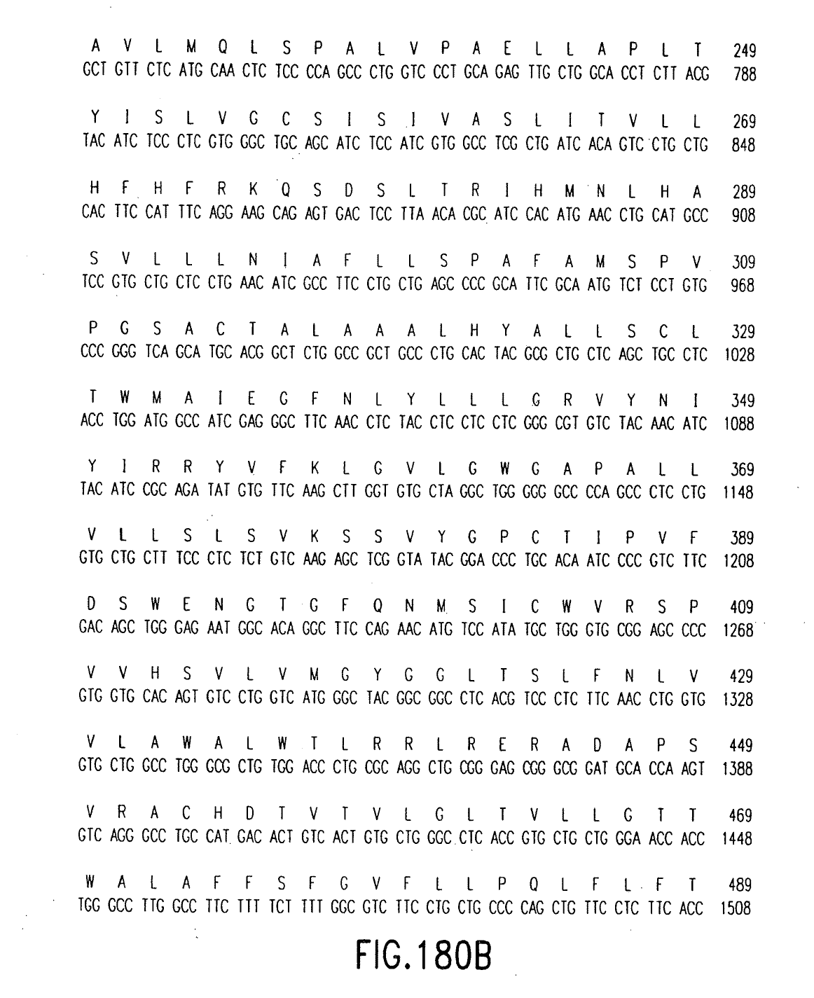

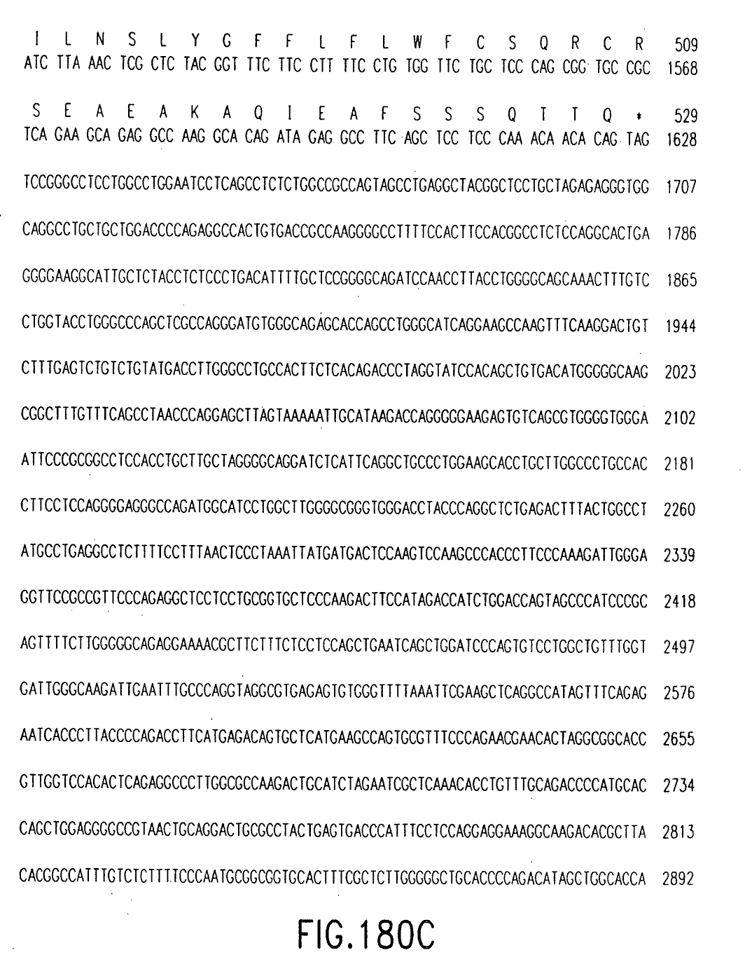

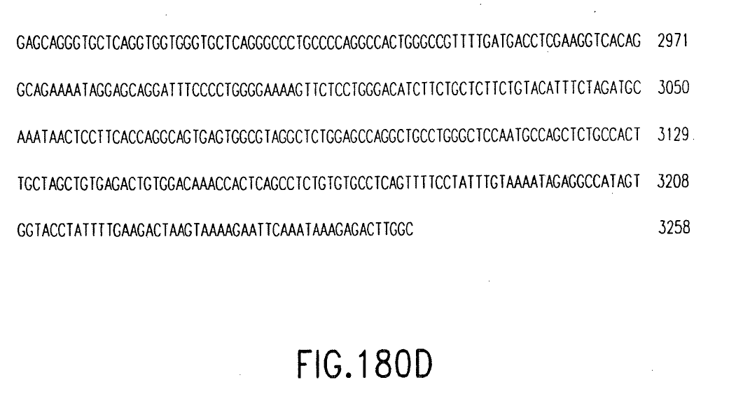

This analysis led to the identification of a clone, jthta028f04, encoding full-length human TANGO 378. The cDNA of this clone is 3258 nucleotides long (FIG. 180A-180D; SEQ ID NO: 123). The 1584 nucleotide open reading frame of this cDNA (nucleotides 42 to 1625 of SEQ ID NO:123) encodes a 528 amino acid protein (SEQ ID NO: 124).

The signal peptide prediction program SIGNALP (Nielsen et al., 1997, Protein Engineering 10:1-6) predicted that human TANGO 378 includes a 21 amino acid signal peptide at amino acid 1 to about amino acid 21 preceding the mature human MANGO 347 protein which corresponds to about amino acid 22 to amino acid 528 of SEQ ID NO:124.

Human TANGO 378 that has not been post-translationally modified is predicted to have a molecular weight of 59.0 kDa prior to cleavage of its signal peptide and a molecular weight of 56.7 kDa subsequent to cleavage of its signal peptide.

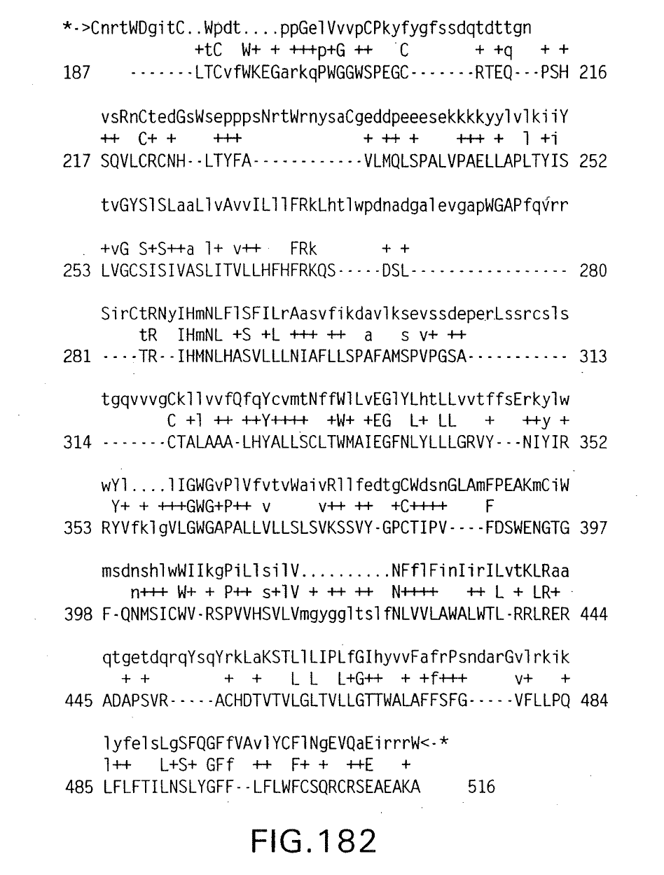

Human TANGO 378 is a seven transmembrane G-protein coupled receptor (GPCR) protein having an N-terminal extracellular domain which extends from about amino acid 22 to about amino acid 244; seven transmembrane domains which extend from about amino acids 245 to about amino acid 269, about amino acids 287 to about amino acid 306, about amino acids 323 to about amino acid 343, about amino acids 358 to about amino acid 376, about amino acids 414 to about amino acid 438, about amino acids 457 to about amino acid 477, and about amino acids 485 to about amino acid 504; and a C-terminal cytoplasmic domain which extends from about amino acid 505 to amino acid 528 of SEQ ID NO: 124. FIG. 182 depicts an alignment of each of the transmembrane domains of TANGO 378 with the consensus hidden Markov model seven transmembrane receptor sequences.

Alternatively, in another embodiment, a human TANGO 378 protein contains an N-terminal extracellular domain which extends from about amino acid 505 to amino acid 528; seven transmembrane domains which extend from about amino acids 245 to about amino acid 269, about amino acids 287 to about amino acid 306, about amino acids 323 to about amino acid 343, about amino acids 358 to about amino acid 376, about amino acids 414 to about amino acid 438, about amino acids 457 to about amino acid 477, and about amino acids 485 to about amino acid 504; and a C-terminal cytoplasmic domain which extends from about amino acid 22 to about amino acid 244 of SEQ ID NO: 124.

Human TANGO 378 includes three extracellular loops which extend from about amino acid 307 to about amino acid 322, about amino acid 377 to about amino acid 413, and about amino acid 478 to about amino acid 484 of SEQ ID NO:124.

Human TANGO 378 includes three intracellular loops which extend from about amino acid 270 to about amino acid 286, about amino acid 344 to about amino acid 357, and about amino acid 439 to about amino acid 456 of SEQ ID NO:124.

Based on structural similarities, TANGO 378 family members can be classified as members of the superfamily of G-protein coupled receptor. As used herein, the term “G protein-coupled receptor” or “GPCR” refers to a family of proteins that preferably comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the GPCR family also share certain conserved amino acid residues, some of which have been determined to be critical to receptor function and/or G protein signaling. An alignment of the transmembrane domains of 44 representative GPCRs can be found at http://mgdkk1.nidll.nih.gov:8000/extended.html.

Accordingly, in one embodiment, TANGO 378 family members can include at least one, two, three, four, five, six, or preferably, seven transmembrane domains, and thus has a “7 transmembrane receptor profile”. As used herein, the term “7 transmembrane receptor profile” includes an amino acid sequence having at least about 10-300, preferably about 15-200, more preferably about 20-100 amino acid residues, or at least about 22-100 amino acids in length and having a bit score for the alignment of the sequence to the 7tm_{—}1 family Hidden Markov Model (HMM) of at least 10, preferably 20-30, more preferably 22-40, more preferably 40-50, 50-75, 75-100, 100-200 or greater. The 7tm_{—}1 family HMM has been assigned the PFAM Accession PF00001 (http://genome.wustl.edu/Pfan/WWWdata/7tm_{—}1.html). In one embodiment, the seven transmembrane domains of TANGO 378 extend from about amino acids 245 to about amino acid 269, about amino acids 287 to about amino acid 306, about amino acids 323 to about amino acid 343, about amino acids 358 to about amino acid 376, about amino acids 414 to about amino acid 438, about amino acids 457 to about amino acid 477, and about amino acids 485 to about amino acid 504; and a C-terminal cytoplasmic domain which extends from about amino acid 505 to amino acid 528 of SEQ ID NO: 122. FIG. 182 depicts an alignment of each of the transmembrane domains of TANGO 378 with the consensus hidden Markov model seven transmembrane receptor domain of SEQ ID NO: 122.

To identify the presence of a 7 transmembrane receptor profile in a TANGO 378, the amino acid sequence of the protein is searched against a database of HMMs (e.g., the Pfam database, release 2.1) using the default parameters (http://www.sanger.ac.uk/Software/Pfam/HMM_search). For example, the hmmsf program, which is available as part of the HMMER package of search programs, is a family specific default program for PF00001 and score of 15 is the default threshold score for determining a hit. Alternatively, the seven transmembrane domain can be predicted based on stretches of hydrophobic amino acids forming a-helices (SOUSI server). Accordingly, proteins having at least 50-60% identity, preferably about 60-70%, more preferably about 70-80%, or about 80-90% identity with the 7 transmembrane receptor profile of human TANGO 378 are within the scope of the invention.

TANGO 378 family members can include at least one, preferably two, and most preferably three extracellular loops. As defined herein, the term “loop” includes an amino acid sequence having a length of at least about 4, preferably about 5-10, preferably about 10-20, and more preferably about 20-30, 30-40, 40-50, 50-60, 60-70, 70-80, 80-90, 90-100, or 100-150 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring TANGO 378 or TANGO 378-like molecule, and the C-terminal amino acid of a loop is adjacent to an N-terminal amino acid of a transmembrane domain in a naturally-occurring TANGO 378 or TANGO 378-like molecule. Examples of TANGO 378 extracellular loops can be found at about amino acids 307-322, 377-413, and 478-484 of SEQ ID NO: 122.

TANGO 378 family members can include at least one, preferably two, and most preferably three cytoplasmic loops. Examples of TANGO 378 cytoplasmic loops are found at about amino acids 270-286, 344-357, and 439-456 of the polypeptide of SEQ ID NO: 122.

In one embodiment, a TANGO 378 family member includes a 7 transmembrane receptor profile and three extracellular loops. In another embodiment, a TANGO 378 family member includes a 7 transmembrane receptor profile, three extracellular loops, and three cytoplasmic loops. In yet another embodiment, a TANGO 378 family member includes a 7 transmembrane receptor profile, three extracellular loops, three cytoplasmic loops, and an extracellular N-terminal domain. In another embodiment, a TANGO 378 family member includes a 7 transmembrane receptor profile, three extracellular loops, three cytoplasmic loops, an extracellular N-terminal domain, and a C-terminal cytoplasmic domain. In another embodiment, a TANGO 378 family member includes a 7 transmembrane receptor profile, three extracellular loops, three cytoplasmic loops, an extracellular N-terminal domain, a C-terminal cytoplasmic domain, and a signal peptide.

N-glycosylation sites are present at amino acids 18-21, 58-61, 65-68, 146-149, 173-176, 179-182, 394-397, and 400-403. A cAMP and cGMP-dependent protein kinase phosphorylation site is present at amino acids 274-277. Protein kinase C phosphorylation sites are present at amino acids 45-47, 93-95, 375-377, 437-439, 449-451, and 505-507. Casein kinase II phosphorylation sites are present at amino acids 23-26, 29-32, and 510-513. N-myristylation sites are present at amino acids 86-91, 101-106, 157-162, 255-260, 311-316, 420-425, and 467-472. A thiol (cysteine) protease histidine site is present at amino acid 410-420.

Clone jthta028f04, which encodes human TANGO 378, was deposited as EpT378 with the American Type Culture Collection (ATCC® 10801 University Boulevard, Manassas, Va. 20110-2209) on Jun. 18, 1999 and assigned Accession Number PTA-249. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

FIG. 182 depicts a hydropathy plot of human TANGO 378. The hydropathy plot indicates that human TANGO 378 has a signal peptide at its amino terminus and seven hydrophobic domains within human TANGO 378, suggesting that human TANGO 378 is a transmembrane protein.

### Use of TANGO 378 Nucleic Acids, Polypeptides, and Modulators Thereof

TANGO 378 includes a seven transmembrane domain which is typically found in G-protein coupled receptors. Proteins having such a domain play a role in transducing an extracellular signal, e.g., by interacting with a ligand and/or a cell-surface receptor, followed by mobilization of intracellular molecules that participate in signal transduction pathways (e.g., adenylate cyclase, or phosphatidylinositol 4,5-bisphosphate (PIP₂), inositol 1,4,5-triphosphate (IP₃)).

TANGO 378 polypeptides, nucleic acids, and modulators thereof can be used to modulate function, survival morphology, migration, proliferation and/or differentiation of ells in the tissues in which it is expressed (e.g., natural killer cells). For example, TANGO 354 polypeptides, nucleic acids, and modulators thereof can be used to modulate an immune response in a subject by, for example, (1) modulating immune cytotoxic responses against pathogenic organisms, e.g., viruses, bacteria, and parasites; (2) by modulating organ rejection after transplantation (e.g., skin graft, cardiac graft, islet graft); (3) by modulating immune recognition and lysis of normal and malignant cells; (4) by modulating T cell diseases; and (5) by controlling neoplastic growth, e.g., inhibition of tumor growth.

Accordingly, TANGO 378 polypeptides, nucleic acids, and modulators thereof can be used to treat a variety of diseases involving aberrant immune responses, for example, aberrant T cell activity (e.g., aberrant T cell proliferation and/or secretion). A non-limiting list of diseases involving aberrant T cell activity is provided in the section entitled “TANGO 354” above.

In other embodiments, TANGO 378 polypeptides, nucleic acids, and modulators thereof can be used to treat a variety of neoplastic diseases, including hematopoietic malignancies and including, but not limited to, myeloid disorders, lymphoid malignancies, and/or malignancies of the various organ systems. A non-limiting list of such neoplastic diseases is provided in the section entitled “TANGO 354” above.

Further, in light of TANGO 378's presence in a Natral Killer cell cDNA library, TANGO 378 expression can be utilized as a marker for specific tissues (e.g., lymphoid tissues such as the thymus and spleen) and/or cells (e.g., Natural Killer cells) in which TANGO 345 is expressed. TANGO 345 nucleic acids can also be utilized for chromosomal mapping.

The TANGO 339, TANGO 358, TANGO 365, TANGO 368, TANGO 369, TANGO 383, MANGO 346 and MANGO 349 proteins and nucleic acid molecules comprise families of molecules having certain conserved structural and functional features.

For example, TANGO 339 proteins, TANGO 358 proteins, TANGO 365 proteins, TANGO 368 proteins, TANGO 369 proteins, TANGO 383 proteins, MANGO 346 proteins and MANGO 349 proteins of the invention can have signal sequences. Thus, in one embodiment, a TANGO 339 protein contains a signal sequence of about amino acids 1 to 42.

In another embodiment, a TANGO 358 protein contains a signal sequence at about amino acids 1 to 42. In another embodiment, a TANGO 365 protein contains a signal sequence of about amino acids 1 to 36. In another embodiment, a TANGO 368 protein contains a signal sequence of about amino acids 1 to 27. In another embodiment, a TANGO 369 protein contains a signal sequence of about amino acids 1 to 26. In another embodiment, a TANGO 383 protein contains a signal sequence of about amino acids 1 to 20. In another embodiment, a MANGO 346 protein contains a signal sequence of about amino acids 1 to 19. In another embodiment, a MANGO 349 protein contains a signal sequence of about amino acids 1 to 26. The signal sequence is usually cleaved during processing of the mature protein. In the case of, e.g., transmembrane 4-type proteins, the signal peptide is generally not cleaved, but becomes a transmembrane-anchoring domain of the polypeptide.

A TANGO 339 family member can include one or more of the following domains: (1) an extracellular domain; (2) a transmembrane domain; and (3) a cytoplasmic domain. In one embodiment, a TANGO 339 protein contains extracellular domains at about amino acid residues 43 to 61 and 116 to 232, transmembrane domains at about amino acid residues 62 to 84, 93 to 115, and 233 to 254, and cytoplasmic domains at about amino acid residues 85 to 92 and 255 to 270. In this embodiment, the mature TANGO 339 protein corresponds to amino acids 43 to 270.

In another embodiment, a TANGO 339 protein contains extracellular domains at about amino acid residues 1 to 16, 85 to 92, and 255 to 270, transmembrane domains at about amino acid residues 17 to 41, 62 to 84, 93 to 115, and 233 to 254, and cytoplasmic domains at about amino acid residues 42 to 61 and 116 to 232. In this embodiment, the mature TANGO 339 protein corresponds to amino acids 1 to 270.

A TANGO 339 family member can include a signal sequence. In certain embodiment, a TANGO 339 family member has the amino acid sequence, and the signal sequence is located at amino acids 1 to 40, 1 to 41, 1 to 42, 1 to 43 or 1 to 44. In such embodiments of the invention, the domains and the mature protein resulting from cleavage of such signal peptides are also included herein. For example, the cleavage of a signal sequence consisting of amino acids 1 to 40 results in an extracellular domain consisting of amino acids 41 to 61 and the mature TANGO 339 protein corresponding to amino 41 to 270.

A TANGO 339 family member can include one or more transmembrane 4 or transmembrane 4-like domains. A transmembrane 4 domain typically has the following consensus sequence: G-xxx-[LIVMF]-xx-[GSA]-[LIVMF][LIVMF]-G-C-x-[GA]-[STA]-xx-[EG]-xx-[CWN]-[LIVM][LIVM], wherein G is a glycine residue, “x” is any amino acid, [LIVMF] is a leucine, isoleucine, valine, methionine or phenylalanine residue, [GA] is either a glycine or an alanine residue, [STA] is a serine, threonine or alanine residue, [EG] is either a glutamic acid or glycine residue, [CWN] is cysteine, tryptophan or asparagine residue. A transmembrane 4 domain is a characteristic of transmembrane 4 superfamily members which include, for example, CD9 antigen, CD37, CD53, CD63, CD81, and CD82. Transmembrane 4 proteins have the following characteristics: they are type III membrane proteins, which contain an N-terminal membrane-anchoring domain that is not cleaved during biosynthesis and that functions both as a translocation signal and as a membrane anchor; they contain a total of four transmembrane domains and at least seven conserved cysteine residues; and they are approximately 218 to 284 amino acid residues.

A transmembrane 4-like domain as described herein can have the following consensus sequence: G-xxx-[LIVMF]-xx-[GSA]-[LIVM]-x-G-C-x-[GA]-[STA]-xx-[EG]-xx-[CWN]-[LIVM][LIVM], wherein G is a glycine residue, “x” is any amino acid, [LIVMF] is a leucine, isoleucine, valine, methionine or phenylalanine residue, [GA] is either a glycine or an alanine residue, [STA] is a serine, threonine or alanine residue, [EG] is either a glutamic acid or glycine residue, [CWN] is cysteine, tryptophan or asparagine residue.

In one embodiment, a TANGO 339 family member has the amino acid sequence and, preferably, a transmembrane 4 domain-like consensus sequence is located at about amino acid positions 69 to 91. In another embodiment, a TANGO 339 family member has the amino acid sequence and, preferably, a transmembrane 4-like domain is located at about amino acid positions 68 to 260. In another embodiment, a TANGO 339 family member includes one or more transmembrane 4-like domain consensus sequences having an amino acid sequence that is at least about 55%, preferably at least about 65%, more preferably at least 75%, yet more preferably at least about 85%, and most preferably at least about 95% identical to amino acids 69 to 91. In yet another embodiment, a TANGO 339 family member includes one or more transmembrane 4-like domains having an amino acid sequence that is at least about 55%, preferably at least about 65%, more preferably at least 75%, yet more preferably at least about 85%, and most preferably at least about 95% identical to amino acids 68 to 261.

In another embodiment, a TANGO 339 family member includes one or more transmembrane 4-like domain consensus sequences having an amino acid sequence that is at least about 55%, preferably at least about 65%, more preferably at least 75%, yet more preferably at least about 85%, and most preferably at least about 95% identical to amino acids 69 to 91, and has at least one TANGO 339 biological activity as described herein. In yet another embodiment a TANGO 339 family member includes one or more transmembrane 4-like domains having an amino acid sequence that is at least about 55%, preferably at least about 65%, more preferably at least 75%, yet more preferably at least about 85%, and most preferably at least about 95% identical to amino acids 68 to 261, and has at least one TANGO 339 biological activity as described herein.

In another embodiment, the transmembrane 4-like domain of TANGO 339 is a transmembrane 4 domain, which has the following consensus sequence: G-xxx-[LIVMF]-xx-[GSA]-[LIVMF][LWIF]-G-C-x-[GA]-[STA]-xx-[EG]-xx-[CWN]-[LIVM][LWM], wherein G is a glycine residue, “x” is any amino acid, [LIVMF] is a leucine, isoleucine, valine, methionine or phenylalanine residue, [GA] is either a glycine or an alanine residue, [STA] is a serine, threonine or alanine residue, [EG] is either a glutamic acid or glycine residue, [CWN] is cysteine, tryptophan or asparagine residue. In this embodiment, a TANGO 339 family member includes one or more transmembrane alike domains having an amino acid sequence that is at least about 55%, preferably at least about 65%, more preferably at least 75%, yet more preferably at least about 85%, and most preferably at least about 95% identical to amino acids 68 to 261.

In another embodiment, a TANGO 339 family member includes one or more peripherin/rom-1 or peripherin/rom-1-like domains. A peripherin/rom-1 domain typically has the following consensus sequence: D-G-V-P-F-S-C-C-N-P-x-S-P-R-P-C, wherein D is an aspartic acid residue, G is a glycine residue, V is a valine residue, P is a proline residue, F is a phenylalanine residue, S is a serine residue, C is a cysteine residue, N is an asparagine residue, x is any amino acid, and R is an arginine residue. Peripherin/rom-1 domains are characteristic of retinal-specific integral membrane proteins that are located at the rims of the photoreceptor disks and that function in disk morphogenesis. Peripherin (or RDS) and rom-1 are examples of proteins that contain the peripherin/rom-1 domain. Defects in the peripherin gene have been shown to cause various diseases, including autosomal dominant retinitis pigmentosa, autosomal dominant punctata albescens, and butterfly-shaped pigment dystrophy.

A peripherin/rom-1-like domain as described herein has the following consensus sequence: G-V-P-F-S-C-C-x-P, wherein G is a glycine residue, V is a valine residue, P is a proline residue, F is a phenylalanine residue, and C is a cysteine residue. In one embodiment, a TANGO 339 family member has the amino acid sequence and, preferably, a peripherin/rom-1-like domain consensus sequence is located at about amino acid positions 181 to 189. In another embodiment, a TANGO 339 family member has the amino acid sequence of, preferably, a peripherin/rom-1-like domain is located at about amino acid positions 18 to 270.

In another embodiment, a TANGO 339 family member includes one or more peripherin/rom-1-like domain consensus sequences having an amino acid sequence that is at least about 55%, preferably at least about 65%, more preferably at least 75%, yet more preferably at least about 85%, and most preferably at least about 95% identical to amino acids 181 to 189. In another embodiment, a TANGO 339 family member includes one or more peripherin/rom-1-like domain having an amino acid sequence that is at least about 55%, preferably at least about 65%, more preferably at least 75%, yet more preferably at least about 85%, and most preferably at least about 95% identical to amino acid positions 18 to 270.

In another embodiment, a TANGO 339 family member includes one or more peripherin/rom-1-like domain consensus sequences having an amino acid sequence that is at least about 55%, preferably at least about 65%, more preferably at least 75%, yet more preferably at least about 85%, and most preferably at least about 95% identical to amino acids 181 to 189, and has at least one TANGO 339 biological activity as described herein. In yet another embodiment, a TANGO 339 family member includes one or more peripherin/rom-1-like domain having an amino acid sequence that is at least about 55%, preferably at least about 65%, more preferably at least 75%, yet more preferably at least about 85%, and most preferably at least about 95% identical to amino acid positions 18 to 270, and has at least one TANGO 339 biological activity as described herein.

In another embodiment, the peripherin/rom-1-like domain of TANGO 339 is a peripherin/rom-1 domain, which has the following consensus sequence: D-G-V-P-F-S-C-C-N-P-x-S-P-R-P-C, wherein D is an aspartic acid residue, G is a glycine residue, V is a valine residue, P is a proline residue, F is a phenylalanine residue, S is a serine residue, C is a cysteine residue, N is an asparagine residue, x is any amino acid, and R is an arginine residue. In this embodiment, a TANGO 339 family member includes one or more peripherin/rom-1-like domains having an amino acid sequence that is at least about 55%, preferably at least about 65%, more preferably at least 75%, yet more preferably at least about 85%, and most preferably at least about 95% identical to amino acids 18 to 270.

A TANGO 358 family member can include one or more of the following domains: (1) an extracellular domain; (2) a transmembrane domain; and (3) a cytoplasmic domain. In one embodiment, a TANGO 358 protein contains an extracellular domain at amino acids 1 to about 49 or a mature extracellular domain at about amino acid residues 43 to 49, a transmembrane domain at about amino acid residues 50 to 66, and a cytoplasmic domain at about amino acid residues 67 to 82.

A TANGO 358 family member can include a signal sequence. In certain embodiment, a TANGO 358 family member has the amino acid sequence, and the signal sequence is located at amino acids 1 to 40, 1 to 41, 1 to 42, 1 to 43 or 1 to 44. In such embodiments of the invention, the mature protein resulting from cleavage of such signal peptides are also included herein. For example, the cleavage of a signal sequence consisting of amino acids 1 to 40 results in an extracellular domain consisting of amino acids residues 41 to 50 and the mature TANGO 368 protein corresponding to amino acid residues 41 to 82.

A TANGO 365 family member can include a signal sequence. In certain embodiments, a TANGO 365 family member has the amino acid sequence of SEQ ID NO:130, and the signal sequence is located at amino acids 1 to 34, 1 to 35, 1 to 36, 1 to 37 or 1 to 38. In such embodiments of the invention, the extracellular domain and the mature protein resulting from cleavage of such signal peptides are also included herein. For example, the cleavage of a signal sequence consisting of amino acids 1 to 36 results in a mature TANGO 365 protein corresponding to amino 37 to 165.

A TANGO 365 family member can include one or more of the following domains: (1) an extracellular domain; (2) two transmembrane domains; and (3) a cytoplasmic domain. Thus, in one embodiment, a TANGO 365 protein contains an extracellular domain of about amino acids 95 to 165, or a mature extracellular domain of about amino acids 30 to 246. In another embodiment, a TANGO 365 protein contains a first transmembrane domain of about amino acids 52 to 70. In another embodiment, an protein contains a cytoplasmic domain of about amino acids 71 to 77. In another embodiment, a TANGO 365 protein contains a second transmembrane domain of about amino acids 78 to 94. In yet another embodiment, a TANGO 365 protein is a mature protein containing an extracellular domain, two transmembrane domains and a cytoplasmic domain of about amino acids 37 to 165.

A TANGO 368 family member can include a signal sequence. In certain embodiments, a TANGO 368 family member has the amino acid sequence of SEQ ID NO:132, and the signal sequence is located at amino acids 1 to 25, 1 to 26, 1 to 27, 1 to 28 or 1 to 29. In such embodiments of the invention, the mature protein resulting from cleavage of such signal peptides are also included herein. For example, the cleavage of a signal sequence consisting of amino acids 1 to 27 results in a mature TANGO 368 protein corresponding to amino 28 to 59.

A TANGO 369 family member can include a signal sequence. In certain embodiments, a TANGO 369 family member has the amino acid sequence of SEQ ID NO:134, and the signal sequence is located at amino acids 1 to 24, 1 to 25, 1 to 26, 1 to 27 or 1 to 28. In such embodiments of the invention, the mature protein resulting from cleavage of such signal peptides are also included herein. For example, the cleavage of a signal sequence consisting of amino acids 1 to 26 results in a mature TANGO 368 protein corresponding to amino 27 to 58.

A TANGO 383 family member can include a signal sequence. In certain embodiments, a TANGO 383 family member has the amino acid sequence of SEQ ID NO:136, and the signal sequence is located at amino acids 1 to 18, 1 to 19, 1 to 20, or 1 to 21. In such embodiments of the invention, the extracellular domain and the mature protein resulting from cleavage of such signal peptides are also included herein. For example, the cleavage of a signal sequence consisting of amino acids 1 to 20 results in a mature TANGO 383 protein corresponding to amino 21 to 140.

A TANGO 383 family member can include one or more of the following domains: (1) an extracellular domain; (2) two transmembrane domains; and (3) a cytoplasmic domain.

In one embodiment, a TANGO 383 protein contains a cytoplasmic domain of about amino acids 21 to 49. In another embodiment, a TANGO 383 protein contains a first transmembrane domain of about amino acids 50 to 70. In another embodiment, a TANGO 383 protein contains an extracellular domain of about amino acids 71 to 115. In another embodiment, a TANGO 383 protein contains a second transmembrane domain of about amino acids 116 to 133. In yet another embodiment, a TANGO 383 protein is a mature protein containing an extracellular domain, two transmembrane domains and a cytoplasmic domain of about amino acids 21 to 140.

A MANGO 346 family member can include a signal sequence. In certain embodiments, a MANGO 346 family member has the amino acid sequence of SEQ ID NO:138, and the signal sequence is located at amino acids 1 to 17, 1 to 18, 1 to 19, 1 to 20 or 1 to 21. In such embodiments of the invention, the extracellular domain and the mature protein resulting from cleavage of such signal peptides are also included herein. For example, the cleavage of a signal sequence consisting of amino acids 1 to 19 results in the mature MANGO 346 protein corresponding to amino 20 to 60.

A MANGO 349 family member can include a signal sequence. In certain embodiments, a MANGO 349 family member has the amino acid sequence of SEQ ID NO:140, and the signal sequence is located at amino acids 1 to 24, 1 to 25, 1 to 26, 1 to 27 or 1 to 28. In such embodiments of the invention, the extracellular domain and the mature protein resulting from cleavage of such signal peptides are also included herein. For example, the cleavage of a signal sequence consisting of amino acids 1 to 26 results in the mature MANGO 349 protein corresponding to amino 27 to 167.

### Human TANGO 339

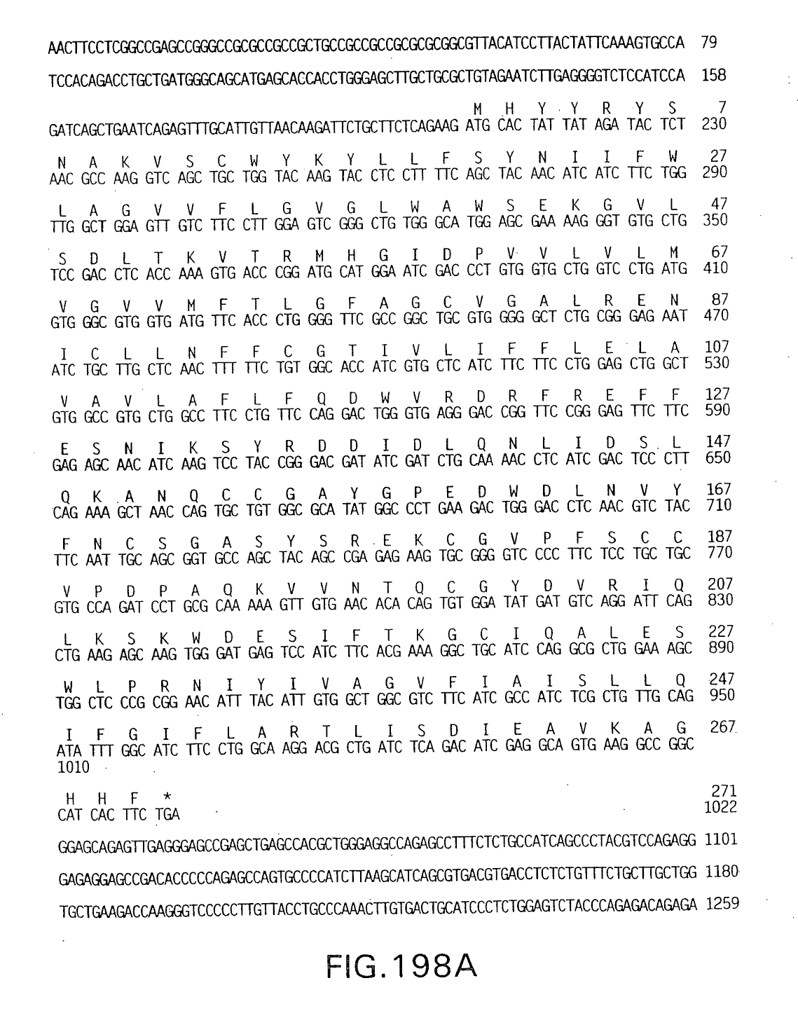

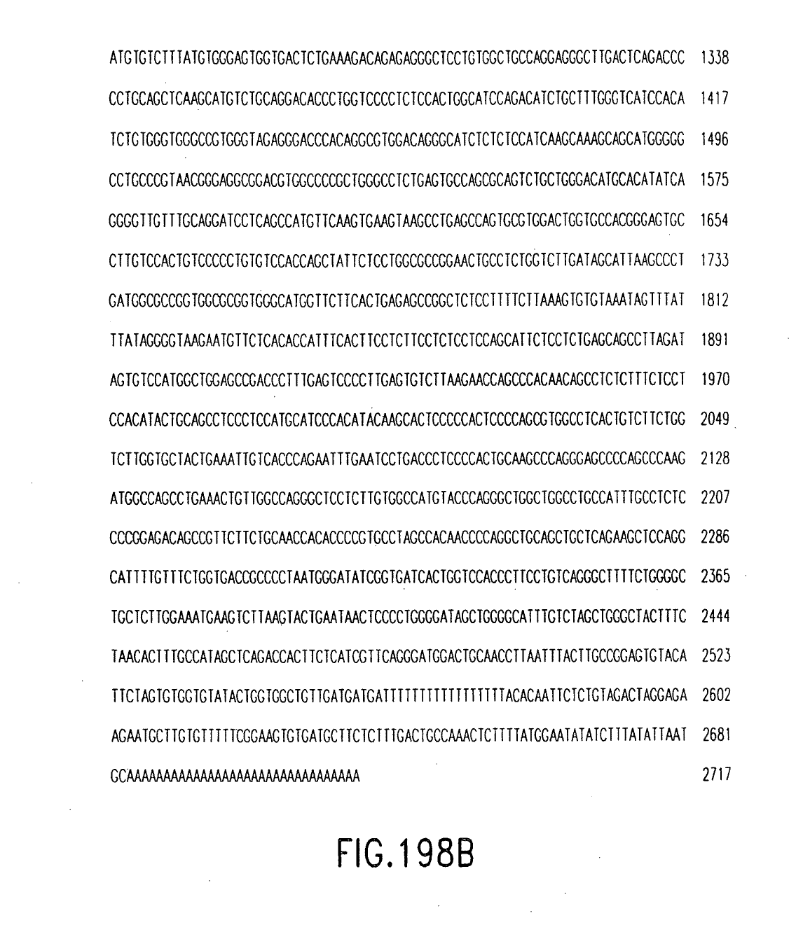

A cDNA encoding human TANGO 339 was identified by analyzing the sequences of clones present in a human fetal library for sequences that encode wholly secreted or transmembrane proteins. This analysis led to the identification of a clone, jthga100g01, encoding full-length human TANGO 339. The human TANGO 339 cDNA of this clone is 2715 nucleotides long (FIG. 198A-198B; SEQ ID NO:125). The open reading frame of this cDNA (nucleotides 210 to 1019 of SEQ ID NO: 125) encodes a 270 amino acid transmembrane protein (SEQ ID NO: 126).

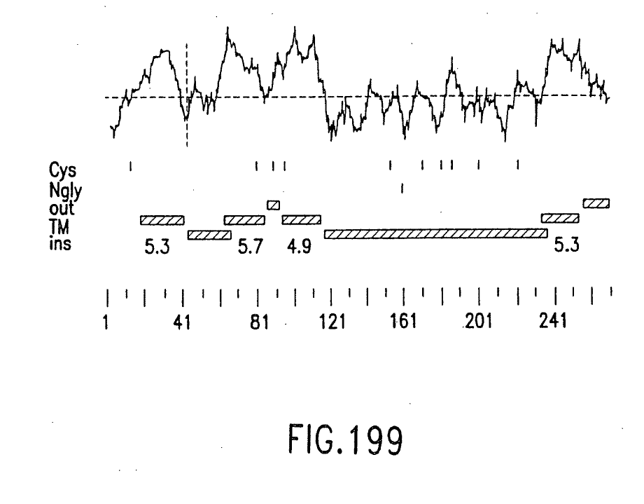

FIG. 199 depicts a hydropathy plot of human TANGO 339.

The signal peptide prediction program SIGNALP (Nielsen et al., 1997, Protein Engineering 10:1-6) predicted that human TANGO 339 includes a 42 amino acid signal peptide (amino acid 1 to amino acid 42) preceding the mature human TANGO 339 protein (corresponding to amino acid 43 to amino acid 270). In instances wherein the signal peptide is cleaved, the molecular weight of human TANGO 339 protein without post-translational modifications is 30.7 kDa prior to the cleavage of the signal peptide, and 25.6 kDa after cleavage of the signal peptide. The presence of a methionine residue at positions 56, 67 and 72 indicates that there can be alternative forms of human TANGO 339 of 215 amino acids, 204 amino acids, and 199 amino acids, respectively.

Human TANGO 339 protein is a transmembrane protein that contains extracellular domains at amino acid residues 43 to 61 and 116 to 232, transmembrane domains at amino acid residues 62 to 84, 93 to 115, and 233 to 254, and cytoplasmic domains at amino acid residues 85 to 92 and 255 to 270 of SEQ ID NO: 126.

In instances wherein the signal peptide is not cleaved, human TANGO 339 has extracellular domains at amino acid residues 1 to 16, 85 to 92, and 255 to 270, transmembrane domains at amino acid residues 17 to 41, 62 to 84, 93 to 115, and 233 to 254, and cytoplasmic domains of amino acid residues 42 to 61 and 116 to 232 of SEQ ID NO:126.

Alternatively, in another embodiment, a human TANGO 339 protein contains cytoplasmic domains at amino acid residues 43 to 61 and 116 to 232, transmembrane domains at amino acid residues 62 to 84, 93 to 115, and 233 to 254, and extracellular domains at amino acid residues 85 to 92 and 255 to 270.

In one embodiment of a nucleotide sequence of human TANGO 339, the nucleotide at position 29 is adenine (A). In this embodiment, the amino acid at position 10 is lysine (K). In an alternative embodiment, a species variant of human TANGO 339 has a nucleotide at position 29 which is guanine (G). In this embodiment, the amino acid at position 10 is arginine (R), i.e., a conservative substitution.

In another embodiment of a nucleotide sequence of human TANGO 339, the nucleotide at position 59 is thymine (T). In this embodiment, the amino acid at position 20 is phenylalanine (F). In an alternative embodiment, a species variant of human TANGO 339 has a nucleotide at position 59 which is adenine (A). In this embodiment, the amino acid at position 20 is tyrosine (Y), i.e., a conservative substitution.

In another embodiment of a nucleotide sequence of human TANGO 339, the nucleotide at position 119 is cytosine (C). In this embodiment, the amino acid at position 40 is alanine (A). In an alternative embodiment, a species variant of human TANGO 339 has a nucleotide at position 119 which is thymine (T). In this embodiment, the amino acid at position 40 is valine (V), i.e., a conservative substitution.

In another embodiment of a nucleotide sequence of human TANGO 339, the nucleotide at position 180 is cytosine (C). In this embodiment, the amino acid at position 60 is aspartate (D). In an alternative embodiment, a species variant of human TANGO 339 has a nucleotide at position 180 which is guanine (G). In this embodiment, the amino acid at position 60 is glutamate (E), i.e., a conservative substitution.

Human TANGO 339 includes a transmembrane 4-like domain (at amino acids 68 to 260 of SEQ ID NO: 126) and a peripherin/rom-1-like domain (at amino acids 18 to 270 of SEQ ID NO:126).

Human TANGO 339 has an N-glycosylation site with the sequence NCSG (at amino acid residues 169 to 172). Two protein kinase C phosphorylation sites are present in human TANGO 339. The first has the sequence SEK (at amino acid residues 42 to 44) and the second has the sequence SYR (at amino acid residues 133 to 135). Human TANGO 339 has three casein kinase II phosphorylation sites. The first has the sequence SYRD (at amino acid residues 133 to 136), the second has the sequence SKWD (at amino acid residues 210 to 213), and the third has the sequence SDIE (at amino acid residues 259 to 262). Six N-myristylation sites are present in human TANGO 339. The first has the sequence GCVGAL (at amino acid residues 79 to 84), the second has the sequence GASYSR (at amino acid residues 172 to 177), the third has the sequence GVPFSC (at amino acid residues 181 to 186), the fourth has the sequence GCIQAL (at amino acid residues 220 to 225), the fifth has the sequence GVFIAI (at amino acid residues 238 to 243), and the sixth has the sequence GIFLAR (at amino acid residues 250 to 255). Human TANGO 339 has a prokaryotic membrane lipoprotein lipid attachment site with the sequence VVMFTLGFAGC (at amino acid residues 70 to 80).

The human TANGO 339 gene maps to human chromosome 10 between markers D10S201 and D10S551. As retinal G protein coupled receptor and pulmonary-associated protein A1 map to this region of chromosome 10, TANGO 339 nucleic acids, proteins and modulators thereof can be used to diagnose disorders associated with G protein coupled receptors and/or modulate G protein coupled receptor-related processes, e.g. retinal processes and/or pulmonary-related processes.

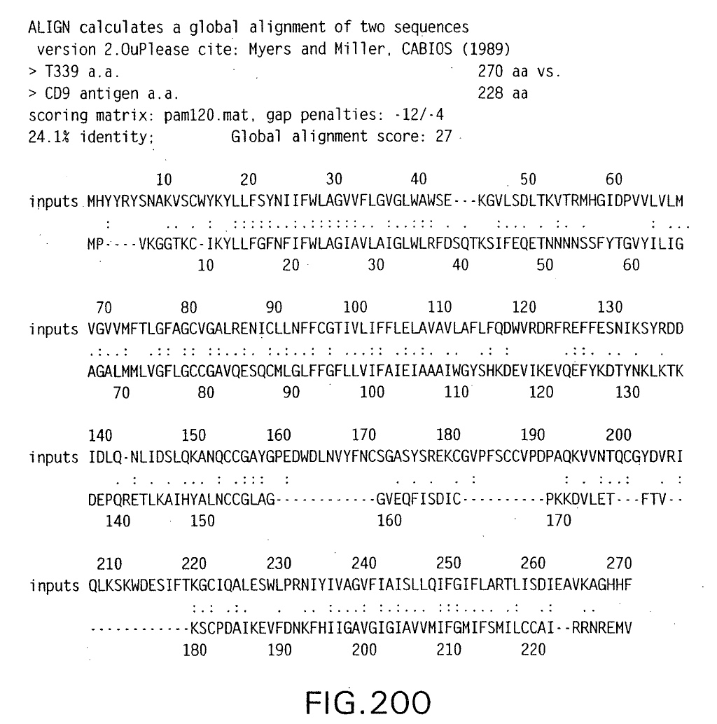

FIG. 200 shows an alignment of the human TANGO 339 amino acid sequence with the human CD9 antigen amino acid sequence (Accession Number NM_{—}001769). The alignment shows that there is a 24.1% overall amino acid sequence identity between human TANGO 339 and human CD9 antigen. The CD9 antigen is a widely expressed cell surface glycoprotein that has been shown to be involved in such processes as cell activation, proliferation, and adhesion. For example, CD9 antigen expression on platelets mediates platelet activation and aggregation. CD9 antigen has also been shown to be expressed by neural cells and can play a role in intercellular signaling in the nervous system, in particular, controlling cellular attraction or repulsion in guiding neural growth to target points. Further, the CD9 antigen has been shown to associate with beta 1 integrins and other transmembrane 4 superfamily members, including CD81 and CD82. As such TANGO 339 proteins, nucleic acids and modulators thereof could be useful in modulating cellular interaction such as between immune cells, and also can be involved in modulating intercellular signaling, such as neural cell intercellular signaling.

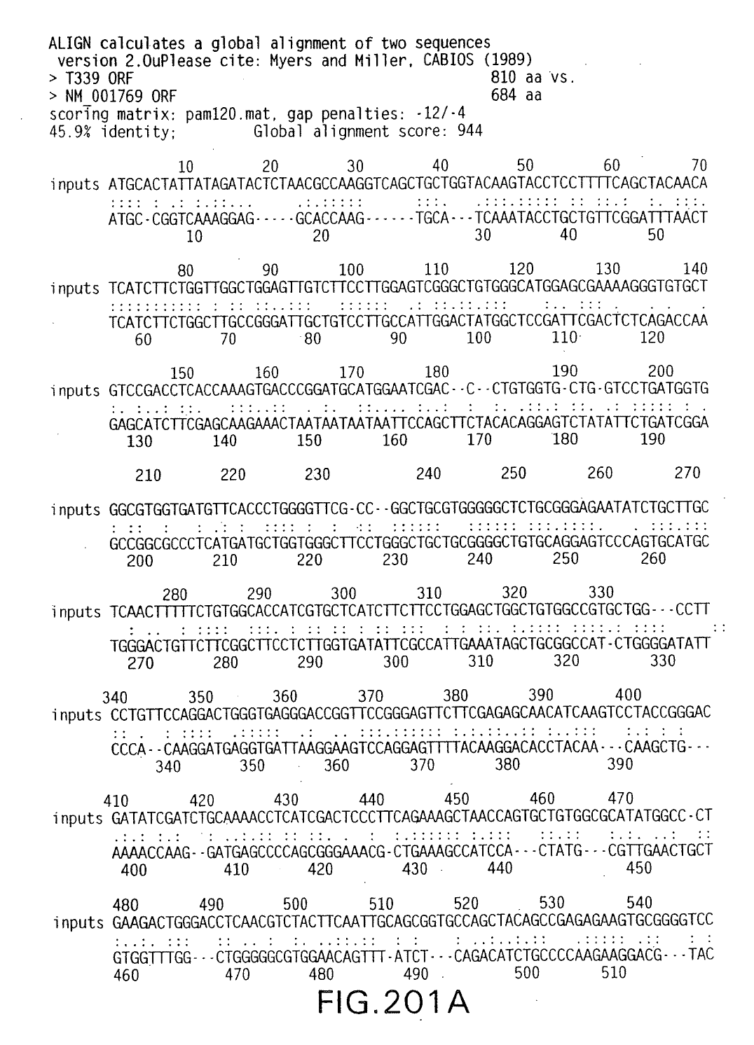

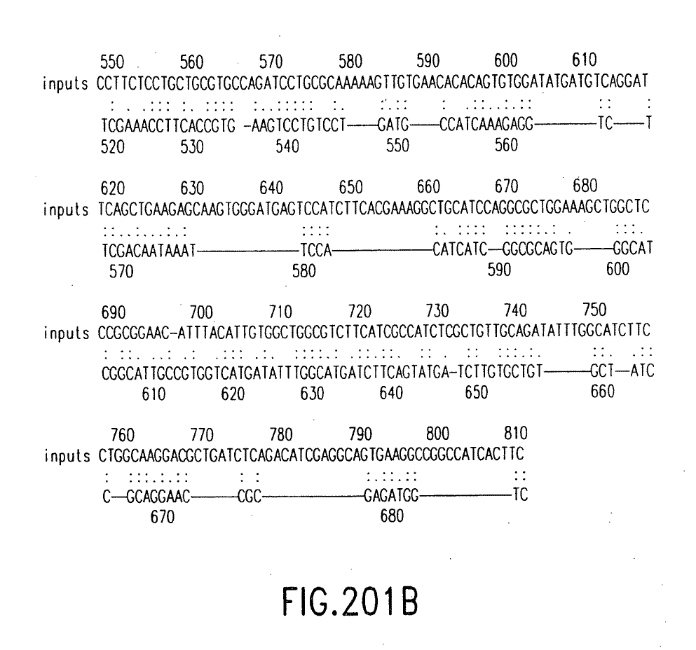

FIG. 201A-201B shows an alignment of the nucleotide sequence of human CD9 antigen coding region (Accession Number NM_{—}001769) and the nucleotide sequence of human TANGO 339 coding region. The alignment shows a 45.9% overall sequence identity between the two nucleotide sequences. The full-length human CD9 antigen nucleic acid sequence (Accession Number NP_{—}001760) and human TANGO 339 cDNA have an overall sequence identity of 30.3%.

Clone EpT339, which encodes human TANGO 339, was deposited with the American Type Culture Collection (10801 University Boulevard, Manassas, Va. 20110-2209) on Jun. 29, 1999 and assigned Accession Number PTA-292. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 339 Nucleic Acids, Polypeptides, and Modulators Thereof

As TANGO 339 was originally found in a human fetal library, TANGO 339 nucleic acids, proteins, and modulators thereof can be used to diagnose disorders and/or modulate the proliferation, development, differentiation, and/or function of cells, tissues and/or organs, e.g., the proliferation of tissues and cells of lymphoid origin and neural origin. TANGO 339 nucleic acids, proteins and modulators thereof can be used to treat immune related disorders, e.g., immunodeficiency disorders (e.g., HIV), viral disorders, cancers, autoimmune disorders, (e.g., arthritis and graft rejection) and inflammatory disorders (e.g., bacterial or viral infection, psoriasis, septicemia, arthritis, allergic reactions). TANGO 339 nucleic acids, proteins, and modulators thereof can be used to diagnose disorders and/or modulate the development of cells, tissues and/or organs in the embryo and/or fetus.

In light of the fact that TANGO 339 has characteristics of transmembrane 4 proteins, TANGO 339 nucleic acids, proteins and modulators thereof can be utilized to modulate (e.g., stabilize, promote, inhibit or disrupt) cellular activation, cellular proliferation, motility, and differentiation. For example, such TANGO 339 compositions and modulators thereof can be used to modulate binding to extracellular matrix (ECM)-associated factors such as integrins and can function to modulate ligand binding to cell surface receptors.

In further light of the fact that TANGO 339 has characteristics of transmembrane 4 proteins, TANGO 339 nucleic acids, proteins and modulators thereof can be used to modulate disorders associated with aberrant signal transduction in response to ECM-associated proteins and cell surface receptors such as other transmembrane 4 proteins. TANGO 339 nucleic acids, proteins and modulators thereof can be utilized to modulate the development and progression of proliferative disorders, e.g., neoplasms or tumors (such as carcinomas, sarcomas, adenomas or myeloid lymphomas) associated with cancer, (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma; leukemias, e.g. acute lymphocytic leukemia and acute myelocytic leukemia (myelolastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's diseases), multiple myeloma and Waldenstrom's macroglobulinemia.

TANGO 339 proteins exhibit similarity to human CD9 antigen, a member of the transmembrane 4 superfamily. In light of this, TANGO 339 nucleic acids, proteins and modulators thereof can be utilized to modulate platelet activation and aggregation. For example, antagonists to TANGO 339 action, such as peptides, antibodies or small molecules that decrease or block TANGO 339 binding to extracellular matrix components (e.g., integrins) or that prevent TANGO 339 signaling, can be used as platelet activation and aggregation blockers. In another example, agonists that mimic TANGO 339 activity, such as peptides, antibodies or small molecules, can be used to induce platelet activation and aggregation. Antibodies may activate or inhibit the cell adhesion, proliferation and activation, and may help in treating inflammation, cancer, cardiovascular disease or stroke by affecting these cellular processes. TANGO 339 nucleic acids, proteins and modulators thereof can be utilized to modulate platelet-related processes and disorders, e.g., Glanzmann's thromboasthemia, which is a bleeding disorder characterized by failure of platelet aggregation in response to cell stimuli.

In further light of the fact that TANGO 339 proteins exhibit similarity to human CD9 antigen, TANGO 339 nucleic acids, proteins and modulators thereof can be utilized to modulate intercellular signaling in the nervous system. The CD9 antigen, which is expressed at the surface of central nervous system (CNS) mature myelin, may modulate intercellular signal transduction and enhance myelin membrane adhesion to extracellular matrices at very late stages of development, thereby playing a role in the maintenance of the entire myelin sheath.

In light, in part, of the fact that TANGO 339 proteins contain peripherin/rom-1-like domains, TANGO 339 nucleic acids, proteins and modulators thereof can be utilized to modulate the development and function of the eye, such as retinal development and function, (e.g., photoreceptor disk morphogenesis). TANGO 339 nucleic acids, proteins and modulators thereof can be utilized to treat eye diseases and/or disorders, e.g., autosomal dominant retinitis pigmentosa, autosomal dominant punctata albescens, butterfly-shaped pigment dystrophy, cataracts, macular degeneration, myopia, stigmatism and retinoblastoma.

As TANGO 339 maps to a region of chromosome 10 which encodes polypeptides expressed in the lung, TANGO 339 nucleic acids, proteins and modulators thereof can be utilized to modulate the development, differentiation and activity of pulmonary structures, e.g., lung. TANGO 339 nucleic acids, proteins and modulators thereof can be utilized to modulate or treat pulmonary disorders, such as atelectasis, pulmonary congestion or edeina, cystic fibrosis, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), lung cancer or tumors (e.g., bronchogenic carcinoma, bronchioloviveolar carcinoma, bronchial carcinoid, hamartoma, and mesenchymal tumors).

As TANGO 339 nucleic acids exhibit homology to a human brain EST (Accession Number Q59384, disclosed in patent No. WP 93/16178), TANGO 339 nucleic acids, proteins and modulators thereof can be utilized to modulate processes involved in the development, differentiation and activity of the brain, including, but not limited to development, differentiation and activation of neuronal cells and glial cells (e.g., oligodendrocytes astrocytes), and amelioration of one or more symptoms associated with abnormal function of such cell types. TANGO 339 nucleic acids, proteins and modulators thereof can be utilized to treat neural diseases and/or disorders, e.g. epilepsy, spinal cord injuries, infarction, infection, malignancy, paraneoplastic syndromes, neuropsychiatric disorders (e.g., schizophrenia, depression, anxiety disorders, and anorexia nervosa), and neurodegenerative diseases including, but not limited to, Alzheimer's disease, Parkinson's disease, Huntington's Chorea, amyotrophic lateral sclerosis and progressive supra-nuclear palsy.

TANGO 339 expression can be utilized as a marker (e.g., an in situ marker) for specific tissues (e.g., the brain) and/or cells (e.g., neurons) in which TANGO 339 is expressed. TANGO 339 nucleic acids can also be utilized for chromosomal mapping, or as chromosomal markers, e.g., in radiation hybrid mapping.

### Human TANGO 358

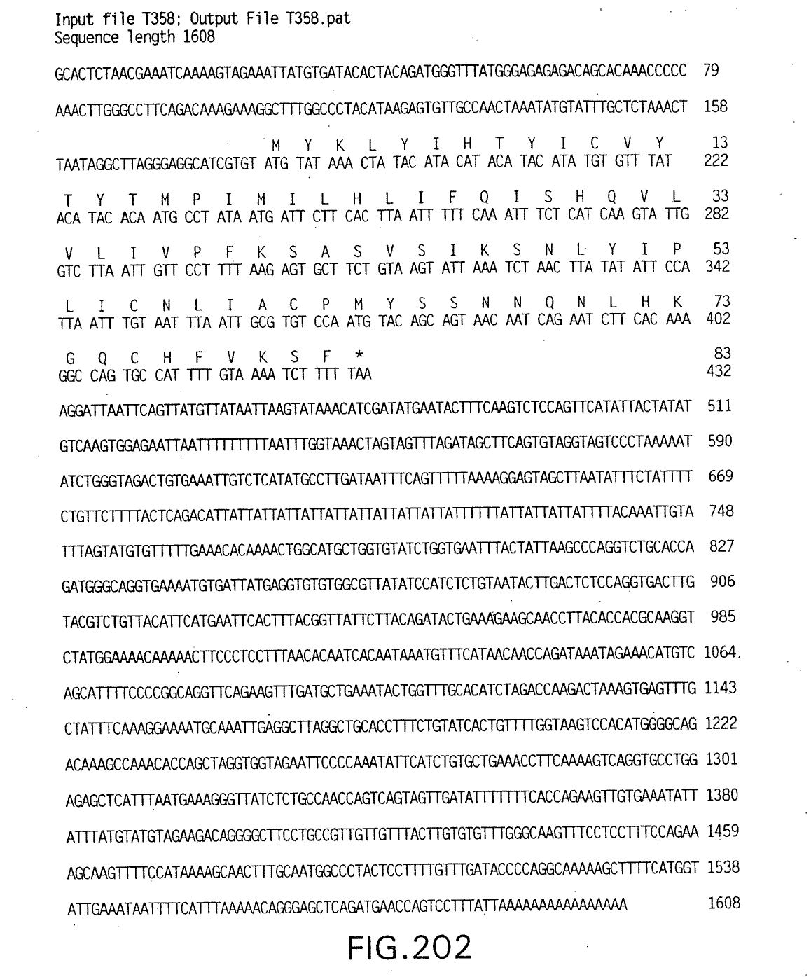

A cDNA encoding human TANGO 358 was identified by analyzing the sequences of clones present in a fetal thymus library for sequences that encode a wholly secreted or transmembrane protein. This analysis led to the identification of a clone, jthTb128c07 encoding full-length human TANGO 358. The human TANGO 358 cDNA of this clone is 1608 nucleotides long (FIG. 202; SEQ ID NO: 127). The open reading frame of this cDNA (nucleotides 184 to 429 of SEQ ID NO: 127) encodes a 82 amino acid transmembrane protein (SEQ ID NO:128).

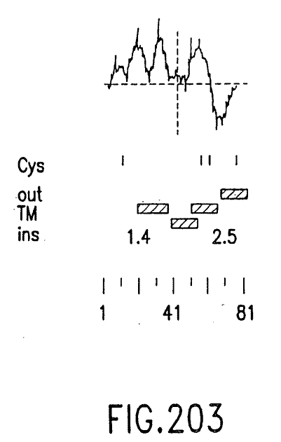

FIG. 203 depicts a hydropathy plot of human TANGO 358.

The signal peptide prediction program SIGNALP (Nielsen et al., 1997, Protein Engineering 10:1-6) predicted that human TANGO 358 includes a 42 amino acid signal peptide (amino acid 1 to amino acid 42) preceding the mature human TANGO 358 protein (corresponding to amino acid 43 to amino acid 82). The molecular weight of human TANGO 358 protein without post-translational modifications is 9.5 kDa prior to the cleavage of the signal peptide and 4.5 kDa after cleavage of the signal peptide. The presence of a methionine residue at positions 17, 20 and 63 indicates that there can be alternative forms of human TANGO 358 of 66 amino acids, 63 amino acids, and 20 amino acids.

Human TANGO 358 is a transmembrane protein which can include one or more of the following domains: (1) an extracellular domain; (2) a transmembrane domain; and (3) a cytoplasmic domain. The human TANGO 358 protein contains an extracellular domain at amino acid residues 43 to 49, a transmembrane domain at amino acid residues 50 to 66, and a cytoplasmic domain at amino acid residues 67 to 82 of SEQ ID NO:128.

Alternatively, in another embodiment, a human TANGO 358 protein contains a cytoplasmic domain at amino acid residues 43 to 49, a transmembrane domain at amino acid to residues 50 to 66, and an extracellular domain at amino acid residues 67 to 82. Further, human TANGO 358 has a protein kinase C phosphorylation site with the sequence SIK (at amino acid residues 45 to 47).

In one embodiment of a nucleotide sequence of human TANGO 358, the nucleotide at position 20 is adenine (A). In this embodiment, the amino acid at position 7 is histidine (H). In an alternative embodiment, a species variant of human TANGO 358 has a nucleotide at position 20 which is guanine (G). In this embodiment, the amino acid at position 7 is arginine (R), i.e., a conservative substitution.

In one embodiment of a nucleotide sequence of human TANGO 358, the nucleotide at position 35 is thymine (T). In this embodiment, the amino acid at position 12 is valine (V). In an alternative embodiment, a species variant of human TANGO 358 has a nucleotide at position 35 which is cytosine (C). In this embodiment, the amino acid at position 12 is alanine (A), i.e., a conservative substitution.

In one embodiment of a nucleotide sequence of human TANGO 358, the nucleotide at position 85 is thymine (T). In this embodiment, the amino acid at position 29 is serine (S). In an alternative embodiment, a species variant of human TANGO 358 has a nucleotide at position 85 which is adenine (A). In this embodiment, the amino acid at position 29 is threonine (T), i.e., a conservative substitution.

In one embodiment of a nucleotide sequence of human TANGO 358, the nucleotide at position 91 is cytosine (C). In this embodiment, the amino acid at position 31 is glutamine (Q). In an alternative embodiment, a species variant of human TANGO 358 has a nucleotide at position 91 which is guanine (G). In this embodiment, the amino acid at position 31 is glutamate (E), i.e., a conservative substitution.

Clone EpT358, which encodes human TANGO 358, was deposited with the American Type Culture Collection (10801 University Boulevard, Manassas, Va. 20110-2209) on Jun. 29, 1999, and assigned Accession Number PTA-292. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 358 Nucleic Acids, Polypeptides, and Modulators Thereof

As TANGO 358 was originally found in a fetal thymus library, TANGO 358 nucleic acids, proteins, and modulators thereof can be used to diagnose thymus associated disorders. TANGO 358 nucleic acids, proteins, and modulators thereof can also be used modulate the proliferation, development, differentiation, maturation and/or function of thymocytes, e.g., modulate development and maturation of T-lymphocytes. TANGO 358 nucleic acids, proteins and modulators thereof can be utilized to modulate immune-related processes such as the ability to modulate host immune response by, e.g., modulating the formation of and/or binding to immune complexes, and modulating the positive and negative selection of thymocytes. Such TANGO 358 compositions and modulators thereof can be utilized, e.g., to ameliorate incidence of any symptoms associated with disorders that involve such immune-related processes, including, but not limited to infection and autoimmune disorders (e.g., insulin-dependent mellitus, multiple sclerosis, systemic lupus, erythematosus, sjogren's syndrome, autoimmune thyroiditis, idiotpathic Addison's disease, vitiligo, Grave's disease, idiopathic thrombocytopenia purpura, rheumatoid arthritis, and scleroderma). TANGO 358 nucleic acids, proteins and modulators thereof can also be utilized to treat viral infections, inflammatory immune disorders and immune-related cancers including but not limited to, leukemia (e.g., acute leukemia, chronic leukemia, Hodgkin's disease non-Hodgkin's lymphoma, and multiple myeloma).

Disorders associated with TANGO 358 activity, including those which TANGO 358 proteins, nucleic acids and modulators thereof may be an antagonist can be used to treat include immune disorders, e.g., autoimmune disorders (e.g., arthritis, graft rejection (e.g., allograft rejection), T cell disorders (e.g., AIDS)) and inflammatory disorders (e.g., bacterial infection, psoriasis, septicemia, cerebral malaria, inflammatory bowel disease, arthritis (e.g., rheumatoid arthritis, osteoarthritis), and allergic inflammatory disorders (e.g., asthma, psoriasis)). Disorders associated with modulated TANGO 358 activity can also include apoptotic disorders (e.g., rheumatoid arthritis, systemic lupus erythematosus, insulin-dependent diabetes mellitus), cytotoxic disorders, septic shock, cachexia, and proliferative disorders (e.g., B cell cancers stimulated by TNF).

In light of the fact that TANGO 358 was isolated from a thymus library, TANGO 358 proteins, nucleic acids and modulators thereof can be used to treat disorders that include TNF-related disorders (e.g., acute myocarditis, myocardial infarction, congestive heart failure, T cell disorders (e.g., dermatitis, fibrosis)), differentiative and apoptotic disorders, and disorders related to angiogenesis (e.g., tumor formation and/or metastasis, cancer). Modulators of TANGO 358 expression and/or activity can be used to treat such disorders.

As TANGO 358 is a transmembrane protein, TANGO 358 nucleic acids, proteins and modulators thereof can be utilized to diagnose disorders and/or modulate intercellular signaling pathways, for example by disrupting ligand-receptor interactions or cellular interactions with the extra-cellular matrix.

TANGO 358 expression can be utilized as a marker (e.g., an in situ marker) for specific tissues (e.g., the thymus) and/or cells (e.g., T-lymphocytes) in which TANGO 358 is expressed. TANGO 358 nucleic acids can also be utilized for chromosomal mapping, or as chromosomal markers, e.g., in radiation hybrid mapping.

### Human TANGO 365

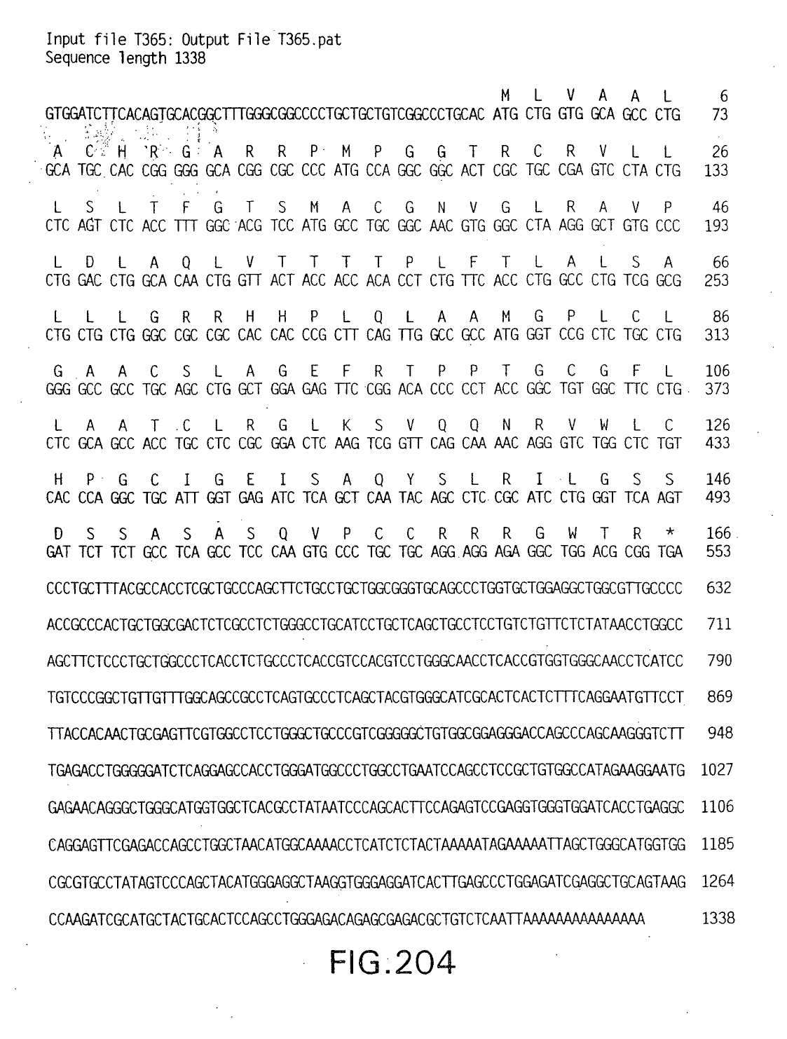

A cDNA encoding TANGO 365 was identified by analyzing the sequences of clones present in a human prostate fibroblast library for sequences that encode wholly secreted or transmembrane proteins. This analysis led to the identification of a clone, jthqc001g06, encoding full-length human TANGO 365. The TANGO 365 cDNA of this clone is 1338 nucleotides long (FIG. 204; SEQ ID NO: 129). The open reading frame of this cDNA (nucleotides 56 to 550 of SEQ ID NO: 129) encodes a 165 amino acid transmembrane protein (SEQ ID NO: 130).

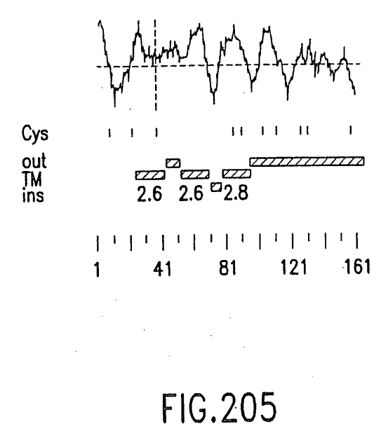

FIG. 205 depicts a hydropathy plot of human TANGO 365. The dashed vertical line separates the signal sequence (amino acids 1 to 36 of SEQ ID NO:130) on the left from the mature protein (amino acids 37 to 165 of SEQ ID NO:130) on the right.

The signal peptide prediction program SIGNALP (Nielsen et al., 1997, Protein Engineering 10:1-6) predicted that human TANGO 365 includes a 36 amino acid signal peptide (amino acid 1 to amino acid 36) preceding the mature protein (corresponding to amino acid 37 to amino acid 165). The molecular weight of TANGO 365 protein without post-translational modifications is 17.4 kDa prior to the cleavage of the signal peptide, 13.6 kDa after cleavage of the signal peptide. The presence of a methionine residue at positions 16, 35 and 81 indicates that there can be alternative forms of human TANGO 365 of 150 amino acids, 131 amino acids, and 65 amino acids, respectively.

Human TANGO 365 is a transmembrane protein which can include one or more of the following domains: (1) an extracellular domain; (2) a transmembrane domain; and (3) a cytoplasmic domain. The human TANGO 365 protein contains two extracellular domains; one at amino acid residues 37 to 51; and a second at amino acid residues 95 to 165, two hydrophobic transmembrane domains; one at amino acids 52 to 70; and a second at amino acids 78 to 94, and a cytoplasmic domain at amino acid residues 71 to 77 of SEQ ID NO: 130.

Alternatively, in another embodiment, a human TANGO 365 protein contains two cytoplasmic domains; one at amino acid residues 37 to 51; and a second at amino acid residues 95 to 165, two hydrophobic transmembrane domains; one at amino acids 52 to 70; and a second at amino acids 78 to 94, and an extracellular domain at amino acid residues 71 to 77 of SEQ ID NO:130.

In one embodiment of a nucleotide sequence of human TANGO 365, the nucleotide at position 14 is cytosine (C). In this embodiment, the amino acid at position 5 is alanine (A). In an alternative embodiment, a species variant of human TANGO 365 has a nucleotide at position 14 which is thymidine (T). In this embodiment, the amino acid at position 5 is valine (V), i.e., a conservative substitution.

In one embodiment of a nucleotide sequence of human TANGO 365, the nucleotide at position 41 is guanine (G). In this embodiment, the amino acid at position 14 is arginine (R). In an alternative embodiment, a species variant of human TANGO 365 has a nucleotide at position 41 which is adenine (A). In this embodiment, the amino acid at position 14 is histidine (H), i.e., a conservative substitution.

In one embodiment of a nucleotide sequence of human TANGO 365, the nucleotide at position 59 is cytosine (C). In this embodiment, the amino acid at position 20 is threonine (T). In an alternative embodiment, a species variant of human TANGO 365 has a nucleotide at position 59 which is guanine (G). In this embodiment, the amino acid at position 20 is serine (S), i.e., a conservative substitution.

In one embodiment of a nucleotide sequence of human TANGO 365, the nucleotide at position 115 is adenine (A). In this embodiment, the amino acid at position 39 is asparagine (N). In an alternative embodiment, a species variant of human TANGO 365 has a nucleotide at position 115 which is guanine (G). In this embodiment, the amino acid at position 39 is aspartate (D), i.e., a conservative substitution.

One protein kinase C phosphorylation site is present in human TANGO 365. The site has the sequence SLR and is found (at amino acids 139 to 141). The TANGO 365 protein has four N-myristoylation sites. The first has the sequence GGTRCR and is found (at amino acids 18 to 23), the second has the sequence GTSMAC and is found (at amino acids 32 to 37), the third has the sequence GAACSL and is found (at amino acids 87 to 92), and the fourth has the sequence GSSDSS and is found (at amino acids 144 to 149). Human TANGO 365 also has an amidation site which has the sequence of LGRR (at amino acids 69 to 72).

Clone EpT365, which encodes human TANGO 365, was deposited with the American Type Culture Collection (10801 University Boulevard, Manassas, Va. 20110-2209) on Jun. 29, 1999 and assigned Accession Number PTA-291. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 365 Nucleic Acids, Polypeptides, and Modulators Thereof

TANGO 365 was identified as being expressed in a prostate fibroblast library. In light of this, TANGO 365 nucleic acids, proteins and modulators thereof can be utilized to diagnose disorders and/or modulate processes involved in prostate development, differentiation and activity, including, but not limited to development, and differentiation and activation of prostate tissues and cells as well as any function associated with such cells, and amelioration of one or more symptoms associated with abnormal function of such cell types. Such disorders can include, but are not limited to, malignant or benign prostate cell growth. Such disorders can include, but are not limited to, malignant or benign prostate cell growth. The TANGO 365 proteins can be used to treat subjects with or without prostate cancer e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, atypical prostatic stromal lesions.

TANGO 365 nucleic acids, proteins, and modulators thereof can also be used to treat disorders of the cells and tissues in which it is expressed. As TANGO 365 is a transmembrane protein, proteins, nucleic acids and modulators thereof can be used to diagnose disorders and/or modulate intercellular signaling processes by disrupting or enhancing ligand-receptor or cell interaction with the extracellular matrix. Further, TANGO 365 could be used in detection and diagnostic assays to assay for normal or inappropriate expression of TANGO 365 proteins in aberrantly growing cells.

TANGO 365 expression can be utilized as a marker (e.g., an in situ marker) for specific tissues (e.g., the prostate) and/or cells (e.g., fibroblasts) in which TANGO 365 is expressed. TANGO 365 nucleic acids can also be utilized for chromosomal mapping, or as chromosomal markers, e.g., in radiation hybrid mapping.

### Human TANGO 368

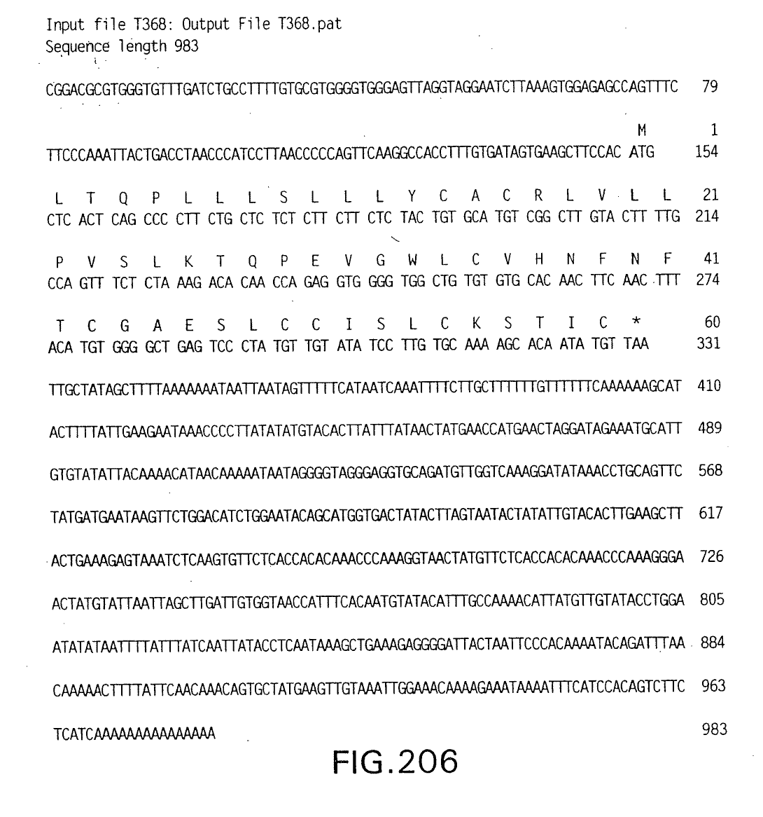

A cDNA encoding human TANGO 368 was identified by analyzing the sequences of clones present in a natural killer cell library for sequences that encode wholly secreted or transmembrane proteins. This analysis led to the identification of a clone, jthta080f06, encoding full-length human TANGO 368. The human TANGO 368 cDNA of this clone is 983 nucleotides long (FIG. 206; SEQ ID NO: 131). The open reading frame of this cDNA (nucleotides 152 to 328 of SEQ ID NO: 131) encodes a 59 amino acid secreted protein (SEQ ID NO:132).

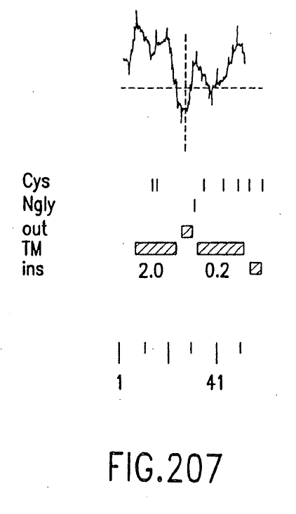

FIG. 207 depicts a hydropathy plot of human TANGO 368.

The signal peptide prediction program SIGNALP (Nielsen et al., 1997, Protein Engineering 10:1-6) predicted that human TANGO 368 includes a 26 amino acid signal peptide (amino acid 1 to amino acid 27 of SEQ ID NO:132) preceding the mature human TANGO 368 protein (corresponding to amino acid 28 to amino acid 59 of SEQ ID NO:132). The molecular weight of TANGO 368 protein without post-translational modifications is 6.5 kDa prior to the cleavage of the signal peptide and 3.5 kDa after cleavage of the signal peptide.

In one embodiment of a nucleotide sequence of human TANGO 368, the nucleotide at position 8 is cytosine (C). In this embodiment, the amino acid at position 3 is threonine (T). In an alternative embodiment, a species variant of human TANGO 368 has a nucleotide at position 8 which is guanine (G). In this embodiment, the amino acid at position 3 is serine (S), i.e., a conservative substitution.

In one embodiment of a nucleotide sequence of human TANGO 368, the nucleotide at position 10 is cytosine (C). In this embodiment, the amino acid at position 4 is glutamine (Q). In an alternative embodiment, a species variant of human TANGO 368 has a nucleotide at position 10 which is guanine (G). In this embodiment, the amino acid at position 4 is glutamate (E), i.e., a conservative substitution.

In one embodiment of a nucleotide sequence of human TANGO 368, the nucleotide at position 16 is cytosine (C). In this embodiment, the amino acid at position 6 is leucine (L). In an alternative embodiment, a species variant of human TANGO 368 has a nucleotide at position 16 which is guanine (G). In this embodiment, the amino acid at position 6 is valine (V), i.e., a conservative substitution.

In one embodiment of a nucleotide sequence of human TANGO 368, the nucleotide at position 110 is adenine (A). In this embodiment, the amino acid at position 37 is histidine (H). In an alternative embodiment, a species variant of human TANGO 368 has a nucleotide at position 110 which is guanine (G). In this embodiment, the amino acid at position 37 is arginine (R), i.e., a conservative substitution.

Human TANGO 368 has an N-glycosylation site with the sequence NFTC (at amino acid residues 40 to 43), a protein kinase C phosphorylation site with the sequence SLK (at amino acid residues 24 to 26), and a casein kinase II phosphorylation site with the sequence TQPE (at amino acid residues 27 to 30).

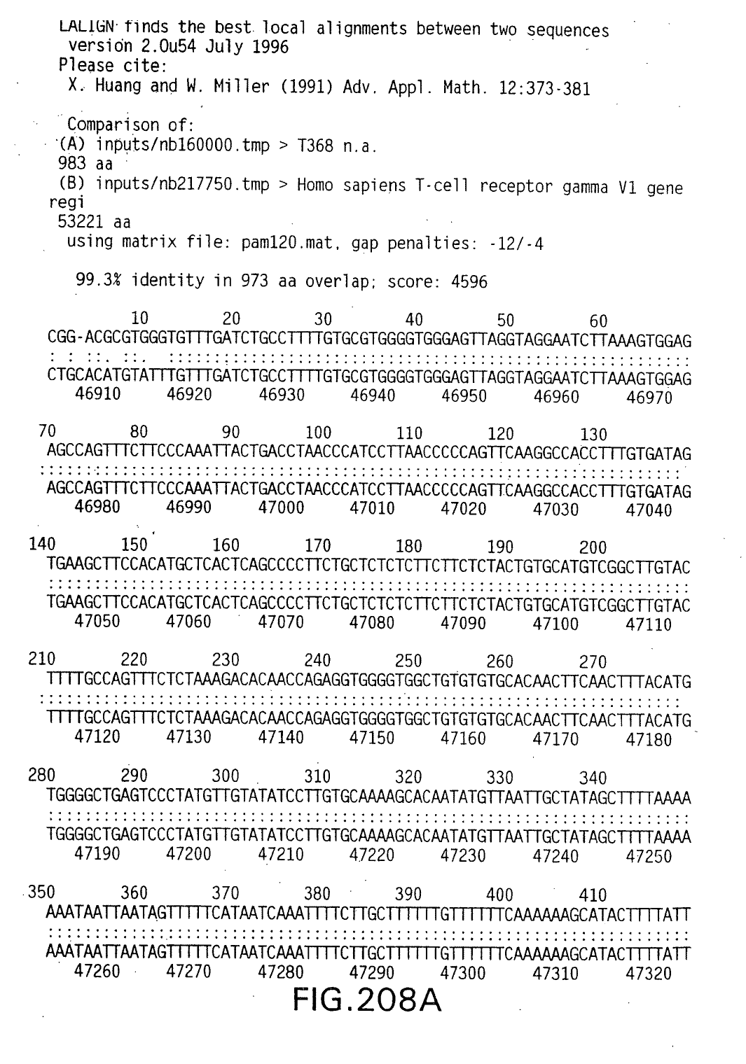

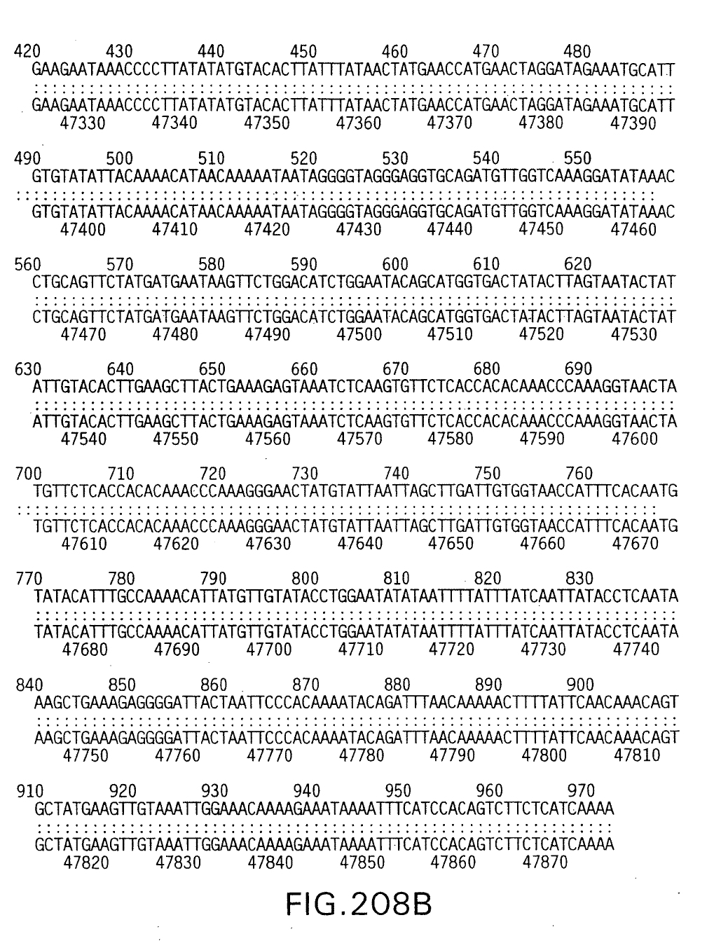

FIG. 208A-208B depicts a local alignment of the nucleotide sequence of full length human TANGO 368 and a fragment of the human T-cell receptor gamma V1 gene region (Accession Number AF057177), which maps to a region of human chromosome 7. The full-length nucleic acid sequence of human TANGO 368 has 99.3% identity to a 973 bp fragment of the human T-cell receptor gamma V1 gene region (Accession Number AF057177).

Northern blots were performed to analyze the expression of human TANGO 368 mRNA in human tissues. A weak signal was observed in the spleen and lymph node, however, no expression was detected in the thymus, peripheral blood leukocytes or fetal liver.

Clone EpT368, which encodes human TANGO 368, was deposited with the American Type Culture Collection (10801 University Boulevard, Manassas, Va. 20110-152209) on Jun. 29, 1999 and assigned Accession Number PTA-291. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 368 Nucleic Acids, Polypeptides, and Modulators Thereof

As TANGO 368 was originally found in a natural killer cell library, TANGO 368 nucleic acids, proteins, and modulators thereof can be used to diagnose disorders and/or modulate the proliferation, development, differentiation, and/or function of immune cells, such as lymphocytes, e.g., natural killer cells, T-cells and B-cells. TANGO 368 nucleic acids, proteins and modulators thereof can be utilized to modulate immune-related processes e.g., the host immune response by, for example, modulating the formation of and/or binding to immune complexes, detection and defense against surface antigens and bacteria, and immune surveillance for rapid removal or pathogens. Such TANGO 368 nucleic acids, proteins and modulators thereof can be utilized, e.g., to ameliorate incidence of any symptoms associated with disorders that involve such immune-related processes, including, but not limited to viral or bacterial infection, autoimmune disorders (e.g., Grave's disease, Hashimoto's disease, and arthritis), immunodeficiency disorders (e.g., HIV, and inflammatory disorders (e.g., asthma, arthritis, psoriasis, septicemia, inflammatory bowel disease and allergies).

As TANGO 368 exhibits expression in the spleen, TANGO 368 nucleic acids, proteins, and modulators thereof can be used to diagnose disorders and/or modulate the proliferation, differentiation, and/or function of cells that form the spleen, e.g., cells of the splenic connective tissue, e.g., splenic smooth muscle cells and/or endothelial cells of the splenic blood vessels. TANGO 368 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 368 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 368 exhibits expression in the lymph nodes, TANGO 368 nucleic acids, proteins, and modulators thereof can be used to diagnose disorders and/or modulate the proliferation, differentiation, and/or function of cells that form the lymph node, e.g., cells of the lymph node connective tissue, e.g., lymph node smooth muscle cells and/or endothelial cells of the lymph node blood vessels. TANGO 368 nucleic acids, proteins, and modulators thereof can also be used to diagnose disorders and/or modulate the proliferation, differentiation, and/or function of cells that are processed, e.g., phagocytized within the lymph node, e.g., erythrocytes and/or B and T lymphocytes and macrophages. Thus, TANGO 368 nucleic acids, proteins, and modulators thereof can be used to treat lymph node associated diseases and disorders. Examples of lymph node diseases and disorders include e.g., lymphadenopathy, lymphoma, and/or phagocytotic disorders, e.g., those inhibiting macrophage engulfment of bacteria and viruses in the bloodstream.

In light of the fact that TANGO 368 is homologous to the T-cell receptor gamma (TCRγ) locus, TANGO 368 nucleic acids, proteins and modulators thereof can be utilized to modulate the recognition of antigens in association with the major histocompatibility complex. TANGO 368 nucleic acids, proteins and modulators thereof can be utilized to modulate diseases and/or disorders associated with aberrant TCR-MHC interactions. Further, TANGO 368 nucleic acids, proteins and modulators thereof can be utilized to modulate cell-cell receptor interactions.

As TANGO 368 exhibits homology to human T-cell receptor gamma V1 gene region (Accession Numbers AF057177), which maps to a region of chromosome 7, TANGO 368 nucleic acids, proteins and modulators thereof can be utilized to diagnose disorders and/or modulate diseases associated with that region of chromosome 7, e.g., Stiff-Mann syndrome.

As TANGO 368 is a secreted protein and thus likely a signaling molecule, TANGO 368 nucleic acids, proteins or modulators thereof, can be used to modulate TANGO 368 biological activities, which include, e.g., (1) the ability to modulate, e.g. stabilize, promote, inhibit or disrupt, protein-protein interactions (e.g., homophilic and/or heterophilic), and protein-ligand interactions, e.g., in receptor-ligand recognition; (2) ability to modulate cell-cell interactions; (3) the ability to modulate the proliferation, differentiation and/or activity of neural cells; and (4) the ability to modulate intracellular signaling cascades (e.g., signal transduction cascades).

TANGO 368 expression can be utilized as a marker (e.g., an in situ marker) for specific tissues (e.g., the thymus) and/or cells (e.g., natural killer cells) in which TANGO 368 is expressed. TANGO 368 nucleic acids can also be utilized for chromosomal mapping, or as chromosomal markers, e.g., in radiation hybrid mapping.

### Human TANGO 369

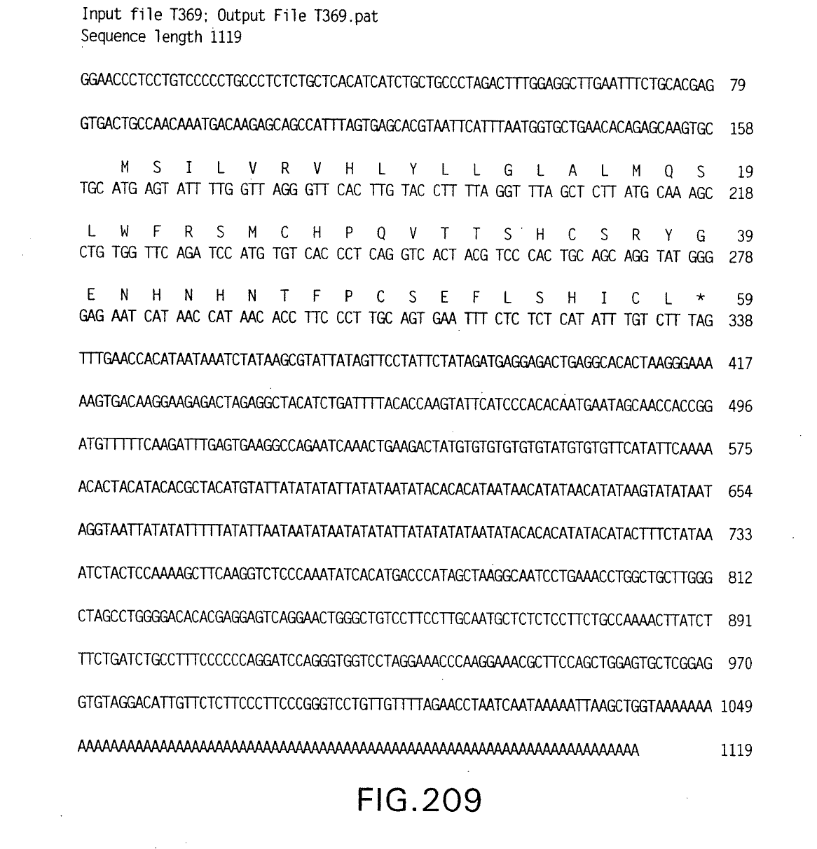

A cDNA encoding human TANGO 369 was identified by analyzing the sequences of clones present in a natural killer cell library for sequences that encode wholly secreted or transmembrane proteins. This analysis led to the identification of a clone, jthta088h08, encoding full-length human TANGO 369. The human TANGO 369 cDNA of this clone is 1119 nucleotides long (FIG. 209; SEQ ID NO: 133). The open reading frame of this cDNA (nucleotides 162 to 335 of SEQ ID NO: 133) encodes a 58 amino acid secreted protein (SEQ ID NO:134).

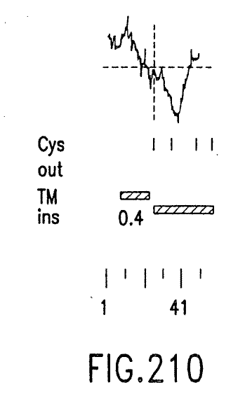

FIG. 210 depicts a hydropathy plot of human TANGO 369.

The signal peptide prediction program SIGNALP (Nielsen et al., 1997, Protein Engineering 10: 1-6) predicted that human TANGO 369 includes a 26 amino acid signal peptide (amino acid 1 to amino acid 26) preceding the mature human TANGO 369 protein (corresponding to amino acid 27 to amino acid 58). The molecular weight of TANGO 369 protein without post-translational modifications is 6.8 kDa prior to the cleavage of the signal peptide and 3.7 kDa after cleavage of the signal peptide. The presence of a methionine residue at positions 17 and 250 indicates that there can be alternative forms of human TANGO 369 of 42 amino acids of SEQ ID NO:134.

In one embodiment of a nucleotide sequence of human TANGO 369, the nucleotide at position 58 is cytosine (C). In this embodiment, the amino acid at position 20 is leucine (L). In an alternative embodiment, a species variant of human TANGO 369 has a nucleotide at position 58 which is guanine (G). In this embodiment, the amino acid at position 20 is valine (V), i.e., a conservative substitution.

In one embodiment of a nucleotide sequence of human TANGO 369, the nucleotide at position 68 is guanine (G). In this embodiment, the amino acid at position 23 is arginine (R). In an alternative embodiment, a species variant of human TANGO 369 has a nucleotide at position 68 which is adenine (A). In this embodiment, the amino acid at position 23 is lysine (K), i.e., a conservative substitution.

In one embodiment of a nucleotide sequence of human TANGO 369, the nucleotide at position 70 is thymine (T). In this embodiment, the amino acid at position 24 is leucine (L). In an alternative embodiment, a species variant of human TANGO 369 has a nucleotide at position 70 which is adenine (A). In this embodiment, the amino acid at position 24 is threonine (T), i.e., a conservative substitution.

In one embodiment of a nucleotide sequence of human TANGO 369, the nucleotide at position 120 is guanine (G). In this embodiment, the amino acid at position 40 is glutamate (E). In an alternative embodiment, a species variant of human TANGO 369 has a nucleotide at position 120 which is cytosine (C). In this embodiment, the amino acid at position 40 is aspartate (D), i.e., a conservative substitution.

Northern blots were performed to analyze the expression of human TANGO 369 mRNA in human tissues. A very weak signal was observed in the spleen and lymph node, however, no expression was detected in the thymus, peripheral blood leukocytes or fetal liver.

Clone EpT369, which encodes human TANGO 369, was deposited with the American Type Culture Collection (10801 University Boulevard, Manassas, Va. 20110-2209) on Jun. 29, 1999 and assigned Accession Number PTA-295. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 369 Nucleic Acids, Polypeptides, and Modulators Thereof

As TANGO 369 was originally found in a natural killer cell library, TANGO 369 nucleic acids, proteins, and modulators thereof can be used to diagnose disorders and/or modulate the proliferation, development, differentiation, and/or function of lymphocytes, e.g., natural killer cells. TANGO 369 nucleic acids, proteins and modulators thereof can be utilized to modulate immune-related processes, e.g., the host immune response by, for example, modulating the formation of and/or binding to immune complexes, detection and defense against surface antigens and bacteria, and immune surveillance for rapid removal or pathogens. Such TANGO 369 compositions and modulators thereof can be utilized, e.g., to ameliorate incidence of any symptoms associated with disorders that involve such immune-related processes, including, but not limited to viral or bacterial infection, autoimmune disorders (e.g., Grave's disease, Hashimoto's disease, arthritis, graft rejection), and inflammatory disorders (e.g., bacterial or viral infection, psoriasis, allergies and inflammatory bowel diseases).

As TANGO 369 exhibits expression in the spleen, TANGO 369 nucleic acids, proteins, and modulators thereof can be used to diagnose disorders and/or modulate the proliferation, differentiation, and/or function of cells that form the spleen, e.g., cells of the splenic connective tissue, e.g., splenic smooth muscle cells and/or endothelial cells of the splenic blood vessels. TANGO 369 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, TANGO 369 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

As TANGO 369 exhibits expression in the lymph nodes, TANGO 369 nucleic acids, proteins, and modulators thereof can be used to diagnose disorders and/or modulate the proliferation, differentiation, and/or function of cells that form the lymph node, e.g., cells of the lymph node connective tissue, e.g., lymph node smooth muscle cells and/or endothelial cells of the lymph node blood vessels. TANGO 369 nucleic acids, proteins, and modulators thereof can also be used to modulate the proliferation, differentiation, and/or function of cells that are processed, e.g., phagocytized within the lymph node, e.g., erythrocytes and/or B and T lymphocytes and macrophages. Thus, TANGO 369 nucleic acids, proteins, and modulators thereof can be used to treat lymph node associated diseases and disorders. Examples of lymph node diseases and disorders include e.g., lymphadenopathy, lymphoma, and/or phagocytotic disorders, e.g., those inhibiting macrophage engulfment of bacteria and viruses in the bloodstream.

TANGO 369 is associated with immune cells. As such, immune disorders associated TANGO 369 nucleic acids, proteins and modulators thereof can be used to diagnose disorders and/or modulate or treat immune disorders that include, but are not limited to, immune proliferative disorders (e.g., carcinoma, lymphoma, e.g., follicular lymphoma), and disorders associated with fighting pathogenic infections, e.g., bacterial (e.g., chlamydia) infection, parasitic infection, and viral infection (e.g., HSV infection), and pathogenic disorders associated with immune disorders (e.g., immunodeficiency disorders, such as HIV).

Other immune disorders associated with TANGO 369 activity, for which TANGO 369 nucleic acids, proteins and modulators thereof can be used to modulate, identify, diagnose or treat, include, e.g., autoimmune disorders, such as arthritis, graft rejection (e.g., allograft rejection), T cell disorders (e.g., AIDS)) and inflammatory disorders, such as bacterial infection, psoriasis, septicemia, cerebral malaria, inflammatory bowel disease, arthritis (e.g., rheumatoid arthritis, osteoarthritis), and allergic inflammatory disorders (e.g., asthma, psoriasis), apoptotic disorders (e.g. rheumatoid arthritis, systemic lupus erythematosus, insulin-dependent diabetes mellitus), cytotoxic disorders, septic shock, cachexia, and proliferative disorders (e.g., B cell cancers stimulated by TNF).

Other TANGO 369 associated immune disorders include TNF related disorders (e.g., acute myocarditis, myocardial infarction, congestive heart failure, T cell disorders (e.g., dermatitis, fibrosis)), differentiative and apoptotic disorders, and disorders related to angiogenesis (e.g., tumor formation and/or metastasis, cancer). TANGO 369 nucleic acids, proteins and modulators thereof can be used to treat such disorders.

As TANGO 369 is a secreted protein, TANGO 369 nucleic acids, proteins and modulators thereof can be utilized to modulate intercellular signaling pathways, for example by disrupting ligand-receptor interactions or cellular interactions with the extra-cellular matrix.

As TANGO 369 is a secreted protein and thus likely a signaling molecule, TANGO 369 nucleic acids, proteins or modulators thereof can be used TANGO 369 biological activities, which can also include, e.g., (1) the ability to modulate, e.g., stabilize, promote, inhibit or disrupt, protein-protein interactions (e.g., homophilic and/or heterophilic), and protein-ligand interactions, e.g., in receptor-ligand recognition; (2) ability to modulate cell-cell interactions; (3) the ability to modulate the proliferation, differentiation and/or activity of neural cells; and (4) the ability to modulate intracellular signaling cascades (e.g., signal transduction cascades).

TANGO 369 expression can be utilized as a marker (e.g., an in situ marker) for specific tissues (e.g., the thymus) and/or cells (e.g., natural killer cells) in which TANGO 369 is expressed. TANGO 369 nucleic acids can also be utilized for chromosomal mapping, or as chromosomal markers, e.g., in radiation hybrid mapping.

### Human TANGO 383

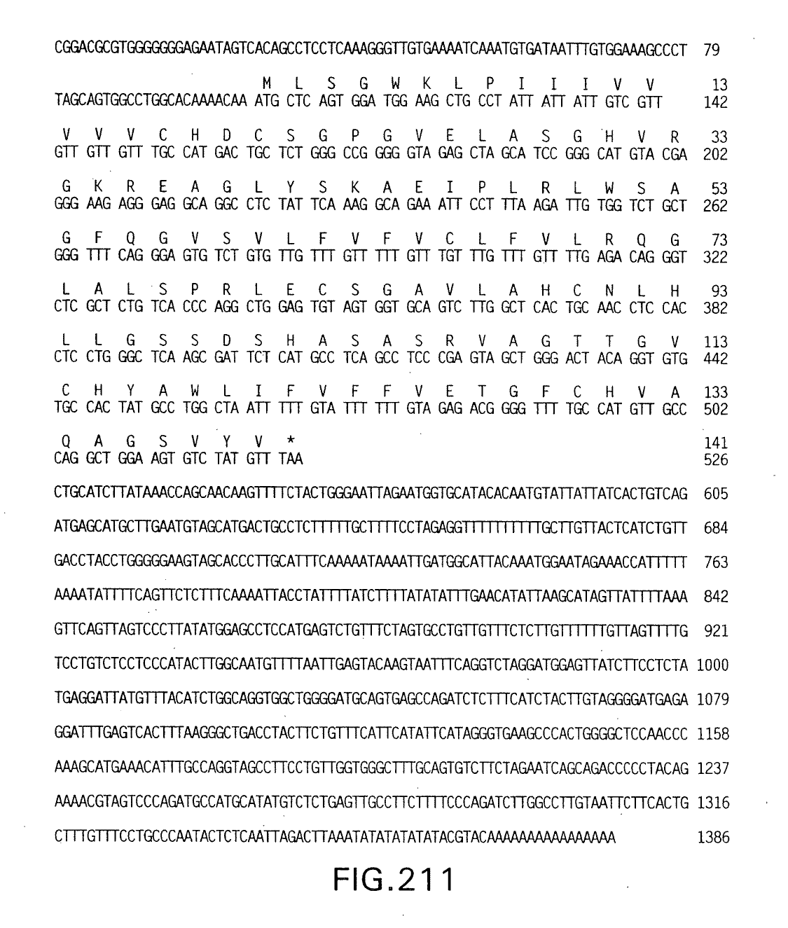

A cDNA encoding human TANGO 383 was identified by analyzing the sequences of clones present in a human prostate epithelium cDNA library. This analysis led to the identification of a clone, jthqb083b10, encoding full-length TANGO 383. The human cDNA of this clone is 1386 nucleotides long (FIG. 211; SEQ ID NO:135). The open reading frame of this cDNA (nucleotides 104 to 523 of SEQ ID NO: 135) encodes a 140 amino acid TANGO 383 transmembrane protein (SEQ ID NO:136).

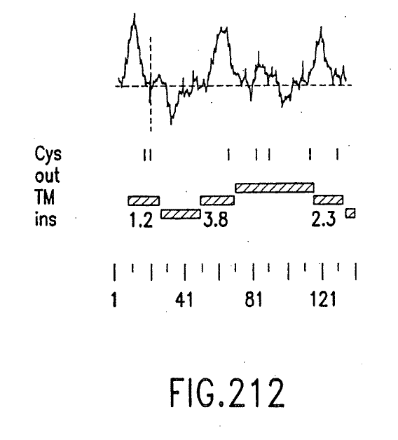

FIG. 212 depicts a hydropathy plot of human TANGO 383. The dashed vertical line separates the signal sequence (amino acids 1 to of SEQ ID NO: 136) on the left from the mature protein (amino acids 21 to 140 of SEQ ID NO: 136) on the right.

The signal peptide prediction program SIGNALP (Nielsen, et al. (1997) Protein Engineering 10:1-6) predicted that TANGO 383 includes a 20 amino acid signal peptide (amino acid 1 to amino acid 20 of SEQ ID NO: 136) preceding the mature protein (corresponding to amino acid 21 to amino acid 140 of SEQ ID NO: 136). The molecular weight of TANGO 383 without post-translational modifications is 14.9 kDa prior to the cleavage of the signal peptide, 12.7 kDa after cleavage of the signal peptide.

TANGO 383 is a transmembrane protein which contains one or more of the following domains: (1) an extracellular domain; (2) a transmembrane domain; and (3) a cytoplasmic domain. The TANGO 383 protein contains an extracellular domain at amino acids 71 to 115, a first transmembrane domain at amino acid residues 50 to 70, a second transmembrane domain at amino acid residues 116 to 133, a first cytoplasmic domain at amino acid residues 21 to 49 and a second cytoplasmic domain at amino acid residues 134 to 140 of SEQ ID NO: 136.

Alternatively, in another embodiment, a TANGO 383 protein contains a cytoplasmic domain at amino acids 71 to 115, a first transmembrane domain at amino acid residues 50 to 70, a second transmembrane domain at amino acid residues 116 to 133, a first extracellular domain at amino acid residues 21 to 49 and a second extracellular domain at amino acid residues 134 to 140 of SEQ ID NO:136.

In one embodiment of a nucleotide sequence of human TANGO 383, the nucleotide at position 4 is cytosine (C). In this embodiment, the amino acid at position 2 is leucine (L). In an alternative embodiment, a species variant of human TANGO 383 has a nucleotide at position 4 which is adenine (A). In this embodiment, the amino acid at position 2 is isoleucine (I), i.e., a conservative substitution.

In one embodiment of a nucleotide sequence of human TANGO 383, the nucleotide at position 8 is guanine (G). In this embodiment, the amino acid at position 3 is serine (S).

In an alternative embodiment, a species variant of human TANGO 383 has a nucleotide at position 8 which is cytosine (C). In this embodiment, the amino acid at position 3 is threonine (T), i.e., a conservative substitution.

In one embodiment of a nucleotide sequence of human TANGO 383, the nucleotide at position 17 is adenine (A). In this embodiment, the amino acid at position 6 is lysine (K). In an alternative embodiment, a species variant of human TANGO 383 has a nucleotide at position 17 which is guanine (G). In this embodiment, the amino acid at position 6 is arginine (R), i.e., a conservative substitution.

In one embodiment of a nucleotide sequence of human TANGO 383, the nucleotide at position 57 is cytosine (C). In this embodiment, the amino acid at position 19 is aspartate (D). In an alternative embodiment, a species variant of human TANGO 383 has a nucleotide at position 57 which is guanine (G). In this embodiment, the amino acid at position 19 is glutamate (E), i.e., a conservative substitution.

One protein kinase C phosphorylation site is present in TANGO 383, and has the sequence SPR (at amino acids 21 to 24). TANGO 383 has one casein kinase II phosphorylation site which has the sequence SKAE (at amino acids 42 to 45). TANGO 383 has three N-myristylation sites. The first has the sequence GVELAS (at amino acids 24 to 29), the second has the sequence GAVLAH (at amino acids 84 to 89), and the third has the sequence GSSDSH (at amino acids 96 to 101). TANGO 383 has a consensus tyrosine phosphorylation site which has the amino acid sequence RGKREAGLY and (at amino acids 33 to 41). TANGO 383 also has an amidation site with the sequence RGKR (at amino acids 33-36).

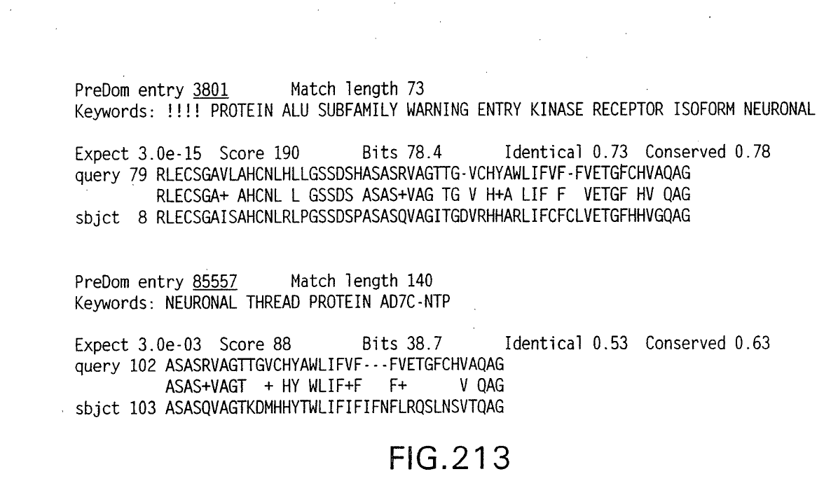

FIG. 213 depicts an alignment of the amino acid sequence of TANGO 383 and the amino acid sequence of Neuronal Thread Protein AD7C-NTP. The alignments demonstrates that the amino acid sequences of TANGO 383 and Neuronal Thread Protein AD7C-NTP are 52% identical. This alignment was performed using the ProDom NCBI-BLASTP2 program with graphical output using the following settings: Matrix: BLOSUM62; Expect: 0.1; Filter: none.

Thus, TANGO 383 exhibits homology to neural thread proteins which are phospho-proteins expressed in the central nervous system which are phosphorylated during neuritic sprouting. Therefore, TANGO 383 nucleic acids, proteins and modulators thereof may be used to diagnose disorders and/or inhibit or modulate neurodegenerative sprouting and synaptic disassociation associated with, e.g., Alzheimer's disease, and other diseases in neural tissue as discussed below.

Clone EpT383, which encodes human TANGO 383, was deposited with the American Type Culture Collection (10801 University Boulevard, Manassas, Va. 20110-2209) on Jun. 29, 1999 and assigned Accession Number PTA-295. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 383 Nucleic Acids, Polypeptides, and Modulators Thereof

As TANGO 383 was originally found in a prostate epithelium library, TANGO 383 nucleic acids, proteins, and modulators thereof can be used to diagnose disorders and/or modulate the proliferation, differentiation, and/or function of prostate cells. TANGO 383 nucleic acids, proteins and modulators thereof can be utilized to modulate processes involved in prostate development, differentiation and activity, including, but not limited to development, and differentiation and activation of prostate tissues and cells as well as any function associated with such cells, and amelioration of one or more symptoms associated with abnormal function of such cell types or disorders associated with such cell types. Such disorders can include, but are not limited to, malignant or benign prostate cell growth or inflammatory disorders (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, atypical prostatic stromal lesions).

TANGO 383 exhibits homology to neural thread proteins which are phospho-proteins expressed in the central nervous system which are phosphorylated during neuritic sprouting. Therefore, TANGO 383 nucleic acids, proteins and modulators thereof may be used to diagnose disorders and/or inhibit or modulate neurodegenerative sprouting and synaptic disassociation associated with, e.g., Alzheimer's disease. TANGO 383 nucleic acids, proteins and modulators thereof may also be utilized to diminish the effects of stroke and other neural damage, e.g., spinal cord injuries, infarction, infection, malignancy, exposure to toxic agents, nutritional deficiency, paraneoplastic syndromes, and degenerative nerve diseases including but not limited to Alzheimer's disease, Parkinson's disease, Huntington's Chorea, amyotrophic lateral sclerosis, progressive supra-nuclear palsy, and other dementias.

As TANGO 383 is a transmembrane protein, TANGO 383 nucleic acids, proteins and modulators thereof can be utilized to modulate intercellular signaling pathways, for example by disrupting ligand-receptor interactions or cellular interactions with the extra-cellular matrix.

As TANGO 383 is a transmembrane protein and thus likely a signaling molecule, TANGO 383 nucleic acids, proteins or modulators thereof, activities can include, e.g., (1) the ability to modulate, e.g., stabilize, promote, inhibit or disrupt, protein-protein interactions (e.g., homophilic and/or heterophilic), and protein-ligand interactions, e.g., in receptor-ligand recognition; (2) ability to modulate cell-cell interactions; (3) the ability to modulate the proliferation, differentiation and/or activity of neural cells; and (4) the ability to modulate intracellular signaling cascades (e.g., signal transduction cascades).

TANGO 383 expression can be utilized as a marker (e.g., an in situ marker) for specific tissues (e.g., the prostate) and/or cells (e.g., epithelial cells) in which TANGO 383 is expressed. TANGO 383 nucleic acids can also be utilized for chromosomal mapping, or as chromosomal markers, e.g., in radiation hybrid mapping.

### Human MANGO 346

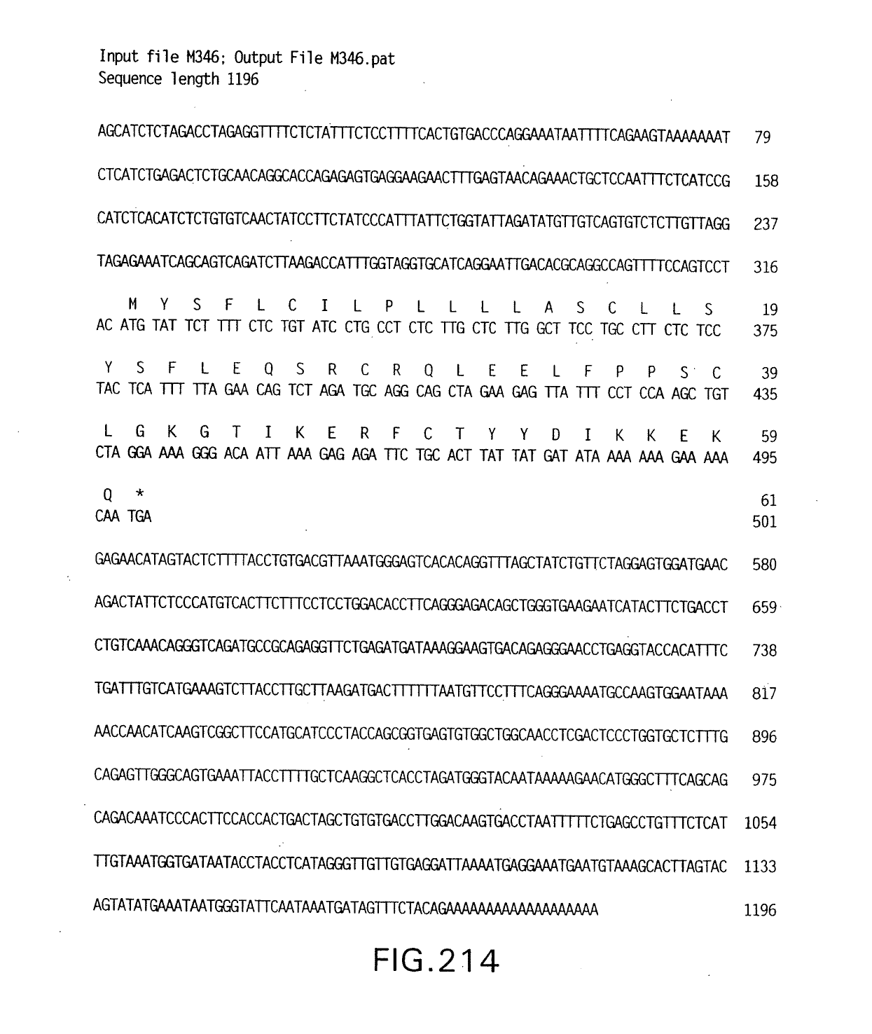

A MANGO 346 cDNA was identified from clones present in a human brain library among sequences that encode signal peptides. This analysis led to the identification of a clone, jlhbabS75g04, encoding full-length human MANGO 346. The human MANGO 346 cDNA of this clone is 1196 nucleotides long (FIG. 214; SEQ ID NO:138). The open reading frame of this cDNA (nucleotides 319 to 498 of SEQ ID NO: 137) encodes a 60 amino acid secreted protein (SEQ ID NO:138).

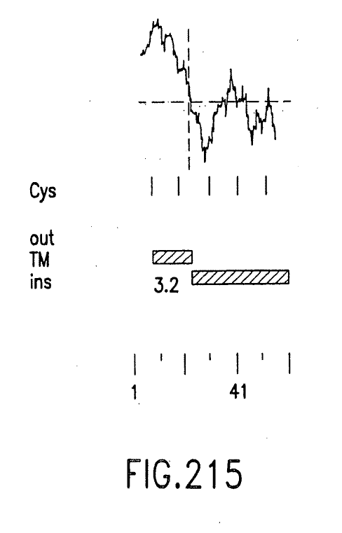

FIG. 215 depicts a hydropathy plot of human MANGO 346. The dashed vertical line separates the signal sequence (amino acids 1 to 19 of SEQ ID NO:138) on the left from the mature protein (amino acids 20 to 60 of SEQ ID NO: 138) on the right.

The signal peptide prediction program SIGNALP (Nielsen et al., 1997, Protein Engineering 10:1-6) predicted that human MANGO 346 includes a 19 amino acid signal peptide (amino acid 1 to amino acid 19 of SEQ ID NO: 138) preceding the mature human protein (corresponding to amino acid 20 to amino acid 60 of SEQ ID NO:138). The molecular weight of protein without post-translational modifications is 7.1 kDa prior to the cleavage of the signal peptide, 5.0 kDa after cleavage of the signal peptide.

In one embodiment of a nucleotide sequence of human MANGO 346, the nucleotide at position 13 is cytosine (C). In this embodiment, the amino acid at position 5 is leucine (L). In an alternative embodiment, a species variant of human MANGO 346 has a nucleotide at position 13 which is adenine (A). In this embodiment, the amino acid at position 5 is isoleucine (I), i.e., a conservative substitution.

In one embodiment of a nucleotide sequence of human MANGO 346, the nucleotide at position 59 is adenine (A). In this embodiment, the amino acid at position 20 is tyrosine (Y). In an alternative embodiment, a species variant of human MANGO 346 has a nucleotide at position 59 which is thymidine (T). In this embodiment, the amino acid at position 20 is phenylalanine (F), i.e., a conservative substitution.

In one embodiment of a nucleotide sequence of human MANGO 346, the nucleotide at position 61 is thymidine (T). In this embodiment, the amino acid at position 21 is serine (S). In an alternative embodiment, a species variant of human MANGO 346 has a nucleotide at position 61 which is adenine (A). In this embodiment, the amino acid at position 21 is threonine (T), i.e., a conservative substitution.

In one embodiment of a nucleotide sequence of human MANGO 346, the nucleotide at position 80 is guanine (G). In this embodiment, the amino acid at position 27 is arginine (R). In an alternative embodiment, a species variant of human MANGO 346 has a nucleotide at position 80 which is adenine (A). In this embodiment, the amino acid at position 27 is lysine (K), i.e., a conservative substitution.

One protein kinase C phosphorylation site is present in human MANGO 346 which has the sequence, TIK (at amino acids 44 to 46). Human MANGO 346 has three Casein Kinase II phosphorylation sites. The first has the sequence SFLE (at amino acids 21 to 24), the second has the sequence TIKE (at amino acids 44 to 47) and the third has the sequence TYYD (at amino acids 51 to 54). Human MANGO 346 has one prokaryotic membrane lipoprotein lipid attachment site. The sequence is CILPLLLLASC (at amino acids 6 to 16).

Clone EpM346, which encodes human MANGO 346, was deposited with the American Type Culture Collection (10801 University Boulevard, Manassas, Va. 20110-2209) on Jun. 29, 1999 and assigned Accession Number PTA-291. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of MANGO 346 Nucleic Acids, Polypeptides, and Modulators Thereof

As MANGO 346 was originally found in a human brain library, nucleic acids, proteins, and modulators thereof can be used to diagnose or identify disorders and/or modulate the proliferation, development, differentiation, and/or function of neural organs, e.g., neural tissues and cells, e.g., cells of the central nervous system, e.g., cells of the peripheral nervous system. MANGO 346 nucleic acids, proteins, and modulators thereof can also be used to diagnose or identify disorders and/or modulate symptoms associated with abnormal neural signaling and function, e.g., epilepsy, spinal cord injuries, infarction, infection, malignancy, exposure to toxic agents, nutritional deficiency, paraneoplastic syndromes, and degenerative nerve diseases including but not limited to Alzheimer's disease, Parkinson's disease, Huntington's Chorea, amyotrophic lateral sclerosis, progressive supra-nuclear palsy, and other dementias.

MANGO 346 nucleic acids, proteins and modulators thereof can, in addition to the above, be utilized to diagnose disorders, regulate or modulate development and/or differentiation of processes involved in central or peripheral nervous system formation and activity, as well as in ameliorating any symptom associated with a disorder of such cell types, tissues and organs.

MANGO 346 nucleic acids, proteins and modulators thereof can, in addition to the above, be utilized to regulate or diagnose disorders, modulate development and/or differentiation of processes involved in central or peripheral nervous system formation and activity, as well as in ameliorating any symptom associated with a disorder of such cell types, tissues and organs. Furthermore, the TANGO 346 proteins can be used to disrupt protein interaction or cellular signaling in brain tissues or cells. In particular, TANGO 346 proteins are useful to treat neural related disorders or neural damage, such as for regenerative neural repair after damage by trauma, degeneration, or inflammation e.g., spinal cord injuries, infarction, infection, malignancy, exposure to toxic agents, nutritional deficiency, paraneoplastic syndromes, and degenerative nerve diseases including but not limited to Alzheimer's disease, Parkinson's disease, Huntington's Chorea, amyotrophic lateral sclerosis, progressive supra-nuclear palsy, and other dementias.

As MANGO 346 is a secreted protein and thus likely a signaling molecule, MANGO 346 nucleic acids, proteins or modulators thereof, can be used to modulate MANGO 346 biological activities, which include, e.g., (1) the ability to modulate, e.g., stabilize, promote, inhibit or disrupt, protein-protein interactions (e.g., homophilic and/or heterophilic), and protein-ligand interactions, e.g., in receptor-ligand recognition; (2) ability to modulate cell-cell interactions; (3) the ability to modulate the proliferation, differentiation and/or activity of neural cells; and (4) the ability to modulate intracellular signaling cascades (e.g., signal transduction cascades).

MANGO 346 expression can be utilized as a marker (e.g., an in situ marker) for specific tissues (e.g., the brain) and/or cells (e.g., neurons) in which MANGO 346 is expressed. MANGO 346 nucleic acids can also be utilized for chromosomal mapping, or as chromosomal markers, e.g., in radiation hybrid mapping.

### Human MANGO 349

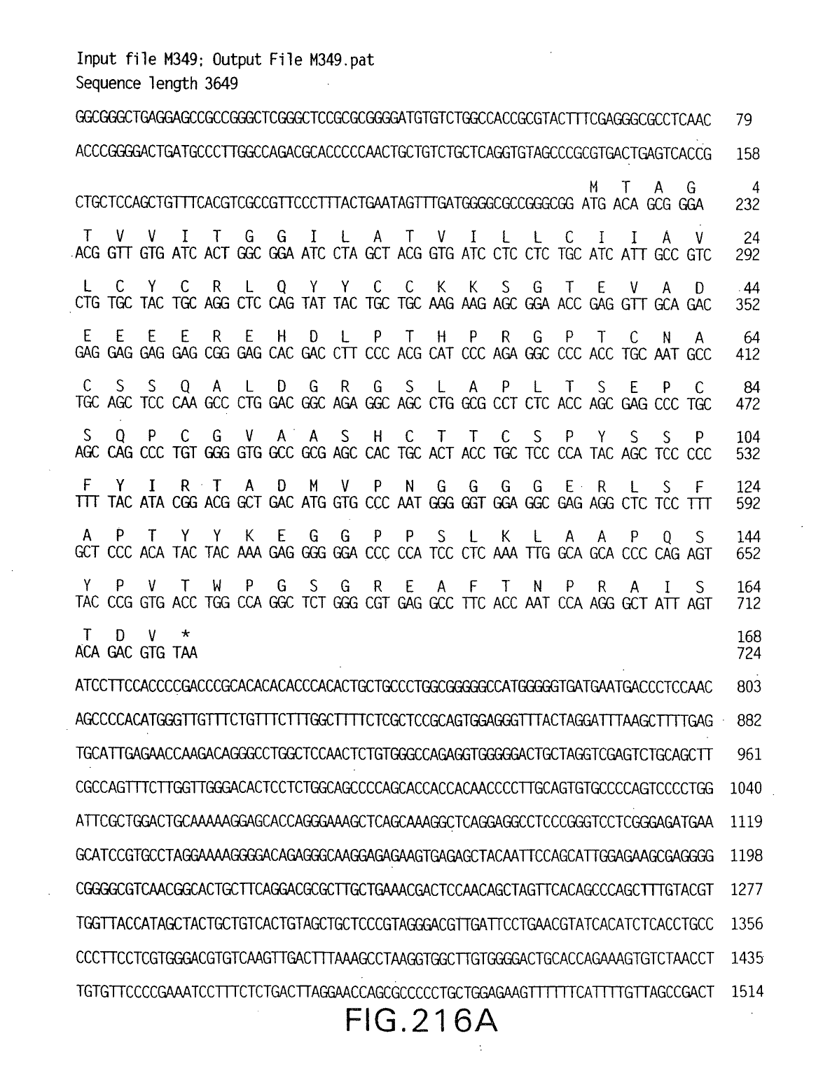

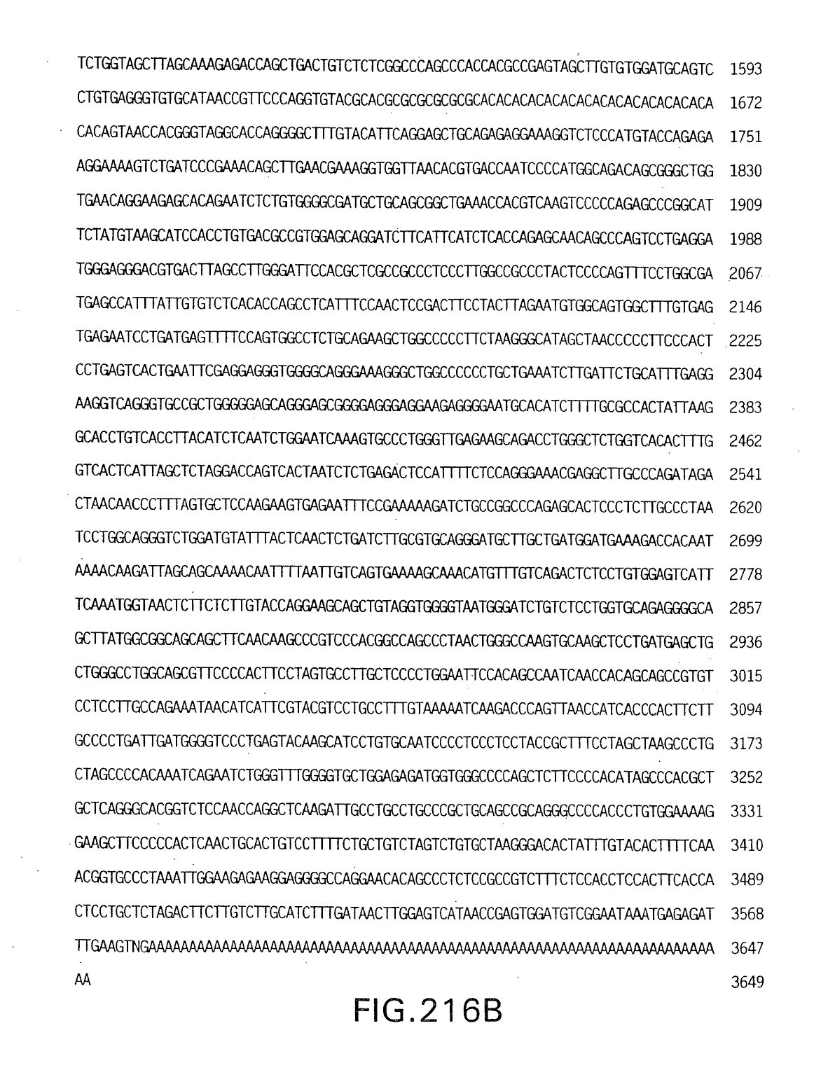

A cDNA encoding human MANGO 349 was identified by analyzing the sequences of clones present in a human brain library for sequences that encode wholly secreted or transmembrane proteins. This analysis led to the identification of a clone, jlhbae318gd08, encoding full-length human MANGO 349. The human cDNA of this clone is 3649 nucleotides long (FIG. 216A-216B; SEQ ID NO:139). The open reading frame of this cDNA (nucleotides 221 to 7218 of SEQ ID NO:139) encodes a 167 amino acid secreted protein (SEQ ID NO: 140).

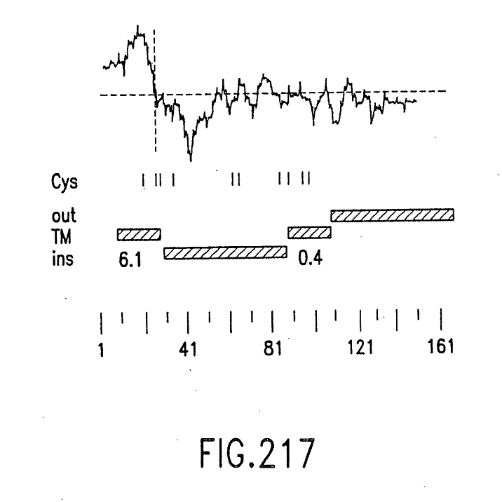

FIG. 217 depicts a hydropathy plot of human MANGO 349.

The signal peptide prediction program SIGNALP (Nielsen et al., 1997, Protein Engineering 10:1-6) predicted that human MANGO 349 includes a 26 amino acid signal peptide (amino acid 1 to amino acid 26) preceding the mature human protein (corresponding to amino acid 27 to amino acid 167). The molecular weight of human protein without post-translational modifications is 17.6 kDa prior to the cleavage of the signal peptide, 15.1 kDa after cleavage of the signal peptide.

In one embodiment of a nucleotide sequence of human MANGO 349, the nucleotide at position 4 is adenine (A). In this embodiment, the amino acid at position 2 is threonine (T). In an alternative embodiment, a species variant of human MANGO 349 has a nucleotide at position 4 which is thymine (T). In this embodiment, the amino acid at position 2 is serine (S), i.e., a conservative substitution.

In one embodiment of a nucleotide sequence of human MANGO 349, the nucleotide at position 61 is adenine (A). In this embodiment, the amino acid at position 21 is isoleucine (I). In an alternative embodiment, a species variant of human MANGO 349 has a nucleotide at position 61 which is cytosine (C). In this embodiment, the amino acid at position 21 is leucine (L), i.e., a conservative substitution.

In one embodiment of a nucleotide sequence of human MANGO 349, the nucleotide at position 86 is guanine (G). In this embodiment, the amino acid at position 29 is arginine, (R). In an alternative embodiment, a species variant of human MANGO 349 has a nucleotide at position 86 which is adenine (A). In this embodiment, the amino acid at position 29 is lysine (K), i.e., a conservative substitution.

In one embodiment of a nucleotide sequence of human MANGO 349, the nucleotide at position 123 is guanine (G). In this embodiment, the amino acid at position 41 is glutamate (E). In an alternative embodiment, a species variant of human MANGO 349 has a nucleotide at position 123 which is cytosine (C). In this embodiment, the amino acid at position 41 is aspartate (D), i.e., a conservative substitution.

Two Protein C Kinase phosphorylation sites are present in human MANGO 349. The first has the sequence SLK (at amino acids 136 to 1390) and the second has the sequence SGR (at atnino acids 152 to 1540). Two casein kinase II phosphorylation sites are present in human MANGO 349. The first has the sequence SGTE (at amino acids 38 to 5410), and the second has the sequence SGRE (at amino acids 152 to 1550). Human MANGO 349 has four N-myristylation sites. The first has the sequence GGILAT (at amino acids 10 to 150), the second has the sequence GTEVAD (at amino acids 39 to 440), the third has the sequence GVAASH (at amino acids 89 to 94), and the fourth has the sequence GGPPSL (at amino acids 132 to 1370).

Clone EpM349, which encodes human MANGO 349, was deposited with the American Type Culture Collection (10801 University Boulevard, Manassas, Va. 20110-2209) on Jun. 29, 1999 and assigned Accession Number PTA-295. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of MANGO 349 Nucleic Acids, Polypeptides, and Modulators Thereof

As MANGO 349 was originally found in a human brain library, nucleic acids, proteins, and modulators thereof can be used to diagnose or identify disorders and/or modulate the proliferation, development, differentiation, and/or function of neural organs, e.g., neural tissues and cells, e.g., cells of the central nervous system, e.g., cells of the peripheral nervous system. MANGO 349 nucleic acids, proteins, and modulators thereof can also be used to diagnose or identify disorders and/or modulate symptoms associated with abnormal neural signaling and function, e.g., epilepsy, stroke, traumatic injury, etc.

MANGO 349 nucleic acids, proteins and modulators thereof can, in addition to the above, be utilized to diagnose disorders, regulate or modulate development and/or differentiation of processes involved in central or peripheral nervous system formation and activity, as well as in ameliorating any symptom associated with a disorder of such cell types, tissues and organs. Furthermore, the TANGO 349 proteins can be used to disrupt protein interaction or cellular signaling in brain tissues or cells. In particular, TANGO 349 proteins could be useful to treat neural related disorders or neural damage, such as for regenerative neural repair after damage by trauma, degeneration, or inflammation e.g., spinal cord injuries, infarction, infection, malignancy, exposure to toxic agents, nutritional deficiency, paraneoplastic syndromes, and degenerative nerve diseases including but not limited to Alzheimer's disease, Parkinson's disease, Huntington's Chorea, amyotrophic lateral sclerosis, progressive supra-nuclear palsy, and other dementias.

As MANGO 349 is a secreted protein and thus likely a signaling molecular, MANGO 349 nucleic acids, proteins and modulators thereof can be used to diagnose disorders and/or modulate MANGO 349 biological activities, which include, e.g., (1) the ability to modulate, e.g., stabilize, promote, inhibit or disrupt, protein-protein interactions (e.g., homophilic and/or heterophilic), and protein-ligand interactions, e.g. in receptor-ligand recognition; (2) ability to modulate cell-cell interactions; (3) the ability to modulate proliferation, differentiation and/or activity of neural cells; and (4) the ability to modulate intracellular signaling cascades (e.g. signal transduction cascades).

MANGO 349 expression can be utilized as a marker (e.g., an in situ marker) for specific tissues (e.g., the brain) and/or cells (e.g., neurons) in which MANGO 349 is expressed. MANGO 349 nucleic acids can also be utilized for chromosomal mapping, or as chromosomal markers, e.g., in radiation hybrid mapping.

### INTERCEPT 307, MANGO 51, TANGO 361 and TANGO 499

The INTERCEPT 307, MANGO 511, TANGO 361 and TANGO 499 proteins and nucleic acid molecules comprise families of molecules having certain conserved structural and functional features.

For example, the INTERCEPT 307, MANGO 511, TANGO 361 and TANGO 499 proteins of the invention can have signal sequences.

In one embodiment, an INTERCEPT 307 protein can contain a signal sequence of about amino acids 1 to 23 of SEQ ID NO: 142.

In another embodiment, a MANGO 511 protein can contain a signal sequence of about 1 to 41 of SEQ ID NO: 144.

In another embodiment, a TANGO 361 protein can contain a signal sequence of about amino acids 1 to 35 of SEQ ID NO:146.

In another embodiment, a TANGO 499 form 1, variant 1 protein can contain a signal sequence of about amino acids 1 to 30 of SEQ ID NO: 148.

In another embodiment, a TANGO 499 form 2, variant 3 protein can contain a signal sequence of about amino acids 1 to 30 of SEQ ID NO: 150.

An INTERCEPT 307 family member can include one or more of the following domains: (1) an extracellular domain; (2) a transmembrane domain; and (3) a cytoplasmic domain. In one embodiment, an INTERCEPT 307 protein contains extracellular domains at about amino acid residues 24 to 153, 211 to 228 and 319 to 330, transmembrane domains at about amino acid residues 154 to 175, 192 to 210, 229 to 252, 296 to 318 and 331 to 348 and cytoplasmic domains at about amino acid residues 176 to 191, 253 to 295 and 349 to 362. In this embodiment, the mature INTERCEPT 307 protein corresponds to amino acids 24 to 362 of SEQ ID NO: 142.

An INTERCEPT 307 family member can include a signal sequence. In certain embodiments, a INTERCEPT 307 family member has the amino acid sequence, and the signal sequence is located at amino acids 1 to 21, 1 to 22, 1 to 23, 1 to 24 or 1 to 25. In such embodiments of the invention, the domains and the mature protein resulting from cleavage of such signal peptides are also included herein. For example, the cleavage of a signal sequence consisting of amino acids 1 to 23 results in an extracellular domain consisting of amino acids 24 to 153 and the mature INTERCEPT 307 protein corresponding to amino acids 24 to 362 of SEQ ID NO:142.

An INTERCEPT 307 family member can include one or more Gas vesicle protein GVPc-like domains. A gas vesicle protein GPVc-like domain as described herein can have the following-consensus sequence: F-L-Xaa-Xaa-Xaa-Xaa-Xaa-Xaa-Xaa-Xaa-A-Xaa-Q-Xaa-Xaa-Xaa-L-Xaa-Xaa-F, wherein F is phenylalanine, L is leucine, “Xaa” is any amino acid, A is alanine, and Q is glutamine.

In one embodiment, an INTERCEPT 307 family member has the amino acid sequence and, preferably, a gas vesicle protein GPVc-like domain is located at about amino acid positions 112 to 141. In another embodiment, an INTERCEPT 307 family member has the amino acid sequence and, preferably, a gas vesicle protein GPVc-like consensus sequence is located at about amino acid positions 122 to 141. In another embodiment, an INTERCEPT 307 family member includes one or more gas vesicle protein GPVc-like consensus sequences having an amino acid sequence that is at least about 55%, preferably at least about 65%, more preferably at least 75%, yet more preferably at least about 85%, and most preferably at least about 95% identical to amino acids 112 to 141. In yet another embodiment, an INTERCEPT 307 family member includes one or more gas vesicle protein GPVc-like domains having an amino acid sequence that is at least about 55%, preferably at least about 65%, more preferably at least 75%, yet more preferably at least about 85%, and most preferably at least about 95% identical to amino acids 122 to 141 of SEQ ID NO: 142.

In another embodiment an INTERCEPT 307 family member includes one or more gas vesicle protein GPVc-like domains having an amino acid sequence that is at least about 55%, preferably at least about 65%, more preferably at least 75%, yet more preferably at least about 85%, and most preferably at least about 95% identical to amino acids 112 to 141, and has at least one INTERCEPT 307 biological activity as described herein. In yet another embodiment, an INTERCEPT 307 family member includes one or more gas vesicle protein GPVc-like domain consensus sequences having an amino acid sequence that is at least about 55%, preferably at least about 65%, more preferably at least 75%, yet more preferably at least about 85%, and most preferably at least about 95% identical to amino acids 122 to 141, and has at least one INTERCEPT 307 biological activity as described herein.

In another embodiment, the gas vesicle protein GVPc-like domain of INTERCEPT 5307 is a gas vesicle protein GVPc domain. A gas vesicle protein GVPc domain typically has the following consensus sequence: F-L-Xaa-Xaa-T-Xaa-Xaa-Xaa-R-Xaa-Xaa-Xaa-A-Xaa-Xaa-Q-Xaa-Xaa-Xaa-L-Xaa-Xaa-F, wherein F is phenylalanine, L is leucine, “Xaa” is any amino acid, T is threonine, R is arginine, A is alanine and Q is glutamine. Gas vesicle protein GVPc domains are found in cyanobacterial and Archaebacteria microorganisms. Gas vesicles are small, hollow, gas filled protein structures that enable the bacteria to position themselves at a favorable depth in a liquid medium for growth. In this embodiment, an INTERCEPT 307 family member includes one or more gas vesicle protein GVPc domains having an amino acid sequence that is at least about 55%, preferably at least about 65%, more preferably at least 75%, yet more preferably at least about 85%, and most preferably at least about 95% identical to amino acids 112 to 141 of SEQ ID NO: 142.

A MANGO 511 family member can include one or more of the following domains: (1) an extracellular domain; (2) a transmembrane domain; and (3) a cytoplasmic domain. In one embodiment, a MANGO 511 protein contains an extracellular domain at about amino acid residues 42 to 265, a transmembrane domain at about amino acid residues 266 to 284, and a cytoplasmic domain at about amino acid residues 285 to 299. In this embodiment, the mature MANGO 511 protein corresponds to amino acids 42 to 299 of SEQ ID NO: 144.

A MANGO 511 family member can include a signal sequence. In certain embodiments, a MANGO 511 family member has the amino acid sequence, and the signal sequence is located at amino acids 1 to 39, 1 to 40, 1 to 41, 1 to 42 or 1 to 43. In such embodiments of the invention, the domains and the mature protein resulting from cleavage of such signal peptides are also included herein. For example, the cleavage of a signal sequence consisting of amino acids 1 to 41 results in an extracellular domain consisting of amino acids 42 to 265 and a mature MANGO 511 protein corresponding to amino acids 42 to 299 of SEQ ID NO:144.

A MANGO 511 family member can include one or more Ig-like domains. A MANGO 511 Ig-like domain as described herein has the following consensus sequence, beginning about 1 to 15 amino acid residues, more preferably about 3 to 10 amino acid residues, and most preferably about 5 amino acid residues from the domain C-terminus: [FY]-Xaa-C, wherein [FY] is either a phenylalanine or a tyrosine residue (preferably tyrosine), where “Xaa” is any amino acid, and C is a cysteine residue. In one embodiment, a MANGO 511 family member includes one or more Ig-like domains having an amino acid sequence that is at least about 55%, preferably at least about 65%, more preferably at least 75%, yet more preferably at least about 85%, and most preferably at least about 95% identical to amino acids 60 to 118 of SEQ ID NO: 144.

In another embodiment, a MANGO 511 family member includes one or more MANGO 511 Ig-like domains having an amino acid sequence that is at least about 55%, preferably at least about 65%, more preferably at least about 75%, yet more preferably at least about 85%, and most preferably at least about 95% identical to amino acids 60 to 118, and has a conserved cysteine residue about 8 residues downstream from the N-terminus of the Ig-like domain. In another embodiment, a MANGO 511 family member includes one or more Ig-like domains having an amino acid sequence that is at least 55%, preferably at least about 65%, more preferably at least about 75%, yet more preferably at least about 85%, and most preferably at least about 95% identical to amino acids 60 to 118, has a conserved cysteine residue about 8 residues downstream from the N-terminus of the Ig-like domain, and has a conserved cysteine within the consensus sequence that forms a disulfide with said first conserved cysteine.

In yet another embodiment, a MANGO 511 family member includes one or more MANGO 511 Ig-like domains having an amino acid sequence that is at least 55%, preferably at least about 65%, more preferably at least about 75%, yet more preferably at least about 85%, and most preferably at least about 95% identical to amino acids 60 to 118, and has a conserved cysteine residue about 8 residues downstream from the N-terminus of the Ig-like domain, has a conserved cysteine within the consensus sequence that forms a disulfide with said first conserved cysteine, and has at least one MANGO 511 biological activity as described herein.

In another embodiment, the Ig-like domain of MANGO 511 is an Ig domain. An Ig domain as used in the context of MANGO 511 has the following consensus sequence, beginning at about 1 to 15 amino acid residues, more preferably about 3 to 10 amino acid residues, and most preferably about 5 amino acid residues from the C-terminal end of the domain: [FY]-Xaa-C-Xaa-[VA]-COO—, wherein [FY] is either a phenylalanine or a tyro sine residue (preferably tyrosine), where “Xaa” is any amino acid, C is a cysteine residue, [VA] is either valine or an alanine residue (preferably alanine), and COO— is the C-terminus of the domain. In this embodiment, a MANGO 511 family member includes one or more Ig-like domains having an amino acid sequence that is at least about 55%, preferably at least about 65%, more preferably at least 75%, yet more preferably at least about 85%, and most preferably at least about 95% identical to amino acids 60 to 118 of SEQ ID NO: 144.

A TANGO 361 family member can include one or more of the following domains: (1) an extracellular domain; (2) a transmembrane domain; and (3) a cytoplasmic domain. In one embodiment, a TANGO 361 protein contains an extracellular domain at about amino acid residues 235 to 423 a transmembrane domain at about amino acid residues 217 to 234, and a cytoplasmic domains at about amino acid residues 36 to 216 of SEQ ID NO: 146. In this embodiment, the mature TANGO 361 protein corresponds to amino acids 36 to 423 of SEQ ID NO: 146.

A TANGO 361 family member can include a signal sequence. In certain embodiments, a TANGO 361 family member has the amino acid sequence, and the signal sequence is located at about amino acids 1 to 33, 1 to 34, 1 to 35, 1 to 36 or 1 to 37. In such embodiments of the invention, the domains and the mature protein resulting from cleavage of such signal peptides are also included herein. For example, the cleavage of a TANGO 361 signal sequence consisting of amino acids 1 to 35 results in an extracellular domain consisting of amino acids 235 to 423 and the mature TANGO 361 protein corresponding to amino acids 36 to 423 of SEQ ID NO: 146.

A TANGO 361 family member can include one or more SEA domains. As used herein, the term “SEA domain” refers to a protein domain that can be found in TANGO 361 proteins and can regulate protein-protein or protein binding to carbohydrate side chains. A SEA domain typically has about 50-200 amino acid residues, preferably about 75-150 amino acid residues, more preferably about 80-140 amino acid residues, and most preferably about 115-135 amino acid residues of SEQ ID NO: 146.

A SEA domain typically has the following consensus sequence: h-t-h-Xaa-h-Xaa-h-Xaa-Xaa-Xaa-Xaa-h-Xaa-a-t-t-t-h-t-t-t-Xaa-o-Xaa-Xaa-a-Xaa-Xaa-h-Xaa-t-t-H-Xaa-t-Xaa-H-Xaa-t-Xaa-a-t-t-Xaa-Xaa-Xaa-Xaa-Xaa-Xaa-Xaa-h-h-Xaa-Xaa-Xaa-Xaa-Xaa-Xaa-Xaa-Xaa-t-t-Xaa-Xaa-h-Xaa-Xaa-Xaa-h-Xaa-h-t-h-h-Xaa-t-Xaa-Xaa-Xaa-Xaa-Xaa-t-t-t-h-t-Xaa-Xaa-Xaa-Xaa-t-Xaa-h-t-Xaa-Xaa-Xaa-Xaa-Xaa-h-Xaa-Xaa-Xaa-t-Xaa-Xaa-Xaa-Xaa-t-Xaa-Xaa-t-h-Xaa-Xaa-Xaa-Xaa-t, wherein t is a glycine, proline or polar amino acid, h is a hydrophobic amino acid, a is an aromatic amino acid and o is a serine or threonine. These domains are predominantly found in adhesive proteins present in heavily glycosylated environments. For example, SEA domains are found in a 63 kDa sea urchin sperm protein, agrin, enterokinase, perlecan, the breast cancer marker MUC1 (episialin) and the cell surface antigen 114/A10 (Bork and Patthy, (1995) Prot. Sci. 4:1421-1425).

In one embodiment, a TANGO 361 family member has the amino acid sequence and, preferably, a SEA domain is located at about amino acids 47 to 170. In another embodiment, a TANGO 361 family member includes one or more SEA domains having an amino acid sequence that is at least about 55%, preferably at least about 65%, more preferably at least 75%, yet more preferably at least about 85%, and most preferably at least about 95% identical to amino acids 47 to 170 of SEQ ID NO:146.

In another embodiment, a TANGO 361 family member includes one or more SEA domains having an amino acid sequence that is at least about 55%, preferably at least about 65%, more preferably at least 75%, yet more preferably at least about 85%, and most preferably at least about 95% identical to amino acids 44 to 170 of SEQ ID NO: 146, and has at least one TANGO 361 biological activity as described herein.

A TANGO 361 family member can include a serine protease domain. Serine protease domains are typically found in serine proteases, and can be found in, among other proteins, e.g., blood coagulation factors VII, XI, and X, thrombin, plasminogen, tryptases, such as trypsin, airway trypsin-like proteases, mast cell proteases, and members of the complement system which are known for regulation of energy balance and suppression of infectious agents. As used herein, the term “serine protease domain” refers to a polypeptide sequence that includes about 100-400 amino acid residues, preferably about 150-350 amino acid residues, more preferably about 200-300 amino acid residues, and most preferably about 225-260 amino acid residues. A serine protease typically has two consensus sequences. The first consensus sequence is a histidine active site and has the following sequence: [LIVM]-[ST]-A-[STAG]-H-C, wherein [LIVM] is a leucine, isoleucine, valine, or methionine, [ST] is a serine or threonine, A is alanine, [STAG] is serine, threonine, alanine or glycine, H is histidine and C is cysteine. The second consensus sequence is a serine active site and has the following consensus sequence: [DNSTAGC]-[GSTAPIMVQH]-x(2)-G-[DE]-S-G-[GS]-[SAPHV]-[LIVMFYH]-[LIVMFYSTANQH], wherein [DNSTAGC] is an aspartic acid, asparagine, serine, threonine, alanine, glycine, or cysteine, [GSTAPIMVQH] is a glycine, serine, threonine, alanine, proline, isoleucine, methionine, valine, glutamine, or histidine, x(2) is two consecutive amino acids, G is a glycine, [DE] is an aspartic acid or glutamic acid, S is a serine, G is a glycine, [GS] is a glycine or serine, [SAPHV] is a serine, alanine, proline, histidine or valine, [LIVMFH] is a leucine, isoleucine, valine, methionine, phenylalanine, tyrosine, tryptophan or histidine, [LIVMFYSTANQH] is a leucine, isoleucine, valine, methionine, phenylalanine, tyrosine, serine, threonine, alanine, asparagine, glutamine or histidine.

In one embodiment, a TANGO 361 family member has the amino acid sequence in which a serine protease domain appears at about amino acids 192 to 417. Preferably, a histidine active site consensus sequence is located at about amino acids 228 to 233 and a serine active site consensus sequence is located at 367 to 378. In another embodiment, a TANGO 361 family member includes one or more serine protease domain consensus sequences having an amino acid sequence that is at least about 55%, preferably at least about 65%, more preferably at least 75%, yet more preferably at least about 85%, and most preferably at least about 95% identical to amino acids 228 to 233 or 367 to 378 of SEQ ID NO:146.

In another embodiment, a TANGO 361 family member includes one or more serine protease domain consensus sequences having an amino acid sequence that is at least about 55%, preferably at least about 65%, more preferably at least 75%, yet more preferably at least about 85%, and most preferably at least about 95% identical to amino acids 228 to 233 or 367 to 378 of SEQ ID NO: 146, and has at least one TANGO 361 biological activity as described herein.

A TANGO 499 family member can include one or more of the following domains: 1) a signal sequence; and 2) a secreted protein. In one embodiment, a TANGO 499 protein is a secreted protein containing a signal sequence of 1 to 30 amino acids and is an immature protein of 254 amino acids. In this embodiment, the mature TANGO 499 protein corresponds to amino acids 31 to 254 of SEQ ID NO:148. In certain embodiments, a TANGO 499 family member has the amino acid sequence, and contains a signal sequence that is preferably located at about amino acids 1 to 28, 1 to 29, 1 to 31 or 1 to 32. In such embodiments of the invention, the domains and the mature protein resulting from cleavage of such signal peptides are also included herein. For example, the cleavage of a signal sequence consisting of amino acids 1 to 31 results in a mature protein comprising a secreted protein of amino acids 32 to 254 of SEQ ID NO: 148.

In certain embodiments, a TANGO 499 family member has the amino acid sequence, and contains a signal sequence that is preferably located at about amino acids 1 to 28, 1 to 29, 1 to 31 or 1 to 32. In such embodiments of the invention, the domains and the mature protein resulting from cleavage of such signal peptides are also included herein. For example, the cleavage of a signal sequence consisting of amino acids 1 to 31 results in a mature protein comprising a secreted protein of amino acids 32 to 229 of SEQ ID NO: 148.

In one embodiment, a TANGO 499 family member is a polypeptide comprising the amino acid sequence of SEQ ID NO:148. In another embodiment, a TANGO 499 family member is a polypeptide comprising the amino acid sequence of SEQ ID NO: 150.

### Human Intercept 307

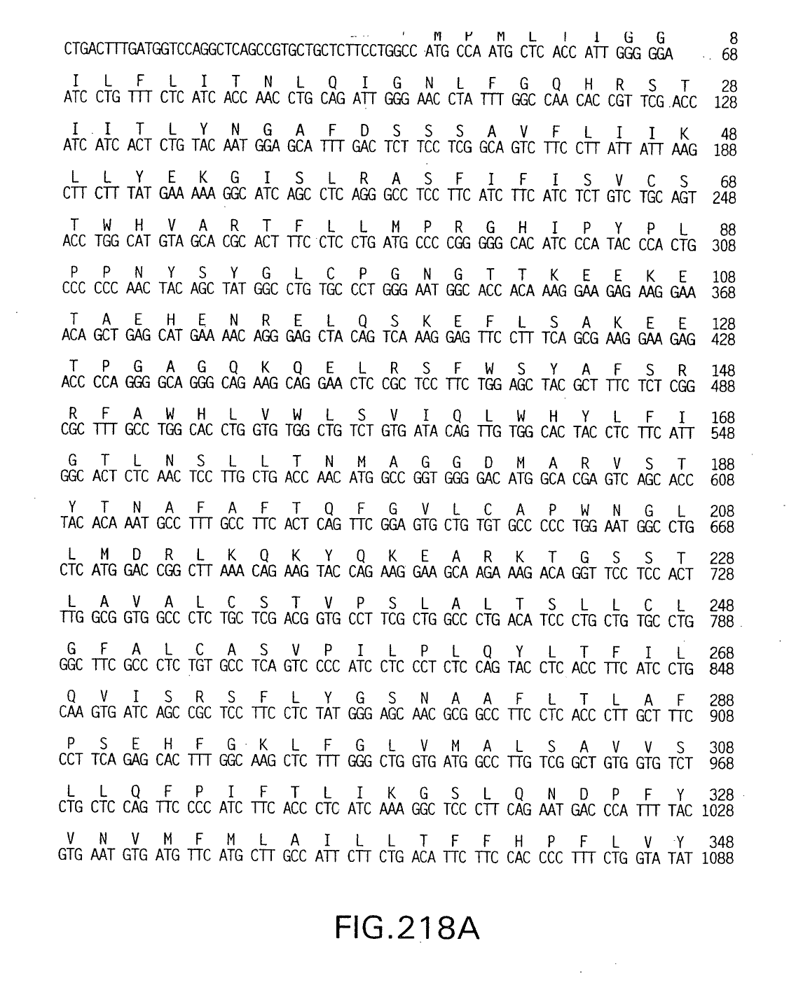

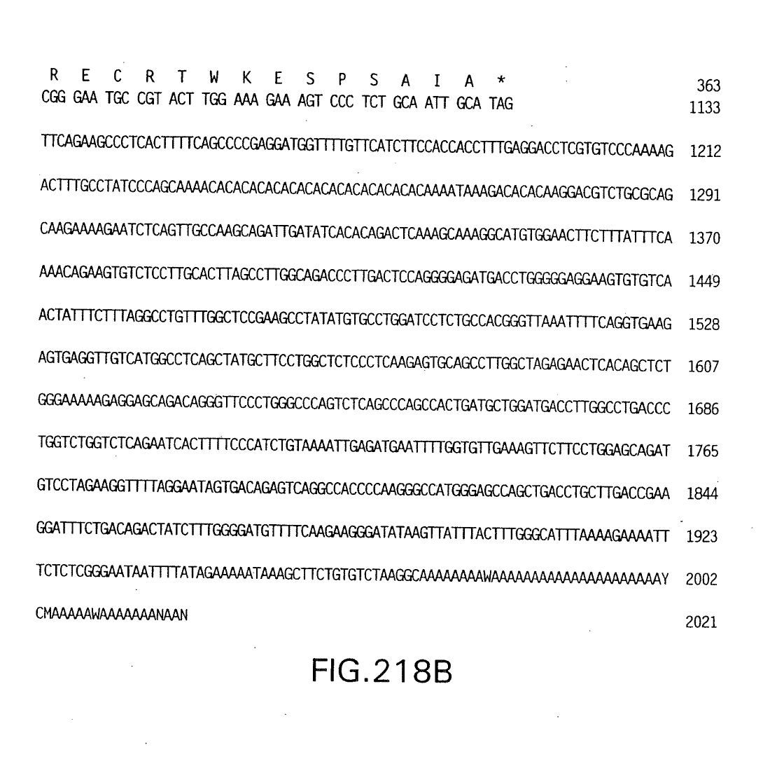

A cDNA encoding human INTERCEPT 307 was identified by analyzing the sequences of clones present in a human TH-2 induced T-cell library for sequences that encode wholly secreted or transmembrane proteins. This analysis led to the identification of a clone, jthtg033c10, encoding full-length human INTERCEPT 307. The human INTERCEPT 307 cDNA of this clone is 2021 nucleotides long (FIG. 218A-218B; SEQ ID NO:141). The open reading frame of this cDNA (nucleotides 45 to 1130 of SEQ ID NO:141) encodes a 362 amino acid transmembrane protein (SEQ ID NO:142)

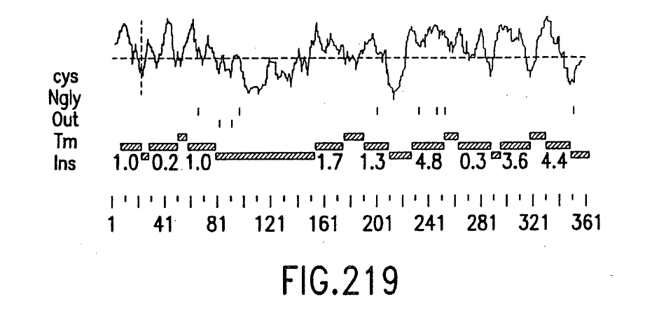

FIG. 219 depicts a hydropathy plot of human INTERCEPT 307.

The signal peptide prediction program SIGNALP (Nielsen et al., 1997, Protein Engineering 10:1-6) predicted that human INTERCEPT 307 includes a 23 amino acid signal peptide (amino acid 1 to amino acid 23 of SEQ ID NO: 142) preceding the mature INTERCEPT 307 protein (corresponding to amino acid 24 to amino acid 362 of SEQ ID NO: 142). In instances wherein the signal peptide is cleaved, the molecular weight of INTERCEPT 307 protein without post-translational modifications is 40.6 kDa prior to the cleavage of the signal peptide, and 38.1 kDa after cleavage of the signal peptide.

Human INTERCEPT 307 protein is a transmembrane protein that contains extracellular domains at amino acid residues 24 to 153, 211 to 228, and 319 to 330, transmembrane domains at amino acid residues 154 to 175, 192 to 210, 229 to 252, 296 to 319, and 331 to 348, and cytoplasmic domains at amino acid residues 176 to 191, 253 to 295, and 349 to 362 of SEQ ID NO: 142.

In instances wherein the signal peptide is not cleaved, a human INTERCEPT 307 protein is a transmembrane protein that contains extracellular domains at amino acid residues 1 to 153, 211 to 228, and 319 to 330, transmembrane domains at amino acid residues 154 to 175, 192 to 210, 229 to 252, 296 to 319, and 331 to 348, and cytoplasmic domains at amino acid residues 176 to 191, 253 to 295, and 349 to 362 of SEQ ID NO:142.

Alternatively, in another embodiment, a human INTERCEPT 307 protein contains cytoplasmic domains at amino acid residues 24 to 153, 211 to 228, and 319 to 330, transmembrane domains at amino acid residues 154 to 175, 192 to 210, 229 to 252, 296 to 319, and 331 to 348, and extracellular domains at amino acid residues 176 to 191, 253 to 295, and 349 to 362 of SEQ ID NO:142.

In one embodiment a cDNA sequence of human INTERCEPT 307 has a nucleotide at position 54 which is cytosine (C). In this embodiment, the cDNA contains an open reading frame encoding a polypeptide having an amino acid at position 4 that is leucine (L). In an alternative embodiment, a species variant cDNA sequence of human INTERCEPT 307 has a nucleotide at position 54 which is adenine (A). In this embodiment, the cDNA contains an open reading frame encoding a polypeptide having an amino acid at position 4 that is isoleucine (I), i.e., a conservative substitution.

In another embodiment a cDNA sequence of human INTERCEPT 307 has a nucleotide at position 76 which is thymine (T). In this embodiment, the cDNA contains an open reading frame encoding a polypeptide having an amino acid at position 11 that is phenylalanine (F). In an alternative embodiment, a species variant cDNA sequence of human INTERCEPT 307 has a nucleotide at position 76 which is adenine (A). In this embodiment, the cDNA contains an open reading frame encoding a polypeptide having an amino acid at position 11 that is tyrosine (Y), i.e., a conservative substitution.

In another embodiment a cDNA sequence of human INTERCEPT 307 has a nucleotide at position 87 which is adenine (A). In this embodiment, the cDNA contains an open reading frame encoding a polypeptide having an amino acid at position 15 that is asparagine (N). In an alternative embodiment, a species variant cDNA sequence of human INTERCEPT 307 has a nucleotide at position 87 which is guanine (G). In this embodiment, the cDNA contains an open reading frame encoding a polypeptide having an amino acid at position 15 that is aspartate (D), i.e., a conservative substitution.

In another embodiment a cDNA sequence of human INTERCEPT 307 has a nucleotide at position 123 which is thymidine (T). In this embodiment, the cDNA contains an open reading frame encoding a polypeptide having an amino acid at position 27 that is serine (S). In an alternative embodiment, a species variant cDNA sequence of human INTERCEPT 307 has a nucleotide at position 180 which is adenine (A). In this embodiment, the cDNA contains an open reading frame encoding a polypeptide having an amino acid at position 27 that is threonine (T), i.e., a conservative substitution.

Human INTERCEPT 307 includes a gas vesicle protein GVPc-like domain (at amino acids 112 to 141 of SEQ ID NO:142).

Two N-glycosylation sites are present in INTERCEPT 307. The first has the sequence NYSY (at amino acid residues 91 to 94) and second has the sequence NGTT (at amino acid residues 100 to 103). Five protein kinase C phosphorylation sites are present in INTERCEPT 307. The first has the sequence SLR (at amino acid residues 56 to 58), the second has the sequence TTK (at amino acid residues 102 to 104), the third has the sequence SAK (at amino acid residues 124 to 126), the fourth has the sequence SRR (at amino acid residues 147 to 149), and the fifth has the sequence TWK (at amino acid residues 353 to 355). INTERCEPT 307 has three casein kinase II phosphorylation sites. The first has the sequence TTKE (at amino acid residues 102 to 105), the second has the sequence SAKE (at amino acid residues 124 to 127), and the third has the sequence TWKE (at amino acid residues 353 to 356). Eight N-myristylation sites are present in INTERCEPT 307. The first has the sequence GNLFGQ (at amino acid residues 19 to 24), the second has the sequence GAFDSS (at amino acid residues 35 to 40), the third has the sequence GLCPGN (at amino acid residues 95 to 100), the fourth has the sequence GTLNSL (at amino acid residues 169 to 174), the fifth has the sequence GGDMAR (at amino acid residues 180 to 185), the sixth has the sequence GSNAAF (at amino acid residues 278 TO 283), the seventh has the sequence GLVMAL (at amino acid residues 298 to 303), and the eighth has the sequence GSLQND (at amino acid residues 320 to 325). INTERCEPT 307 has a leucine zipper pattern with the sequence LFGLVMALSAWSLLQFPIFTL at amino acid residues 296 to 317.

The INTERCEPT 307 gene maps to human chromosome 11 between markers D11S1357 and D11S1765.

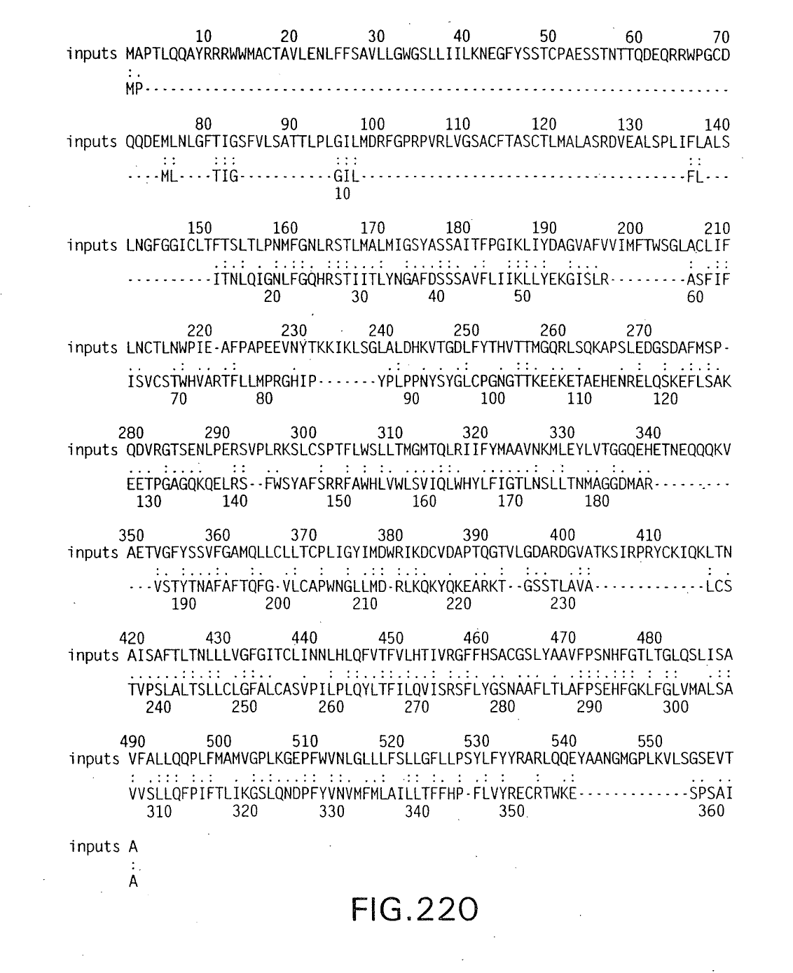

FIG. 220 shows an alignment of the human INTERCEPT 307 amino acid sequence with the prostate cancer gene PB39 amino acid sequence (Accession Number NM_{—}003627). The alignment shows that there is a 21.0% overall amino acid sequence identity between INTERCEPT 307 and PB39. PB39 is expressed in tissues of the adult colon, small intestine, ovary, prostate, spleen, skeletal muscle and pancreas. PB39 is also expressed in fetal kidney, liver and lung. The expression of PB39 has been shown to be increased early in prostate cancer development and PB39 can play a role in the development of human prostate cancer. As such, INTERCEPT 307, nucleic acids and proteins may be useful, for example, as early markers for the development of prostate cancer (e.g., early markers for the development of prostatic intraepithelial neoplasia (PIN)).

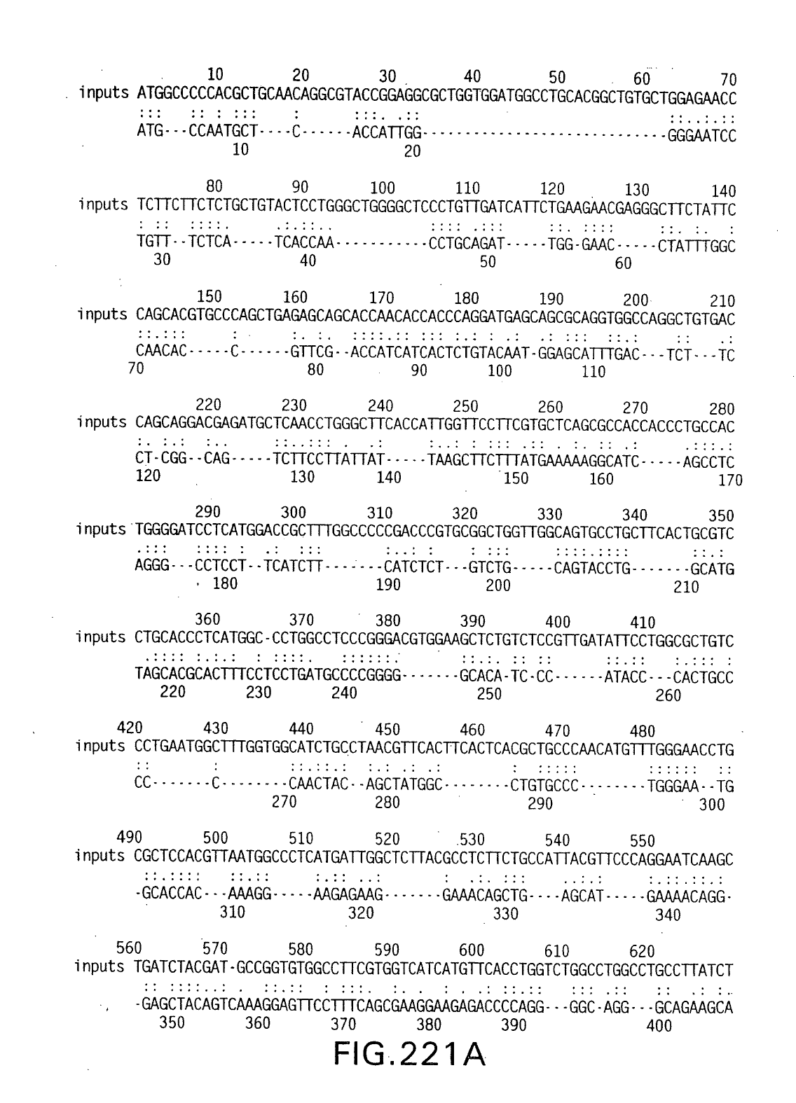

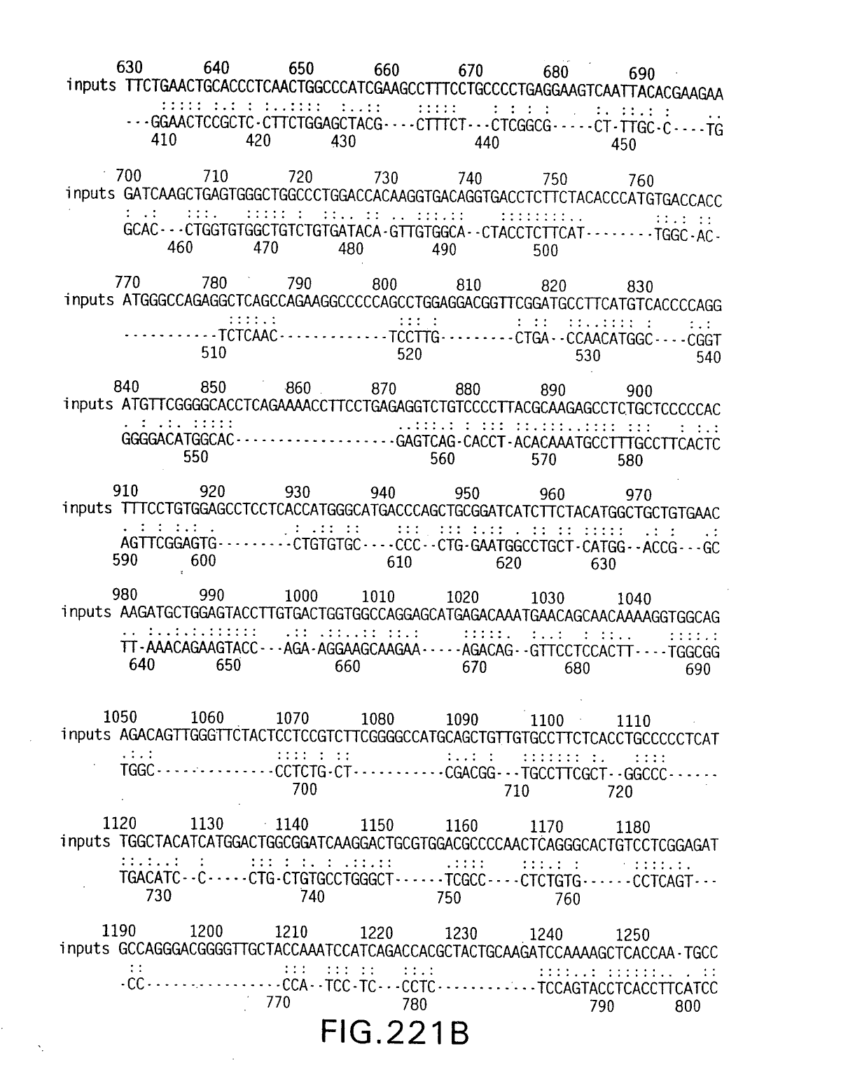

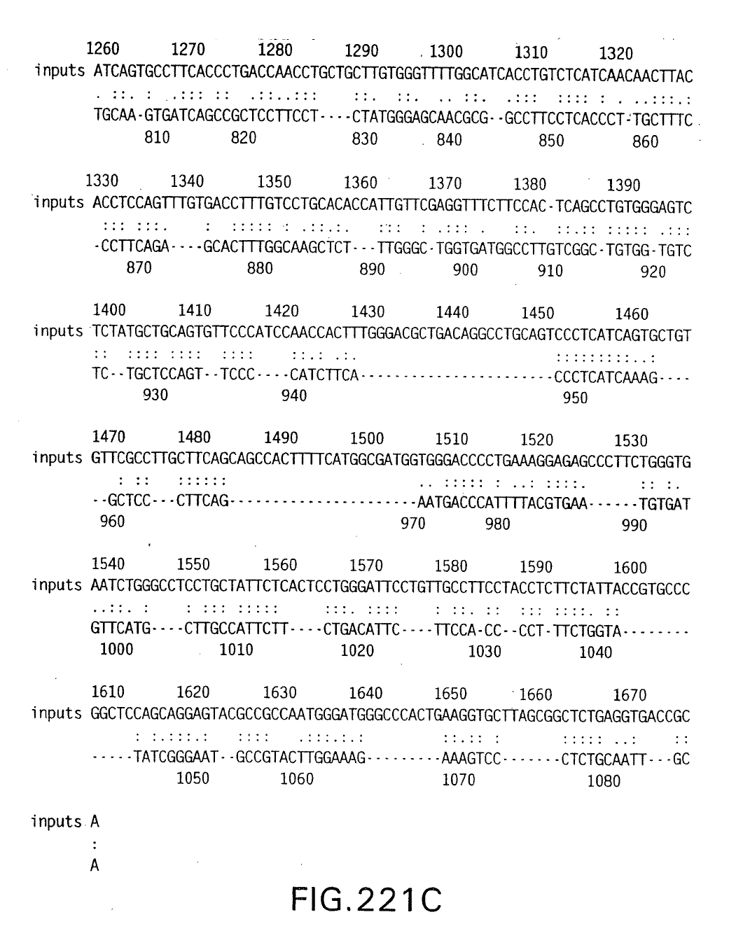

FIG. 221A-221C shows an alignment of the nucleotide sequence of INTERCEPT 307 coding region and the nucleotide sequence of PB39 coding region (Accession Number AF045584). The alignment shows a 40.9% overall sequence identity between the two nucleotide sequences. The full-length INTERCEPT 307 nucleic acid sequence and PB39 cDNA (Accession Number NM_{—}003627) have an overall sequence identity of 44.0%.

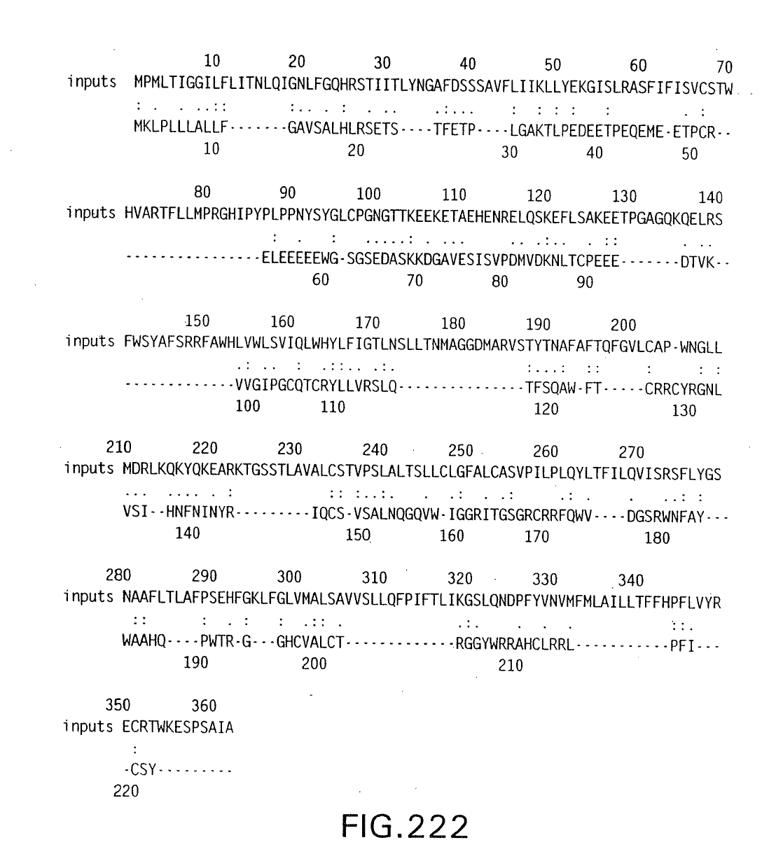

FIG. 222 shows an alignment of the human INTERCEPT 307 amino acid sequence with the human eosinophil granule major basic protein amino acid sequence (Accession Number Z26248). The alignment shows that there is a 13.8% overall amino acid sequence identity between INTERCEPT 307 and human eosinophil granule major basic protein. Human eosinophil granule major basic protein (MBP) is expressed in eosinophils and has toxic effects on many targets, including helminths, protozoa and bacteria and surrounding cells (Gleich, A. J. et al. (1993) Annu. Rev. Med. 44:85-101). MBP mediates damage to the respiratory epithelium (e.g., desquamation and destruction of sputum ciliated cells) in individuals with asthma, and increased MBP concentration has been shown to be a good marker for asthma (Frigas, E. et al. (1986) J. Allergy Clinical Immunol. 77:537-537).

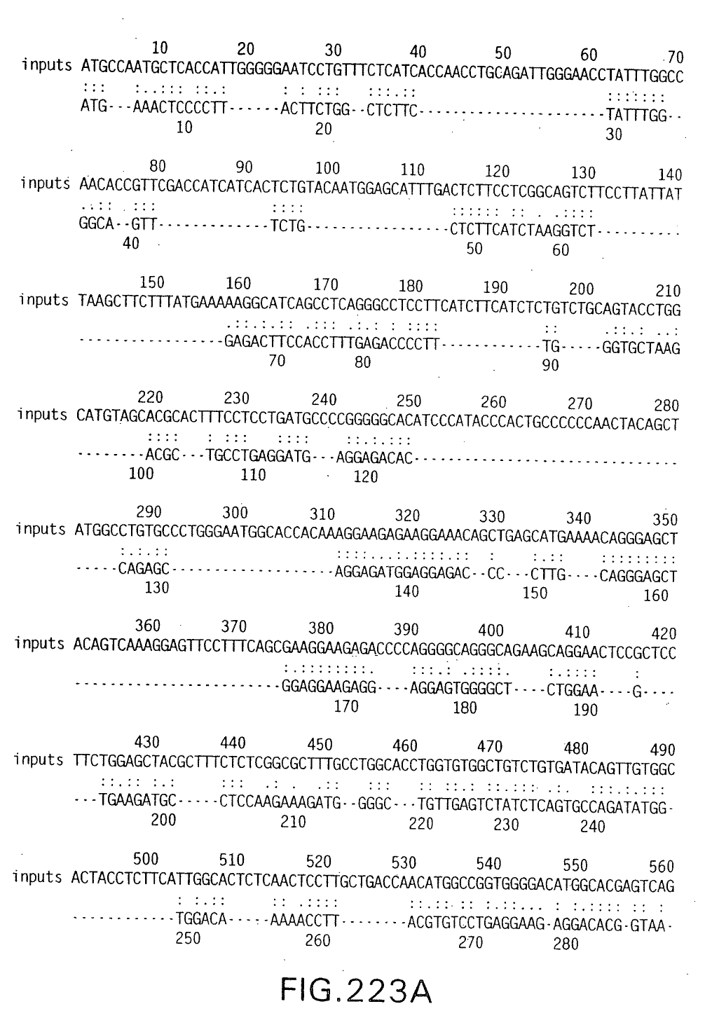

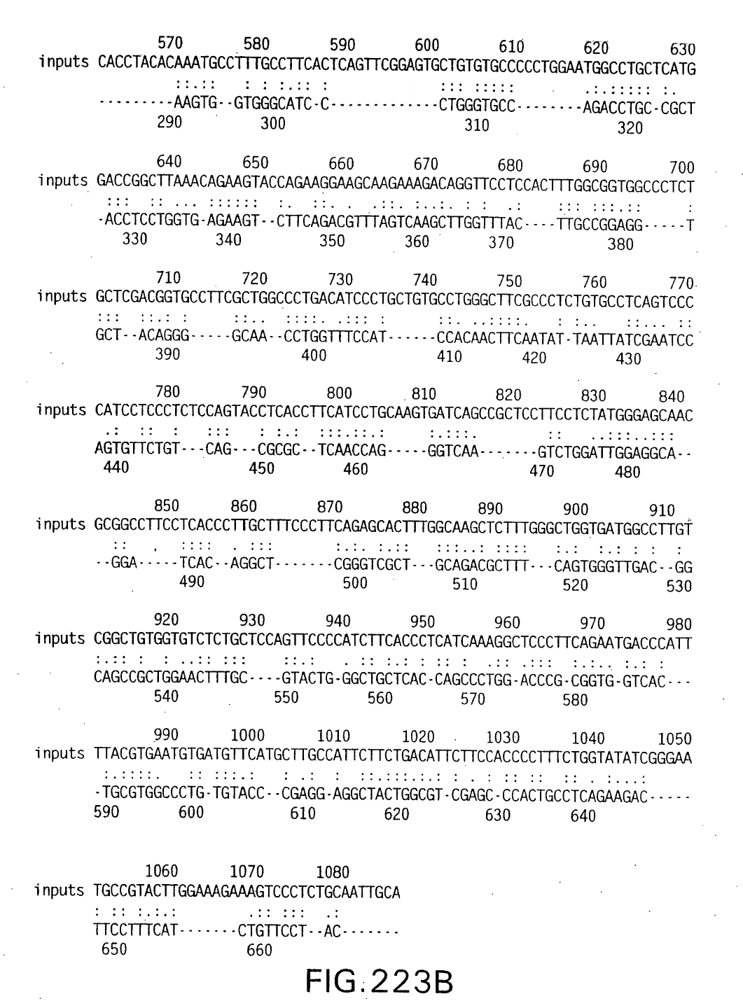

FIG. 223A-223B shows an alignment of the nucleotide sequence of INTERCEPT 307 coding region and the nucleotide sequence of human eosinophil granule major basic protein amino acid sequence coding region (Accession Number Z26248). The alignment shows a 38.1% overall sequence identity between the two nucleotide sequences. The full-length INTERCEPT 307 nucleic acid sequence and human eosinophil granule major basic protein cDNA (Accession Number Z26248) have an overall sequence identity of 57.3%.

Clone INT307, which encodes human INTERCEPT 307, was deposited with the American Type Culture Collection (10801 University Boulevard, Manassas, Va. 20110-2209) on Jul. 29, 1999 and assigned Accession Number PTA-455. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of INTERCEPT 307 Nucleic Acids, Polypeptides and Modulators Thereof

As INTERCEPT 307 was originally found in a human TH2-induced T-cell library, INTERCEPT 307 nucleic acids, proteins, and modulators thereof can be used to modulate the proliferation, development, differentiation, and/or function of lymphocytes, e.g., T-lymphocytes. INTERCEPT 307 nucleic acids, proteins and modulators thereof can be utilized to modulate immune-related processes, e.g., the host immune response by, for example, modulating the formation of and/or binding to immune complexes, detection and defense against surface antigens and bacteria, and immune surveillance for rapid removal or pathogens. Such INTERCEPT 307 compositions and modulators thereof can be utilized, e.g., to ameliorate incidence of any symptoms associated with disorders that involve such immune-related processes, including, but not limited to, viral or bacterial infection, and inflammatory disorders (e.g., bacterial or viral infection, psoriasis, allergies and inflammatory bowel diseases). INTERCEPT 307 nucleic acids, proteins and modulators thereof can be used to modulate or treat immune related disorders, e.g., immunodeficiency disorders (e.g., HIV), viral disorders, cancers, and inflammatory disorders (e.g., bacterial or viral infection, psoriasis, septicemia, arthritis, allergic reactions). INTERCEPT 307 nucleic acids, proteins and modulators thereof can be used to treat atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy.

INTERCEPT 307 exhibits homology to PB39. Therefore, INTERCEPT 307 nucleic acids, proteins and modulators thereof can be utilized to modulate the proliferation, differentiation, and/or function of prostate cells. INTERCEPT 307 nucleic acids, proteins and modulators thereof can be utilized to modulate processes involved in prostate development, differentiation and activity, including, but not limited to development, and differentiation and activation of prostate tissues and cells as well as any function associated with such cells, and amelioration of one or more symptoms associated with abnormal function of such cell types or disorders associated with such cell types. Such disorders can include, but are not limited to, malignant or benign prostate cell growth or inflammatory disorders (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and/or atypical prostatic stromal lesions).

In further light of the fact that INTERCEPT 307 exhibits homology to PB39 which is expressed by tumor cells, INTERCEPT 307 nucleic acids, proteins and modulators thereof can be utilized to modulate the development and progression of cancerous and non-cancerous cell proliferative disorders, such as deregulated proliferation (such as hyperdysplasia, hyper-IgM syndrome, or lymphoproliferative disorders), cirrhosis of the liver (a condition in which scarring has overtaken normal liver regeneration processes), treatment of keloid (hypertrophic scar) formation (disfiguring of the skin in which the scaring process interferes with normal renewal), psoriasis (a common skin condition characterized by excessive proliferation of the skin and delay in proper cell fate determination), benign tumors, fibrocystic conditions, and tissue hypertrophy (e.g., prostatic hyperplasia), or cancers, such as neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenström's macroglobulinemia.

In particular, INTERCEPT 307 nucleic acids, proteins and modulators thereof can be utilized to modulate the development and progression of prostate cancer, e.g., prostatic intraepithelial neoplasia, prostatic paraganglioma, and prostate adenocarcinoma.

As INTERCEPT 307 has a gas vesicle protein-like domain, INTERCEPT 307 nucleic acids and protein fragments that contain the gas vesicle protein-like domain can be used to produce gas vesicles which can be used for protein, drug, and antigen presentation (e.g., vaccines).

INTERCEPT 307 has a leucine zipper pattern, therefore, INTERCEPT 307 nucleic acids, proteins and modulators thereof can be used to modulate protein-protein interactions (e.g., stabilize, promote, inhibit or disrupt protein-protein interactions).

As INTERCEPT 307 has homology to eosinophil granule basic protein, INTERCEPT 307 nucleic acids, proteins and modulators thereof can be used to modulate eosinophil function and activity, e.g., the ability to kill targets such as helminth, protozoa, and bacteria. INTERCEPT 307 nucleic acids, proteins and modulators thereof can be used to treat asthma, allergies (e.g., ocular allergies), nonallergic ophthalmic diseases (e.g., Wegener's granulomatosis, orbital pseudo-tumor and histiocytosis X), and helminth infection. INTERCEPT 307 nucleic acids, proteins and modulators thereof can be used to monitor an individual's asthma condition.

INTERCEPT 307 expression can be utilized as a marker (e.g., an in situ marker) for specific tissues (e.g., the prostate) and/or cells (e.g., prostatic cells) in which INTERCEPT 307 is expressed. INTERCEPT 307 expression can also be utilized as a marker for the development and/or progression of diseases and disorders such as prostate cancer and asthma. Further, INTERCEPT 307 nucleic acids be utilized for chromosomal mapping, or as chromosomal markers, e.g., in radiation hybrid mapping.

### Human MANGO 511

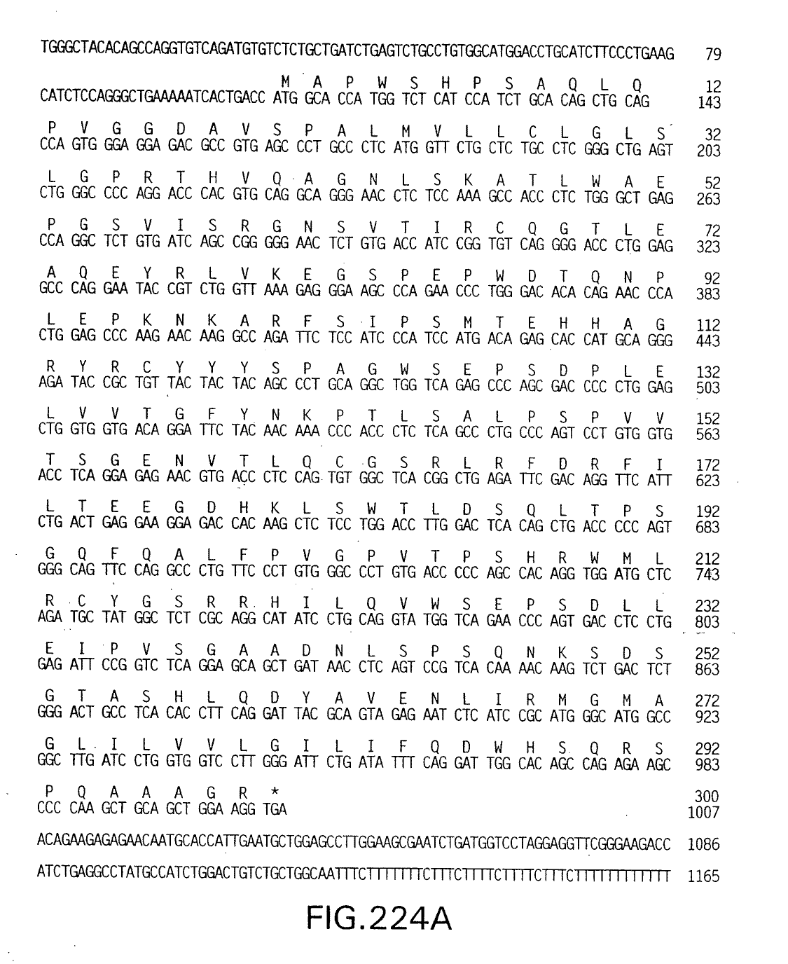

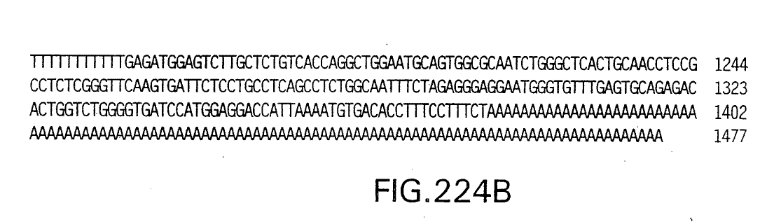

A cDNA encoding human MANGO 511 was identified by analyzing the sequences of clones present in a human dendritic cell library for sequences that encode wholly secreted or transmembrane proteins. This analysis led to the identification of a clone, jThxh005c10, encoding full-length human MANGO 511. The human MANGO 511 cDNA of this clone is 1477 nucleotides lbng (FIG. 224A-224B; SEQ ID NO:143). The open reading frame of this cDNA (nucleotides 108 to 1004 of SEQ ID NO:143) encodes a 299 amino acid transmembrane protein (SEQ ID NO: 144).

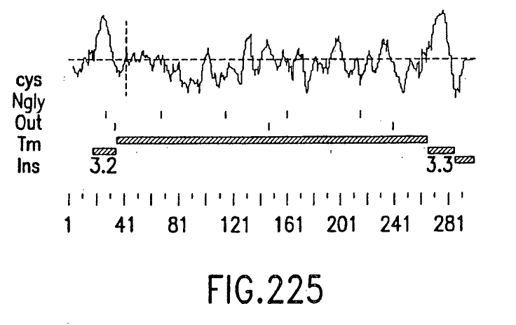

FIG. 225 depicts a hydropathy plot of human MANGO 511.

The signal peptide prediction program SIGNALP (Nielsen et al., 1997, Protein Engineering 10:1-6) predicted that human MANGO 511 includes a 41 amino acid signal peptide (amino acid 1 to amino acid 41), preceding the mature MANGO 511 protein corresponding to amino acid 42 to amino acid 299. In instances wherein the signal peptide is cleaved, the molecular weight of MANGO 511 protein without post-translational modifications is 32.8 kDa prior to the cleavage of the signal peptide, and 28.6 kDa after cleavage of the signal peptide.

Human MANGO 511 protein is a transmembrane protein that contains an extracellular domain at amino acid residues 42 to 265, a transmembrane domain at amino acid residues 266 to 284, and a cytoplasmic domain at amino acid residues 285 to 299 of SEQ ID NO:144.

In instances wherein the signal peptide is not cleaved, a human MANGO 511 protein is a transmembrane protein that contains an extracellular domain at amino acid residues 1 to 265, a transmembrane domain at amino acid residues 266 to 284, and a cytoplasmic domain at amino acid residues 285 to 299 of SEQ ID NO: 144.

Alternatively, in another embodiment, a human MANGO 511 protein is a transmembrane protein that contains a cytoplasmic domain at amino acid residues 42 to 265, a transmembrane domain at amino acid residues 266 to 284, and an extracellular domain at amino acid residues 285 to 299 of SEQ ID NO:144.

In one embodiment a cDNA sequence of human MANGO 511 has a nucleotide at position 138 which is cytosine (C). In this embodiment, the cDNA contains an open reading frame encoding a polypeptide having an amino acid at position 11 that is leucine (L). In an alternative embodiment, a species variant cDNA sequence of human MANGO 511 has a nucleotide at position 138 which is adenine (A). In this embodiment, the cDNA contains an open reading frame encoding a polypeptide having an amino acid at position 11 that is isoleucine (I), i.e., a conservative substitution.

In another embodiment a cDNA sequence of human MANGO 511 has a nucleotide at position 156 which is guanine (G). In this embodiment, the cDNA contains an open reading frame encoding a polypeptide having an amino acid at position 17 that is aspartate (D). In an alternative embodiment, a species variant cDNA sequence of human MANGO 511 has a nucleotide at position 156 which is adenine (A). In this embodiment, the cDNA contains an open reading frame encoding a polypeptide having an amino acid at position 17 that is asparagine (N), i.e., a conservative substitution.

In another embodiment a cDNA sequence of human MANGO 511 has a nucleotide at position 202 which is guanine (G). In this embodiment, the cDNA contains an open reading frame encoding a polypeptide having an amino acid at position 32 that is serine (S). In an alternative embodiment, a species variant cDNA sequence of human MANGO 511 has a nucleotide at position 202 which is cytosine (C). In this embodiment, the cDNA contains an open reading frame encoding a polypeptide having an amino acid at position 32 that is threonine (T), i.e., a conservative substitution.

In another embodiment a cDNA sequence of human MANGO 511 has a nucleotide at position 214 which is guanine (G). In this embodiment, the cDNA-contains an open reading frame encoding a polypeptide having an amino acid at position 36 that is arginine (R). In an alternative embodiment, a species variant cDNA sequence of human MANGO 511 has a nucleotide at position 214 which is adenine (A). In this embodiment, the cDNA contains an open reading frame encoding a polypeptide having an amino acid at position 36 that is lysine (K), i.e., a conservative substitution.

Human MANGO 511 includes an Ig-like domain at amino acids 60 to 118 of SEQ ID NO:144.

Human MANGO 511 has three N-glycosylation sites. The first has the sequence NLSK (at amino acid residues 43 to 46), the second has the sequence NVTL (at amino acid residues 157 to 160), and the third has the sequence NKSD (at amino acid residues 248 to 251). Four protein kinase C phosphorylation sites are present in MANGO 511. The first has the sequence TIR (at amino acid residues 64 to 66), the second has the sequence SHR (at amino acid residues 207 to 209), the third has the sequence SRR (at amino acid residues 217 to 219), and the fourth has the sequence SQR (at amino acid residues 289 to 291). MANGO 511 has three casein II kinase phosphorylation sites. The first has the sequence SMTE (at amino acid residues 105 to 108) and the second has the sequence TSGE (at amino acid residues 153 to 156). Five N-myristylation sites are present in MANGO 511. The first has the sequence GSVISR (at amino acid residues 54 to 59), the second has the sequence GNSVTI (at amino acid residues 60 to 65), the third has the sequence GTLEAQ (at amino acid residues 69 to 74), the fourth has the sequence GQFQAL (at amino acid residues 193 to 198), and the fifth has the sequence GAADNL (at amino acid residues 238 to 243)

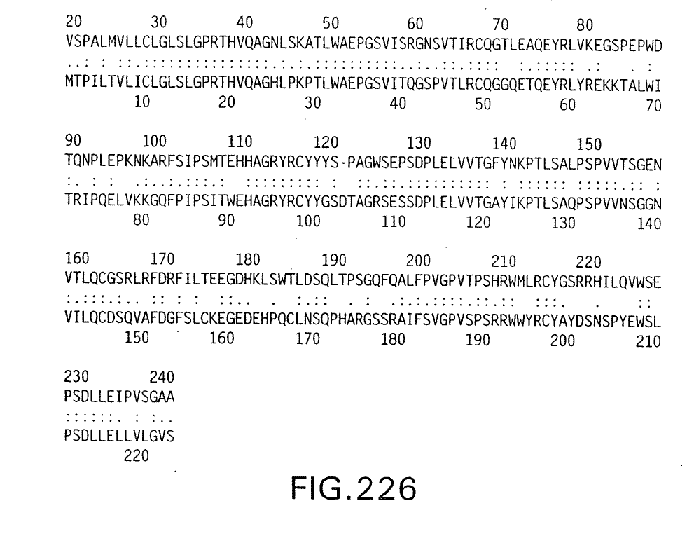

FIG. 226 shows a local alignment of the human MANGO 511 amino acid sequence with the leukocyte Ig-like receptor-1 (LIR-1) amino acid sequence (Accession Number AAB63522). The alignment shows that there is a 59.2% local identity over the 233 amino acids that were compared from the sequences of MANGO 511 and LIR-1.

LIR-1 is a expressed by lymphocytes, natural killer cells, monocytes, and dendritic cells and has been shown to be a major histocompatibility complex (MHC) class I binding protein (Cosman et al. (1997) Immunity 7:273-282). LIR-1 can function as a broad HLA class I-specific inhibitory receptor that recognizes different alleles coded for by different HLA loci (Vitale et al. (1999) Int. Immunol. 11:29-35). The tyrosine phosphorylation of LIR-1 in monocytes has been shown to result in the binding of tyrosine phosphatase SHP-1, and LIR-1 has been shown to be involved in the inhibition or down-modulation of monocyte activation signals (Fanger et al; (1998) Eur. J. Immunol. 28:3423-3434). As such MANGO 511 nucleic acids, proteins and modulators thereof are useful in modulating MHC class I binding and monocyte activation.

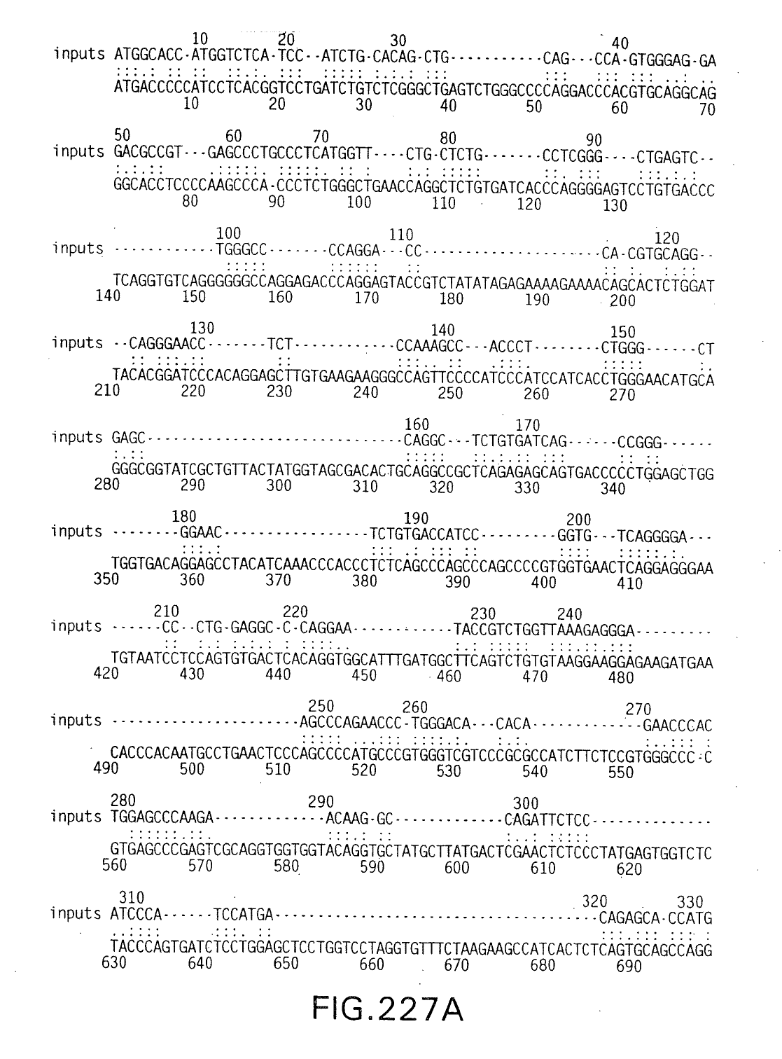

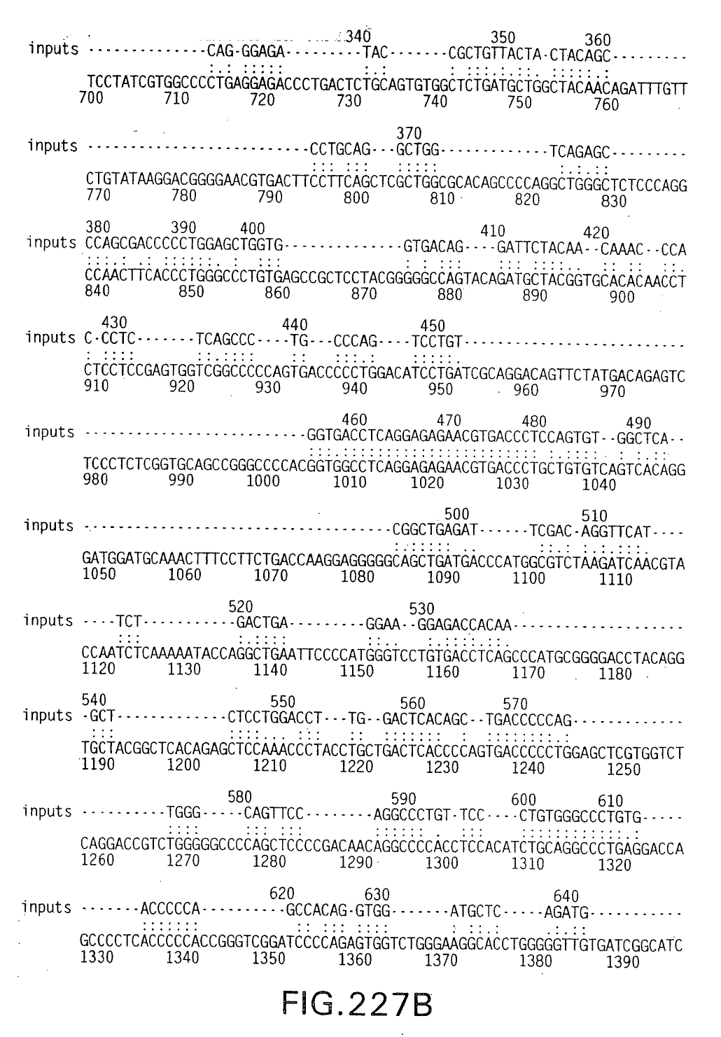

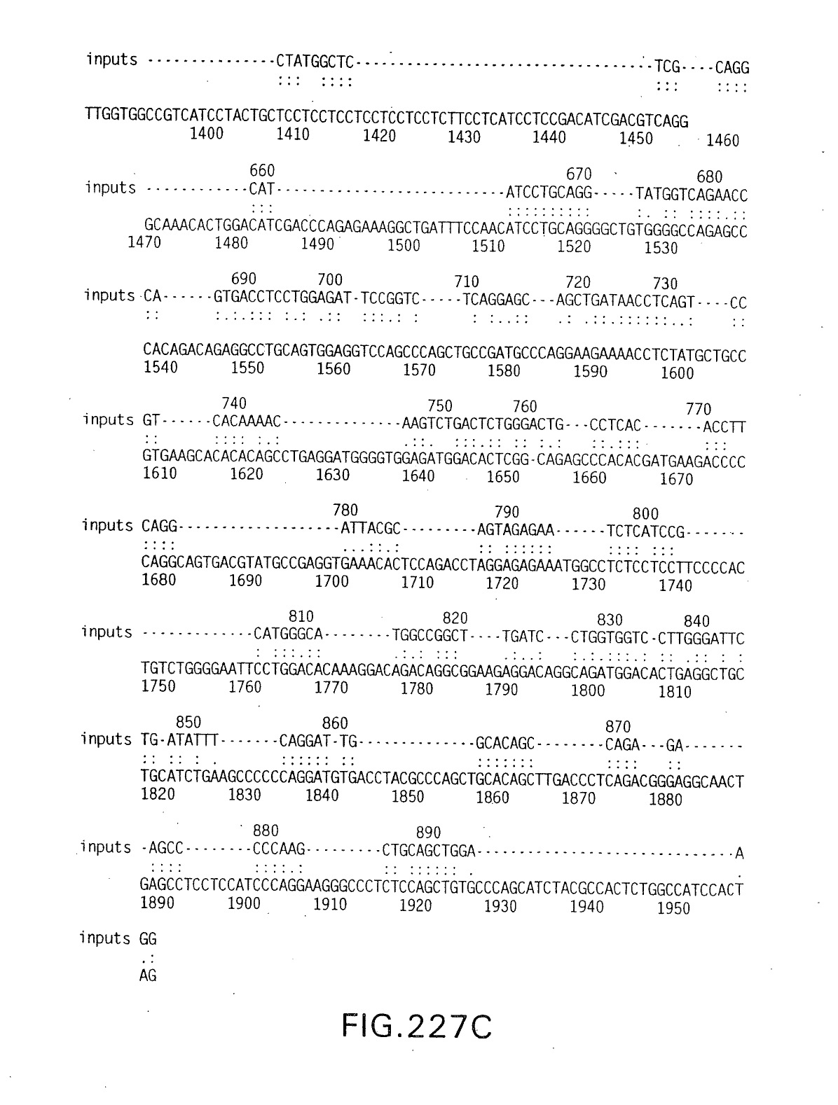

FIG. 227A-227C shows an alignment of the coding regions of the nucleotide sequence of LIR-1 (Accession Number AF009221) and the nucleotide sequence of human MANGO 511. The alignment shows a 34.0% overall sequence identity between the two nucleotide sequences. The coding region of the nucleotide sequence of LIR-1 (Accession Number AF009221) and the full-length nucleotide sequence of human MANGO 511 cDNA have an overall sequence identity of 44.0%.

Clone EpM511, which encodes human MANGO 511, was deposited with the American Type Culture Collection (10801 University Boulevard, Manassas, Va. 20110-2209) on Jul. 23, 1999 and assigned Accession Number PTA-425. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of MANGO 511 Nucleic Acids, Polypeptides, and Modulators Thereof

As MANGO 511 was originally found in a dendritic cell library, and including one or more Ig-like domains, MANGO 511 nucleic acids, proteins, and modulators thereof can be used to modulate the proliferation, development, differentiation, and/or function of immune cells, e.g. B-cells, dendritic cells, natural killer cells and monocytes, and/or immune function. MANGO 511 nucleic acids, proteins and modulators thereof can be utilized to modulate immunoglobulins and formation of antibodies, and immune-related processes, e.g., the host immune response by, for example, modulating the formation of and/or binding to immune complexes, detection and defense against surface antigens and bacteria, and immune surveillance for rapid removal or pathogens. Such MANGO 511 compositions and modulators thereof can be utilized modulate or treat immune disorders that include, but are not limited to, immune proliferative disorders (e.g., carcinoma, lymphoma, e.g., follicular lymphoma), and disorders associated with fighting pathogenic infections, e.g., bacterial (e.g., chlamydia) infection, parasitic infection, and viral infection (e.g., HSV or HIV infection), and pathogenic disorders associated with immune disorders (e.g., immunodeficiency disorders, such as HIV), autoimmune disorders, such as arthritis, graft rejection (e.g., allograft rejection), multiple sclerosis, Grave's disease, or Hashimoto's disease, T cell disorders (e.g., AIDS) and inflammatory disorders, such as septicemia, cerebral malaria, inflammatory bowel disease, arthritis (e.g., rheumatoid arthritis, osteoarthritis), and allergic inflammatory disorders (e.g., asthma, psoriasis), apoptotic disorders (e.g., rheumatoid arthritis, systemic lupus erythematosus, insulin-dependent diabetes mellitus), cytotoxic disorders, septic shock, and cachexia.

MANGO 511 nucleic acids, proteins, and modulators thereof can also be used to modulate leukocyte trafficking, cancer, Type I immunologic disorders, e.g., anaphylaxis and/or rhinitis, by, for example, modulating the interaction between antigens and cell receptors, e.g., high affinity IgE receptors.

As MANGO 511 exhibits homology to leukocyte Ig-like receptor-1 (LIR-1), MANGO 511 nucleic acids, proteins and modulators thereof can be used modulate MHC class I binding. For example, MANGO 511 nucleic acids, proteins and modulators thereof can be used to modulate or treat disorders associated with aberrant MHC class I binding, such as autoimmune disorders, bacterial infections and viral infections. MANGO 511 nucleic acids, proteins and modulators thereof can be used to modulate monocyte activation signals and may be used to inhibit unwanted bystander responses mediated by antigen-specific T-cells. For example, antagonists of MANGO 511 action, such as peptides, antibodies or small molecules, that decrease or prevent MANGO 511 signaling can be used as modulators of monocyte activation. MANGO 511 nucleic acids, proteins and modulators thereof can be used to modulate or treat disorders associated with aberrant monocyte activation including, but not limited to, Wegener's granulomatosis (WG), hemophagocytic lymphohistiocytosis (HLH), histiocytic medullary reticulosis (HM), sarcoidosis, polyneuropathy, organomegaly, endocrinopathy, M protein, skin changes (POMEMS) syndrome, and systemic sclerosis (Ssc).

As MANGO 511 exhibits homology to LIR-1, MANGO 511 nucleic acids, proteins and/or modulators thereof can be used to modulate natural killer cell function, e.g., activation. MANGO 511 nucleic acids, proteins and modulators thereof can be used to treat diseases associated with aberrant natural killer cell activation such as chronic natural killer cell lymphocytosis, aggressive non-T, non-B natural killer cell lymphoma/leukemia (ANKL/L), and Chediak-Higashi syndrome.

MANGO 511 expression can be utilized as a marker (e.g., an in situ marker) for specific tissues (e.g., the spleen) and/or cells (e.g., immune cells such as dendritic cells and natural killer cells) in which MANGO 511 is expressed. MANGO 511 nucleic acids can also be utilized for chromosomal mapping, or as chromosomal markers, e.g., in radiation hybrid mapping.

### Human TANGO 361

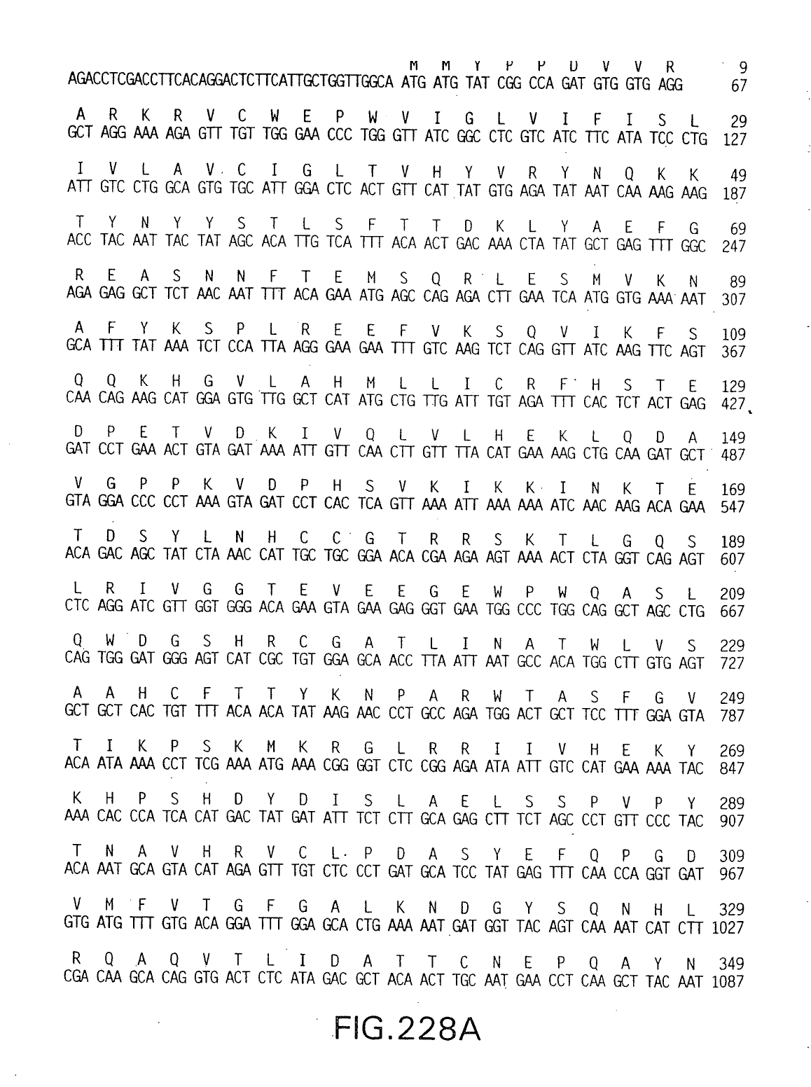

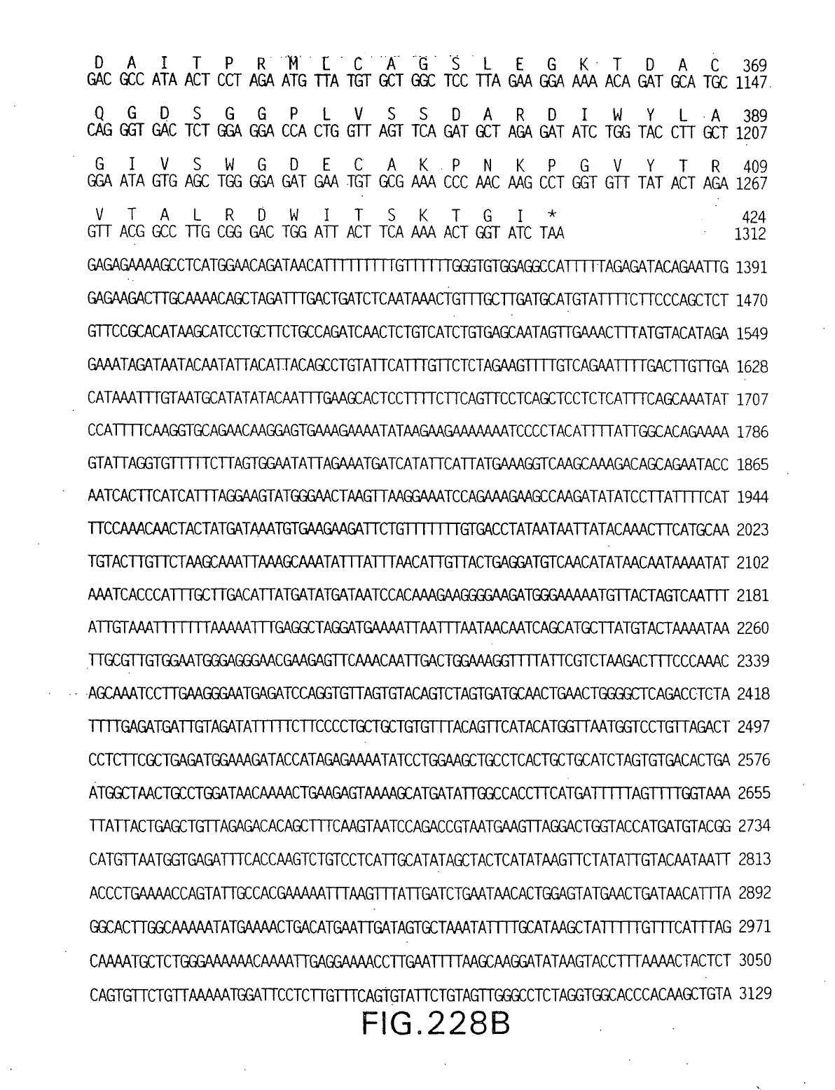

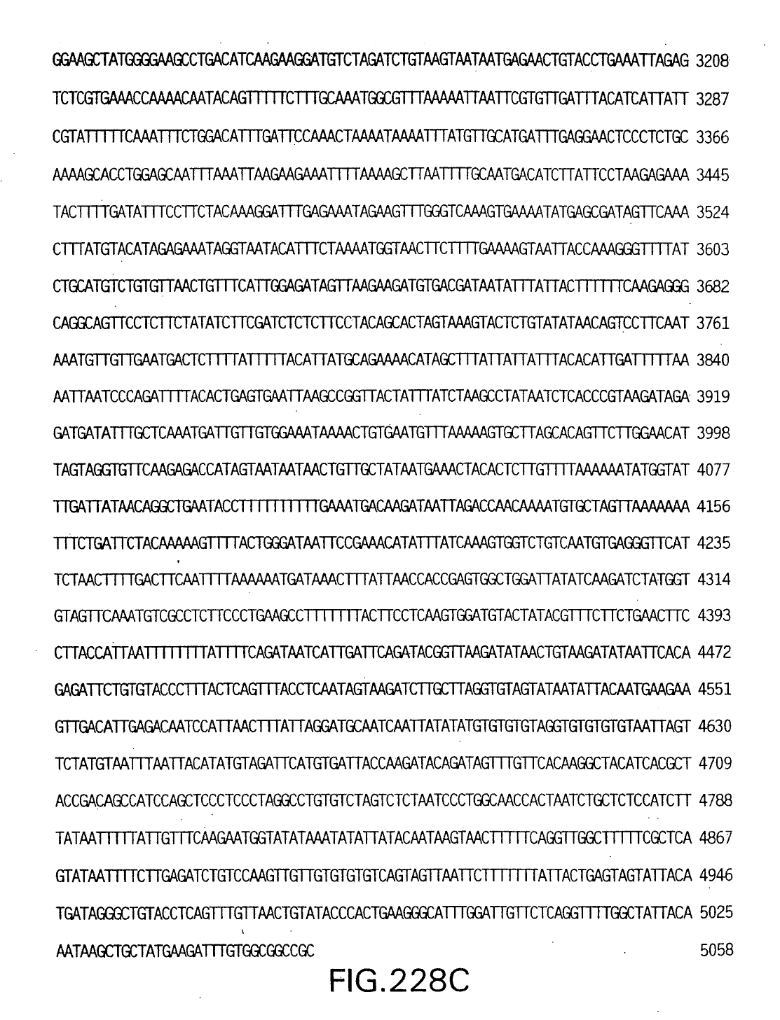

A cDNA encoding human TANGO 361 was identified by analyzing the sequences of clones present in a prostate epithelium library for sequences that encode wholly secreted or transmembrane proteins. This analysis led to the identification of a clone, jthqb014c05, encoding full-length human TANGO 361. The human TANGO 361 cDNA of this clone is 5058 nucleotides long (FIG. 228A-228C; SEQ ID NO:145). The open reading frame of this cDNA (nucleotides 41 to 1309 of SEQ ID NO: 145) encodes a 423 amino acid transmembrane protein (SEQ ID NO:146).

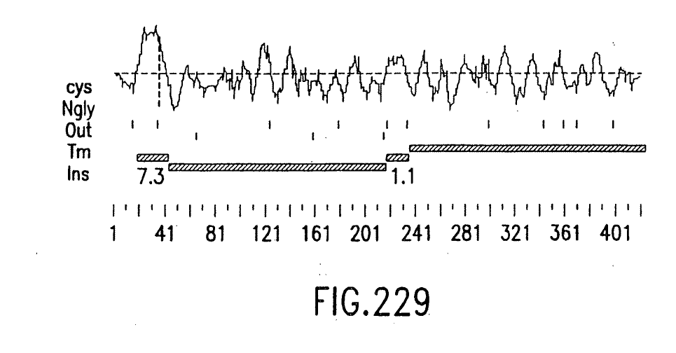

FIG. 229 depicts a hydropathy plot of human TANGO 361.

The signal peptide prediction program SIGNALP (Nielsen et al., 1997, Protein Engineering 10:1-6) predicted that human TANGO 361 includes a 35 amino acid signal peptide (amino acid 1 to amino acid 35 of SEQ ID NO:146) preceding the mature TANGO 361 protein (corresponding to amino acid 36 to amino acid 423 of SEQ ID NO:146). In instances wherein the signal peptide is cleaved, the molecular weight of TANGO 361 protein without post-translational modifications is 47.7 kDa prior to the cleavage of the signal peptide, and 43.6 kDa after cleavage of the signal peptide.

Human TANGO 361 protein is a transmembrane protein that contains an extracellular domain at amino acid residues 235 to 423, a transmembrane domain at amino acid residues 217 to 234, and a cytoplasmic domain at amino acid residues 36 to 216 of SEQ ID NO:146.

In instances wherein the signal peptide is not cleaved, human TANGO 361 contains an extracellular domain at amino acid residues 235 to 423, a transmembrane domain at amino acid residues 217 to 234, and a cytoplasmic domain at amino acid residues 1 to 216 of SEQ ID NO: 146.

Alternatively, in another embodiment, a human TANGO 361 protein contains a cytoplasmic domain at amino acid residues 235 to 423, a transmembrane domain at amino acid residues 217 to 234, and an extracellular domain at amino acid residues 36 to 216 of SEQ ID NO: 146.

In one embodiment a cDNA sequence of human TANGO 361 has a nucleotide at position 63 which is thymine (T). In this embodiment, the cDNA contains an open reading frame encoding a polypeptide having an amino acid at position 8 that is valine (V). In an alternative embodiment, a species variant cDNA sequence of human TANGO 361 has a nucleotide at position 63 which is cytosine (C). In this embodiment, the cDNA contains an open reading frame encoding a polypeptide having an amino acid at position 8 that is alanine (A), i.e., a conservative substitution.

In another embodiment a cDNA sequence of human TANGO 361 has a nucleotide at position 66 which is guanine (G). In this embodiment, the cDNA contains an open reading frame encoding a polypeptide having an amino acid at position 9 that is arginine (R). In an alternative embodiment, a species variant cDNA sequence of human TANGO 361 has a nucleotide at position 66 which is adenine (A). In this embodiment, the cDNA contains an open reading frame encoding a polypeptide having an amino acid at position 9 that is lysine (K), i.e., a conservative substitution.

In another embodiment a cDNA sequence of human TANGO 361 has a nucleotide at position 117 is thymine (T). In this embodiment, the cDNA contains an open reading frame encoding a polypeptide having an amino acid at position 15 that is phenylalanine (F). In an alternative embodiment, a species variant cDNA sequence of human TANGO 361 has a nucleotide at position 117 which is adenine (a). In this embodiment, the cDNA contains an open reading frame encoding a polypeptide having an amino acid at position 26 that is tyrosine (Y), i.e., a conservative substitution.

In another embodiment a cDNA sequence of human TANGO 361 has a nucleotide at position 122 is thymidine (T). In this embodiment, the cDNA contains an open reading frame encoding a polypeptide having an amino acid at position 28 that is serine (S). In an alternative embodiment, a species variant cDNA sequence of human TANGO 361 has a nucleotide at position 122 which is adenine (A). In this embodiment, the cDNA contains an open reading frame encoding a polypeptide having an amino acid at position 28 that is threonine (T), i.e., a conservative substitution.

Human TANGO 361 includes a serine protease domain at amino acids 192 to 417 of SEQ ID NO:146.

TANGO 361 has three N-glycosylation sites with the first sequence NFTE at amino acid residues 75 to 78, the second sequence NKTE at amino acid residues 166 to 169, NATW at amino acid residues 223 to 226.

Ten protein kinase C phosphorylation sites are present in TANGO 361. The first has the sequence TDK (at amino acid residues 61 to 63, the second has the sequence SQR (at amino acid residues 80 to 82, the third has the sequence SVK (at amino acid residues 159 to 161, the fourth has the sequence TRR (at amino acid residues 180 to 182, the fifth has the sequence SLR (at amino acid residues 189 to 191, the sixth has the sequence SHR (at amino acid residues 214 to 216, the seventh has the sequence TYK (at amino acid residues 236 to 238, the eighth has the sequence TIK (at amino acid residues 250 to 252, the ninth has the sequence TPR (at amino acid residues 353 to 355, and the tenth has the sequence TSK (at amino acid residues 418 to 420.

TANGO 361 has seven casein kinase II phosphorylation sites. The first has the sequence STED (at amino acid residues 127 to 130, the second has the sequence TETD (at amino acid residues 168 to 171, the third has the sequence TEVE (at amino acid residues 196 to 199, the fourth has the sequence SLAE (at amino acid residues 279 to 282, the fifth has the sequence TLID (at amino acid residues 335 to 338, the sixth has the sequence TCNE (at amino acid residues 341 to 344, and the seventh has the sequence SWGD (at amino acid residues 393 to 396.

Four N-myristylation sites are present in TANGO 361. The first has the sequence GTRRSK (at amino acid residues 179 to 184, the second has the sequence GSHRCG (at amino acid residues 213 to 218), the third has the sequence GALKND (at amino acid residues 317 to 322), and the fourth has the sequence GSLEGK (at amino acid residues 360 to 365).

TANGO 361 has a ATP/GTP binding site motif with the sequence AGSLEGKT (at amino acid residues 359 to 366). TANGO 361 has a serine protease, histidine active site, consensus sequence with the sequence VSAAHC (at amino acid residues 228 to 233).

TANGO 361 has a serine protease, serine active site, consensus sequence with the sequence GDSGG (at amino acid residues 371 to 375).

Clone EpT361, which encodes TANGO 361, was deposited with the American Type Culture Collection (10801 University Boulevard, Manassas, Va. 20110-2209) on Jul. 29, 1999 and assigned Accession Number PTA-438. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 361 Nucleic Acids, Polypeptides, and Modulators Thereof

As TANGO 361 was originally found in a prostate epithelium library, TANGO 361 nucleic acids, proteins, and modulators thereof can be used to modulate the proliferation, differentiation, and/or function of prostate cells and tissues, and to ameliorate of one or more symptoms associated with abnormal function of such cells or tissues or disorders associated with such cells or tissues. Such disorders can include, but are not limited to, malignant or benign prostate cell growth or inflammatory disorders (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, atypical prostatic stromal lesions).

TANGO 361 has structural homology with serine proteases. As such TANGO 361 nucleic acids, proteins and modulators thereof can be utilized to modulate activities, processes or disorders associated with protease activity, e.g., serine protease activity. For example, TANGO 361 nucleic acids, proteins or modulators thereof can be used to modulate serine protease activities, such as those activities associated with such serine proteases (or, where appropriate, human homologs thereof), e.g., adipsin (complement factor D), acrosin, thrombin, plasminogen, protein C, cathepsin G, chymotrypsin, complement components and signaling, cytotoxic cell proteases, duodenase I, elastases 1, 2, 3A, 3B and medullasin, enterokinase, bepatocyte growth factor activator, hepsin, kallikreins, gamrnma-renin, prostate specific antigen, mast cell proteases, myeloblastin, Alzheimer's plaque-related proteases, tryptases, ancrod, batroxobin, cerastobin, flavoxobin, apolipoprotein, blood fluke cercarial protease, Drosophila trypsin like protease (e.g., alpha, easter, and snake locus), Drosophila protease stubble, or major mite fecal antigen.

TANGO 361 nucleic acids, proteins and modulators thereof can be used to modulate processes and/or diseases involved with serine protease response activity. For example, such processes and/or diseases can include, but are not limited to cellular activation, cellular proliferation, motility and differentiation, the alternative complement pathway, e.g., disturbances of the complement regulation system, such as complement regulator deficiencies, which include, for example, hereditary angioedema (an allergic disorder) and proxysmal nocturnal hemoglobinuria (the presence of hemoglobin in the urine), modulate body weight or body weight disorders, e.g., obesity or cachexia, systemic energy balance and diabetes.

TANGO 361 nucleic acids, proteins and modulators thereof can also be used to modulate immune related diseases and disorders, such disorders can include, e.g., autoimmune disorders (e.g., arthritis, graft rejection (e.g., allograft rejection), and T cell autoimmune disorders (e.g., AIDS) and inflammatory disorders (e.g., bacterial infection, psoriasis, septicemia, cerebral malaria, inflammatory bowel disease, multiple sclerosis, arthritis (e.g., rheumatoid arthritis, osteoarthritis), and allergic inflammatory disorders (e.g., asthma, psoriasis).

TANGO 361 nucleic acids, proteins, and modulators thereof can also be used to rid the body of invading or infecting agents, e.g., bacteria, viruses, parasites, neoplastic cells, cellular platelet function, e.g., activation. Antagonists of TANGO 361 nucleic acids, proteins and modulators thereof, such as peptides, antibodies or small molecules that decrease or block TANGO 361 action, can be used as platelet antagonists, e.g. activation and aggregation blockers. In another example, agonists that mimic TANGO 361 activity, such as peptides, antibodies or small molecules, can be used to induce platelet function, e.g., activation and aggregation. TANGO 361 nucleic acids, proteins and modulators thereof can be utilized to modulate platelet-related processes and disorders, e.g., Glanzmann's thromboasthemia, which is a bleeding disorder characterized by failure of platelet aggregation in response to cell stimuli, and hereditary hemophilia. TANGO 361 nucleic acids, proteins and modulators thereof can be used to modulate formation of Alzheimer's plaques, treatment of Alzheimer's disease, treatment of Fanconi's anemia (FA), and symptoms associated with FA (e.g., bone marrow failure, a plastic anemia, infection, fatigue and/or spontaneous hemorrhage or bleeding).

TANGO 361 expression can be utilized as a marker (e.g. an in situ marker) for specific tissues (e.g., the prostate) and/or cells (e.g., prostatic cells) in which TANGO 361 is expressed. TANGO 361 nucleic acids can also be utilized for chromosomal mapping, or as chromosomal markers, e.g., in radiation hybrid mapping.

### Human TANGO 499 Form 1, Variant 1

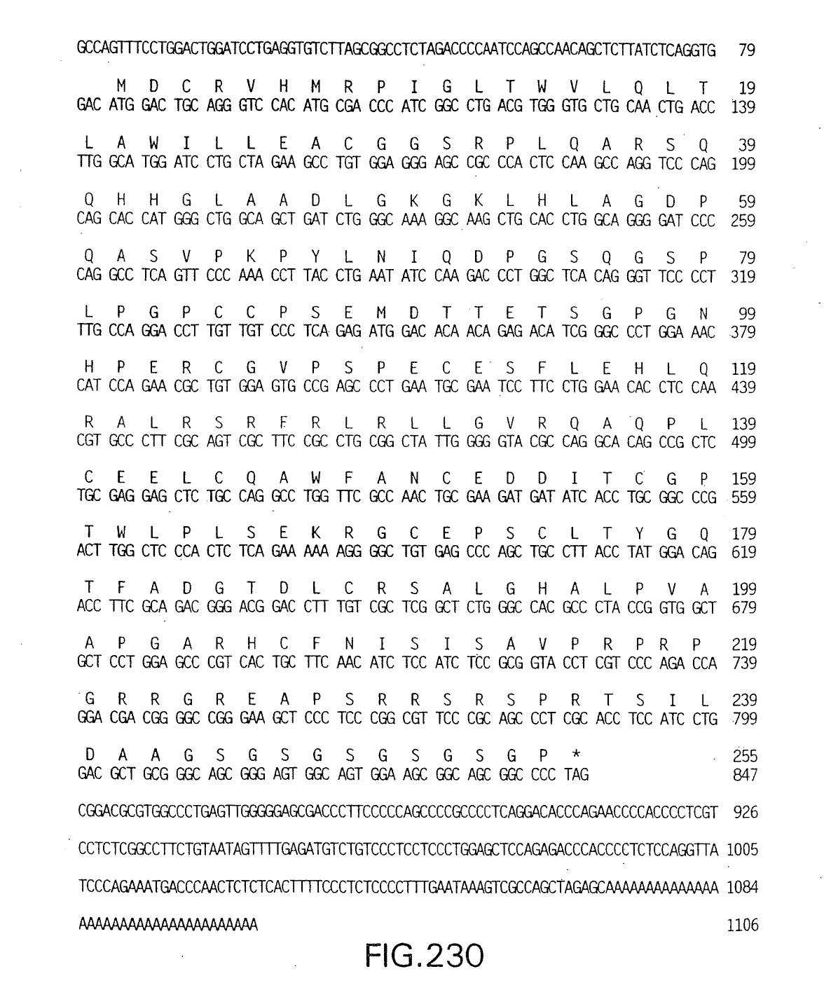

A cDNA encoding human TANGO 499 was identified by analyzing the sequences of clones present in a human pituitary library for sequences that encode wholly secreted or transmembrane proteins. This analysis led to the identification of a clone, jthbb 123c 10, encoding human TANGO 499. This form of human TANGO 499 is referred to herein as human TANGO 499 form 1, variant 1. The human TANGO 499 form 1, variant 1 cDNA of this clone is 1106 nucleotides long (FIG. 230; SEQ ID NO: 147).

In one embodiment, the open reading frame of a TANGO 499 cDNA is nucleotides 83 to 844, and encodes a human TANGO 499, form 1, variant 1 polypeptide comprising a 254 amino acid polypeptide (FIG. 230).

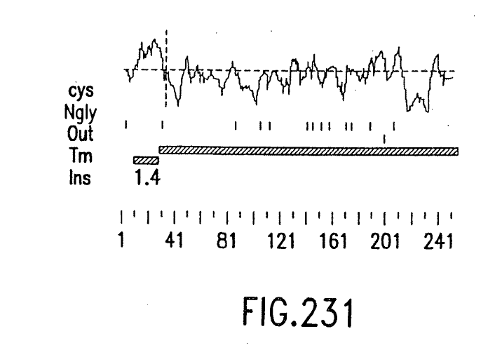

FIG. 231 depicts a hydropathy plot of human TANGO 499 form 1, variant 1.

The signal peptide prediction program SIGNALP (Nielsen et al., 1997, Protein Engineering 10:1-6) predicted that human TANGO 499 form 1, variant 1 includes a 30 amino acid signal peptide (amino acid 1 to amino acid 30 of SEQ ID NO: 148) preceding the mature TANGO 499 form 1, variant 1 protein (corresponding to amino acid 31 to amino acid 254 of SEQ ID NO: 148). In instances wherein the signal peptide is cleaved, the molecular weight of TANGO 499 form 1, variant 1 protein without post-translational modifications is 27.2 kDa prior to the cleavage of the signal peptide, and 23.8 kDa after cleavage of the signal peptide, thus TANGO 499 form 1, variant 1 polypeptides can be secreted and contain a sequence of amino acids 31 to 254 of SEQ ID NO: 148. In instances wherein the signal peptide is not cleaved, human TANGO 499 form 1, variant 1 is a secreted protein having amino acids 1 to 254 of SEQ D NO:148.

In one embodiment a cDNA sequence of human TANGO 499 form 1, variant 1 has a nucleotide at position 134 which is cytosine (C). In this embodiment, the cDNA contains an open reading frame encoding a polypeptide having an amino acid at position 18 that is leucine (L). In an alternative embodiment, a species variant cDNA sequence of human TANGO 499 form 1, variant 1 has a nucleotide at position 134 which is guanine (G). In this embodiment, the cDNA contains an open reading frame encoding a polypeptide having an amino acid at position 18 that is valine (1), i.e., a conservative substitution.

In another embodiment a cDNA sequence of human TANGO 499 form 1, variant 1 has a nucleotide at position 137 which is adenine (A). In this embodiment, the cDNA contains an open reading frame encoding a polypeptide having an amino acid at position 19 that is threonine (T). In an alternative embodiment, a species variant cDNA sequence of human TANGO 499 form 1, variant 1 has a nucleotide at position 137 which is thymine (T). In this embodiment, the cDNA contains an open reading frame encoding a polypeptide having an amino acid at position 19 that is serine (S), i.e., a conservative substitution.

In another embodiment a cDNA sequence of human TANGO 499 form 1, variant 1 has a nucleotide at position 192 which is guanine (G). In this embodiment, the cDNA contains an open reading frame encoding a polypeptide having an amino acid at position 37 that is arginine (R). In an alternative embodiment, a species variant cDNA sequence of human TANGO 499 form 1, variant 1 has a nucleotide at position 192 which is adenine (A).

In this embodiment, the cDNA contains an open reading frame encoding a polypeptide having an amino acid at position 37 that is lysine (K), i.e., a conservative substitution.

In another embodiment a cDNA sequence of human TANGO 499 form 1, variant 1 has a nucleotide at position 197 which is cytosine (C). In this embodiment, the cDNA contains an open reading frame encoding a polypeptide having an amino acid at position 39 that is glutamine (Q). In an alternative embodiment, a species variant cDNA sequence of human TANGO 499 form 1, variant 1 has a nucleotide at position 197 which is guanine (G). In this embodiment, the cDNA contains an open reading frame encoding a polypeptide having an amino acid at position 39 that is glutamate (E), i.e., a conservative substitution.

TANGO 499 form 1, variant 1 has an N-glycosylation site with the sequence NISI (at amino acid residues 95 to 98). Three glycosaminoglycan attachments sites are present in TANGO 499 form 1, variant 1. The first has the sequence SGPG (at amino acid residues 95 to 98), the second has the sequence SGSG (at amino acid residues 244 to 247), and the third has the sequence SGSG (at amino acid residues 248 to 251).

Three protein kinase C phosphorylation sites are present in TANGO 499 form 1, variant 1. The first has the sequence SEK (at amino acid residues 165 to 167), and the second has the sequence SRR (at amino acid residues 228 to 230), and the third has the sequence SPR (at amino acid residues 233 to 235). TANGO 499 form 1, variant 1 has four casein kinase II phosphorylation sites. The first has the sequence SEMD (at amino acid residues 87 to 90), the second has the sequence SFLE (at amino acid residues 113 to 116), the third has the sequence TFAD (at amino acid residues 180 to 183), and the fourth has the sequence SILD (at amino acid residues 237 to 240).

Six N-myristylafion sites are present in TANGO 499 form 1, variant 1. The first has the sequence GVRQAQ (at amino acid residues 132 to 137), the second has the sequence GCEPSC (at amino acid residues 169 to 174), the third has the sequence GQTFAD (at amino acid residues 178 to 183), the fourth has the sequence GTDLCR (at amino acid residues 184 to 189), the fifth has the sequence GARHCF (at amino acid residues 202 to 207, and the sixth has the sequence GSGSGS (at amino acid residues 243 to 248).

TANGO 499 form 1, variant 1 has an amidation site with the sequence PGRR (at amino acid residues 219 to 222).

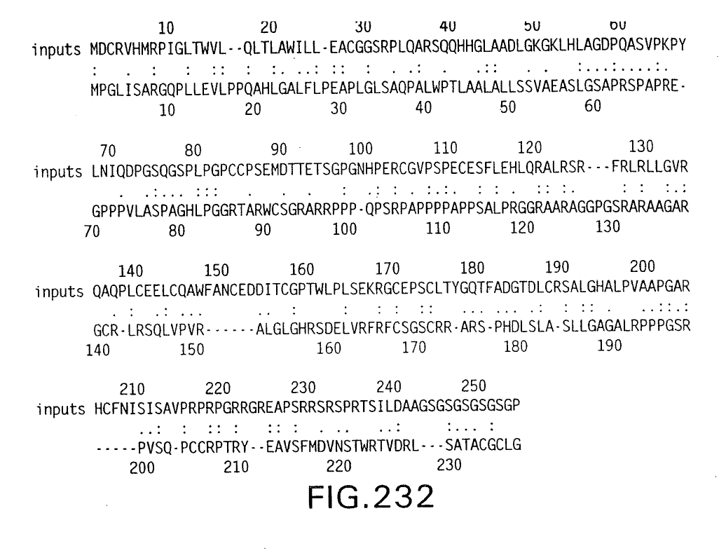

FIG. 232 shows an alignment of the human TANGO 499 form 1, variant 1 with the Artemin amino acid sequence. The alignment shows that there is a 23.5% overall amino acid sequence identity between TANGO 499 form 1, variant 1 and Artemin. The Artemin protein is widely expressed in the nervous system that has been shown to be involved in such processes as peripheral neuron survival and also dopaminergic midbrain neuron survival.

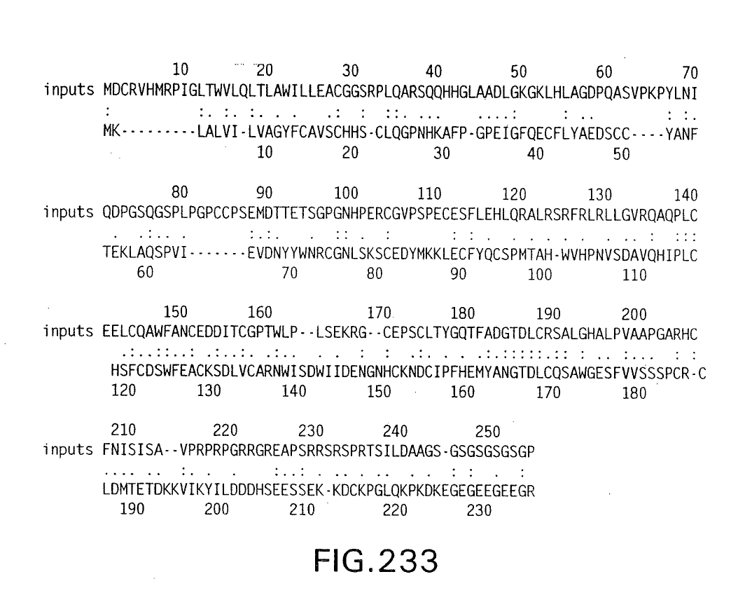

Human TANGO 499 form 1, variant 1 contains Glial cell line-derived neurotrophic factor (GDNF) and riboflavin binding protein homology. FIG. 233 shows an alignment of the nucleotide sequence of Riboflavin binding protein and the amino acid sequence of TANGO 499 form 1, variant 1. The alignment shows a 44.5% overall sequence identity between the two nucleotide sequences. The Riboflavin binding protein is expressed in germinal tissues and is involved in the development and maturation of the embryo.

Clone EpT499, which encodes TANGO 499 form 1, variant 1, was deposited with the American Type Culture Collection (10801 University Boulevard, Manassas, Va. 20110-2209) on Aug. 5, 1999 and assigned Accession Number PTA-455. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Human TANGO 499 Form 2, Variant 3

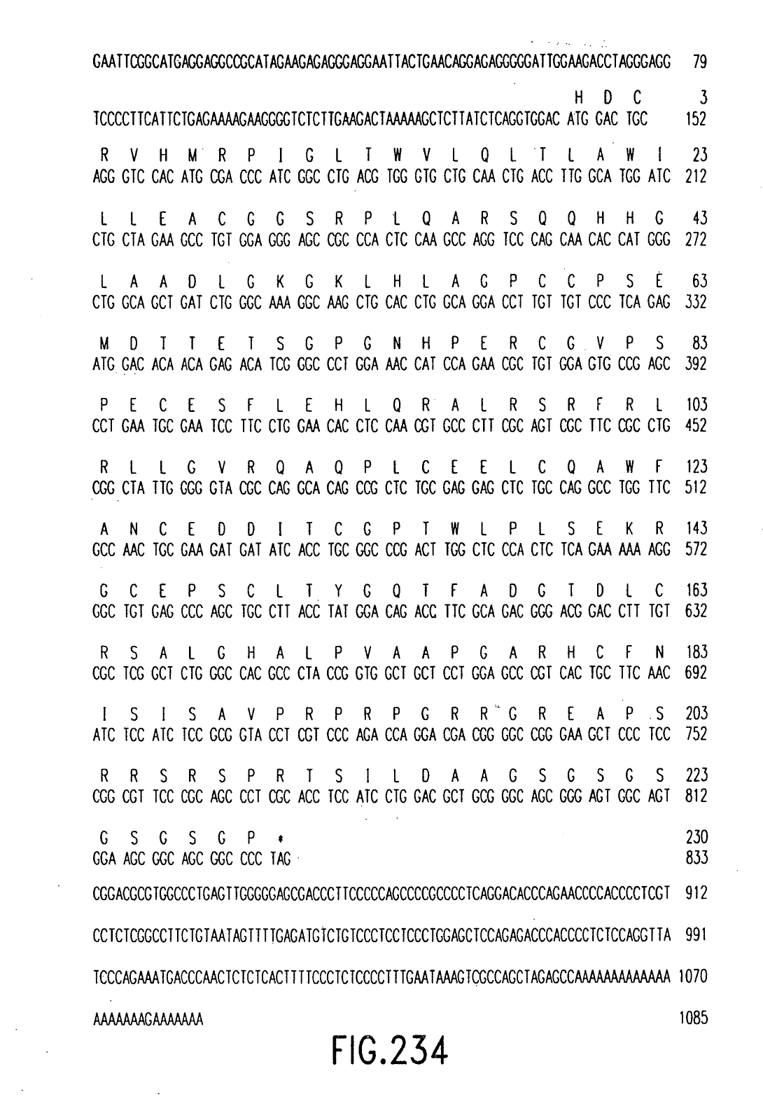

A cDNA encoding human an additional form of TANGO 499 was identified by analyzing the sequences of clones present in a retina library for sequences that encode wholly secreted or transmembrane proteins. This analysis led to the identification of a clone, AthX435e8, encoding human TANGO 499. This sequence is referred to herein as form 2, variant 3. The human TANGO 499 form 2, variant 3 cDNA of this clone is 1085 nucleotides long (FIG. 234; SEQ ID NO:149).

In one embodiment, the open reading frame of this cDNA is from nucleotides 144 to 8301 and encodes a 229 amino acid secreted protein (SEQ ID NO: 150).

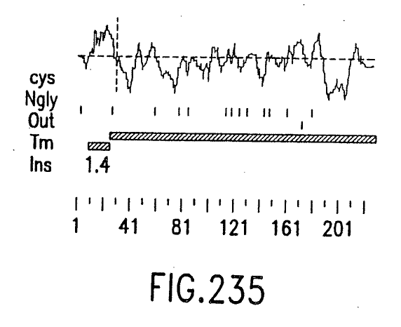

FIG. 235 depicts a hydropathy plot of human TANGO 499 form 2, variant 3.

The signal peptide prediction program SIGNALP (Nielsen et al., 1997, Protein Engineering 10:1-6) predicted that human TANGO 499 form 2, variant 3 includes a 30 amino acid signal peptide (amino acid 1 to amino acid 30 of SEQ ID NO: 150) preceding the mature TANGO 499 form 2, variant 3 protein (corresponding to amino acid 31 to amino acid 229 of SEQ ID NO:150). In instances wherein the signal peptide is cleaved, the molecular weight of TANGO 499 form 2, variant 3 protein without post-translational modifications is 24.6 kDa prior to the cleavage of the signal peptide, and 21.2 kDa after cleavage of the signal peptide, thus TANGO 499 form 2, variant 3 polypeptides can be secreted and contain a sequence of amino acids 31 to 229 of SEQ ID NO: 150.

TANGO 499 form 2, variant 3 has an N-glycosylation site with the sequence NISI (at amino acid residues 95 to 98).

Three glycosaminoglycan attachments sites are present in TANGO 499 form 2, variant 3. The first has the sequence SGPG (at amino acid residues 70 to 73), the second has the sequence SGSG (at amino acid residues 219 to 222), and the third has the sequence SGSG (at amino acid residues 223 to 246).

Three protein kinase C phosphorylation sites are present in TANGO 499 form 2, variant 3. The first has the sequence SEK (at amino acid residues 140 to 152), and the second has the sequence SRR (at amino acid residues 203 to 205), and the third has the sequence SPR (at amino acid residues 208 to 210). TANGO 499 form 2, variant 3 has four casein kinase II phosphorylation sites. The first has the sequence SEMD (at amino acid residues 62 to 65), the second has the sequence SFLE (at amino acid residues 88 to 91), the third has the sequence TFAD (at amino acid residues 155 to 158), and the fourth has the sequence SILD (at amino acid residues 212 to 215).

Six N-myristylation sites are present in TANGO 499 form 2, variant 3. The first has the sequence GVRQAQ (at amino acid residues 107 to 112), the second has the sequence GCEPSC (at amino acid residues 144 to 149, the third has the sequence GQTFAD (at amino acid residues 153 to 158), the fourth has the sequence GTDLCR (at amino acid residues 159 to 164), the fifth has the sequence GARHCF (at amino acid residues 177 to 182), and the sixth has the sequence GSGSGS (at amino acid residues 218 to 223).

TANGO 499 form 2, variant 3 has an amidation site with the sequence PGRR (at amino acid residues 194 to 197).

Human TANGO 499 form 2, variant 3 includes Glial cell line-derived neurotrophic factor (GDNF) and riboflavin binding protein homology. The Riboflavin binding protein is expressed in germinal tissues and is involved in the development and maturation of the embryo. Loss of expression of the riboflavin binding protein results in the failure of the embryo to develop.

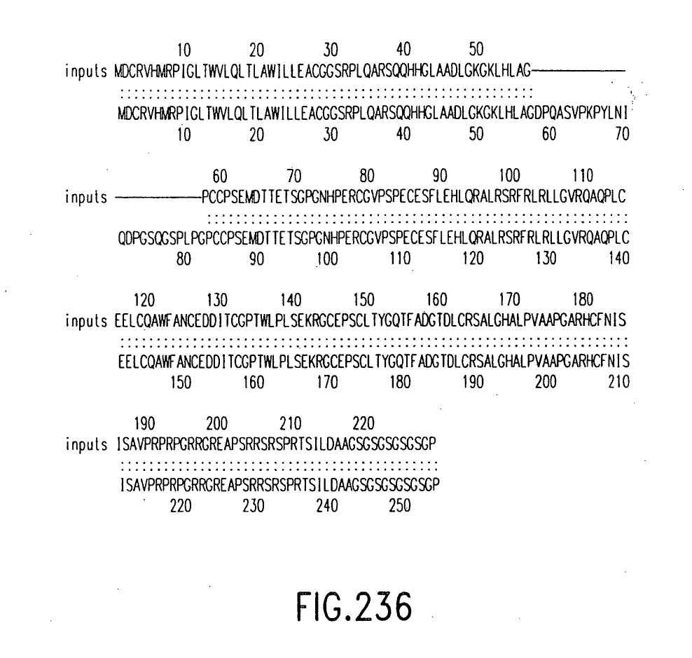

FIG. 236 depicts an alignment of TANGO 499 form 1, variant 1 amino acid sequence and TANGO 499 form 2, variant 3 amino acid sequence. The amino acid sequences are 90.2% identical, the cDNAs are 85.6% identical, and the ORFs are 90.2% identical. The alignment clearly demonstrates that the two forms are identical except for the result of a putative alternative exon splicing event in the illustrated region. The invention contemplates additional splice variants of the presently claimed nucleic acids and proteins encoded by such splice variants.

Clone EpT499, which encodes TANGO 499 form 2, variant 3, was deposited with the American Type Culture Collection (10801 University Boulevard, Manassas, Va. 20110-2209) on Aug. 5, 1999 and assigned Accession Number PTA-454. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Additional Human TANGO 499 Variants

Analysis of multiple individual human TANGO 499 clones revealed a complex set of variant transcripts (e.g., alternatively spliced transcripts). One such alternatively spliced form of a human TANGO 499 gene is referred to as a human TANGO 499 form 1, variant 1, which is also described in detail above. Another such alternatively spliced form of a human TANGO 499 gene is referred to as a human TANGO 499 form 2, variant 3, which is described in detail above. Additional human TANGO 499 form 1 and form 2 variants have been identified and are described below.

In one embodiment, the open reading frame of the form 1 TANGO 499 cDNA conserved region comprises nucleotides 1 to 783 of the TANGO 499 open reading frame.

In another embodiment, the open reading frame of the form 2 TANGO 499 cDNA conserved region comprises nucleotides 1 to 708 of the TANGO 499 open reading frame.

In another embodiment, the open reading frame of a TANGO 499 cDNA comprises nucleotides 26 to 847 of the TANGO 499 open reading frame and encodes a polypeptide referred to herein as form 2, variant 1.

In another embodiment, the open reading frame of a TANGO 499 cDNA comprises nucleotides 11 to 794, and encodes a polypeptide comprising the sequence of the TANGO 499 open reading frame referred to herein as form 1, variant 2.

In another embodiment, the open reading frame of this cDNA comprises nucleotides 11 to 719, and encodes a polypeptide comprising the sequence of the TANGO 499 open reading frame referred to herein as form 2, variant 2.

In another embodiment, the open reading frame of a TANGO 499 cDNA comprises nucleotides 447 to 1230, and encodes a polypeptide comprising the sequence of the TANGO 499 open reading frame referred to herein as form 1, variant 3.

In another embodiment, the open reading frame of this cDNA comprises nucleotides 95 to 908, and encodes a polypeptide comprising the sequence of the TANGO 499 open reading frame referred to herein as form 1, variant 4.

In another embodiment, the open reading frame of this TANGO 499 comprises nucleotides 95 to 833, and encodes a polypeptide comprising the sequence of the TANGO 499 open reading frame referred to herein as form 2, variant 4.

### Uses of TANGO 499 Nucleic Acids, Polypeptides, and Modulators Thereof

As TANGO 499 was originally found in a human pituitary library, TANGO 499 nucleic acids, proteins, and modulators thereof can be used to modulate the proliferation, development, differentiation, and/or function of cells, tissues and/or organs, e.g., the proliferation of tissues and cells of pituitary origin.

Thus, as TANGO 499 was originally identified in a pituitary library, for example, TANGO 499 nucleic acids, proteins and modulators thereof can be used to regulate processes involved with sexual development and function, including, e.g., normal and abnormal reproductive hormonal function in the fetus, infant, adolescent and adult and also can modulate the effects of pituitary insufficiency and pituitary adenomas on sexual development, reproductive function and sexuality in men and women. Furthermore, TANGO 499 nucleic acids, proteins and modulators thereof can be used to treat pituitary tumors causing Cushing's syndrome, and also hypopituitarism during pregnancy which may be the result of intrasellar adenomas, suprasellar lesions, lymphocytic hypophysitis or antepartum pituitary necrosis, and in the postpartum period may be because of postpartum hemorrhage and pituitary necrosis. TANGO 499 nucleic acids, proteins and modulators thereof can also be used to treat posterior pituitary problems in pregnancy manifested by diabetes insipidus, with a pregnancy-specific variety resulting from excessive degradation of arginine vasopressin by placental vasopressinase. Further, TANGO 499 nucleic acids, proteins and modulators thereof can be used to treat pituitary-related disorders, e.g., hormone secretion disorders, (e.g., Cushing's disease, hyperprolactinemia, acromegaly-gigantism, and precocious and delayed puberty).

In light of the fact that TANGO 499 was identified in a retina library, TANGO 499 nucleic acids, proteins and modulators thereof can be utilized to modulate the development and function of the eye, such as retinal development and function, (e.g., photoreceptor disk morphogenesis). TANGO 499 nucleic acids, proteins and modulators thereof can be utilized to treat eye diseases and/or disorders, e.g., autosomal dominant retinitis pigmentosa, autosomal dominant punctata albescens, butterfly-shaped pigment dystrophy, cataracts, macular degeneration, myopia, stigmatism and retinoblastoma.

TANGO 499 family members have homology to glial cell line-derived neurotrophic-related factors (e.g., GDNF, artemin, neurturin, and persephin). Thus, TANGO 499 nucleic acids, proteins and modulators thereof can be utilized to modulate survival, activation, proliferation, motility, and differentiation of peripheral or central neurons.

TANGO 499 nucleic acids, proteins and modulators thereof can be used to modulate kidney development or for gene therapy for modulating defects in kidney organogenesis. As such, TANGO 499 nucleic acids, proteins and modulators thereof can also be used to modulate renal disorders, e.g., glomerular disease, (e.g., acute and chronic glomerulonephritis), tubular diseases, and tubulo-interstitial diseases.

TANGO 499 nucleic acids, proteins and modulators thereof can also be used to modulate intercellular signaling in the nervous system, to modulate disorders associated with aberrant signal transduction in response to neurotrophic factors and cell surface receptors such as, e.g., other GDNF proteins, and to modulate myelin-associated processes. For example, TANGO nucleic acids, proteins and modulators thereof can be used to maintain the myelin sheath e.g., enhance myelin membrane adhesion to extracellular matrices during development, e.g., at late stages of development.

Furthermore, TANGO 499 nucleic acids, proteins, and modulators thereof can be used to modulate the proliferation, development, differentiation, and/or function of neural organs, e.g., neural tissues and cells, e.g., cells of the central nervous system, e.g., cells of the peripheral nervous system. TANGO 499 nucleic acids, proteins, and modulators thereof can also be used to modulate symptoms associated with abnormal neural signaling and function, e.g., epilepsy, stroke, traumatic injury. In particular, TANGO 499 proteins could be useful to treat neural related disorders or neural damage, such as for regenerative neural repair after damage by trauma, degeneration, or inflammation e.g., spinal cord injuries, infarction, infection, malignancy, exposure to toxic agents, nutritional deficiency, paraneo-plastic syndromes, and degenerative nerve diseases including but not limited to Alzheimer's disease, Parkinson's disease, Huntington's Chorea, amyotrophic lateral sclerosis, progressive supra-nuclear palsy, Hirchsprung's disease, and other dementias or peripheral neuropathy-related disorders.

maintenance of the entire myelin sheath.

As TANGO 499 family members have homology to riboflavin binding proteins, TANGO 499 nucleic acids, proteins and modulators thereof can be used to modulate cofactor or vitamin concentrations, in particular TANGO 499 nucleic acids, proteins and modulators thereof can be used to modulate vitamin concentrations in and around an embryo, to modulate the development of an embryo, and to modulate the length of time of the pregnancy (i.e., terminating).

Moreover, TANGO 499 nucleic acids, proteins and modulators thereof can be utilized to modulate the development and progression of cancerous- and non-cancerous cell proliferative disorders, such as deregulated proliferation (such as hyperdysplasia, hyper-IgM syndrome, or lymphoproliferative disorders), cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, and tissue hypertrophy (e.g., prostatic hyperplasia), cancers such as neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias, (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), or polycythemia vera, or lymphomas (Hodgkin's disease and non-Hodgkin's diseases), multiple myelomas and Waldenstrom's macroglobulinemia.

TANGO 499 expression can be utilized as a marker (e.g., an in situ marker) for specific tissues (e.g., the pituitary) and/or cells (e.g., pituitary cells) in which TANGO 499 is expressed. TANGO 499 nucleic acids can also be utilized for chromosomal mapping, or as chromosomal markers, e.g., in radiation hybrid mapping.

### TANGO 315, TANGO 330, TANGO 437 and TANGO 480

The TANGO 315, TANGO 330, TANGO 437 and TANGO 480 proteins and nucleic acid molecules comprise families of molecules having certain conserved structural and functional features.

For example, the TANGO 315, TANGO 330, TANGO 437 and TANGO 480 proteins of the invention can have signal sequences.

Thus, in one embodiment, a TANGO 315 form 2 protein can contain a signal sequence of about amino acids 1 to 26. In another embodiment, a TANGO 330 protein can contain a signal sequence of about amino acids 1 to 20. In another embodiment, a TANGO 480 protein can contain a signal sequence of about 1 to 19. In one embodiment, a TANGO 315 family member is a polypeptide comprising the amino acid sequence. In another embodiment, a TANGO 315 family member is a polypeptide comprising the amino acid sequence of SEQ ID NO:152.

A TANGO 315 family member can include one or more of the following domains: (1) an extracellular domain; (2) a transmembrane domain; and (3) a cytoplasmic domain. In one embodiment, a TANGO 315 form 1 protein contains an extracellular domain at about amino acid residues 46 to 251, two transmembrane domains at about amino acid residues 29 to 45 and at about amino acid residues 252 to 276, and two cytoplasmic domains at about amino acid residues 1 to 28 and at about amino acid residues 277 to 296 of SEQ ID NO: 152.

In another embodiment, a TANGO 315 form 1 protein comprises an extracellular domain comprising amino acid residues 1 to 251, a transmembrane domain comprising amino acid residues 252 to 276 and a cytoplasmic domain comprising amino acid residues 277 to 296. In this embodiment, therefore, TANGO 315 protein comprises amino acids 1 to 296 of SEQ ID NO:152.

In another embodiment, a TANGO 315 form 2 protein comprises an extracellular domain at about amino acid residues 27 to 232, a transmembrane domain at about amino acid residues 233 to 257 and a cytoplasmic domain at about amino acid residues 258 to 277. In this embodiment, the mature TANGO 315 form 2 protein corresponds to amino acids 27 to 277 of SEQ ID NO:154.

A TANGO 315 family member can include a signal sequence. In certain embodiments, a TANGO 315 family member has the amino acid sequence of SEQ ID NO:154, and the signal sequence is located at amino acids 1 to 24, 1 to 25, 1 to 26, 1 to 27 or 1 to 28. In such embodiments of the invention, the domains and the mature protein resulting from cleavage of such signal peptides are also included herein. For example, the cleavage of a signal sequence consisting of amino acids 1 to 26 results in an extracellular domain consisting of amino acids 27 to 232 and the mature TANGO 315 form 2 protein corresponding to amino acids 27 to 277 of SEQ ID NO: 154.

A TANGO 315 family member can include one or more TANGO 315 Ig-like domains. A TANGO 315 Ig-like domain as described herein is about 58 amino acid residues in length and has the following consensus sequence, beginning about 1 to 15 amino acid residues, more preferably about 3 to 10 amino acid residues, and most preferably about 5 amino acid residues from the domain C-terminus: [FYL]-Xaa-C-Xaa-[VA], wherein [FYL] is a phenylalanine, tyrosine or leucine residue (preferably tyrosine), where “Xaa” is any amino acid, C is a cysteine residue, and A is a alanine and V is a valine residue. In one embodiment, a TANGO 315 family member includes one or more Ig-like domains having an amino acid sequence that is at least about 55%, preferably at least about 65%, more preferably at least about 75%, yet more preferably at least about 85%, and most preferably at least about 95% identical to amino acids 151 to 209. In another embodiment, a TANGO 315 family member includes one or more Ig-like domains having an amino acid sequence that is at least about 55%, preferably at least about 65%, more preferably at least about 75%, yet more preferably at least about 85%, and most preferably at least about 95% identical to amino acids 132 to 190.

In another embodiment, a TANGO 315 family member includes one or more TANGO 315 Ig-like domains having an amino acid sequence that is at least about 55%, preferably at least about 65%, more preferably at least about 75%, yet more preferably at least about 85%, and most preferably at least about 95% identical to amino acids. 151 to 209, and has a conserved cysteine residue about 8 residues downstream from the N-terminus of the Ig-like domain. Thus, in this embodiment, amino acid 158 is a cysteine residue. In another embodiment, a TANGO 315 family member includes one or more TANGO 315 Ig-like domains having an amino acid sequence that is at least about 55%, preferably at least about 65%, more preferably at least about 75%, yet more preferably at least about 85%, and most preferably at least about 95% identical to amino acids 132 to 190, and has a conserved cysteine residue about 8 residues downstream from the N-terminus of the Ig-like domain. Thus, in this embodiment, amino acid 139 is a cysteine residue.

In another embodiment, a TANGO 315 family member includes one or more TANGO 315 Ig-like domains having an amino acid sequence that is at least 55%, preferably at least about 65%, more preferably at least about 75%, yet more preferably at least about 85%, and most preferably at least about 95% identical to amino acids 151 to 209, and has a conserved cysteine residue about 8 residues downstream from the N-terminus of the TANGO 315 Ig-like domain, has a conserved cysteine within the consensus sequence that forms a disulfide with said first conserved cysteine, and has at least one TANGO 315 biological activity as described herein. In yet another embodiment, a TANGO 315 family member includes one or more TANGO 315 Ig-like domains having an amino acid sequence that is at least 55%, preferably at least about 65%, more preferably at least about 75%, yet more preferably at least about 85%, and most preferably at least about 95% identical to amino acids 132 to 190, and has a conserved cysteine residue about 8 residues downstream from the N-terminus of the TANGO 315 Ig-like domain, has a conserved cysteine within the consensus sequence that forms a disulfide with said first conserved cysteine, and has at least one TANGO 315 biological activity as described herein.

In another embodiment, the Ig-like domain of TANGO 315 is an Ig domain. An Ig domain as used in the context of TANGO 315 is about 58 amino acid residues in length and has the following consensus sequence, beginning at about 1 to 15 amino acid residues, more preferably about 3 to 10 amino acid residues, and most preferably about 5 amino acid residues from the C-terminal end of the domain: [FY]-Xaa-C-Xaa-[VA]-Xaa-H—COO—, wherein [FY] is either a phenylalanine or a tyrosine residue (preferably tyrosine), where “Xaa” is any amino acid, C is a cysteine residue, [VA] is either valine or an alanine residue (preferably alanine), His a histidine residue and COO— is the C-terminus of the domain. In this embodiment, a TANGO 315 family member includes one or more Ig-like domains having an amino acid sequence that is at least about 55%, preferably at least about 65%, more preferably at least about 75%, yet more preferably at least about 85%, and most preferably at least about 95% identical to amino acids 151 to 209 and/or amino acids 132 to 190.

A TANGO 330 form 1 protein is encoded by a nucleic acid sequence comprising nucleotides 1 to 3042 (SEQ ID NO:151). In another embodiment, a TANGO 330 form 1 has an open reading frame comprised of nucleotides 2 to 2808 of SEQ ID NO:152. In another embodiment, a TANGO 330 form 1 protein is a polypeptide comprising the amino acid sequence at amino acids 1 to 934 (SEQ ID NO:152). A TANGO 330 form 2 protein is encoded by a nucleic acid sequence comprising nucleotides 1 to 3808 (SEQ ID NO: 153). In another embodiment, a TANGO 330 form 2 cDNA has an open reading frame comprised of nucleotides 9 to 1448 of SEQ ID NO:153. In another embodiment, a TANGO 330 form 2 protein is a polypeptide comprising the amino acid sequence at amino acids 1 to 480 (SEQ ID NO:154).

A TANGO 330 family member can include one or more of the following domains: 35(1) an extracellular domain; (2) a transmembrane domain; and (3) a cytoplasmic domain. In one embodiment, a TANGO 330 form 1 protein comprises extracellular domains comprising amino acid residues 1 to 393, a transmembrane domain comprising amino acid residues 394 to 417 and a cytoplasmic domain comprising amino acid residues 418 to 934 of SEQ ID NO:156. In this embodiment, therefore TANGO 330 protein comprises amino acids 1 to 934 (SEQ ID NO:156).

A TANGO 330 family member can include a signal sequence. In certain embodiments, a TANGO 330 family member has the amino acid sequence, and the signal sequence is located at amino acids 1 to 18, 1 to 19, 1 to 21 or 1 to 22. In such embodiments of the invention, the domains and the mature protein resulting from cleavage of such signal peptides are also included herein. For example, the cleavage of the signal sequence of TANGO 330 form 2 at amino acids 1 to 20 results in a mature protein comprising amino acids 21 to 480 (SEQ ID NO:158).

A TANGO 330 family member can include one or more fibronectin type II-like domains. The nucleotide sequence of a typical fibronectin type II domain is disclosed in Pfam Accession Number PF00041. In one embodiment, a TANGO 330 family member includes one or more fibronectin type II-like domains having an amino acid sequence that is at least about 55%, preferably at least about 65%, more preferably at least about 75%, yet more preferably at least about 85%, and most preferably at least about 95% identical to TANGO 330 form 1 at amino acids 179 to 262 and amino acids 274 to 359, or alternatively, to TANGO 330 form 2 at amino acids 283 to 366 and amino acids 378 to 463 of SEQ ID NO:158.

In another embodiment, a TANGO 330 family member includes one or more fibronectin type II-like domains having an amino acid sequence that is at least about 55%, preferably at least about 65%, more preferably at least about 75%, yet more preferably at least about 85%, and most preferably at least about 95% identical to amino acids 179 to 262 and amino acids 274 to 359, or alternatively to amino acids 283 to 366 and amino acids 378 to 463, and has at least one TANGO 330 biological activity as described herein.

In another embodiment, a TANGO 330 family member includes one or more fibronectin type II domains having an amino acid sequence that is at least about 55%, preferably at least about 65%, more preferably at least about 75%, yet more preferably at least about 85%, and most preferably at least about 95% identical to TANGO 330 form 1 at amino acids 179 to 262 and amino acids 274 to 359, or alternatively, to TANGO 330 form 2 at amino acids 283 to 366 and amino acids 378 to 463. In another embodiment, a TANGO 330 family member includes one or more fibronectin type II domains having an amino acid sequence that is at least about 55%, preferably at least about 65%, more preferably at least about 75%, yet more preferably at least about 85%, and most preferably at least about 95% identical to amino acids 179 to 262 and amino acids 274 to 359, or alternatively to amino acids 283 to 366 and amino acids 378 to 463, and has at least one TANGO 330 biological activity as described herein.

A TANGO 330 family member can include one or more Ig-like domains. A TANGO 330 Ig-like domain as described herein has the following consensus sequence, beginning about 1 to 15 amino acid residues, more preferably about 3 to 10 amino acid residues, and most preferably about 5 amino acid residues from the domain C-terminus: [FY]-Xaa-C-Xaa-[VA], wherein [FY] is either a phenylalanine or a tyrosine residue (preferably tyrosine), where “Xaa” is any amino acid, C is a cysteine residue and [VA] is either a valine or alanine residue. In one embodiment, a TANGO 330 family member includes one or more Ig-like domains having an amino acid sequence that is at least about 55%, preferably at least about 65%, more preferably at least about 75%, yet more preferably at least about 85%, and most preferably at least about 95% identical to amino acids 78 to 136, or amino acids 77 to 147, or amino acids 182 to 240.

In one embodiment, a TANGO 330 family member includes one or more Ig-like domains having an amino acid sequence that is at least about 55%, preferably at least about 65%, more preferably at least about 75%, yet more preferably at least about 85%, and most preferably at least about 95% identical to amino acids 78 to 136, or amino acids 77 to 147, or amino acids 182 to 240 and has a conserved cysteine residue about 8 residues downstream from the N-terminus of the Ig-like domain.

In another embodiment, a TANGO 330 family member includes one or more TANGO 330 Ig-like domains having an amino acid sequence that is at least about 55%, preferably at least about 65%, more preferably at least about 75%, yet more preferably at least about 85%, and most preferably at least about 95% identical to amino acids 78 to 136, or amino acids 77 to 147, or amino acids 182 to 240 and has a conserved cysteine residue about 8 residues downstream from the N-terminus of the Ig-like domain. Thus, in this embodiment, the amino residue corresponding to amino acid 85, or to amino acid 84 to amino acid 189.

In yet another embodiment, a TANGO 330 family member includes one or more TANGO 330 Ig-like domains having an amino acid sequence that is at least 55%, preferably at least about 65%, more preferably at least about 75%, yet more preferably at least about 85%, and most preferably at least about 95% identical to amino acids 78 to 136, or amino acids 77 to 147, or amino acids 182 to 240, and has a conserved cysteine residue about 8 residues downstream from the N-terminus of the Ig-like domain, has a conserved cysteine within the consensus sequence that forms a disulfide with said first conserved cysteine, and has at least one TANGO 330 biological activity as described herein.

In another embodiment, the Ig-like domain of TANGO 330 is an Ig domain. In this embodiment, a TANGO 330 family member includes one or more Ig-like domains having an amino acid sequence that is at least about 55%, preferably at least about 65%, more preferably at least about 75%, yet more preferably at least about 85%, and most preferably at least about 95% identical to amino acids 78 to 136, or amino acids 77 to 147, or amino acids 182 to 240.

A TANGO 437 family member can include one or more of the following domains: (1) an extracellular domain; (2) a transmembrane domain; and (3) a cytoplasmic domain.

In one embodiment, a TANGO 437 protein contains extracellular domains at about amino acid residues 1 to 84, 150 to 155, 241 to 287, 456 to 466, and 524 to 591, transmembrane domains at about amino acid residues 85 to 101, 130 to 149, 156 to 180, 216 to 240, 288 to 312, 436 to 455, 467 to 486, and 506 to 523, and cytoplasmic domains at about amino acid residues 102 to 129, 181 to 215, 313 to 435, and 487 to 505 of SEQ ID NO:160.

In another embodiment, a TANGO 437 protein contains extracellular domains at about amino acid residues 1 to 84, 181 to 215, 313 to 435, and 487 to 505, the following seven transmembrane domains at about amino acid residues 85 to 101, 156 to 180, 216 to 240, 288 to 312, 436 to 455, 467 to 486, and 506 to 523, and cytoplasmic domains at about amino acid residues 102 to 155, 241 to 287, 456 to 466, 524 to 591. In these embodiments, the mature TANGO 437 protein corresponds to amino acids 1 to 591 (SEQ ID NO:160).

In another embodiment, a TANGO 437-form 2 protein contains extracellular domains at about amino acid residues 1 to 84, 181 to 215, 313 to 435, 524 to 580, and 656 to 671, transmembrane domains at about amino acid residues 85 to 101, 130 to 149, 156 to 180, 216 to 240, 288 to 312, 436 to 455, 467 to 486, 506 to 523, 581 to 601, 639 to 655, and 672 to 694, and cytoplasmic domains at about amino acid residues 102 to 155, 241 to 287, 456 to 505, 602 to 638, and 695 to 752 (SEQ ID NO: 164).

A TANGO 437 family member can include one or more ion transport protein-like domains. The nucleotide sequence of a typical ion transport protein domain is disclosed in Pfam Accession Number PF00520. A TANGO 437 ion transport protein-like domain as described herein has the following consensus sequence: [L]-[R]-Xaa-Xaa-[R]-Xaa-[L]-[R]-Xaa(n1)-[L]-Xaa(n2)-[S]-Xaa(n3)-[L]-[L], wherein [L] is a leucine residue, [R] is arginine, Xaa is any amino acid, n1 is about 1 to 10, preferably 2 to 7, more preferably 3, n2 is about 1 to 15, more preferably about 8 to 20, more preferably about 16, [S] is serine, and n3 is about 1 to 15, preferably about 5 to 11, more preferably about 8. In one embodiment, a TANGO 437 family member includes one or more ion transport protein-like domains having an amino acid sequence that is at least about 55%, preferably at least about 65%, more preferably at least about 75%, yet more preferably at least about 85%, and most preferably at least about 95% identical to amino acids 82 to 311. In another embodiment, a TANGO 437 family member includes one or more TANGO 437 ion transport protein-like domains having an amino acid sequence that is at least 55%, preferably at least about 65%, more preferably at least about 75%, yet more preferably at least about 85%, and most preferably at least about 95% identical to amino acids 82 to 311, and has at least one TANGO 437 biological activity as described herein.

A TANGO 437 family member can include one or more putative permease domains. The nucleotide sequence of a typical putative permease domain is disclosed in Pfam Accession Number PF01594. A TANGO 437 putative permease-like domain as described has the following consensus sequence: [P]-Xaa(n1)-[S]-Xaa(3)-[G]-Xaa(n2)-[F]-[G]-Xaa(n3)-[G]-Xaa(4)-[P], wherein P is a proline residue, Xaa is any amino acid, n1 is about 1 to 10, preferably about 3 to 8, more preferably about 5, S is serine, G is glycine, n2 is about 1 to 15, preferably about 2 to 10, more preferably about 3 to 7, F is phenylalanine, and n3 is about 0 to 5, more preferably about 0 to 2. In one embodiment, a TANGO 437 family member includes one or more putative permease domains having an amino acid sequence that is at least about 55%, preferably at least about 65%, more preferably at least about 75%, yet more preferably at least about 85%, and most preferably at least about 95% identical to amino acids 284 to 591. In another embodiment, a TANGO 437 family member includes one or more TANGO 437 putative permease domains having an amino acid sequence that is at least 55%, preferably at least about 65%, more preferably at least about 75%, yet more preferably at least about 85%, and most preferably at least about 95% identical to amino acids 284 to 591, and has at least one TANGO 437 biological activity as described herein.

A TANGO 480 family member can include one or more of the following domains: (1) an extracellular domain; (2) a transmembrane domain; and (3) a cytoplasmic domain. In one embodiment, a TANGO 480 protein is a transmembrane protein that contains extracellular domains at about amino acid residues 20 to 56 and 113 to 127, transmembrane domains at about amino acid residues 57 to 74, 88 to 112, and 128 to 150, and cytoplasmic domains at about amino acid residues 75 to 87 and 151 to 193 of SEQ ID NO: 162.

A TANGO 480 family member can include a signal sequence. In certain embodiments, a TANGO 480 family member has the amino acid sequence, and the signal sequence is located at amino acids 1 to 17, 1 to 18, 1 to 19, 1 to 20 or 1 to 21. In such embodiments of the invention, the domains and the mature protein resulting from cleavage of such signal peptides are also included herein. For example, the cleavage of a signal sequence consisting of amino acids 1 to 19 results in an extracellular domain consisting of amino acids 20 to 56 and a mature TANGO 480 protein corresponding to amino acids 20 to 193 of SEQ ID NO:162.

### Human TANGO 315

A cDNA encoding human TANGO 315 was identified by analyzing the sequences of clones present in a human natural killer cell library for sequences that encode wholly secreted or transmembrane proteins. This analysis led to the identification of a clone, jthta123e06, encoding human TANGO 315.

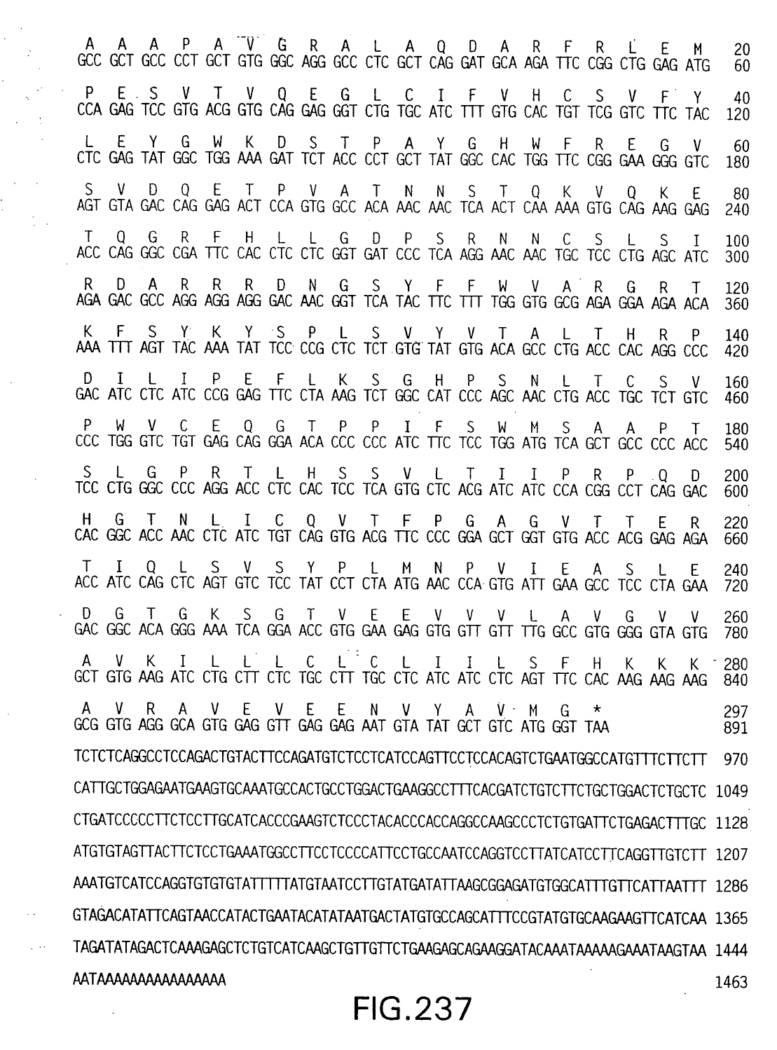

The human TANGO 315 cDNA of this clone is 1463 nucleotides long (FIG. 237; SEQ ID NO:151). In one embodiment, TANGO 315 is referred to as TANGO 315, form 1. The open reading frame of TANGO 315 form 1 comprises nucleotides 1 to 888, and encodes a transmembrane protein comprising the 296 amino acid sequence depicted in SEQ ID NO: 152. The protein has a predicted molecular weight of 32.6 kDa without post-translational modification.

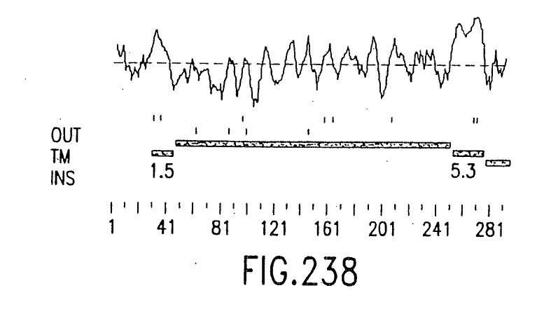

FIG. 238 depicts a hydropathy plot of the human TANGO 315 form 1 amino acid sequence depicted in FIG. 237.

Human TANGO 315 form 1 protein is a transmembrane protein comprising amino acids 1 to 296. In particular, human TANGO 315, form 1 protein contains an extracellular domain comprising at amino acid residues 1 to 251, a transmembrane domain comprising amino acid residues 252 to 276 and a cytoplasmic domain comprising amino acid residues 277 to 296 of SEQ ID NO:152.

Alternatively, in another embodiment, a human TANGO 315 protein is a transmembrane protein that contains a cytoplasmic domain comprising amino acid residues 1 to 251, a transmembrane domain comprising amino acid residues 252 to 276 and an extracellular domain comprising amino acid residues 277 to 296 of SEQ ID NO: 152.

In one embodiment a cDNA sequence of human TANGO 315 has a nucleotide at position 66 which is guanine (G). In this embodiment, the cDNA contains an open reading frame encoding a polypeptide having an amino acid at position 22 that is glutamate (E). In an alternative embodiment, a species variant cDNA sequence of human TANGO 315 has a nucleotide at position 66 which is cytosine (C). In this embodiment, the cDNA contains an open reading frame encoding a polypeptide having an amino acid at position 22 that is aspartate (E), i.e., a conservative substitution.

In another embodiment a cDNA sequence of human TANGO 315 has a nucleotide at position 67 which is thymidine (T). In this embodiment, the cDNA contains an open reading frame encoding a polypeptide having an amino acid at position 23 that is serine (S). In an alternative embodiment, a species variant cDNA sequence of human TANGO 315 has a nucleotide at position 67 which is adenine (A). In this embodiment, the cDNA contains an open reading frame encoding a polypeptide having an amino acid at position 23 that is threonine (T), i.e., a conservative substitution.

In another embodiment a cDNA sequence of human TANGO 315 has a nucleotide at position 70 which is guanine (G). In this embodiment, the cDNA contains an open reading frame encoding a polypeptide having an amino acid at position 24 that is valine (V). In an alternative embodiment, a species variant cDNA sequence of human TANGO 315 has a nucleotide at position 70 which is cytosine (C). In this embodiment, the cDNA contains an open reading frame encoding a polypeptide having an amino acid at position 24 that is leucine (L), i.e., a conservative substitution.

In another embodiment a cDNA sequence of human TANGO 315 has a nucleotide at position 138 which is adenine (A). In this embodiment, the cDNA contains an open reading frame encoding a polypeptide having an amino acid at position 46 that is lysine (K). In an alternative embodiment, a species variant cDNA sequence of human TANGO 315 has a nucleotide at position 138 which is guanine (G). In this embodiment, the cDNA contains an open reading frame encoding a polypeptide having an amino acid at position 46 that is arginine (R), i.e., a conservative substitution.

Human TANGO 315 form 1 includes an Ig-like domain at amino acids 151 to 209 of SEQ ID NO: 152.

Four N-glycosylation sites are present in TANGO 315 form 1. The first has the sequence NNST (at amino acid residues 71 to 74), the second has the sequence NCSL (at amino acid residues 95 to 98), the third has the sequence NGSY (at amino acid residues 108 to 111), and the fourth has the sequence NLTC (at amino acid residues 155 to 158). Six protein kinase C phosphorylation sites are present in TANGO 315 form 1. The first has the sequence TQK (at amino acid residues 74 to 76), the second has the sequence SIR (at amino acid residues 99 to 101), the third has the sequence SYK (at amino acid residues 123 to 125), the fourth has the sequence THR (at amino acid residues 137 to 139), the fifth has the sequence TER (at amino acid residues 218 to 220), and the sixth has the sequence TGK (at amino acid residues 243 to 245). TANGO 315 form 1 has four casein kinase II phosphorylation sites. The first has the sequence TVQE (at amino acid residues 25 to 28), the second has the sequence SIRD (at amino acid residues 99 to 102), the third has the sequence SLED (at amino acid residues 238 to 241), and the fourth has the sequence TVEE (at amino acid residues 248 to 251). TANGO 315 form 1 has one tyrosine kinase phosphorylation site with the sequence RRRDNGSY at amino acid residues 104 to 111. Two N-myristylation sites are present in TANGO 315 form 1. The first has the sequence GAGVTT (at amino acid residues 213 to 218) and the second has the sequence GTGKSG (at amino acid residues 242 to 247).

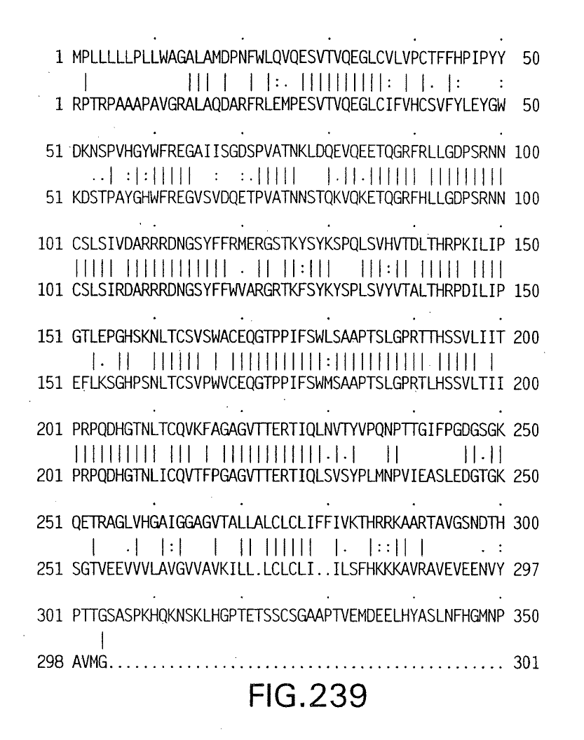

FIG. 239 depicts an alignment of the amino acid sequence of human TANGO 315 form 1 and the amino acid sequence of CD33 (Accession Number NP_{—}001763). The alignment shows that there is a 59.4% overall amino acid sequence identity between TANGO 315 form 1 and CD33. CD33 is an early or immature marker expressed by myeloid cells. The expression of CD33 has been shown to be associated with the development and/or progression of myelodysplastic syndrome (MDS) and acute myelogenous leukemia (AML) (Eghetany, 1998, Haematologica 83: 1104-1115; Matthews, 1998, Leukemia 12 Suppl. 1:S33-36). As such, TANGO 315, nucleic acids and proteins may be useful, for example, as early markers for the development of MDS and AML.

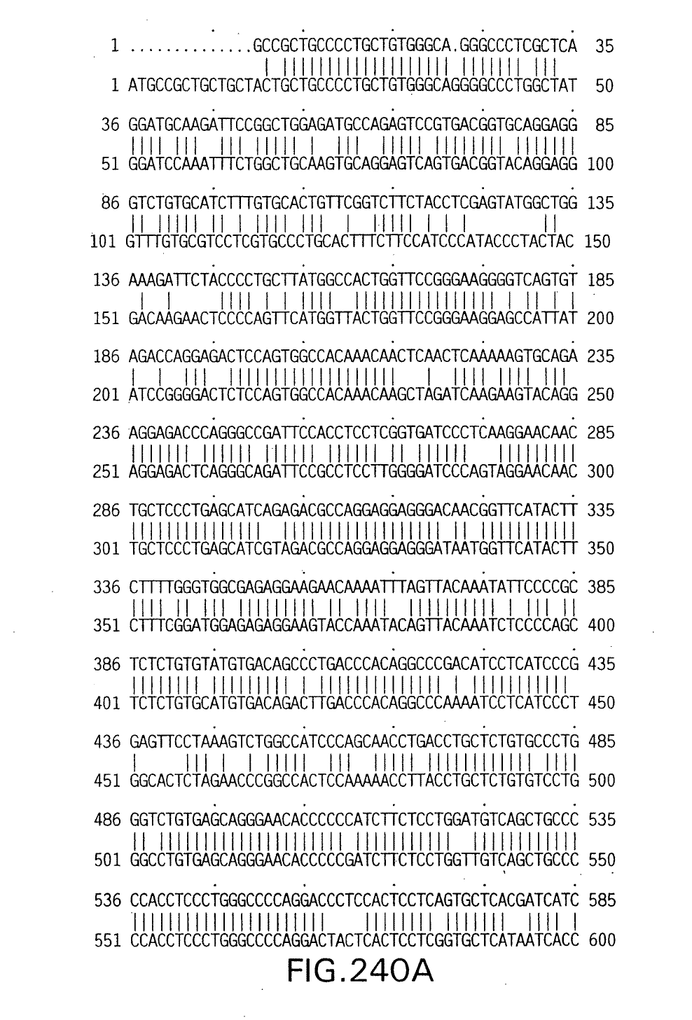

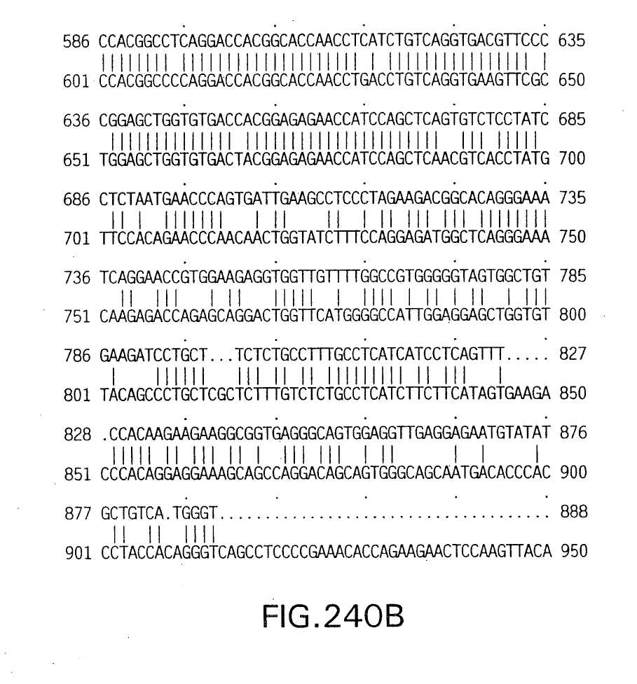

FIG. 240A-240B depicts an alignment of the nucleotide sequence of the coding region of CD33 (Accession Number NM_{—}001772) and the nucleotide sequence of the coding region of human TANGO 315 form 1. The nucleotide sequences of the coding regions of CD33 and human TANGO 315 form 1 are 75.8% identical. The nucleic acid sequence of CD33 (Accession Number NM_{—}001772) and the nucleic acid of sequence of human TANGO 315 form 1 are 67.7% identical.

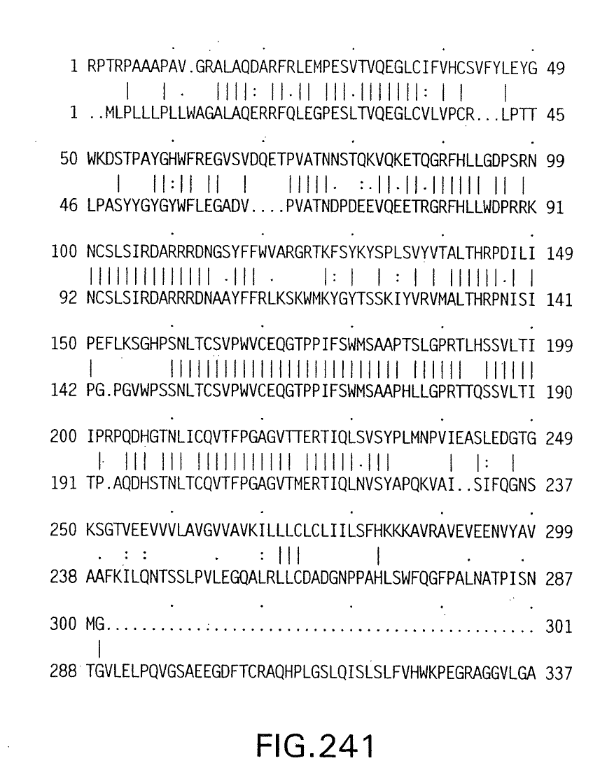

FIG. 241 depicts an alignment of the amino acid sequence of TANGO 315 form 1 and the amino acid sequence of Ob binding protein (Accession Number AAB70702). The alignment shows that there is a 52.8% overall amino acid sequence identity between TANGO 315 form 1 and OB-BP-1. OB-BP-1, like CD33, is a member of the sialic acid-binding immunoglobulin superfamily (Siglec) which binds to Leptin (Patel et al., 1999, J. Biol. Chem. 274:22729-22738). Leptin plays a role in the regulation of neuroendocrine function and the energy metabolism of adipocytes and skeletal muscle (Fruhbeck et al, 1998, Clin. Physiol. 18:399-419). As such, TANGO 315, nucleic acids, proteins and modulators thereof may be useful, for example, to modulate the development of obesity, anorexia nervosa, diabetes mellitus, polycystic ovary syndrome, acquired immunodeficiency syndrome, cancer, nephropathy, thyroid disease, Cushing's syndrome, and growth hormone deficiency.

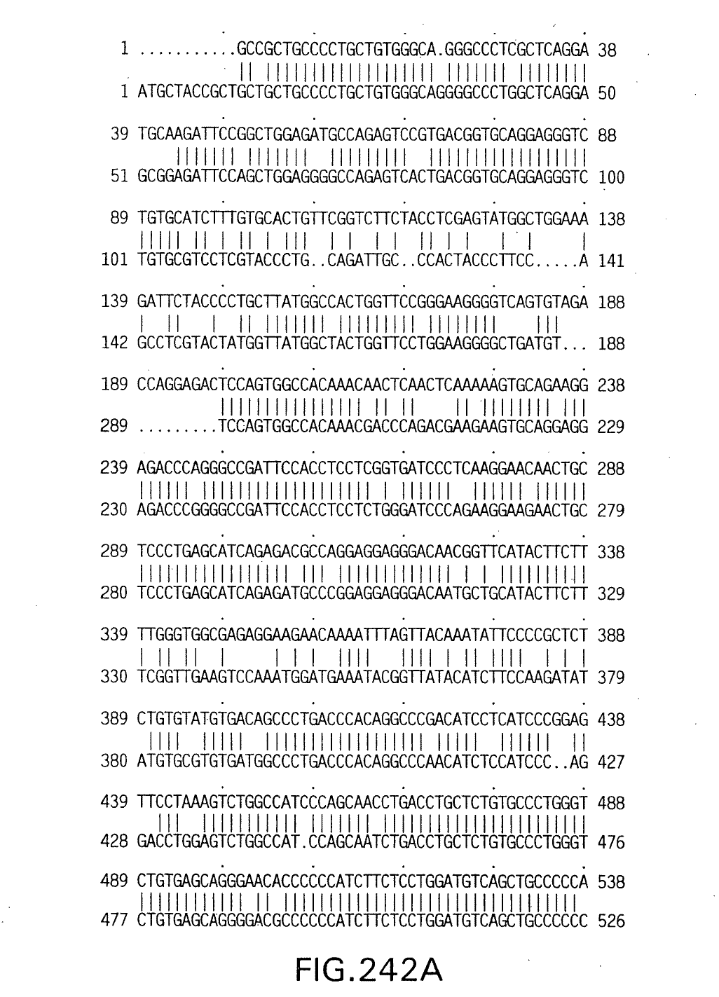

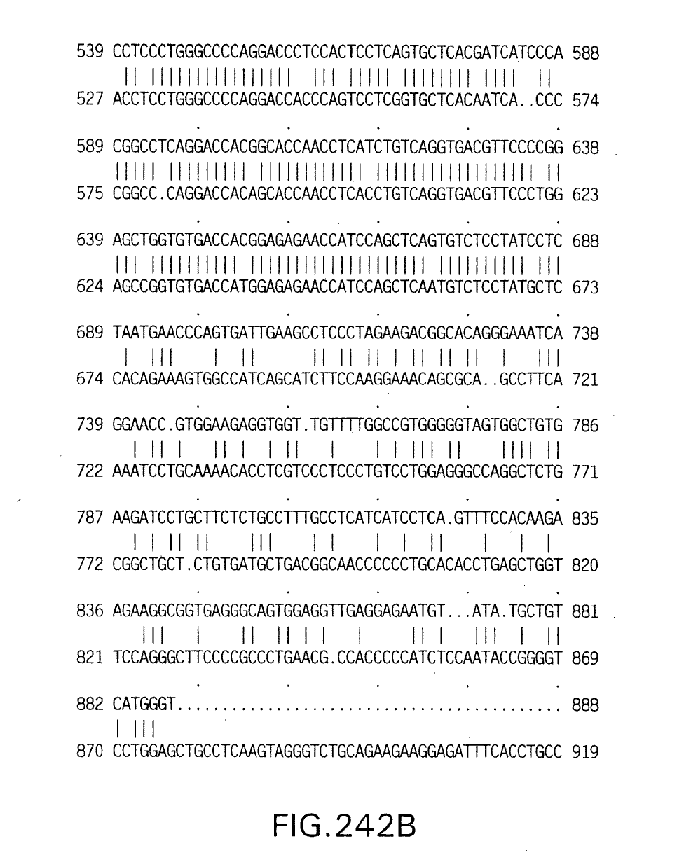

FIG. 242A-242B depicts an alignment of the nucleotide sequence of human TANGO 315 form 1 coding region and the nucleotide sequence of human OB-BP-1 coding region (Accession Number U71382). The nucleotide sequences of the coding regions are 74.2% identical. The nucleotide sequence of the TANGO 315 form 1 nucleic acid sequence and human OB-BP-1 cDNA (Accession Number U71382) have an overall sequence identity of 65%.

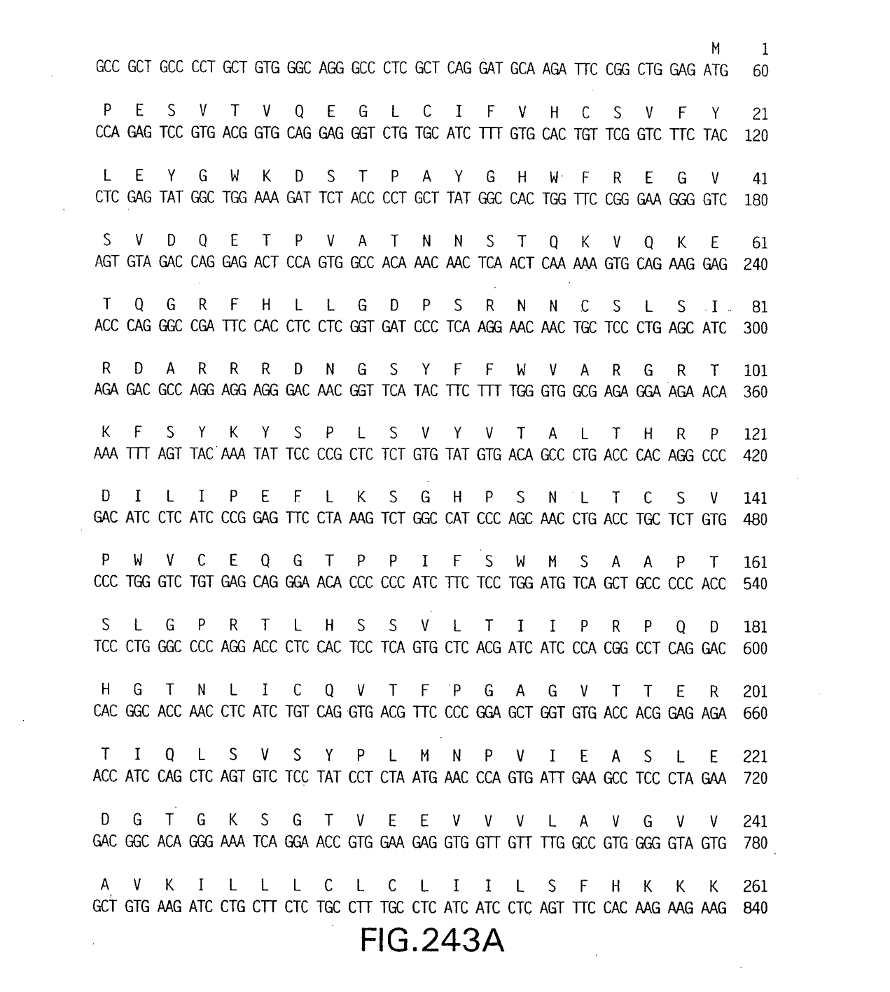

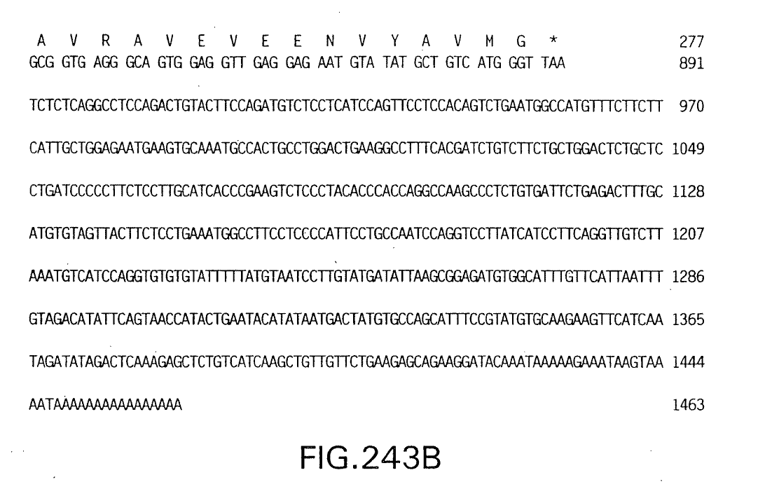

In another embodiment, TANGO 315 is referred to as TANGO 315 form 2. The open reading frame of TANGO 315 form 2, comprises nucleotides 58 to 888, and encodes a transmembrane protein comprising the amino acid sequence shown in FIG. 243A-243B (SEQ ID NO:153).

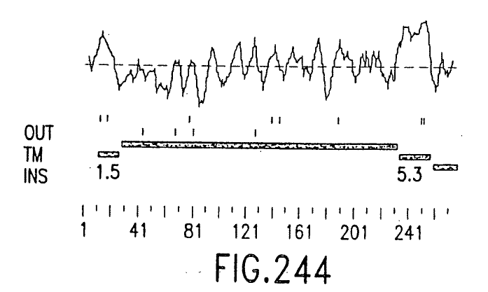

FIG. 244 depicts a hydropathy plot of the human TANGO 315 form 2 amino acid sequence depicted in FIG. 243A-243B.

The signal peptide of human TANGO 315 form 2 includes a 26 amino acid signal peptide (amino acid 1 to amino acid 26 of SEQ ID NO: 154) preceding the mature TANGO 315 form 2 protein (corresponding to amino acid 27 to amino acid 277 of SEQ ID NO: 154). The molecular weight of TANGO 315 form 2 protein without post-translational modifications is 30.6 kDa, and after cleavage of the signal peptide the molecular weight is 27.6 kDa.

Human TANGO 315 form 2 protein is a transmembrane protein comprising amino acids 1 to 277 (SEQ ID NO: 154). In particular, TANGO 315 form 2 contains an extracellular domain comprising amino acid residues 27 to 232, a transmembrane domain comprising amino acid residues 233 to 257 and a cytoplasmic domain comprising amino acid residues 258 to 277 of SEQ ID NO: 154.

Human TANGO 315 form 2 includes an Ig-like domain at amino acids 132 to 190 of SEQ ID NO:154.

Four N-glycosylation sites are present in TANGO 315 form 2. The first has the sequence NNST (at amino acid residues 52 to 55), the second has the sequence NCSL (at amino acid residues 76 to 79), the third has the sequence NGSY (at amino acid residues 89 to 92), and the fourth has the sequence NLTC (at amino acid residues 136 to 139). Six protein kinase C phosphorylation sites are present in TANGO 315 form 2. The first has the sequence TQK (at amino acid residues 55 to 57), the second has the sequence SIR (at amino acid residues 80 to 82), the third has the sequence SYK (at amino acid residues 104 to 106), the fourth has the sequence THR (at amino acid residues 118 to 120), the fifth has the sequence TER (at amino acid residues 199 to 201), and the sixth has the sequence TGK (at amino acid residues 224 to 226). TANGO 315 form 2 has four casein kinase II phosphorylation sites. The first has the sequence TVQE (at amino acid residues 6 to 9), the second has the sequence SIRD (at amino acid residues 80 to 83), the third has the sequence SLED (at amino acid residues 219 to 222), and the fourth has the sequence TVEE (at amino acid residues 229 to. 232). TANGO 315 form 2 has one tyrosine kinase phosphorylation site with the sequence RRRDNGSY at amino acid residues 85 to 92. Two N-myristylation sites are present in TANGO 315 form 2. The first has the sequence GAGVTT (at amino acid residues 194 to 199) and the second has the sequence GTGKSG (at amino acid residues 223 to 228).

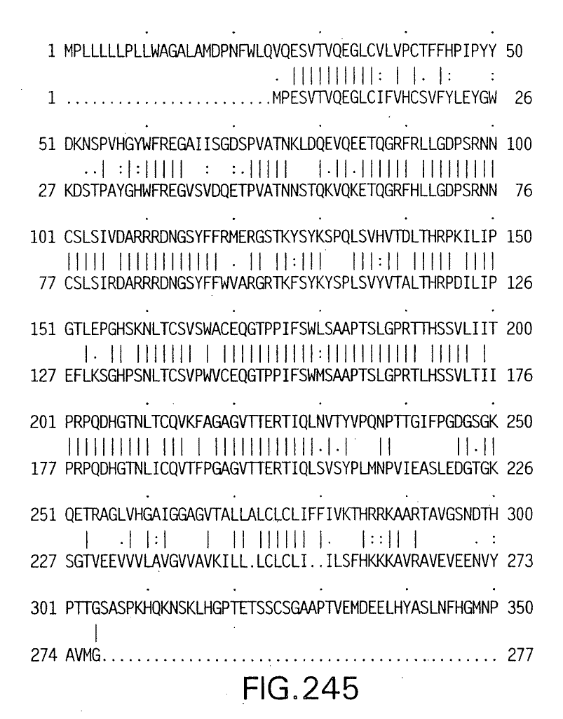

FIG. 245 depicts a local alignment of the amino acid of TANGO 315 form 2 and the amino acid sequence of CD33 (Accession Number NP_{—}001763). The alignment shows that there is a 62% overall amino acid sequence identity between TANGO 315 form 2 and CD33.

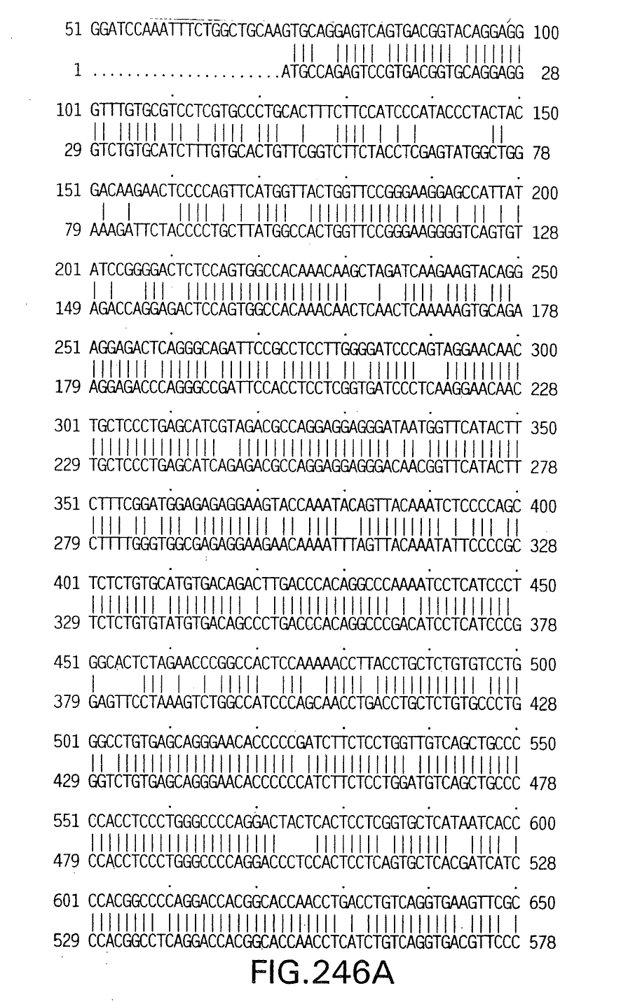

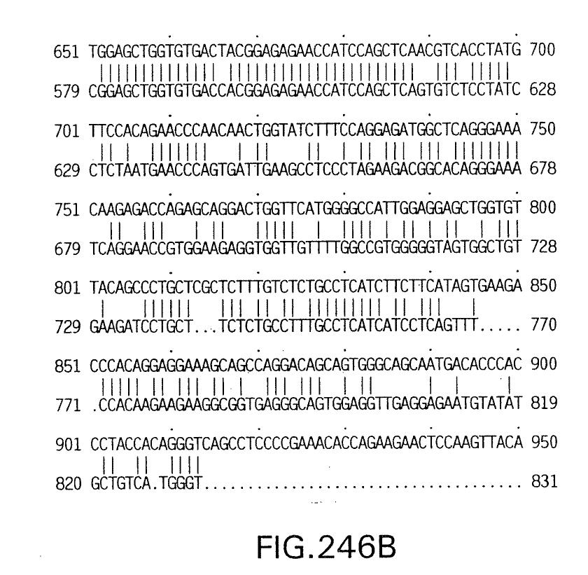

FIG. 246A-246B depicts a local alignment of the nucleotide sequence of CD33 (Accession Number NM_{—}001772) and the nucleotide sequence of human TANGO 315 form 2. The nucleotide sequences of the coding regions of CD33 and human TANGO 315 form 2 are 75.4% identical.

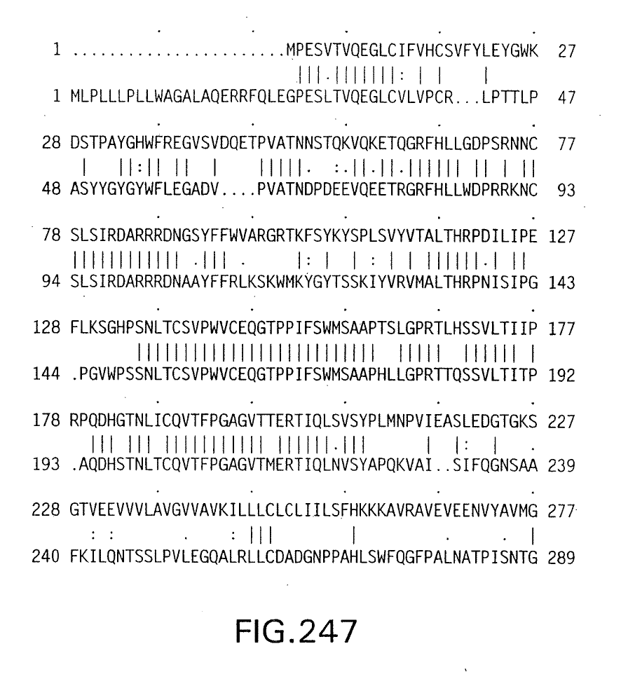

FIG. 247 depicts an alignment of the amino acid sequence of TANGO 315 form 2 and the amino acid sequence of OB-BP-1 (Accession Number AAB70702). The alignment shows that there is a 53.3% overall amino acid sequence identity between TANGO 315 form 2 and OB-BP-1.

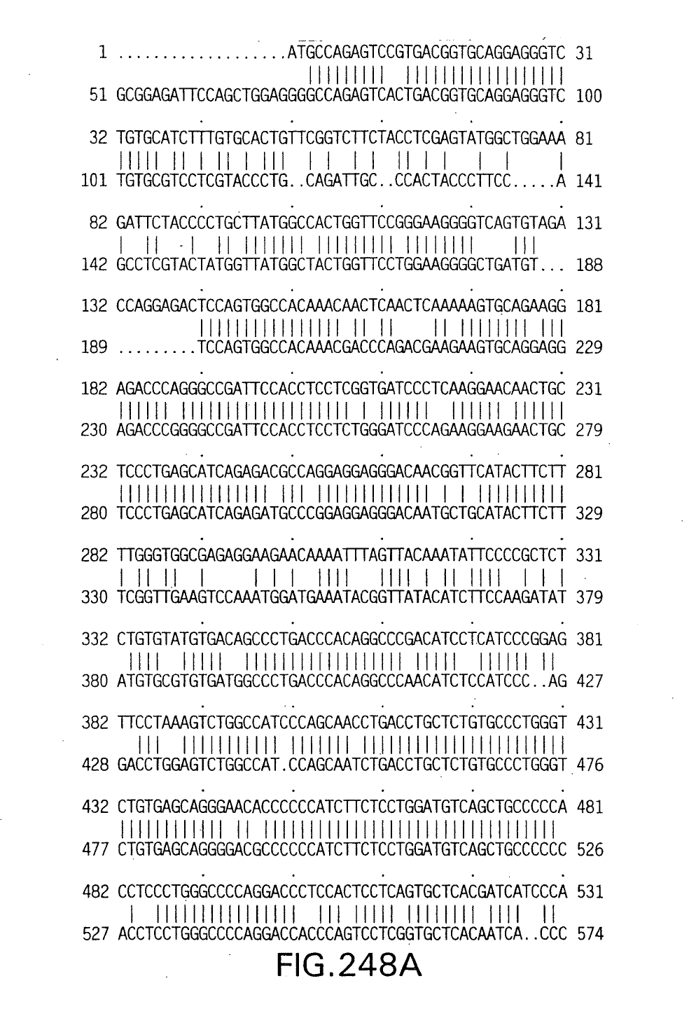

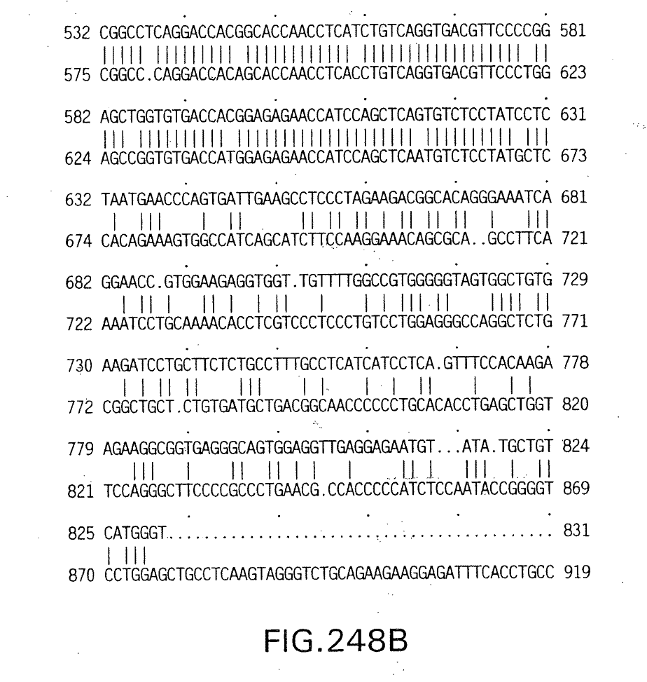

FIG. 248A-248B depicts an alignment of the nucleotide sequence of human TANGO 315 form 2 coding region and the nucleotide sequence of human OB-BP-1 coding region (Accession Number U71382). The nucleotide sequences of the coding regions are 73.2% identical.

TANGO 315 expression was detected in mast cell line (HMC-1 control) and d8 dendritic cells. No expression was detected in approximately 180 other tissues analyzed.

Clone EpT315, which encodes human TANGO 315, was deposited with the American Type Culture Collection (10801 University Boulevard, Manassas, Va. 20110-2209) on Oct. 1, 1999 and assigned PTA-816. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 315 Nucleic Acids, Polypeptides, and Modulators Thereof

As TANGO 315 was originally found in a human natural killer cell library, TANGO 315 nucleic acids, proteins, and modulators thereof can be used to modulate and/or track the proliferation, development, differentiation, maturation, activity and/or function of immune cells, e.g., natural killer cells, mast cells, and dendritic cells. TANGO 315 nucleic acids, proteins and modulators thereof can be utilized to modulate immune-related processes, e.g., the host immune response by, for example, modulating the formation of and/or binding to immune complexes, detection and defense against surface antigens and bacteria, and immune surveillance for rapid removal or pathogens. Such TANGO 315 nucleic acids, proteins and modulators thereof can be utilized to treat, e.g., to ameliorate incidence of any symptoms associated with disorders that involve such immune-related processes, including, but not limited to, viral or bacterial infection, and inflammatory disorders (e.g., bacterial or viral infection, psoriasis, allergies and inflammatory bowel diseases) and autoimmune disorders (e.g., transplant rejection and Hashimoto's disease). TANGO 315 nucleic acids, proteins and modulators thereof can be used to modulate, diagnose, monitor or treat immune related disorders, e.g., immunodeficiency disorders (e.g., HIV), viral disorders, cancers, and inflammatory disorders (e.g., bacterial or viral infection, psoriasis, septicemia, arthritis, allergic reactions). TANGO 315 nucleic acids, proteins and modulators thereof can be used to modulate, diagnose, monitor or treat atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy.

As TANGO 315 was cloned from a natural killer cell library, TANGO 315 nucleic acids, proteins and modulators thereof can also be used to diagnose, monitor and/or treat diseases associated with aberrant natural killer cell activation such as chronic natural killer cell lymphocytosis, aggressive non-T, non-B natural killer cell lymphoma/leukemia (ANKL/L), and Chediak-Higashi syndrome. Further, TANGO 315 nucleic acids, proteins and modulators thereof can be used to alleviate one or more symptoms associated with such disorders.

TANGO 315 is expressed by mast cells. Therefore, TANGO 315 nucleic acid, proteins and modulators thereof can also be utilized to diagnose, monitor modulate and/or treat disorders associated with aberrant mast cell proliferation, differentiation, maturation, activity and/or function. For example, TANGO 315 nucleic acids, proteins and modulators thereof can be utilized to treat inflammatory conditions (e.g. rhinitis, conjunctivitis, asthma and allergy) which involve or are mediated by mast activity.

TANGO 315 exhibits homology to CD33 (otherwise known as Siglec-3). CD33 is expressed by myelomonocytic cells and is a marker of disorders such as myeloid-related leukemia. Therefore, TANGO 315 nucleic acids, proteins and modulators thereof can be utilized to track and/or modulate the proliferation, differentiation, maturation, activity and/or function of myeloid cells. TANGO 315 nucleic acids, proteins and modulators thereof can be utilized to diagnose, monitor, modulate and/or treat disorders associated with abnormal function of myeloid cells. Such disorders can include, but are not limited to, myelodysplastic syndrome (MDS) acute myelogenous leukemia (AML), chronic myeloid leukemia, agnogenic myeloid (megakaryotic/granukaryotic metaplasia (AMM), and idiopathic myelofibrosis (IMF).

As TANGO 315 has homology to OB-BP-1, TANGO 315 nucleic acids, proteins and modulators thereof can be used to track and/or modulate adipocyte function and activity. TANGO 315 nucleic acids, proteins and modulators thereof can be used to track and/or modulate skeletal muscle function and activity. TANGO 315 nucleic acids, proteins and modulators thereof can be used to track and/or modulate neuroendocrine function, e.g., neuroendocrine secretion (e.g., secretion of growth hormone, melatonin, opioids, corticotropin-releasing hormones and cytokines). TANGO 315 nucleic acids, proteins and modulators thereof can be used to diagnose, monitor, modulate and/or treat (that is, alleviate a symptom of) obesity, anorexia nervosa, diabetes mellitus, polycystic ovary syndrome, acquired immunodeficiency syndrome, cancer, nephropathy, thyroid disease, Cushing's syndrome, and growth hormone deficiency.

In further light of TANGO 315's homology to OB-BP-1, TANGO 315 nucleic acids, proteins, and modulators thereof can be used to track and/or modulate embryonic development. TANGO 315 nucleic acids, proteins and modulators thereof can be used to diagnose, monitor, modulate and/or treat embryonic disorders.

TANGO 315 nucleic acids, proteins and modulators thereof can be used to track and/or modulate intracellular signaling. TANGO 315 nucleic acids, proteins and modulators thereof can also be utilized to modulate immune activation, for example, antagonists to TANGO 315 action, such as peptides, antibodies or small molecules that decrease or block TANGO 315 activity, e.g., binding to extracellular matrix components, e.g., integrins, or that prevent TANGO 315 signaling, can be used as immune system activation blockers. In another example, agonists that mimic or partially mimic TANGO 315 activity, such as peptides, antibodies or small molecules, can be used to induce immune system activation. Antibodies may activate or inhibit the cell adhesion, proliferation and activation, and may help in treating infection, autoimmunity, inflammation, and cancer by affecting these cellular processes. Further, TANGO 315 nucleic acids, proteins and modulators thereof can be utilized to track and/or modulate intercellular signaling in the immune system. For example, TANGO 315 nucleic acids, proteins and modulators thereof can be used to modulate intercellular signal transduction in immune stimulation or suppression and modulate immune cell membrane adhesion to ECM components, during development, e.g., late stages of development.

TANGO 315 nucleic acids and/or proteins can be utilized as markers for immune cells (e.g., T cells, B cells, natural killer cells, and mast cells) and/or adipocytes. Further, TANGO 315 nucleic acids can be utilized for chromosomal mapping, or as chromosomal markers, e.g., in radiation hybrid mapping.

### Human TANGO 330 Form 1

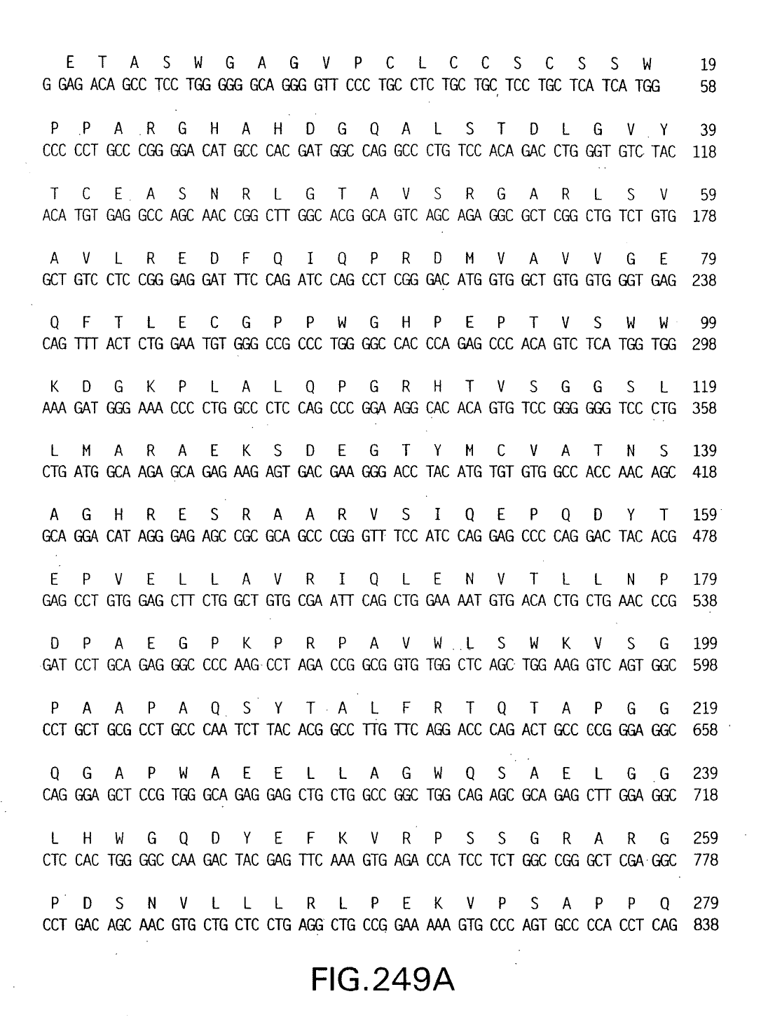

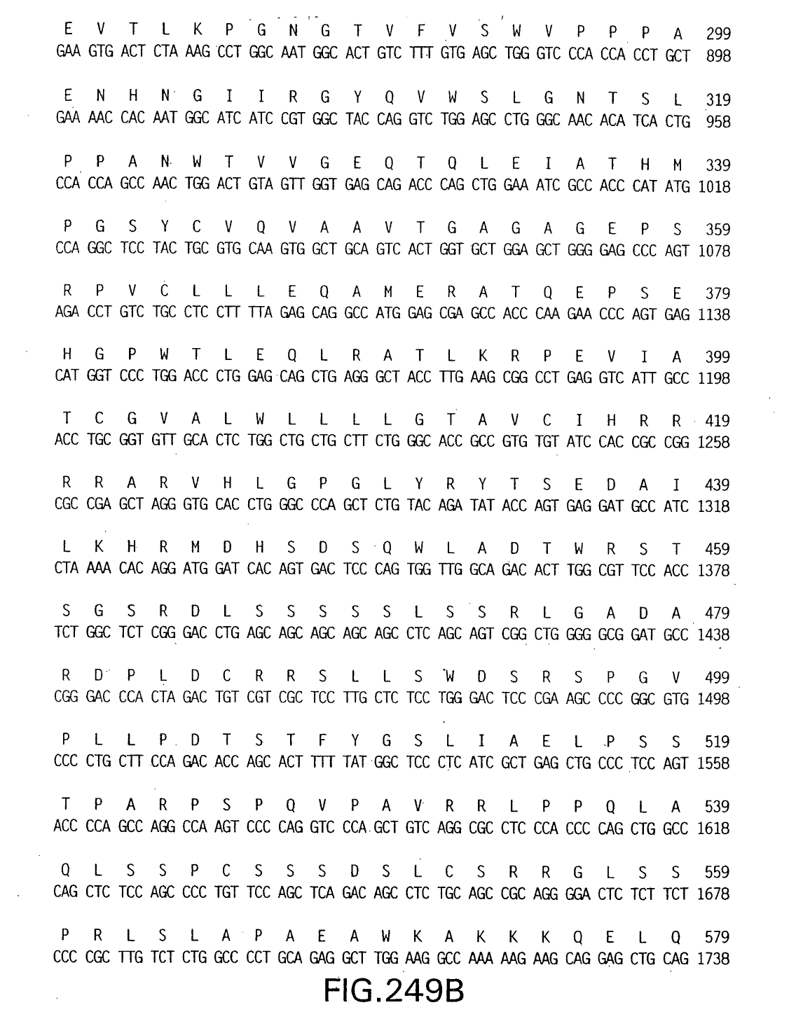

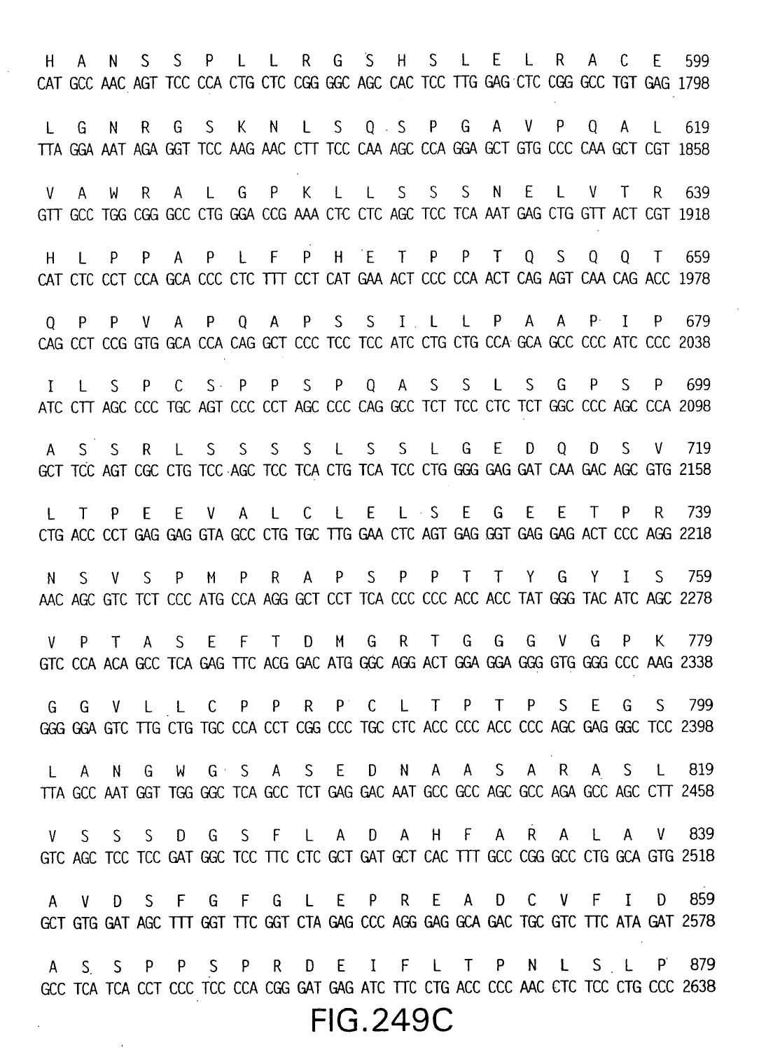

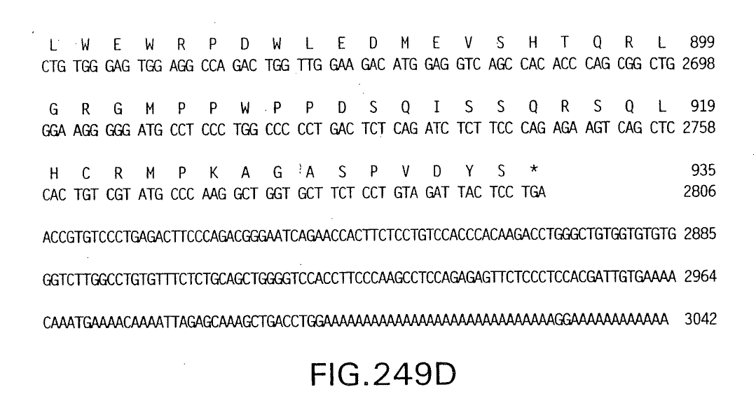

A cDNA encoding human TANGO 330 was identified by analyzing the sequences of clones present in an adrenal gland library for sequences that encode wholly secreted or transmembrane proteins. This analysis led to the identification of a clone, jthAa060g22 encoding human TANGO 330 form 1. The human TANGO 330 form 1 cDNA of this clone comprises 3042 nucleotides (FIG. 249A-249D; SEQ ID NO: 155). The open reading frame of this cDNA, nucleotides 2 to 2803, encodes a transmembrane protein comprising the 934 amino acid sequence depicted in SEQ ID NO: 156. The molecular weight of the TANGO 330 form 1 protein without post-translational modifications is 99.9 kDa.

Human TANGO 330 form 1 protein is a transmembrane protein comprising amino acids 1 to 934. In particular, TANGO 330 form 1 contains an extracellular domain comprising amino acid residues 1 to 393, a transmembrane domain comprising acid residues 394 to 417, and cytoplasmic domains comprising amino acid residues 418 to 934 of SEQ ID NO:156.

Alternatively, in another embodiment, a human TANGO 330 form 1 protein is a transmembrane protein that contains a cytoplasmic domain comprising amino acid residues 1 to 393, a transmembrane domain comprising acid residues 394 to 417, and an extracellular domains comprising amino acid residues 418 to 934 of SEQ ID NO:156.

In one embodiment a cDNA sequence of human TANGO 330 form 1 has a nucleotide at position 3 which is guanine (G). In this embodiment, the cDNA contains an open reading frame encoding a polypeptide having an amino acid at position 1 that is glutamate (E). In an alternative embodiment, a species variant cDNA sequence of human TANGO 330 form 1 has a nucleotide at position 3 which is cytosine (C). In this embodiment, the cDNA contains an open reading frame encoding a polypeptide having an amino acid at position 1 that is aspartate (D), i.e., a conservative substitution.

In another embodiment a cDNA sequence of human TANGO 330 form 1 has a nucleotide at position 4 which is adenine (A). In this embodiment, the cDNA contains an open reading frame encoding a polypeptide having an amino acid at position 2 that is threonine (T). In an alternative embodiment, a species variant cDNA sequence of human TANGO 330 form 1 has a nucleotide at position 4 which is thymidine (T). In this embodiment, the cDNA contains an open reading frame encoding a polypeptide having an amino acid at position 2 that is serine (S), i.e., a conservative substitution.

In another embodiment a cDNA sequence of human TANGO 330 form 1 has a nucleotide at position 8 which is cytosine (C). In this embodiment, the cDNA contains an open reading frame encoding a polypeptide having an amino acid at position 3 that is alanine (A). In an alternative embodiment, a species variant cDNA sequence of human TANGO 330 form 1 has a nucleotide at position 8 which is thymidine (T). In this embodiment, the cDNA contains an open reading frame encoding a polypeptide having an amino acid at position 3 that is valine (V), i.e., a conservative substitution.

In another embodiment a cDNA sequence of human TANGO 330 form 1 has a nucleotide at position 158 which is guanine (G). In this embodiment, the cDNA contains an open reading frame encoding a polypeptide having an amino acid at position 53 that is arginine (R). In an alternative embodiment, a species variant cDNA sequence of human TANGO 330 form 1 has a nucleotide at position 158 which is adenine (A). In this embodiment, the cDNA contains an open reading frame encoding a polypeptide having an amino acid at position 53 that is lysine (K), i.e., a conservative substitution.

Human TANGO 330 form 1 has six N-glycosylation sites with the first sequence NVTL (at amino acid residues 173 to 176), the second has the sequence NGTV (at amino acid residues 287 to 290), the third has the sequence NTSL (at amino acid residues 316 to 319), the fourth has the sequence NWTV (at amino acid residues 323 to 326), the fifth has the sequence NLSQ (at amino acid residues 607 to 610), and the sixth has the sequence NLSL (at amino acid residues 875 to 878).

Fifteen protein kinase C phosphorylation sites are present in TANGO 330. The first has the sequence SNR (at amino acid residues 44 to 46), the second has the sequence SWK (at amino acid residues 194 to 196), the third has the sequence SGR (at amino acid residues 254 to 256), the fourth has the sequence TLK (at amino acid residues 282 to 284), the fifth has the sequence TLK (at amino acid residues 391 to 393), the sixth has the sequence TWR (at amino acid residues 455 to 457), the seventh has the sequence SSR (at amino acid residues 472 to 474), the eighth has the sequence SRR (at amino acid residues 553 to 555), the ninth has the sequence SPR (at amino acid residues 559 to 561), the tenth has the sequence SSR (at amino acid residues 701 to 703), the eleventh has the sequence TPR (at amino acid residues 737 to 739), the twelfth has the sequence SAR (at amino acid residues 814 to 816), the seventh has the sequence SPR (at amino acid residues 865 to 867), the fourteenth has the sequence TQR (at amino acid residues 896 to 898), and the fifteenth has the sequence SQR (at amino acid residues 914 to 916).

Human TANGO 330 has fourteen casein kinase II phosphorylation sites. The first has the sequence SIQE (at amino acid residues 151 to 154), the second has the sequence TQLE (at amino acid residues 331 to 334), the third has the sequence TSED (at amino acid residues 434 to 437), the fourth has the sequence SSSD (at amino acid residues 546 to 559), the fifth has the sequence SSNE (at amino acid residues 632 to 635), the sixth has the sequence SLGE (at amino acid residues 711 to 714), the seventh has the sequence TPEE (at amino acid residues 721 to 724), the eighth has the sequence SEGE (at amino acid residues 5732 to 735), the ninth has the sequence TASE (at amino acid residues 762 to 765), the tenth has the sequence TPSE (at amino acid residues 794 to 797), the eleventh has the sequence SASE (at amino acid residues 806 to 809), the twelfth has the sequence SSSD (at amino acid residues 821 to 824), the thirteenth has the sequence SPRD (at amino acid residues 865 to 868), and the fourteenth has the sequence SPVD (at amino acid residues 929 to 932).

Human TANGO 330 has a tyrosine kinase phosphorylation site with the sequence KSDEGTY (at amino acid residues 126 to 132).

Human TANGO 330 has fourteen N-myristoylation sites. The first has the sequence GQALST (at amino acid residues 29 to 34), the second has the sequence GVYTCE (at amino acid residues 37 to 42), the third has the sequence GTAVSR (at amino acid residues 48 to 53), the fourth has the sequence GARLSV (at amino acid residues 54 to 59), the fifth has the sequence GTYMCV (at amino acid residues 130 to 135), the sixth has the sequence GAPWAE (at amino acid residues 221 to 226), the seventh has the sequence GLHWGQ (at amino acid residues 239 to 244), the eighth has the sequence GIIRGY (at amino acid residues 304 to 309), the ninth has the sequence GAGAGE (at amino acid residues 352 to 357) the tenth has the sequence GTAVCI (at amino acid residues 411 to 416), the eleventh has the sequence GSLIAE (at amino acid residues 510 to 515), the twelfth has the sequence GNRGSK (at amino acid residues 601 to 606), the thirteenth has the sequence GSLANG (at amino acid residues 798 to 803), and the fourteenth has the sequence GSFLAD (at amino acid residues 825 to 830).

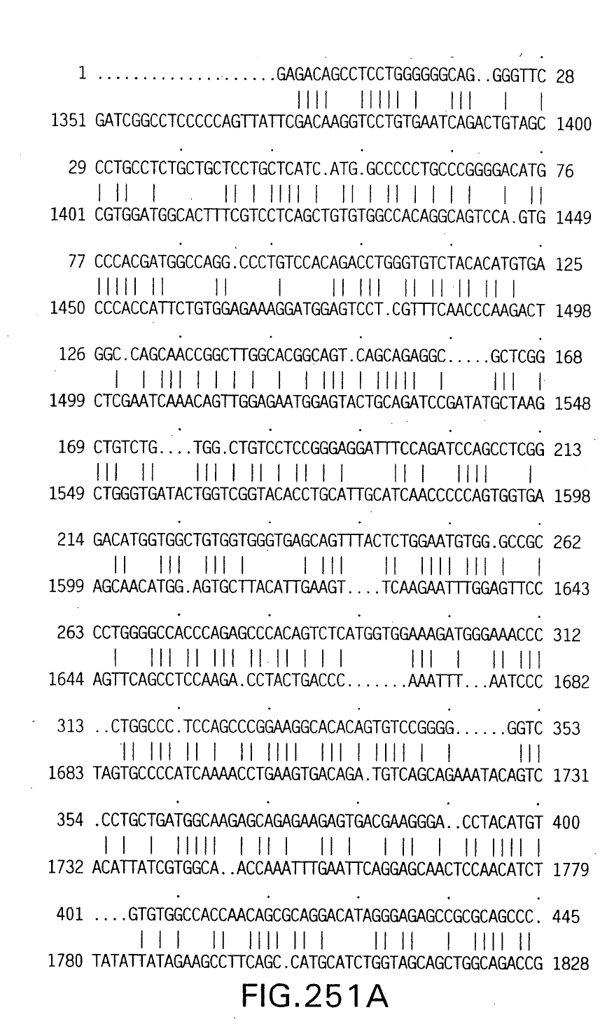

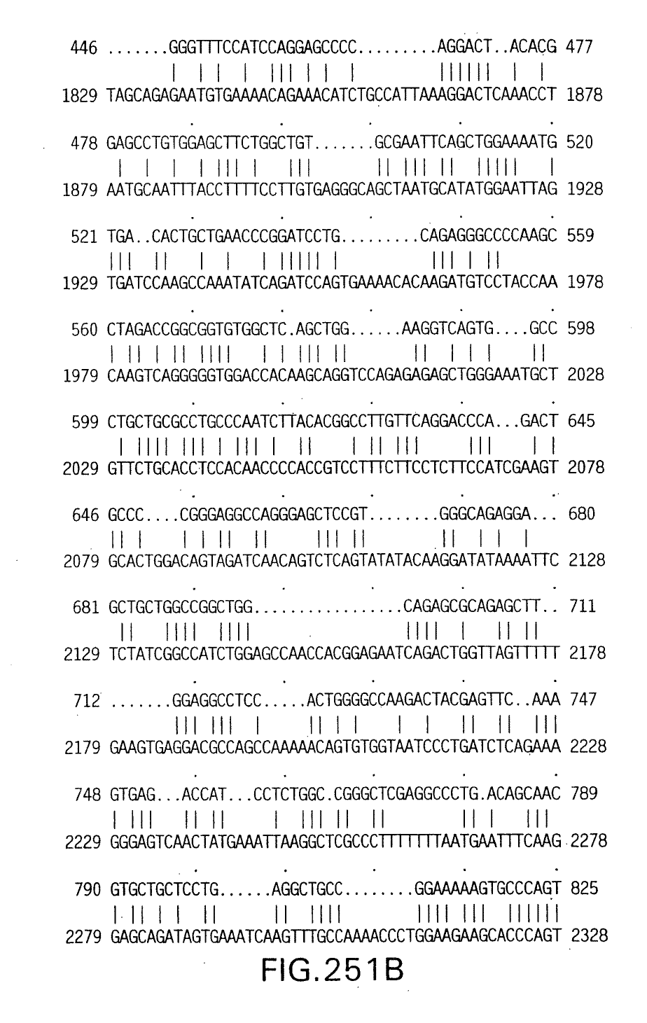

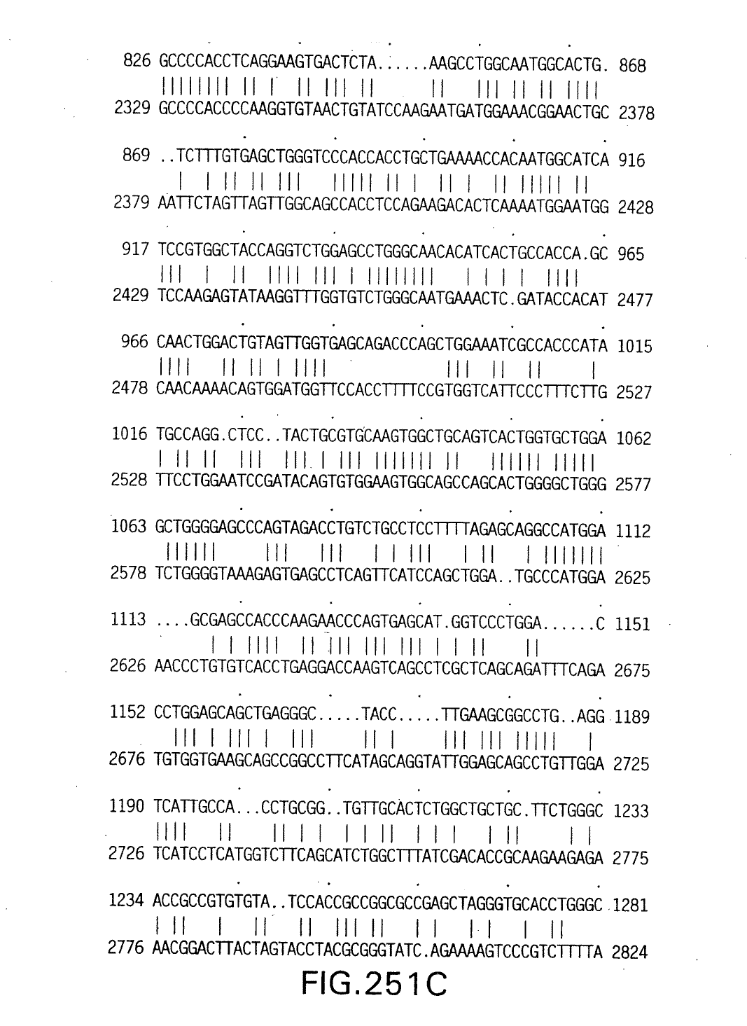

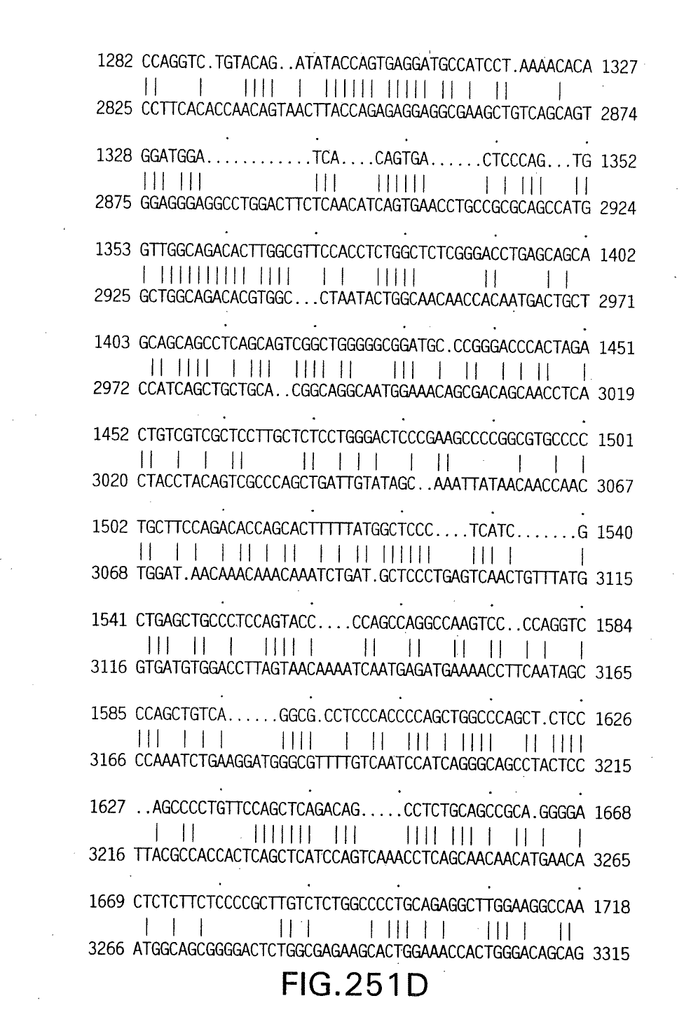

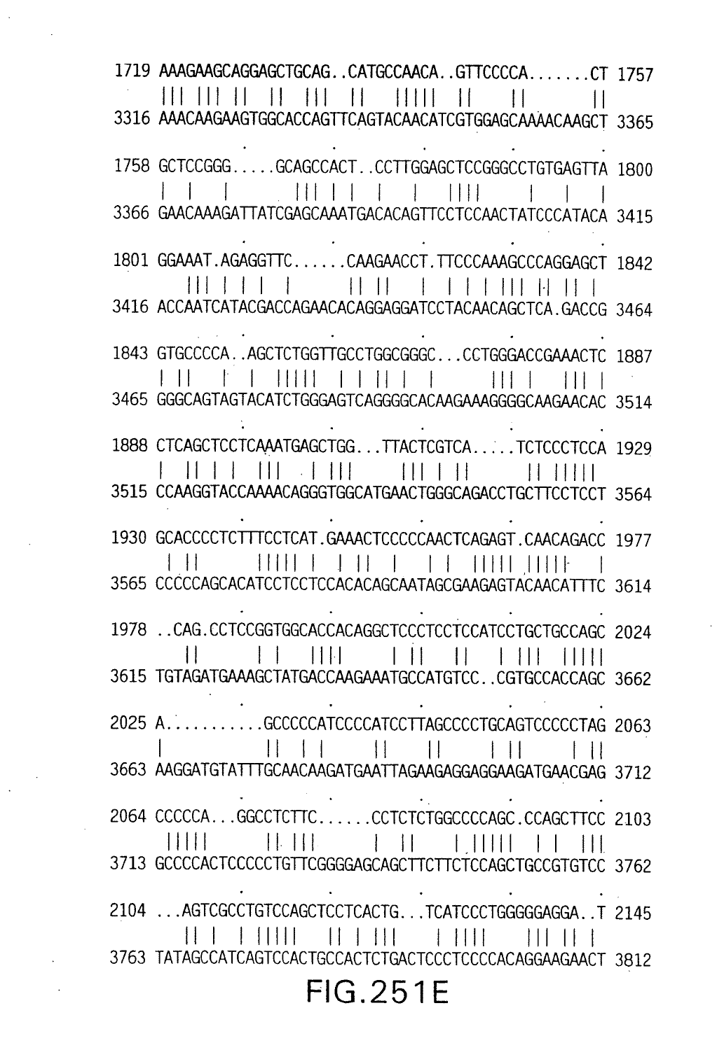

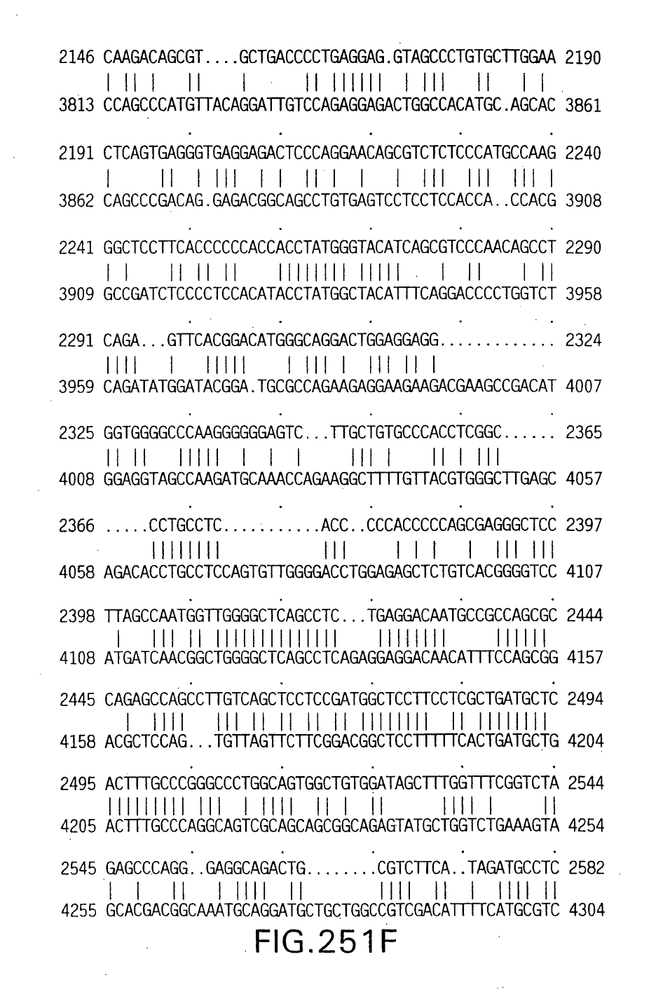

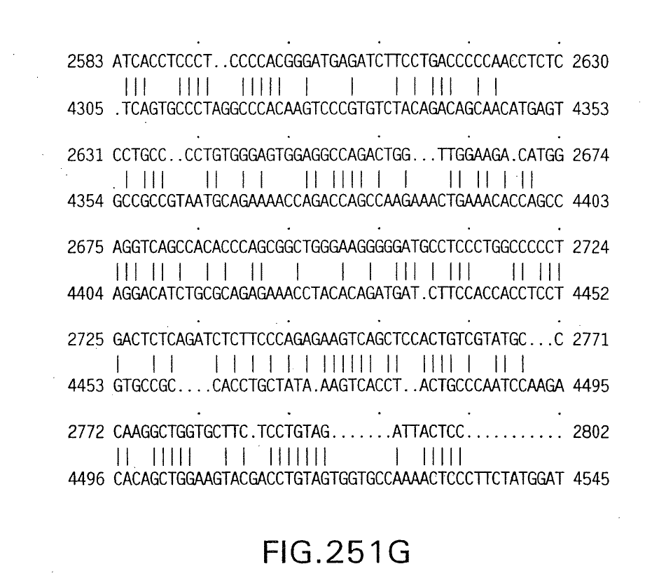

FIG. 251A-251G depicts a local alignment of the nucleotide sequence of human Roundabout (Accession Number AF040990) and the nucleotide sequence of human TANGO 330 form 1 shown in. The aligned nucleotide sequences of human Roundabout and human TANGO 330 form 1 are 56.9% identical. This alignment was performed using the ALIGN alignment program with a PAM120 scoring matrix, a gap length penalty of 12, and a gap penalty of 4.

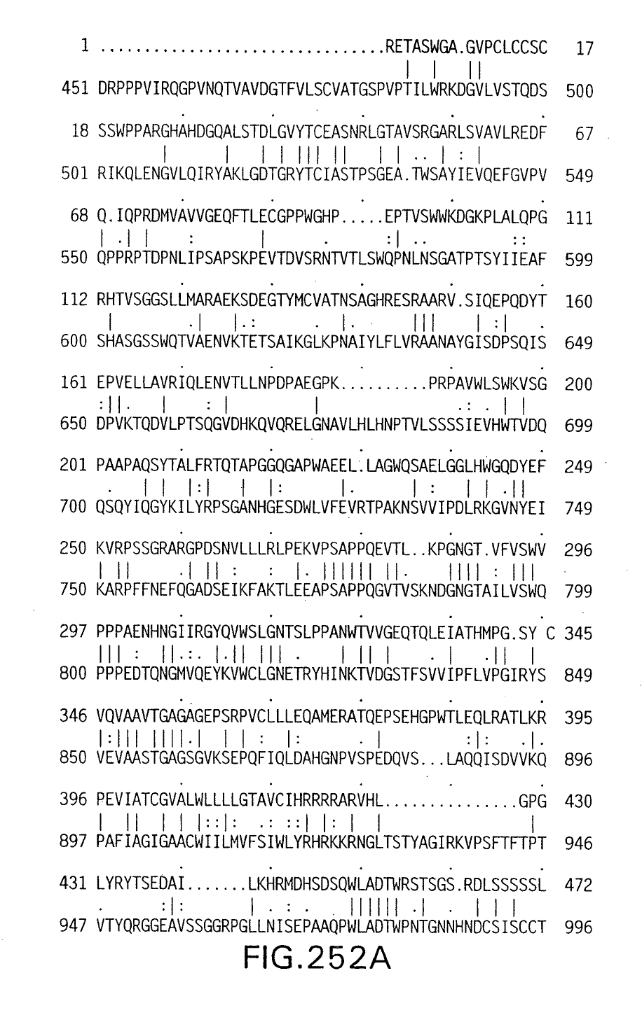

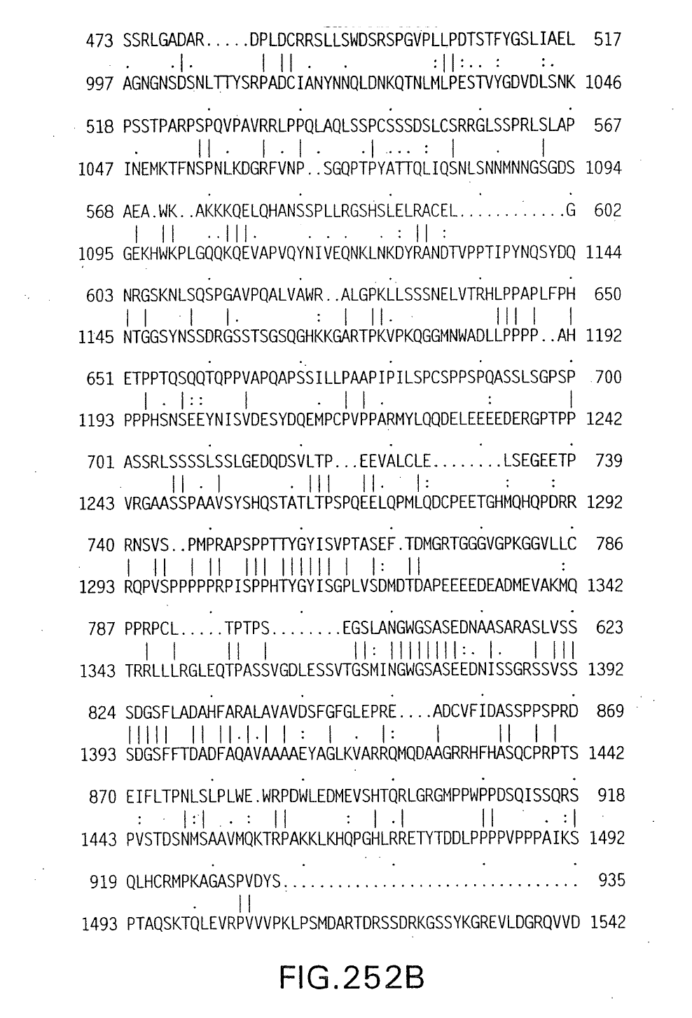

FIG. 252A-252B depicts an alignment of the amino acid sequence of human Roundabout (Accession Number AAC39575) and the amino acid sequence of human TANGO 330 depicted in. The amino acid sequences of human Roundabout and human TANGO 330 are 26.6% identical. This alignment was performed using the ALIGN alignment program with a PAM120 scoring matrix, a gap length penalty of 12, and a gap penalty of 4.

Clone 330a, which encodes human TANGO 330, was deposited with the American Type Culture Collection (10801 University Boulevard, Manassas, Va. 20110-2209) on Oct. 1, 1999 and assigned PTA-816. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Human TANGO 330 Form 2

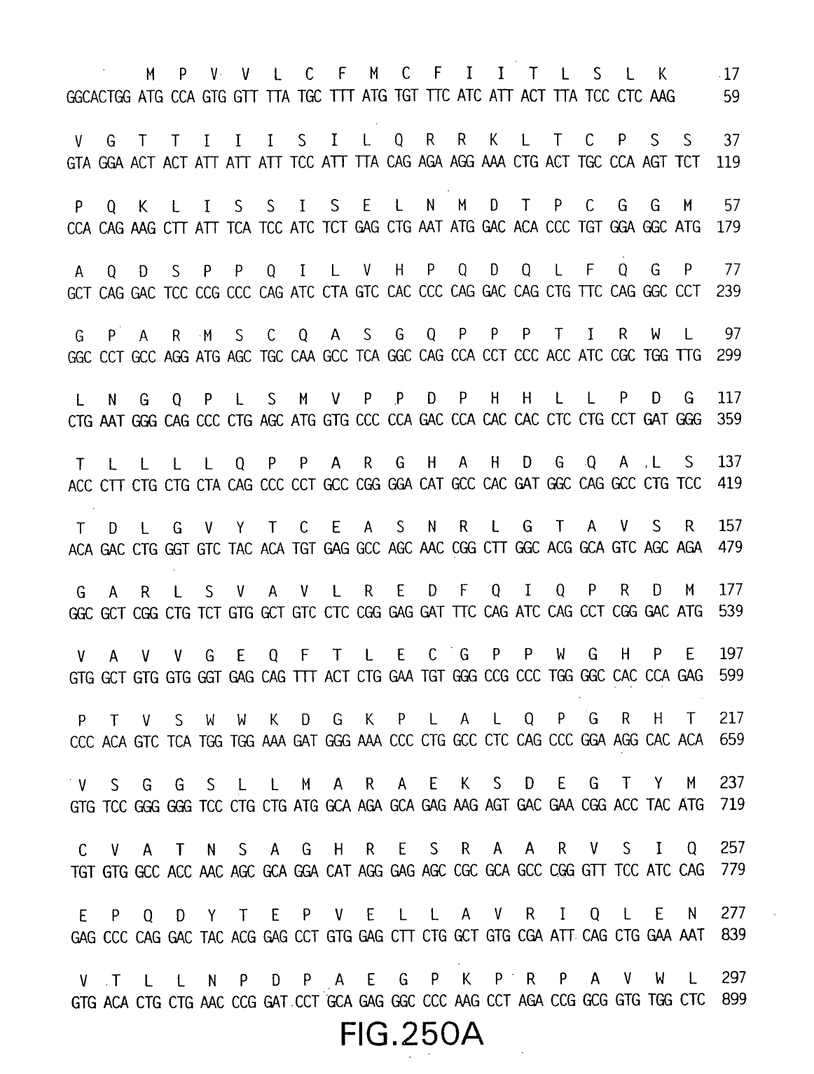

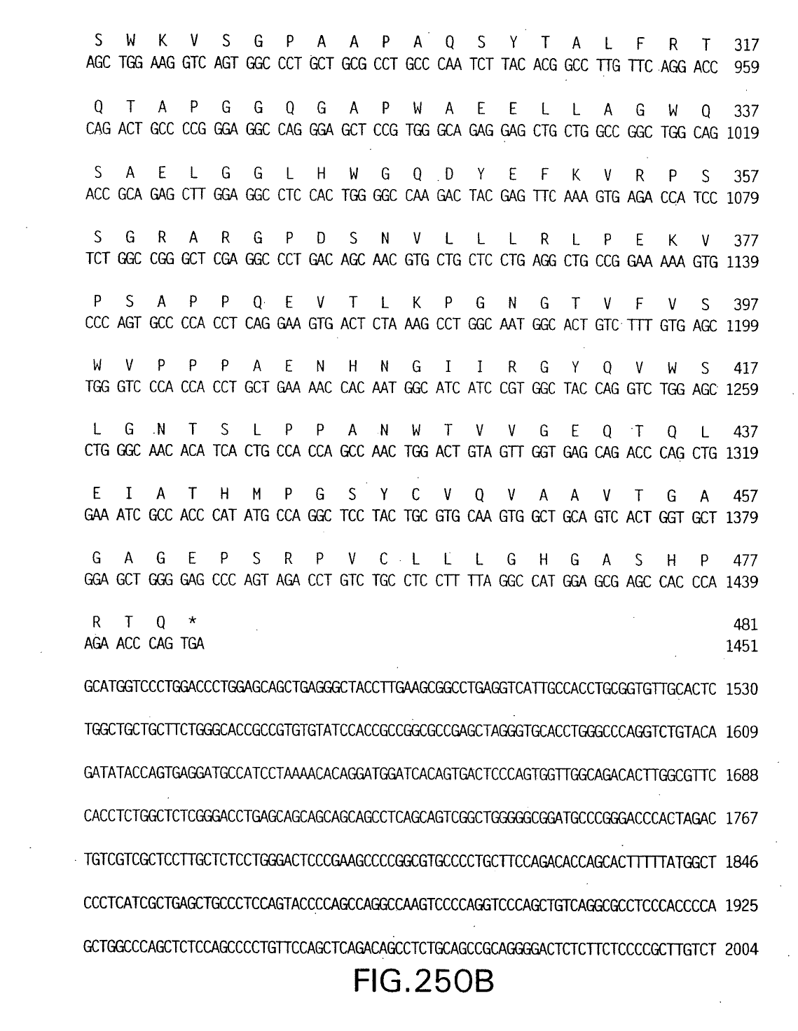

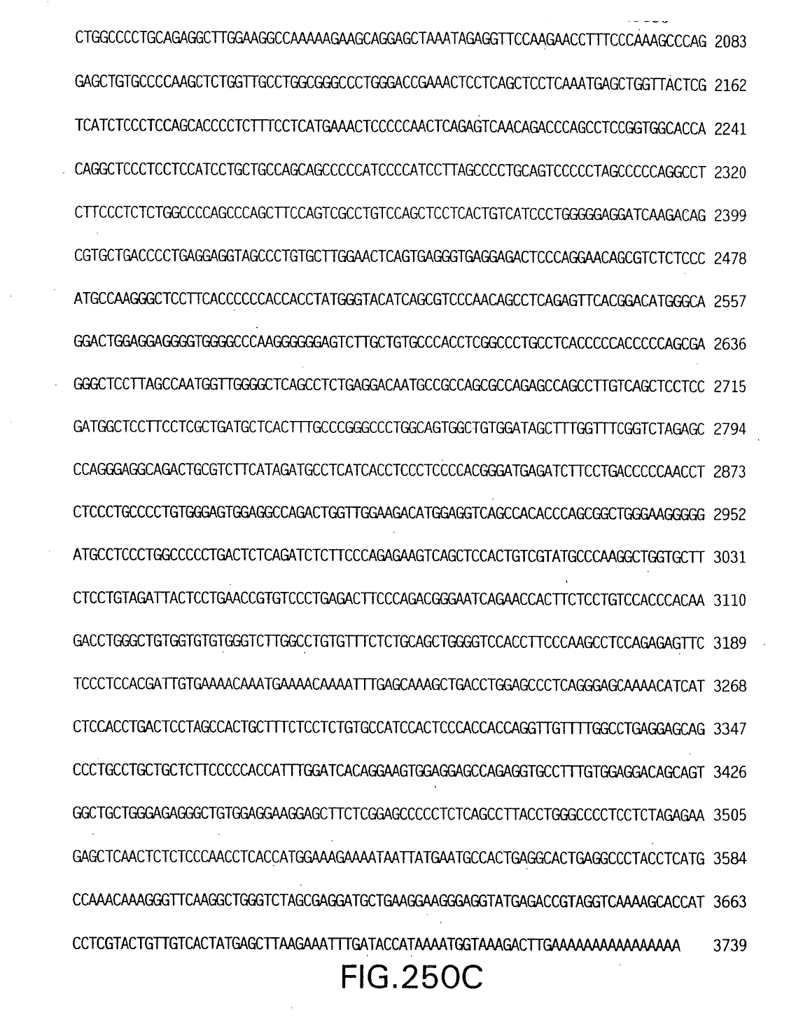

A cDNA encoding human TANGO 330 was identified by analyzing the sequences of clones present in an astrocyte library for sequences that encode wholly secreted or transmembrane proteins. This analysis led to the identification of a clone, Jthxe181e12, encoding human TANGO 330 form 2. The human TANGO 330 form 2 cDNA of this clone comprises 3808 nucleotides (FIG. 250A-250C; SEQ ID NO:157). The open reading frame of this cDNA, nucleotides 9 to 1448, encodes a secreted protein comprising the 480 amino acid sequence depicted in SEQ ID NO: 158.

The signal peptide prediction program SIGNALP (Nielsen et al., 1997, Protein Engineering 10:1-6) predicted that human TANGO 330 form 2 includes a 20 amino acid signal peptide (amino acid 1 to amino acid 20 of SEQ ID NO:158) preceding the mature TANGO 330 form 2 protein (corresponding to amino acid 21 to amino acid 480 of SEQ ID NO: 158). The molecular weight of a TANGO 330 protein without post-translational modification is 51.5 kda, and after cleavage of the signal peptide the molecular weight of TANGO 330 form 2 is 49.3 kDa.

In instances wherein the signal peptide is not cleaved, a human TANGO 330 form 2 protein is a transmembrane protein that contains an extracellular domain corresponding to amino acids 21 to 480 and a transmembrane domain amino acids 1 to 20 of SEQ ID NO:158.

Human TANGO 330 form 2 has four N-glycosylation sites with the first sequence NVTL (at amino acid residues 277 to 280), the second has the sequence NGTV (at amino acid residues 391 to 394), the third has the sequence NTSL (at amino acid residues 420 to 423), and the fourth has the sequence NWTV (at amino acid residues 427 to 430).

Human TANGO 330 form 2 has one cAMP and cGMP dependent protein kinase phosphorylation site which has the sequence RKLT (at amino acid residues 30 to 33).

Six protein kinase C phosphorylation sites are present in TANGO 330 form 2. The first has the sequence SLK (at amino acid residues 15 to 17), the second has the sequence TIR (at amino acid residues 93 to 95), the third has the sequence SNR (at amino acid residues 148 to 150), the fourth has the sequence SWK (at amino acid residues 298 to 300), the fifth has the sequence SGR (at amino acid residues 358 to 360), and the sixth has the sequence TLK (at amino acid residues 386 to 388).

Human TANGO 330 has three casein kinase II phosphorylation sites. The first has the sequence SISE (at amino acid residues 44 to 47), the second has the sequence SIQE (at amino acid residues 255 to 258), the third has the sequence TQLE (at amino acid residues 435 to 438).

Human TANGO 330 has a tyrosine kinase phosphorylation site with the sequence KSDEGTY (at amino acid residues 230 to 236).

Human TANGO 330 has ten N-myristoylation sites. The first has the sequence GQPLSM (at amino acid residues 100 to 105), the second has the sequence GQALST (at amino acid residues 133 to 138), the third has the sequence GVYTCE (at amino acid residues 141 to 146), the fourth has the sequence GTAVSR (at amino acid residues 152 to 157), the fifth has the sequence GARLSV (at amino acid residues 158 to 163), the sixth has the sequence GTYMCV (at amino acid residues 234 to 239), the seventh has the sequence GAPWAE (at amino acid residues 325 to 330), the eighth has the sequence GLHWGQ (at amino acid residues 343 to 348), the ninth has the sequence GIIRGY (at amino acid residues 408 to 413), and the tenth has the sequence GAGAGE (at amino acid residues 456 to 461).

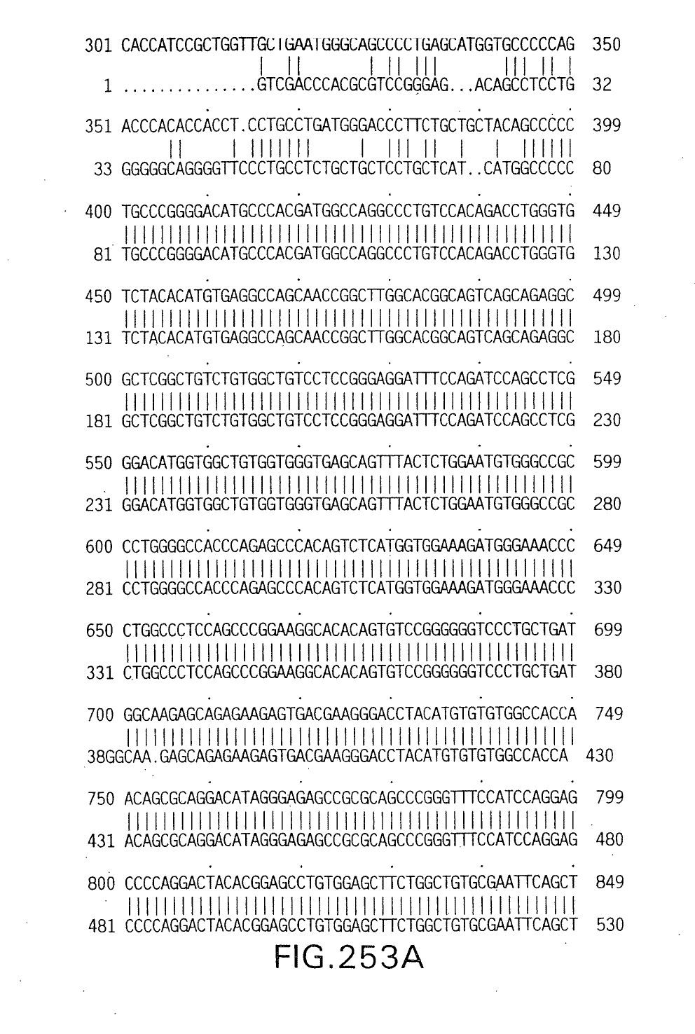

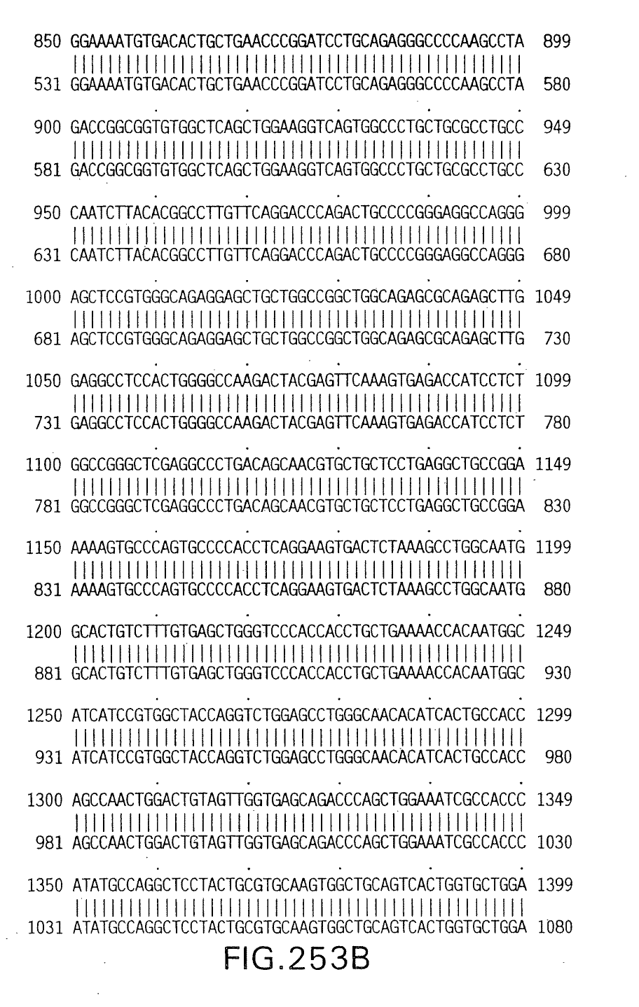

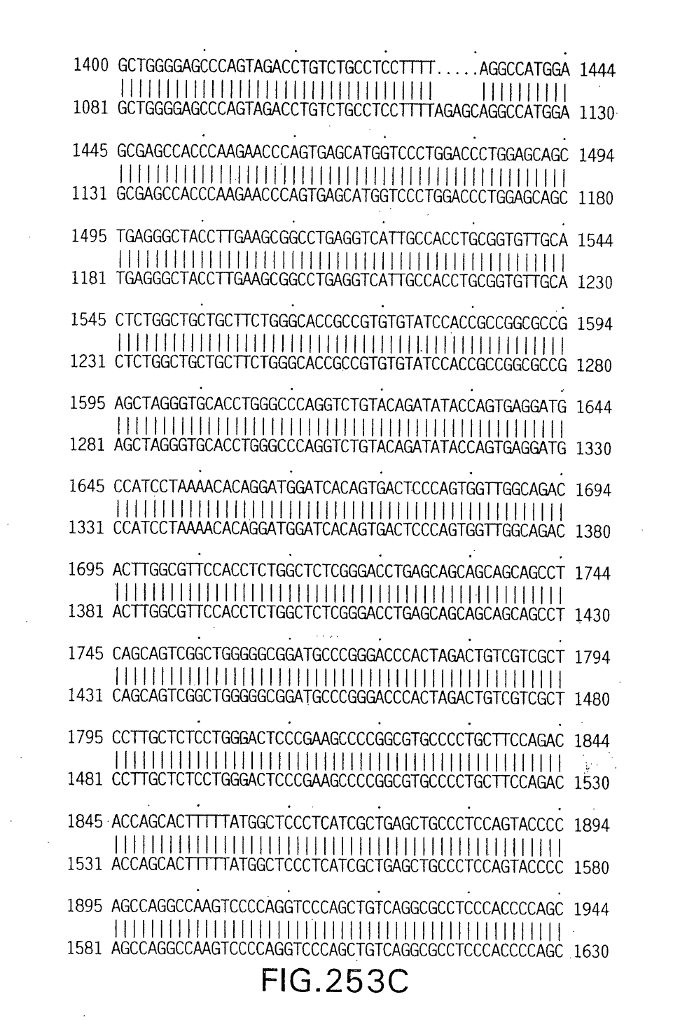

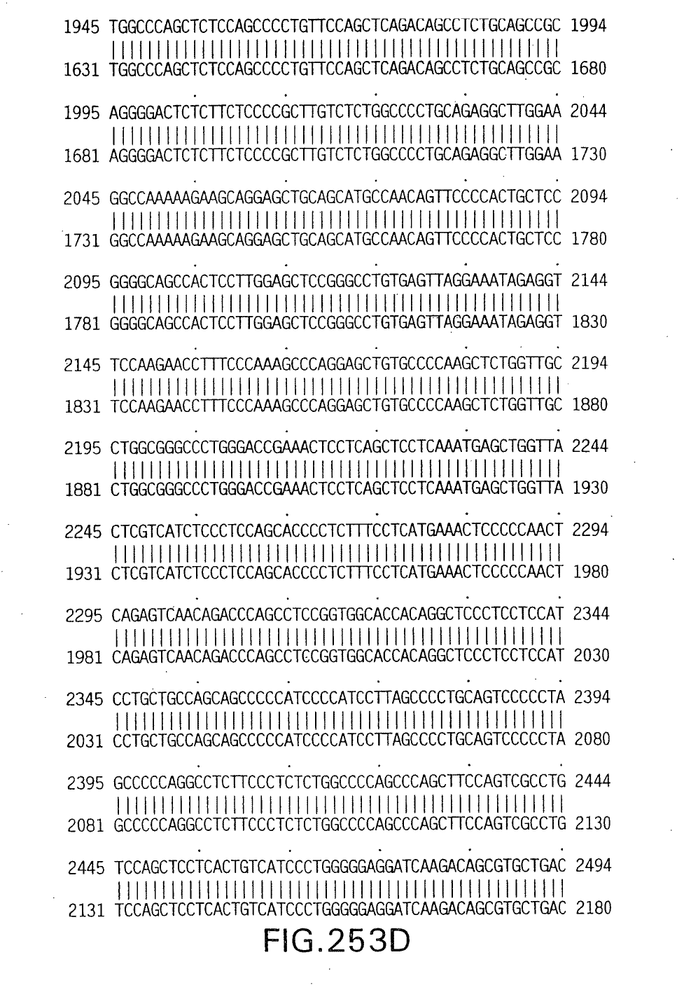

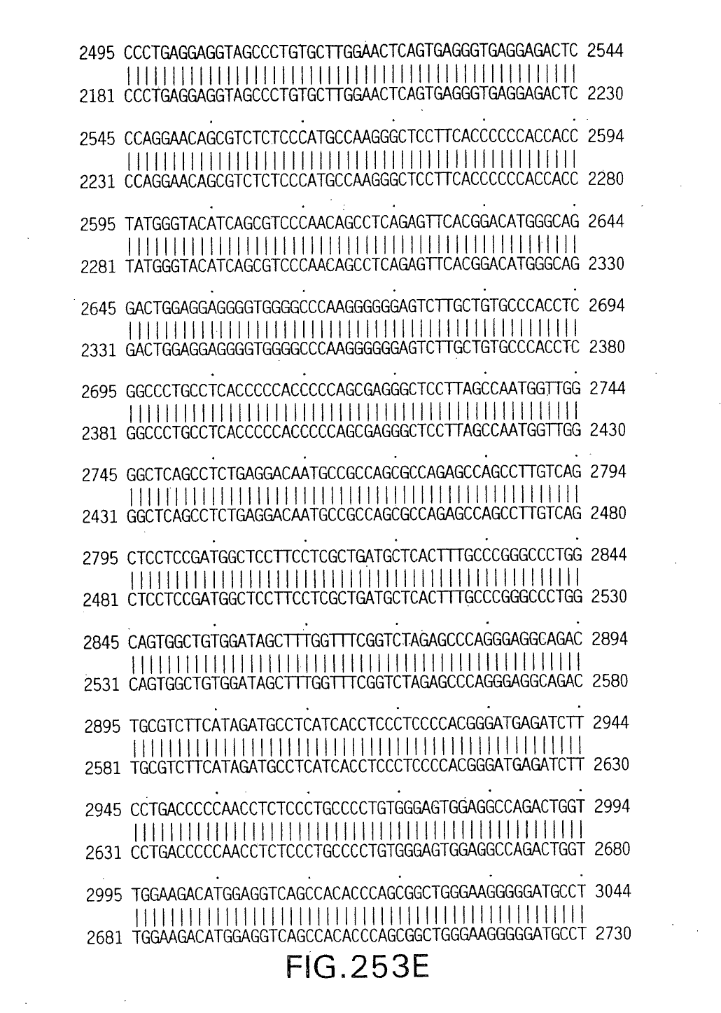

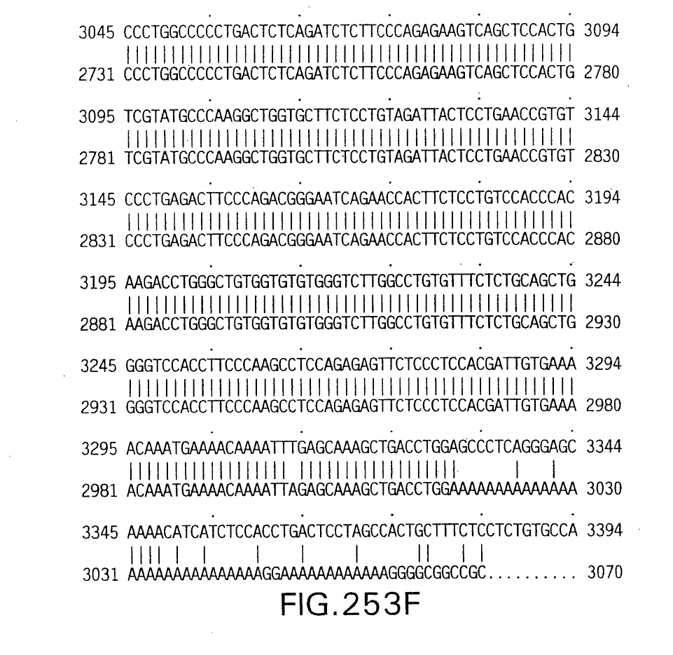

FIG. 253A-253F depicts an alignment of the nucleotide sequence of TANGO 330 form 1 and the nucleotide sequence of human TANGO 330 form 2. The nucleotide sequences of TANGO 330 form 1 and TANGO 330 form 2 are 97.4% identical over the local area of similar nucleotides. TANGO 330 form 1 and form 2 differ 5′ of nucleotide 394 of TANGO 330 form 2 and 5′ of nucleotide 75 of TANGO 330 form 2. In addition, TANGO 330 form 2 has a five base pair deletion at nucleotide 1336, corresponding to nucleotides 1116 to 1120 of TANGO 330 form 1 resulting in a frameshift that leads to a truncation of the protein immediately prior to the nucleotides that encode for the transmembrane domain. These alignments were performed using the ALIGN alignment program with a PAM120 scoring matrix, a gap length penalty of 12, and a gap penalty of 4.

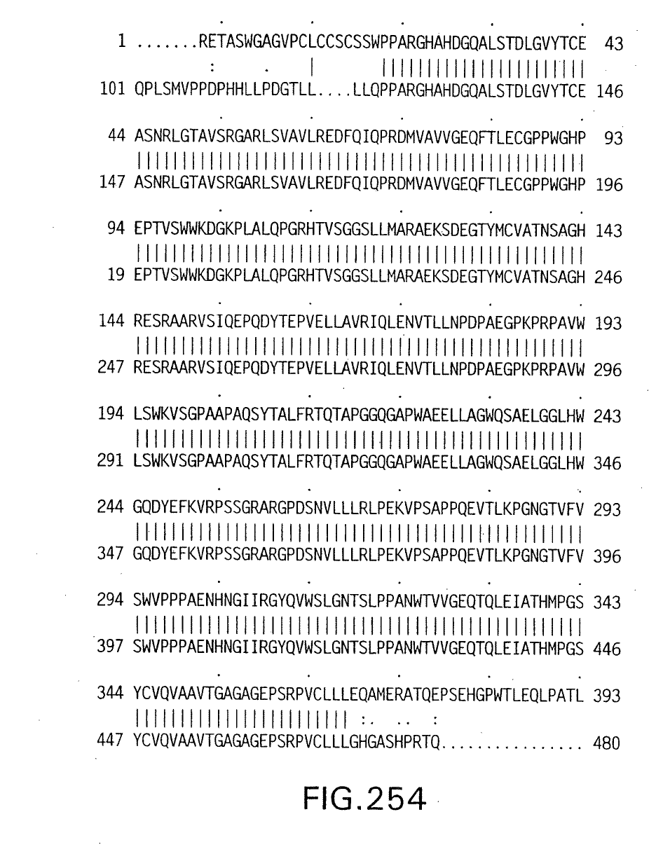

FIG. 254 depicts an alignment of the amino acid sequence of TANGO 330 form 1 shown in and the amino acid sequence of TANGO 330 form 2 shown in. The amino acid sequences of TANGO 330 form 1 and TANGO 330 form 2, are 94.1% identical over the 480 contiguous amino acids of TANGO 330 form 2 and the portion of the corresponding amino acid sequence of TANGO 330 form 1. This alignment was performed using the ALIGN alignment program with a PAM120 scoring matrix, a gap length penalty of 12, and a gap penalty of 4.

Clone 330b, which encodes human TANGO 330, was deposited with the American Type Culture Collection (10801 University Boulevard, Manassas, Va. 20110-2209) on Oct. 1, 1999 and assigned PTA-816. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 330 Nucleic Acids, Polypeptides, and Modulators Thereof

As TANGO 330 form 1 was isolated from an adrenal gland library, TANGO 330, preferably form 2, nucleic acids, proteins and modulators thereof can be utilized to track and/or modulate the function of normal or dysfunctional adrenal cells and tissues. TANGO 330 nucleic acids, proteins or modulators thereof can be used to diagnose, monitor and/or treat disorders of the adrenal cortex such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma). TANGO 330 nucleic acids, proteins or modulators thereof can also be used to diagnose, monitor and/or treat disorders of the adrenal medulla such as neoplasms (e.g., pheochromocytomas, neuroblastomas, and ganglioneuromas).

As human TANGO 330 form 2 was originally identified in an astrocyte library, TANGO 330 nucleic acids, proteins, and modulators thereof can be used to track and/or modulate the proliferation, activation, maturation, development, differentiation, and/or function of glial cells e.g., astrocytes and oligodendrocytes. TANGO 330 nucleic acids, proteins and modulators thereof can be used to diagnose, monitor and/or treat glial cell-related disorders, e.g., astrocytoma and glioblastoma.

In light of the above and the fact that TANGO 330 family members have characteristics of immunoglobulin superfamily proteins which are cell surface molecules involved in signal transduction and cellular proliferation, TANGO 330 nucleic acids, proteins and modulators thereof can be utilized to track and/or modulate the development and progression of cancerous and non-cancerous cell proliferative disorders such as deregulated proliferation (such as hyperdysplasia, hyper-IgM syndrome, or lymphoproliferative disorders), cirrhosis of the liver (a condition in which scarring has overtaken normal liver regeneration processes), treatment of keloid (hypertrophic scar) formation (disfiguring of the skin in which the scarring process interferes with normal renewal), psoriasis (a common skin condition characterized by excessive proliferation of the skin and delay in proper cell fate determination), benign tumors, fibrocystic conditions, tissue hypertrophy (e.g., prostatic hyperplasia), and cancers such as neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), polycythemia vera, lymphomas (Hodgkin's disease and non-Hodgkin's diseases), multiple myelomas and Waldenström's macroglobulinemia.

Furthermore, TANGO 330 nucleic acids, proteins and modulators thereof can be utilized to track and/or modulate immune activation. For example, antagonists to TANGO 330 action, such as peptides, antibodies or small molecules that decrease or block TANGO 330 activity, e.g., binding to extracellular matrix components, e.g., integrins, or that prevent TANGO 330 signaling can be used as immune system activation blockers. In another example, agonists that mimic or partially mimic TANGO 330 activity, such as peptides, antibodies or small molecules, can be used to induce immune system activation. Antibodies may activate or inhibit the cell adhesion, proliferation and activation, and may help in treating infection, autoimmunity, inflammation, and cancer by affecting these cellular processes. TANGO 330 nucleic acids, proteins and modulators thereof can also be utilized to track and/or modulate intercellular signaling in the immune system. For example, TANGO 330 nucleic acids, proteins and modulators thereof can be used to modulate intercellular signal transduction in immune stimulation or suppression and modulate immune cell membrane adhesion to ECM components, during development, e.g., late stages of development.

As TANGO 330 exhibits homology to roundabout, which is the cellular receptor for SLIT proteins, TANGO 330 proteins, nucleic acids and modulators thereof may be used to track and/or modulate the development, activity, and maintenance of neural tissues or cells by e.g., protein-protein interactions. TANGO 330 nucleic acids, proteins and modulators thereof may also modulate neural function e.g., sensory neural cell signaling. TANGO 330 protein, nucleic acids and modulators thereof could also be useful to diagnose, monitor and/or treat neural related disorders or neural damage such as for regenerative neural repair after damage by trauma, degeneration, or inflammation, e.g., multiple sclerosis, spinal cord injuries, infarction, infection, malignancy, exposure to toxic agents, nutritional deficiency, paraneoplastic syndromes, and degenerative nerve diseases including but not limited to Alzheimer's disease, Parkinson's disease, Huntington's Chorea, amyotrophic lateral sclerosis, progressive supra-nuclear palsy, and other dementia.

As TANGO 330 proteins contain fibronectin domains, TANGO 330 nucleic acids, proteins and modulators thereof can be utilized to track and/or modulate cellular migration and invasion through the cell matrix. For example, TANGO 330 nucleic acids, proteins and modulators thereof can be used to modulate such cellular process as intracellular responses to cell adhesion including stimulation of migration, the assembly of an F-actin cytoskeleton and specialized structures called focal contacts, changes of cytoplasmic pH and calcium ion concentration, and modulation of proliferation and gene expression. Fibronectin, and thus, TANGO 330 nucleic acids, proteins and modulators thereof may also modulate cellular responses to fibronectin substrates, such responses include adhesion, migration, assembly of extracellular matrix, and signal transduction.

TANGO 330 nucleic acids and/or proteins can be utilized as markers for adrenal cells and glial cells (e.g., astrocytes and oligodendrocytes). Further, TANGO 330 nucleic acids can be utilized for chromosomal mapping, or as chromosomal markers, e.g., in radiation hybrid mapping.

### Human TANGO 437

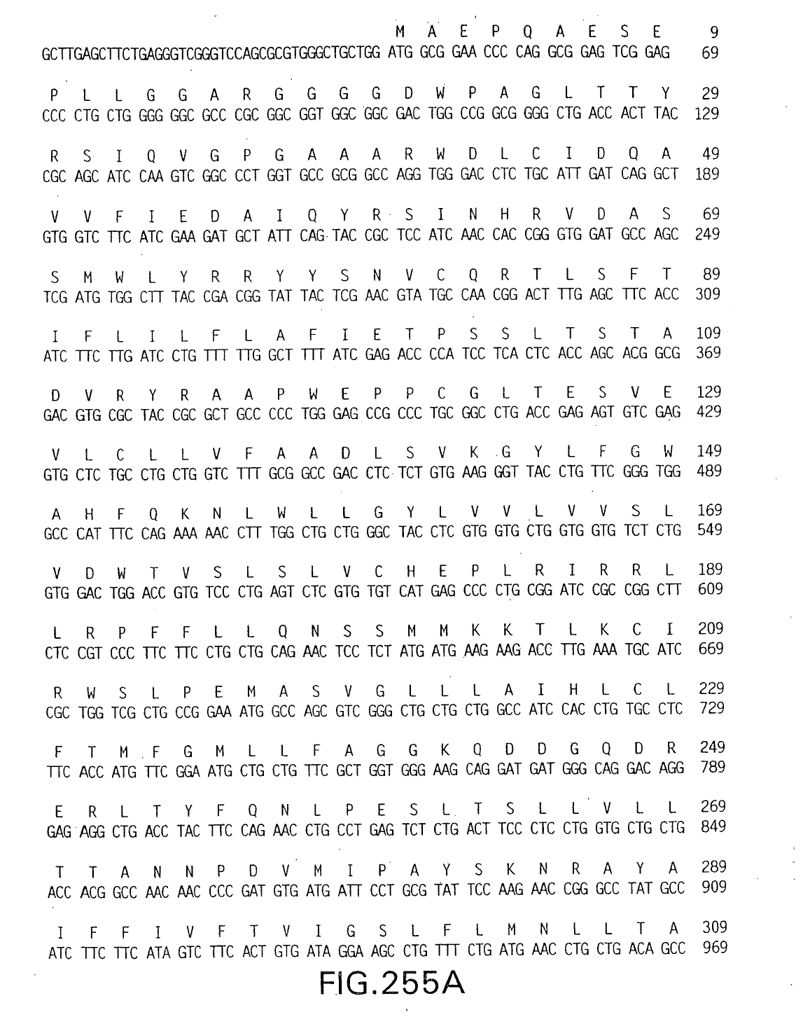

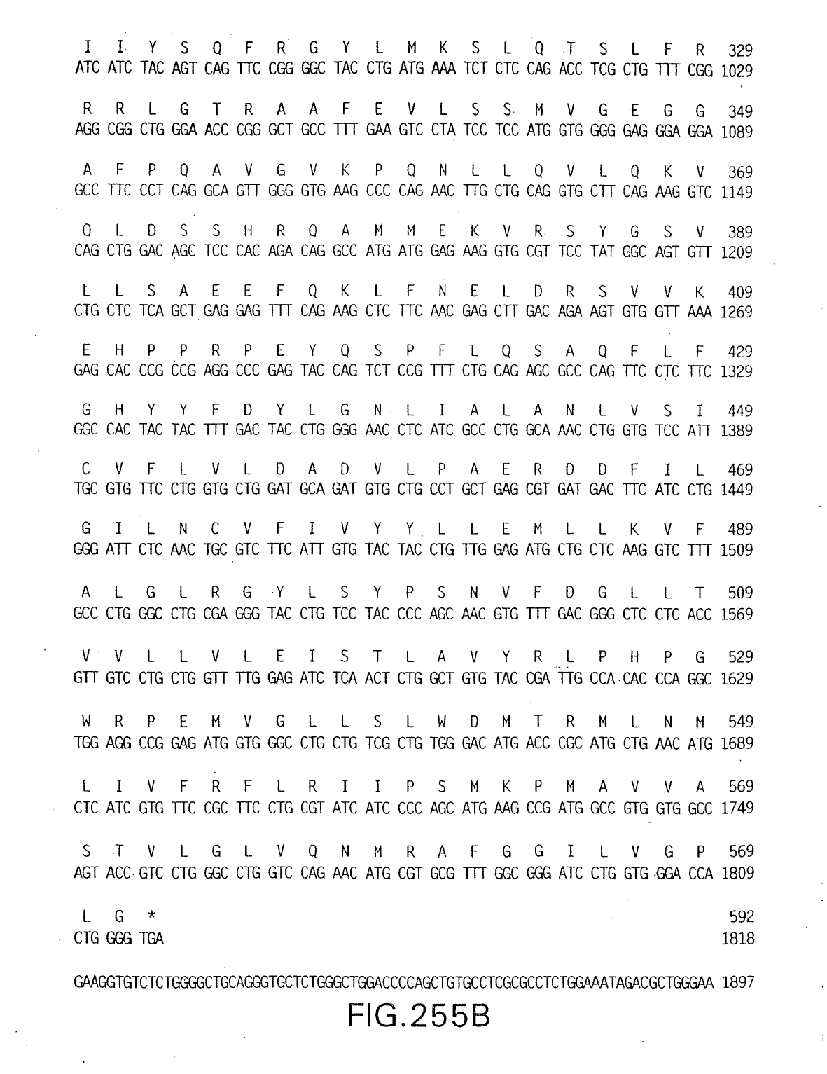

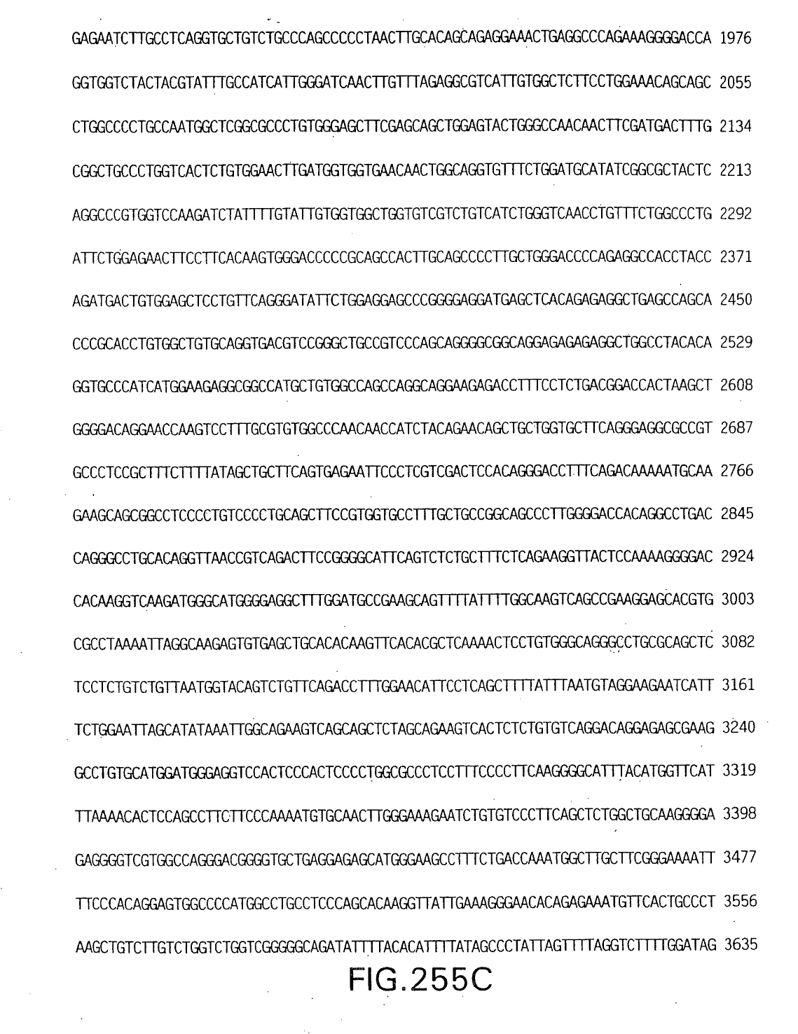

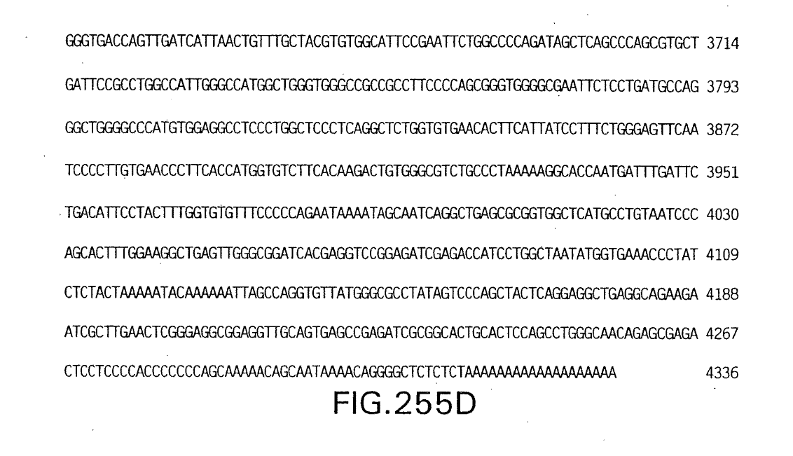

A cDNA encoding human TANGO 437 was identified by analyzing the sequences of clones present in a human mixed lymphocyte reaction library for sequences that encode wholly secreted or transmembrane proteins. This analysis led to the identification of a clone, jthLa045b02, encoding full-length human TANGO 437. The human TANGO 437 cDNA of this clone is 4336 nucleotides long (FIG. 255A-255D; SEQ ID NO:159). The open reading frame of this cDNA, nucleotides 43 to 1815, encodes a 591 amino acid transmembrane protein (SEQ ID NO: 160). The predicted molecular weight of a TANGO 437 protein without post-translational modifications is 66.5 kDa.

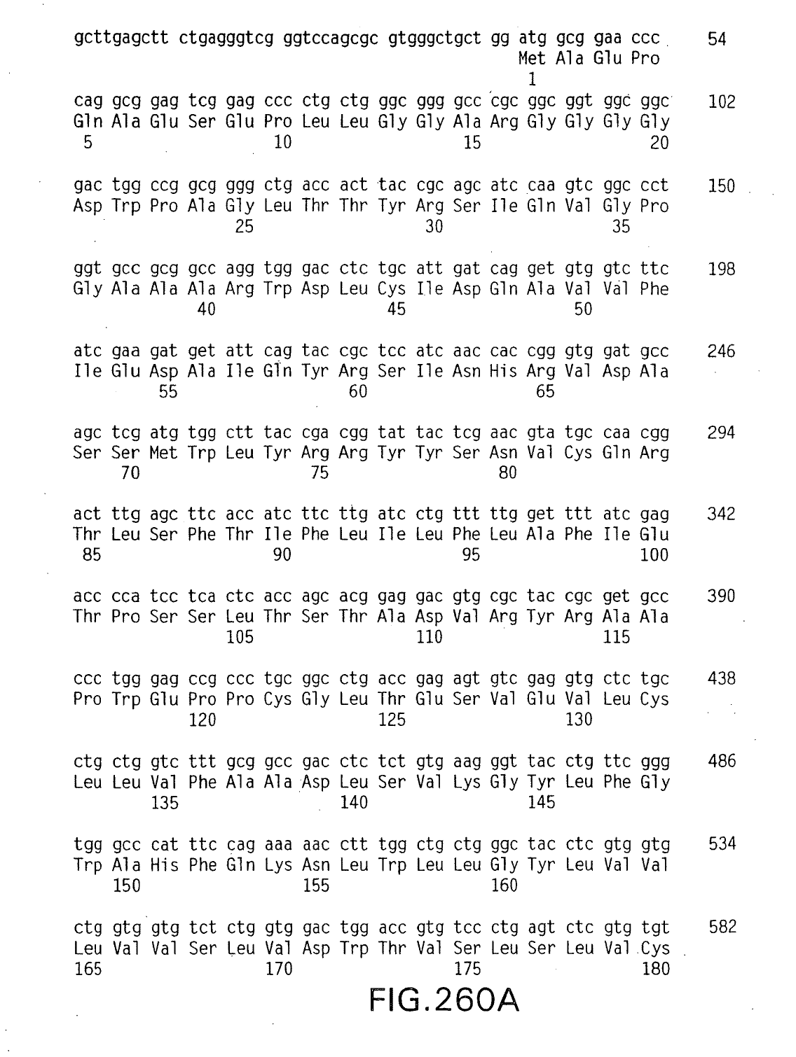

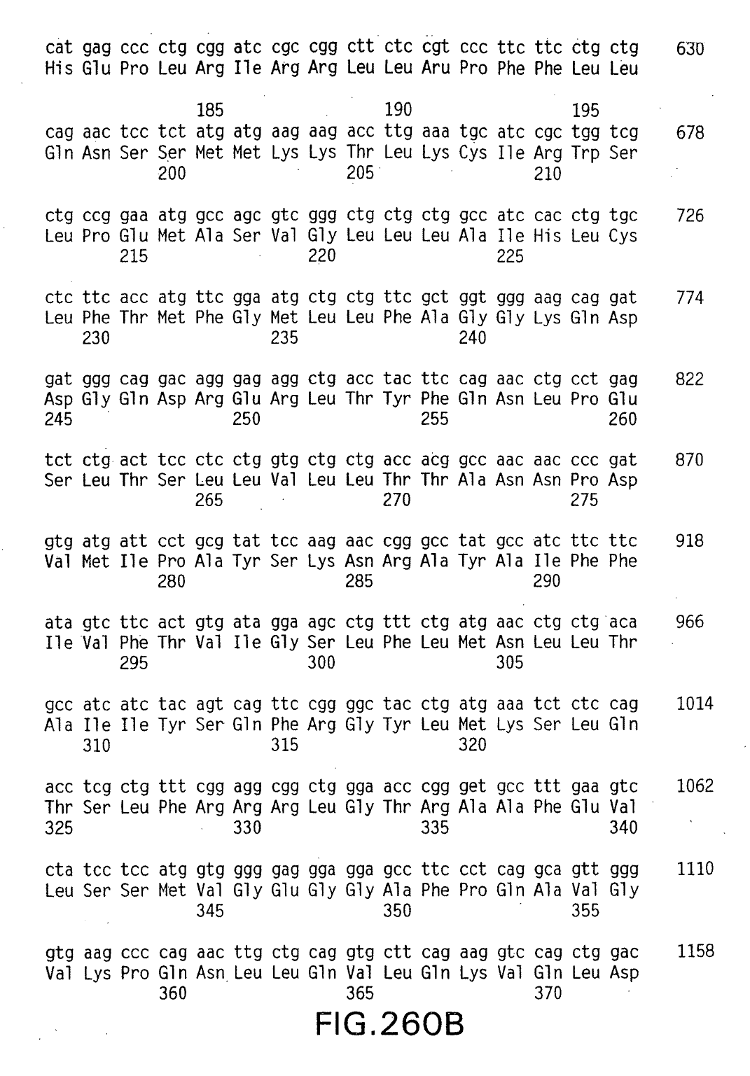

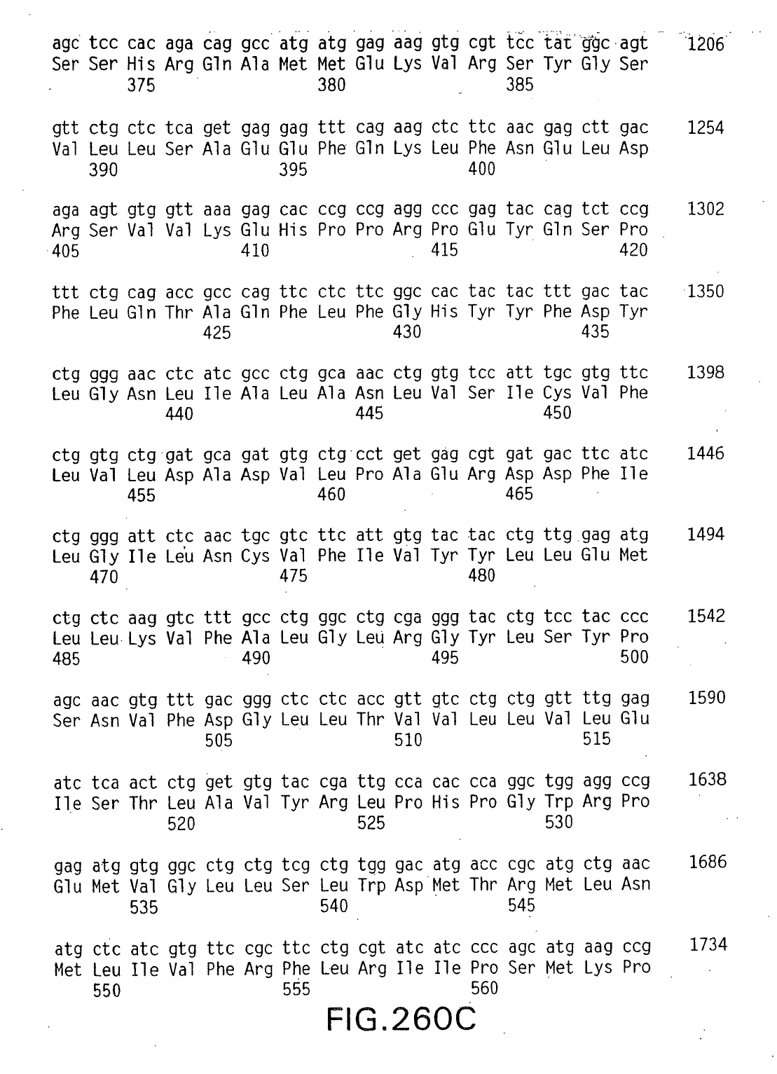

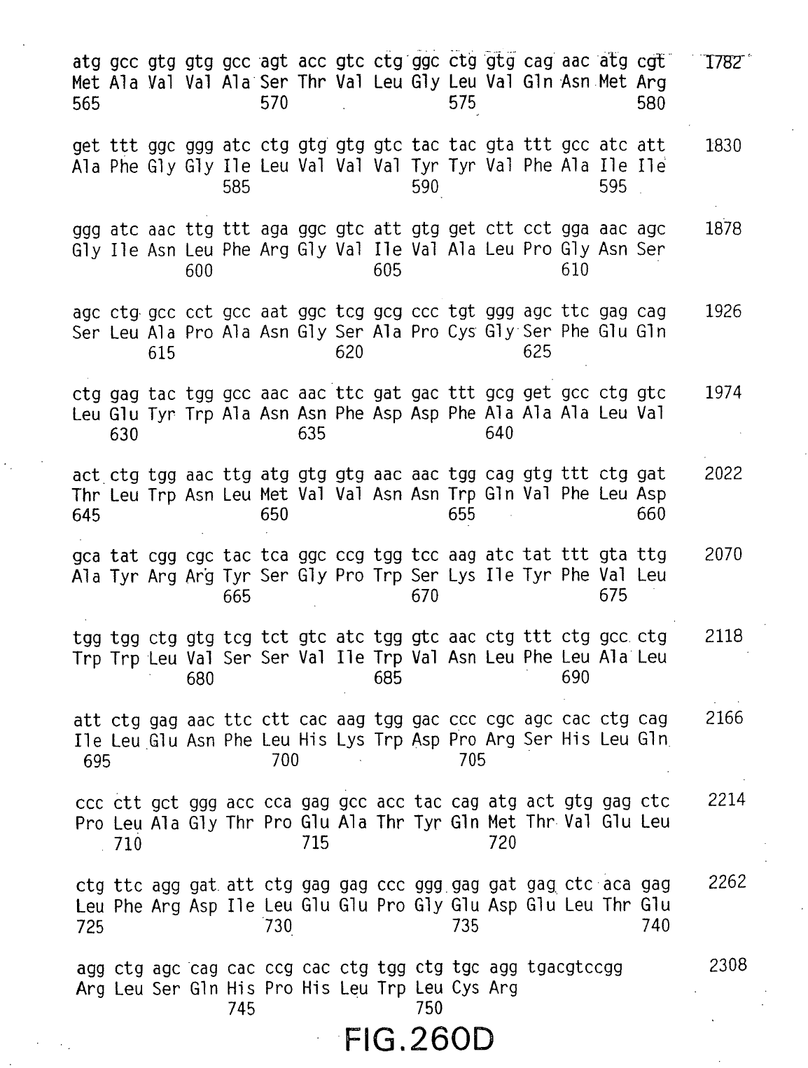

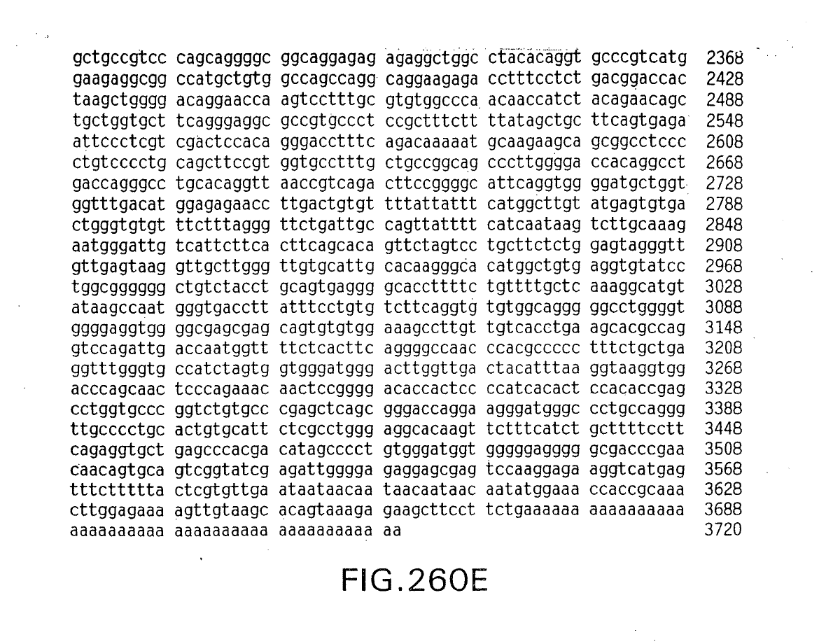

A clone encoding TANGO 437-form 2 was identified as well, the cDNA of which is 3720 nucleotides long (FIG. 260A-260E; SEQ ID NO: 163). The open reading frame of this cDNA, nucleotides 43 to 2298, encodes a 752 amino acid transmembrane protein (SEQ ID NO: 164). The predicted molecular weight of a TANGO 437 protein without post-translational modifications is 85.3 kDa.

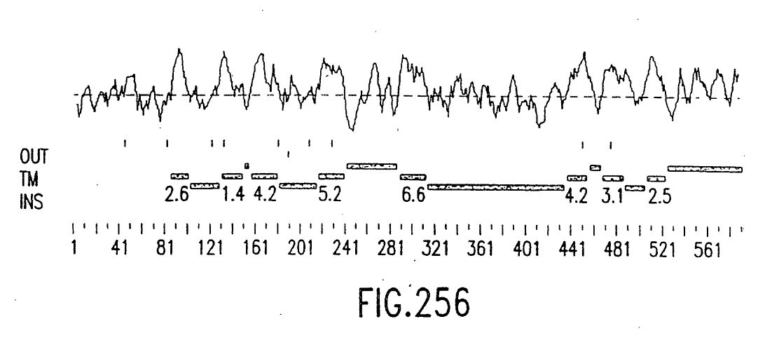

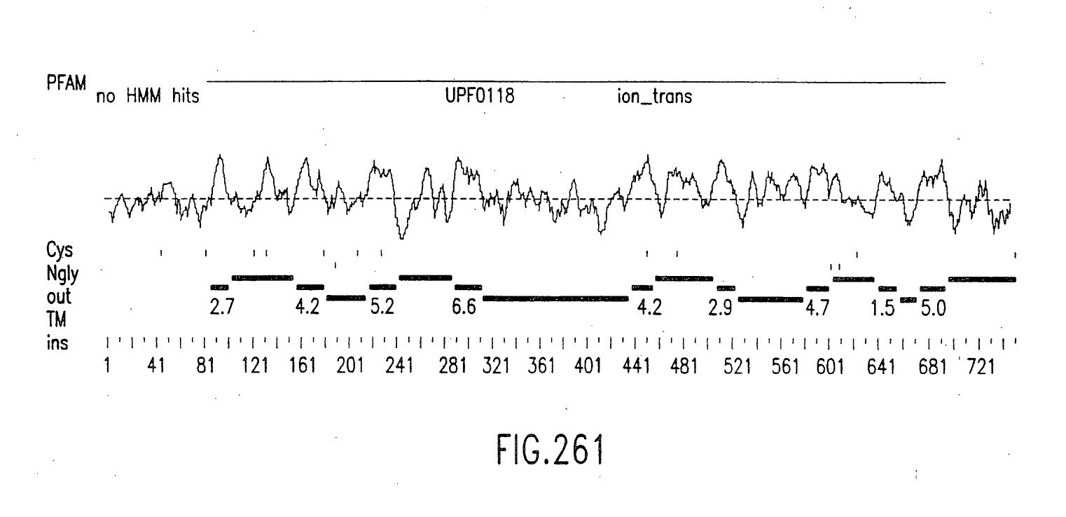

FIGS. 256 and 261 depict hydropathy plots of partial and full length human TANGO 437, respectively.

Human TANGO 437 protein is a transmembrane protein that contains extracellular domains at amino acid residues 1 to 84, 150 to 155, 241 to 287, 456 to 466, and 524 to 591, transmembrane domains at amino acid residues 85 to 101, 130 to 149, 156 to 180, 216 to 240, 288 to 312, 436 to 455, 467 to 486, and 506 to 523, and cytoplasmic domains at amino acid residues 102 to 129, 181 to 215, 313 to 435, and 487 to 505 of SEQ ID NO:160.

Alternatively, a TANGO 437 protein contains extracellular domains at amino acid residues 1 to 84, 181 to 215, 313 to 435, and 487 to 505, the following seven transmembrane domains at amino acid residues 85 to 101, 156 to 180, 216 to 240, 288 to 312, 436 to 455, 467 to 486, and 506 to 523, and cytoplasmic domains at amino acid residues 102 to 129, 241 to 287, 456 to 466, 524 to 591 of SEQ ID NO:160.

Alternatively, in another embodiment, a human TANGO 437 protein is a transmembrane protein that contains cytoplasmic domains at amino acid residues 1 to 84, 150 to 155, 241 to 287, 456 to 466, and 524 to 591, transmembrane domains at amino acid residues 85 to 101, 130 to 149, 156 to 180, 216 to 240, 288 to 312, 436 to 455, 467 to 486, and 506 to 523, and extracellular domains at amino acid residues 102 to 129, 181 to 215, 313 to 435, and 487 to 505 of SEQ ID NO:160.

Alternatively, a TANGO 437 protein contains cytoplasmic domains at amino acid residues 1 to 84, 181 to 215, 313 to 435, and 487 to 505, the following seven transmembrane domains at amino acid residues 85 to 101, 156 to 180, 216 to 240, 288 to 312, 436 to 455, 467 to 486, and 506 to 523, and extracellular domains at amino acid residues 102 to 129, 241 to 287, 456 to 466, 524 to 591 of SEQ ID NO:160.

TANGO 437-form 2 protein is a transmembrane protein that contains extracellular domains at about amino acid residues 1 to 84, 181 to 215, 313 to 435, 524 to 580, and 656 to 671, transmembrane domains at about amino acid residues 85 to 101, 130 to 149, 156 to 180, 216 to 240, 288 to 312, 436 to 455, 467 to 486, 506 to 523, 581 to 601, 639 to 655, and 672 to 694, and cytoplasmic domains at about amino acid residues 102 to 155, 241 to 287, 456 to 505, 602 to 638, and 695 to 752 of SEQ ID NO:164.

Alternatively, TANGO 437-form 2 protein contains cytoplasmic domains at about amino acid residues 1 to 84, 181 to 215, 313 to 435, 524 to 580, and 656 to 671, transmembrane domains at about amino acid residues 85 to 101, 130 to 149, 156 to 180, 216 to 240, 288 to 312, 436 to 455, 467 to 486, 506 to 523, 581 to 601, 639 to 655, and 672 to 694, and extracellular domains at about amino acid residues 102 to 155, 241 to 287, 456 to 505, 602 to 638, and 695 to 752.

In one embodiment the sequence of human TANGO 437 and human TANGO 437-form 2 open reading frames (SEQ ID NOs: 160 and 164, respectively) have a nucleotide at position 5 which is cytosine (C). In this embodiment, the cDNA contains an open reading frame encoding a polypeptide having an amino acid at position 2 that is alanine (A). In an alternative embodiment, a species variant cDNA sequence of human TANGO 437 has a nucleotide at position 5 which is thymidine (T). In this embodiment, the cDNA contains an open reading frame encoding a polypeptide having an amino acid at position 2 that is valine (V), i.e., a conservative substitution.

In one embodiment the sequence of human TANGO 437 and human TANGO 437-form 2 open reading frames (SEQ ID NOs:160 and 164, respectively) have a nucleotide at position 9 which is adenine (A). In this embodiment, the cDNA contains an open reading frame encoding a polypeptide having an amino acid at position 3 that is glutamate (E). In an alternative embodiment, a species variant cDNA sequence of human TANGO 437 has a nucleotide at position 9 which is cytosine (C). In this embodiment, the cDNA contains an open reading frame encoding a polypeptide having an amino acid at position 3 that is aspartate (D), i.e., a conservative substitution.

In one embodiment the sequence of human TANGO 437 and human TANGO 437-form 2 open reading frames (SEQ ID NOs:160 and 164, respectively) have a nucleotide at position 86 which is adenine (A). In this embodiment, the cDNA contains an open reading frame encoding a polypeptide having an amino acid at position 29 that is tyrosine (Y). In an alternative embodiment, a species variant cDNA sequence of human TANGO 437 has a nucleotide at position 86 which is thymidine (T). In this embodiment, the cDNA contains an open reading frame encoding a polypeptide having an amino acid at position 29 that is phenylalanine (F), i.e., a conservative substitution.

In one embodiment the sequence of human TANGO 437 and human TANGO 437-form 2 open reading frames (SEQ ID NOs:160 and 164, respectively) have a nucleotide at position 746 which is guanine (G). In this embodiment, the cDNA contains an open reading frame encoding a polypeptide having an amino acid at position 249 that is arginine (R). In an alternative embodiment, a species variant cDNA sequence of human TANGO 437 has a nucleotide at position 746 which is adenine (A). In this embodiment, the cDNA contains an open reading frame encoding a polypeptide having an amino acid at position 249 that is lysine (K), i.e., a conservative substitution.

Secretion assays indicate that the polypeptide encoded human TANGO 437 is not secreted and thus, a transmembrane protein. The secretion assays were performed as follows: 8×10⁵ 293T cells were plated per well in a 6-well plate and the cells were incubated in growth medium (DNMEM, 10% fetal bovine serum, penicillin/strepomycin) at 37° C., 5% CO₂ overnight. 293T cells were transfected with 2 μg of full-length TANGO 437 inserted in the pMET7 vector/well and 10 μg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16-424-54). 1 ml DMEM without methionine and cysteine with 50 μCi Trans-³⁵S (ICN Cat. # 51006) was added to each well and the cells were incubated at 37° C., 5% CO₂ for the appropriate time period. A 150 μl aliquot of conditioned medium was obtained and 150 μL of 2×SDS sample buffer was added to the aliquot. The sample was heat-inactivated and loaded on a 4-20% SDS-PAGE gel. The gel was fixed and the presence of secreted protein was detected by autoradiography.

Human TANGO 437 includes an ion transport protein-like domain at amino acids 82 to 311 and a putative permease-like domain at amino acids 284 to 591 of SEQ ID NO: 160.

Human TANGO 437 has an N-glycosylation sites with the sequence NSSM (at amino acid residues 198 to 201). Five protein kinase C phosphorylation sites are present in TANGO 437. The first has the sequence TYR (at amino acid residues 28 to 30), the second has the sequence SVK (at amino acid residues 141 to 143), the third has the sequence TLK (at amino acid residues 205 to 207), the fourth has the sequence SHR (at amino acid residues 374 to 376), and the fifth has the sequence SMK (at amino acid residues 561 to 563). TANGO 437 has five casein II kinase phosphorylation sites. The first has the sequence STAD (at amino acid residues 107 to 110), the second has the sequence SLVD (at amino acid residues 168 to 171), the third has the sequence SLPE (at amino acid residues 212 to 215), the fourth has the sequence SAEE (at amino acid residues 392 to 395), and the fifth has the sequence SLWD (at amino acid residues 539 to 542).

Seven N-myristylation sites are present in TANGO 437. The first has the sequence GGARGG (at amino acid residues 13 to 18), the second has the sequence GLTESV (at amino acid residues 123 to 128), the third has the sequence GLLLAI (at amino acid residues 220 to 225), the fourth has the sequence GTRAAF (at amino acid residues 333 to 338), the fifth has the sequence GNLIAL (at amino acid residues 438 to 443), the sixth has the sequence GILNCV (at amino acid residues 470 to 475), and the seventh has the sequence GLVQNM (at amino acid residues 574 to 579).

Human TANGO 437-form 2 includes an ion transport protein-like domain at amino acids 82 to 311 and a putative permease-like domain at amino acids 284 to 591 of SEQ ID NO:164.

Human TANGO 437-form 2 has at least one or more of the following post-translational sites: predicted N-glycosylation sites from about amino acids 198-201, 611-614, and 618-621 of SEQ ID NO: 164; predicted cAMP- and cGMP-dependent protein kinase phosphorylation site from about amino acids 663-666 of SEQ ID NO: 164; predicted protein kinase C phosphorylation sites from about amino acids 28-30, 141-143, 205-207, 374-376, 561-563, and 739-741 of SEQ ID NO: 164; predicted casein II kinase phosphorylation sites from about amino acids 107-110, 168-171, 212-215, 392-395, and 539-542 of SEQ ID NO: 164; predicted N-myristylation sites from about amino acids 13-18, 123-128, 220-225, 333-338, 438-443, 470-475, 574-579, 603-608, 619-624, and 712-717 of SEQ ID NO:164.

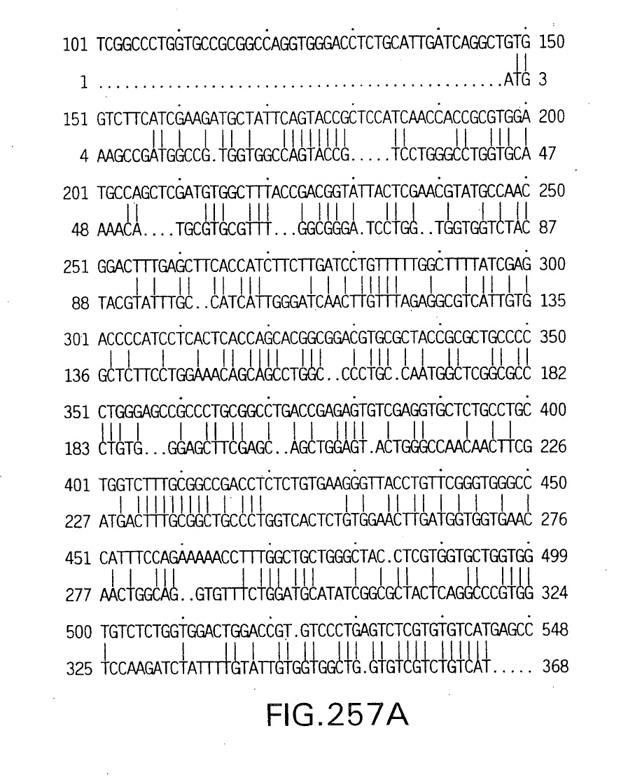

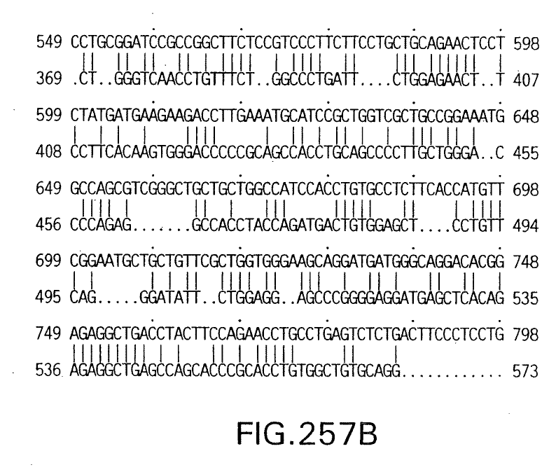

FIG. 257A-257B depicts a local alignment of the nucleotide sequence of human TANGO 437 and Gene 100 published in PCT Application No. WO98/39448 (V59610). Nucleic acids 101 to 798 of the nucleotide sequence of the coding region of human TANGO 437 and nucleic acids 1 to 573 of the nucleotide sequence of Gene 100 are 54.6% identical. Nucleic acids 1851 to 3679 of the full-length nucleotide sequence of TANGO 437 and nucleic acids 1 to 1751 of the nucleotide sequence of Gene 100 are 74.1% identical.

Clone 437, which encodes human TANGO 437, was deposited with the American Type Culture Collection (10801 University Boulevard, Manassas, Va. 20110-2209) on Oct. 1, 1999 and assigned PTA-816. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 437 Nucleic Acids, Polypeptides, and Modulators Thereof

As TANGO 437 was originally found in a mixed lymphocyte reaction cell library, TANGO 437 nucleic acids, proteins, and modulators thereof can be used to track and/or modulate the proliferation, development, maturation, differentiation, activity and/or function of immune cells, e.g. B-cells, dendritic cells, natural killer cells and monocytes, and/or immune function. TANGO 437 nucleic acids, proteins and modulators thereof can be utilized to track and/or modulate immune-related processes such as the host immune response. For example, TANGO 437 nucleic acids, proteins, and modulators thereof can be used to modulate the host immune response by modulating the formation of and/or binding to immune complexes, detection and defense against surface antigens and bacteria, and immune surveillance for rapid removal or pathogens.

TANGO 437 has significant homology with Gene 100, which is expressed primarily in hepatocellular tumors and encodes a secreted human protein. As such, TANGO 437 nucleic acids, proteins and modulators thereof can be used to diagnose, monitor, modulate and/or treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g. hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

TANGO 437 nucleic acids, proteins and modulators thereof can be utilized to diagnose, monitor, modulate and/or treat immune disorders that include, but are not limited to, immune proliferative disorders (e.g., carcinoma, lymphoma, e.g., follicular lymphoma), and disorders associated with fighting pathogenic infections, (e.g., bacterial (e.g., chlamydia) infection, parasitic infection, and viral infection (e.g., HSV or HIV infection)), and pathogenic disorders (e.g., immunodeficiency disorders, such as HIV), autoimmune disorders, such as arthritis, graft rejection (e.g., allograft rejection), multiple sclerosis, Grave's disease, or Hashimoto's disease, T cell disorders (e.g., AIDS) and inflammatory disorders, such as septicemia, cerebral malaria, inflammatory bowel disease, arthritis (e.g., rheumatoid arthritis, ostcoarthritis), and allergic inflammatory disorders (e.g. asthma, psoriasis), apoptotic disorders (e.g., rheumatoid arthritis, systemic lupus erythematosus, insulin-dependent diabetes mellitus), cytotoxic disorders, septic shock, and cachexia.

TANGO 437 nucleic acids, proteins and modulators thereof can be utilized to track and/or modulate intracellular signaling. TANGO 437 nucleic acids, proteins and modulators thereof can also be utilized to track and/or modulate immune activation. For example, antagonists to TANGO 437 action, such as peptides, antibodies or small molecules that decrease or block TANGO 437 activity, e.g., binding to extracellular matrix components, e.g., integrins, or that prevent TANGO 437 signaling, can be used as immune system activation blockers. In another example, agonists that mimic or partially mimic TANGO 437 activity, such as peptides, antibodies or small molecules, can be used to induce immune system activation. Antibodies may activate or inhibit the cell adhesion, proliferation and activation, and may help in treating infection, autoimmunity, inflammation, and cancer by affecting these cellular processes. Further, TANGO 437 nucleic acids, proteins and modulators thereof can be utilized to track and/or modulate intercellular signaling in the immune system, e.g., modulate intercellular signal transduction in immune stimulation or suppression and modulate immune cell membrane adhesion to ECM components, during development, e.g., late stages of development.

As TANGO 437 contains an ion transport protein domain, TANGO 437 nucleic acids, proteins and modulators thereof can be used track and/or modulate ion transport (e.g., sodium, calcium or potassium transport). TANGO 437 nucleic acids, proteins and modulators thereof can be utilized to diagnose, monitor, modulate and/or treat disorders associated with aberrant ion transport. Examples of such disorders include, but are not limited to, pulmonary disorders (e.g., cystic fibrosis) and renal disorders.

As TANGO 437 contains a cell cycle protein-like domain, TANGO 437 nucleic acids, proteins and modulators thereof can be used track and/or modulate cell cycle e.g., cell cycle progression. TANGO 437 nucleic acids, proteins and modulators thereof can, for example, be used diagnose, monitor, modulate and/or treat disorders associated with aberrant cell cycle progression including various types of cancer. Examples of types of cancers include benign tumors, neoplasms or tumors (such as carcinomas, sarcomas, adenomas or myeloid lymphoma tumors, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leimyosarcoma, rhabdotheliosarcoma, colon sarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hematoma, bile duct carcinoma, melanoma, choriocarcinoma, semicoma, embryonal carcinoma, Wilms' tumor, cervical cancer, testicular tumor, lung carcinoma, small cell carcinoma, bladder carcinoma, epithelial carcinoma, glioma, astrocytoma, medulloblastoma, craniopharyngioma, ependynoma, pinealoma, hemangioblastoma, retinoblastoma), leukemias (e.g. acute lymphocytic leukemia), acute myelocytic leukemia (myelolastic, promyelocytic, myelomonocytic, monocytic and erythroleukemia), chronic leukemias (chronic myelocytic (granulocytic) leukemia and chronic lymphocytic leukemia), polycythemia vera, lymphomas (Hodgkin's disease and non-Hodgkin's diseases), multiple myelomas and Waldenstrom's macroglobulinemia.

TANGO 437 nucleic acids and/or proteins can be utilized as markers for immune cells (e.g., T cells, B cells, natural killer cells, mast cells, and dendritic cells). Further, TANGO 437 nucleic acids can be utilized for chromosomal mapping, or as chromosomal markers, e.g., in radiation hybrid mapping.

### Human TANGO 480

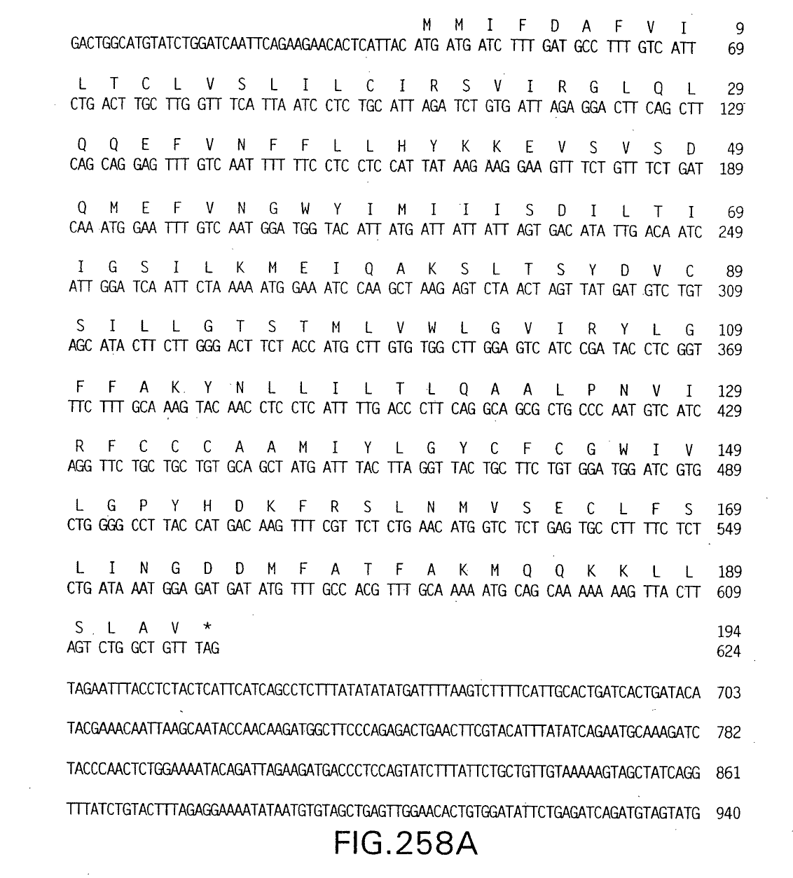

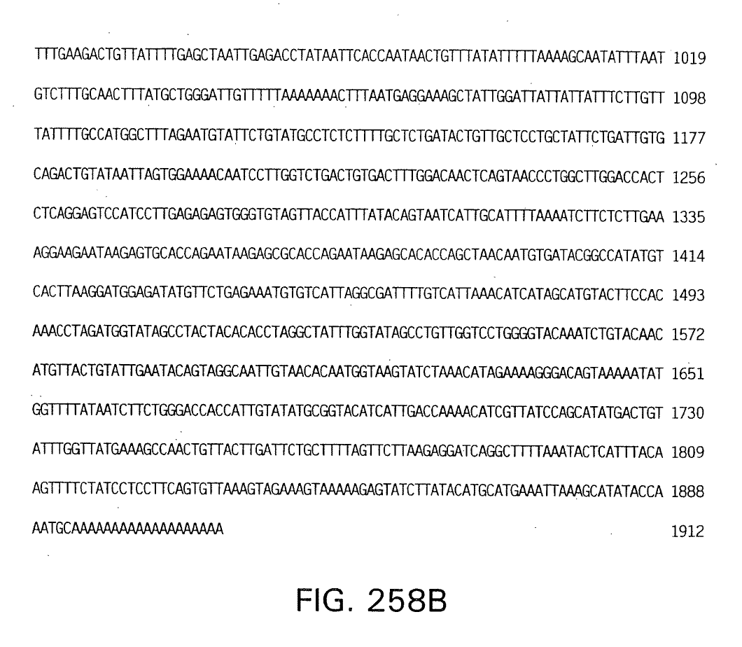

A cDNA encoding human TANGO 480 was identified by analyzing the sequences of clones present in a human keratinocyte library for sequences that encode wholly secreted or transmembrane proteins. This analysis led to the identification of a clone, jthka173a09, encoding full-length human TANGO 480. The human TANGO 480 cDNA of this clone is 1912 nucleotides long (FIG. 258A-258B; SEQ ID NO:161). The open reading frame of this cDNA, nucleotides 43 to 621, encodes a 193 amino acid transmembrane protein (SEQ ID NO:162).

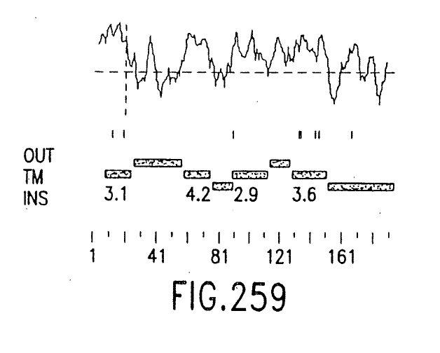

FIG. 259 depicts a hydropathy plot of human TANGO 480. The signal peptide prediction program SIGNALP (Nielsen et al., 1997, Protein Engineering 10:1-6) predicted that human TANGO 480 includes a 19 amino acid signal peptide (amino acid 1 to amino acid 19 of SEQ ID NO: 162) preceding the mature TANGO 480 protein corresponding to amino acid 20 to amino acid 193. The molecular weight of a TANGO 480 protein without post-translational modification is 22.0 kDa, and after cleavage of the signal peptide the molecular weight of TANGO 480 is 19.9 kDa.

Human TANGO 480 protein is a transmembrane protein that contains extracellular domains at amino acid residues 20 to 56 and 113 to 127, transmembrane domains at amino acid residues 55 to 74, 88 to 112, and 128 to 150, and cytoplasmic domains at amino acid residues 75 to 87 and 151 to 193 of SEQ ID NO:162.

In instances wherein the signal peptide is not cleaved, a human TANGO 480 protein is a transmembrane protein that contains extracellular domains at amino acid residues 1 to 56 and 113 to 127, transmembrane domains at amino acid residues 55 to 74, 88 to 112, and 128 to 150, and cytoplasmic domains at amino acid residues 75 to 87 and 151 to 193 of SEQ ID NO: 162.

Alternatively, in another embodiment, a human TANGO 480 protein is a transmembrane protein that contains cytoplasmic domains at amino acid residues 20 to 56 and 113 to 127, transmembrane domains at amino acid residues 55 to 74, 88 to 112, and 128 to 150, and extracellular domains at amino acid residues 75 to 87 and 151 to 193 of SEQ ID NO:162.

In one embodiment a cDNA sequence of human TANGO 480 has a nucleotide at position 7 which is adenine (A). In this embodiment, the cDNA contains an open reading frame encoding a polypeptide having an amino acid at position 3 that is isoleucine (I). In an alternative embodiment, a species variant cDNA sequence of human TANGO 480 has a nucleotide at position 7 which is guanine (G). In this embodiment, the cDNA contains an open reading frame encoding a polypeptide having an amino acid at position 3 that is valine (V), i.e., a conservative substitution.

In another embodiment a cDNA sequence of human TANGO 480 has a nucleotide at position 11 which is thymidine (T). In this embodiment, the cDNA contains an open reading frame encoding a polypeptide having an amino acid at position 4 that is phenylalanine (F). In an alternative embodiment, a species variant cDNA sequence of human TANGO 480 has a nucleotide at position 11 which is adenine (A). In this embodiment, the cDNA contains an open reading frame encoding a polypeptide having an amino acid at position 4 that is tyrosine (Y), i.e., a conservative substitution.

In another embodiment a cDNA sequence of human TANGO 480 has a nucleotide at position 13 which is guanine (G). In this embodiment, the cDNA contains an open reading frame encoding a polypeptide having an amino acid at position 5 that is aspartate (D). In an alternative embodiment, a species variant cDNA sequence of human TANGO 480 has a nucleotide at position 13 which is adenine (A). In this embodiment, the cDNA contains an open reading frame encoding a polypeptide having an amino acid at position 5 that is asparagine (N), i.e., a conservative substitution.

In another embodiment a cDNA sequence of human TANGO 480 has a nucleotide at position 389 which is guanine (G). In this embodiment, the cDNA contains an open reading frame encoding a polypeptide having an amino acid at position 13 that is arginine (R). In an alternative embodiment, a species variant cDNA sequence of human TANGO 480 has a nucleotide at position 389 which is adenine (A). In this embodiment, the cDNA contains an open reading frame encoding a polypeptide having an amino acid at position 130 that is lysine (K), i.e., a conservative substitution.

Secretion assays indicate that the polypeptide encoded by human TANGO 480 is not secreted and thus, likely a transmembrane protein. The secretion assays were performed as follows: 8×10⁵ 293T cells were plated per well in a 6-well plate and the cells were incubated in growth medium (DMEM, 10% fetal bovine serum, penicillin/strepomycin) at 37° C., 5% CO₂ overnight. 293T cells were transfected with 2 μg of full-length TANGO 480 inserted in the pMET7 vector/well and 10 μg LipofectAMINE (GIBCO/BRL Cat. # 18324-012)/well according to the protocol for GIBCO/BRL LipofectAMINE. The transfectant was removed 5 hours later and fresh growth medium was added to allow the cells to recover overnight. The medium was removed and each well was gently washed twice with DMEM without methionine and cysteine (ICN Cat. # 16-424-54). Next, 1 ml DMEM without methionine and cysteine with 50 μCi Trans-³⁵S (ICN Cat. # 51006) was added to each Well and the cells were incubated at 37° C., 5% CO₂ for the appropriate time period. A 150 μl aliquot of conditioned medium was obtained and 150 μl of 2×SDS sample buffer was added to the aliquot. The sample was heat-inactivated and loaded on a 4-20% SDS-PAGE gel. The gel was fixed and the presence of secreted protein was detected by autoradiography.

Human TANGO 480 has two casein II kinase phosphorylation sites. The first has the sequence SVSD (at amino acid residues 46 to 49) and the second has the sequence TSYD (at amino acid residues 84 to 87).

Clone 480, which encodes human TANGO 480, was deposited with the American Type Culture Collection (10801 University Boulevard, Manassas, Va. 20110-2209) on Oct. 1, 1999 and assigned PTA-816. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Uses of TANGO 480 Nucleic Acids, Polypeptides, and Modulators Thereof

As TANGO 480 was originally found in a human keratinocyte library, TANGO 480 nucleic acids, proteins, and modulators thereof can be used to track and/or modulate the proliferation, development, maturation, differentiation, activity and/or function of keratinocytes. TANGO 480 nucleic acids, proteins, and modulators thereof can be utilized to track and/or modulate keratinocyte-related processes. TANGO 480 nucleic acids, proteins and modulators thereof can be utilized to diagnose, monitor, modulate and/or treat keratinocyte disorders that include, but are not limited to, keratinocyte proliferative disorders (e.g., squamous cell carcinoma), keratitis, keratoacanthoma, keratoconjunctivitis, keratoconus, keratoderma blennorrhagica, keratomalacia, keratopathy, keratinous cysts, and keratosis.

Keratinocyte growth factor (KGF) is a fibroblast growth factor that acts specifically on epithelial cells in a paracrine mode and mediates epithelial growth and differentiation. TANGO 480 nucleic acids, proteins, and modulators thereof may thus be used to track and/or modulate the activity of human keratinocyte (HKc) growth and/or differentiation.

Since high affinity muscarinic acetylcholine receptors (mAChR) have been found on keratinocyte cell surfaces at high density, TANGO 480 nucleic acids, proteins, and modulators thereof can be used to track and/or modulate the activity of such muscarinic acetylcholine receptors.

TANGO 480 nucleic acids, proteins, and modulators thereof can be used to modulate the activity of the cell cycle arrest program which is activated by TGF-beta in human keratinocytes.

TANGO 480 nucleic acids, proteins, and modulators thereof can be used to track and/or modulate the activity of the calcium sensing receptor (CaR) in keratinocytes which may be involved in the signaling of calcium-induced differentiation.

TANGO 480 nucleic acids, proteins, and modulators thereof can be used to track and/or modulate the activity of the GlcCer synthase (GCS) which is up-regulated at the transcriptional level during keratinocyte differentiation.

TANGO 480 nucleic acids, proteins, and modulators thereof can be used to track and/or modulate the activity of the cyclic AMP phosphodiesterase (PDE) type 4 PDE isogenes which are expressed in keratinocytes to a different degree, the expression of each of which is modulated by intracellular levels of cAMP.

Platelet-derived growth factor (PDGF) can promote tumor growth by inducing angiogenesis and stroma formation. Thus, TANGO 480 nucleic acids, proteins, and modulators thereof may be used to track and/or modulate the activity of PDGF, a major factor activated in wound healing.

TANGO 480 nucleic acids, proteins and modulators thereof can be utilized to track and/or modulate intracellular signaling. TANGO 480 nucleic acids, proteins and modulators thereof can also be utilized to track and/or modulate keratinocyte activity and/or function. For example, antagonists to TANGO 480 action, such as peptides, antibodies or small molecules that decrease or block TANGO 480 activity, e.g., binding to extracellular matrix components, e.g., integrins, or that prevent TANGO 480 signaling, can be used as keratinocyte activation blockers. In another example, agonists that mimic or partially mimic TANGO 480 activity, such as peptides, antibodies or small molecules, can be used to induce keratinocyte activation. Antibodies may activate or inhibit the cell adhesion, proliferation and activation, and may help in treating keratinocyte associated disorders by affecting these cellular processes. Further, TANGO 480 nucleic acids, proteins and modulators thereof can be utilized to track and/or modulate intercellular signaling between keratinocytes.

TANGO 480 nucleic acids and/or proteins can be utilized as markers for keratinocytes. Further, TANGO 480 nucleic acids can be utilized for chromosomal mapping, or as chromosomal markers, e.g., in radiation hybrid mapping.

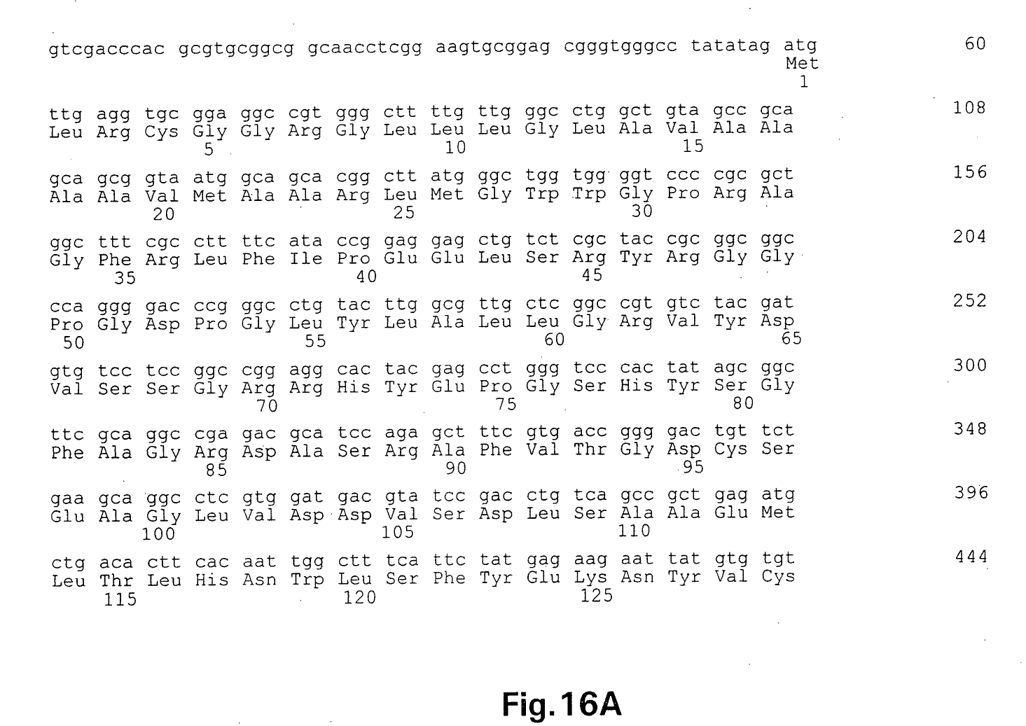

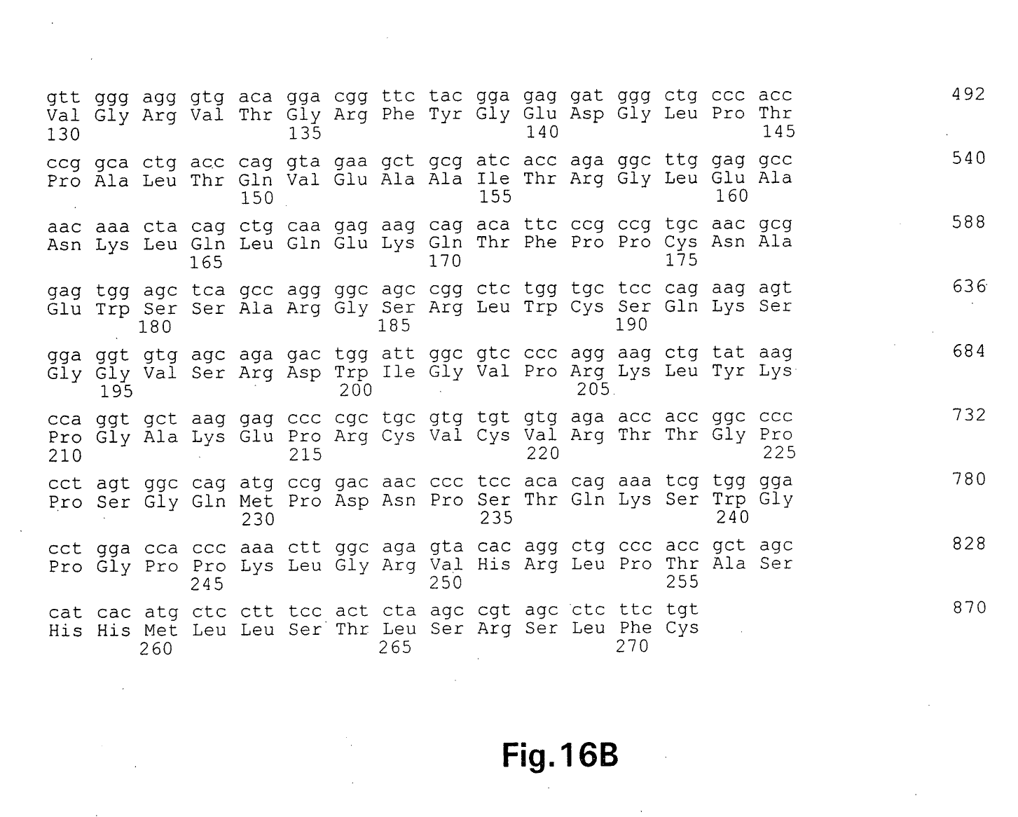

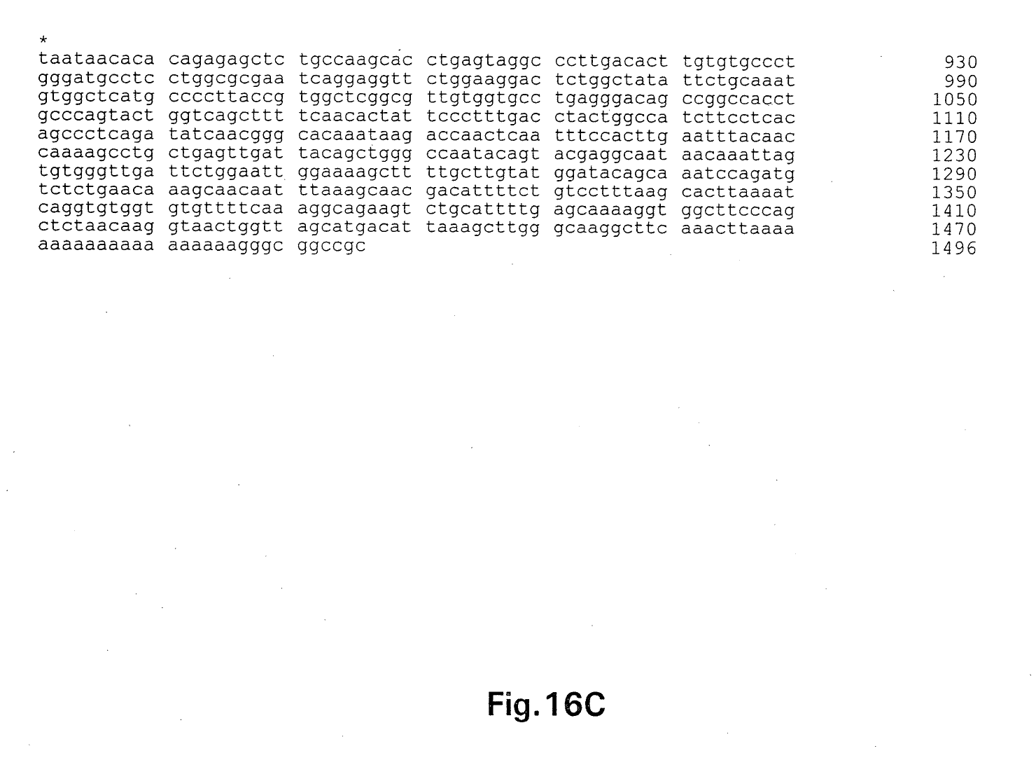

TABLE 1
Summary of Nucleotide Sequence Information of INTERCEPT 258, INTERCEPT 307
and INTERCEPT 340, MANGO 003, MANGO 245, MANGO 346, MANGO 347, MANGO 349,
and MANGO 511, and TANGO 128, TANGO 136, TANGO 140, TANGO 176, TANGO 197,
TANGO 201, TANGO 204, TANGO 206, TANGO 209, TANGO 212, TANGO 213, TANGO 214,
TANGO 216, TANGO 221, TANGO 222, TANGO 223, TANGO 224, TANGO 244, TANGO 246,
TANGO 253, TANGO 257, TANGO 261, TANGO 262, TANGO 266, TANGO 267, TANGO 272,
TANGO 275, TANGO 295, TANGO 315, TANGO 330, TANGO 339, TANGO 354, TANGO 358,
TANGO 361, TANGO 365, TANGO 368, TANGO 369, TANGO 378, TANGO 383,
TANGO 437, TANGO 480, and TANGO 499 Nucleic Acids.

| GENE FIGURE | (OPEN READING FRAME) | | | | |
|---|---|---|---|---|---|
| and cDNA | POLYPEPTIDE ATCC ACCESSION NUMBER | | | | |
| m TANGO 136 | FIG. 1 | (89-1813 b.p.) | | | |
| 1813 base pair (b.p.); | | | | | |
| SEQ ID NO: 1 | 575 amino acids (a.a.); | | | | |
| SEQ ID NO: 2 | | | | | |
| h TANGO 136 | FIG. 3 | (541-2679 b.p.) | | | |
| 2679 b.p.; | | | | | |
| SEQ ID NO: 3 | 713 a.a.; | | | | |
| SEQ ID NO: 4 | 98880 | | | | |
| h TANGO 128 | FIG. 11 | (289-1325 b.p.) | | | |
| 2839 b.p.; | | | | | |
| SEQ ID NO: 5 | 345 a.a.; | | | | |
| SEQ ID NO: 6 | 98999 | | | | |
| h TANGO 140-1 | FIG. 12 | (2-619 b.p.) | | | |
| 1550 b.p.; | | | | | |
| SEQ ID NO: 7 | 206 a.a.; | | | | |
| SEQ ID NO: 8 | 98999 | | | | |
| h TANGO 140-2 | FIG. 13 | (1-594 b.p.) | | | |
| 3385 b.p.; | | | | | |
| SEQ ID NO: 9 | 198 a.a.; | | | | |
| SEQ ID NO: 10 | | | | | |
| h TANGO 197 | FIG. 14 | (213-1214 b.p.) | | | |
| 2272 b.p.; | | | | | |
| SEQ ID NO: 11 | 333 a.a.; | | | | |
| SEQ ID NO: 12 | 98999 | | | | |
| h TANGO 212 | FIG. 15 | (269-1930 b.p.) | | | |
| 2435 b.p.; | | | | | |
| SEQ ID NO: 13 | 553 a.a.; | | | | |
| SEQ ID NO: 14 | 202171 | | | | |
| h TANGO 213 | FIG. 16 | (58-873 b.p.) | | | |
| 1496 b.p.; | | | | | |
| SEQ ID NO: 15 | 271 a.a.; | | | | |
| SEQ ID NO: 16 | 98965 | | | | |
| h TANGO 224, | | | | | |
| | | | form 1 | FIG. 17 | (67-1443 b.p.) |
| 2689 b.p.; | | | | | |
| SEQ ID NO: 17 | 480 a.a.; | | | | |
| SEQ ID NO: 18 | 98966 | | | | |
| h TANGO 224, | | | | | |
| | | | form 2 | FIG. 37 | (67-2690 b.p.) |
| 2691 b.p.; | | | | | |
| SEQ ID NO: 19 | 874 a.a.; | | | | |
| SEQ ID NO: 20 | | | | | |
| m TANGO 128 | FIG. 33 | (211-750 b.p.) | | | |
| 764 b.p.; | | | | | |
| SEQ ID NO: 21 | 179 a.a.; | | | | |
| SEQ ID NO: 22 | | | | | |
| m TANGO 197 | FIG. 34 | (3-1145 b.p.) | | | |
| 4417 b.p.; | | | | | |
| SEQ ID NO: 23 | 381 a.a.; | | | | |
| SEQ ID NO: 24 | | | | | |
| m TANGO 212 | FIG. 35 | (180-1179 b.p.) | | | |
| 1180 b.p.; | | | | | |
| SEQ ID NO: 25 | 334 a.a.; | | | | |
| SEQ ID NO: 26 | | | | | |
| m TANGO 213 | FIG. 36 | (41-616 b.p.) | | | |
| 2154 b.p.; | | | | | |
| SEQ ID NO 27: | 192 a.a.; | | | | |
| SEQ ID NO: 28 | | | | | |
| rat TANGO 213 | FIG. 38 | (FIG. 38 b.p.) | | | |
| 455 b.p.; | | | | | |
| SEQ ID NO: 29 | ; | | | | |
| SEQ ID NO: 30 | | | | | |
| h TANGO 214 | | | | | |
| | | | (HtrA-2) | FIG. 39 | (222-1580 b.p.) |
| 2577 b.p.; | | | | | |
| SEQ ID NO: 31 | 453 a.a.; | | | | |
| SEQ ID NO: 32 | 98899 | | | | |
| m TANGO 214 | | | | | |
| | | | (HtrA-2) | FIG. 44 | (268-1311 b.p.) |
| 1563 b.p.; | | | | | |
| SEQ ID NO: 33 | 349 a.a.; | | | | |
| SEQ ID NO: 34 | | | | | |
| h TANGO 221 | FIG. 45 | (6-716 b.p.) | | | |
| 1061 b.p.; | | | | | |
| SEQ ID NO: 35 | 237 a.a.; | | | | |
| SEQ ID NO: 36 | 207044 | | | | |
| h TANGO 222 | FIG. 47 | (33-434 b.p.) | | | |
| 745 b.p.; | | | | | |
| SEQ ID NO: 37 | 134 a.a.; | | | | |
| SEQ ID NO: 38 | 207043 | | | | |
| h TANGO 176 | FIG. 49 | (101-1528 b.p.) | | | |
| 16976 b.p.; | | | | | |
| SEQ ID NO: 39 | 476 a.a.; | | | | |
| SEQ ID NO: 40 | 207042 | | | | |
| m TANGO 176 | FIG. 51 | (49-1524 b.p.) | | | |
| 1904; | | | | | |
| SEQ ID NO: 41 | 492 a.a.; | | | | |
| SEQ ID NO: 42 | | | | | |
| m TANGO 201 | FIG. 52 | (60-1508 b.p.) | | | |
| 1758 b.p.; | | | | | |
| SEQ ID NO: | 483 a.a.; | | | | |
| SEQ ID NO: 44 | | | | | |
| h TANGO 201 | FIG. 54 | (179-1387 b.p.) | | | |
| 2252 b.p.; | | | | | |
| SEQ ID NO: 45 | 403 a.a.; | | | | |
| SEQ ID NO: 46 | 207081 | | | | |
| h TANGO 223 | FIG. 59 | (30-770 b.p.) | | | |
| 1473 b.p.; | | | | | |
| SEQ ID NO: 47 | 247 a.a.; | | | | |
| SEQ ID NO: 48 | 207081 | | | | |
| m TANGO 223 | FIG. 62 | (5-694 b.p.) | | | |
| 854 b.p.; | | | | | |
| SEQ ID NO: 49 | 230 a.a.; | | | | |
| SEQ ID NO: 50 | | | | | |
| h TANGO 216 | FIG. 63 | (307-1770 b.p.) | | | |
| 3677 b.p.; | | | | | |
| SEQ ID NO: 51 | 488 a.a.; | | | | |
| SEQ ID NO 52: | 207176 | | | | |
| m TANGO 216 | FIG. 64 | (149-1609 b.p.) | | | |
| 3501 b.p.; | | | | | |
| SEQ ID NO: 53 | 487 a.a.; | | | | |
| SEQ ID NO: 54 | | | | | |
| h TANGO 261 | FIG. 67 | (6-761 b.p.) | | | |
| 969 b.p.; | | | | | |
| SEQ ID NO: 55 | 252 a.a.; | | | | |
| SEQ ID NO: 56 | 207176 | | | | |
| m TANGO 261 | FIG. 68 | (2-652 b.p.) | | | |
| 1713 b.p.; | | | | | |
| SEQ ID NO: 57 | 217 a.a.; | | | | |
| SEQ ID NO: 58 | | | | | |
| h TANGO 262 | FIG. 71 | (322-999 b.p.) | | | |
| 1682 b.p.; | | | | | |
| SEQ ID NO: 59 | 226 a.a.; | | | | |
| SEQ ID NO: 60 | 207176 | | | | |
| m TANGO 262 | FIG. 72 | (89-766 b.p.) | | | |
| 1415 b.p.; | | | | | |
| SEQ ID NO: 61 | 226 a.a.; | | | | |
| SEQ ID NO: 62 | | | | | |
| h TANGO 266 | FIG. 76 | (49-363 b.p.) | | | |
| 1422 b.p.; | | | | | |
| SEQ ID NO: 63 | 105 a.a.; | | | | |
| SEQ ID NO: 64 | 207176 | | | | |
| h TANGO 267 | FIG. 79 | (161-2494 b.p.) | | | |
| 2925 b.p.; | | | | | |
| SEQ ID NO: 65 | 778 a.a.; | | | | |
| SEQ ID NO: 66 | 207176 | | | | |
| h TANGO 253 | FIG. 84 | (188-916 b.p.) | | | |
| 1339 b.p.; | | | | | |
| SEQ ID NO: 67 | 243 a.a.; | | | | |
| SEQ ID NO: 68 | 207222 | | | | |
| m TANGO 253 | FIG. 86 | (135-863 b.p.) | | | |
| 1263 b.p.; | | | | | |
| SEQ ID NO: 69 | 406 a.a. | | | | |
| SEQ ID NO: 70 | 207215 | | | | |
| h TANGO 257 | FIG. 92 | (88-1305 b.p.) | | | |
| 1832 b.p.; | | | | | |
| SEQ ID NO: 71 | 406 a.a.; | | | | |
| SEQ ID NO: 72 | 207222 | | | | |
| m TANGO 257 | FIG. 94 | (31-1248 b.p.) | | | |
| 1721 b.p.; | | | | | |
| SEQ ID NO: 73 | 370 a.a.; | | | | |
| SEQ ID NO: 74 | 207217 | | | | |
| h INTERCEPT 258 | FIG. 101 | (153-1262 b.p.) | | | |
| 1869 b.p.; | | | | | |
| SEQ ID NO: 75 | 370 a.a.; | | | | |
| SEQ ID NO: 76 | 207222 | | | | |
| m INTERCEPT 258 | FIG. 103 | (107-1288 b.p.) | | | |
| 1846 b.p.; | | | | | |
| SEQ ID NO: 77 | 394 a.a.; | | | | |
| SEQ ID NO: 78 | 207221 | | | | |
| h TANGO 204 | FIG. 111 | (99-890 b.p.) | | | |
| 3057 b.p.; | | | | | |
| SEQ ID NO: 79 | 264 a.a.; | | | | |
| SEQ ID NO: 80 | 207192 | | | | |
| m TANGO 204 | FIG. 115 | (81-872 b.p.) | | | |
| 1294 b.p.; | | | | | |
| SEQ ID NO: 81 | 264 a.a.; | | | | |
| SEQ ID NO: 82 | 207189 | | | | |
| h TANGO 206 | FIG. 118 | (99-1358 b.p.) | | | |
| 1840 b.p.; | | | | | |
| SEQ ID NO: 83 | 420 a.a.; | | | | |
| SEQ ID NO: 84 | 207223 | | | | |
| m TANGO 206 | FIG. 121 | (332-1591 b.p.) | | | |
| 2093 b.p.; | | | | | |
| SEQ ID NO: 85 | 420 a.a.; | | | | |
| SEQ ID NO: 86 | 207221 | | | | |
| h TANGO 209 | FIG. 124 | (194-1531 b.p.) | | | |
| 3117; | | | | | |
| SEQ ID NO: 87 | 446 a.a.; | | | | |
| SEQ ID NO: 88 | 207223 | | | | |
| m TANGO 209 | FIG. 128 | (187-1527 b.p.) | | | |
| 2810 b.p.; | | | | | |
| SEQ ID NO: 89 | 447 a.a.; | | | | |
| SEQ ID NO: 90 | 207221 | | | | |
| h TANGO 244 | FIG. 131 | (85-570 b.p.) | | | |
| 1513 b.p.; | | | | | |
| SEQ ID NO: 91 | 162 a.a.; | | | | |
| SEQ ID NO: 92 | 207223 | | | | |
| h TANGO 246 | FIG. 135 | (94-1080 b.p.) | | | |
| 1992 b.p.; | | | | | |
| SEQ ID NO: 93 | 329 a.a.; | | | | |
| SEQ ID NO: 94 | 207223 | | | | |
| h TANGO 275 | FIG. 139 | (23-3931 b.p.) | | | |
| 4225 b.p.; | | | | | |
| SEQ ID NO: 95 | 1289 a.a.; | | | | |
| SEQ ID NO: 96 | 207220 | | | | |
| m TANGO 275 | FIG. 146 | (157-3916 b.p.) | | | |
| 4376 b.p.; | | | | | |
| SEQ ID NO: 97 | 1253 a.a.; | | | | |
| SEQ ID NO: 98 | | | | | |
| h MANGO 245 | FIG. 147 | (105-1148 b.p.) | | | |
| 1356 b.p.; | | | | | |
| SEQ ID NO: 99 | 348 a.a.; | | | | |
| SEQ ID NO: 100 | 207223 | | | | |
| monkey | | | | | |
| MANGO 245 | FIG. 149 | (250-1236 b.p.) | | | |
| 1416 b.p.; | | | | | |
| SEQ ID NO: 101 | 329 a.a.; | | | | |
| SEQ ID NO: 102 | | | | | |
| m MANGO 245 | FIG. 153 | (29-625 b.p.) | | | |
| 625 b.p.; | | | | | |
| SEQ ID NO: 103 | 307 a.a.; | | | | |
| SEQ ID NO: 104 | | | | | |
| h INTERCEPT 340 | FIG. 157 | (1222-1944 b.p.) | | | |
| 3284 b.p.; | | | | | |
| SEQ ID NO: 105 | 241 a.a.; | | | | |
| SEQ ID NO: 106 | PTA-250 | | | | |
| h MANGO 003 | FIG. 160 | (57-1568 b.p.) | | | |
| 3169 b.p.; | | | | | |
| SEQ ID NO: 107 | 504 a.a.; | | | | |
| SEQ ID NO: 108 | 207178 | | | | |
| m MANGO 003 | FIG. 164 | (1-626 b.p.) | | | |
| 626 b.p.; | | | | | |
| SEQ ID NO: 109 | 208 a.a.; | | | | |
| SEQ ID NO: 110 | | | | | |
| h MANGO 347 | FIG. 166 | (31-444 b.p.) | | | |
| 1423 b.p.; | | | | | |
| SEQ ID NO: 111 | 138 a.a.; | | | | |
| SEQ ID NO: 112 | PTA-250 | | | | |
| h TANGO 272 | FIG. 169 | (230-3379 b.p.) | | | |
| 5036 b.p.; | | | | | |
| SEQ ID NO: 113 | 1050 a.a.; | | | | |
| SEQ ID NO: 114 | PTA 250 | | | | |
| m TANGO 272 | FIG. 172 | (1-1492 b.p.) | | | |
| 2569 b.p.; | | | | | |
| SEQ ID NO: 115 | 497 a.a.; | | | | |
| SEQ ID NO: 116 | | | | | |
| h TANGO 295 | FIG. 174 | (217-684 b.p.) | | | |
| 1497; | | | | | |
| SEQ ID NO: 117 | 156 a.a.; | | | | |
| SEQ ID NO: 118 | PTA-249 | | | | |
| h TANGO 354 | FIG. 177 | (62-976 b.p.) | | | |
| 1788 b.p.; | | | | | |
| SEQ ID NO: 119 | 305 a.a.; | | | | |
| SEQ ID NO: 120 | | | | | |
| h TANGO 378 | FIG. 180 | (42-1625 b.p.) | | | |
| 3258 b.p.; | | | | | |
| SEQ ID NO: 121 | 528 a.a.; | | | | |
| SEQ ID NO: 122 | | | | | |
| rat TANGO 272 | FIG. 189 | (925-2832 b.p.) | | | |
| 3567 b.p.; | | | | | |
| SEQ ID NO: 123 | 636 a.a.; | | | | |
| SEQ ID NO: 124 | | | | | |
| h TANGO 339 | FIG. 198 | (210-1019 b.p.) | | | |
| 2715 b.p.; | | | | | |
| SEQ ID NO: 125 | 270 a.a.; | | | | |
| SEQ ID NO: 126 | PTA-292 | | | | |
| h TANGO 358 | FIG. 202 | (184-429 b.p.) | | | |
| 1608 b.p.; | | | | | |
| SEQ ID NO: 127 | 82 a.a.; | | | | |
| SEQ ID NO: 128 | PTA-292 | | | | |
| h TANGO 365 | FIG. 204 | (56-550 b.p.) | | | |
| 1338 b.p.; | | | | | |
| SEQ ID NO: 129 | 165 a.a.; | | | | |
| SEQ ID NO: 130 | PTA-291 | | | | |
| h TANGO 368 | FIG. 206 | (152-328 b.p.) | | | |
| 983 b.p.; | | | | | |
| SEQ ID NO: 131 | 59 a.a.; | | | | |
| SEQ ID NO: 132 | PTA-291 | | | | |
| h TANGO 369 | FIG. 209 | (162-335 b.p.) | | | |
| 1119 b.p.; | | | | | |
| SEQ ID NO: 133 | 58 a.a.; | | | | |
| SEQ ID NO: 134 | PTA-295 | | | | |
| h TANGO 383 | FIG. 211 | (104-523 b.p.) | | | |
| 1386 b.p.; | | | | | |
| SEQ ID NO: 135 | 140 a.a.; | | | | |
| SEQ ID NO: 136 | PTA-295 | | | | |
| h MANGO 346 | FIG. 214 | (319-498 b.p.) | | | |
| 1196 b.p.; | | | | | |
| SEQ ID NO: 137 | 60 a.a.; | | | | |
| SEQ ID NO: 138 | PTA-291 | | | | |
| h MANGO 349 | FIG. 216 | (221-721 b.p.) | | | |
| 3649 b.p.; | | | | | |
| SEQ ID NO: 139 | 167 a.a.; | | | | |
| SEQ ID NO: 140 | PTA-295 | | | | |
| h INTERCEPT 307 | FIG. 218 | (45-1130 b.p.) | | | |
| 5058 b.p.; | | | | | |
| SEQ ID NO: 141 | 362 a.a.; | | | | |
| SEQ ID NO: 142 | PTA-455 | | | | |
| h MANGO 511 | FIG. 224 | (108-1004 b.p.) | | | |
| 1477 b.p.; | | | | | |
| SEQ ID NO: 143 | 299 a.a.; | | | | |
| SEQ ID NO: 144 | PTA-425 | | | | |
| TANGO 361 | FIG. 228 | (41-1309 b.p.) | | | |
| 5058 b.p.; | | | | | |
| SEQ ID NO: 145 | 423 a.a.; | | | | |
| SEQ ID NO: 146 | PTA-438 | | | | |
| TANGO 499, | | | | | |
| form 1, var. 1 | FIG. 230 | (83-844 b.p.) | | | |
| 1106 b.p.; | | | | | |
| SEQ ID NO: 147 | 254 a.a.; | | | | |
| SEQ ID NO: 148 | PTA-455 | | | | |
| TANGO 499, | | | | | |
| form 2, var. 3 | FIG. 234 | (144-830 b.p.) | | | |
| 1085 b.p.; | | | | | |
| SEQ ID NO: 149 | 229 a.a.; | | | | |
| SEQ ID NO: 150 | PTA-454 | | | | |
| h TANGO 315, form 1 | FIG. 237 | (1-888 b.p.) | | | |
| 1463 b.p.; | | | | | |
| SEQ ID NO: 151 | 296 a.a.; | | | | |
| SEQ ID NO: 152 | PTA-816 | | | | |
| h TANGO 315, form 2 | FIG. 243 | (58-888 b.p.) | | | |
| 1463 b.p.; | | | | | |
| SEQ ID NO: 153 | 277 a.a.; | | | | |
| SEQ ID NO: 154 | | | | | |
| h TANGO 330, form 1 | FIG. 249 | (2-2803 b.p.) | | | |
| 3042 b.p.; | | | | | |
| SEQ ID NO: 155 | 934 a.a.; | | | | |
| SEQ ID NO: 156 | PTA-816 | | | | |
| h TANGO 330, form 2 | FIG. 250 | (9-1448 b.p.) | | | |
| 3808; | | | | | |
| SEQ ID NO: 157 | 480 a.a.; | | | | |
| SEQ ID NO: 158 | | | | | |
| h TANGO 437, form 1 | FIG. 255 | (43-1815 b.p.) | | | |
| 4336 b.p.; | | | | | |
| SEQ ID NO: 159 | 591 a.a.; | | | | |
| SEQ ID NO: 160 | PTA-816 | | | | |
| TANGO 480 | FIG. 258 | (43-621 b.p.) | | | |
| 1912 b.p.; | | | | | |
| SEQ ID NO: 161 | 193 a.a.; | | | | |
| SEQ ID NO: 162 | PTA-816 | | | | |
| h TANGO 437, form 2 | FIG. 260 | (43-2298 b.p.) | | | |
| 3720 b.p.; | | | | | |
| SEQ ID NO: 163 | 752 a.a.; | | | | |
| SEQ ID NO: 164 | | | | | |

Various aspects of the invention are described in further detail in the following subsections:

### I. Isolated Nucleic Acid Molecules

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “isolated” when referring to a nucleic acid molecule does not include an isolated chromosome.

In instances wherein the nucleic acid molecule is a cDNA or RNA, e.g., mRNA, molecule, such molecules can include a poly A “tail”, or, alternatively, can lack such a 3′ tail. Although cDNA or RNA nucleotide sequences may be depicted herein with such tail sequences, it is to be understood that cDNA nucleic acid molecules of the invention are also intended to include such sequences lacking the depicted poly A tails.

All or a portion of the nucleic acid sequences of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a complement thereof, can be used as molecular weight markers when compared to a comparably sized nucleic acid sequence. Likewise, all or a portion of the amino acid sequences encoded by SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or a complement thereof can be used as molecular weight markers, in particular as molecular weight markers on SDS-PAGE electrophoresis.

A nucleic acid molecule of the present invention, e.g., a nucleic acid molecule having the nucleotide sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a complement thereof, can be isolated using standard molecular biology techniques and the SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, as a hybridization probe, nucleic acid molecules of the invention can be isolated using standard hybridization and cloning techniques (e.g. as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a portion thereof. A nucleic acid molecule which is complementary to a given nucleotide sequence is one which is sufficiently complementary to the given nucleotide sequence that it can hybridize to the nucleotide sequence under the conditions set forth herein, thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. In one embodiment, the oligonucleotide typically comprises a region of nucleotide sequence that hybridizes under stringent conditions to at least about 12, preferably about 25, more preferably about 50, 75, 100, 125, 150, 175, 200, 250, 300, 350 or 400 contiguous nucleotides of the sense or anti-sense sequence of INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499, of a naturally occurring mutant of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163. In another embodiment, the oligonucleotide comprises a region of nucleotide sequence that hybridizes under stringent conditions to at least 400, preferably 450, 500, 530, 550, 600, 700, 800, 900, 1000 or 1150 consecutive oligonucleotides of the sense or antisense sequence of a nucleic acid molecule of the invention or a naturally occurring mutant thereof.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, expressing the portion containing a reading frame of a polypeptide fragment (e.g., by recombinant expression in vitro) and assessing the activity of the encoded portion of the polypeptide fragment.

The invention further encompasses nucleic acid molecules that differ from the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, due to degeneracy of the genetic code and thus encode the same protein as that encoded by the nucleotide sequence any of the above.

In addition to the nucleotide sequences of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, it will be appreciated by those skilled in the art that DNA sequence polymorphisms that lead to changes in the amino acid sequence may exist within a population (e.g., the human population). Such genetic polymorphisms may exist among individuals within a population due to natural allelic variation.

An allele is one of a group of genes which occur alternatively at a given genetic locus. As used herein, the phrase “allelic variant” refers to a nucleotide sequence which occurs at a given locus or to a polypeptide encoded by the nucleotide sequence.

For example, TANGO 128 has been mapped to chromosome 4, between flanking markers WI-3936 and AFMCO27ZB9, and therefore, TANGO 128 family members can include nucleotide sequence polymorphisms (e.g., nucleotide sequences that vary from SEQ ID NO:5) that map to this chromosome 4 region (i.e., between markers WI-3936 and AFMCO17ZB9).

For example, TANGO 213 has been mapped to chromosome 17, in the region p13.3, between flanking markers WI-5436 and WI-6584, and therefore, TANGO 213 family members can include nucleotide sequence polymorphisms (e.g., nucleotide sequences that vary from SEQ ID NO:15) that map to this chromosome 17 region (i.e., between markers WI-5436 and WI-6584).

For example, a human TANGO 201 allele is one that maps to human chromosome 2 between markers D2S123 and D2S378. Likewise, a human TANGO 223 allele is one that maps to human chromosome 15q26 between flanking markers WI-3162 and WI-4919.

For example, human TANGO 216 has been mapped on radiation hybrid panels to the long arm of chromosome 4, in the region q11-13, between flanking markers GCT14E02 and jktbp-rs2, and therefore, human TANGO 216 family members can include nucleotide sequence polymorphisms (e.g., nucleotide sequences that vary from SEQ ID NO:51) that map to this chromosome 4 region (i.e., between markers GCT14E02 and jktbp-rs2).

For example, the human gene for TANGO 261 has been mapped on radiation hybrid panels to the long arm of chromosome 20, in the region q13.2-13.3, between flanking markers WI-3773 and AFMA202YB9, and therefore, human TANGO 261 family members can include nucleotide sequence polymorphisms (e.g., nucleotide sequences that vary from SEQ ID NO:55) that map to this chromosome 20 region (i.e., between markers WI-3773 and AFMA202YB9).

For example, the human gene for TANGO 262 has been mapped on radiation hybrid panels to the long arm of chromosome 14, in the region q23-q24, between flanking markers WI-6253 and WI-5815, and therefore, human TANGO 262 family members can include nucleotide sequence polymorphisms (e.g., nucleotide sequences that vary from SEQ ID NO:59) that map to this chromosome 14 region (i.e., between markers WI-6253 and WI-5815).

For example, the human gene for TANGO 267 was mapped on radiation hybrid panels to the long arm of chromosome X, in the region q12, between flanking markers WI-5587 and WI-5717, and therefore, human TANGO 267 family members can include nucleotide sequence polymorphisms (e.g., nucleotide sequences that vary from SEQ ID NO:65) that map to this chromosome X region (i.e., between markers WI-5587 and WI-5717).

For example, human TANGO 204 has been mapped on radiation hybrid panels to the long arm of chromosome 8q, in the region, between flanking markers D1Mit430 and D1Mit119, and therefore, human TANGO 204 family members can include nucleotide sequence polymorphisms (e.g., nucleotide sequences that vary from SEQ ID NO:79) that map to this chromosome 8 region (i.e., between markers D1Mit430 and D1Mit119).

For example, the human gene for TANGO 209 has been mapped on radiation hybrid panels to the long arm of chromosome 6, in the region q26-27, between flanking markers ATA22G07 and WI-9405, and therefore, human TANGO 209 family members can include nucleotide sequence polymorphisms (e.g., nucleotide sequences that vary from SEQ ID NO:87) that map to this chromosome 6 region (i.e., between markers ATA22G07 and WI-9405).

For example, TANGO 339 has been mapped to chromosome 10, and therefore TANGO 339, family members can include nucleotide sequence polymorphisms (e.g., nucleotide sequences that vary from SEQ ID NO: 125) that map to this chromosome 10 region, and such sequences represent allelic variants.

For example, INTERCEPT 307 has been mapped to chromosome 11, and therefore INTERCEPT 307 family members can include nucleotide sequence polymorphisms (e.g., nucleotide sequences that vary from SEQ ID NO: 141) that map to this chromosome 11 region (i.e., between markers D11S1357 and D11S1765), and such sequences represent INTERCEPT 307 allelic variants.

As used herein, the terms “gene” and “recombinant gene” refer to nucleic acid molecules comprising an open reading frame encoding a polypeptide of the invention.

Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention. In one embodiment, polymorphisms that are associated with a particular disease and/or disorder are used as markers to diagnose said disease or disorder. In a preferred embodiment, polymorphisms are used as a marker to diagnose abnormal coronary function such as atherosclerosis.

Moreover, nucleic acid molecules encoding proteins of the invention from other species (homologs), which have a nucleotide sequence which differs from that of the human or mouse protein described herein are intended to be within the scope of the invention. Nucleic acid molecules corresponding to natural allelic variants and homologs of a cDNA of the invention can be isolated based on their identity to the human nucleic acid molecule disclosed herein using the human cDNA, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 500, 600, 700, 800, 900, 1000, 1100, 1200, 1300, 1400, 1500, 1600, 1700, 1800, 1900 or 2000 contiguous nucleotides in length and hybridizes under stringent conditions to the nucleic acid molecule comprising the nucleotide sequence, preferably the coding sequence, of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a complement thereof.

Accordingly, in another embodiment, an isolated nucleic acid molecule of the invention is at least 25, 50, 100, 200, 300, 400, 500, 600, 700, 800 or 900 contiguous nucleotides in length and hybridizes under stringent conditions to the nucleic acid molecule comprising the nucleotide sequence, preferably the coding sequence, of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a complement thereof.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C. followed by one or more washes in 0.2×SSC, 0.1% SDS at 50-65° C. Preferably, an isolated nucleic acid molecule of the invention that hybridizes under stringent conditions to the sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., mouse and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499, yet retain biological activity. In one embodiment, the isolated nucleic acid molecule includes a nucleotide sequence encoding a protein that includes an amino acid sequence that is at least about 30%, 35%, 40%, 45%, 50%, 55%, 60%, 65%, 75%, 85%, 95%, or 98% identical to the amino acid sequence of a polypeptide of the invention.

An isolated nucleic acid molecule encoding a variant protein can be created by introducing one or more nucleotide substitutions, additions or deletions into the nucleotide sequence of INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499, such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine; methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein-protein interactions with proteins in a signaling pathway of the polypeptide of the invention such as in cells with the proteins encoded by the genes of the present invention; (2) the ability to bind a ligand of the polypeptide of the invention (i.e., in transmembrane proteins of the invention or alternatively, secreted proteins which are the ligand for a cellular receptor); or (3) the ability to bind to an intracellular target protein of the polypeptide of the invention. In yet another preferred embodiment, the mutant polypeptide can be assayed for the ability to modulate cellular proliferation, cellular migration, motility or chemotaxis, or cellular differentiation.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or ppl III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, (1988), Nature 334:585-591)) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific; ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Nat. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

In still other embodiments, the nucleotides of the invention including variants and derivatives can be used as vaccines, for example by genetic immunization. Genetic immunization is particularly advantageous as it stimulates a cytotoxic T-cell response but does not utilize live attenuated vaccines, which can revert to a virulent form and infect the host causing the very infection sought to be prevented. As used herein, genetic immunization comprises inserting the nucleotides of the invention into a host, such that the nucleotides are taken up by cells of the host and the proteins encoded by the nucleotides are translated. These translated proteins are then either secreted or processed by the host cell for presentation to immune cells and an immune reaction is stimulated. Preferably, the immune reaction is a cytotoxic T cell response, however, a humoral response or macrophage stimulation is also useful in preventing future infections. The skilled artisan will appreciate that there are various methods for introducing foreign nucleotides into a host animal and subsequently into cells for genetic immunization, for example, by intramuscular injection of about 50 μg of plasmid DNA encoding the proteins of the invention solubilized in 50 μl of sterile saline solution, with a suitable adjuvant (Weiner and Kennedy (1999) Scientific American 7:50-57; Lowrie et al., (1999) Nature 400:269-271).

### II. Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164. Other useful proteins are substantially identical (e.g., at least about 45%, preferably 55%, 65%, 75%, 85%, 95%, or 99%) to any of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

In another embodiment, the protein of the invention can be expressed as a dimer of itself. In this embodiment, a first domain of the protein is fused in frame to the same domain by a linker region. The linker can be a short flexible segment of amino acids, for example GGPGG or GPPGG, or a longer segment as needed. Alternatively, the first domain of the protein can be fused to a second domain of the protein, which is different than the first domain.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused with sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands. The immunoglobulin fusion protein can, for example, comprise a portion of a polypeptide of the invention fused with the amino-terminus or the carboxyl-terminus of an immunoglobulin constant region, as disclosed in U.S. Pat. No. 5,714,147, U.S. Pat. No. 5,116,964, U.S. Pat. No. 5,514,582, and U.S. Pat. No. 5,455,165.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g. Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

The polypeptides of the invention can exhibit post-translational modifications, including, but not limited to glycosylations, (e.g., N-linked or O-linked glycosylations), myristylations, palmitylations, acetylations and phosphorylations (e.g. serine/threonine or tyrosine). In one embodiment, the INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 polypeptides of the invention exhibit reduced levels of O-linked glycosylation and/or N-linked glycosylation relative to endogenously expressed polypeptides of the invention do not exhibit O-linked glycosylation or N-linked glycosylation.

The polypeptides of the invention can, for example, include modifications that can increase such attributes as stability, half-life, ability to enter cells and aid in administration, e.g., in vivo administration of the polypeptides of the invention. For example, polypeptides of the invention can comprise a protein transduction domain of the HIV TAT protein as described in Schwarze, et al. (1999 Science 285:1569-1572), thereby facilitating delivery of polypeptides of the invention into cells.

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, and encompasses an epitope of the protein such that an antibody raised against the peptide forms a specific immune complex with the protein.

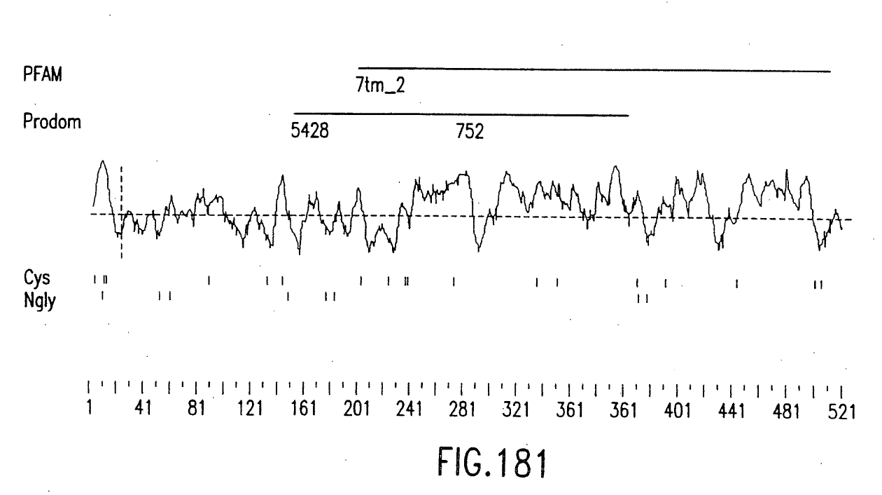

Preferred epitopes encompassed by the antigenic peptide are regions that are located on the surface of the protein, e.g., hydrophilic regions. These regions can be identified using hydropathy plots as described, for example, in the description of FIGS. 2, 4, 18, 19, 20, 21, 22, 23, 24, 40A, 46, 48, 50, 53, 55, 60, 65, 69, 73, 77, 85, 87, 93, 95, 102, 104, 112, 119, 125, 132, 136, 140, 148, 158, 161, 165, 167, 170, 173, 175, 178, 181, 196, 199, 203, 205, 207, 210, 212, 215, 217, 219, 225, 229, 231, 235, 238, 244, 256 or 259, or by similar analyses can be used to identify hydrophilic regions. In certain embodiments, the nucleic acid molecules of the invention are present as part of nucleic acid molecules comprising nucleic acid sequences that contain or encode heterologous (e.g., vector, expression vector, or fusion protein) sequences. These nucleotides can then be used to express proteins which can be used as immunogens to generate an immune response, or more particularly, to generate polyclonal or monoclonal antibodies specific to the expressed protein.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g. the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g. Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In addition, the gene sequences and gene products of the invention, including peptide fragments and fusion proteins thereof, and antibodies directed against said gene products and peptide fragments thereof, have applications for purposes independent of the role of the gene products, as described above. For example, INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 gene products, including peptide fragments, as well as specific antibodies thereto, can be used for construction of fusion proteins to facilitate recovery, detection, or localization of another protein of interest. In addition, genes and gene products of the invention can be used for genetic mapping. Finally, nucleic acids and gene products of the invention have generic uses, such as supplemental sources of nucleic acids, proteins and amino acids for food additives or cosmetic products.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-4 (“IL-4”) interleukin-6 (“IL-6”), interleukin-7 (“IL-7”) granulocyte macrophase colony stimulating factor (“GM-CSF”), granulocyte colony stimulating factor (“G-CSF”), interleukin-10 (“IL-10”), interleukin-12 (“IL-12”), interleukin-15 (“IL-15”), interferon-γ (“IFN-γ”), interferon-α (“IFN-α”), or other immune factors or growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with chemotherapeutic agents.

Alternatively, an antibody of the invention can be conjugated to a second antibody to form an “antibody heteroconjugate” as described by Segal in U.S. Pat. No. 4,676,980 or alternatively, the antibodies can be conjugated to form an “antibody heteropolymer” as described in Taylor et al., in U.S. Pat. Nos. 5,470,570 and 5,487,890.

An antibody with or without a therapeutic moiety conjugated to it can be used as a therapeutic that is administered alone or in combination with cytotoxic factor(s) and/or cytokine(s).

In yet a further aspect, the invention provides substantially purified antibodies or fragments thereof, including human or non-human antibodies or fragments thereof, which antibodies or fragments specifically bind to a polypeptide of the invention comprising an amino acid sequence selected from the group consisting of: the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited on Oct. 1, 1999 with the ATCC® and having the deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; a fragment of at least 15 contiguous amino acid residues of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; an amino acid sequence which is at least 95% identical to the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or to the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, under conditions of hybridization of 6×SSC at 45° C. and washing in 0.2×SSC, 0.1% SDS at 65° C. In various embodiments, the substantially purified antibodies of the invention, or fragments thereof, can be human, non-human, chimeric and/or humanized antibodies.

In another aspect, the invention provides human or non-human antibodies or fragments thereof, which antibodies or fragments specifically bind to a polypeptide comprising an amino acid sequence selected from the group consisting of: the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; a fragment of at least 15 contiguous amino acid residues of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; an amino acid sequence which is at least 95% identical to the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or to the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, under conditions of hybridization of 6×SSC at 45° C. and washing in 0.2×SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide of the invention comprising an amino acid sequence selected from the group consisting of: the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; a fragment of at least 15 contiguous amino acid residues of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; an amino acid sequence which is at least 95% identical to the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or the amino acid sequence encoded by the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or the cDNA insert of the plasmid deposited with the ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, under conditions of hybridization of 6×SSC at 45° C. and washing in 0.2×SSC, 0.1% SDS at 65° C. The monoclonal antibodies can be human, humanized, chimeric and/or non-human antibodies.

The substantially purified antibodies or fragments thereof specifically bind to a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the non-human antibodies or fragments thereof, and/or the monoclonal antibodies or fragments thereof, of the invention specifically bind to a secreted sequence, or alternatively, to an extracellular domain of the amino acid sequence of the invention.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention conjugated to a detectable substance, and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that specifically recognizes INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 polypeptides, derivatives thereof or fragments thereof, the method comprising immunizing a mammal with a polypeptide or polypeptide fragment. The polypeptide used as an immunogen comprises an amino acid sequence selected from the group consisting of: the amino acid sequence of any one of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or an amino acid sequence encoded by the cDNA of a clone deposited as ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; a fragment of at least 15 contiguous amino acid residues of the amino acid sequence of any one of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, an amino acid sequence which is at least 95% identical to the amino acid sequence of any one of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 2598, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or the cDNA of a clone deposited as ATCC® deposit number 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof, under conditions of hybridization of 6×SSC at 45° C. and washing in 0.2×SSC, 0.1% SDS at 65° C. After immunization, a sample is collected from the mammal that contains an antibody that specifically recognizes the immunogen. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

### III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g. E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMSI74(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kujan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell. Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g. the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous nucleic acid molecule encoding a polypeptide of the invention (e.g., INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499) within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499) and controls, modulates or activates the endogenous gene. For example, endogenous INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 which are normally “transcriptionally silent”, i.e., INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 genes may be activated by insertion of a promiscuous regulatory element that works across cell types.

A heterologous regulatory element may be inserted into a stable cell line or cloned microorganism, such that it is operatively linked with and activates expression of endogenous INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 genes, using techniques, such as targeted homologous recombination, which are well known to those of skill in the art, and described e.g., in Chappel, U.S. Pat. No. 5,272,071; PCT publication No. WO 91/06667, published May 16, 1991.

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequence encoding a polypeptide of the invention has been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. In addition to particular gene expression and/or polypeptide expression phenotypes, the transgenic animals of the invention can exhibit any of the phenotypes (e.g., processes, disorder symptoms and/or disorders), as are described in the sections above. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986) and Wakayama et al., (1999), Proc. Natl. Acad. Sci. USA, 96:14984-14989. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

### IV. Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELM (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

Antibodies or antibodies conjugated to therapeutic moieties can be administered to an individual alone or in combination with cytotoxic factor(s), chemotherapeutic drug(s), and/or cytokine(s). If the latter, preferably, the antibodies are administered first and the cytotoxic factor(s), chemotherapeutic drug(s) and/or cytokine(s) are administered thereafter within 24 hours. The antibodies and cytotoxic factor(s), chemotherapeutic drug(s) and/or cytokine(s) can be administered by multiple cycles depending upon the clinical response of the patient. Further, the antibodies and cytotoxic factor(s), chemotherapeutic drug(s) and/or cytokine(s) can be administered by the same or separate routes, for example, by intravenous, intranasal or intramuscular administration. Cytotoxic factors include, but are not limited to, TNF-α, TNF-β, IL-1, IFN-γ and IL-2. Chemotherapeutic drugs include, but are not limited to, 5-fluorouracil (5FU), vinblastine, actinomycin D, etoposide, cisplatin, methotrexate and doxorubicin. Cytokines include, but are not limited to, IL-2, IL-3, IL-4, IL-5, IL-6, IL-7, IL-8, IL-9, IL-10, IL-12 and IL-15.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### V. Uses and Methods of the Invention

The nucleic acid molecules, proteins, protein homologs, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used to (i) modulate cellular proliferation; (ii) modulate cellular differentiation; and/or (iii) modulate cellular adhesion. The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the and modulate activity of a protein of the invention.

This invention further pertains to novel agents identified by the above-described screening assays and uses thereof for treatments as described herein.

### A. Screening Assays

The invention provides a method (also referred to herein as a “screening assay”) for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of to the test compound to preferentially bind to the polypeptide or a biologically active portion thereof as compared to the known compound.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide protein to bind to or interact with a target molecule.

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic/enzymatic activity of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either the polypeptide of the invention or its target molecule to facilitate separation of complexed from uncomplexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-5-transferase fusion proteins or glutathione-5-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (statistically significantly greater) in the presence of the candidate compound than in its absence, the candidate compound is identified as a stimulator of the selected mRNA or protein expression. Alternatively, when expression of the selected mRNA or protein is less (statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

### B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

### 1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

For example, TANGO 128 has been mapped to chromosome 4, between flanking markers WI-3936 and AFMCO27ZB9; TANGO 213 has been mapped to chromosome 17, in the region p13.3, between flanking markers WI-5436 and WI-6584; human TANGO 201 maps to human chromosome 2 between markers D2S123 and D2S378; human TANGO 223 maps to human chromosome 15q26 between flanking markers WI-3162 and WI-4919; human TANGO 216 has been mapped to the long arm of chromosome 4, in the region q11-13, between flanking markers GCT14E02 and jktbp-rs2; human TANGO 261 has been mapped to the long arm of chromosome 20, in the region q13.2-13.3, between flanking markers WI-3773 and AFMA202YB9; human TANGO 262 has been mapped to the long arm of chromosome 14, in the region q23-q24, between flanking markers WI-6253 and WI-5815; human TANGO 267 was mapped to the long arm of chromosome X, in the region q12, between flanking markers WI-5587 and WI-5717; human TANGO 204 has been mapped to the long arm of chromosome 8q, in the region, between flanking markers D1Mit430 and D1Mit119; human TANGO 209 has been mapped to the long arm of chromosome 6, in the region q26-27, between flanking markers ATA22G07 and WI-9405; TANGO 339 has been mapped to chromosome 10; INTERCEPT 307 has been mapped to chromosome 11, between markers D11S1357 and D11S1765; human MANGO 511 was mapped (by BLASTing to MAPEST database) to human chromosome 11 between D11S1357 and D11S1765 (62.5-65 cM); and human TANGO 330, form 1 was mapped (by BLASTing to MAPEST database) to human chrmosome 11 between D11S1328 and D11S934 (128.4-131.7 cM).

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sublocalization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes (CITE), and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

A polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:3440. Alternatively, the presence of the polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

### 2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the noncoding regions. It is estimated that allelic variation between individual humans occurs with a frequency at about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, can comfortably provide positive individual identification with a panel of perhaps 10 to 1,000 primers which each yield a noncoding amplified sequence of 100 bases. If predicted coding sequences, such as those in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, are used, a more appropriate number of primers for positive individual identification would be 500 to 2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

### 3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individuals using this technique. Examples of polynucleotide reagents include the nucleic acid sequences of the invention or portions thereof, e.g. fragments derived from noncoding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

### C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention, such as a proliferative disorder, e.g., psoriasis or cancer, or an angiogenic disorder. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

Another aspect of the invention provides methods for expression of a nucleic acid or polypeptide of the invention or activity of a polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a polypeptide of the invention in clinical trials. These and other agents are described in further detail in the following sections.

### 1. Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO: 1, 3, 5, 7, 309, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163, or a portion thereof, such as an oligonucleotide of at least 15, 30, 50, 100, 250 or 500 contiguous nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a INTERCEPT 258, NTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 gene as discussed, for example, in sections above relating to uses of the sequences of the invention.

In another example, kits can be used to determine if a subject is suffering from or is at risk for disorders involving INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499.

In another example, kits can be used to determine if a subject is suffering from or is at risk for which are associated with aberrant INTERCEPT 258, INTERCEPT 307 and INTERCEPT 340, MANGO 003, MANGO 245, MANGO 346, MANGO 347, MANGO 349, and MANGO 511, and TANGO 128, TANGO 136, TANGO 140, TANGO 176, TANGO 197, TANGO 201, TANGO 204, TANGO 206, TANGO 209, TANGO 212, TANGO 213, TANGO 214, TANGO 216, TANGO 221, TANGO 222, TANGO 223, TANGO 224, TANGO 244, TANGO 246, TANGO 253, TANGO 257, TANGO 261, TANGO 262, TANGO 266, TANGO 267, TANGO 272, TANGO 275, TANGO 295, TANGO 315, TANGO 330, TANGO 339, TANGO 354, TANGO 358, TANGO 361, TANGO 365, TANGO 368, TANGO 369, TANGO 378, TANGO 383, TANGO 437, TANGO 480, and TANGO 499 family member activity and/or expression.

The kit, for example, can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

### 2. Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention, e.g., an immunologic disorder, or embryonic disorders. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

The prognostic assays described herein, for example, can be used to identify a subject having or at risk of developing disorders such as disorders discussed, for example, in sections above relating to uses of the sequences of the invention. For example, prognostic assays described herein can be used to identify a subject having or at risk of developing immunological disorders, e.g., autoimmune disorders (e.g., arthritis, graft rejection (e.g., allograft rejection), T cell disorders (e.g., ADS)), inflammatory disorders (e.g., bacterial infection, psoriasis, septicemia, cerebral malaria, inflammatory bowel disease, arthritis (e.g., rheumatoid arthritis, osteoarthritis)), and allergic inflammatory disorders (e.g., asthma, psoriasis), which are associated with aberrant TANGO 315, TANGO 330, TANGO 437, and TANGO 480 activity and/or expression.

In another example, prognostic assays described herein can be used to identify a subject having or at risk of developing brain-related disorders, inflammations (e.g., bacterial and viral meningitis, encephalitis, and cerebral toxoplasmosis), and tumors (e.g., astrocytoma), and to treat injury or trauma to the brain, which are associated with aberrant TANGO 330 family member activity and/or expression. In another example, prognostic assays described herein can be used to identify a subject having or at risk of developing adrenal-related disorders which are associated with aberrant TANGO 330 family member activity and/or expression. In another example, prognostic assays described herein can be used to identify a subject having or at risk of developing myeloid disorders such as acute or chronic myeloid leukemia which are associated with aberrant TANGO 315 family activity and/or expression. In another example, prognostic assays described herein can be used to identify a subject having or at risk of developing leptin-related disorders (e.g., neuroendocrine disorders, obesity, and anorexia nervosa) and embryonic disorders which are, associated with aberrant TANGO 315 family member activity and/or expression. In another example, prognostic assays described herein can be used to identify a subject having or at risk of developing ion transport disorders which are associated with aberrant TANGO 437 family member activity and/or expression. In yet another example, prognostic assays described herein can be used to identify a subject having or at risk of developing keratinocyte disorders such as squamous cell carcinoma which are associated with aberrant TANGO 480 family member activity and/or expression.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of “mismatch cleavage” entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature.

The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g. in clinical settings to diagnose patients exhibiting symptoms or family history of a disease or illness involving a gene encoding a polypeptide of the invention. Furthermore, any cell type or tissue, e.g., preferably peripheral blood leukocytes, in which the polypeptide of the invention is expressed may be utilized in the prognostic assays described herein.

### 3. Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

### 4. Monitoring of Effects During Clinical Trials

Monitoring the influence of agents (e.g., drugs, compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the preadministration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

### C. Methods of Treatment

The present invention provides for both prophylactic and therapeutic methods of treating a subject at risk of (or susceptible to) a disorder or having a disorder associated with aberrant expression or activity of a polypeptide of the invention, as discussed, for example, in sections above relating to uses of the sequences of the invention. For example, disorders characterized by aberrant expression or activity of the polypeptides of the invention include immunologic disorders, prostate disorders, endothelial cell disorders, developmental disorders, embryonic disorders, and neurological disorders. The nucleic acids, polypeptides, and modulators thereof of the invention can be used to treat immunologic diseases and disorders (e.g., monocyte disorders and platelet disorders), prostate disorders, embryonic disorders, and neurological disorders, as well as other disorders described herein.

### 1. Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The prophylactic agents described herein, for example, can be used to treat a subject at risk of developing disorders such as disorders discussed for example, in Sections above relative to the uses of the sequences of the invention. For example, an antagonist of an TANGO 315, TANGO 330, TANGO 437, and TANGO 480 protein may be used to modulate or treat an immunological disorder. The appropriate agent can be determined based on screening assays described herein.

### 2. Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., upregulates or downregulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or downregulated and/or in which increased activity is likely to have a beneficial effect. Conversely, inhibition of activity is desirable in situations in which activity or expression is abnormally high or upregulated and/or in which decreased activity is likely to have a beneficial effect.

This invention is further illustrated by the following examples which should not be construed as limiting. The contents of all references, patents and published patent applications cited throughout this application are hereby incorporated by reference.

### Deposit of Clones

Clones containing cDNA molecules encoding TANGO 128, TANGO 140-1, TANGO 140-2 and TANGO 197 were deposited with the American Type Culture Collection (Manassas, Va.) as composite deposits.

Clones encoding TANGO 128, TANGO 140-1, TANGO 140-2 and TANGO 197 were deposited on Nov. 20, 1998 with the American Type Culture Collection under Accession Number ATCC® 98999, (also referred to herein as mix EpDHMix1) from which each clone comprising a particular cDNA clone is obtainable. This deposit is a mixture of five strains, each carrying one recombinant plasmid harboring a particular cDNA clone. To distinguish the strains and isolate a strain harboring a particular cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions. The digest will liberate fragments as follows:

TANGO 128 (EpDH237) 2.8 kb and 4.3 kb

TANGO 140-1 (EpDH137) 1.6 kb and 3.0 kb

TANGO 140-2 (EpDH185) 3.4 kb and 4.3 kb

TANGO 197 (EpDH213) 2.3 kb and 3.0 kb

Clones containing cDNA molecules encoding human HtrA-2 (clone EpT214) was deposited with the American Type Culture Collection (Manassas, Va.) on Sep. 25, 1998 as Accession Number 98899, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 g/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| HtrA-2: | 2.6 kb |

Clones containing cDNA molecules encoding human TANGO 201 and TANGO 223 were deposited on Jan. 22, 1999 with the American Type Culture Collection (Manassas, Va.) under accession number ATCC™ 207081, from which each cDNA clone is obtainable. This deposit is a mixture of two strains, each carrying one recombinant plasmid. To distinguish the strains and isolate a strain harboring a particular cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions. The digest will liberate fragments as follows:

TANGO 201(EpT201), 2.2 kb

TANGO 223 (EpT223), 1.45 kb

Clones containing cDNA molecules encoding human TANGO 216, TANGO 261, TANGO 262, TANGO 266, and TANGO 267 (clones EpT216, EpT261, EpT262, EpT266, and EpT267, respectively), were deposited with the American Type Culture Collection (Manassas, Va.) on Mar. 26, 1999 as Accession Number 207176, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| TANGO 216 (EpT216): | 4.4 kb |
| TANGO 261 (EpT261): | 1.9 kb |
| TANGO 262 (EpT262): | 1.5 kb |
| TANGO 266 (EpT266): | .4 kb |
| TANGO 267 (EpT267): | 2.8 kb |

Clones containing cDNA molecules encoding human TANGO 253, (clone EpT253) human TANGO 257 (EpT257), and human INTERCEPT 258 (clone EpT258) were deposited with the American Type Culture Collection, 10801 University Boulevard, Manassas, Va., 20110-2209, on Apr. 21, 1999 as Accession Number 207222, as part of a composite deposit representing a mixture of strains, each carrying one recombinant plasmid harboring a particular cDNA clone.

For this composite deposit, to distinguish the strains and isolate a strain harboring a particular cDNA clone, an aliquot of the mixture can be streaked out to single colonies on nutrient medium (e.g., LB plates) supplemented with 100 g/ml ampicillin, single colonies grown, and then plasmid DNA extracted using a standard minipreparation procedure. Next, a sample of the DNA minipreparation can be digested with a combination of the restriction enzymes SalI, NotI, XbaI and EcorV and the resultant products resolved on a 0.8% agarose gel using standard DNA electrophoresis conditions. The digest liberates fragments as follows:

| | |
|---|---|
| Human TANGO 253 (clone EpT253): | 1.3 kb |
| Human TANGO 257 (clone EpT257): | 1.8 kb |
| Human INTERCEPT 258 (clone EpT258): | 1.0 kb and 0.85 kb |
| | (human INTERCEPT 258 |
| | has a EcorV cut site |
| | at about bp 1004). |

The identity of the strains can be inferred from the fragments liberated.

Clones containing cDNA molecules encoding mouse INTERCEPT 258 were deposited with the American Type Culture Collection (Manassas, Va.) on Apr. 21, 1999 as Accession Number 207221, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes SalI, and NotI, and the resultant products resolved on a 0.8% agarose gel using standard DNA electrophoresis conditions. The digest liberates fragments as follows:

| | |
|---|---|
| Mouse INTERCEPT 258 (clone EpT258): | 1.8 kb |

The identity of the strains can be inferred from the fragments liberated.

A clone containing a cDNA molecule encoding murine TANGO 253 (Clone Ep™ 253) was deposited with American Type Culture Collection, 10801 University Boulevard, Manassas, Va. 20110-2209, on Apr. 21, 1999 as Accession Number 207215.

A clone containing a cDNA molecule encoding murine TANGO 257 (Clone Ep™ 257) was deposited with American Type Culture Collection, 10801 University Boulevard, Manassas, Va. 20110-2209, on Apr. 21, 1999 as Accession Number 207217.

Clones containing cDNA molecules encoding human MANGO 003 were deposited with the American Type Culture Collection (ATCC® 10801 University Boulevard, Manassas, Va. 20110-2209) on Mar. 30, 1999 as Accession Number 207178, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 g/ml ampicillin, single colonies grown, and then plasmid DNA extracted using a standard minipreparation procedure. Next, a sample of the DNA minipreparation can be digested with a combination of the restriction enzymes Sal I and Not I, and the resultant products resolved on a 0.8% agarose gel using standard DNA electrophoresis conditions. The digest liberates fragments as follows:

| | |
|---|---|
| human MANGO 003 (clone EpthLa6a1): | 3.2 kB |

Clones containing cDNA molecules encoding human INTERCEPT 340, MANGO 347, and TANGO 272 were deposited with the American Type Culture Collection (ATCC® University Boulevard, Manassas, Va. 20110-2209) on Jun. 18, 1999 as Accession Number PTA-250, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 g/ml ampicillin, single colonies grown, and then plasmid DNA extracted using a standard minipreparation procedure. Next, a sample of the DNA minipreparation can be digested with a combination of the restriction enzymes Sal I and Not I, and the resultant products resolved on a 0.8% agarose gel using standard DNA electrophoresis conditions. The digest liberates fragments as follows:

| | |
|---|---|
| human INTERCEPT 340 (clone EpI340): | 3.3 kB |
| human MANGO 347 (clone EpM347): | 1.4 kB |
| human TANGO 272 (clone EpT272): | 5.0 kB |

The identity of the strains can be inferred from the fragments liberated.

Clones containing cDNA molecules encoding human TANGO 295, TANGO 354, and TANGO 378 were deposited with the American Type Culture Collection (ATCC® University Boulevard, Manassas, Va. 20110-2209) on Jun. 18, 1999 as Accession Number PTA-249, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 g/ml ampicillin, single colonies grown, and then plasmid DNA extracted using a standard minipreparation procedure. Next, a sample of the DNA minipreparation can be digested with a combination of the restriction enzymes Sal I and Not I, and the resultant products resolved on a 0.8% agarose gel using standard DNA electrophoresis conditions. The digest liberates fragments as follows:

| | |
|---|---|
| human TANGO 295 (clone EpT295): | 1.5 kB |
| human TANGO 354 (clone EpT354): | 1.8 kB |
| human TANGO 378 (clone EpT378): | 3.3 kB |

The identity of the strains can be inferred from the fragments liberated.

Clones containing cDNA molecules encoding TANGO 339 and TANGO 358 (clones EpT339 and EpT358, respectively), were deposited with the American Type Culture Collection (Manassas, Va.) on Jun. 29, 1999 as Accession Number PTA-292, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| TANGO 339 (EpT339): | 2.7 kb |
| TANGO 358 (EpT358): | 1.6 kb |

The identity of the strains can be inferred from the fragments liberated.

Clones containing cDNA molecules encoding MANGO 346, TANGO 365, and TANGO 368 (clones EpM346, EpT365, and EpT368, respectively), were deposited with the American Type Culture Collection (Manassas, Va.) on Jun. 29, 1999 as Accession Number PTA-291, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| MANGO 346 (EpM346): | 1.2 kb |
| TANGO 365 (EpT365): | 1.4 kb |
| TANGO 368 (EpT368): | 1.0 kb |

The identity of the strains can be inferred from the fragments liberated.

Clones containing cDNA molecules encoding MANGO 349, TANGO 369, and TANGO 383 (clones EpM349, EpT369, and EpT383, respectively), were deposited with the American Type Culture Collection (Manassas, Va.) on Jun. 29, 1999 as Accession Number PTA-295, as part of a composite deposit representing a mixture of four strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| MANGO 349 (EpM349): | 3.7 kb |
| TANGO 369 (EpT369): | 1.1 kb |
| TANGO 383 (EpT383): | 1.4 kb |

The identity of the strains can be inferred from the fragments liberated.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

Clones containing cDNA molecules encoding MANGO 511 (clone 511), were deposited with the American Type Culture Collection (Manassas, Va.) on Jul. 23, 1999 as Accession Number PTA-425, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates 1.5 kb fragments that correspond to MANGO 511 (511). The identity of the strain containing MANGO 511 can be inferred from the liberation of a fragment of the above identified size.

Clones containing cDNA molecules encoding INTERCEPT 307 and TANGO 361 (clones 307 and 361, respectively), were deposited with the American Type Culture Collection (Manassas, Va.) on Jul. 29, 1999 as Accession Number PTA-455, Accession Number PTA-438, and Accession Number PTA-438 respectively, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates fragments as follows:

| | |
|---|---|
| INTERCEPT 307 (307): | 2.0 kb |
| TANGO 361 (361): | 5.1 kb |

The identity of the strains can be inferred from the fragments liberated.

Clones containing cDNA molecules encoding TANGO 499 form 1, variant 1 (clone EpT499 form 1, variant 1), were deposited with the American Type Culture Collection (Manassas, Va.) on Aug. 5, 1999 as Accession Number PTA-455, as part of a composite deposit representing a mixture of three strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates 1.1 kb fragments that correspond to TANGO 499 form 1, variant 1 (EpT499 form 1, variant 1). The identity of the strain containing TANGO 499 form 1, variant 1 can be inferred from the liberation of a fragment of the above identified size.

Clones containing cDNA molecules encoding TANGO 499 form 2, variant 3 (clone EpT499 form 2, variant 3), were deposited with the American Type Culture Collection (Manassas, Va.) on Aug. 5, 1999 as Accession Number PTA-454, as part of a composite deposit representing a mixture of four strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes Sal I and Not I and the resultant products resolved on a 0.8% agarose gel using standard DNA electrophoresis conditions. The digest liberates 1.1 kb fragments that correspond to TANGO 499 form 2, variant 3 (EpT499 form 2, variant 3). The identity of the strain containing TANGO 499 form 2, variant 3 can be inferred from the liberation of a fragment of the above identified size.

Clones containing cDNA molecules encoding human TANGO 315, TANGO 330 form a, TANGO 330 form b, TANGO 437, and TANGO 480 (clones EpT315, 330a, 330b, 437, and 480, respectively) were deposited with the American Type Culture Collection (Manassas, Va.) on Oct. 1, 1999 as PTA-816, as part of a composite deposit representing a mixture of five strains, each carrying one recombinant plasmid harboring a particular cDNA clone.

To distinguish the strains and isolate a strain harboring a particular cDNA clone, an aliquot of the mixture can be streaked out to single colonies on nutrient medium (e.g., LB plates) supplemented with 100 μg/ml ampicillin, single colonies grown, and then plasmid DNA extracted using a standard minipreparation procedure. Next, a sample of the DNA minipreparation can be digested with a combination of the restriction enzymes SalI and NotI, and the resultant products resolved on a 0.8% agarose gel using standard DNA electrophoresis conditions. The digest liberates fragments as follows:

| | |
|---|---|
| 1. human TANGO 315 (clone EpT315): | 1.4 kb |
| 2. human TANGO 330 form 1 (clone 330a): | 3.0 kb |
| 3. human TANGO 330 form 2 (clone 330b): | 3.8 kb |
| 4. human TANGO 437 (clone 437): | 4.3 kb |
| 5. human TANGO 480 (clone 480): | 1.9 kb |

The identity of each of the strains can be inferred from the DNA fragments liberated.

All publications, patents and patent applications mentioned in this specification are herein incorporated by reference into the specification to the same extent as if each individual publication, patent or patent application was specifically and individually indicated to be incorporated herein by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 90% identical to the nucleotide sequence of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof;
b) a nucleic acid molecule comprising at least 15 nucleotide residues and having a nucleotide sequence identical to at least 15 consecutive nucleotide residues of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof;
c) a nucleic acid molecule which encodes a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816;
d) a nucleic acid molecule which encodes a fragment of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the fragment comprises at least 15 consecutive amino acid residues of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; and
e) a nucleic acid molecule which encodes a fragment of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the fragment comprises consecutive amino acid residues corresponding to at least half of the full length of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; and
f) a nucleic acid molecule which encodes a naturally occurring allelic variant of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, wherein the nucleic acid molecule hybridizes with a nucleic acid molecule consisting of the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof under stringent conditions.

2. The isolated nucleic acid molecule of claim 1, which is selected from the group consisting of:
a) a nucleic acid having the nucleotide sequence of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof; and
b) a nucleic acid molecule which encodes a polypeptide having the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816;
b) a naturally occurring allelic variant of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, wherein the polypeptide is encoded by a nucleic acid molecule which hybridizes with a nucleic acid molecule consisting of the nucleotide sequence of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof.

9. The isolated polypeptide of claim 8 having the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816;
b) a polypeptide comprising a fragment of the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, wherein the fragment comprises at least 10 contiguous amino acids of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164 and the amino acid sequence encoded by the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816; and
c) a naturally occurring allelic variant of a polypeptide comprising the amino acid sequence of any of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, and 164, or a complement thereof, wherein the polypeptide is encoded by a nucleic acid molecule which hybridizes with a nucleic acid molecule consisting of the nucleotide sequence of any of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 and the nucleotide sequence of any of the clones deposited as ATCC Accession numbers 98880, 98999, 202171, 98965, 98966, 98899, 207042, 207044, 207043, 207081, 207176, 207222, 207215, 207217, 207221, 207192, 207189, 207223, 207221, 207220, PTA-250, 207178, PTA-250, PTA-249, PTA-292, PTA-291, PTA-295, PTA-455, PTA-438, PTA-454, PTA-425, and PTA-816, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

18. The method of claim 17, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for an activity characteristic of the polypeptide.

19. A method for modulating the activity of a polypeptide of claim 8 comprising contacting a polypeptide or a cell expressing a polypeptide of claim 8 with a compound which binds with the polypeptide in a sufficient concentration to modulate the activity of the polypeptide.

20. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
